(19) United States
(12) Patent Application Publication
(10) Pub. No.: US 2004/0058338 A1
(43) Pub. Date: Mar. 25, 2004

(54) Novel proteins and nucleic acids encoding same

(76) Inventors: Michele L. Agee, Wallingford, CT (US); John P. Alsobrook II, Madison, CT (US); David W. Anderson, Branford, CT (US); Constance Berghs, New Haven, CT (US); Ferenc L. Boldog, North Haven, CT (US); Catherine E. Burgess, Wethersfield, CT (US); Elina Catterton, Madison, CT (US); Vincent A. DiPippo, East Haven, CT (US); Shlomit R. Edinger, New Haven, CT (US); Andrew Eisen, Rockville, MD (US); Karen Ellerman, Branford, CT (US); Esha A. Gangolli, Acton, MA (US); Valerie Gerlach, Branford, CT (US); Linda Gorman, Branford, CT (US); Bonnie Gould Rothberg, Guilford, CT (US); Xiaojia Sasha Guo, Branford, CT (US); John L. Herrmann, Guilford, CT (US); Yuan-Di Halvorsen, (US); Weizhen Ji, Branford, CT (US); Ramesh Kekuda, Norwalk, CT (US); Nikolai V. Khramtsov, Branford, CT (US); William J. LaRochelle, Madison, CT (US); Denise M. Lepley, Branford, CT (US); Li Li, Branford, CT (US); John R. MacDougall, Hamden, CT (US); Charles E. Miller, Guilford, CT (US); Tatiana Ort, Milford, CT (US); Muralidhara Padigaru, Branford, CT (US); Meera Patturajan, Branford, CT (US); Carol E. A. Pena, Guilford, CT (US); John A. Peyman, New Haven, CT (US); Daniel K. Rieger, Branford, CT (US); Mark E. Rothenberg, Clinton, CT (US); Suresh G. Shenoy, Branford, CT (US); Glennda Smithson, Guilford, CT (US); Steven K. Spaderna, Berlin, CT (US); Kimberly A. Spytek, New Haven, CT (US); David J. Stone, Guilford, CT (US); Raymond J. Taupier JR., East Haven, CT (US); Corine A.M. Vernet, Branford, CT (US); Edward Z. Voss, Wallingford, CT (US); Mei Zhong, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/307,817

(22) Filed: Dec. 2, 2002

Related U.S. Application Data
(60) Provisional application No. 60/336,881, filed on Dec. 3, 2001.
(60) Provisional application No. 60/336,820, filed on Dec. 5, 2001.
(60) Provisional application No. 60/361,770, filed on Mar. 5, 2002.
(60) Provisional application No. 60/364,238, filed on Mar. 13, 2002.
(60) Provisional application No. 60/338,285, filed on Dec. 7, 2001.
(60) Provisional application No. 60/383,829, filed on May 29, 2002.
(60) Provisional application No. 60/383,534, filed on May 28, 2002.
(60) Provisional application No. 60/338,318, filed on Dec. 7, 2001.
(60) Provisional application No. 60/404,676, filed on Aug. 20, 2002.
(60) Provisional application No. 60/353,288, filed on Feb. 1, 2002.
(60) Provisional application No. 60/362,230, filed on Mar. 5, 2002.
(60) Provisional application No. 60/364,181, filed on Mar. 13, 2002.
(60) Provisional application No. 60/339,022, filed on Dec. 10, 2001.
(60) Provisional application No. 60/353,286, filed on Feb. 1, 2002.
(60) Provisional application No. 60/364,978, filed on Mar. 15, 2002.
(60) Provisional application No. 60/338,989, filed on Dec. 10, 2001.
(60) Provisional application No. 60/359,956, filed on Feb. 27, 2002.
(60) Provisional application No. 60/360,964, filed on Feb. 28, 2002.
(60) Provisional application No. 60/405,698, filed on Aug. 23, 2002.
(60) Provisional application No. 60/339,314, filed on Dec. 11, 2001.
(60) Provisional application No. 60/339,517, filed on Dec. 11, 2001.
(60) Provisional application No. 60/361,256, filed on Feb. 28, 2002.
(60) Provisional application No. 60/339,611, filed on Dec. 11, 2001.
(60) Provisional application No. 60/359,914, filed on Feb. 27, 2002.
(60) Provisional application No. 60/405,400, filed on Aug. 23, 2002.
(60) Provisional application No. 60/339,516, filed on Dec. 11, 2001.
(60) Provisional application No. 60/359,626, filed on Feb. 26, 2002.
(60) Provisional application No. 60/361,264, filed on Feb. 28, 2002.
(60) Provisional application No. 60/365,025, filed on Mar. 15, 2002.
(60) Provisional application No. 60/405,684, filed on Aug. 23, 2002.
(60) Provisional application No. 60/340,981, filed on Dec. 12, 2001.
(60) Provisional application No. 60/340,565, filed on Dec. 14, 2001.
(60) Provisional application No. 60/359,671, filed on Feb. 26, 2002.
(60) Provisional application No. 60/360,924, filed on Feb. 28, 2002.
(60) Provisional application No. 60/381,004, filed on May 16, 2002.
(60) Provisional application No. 60/401,315, filed on Aug. 6, 2002.
(60) Provisional application No. 60/340,608, filed on Dec. 14, 2001.
(60) Provisional application No. 60/405,687, filed on Aug. 23, 2002.
(60) Provisional application No. 60/340,440, filed on Dec. 14, 2001.
(60) Provisional application No. 60/361,028, filed on Feb. 28, 2002.
(60) Provisional application No. 60/341,144, filed on Dec. 14, 2001.
(60) Provisional application No. 60/359,599, filed on Feb. 26, 2002.
(60) Provisional application No. 60/393,332, filed on Jul. 2, 2002.
(60) Provisional application No. 60/341,346, filed on Dec. 12, 2001.
(60) Provisional application No. 60/341,477, filed on Dec. 17, 2001.
(60) Provisional application No. 60/381,495, filed on May 17, 2002.
(60) Provisional application No. 60/401,788, filed on Aug. 7, 2002.
(60) Provisional application No. 60/341,540, filed on Dec. 17, 2001.
(60) Provisional application No. 60/383,744, filed on May 28, 2002.
(60) Provisional application No. 60/342,592, filed on Dec. 20, 2001.
(60) Provisional application No. 60/340,390, filed on Dec. 14, 2001.
(60) Provisional application No. 60/344,903, filed on Dec. 31, 2001.
(60) Provisional application No. 60/384,024, filed on May 29, 2002.
(60) Provisional application No. 60/373,288, filed on Apr. 17, 2002.
(60) Provisional application No. 60/380,981, filed on May 15, 2002.
(60) Provisional application No. 60/406,353, filed on Aug. 26, 2002.
(60) Provisional application No. 60/341,768, filed on Dec. 18, 2001.

(51) Int. Cl. C12Q 1/68; G01N 33/53; A61K 48/00; A61K 38/17; C07H 21/04; C12N 9/00; C07K 14/47

(52) U.S. Cl. 435/6; 435/69.1; 435/183; 435/320.1; 435/325; 514/12; 530/350; 536/23.2; 435/7.1

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application claims priority to provisional patent applications U.S. Ser. No. 60/336,881, filed Dec. 3, 2001; U.S. Ser. No. 60/336,820, filed Dec. 5, 2001; U.S. Ser. No. 60/361,770, filed Mar. 5, 2002; U.S. Ser. No. 60/364,238, filed Mar. 13, 2002; U.S. Ser. No. 60/338,285, filed Dec. 7, 2001; U.S. Ser. No. 60/383,829, filed May 29, 2002; U.S. Ser. No. 60/383,534, filed May 28, 2002; U.S. Ser. No. 60/338,318, filed Dec. 7, 2001; U.S. Ser. No. 60/404,676, filed Aug. 20, 2002; U.S. Ser. No. 60/353,288, filed February, 2001; U.S. Ser. No. 60/362,230, filed Mar. 5, 2002; U.S. Ser. No. 60/364,181, filed Mar. 13, 2002; U.S. Ser. No. 60/339,022, filed Dec. 10, 2001; U.S. Ser. No. 60/353,286, filed Feb. 1, 2002; U.S. Ser. No. 60/364,978, filed Mar. 15, 2002; U.S. Ser. No. 60/338,989, filed Dec. 10, 2001; U.S. Ser. No. 60/359,956, filed Feb. 27, 2002; U.S. Ser. No. 60/360,964, filed Feb. 28, 2002; U.S. Ser. No. 60/405,698, filed Aug. 23, 2002; U.S. Ser. No. 60/339,314, filed Dec. 11, 2001; U.S. Ser. No. 60/339,517, filed Dec. 11, 2001; U.S. Ser. No. 60/361,256, filed Feb. 28, 2002; U.S. Ser. No. 60/339,611, filed Dec. 11, 2001; U.S. Ser. No. 60/359,914, filed Feb. 27, 2002; U.S. Ser. No. 60/405,400, filed Aug. 23, 2002; U.S. Ser. No. 60/339,516, filed Dec. 11, 2001; U.S. Ser. No. 60/359,626, filed Feb. 26, 2002; U.S. Ser. No. 60/361,264, filed Feb. 28, 2002; U.S. Ser. No. 60/365,025, filed Mar. 15, 2002; U.S. Ser. No. 60/405,684, filed Aug. 23, 2002; U.S. Ser. No. 60/340,981, filed Dec. 12, 2001; U.S. Ser. No. 60/340,565, filed Dec. 14,2001; U.S. Ser. No. 60/359,671, filed Feb. 26, 2002; U.S. Ser. No. 60/360,924, filed Feb. 28, 2002; U.S. Ser. No. 60/381,004, filed May 16, 2002; U.S. Ser. No. 60/401,315, filed Aug. 6, 2002; U.S. Ser. No. 60/340,608, filed Dec. 14, 2001; U.S. Ser. No. 60/405,687, filed Aug. 23, 2002; U.S. Ser. No. 60/340,440, filed Dec. 14, 2001; U.S. Ser. No. 60/361,028, filed Feb. 28, 2002; U.S. Ser. No. 60/341,144, filed Dec. 14, 2001; U.S. Ser. No. 60/359,599, filed Feb. 26, 2002; U.S. Ser. No. 60/393,332, filed Jul. 2, 2002; U.S. Ser. No. 60/341,346, filed Dec. 12, 2001; U.S. Ser. No. 60/341,477, filed Dec. 17, 2001; U.S. Ser. No. 60/381,495, filed May 17, 2002; U.S. Ser. No. 60/401,788, filed Aug. 7, 2002; U.S. Ser. No. 60/341,540, filed Dec. 17, 2001; U.S. Ser. No. 60/383,744, filed May 28, 2002; U.S. Ser. No. 60/342,592, filed Dec. 20, 2001; U.S. Ser. No. 60/340,390, filed Dec. 14, 2001; U.S. Ser. No. 60/344,903, filed Dec. 31, 2001; U.S. Ser. No. 60/384,024, filed May 29, 2002; U.S. Ser. No. 60/373,288, filed Apr. 17, 2002; U.S. Ser. No. 60/380,981, filed May 15, 2002; U.S. Ser. No. 60/406,353, filed Aug. 26, 2002; U.S. Ser. No. 60/______ (given attorney docket number 21402-532 IFC-04), filed Oct. 31, 2002; and U.S. Ser. No. 60/341,768, filed Dec. 18, 2001; each of which is incorporated herein by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer between 1 and 188, or polypeptide sequences, which represents the group consisting of SEQ ID NO: 2n, wherein n is an integer between 1 and 188.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 188, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid | acid) | Homology |
| NOV1a | CG101719-02 | 1 | 2 | Fibroblast growth factor receptor 1 |
| | | | | IIIb-like protein |
| NOV1b | CG101719-04 | 3 | 4 | Fibroblast growth factor receptor 1 |
| | | | | IIIb-like protein |
| NOV1c | CG101719-05 | 5 | 6 | Fibroblast growth factor receptor 1 |
| | | | | IIIb-like protein |
| NOV1d | CG101719-01 | 7 | 8 | Fibroblast growth factor receptor 1 |
| | | | | IIIb-like protein |
| NOV1e | CG101719-03 | 9 | 10 | Fibroblast growth factor receptor 1 |
| | | | | IIIb-like protein |
| NOV2a | CG102006-01 | 11 | 12 | Human peroxiredoxin 2-like protein |
| NOV2b | CG102006-02 | 13 | 14 | Human peroxiredoxin 2-like protein |
| NOV2c | CG102006-03 | 15 | 16 | Human peroxiredoxin 2-like protein |
| NOV3a | CG127322-07 | 17 | 18 | Human kynurenine hydroxylase-like |
| | | | | protein |
| NOV3b | CG127322-01 | 19 | 20 | Human kynurenine hydroxylase-like |
| | | | | protein |
| NOV3c | CG127322-04 | 21 | 22 | Human kynurenine hydroxylase-like |
| | | | | protein |
| NOV3d | CG127322-03 | 23 | 24 | Human kynurenine hydroxylase-like |
| | | | | protein |
| NOV3e | 259357595 | 25 | 26 | Human kynurenine hydroxylase-like |
| | | | | protein |
| NOV3f | 255637561 | 27 | 28 | Human kynurenine hydroxylase-like |
| | | | | protein |
| NOV3g | 259357610 | 29 | 30 | Human kynurenine hydroxylase-like |
| | | | | protein |
| NOV3h | 259347911 | 31 | 32 | Human kynurenine hydroxylase-like |
| | | | | protein |
| NOV3i | 259347915 | 33 | 34 | Human kynurenine hydroxylase-like |
| | | | | protein |
| NOV3j | 260568545 | 35 | 36 | Human kynurenine hydroxylase-like |
| | | | | protein |
| NOV3k | 255872826 | 37 | 38 | Human kynurenine hydroxylase-like |
| | | | | protein |
| NOV31 | 255872853 | 39 | 40 | Human kynurenine hydroxylase-like |
| | | | | protein |
| NOV3m | CG127322-02 | 41 | 42 | Human kynurenine hydroxylase-like |
| | | | | protein |
| NOV3n | CG127322-05 | 43 | 44 | Human kynurenine hydroxylase-like |
| | | | | protein |
| NOV3o | CG127322-06 | 45 | 46 | Human kynurenine hydroxylase-like |
| | | | | protein |
| NOV4a | CG140122-07 | 47 | 48 | Human polyamine oxidase-like protein |
| NOV4b | CG140122-01 | 49 | 50 | Human polyamine oxidase-like protein |
| NOV4c | CG140122-03 | 51 | 52 | Human polyamine oxidase-like protein |
| NOV4d | CG140122-04 | 53 | 54 | Human Polyamine oxidase-like protein |
| NOV4e | 2468644043 | 55 | 56 | Human polyamine oxidase-like protein |
| NOV4f | 246864086 | 57 | 58 | Human polyamine oxidase-like protein |
| NOV4g | 258280083 | 59 | 60 | Human polyamine oxidase-like protein |
| NOV4h | 258329988 | 61 | 62 | Human potyamine oxidase-like protein |
| NOV4i | 258280066 | 63 | 64 | Human polyamine oxidase-like protein |
| NOV4j | 254047897 | 65 | 66 | Human polyamine oxidase-like protein |
| NOV4k | 258329988 | 67 | 68 | Human polyamine oxidase-like protein |
| NOV4l | 258280066 | 69 | 70 | Human polyamine oxidase-like protein |
| NOV4m | 258280083 | 71 | 72 | Human polyamine oxiclase-like protein |
| NOV4n | CG140122-02 | 73 | 74 | Human polyamine oxidase-like protein |
| NOV4o | CG140122-05 | 75 | 76 | Human polyamine oxidase-like protein |
| NOV4p | CG140122-06 | 77 | 78 | Human polyamine oxidase-like protein |
| NOV4q | CG140122-08 | 79 | 80 | Human polyamine oxidase-like protein |
| NOV5a | CG141O51-01 | 81 | 82 | Human glyceraldehyde-3-phosphate |
| | | | | dedrogenase-like protein |
| NOV6a | CG142427-05 | 83 | 84 | Human ATP-citrate (pro-S-)-lyase-like |
| | | | | protein |
| NOV6b | CG142427-02 | 85 | 86 | Human ATP-citrate (pro-S-)-lyase-like |
| | | | | protein |
| NOV6c | CG142427-03 | 87 | 88 | Human ATP-citrate (pro-S-)-lyase-like |
| | | | | protein |
| NOV6d | CG142427-04 | 89 | 90 | Human ATP-citrate (pro-S-)-lyase-like |
| | | | | protein |
| NOV6e | CG142427-01 | 91 | 92 | Human ATP-citrate (pro-S-)-lyase-like |
| | | | | protein |
| NOV7a | CG148010-03 | 93 | 94 | Human dacylglycerol acyltransferase |
| | | | | 2-like protein |
| NOV7b | CG148010-01 | 95 | 96 | Human dacylglycerol acyltransferase |
| | | | | 2-like protein |
| NOV7c | 246864114 | 97 | 98 | Human dacylglycerol acyltransferase |
| | | | | 2-like protein |
| NOV7d | 257448695 | 99 | 100 | Human dacylglycerol acyltransferase |
| | | | | 2-like protein |
| NOV7e | 259357675 | 101 | 102 | Human dacylglycerol acyltransferase |
| | | | | 2-like rotein |
| NOV7f | 254868590 | 103 | 104 | Human dacylglycerol acyltransferase |
| | | | | 2-like protein |
| NOV7g | CG148010-02 | 105 | 106 | Human dacylglycerol acyltransferase |
| | | | | 2-like protein |
| NOV7h | CG148010-04 | 107 | 108 | Human dacylglycerol acyltransferase |
| | | | | 2-like protein |
| NOV8a | CG148278-02 | 109 | 110 | Human longchain acyl CoA synthetase |
| | | | | 1-like protein |
| NOV8b | CG148278-01 | 111 | 112 | Human longchain acyl CoA synthetase |
| | | | | 1-like protein |
| NOV9a | CG152981-01 | 113 | 114 | Corticosteroid 11-beta-dehydrogenase, |
| | | | | isozyme 1-like protein |
| NOV9b | CG152981-02 | 115 | 116 | Corticosteroid 11-beta-dehydrogenase, |
| | | | | isozyme 1-like protein |
| NOV10a | CG159035-01 | 117 | 118 | Glucuronosyltransferase-like protein |
| NOV11a | CG159232-01 | 119 | 120 | Human cAMP-specific |
| | | | | phosphodiesterase 8 B1-like protein |
| NOV12a | CG159251-03 | 121 | 122 | O-Methyltansferase-like protein |
| NOV12b | CG159251-01 | 123 | 124 | O-Methyltansferase-like protein |
| NOVI2c | CG159251-02 | 125 | 126 | O-Methyitansferase-like protein |
| NOV13a | CG160563-01 | 127 | 128 | Monocarboxylate transporter 7-like |
| | | | | protein |
| NOV13b | CG160563-01 | 129 | 130 | Monocarboxylate transporter 7-like |
| | | | | protein |
| NOV14a | CG161527-01 | 131 | 132 | Sodium/potassium-transporting ATPase |
| | | | | alpha-4 chain-like protein |
| NOV15a | CG161579-01 | 133 | 134 | Dimethylaniline monooxygenase |
| | | | | (N-oxide-forming)-like protein |
| NOV16a | CG161650-0l | 135 | 136 | Cytochrome c oxidase polypeptide |
| | | | | VIc-like peptide |
| NOV17a | CG161733-01 | 137 | 138 | Axonemal dynein heavy chain-like |
| | | | | protein |
| NOV18a | CG161762-01 | 139 | 140 | Voltage-dependent anion-selective |
| | | | | channel protein 3-like protein |
| NOV19a | CG162855-01 | 141 | 142 | Neurolgin Y-like protein |
| NOV20a | CG163937-01 | 143 | 144 | Diamine N-acetyltransferase-like |
| | | | | protein |
| NOV21a | CG164449-02 | 145 | 146 | Granzyme H precursor-like protein |
| NOV21b | CG164449-01 | 147 | 148 | Granzyme H precursor-like protein |
| NOV22a | CG54007-06 | 149 | 150 | Carboxypeptidase X precursor-like |
| | | | | protein |
| NOV22b | CG54007-04 | 151 | 152 | Carboxypeptidase X precursor-like |
| | | | | protein |
| NOV22c | CG54007-01 | 153 | 154 | Carboxypeptidase X precursor-like |
| | | | | protein |
| NOV22d | CG54007-02 | 155 | 156 | Carboxypeptidase X precursor-like |
| | | | | protein |
| NOV22e | CG54007-03 | 157 | 158 | Carboxypeptidase X precursor-like |
| | | | | protein |
| NOV22f | CG54007-05 | 159 | 160 | Carboxypeptidase X precursor-like |
| | | | | protein |
| NOV22g | CG54007-07 | 161 | 162 | Carboxypeptidase X precursor-like |
| | | | | protein |
| NOV23a | CG55078-04 | 163 | 164 | Serine carboxypeptidase 1 |
| | | | | precursor-like protein |
| NOV23b | CG55078-01 | 165 | 166 | Serine carboxypeptidase 1 |
| NOV23c | CG55078-03 | 167 | 168 | precursor-like protein |
| NOV23d | 171094334 | 169 | 170 | Serine carboxypeptidase 1 |
| | | | | precursor-like protein |
| NOV23e | 171095197 | 171 | 172 | Serine carboxypeptidase 1 |
| | | | | precursor-like protein |
| NOV23f | 214374121 | 173 | 174 | Serine carboxypeptidase 1 |
| | | | | precursor-like protein |
| NOV23g | 171095146 | 175 | 176 | Serine carboxypeptidase 1 |
| | | | | precursor-like protein |
| NOV23h | 171095500 | 177 | 178 | Serine carboxypeptidase 1 |
| | | | | precursor-like protein |
| NOV23i | 171095508 | 179 | 180 | Serine carboxypeptidase 1 |
| | | | | precursor-like protein |
| NOV23j | 171095572 | 181 | 182 | Serine carboxypeptidase 1 |
| | | | | precursor-like protein |
| NOV23k | 171095162 | 183 | 184 | Serine carboxypeptidase 1 |
| | | | | precursor-like protein |
| N0V23l | 171095169 | 185 | 186 | Serine carboxypeptidase 1 |
| | | | | precursor-like protein |
| NOV23m | 222681273 | 187 | 188 | Serine carboxypeptidase 1 |
| | | | | precursor-like protein |
| NOV23n | 201536204 | 189 | 190 | Serine carboxypeptidase 1 |
| | | | | precursor-like protein |
| NOV23o | CG55078-02 | 191 | 192 | Serine carboxypeptidase 1 |
| | | | | precursor-like protein |
| NOV23p | CG55078-05 | 193 | 194 | Serine carboxypeptidase 1 |
| | | | | precursor-like protein |
| NOV23q | CG55078-06 | 195 | 196 | Serine carboxypeptidase 1 |
| | | | | precursor-like protein |
| NOV23r | CG55078-07 | 197 | 198 | Serine carboxypeptidase 1 |
| | | | | precursor-like protein |
| NOV24a | CG56149-07 | 199 | 200 | Nardilysin 1-like protein |
| NOV24b | CG56149-03 | 201 | 202 | Nardilysin 1-like protein |
| NOV24c | CG56149-01 | 203 | 204 | Nardilysin 1-like protein |
| NOV24d | CG56149-02 | 205 | 206 | Nardilysin 1-like protein |
| NOV24e | CG56149-04 | 207 | 208 | Nardilysin 1-like protein |
| NOV24f | CG56149-05 | 209 | 210 | Nardilysin 1-like protein |
| NOV24g | CG56149-06 | 211 | 212 | Nardilysin 1-like protein |
| NOV24h | CG56149-08 | 213 | 214 | Nardilysin 1-like protein |
| NOV25a | CG56216-01 | 215 | 216 | SERCA3-like protein |
| NOV25b | 222682222 | 217 | 218 | SERCA3-like protein |
| NOV25c | 248851003 | 219 | 220 | SERCA3-like protein |
| NOV25d | CG56216-02 | 221 | 222 | SERCA3-like protein |
| NOV26a | CG56230-01 | 223 | 224 | Olfactory receptor-like protein |
| NOV27a | CG56246-04 | 225 | 226 | Human carboxypeptidase A2-like |
| | | | | protein |
| NOV27b | CG56246-02 | 227 | 228 | Human carboxypeptidase A2-like |
| | | | | protein |
| NOV27c | 171092849 | 229 | 230 | Human carboxypeptidase A2-like |
| | | | | protein |
| NOV27d | 183852323 | 231 | 232 | Human carboxypeptidase A2-like |
| NOV27e | 173229182 | 233 | 234 | Human carboxypeptidase A2-like |
| NOV27f | 173172465 | 235 | 236 | Human carboxypeptidase A2-like |
| NOV27g | CG56246-01 | 237 | 238 | Human carboxypeptidase A2-like |
| NOV27h | 274057795 | 239 | 240 | Human carboxypeptidase A2-like |
| | | | | protein |
| NOV27i | 274057823 | 241 | 242 | Human carboxypeptidase A2-like |
| | | | | protein |
| NOV27j | 274057830 | 243 | 244 | Human carboxypeptidase A2-like |
| | | | | protein |
| NOV27k | 274057838 | 245 | 246 | Human carboxypeptidase A2-like |
| | | | | protein |
| N0V27l | CG56246-03 | 247 | 248 | Human carboxypeptidase A2-like |
| | | | | protein |
| NOV27m | CG56246-05 | 249 | 250 | Human carboxypeptidase A2-like |
| | | | | protein |
| NOV28a | CG57417-05 | 251 | 252 | Human SERCA 1-like protein |
| NOV28b | CG57417-03 | 253 | 254 | Human SERCA 1-like protein |
| NOV28c | 255169268 | 255 | 256 | Human SERCA 1-like protein |
| NOV28d | CG57417-01 | 257 | 258 | Human SERCA 1-like protein |
| NOV28e | 181356924 | 259 | 260 | Human SERCA 1-like protein |
| NOV28f | 255169268 | 261 | 262 | Human SERCA 1-like protein |
| NOV28g | 206977032 | 263 | 264 | Human SERCA 1-like protein |
| NOV28h | 201190923 | 265 | 266 | Human SERCA 1-like protein |
| NOV28i | CG57417-02 | 267 | 268 | Human SERCA 1-like protein |
| N0V28j | CG57417-04 | 269 | 270 | Human SERCA 1-like protein |
| NOV28k | CG57417-06 | 271 | 272 | Human SERCA 1-like protein |
| N0V28l | CG57417-07 | 273 | 274 | Human SERCA 1-like protein |
| NOV29a | CG93541-05 | 275 | 276 | Human autotaxin-t-like protein |
| NOV29b | CG93541-01 | 277 | 278 | Human autotaxin-t-like protein |
| NOV29c | CG93541-02 | 279 | 280 | Human autotaxin-t-like protein |
| NOV29d | CG93541-03 | 281 | 282 | Human autotaxin-t-like protein |
| NOV29e | CG93541-04 | 283 | 284 | Human autotaxin-t-like protein |
| NOV29f | CG93541-06 | 285 | 286 | Human autotaxin-t-like protein |
| NOV30a | CG93735-05 | 287 | 288 | Human adenylate kinase 3 alpha-like |
| | | | | protein |
| NOV30b | CG93735-01 | 289 | 290 | Human AK3 alpha-like protein |
| NOV30c | 171094650 | 291 | 292 | Human AK3 alpha-like protein |
| NOV30d | 173172155 | 293 | 294 | Human AK3 alpha-like protein |
| NOV30e | 195803542 | 295 | 296 | Human AK3 alpha-like protein |
| NOV30f | 171093359 | 297 | 298 | Human AK3 alpha-like protein |
| NOV30g | 171065502 | 299 | 300 | Human AK3 alpha-like protein |
| NOV30h | 171093533 | 301 | 302 | Human AK3 alpha-like protein |
| NOV30i | 171094630 | 303 | 304 | Human AK3 alpha-like protein |
| NOV30j | 278391231 | 305 | 306 | Human AK3 alpha-like protein |
| NOV30k | 283291704 | 307 | 308 | Human AK3 alpha-like protein |
| NOV30I | CG93735-02 | 309 | 310 | Human AK3 alpha-like protein |
| NOV30m | CG93735-03 | 311 | 312 | Human AK3 alpha-like protein |
| NOV30n | CG93735-04 | 313 | 314 | Human AK3 alpha-like protein |
| NOV30o | CG93735-06 | 315 | 316 | Human AK3 alpha-like protein |
| NOV31a | CG93817-01 | 317 | 318 | GPCR olfactory receptor-like protein |
| NOV32a | CG96859-03 | 319 | 320 | Human HMG CoA lyase precursor-like |
| | | | | protein |
| NOV32b | 233169960 | 321 | 322 | Human HMG CoA lyase precursor-like |
| | | | | protein |
| NOV32c | 223316987 | 323 | 324 | Human HMG CoA lyase precursor-like |
| | | | | protein |
| NOV32d | CG96859-01 | 325 | 326 | Human HMG CoA lyase precursor-like |
| | | | | protein |
| NOV32e | CG96859-02 | 327 | 328 | Human HMG CoA lyase precursor-like |
| | | | | protein |
| NOV32f | CG96859-04 | 329 | 330 | Human HMG CoA lyase precursor-like |
| | | | | protein |
| NOV32g | CG96859-05 | 331 | 332 | Human HMG CoA lyase precursor-like |
| | | | | protein |
| NOV32h | CG96859-06 | 333 | 334 | Human HMG CoA lyase precursor-like |
| | | | | protein |
| NOV32i | CG96859-07 | 335 | 336 | Human HMG CoA lyase precursor-like |
| | | | | protein |
| NOV32j | CG96859-08 | 337 | 338 | Human HMG CoA lyase precursor-like |
| | | | | protein |
| NOV32k | CG96859-09 | 339 | 340 | Human HMG CoA lyase precursor-like |
| | | | | protein |
| NOV33a | CG105355-03 | 341 | 342 | Human aryl hydrocarbon (Ah) receptor- |
| | | | | like protein |
| NOV33b | CG105355-01 | 343 | 344 | Human Ah receptor-like protein |
| NOV33c | CG105355-02 | 345 | 346 | Human Ah receptor-like protein |
| NOV33d | CG105355-04 | 347 | 348 | Human Ah receptor-like protein |
| NOV34a | CG96736-02 | 349 | 350 | Human neutral amino acid transporter |
| | | | | B(0)-like protein |
| NOV34b | CG96736-01 | 351 | 352 | Human neutral ATB(0)-like protein |
| NOV34c | 210203253 | 353 | 354 | Human neutral ATB(0)-like protein |
| NOV34d | 210203261 | 355 | 356 | Human neutral ATB(0)-like protein |
| NOV35a | CG97025-04 | 357 | 358 | Human hydroxymethylglutaryl-CoA |
| | | | | synthase-like protein |
| NOV35b | CG97025-01 | 359 | 360 | Human HMG-CoA synthase-like |
| | | | | protein |
| NOV35c | 254869578 | 361 | 362 | Human HMG-CoA synthase-like |
| | | | | protein |
| NOV35d | 253174237 | 363 | 364 | Human HMG-CoA synthase-like |
| | | | | protein |
| NOV35e | 256420363 | 365 | 366 | Human HMG-CoA synthase-like |
| | | | | protein |
| NOV35f | 255667064 | 367 | 368 | Human HMG-CoA synthase-like |
| | | | | protein |
| NOV35g | 228832739 | 369 | 370 | Human HMG-CoA synthase-like |
| | | | | protein |
| NOV35h | CG97025-02 | 371 | 372 | Human HMG-CoA synthase-like |
| | | | | protein |
| NOV35i | CG97025-03 | 373 | 374 | Human HMG-CoA synthase-like |
| | | | | protein |
| NOV35j | CG97025-05 | 375 | 376 | Human HMG-CoA synthase-like |
| | | | | protein |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease;

multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins.

Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers. SNP analysis for each NOVX, if applicable,, is presented in Example D.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 188; (b) a variant of a mature form of the amino acid sequence selected from the group consisting of SEQ ID NO: 2n, wherein n is an integer between 1 and 188, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 188; (d) a variant of the amino acid sequence selected from the group consisting of SEQ ID NO:2n, wherein n is an integer between 1 and 188 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 188; (b) a variant of a mature form of the amino acid sequence selected from the group consisting of SEQ ID NO: 2n, wherein n is an integer between 1 and 188 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 188; (d) a variant of the amino acid sequence selected from the group consisting of SEQ ID NO: 2n, wherein n is an integer between 1 and 188, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 188 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 188; (b) a nucleotide sequence wherein one or more nucleotides in the nucleotide sequence selected from the group consisting of SEQ ID NO: 2n−1, wherein n is an integer between 1 and 188 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 188; and (d) a nucleic acid fragment wherein one or more nucleotides in the nucleotide sequence selected from the group consisting of SEQ ID NO: 2n−1, wherein n is an integer between 1 and 188 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g, cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 188, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 188, as a hybridization probe, NOVX molecules can be isolated using standard hybridization and cloning techniques (e.g., as described in Sambrook, et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 188, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 188, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 188, is one that is sufficiently complementary to the nucleotide sequence of SEQ ID NO:2n−1, wherein n is an integer between 1 and 188, that it can hydrogen bond with few or no mismatches to the nucleotide sequence shown in SEQ ID NO:2n−1, wherein n is an integer between 1 and 188, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 188, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 188; or an anti-sense strand nucleotide sequence of SEQ ID NO:2n−1, wherein n is an integer between 1 and 188; or of a naturally occurring mutant of SEQ ID NO:2n−1, wherein n is an integer between 1 and 188.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 188, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 188, due to degeneracy of the genetic code and thus encode the same NOVX proteins as that encoded by the nucleotide sequences of SEQ ID NO:2n−1, wherein n is an integer between 1 and 188. In another embodiment, an isolated nucleic acid molecule of the invention has a nucleotide sequence encoding a protein having an amino acid sequence of SEQ ID NO:2n, wherein n is an integer between 1 and 188.

In addition to the human NOVX nucleotide sequences of SEQ ID NO:2n−1, wherein n is an integer between 1 and 188, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 188, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 188. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 188, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 188, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Krieger, 1990; Gene Transfer and Expression, A Laboratory Manual, Stockton Press, NY.

In a third embodiment, a nucleic acid that is hybridizable to the nucleic acid molecule comprising the nucleotide sequences of SEQ ID NO:2n−1, wherein n is an integer between 1 and 188, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCI (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 188, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 188. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are not particularly amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 188, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 188. Preferably, the protein encoded by the nucleic acid molecule is at least about 60% homologous to SEQ ID NO:2n, wherein n is an integer between 1 and 188; more preferably at least about 70% homologous to SEQ ID NO:2n, wherein n is an integer between 1 and 188; still more preferably at least about 80% homologous to SEQ ID NO:2n, wherein n is an integer between 1 and 188; even more preferably at least about 90% homologous to SEQ ID NO:2n, wherein n is an integer between 1 and 188; and most preferably at least about 95% homologous to SEQ ID NO:2n, wherein n is an integer between 1 and 188.

An isolated nucleic acid molecule encoding a NOVX protein homologous to the protein of SEQ ID NO:2n, wherein n is an integer between 1 and 188, can be created by introducing one or more nucleotide substitutions, additions or deletions into the nucleotide sequence of SEQ ID NO:2n−1, wherein n is an integer between 1 and 188, such that one or more amino acid substitutions, additions or deletions are introduced into the encoded protein.

Mutations can be introduced any one of SEQ ID NO:2n−1, wherein n is an integer between 1 and 188, by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 188, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the. nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 188, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 188, or antisense nucleic acids complementary to a NOVX nucleic acid sequence of SEQ ID NO:2n−1, wherein n is an integer between 1 and 188, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35,40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 188). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 188. The invention also includes a mutant or variant protein any of whose residues may be changed from the corresponding residues shown in any one of SEQ ID NO:2n, wherein n is an integer between 1 and 188, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 188) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 188. In other embodiments, the NOVX protein is substantially homologous to SEQ ID NO:2n, wherein n is an integer between 1 and 188, and retains the functional activity of the protein of SEQ ID NO:2n, wherein n is an integer between 1 and 188, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 188, and retains the functional activity of the NOVX proteins of SEQ ID NO:2n, wherein n is an integer between 1 and 188.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 188.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 188, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 188, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (ire., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and 186Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1 and 188, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 188), but more preferably, is a non-human homologue of a human NOVX gene. For example, a mouse homologue of human NOVX gene of SEQ ID NO:2n−1, wherein n is an integer between 1 and 188, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdernal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1941. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g. Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of-the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 188, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 188, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 188, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A: Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | SEQ ID NO:1 | 494 bp | |
|---|---|---|---|
| NOV1a, | ATGTGGAGCTGGAAGTGCCTCCCCTTCTGGGCTGTGCTGGTCACAGCCACACTCTGCACCGCTAGGCC | | |
| CG101719-02 | | | |
| DNA Sequence | GTCCCCGACCTTGCCTGAACAAGCCCAGCCCTGGGGAGCCCCTGTGGAAGTGGAGTCCTTCCTGGTCC | | |
| | ACCCCGGTGACCTGCTGCAGCTTCGCTGTCGGCTGCGGGACGATGTGCAGAGCATCAACTGGCTGCGG | | |
| | GACGGGGTGCAGCTGGCGGAAAGCAACCGCACCCGCATCACAGGGGAGGAGGTGGAGGTGCAGGACTC | | |
| | CGTGCCCGCAGACTCCGGCCTCTATGCTTGCGTAACCAGCAGCCCCTCGGGCAGTGACACCACCTACT | | |
| | TCTCCGTCAATGTTTCAGCTTGCCCAGATCTCCAGGAGGCTAAGTGGTGCTCGGCCAGCTTCCACTCC | | |
| | ATCACTCCCTTGCCATTTGGACTTGGTACTCGGCTTAGTGATTAGAGGCCCTGAACAGGTGGTGGTAT | | |
| | CCCTGCTCTGCTGGAGAG | | |
| | ORF Start: ATG at 1 | | ORF Stop: TAG at 451 |
| | SEQ ID NO:2 | 150 aa | MW at 16470.3 kD |
| NOV1a, | MWSWKCLPFWAVLVTATLCTARPSPTLPEQAQPWGAPVEVESFLVHPGDLLQLRCRLRDDVQSINWLR | | |
| CG101719-02 | | | |
| Protein | DGVQLAESNRTRITGEEVEVQDSVPADSGLYACVTSSPSGSDTTYFSVNVSACPDLQEAKWCSASFHS | | |
| Sequence | | | |
| | ITPLPFGLGTRLSD | | |
| | SEQ ID NO:3 | 2737 bp | |
| NOV1b, | CGAGGCGGAACCTCCAGCCCGAGCGAGGGTCAGTTTGAAAAGGAGGATCGAGCTCACTGTGGAGTAT | | |
| CG101719-04 | CCATGGAGATGTGGAGCCTTGTCACCAACCTCTAACTGCAGAACTGGGATGTGGAGCTGGAAGTGCC | | |
| DNA Sequence | TCCTCTTCTGGGCTGTGCTGGTCACAGCCACACTCTGCACCGCTAGGCCGTCCCCGACCTTGCCTGA | | |
| | ACAAGCCCAGCCCTGGGGAGCCCCTGTGGAAGTGGAGTCCTTCCTGGTCCACCCCGGTGACCTGCTG | | |
| | CAGCTTCGCTGTCGGCTGCGGGACGATGTGCAGAGCATCAACTGGCTGCGGGACGGGGTGCAGCTGG | | |
| | CGGAAAGCAACCGCACCCGCATCACAGGGGAGGAGGTGGAGGTGCAGGACTCCGTGCCCGCAGACTC | | |
| | CGGCCTCTATGCTTGCGTAACCAGCAGCCCCTCGGGCAGTGACACCACCTACTTCTCCGTCAATGTT | | |
| | TCAGATGCTCTCCCCTCCTCGGAGGATGATGATGATGATGATGACTCCTCTTCAGAGGAGAAAGAAA | | |
| | CAGATAACACCAAACCAAACCGTATGCCCGTAGCTCCATATTGGACATCCCCAGAAAAGATGGAAAA | | |
| | GAAATTGCATGCAGTGCCGGCTGCCAAGACAGTGAAGTTCAAATGCCCTTCCAGTGGGACCCCAAAC | | |
| | CCCACACTGCGCTGGTTGAAAAATGGCAAAGAATTCAAACCTGACCACAGAATTGGAGGCTACAAGG | | |
| | TCCGTTATGCCACCTGGAGCATCATAATGGACTCTGTGGTGCCCTCTGACAAGGGCAACTACACCTG | | |
| | CATTGTGGAGAATGAGTACGGCAGCATCAACCACACATACCAGCTGGATGTCGTGGAGCGGTCCCCT | | |
| | CACCGGCCCATCCTGCAAGCAGGGTTGCCCGCCAACAAAACAGTGGCCCTGGGTAGCAACGTGGAGT | | |
| | TCATGTGTAAGGTGTACAGTGACCCGCAGCCGCACATCCAGTGGCTAAAGCACATCGAGGTGAATGG | | |
| | GAGCAAGATTGGCCCAGACAACCTGCCTTATGTCCAGATCTTGAAGCATTCGGGGATTAATAGCTCG | | |
| | GATGCGGAGGTGCTGACCCTGTTCAATGTGACAGAGGCCCAGAGCGGGGAGTATGTGTGTAAGGTTT | | |
| | CCAATTATATTGGTGAAGCTAACCAGTCTGCGTGGCTCACTGTCACCAGACCTGTGGCAAAAGCCCT | | |
| | GGAAGAGAGGCCGGCAGTGATGACCTCGCCCCTGTACCTGGAGATCATCATCTATTGCACAGGGGCC | | |
| | TTCCTCATCTCCTGCATGGTGGGGTCGGTCATCGTCTACAAGATGAAGAGTGGTACCAAGAAGAGTG | | |
| | ACTTCCACAGCCAGATGGCTGTGCACAAGCTGGCCAAGAGCATCCCTCTGCGCAGACAGGTAACAGT | | |
| | GTCTGCTGACTCCAGTGCATCCATGAACTCTGGGGTTCTTCTGGTTCGGCCATCACGGCTCTCCTCC | | |
| | AGTGGGACTCCCATGCTAGCAGGGGTCTCTGAGTATGAGCTTCCCGAAGACCCTCGCTGGGAGCTGC | | |
| | CTCGGGACAGACTGGTCTTAGGCAAACCCCTGGGAGAGGGCTGCTTTGGGCAGGTGGTGTTGGCAGA | | |
| | GGCTATCGGGCTGGACAAGGACAAACCCAACCGTGTGACCAAAGTGGCTGTGAAGATGTTGAAGTCG | | |
| | GACGCAACAGAGAAAGACTTGTCAGACCTGATCTCAGAAATGGAGATGATGAAGATGATCGGGAAGC | | |
| | ATAAGAATATCATCAACCTGCTGGGGGCCTGCACGCAGGATGGTCCCTTGTATGTCATCGTGGAGTA | | |
| | TGCCTCCAAGGGCAACCTGCGGGAGTACCTGCAGGCCCGGAGGCCCCCAGGGCTGGAATACTGCTAC | | |
| | AACCCCAGCCACAACCCAGAGGAGCAGCTCTCCTCCAAGGACCTGGTGTCCTGCGCCTACCAGGTGG | | |
| | CCCGAGGCATGGAGTATCTGGCCTCCAAGAAGTGCATACACCGAGACCTGGCAGCCAGGAATGTCCT | | |
| | GGTGACAGAGGACAATGTGATGAAGATAGCAGACTTTGGCCTCGCACGGGACATTCACCACATCGAC | | |
| | TACTATAAAAAGACAACCAACGGCCGACTGCCTGTGAAGTGGATGGCACCCGAGGCATTATTTGACC | | |
| | GGATCTACACCCACCAGAGTGATGTGTGGTCTTTCGGGGTGCTCCTGTGGGAGATCTTCACTCTGGG | | |
| | CGGCTCCCCATACCCCGGTGTGCCTGTGGAGGAACTTTTCAAGCTGCTGAAGGAGGGTCACCGCATG | | |
| | GACAAGCCCAGTAACTGCACCAACGAGCTGTACATGATGATGCGGGACTGCTGGCATGCAGTGCCCT | | |
| | CACAGAGACCCACCTTCAAGCAGCTGGTGGAAGACCTGGACCGCATCGTGGCCTTGACCTCCAACCA | | |
| | GGAGTACCTGGACCTGTCCATGCCCCTGGACCAGTACTCCCCCAGCTTTCCCGACACCCGGAGCTCT | | |
| | ACGTGCTCCTCAGGGGAGGATTCCGTCTTCTCTCATGAGCCGCTGCCCGAGGAGCCCTGCCTGCCCC | | |
| | GACACCCAGCCCAGCTTGCCAATCGGGGACTCAAACGCCGCTGACTGCCACCCACACGCCCTCCCCA | | |
| | GACTCCACCGTCAGCTGTAACCCTCACCCACAGCCCCTGCTGGGCCCACCACCTGTCCGTCCCTGTC | | |
| | CCCTTTCCTGCTGGCAGCCGGCTGCCTACCAGGGGCCTTCCTGTGTGGCCTGCTTCA | | |
| | ORF Start: ATG at 116 | | ORF Stop: TGA at 2588 |
| | SEQ ID NO:4 | 824 aa | MW at 92134.0 kD |
| NOV1b, | MWSWKCLLFWAVLVTATLCTARPSPTLPEQAQPWGAPVEVESFLVHPGDLLQLRCRLRDDVQSINWL | | |
| CG101719-04 | RDGVQLAESNRTRITGEEVEVQDSVPADSGLYACVTSSPSGSDTTYFSVNVSDALPSSEDDDDDDDS | | |
| Protein | SSEEKETDNTKPNRMPVAPYWTSPEKMEKKLHAVPAAKTVKFKCPSSGTPNPTLRWLKNGKEFKPDH | | |
| Sequence | RIGGYKVRYATWSIIMDSVVPSDKGNYTCIVENEYGSINHTYQLDVVERSPHRPILQAGLPANKTVA | | |
| | LGSNVEFMCKVYSDPQPHIQWLKHIEVNGSKIGPDNLPYVQILKHSGINSSDAEVLTLFNVTEAQSG | | |
| | EYVCKVSNYIGEANQSAWLTVTRPVAKALEERPAVMTSPLYLEIIIYCTGAFLISCMVGSVIVYKMK | | |
| | SGTKKSDFHSQMAVHKLAKSIPLRRQVTVSADSSASMNSGVLLVRPSRLSSSGTPMLAGVSEYELPE | | |
| | DPRWELPRDRLVLGKPLGEGCFGQVVLAEAIGLDKDKPNRVTKVAVKMLKSDATEKDLSDLISEMEM | | |
| | MKMIGKHKNIINLLGACTQDGPLYVIVEYASKGNLREYLQARRPPGLEYCYNPSHNPEEQLSSKDLV | | |
| | SCAYQVARGMEYLASKKCIHRDLAARNVLVTEDNVMKIADFGLARDIHHIDYYKKTTNGRLPVKWMA | | |
| | PEALFDRIYTHQSDVWSFGVLLWEIFTLGGSPYPGVPVEELFKLLKEGHRMDKPSNCTNELYMMMRD | | |
| | CWHAVPSQRPTFKQLVEDLDRIVALTSNQEYLDLSMPLDQYSPSFPDTRSSTCSSGEDSVFSHEPLP | | |
| | EEPCLPRHPAQLANRGLKRR | | |
| | SEQ ID NO:5 | 2470 bp | |
| NOV1c, | CGAGGCGGAACCTCCAGCCCGAGCGAGGGTCAGTTTGAAAAGGAGGATCGAGCTCACTGTGGAGTATC | | |
| CG101719-05 | CATGGAGATGTGGAGCCTTGTCACCAACCTCTAACTGCAGAACTGGGATGTGGAGCTGGAAGTGCCTC | | |
| DNA Sequence | CTCTTCTGGGCTGTGCTGGTCACAGCCACACTCTGCACCGCTAGGCCGTCCCCGACCTTGCCTGAACA | | |
| | AGATGCTCTCCCCTCCTCGGAGGATGATGATGATGATGATGACTCCTCTTCAGAGGAGAAAGAAACAG | | |
| | ATAACACCAAACCAAACCGTATGCCCGTAGCTCCATATTGGACATCCCCAGAAAAGATGGAAAAGAAA | | |
| | TTGCATGCAGTGCCGGCTGCCAAGACAGTGAAGTTCAAATGCCCTTCCAGTGGGACCCCAAACCCCAC | | |
| | ACTGCGCTGGTTGAAAAATGGCAAAGAATTCAAACCTGACCACAGAATTGGAGGCTACAAGGTCCGTT | | |
| | ATGCCACCTGGAGCATCATAATGGACTCTGTGGTGCCCTCTGACAAGGGCAACTACACCTGCATTGTG | | |
| | GAGAATGAGTACGGCAGCATCAACCACACATACCAGCTGGATGTCGTGGAGCGGTCCCCTCACCGGCC | | |
| | CATCCTGCAAGCAGGGTTGCCCGCCAACAAAACAGTGGCCCTGGGTAGCAACGTGGAGTTCATGTGTA | | |
| | AGGTGTACAGTGACCCGCAGCCGCACATCCAGTGGCTAAAGCACATCGAGGTGAATGGGAGCAAGATT | | |
| | GGCCCAGACAACCTGCCTTATGTCCAGATCTTGAAGCATTCGGGGATTAATAGCTCGGATGCGGAGGT | | |
| | GCTGACCCTGTTCAATGTGACAGAGGCCCAGAGCGGGGAGTATGTGTGTAAGGTTTCCAATTATATTG | | |
| | GTGAAGCTAACCAGTCTGCGTGGCTCACTGTCACCAGACCTGTGGCAAAAGCCCTGGAAGAGAGGCCG | | |
| | GCAGTGATGACCTCGCCCCTGTACCTGGAGATCATCATCTATTGCACAGGGGCCTTCCTCATCTCCTG | | |
| | CATGGTGGGGTCGGTCATCGTCTACAAGATGAAGAGTGGTACCAAGAAGAGTGACTTCCACAGCCAGA | | |
| | TGGCTGTGCACAAGCTGGCCAAGAGCATCCCTCTGCGCAGACAGGTAACAGTGTCTGCTGACTCCAGT | | |
| | GCATCCATGAACTCTGGGGTTCTTCTGGTTCGGCCATCACGGCTCTCCTCCAGTGGGACTCCCATGCT | | |
| | AGCAGGGGTCTCTGAGTATGAGCTTCCCGAAGACCCTCGCTGGGAGCTGCCTCGGGACAGACTGGTCT | | |
| | TAGGCAAACCCCTGGGAGAGGGCTGCTTTGGGCAGGTGGTGTTGGCAGAGGCTATCGGGCTGGACAAG | | |
| | GACAAACCCAACCGTGTGACCAAAGTGGCTGTGAAGATGTTGAAGTCGGACGCAACAGAGAAAGACTT | | |
| | GTCAGACCTGATCTCAGAAATGGAGATGATGAAGATGATCGGGAAGCATAAGAATATCATCAACCTGC | | |
| | TGGGGGCCTGCACGCAGGATGGTCCCTTGTATGTCATCGTGGAGTATGCCTCCAAGGGCAACCTGCGG | | |
| | GAGTACCTGCAGGCCCGGAGGCCCCCAGGGCTGGAATACTGCTACAACCCCAGCCACAACCCAGAGGA | | |
| | GCAGCTCTCCTCCAAGGACCTGGTGTCCTGCGCCTACCAGGTGGCCCGAGGCATGGAGTATCTGGCCT | | |
| | CCAAGAAGTGCATACACCGAGACCTGGCAGCCAGGAATGTCCTGGTGACAGAGGACAATGTGATGAAG | | |
| | ATAGCAGACTTTGGCCTCGCACGGGACATTCACCACATCGACTACTATAAAAAGACAACCAACGGCCG | | |
| | ACTGCCTGTGAAGTGGATGGCACCCGAGGCATTATTTGACCGGATCTACACCCACCAGAGTGATGTGT | | |
| | GGTCTTTCGGGGTGCTCCTGTGGGAGATCTTCACTCTGGGCGGCTCCCCATACCCCGGTGTGCCTGTG | | |
| | GAGGAACTTTTCAAGCTGCTGAAGGAGGGTCACCGCATGGACAAGCCCAGTAACTGCACCAACGAGCT | | |
| | GTACATGATGATGCGGGACTGCTGGCATGCAGTGCCCTCACAGAGACCCACCTTCAAGCAGCTGGTGG | | |
| | AAGACCTGGACCGCATCGTGGCCTTGACCTCCAACCAGGAGTACCTGGACCTGTCCATGCCCCTGGAC | | |
| | CAGTACTCCCCCAGCTTTCCCGACACCCGGAGCTCTACGTGCTCCTCAGGGGAGGATTCCGTCTTCTC | | |
| | TCATGAGCCGCTGCCCGAGGAGCCCTGCCTGCCCCGACACCCAGCCCAGCTTGCCAATCGGGGACTCA | | |
| | AACGCCGCTGACTGCCACCCACACGCCCTCCCCAGACTCCACCGTCAGCTGTAACCCTCACCCACAGC | | |
| | CCCTGCTGGGCCCACCACCTGTCCGTCCCTGTCCCCTTTCCTGCTGGCAGCCGGCTGCCTACCAGGGG | | |
| | CCTTCCTGTGTGGCCTGCTTCA | | |
| | ORF Start: ATG at 116 | | ORF Stop: TGA at 2321 |
| | SEQ ID NO:6 | 735 aa | MW at 82428.4 kD |
| NOV1c, | MWSWKCLLFWAVLVTATLCTARPSPTLPEQDALPSSEDDDDDDDSSSEEKETDNTKPNRMPVAPYWTS | | |
| CG101719-05 | PEKMEKKLHAVPAAKTVKFKCPSSGTPNPTLRWLKNGKEFKPDHRIGGYKVRYATWSIIMDSVVPSDK | | |
| Protein | GNYTCIVENEYGSINHTYQLDVVERSPHRPILQAGLPANKTVALGSNVEFMCKVYSDPQPHIQWLKHI | | |
| Sequence | EVNGSKIGPDNLPYVQILKHSGINSSDAEVLTLFNVTEAQSGEYVCKVSNYIGEANQSAWLTVTRPVA | | |
| | KALEERPAVMTSPLYLEIIIYCTGAFLISCMVGSVIVYKMKSGTKKSDFHSQMAVHKLAKSIPLRRQV | | |
| | TVSADSSASMNSGVLLVRPSRLSSSGTPMLAGVSEYELPEDPRWELPRDRLVLGKPLGEGCFGQVVLA | | |
| | EAIGLDKDKPNRVTKVAVKMLKSDATEKDLSDLISEMEMMKMIGKHKNIINLLGACTQDGPLYVIVEY | | |
| | ASKGNLREYLQARRPPGLEYCYNPSHNPEEQLSSKDLVSCAYQVARGMEYLASKKCIHRDLAARNVLV | | |
| | TEDNVMKIADFGLARDIHHIDYYKKTTNGRLPVKWMAPEALFDRIYTHQSDVWSFGVLLWEIFTLGGS | | |
| | PYPGVPVEELFKLLKEGHRMDKPSNCTNELYMMMRGCWHAVPSQRPTFKQLVEDLDRIVALTSNQEYL | | |
| | DLSMPLDQYSPSFPDTRSSTCSSGEDSVFSHEPLPEEPCLPRHPAQLANRGLKRR | | |
| | SEQ ID NO:7 | 2731 bp | |
| NOV1d, | CGAGGCGGAACCTCCAGCCCGAGCGAGGGTCAGTTTGAAAAGGAGGATCGAGCTCACTGTGGAGTAT | | |
| CG101719-01 | CCATGGAGATGTGGAGCCTTGTCACCAACCTCTAACTGCAGAACTGGGATGTGGAGCTGGAAGTGCC | | |
| DNA Sequence | TCCTCTTCTGGGCTGTGCTGGTCACAGCCACACTCTGCACCGCTAGGCCGTCCCCGACCTTGCCTGA | | |
| | ACAAGCCCAGCCCTGGGGAGCCCCTGTGGAAGTGGAGTCCTTCCTGGTCCACCCCGGTGACCTGCTG | | |
| | CAGCTTCGCTGTCGGCTGCGGGACGATGTGCAGAGCATCAACTGGCTGCGGGACGGGGTGCAGCTGG | | |
| | CGGAAAGCAACCGCACCCGCATCACAGGGGAGGAGGTGGAGGTGCAGGACTCCGTGCCCGCAGACTC | | |
| | CGGCCTCTATGCTTGCGTAACCAGCAGCCCCTCGGGCAGTGACACCACCTACTTCTCCGTCAATGTT | | |
| | TCAGATGCTCTCCCCTCCTCGGAGGATGATGATGATGATGATGACTCCTCTTCAGAGGAGAAAGAAA | | |
| | CAGATAACACCAAACCAAACCGTATGCCCGTAGCTCCATATTGGACATCCCCAGAAAAGATGGAAAA | | |
| | GAAATTGCATGCAGTGCCGGCTGCCAAGACAGTGAAGTTCAAATGCCCTTCCAGTGGGACCCCAAAC | | |
| | CCCACACTGCGCTGGTTGAAAAATGGCAAAGAATTCAAACCTGACCACAGAATTGGAGGCTACAAGG | | |
| | TCCGTTATGCCACCTGGAGCATCATAATGGACTCTGTGGTGCCCTCTGACAAGGGCAACTACACCTG | | |
| | CATTGTGGAGAATGAGTACGGCAGCATCAACCACACATACCAGCTGGATGTCGTGGAGCGGTCCCCT | | |
| | CACCGGCCCATCCTGCAAGCAGGGTTGCCCGCCAACAAAACAGTGGCCCTGGGTAGCAACGTGGAGT | | |
| | TCATGTGTAAGGTGTACAGTGACCCGCAGCCGCACATCCAGTGGCTAAAGCACATCGAGGTGAATGG | | |
| | GAGCAAGATTGGCCCAGACAACCTGCCTTATGTCCAGATCTTGAAGACTGCTGGAGTTAATACCACC | | |
| | GACAAAGAGATGGAGGTGCTTCACTTAAGAAATGTCTCCTTTGAGGACGCAGGGGAGTATACGTGCT | | |
| | TGGCGGGTAACTCTATCGGACTCTCCCATCACTCTGCATGGTTGACCGTTCTGGAAGCCCTGGAAGA | | |
| | GAGGCCGGCAGTGATGACCTCGCCCCTGTACCTGGAGATCATCATCTATTGCACAGGGGCCTTCCTC | | |
| | ATCTCCTGCATGGTGGGGTCGGTCATCGTCTACAAGATGAAGAGTGGTACCAAGAAGAGTGACTTCC | | |
| | ACAGCCAGATGGCTGTGCACAAGCTGGCCAAGAGCATCCCTCTGCGCAGACAGGTAACAGTGTCTGC | | |
| | TGACTCCAGTGCATCCATGAACTCTGGGGTTCTTCTGGTTCGGCCATCACGGCTCTCCTCCAGTGGG | | |
| | ACTCCCATGCTAGCAGGGGTCTCTGAGTATGAGCTTCCCGAAGACCCTCGCTGGGAGCTGCCTCGGG | | |
| | ACAGACTGGTCTTAGGCAAACCCCTGGGAGAGGGCTGCTTTGGGCAGGTGGTGTTGGCAGAGGCTAT | | |
| | CGGGCTGGACAAGGACAAACCCAACCGTGTGACCAAAGTGGCTGTGAAGATGTTGAAGTCGGACGCA | | |
| | ACAGAGAAAGACTTGTCAGACCTGATCTCAGAAATGGAGATGATGAAGATGATCGGGAAGCATAAGA | | |
| | ATATCATCAACCTGCTGGGGGCCTGCACGCAGGATGGTCCCTTGTATGTCATCGTGGAGTATGCCTC | | |
| | CAAGGGCAACCTGCGGGAGTACCTGCAGGCCCGGAGGCCCCCAGGGCTGGAATACTGCTACAACCCC | | |
| | AGCCACAACCCAGAGGAGCAGCTCTCCTCCAAGGACCTGGTGTCCTGCGCCTACCAGGTGGCCCGAG | | |
| | GCATGGAGTATCTGGCCTCCAAGAAGTGCATACACCGAGACCTGGCAGCCAGGAATGTCCTGGTGAC | | |
| | AGAGGACAATGTGATGAAGATAGCAGACTTTGGCCTCGCACGGGACATTCACCACATCGACTACTAT | | |
| | AAAAAGACAACCAACGGCCGACTGCCTGTGAAGTGGATGGCACCCGAGGCATTATTTGACCGGATCT | | |
| | ACACCCACCAGAGTGATGTGTGGTCTTTCGGGGTGCTCCTGTGGGAGATCTTCACTCTGGGCGGCTC | | |
| | CCCATACCCCGGTGTGCCTGTGGAGGAACTTTTCAAGCTGCTGAAGGAGGGTCACCGCATGGACAAG | | |
| | CCCAGTAACTGCACCAACGAGCTGTACATGATGATGCGGGACTGCTGGCATGCAGTGCCCTCACAGA | | |
| | GACCCACCTTCAAGCAGCTGGTGGAAGACCTGGACCGCATCGTGGCCTTGACCTCCAACCAGGAGTA | | |
| | CCTGGACCTGTCCATGCCCCTGGACCAGTACTCCCCCAGCTTTCCCGACACCCGGAGCTCTACGTGC | | |
| | TCCTCAGGGGAGGATTCCGTCTTCTCTCATGAGCCGCTGCCCGAGGAGCCCTGCCTGCCCCGACACC | | |
| | CAGCCCAGCTTGCCAATCGGGGACTCAAACGCCGCTGACTGCCACCCACACGCCCTCCCCAGACTCC | | |
| | ACCGTCAGCTGTAACCCTCACCCACAGCCCCTGCTGGGCCCACCACCTGTCCGTCCCTGTCCCCTTT | | |
| | CCTGCTGGCAGCCGGCTGCCTACCAGGGGCCTTCCTGTGTGGCCTGCTTCA | | |
| | ORF Start: ATG at 116 | | Stop: TGA at 2582 |
| | SEQ ID NO:8 | 822 aa | MW at 91965.8 kD |
| NOV1d, | MWSWKCLLFWAVLVTATLCTARPSPTLPEQAQPWGAPVEVESFLVHPGDLLQLRCRLRDDVQSINWL | | |
| CG101719-01 | RDGVQLAESNRTRITGEEVEVQDSVPADSGLYACVTSSPSGSDTTYFSVNVSDALPSSEDDDDDDDS | | |
| Protein | SSEEKETDNTKPNRMPVAPYWTSPEKMEKKLHAVPAAKTVKFKCPSSGTPNPTLRWLKNGKEFKPDH | | |
| Sequence | RIGGYKVRYATWSIIMDSVVPSDKGNYTCIVENEYGSINHTYQLDVVERSPHRPILQAGLPANKTVA | | |
| | LGSNVEFMCKVYSDPQPHIQWLKHIEVNGSKIGPDNLPYVQILKTAGVNTTDKEMEVLHLRNVSFED | | |
| | AGEYTCLAGNSIGLSHHSAWLTVLEALEERPAVMTSPLYLEIIIYCTGAFLISCMVGSVIVYKMKSG | | |
| | TKKSDFHSQMAVHKLAKSIPLRRQVTVSADSSASMNSGVLLVRPSRLSSSGTPMLAGVSEYELPEDP | | |
| | RWELPRDRLVLGKPLGEGCEGQVVLAEAIGLDKDKPNRVTKVAVKMLKSDATEKDLSDLISEMEMMK | | |
| | MIGKHKNIINLLGACTQDGPLYVIVEYASKGNLREYLQARRPPGLEYCYNPSHNPEEQLSSKDLVSC | | |
| | AYQVARGMEYLASKKCIHRDLAARNVLVTEDNVMKIADFGLARDIHHIDYYKKTTNGRLPVKWMAPE | | |
| | ALFDRIYTHQSDVWSFGVLLWEIFTLGSSPYPGVPVEELFKLLKEGHRMDKPSNCTNELYMMMRDCW | | |
| | HAVPSQRPTFKQLVEDLDRIVALTSNQEYLDLSMPLDQYSPSFPDTRSSTCSSGEDSVFSHEPLPEE | | |
| | PCLPRHPAQLANRGLKRR | | |
| | SEQ ID NO:9 | 203 bp | |
| NOV1e, | ATGTGGAGCTGGAAGTGCCTCCTCTTCTGGGCTGTGCTGGTCACAGCCACACTCTGCACCGCTAGGCC | | |
| CG101719-03 | GTCCCCGACCTTGCCTGAACAAGCTTGCCCAGATCTCCAGGAGGCTAAGTGGTGCTCGGCCAGCTTCC | | |
| DNA Sequence | ACTCCATCACTCCCTTGCCATTTGGACTTGGTACTCGGCTTAGTGATATGAGGCCCTGAACAGGTGG | | |
| | ORF Start: ATG at 1 | | ORF Stop: TAG at 184 |
| | SEQ ID NO:10 | 61 aa | MW at 6780.8 kD |

| | |
|---|---|
| NOV1e, | MWSWKCLLFWAVLVTATLCTARPSPTLPEQACPDLQEAKWCSASFHSITPLPFGLGTRLSD |
| CG101719-03 | |
| Protein | |
| Sequence | |

Sequence comparison of the above protein sequences yields the following sequence relationships shown in Table 1B.

TABLE 1B
Comparison of NOV1a against NOV1b through NOV1e.

| | NOV1a Residues/ | Identities/Similarities |
|---|---|---|
| Protein Sequence | Match Residues | for the Matched Region |
| NOV1b | 1 . . . 119 | 118/119 (99%) |
| | 1 . . . 119 | 118/119 (99%) |
| NOV1c | 1 . . . 118 | 50/158 (31%) |
| | 1 . . . 158 | 70/158 (43%) |
| NOV1d | 1 . . . 119 | 118/119 (99%) |
| | 1 . . . 119 | 118/119 (99%) |
| NOV1e | 1 . . . 33 | 31/33 (93%) |
| | 1 . . . 33 | 31/33 (93%) |

Further analysis of the NOV1a protein yielded the following properties shown in Table 1C.

TABLE 1C
Protein Sequence Properties NOV1a

| | |
|---|---|
| SignalP analysis: | Cleavage site between residues 24 and 25 |
| SORT II analysis: | PSG: a new signal peptide prediction method |
| | N-region: length 5; pos.chg 1; neg.chg 0 |
| | H-region: length 16; peak value 9.41 |
| | PSG score: 5.01 |
| | GvH: von Heijne's method for signal seq. recognition |
| | GvH score (threshold: -2.1): 1.64 |
| | possible cleavage site: between 21 and 22 |
| | >>> Seems to have a cleavable signal peptide (1 to 21) |
| | ALOM: Klein et al's method for TM region allocation |
| | Init position for calculation: 22 |
| | Tentative number of TMS(s) for the threshold 0.5: 0 |
| | number of TMS(s) . . . fixed |
| | PERIPHERAL Likelihood = 8.43 (at 130) |
| | ALOM score: 8.43 (number of TMSs: 0) |
| | MTOP: Prediction of membrane topology (Harmnann et al.) |
| | Center position for calculation: 10 |
| | Charge difference: -2.0 C(0.0) - N(2.0) |
| | N >= C: N-terminal side will be inside |
| | MITDISC: discrimination of mitochondrial targeting seq |

| | | | |
|---|---|---|---|
| R content: | 1 | Hyd Moment(75): | 6.11 |
| Hyd Monent(95): | 4.64 | G content: | 0 |
| D/E content: | 1 | S/T content: | 6 |
| Score: | −2.45 | | |

Gavel: prediction of cleavage sites for mitochondrial preseq
R-2 motif at 32 ARP|SP
NUCDISC: discrimination of nuclear localization signals
pat4: none
pat7: none
bipartite: none
content of basic residues: 6.7%
NLS Score: -0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
22.2%: extracellular, including cell wall
22.2%: Golgi
22.2%: vacuolar
22.2%: endoplasmic reticulum
11.1%: mitochondrial
>> prediction for CG101719-02 is exc (k = 9)

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG79680 | Tumour involved gene (TIG) | 1 . . . 119 | 118/119 (99%) | 1e−65 |
| | splice variant protein, NV-11 - | 1 . . . 119 | 118/119 (99%) | |
| | Homo sapiens, 702 aa. | | | |
| | [US2002086384-A1, | | | |
| | 04-JUL-2002] | | | |
| AAB84383 | Amino acid sequence of a | 1 . . . 119 | 118/119 (99%) | 1e−65 |
| | fibroblast growth factor | 1 . . . 119 | 118/119 (99%) | |
| | receptor - Homo sapiens, 820 | | | |
| | aa. [US6255454-B1, | | | |
| | 03-JUL-2001] | | | |
| AAY97170 | Human FGF-RI Extracellular | 1 . . . 119 | 118/119 (99%) | 1e−65 |
| | domain-Ig Fc fusion protein 1 - | 1 . . . 119 | 118/119 (99%) | |
| | Homo sapiens, 622 aa. | | | |
| | [WO200046380-A2, | | | |
| | 10-AUG-2000] | | | |
| AAY06458 | Fibroblast growth factor | 1 . . . 119 | 118/119 (99%) | 1e−65 |
| | receptor 1 - Homo sapiens, | 1 . . . 119 | 118/119 (99%) | |
| | 820 aa. [WO9935159-A1, | | | |
| | 15-JUL-1999] | | | |
| AAR47233 | Human fibroblast growth | 1 . . . 119 | 118/119 (99%) | 1e−65 |
| | factor receptor - Homo sapiens, 820 | 1 . . . 119 | 118/119 (99%) | |
| | aa. [WO9403620-A, | | | |
| | 17-FEB-1994] | | | |

In a BLAST search of public sequence datbases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| A40862 | fibroblast growth factor | 1 . . . 150 | 149/150 (99%) | 2e−85 |
| | receptor 1, secreted form - | 1 . . . 150 | 149/150 (99%) | |
| | human, 150 aa. | | | |
| C40862 | heparin-binding growth factor | 1 . . . 119 | 118/119 (99%) | 4e−65 |
| | receptor variant alpha-a2 - | 1 . . . 119 | 118/119 (99%) | |
| | human, 662 aa. | | | |
| AAH15035 | Similar to fibroblast growth | 1 . . . 119 | 118/119 (99%) | 4e−65 |
| | factor receptor 1 (fms-related | 1 . . . 119 | 118/119 (99%) | |
| | tyrosine kinase 2, Pfeiffer | | | |
| | syndrome) - Homo sapiens | | | |
| | (Human), 820 aa. | | | |
| Q8N685 | Similar to fibroblast growth | 1 . . . 119 | 118/119 (99%) | 4e−65 |
| | factor receptor 1 (fms-related | 1 . . . 119 | 118/119 (99%) | |
| | tyrosine kinase 2, | | | |
| | Pfeiffer syndrome) - Homo sapiens | | | |
| | (Human), 820 aa. | | | |
| P11362 | Basic fibroblast growth factor | 1 . . . 119 | 118/119 (99%) | 4e−65 |
| | receptor 1 precursor (EC | 1 . . . 119 | 118/119 (99%) | |
| | 2.7.1.112) (FGFR-1) (bFGF-R) | | | |
| | (Fms-like tyrosine | | | |
| | kinase-2) (c-fgr) - | | | |
| | Homo sapiens (Human), 822 aa. | | | |

PFam analysis predicts that the NOV1a protein contains the domains shown in the Table 1F.

TABLE 1F
Domain Analysis of NOV1a

| | | Identities/ | |
|---|---|---|---|
| | NOV1a | Similarities for the | |
| Pfam Domain | Match Region | Matched Region | Expect Value |
| ig | 48 . . . 103 | 14/60 (23%) | 2.1e−06 |
| | | 39/60 (65%) | |

### Example 2

The NOV2 clone was analyzed, and the nucleotide and encoded polypeptide sequences are shown in Table 2A.

TABLE 2A
NOV2 Sequence Analysis

| | SEQ ID NO:11 | 937 bp | |
|---|---|---|---|
| NOV2a, | CGCGGCCCCAGGGCTCACTTGGCGCTGAGAACGCGGGTGCAGCGTGTGATCGTCCGTGCGTCTAGCCT | | |
| CG102006-01 | | | |
| DNA Sequence | TTGCCCACGCAGCTTTCAGTCATGGCCTCCGGTAACGCGCGCATCGGAAAGCCAGCCCCTGACTTCAA | | |
| | GGCCACAGCGGTGGTTGATGGCGCCTTCAAAGAGGTGAAGCTGTCGGACTACAAAGGGAAGTACGTGG | | |
| | TCCTCTTTTTCTACCCTCTGGACTTCACTTTTGTGTGCCCCACCGAGATCATCGCGTTCACAACCGTG | | |
| | AAGAGGACTTCCGCAAAGCTGGGCTGTGAAGTGCTGGGCGTCTCGGTGGACTCTCAGTTCACCCACCT | | |
| | GGCTTGGATCAACACCCCCCGGAAAGAGGGAGGCTTGGGCCCCTTGAACATCCCCCTGCTTGCTGACG | | |
| | TGACCAGACGCTTGTCTGAGGATTACGGCGTGCTGAAAAACGATGAGGGCATTGCTTACAGGGGCCTC | | |
| | TTTATCATCGATGGCAAGGGTGTCCTTCGCCAGATCACTGTTAATGATTTGCCTGTGGGACGCTCCGT | | |
| | GGATGAGGCTCTGCGGCTGGTCCAGGCCTTCCAGTACACAGACGAGCATGGGGAAGTTTGTCCGGCTG | | |
| | CTTGGAAGCCTGGACGTGACACGATTAAGCCGAACGTGGATGACAGCAAGGAATATTTCTCCAAACAC | | |
| | AATTAGGCTGGCTAACGGATAGTGAGCTTGTGCCCCTGCCTAGGTGCCTGTGCTGGGTGTCCACCTGT | | |
| | GCCCCCACCTGGGTGCCCTATGCTGACCCAGGAAAGGCCAGACCTGCCCCTCCAAAATCCACAGTATG | | |
| | GGACCCTGGAGGGCTAGCAAGGCCTTCTCATGCCTCCACCTAGAAGCTGAATAGTGACGCCCTCCCCC | | |
| | AAGCCCACCCAGCCGCACACAGGCCTAGAGGTAACCAATAAAGTATTAGGGCC | | |
| | ORF Start: ATG at 90 | | ORF Stop: TAG at 684 |
| | SEQ ID NO:12 | 198 aa | MW at 21856.8 kD |
| NOV2a, | MASGNARIGKPAPDFKATAVVDGAFKEVKLSDYKGKYVVLFFYPLDFTFVCPTEIIAFTTVKRTSAKL | | |
| CG102006-01 | | | |
| Protein | GCEVLGVSVDSQFTHLAWINTPRKEGGLGPLNIPLLADVTRRLSEDYGVLKNDEGIAYRGLFIIDGKG | | |
| Sequence | | | |
| | VLRQITVNDLPVGRSVDEALRLVQAFQYTDEHGEVCPAAWKPGRDTIKPNVDDSKEYFSKHN | | |
| | SEQ ID NO:13 | 656 bp | |
| NOV2b, | CTCACTTGGCGCTGAGAACGCGGGTCCACGCGTGTGATCGTCCGTGCGTCTAGCCTTTGCCCACGCA | | |
| CG102006-02 | | | |
| DNA Sequence | GCTTTCAGTCATGGCCTCCGGTAACGCGCGCATCGGAAAGCCAGCCCCTGACTTCAAGGCCACAGCG | | |
| | GTGGTTGATGGCGCCTTCAAAGAGGTGAAGCTGTCGGACTACAAAGGGAAGTACGTGGTCCTCTTTT | | |
| | TCTACCCTCTGGACTTCACTTTTGTGTGCCCCACCGAGATCATCGCGTTCAGCAACCGTGCAGAGGA | | |
| | CTTCCGCAAGCTGGGCTGTGAAGTGCTGGGCGTCTCGGTGGACTCTCAGTTCACCCACCTGGCTTGG | | |
| | TATGAGCAGGGGCCAAAGAGGGAGGTTGCAGCTAAGCTCACACCCTCAGGTCCTAGCAGTGTGGCTT | | |
| | CGTGGCCATTGCTCAACCTCTGGAACCTGCGTTTCCCCATCGTGAAAATAATGGAAACATTGCCGCC | | |
| | CAAGTCTTTAAGGATGATGACAGTAATTAGCATTTGACAACTAGTTGCCTGGTATATAGAGTTGCAG | | |
| | ATGCAACTCAGATGCAACTCTATCTACTCTATGTACTTAGTTCCCAGGAGGGAGGCTGTGCTGCCCT | | |
| | ATTTCATGAAGATGGAAACTCCAGTTCACCGAAGTGAAGGGCTGTACCCATGA | | |
| | ORF Start: ATG at 78 | | ORF Stop: TGA at 504 |
| | SEQ ID NO:14 | 142 aa | MW at 15818.3 kD |

| NOV2b, | MASGNARIGKPAPDFKATAVVDGAFKEVKLSDYKGKYVVLFFYPLDFTFVCPTEIIAFSNRAEDFRK | |
|---|---|---|
| CG102006-02 | | |
| Protein | LGCEVLGVSVDSQFTHLAWYEQGPKREVAAKLTPSGPSSVASWPLLNLWNLRFPIVKIMETLPPKSL | |
| Sequence | | |
| | RMMTVISI | |
| | SEQ ID NO:15 | 923 bp |

| NOV2c, | GGCACGAGGCGCGGGTCCACGCGTGTGATCGTCCGTGCGTCTAGCCTTTGCCCACGCAGCTTTCAGTC | | |
|---|---|---|---|
| CG102006-03 | | | |
| DNA Sequence | ATGGCCTCCGGTAACGCGCGCATCGGAAAGCCAGCCCCTGACTTCAAGGCCACAGCGGTGGTTGATGG | | |
| | CGCCTTCAAAGAGGTGAAGCTGTCGGACTACAAAGGGAAGTACGTGGTCCTCTTTTTCTACCCTCTGG | | |
| | ACTTCACTTTTGTGTGCCCCACCGAGATCATCGCGTTCAGCAACCGTGCAGAGGACTTCCGCAAGCTG | | |
| | GGCTGTGAAGTGCTGGCCGTCTCGGTGGACTCTCAGTTCACCCACCTGGCTTGGATCAACACCCCCCG | | |
| | GAAAGAGGGAGGCTTGGGCCCCCTGAACATCCCCCTGCTTGCTGACGTGACCAGACGCTTGTCTGAGG | | |
| | ATTACGGCGTGCTGAAAACAGATGAGGGCATTGCCTACAGGGGCCTCTTTATCATCGATGGCAAGGGT | | |
| | GTCCTTCGCCAGATCACTGTTAATGATTTGCCTGTGGGACGCTCCGTGGATGAGGCTCTGCGGCTGGT | | |
| | CCAGGCCTTCCAGTACACAGACGAGCATGGGGAAGTTTGTCCCGCTGGCTGGAAGCCTGGCAGTGACA | | |
| | CGATTAAGCCCAACGTGGATGACAGCAAGGAATATTTCTCCAAACACAATTAGGCTGGCTAACGGATA | | |
| | GTGAGCTTGTGCCCCTGCCTAGGTGCCTGTGCTGGGTGTCCACCTGTGCCCCCACCTGGGTGCCCTAT | | |
| | GCTGACCCAGGAAAGGCCAGACCTGCCCCTCCAAACTCCACAGTATGGGACCCTGGAGGGCTACGCCA | | |
| | AGGCCTTCTCATGCCTCCACCTAGAAGCTGAATAGTGACGCCCTCCCCCAAGCCCACCCAGCCGCACA | | |
| | CAGGCCTAGAGGTAACCAATAAAGTATTAGGGAAAGGTG | | |
| | ORF Start: ATG at 69 | | ORF Stop: TAG at 663 |
| | SEQ ID NO:16 | 198 aa | MW at 21891.7 kD |

| | |
|---|---|
| NOV2c, | MASGNARIGKPAPDFKATAVVDGAFKEVKLSDYKGKYVVLFFYPLDFTFVCPTEIIAFSNRAEDFRKL |
| CG102006-03 | |
| Protein | GCEVLGVSVDSQFTHLAWINTPRKEGGLGPLNIPLLADVTRRLSEDYGVLKTDEGIAYRGLFIIDGKG |
| Sequence | |
| | VLRQITVNDLPVGRSVDEALRLVQAFQYTDEHGEVCPAGWKPGSDTIKPNVDDSKEYFSKHN |

Sequence comparison of the above protein sequences yields the following sequence relationships shown in Table 2B.

TABLE 2B
Comparison of NOV2a against NOV2b and NOV2c.

| | NOV2a Residues/ | Identities/Similarities |
|---|---|---|
| Protein Sequence | Match Residues | for the Matched Region |
| NOV2b | 1 . . . 93 | 80/94 (85%) |
| | 1 . . . 94 | 83/94 (88%) |
| NOV2c | 1 . . . 198 | 187/198 (94%) |
| | 1 . . . 198 | 188/198 (94%) |

Further analysis of the NOV2a protein yielded the following properties shown in Table 2C.

TABLE 2C
Protein Sequence Properties NOV2a

| SignalP analysis: | No Known Signal Sequence Predicted | | | |
|---|---|---|---|---|
| PSORT II | PSG: a new signal peptide prediction method | | | |
| analysis: | N-region: length 10; pos.chg 2; neg.chg 0 | | | |
| | H-region: length 3; peak value −5.37 | | | |
| | PSG score: −9.77 | | | |
| | GvH: von Heijne's method for signal seq. recognition | | | |
| | GvH score (threshold: −2.1): −5.82 | | | |
| | possible cleavage site: between 57 and 58 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 1.96 (at 37) | | | |
| | ALOM score: 1.96 (number of TMSs: 0) | | | |
| | MITDISC: discrimination of mitochondrial targeting | | | |
| | seq | | | |
| | R content: | 1 | Hyd Moment(75): | 3.10 |
| | Hyd Moment(95): | 5.71 | G content: | 2 |
| | DIE content: | 2 | S/T content: | 2 |
| | Score: | −6.95 | | |

Gavel: prediction of cleavage sites for mitochondrial
preseq R-2 motif at 17 ARI|GK
NUCDISC: discrimination of nuclear localization
signals
pat4: none
pat7: none
bipartite: none
content of basic residues: 12.6%
NLS Score: −0.47
KDEL: ER retention motif in the C-terminus: none
ER Membrane Retention Signals:
KKXX-like motif in the C-terminus: FSKH
SKL: peroxisomal targeting signal in the
C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern : none
Prenylation motif: none
menYQRL: transport motif from cell surface to Golgi:
none
Tyrosines in the tail: none
Dileucine motif in the tail: none
checking 63 PROSITE DNA binding motifs: none
checking 71 PROSITE ribosonal protein motifs: none
checking 33 PROSITE prokaryotic DNA binding
motifs: none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
Final Results (k = 9/23)
52.2%: cytoplasmic
21.7%: nuclear
17.4%: mitochondrial
8.7%: peroxisomal
>> prediction for CG102006-01 is cyt (k = 23)

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB68036 | Amino acid sequence of the | 1 . . . 198 | 187/198 (94%) | e−106 |
| | acid form of peroxyredoxin | 1 . . . 198 | 188/198 (94%) | |
| | TDX1 - Homo sapiens, 198 | | | |
| | aa. [FR2798672-A1, | | | |
| | 23-MAR-2001] | | | |
| ABP53045 | Rat thiol-specific antioxidant | 1 . . . 198 | 178/198 (89%) | e−102 |
| | (TSA) protein SEQ ID NO: 28 - | 1 . . . 198 | 184/198 (92%) | |
| | Rattus norvegicus, 198 aa. | | | |
| | [WO200264169-A1, | | | |
| | 22-AUG-2002] | | | |
| AAU78580 | Mouse peroxiredoxin II-1 | 1 . . . 198 | 176/198 (88%) | e−100 |
| | (PrxII-1) protein - Mus sp, | 1 . . . 198 | 182/198 (91%) | |
| | 198 aa. [KR99066020-A, | | | |
| | 16-AUG-1999] | | | |
| ABG26215 | Novel human diagnostic | 22 . . . 198 | 164/177 (92%) | 3e−93 |
| | protein #26206 - Homo | 43 . . . 219 | 166/177 (93%) | |
| | sapiens, 219 aa. | | | |
| | [WO200175067-A2, | | | |
| | 11-OCT-2001] | | | |
| AAW09794 | Natural killer cell enhancing | 1 . . . 198 | 166/198 (83%) | 2e−89 |
| | factor B - Homo sapiens, 178 | 1 . . . 178 | 167/198 (83%) | |
| | aa. [US5610286-A, | | | |
| | 11-MAR-1997] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P32119 | Peroxiredoxin 2 (EC1.11.1.-) | 1 . . . 198 | 187/198 (94%) | e−106 |
| | (Thioredoxin peroxidase 1) | 1 . . . 198 | 188/198 (94%) | |
| | (Thioredoxin-dependent | | | |
| | peroxide reductase 1) | | | |
| | (Thiol-specific antioxidant | | | |
| | protein) (TSA) (PRP) (Natural | | | |
| | killer cell enhancing factor B) | | | |
| | (NKEF-B) - Homo sapiens | | | |
| | (Human), 198 aa. | | | |
| 168897 | probable thioredoxin | 1 . . . 198 | 185/198 (93%) | e−105 |
| | peroxidase (EC 1.11.1.-) 1- | 1 . . . 198 | 186/198 (93%) | |
| | human, 198 aa. | | | |
| Q8K3U7 | Peroxiredoxin 2 - Cricetulus | 1 . . . 198 | 180/198 (90%) | e−102 |
| | griseus (Chinese hamster), 198 | 1 . . . 198 | 184/198 (92%) | |
| | aa. | | | |
| P35704 | Peroxiredoxin 2 (EC 1.11.1.-) | 1 . . . 198 | 178/198 (89%) | e−102 |
| | (Thioredoxin peroxidase 1) | 1 . . . 198 | 184/198 (92%) | |
| | (Thioredoxin-dependent | | | |
| | peroxide reductase 1) | | | |
| | (Thiol-specific antioxidant | | | |
| | protein) (TSA) - Rattus | | | |
| | norvegicus (Rat), 198 aa. | | | |
| Q61171 | Peroxiredoxin 2(EC 1.11.1.-) | 1 . . . 198 | 178/198 (89%) | e−101 |
| | (Thioredoxin peroxidase 1) | 1 . . . 198 | 185/198 (92%) | |
| | (Thioredoxin-dependent | | | |
| | peroxide reductase 1) | | | |
| | (Thiol-specific antioxidant | | | |
| | protein) (TSA) - Mus | | | |
| | musculus (Mouse), 198 aa. | | | |

PFam analysis predicts that the NOV2a protein contains the domains shown in the Table 2F.

TABLE 2F
Domain Analysis of NOV2a

| | | Identities/ | |
|---|---|---|---|
| | NOV2a | Similarities | |
| Pfam | Match | for the | Expect |
| Domain | Region | Matched Region | Value |
| AhpC-TSA | 8 . . . 157 | 94/161 (58%) | 1.3e−80 |
| | | 139/161 (86%) | |

### Example 3

The NOV3 clone was analyzed, and the nucleotide and encoded polypeptide sequences are shown in Table 3A.

TABLE 3A
NOV3 Sequence Analysis

| | SEQ ID NO:17 | 1485 bp | |
|---|---|---|---|
| NOV3a, | ACCATGGGCCACCATCACCACCATCACGACTCATCTGTCATTCAAAGGAAAAAAGTAGCTGTCATTGG | | |
| CG127322-07 | | | |
| DNA Sequence | TGGTGGCTTGGTTGGTTCATTACAAGCATGCTTTCTTGCAAAGAGGAATTTCCAGATTGATGTATATG | | |
| | AAGCTAGGGAAGATACTCGAGTGGCTACCTTCACACGTGGAAGAAGCATTAACTTAGCCCTTTCTCAT | | |
| | AGAGGACGACAAGCCTTGAAAGCTGTTGGCCTGGAAGATCAGATTGTATCCCAAGGTATTCCCATGAG | | |
| | AGCAAGAATGATCCACTCTCTTTCAGGAAAAAAGTCTGCAATTCCCTATGGGACAAAGTCTCAGTATA | | |
| | TTCTTTCTGTAAGCAGAGAAAATCTAAACAAGGATCTATTGACTGCTGCTGAGAAATACCCCAATGTG | | |
| | AAAATGCACTTTAACCACAGGCTGTTGAAATGTAATCCAGAGGAAGGAATGATCACAGTGCTTGGATC | | |
| | TGACAAAGTTCCCAAAGATGTCACTTGTGACCTCATTGTAGGATGTGATGGAGCCTATTCAACTGTCA | | |
| | GATCTCACCTGATGAACAAACCTCGCTTTGATTACAGTCAGCAGTACATTCCTCATGGGTACATGGAG | | |
| | TTGACTATTCCACCTAAGAACGGAGATTATGCCATGGAACCTAATTATCTGCATATTTGGCCTAGAAA | | |
| | TACCTTTATGATGATTGCACTTCCTAACATGAACAAATCATTCACATGTACTTTGTTCATGGCCTTTG | | |
| | AAGAGTTTGAAAAACTTCTAACCAGTAATGATGTGGTAGATTTCTTCCAGAAATACTTTCCGGATGCC | | |
| | ATCCCTCTAATTGGAGAGAAACTCCTAGTGCAAGATTTCTTCCTGTTGCCTGCCCAGCCCATGATATC | | |
| | TGTAAAGTGCTCTTCATTTCACTTTAAATCTCACTGTGTACTGCTGGGAGATGCAGCTCATGCTATAG | | |
| | TGCCGTTTTTTGGGCAAGGAATGAATGCGGGCTTTGAAGACTGCTTGGTATTTGATGAGTTAATGGAT | | |
| | AAATTCAGTAACGACCTTAGTTTGTGTCTTCCTGTGTTCTCAAGATTGAGAATCCCAGATGATCACGC | | |
| | GATTTCAGACCTATCCATGTACAATTACATAGAGATGCGAGCACATGTCAACTCAAGCTGGTTCATTT | | |
| | TTCAGAAGAACATGGAGAGATTTCTTCATGCGATTATGCCATCGACCTTTATCCCTCTCTATACAATG | | |
| | GTCACTTTTTCCAGAATAAGATACCATGAGGCTGTGCAGCGTTGGCATTGGCAAAAAAAGGTGATAAA | | |
| | CAAAGGACTCTTTTTCTTGGGATCACTGATAGCCATCAGCAGTACCTACCTACTTATACACTACATGT | | |
| | CACCACGATCTTTCCTCCGCTTGAGAAGACCATGGAACTGGATAGCTCACTTCCGGAATACAACATGT | | |
| | TTCCCCGCAAAGGCCGTGGACTCCCTAGAACAAATTTCCAATCTCATTAGCAGGTGA | | |
| | ORF Start: at 1 | | ORF Stop: TGA at 1483 |
| | SEQ ID NO: 18 | 494 aa | MW at 56790.3 kD |
| NOV3a, | TMGHHHHHHDSSVIQRKKVAVIGGGLVGSLQACFLAKRNFQIDVYEAREDTRVATFTRGRSINLALSH | | |
| CG127322-07 | | | |
| Protein | RGRQALKAVGLEDQIVSQGIPMRARMIHSLSGKKSAIPYGTKSQYILSVSRENLNKDLLTAAEKYPNV | | |
| Sequence | | | |
| | KMHFNHRLLKCNPEEGMITVLGSDKVPKDVTCDLIVGCDGAYSTVRSHLMKKPRFDYSQQYIPHGYME | | |
| | LTIPPKNGDYAMEPNYLHIWPRNTFMMIALPNMNKSFTCTLFMPFEEFEKLLTSNDVVDFFQKYFPDA | | |
| | IPLIGEKLLVQDFFLLPAQPMISVKCSSFHFKSHCVLLGDAAHAIVPFFGQGMNAGFEDCLVFDELMD | | |
| | KFSNDLSLCLPVFSRLRIPDDHAISDLSMYNYIEMRAHVNSSWFIFQKNMERFLHAIMPSTFIPLYTM | | |
| | VTFSRIRYHEAVQRWHWQKKVINKGLFFLGSLIAISSTYLLIHYMSPRSFLRLRRPWNWIAHFRNTTC | | |
| | FPAKAVDSLEQISNLISR | | |
| | SEQ ID NO: 19 | 5000 bp | |
| NOV3b, | GGCACGAGCAGAAGCAACAATAATTGTGAAAAATACTTCAGCAGTTATGGACTCATCTGTCATTCAAA | | |
| CG127322-01 | | | |
| DNA Sequence | GGAAAAAAGTAGCTGTCATTGGTGGTGGCTTGGTTGGCTCATTACAAGCATGCTTTCTTGCAAAGAGG | | |
| | AATTTCCAGATTGATGTATATGAAGCTAGGGAAGATACTCGAGTGGCTACCTTCACACGTGGAAGAAG | | |
| | CATTAACTTAGCCCTTTCTCATAGAGGACGACAAGCCTTGAAAGCTGTTGGCCTGGAAGATCAGATTG | | |
| | TATCCCAAGGTATTCCCATGAGAGCAAGAATGATCCACTCTCTTTCAGGAAAAAAGTCTGCAATTCCC | | |
| | TATGGGACAAAGTCTCAGTATATTCTTTCTGTAAGCAGAGAAAATCTAAACAAGGATCTATTGACTGC | | |
| | TGCTGAGAAATACCCCAATGTGAAAATGCACTTTAACCACAGGCTGTTGAAATGTAATCCAGAGGAAG | | |
| | GAATGATCACAGTGCTTGGATCTGACAAAGTTCCCAAAGATGTCACTTGTGACCTCATTGTAGGATGT | | |
| | GATGGAGCCTATTCAACTGTCAGATCTCACCTGATGAAGAAACCTCGCTTTGATTACAGTCAGCAGTA | | |
| | CATTCCTCATGGGTACATGGAGTTGACTATTCCACCTAAGAACGGAGATTATGCCATGGAACCTAATT | | |
| | ATCTGCATATTTGGCCTAGAAATACCTTTATGATGATTGCACTTCCTAACATGAACAAATCATTCACA | | |
| | TGTACTTTGTTCATGCCCTTTGAAGAGTTTGAAAAACTTCTAACCAGTAATGATGTGGTAGATTTCTT | | |
| | CCAGAAATACTTTCCGGATGCCATCCCTCTAATTGGAGAGAAACTCCTAGTGCAAGATTTCTTCCTGT | | |
| | TGCCTGCCCAGCCCATGATATCTGTAAAGTGCTCTTCATTTCACTTTAAATCTCACTGTGTACTGCTG | | |
| | GGAGATGCAGCTCATGCTATAGTGCCGTTTTTTGGGCAAGGAATGAATGCGGGCTTTGAAGACTGCTT | | |
| | GGTATTTGATGAGTTAATGGATAAATTCAGTAACGACCTTAGTTTGTGTCTTCCTGTGTTCTCAAGAT | | |
| | TGAGAATCCCAGATGATCACGCGATTTCAGACCTATCCATGTACAATTACATAGAGATGCGAGCACAT | | |
| | GTCAACTCAAGCTGGTTCATTTTTCAGAAGAACATGGAGAGATTTCTTCATGCGATTATGCCATCGAC | | |
| | CTTTATCCCTCTCTATACAATGGTCACTTTTTCCAGAATAAGATACCATGAGGCTGTGCAGCGTTGGC | | |
| | ATTGGCAAAAAAAGGTGATAAACAAAGGACTCTTTTTCTTGGGATCACTGATAGCCATCAGCAGTACC | | |
| | TACCTACTTATACACTACATGTCACCACGATCTTTCCTCTGCTTGAGAAGACCATGGAACTGGATAGC | | |
| | TCACTTCCGGAATACAACATGTTTCCCCGCAAAGGCCGTGGACTCCCTAGAACAAATTTCCAATCTCA | | |
| | TTAGCAGGTGATAGAAAGGTTTTGTGGTAGCAAATGCATGATTTCTCTGTGACCAAAATTAAGCATGA | | |
| | AAAAAATGTTTCCATTGCCATATTTGATTCACTAGTGGAAGATAGTGTTCTGCTTATAATTAAACTGA | | |
| | ATGTAGAGTATCTCTGTATGTTAATTGCAATTACTGGTTGGGGGGTGCATTTTAAAAGATGAAACATG | | |
| | CAGCTTCCCTACATTACACACACTCAGGTTGAGTCATTCTAACTATAAAAGTGCAATGACTAAGATCC | | |
| | TTCACTTCTCTGAAAGTAAGGCCCTAGATGCCTCAGGGAAGACAGTAATCATGCCTTTTCTTTAAAAG | | |
| | ACACAATAGGACTCGCAACAGCATTGACTCAACACCTAGGACTAAAAATCACAACTTAACTAGCATGT | | |
| | TAACTGCACTTTTCATTACGTGAATGGAACTTACCTAACCACAGGGCTCAGACTTACTAGATAAAACC | | |
| | AGAAATGGAAATAAGGAATTCAGGGGACTTCCAGAGACTTACAAAATGAACTCATTTTATTTTCCCAC | | |
| | CTTCAAATATAAGTATTATCATCTATCTGTTTATCGTCTATCTATCTATCATCTATCTATCTATCTAT | | |
| | CATCTATCTATCTATCTATCTATCTATCTATCTATCTATCTATCTATCTATCTCTATTTATTTATGTA | | |
| | TTTAGAGATCAGGTCTCACTCTGTTGACCAGGCTGGAGTGCAGTGGTGAGATCTGGGTTCACTGCAAC | | |
| | CTCTGCCTCCTGGGCTCAAGCAATCCTCCCACTTCAGCCTCCCAAATAGCTGGGGCTACCATGGTATT | | |
| | TTTCAGTAGAGACCGGGTCTTGCCATGCTGCCCAGGCCAGTCTCAAACTCCTGGCCTCATGTGATCTG | | |
| | CCCACCTCAGCCTCCCAAAGTACAGGGATTAGAGTTGTGAGCCACCGCTGCCAGCCCAGAGTTACCCT | | |
| | CTAAAGATAAGAAAAAGGCTATTAATATCATACTAAGTGAAGGACAGGAAAGGGTTTTATTCATAAAT | | |
| | TAAATGTCTACATGTGCCAGAATGGAAAGGAAACAAGGGGAGACAACTTTTATAGAAATACAAAGCCA | | |
| | TTACTTTATTCAATTTCAGACCCTCAGAAGCAATTTACTAATTTATTCTTCGACTACATACTGCAGCA | | |
| | GAACCAGCAATACACTTGATTTTTAAAAGCACATTTAGTGAAATGTTTTCTTTGGTTCATCCTTCTTT | | |
| | AACAGGCTGCTGAGTCACTCAGAAATCCTTCAAACATGATTAATTATGAAGATGAAACACTAGAGTCA | | |
| | TATAAGAAATAAAAATTGGGCAATAAAATAAAATGATTCAGTGTTTCTTTTCTATATTGTCAATGAAA | | |
| | ACCTTGAGTTCTAATAATCCATGTTCAGTTTGTAGGGAAAGAAAAAATAATTTTTCCTTCTACCCACT | | |
| | TTAGGTTCCTTGGCTGGGGCCCCTATAACAAAAGACAGATTGACAAGAGAAAAACAAACATAAATTTA | | |
| | TTAGCGGGTATATGTAATATATATGTGGGAAATACAGGGGAATGAGCAAATCTCAAAGAGCTGGCGTC | | |
| | TTAGAACTCCCTGGCTTATATAGCATCGACAAAGAACAGTAAATTTTTAGAGAAACAACAAAACAAAG | | |
| | AAAAAGAGCTTTGAGTCTGTAGGGGCAGCAATTTGGGGGAAGCAAATATATGGGAGTTTGCCTTGTAG | | |
| | ATTCCTCTGGTGGTGGTCTCCAGGCTGACAAGGATTCAAAGTTGTCTCTGAAACTCCTCTTTGTCATA | | |
| | CTGCACATATAAAACGTCTTTTGTTTCCAACAAGAGGATTTCTTTTTCATTCTAGAATTATCTCCTTG | | |
| | ATAACTTGATCAGATATAGGACATGACACTGAATAGAGTCCAACAGTACAAAAAAAATTCAGTATGTT | | |
| | CTAGCTACTTCACACATGTGTACGCGACAGTTATTTTTACAGTAAGGTATTTTCGAGAAAAATGCATT | | |
| | ACGTGTTTTGGAAAATAGAGTAATTTAAAAAATATATTTGAAATGAAAATCTCCAACACATTAGAAGA | | |
| | TGATGATGTTAGATGCCCATCGTGTGCCACAAGTGGTTTTTTCATTATGTAAAGCACCCGTTGAATTA | | |
| | AAAGAATTTGTTTTTGTTCAACCTCTTCCTGAGGCCCAAGAGCATATGGGCAATTCGGATTTCCTGCT | | |
| | GGACCACAAGGTTCTGTTGATATTACATAGAAACGGGTATTCCAGACACTTCTTATGATGAAAGTCCA | | |
| | AAAGTGGCATCCAATTTAAGGCCCCATCTTTCGTTGCCATTCTTCATTCCTACAAAGGACGAACTTGG | | |
| | ATTACATCAACTTTGGACCCATTGGTTTTGTCGCTGTCGTCAACTGACAGTGATTCACCACTGGTGAT | | |
| | GATAAAAATGATGGAAGAAGAGTTGAAAGTCACTTTTTTCTTTGGCCTGTCCCCATCTTTCTGTGACA | | |
| | TCACAATGGGTCTGATCTGCATTTCACTTCCAGCTGCTGGTAGGTCTTTAGCAGGCCTCTGGCACCTC | | |
| | AGCAGTCGGAGGCACAGAAGCTGCAAAAGGGATCTTCGAAACTGGGCAGAGAAAAAATAAAGTGGAAT | | |
| | ATTAAGTAAAAGTTGGGCACTAATCTGGATTAACATTCGAGGAAATCAGTTGAGCTGATTTAAGTTGT | | |
| | TTTTTGTTTGTTAGCAGGTGTGGATGTGGGGTTATGTGGTCATGCTCAGATCTACCTAAATCACCCCA | | |
| | GAGCTTTATGTCTTTTATTCATTCTAAATCTTATTAACCGGAATATGTAGGACCATTTCAATACCTTG | | |
| | TAATCCTCCAAGCTTCAATCTGCACACACTTTCTATGAGGGCAGGTACAACTATTAAGAGATTTTGAA | | |
| | CATTAAGTTAGTCCACAAATATTCAGTGGGCATCTACTAGGTGACAGCCACTGTGCTATAATTAGAGA | | |
| | CTTTTTACTATAAGCATCAAAAACAGATAAGGCTCTTCCTGGCAGAGTTTACAGCCTGGTGTACTTGC | | |
| | TAATGTCTCTTTAATTAGGTGAAGAATTTTTTTTTTCTATCGAAATTACTAATCAGTTGGGGAAAAAA | | |
| | ATACTATAGCAGACAGCACTAATGTCATCAACAAACATTGTTCTTCTCCGTGTCCTGGGTACAACATC | | |
| | GAATAATATTTCTTGGCCTCCTTTCCGCTTCTCCTCTCTGCTGTTCCTCTCTACAAGAACCTGGGAGG | | |
| | CCAACGCCTAAAGATCATAATATCACAATGGAAGGAACCTAGATTCCTAAATGACTGCATAGGACAGA | | |
| | TCCCATCTCCTCCACCCAATACATTATTAGACTGAACTGTGACCTGAAATGAGCAATAAACTCTGTAT | | |
| | TAATTCACTGAAATGTTGGGGTTGCTTGTTATAGTAGTCGGTCCATCATGACCAGTAAAACATAAATC | | |
| | AAAAGTTAATGTAATTGTTATCCCATTATTTAGAGCGAAATAAATGTTGAATATATGGACTTTCTCAG | | |
| | ATTAGGAAATACCAATTAAAAATATAATAAATAGCT | | |
| | ORF Start: ATG at 47 | | ORF Stop: TGA at 1505 |
| | SEQ ID NO: 20 | 486 aa | MW at 55756.3 kD |
| NOV3b, | MDSSVIQRKKVAVIGGGLVGSLQACFLAKRNFQIDVYEAREDTRVATFTRGRSINLALSHRGRQALKA | | |
| CG127322-01 | | | |
| Protein | VGLEDQIVSQGIPMRARMIHSLSGKKSAIPYGTKSQYILSVSRENLNKDLLTAAEKYPNVKMHFNHRL | | |
| Sequence | | | |
| | LKCNPEEGMITVLGSDKVPKDVTCDLIVGCDGAYSTVRSHLMKKPRFDYSQQYIPHGYMELTIPPKNG | | |
| | DYAMEPNYLHIWPRNTFMMIALPNMNKSFTCTLFMPFEEFEKLLTSNDVVDFFQKYFPDAIPLIGEKL | | |
| | LVQDFFLLPAQPMISVKCSSFHFKSHCVLLGDAAHAIVPFFGQGMNAGFEDCLVFDELMDKFSNDLSL | | |
| | CLPVFSRLRIPDDHAISDLSMYNYIEMRAHVNSSWFIFQKNMERFLHAIMPSTFIPLYTMVTFSRIRY | | |
| | HEAVQRWHWQKKVINKGLFFLGSLIAISSTYLLIHYMSPRSFLCLRRPWNWIAHFRNTTCFPAKAVDS | | |
| | LEQISNLISR | | |
| | SEQ ID NO: 21 | 1380 bp | |
| NOV3c, | CGCGGATCCACCATGGACTCATCTGTCATTCAAAGGAAAAAAGTAGCTGTCATTGGTGGTGGCTTGGT | | |
| CG127322-04 | | | |
| DNA Sequence | TGGCTCATTACAAGCATGCTTTCTTGCAAAGAGGAATTTCCAGATTGATGTATATGAAGCTAGGGAAG | | |
| | ATACTCGAGTGGCTACCTTCACACGTGGAAGAAGCATTAACTTAGCCCTTTCTCATAGAGGACGACAA | | |
| | GCCTTGAAAGCTGTTGGCCTGGAAGATCAGATTGTATCCCAAGGTATTCCCATGAGAGCAAGAATGAT | | |
| | CCACTCTCTTTCAGGAAAAAAGTCTGCAATTCCCTATGGGACAAAGTCTCAGTATATTCTTTCTGTAA | | |
| | GCAGAGAAAATCTAAACAAGGATCTATTGACTGCTGCTGAGAAATACCCCAATGTGAAAATGCACTTT | | |
| | AACCACAGGCTGTTGAAATGTAATCCAGAGGAAGGAATGATCACAGTGCTTGGATCTGACAAAGTTCC | | |
| | CAAAGATGTCACTTGTGACCTCATTGTAGGATGTGATGGAGCCTATTCAACTGTCAGATCTCACCTGA | | |
| | TGAAGAAACCTCGCTTTGATTACAGTCAGCAGTACATTCCTCATGGGTACATGGAGTTGACTATTCCA | | |
| | CCTAAGAACGGAGATAACAAATCATTCACATGTACTTTGTTCATGCCCTTTGAAGAGTTTGAAAAACT | | |
| | TCTAACCAGTAATGATGTGGTAGATTTCTTCCAGAAATACTTCCCGGATGCCATCCCTCTAATTGGAG | | |
| | AGAAACTCCTAGTGCAAGATTTCTTCCTGTTGCCTGCCCAGCCCATGATATCTGTAAAGTGCTCTTCA | | |
| | TTTCACTTTAAATCTCACTGTGTACTGCTGGGAGATGCAGCTCATGCTATAGTGCCGTTTTTTGGGCA | | |
| | AGGAATGAATGCGGGCTTTGAAGACTGCTTGGTATTTGATGAGTTAATGGATAAATTCAGTAACGACC | | |
| | TTAGTTTGTGTCTTCCTGTGTTCTCAAGATTGAGAATCCCAGATGATCACGCGATTTCGGACCTATCC | | |
| | ATGTACAATTACATAGAGAAGAACATGGAGAGATTTCTTCATGCGATTATGCCATCGACCTTTATCCC | | |
| | TCTCTATACAATGGTCACTTTTTCCAGAATAAGATACCATGAGGCTGTGCAGCGTTGGCATTGGCAAA | | |
| | AAAAGGTGATAAACAAAGGACTCTTTTTCTTGGGATCACTGATAGCCATCAGCAGTATCTACCTACTT | | |
| | ATACACTACATGTCACCACGATCTTTCCTCCGCTTGAGAAGACCATGGAACTGGATAGCTCACTTCCG | | |
| | GAATACAACATGTTTCCCCGCAAAGGCCGTGGACTCCCTAGAACAAATTTCCAATCTCATTAGCAGGT | | |
| | GAGCGGCCGCTTTTTTCCTT | | |
| | ORF Start: at 1 | | ORF Stop: TGA at 1360 |
| | SEQ ID NO: 22 | 453 aa | MW at 51681.4 kD |
| NOV3c, | RGSTMDSSVIQRKKVAVIGGGLVGSLQACFLAKRNFQIDVYEAREDTRVATFTRGRSINLALSHRGRQ | | |
| CG127322-04 | | | |
| Protein | ALKAVGLEDQIVSQGIPMRARMIHSLSGKKSAIPYGTKSQYILSVSRENLNKDLLTAAEKYPNVKMHF | | |
| Sequence | | | |
| | NHRLLKCNPEEGMITVLGSDKVPKDVTCDLIVGCDGAYSTVRSHLMKKPRFDYSQQYIPHGYMELTIP | | |
| | PKNGDNKSFTCTLFMPFEEFEKLLTSNDVVDFFQKYFPDAIPLIGEKLLVQDFFLLPAQPMISVKCSS | | |
| | FHFKSHCVLLGDAAHAIVPFFGQGMNAGFEDCLVFDELMDKFSNDLSLCLPVFSRLRIPDDHAISDLS | | |
| | MYNYIEKNMERFLHAIMPSTFIPLYTMVTFSRIRYHEAVQRWHWQKKVINKGLFFLGSLIAISSIYLL | | |
| | IHYMSPRSFLRLRRPWNWIAHFRNTTCFPAKAVDSLEQISNLISR | | |
| | SEQ ID NO: 23 | 1452 bp | |
| NOV3d, | CGCGGATCCACCATGGACTCATCTGTCATTCAAAGGAAAAAAGTAGCTGTCATTGGTGGTGGCTTGGT | | |
| CG127322-03 | | | |
| DNA Sequence | TGGTTCATTACAAGCATGCTTTCTTGCAAAGAGGAATTTCCAGATTGATGTATATGAAGCTAGGGAAG | | |
| | ATACTCGAGTGGCTACCTTCACACGTGGAAGAAGCATTAACTTAGCCCTTTCTCATAGAGGACGACAA | | |
| | GCCTTGAAAGCTGTTGGCCTGGAAGATCAGATTGTATCCCAAGGTATTCCCATGAGAGCAAGAATGAT | | |
| | CCACTCTCTTTCAGGAAAAAAGTCTGCAATTCCCTATGGGACAAAGTCTCAGTATATTCTTTCTGTAA | | |
| | GCAGAGAAAATCTAAACAAGGATCTATTGACTGCTGCTGAGAAATACCCCAATGTGAAGATGCACTTT | | |
| | AACCACAGGCTGTTGAAATGTAATCCAGAGGAAGGAATGATCACAGTGCTTGGATCTGACAAAGTTCC | | |
| | CAAAGATGTCACTTGTGACCTCATTGTAGGATGTGATGGAGCCTATTCAACTGTCAGATCTCACCTGA | | |
| | TGAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGAT | | |
| | GATTGCACTTCCTAACATGAACAAATCATTCACATGTACTTTGTTCATGCCCTTTGAAGAGTTTGAAA | | |
| | AACTTCTAACCAGTAATGATGTGGTAGATTTCTTCCAGAAATACTTTCCGGATGCCATCCCTCTAATT | | |
| | GGAGAGAAACTCCTAGTGCAAGATTTCTTCCTGTTGCCTGCCCAGCCCATGATATCTGTAAAGTGCTC | | |
| | TTCATTTCACTTTAAATCTCACTGTGTACTGCTGGGAGATGCAGCTCATGCTATAGTGCCGTTTTTTG | | |
| | GGCAAGGAATGAATGCGGGCTTTGAAGACTGCTTGGTATTTGATGAGTTAATGGATAAATTCAGTAAC | | |
| | GACCTTAGTTTGTGTCTTCCAGTGTTCTCAAGATTGAGAATCCCAGATGATCACGCGATTTCAGACCT | | |
| | ATCCATGTACAATTACATAGAGAAGAACATGGAGAGATTTCTTCATGCGATTATGCCATCGACCTTTA | | |
| | TCCCTCTCTATACAATGGTCACTTTTTCCAGAATAAGATACCATGAGGCTGTGCAGCGTTGGCATTGG | | |
| | CAAAAAAAGGTGATAAACAAAGGACTCTTTTTCTTGGGATCACTGATAGCCATCAGCAGTACCTACCT | | |
| | ACTTATACACTACATGTCACCACGATCTTTCCTCCGCTTGAGAAGACCATGGAACTGGATAGCTCACT | | |
| | TCCGGAATACAACATGTTTCCCCGCAAAGGCCGTGGACTCCCTAGAACAAATTTCCAATCTCATTAGC | | |
| | AGGTGAGCGGCCGCTTTTTTCCTT | | |
| | ORF Start: at 1 | | ORF Stop: TGA at 1432 |
| | SEQ ID NO: 24 | 477 aa | MW at 54605.9 kD |
| NOV3d, | RGSTMDSSVIQRKKVAVIGGGLVGSLQACFLAKRNFQIDVYEAREDTRVATFTRGRSINLALSHRGRQ | | |
| CG127322-03 | | | |
| Protein | ALKAVGLEDQIVSQGIPMRARMIHSLSGKKSAIPYGTKSQYILSVSRENLNKDLLTAAEKYPNVKMHF | | |
| Sequence | | | |
| | NHRLLKCNPEEGMITVLGSDKVPKDVTCDLIVGCDGAYSTVRSHLMKKPRFDYSQQYIPHGYMELTIP | | |
| | PKNGDYAMEPNYLHIWPRNTFMMIALPNMNKSFTCTLFMPFEEFEKLLTSNDVVDFFQKYFPDAIPLI | | |
| | GEKLLVQDFFLLPAQPMISVKCSSFHFKSHCVLLGDAAHAIVPFFGQGMNAGFEDCLVFDELMDKFSN | | |
| | DLSLCLPVFSRLRIPDDHAISDLSMYNYIEKNMERFLHAIMPSTFIPLYTMVTFSRIRYHEAVQRWHW | | |
| | QKKVINKGLFFLGSLIAISSTYLLIHYMSPRSFLRLRRPWNWIAHFRNTTCFPAKAVDSLEQISNLIS | | |
| | R | | |
| | SEQ ID NO: 25 | 1485 bp | |
| NOV3e, | ACCATGGGCCACCATCACCACCATCACGACTCATCTGTCATTCAAAGGAAAAAAGTAGCTGTCATTGG | | |
| 259357595 | | | |
| DNA Sequence | TGGTGGCTTGGTTGGTTCATTACAAGCATGCTTTCTTGCAAAGAGGAATTTCCAGATTGATGTATATG | | |
| | AAGCTAGGGAAGATACTCGAGTGGCTACCTTCACACGTGGAAGAAGCATTAACTTAGCCCTTTCTCAT | | |
| | AGAGGACGACAAGCCTTGAAAGCTGTTGGCCTGGAAGATCAGATTGTATCCCAAGGTATTCCCATGAG | | |
| | AGCAAGAATGATCCACTCTCTTTCAGGAAAAAAGTCTGCAATTCCCTATGGGACAAAGTCTCAGTATA | | |
| | TTCTTTCTGTAAGCAGAGAAAATCTAAACAAGGATCTATTGACTGCTGCTGAGAAATACCCCAATGTG | | |
| | AAAATGCACTTTAACCACAGGCTGTTGAAATGTAATCCAGAGGAAGGAATGATCACAGTGCTTGGATC | | |
| | TGACAAAGTTCCCAAAGATGTCACTTGTGACCTCATTGTAGGATGTGATGGAGCCTATTCAACTGTCA | | |
| | GATCTCACCTGATGAAGAAACCTCGCTTTGATTACAGTCAGCAGTACATTCCTCATGGGTACATGGAG | | |
| | TTGACTATTCCACCTAAGAACGGAGATTATGCCATGGAACCTAATTATCTGCATATTTGGCCTAGAAA | | |
| | TACCTTTATGATGATTGCACTTCCTAACATGAACAAATCATTCACATGTACTTTGTTCATGCCCTTTG | | |
| | AAGAGTTTGAAAAACTTCTAACCAGTAATGATGTGGTAGATTTCTTCCAGAAATACTTTCCGGATGCC | | |
| | ATCCCTCTAATTGGAGAGAAACTCCTAGTGCAAGATTTCTTCCTGTTGCCTGCCCAGCCCATGATATC | | |
| | TGTAAAGTGCTCTTCATTTCACTTTAAATCTCACTGTGTACTGCTGGGAGATGCAGCTCATGCTATAG | | |
| | TGCCGTTTTTTGGGCAAGGAATGAATGCGGGCTTTGAAGACTGCTTGGTATTTGATGAGTTAATGGAT | | |
| | AAATTCAGTAACGACCTTAGTTTGTGTCTTCCTGTGTTCTCAAGATTGAGAATCCCAGATGATCACGC | | |
| | GATTTCAGACCTATCCATGTACAATTACATAGAGATGCGAGCACATGTCAACTCAAGCTGGTTCATTT | | |
| | TTCAGAAGAACATGGAGAGATTTCTTCATGCGATTATGCCATCGACCTTTATCCCTCTCTATACAATG | | |
| | GTCACTTTTTCCAGAATAAGATACCATGAGGCTGTGCAGCGTTGGCATTGGCAAAAAAAGGTGATAAA | | |
| | CAAAGGACTCTTTTTCTTGGGATCACTGATAGCCATCAGCAGTACCTACCTACTTATACACTACATGT | | |
| | CACCACGATCTTTCCTCCGCTTGAGAAGACCATGGAACTGGATAGCTCACTTCCGGAATACAACATGT | | |
| | TTCCCCGCAAAGGCCGTGGACTCCCTAGAACAAATTTCCAATCTCATTAGCAGGTGA | | |
| | ORF Start: at 1 | | ORF Stop: TGA at 1483 |
| | SEQ ID NO: 26 | 494 aa | MW at 56790.3 kD |
| NOV3e, | TMGHHHHHHDSSVIQRKKVAVIGGGLVGSLQACFLAKRNFQIDVYEAREDTRVATFTRGRSINLALSH | | |
| 259357595 | | | |
| Protein | RGRQALKAVGLEDQIVSQGIPMRARMIHSLSGKKSAIPYGTKSQYILSVSRENLNKDLLTAAEKYPNV | | |
| Sequence | | | |
| | KMHFNHRLLKCNPEEGMITVLGSDKVPKDVTCDLIVGCDGAYSTVRSHLMKKPRFDYSQQYIPHGYME | | |
| | LTIPPKNGDYAMEPNYLHIWPRNTFMMIALPNMNKSFTCTLFMPFEEFEKLLTSNDVVDFFQKYFPDA | | |
| | IPLIGEKLLVQDFFLLPAQPMISVKCSSFHFKSHCVLLGDAAHAIVPFFGQGMNAGFEDCLVFDELMD | | |
| | KFSNDLSLCLPVFSRLRIPDDHAISDLSMYNYIEMRAHVNSSWFIFQKNMERFLHAIMPSTFIPLYTM | | |
| | VTFSRIRYHEAVQRWHWQKKVINKGLFFLGSLIAISSTYLLIHYMSPRSFLRLRRPWNWIAHFRNTTC | | |
| | FPAKAVDSLEQISNLISR | | |
| | SEQ ID NO: 27 | 1491 bp | |
| NOV3f, | CGCGGATCCACCATGGACTCATCTGTCATTCAAAGGAAAAAAGTAGCTGTCATTGGTGGTGGCTTGGT | | |
| 255637561 | | | |
| DNA Sequence | TGGTTCATTACAAGCATGCTTTCTTGCAAAGAGGAATTTCCAGATTGATGTATATGAAGCTAGGGAAG | | |
| | ATACTCGAGTGGCTACCTTCACACGTGGAAGAAGCATTAACTTAGCCCTTTCTCATAGAGGACGACAA | | |
| | GCCTTGAAAGCTGTTGGCCTGGAAGATCAGATTGTATCCCAAGGTATTCCCATGAGAGCAAGAATGAT | | |
| | CCACTCTCTTTCAGGAAAAAAGTCTGCAATTCCCTATGGGACAAAGTCTCAGTATATTCTTTCTGTAA | | |
| | GCAGAGAAAATCTAAACAAGGATCTATTGACTGCTGCTGAGAAATACCCCAATGTGAAAATGCACTTT | | |
| | AACCACAGGCTGTTGAAATGTAATCCAGAGGAAGGAATGATCACAGTGCTTGGATCTGACAAAGTTCC | | |
| | CAAAGATGTCACTTGTGACCTCATTGTAGGATGTGATGGAGCCTATTCAACTGTCAGATCTCACCTGA | | |
| | TGAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGAT | | |
| | GATTGCACTTCCTAACATGAACAAATCATTCACATGTACTTTGTTCATGCCCTTTGAAGAGTTTGAAA | | |
| | AACTTCTAACCAGTAATGATGTGGTAGATTTCTTCCAGAAATACTTTCCGGATGCCATCCCTCTAATT | | |
| | GGAGAGAAACTCCTAGTGCAAGATTTCTTCCTGTTGCCTGCCCAGCCCATGATATCTGTAAAGTGCTC | | |
| | TTCATTTCACTTTAAATCTCACTGTGTACTGCTGGGAGATGCAGCTCATGCTATAGTGCCGTTTTTTG | | |
| | GGCAAGGAATGAATGCGGGCTTTGAAGACTGCTTGGTATTTGATGAGTTAATGGATAAATTCAGTAAC | | |
| | GACCTTAGTTTGTGTCTTCCTGTGTTCTCAAGATTGAGAATCCCAGATGATCACGCGATTTCAGACCT | | |
| | ATCCATGTACAATTACATAGAGATGCGAGCACATGTCAACTCAAGCTGGTTCATTTTTCAGAAGAACA | | |
| | TGGAGAGATTTCTTCATGCGATTATGCCATCGACCTTTATCCCTCTCTATACAATGGTCACTTTTTCC | | |
| | AGAATAAGATACCATGAGGCTGTGCAGCGTTGGCATTGGCAAAAAAAGGTGATAAACAAAGGACTCTT | | |
| | TTTCTTGGGATCACTGATAGCCATCAGCAGTACCTACCTACTTATACACTACATGTCACCACGATCTT | | |
| | TCCTCCGCTTGAGAAGACCATGGAACTGGATAGCTCACTTCCGGAATACAACATGTTTCCCCGCAAAG | | |
| | GCCGTGGACTCCCTAGAACAAATTTCCAATCTCATTAGCAGGTGAGCGGCCGCTTTTTTCCTT | | |
| | ORF Start: at 1 | | ORF Stop: TGA at 1471 |
| | SEQ ID NO: 28 | 490 aa | MW at 56210.7 kD |
| NOV3f, | RGSTMDSSVIQRKKVAVIGGGLVGSLQACFLAKRNFQIDVYEAREDTRVATFTRGRSINLALSHRGRQ | | |
| 255637561 | | | |
| Protein | ALKAVGLEDQIVSQGIPMRARMIHSLSGKKSAIPYGTKSQYILSVSRENLNKDLLTAAEKYPNVKMHF | | |
| Sequence | | | |
| | NHRLLKCNPEEGMITVLGSDKVPKDVTCDLIVGCDGAYSTVRSHLMKKPRFDYSQQYIPHGYMELTIP | | |
| | PKNGDYAMEPNYLHIWPRNTFMMIALPNMNKSFTCTLFMPFEEFEKLLTSNDVVDFFQKYFPDAIPLI | | |
| | GEKLLVQDFFLLPAQPMISVKCSSFHFKSHCVLLGDAAHAIVPFFGQGMNAGFEDCLVFDELMDKFSN | | |
| | DLSLCLPVPSRLRIPDDHAISDLSMYNYIEMRAHVNSSWFIFQKNMERFLHAIMPSTFIPLYTMVTFS | | |
| | RIRYHEAVQRWHWQKKVINKGLFFLGSLIAISSTYLLIHYMSPRSFLRLRRPWNWIAHFRNTTCFPAK | | |
| | AVDSLEQISNLISR | | |
| | SEQ ID NO: 29 | 1482 bp | |
| NOV3g, | ACCATGGACTCATCTGTCATTCAAAGGAAAAAAGTAGCTGTCATTGGTGGTGGCTTGGTTGGTTCAT | | |
| 259357610 | | | |
| DNA Sequence | TACAAGCATGCTTTCTTGCAAAGAGGAATTTCCAGATTGATGTATATGAAGCTAGGGAAGATACTCG | | |
| | AGTGGCTACCTTCACACGTGGAAGAAGCATTAACTTAGCCCTTTCTCATAGAGGACGACAAGCCTTG | | |
| | AAAGCTGTTGGCCTGGAAGATCAGATTGTATCCCAAGGTATTCCCATGAGAGCAAGAATGATCCACT | | |
| | CTCTTTCAGGAAAAAAGTCTGCAATTCCCTATGGGACAAAGTCTCAGTATATTCTTTCTGTAAGCAG | | |
| | AGAAAATCTAAACAAGGATCTATTGACTGCTGCTGAGAAATACCCCAATGTGAAAATGCACTTTAAC | | |
| | CACAGGCTGTTGAAATGTAATCCAGAGGAAGGAATGATCACAGTGCTTGGATCTGACAAAGTTCCCA | | |
| | AAGATGTCACTTGTGACCTCATTGTAGGATGTGATGGAGCCTATTCAACTGTCAGATCTCACCTGAT | | |
| | CAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGA | | |
| | TGATTGCACTTCCTAACATGAACAAATCATTCACATGTACTTTGTTCATGCCCTTTGAAGAGTTTGA | | |
| | AAAACTTCTAACCAGTAATGATGTGGTAGATTTCTTCCAGAAATACTTTCCGGATGCCATCCCTCTA | | |
| | ATTGGAGAGAAACTCCTAGTGCAAGATTTCTTCCTGTTGCCTGCCCAGCCCATGATATCTGTAAAGT | | |
| | GCTCTTCATTTCACTTTAAATCTCACTGTGTACTGCTGGGAGATGCAGCTCATGCTATAGTGCCGTT | | |
| | TTTTGGGCAAGGAATGAATGCGGGCTTTGAAGACTGCTTGGTATTTGATGAGTTAATGGATAAATTC | | |
| | AGTAACGACCTTAGTTTGTGTCTTCCTGTGTTCTCAAGATTGAGAATCCCAGATGATCACGCGATTT | | |
| | CAGACCTATCCATGTACAATTACATAGAGATGCGAGCACATGTCAACTCAAGCTGGTTCATTTTTCA | | |
| | GAAGAACATGGAGAGATTTCTTCATGCGATTATGCCATCGACCTTTATCCCTCTCTATACAATGGTC | | |
| | ACTTTTTCCAGAATAAGATACCATGAGGCTGTGCAGCGTTGGCATTGGCAAAAAAAGGTGATAAACA | | |
| | AAGGACTCTTTTTCTTGGGATCACTGATAGCCATCAGCAGTACCTACCTACTTATACACTACATGTC | | |
| | ACCACGATCTTTCCTCCGCTTGAGAAGACCATGGAACTGGATAGCTCACTTCCGGAATACAACATGT | | |
| | TTCCCCGCAAAGGCCGTGGACTCCCTAGAACAAATTTCCAATCTCATTAGCAGGCACCATCACCACC | | |
| | ATCACTGA | | |
| | ORF Start: at 1 | | ORF Stop: TGA at 1480 |
| | SEQ ID NO: 30 | 493 aa | MW at 56733.3 kD |
| NOV3g, | TMDSSVIQRKKVAVIGGGLVGSLQACFLAKRNFQIDVYEAREDTRVATFTRGRSINLALSHRGRQAL | | |
| 259357610 | | | |
| Protein | KAVGLEDQIVSQGIPMRARMIHSLSGKKSAIPYGTKSQYILSVSRENLNKDLLTAAEKYPNVKMHFN | | |
| Sequence | | | |
| | HRLLKCNPEEGMITVLGSDKVPKDVTCDLIVGCDGAYSTVRSHLMKKPRFDYSQQYIPHGYMELTIP | | |
| | PKNGDYAMEPNYLHIWPRNTFMMIALPNMNKSFTCTLFMPFEEFEKLLTSNDVVDFFQKYFPDAIPL | | |
| | IGEKLLVQDFFLLPAQPMISVKCSSFHFKSHCVLLGDAAHAIVPFFGQGMNAGFEDCLVFDELMDKF | | |
| | SNDLSLCLPVFSRLRIPDDHAISDLSMYNYIEMRAHVNSSWFIFQKNMERFLHAIMPSTFIPLYTMV | | |
| | TFSRIRYHEAVQRWHWQKKVINKGLFFLGSLIAISSTYLLIHYMSPRSFLRLRRPWNWIAHFRNTTC | | |
| | FPAKAVDSLEQISNLISRHHHHHH | | |
| | SEQ ID NO:31 | 1464 bp | |
| NOV3h, | ACCATGGACTCATCTGTCATTCAAAGGAAAAAAGTAGCTGTCATTGGTGGTGGCTTGGTTGGTTCAT | | |
| 259347911 | | | |
| DNA Sequence | TACAAGCATGCTTTCTTGCAAAGAGGAATTTCCAGATTGATGTATATGAAGCTAGGGAAGATACTCG | | |
| | AGTGGCTACCTTCACACGTGGAAGAAGCATTAACTTAGCCCTTTCTCATAGAGGACGACAAGCCTTG | | |
| | AAAGCTGTTGGCCTGGAAGATCAGATTGTATCCCAAGGTATTCCCATGAGAGCAAGAATGATCCACT | | |
| | CTCTTTCAGGAAAAAAGTCTGCAATTCCCTATGGGACAAAGTCTCAGTATATTCTTTCTGTAAGCAG | | |
| | AGAAAATCTAAACAAGGATCTATTGACTGCTGCTGAGAAATACCCCAATGTGAAAATGCACTTTAAC | | |
| | CACAGGCTGTTGAAATGTAATCCAGAGGAAGGAATGATCACAGTGCTTGGATCTGACAAAGTTCCCA | | |
| | AAGATGTCACTTGTGACCTCATTGTAGGATGTGATGGAGCCTATTCAACTGTCAGATCTCACCTGAT | | |
| | GAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGA | | |
| | TGATTGCACTTCCTAACATGAACAAATCATTCACATGTACTTTGTTCATGCCCTTTGAAGAGTTTGA | | |
| | AAAACTTCTAACCAGTAATGATGTGGTAGATTTCTTCCAGAAATACTTTCCGGATGCCATCCCTCTA | | |
| | ATTGGAGAGAAACTCCTAGTGCAAGATTTCTTCCTGTTGCCTGCCCAGCCCATGATATCTGTAAAGT | | |
| | GCTCTTCATTTCACTTTAAATCTCACTGTGTACTGCTGGGAGATGCAGCTCATGCTATAGTGCCGTT | | |
| | TTTTGGGCAAGGAATGAATGCGGGCTTTGAAGACTGCTTGGTATTTGATGAGTTAATGGATAAATTC | | |
| | AGTAACGACCTTAGTTTGTGTCTTCCTGTGTTCTCAAGATTGAGAATCCCAGATGATCACGCGATTT | | |
| | CAGACCTATCCATGTACAATTACATAGAGATGCGAGCACATGTCAACTCAAGCTGGTTCATTTTTCA | | |
| | GAAGAACATGGAGAGATTTCTTCATGCGATTATGCCATCGACCTTTATCCCTCTCTATACAATGGTC | | |
| | ACTTTTTCCAGAATAAGATACCATGAGGCTGTGCAGCGTTGGCATTGGCAAAAAAAGGTGATAAACA | | |
| | AAGGACTCTTTTTCTTGGGATCACTGATAGCCATCAGCAGTACCTACCTACTTATACACTACATGTC | | |
| | ACCACGATCTTTCCTCCGCTTGAGAAGACCATGGAACTGGATAGCTCACTTCCGGAATACAACATGT | | |
| | TTCCCCGCAAAGGCCGTGGACTCCCTAGAACAAATTTCCAATCTCATTAGCAGGTGA | | |
| | ORF Start: at 1 | | ORF Stop: TGA at 1462 |
| | SEQ ID NO: 32 | 487 aa | MW at 55910.4 kD |
| NOV3h, | TMDSSVIQRKKVAVIGGGLVGSLQACFLAKRNFQIDVYEAREDTRVATFTRGRSINLALSHRGRQAL | | |
| 259347911 | | | |
| Protein | KAVGLEDQIVSQGIPMRARMIHSLSGKKSAIPYGTKSQYILSVSRENLNKDLLTAAEKYPNVKMHFN | | |
| Sequence | | | |
| | HRLLKCNPEEGMITVLGSDKVPKDVTCDLIVGCDGAYSTVRSHLMKKPRFDYSQQYIPHGYMELTIP | | |
| | PKNGDYAMEPNYLHIWPRNTFMMIALPNMNKSFTCTLFMPFEEFEKLLTSNDVVDFFQKYFPDAIPL | | |
| | IGEKLLVQDFFLLPAQPMISVKCSSFHFKSHCVLLGDAAHAIVPFFGQGMNAGFHDCLVFDELMDKF | | |
| | SNDLSLCLPVFSRLRIPDDHAISDLSMYNYIEMRAHVNSSWFIFQKNMERFLHAIMPSTFIPLYTMV | | |
| | TFSRIRYHEAVQRWHWQKKVINKGLFFLGSLIAISSTYLLIHYMSPRSFLRLRRPWNWIAHFRNTTC | | |
| | FPAKAVDSLEQISNLISR | | |
| | SEQ ID NO: 33 | 1462 bp | |
| NOV3i, | CATGGACTCATCTGTCATTCAAAGGAAAAAAGTAGCTGTCATTGGTGGTGGCTTGGTTGGTTCATTAC | | |
| 259347915 | | | |
| DNA Sequence | AAGCATGCTTTCTTGCAAAGAGGAATTTCCAGATTGATGTATATGAAGCTAGGGAAGATACTCGAGTG | | |
| | GCTACCTTCACACGTGGAAGAAGCATTAACTTAGCCCTTTCTCATAGAGGACGACAAGCCTTGAAAGC | | |
| | TGTTGGCCTGGAAGATCAGATTGTATCCCAAGGTATTCCCATGAGAGCAAGAATGATCCACTCTCTTT | | |
| | CAGGAAAAAAGTCTGCAATTCCCTATGGGACAAAGTCTCAGTATATTCTTTCTGTAAGCAGAGAAAAT | | |
| | CTAAACAAGGATCTATTGACTGCTGCTGAGAAATACCCCAATGTGAAAATGCACTTTAACCACAGGCT | | |
| | GTTGAAATGTAATCCAGAGGAAGGAATGATCACAGTGCTTGGATCTGACAAAGTTCCCAAAGATGTCA | | |
| | CTTGTGACCTCATTGTAGGATGTGATGGAGCCTATTCAACTGTCAGATCTCACCTGATGAAGAAACCT | | |
| | CGCTTTGATTACAGTCAGCAGTACATTCCTCATGGGTACATGGAGTTGACTATTCCACCTAAGAACGG | | |
| | AGATTATGCCATGGAACCTAATTATCTGCATATTTGGCCTAGAAATACCTTTATGATGATTGCACTTC | | |
| | CTAACATGAACAAATCATTCACATGTACTTTGTTCATGCCCTTTGAAGAGTTTGAAAAACTTCTAACC | | |
| | AGTAATGATGTGGTAGATTTCTTCCAGAAATACTTTCCGGATGCCATCCCTCTAATTGGAGAGAAACT | | |
| | CCTAGTGCAAGATTTCTTCCTGTTGCCTGCCCAGCCCATGATATCTGTAAAGTGCTCTTCATTTCACT | | |
| | TTAAATCTCACTGTGTACTGCTGCGAGATGCAGCTCATGCTATAGTGCCGTTTTTTGGGCAAGGAATG | | |
| | AATGCGGGCTTTGAAGACTGCTTGGTATTTGATGAGTTAATGGATAAATTCAGTAACGACCTTAGTTT | | |
| | GTGTCTTCCTGTGTTCTCAAGATTGAGAATCCCAGATGATCACGCGATTTCAGACCTATCCATGTACA | | |
| | ATTACATAGAGATGCGAGCACATGTCAACTCAAGCTGGTTCATTTTTCAGAAGAACATGGAGAGATTT | | |
| | CTTCATGCGATTATGCCATCGACCTTTATCCCTCTCTATACAATGGTCACTTTTTCCAGAATAAGATA | | |
| | CCATGAGGCTGTGCAGCGTTGGCATTGGCAAAAAAAGGTGATAAACAAAGGACTCTTTTTCTTGGGAT | | |
| | CACTGATAGCCATCAGCAGTACCTACCTACTTATACACTACATGTCACCACGATCTTTCCTCCGCTTG | | |
| | AGAAGACCATGGAACTGGATAGCTCACTTCCGGAATACAACATGTTTCCCCGCAAAGGCCGTGGACTC | | |
| | CCTAGAACAAATTTCCAATCTCATTAGCAGGTGA | | |
| | ORF Start: ATG at 2 | | ORF Stop: TGA at 1460 |
| | SEQ ID NO: 34 | 486 aa | MW at 55809.3 kD |
| NOV3i, | MDSSVIQRKKVAVIGGGLVGSLQACFLAKRNFQIDVYEAREDTRVATFTRGRSINLALSHRGRQALKA | | |
| 259347915 | | | |
| Protein | VGLEDQIVSQGIPMRARMIHSLSGKKSAIPYGTKSQYILSVSRENLNKDLLTAAEKYPNVKMHFNHRL | | |
| Sequence | | | |
| | LKCNPEEGMITVLGSDKVPKDVTCDLIVGCDGAYSTVRSHLMKKPRFDYSQQYIPHGYMELTIPPKNG | | |
| | DYAMEPNYLHIWPRNTFMMIALPNMNKSFTCTLFMPFEEFEKLLTSNDVVDFFQKYFPDAIPLIGEKL | | |
| | LVQDFFLLPAQPMISVKCSSFHFKSHCVLLGDAAHAIVPFFGQGMNAGFEDCLVFDELMDKFSNDLSL | | |
| | CLPVFSRLRIPDDHAISDLSMYNYIEMRAHVNSSWFIFQKNMERFLHAIMPSTFIPLYTMVTFSRIRY | | |
| | HEAVQRWHWQKKVINKGLFFLGSLIAISSTYLLIHYMSPRSFLRLRRPWNWIAHFRNTTCFPAKAVDS | | |
| | LEQISNLISR | | |
| | SEQ ID NO: 35 | 1470 bp | |
| NOV3j, | GGATCCACCATGGACTCATCTGTCATTCAAAGGAAAAAAGTAGCTGTCATTGGTGGTGGCTTGGTTGG | | |
| 260568545 | | | |
| DNA Sequence | TTCATTACAAGCATGCTTTCTTGCAAAGAGGAATTTCCAGATTGATGTATATGAAGCTAGGGAAGATA | | |
| | CTCGAGTGGCTACCTTCACACGTGGAAGAAGCATTAACTTAGCCCTTTCTCATAGAGGACGACAAGCC | | |
| | TTGAAAGCTGTTGGCCTGGAAGATCAGATTGTATCCCAAGGTATTCCCATGAGAGCAAGAATGATCCA | | |
| | CTCTCTTTCAGGAAAAAAGTCTGCAATTCCCTATGGGACAAAGTCTCAGTATATTCTTTCTGTAAGCA | | |
| | GAGAAAATCTAAACAAGGATCTATTGACTGCTGCTGAGAAATACCCCAATGTGAAAATGCACTTTAAC | | |
| | CACAGGCTGTTGAAATGTAATCCAGAGGAAGGAATGATCACAGTGCTTGGATCTGACAAAGTTCCCAA | | |
| | AGATGTCACTTGTGACCTCATTGTAGGATGTGATGGAGCCTATTCAACTGTCAGATCTCACCTGATGA | | |
| | AGAAACCTCGCTTTGATTACAGTCAGCAGTACATTCCTCATGGGTACATGGAGTTGACTATTCCACCT | | |
| | AAGAACGGAGATTATGCCATGGAACCTAATTATCTGCATATTTGGCCTAGAAATACCTTTATGATGAT | | |
| | TGCACTTCCTAACATGAACAAATCATTCACATGTACTTTGTTCATGCCCTTTGAAGAGTTTGAAAAAC | | |
| | TTCTAACCAGTAATGATGTGGTAGATTTCTTCCAGAAATACTTTCCGGATGCCATCCCTCTAATTGGA | | |
| | GAGAAACTCCTAGTGCAAGATTTCTTCCTGTTGCCTGCCCAGCCCATGATATCTGTAAAGTGCTCTTC | | |
| | ATTTCACTTTAAATCTCACTGTGTACTGCTGGGAGATGCAGCTCATGCTATAGTGCCGTTTTTTGGGC | | |
| | AAGGAATGAATGCGGGCTTTGAAGACTGCTTGGTATTTGATGAGTTAATGGATAAATTCAGTAACGAC | | |
| | CTTAGTTTGTGTCTTCCTGTGTTCTCAAGATTGAGAATCCCAGATGATCACGCGATTTCAGACCTATC | | |
| | CATGTACAATTACATAGAGATGCGAGCACATGTCAACTCAAGCTGGTTCATTTTTCAGAAGAACATGG | | |
| | AGAGATTTCTTCATGCGATTATGCCATCGACCTTTATCCCTCTCTATACAATGGTCACTTTTTCCAGA | | |
| | ATAAGATACCATGAGGCTGTGCAGCGTTGGCATTGGCAAAAAAAGGTGATAAACAAAGGACTCTTTTT | | |
| | CTTGGGATCACTGATAGCCATCAGCAGTACCTACCTACTTATACACTACATGTCACCACGATCTTTCC | | |
| | TCCGCTTGAGAAGACCATGGAACTGGATAGCTCACTTCCGGAATACAACATGTTTCCCCGCAAAGGCC | | |
| | GTGGACTCCCTAGAACAAATTTCCAATCTCATTAGCAGGTGA | | |
| | ORF Start: at 1 | | ORF Stop: TGA at 1468 |
| | SEQ ID NO: 36 | 489 aa | MW at 56054.5 kD |
| NOV3j, | GSTMDSSVIQRKKVAVIGGGLVGSLQACFLAKRNFQIDVYEAREDTRVATFTRGRSINLALSHRGRQA | | |
| 260568545 | | | |
| Protein | LKAVGLEDQIVSQGIPMRARMIHSLSGKKSAIPYGTKSQYILSVSRENLNKDLLTAAEKYPNVKMHFN | | |
| Sequence | | | |
| | HRLLKCNPEEGMITVLGSDKVPKDVTCDLIVGCDGAYSTVRSHLMKKPRFDYSQQYIPHGYMELTIPP | | |
| | KNGDYAMEPNYLHIWPRNTFMMIALPNMNKSFTCTLFMPFEEFEKLLTSNDVVDFFQKYFPDAIPLIG | | |
| | EKLLVQDFFLLPAQPMISVKCSSFHFKSHCVLLGDAAHAIVPFFGQGMNAGFEDCLVFDELMDKFSND | | |
| | LSLCLPVFSRLRIPDDHAISDLSMYNYIEMRAHVNSSWFIFQKNMERFLHAIMPSTFIPLYTMVTFSR | | |
| | IRYHEAVQRWHWQKKVINKGLFFLGSLIAISSTYLLIHYMSPRSFLRLRRPWNWIAHFRNTTCFPAKA | | |
| | VDSLEQISNLISR | | |
| | SEQ ID NO: 37 | 1491 bp | |
| NOV3k, | CGCGGATCCACCATGGACTCATCTGTCATTCAAAGGAAAAAAGTAGCTGTCATTGGTGGTGGCTTGG | | |
| 255872826 | | | |
| DNA Sequence | TTGGCTCATTACAAGCATGCTTTCTTGCAAAGAGGAATTTCCAGATTGATGTATATGAAGCTAGGGA | | |
| | AGATACTCGAGTGGCTACCTTCACACGTGGAAGAAGCATTAACTTAGCCCTTTCTCATAGAGGACGA | | |
| | CAAGCCTTGAAAGCTGTTGGCCTGGAAGATCAGATTGTATCCCAAGGTATTCCCATGAGAGCAAGAA | | |
| | TGATCCACTCTCTTTCAGGAAAAAAGTCTGCAATTCCCTATGGGACAAAGTCTCAGTATATTCTTTC | | |
| | TGTAAGCAGAGAAAATCTAAACAAGGATCTATTGACTGCTGCTGAGAAATACCCCAATGTGAAAATG | | |
| | CACTTTAACCACAGGCTGTTGAAATGTAATCCAGAGGAAGGAATGATCACAGTGCTTGGATCTGACA | | |
| | AAGTTCCCAAAGATGTCACTTGTGACCTCATTGTAGGATGTGATGGAGCCTATTCAACTGTCAGATC | | |
| | TCACCTGATGAAGAAACCTCGCTTTGATTACAGTCAGCAGTACATTCCTCATGGGTACATGGAGTTG | | |
| | ACTATTCCACCTAAGAACGGAGATTATGCCATGGAACCTAATTATCTGCATATTTGGCCTAGAAATA | | |
| | CCTTTATGATGATTGCACTTCCTAACATGAACAAATCATTCACATGTACTTTGTTCATGCCCTTTGA | | |
| | AGAGTTTGAAAAACTTCTAACCAGTAATGATGTGGTAGATTTCTTCCAGAAATACTTTCCGGATGCC | | |
| | ATCCCTCTAATTGGAGAGAAACTCCTAGTGCAAGATTTCTTCCTGTTGCCTGCCCAGCCCATGATAT | | |
| | CTGTAAAGTGCTCTTCATTTCACTTTAAATCTCACTGTGTACTGCTGGGAGATGCAGCTCATGCTAT | | |
| | AGTGCCGTTTTTTGGGCAAGGAATGAATGCGGGCTTTGAAGACTGCTTGGTATTTGATGAGTTAATG | | |
| | GATAAATTCAGTAACGACCTTAGTTTGTGTCTTCCTGTGTTCTCAAGATTGAGAATCCCAGATGATC | | |
| | ACGCGATTTCAGACCTATCCATGTACAATTACATAGAGATGCGAGCACATGTCAACTCAAGCTGGTT | | |
| | CATTTTTCAGAAGAACATGGAGAGATTTCTTCATGCGATTATGCCATCGACCTTTATCCCTCTCTAT | | |
| | ACAATGGTCACTTTTTCCAGAATAAGATACCATGAGGCTGTGCAGCGTTGGCATTGGCAAAAAAAGG | | |
| | TGATAAACAAAGGACTCTTTTTCTTGGGATCACTGATAGCCATCAGCAGTACCTACCTACTTATACA | | |
| | CTACATGTCACCACGATCTTTCCTCTGCTTGAGAAGACCATGGAACTGGATAGCTCACTTCCGGAAT | | |
| | ACAACATGTTTCCCCGCAAAGGCCGTGGACTCCCTAGAACAAATTTCCAATCTCATTAGCAGGTGAG | | |
| | CGGCCGCTTTTTTCCTT | | |
| | ORF Start: at 1 | | ORF Stop: TGA at 1471 |
| | SEQ ID NO: 38 | 490 aa | MW at 56157.7 kD |
| NOV3k, | RGSTMDSSVIQRKKVAVIGGGLVGSLQACFLAKRNFQIDVYEAREDTRVATFTRGRSINLALSHRGR | | |
| 255872826 | | | |
| Protein | QALKAVGLEDQIVSQGIPMRARMIHSLSGKKSAIPYGTKSQYILSVSRENLNKDLLTAAEKYPNVKM | | |
| Sequence | | | |
| | HFNHRLLKCNPEEGMITVLGSDKVPKDVTCDLIVGCDGAYSTVRSHLMKKPRFDYSQQYIPHGYMEL | | |
| | TIPPKNGDYAMEPNYLHIWPRNTFMMIALPNMNKSFTCTLFMPFEEFEKLLTSNDVVDFFQKYFPDA | | |
| | IPLIGEKLLVQDFFLLPAQPMISVKCSSFHFKSHCVLLGDAAHAIVPFFGQGMNAGFEDCLVFDELM | | |
| | DKFSNDLSLCLPVFSRLRIPDDHAISDLSMYNYIEMRAHVNSSWFIFQKNMERFLHAIMPSTFIPLY | | |
| | TMVTFSRIRYHEAVQRWHWQKKVINKGLFFLGSLIAISSTYLLIHYMSPRSFLCLRRPWNWIAHFRN | | |
| | TTCFPAKAVDSLEQISNLISR | | |
| | SEQ ID NO: 39 | 1452 bp | |
| NOV3l, | CGCGGATCCACCATGGACTCATCTGTCATTCAAAGGAAAAAAGTAGCTGTCATTGGTGGTGGCTTGGT | | |
| 255872853 | | | |
| DNA Sequence | TGGTTCATTACAAGCATGCTTTCTTGCAAAGAGGAATTTCCAGATTGATGTATATGAAGCTAGGGAAG | | |
| | ATACTCGAGTGGCTACCTTCACACGTGGAAGAAGCATTAACTTAGCCCTTTCTCATAGAGGACGACAA | | |
| | GCCTTGAAAGCTGTTGGCCTGGAAGATCAGATTGTATCCCAAGGTATTCCCATGAGAGCAAGAATGAT | | |
| | CCACTCTCTTTCAGGAAAAAAGTCTGCAATTCCCTATGGGACAAAGTCTCAGTATATTCTTTCTGTAA | | |
| | GCAGAGAAAATCTAAACAAGGATCTATTGACTGCTGCTGAGAAATACCCCAATGTGAAGATGCACTTT | | |
| | AACCACAGGCTGTTGAAATGTAATCCAGAGGAAGGAATGATCACAGTGCTTGGATCTGACAAAGTTCC | | |
| | CAAAGATGTCACTTGTGACCTCATTGTAGGATGTGATGGAGCCTATTCAACTGTCAGATCTCACCTGA | | |
| | TGAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGAT | | |
| | GATTGCACTTCCTAACATGAACAAATCATTCACATGTACTTTGTTCATGCCCTTTGAAGAGTTTGAAA | | |
| | AACTTCTAACCAGTAATGATGTGGTAGATTTCTTCCAGAAATACTTTCCGGATGCCATCCCTCTAATT | | |
| | GGAGAGAAACTCCTAGTGCAAGATTTCTTCCTGTTGCCTGCCCAGCCCATGATATCTGTAAAGTGCTC | | |
| | TTCATTTCACTTTAAATCTCACTGTGTACTGCTGGGAGATGCAGCTCATGCTATAGTGCCGTTTTTTG | | |
| | GGCAAGGAATGAATGCGGGCTTTGAAGACTGCTTGGTATTTGATGAGTTAATGGATAAATTCAGTAAC | | |
| | GACCTTAGTTTGTGTCTTCCAGTGTTCTCAAGATTGAGAATCCCAGATGATCACGCGATTTCAGACCT | | |
| | ATCCATGTACAATTACATAGAGAAGAACATGGAGAGATTTCTTCATGCGATTATGCCATCGACCTTTA | | |
| | TCCCTCTCTATACAATGGTCACTTTTTCCAGAATAAGATACCATGAGGCTGTGCAGCGTTGGCATTGG | | |
| | CAAAAAAAGGTGATAAACAAAGGACTCTTTTTCTTGGGATCACTGATAGCCATCAGCAGTACCTACCT | | |
| | ACTTATACACTACATGTCACCACGATCTTTCCTCCGCTTGAGAAGACCATGGAACTGGATAGCTCACT | | |
| | TCCGGAATACAACATGTTTCCCCGCAAAGGCCGTGGACTCCCTAGAACAAATTTCCAATCTCATTAGC | | |
| | AGGTGAGCGGCCGCTTTTTTCCTT | | |
| | ORF Start: at 1 | | ORF Stop: TGA at 1432 |
| | SEQ ID NO: 40 | 477 aa | MW at 54605.9 kD |
| NOV3l, | RGSTMDSSVIQRKKVAVIGGGLVGSLQACFLAKRNFQIDVYEAREDTRVATFTRGRSINLALSHRGRQ | | |
| 255872853 | | | |
| Protein | ALKAVGLEDQIVSQGIPMRARMIHSLSGKKSAIPYGTKSQYILSVSRENLNKDLLTAAEKYPNVKMHF | | |
| Sequence | | | |
| | NHRLLKCNPEEGMITVLGSDKVPKDVTCDLIVGCDGAYSTVRSHLMKKPRFDYSQQYIPHGYMELTIP | | |
| | PKNGDYAMEPNYLHIWPRNTFMMIALPNMNKSFTCTLFMPFEEFEKLLTSNDVVDFFQKYFPDAIPLI | | |
| | GEKLLVQDFFLLPAQPMISVKCSSFHFKSHCVLLGDAAHAIVPFFGQGMNAGFEDCLVFDELMDKFSN | | |
| | DLSLCLPVFSRLRIPDDHAISDLSMYNYIEKNMERFLHAIMPSTFIPLYTMVTFSRIRYHEAVQRWHW | | |
| | QKKVINKGLFFLGSLIAISSTYLLIHYMSPRSFLRLRRPWNWIAHFRNTTCFPAKAVDSLEQISNLIS | | |
| | R | | |
| | SEQ ID NO: 41 | 1491 bp | |
| NOV3m, | CGCGGATCCACCATGGACTCATCTGTCATTCAAAGGAAAAAAGTAGCTGTCATTGGTGGTGGCTTGG | | |
| CG127322-02 | | | |
| DNA Sequence | TTGGTTCATTACAAGCATGCTTTCTTGCAAAGAGGAATTTCCAGATTGATGTATATGAAGCTAGGGA | | |
| | AGATACTCGAGTGGCTACCTTCACACGTGGAAGAAGCATTAACTTAGCCCTTTCTCATAGAGGACGA | | |
| | CAAGCCTTGAAAGCTGTTGGCCTGGAAGATCAGATTGTATCCCAAGGTATTCCCATGAGAGCAAGAA | | |
| | TGATCCACTCTCTTTCAGGAAAAAAGTCTGCAATTCCCTATGGGACAAAGTCTCAGTATATTCTTTC | | |
| | TGTAAGCAGAGAAAATCTAAACAAGGATCTATTGACTGCTGCTGAGAAATACCCCAATGTGAAAATG | | |
| | CACTTTAACCACAGGCTGTTGAAATGTAATCCAGAGGAAGGAATGATCACAGTGCTTGGATCTGACA | | |
| | AAGTTCCCAAAGATGTCACTTGTGACCTCATTGTAGGATGTGATGGAGCCTATTCAACTGTCAGATC | | |
| | TCACCTGATGAAGAAACCTCGCTTTGATTACAGTCAGCAGTACATTCCTCATGGGTACATGGAGTTG | | |
| | ACTATTCCACCTAAGAACGGAGATTATGCCATGGAACCTAATTATCTGCATATTTGGCCTAGAAATA | | |
| | CCTTTATGATGATTGCACTTCCTAACATGAACAAATCATTCACATGTACTTTGTTCATGCCCTTTGA | | |
| | AGAGTTTGAAAAACTTCTAACCAGTAATGATGTGGTAGATTTCTTCCAGAAATACTTTCCGGATGCC | | |
| | ATCCCTCTAATTGGAGAGAAACTCCTAGTGCAAGATTTCTTCCTGTTGCCTGCCCAGCCCATGATAT | | |
| | CTGTAAAGTGCTCTTCATTTCACTTTAAATCTCACTGTGTACTGCTGGGAGATGCAGCTCATGCTAT | | |
| | AGTGCCGTTTTTTGGGCAAGGAATGAATGCGGGCTTTGAAGACTGCTTGGTATTTGATGAGTTAATG | | |
| | GATAAATTCAGTAACGACCTTAGTTTGTGTCTTCCTGTGTTCTCAAGATTGAGAATCCCAGATGATC | | |
| | ACGCGATTTCAGACCTATCCATGTACAATTACATAGAGATGCGAGCACATGTCAACTCAAGCTGGTT | | |
| | CATTTTTCAGAAGAACATGGAGAGATTTCTTCATGCGATTATGCCATCGACCTTTATCCCTCTCTAT | | |
| | ACAATGGTCACTTTTTCCAGAATAAGATACCATGAGGCTGTGCAGCGTTGGCATTGGCAAAAAAAGG | | |
| | TGATAAACAAAGGACTCTTTTTCTTGGGATCACTGATAGCCATCAGCAGTACCTACCTACTTATACA | | |
| | CTACATGTCACCACGATCTTTCCTCCGCTTGAGAAGACCATGGAACTGGATAGCTCACTTCCGGAAT | | |
| | ACAACATGTTTCCCCGCAAAGGCCGTGGACTCCCTAGAACAAATTTCCAATCTCATTAGCAGGTGAG | | |
| | CGGCCGCTTTTTTCCTT | | |
| | ORF Start: at 1 | | ORF Stop: TGA at 1471 |
| | SEQ ID NO: 42 | 490 aa | MW at 56210.7 kD |
| NOV3m, | RGSTMDSSVIQRKKVAVIGGGLVGSLQACFLAKRNFQIDVYEAREDTRVATFTRGRSINLALSHRGR | | |
| CG127322-02 | | | |
| Protein | QALKAVGLEDQIVSQGIPMRARMIHSLSGKKSAIPYGTKSQYILSVSRENLNKDLLTAAEKYPNVKM | | |
| Sequence | | | |
| | HFNHRLLKCNPEEGMITVLGSDKVPKDVTCDLIVGCDGAYSTVRSHLMKKPRFDYSQQYIPHGYMEL | | |
| | TIPPKNGDYAMEPNYLHIWPRNTFMMIALPNMNKSFTCTLFMPFEEFEKLLTSNDVVDFFQKYFPDA | | |
| | IPLIGEKLLVQDFFLLPAQPMISVKCSSFHFKSHCVLLGDAAHAIVPFFGQGMNAGFEDCLVFDELM | | |
| | DKFSNDLSLCLPVFSRLRIPDDHAISDLSMYNYIEMRAHVNSSWFIFQKNMERFLHAIMPSTFIPLY | | |
| | TMVTFSRIRYHEAVQRWHWQKKVINKGLFFLGSLIAISSTYLLIHYMSPRSFLRLRRPWNWIAHFRN | | |
| | TTCFPAKAVDSLEQISNLISR | | |
| | SEQ ID NO: 43 | 1464 bp | |
| NOV3n, | ACCATGGACTCATCTGTCATTCAAAGGAAAAAAGTAGCTGTCATTGGTGGTGGCTTGGTTGGTTCAT | | |
| CG127322-05 | | | |
| DNA Sequence | TACAAGCATGCTTTCTTGCAAAGAGGAATTTCCAGATTGATGTATATGAAGCTAGGGAAGATACTCG | | |
| | AGTGGCTACCTTCACACGTGGAAGAAGCATTAACTTAGCCCTTTCTCATAGAGGACGACAAGCCTTG | | |
| | AAAGCTGTTGGCCTGGAAGATCAGATTGTATCCCAAGGTATTCCCATGAGAGCAAGAATGATCCACT | | |
| | CTCTTTCAGGAAAAAAGTCTGCAATTCCCTATGGGACAAAGTCTCAGTATATTCTTTCTGTAAGCAG | | |
| | AGAAAATCTAAACAAGGATCTATTGACTGCTGCTGAGAAATACCCCAATGTGAAAATGCACTTTAAC | | |
| | CACAGGCTGTTGAAATGTAATCCAGAGGAAGGAATGATCACAGTGCTTGGATCTGACAAAGTTCCCA | | |
| | AAGATGTCACTTGTGACCTCATTGTAGGATGTGATGGAGCCTATTCAACTGTCAGATCTCACCTGAT | | |
| | GAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGA | | |
| | TGATTGCACTTCCTAACATGAACAAATCATTCACATGTACTTTGTTCATGCCCTTTGAAGAGTTTGA | | |
| | AAAACTTCTAACCAGTAATGATGTGGTAGATTTCTTCCAGAAATACTTTCCGGATGCCATCCCTCTA | | |
| | ATTGGAGAGAAACTCCTAGTGCAAGATTTCTTCCTGTTGCCTGCCCAGCCCATGATATCTGTAAAGT | | |
| | GCTCTTCATTTCACTTTAAATCTCACTGTGTACTGCTGGGAGATGCAGCTCATGCTATAGTGCCGTT | | |
| | TTTTGGGCAAGGAATGAATGCGGGCTTTGAAGACTGCTTGGTATTTGATGAGTTAATGGATAAATTC | | |
| | AGTAACGACCTTAGTTTGTGTCTTCCTGTGTTCTCAAGATTGAGAATCCCAGATGATCACGCGATTT | | |
| | CAGACCTATCCATGTACAATTACATAGAGATGCGAGCACATGTCAACTCAAGCTGGTTCATTTTTCA | | |
| | GAAGAACATGGAGAGATTTCTTCATGCGATTATGCCATCGACCTTTATCCCTCTCTATACAATGGTC | | |
| | ACTTTTTCCAGAATAAGATACCATGAGGCTGTGCAGCGTTGGCATTGGCAAAAAAAGGTGATAAACA | | |
| | AAGGACTCTTTTTCTTGGGATCACTGATAGCCATCAGCAGTACCTACCTACTTATACACTACATGTC | | |
| | ACCACGATCTTTCCTCCGCTTGAGAAGACCATGGAACTGGATAGCTCACTTCCGGAATACAACATGT | | |
| | TTCCCCGCAAAGGCCGTGGACTCCCTAGAACAAATTTCCAATCTCATTAGCAGGTGA | | |
| | ORF Start: at 1 | | ORF Stop: TGA at 1462 |
| | SEQ ID NO: 44 | 487 aa | MW at 55910.4 kD |
| NOV3n, | TMDSSVIQRKKVAVIGGGLVGSLQACFLAKRNFQIDVYEAREDTRVATFTRGRSINLALSHRGRQAL | | |
| CG127322-05 | | | |
| Protein | KAVGLEDQIVSQGIPMRARMIHSLSGKKSAIPYGTKSQYILSVSRENLNKDLLTAAEKYPNVKMHFN | | |
| Sequence | | | |
| | HRLLKCNPEEGMITVLGSDKVPKDVTCDLIVGCDGAYSTVRSHLMKKPRFDYSQQYIPHGYMELTIP | | |
| | PKNGDYAMEPNYLHIWPRNTFMMIALPNMNKSFTCTLFMPFEEFEKLLTSNDVVDFFQKYFPDAIPL | | |
| | IGEKLLVQDFFLLPAQPMISVKCSSFHFKSHCVLLGDAAHAIVPFFGQGMNAGFEDCLVFDELMDKF | | |
| | SNDLSLCLPVFSRLRIPDDHAISDLSMYNYIEMRAHVNSSWFIFQKNMERFLHAIMPSTFIPLYTMV | | |
| | TFSRIRYHEAVQRWHWQKKVINKGLFFLGSLIAISSTYLLIHYMSPRSFLRLRRPWNWIAHFRNTTC | | |
| | FPAKAVDSLEQISNLISR | | |
| | SEQ ID NO: 45 | 1482 bp | |
| NOV3o, | ACCATGGACTCATCTGTCATTCAAAGGAAAAAAGTAGCTGTCATTGGTGGTGGCTTGGTTGGTTCAT | | |
| CG127322-06 | | | |
| DNA Sequence | TACAAGCATGCTTTCTTGCAAAGAGGAATTTCCAGATTGATGTATATGAAGCTAGGGAAGATACTCG | | |
| | AGTGGCTACCTTCACACGTGGAAGAAGCATTAACTTAGCCCTTTCTCATAGAGGACGACAAGCCTTG | | |
| | AAAGCTGTTGGCCTGGAAGATCAGATTGTATCCCAAGGTATTCCCATGAGAGCAAGAATGATCCACT | | |
| | CTCTTTCAGGAAAAAAGTCTGCAATTCCCTATGGGACAAAGTCTCAGTATATTCTTTCTGTAAGCAG | | |
| | AGAAAATCTAAACAAGGATCTATTGACTGCTGCTGAGAAATACCCCAATGTGAAAATGCACTTTAAC | | |
| | CACAGGCTGTTGAAATGTAATCCAGAGGAAGGAATGATCACAGTGCTTGGATCTGACAAAGTTCCCA | | |
| | AAGATGTCACTTGTGACCTCATTGTAGGATGTGATGGAGCCTATTCAACTGTCAGATCTCACCTGAT | | |
| | GAAGAAACCTCGCTTTGATTACAGTCAGCAGTACATTCCTCATGGGTACATGGAGTTGACTATTCCA | | |
| | CCTAAGAACGGAGATTATGCCATGGAACCTAATTATCTGCATATTTGGCCTAGAAATACCTTTATGA | | |
| | TGATTGCACTTCCTAACATGAACAAATCATTCACATGTACTTTGTTCATGCCCTTTGAAGAGTTTGA | | |
| | AAAACTTCTAACCAGTAATGATGTGGTAGATTTCTTCCAGAAATACTTTCCGGATGCCATCCCTCTA | | |
| | ATTGGAGAGAAACTCCTAGTGCAAGATTTCTTCCTGTTGCCTGCCCAGCCCATGATATCTGTAAAGT | | |
| | GCTCTTCATTTCACTTTAAATCTCACTGTGTACTGCTGGGAGATGCAGCTCATGCTATAGTGCCGTT | | |
| | TTTTGGGCAAGGAATGAATGCGGGCTTTGAAGACTGCTTGGTATTTGATGAGTTAATGGATAAATTC | | |
| | AGTAACGACCTTAGTTTGTGTCTTCCTGTGTTCTCAAGATTGAGAATCCCAGATGATCACGCGATTT | | |
| | CAGACCTATCCATGTACAATTACATAGAGATGCGAGCACATGTCAACTCAAGCTGGTTCATTTTTCA | | |
| | GAAGAACATGGAGAGATTTCTTCATGCGATTATGCCATCGACCTTTATCCCTCTCTATACAATGGTC | | |
| | ACTTTTTCCAGAATAAGATACCATGAGGCTGTGCAGCGTTGGCATTGGCAAAAAAAGGTGATAAACA | | |
| | AAGGACTCTTTTTCTTGGGATCACTGATAGCCATCAGCAGTACCTACCTACTTATACACTACATGTC | | |
| | ACCACGATCTTTCCTCCGCTTGAGAAGACCATGGAACTGGATAGCTCACTTCCGGAATACAACATGT | | |
| | TTCCCCGCAAAGGCCGTGGACTCCCTAGAACAAATTTCCAATCTCATTAGCAGGCACCATCACCACC | | |
| | ATCACTGA | | |
| | ORF Start: at 1 | | ORF Stop: TGA at 1480 |
| | SEQ ID NO: 46 | 493 aa | MW at 56733.3 kD |

| | |
|---|---|
| NOV3o, | TMDSSVIQRKKVAVIGGGLVGSLQACFLAKRNFQIDVYEAREDTRVATFTRGRSINLALSHRGRQAL |
| CG127322-06 | |
| Protein | KAVGLEDQIVSQGIPMRARMIHSLSGKKSAIPYGTKSQYILSVSRENLNKDLLTAAEKYPNVKMHFN |
| Sequence | |
| | HRLLKCNPEEGMITVLGSDKVPKDVTCDLIVGCDGAYSTVRSHLMKKPRFDYSQQYIPHGYMELTIP |
| | PKNGDYAMEPNYLHIWPRNTFMMIALPNMNKSFTCTLFMPFEEFEKLLTSNDVVDFFQKYFPDAIPL |
| | IGEKLLVQDFFLLPAQPMISVKCSSFHFKSHCVLLGDAAHAIVPFFGQGMNAGFEDCLVFDELMDKF |
| | SNDLSLCLPVFSRLRIPDDHAISDLSMYNYIEMRAHVNSSWFIFQKNMERFLHAIMPSTFIPLYTMV |
| | TFSRIRYHEAVQRWHWQKKVINKGLFFLGSLIAISSTYLLIHYMSPRSFLRLRRPWNWIAHFRNTTC |
| | FPAKAVDSLEQISNLISRHHHHHH |

Sequence comparison of the above protein sequences yields the following sequence relationships shown in Table 3B.

TABLE 3B
Comparison of NOV3a against NOV3b through NOV3o.

| | | Identities/ |
|---|---|---|
| | NOV3a | Similarities |
| | Residues/ | for the |
| Protein | Match | Matched |
| Sequence | Residues | Region |
| NOV3b | 10 . . . 494 | 484/485 (99%) |
| | 2 . . . 486 | 484/485 (99%) |
| NOV3c | 10 . . . 494 | 447/485 (92%) |
| | 6 . . . 453 | 447/485 (92%) |
| NOV3d | 10 . . . 494 | 472/485 (97%) |
| | 6 . . . 477 | 472/485 (97%) |
| NOV3e | 1 . . . 494 | 494/494 (100%) |
| | 1 . . . 494 | 494/494 (100%) |
| NOV3f | 10 . . . 494 | 485/485 (100%) |
| | 6 . . . 490 | 485/485 (100%) |
| NOV3g | 10 . . . 494 | 485/485 (100%) |
| | 3 . . . 487 | 485/485 (100%) |
| NOV3h | 10 . . . 494 | 485/485 (100%) |
| | 3 . . . 487 | 485/485 (100%) |
| NOV3i | 10 . . . 494 | 485/485 (100%) |
| | 2 . . . 486 | 485/485 (100%) |
| NOV3j | 10 . . . 494 | 485/485 (100%) |
| | 5 . . . 489 | 485/485 (100%) |
| NOV3k | 10 . . . 494 | 484/485 (99%) |
| | 6 . . . 490 | 484/485 (99%) |
| NOV3l | 10 . . . 494 | 472/485 (97%) |
| | 6 . . . 477 | 472/485 (97%) |
| NOV3m | 10 . . . 494 | 485/485 (100%) |
| | 6 . . . 490 | 485/485 (100%) |
| NOV3n | 10 . . . 494 | 485/485 (100%) |
| | 3 . . . 487 | 485/485 (100%) |
| NOV3o | 10 . . . 494 | 485/485 (100%) |
| | 3 . . . 487 | 485/485 (100%) |

Further analysis of the NOV3a protein yielded the following properties shown in Table 3C.

TABLE 3C
Protein Sequence Properties NOV3a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos.chg 0; neg.chg 1 | | | |
| | H-region: length 5; peak value 0.00 | | | |
| | PSG score: −4.40 | | | |
| | GvH: von Heijne's method for signal seq. recognition | | | |
| | GvH score (threshold: −2.1): −2.97 | | | |
| | possible cleavage site: between 32 and 33 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 2 | | | |
| | INTEGRAL Likelihood = −2.44 Transmembrane | | | |
| | 19-35 | | | |
| | INTEGRAL Likelihood = −4.35 Transmembrane | | | |
| | 434-450 | | | |
| | PERIPHERAL Likelihood = 1.96 (at 272) | | | |
| | ALOM score: −4.35 (number of TMSs: 2) | | | |
| | MTOP: Prediction of membrane topology (Hartmann et al.) | | | |
| | Center position for calculation: 26 | | | |
| | Charge difference: −6.0 C(−1.0) − N(5.0) | | | |
| | N >= C: N-terminal side will be inside | | | |
| | >>> membrane topology: type 3a | | | |
| | MITDISC: discrimination of mitochondrial targeting seq | | | |
| | R content: | 2 Hyd | Moment(75): | 2.28 |
| | Hyd Moment(95): | 3.41 | G content: | 5 |
| | D/E content: | 2 | S/T content: | 4 |

Score: −7.24
Gavel: prediction of cleavage sites for mitochondrial
preseq
R-2 motif at 48 KRN|FQ
NUCDISC: discrimination of nuclear localization signals
pat4: KKPR (4) at 187
pat 7: none
bipartite: none
content of basic residues: 11.3%
NLS Score: −0.22
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 89
COIL: Lupas's algorithm to detect coiled-coil regions
total: 0 residues
--------------------------
Final Results (k = 9/23):
39.1%: endoplasmic reticulum
34.8%: mitochondrial
17.4%: nuclear
4.3%: vesicles of secretory system
4.3%: cytoplasmic
>> prediction for CG127322-07 is end (k = 23)

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABJ05589 | Breast cancer-associated | 10 . . . 494 | 484/485 (99%) | 0.0 |
| | protein 54 - Unidentified, 486 | 2 . . . 486 | 484/485 (99%) | |
| | aa. [WO200259377-A2, | | | |
| | 01 AUG 2002] | | | |
| AAW48252 | Human | 10 . . . 494 | 484/485 (99%) | 0.0 |
| | kynurenine-3-hydroxylase - | 2 . . . 486 | 484/485 (99%) | |
| | Homo sapiens, 486 aa. | | | |
| | [WO9802553-A1, | | | |
| | 22 JAN 1998] | | | |
| AAW48251 | Human | 10 . . . 494 | 484/485 (99%) | 0.0 |
| | kynurenine-3-hydroxylase - | 2 . . . 486 | 484/485 (99%) | |
| | Homo sapiens, 486 aa. | | | |
| | [WO9802553-A1, | | | |
| | 22 JAN 1998] | | | |
| AAW48250 | Rat | 11 . . . 465 | 371/455 (81%) | 0.0 |
| | kynurenine-3-hydroxylase - | 3 . . . 457 | 413/455 (90%) | |
| | Rattus sp, 478 aa. | | | |
| | [W09802553-A1, | | | |
| | 22 JAN 1998] | | | |
| ABB58248 | Drosophila melanogaster | 15 . . . 376 | 187/363 (51%) | e−103 |
| | polypeptide SEQ ID NO | 86 . . . 447 | 247/363 (67%) | |
| | 1536 - Drosophila | | | |
| | melanogaster, 506 aa. | | | |
| | [W0200171042-A2, | | | |
| | 27 SEP 2001] | | | |

In a BLAST search of public sequence datbases, the NOV3a protein was found to have homology to the proteins shown in the BLASTP data in Table 3E.

TABLE 3E
Public BLASTP Results for NOV3a

| | | | Identities/ | |
|---|---|---|---|---|
| Protein | | | Similarities for | |
| Accession | | NOV3a Residues/ | the Matched | Expect |
| Number | Protein/Organism/Length | Match Residues | Portion | Value |
| O15229 | Kynurenine 3-monooxygenase | 10 . . . 494 | 484/485 (99%) | 0.0 |
| | (EC 1.14.13.9) - Homo | 2 . . . 486 | 484/485 (99%) | |
| | sapiens (Human), 486 aa. | | | |
| Q9BS61 | Similar to kynurenine | 10 . . . 428 | 405/419 (96%) | 0.0 |
| | 3-monooxygenase | 2 . . . 407 | 406/419 (96%) | |
| | (Kynurenine 3-hydroxylase) - | | | |
| | Homo sapiens (Human), 407 | | | |
| | aa. | | | |
| Q9MZS9 | L-kynurenine | 10 . . . 473 | 381/464 (82%) | 0.0 |
| | 3-monooxygenase Fpk - Sus | 9 . . . 472 | 420/464 (90%) | |
| | scrofa (Pig), 478 aa. | | | |
| Q91WN4 | Similar to kynurenine | 11 . . . 464 | 375/454 (82%) | 0.0 |
| | 3-hydroxylase - Mus musculus | 3 . . . 456 | 415/454 (90%) | |
| | (Mouse), 479 aa. | | | |
| O88867 | Kynurenine 3-hydroxylase - | 11 . . . 465 | 371/455 (81%) | 0.0 |
| | Rattus norvegicus (Rat), 478 | 3 . . . 457 | 413/455 (90%) | |
| | aa. | | | |

PFam analysis predicts that the NOV3a protein contains the domains shown in the Table 3F.

TABLE 3F
Domain Analysis of NOV3a

| | | Identities/ | |
|---|---|---|---|
| | | Similarities | |
| | NOV3a | for the | |
| | Match | Matched | Expect |
| Pfam Domain | Region | Region | Value |
| FAD_binding_3 | 19 . . . 158 | 27/147 (18%) | 0.034 |
| | | 83/147 (56%) | |
| Monooxygenase | 167 . . . 369 | 55/224 (25%) | 2.8e−52 |
| | | 169/224 (75%) | |

### Example 4

The NOV4 clone was analyzed, and the nucleotide and encoded polypeptide sequences are shown in Table 4A.

TABLE 4A
NOV4 Sequence Analysis

| | SEQ ID NO: 47 | 1690 bp | |
|---|---|---|---|
| NOV4a, | CACCATGCAAAGTTGTGAATCCAGTGGTGACAGTGCGGATGACCCTCTCAGTCGCGGCCTACGGAGAA | | |
| CG140122-07 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCGGGGTGAGGGCGACCACAATCACGACACTGGGGAGGGTG | | |
| | GCCAGGGTGGAGAGGAGCCCCGGGGGGGCAGGTGGGATGAGGATGAGCAGTGGTCGGTGGTGGTGGAG | | |
| | TGCGAGGACTGTGAGCTGATCCCGGCGGACCATGTGATTGTGACCGTGTCGCTAGGTGTGCTAAAGAG | | |
| | GCAGTACACCAGTTTCTTCCGGCCAGGCCTGCCCACAGAGAAGGTGGCTGCCATCCACCGCCTGGGCA | | |
| | TTGGCACCACCGACAAGATCTTTCTGGAATTCGAGGAGCCCTTCTGGGGCCCTGAGTGCAACAGCCTA | | |
| | CAGTTTGTGTGGGAGGACGAAGCAGAGAGCCACACCCTCACCTACCCACCTGAGCTCTGGTACCGCAA | | |
| | GATCTGCGGCTTTGATGTCCTCTACCCGCCTGAGCGCTACGGCCATGTGCTGAGCGGCTGGATCTGCG | | |
| | GGGAGGAGGCCCTCGTCATGGAGAAGTGTGATGACGAGGCAGTGGCCGAGATCTGCACGGAGATGCTG | | |
| | CGTCAGTTCACAGGGAACCCCAACATTCCAAAACCTCGGCGAATCTTGCGCTCGGCCTGGGGCAGCAA | | |
| | CCCTTACTTCCGCGGCTCCTATTCATACACGCAGGTGGGCTCCAGCGGGGCGGATGTGGAGAAGCTGG | | |
| | CCAAGCCCCTGCCGTACACGGAGAGCTCAAAGACAGCGCCCATGCAGGTGCTGTTTTCCGGTGAGGCC | | |
| | ACCCACCGCAAGTACTATTCCACCACCCACGGTGCTCTGCTGTCCGGCCAGCGTGAGGCTGCCCGCCT | | |
| | CATTGAGATGTACCGAGACCTCTTCCAGCAGGGGACCCATCATCACCACCATCACTGA | | |
| | ORF Start: at 2 | | ORF Stop: TGA at 1688 |
| | SEQ ID NO: 48 | 562 aa | MW at 62742.6 kD |
| NOV4a, | TMQSCESSGDSADDPLSRGLRRRGQPRVVVIGAGLAGLAAAKALLEQGFTDVTVLEASSHIGGRVQSV | | |
| CG140122-07 | | | |
| Protein | KLGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPRGEGDHNHDTGEGGQGGEEPRGGRWDEDEQWSVVVECEDCELIPADHVIVTVSLGVLKR | | |
| | QYTSFFRPGLPTEKVAAIHRLGIGTTDKIFLEFEEPFWGPECNSLQFVWEDEAESHTLTYPPELWYRK | | |
| | ICGFDVLYPPERYGHVLSGWICGEEALVMEKCDDEAVAEICTEMLRQFTGNPNIPKPRRILRSAWGSN | | |
| | PYFRGSYSYTQVGSSGADVEKLAKPLPYTESSKTAPMQVLFSGEATHRKYYSTTHGALLSGQREAARL | | |
| | IEMYRDLFQQGTHHHHHH | | |
| | SEQ ID NO: 49 | 1894 bp | |
| NOV4b, | CGCCGCTCGCCGCAGACTTACTTCCCCGGCTCAGCAGGGAAAGGTTCCTAGAAGGTGAGCGCGGACGG | | |
| CG140122-01 | | | |
| DNA Sequence | TATGCAAAGTTGTGAATCCAGTGGTGACAGTGCGGATGACCCTCTCAGTCGCGGCCTACGGAGAAGGG | | |
| | GACAGCCTCGTGTGGTGGTGATCGGCGCCGGCTTGGCTGGCCTGGCTGCAGCCAAAGCACTTCTTGAG | | |
| | CAGGGTTTCACGGATGTCACTGTGCTTGAGGCTTCCAGCCACATCGGAGGCCGTGTGCAGAGTGTGAA | | |
| | ACTTGGACACGCCACCTTTGAGCTGGGAGCCACCTGGATCCATGGCTCCCATGGGAACCCTATCTATC | | |
| | ATCTAGCAGAAGCCAACGGCCTCCTGGAAGAGACAACCGATGGGGAACGCAGCGTGGGCCGCATCAGC | | |
| | CTCTATTCCAAGAATGGCGTGGCCTGCTACCTTACCAACCACGGCCGCAGGATCCCCAAGGACGTGGT | | |
| | TGAGGAATTCAGCGATTTATACAACGAGGTCTATAACTTGACCCAGGAGTTCTTCCGGCACGATAAAC | | |
| | CAGTCAATGCTGAAAGTCAAAATAGCGTGGGGGTGTTCACCCGAGAGGAGGTGCGTAACCGCATCAGG | | |
| | AATGACCCTGACGACCCAGAGGCTACCAAGCGCCTGAAGCTCGCCATGATCCAGCAGTACCTGAAGGT | | |
| | GGAGAGCTGTGAGAGCAGCTCACACAGCATGGACGAGGTGTCCCTGAGCGCCTTCGGGGAGTGGACCG | | |
| | AGATCCCCGGCGCTCACCACATCATCCCCTCGGGCTTCATGCGGGTTGTGGAGCTGCTGGCGGAGGGC | | |
| | ATCCCTGCCCACGTCATCCAGCTAGGGAAACCTGTCCGCTGCATTCACTGGGACCAGGCCTCAGCCCG | | |
| | CCCCAGAGGCCCTGAGATTGAGCCCCGGGGTGAGGGCGACCACAATCACGACACTGGGGAGGGTGGCC | | |
| | AGGGTGGAGAGGAGCCCCGGGGGGGCAGGTGGGATGAGGATGAGCAGTGGTCGGTGGTGGTGGAGTGC | | |
| | GAGGACCGTGAGCTGATCCCGGCGGACCATGTGATTGTGACCGTGTCGCTAGGTGTGCTAAAGAGGCA | | |
| | GTACACCAGTTTCTTCCGGCCAGGCCTGCCCACAGAGAAGGTGGCTGCCATCCACCGCCTGGGCATTG | | |
| | GCACCACCGACAAGATCTTTCTGGAATTCGAGGAGCCCTTCTGGGGCCCTGAGTGCAACAGCCTACAG | | |
| | TTTGTGTGGGAGGACGAAGCGGAGAGCCACACCCTCACCTACCCACCTGAGCTCTGGTACCGCAAGAT | | |
| | CTGCGGCTTTGATGTCCTCTACCCGCCTGAGCGCTACGGCCATGTGCTGAGCGGCTGGATCTGCGGGG | | |
| | AGGAGGCCCTCGTCATGGAGAAGTGTGATGACGAGGCAGTGGCCGAGATCTGCACGGAGATGCTGCGT | | |
| | CAGTTCACAGGGAACCCCAACATTCCAAAACCTCGGCGAATCTTGCGCTCGGCCTGGGGCAGCAACCC | | |
| | TTACTTCCGTGGCTCCTATTCATACACGCAGGTGGGCTCCAGCGGGGCGGATGTGGAGAAGCTGGCCA | | |
| | AGCCCCTGCCGTACACGGAGAGCTCAAAGACAGCGCCCATGCAGGTGCTGTTTTCCGGTGAGGCCACC | | |
| | CACCGCAAGTACTATTCCACCACCCACGGTGCTCTGCTGTCCGGCCAGCGTGAGGCTGCCCGCCTCAT | | |
| | TGAGATGTACCGAGACCTCTTCCAGCAGGGGACCTGAGGGCTGTCCTCGCTGCTGAGAAGAGCCACTA | | |
| | ACTCGTGACCTCCAGCCTGCCCCTTGCTGCCGTGTGCTCCTGCCTTCCTGATCCTCTGTAGAAAGGAT | | |
| | TTTTATCTTCTGTAGAGCTAGCCGCCCTGACTGCCTTCAGACCTGGCCCTGTAGCTTT | | |
| | ORF Start: ATG at 70 | | ORF Stop: TGA at 1735 |
| | SEQ ID NO: 50 | 555 aa | MW at 61871.7 kD |
| NOV4b, | MQSCESSGDSADDPLSRGLRRRGQPRVVVIGAGLAGLAAAKALLEQGFTDVTVLEASSHIGGRVQSVK | | |
| CG140122-01 | | | |
| Protein | LGHATFELGATWIHGSHGNPIYHLAEANGLLEETTDGERSVGRISLYSKNGVACYLTNHGRRIPKDVV | | |
| Sequence | | | |
| | EEFSDLYNEVYNLTQEFFRHDKPVNAESQNSVGVFTREEVRNRIRNDPDDPEATKRLKLAMIQQYLKV | | |
| | ESCESSSHSMDEVSLSAFGEWTEIPGAHHIIPSGFMRVVELLAEGIPAHVIQLGKPVRCIHWDQASAR | | |
| | PRGPEIEPRGEGDHNHDTGEGGQGGEEPRGGRWDEDEQWSVVVECEDRELIPADHVIVTVSLGVLKRQ | | |
| | YTSFFRPGLPTEKVAAIHRLGIGTTDKIFLEFEEPFWGPECNSLQFVWEDEAESHTLTYPPELWYRKI | | |
| | CGFDVLYPPERYGHVLSGWICGEEALVMEKCDDEAVAEICTEMLRQFTGNPNIPKPRRILRSAWGSNP | | |
| | YFRGSYSYTQVGSSGADVEKLAKPLPYTESSKTAPMQVLFSGEATHRKYYSTTHGALLSGQREAARLI | | |
| | EMYRDLFQQGT | | |
| | SEQ ID NO: 51 | 1012 bp | |
| NOV4c, | CACCATGCAAAGTTGTGAATCCAGTGGTGACAGTGCGGATGACCCTCTCAGTCGCGGCCTACGGAGAA | | |
| CG140122-03 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCTGCCGTACACAGAGAGCTCAAAGACAGCGCCCATGCAGG | | |
| | TGCTGTTTTCCGGTGAGGCCACCCACCGCAAGTACTATTCCACCACCCACGGTGCTCTGCTGTCCGGC | | |
| | CAGCGTGAGGCTGCCCGCCTCATTGAGATGTACCGAGACCTCTTCCAGCAGGGGACCTGA | | |
| | ORF Start: at 2 | | ORF Stop: TGA at 1010 |
| | SEQ ID NO: 52 | 336 aa | MW at 37093.2 kD |
| NOV4c, | TMQSCESSGDSADDPLSRGLRRRGQPRVVVIGAGLAGLAAAKALLEQGFTDVTVLEASSHIGGRVQSV | | |
| CG140122-03 | | | |
| Protein | KLGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPLPYTESSKTAPMQVLFSGEATHRKYYSTTHGALLSGQREAARLIEMYRDLFQQGT | | |
| | SEQ ID NO: 53 | 1603 bp | |
| NOV4d, | CACCATGCAAAGTTGTGAATCCAGTGGTGACAGTGCGGATGACCCTCTCAGTCGCGGCCTACGGAGAA | | |
| CG140122-04 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCGGGGTGTGCTAAAGAGGCAGTACACCAGTTTCTTCCGGC | | |
| | CAGGCCTGCCCACAGAGAAGGTGGCTGCCATCCACCGCCTGGGCATTGGCACCACCGACAAGATCTTT | | |
| | CTGGAATTCGAGGAGCCCTTCTGGGGCCCTGAGTGCAACAGCCTACAGTTTGTGTGGGAGGACGAAGC | | |
| | GGAGAGCCACACCCTCACCTACCCACCTGAGCTCTGGTACCGCAAGATCTGCGGCTTTGATGTCCTCT | | |
| | ACCCGCCTGAGCGCTACGGCCATGTGCTGAGCGGCTGGATCTGCGGGGAGGAGGCCCTCGTCATGGAG | | |
| | AAGTGTGATGACGAGGCAGTGGCCGAGATCTGCACGGAGATGCTGCGTCAGTTCACAGGGAACCCCAA | | |
| | CATTCCAAAACCTCGGCGAATCTTGCGCTCGGCCTGGGGCAGCAACCCTTACTTCCGCGGCTCCTATT | | |
| | CATACACGCAGGTGGGCTCCAGCGGGGCGGATGTGGAGAAGCTGGCCAAGCCCCTGCCGTACACGGAG | | |
| | AGCTCAAAGACAGCGCATGGAAGCTCCACAAAGCAGCAGCCTGGTCACCTTTTCTCTTCCAAGTGCCC | | |
| | AGAACAGCCCCTGGATGCTAACAGGGGCGCCGTAAAGCCCATGCAGGTGCTGTTTTCCGGTGAGGCCA | | |
| | CCCACCGCAAGTACTATTCCACCACCCACGGTGCTCTGCTGTCCGGCCAGCGTGAGGCTGCCCGCCTC | | |
| | ATTGAGATGTACCGAGACCTCTTCCAGCAGGGGACCTGA | | |
| | ORF Start: at 2 | | ORF Stop: TGA at 1601 |
| | SEQ ID NO: 54 | 533 aa | MW at 59379.2 kD |
| NOV4d, | TMQSCESSGDSADDPLSRGLRRRGQPRVVVIGAGLAGLAAAKALLEQGFTDVTVLEASSHIGGRVQSV | | |
| CG144122-04 | | | |
| Protein | KLGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPRGVLKRQYTSFFRPGLPTEKVAAIHRLGIGTTDKIFLEFEEPFWGPECNSLQFVWEDEA | | |
| | ESHTLTYPPELWYRKICGFDVLYPPERYGHVLSGWICGEEALVMEKCDDEAVAEICTEMLRQFTGNPN | | |
| | IPKPRRILRSAWGSNPYFRGSYSYTQVGSSGADVEKLAKPLPYTESSKTAHGSSTKQQPGHLFSSKCP | | |
| | EQPLDANRGAVKPMQVLFSGEATHRKYYSTTHGALLSGQREAARLIEMYRDLFQQGT | | |
| | SEQ ID NO: 55 | 1012 bp | |
| NOV4e, | CACCATGCAAAGTTGTGAATCCAGTGGTGACAGTGCGGATGACCCTCTCAGTCGCGGCCTACGGAGAA | | |
| 246864043 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCTGCCGTACACAGAGAGCTCAAAGACAGCGCCCATGCAGG | | |
| | TGCTGTTTTCCGGTGAGGCCACCCACCGCAAGTACTATTCCACCACCCACGGTGCTCTGCTGTCCGGC | | |
| | CAGCGTGAGGCTGCCCGCCTCATTGAGATGTACCGAGACCTCTTCCAGCAGGGGACCTGA | | |
| | ORF Start: at 2 | | ORF Stop: TGA at 1010 |
| | SEQ ID NO: 56 | 336 aa | MW at 37093.2 kD |
| NOV4e, | TMQSCESSGDSADDPLSRGLRRRGQPRVVVIGAGLAGLAAAKALLEQGFTDVTVLEASSHIGGRVQSV | | |
| 246864043 | | | |
| Protein | KLGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPLPYTESSKTAPMQVLFSGEATHRKYYSTTHGALLSGQREAARLIEMYRDLFQQGT | | |
| | SEQ ID NO: 57 | 1603 bp | |
| NOV4f, | CACCATGCAAAGTTGTGAATCCAGTGGTGACAGTGCGGATGACCCTCTCAGTCGCGGCCTACGGAGAA | | |
| 246864086 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCGGGGTGTGCTAAAGAGGCAGTACACCAGTTTCTTCCGGC | | |
| | CAGGCCTGCCCACAGAGAAGGTGGCTGCCATCCACCGCCTGGGCATTGGCACCACCGACAAGATCTTT | | |
| | CTGGAATTCGAGGAGCCCTTCTGGGGCCCTGAGTGCAACAGCCTACAGTTTGTGTGGGAGGACGAAGC | | |
| | GGAGAGCCACACCCTCACCTACCCACCTGAGCTCTGGTACCGCAAGATCTGCGGCTTTGATGTCCTCT | | |
| | ACCCGCCTGAGCGCTACGGCCATGTGCTGAGCGGCTGGATCTGCGGGGAGGAGGCCCTCGTCATGGAG | | |
| | AAGTGTGATGACGAGGCAGTGGCCGAGATCTGCACGGAGATGCTGCGTCAGTTCACAGGGAACCCCAA | | |
| | CATTCCAAAACCTCGGCGAATCTTGCGCTCGGCCTGGGGCAGCAACCCTTACTTCCGCGGCTCCTATT | | |
| | CATACACGCAGGTGGGCTCCAGCGGGGCGGATGTGGAGAAGCTGGCCAAGCCCCTGCCGTACACGGAG | | |
| | AGCTCAAAGACAGCGCATGGAAGCTCCACAAAGCAGCAGCCTGGTCACCTTTTCTCTTCCAAGTGCCC | | |
| | AGAACAGCCCCTGGATGCTAACAGGGGCGCCGTAAAGCCCATGCACGTGCTGTTTTCCGGTGAGGCCA | | |
| | CCCACCGCAAGTACTATTCCACCACCCACGGTGCTCTGCTGTCCGGCCAGCGTGAGGCTGCCCGCCTC | | |
| | ATTGAGATGTACCGAGACCTCTTCCAGCAGGGGACCTGA | | |
| | ORF Start: at 2 | | ORF Stop: TGA at 1601 |
| | SEQ ID NO: 58 | 533 aa | MW at 59379.2 kD |
| NOV4f, | TMQSCESSGDSADDPLSRGLRRRGQPRVVVIGAGLAGLAAAKALLEQGFTDVTVLEASSHIGGRVQSV | | |
| 246864086 | | | |
| Protein | KLGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPRGVLKRQYTSFFRPGLPTEKVAAIHRLGIGTTDKIFLEFEEPFWGPECNSLQFVWEDEA | | |
| | ESHTLTYPPELWYRKICGFDVLYPPERYGHVLSGWICGEEALVMEKCDDEAVAEICTEMLRQFTGNPN | | |
| | IPKPRRILRSAWGSNPYFRGSYSYTQVGSSGADVEKLAKPLPYTESSKTAHGSSTKQQPGHLFSSKCP | | |
| | EQPLDANRGAVKPMQVLFSGEATHRKYYSTTHGALLSGQREAARLIEMYRDLFQQGT | | |
| | SEQ ID NO: 59 | 1693 bp | |
| NOV4g, | CACCATGGGACATCATCACCACCATCACCAAAGTTGTGAATCCAGTGGTGACAGTGCGGATGACCCT | | |
| 258280083 | | | |
| DNA Sequence | CTCAGTCGCGGCCTACGGAGAAGGGGACAGCCTCGTGTGGTGGTGATCGGCGCCGGCTTGGCTGGCC | | |
| | TGGCTGCAGCCAAAGCACTTCTTGAGCAGGGTTTCACGGATGTCACTGTGCTTGAGGCTTCCAGCCA | | |
| | CATCGGAGGCCGTGTGCAGAGTGTGAAACTTGGACACGCCACCTTTGAGCTGGGAGCCACCTGGATC | | |
| | CATGGCTCCCATGGGAACCCTATCTATCATCTAGCAGAAGCCAACGGCCTCCTGGAAGAGACAACCG | | |
| | ATGGGGAACGCAGCGTGGGCCGCATCAGCCTCTATTCCAAGAATGGCGTGGCCTGCTACCTTACCAA | | |
| | CCACGGCCGCAGGATCCCCAAGGACGTGGTTGAGGAATTCAGCGATTTATACAACGAGGTCTATAAC | | |
| | TTGACCCAGGAGTTCTTCCGGCACGATAAACCAGTCAATGCTGAAAGTCAAAATAGCGTGGGGGTGT | | |
| | TCACCCGAGAGGAGGTGCGTAACCGCATCAGGAATGACCCTGACGACCCAGAGGCTACCAAGCGCCT | | |
| | GAAGCTCGCCATGATCCAGCAGTACCTGAAGGTGGAGAGCTGTGAGAGCAGCTCACACAGCATGGAC | | |
| | GAGGTGTCCCTGAGCGCCTTCGGGGAGTGGACCGAGATCCCCGGCGCTCACCACATCATCCCCTCGG | | |
| | GCTTCATGCGGGTTGTGGAGCTGCTGGCGGAGGGCATCCCTGCCCACGTCATCCAGCTAGGGAAACC | | |
| | TGTCCGCTGCATTCACTGGGACCAGGCCTCAGCCCGCCCCAGAGGCCCTGAGATTGAGCCCCGGGGT | | |
| | GAGGGCGACCACAATCACGACACTGGGGAGGGTGGCCAGGGTGGAGAGGAGCCCCGGGGGGGCAGGT | | |
| | GGGATGAGGATGAGCAGTGGTCGGTGGTGGTGGAGTGCGAGGACTGTGAGCTGATCCCGGCGGACCA | | |
| | TGTGATTGTGACCGTGTCGCTAGGTGTGCTAAAGAGGCAGTACACCAGTTTCTTCCGGCCAGGCCTG | | |
| | CCCACAGAGAAGGTGGCTGCCATCCACCGCCTGGGCATTGGCACCACCGACAAGATCTTTCTGGAAT | | |
| | TCGAGGAGCCCTTCTGGGGCCCTGAGTGCAACAGCCTACAGTTTGTGTGGGAGGACGAAGCAGAGAG | | |
| | CCACACCCTCACCTACCCACCTGAGCTCTGGTACCGCAAGATCTGCGGCTTTGATGTCCTCTACCCG | | |
| | CCTGAGCGCTACGGCCATGTGCTGAGCGGCTGGATCTGCGGGGAGGAGGCCCTCGTCATGGAGAAGT | | |
| | GTGATGACGAGGCAGTGGCCGAGATCTGCACGGAGATGCTGCGTCAGTTCACAGGGAACCCCAACAT | | |
| | TCCAAAACCTCGGCGAATCTTGCGCTCGGCCTGGGGCAGCAACCCTTACTTCCGCGGCTCCTATTCA | | |
| | TACACGCAGGTGGGCTCCAGCGGGGCGGATGTGGAGAAGCTGGCCAAGCCCCTGCCGTACACGGAGA | | |
| | GCTCAAAGACAGCGCCCATGCAGGTGCTGTTTTCCGGTGAGGCCACCCACCGCAAGTACTATTCCAC | | |
| | CACCCACGGTGCTGTGCTGTCCGGCCAGCGTGAGGCTGCCCGCCTCATTGAGATGTACCGAGACCTC | | |
| | TTCCAGCAGGGGACCTGA | | |
| | ORF Start: at 2 | | ORF Stop: TGA at 1691 |
| | SEQ ID NO: 60 | 563 aa | MW at 62799.6 kD |
| NOV4g, | TMGHHHHHHQSCESSGDSADDPLSRGLRRRGQPRVVVIGAGLAGLAAAKALLEQGFTDVTVLEASSH | | |
| 258280083 | | | |
| Protein | IGGRVQSVKLGHATFELGATWIHGSHGNPIYHLAEANGLLEETTDGERSVGRISLYSKNGVACYLTN | | |
| Sequence | | | |
| | HGRRIPKDVVEEFSDLYNEVYNLTQEFFRHDKPVNAESQNSVGVFTREEVRNRIRNDPDDPEATKRL | | |
| | KLAMIQQYLKVESCESSSHSMDEVSLSAFGEWTEIPGAHHIIPSGFMRVVELLAEGIPAHVIQLGKP | | |
| | VRCIHWDQASARPRGPEIEPRGEGDHNHDTGEGGQGGEEPRGGRWDEDEQWSVVVECEDCELIPADH | | |
| | VIVTVSLGVLKRQYTSFFRPGLPTEKVAAIHRLGIGTTDKIFLEFEEPFWGPECNSLQFVWEDEAES | | |
| | HTLTYPPELWYRKICGFDVLYPPERYGHVLSGWICGEEALVMEKCDDEAVAEICTEMLRQFTGNPNI | | |
| | PKPRRILRSAWGSNPYFRGSYSYTQVGSSGADVEKLAKPLPYTESSKTAPMQVLFSGEATHRKYYST | | |
| | THGALLSGQREAARLIEMYRDLFQQGT | | |
| | SEQ ID NO: 61 | 1690 bp | |
| NOV4h, | CACCATGCAAAGTTGTGAATCCAGTGGTGACAGTGCGGATGACCCTCTCAGTCGCGGCCTACGGAGA | | |
| 258329988 | | | |
| DNA Sequence | AGGGGACAGCCTCGTGTGGTGGTGATCGGCGCCGGCTTGGCTGGCCTGGCTGCAGCCAAAGCACTTC | | |
| | TTGAGCAGGGTTTCACGGATGTCACTGTGCTTGAGGCTTCCAGCCACATCGGAGGCCGTGTGCAGAG | | |
| | TGTGAAACTTGGACACGCCACCTTTGAGCTGGGAGCCACCTGGATCCATGGCTCCCATGGGAACCCT | | |
| | ATCTATCATCTAGCAGAAGCCAACGGCCTCCTGGAAGAGACAACCGATGGGGAACGCAGCGTGGGCC | | |
| | GCATCAGCCTCTATTCCAAGAATGGCGTGGCCTGCTACCTTACCAACCACGGCCGCAGGATCCCCAA | | |
| | GGACGTGGTTGAGGAATTCAGCGATTTATACAACGAGGTCTATAACTTGACCCAGGAGTTCTTCCGG | | |
| | CACGATAAACCAGTCAATGCTGAAAGTCAAAATAGCGTGGGGGTGTTCACCCGAGAGGAGGTGCGTA | | |
| | ACCGCATCAGGAATGACCCTGACGACCCAGAGGCTACCAAGCGCCTGAAGCTCGCCATGATCCAGCA | | |
| | GTACCTGAAGGTGGAGAGCTGTGAGAGCAGCTCACACAGCATGGACGAGGTGTCCCTGAGCGCCTTC | | |
| | GGGGAGTGGACCGAGATCCCCGGCGCTCACCACATCATCCCCTCGGGCTTCATGCGGGTTGTGGAGC | | |
| | TGCTGGCGGAGGGCATCCCTGCCCACGTCATCCAGCTAGGGAAACCTGTCCGCTGCATTCACTGGGA | | |
| | CCAGGCCTCAGCCCGCCCCAGAGGCCCTGAGATTGAGCCCCGGGGTGAGGGCGACCACAATCACGAC | | |
| | ACTGGGGAGGGTGGCCAGGGTGGAGAGGAGCCCCGGGGGGGCAGGTGGGATGAGGATGAGCAGTGGT | | |
| | CGGTGGTGGTGGAGTGCGAGGACTGTGAGCTGATCCCGGCGGACCATGTGATTGTGACCGTGTCGCT | | |
| | AGGTGTGCTAAAGAGGCAGTACACCAGTTTCTTCCGGCCAGGCCTGCCCACAGAGAAGGTGGCTGCC | | |
| | ATCCACCGCCTGGGCATTGGCACCACCGACAAGATCTTTCTGGAATTCGAGGAGCCCTTCTGGGGCC | | |
| | CTGAGTGCAACAGCCTACAGTTTGTGTGGGAGGACGAAGCAGAGAGCCACACCCTCACCTACCCACC | | |
| | TGAGCTCTGGTACCGCAAGATCTGCGGCTTTGATGTCCTCTACCCGCCTGAGCGCTACGGCCATGTG | | |
| | CTGAGCGGCTGGATCTGCGGGGAGGAGGCCCTCGTCATGGAGAAGTGTGATGACGAGGCAGTGGCCG | | |
| | AGATCTGCACGGAGATGCTGCGTCAGTTCACAGGGAACCCCAACATTCCAAAACCTCGGCGAATCTT | | |
| | GCGCTCGGCCTGGGGCAGCAACCCTTACTTCCGCGGCTCCTATTCATACACGCAGGTGGGCTCCAGC | | |
| | GGGGCGGATGTGGAGAAGCTGGCCAAGCCCCTGCCGTACACGGAGAGCTCAAAGACAGCGCCCATGC | | |
| | AGGTGCTGTTTTCCGGTGAGGCCACCCACCGCAAGTACTATTCCACCACCCACGGTGCTCTGCTGTC | | |
| | CGGCCAGCGTGAGGCTGCCCGCCTCATTGAGATGTACCGAGACCTCTTCCAGCAGGGGACCCATCAT | | |
| | CACCACCATCACTGA | | |
| | ORF Start: at 2 | | ORF Stop: TGA at 1688 |
| | SEQ ID NO: 62 | 562 aa | MW at 62742.6 kD |
| NOV4h, | TMQSCESSGDSADDPLSRGLRRRGQPRVVVIGAGLAGLAAAKALLEQGFTDVTVLEASSHIGGRVQS | | |
| 258329988 | | | |
| Protein | VKLGHATFELGATWIHGSHGNPIYHLAEANGLLEETTDGERSVGRISLYSKNGVACYLTNHGRRIPK | | |
| Sequence | | | |
| | DVVEEFSDLYNEVYNLTQEFFRHDKPVNAESQNSVGVFTREEVRNRIRNDPDDPEATKRLKLAMIQQ | | |
| | YLKVESCESSSHSMDEVSLSAFGEWTEIPGAHHIIPSGFMRVVELLAEGIPAHVIQLGKPVRCIHWD | | |
| | QASARPRGPEIEPRGEGDHNHDTGEGGQGGEEPRGGRWDEDEQWSVVVECEDCELIPADHVIVTVSL | | |
| | GVLKRQYTSFFRPGLPTEKVAAIHRLGIGTTDKIFLEFEEPFWGPECNSLQFVWEDEAESHTLTYPP | | |
| | ELWYRKICGFDVLYPPERYGHVLSGWICGEEALVMEKCDDEAVAEICTEMLRQFTGNPNIPKPRRIL | | |
| | RSAWGSNPYFRGSYSYTQVGSSGADVEKLAKPLPYTESSKTAPMQVLFSGEATHRKYYSTTHGALLS | | |
| | GQREAARLIEMYRDLFQQGTHHHHHH | | |
| | SEQ ID NO: 63 | 1672 bp | |
| NOV4i, | CACCATGCAAAGTTGTGAATCCAGTGGTGACAGTGCGGATGACCCTCTCAGTCGCGGCCTACGGAGAA | | |
| 258280066 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCGGGGTGAGGGCGACCACAATCACGACACTGGGGAGGGTG | | |
| | GCCAGGGTGGAGAGGAGCCCCGGGGGGGCAGGTGGGATGAGGATGAGCAGTGGTCGGTGGTGGTGGAG | | |
| | TGCGAGGACTGTGAGCTGATCCCGGCGGACCATGTGATTGTGACCGTGTCGCTAGGTGTGCTAAAGAG | | |
| | GCAGTACACCAGTTTCTTCCGGCCAGGCCTGCCCACAGAGAAGGTGGCTGCCATCCACCGCCTGGGCA | | |
| | TTGGCACCACCGACAAGATCTTTCTGGAATTCGAGGAGCCCTTCTGGGGCCCTGAGTGCAACAGCCTA | | |
| | CAGTTTGTGTGGGAGGACGAAGCAGAGAGCCACACCCTCACCTACCCACCTGAGCTCTGGTACCGCAA | | |
| | GATCTGCGGCTTTGATGTCCTCTACCCGCCTGAGCGCTACGGCCATGTGCTGAGCGGCTGGATCTGCG | | |
| | GGGAGGAGCCCCTCGTCATGGAGAAGTGTGATGACGAGGCAGTGGCCGAGATCTGCACGGAGATGCTG | | |
| | CGTCAGTTCACAGGGAACCCCAACATTCCAAAACCTCGGCGAATCTTGCGCTCGGCCTGGGGCAGCAA | | |
| | CCCTTACTTCCGCGGCTCCTATTCATACACGCAGGTGGGCTCCAGCGGGGCGGATGTGGAGAAGCTGG | | |
| | CCAAGCCCCTGCCGTACACGGAGAGCTCAAAGACAGCGCCCATGCAGGTGCTGTTTTCCGGTGAGGCC | | |
| | ACCCACCGCAAGTACTATTCCACCACCCACGGTGCTCTGCTGTCCGGCCAGCGTGAGGCTGCCCGCCT | | |
| | CATTGAGATGTACCGAGACCTCTTCCAGCAGGGGACCTGA | | |
| | ORF Start: at 2 | | ORF Stop: TGA at 1670 |
| | SEQ ID NO: 64 | 556 aa | MW at 61919.7 kD |
| NOV4i, | TMQSCESSGDSADDPLSRGLRRRGQPRVVVIGAGLAGLAAAKALLEQGFTDVTVLEASSHIGGRVQSV | | |
| 258280066 | KLGHATFELGATWIHGSHGNPIYHLAEANGLLEETTDGERSVGRISLYSKNGVACYLTNHGRRIPKDV | | |
| Protein | | | |
| Sequence | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPRGEGDHNHDTGEGGQGGEEPRGGRWDEDEQWSVVVECEDCELIPADHVIVTVSLGVLKR | | |
| | QYTSFFRPGLPTEKVAAIHRLGIGTTDKIFLEFEEPFWGPECNSLQFVWEDEAESHTLTYPPELWYRK | | |
| | ICGFDVLYPPERYGHVLSGWICGEEALVMEKCDDEAVAEICTEMLRQFTGNPNIPKPRRILRSAWGSN | | |
| | PYFRGSYSYTQVGSSGADVEKLAKPLPYTESSKTAPMQVLFSGEATHRKYYSTTHGALLSGQREAARL | | |
| | IEMYRDLFQQGT | | |
| | SEQ ID NO: 65 | 1700 bp | |
| NOV4j, | AAGGAAAAAAGCGGCCGCCACCATGCAAAGTTGTGAATCCAGTGGTGACAGTGCGGATGACCCTCTCA | | |
| 254047897 | | | |
| DNA Sequence | GTCGCGGCCTACGGAGAAGGGGACAGCCTCGTGTGGTGGTGATCGGCGCCGGCTTGGCTGGCCTGGCT | | |
| | GCAGCCAAAGCACTTCTTGAGCAGGGTTTCACGGATGTCACTGTGCTTGAGGCTTCCAGCCACATCGG | | |
| | AGGCCGTGTGCAGAGTGTGAAACTTGGACACGCCACCTTTGAGCTGGGAGCCACCTGGATCCATGGCT | | |
| | CCCATGGGAACCCTATCTATCATCTAGCAGAAGCCAACGGCCTCCTGGAAGAGACAACCGATGGGGAA | | |
| | CGCAGCGTGGGCCGCATCAGCCTCTATTCCAAGAATGGCGTGGCCTGCTACCTTACCAACCACGGCCG | | |
| | CAGGATCCCCAAGGACGTGGTTGAGGAATTCAGCGATTTATACAACGAGGTCTATAACTTGACCCAGG | | |
| | AGTTCTTCCGGCACGATAAACCAGTCAATGCTGAAAGTCAAAATAGCGTGGGGGTGTTCACCCGAGAG | | |
| | GAGGTGCGTAACCGCATCAGGAATGACCCTGACGACCCAGAGGCTACCAAGCGCCTGAAGCTCGCCAT | | |
| | GATCCAGCAGTACCTGAAGGTGGAGAGCTGTGAGAGCAGCTCACACAGCATGGACGAGGTGTCCCTGA | | |
| | GCGCCTTCGGGGAGTGGACCGAGATCCCCGGCGCTCACCACATCATCCCCTCGGGCTTCATGCGGGTT | | |
| | GTGGAGCTGCTGGCGGAGGGCATCCCTGCCCACGTCATCCAGCTAGGGAAACCTGTCCGCTGCATTCA | | |
| | CTGGGACCAGGCCTCAGCCCGCCCCAGAGGCCCTGAGATTGAGCCCCGGGGTGAGGGCGACCACAATC | | |
| | ACGACACTGGGGAGGGTGGCCAGGGTGGAGAGGAGCCCCGGGGGGGCAGGTGGGATGAGGATGAGCAG | | |
| | TGGTCGGTGGTGGTGGAGTGCGAGGACTGTGAGCTGATCCCGGCGGACCATGTGATTGTGACCGTGTC | | |
| | GCTAGGTGTGCTAAAGAGGCAGTACACCAGTTTCTTCCGGCCAGGCCTGCCCACAGAGAAGGTGGCTG | | |
| | CCATCCACCGCCTGGGCATTGGCACCACCGACAAGATCTTTCTGGAATTCGAGGAGCCCTTCTGGGGC | | |
| | CCTGAGTGCAACAGCCTACAGTTTGTGTGGGAGGACGAAGCAGAGAGCCACACCCTCACCTACCCACC | | |
| | TGAGCTCTGGTACCGCAAGATCTGCGGCTTTGATGTCCTCTACCCGCCTGAGCGCTACGGCCATGTGC | | |
| | TGAGCGGCTGGATCTGCGGGGAGGAGGCCCTCGTCATGGAGAAGTGTGATGACGAGGCAGTGGCCGAG | | |
| | ATCTGCACGGAGATGCTGCGTCAGTTCACAGGGAACCCCAACATTCCAAAACCTCGGCGAATCTTGCG | | |
| | CTCGGCCTGGGGCAGCAACCCTTACTTCCGCGGCTCCTATTCATACACGCAGGTGGGCTCCAGCGGGG | | |
| | CGGATGTGGAGAAGCTGGCCAAGCCCCTGCCGTACACGGAGAGCTCAAAGACAGCGCCCATGCAGGTG | | |
| | CTGTTTTCCGGTGAGGCCACCCACCGCAAGTACTATTCCACCACCCACGGTGCTCTGCTGTCCGGCCA | | |
| | GCGTGAGGCTGCCCGCCTCATTGAGATGTACCGAGACCTCTTCCAGCAGGGGACCTGATCTAGACTAG | | |
| | ORF Start: at 2 | | ORF Stop: TGA at 1688 |
| | SEQ ID NO: 66 | 562 aa | MW at 62545.5 kD |
| NOV4j, | RKKAAATMQSCESSGDSADDPLSRGLRRRGQPRVVVIGAGLAGLAAAKALLEQGFTDVTVLEASSHIG | | |
| 254047897 | | | |
| Protein | GRVQSVKLGHATFELGATWIHGSHGNPIYHLAEANGLLEETTDGERSVGRISLYSKNGVACYLTNHGR | | |
| Sequence | | | |
| | RIPKDVVEEFSDLYNEVYNLTQEFFRHDKPVNAESQNSVGVFTREEVRNRIRNDPDDPEATKRLKLAM | | |
| | IQQYLKVESCESSSHSMDEVSLSAFGEWTEIPGAHHIIPSGFMRVVELLAEGIPAHVIQLGKPVRCIH | | |
| | WDQASARPRGPEIEPRGEGDHNHDTGEGGQGGEEPRGGRWDEDEQWSVVVECEDCELIPADHVIVTVS | | |
| | LGVLKRQYTSFFRPGLPTEKVAAIHRLGIGTTDKIFLEFEEPFWGPECNSLQFVWEDEAESHTLTYPP | | |
| | ELWYRKICGFDVLYPPERYGHVLSGWICGEEALVMEKCDDEAVAEICTEMLRQFTGNPNIPKPRRILR | | |
| | SAWGSNPYFRGSYSYTQVGSSGADVEKLAKPLPYTESSKTAPMQVLFSGEATHRKYYSTTHGALLSGQ | | |
| | REAARLIEMYRDLFQQGT | | |
| | SEQ ID NO: 67 | 1690 bp | |
| NOV4k, | CACCATGCAAAGTTGTGAATCCAGTGGTGACAGTGCGGATGACCCTCTCAGTCGCGGCCTACGGAGA | | |
| 258329988 | | | |
| DNA Sequence | AGGGGACAGCCTCGTGTGGTGGTGATCGGCGCCGGCTTGGCTGGCCTGGCTGCAGCCAAAGCACTTC | | |
| | TTGAGCAGGGTTTCACGGATGTCACTGTGCTTGAGGCTTCCAGCCACATCGGAGGCCGTGTGCAGAG | | |
| | TGTGAAACTTGGACACGCCACCTTTGAGCTGGGAGCCACCTGGATCCATGGCTCCCATGGGAACCCT | | |
| | ATCTATCATCTAGCAGAAGCCAACGGCCTCCTGGAAGAGACAACCGATGGGGAACGCAGCGTGGGCC | | |
| | GCATCAGCCTCTATTCCAAGAATGGCGTGGCCTGCTACCTTACCAACCACGGCCGCAGGATCCCCAA | | |
| | GGACGTGGTTGAGGAATTCAGCGATTTATACAACGAGGTCTATAACTTGACCCAGGAGTTCTTCCGG | | |
| | CACGATAAACCAGTCAATGCTGAAAGTCAAAATAGCGTGGGGGTGTTCACCCGAGAGGAGGTGCGTA | | |
| | ACCGCATCAGGAATGACCCTGACGACCCAGAGGCTACCAAGCGCCTGAAGCTCGCCATGATCCAGCA | | |
| | GTACCTGAAGGTGGAGAGCTGTGAGAGCAGCTCACACAGCATGGACGAGGTGTCCCTGAGCGCCTTC | | |
| | GGGGAGTGGACCGAGATCCCCGGCGCTCACCACATCATCCCCTCGGGCTTCATGCGGGTTGTGGAGC | | |
| | TGCTGGCGGAGGGCATCCCTGCCCACGTCATCCAGCTAGGGAAACCTGTCCGCTGCATTCACTGGGA | | |
| | CCAGGCCTCAGCCCGCCCCAGAGGCCCTGAGATTGAGCCCCGGGGTGAGGGCGACCACAATCACGAC | | |
| | ACTGGGGAGGGTGGCCAGGGTGGAGAGGAGCCCCGGGGGGGCAGGTGGGATGAGGATGAGCAGTGGT | | |
| | CGGTGGTGGTGGAGTGCGAGGACTGTGAGCTGATCCCGGCGGACCATGTGATTGTGACCGTGTCGCT | | |
| | AGGTGTGCTAAAGAGGCAGTACACCAGTTTCTTCCGGCCAGGCCTGCCCACAGAGAAGGTGGCTGCC | | |
| | ATCCACCGCCTGGGCATTGGCACCACCGACAAGATCTTTCTGGAATTCGAGGAGCCCTTCTGGGGCC | | |
| | CTGAGTGCAACAGCCTACAGTTTGTGTGGGAGGACGAAGCAGAGAGCCACACCCTCACCTACCCACC | | |
| | TGAGCTCTGGTACCGCAAGATCTGCGGCTTTGATGTCCTCTACCCGCCTGAGCGCTACGGCCATGTG | | |
| | CTGAGCGGCTGGATCTGCGGGGAGGAGGCCCTCGTCATGGAGAAGTGTGATGACGAGGCAGTGGCCG | | |
| | AGATCTGCACGGAGATGCTGCGTCAGTTCACAGGGAACCCCAACATTCCAAAACCTCGGCGAATCTT | | |
| | GCGCTCGGCCTGGGGCAGCAACCCTTACTTCCGCGGCTCCTATTCATACACGCAGGTGGGCTCCAGC | | |
| | GGGGCGGATGTGGAGAAGCTGGCCAAGCCCCTGCCGTACACGGAGAGCTCAAAGACAGCGCCCATGC | | |
| | AGGTGCTGTTTTCCGGTGAGGCCACCCACCGCAAGTACTATTCCACCACCCACGGTGCTCTGCTGTC | | |
| | CGGCCAGCGTGAGGCTGCCCGCCTCATTGAGATGTACCGAGACCTCTTCCAGCAGGGGACCCATCAT | | |
| | CACCACCATCACTGA | | |
| | ORF Start: at 2 | | ORF Stop: TGA at 1688 |
| | SEQ ID NO: 68 | 562 aa | MW at 62742.6 kD |
| NOV4k, | TMQSCESSGDSADDPLSRGLRRRGQPRVVVIGAGLAGLAAAKALLEQGFTDVTVLEASSHIGGRVQS | | |
| 258329988 | | | |
| Protein | VKLGHATFELGATWIHGSHGNPIYHLAEANGLLEETTDGERSVGRISLYSKNGVACYLTNHGRRIPK | | |
| Sequence | | | |
| | DVVEEFSDLYNEVYNLTQEFFRDHKPVNAESQNSVGVFTREEVRNRIRNDPDDPEATKRLKLAMIQQ | | |
| | YLKVESCESSSHSMDEVSLSAFGEWTEIPGAHHIIPSGFMRVVELLAEGIPAHVIQLGKPVRCIHWD | | |
| | QASARPRGPEIEPRGEGDHNHDTGEGGQGGEEPRGGRWDEDEQWSVVVECEDCELIPADHVIVTVSL | | |
| | GVLKRQYTSFFRPGLPTEKVAAIHRLGIGTTDKIFLEFEEPFWGPECNSLQFVWEDEAESHTLTYPP | | |
| | ELWYRKICGFDVLYPPERYGHVLSGWICGEEALVMEKCDDEAVAEICTEMLRQFTGNPNIPKPRRIL | | |
| | RSAWGSNPYFRGSYSYTQVGSSGADVEKLAKPLPYTESSKTAPMQVTFSGEATHRKYYSTTHGALLS | | |
| | GQREAARLIEMYRDLFQQGTHHHHHH | | |
| | SEQ ID NO: 69 | 1672 bp | |
| NOV4l, | CACCATGCAAAGTTGTGAATCCAGTGGTGACAGTGCGGATGACCCTCTCAGTCGCGGCCTACGGAGAA | | |
| 258280066 | | | |
| DNA Sequence | GGGGACAGCCTCGTGTGGTGGTGATCGGCGCCGGCTTGGCTGGCCTGGCTGCAGCCAAAGCACTTCTT | | |
| | GAGCAGGGTTTCACGGATGTCACTGTGCTTGAGGCTTCCAGCCACATCGGAGGCCGTGTGCAGAGTGT | | |
| | GAAACTTGGACACGCCACCTTTGAGCTGGGAGCCACCTGGATCCATGGCTCCCATGGGAACCCTATCT | | |
| | ATCATCTAGCAGAAGCCAACGGCCTCCTGGAAGAGACAACCGATGGGGAACGCAGCGTGGGCCGCATC | | |
| | AGCCTCTATTCCAAGAATGGCGTGGCCTGCTACCTTACCAACCACGGCCGCAGGATCCCCAAGGACGT | | |
| | GGTTGAGGAATTCAGCGATTTATACAACGAGGTCTATAACTTGACCCAGGAGTTCTTCCGGCACGATA | | |
| | AACCAGTCAATGCTGAAAGTCAAAATAGCGTGGGGGTGTTCACCCGAGAGGAGGTGCGTAACCGCATC | | |
| | AGGAATGACCCTGACGACCCAGAGGCTACCAAGCGCCTGAAGCTCGCCATGATCCAGCAGTACCTGAA | | |
| | GGTGGAGAGCTGTGAGAGCAGCTCACACAGCATGGACGAGGTGTCCCTGAGCGCCTTCGGGGAGTGGA | | |
| | CCGAGATCCCCGGCGCTCACCACATCATCCCCTCGGGCTTCATGCGGGTTGTGGAGCTGCTGGCGGAG | | |
| | GGCATCCCTGCCCACGTCATCCAGCTAGGGAAACCTGTCCGCTGCATTCACTGGGACCAGGCCTCAGC | | |
| | CCGCCCCAGAGGCCCTGAGATTGAGCCCCGGGGTGAGGGCGACCACAATCACGACACTGGGGAGGGTG | | |
| | GCCAGGGTGGAGAGGAGCCCCGGGGGGGCAGGTGGGATGAGGATGAGCAGTGGTCGGTGGTGGTGGAG | | |
| | TGCGAGGACTGTGAGCTGATCCCGGCGGACCATGTGATTGTGACCGTGTCGCTAGGTGTGCTAAAGAG | | |
| | GCAGTACACCAGTTTCTTCCGGCCAGGCCTGCCCACAGAGAAGGTGGCTGCCATCCACCGCCTGGGCA | | |
| | TTGGCACCACCGACAAGATCTTTCTGGAATTCGAGGAGCCCTTCTGGGGCCCTGAGTGCAACAGCCTA | | |
| | CAGTTTGTGTGGGAGGACGAAGCAGAGAGCCACACCCTCACCTACCCACCTGAGCTCTGGTACCGCAA | | |
| | GATCTGCGGCTTTGATGTCCTCTACCCGCCTGAGCGCTACGGCCATGTGCTGAGCGGCTGGATCTGCG | | |
| | GGGAGGAGGCCCTCGTCATGGAGAAGTGTGATGACGAGGCAGTGGCCGAGATCTGCACGGAGATGCTG | | |
| | CGTCAGTTCACAGGGAACCCCAACATTCCAAAACCTCGGCGAATCTTGCGCTCGGCCTGGGGCAGCAA | | |
| | CCCTTACTTCCGCGGCTCCTATTCATACACGCAGGTGGGCTCCAGCGGGGCGGATGTGGAGAAGCTGG | | |
| | CCAAGCCCCTGCCGTACACGGAGAGCTCAAAGACAGCGCCCATGCAGGTGCTGTTTTCCGGTGAGGCC | | |
| | ACCCACCGCAAGTACTATTCCACCACCCACGGTGCTCTGCTGTCCGGCCAGCGTGAGGCTGCCCGCCT | | |
| | CATTGAGATGTACCGAGACCTCTTCCAGCAGGGGACCTGA | | |
| | ORF Start: at 2 | | ORF Stop: TGA at 1670 |
| | SEQ ID NO: 70 | 556 aa | MW at 61919.7 kD |
| NOV4l, | TMQSCESSGDSADDPLSRGLRRRGQPRVVVIGAGLAGLAAAKALLEQGFTDVTVLEASSHIGGRVQSV | | |
| 258280066 | | | |
| Protein | KLGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYLK | | |
| | VESCESSSHSMDEVSLSAFGEWTEIPGAHHIIPSGFMRVVELLAEGIPAHVIQLGKPVRCIHWDQASA | | |
| | RPRGPEIEPRGEGDHNHDTGEGGQGGEEPRGGRWDEDEQWSVVVECEDCELIPADHVIVTVSLGVLKR | | |
| | QYTSFFRPGLPTEKVAAIHRLGIGTTDKIFLEFEEPFWGPECNSLQFVWEDEAESHTLTYPPELWYRK | | |
| | ICGFDVLYPPERYGHVLSGWICGEEALVMEKCDDEAVAEICTEMLRQFTGNPNIPKPRRILRSAWGSN | | |
| | PYFRGSYSYTQVGSSGADVEKLAKPLPYTESSKTAPMQVLFSGEATHRKYYSTTHGALLSGQREAARL | | |
| | IEMYRDLFQQGT | | |
| | SEQ ID NO: 71 | 1693 bp | |
| NOV4m, | CACCATGGGACATCATCACCACCATCACCAAAGTTGTGAATCCAGTGGTGACAGTGCGGATGACCCT | | |
| 258280083 | | | |
| DNA Sequence | CTCAGTCGCGGCCTACGGAGAAGGGGACAGCCTCGTGTGGTGGTGATCGGCGCCGGCTTGGCTGGCC | | |
| | TGGCTGCAGCCAAAGCACTTCTTGAGCAGGGTTTCACGGATGTCACTGTGCTTGAGGCTTCCAGCCA | | |
| | CATCGGAGGCCGTGTGCAGAGTGTGAAACTTGGACACGCCACCTTTGAGCTGGGAGCCACCTGGATC | | |
| | CATGGCTCCCATGGGAACCCTATCTATCATCTAGCAGAAGCCAACGGCCTCCTGGAAGAGACAACCG | | |
| | ATGGGGAACGCAGCGTGGGCCGCATCAGCCTCTATTCCAAGAATGGCGTGGCCTGCTACCTTACCAA | | |
| | CCACGGCCGCAGGATCCCCAAGGACGTGGTTGAGGAATTCAGCGATTTATACAACGAGGTCTATAAC | | |
| | TTGACCCAGGAGTTCTTCCGGCACGATAAACCAGTCAATGCTGAAAGTCAAAATAGCGTGGGGGTGT | | |
| | TCACCCGAGAGGAGGTGCGTAACCGCATCAGGAATGACCCTGACGACCCAGAGGCTACCAAGCGCCT | | |
| | GAAGCTCGCCATGATCCAGCAGTACCTGAAGGTGGAGAGCTGTGAGAGCAGCTCACACAGCATGGAC | | |
| | GAGGTGTCCCTGAGCGCCTTCGGGGAGTGGACCGAGATCCCCGGCGCTCACCACATCATCCCCTCGG | | |
| | GCTTCATGCGGGTTGTGGAGCTGCTGGCGGAGGGCATCCCTGCCCACGTCATCCAGCTAGGGAAACC | | |
| | TGTCCGCTGCATTCACTGGGACCAGGCCTCAGCCCGCCCCAGAGGCCCTGAGATTGAGCCCCGGGGT | | |
| | GAGGGCGACCACAATCACGACACTGGGGAGGGTGGCCAGGGTGGAGAGGAGCCCCGGGGGGGCAGGT | | |
| | GGGATGAGGATGAGCAGTGGTCGGTGGTGGTGGAGTGCGAGGACTGTGAGCTGATCCCGGCGGACCA | | |
| | TGTGATTGTGACCGTGTCGCTAGGTGTGCTAAAGAGGCAGTACACCAGTTTCTTCCGGCCAGGCCTG | | |
| | CCCACAGAGAAGGTGGCTGCCATCCACCGCCTGGGCATTGGCACCACCGACAAGATCTTTCTGGAAT | | |
| | TCGAGGAGCCCTTCTGGGGCCCTGAGTGCAACAGCCTACAGTTTGTGTGGGAGGACGAAGCAGAGAG | | |
| | CCACACCCTCACCTACCCACCTGAGCTCTGGTACCGCAAGATCTGCGGCTTTGATGTCCTCTACCCG | | |
| | CCTGAGCGCTACGGCCATGTGCTGAGCGGCTGGATCTGCGGGGAGGAGGCCCTCGTCATGGAGAAGT | | |
| | GTGATGACGAGGCAGTGGCCGAGATCTGCACGGAGATGCTGCGTCAGTTCACAGGGAACCCCAACAT | | |
| | TCCAAAACCTCGGCGAATCTTGCGCTCGGCCTGGGGCAGCAACCCTTACTTCCGCGGCTCCTATTCA | | |
| | TACACGCAGGTGGGCTCCAGCGGGGCGGATGTGGAGAAGCTGGCCAAGCCCCTGCCGTACACGGAGA | | |
| | GCTCAAAGACAGCGCCCATGCAGGTGCTGTTTTCCGGTGAGGCCACCCACCGCAAGTACTATTCCAC | | |
| | CACCCACGGTGCTCTGCTGTCCGGCCAGCGTGAGGCTGCCCGCCTCATTGAGATGTACCGAGACCTC | | |
| | TTCCAGCAGGGGACCTGA | | |
| | ORF Start: at 2 | | ORF Stop: TGA at 1691 |
| | SEQ ID NO: 72 | 563 aa | MW at 62799.6 kD |
| NOV4m, | TMGHHHHHHQSCESSGDSADDPLSRGLRRRGQPRVVVIGAGLAGLAAAKALLEQGFTDVTVLEASSH | | |
| 258280083 | | | |
| Protein | IGGRVQSVKLGHATFELGATWIHGSHGNPIYHLAEANGLLEETTDGERSVGRISLYSKNGVACYLTN | | |
| Sequence | | | |
| | HGRRIPKDVVEEFSDLYNEVYNLTQEFFRHDKPVNAESQNSVGVFTREEVRNRIRNDPDDPEATKRL | | |
| | KLAMIQQYLKVESCESSSHSMDEVSLSAFGEWTEIPGAHHIIPSGFMRVVELLAEGIPAHVIQLGKP | | |
| | VRCIHWDQASARPRGPEIEPRGEGDHNHDTGEGGQGGEEPRGGRWDEDEQWSVVVECEDCELIPADH | | |
| | VIVTVSLGVLKRQYTSFFRPGLPTEKVAAIHRLGIGTTDKIFLEFEEPFWGPECNSLQFVWEDEAES | | |
| | HTLTYPPELWYRKICGFDVLYPPERYGHVLSGWICGEEALVMEKCDDEAVAEICTEMLRQFTGNPNI | | |
| | PKPRRILRSAWGSNPYFRGSYSYTQVGSSGADVEKLAKPLPYTESSKTAPMQVLFSGEATHRKYYST | | |
| | THGALLSGQREAARLIEMYRDLFQQGT | | |
| | SEQ ID NO: 73 | 1993 bp | |
| NOV4n, | GGCACGAGGGTCCCGGCGGCGGCTGGAGGAGGAAGCCAGGCGGCTGGCGGAGGAGGAGAGACGGAGG | | |
| CG140122-02 | | | |
| DNA Sequence | AGGCCGAGACCGGAGCGCCGCTCGCCGCAGACTTACTTCCCCGGCTCAGCAGGGAAAGGTTCCTAGA | | |
| | AGGTGAGCGCGGACGGTATGCAAAGTTGTGAATCCAGTGGTGACAGTGCGGATGACCCTCTCAGTCG | | |
| | CGGCCTACGGAGAAGGGGACAGCCTCGTGTGGTGGTGATCGGCGCCGGCTTGGCTGGCCTGGCTGCA | | |
| | GCCAAAGCACTTCTTGAGCAGGGTTTCACGGATGTCACTGTGCTTGAGGCTTCCAGCCACATCGGAG | | |
| | GCCGTGTGCAGAGTGTGAAACTTGGACACGCCACCTTTGAGCTGGGAGCCACCTGGATCCATGGCTC | | |
| | CCATGGGAACCCTATCTATCATCTAGCAGAAGCCAACGGCCTCCTGGAAGAGACAACCGATGGGGAA | | |
| | CGCAGCGTGGGCCGCATCAGCCTCTATTCCAAGAATGGCGTGGCCTGCTACCTTACCAACCACGGCC | | |
| | GCAGGATCCCCAAGGACGTGGTTGAGGAATTCAGCGATTTATACAACGAGGTCTATAACTTGACCCA | | |
| | GGAGTTCTTCCGGCACGATAAACCAGTCAATGCTGAAAGTCAAAATAGCGTGGGGGTGTTCACCCGA | | |
| | GAGGAGGTGCGTAACCGCATCAGGAATGACCCTGACGACCCAGAGGCTACCAAGCGCCTGAAGCTCG | | |
| | CCATGATCCAGCAGTACCTGAAGGTGGAGAGCTGTGAGAGCAGCTCACACAGCATGGACGAGGTGTC | | |
| | CCTGAGCGCCTTCGGGGAGTGGACCGAGATCCCCGGCGCTCACCACATCATCCCCTCGGGCTTCATG | | |
| | CGGGTTGTGGAGCTGCTGGCGGAGGGCATCCCTGCCCACGTCATCCAGCTAGGGAAACCTGTCCGCT | | |
| | GCATTCACTGGGACCAGGCCTCAGCCCGCCCCAGAGGCCCTGAGATTGAGCCCCGGGGTGTGCTAAA | | |
| | GAGGCAGTACACCAGTTTCTTCCGGCCAGGCCTGCCCACAGAGAAGGTGGCTGCCATCCACCGCCTG | | |
| | GGCATTGGCACCACCGACAAGATCTTTCTGGAATTCGAGGAGCCCTTCTGGGGCCCTGAGTGCAACA | | |
| | GCCTACAGTTTGTGTGGGAGGACGAAGCGGAGAGCCACACCCTCACCTACCCACCTGAGCTCTGGTA | | |
| | CCGCAAGATCTGCGGCTTTGATGTCCTCTACCCGCCTGAGCGCTACGGCCATGTGCTGAGCGGCTGG | | |
| | ATCTGCGGGGAGGAGGCCCTCGTCATGGAGAAGTGTGATGACGAGGCAGTGGCCGAGATCTGCACGG | | |
| | AGATGCTGCGTCAGTTCACAGGGAACCCCAACATTCCAAAACCTCGGCGAATCTTGCGCTCGGCCTG | | |
| | GGGCAGCAACCCTTACTTCCGCGGCTCCTATTCATACACGCAGGTGGGCTCCAGCGGGGCGGATGTG | | |
| | GAGAAGCTGGCCAAGCCCCTGCCGTACACGGAGAGCTCAAAGACAGCGCCCATGCAGGTGCTGTTTT | | |
| | CCGGTGAGGCCACCCACCGCAAGTACTATTCCACCACCCACGGTGCTCTGCTGTCCGGCCAGCGTGA | | |
| | GGCTGCCCGCCTCATTGAGATGTACCGAGACCTCTTCCAGCAGGGGACCTGAGGGCTGTCCTCGCTG | | |
| | CTGAGAAGAGCCACTAACTCGTGACCTCCAGCCTGCCCCTTGCTGCCGTGTGCTCCTGCCTTCCTGA | | |
| | TCCTCTGTAGAAAGGATTTTTATCTTCTGTAGAGCTAGCCGCCCTGACTGCCTTCAGACCTGGCCCT | | |
| | GTAGCTTTTCTTTTTCTCCAGGCTGGGCCGTGAGCAGGTGGGCCGTTGAGTTACCTCTGTGCTGGAT | | |
| | CCCGTGCCCCCACTTGCCTACCCTCTGTCCTGCCTTGTTATTGTAAGTGCCTTCAATACTTTGCATT | | |
| | TTGGGATAATAAAAAAGGCTCCCTCCCCTGCAAAAAAAAAAAAAAAAAAA | | |
| | ORF Start: ATG at 152 | | ORF Stop: TGA at 1658 |
| | SEQ ID NO: 74 | 502 aa | MW at 56090.6 kD |
| NOV4n, | MQSCESSGDSADDPLSRGLRRRGQPRVVVIGAGLAGLAAAKALLEQGFTDVTVLEASSHIGGRVQSV | | |
| CG140122-02 | | | |
| Protein | KLGHATFELGATWIHGSHGNPIYHLAEANGLLEETTDGERSVGRISLYSKNGVACYLTNHGRRIPKD | | |
| Sequence | | | |
| | VVEEFSDLYNEVYNLTQEFFRHDKPVNAESQNSVGVFTREEVRNRIRNDPDDPEATKRLKLAMIQQY | | |
| | LKVESCESSSHSMDEVSLSAFGEWTEIPGAHHIIPSGFMRVVELLAEGIPAHVIQLGKPVRCIHWDQ | | |
| | ASARPRGPEIEPRGVLKRQYTSFFRPGLPTEKVAAIHRLGIGTTDKIFLEFEEPFWGPECNSLQFVW | | |
| | EDEAESHTLTYPPELWYRKICGFDVLYPPERYGHVLSGWICGEEALVMEKCDDEAVAEICTEMLRQF | | |
| | TGNPNIPKPRRILRSAWGSNPYFRGSYSYTQVGSSGADVEKLAKPLPYTESSKTAPMQVLFSGEATH | | |
| | RKYYSTTHGALLSGQREAARLIEMYRDLFQQGT | | |
| | SEQ ID NO: 75 | 1513 bp | |
| NOV4o, | CACCATGCAAAGTTGTGAATCCAGTGGTGACAGTGCGGATGACCCTCTCAGTCGCGGCCTACGGAGA | | |
| CG141022-05 | | | |
| DNA Sequence | AGGGGACAGCCTCGTGTGGTGGTGATCGGCGCCGGCTTGGCTGGCCTGGCTGCAGCCAAAGCACTTC | | |
| | TTGAGCAGGGTTTCACGGATGTCACTGTGCTTGAGGCTTCCAGCCACATCGGAGGCCGTGTGCAGAG | | |
| | TGTGAAACTTGGACACGCCACCTTTGAGCTGGGAGCCACCTGGATCCATGGCTCCCATGGGAACCCT | | |
| | ATCTATCATCTAGCAGAAGCCAACGGCCTCCTGGAAGAGACAACCGATGGGGAACGCAGCGTGGGCC | | |
| | GCATCAGCCTCTATTCCAAGAATGGCGTGGCCTGCTACCTTACCAACCACGGCCGCAGGATCCCCAA | | |
| | GGACGTGGTTGAGGAATTCAGCGATTTATACAACGAGGTCTATAACTTGACCCAGGAGTTCTTCCGG | | |
| | CACGATAAACCAGTCAATGCTGAAAGTCAAAATAGCGTGGGGGTGTTCACCCGAGAGGAGGTGCGTA | | |
| | ACCGCATCAGGAATGACCCTGACGACCCAGAGGCTACCAAGCGCCTGAAGCTCGCCATGATCCAGCA | | |
| | GTACCTGAAGGTGGAGAGCTGTGAGAGCAGCTCACACAGCATGGACGAGGTGTCCCTGAGCGCCTTC | | |
| | GGGGAGTGGACCGAGATCCCCGGCGCTCACCACATCATCCCCTCGGGCTTCATGCGGGTTGTGGAGC | | |
| | TGCTGGCGGAGGGCATCCCTGCCCACGTCATCCAGCTAGGGAAACCTGTCCGCTGCATTCACTGGGA | | |
| | CCAGGCCTCAGCCCGCCCCAGAGGCCCTGAGATTGAGCCCCGGGGTGTGCTAAAGAGGCAGTACACC | | |
| | AGTTTCTTCCGGCCAGGCCTGCCCACAGAGAAGGTGGCTGCCATCCACCGCCTGGGCATTGGCACCA | | |
| | CCGACAAGATCTTTCTGGAATTCGAGGAGCCCTTCTGGGGCCCTGAGTGCAACAGCCTACAGTTTGT | | |
| | GTGGGAGGACGAAGCGGAGAGCCACACCCTCACCTACCCACCTGAGCTCTGGTACCGCAAGATCTGC | | |
| | GGCTTTGATGTCCTCTACCCGCCTGAGCGCTACGGCCATGTGCTGAGCGGCTGGATCTGCGGGGAGG | | |
| | AGGCCCTCGTCATGGAGAAGTGTGATGACGAGGCAGTGGCCGAGATCTGCACGGAGATGCTGCGTCA | | |
| | GTTCACAGGGAACCCCAACATTCCAAAACCTCGGCGAATCTTGCGCTCGGCCTGGGGCAGCAACCCT | | |
| | TACTTCCGCGGCTCCTATTCATACACGCAGGTGGGCTCCAGCGGGGCGGATGTGGAGAAGCTGGCCA | | |
| | AGCCCCTGCCGTACACGGAGAGCTCAAAGACAGCGCCCATGCAGGTGCTGTTTTCCGGTGAGGCCAC | | |
| | CCACCGCAAGTACTATTCCACCACCCACGGTGCTCTGCTGTCCGGCCAGCGTGAGGCTGCCCGCCTC | | |
| | ATTGAGATGTACCGAGACCTCTTCCAGCAGGGGACCTGA | | |
| | ORF Start: at 2 | | ORF Stop: TGA at 1511 |
| | SEQ ID NO: 76 | 503 aa | MW at 56191.7 kD |
| NOV4o, | TMQSCESSGDSADDPLSRGLRRRGQPRVVVIGAGLAGLAAAKALLEQGFTDVTVLEASSHIGGRVQS | | |
| CG140122-05 | | | |
| Protein | VKLGHATFELGATWIHGSHGNPIYHLAEANGLLEETTDGERSVGRISLYSKNGVACYLTNHGRRIPK | | |
| Sequence | | | |
| | DVVEEFSDLYNEVYNLTQEFFRHDKPVNAESQNSVGVFTREEVRNRIRNDPDDPEATKRLKLAMIQQ | | |
| | YLKVESCESSSHSMDEVSLSAFGEWTEIPGAHHIIPSGFMRVVELLAEGIPAHVIQLGKPVRCIHWD | | |
| | QASARPRGPEIEPRGVLKRQYTSFFRPGLPTEKVAAIHRLGIGTTDKIFLEFEEPFWGPECNSLQFV | | |
| | WEDEAESHTLTYPPELWYRKICGFDVLYPPERYGHVLSGWICGEEALVMEKCDDEAVAEICTEMLRQ | | |
| | FTGNPNIPKPRRILRSAWGSNPYFRGSYSYTQVGSSGADVEKLAKPLPYTESSKTAPMQVLFSGEAT | | |
| | HRKYYSTTHGALLSGQREAARLIEMYRDLFQQGT | | |
| | SEQ ID NO: 77 | 1693 bp | |
| NOV4p, | CACCATGGGACATCATCACCACCATCACCAAAGTTGTGAATCCAGTGGTGACAGTGCGGATGACCCT | | |
| CG140122-06 | | | |
| DNA Sequence | CTCAGTCGCGGCCTACGGAGAAGGGGACAGCCTCGTGTGGTGGTGATCGGCGCCGGCTTGGCTGGCC | | |
| | TGGCTGCAGCCAAAGCACTTCTTGAGCAGGGTTTCACGGATGTCACTGTGCTTGAGGCTTCCAGCCA | | |
| | CATCGGAGGCCGTGTGCAGAGTGTGAAACTTGGACACGCCACCTTTGAGCTGGGAGCCACCTGGATC | | |
| | CATGGCTCCCATGGGAACCCTATCTATCATCTAGCAGAAGCCAACGGCCTCCTGGAAGAGACAACCG | | |
| | ATGGGGAACGCAGCGTGGGCCGCATCAGCCTCTATTCCAAGAATGGCGTGGCCTGCTACCTTACCAA | | |
| | CCACGGCCGCAGGATCCCCAAGGACGTGGTTGAGGAATTCAGCGATTTATACAACGAGGTCTATAAC | | |
| | TTGACCCAGGAGTTCTTCCGGCACGATAAACCAGTCAATGCTGAAAGTCAAAATAGCGTGGGGGTGT | | |
| | TCACCCGAGAGGAGGTGCGTAACCGCATCAGGAATGACCCTGACGACCCAGAGGCTACCAAGCGCCT | | |
| | GAAGCTCGCCATGATCCAGCAGTACCTGAAGGTGGAGAGCTGTGAGAGCAGCTCACACAGCATGGAC | | |
| | GAGGTGTCCCTGAGCGCCTTCGGGGAGTGCACCGAGATCCCCGGCGCTCACCACATCATCCCCTCGG | | |
| | GCTTCATGCGGGTTGTGGAGCTGCTGGCGGAGGGCATCCCTGCCCACGTCATCCAGCTAGGGAAACC | | |
| | TGTCCGCTGCATTCACTGGGACCAGGCCTCAGCCCGCCCCAGAGGCCCTGAGATTGAGCCCCGGGGT | | |
| | GAGGGCGACCACAATCACGACACTGGGGAGGGTGGCCAGGGTGGAGAGGAGCCCCGGGGGGGCAGGT | | |
| | GGGATGAGGATGAGCAGTGGTCGGTGGTGGTGGAGTGCGAGGACTGTGAGCTGATCCCGGCGGACCA | | |
| | TGTGATTGTGACCGTGTCGCTAGGTGTGCTAAAGAGGCAGTACACCAGTTTCTTCCGGCCAGGCCTG | | |
| | CCCACAGAGAAGGTGGCTGCCATCCACCGCCTGGGCATTGGCACCACCGACAAGATCTTTCTGGAAT | | |
| | TCGAGGAGCCCTTCTGGGGCCCTGAGTGCAACAGCCTACAGTTTGTGTGGGAGGACGAAGCAGAGAG | | |
| | CCACACCCTCACCTACCCACCTGAGCTCTGGTACCGCAAGATCTGCGGCTTTGATGTCCTCTACCCG | | |
| | CCTGAGCGCTACGGCCATGTGCTGAGCGGCTGGATCTGCGGGGAGGAGGCCCTCGTCATGGAGAAGT | | |
| | GTGATGACGAGGCAGTGGCCGAGATCTGCACGGAGATGCTGCGTCAGTTCACAGGGAACCCCAACAT | | |
| | TCCAAAACCTCGGCGAATCTTGCGCTCGGCCTGGGGCAGCAACCCTTACTTCCGCGGCTCCTATTCA | | |
| | TACACGCAGGTGGGCTCCAGCGGGGCGGATGTGGAGAAGCTGGCCAAGCCCCTGCCGTACACGGAGA | | |
| | GCTCAAAGACAGCGCCCATGCAGGTGCTGTTTTCCGGTGAGGCCACCCACCGCAAGTACTATTCCAC | | |
| | CACCCACGGTGCTCTGCTGTCCGGCCAGCGTGAGGCTGCCCGCCTCATTGAGATGTACCGAGACCTC | | |
| | TTCCAGCAGGGGACCTGA | | |
| | ORF Start: at 29 | | ORF Stop: TGA at 1691 |
| | SEQ ID NO: 78 | 554 aa | MW at 61687.4 kD |
| NOV4p, | QSCESSGDSADDPLSRGLRRRGQPRVVVIGAGLAGLAAAKALLEQGFTDVTVLEASSHIGGRVQSVK | | |
| CG140122-06 | | | |
| Protein | LGHATFELGATWIHGSHGNPIYHLAEANGLLEETTDGERSVGRISLYSKNGVACYLTNHGRRIPKDV | | |
| Sequence | | | |
| | VEEFSDLYNEVYNLTQEFFRHDKPVNAESQNSVGVFTREEVRNRIRNDPDDPEATKRLKLAMIQQYL | | |
| | KVESCESSSHSMDEVSLSAFGEWTEIPGAHHIIPSGFMRVVELLAEGIPAHVIQLGKPVRCIHWDQA | | |
| | SARPRGPEIEPRGEGDHNHDTGEGGQGGEEPRGGRWDEDEQWSVVVECEDCELIPADHVIVTVSLGV | | |
| | LKRQYTSFFRPGLPTEKVAAIHRLGIGTTDKIFLEFEEPFWGPECNSLQFVWEDEAESHTLTYPPEL | | |
| | WYRKICGFDVLYPPERYGHVLSGWICGEEALVMEKCDDEAVAEICTEMLRQFTGNPNIPKPRRILRS | | |
| | AWGSNPYFRGSYSYTQVGSSGADVEKLAKPLPYTESSKTAPMQVLFSGEATHRKYYSTTHGALLSGQ | | |
| | REAARLIEMYRDLFQQGT | | |
| | SEQ ID NO: 79 | 1680 bp | |
| NOV4q, | TCCACCATGCAAAGTTGTGAATCCAGTGGTGACAGTGCGGATGACCCTCTCAGTCGCGGCCTACGGA | | |
| CG140122-08 | | | |
| DNA Sequence | GAAGGGGACAGCCTCGTGTGGTGGTGATCGGCGCCGGCTTGGCTGGCCTGGCTGCAGCCAAAGCACT | | |
| | TCTTGAGCAGGGTTTCACGGATGTCACTGTGCTTGAGGCTTCCAGCCACATCGGAGGCCGTGTGCAG | | |
| | AGTGTGAAACTTGGACACGCCACCTTTGAGCTGGGAGCCACCTGGATCCATGGCTCCCATGGGAACC | | |
| | CTATCTATCATCTAGCAGAAGCCAACGGCCTCCTGGAAGAGACAACCGATGGGGAACGCAGCGTGGG | | |
| | CCGCATCAGCCTCTATTCCAAGAATGGCGTGGCCTGCTACCTTACCAACCACGGCCGCAGGATCCCC | | |
| | AAGGACGTGGTTGAGGAATTCAGCGATTTATACAACGAGGTCTATAACTTGACCCAGGAGTTCTTCC | | |
| | GGCACGATAAACCAGTCAATGCTGAAAGTCAAAATAGCGTGGGGGTGTTCACCCGAGAGGAGGTGCG | | |
| | TAACCGCATCAGGAATGACCCTGACGACCCAGAGGCTACCAAGCGCCTGAAGCTCGCCATGATCCAG | | |
| | CAGTACCTGAAGGTGGAGAGCTGTGAGAGCAGCTCACACAGCATGGACGAGGTGTCCCTGAGCGCCT | | |
| | TCGGGGAGTGGACCGAGATCCCCGGCGCTCACCACATCATCCCCTCGGGCTTCATGCGGGTTGTGGA | | |
| | GCTGCTGGCGGAGGGCATCCCTGCCCACGTCATCCAGCTAGGGAAACCTGTCCGCTGCATTCACTGG | | |
| | GACCAGGCCTCAGCCCGCCCCAGAGGCCCTGAGATTGAGCCCCGGGGTGAGGGCGACCACAATCACG | | |
| | ACACTGGGGAGGGTGGCCAGGGTGGAGAGGAGCCCCGGGGGGGCAGGTGGGATGAGGATGAGCAGTG | | |
| | GTCGGTGGTGGTGGAGTGCGAGGACTGTGAGCTGATCCCCGCGGACCATGTGATTGTGACCGTGTCG | | |
| | CTAGGTGTGCTAAAGAGGCAGTACACCAGTTTCTTCCGGCCAGGCCTGCCCACAGAGAAGGTGGCTG | | |
| | CCATCCACCGCCTGGGCATTGGCACCACCGACAAGATCTTTCTGGAATTCGAGCACCCCTTCTGGGG | | |
| | CCCTGAGTGCAACAGCCTACAGTTTGTGTGGGAGGACGAAGCAGAGAGCCACACCCTCACCTACCCA | | |
| | CCTGAGCTCTGGTACCGCAAGATCTGCGGCTTTGATGTCCTCTACCCGCCTGAGCGCTACGGCCATG | | |
| | TGCTGAGCGGCTGGATCTGCGGGGAGGAGGCCCTCGTCATGGAGAAGTGTGATGACGAGGCAGTGGC | | |
| | CGAGATCTGCACGGAGATGCTGCGTCAGTTCACAGGGAACCCCAACATTCCAAAACCTCGGCGAATC | | |
| | TTGCGCTCGGCCTGGGGCAGCAACCCTTACTTCCGCGGCTCCTATTCATACACGCAGGTGGGCTCCA | | |
| | GCGGGGCGGATGTGGAGAAGCTGGCCAAGCCCCTGCCGTACACGGAGAGCTCAAAGACAGCGCCCAT | | |
| | GCAGGTGCTGTTTTCCGGTGAGGCCACCCACCGCAAGTACTATTCCACCACCCACGGTGCTCTGCTG | | |
| | TCCGGCCAGCGTGAGGCTGCCCGCCTCATTGAGATGTACCGAGACCTCTTCCAGCAGGGGACCTGAA | | |
| | AGCTT | | |
| | ORF Start: at 1 | | ORF Stop: TGA at 1672 |
| | SEQ ID NO: 80 | 557 aa | MW at 62006.8 kD |

| | |
|---|---|
| NOV4q, | STMQSCESSGDSADDPLSRGLRRRGQPRVVVIGAGLAGLAAAKALLEQGFTDVTVLEASSHIGGRVQ |
| CG140122-08 | |
| Protein | SVKLGHATFELGATWIHGSHGNPIYHLAEANGLLEETTDGERSVGRISLYSKNGVACYLTNHGRRIP |
| Sequence | |
| | KDVVEEFSDLYNEVYNLTQEFFRHDKPVNAESQNSVGVFTREEVRNRIRNDPDDPEATKRLKLAMIQ |
| | QYLKVESCESSSHSMDEVSLSAFGEWTEIPGAHHIIPSGFMRVVELLAEGIPAHVIQLGKPVRCIHW |
| | DQASARPRGPEIEPRGEGDHNHDTGEGGQGGEEPRGGRWDEDEQWSVVVECEDCELIPADHVIVTVS |
| | LGVLKRQYTSFFRPGLPTEKVAAIHRLGIGTTDKIFLEFEEPFWGPECNSLQFVWEDEAESHTLTYP |
| | PELWYRKICGFDVLYPPERYGHVLSGWICGEEALVMEKCDDEAVAEICTEMLRQFTGNPNIPKPRRI |
| | LRSAWGSNPYFRGSYSYTQVGSSGADVEKLAKPLPYTESSKTAPMQVLFSGEATHRKYYSTTHGALL |
| | SGQREAARLIEMYRDLFQQGT |

Sequence comparison of the above protein sequences yields the following sequence relationships shown in Table 4B.

TABLE 4B
Comparison of NOV4a against NOV4b through NOV4q.

| | | Identities/ |
|---|---|---|
| | NOV4a | Similarities |
| | Residues/ | for the |
| Protein | Match | Matched |
| Sequence | Residues | Region |
| NOV4b | 2 . . . 556 | 554/555 (99%) |
| | 1 . . . 555 | 554/555 (99%) |
| NOV4c | 1 . . . 281 | 281/281 (100%) |
| | 1 . . . 281 | 281/281 (100%) |
| NOV4d | 1 . . . 556 | 503/586 (85%) |
| | 1 . . . 533 | 503/586 (85%) |
| NOV4e | 1 . . . 281 | 281/281 (100%) |
| | 1 . . . 281 | 281/281 (100%) |
| NOV4f | 1 . . . 556 | 503/586 (85%) |
| | 1 . . . 533 | 503/586 (85%) |
| NOV4g | 3 . . . 556 | 554/554 (100%) |
| | 10 . . . 563 | 554/554 (100%) |
| NOV4h | 1 . . . 562 | 562/562 (100%) |
| | 1 . . . 562 | 562/562 (100%) |
| NOV4i | 1 . . . 556 | 556/556 (100%) |
| | 1 . . . 556 | 556/556 (100%) |
| NOV4j | 1 . . . 556 | 556/556 (100%) |
| | 7 . . . 562 | 556/556 (100%) |
| NOV4k | 1 . . . 562 | 562/562 (100%) |
| | 1 . . . 562 | 562/562 (100%) |
| NOV4l | 1 . . . 556 | 556/556 (100%) |
| | 1 . . . 556 | 556/556 (100%) |
| NOV4m | 3 . . . 556 | 554/554 (100%) |
| | 10 . . . 563 | 554/554 (100%) |
| NOV4n | 2 . . . 556 | 502/555 (90%) |
| | 1 . . . 502 | 502/555 (90%) |
| NOV4o | 1 . . . 556 | 503/556 (90%) |
| | 1 . . . 503 | 503/556 (90%) |
| NOV4p | 3 . . . 556 | 554/554 (100%) |
| | 1 . . . 554 | 554/554 (100%) |
| NOV4q | 1 . . . 556 | 556/556 (100%) |
| | 2 . . . 557 | 556/556 (100%) |

Further analysis of the NOV4a protein yielded the following properties shown in Table 4C.

TABLE 4C
Protein Sequence Properties NOV4a

| SignalP | Cleavage site between residues 42 and 43 | | | |
|---|---|---|---|---|
| analysis: | | | | |
| PSORT II | PSG: a new signal peptide prediction method | | | |
| analysis: | N-region: length 10; pos.chg 0; neg.chg 2 | | | |
| | H-region: length 2; peak value 0.00 | | | |
| | PSG score: −4.40 | | | |
| | GvH: von Heijne's method for signal seq. recognition | | | |
| | GvH score (threshold: −2.1): −2.31 | | | |
| | possible cleavage site: between 41 and 42 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL Likelihood = −3.88 Transmembrane | | | |
| | 28-44 | | | |
| | PERIPHERAL Likelihood = 0.85 (at 322) | | | |
| | ALOM score: −3.88 (number of TMSs: 1) | | | |
| | MTOP: Prediction of membrane topology (Hartmann et al.) | | | |
| | Center position for calculation: 35 | | | |
| | Charge difference: −5.0 C(−2.0) − N( 3.0) | | | |
| | N >= C: N-terminal side will be inside | | | |
| | >>> membrane topology: type 2 (cytoplasmic tail 1 to 28) | | | |
| | MITDISC: discrimination of mitochondrial targeting seq | | | |
| | R content: | 0 | Hyd Moment(75): | 1.70 |
| | Hyd Moment(95): | 5.77 | G content: | 1 |
| | D/E content: | 2 | S/T content: | 4 |

Score: −6.97
Gavel: prediction of cleavage sites for mitochondrial
preseq
cleavage site motif not found
NUCDISC: discrimination of nuclear localization signals
pat4: KPRR (4) at 464
pat7: PKPRRIL (5) at 463
bipartite: none
content of basic residues: 10.0%
NLS Score: 0.21
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 89
COIL: Lupas's algorithm to detect coiled-coil regions
total: 0 residues
--------------------------
Final Results (k = 9/23):
30.4%: cytoplasmic
30.4%: mitochondrial
13.0%: Golgi
8.7%: endoplasmic reticulum
4.3%: extracellular, including cell wall
4.3%: vacuolar
4.3%: nuclear
4.3%: vesicles of secretory system
>> prediction for CG140122-07 is cyt (k = 23)

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAB73670 | Human oxidoreductase | 2 . . . 556 | 555/555 (100%) | 0.0 |
| | protein ORP-3 - Homo | 1 . . . 555 | 555/555 (100%) | |
| | sapiens, 555 aa. | | | |
| | [WO200144448-A2, | | | |
| | 21 JUN 2001] | | | |
| AAB12164 | Hydrophobic domain protein | 2 . . . 556 | 555/555 (100%) | 0.0 |
| | from clone HP10673 isolated | 1 . . . 555 | 555/555 (100%) | |
| | from Thymus cells - Homo | | | |
| | sapiens, 555 aa. | | | |
| | [WO200029448-A2, | | | |
| | 25 MAY 2000] | | | |
| AAM79546 | Human protein SEQ ID NO | 2 . . . 511 | 509/510 (99%) | 0.0 |
| | 3192 - Homo sapiens, 518 | 7 . . . 516 | 509/510 (99%) | |
| | aa. [WO200157190-A2, | | | |
| | 09 AUG 2001] | | | |
| AAM78562 | Human protein SEQ ID NO | 2 . . . 511 | 502/511 (98%) | 0.0 |
| | 1224 - Homo sapiens, 513 | 1 . . . 511 | 502/511 (98%) | |
| | aa. [WO200157190-A2, | | | |
| | 09 AUG 2001] | | | |
| AAU21643 | Novel human neoplastic | 274 . . . 556 | 283/283 (100%) | e−173 |
| | disease associated | 53 . . . 335 | 283/283 (100%) | |
| | polypeptide #76 - Homo | | | |
| | sapiens, 335 aa. | | | |
| | [WO200155163-A1, | | | |
| | 02 AUG 2001] | | | |

In a BLAST search of public sequence datbases, the NOV4a protein was found to have homology to the proteins shown in the BLASTP data in Table 4E.

TABLE 4E
Public BLASTP Results for NOV4a

| | | NOV4a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9NWM0 | Hypothetical protein | 2 . . . 556 | 555/555 (100%) | 0.0 |
| | FLJ20746 - Homo sapiens | 1 . . . 555 | 555/555 (100%) | |
| | (Human), 555 aa. | | | |
| Q96QT3 | Polyamine oxidase | 2 . . . 556 | 554/555 (99%) | 0.0 |
| | isoform-1 - Homo sapiens | 1 . . . 555 | 554/555 (99%) | |
| | (Human), 555 aa. | | | |
| Q99K82 | Similar to hypothetical | 2 . . . 555 | 529/554 (95%) | 0.0 |
| | protein - Mus musculus | 1 . . . 554 | 538/554 (96%) | |
| | (Mouse), 555 aa. | | | |
| Q9NP51 | DJ779E11.1.5 (Novel flavin | 145 . . . 556 | 412/412 (100%) | 0.0 |
| | containing amine oxidase | 1 . . . 412 | 412/412 (100%) | |
| | (Translation of cDNA | | | |
| | DFKZp761P0724 | | | |
| | (Em: AL162058)) (Isoform | | | |
| | 5)) - Homo sapiens (Human), | | | |
| | 412 aa (fragment). | | | |
| Q9H6H1 | Hypothetical protein | 198 . . . 556 | 358/389 (92%) | 0.0 |
| | FLJ22285 - Homo sapiens | 1 . . . 389 | 358/389 (92%) | |
| | (Human), 389 aa. | | | |

PFam analysis predicts that the NOV4a protein contains the domains shown in the Table 4F.

TABLE 4F
Domain Analysis of NOV4a

| Pfam | NOV4a | Identities/Similarities | Expect |
|---|---|---|---|
| Domain | Match Region | for the Matched Region | Value |
| FAD_binding_3 | 28 . . . 142 | 24/142 (17%) | 0.31 |
| | | 74/142 (52%) | |
| Amino_oxidase | 35 . . . 545 | 124/574 (22%) | 2.3e−28 |
| | | 365/574 (64%) | |

### Example 5

The NOV5 clone was analyzed, and the nucleotide and encoded polypeptide sequences are shown in Table 5A.

TABLE 5A
NOV5 Sequence Analysis

| | SEQ ID NO:81 | 1122 bp | |
|---|---|---|---|
| NOV5a, | ACAGACAGCCGCATCTTCTTGTGCTTTGCCAGCCACGTACGTCCCTGAGACACCACAGTGAAGTTGAA | | |
| CG141051-01 | | | |
| DNA Sequence | GTCCGGAGTCAATGGATTTGTTGGAACCGGGAGCCTAGTCACCAGGGCTGTTTTTAACTCTAGTAAAG | | |
| TAGATATTGTTGCCATCAATGACCCCTTCATTGACCTCAACTACATGGTCTACATGTTCCAGTATGAT | | | |
| | TCCACCCATGGCAAATTCCATGGCACCATCAAGGCTGAGAACGGGAAGCTTGTCATCAATGGAAATCC | | |
| | CATCACCATTTTCGAGGTGCGAGACCCCTCCAAAATCAAATGGGGCAATGCTGGTGCTGAGTCCATCA | | |
| | TGGAGTCGACCGGCATCTTCACCACCAGGAAGAAGGCTGGGGCTCGCTTGCAGGGAGGAGCCAAAAGG | | |
| | GTTATCATCTCTGCCCCCTCTTCTGACGCTCCCATGTTCGTGATGGGCATGAACCACGAGAAGTATGA | | |
| | CAACAGCTTCAAGATCATCAGCAATGCCTCCTGCACCACCAACTGCCTGGCCCCAGCCAAGGTCACCC | | |
| | ATGACAACTTTGGTATCGTGGAAGGACTCATGACCACAGTCCACGCCATTACTGCCACCCAGAAGACT | | |
| | GTGGATGGCCCCTCCGGGAAACTGTGGCATCATGGCCGTCGGACTCTCCAGAATATCATCCGTGCCTC | | |
| | TACTGGCACTGCCAAGGCTGTAGGCAAGGTCATCCTTGAGCTGAATGGGAAGCTCATAGGCATGGCCT | | |
| | TCCATATCCCCACTGCCAACGTGTTGGTCATGGACCTGACCTGCCATCTGGGAAAACCCTGCCAAGCC | | |
| | AAATATGATGATGTCAAGAAAATGATGAAGCAGGCATTGGAGGACCCCCTCAAGGGCATCCTGGGCCA | | |
| | CAGTGAGCACCAGGTCGTCTCCTCTGACTTCGACAGCGACACCCACTCTTCCACCTTCAATGCTGGGG | | |
| | CTGGCATTGCCCTCAACAACCACTTTGTGAAGCTCATTTCCTGGTATGACGATGAATTTGGCTACAGC | | |
| | AACAGTATGGTGGACCTCATGGCCCACATGGCCTCCAAGGAGTAAGACCCCCAGACCACCAGCTCCAG | | |
| | AGAGAGCATGAGAGGAACAGAGAGGTCCTCACTG | | |
| | ORF Start: ATG at 181 | | ORF Stop: TAA at 1063 |
| | SEQ ID NO:82 | 294 aa | MW at 32015.4 kD |

| | |
|---|---|
| NOV5a, | MVYMFQYDSTHGKFHGTIKAENGKLVINGNPITIFEVRDPSKIKWGNAGAESIMESTGIFTTRKKAGA |
| CG141051-01 | |
| Protein | RLQGGAKRVIISAPSSDAPMFVMGMNHEKYDNSFKIISNASCTTNCLAPAKVTHDNFGIVEGLMTTVH |
| Sequence | |
| | AITATQKTVDGPSGKLWHHGRRTLQNIIRASTGTAKAVGKVILELNGKLIGMAFHIPTANVLVMDLTC |
| | HLGKPCQAKYDDVKKMMKQALEDPLKGILGHSEHQVVSSDFDSDTHSSTFNAGAGIALNNHFVKLISW |
| | YDDEFGYSNSMVDLMAHMASKE |

Further analysis of the NOV5a protein yielded the following properties shown in Table 5B.

TABLE 5B
Protein Sequence Properties NOV5a

| | |
|---|---|
| SignalP analysis: | No Known Signal Sequence Predicted |
| PSORTII analysis: | PSG: a new signal peptide prediction method |
| | N-region: length 8; pos.chg 0; neg.chg 1 |
| | H-region: length 4; peak value 0.00 |
| | PSG score: −4.40 |
| | GvH: von Heijne's method for signal seq. recognition |
| | GvH score (threshold: −2.1) −11.52 |
| | possible cleavage site: between 61 and 62 |
| | >>> Seems to have no N-terminal signal peptide |
| | ALOM: Klein et al's method for TM region allocation |
| | Init position for calcculation: 1 |
| | Tentative number of TMS(s) for the threshold 0.5: 0 |
| | number of TMS(s) . . . fixed |
| | PERIPHERAL Likelihood = 2.44 (at 185) |
| | ALOM score: 2.44 (number of TMSs: 0) |
| | MITDISC: discrimination of mitochondrial targeting seq |

| | | | |
|---|---|---|---|
| R content: | 0 | Hyd Moment(75): | 6.17 |
| Hyd Moment(95): | 3.75 | G content: | 2 |
| D/E content: | 2 | S/T content: | 3 |
| Score: −7.36 | | | |

```
Gavel: prediction of cleavage sites for mitochondrial preseq
cleavage site motif not found
NUCDISC: discrimination of nuclear localization signals
pat4: none
pat7: none
bipartite: none
content of basic residues: 10.5%
NLS Score: −0.47
KDEL: ER retention motif in the C-terminus: none
ER Membrane Retention Signals:
KKXX-like motif in the C-terminus: MASK
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasnic/Nuclear discrimination
Prediction: cytoplasmic
Reliability: 89
COIL: Lupas's algorithm to detect coiled-coil regions
total: 0 residues
Final Results (k = 9/23):
69.6 %: cytoplasmic
  8.7 %: mitochondrial
  8.7 %: nuclear
  4.3 %: vacuolar
  4.3 %: plasma membrane
  4.3 %: peroxisomal
> prediction for CG1410S1-01 is cyt (k = 23)
```

A search of the NOV5a protein against the Geneseq database, a proprietary database that contains sequences published in patents and patent publication, yielded several homologous proteins shown in Table 5C.

TABLE 5C
Geneseq Results for NOV5a

| | | | Identities/ | |
|---|---|---|---|---|
| | | | Similarities for | |
| Geneseq | Protein/Organism/Length | NOV5a Residues/ | the Matched | Expect |
| Identifier | [Patent #, Date] | Match Residues | Region | Value |
| ABP65145 | Hypoxia-regulated protein | 1 . . . 294 | 250/295 (84%) | e−143 |
| | #19 - Homo sapiens, 335 aa. | 43 . . . 335 | 271/295 (91%) | |
| | [WO200246465-A2, | | | |
| | 13-JUN-2002] | | | |
| AAY05368 | Human HCMV inducible | 1 . . . 294 | 250/295 (84%) | e−143 |
| | gene protein, SEQ ID NO 4 - | 43 . . . 335 | 271/295 (91%) | |
| | Homo sapiens, 335 aa. | | | |
| | [WO9913075-A2, | | | |
| | 18-MAR-1999] | | | |
| AAY07036 | Breast cancer associated | 1 . . . 294 | 250/295 (84%) | e−143 |
| | antigen precursor sequence - | 43 . . . 335 | 271/295 (91%) | |
| | Homo sapiens, 335 aa. | | | |
| | [WO9904265-A2, | | | |
| | 28-JAN-1999] | | | |
| ABG13650 | Novel human diagnostic | 1 . . . 294 | 248/295 (84%) | e−141 |
| | protein #13641 - Homo | 65 . . . 357 | 268/295 (90%) | |
| | sapiens, 357 aa | | | |
| | [WO200175067-A2, | | | |
| | 11-OCT-2001] | | | |
| ABG13646 | Novel human diagnostic | 1 . . . 294 | 248/295 (84%) | e−141 |
| | protein #13637 - Homo | 65 . . . 357 | 268/295 (90%) | |
| | sapiens, 357 aa. | | | |
| | [WO200175067-A2, | | | |
| | 11-OCT-2001] | | | |

In a BLAST search of public sequence datbases, the NOV5a protein was found to have homology to the proteins shown in the BLASTP data in Table 5D.

TALBE 5D
Public BLASTP Results for NOV5a

| | | NOV5a | | |
|---|---|---|---|---|
| Protein | | Residues/ | Identities/Similarities | |
| Accession | | Match | for the | Expect |
| Number | Protein/Organism/Length | Residues | Matched Portion | Value |
| AAH23632 | Similar to | 1 . . . 294 | 250/295 (84%) | e−143 |
| | glyceraldehyde-3-phosphate | 43 . . . 335 | 271/295 (91%) | |
| | dehydrogenase - Homo | | | |
| | sapiens (Human), 335 aa. | | | |
| P04406 | Glyceraldehyde-3-phosphate | 1 . . . 294 | 250/295 (84%) | e−143 |
| | dehydrogenase, liver (EC | 42 . . . 334 | 271/295 (91%) | |
| | 1.2.1.12) - Homo sapiens | | | |
| | (Human), 334 aa. | | | |
| Q9N2D5 | Glyceraldehyde-3-phosphate | 1 . . . 294 | 239/295 (81%) | e−136 |
| | dehydrogenase (EC 1.2.1.12) | 41 . . . 333 | 264/295 (89%) | |
| | (GAPDH) - Felis silvestris | | | |
| | catus (Cat), 333 aa. | | | |
| P00355 | Glyceraldehyde 3-phosphate | 1 . . . 294 | 237/295 (80%) | e−135 |
| | dehydrogenase (EC 1.2.1.12) | 40 . . . 332 | 262/295 (88%) | |
| | (GAPDH) - Sus scrofa (Pig), | | | |
| | 332 as. | | | |
| Q9QWU4 | Glyceraldehyde 3-phosphate | 1 . . . 294 | 235/295 (79%) | e−134 |
| | dehydrogenase (EC 1.2.1.12) | 41 . . . 333 | 265/295 (89%) | |
| | (GAPDH) - Rattus norvegicus | | | |
| | (Rat), 333 aa. | | | |

PFam analysis predicts that the NOV5a protein contains the domains shown in the Table 5E.

TABLE 5E
Domain Analysis of NOV5a

| Pfam | NOV5a | Identities/Similarities | Expect |
|---|---|---|---|
| Domain | Match Region | for the Matched Region | Value |
| gpdh | 1 . . . 110 | 60/135 (44%) | 4e−85 |
| | | 103/135 (76%) | |
| gpdh_C | 111 . . . 273 | 100/178 (56%) | 1.4e−78 |
| | | 135/178 (76%) | |

### Example 6

The NOV6 clone was analyzed, and the nucleotide and encoded polypeptide sequences are shown in Table 6A.

TABLE 6A
NOV6 Sequence Analysis

| | SEQ ID NO:83 | 3368 bp | |
|---|---|---|---|
| NOV6a, | CCCGGTCCGAAGCGCGCGGATTCCACCATGTCGGCCAAGGCAATTTCAGAGCAGACGGGCAAAGAACT | | |
| CG142427-05 | | | |
| DNA Sequence | CCTTTACAAGTTCATCTGTACCACCTCAGCCATCCAGAATCGGTTCAAGTATGCTCGGGTCACTCCTG | | |
| | ACACAGACTGGGCCCGCTTGCTGCAGGACCACCCCTGGCTGCTCAGCCAGAACTTGGTAGTCAAGCCA | | |
| | GACCAGCTGATCAAACGTCGTGGAAAACTTGGTCTCGTTGGGGTCAACCTCACTCTGGATGGGGTCAA | | |
| | GTCCTGGCTGAAGCCACGGCTGGGACAGGAAGCCACAGTTGGCAAGGCCACAGGCTTCCTCAAGAACT | | |
| | TTCTGATCGAGCCCTTCGTCCCCCACAGTCAGGCTGAGGAGTTCTATGTCTGCATCTATGCCACCCGA | | |
| | GAAGGGGACTACGTCCTGTTCCACCACGAGGGGGGTGTGGACGTGGGTGATGTGGACGCCAAGGCCCA | | |
| | GAAGCTGCTTGTTGGCGTGGATGAGAAACTGAATCCTGAGGACATCAAAAAACACCTGTTGGTCCACG | | |
| | CCCCTGAAGACAAGAAAGAAATTCTGGCCAGTTTTATCTCCGGCCTCTTCAATTTCTACGAGGACTTG | | |
| | TACTTCACCTACCTCGAGATCAATCCCCTTGTAGTGACCAAAGATGGAGTCTATGTCCTTGACTTGGC | | |
| | GGCCAAGGTGGACGCCACTGCCGACTACATCTGCAAAGTGAAGTGGGGTGACATCGAGTTCCCTCCCC | | |
| | CCTTCGGGCGGGAGGCATATCCAGAGGAAGCCTACATTGCAGACCTCGATGCCAAAAGTGGGGCAAGC | | |
| | CTGAAGCTGACCTTGCTGAACCCCAAAGGGAGGATCTGGACCATGGTGGCCGGGGGTGGCGCCTCTGT | | |
| | CGTGTACAGCGATACCATCTGTGATCTAGGGGGTGTCAACGAGCTGGCAAACTATGGGGAGTACTCAG | | |
| | GCGCCCCCAGCGAGCAGCAGACCTATGATTATGCCAAGACTATCCTCTCCCTCATGACCCGAGAGAAG | | |
| | CACCCAGATGGCAAGATCCTCATCATTGGAGGCAGCATCGCAAACTTCACCAACGTGGCTGCCACGTT | | |
| | CAAGGGCATCGTGAGAGCAATTCGAGATTACCAGGGCCCCCTGAAGGAGCACGAAGTCACAATCTTTG | | |
| | TCCGAAGAGGTGGCCCCAACTATCAGGAGGGCTTACGGGTGATGGGAGAAGTCGGGAAGACCACTGGG | | |
| | ATCCCCATCCATGTCTTTGGCACAGAGACTCACATGACGGCCATTGTGGGCATGGCCCTGGGCCACCG | | |
| | GCCCATCCCCAACCAGCCACCCACAGCGGCCCACACTGCAAACTTCCTCCTCAACGCCAGCGGGAGCA | | |
| | CATCGACGCCAGCCCCCAGCAGGACAGCATCTTTTTCTGAGTCCAGGGCCGATGAGGTGGCGCCTGCA | | |
| | AAGAAGGCCAAGCCTGCCATGCCACAAGATTCAGTCCCAAGTCCAAGATCCCTGCAAGGAAAGAGCAC | | |
| | CACCCTCTTCAGCCGCCACACCAAGGCCATTGTGTGGGGCATGCAGACCCGGGCCGTGCAAGGCATGC | | |
| | TGGACTTTGACTATGTCTGCTCCCGAGACGAGCCCTCAGTGGCTGCCATGGTCTACCCTTTCACTGGG | | |
| | GACCACAAGCAGAAGTTTTACTGGGGGCACAAAGAGATCCTGATCCCTGTCTTCAAGAACATGGCTGA | | |
| | TGCCATGAGGAAGCACCCGGAGGTAGATGTGCTCATCAACTTTGCCTCTCTCCGCTCTGCCTATGACA | | |
| | GCACCATGGAGACCATGAACTATGCCCAGATCCGGACCATCGCCATCATAGCTGAAGGCATCCCTGAG | | |
| | GCCCTCACGAGAAAGCTGATCAAGAAGGCGGACCAGAAGGGAGTGACCATCATCGGACCTGCCACTGT | | |
| | TGGAGGCATCAAGCCTGGGTGCTTTAAGATTGGCAACACAGGTGGGATGCTGGACAACATCCTGGCCT | | |
| | CCAAACTGTACCGCCCAGGCAGCGTGGCCTATGTCTCACGTTCCGGAGGCATGTCCAACGAGCTCAAC | | |
| | AATATCATCTCTCGGACCACGGATGGCGTCTATGAGGGCGTGGCCATTGGTGGGGACAGGTACCCGGG | | |
| | CTCCACATTCATGGATCATGTGTTACGCTATCAGGACACTCCAGGAGTCAAAATGATTGTGGTTCTTG | | |
| | GAGAGATTGGGGGCACTGAGGAATATAAGATTTGCCGGGGCATCAAGGAGGGCCGCCTCACTAAGCCC | | |
| | ATCGTCTGCTGGTGCATCGGGACGTGTGCCACCATGTTCTCCTCTGAGGTCCAGTTTGGCCATGCTGG | | |
| | AGCTTGTGCCAACCAGGCTTCTGAAACTGCAGTAGCCAAGAACCAGGCTTTGAAGGAAGCAGGAGTGT | | |
| | TTGTGCCCCGGAGCTTTGATGAGCTTGGAGAGATCATCCAGTCTGTATACGAAGATCTCGTGGCCAAT | | |
| | GGAGTCATTGTACCTGCCCAGGAGGTGCCGCCCCCAACCGTGCCCATGGACTACTCCTGGGCCAGGGA | | |
| | GCTTGGTTTGATCCGCAAACCTGCCTCGTTCATGACCAGCATCTGCGATGAGCGAGGACAGGAGCTCA | | |
| | TCTACGCGGGCATGCCCATCACTGAGGTCTTCAAGGAAGAGATGGGCATTGGCGGGGTCCTCGGCCTC | | |
| | CTCTGGTTCCAGAAAAGGTTGCCTAAGTACTCTTGCCAGTTCATTGAGATGTGTCTGATGGTGACAGC | | |
| | TGATCACGGGCCAGCCGTCTCTGGAGCCCACAACACCATCATTTGTGCGCGAGCTGGGAAAGACCTGG | | |
| | TCTCCAGCCTCACCTCGGGGCTGCTCACCATCGGGGATCGGTTTGGGGGTGCCTTGGATGCAGCAGCC | | |
| | AAGATGTTCAGTAAAGCCTTTGACAGTGGCATTATCCCCATGGAGTTTGTGAACAAGATGAAGAAGGA | | |
| | AGGGAAGCTGATCATGGGCATTGGTCACCGAGTGAAGTCGATAAACAACCCAGACATGCGAGTGCAGA | | |
| | TCCTCAAAGATTACGTCAGGCAGCACTTCCCTGCCACTCCTCTGCTCGATTATGCACTGGAAGTAGAG | | |
| | AAGATTACCACCTCGAAGAAGCCAAATCTTATCCTGAATGTAGATGGTCTCATCGGAGTCGCATTTGT | | |
| | AGACATGCTTAGAAACTGTGGGTCCTTTACTCGGGAGGAAGCTGATGAATATATTGACATTGGAGCCC | | |
| | TCAATGGCATCTTTGTGCTGGGAAGGAGTATGGGGTTCATTGGACACTATCTTGATCAGAAGAGGCTG | | |
| | AAGCAGGGGCTGTATCGTCATCCGTGGGATGATATTTCATATGTTCTTCCGGAACACATGAGCATGCA | | |
| | TCATCACCACCATCACTAAGCGGCCGCTTTCGAATC | | |
| | ORF Start: ATG at 28 | | ORF Stop: at 3331 |
| | SEQ ID NO:84 | 1101 aa | MW at 120838.0 kD |
| NOV6a, | MSAKAISEQTGKELLYKFICTTSAIQNRFKYARVTPDTDWARLLQDHPWLLSQNLVVKPDQLIKRRGK | | |
| CG142427-05 | | | |
| Protein | LGLVGVNLTLDGVKSWLKPRLGQEATVGKATGFLKNFLIEPFVPHSQAEEFYVCIYATREGDYVLFHH | | |
| Sequence | | | |
| | EGGVDVGDVDAKAQKLLVGVDEKLNPEDIKKHLLVHAPEDKKEILASFISGLFNFYEDLYFTYLEINP | | |
| | LVVTKDGVYVLDLAAKVDATADYICKVKWGDIEFPPPFGREAYPEEAYIADLDAKSGASLKLTLLNPK | | |
| | GRIWTMVAGGGASVVYSDTICDLGGVNELANYGEYSGAPSEQQTYDYAKTILSLMTREKHPDGKILII | | |
| | GGSIANFTNVAATFKGIVRAIRDYQGPLKEHEVTIFVRRGGPNYQEGLRVMGEVGKTTGIPIHVFGTE | | |
| | THMTAIVGMALGHRPIPNQPPTAAHTANFLLNASGSTSTPAPSRTASFSESRADEVAPAKKAKPAMPQ | | |
| | DSVPSPRSLQGKSTTLFSRHTKAIVWGMQTRAVQGMLDFDYVCSRDEPSVAAMVYPFTGDHKQKFYWG | | |
| | HKEILIPVFKNMADAMRKHPEVDVLINFASLRSAYDSTMETMNYAQIRTIAIIAEGIPEALTRKLIKK | | |
| | ADQKGVTIIGPATVGGIKPGCFKIGNTGGMLDNILASKLYRPGSVAYVSRSGGMSNELNNIISRTTDG | | |
| | VYEGVAIGGDRYPGSTFMDHVLRYQDTPGVKMIVVLGEIGGTEEYKICRGIKEGRLTKPIVCWCIGTC | | |
| | ATMFSSEVQFGHAGACANQASETAVAKNQALKEAGVFVPRSFDELGEIIQSVYEDLVANGVIVPAQEV | | |
| | PPPTVPMDYSWARELGLIRKPASFMTSICDERGQELIYAGMPITEVFKEEMGIGGVLGLLWFQKRLPK | | |
| | YSCQFIEMCLMVTADHGPAVSGAHNTIICARAGKDLVSSLTSGLLTIGDRFGGALDAAAKMFSKAFDS | | |
| | GIIPMEFVNKMKKEGKLIMGIGHRVKSINNPDMRVQILKDYVRQHFPATPLLDYALEVEKITTSKKPN | | |
| | LILNVDGLIGVAFVDMLRNCGSFTREEADEYIDIGALNGIFVLGRSMGFIGHYLDQKRLKQGLYRHPW | | |
| | DDISYVLPEHMSM | | |
| | SEQ ID NO:85 | 3307 bp | |
| NOV6b, | CCAGAATTCCACCATGTCGGCCAAGGCAATTTCAGAGCAGACGGGCAAAGAACTCCTTTACAAGTTC | | |
| CG142427-02 | | | |
| DNA Sequence | ATCTGTACCACCTCAGCCATCCAGAATCGGTTCAAGTATGCTCGGGTCACTCCTGACACAGACTGGG | | |
| | CCCGCTTGCTGCAGGACCACCCCTGGCTGCTCAGCCAGAACTTGGTAGTCAAGCCAGACCAGCTGAT | | |
| | CAAACGTCGTGGAAAACTTGGTCTCGTTGGGGTCAACCTCACTCTGGATGGGGTCAAGTCCTGGCTG | | |
| | AAGCCACGGCTGGGACAGGAAGCCACAGTTGGCAAGGCCACAGGCTTCCTCAAGAACTTTCTGATCG | | |
| | AGCCCTTCGTCCCCCACAGTCAGGCTGAGGAGTTCTATGTCTGCATCTATGCCACCCGAGAAGGGGA | | |
| | CTACGTCCTGTTCCACCACGAGGGGGGTGTGGACGTGGGTGATGTGGACGCCAAGGCCCAGAAGCTG | | |
| | CTTGTTGGCGTGGATGAGAAACTGAATCCTGAGGACATCAAAAAACACCTGTTGGTCCACGCCCCTG | | |
| | AAGACAAGAAAGAAATTCTGGCCAGTTTTATCTCCGGCCTCTTCAATTTCTACGAGGACTTGTACTT | | |
| | CACCTACCTCGAGATCAATCCCCTTGTAGTGACCAAAGATGGAGTCTATGTCCTTGACTTGGCGGCC | | |
| | AAGGTGGACGCCACTGCCGACTACATCTGCAAAGTGAAGTGGGGTGACATCGAGTTCCCTCCCCCCT | | |
| | TCGGGCGGGAGGCATATCCAGAGGAAGCCTACATTGCAGACCTCGATGCCAAAAGTGGGGCAAGCCT | | |
| | GAAGCTGACCTTGCTGAACCCCAAAGGGAGGATCTGGACCATGGTGGCCGGGGGTGGCGCCTCTGTC | | |
| | GTGTACAGCGATACCATCTGTGATCTAGGGGGTGTCAACGAGCTGGCAAACTATGGGGAGTACTCAG | | |
| | GCGCCCCCAGCGAGCAGCAGACCTATGACTATGCCAAGACTATCCTCTCCCTCATGACCCGAGAGAA | | |
| | GCACCCAGATGGCAAGATCCTCATCATTGGAGGCAGCATCGCAAACTTCACCAACGTGGCTGCCACG | | |
| | TTCAAGGGCATCGTGAGAGCAATTCGAGATTACCAGGGCCCCCTGAAGGAGCACGAAGTCACAATCT | | |
| | TTGTCCGAAGAGGTGGCCCCAACTATCAGGAGGGCTTACGGGTGATGGGAGAAGTCGGGAAGACCAC | | |
| | TGGGATCCCCATCCATGTCTTTGGCACAGAGACTCACATGACGGCCATTGTGGGCATGGCCCTGGGC | | |
| | CACCGGCCCATCCCCAACCAGCCACCCACAGCGGCCCACACTGCAAACTTCCTCCTCAACGCCAGCG | | |
| | GGAGCACATCGACGCCAGCCCCCAGCAGGACAGCATCTTTTTCTGAGTCCAGGGCCGATGAGGTGGC | | |
| | GCCTGCAAAGAAGGCCAAGCCTGCCATGCCACAAGGAAAGAGCACCACCCTCTTCAGCCGCCACACC | | |
| | AAGGCCATTGTGTGGGGCATGCAGACCCGGGCCGTGCAAGGCATGCTGGACTTTGACTATGTCTGCT | | |
| | CCCGAGACGAGCCCTCAGTGGCTGCCATGGTCTACCCTTTCACTGGGGACCACAAGCAGAAGTTTTA | | |
| | CTGGGGGCACAAAGAGATCCTGATCCCTGTCTTCAAGAACATGGCTGATGCCATGAGGAAGCACCCG | | |
| | GAGGTAGATGTGCTCATCAACTTTGCCTCTCTCCGCTCTGCCTATGACAGCACCATGGAGACCATGA | | |
| | ACTATGCCCAGATCCGGACCATCGCCATCATAGCTGAAGGCATCCCTGAGGCCCTCACGAGAAAGCT | | |
| | GATCAAGAAGGCGGACCAGAAGGGAGTGACCATCATCGGACCTGCCACTGTTGGAGGCATCAAGCCT | | |
| | GGGTGCTTTAAGATTGGCAACACAGGTGGGATGCTGGACAACATCCTGGCCTCCAAACTGTACCGCC | | |
| | CAGGCAGCGTGGCCTATGTCTCACGTTCCGGAGGCATGTCCAACGAGCTCAACAATATCATCTCTCG | | |
| | GACCACGGATGGCGTCTATGAGGGCGTGGCCATTGGTGGGGACAGGTACCCGGGCTCCACATTCATG | | |
| | GATCATGTGTTACGCTATCAGGACACTCCAGGAGTCAAAATGATTGTGGTTCTTGGAGAGATTGGGG | | |
| | GCACTGAGGAATATAAGATTTGCCGGGGCATCAAGGAGGGCCGCCTCACTAAGCCCATCGTCTGCTG | | |
| | GTGCATCGGGACGTGTGCCACCATGTTCTCCTCTGAGGTCCAGTTTGGCCATGCTGGAGCTTGTGCC | | |
| | AACCAGGCTTCTGAAACTGCAGTAGCCAAGAACCAGGCTTTGAAGGAAGCAGGAGTGTTTGTGCCCC | | |
| | GGAGCTTTGATGAGCTTGGAGAGATCATCCAGTCTGTATACGAAGATCTCGTGGCCAATGGAGTCAT | | |
| | TGTACCTGCCCAGGAGGTGCCGCCCCCAACCGTGCCCATGGACTACTCCTGGGCCAGGGAGCTTGGT | | |
| | TTGATCCGCAAACCTGCCTCGTTCATGACCAGCATCTGCGATGAGCGAGGACAGGAGCTCATCTACG | | |
| | CGGGCATGCCCATCACTGAGGTCTTCAAGGAAGAGATGGGCATTGGCGGGGTCCTCGGCCTCCTCTG | | |
| | GTTCCAGAAAAGGTTGCCTAAGTACTCTTGCCAGTTCATTGAGATGTGTCTGATGGTGACAGCTGAT | | |
| | CACGGGCCAGCCGTCTCTGGAGCCCACAACACCATCATTTGTGCGCGAGCTGGGAAAGACCTGGTCT | | |
| | CCAGCCTCACCTCGGGGCTGCTCACCATCGGGGATCGGTTTGGGGGTGCCTTGGATGCAGCAGCCAA | | |
| | GATGTTCAGTAAAGCCTTTGACAGTGGCATTATCCCCATGGAGTTTGTGAACAAGATGAAGAAGGAA | | |
| | GGGAAGCTGATCATGGGCATTGGTCACCGAGTGAAGTCGATAAACAACCCAGACATGCGAGTGCAGA | | |
| | TCCTCAAAGATTACGTCAGGCAGCACTTCCCTGCCACTCCTCTGCTCGATTATGCACTGGAAGTAGA | | |
| | GAAGATTACCACCTCGAAGAAGCCAAATCTTATCCTGAATGTAGATGGTCTCATCGGAGTCGCATTT | | |
| | GTAGACATGCTTAGAAACTGTGGGTCCTTTACTCGGGAGGAAGCTGATGAATATATTGACATTGGAG | | |
| | CCCTCAATGGCATCTTTGTGCTGGGAAGGAGTATGGGGTTCATTGGACACTATCTTGATCAGAAGAG | | |
| | GCTGAAGCAGGGGCTGTATCGTCATCCGTGGGATGATATTTCATATGTTCTTCCGGAACACATGAGC | | |
| | ATGTAAGCGGCCGCTTTTTTCCTT | | |
| | ORF Start: at 2 | | ORF Stop: TAA at 3287 |
| | SEQ ID NO:86 | 1095 aa | MW at 120201.2 kD |
| NOV6b, | QNSTMSAKAISEQTGKELLYKFICTTSAIQNRFKYARVTPDTDWARLLQDHPWLLSQNLVVKPDQLI | | |
| CG142427-02 | | | |
| Protein | KRRGKLGLVGVNLTLDGVKSWLKPRLGQEATVGKATGFLKNFLIEPFVPHSQAEEFYVCIYATREGD | | |
| Sequence | | | |
| | YVLFHHEGGVDVGDVDAKAQKLLVGVDEKLNPEDIKKHLLVHAPEDKKEILASFISGLFNFYEDLYF | | |
| | TYLEINPLVVTKDGVYVLDLAAKVDATADYICKVKWGDIEFPPPFGREAYPEEAYIADLDAKSGASL | | |
| | KLTLLNPKGRIWTMVAGGGASVVYSDTICDLGGVNELANYGEYSGAPSEQQTYDYAKTILSLMTREK | | |
| | HPDGKILIIGGSIANFTNVAATFKGIVRAIRDYQGPLKEHEVTIFVRRGGPNYQEGLRVMGEVGKTT | | |
| | GIPIHVFGTETHMTAIVGMALGHRPIPNQPPTAAHTANFLLNASGSTSTPAPSRTASFSESRADEVA | | |
| | PAKKAKPAMPQGKSTTLFSRHTKAIVWGMQTRAVQGMLDFDYVCSRDEPSVAAMVYPFTGDHKQKFY | | |
| | WGHKEILIPVFKNMADAMRKHPEVDVLINFASLRSAYDSTMETMNYAQIRTIAIIAEGIPEALTRKL | | |
| | IKKADQKGVTIIGPATVGGIKPGCFKIGNTGGMLDNILASKLYRPGSVAYVSRSGGMSNELNNIISR | | |
| | TTDGVYEGVAIGGDRYPGSTFMDHVLRYQDTPGVKMIVVLGEIGGTEEYKICRGIKEGRLTKPIVCW | | |
| | CIGTCATMFSSEVQFGHAGACANQASETAVAKNQALKEAGVFVPRSFDELGEIIQSVYEDLVANGVI | | |
| | VPAQEVPPPTVPMDYSWARELGLIRKPASFMTSICDERGQELIYAGMPITEVFKEEMGIGGVLGLLW | | |
| | FQKRLPKYSCQFIEMCLMVTADHGPAVSGAHNTIICARAGKDLVSSLTSGLLTIGDRFGGALDAAAK | | |
| | MFSKAFDSGIIPMEFVNKMKKEGKLIMGIGHRVKSINNPDMRVQILKDYVRQHFPATPLLDYALEVE | | |
| | KITTSKKPNLILNVDGLIGVAFVDMLRNCGSFTREEADEYIDIGALNGIFVLGRSMGFIGHYLDQKR | | |
| | LKQGLYRHPWDDISYVLPEHMSM | | |
| | SEQ ID NO:87 | 2290 bp | |
| NOV6c, | CCAGAATTCCACCATGTCGGCCAAGGCAATTTCAGAGCAGACGGGCAAAGAACTCCTTTACAAGTTCA | | |
| CG142427-03 | | | |
| DNA Sequence | TCTGTACCACCTCAGCCATCCAGAATCGGTTCAAGTATGCTCGGGTCACTCCTGACACAGACTGGGCC | | |
| | CGCTTGCTGCAGGACCACCCCTGGCTGCTCAGCCAGAACTTGGTAGTCAAGCCAGACCAGCTGATCAA | | |
| | ACGTCGTGGAAAACTTGGTCTCGTTGGGGTCAACCTCACTCTGGATGGGGTCAAGTCCTGGCTGAAGC | | |
| | CACGGCTGGGACAGGAAGCCACAGTGAGTGGGCATGGGGTCAAGATGAACGTGTGTGGTAACAGAAGC | | |
| | AAATATGGTCACCTTCAGGTTGGCAAGGCCACAGGCTTCCTCAAGAACTTTCTGATCGAGCCCTTCGT | | |
| | CCCCCACAGTCAGGCTGAGGAGTTCTATGTCTGCATCTATGCCACCCGAGAAGGGGACTACGTCCTGT | | |
| | TCCACCACGAGGGGGGTGTGGACGTGGGTGATGTGGACGCCAAGGCCCAGAAGCTGCTTGTTGGCGTG | | |
| | GATGAGAAACTGAATCCTGAGGACATCAAAAAACACCTGTTGGTCCACGCCCCTGAAGACAAGAAAGA | | |
| | AATTCTGGCCAGTTTTATCTCCGGCCTCTTCAATTTCTACGAGGACTTGTACTTCACCTACCTCGAGA | | |
| | TCAATCCCCTTGTAGTGACCAAAGATGGAGTCTATGTCCTTGACTTGGCGGCCAAGGTGGACGCCACT | | |
| | GCCGACTACATCTGCAAAGTGAAGTGGGGTGACATCGAGTTCCCTCCCCCCTTCGGGCGGGAGGCATA | | |
| | TCCAGAGGAAGCCTACATTGCAGACCTCGACGCCAAAAGTGGGGCAAGCCTGAAGCTGACCTTGCTGA | | |
| | ACCCCAAAGGGAGGATCTGGACCATGGTGGCCGGGGGTGGCGCCTCTGTCGTGTACAGCGATACCATC | | |
| | TGTGATCTAGGGGGTGTCAACGAGCTGGCAAACTATGGGGAGTACTCAGGCGCCCCCAGCGAGCAGCA | | |
| | GACCTATGACTATGCCAAGACTATCCTCTCCCTCATGACCCGAGAGAAGCACCCAGATGGCAAGATCC | | |
| | TCATCATTGGAGGCAGCATCGCAAACTTCACCAACGTGGCTGCCACGTTCAAGGGCATCGTGAGAGCA | | |
| | ATTCGAGATTACCAGGGCCCCCTGAAGGAGCACGAAGTCACAATCTTTGTCCGAAGAGGTGGCCCCAA | | |
| | CTATCAGGAGGGCTTACGGGTGATGGGAGAAGTCGGGAAGACCACTGGGATCCCCATCCATGTCTTTG | | |
| | GCACAGAGACTCACATGACGGCCATTGTGGGCATGGCCCTGGGCCACCGGCCCATCCCCAACCAGCCA | | |
| | CCCACAGCGGCCCACACTGCAAACTTCCTCCTCAACGCCAGCGGGAGCACATCGACGCCAGCCCCCAG | | |
| | CAGGACAGCATCTTTTTCTGAGTCCAGGGCCGATGAGGTGGCGCCTGCAAAGAAGGCCAAGCCTGCCA | | |
| | TGCCACAAGGAAAGAGCACCACCCTCTTCAGCCGCCACACCAAGGCCATTGTGTGGGGCATGCAGACC | | |
| | CGGGCCGTGCAAGGCATGCTGGACTTTGACTATGTCTGCTCCCGAGACGAGCCCTCAGTGGCTGCCAT | | |
| | GGTCTACCCTTTCACTGGGGACCACAAGCAGAAGTTTTACTGGGGGCACAAAGAGATCCTGATCCCTG | | |
| | TCTTCAAGAACATGGCTGATGCCATGAGGAAGCACCCGGAGGTAGATGTGCTCATCAACTTTGCTTCT | | |
| | CTCCGCTCTGCCTTGGATGCAGCAGCCAAGATGTTCAGTAAAGCCTTTGACAGTGGCATTATCCCCAT | | |
| | GGAGTTTGTGAACAAGATGAAGAAGGAAGGGAAGCTGATCATGGGCATTGGTCACCGAGTGAAGTCGA | | |
| | TAAACAACCCAGACATGCGAGTGCGGATCCTCAAAGATTACGTCAGGCAGCACTTCCCTGCCACTCCT | | |
| | CTGCTCGATTATGCACTGGAAGTAGAGAAGATTACCACCTCGAAGAAGCCAAATCTTATCCTGAATGT | | |
| | AGATGGTCTCATCGGAGTCGCATTTGTAGACATGCTTAGAAACTGTGGGTCCTTTACTCGGGAGGAAG | | |
| | CTGATGAATATATTGACATTGGAGCCCTCAATGGCATCTTTGTGCTGGGAAGGAGTATGGGGTTCATT | | |
| | GGACACTATCTTGATCAGAAGAGGCTGAAGCAGGGGCTGTATCGTCATCCGTGGGATGATATTTCATA | | |
| | TGTTCTTCCGGAACACATGAGCATGTAAGCGGCCGCTTTTTTCCTT | | |
| | ORF Start: at 2 | | ORF Stop: TAA at 2270 |
| | SEQ ID NO:88 | 756 aa | MW at 83890.7 kD |
| NOV6c, | QNSTMSAKAISEQTGKELLYKFICTTSAIQNRFKYARVTPDTDWARLLQDHPWLLSQNLVVKPDQLIK | | |
| CG142427-03 | | | |
| Protein | RRGKLGLVGVNLTLDGVKSWLKPRLGQEATVSGHGVKMNVCGNRSKYGHLQVGKATGFLKNFLIEPFV | | |
| Sequence | | | |
| | PHSQAEEFYVCIYATREGDYVLFHHEGGVDVGDVDAKAQKLLVGVDEKLNPEDIKKHLLVHAPEDKKE | | |
| | ILASFISGLFNFYEDLYFTYLEINPLVVTKDGVYVLDLAAKVDATADYICKVKWGDIEFPPPFGREAY | | |
| | PEEAYIADLDAKSGASLKLTLLNPKGRIWTMVAGGGASVVYSDTICDLGGVNELANYGEYSGAPSEQQ | | |
| | TYDYAKTILSLMTREKHPDGKILIIGGSIANFTNVAATFKGIVRAIRDYQGPLKEHEVTIFVRRGGPN | | |
| | YQEGLRVMGEVGKTTGIPIHVFGTETHMTAIVGMALGHRPIPNQPPTAAHTANFLLNASGSTSTPAPS | | |
| | RTASFSESRADEVAPAKKAKPAMPQGKSTTLFSRHTKAIVWGMQTRAVQGMLDFDYVCSRDEPSVAAM | | |
| | VYPFTGDHKQKFYWGHKEILIPVFKNMADAMRKHPEVDVLINFASLRSALDAAAKMFSKAFDSGIIPM | | |
| | EFVNKMKKEGKLIMGIGHRVKSINNPDMRVRILKDYVRQHFPATPLLDYALEVEKITTSKKPNLILNV | | |
| | DGLIGVAFVDMLRNCGSFTREEADEYIDIGALNGIFVLGRSMGFIGHYLDQKRLKQGLYRHPWDDISY | | |
| | VLPEHMSM | | |
| | SEQ ID NO:89 | 3238 bp | |
| NOV6d, | CCAGAATTCCACCATGTCGGCCAAGGCAATTTCAGAGCAGACGGGCAAAGAACTCCTTTACAAGTTC | | |
| CG142427-04 | | | |
| DNA Sequence | ATCTGTACCACCTCAGCCATCCAGAATCGGTTCAAGTATGCTCGGGTCACTCCTGACACAGACTGGG | | |
| | CCCGCTTGCTGCAGGACCACCCCTGGCTGCTCAGCCAGAACTTGGTAGTCAAGCCAGACCAGCTGAT | | |
| | CAAACGTCGTGGAAAACTTGGTCTCGTTGGGGTCAACCTCACTCTGGATGGGGTCAAGTCCTGGCTG | | |
| | AAGCCACGGCTGGGACAGGAAGCCACAGTTGGCAAGGCCACAGGCTTCCTCAAGAACTTTCTGATCG | | |
| | AGCCCTTCGTCCCCCACAGTCAGGCTGAGGAGTTCTATGTCTGCATCTATGCCACCCGAGAAGGGGA | | |
| | CTACGTCCTGTTCCACCACGAGGGGGGTGTGGACGTGGGTGATGTGGACGCCAAGGCCCAGAAGCTG | | |
| | CTTGTTGGCGTGGATGAGAAACTGAATCCTGAGGACATCAAAAAACACCTGTTGGTCCACGCCCCTG | | |
| | AAGACAAGAAAGAAATTCTGGCCAGTTTTATCTCCGGCCTCTTCAATTTCTACGAGGACTTGTACTT | | |
| | CACCTACCTCGAGATCAATCCCCTTGTAGTGACCAAAGATGGAGTCTATGTCCTTGACTTGGCGGCC | | |
| | AAGGTGGACGCCACTGCCGACTACATCTGCAAAGTGAAGTGGGGTGACATCGAGTTCCCTCCCCCCT | | |
| | TCGGGCGGGAGGCATATCCAGAGGAAGCCTACATTGCAGGCCTCGATGCCAAAAGTGGGGCAAGCCT | | |
| | GAAGCTGACCTTGCTGAACCCCAAAGGGAGGATCTGGACCATGGTGGCCGGGGGTGGCGCCTCTGTC | | |
| | GTGTACAGCGATACCATCTGTGATCTAGGGGGTGTCAACGAGCTGGCAAACTATGGGGAGTACTCAG | | |
| | GCGCCCCCAGCGAGCAGCAGACCTATGACTATGCCAAGACTATCCTCTCCCTCATGACCCGAGAGAA | | |
| | GCACCCAGATGGCAAGATCCTCATCATTGGAGGCAGCATCGCAAACTTCACCAACGTGGCTGCCACG | | |
| | TTCAAGGGCATCGTGAGAGCAATTCGAGATTACCAGGGCCCCCTGAAGGAGCACGAAGTCACAATCT | | |
| | TTGTCCGAAGAGGTGGCCCCAACTATCAGGAGGGCTTACGGGTGATGGGAGAAGTCGGGAAGACCAC | | |
| | TGGGATCCCCATCCATGTCTTTGGCACAGAGACCCACACTGCAAACTTCCTCCTCAACGCCAGCGGG | | |
| | AGCACATCGACGCCAGCCCCCAGCAGGACAGCATCTTTTTCTGAGTCCAGGGCCGATGAGGTGGCGC | | |
| | CTGCAAAGAAGGCCAAGCCTGCCATGCCACAAGGAAAGAGCACCACCCTCTTCAGCCGCCACACCAA | | |
| | GGCCATTGTGTGGGGCATGCAGACCCGGGCCGTGCAAGGCATGCTGGACTTTGACTATGTCTGCTCC | | |
| | CGAGACGAGCCCTCAGTGGCTGCCATGGTCTACCCTTTCACTGGGGACCACAAGCAGAAGTTTTACT | | |
| | GGGGGCACAAAGAGATCCTGATCCCTGTCTTCAAGAACATGGCTGATGCCATGAGGAAGCATCCGGA | | |
| | GGTAGATGTGCTCATCAACTTTGCCTCTCTCCGCTCTGCCTATGACAGCACCATGGAGACCACGAAC | | |
| | TATGCCCAGATCCGGACCATCGCCATCATAGCTGAAGGCATTCCTGAGGCCCTCACGAGAAAGCTGA | | |
| | TCAAGAAGGCGGACCAGAAGGGAGTGACCATCATCHHACCTGCCACTGTTGGAGGCATCAAGCCTGG | | |
| | GTGCTTTAAGATTGGCAACACAGGTGGGATGCTGGACAACATCCTGGCCTCCAAACTGTACCGCCCA | | |
| | GGCAGCGTGGCCTATGCCTCACGTTCCGGAGGCATGTCCAACGAGCTCAACAATATCATCTCTCGGA | | |
| | CCACGGATGGCGTCTATGAGGGCGTGGCCATTGGTGGGGACAGGTACCCGGGCTCCACATTCATGGA | | |
| | TCATGTGTTACGCTATCAGGACACTCCAGGAGTCAAAATGATTGTGGTTCTTGGAGAGATTGGGGGC | | |
| | ACTGAGGAATATAAGATTTGCCGGGGCATCAAGGAGGGCCGCCTCACTAAGCCCATCGTCTGCTGGT | | |
| | GCATCGGGACGTGTGCCACCATGTTCTCCTCTGAGGTCCAGTTTGGCCATGCTGGAGCTTGTGCCAA | | |
| | CCAGGCTTCTGAAACTGCAGTAGCCAAGAACCAGGCTTTGAAGGAAGCAGGAGTGTTTGTGCCCCGG | | |
| | AGCTTTGATGAGCTTGGAGAGATCATCCAGTCTGTATACGAAGATCTCGTGGCCAATGGAGTCATTG | | |
| | TACCTGCCCAGGAGGTGCCGCCCCCAACCGTGCCCATGGACTACTCCTGGGCCAGGGAGCTTGGTTT | | |
| | GATCCGCAAACCTGCCTCGTTCATGACCAGCATCTGCGATGAGCGAGGACAGGAGCTCATCTACGCG | | |
| | GGCATGCCCATCACTGAGGTCTTCAAGGAAGAGATGGGCATTGGCGGGGTCCTCGGCCTCCTCTGGT | | |
| | TCCAGAAAAGGTTGCCTAAGTACTCTTGCCAGTTCATTGAGATGTGTCTGATGGTGACAGCTGATCA | | |
| | CGGGCCAGCCGTCTCTGGAGCCCACAACACCATCATTTGTGCGCGAGCTGGGAAAGACCTGGTCTCC | | |
| | AGCCTCACCTCGGGGCTGCTCACCATCGGGGATCGGTTTGGGGGTGCCTTGGATGCAGCAGCCAAGA | | |
| | TGTTCAGTAAAGCCTTTGACAGTGGCATTATCCCCATGGAGTTTGTGAACAAGATGAAGAAGGAAGG | | |
| | GAAGCTGATCATGGGCATTGGTCACCGAGTGAAGTCGATAAACAACCCAGACATGCGAGTGCAGATC | | |
| | CTCAAAGATTACGTCAGGCAGCACTTCCCTGCCACTCCTCTGCTCGATTATGCACTGGAAGTAGAGA | | |
| | AGATTACCACCTCGAAGAAGCCAAATCTTATCCTGAATGTAGATGGTCTCATCGGAGTCGCATTTGT | | |
| | AGACATGCTTAGAAACTGTGGGTCCTTTACTCGGGAGGAAGCTGATGAATATATTGACATTGGAGCC | | |
| | CTCAATGGCATCTTTGTGCTGGGAAGGAGTATGGGGTTCATTGGACACTATCTTGATCAGAAGAGGC | | |
| | TGAAGCAGGGGCTGTATCGTCATCCGTGGGATGATATTTCATATGTTCTTCCGGAACACATGAGCAT | | |
| | GTAAGCGGCCGCTTTTTTCCTT | | |
| | ORF Start: at 2 | | ORF Stop: TAA at 3218 |
| | SEQ ID NO:90 | 1072 aa | MW at 117722.3 kD |
| NOV6d, | QNSTMSAKAISEQTGKELLYKFICTTSAIQNRFKYARVTPDTDWARLLQDHPWLLSQNLVVKPDQLI | | |
| CG142427-04 | | | |
| Protein | KRRGKLGLVGVNLTLDGVKSWLKPRLGQEATVGKATGFLKNFLIEPFVPHSQAEEFYVCIYATREGD | | |
| Sequence | | | |
| | YVLFHHEGGVDVGDVDAKAQKLLVGVDEKLNPEDIKKHLLVHAPEDKKEILASFISGLFNFYEDLYF | | |
| | TYLEINPLVVTKDGVYVLDLAAKVDATADYICKVKWGDIEFPPPFGREAYPEEAYIAGLDAKSGASL | | |
| | KLTLLNPKGRIWTMVAGGGASVVYSDTICDLGGVNELANYGEYSGAPSEQQTYDYAKTILSLMTREK | | |
| | HPDGKILIIGGSIANFTNVAATFKGIVRAIRDYQGPLKEHEVTIFVRRGGPNYQEGLRVMGEVGKTT | | |
| | GIPIHVFGTETHTANFLLNASGSTSTPAPSRTASFSESRADEVAPAKKAKPAMPQGKSTTLFSRHTK | | |
| | AIVWGMQTRAVQGMLDFDYVCSRDEPSVAAMVYPFTGDHKQKFYWGHKEILIPVFKNMADAMRKHPE | | |
| | VDVLINFASLRSAYDSTMETTNYAQIRTIAIIAEGIPEALTRKLIKKADQKGVTIIGPATVGGIKPG | | |
| | CFKIGNTGGMLDNILASKLYRPGSVAYASRSGGMSNELNNIISRTTDGVYEGVAIGGDRYPGSTFMD | | |
| | HVLRYQDTPGVKMIVVLGEIGGTEEYKICRGIKEGRLTKPIVCWCIGTCATMFSSEVQFGHAGACAN | | |
| | QASETAVAKNQALKEAGVFVPRSFDELGEIIQSVYEDLVANGVIVPAQEVPPPTVPMDYSWARELGL | | |
| | IRKPASFMTSICDERGQELIYAGMPITEVFKEEMGIGGVLGLLWFQKRLPKYSCQFIEMCLMVTADH | | |
| | GPAVSGAHNTIICARAGKDLVSSLTSGLLTIGDRFGGALDAAAKMFSKAFDSGIIPMEFVNKMKKEG | | |
| | KLIMGIGHRVKSINNPDMRVQILKDYVRQHFPATPLLDYALEVEKITTSKKPNLILNVDGLIGVAFV | | |
| | DMLRNCGSFTREEADEYIDIGALNGIFVLGRSMGFIGHYLDQKRLKQGLYRHPWDDISYVLPEHMSM | | |
| | SEQ ID NO:91 | 4427 bp | |
| NOV6e, | GGCACGAGGCCGGGACAAAAGCCGGATCCCGGGAAGCTACCGGCTGCTGGGGTGCTCCGGATTTTGCG | | |
| CG142427-01 | | | |
| DNA Sequence | GGGTTCGTCGGGCCTGTGGAAGAAGCGCCGCGCACGGACTTCGGCAGAGGTAGAGCAGGTCTCTCTGC | | |
| | AGCCATGTCGGCCAAGGCAATTTCAGAGCAGACGGGCAAAGAACTCCTTTACAAGTTCATCTGTACCA | | |
| | CCTCAGCCATCCAGAATCGGTTCAAGTATGCTCGGGTCACTCCTGACACAGACTGGGCCCGCTTGCTG | | |
| | CAGGACCACCCCTGGCTGCTCAGCCAGAACTTGGTAGTCAAGCCAGACCAGCTGATCAAACGTCGTGG | | |
| | AAAACTTGGTCTCGTTGGGGTCAACCTCACTCTGGATGGGGTCAAGTCCTGGCTGAAGCCACGGCTGG | | |
| | GACAGGAAGCCACAGTTGGCAAGGCCACAGGCTTCCTCAAGAACTTTCTGATCGAGCCCTTCGTCCCC | | |
| | CACAGTCAGGCTGAGGAGTTCTATGTCTGCATCTATGCCACCCGAGAAGGGGACTACGTCCTGTTCCA | | |
| | CCACGAGGGGGGTGTGGACGTGGGTGATGTGGACGCCAAGGCCCAGAAGCTGCTTGTTGGCGTGGATG | | |
| | AGAAACTGAATCCTGAGGACATCAAAAAACACCTGTTGGTCCACGCCCCTGAAGACAAGAAAGAAATT | | |
| | CTGGCCAGTTTTATCTCCGGCCTCTTCAATTTCTACGAGGACTTGTACTTCACCTACCTCGAGATCAA | | |
| | TCCCCTTGTAGTGACCAAAGATGGAGTCTATGTCCTTGACTTGGCGGCCAAGGTGGACGCCACTGCCG | | |
| | ACTACATCTGCAAAGTGAAGTGGGGTGACATCGAGTTCCCTCCCCCCTTCGGGCGGGAGGCATATCCA | | |
| | GAGGAAGCCTACATTGCAGACCTCGATGCCAAAAGTGGGGCAAGCCTGAAGCTGACCTTGCTGAACCC | | |
| | CAAAGGGAGGATCTGGACCATGGTGGCCGGGGGTGGCGCCTCTGTCGTGTACAGCGATACCATCTGTG | | |
| | ATCTAGGGGGTGTCAACGAGCTGGCAAACTATGGGGAGTACTCAGGCGCCCCCAGCGAGCAGCAGACC | | |
| | TATGACTATGCCAAGACTATCCTCTCCCTCATGACCCGAGAGAAGCACCCAGATGGCAAGATCCTCAT | | |
| | CATTGGAGGCAGCATCGCAAACTTCACCAACGTGGCTGCCACGTTCAAGGGCATCGTGAGAGCAATTC | | |
| | GAGATTACCAGGGCCCCCTGAAGGAGCACGAAGTCACAATCTTTGTCCGAAGAGGTGGCCCCAACTAT | | |
| | CAGGAGGGCTTACGGGTGATGGGAGAAGTCGGGAAGACCACTGGGATCCCCATCCATGTCTTTGGCAC | | |
| | AGAGACTCACATGACGGCCATTGTGGGCATGGCCCTGGGCCACCGGCCCATCCCCAACCAGCCACCCA | | |
| | CAGCGGCCCACACTGCAAACTTCCTCCTCAACGCCAGCGGGAGCACATCGACGCCAGCCCCCAGCAGG | | |
| | ACAGCATCTTTTTCTGAGTCCAGGGCCGATGAGGTGGCGCCTGCAAAGAAGGCCAAGCCTGCCATGCC | | |
| | ACAAGATTCAGTCCCAAGTCCAAGATCCCTGCAAGGAAAGAGCACCACCCTCTTCAGCCGCCACACCA | | |
| | AGGCCATTGTGTGGGGCATGCAGACCCGGGCCGTGCAAGGCATGCTGGACTTTGACTATGTCTGCTCC | | |
| | CGAGACGAGCCCTCAGTGGCTGCCATGGTCTACCCTTTCACTGGGGACCACAAGCAGAAGTTTTACTG | | |
| | GGGGCACAAAGAGATCCTGATCCCTGTCTTCAAGAACATGGCTGATGCCATGAGGAAGCATCCGGAGG | | |
| | TAGATGTGCTCATCAACTTTGCCTCTCTCCGCTCTGCCTATGACAGCACCATGGAGACCATGAACTAT | | |
| | GCCCAGATCCGGACCATCGCCATCATAGCTGAAGGCATCCCTGAGGCCCTCACGAGAAAGCTGATCAA | | |
| | GAAGGCGGACCAGAAGGGAGTGACCATCATCGGACCTGCCACTGTTGGAGGCATCAAGCCTGGGTGCT | | |
| | TTAAGATTGGCAACACAGGTGGGATGCTGGACAACATCCTGGCCTCCAAACTGTACCGCCCAGGCAGC | | |
| | GTGGCCTATGTCTCACGTTCCGGAGGCATGTCCAACGAGCTCAACAATATCATCTCTCGGACCACGGA | | |
| | TGGCGTCTATGAGGGCGTGGCCATTGGTGGGGACAGGTACCCGGGCTCCACATTCATGGATCATGTGT | | |
| | TACGCTATCAGGACACTCCAGGAGTCAAAATGATTGTGGTTCTTGGAGAGATTGGGGGCACTGAGGAA | | |
| | TATAAGATTTGCCGGGGCATCAAGGAGGGCCGCCTCACTAAGCCCATCGTCTGCTGGTGCATCGGGAC | | |
| | GTGTGCCACCATGTTCTCCTCTGAGGTCCAGTTTGGCCATGCTGGAGCTTGTGCCAACCAGGCTTCTG | | |
| | AAACTGCAGTAGCCAAGAACCAGGCTTTGAAGGAAGCAGGAGTGTTTGTGCCCCGGAGCTTTGATGAG | | |
| | CTTGGAGAGATCATCCAGTCTGTATACGAAGATCTCGTGGCCAATGGAGTCATTGTACCTGCCCAGGA | | |
| | GGTGCCGCCCCCAACCGTGCCCATGGACTACTCCTGGGCCAGGGAGCTTGGTTTGATCCGCAAACCTG | | |
| | CCTCGTTCATGACCAGCATCTGCGATGAGCGAGGACAGGAGCTCATCTACGCGGGCATGCCCATCACT | | |
| | GAGGTCTTCAAGGAAGAGATGGGCATTGGCGGGGTCCTCGGCCTCCTCTGGTTCCAGAAAAGGTTGCC | | |
| | TAAGTACTCTTGCCAGTTCATTGAGATGTGTCTGATGGTGACAGCTGATCACGGGCCAGCCGTCTCTG | | |
| | GAGCCCACAACACCATCATTTGTGCGCGAGCTGGGAAAGACCTGGTCTCCAGCCTCACCTCGGGGCTG | | |
| | CTCACCATCGGGGATCGGTTTGGGGGTGCCTTGGATGCAGCAGCCAAGATGTTCAGTAAAGCCTTTGA | | |
| | CAGTGGCATTATCCCCATGGAGTTTGTGAACAAGATGAAGAAGGAAGGGAAGCTGATCATGGGCATTG | | |
| | GTCACCGAGTGAAGTCGATAAACAACCCAGACATGCGAGTGCAGATCCTCAAAGATTACGTCAGGCAG | | |
| | CACTTCCCTGCCACTCCTCTGCTCGATTATGCACTGGAAGTAGAGAAGATTACCACCTCGAAGAAGCC | | |
| | AAATCTTATCCTGAATGTAGATGGTCTCATCGGAGTCGCATTTGTAGACATGCTTAGAAACTGTGGGT | | |
| | CCTTTACTCGGGAGGAAGCTGATGAATATATTGACATTGGAGCCCTCAATGGCATCTTTGTGCTGGGA | | |
| | AGGAGTATGGGGTTCATTGGACACTATCTTGATCAGAAGAGGCTGAAGCAGGGGCTGTATCGTCATCC | | |
| | GTGGGATGATATTTCATATGTTCTTCCGGAACACATGAGCATGTAACAGAGCCAGGAACCCTACTGCA | | |
| | GTAAACTGAAGACAAGATCTCTTCCCCCAAGAAAAAGTGTACAGACAGCTGGCAGTGGAGCCTGCTTT | | |
| | ATTTAGCAGGGGCCTGGAATGTAAACAGCCACTGGGGTACAGGCACCGAAGACCAACATCCACAGGCT | | |
| | AACACCCCTTCAGTCCACACAAAGAAGCTTCATATTTTTTTTATAAGCATAGAAATAAAAACCAAGCC | | |
| | AATATTTGTGACTTTGCTCTGCTACCTGCTGTATTTATTATATGGAAGCATCTAAGTACTGTCAGGAT | | |
| | GGGGTCTTCCTCATTGTAGGGCGTTAGGATGTTGCTTTCTTTTTCCATTAGTTAAACATTTTTTTCTC | | |
| | CTTTGGAGGAAGGGAATGAAACATTTATGGCCTCAAGATACTATACATTTAAAGCACCCCAATGTCTC | | |
| | TCTTTTTTTTTTTTTACTTCCCTTTCTTCTTCCTTATATAACATGAAGAACATTGTATTAATCTGATT | | |
| | TTTAAAGATCTTTTTGTATGTTACGTGTTAAGGGCTTGTTTGGTATCCCACTGAAATGTTCTGTGTTG | | |
| | CAGACCAGAGTCTGTTTATGTCAGGGGGATGGGGCCATTGCATCCTTAGCCATTGTCACAAAATATGT | | |
| | GGAGTAGTAACTTAATATGTAAAGTTGTAACATACATACATTTAAAATGGAAATGCAGAAAGCTGTGA | | |
| | AATGTCTTGTGTCTTATGTTCTCTGTATTTATGCAGCTGATTTGTCTGTCTGTAACTGAAGTGTGGGT | | |
| | CCAAGGACTCCTAACTACTTTGCATCTGTAATCCACAAAGATTCTGGGCAGCTGCCACCTCAGTCTCT | | |
| | TCTCTGTATTATCATAGTCTGGTTTAAATAAACTATATAGTAACAAAAAAAAAAAAAAAAAAAAAAAA | | |
| | AAAAAAAAAAAAAAAAAAAAAAAAAAAAAAAAAAAAAAAAAAAAAAAAAAAAAAAAAAAAAAAAAAAA | | |
| | AAAAAAA | | |
| | ORF Start: ATG at 141 | | ORF Stop: TAA at 3444 |
| | SEQ ID NO:92 | 1101 aa | MW at 120838.0 kD |

| | |
|---|---|
| NOV6e, | MSAKAISEQTGKELLYKFICTTSAIQNRFKYARVTPDTDWARLLQDHPWLLSQNLVVKPDQLIKRRGK |
| CG142427-01 | |
| Protein | LGLVGVNLTLDGVKSWLKPRLGQEATVGKATGFLKNFLIEPFVPHSQAEEFYVCIYATREGDYVLFHH |
| Sequence | |
| | EGGVDVGDVDAKAQKLLVGVDEKLNPEDIKKHLLVHAPEDKKEILASFISGLFNFYEDLYFTYLEINP |
| | LVVTKDGVYVLDLAAKVDATADYICKVKWGDIEFPPPFGREAYPEEAYIADLDAKSGASLKLTLLNPK |
| | GRIWTMVAGGGASVVYSDTICDLGGVNELANYGEYSGAPSEQQTYDYAKTILSLMTREKHPDGKILII |
| | GGSIANFTNVAATFKGIVRAIRDYQGPLKEHEVTIFVRRGGPNYQEGLRVMGEVGKTTGIPIHVFGTE |
| | THMTAIVGMALGHRPIPNQPPTAAHTANFLLNASGSTSTPAPSRTASFSESRADEVAPAKKAKPAMPQ |
| | DSVPSPRSLQGKSTTLFSRHTKAIVWGMQTRAVQGMLDFDYVCSRDEPSVAAMVYPFTGDHKQKFYWG |
| | HKEILIPVFKNMADAMRKHPEVDVLINFASLRSAYDSTMETMNYAQIRTIAIIAEGIPEALTRKLIKK |
| | ADQKGVTIIGPATVGGIKPGCFKIGNTGGMLDNILASKLYRPGSVAYVSRSGGMSNELNNIISRTTDG |
| | VYEGVAIGGDRYPGSTFMDHVLRYQDTPGVKMIVVLGEIGGTEEYKICRGIKEGRLTKPIVCWCIGTC |
| | ATMFSSEVQFGHAGACANQASETAVAKNQALKEAGVFVPRSFDELGEIIQSVYEDLVANGVIVPAQEV |
| | PPPTVPMDYSWARELGLIRKPASFMTSICDERGQELIYAGMPITEVFKEEMGIGGVLGLLWFQKRLPK |
| | YSCQFIEMCLMVTADHGPAVSGAHNTIICARAGKDLVSSLTSGLLTIGDRFGGALDAAAKMFSKAFDS |
| | GIIPMEFVNKMKKEGKLIMGIGHRVRSINNPDMRVQILKDYVRQHFPATPLLDYALEVEKITTSKKPN |
| | LILNVDGLIGVAFVDMLRNCGSFTREEADEYIDIGALNGIFVLGRSMGFIGHYLDQKRLKQGLYRHPW |
| | DDISYVLPEHMSM |

Sequence comparison of the above protein sequences yields the following sequence relationships shown in Table 6B.

TABLE 6B
Comparison of NOV6a against NOV6b through NOV6e.

| Protein | NOV6a Residues/ | Identities/Similarities for |
|---|---|---|
| Sequence | Match Residues | the Matched Region |
| NOV6b | 1 . . . 1101 | 1091/1101 (99%) |
| | 5 . . . 1095 | 1091/1101 (99%) |
| NOV6c | 1 . . . 589 | 570/610 (93%) |
| | 5 . . . 604 | 573/610 (93%) |
| NOV6d | 1 . . . 1101 | 1065/1101 (96%) |
| | 5 . . . 1072 | 1065/1101 (96%) |
| NOV6e | 1 . . . 1101 | 1101/1101 (100%) |
| | 1 . . . 1101 | 1101/1101 (100%) |

Further analysis of the NOV6a protein yielded the following properties shown in Table 6C.

TABLE 6C
Protein Sequence Properties NOV6a

| | |
|---|---|
| SignalP analysis: | No Known Signal Sequence Predicted |
| PSORT II | PSG: a new signal peptide prediction method |
| analysis | N-region: length 8; pos.chg 1; neg.chg 1 |
| | H-region: length 3; peak value −7.32 |
| | PSG score: −11.72 |
| | GvH: von Heijne's method for signal seq. recognition |
| | GvH score (threshold: −2.1): −5.32 |
| | possible cleavage site: between 52 and 53 |
| | >>> Seems to have no N-terminal signal peptide |
| | ALOM: Klein et al's method for TM region allocation |
| | Init position for calculation: 1 |
| | Tentative number of TMS(s) for the threshold 0.5: 1 |
| | Number of TMS(s) for threshold 0.5: 1 |
| | INTEGRAL Likelihood = −2.71 Transmembrane 1021 -1037 |
| | PERIPHERAL Likelihood = 1.43 (at 1054) |
| | ALOM score: −2.71 (number of TMSs: 1) |
| | MTOP: Prediction of membrane topology (Hartmann et al.) |
| | Center position for calculation: 1028 |
| | Charge difference: −4.0 C(-2.0) - N( 2.0) |
| | N >= C: N-terminal side will be inside |
| | >>> Single TMS is located near the C-terminus |
| | >>> membrane topology: type Nt (cytoplasmic tail 1 to 1020) |
| | MITDISC: discrimination of mitochondrial targeting seq |

| | | | |
|---|---|---|---|
| R content: | 0 | Hyd Moment(75): | 10.90 |
| Hyd Moment(95): | 10.15 | G content: | 1 |
| D/E content: | 2 | SIT content: | 3 |
| Score: −5.48 | | | |

```
Gavel: prediction of cleavage sites for mitochondrial preseq
cleavage site motif not found
NUCDISC: discrimination of nuclear localization signals
pat4: none
pat7: PAKKAKP (4) at 466
bipartite: none
content of basic residues: 10.9%
NLS Score: −0.13
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-bindin motif: none
Actinin-type actin-binding motif:
type 1: none
type 2: none
NMYR: N-myristoylation pattern : none
Prenylation motif: none
memYQRL: transport motif from cell surface to Golgi: none
Tyrosines in the tail: too long tail
Dileucine motif in the tail: found
LL at 14
LL at 43
LL at 50
LL at 152
LL at 169
LL at 268
LL at 438
LL at 875
LL at 928
LL at 1003
checking 63 PROSITE DNA binding motifs:
Leucine zipper pattern (PS00029): *** found ***
LVVKPDQLIKRRGKLGLVGVNL at 55
none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
26.1 %: nuclear
21.7 %: mitochondrial
21.7 %: cytoplasmic
13.0 %: Golgi
  8.7 %: endoplasmic reticulum
  4.3 %: vesicles of secretory system
  4.3 %: peroxisomal
>> prediction for CG142427-05 is nuc (k = 23)
```

A search of the NOV6a protein against the Geneseq database, a proprietary database that contains sequences published in patents and patent publication, yielded several homologous proteins shown in Table 6D.

TABLE 6D
Geneseq Results for NOV6a

| | | | Identities/ | |
|---|---|---|---|---|
| Geneseq | Protein/Organism/Length | NOV6a Residues/ | Similarities for the | Expect |
| Identifier | [Patent #, Date] | Match Residues | Matched Region | Value |
| ABB61832 | Drosophila melanogaster | 1 . . . 1097 | 762/1099 (69%) | 0.0 |
| | polypeptide SEQ ID NO | 1 . . . 1083 | 895/1099 (81%) | |
| | 12288 - Drosophila | | | |
| | melanogaster, 1086 aa | | | |
| | [WO200171042-A2, | | | |
| | 27-SEP-2001] | | | |
| AAB56952 | Human prostate cancer | 753 . . . 1101 | 347/349 (99%) | 0.0 |
| | antigen protein sequence | 15 . . . 363 | 347/349 (99%) | |
| | SEQ ID NO:1530 - Homo | | | |
| | sapiens, 363 aa. | | | |
| | [WO200055174-A1, | | | |
| | 21-SEP-2000] | | | |
| AAY67408 | Arabidopsis ATP citrate | 492 . . . 1093 | 321/602 (53%) | 0.0 |
| | lyase (ACL) B-2 subunit - | 6 . . . 606 | 429/602 (70%) | |
| | Arabidopsis sp, 608 aa. | | | |
| | [WO200000619-A2, | | | |
| | 06-JAN-2000] | | | |
| AAG36247 | Arabidopsis thaliana protein | 492 . . . 1093 | 321/602 (53%) | 0.0 |
| | fragment SEQ ID NO: 44394 - | 6 . . . 606 | 429/602 (70%) | |
| | Arabidopsis thaliana, 681 | | | |
| | aa. [EP1033405-A2, | | | |
| | 06-SEP-2000] | | | |
| AAG36248 | Arabidopsis thaliana protein | 512 . . . 1093 | 313/582 (53%) | 0.0 |
| | fragment SEQ ID NO: 44395 - | 1 . . . 581 | 417/582 (70%) | |
| | Arabidopsis thaliana, 656 | | | |
| | aa. [EP1033405-A2, | | | |
| | 06-SEP-2000] | | | |

In a BLAST search of public sequence datbases, the NOV6a protein was found to have homology to the proteins shown in the BLASTP data in Table 6E.

TABLE 6E
Public BLASTP Results for NOV6a

| Protein | | | Identities/ | |
|---|---|---|---|---|
| Accession | | NOV6a Residues/ | Similarities for the | Expect |
| Number | Protein/Organism/Length | Match Residues | Matched Portion | Value |
| P53396 | ATP-citrate (pro-S-)-lyase | 1 . . . 1101 | 1100/1101 (99%) | 0.0 |
| | (EC 4.1.3.8) (Citrate | 1 . . . 1101 | 1101/1101 (99%) | |
| | cleavage enzyme) - Homo | | | |
| | sapiens (Human), 1101 aa. | | | |
| P16638 | ATP-citrate (pro-S-)-lyase | 1 . . . 1101 | 1074/1101 (97%) | 0.0 |
| | (EC 4.1.3.8) (Citrate | 1 . . . 1100 | 1086/1101 (98%) | |
| | cleavage enzyme) - Rattus | | | |
| | norvegicus (Rat), 1100 aa. | | | |
| Q91V92 | ATP-citrate (pro-S-)-lyase | 1 . . . 1101 | 1070/1101 (97%) | 0.0 |
| | (EC 4.1.3.8) (Citrate | 1 . . . 1091 | 1083/1101 (98%) | |
| | cleavage enzyme) - Mus | | | |
| | musculus (Mouse), 1091 aa. | | | |
| S21173 | ATP citrate (pro-S)-lyase - | 1 . . . 1101 | 1078/1106 (97%) | 0.0 |
| | human, 1105 aa. | 1 . . . 1105 | 1082/1106 (97%) | |
| Q8VIQ1 | ATP-citrate lyase - Rattus | 250 . . . 1101 | 835/852 (98%) | 0.0 |
| | norvegicus (Rat), 851 aa | 1 . . . 851 | 842/852 (98%) | |
| | (fragment). | | | |

PFam analysis predicts that the NOV6a protein contains the domains shown in the Table 6F.

TABLE 6F
Domain Analysis of NOV6a

| | | Identities/Similarities | |
|---|---|---|---|
| Pfam | | for the Matched | Expect |
| Domain | NOV6a Match Region | Region | Value |
| CoA_binding | 492 . . . 616 | 33/126 (26%) | 1.5e−19 |
| | | 88/126 (70%) | |
| ligase-CoA | 642 . . . 793 | 49/156 (31%) | 3.9e−53 |
| | | 126/156 (81%) | |

### Example 7

The NOV7 clone was analyzed, and the nucleotide and encoded polypeptide sequences are shown in Table 7A.

TABLE 7A
NOV7 Sequence Analysis

| | SEQ ID NO:93 | 1191 bp | |
|---|---|---|---|
| NOV7a, | AGTCCAGTGTGGTGGAATTCCACCATGAAGACCCTCATAGCCGCCTACTCCGGGGTCCTGCGCGGCGA | | |
| CG148010-03 | | | |
| DNA Sequence | GCGTCAGGCCGAGGCTGACCGGAGCCAGCGCTCTCACGGAGGACCTGCGCTGTCGCGCGAGGGGTCTG | | |
| | GGAGATGGGGCACTGGATCCAGCATCCTCTCCGCCCTCCAGGACCTCTTCTCTGTCACCTGGCTCAAT | | |
| | AGGTCCAAGGTGGAAAAGCAGCTACAGGTCATCTCAGTGCTCCAGTGGGTCCTGTCCTTCCTTGTACT | | |
| | GGGAGTGGCCTGCAGTGCCATCCTCATGTACATATTCTGCACTGATTGCTGGCTCATCGCTGTGCTCT | | |
| | ACTTCACTTGGCTGGTGTTTGACTGGAACACACCCAAGAAAGGTGGCAGGAGGTCACAGTGGGTCCGA | | |
| | AACTGGGCTGTGTGGCGCTACTTTCGAGACTACTTTCCCATCCAGCTGGTGAAGACACACAACCTGCT | | |
| | GACCACCAGGAACTATATCTTTGGATACCACCCCCATGGTATCATGGGCCTGGGTGCCTTCTGCAACT | | |
| | TCAGCACAGAGGCCACAGAAGTGAGCAAGAAGTTCCCAGGCATACGGCCTTACCTGGCTACACTGGCA | | |
| | GGCAACTTCCGAATGCCTGTGTTGAGGGAGTACCTGATGTCTGGAGGTATCTGCCCTGTCAGCCGGGA | | |
| | CACCATAGACTATTTGCTTTCAAAGAATGGGAGTGGCAATGCTATCATCATCGTGGTCGGGGGTGCGG | | |
| | CTGAGTCTCTGAGCTCCATGCCTGGCAAGAATGCAGTCACCCTGCGGAACCGCAAGGGCTTTGTGAAA | | |
| | CTGGCCCTGCGTCATGGAGCTGACCTGGTTCCCATCTACTCCTTTGGAGAGAATGAAGTGTACAAGCA | | |
| | GGTGATCTTCGAGGAGGGCTCCTGGGGCCGATGGGTCCAGAAGAAGTTCCAGAAATACATTGGTTTCG | | |
| | CCCCATGCATCTTCCATGGTCGAGGCCTCTTCTCCTCCGACACCTGGGGGCTGGTGCCCTACTCCAAG | | |
| | CCCATCACCACTGTTGTGGGAGAGCCCATCACCATCCCCAAGCTGGAGCACCCAACCCAGCAAGACAT | | |
| | CGACCTGTACCACACCATGTACATGGAGGCCCTGGTGAAGCTCTTCGACAAGCACAAGACCAAGTTCG | | |
| | GCCTCCCGGAGACTGAGGTCCTGGAGGTGAACTGA | | |
| | ORF Start: at 1 | | ORF Stop: TGA at 1189 |
| | SEQ ID NO:94 | 396 aa | MW at 44788.4 kD |
| NOV7a, | SPVWWNSTMKTLIAAYSGVLRGERQAEADRSQRSHGGPALSREGSGRWGTGSSILSALQDLFSVTWLN | | |
| CG148010-03 | | | |
| Protein | RSKVEKQLQVISVLQWVLSFLVLGVACSAILMYIFCTDCWLIAVLYFTWLVFDWNTPKKGGRRSQWVR | | |
| Sequence | | | |
| | NWAVWRYFRDYFPIQLVKTHNLLTTRNYIFGYHPHGIMGLGAFCNFSTEATEVSKKFPGIRPYLATLA | | |
| | GNFRMPVLREYLMSGGICPVSRDTIDYLLSKNGSGNAIIIVVGGAAESLSSMPGKNAVTLRNRKGFVK | | |
| | LALRHGADLVPIYSFGENEVYKQVIFEEGSWGRWVQKKFQKYIGFAPCIFHGRGLFSSDTWGLVPYSK | | |
| | PITTVVGEPITIPKLEHPTQQDIDLYHTMYMEALVKLFDKHKTKFGLPETEVLEVN | | |
| | SEQ ID NO:95 | 1230 bp | |
| NOV7b, | TTCAGCCATGAAGACCCTCATAGCCGCCTACTCCGGGGTCCTGCGCGGCGAGCGTCAGGCCGAGGCT | | |
| CG148010-01 | | | |
| DNA Sequence | GACCGGAGCCAGCGCTCTCACGGAGGACCCGTGTCGCGCGAGGGGTCTGGGAGATGGGGCACTGGAT | | |
| | CCAGCATCCTCTCCGCCCTCCAGGACCTCTTCTCTGTCACCTGGCTCAATAGGTCCAAGGTGGAAAA | | |
| | GCAGCTACAGGTCATCTCAGTGCTCCAGTGGGTCCTGTCCTTCCTTGTACTGGGAGTGGCCTGCAGT | | |
| | GCCATCCTCATGTACATATTCTGCACTGATTGCTGGCTCATCGCTGTGCTCTACTTCACTTGGCTGG | | |
| | TGTTTGACTGGAACACACCCAAGAAAGGTGGCAGGAGGTCACAGTGGGTCCGAAACTGGGCTGTGTG | | |
| | GCGCTACTTTCGAGACTACTTTCCCATCCAGCTGGTGAAGACACACAACCTGCTGACCACCAGGAAC | | |
| | TATATCTTTGGATACCACCCCCATGGTATCATGGGCCTGGGTGCCTTCTGCAACTTCAGCACAGAGG | | |
| | CCACAGAAGTGAGCAAGAAGTTCCCAGGCATACGGCCTTACCTGGCTACACTGGCAGGCAACTTCCG | | |
| | AATGCCTGTGTTGAGGGAGTACCTGATGTCTGGAGGTATCTGCCCTGTCAGCCGGGACACCATAGAC | | |
| | TATTTGCTTTCAAAGAATGGGAGTGGCAATGCTATCATCATCGTGGTCGGGGGTGCGGCTGAGTCTC | | |
| | TGAGCTCCATGCCTGGCAAGAATGCAGTCACCCTGCGGAACCGCAAGGGCTTTGTGAAACTGGCCCT | | |
| | GCGTCATGGAGCTGACCTGGTTCCCATCTACTCCTTTGGAGAGAATGAAGTGTACAAGCAGGTGATC | | |
| | TTCGAGGAGGGCTCCTGGGGCCGATGGGTCCAGAAGAAGTTCCAGAAATACATTGGTTTCGCCCCAT | | |
| | GCATCTTCCATGGTCGAGGCCTCTTCTCCTCCGACACCTGGGGGCTGGTGCCCTACTCCAAGCCCAT | | |
| | CACCACTGTTGTGGGAGAGCCCATCACCATCCCCAAGCTGGAGCACCCAACCCAGCAAGACATCGAC | | |
| | CTGTACCACACCATGTACATGGAGGCCCTGGTGAAGCTCTTCGACAAGCACAAGACCAAGTTCGGCC | | |
| | TCCCGGAGACTGAGGTCCTGGAGGTGAACTGAGCCAGCCTTCGGGGCCAATTCCCTGGAGGAACCAG | | |
| | CTGCAAATCACTTTTTTGCTCTGT | | |
| | ORF Start: ATG at 8 | | ORF Stop: TGA at 1169 |
| | SEQ ID NO:96 | 387 aa | MW at 43745.3 kD |
| NOV7b, | MKTLIAAYSGVLRGERQAEADRSQRSHGGPVSREGSGRWGTGSSILSALQDLFSVTWLNRSKVEKQL | | |
| CG148010-01 | | | |
| Protein | QVISVLQWVLSFLVLGVACSAILMYIFCTDCWLIAVLYFTWLVFDWNTPKKGGRRSQWVRNWAVWRY | | |
| Sequence | | | |
| | FRDYFPIQLVKTHNLLTTRNYIFGYHPHGIMGLGAFCNFSTEATEVSKKFPGIRPYLATLAGNFRMP | | |
| | VLREYLMSGGICPVSRDTIDYLLSKNGSGNAIIIVVGGAAESLSSMPGKNAVTLRNRKGFVKLALRH | | |
| | GADLVPIYSFGENEVYKQVIFEEGSWGRWVQKKFQKYIGFAPCIFHGRGLFSSDTWGLVPYSKPITT | | |
| | VVGEPITIPKLEHPTQQDIDLYHTMYMEALVKLFDKHKTKFGLPETEVLEVN | | |
| | SEQ ID NO:97 | 1191 bp | |
| NOV7c, | AGTCCAGTGTGGTGGAATTCCACCATGAAGACCCTCATAGCCGCCTACTCCGGGGTCCTGCGCGGCGA | | |
| 246864114 | | | |
| DNA Sequence | GCGTCAGGCCGAGGCTGACCGGAGCCAGCGCTCTCACGGAGGACCTGCGCTGTCGCGCGAGGGGTCTG | | |
| | GGAGATGGGGCACTGGATCCAGCATCCTCTCCGCCCTCCAGGACCTCTTCTCTGTCACCTGGCTCAAT | | |
| | AGGTCCAAGGTGGAAAAGCAGCTACAGGTCATCTCAGTGCTCCAGTGGGTCCTGTCCTTCCTTGTACT | | |
| | GGGAGTGGCCTGCAGTGCCATCCTCATGTACATATTCTGCACTGATTGCTGGCTCATCGCTGTGCTCT | | |
| | ACTTCACTTGGCTGGTGTTTGACTGGAACACACCCAAGAAAGGTGGCAGGAGGTCACAGTGGGTCCGA | | |
| | AACTGGGCTGTGTGGCGCTACTTTCGAGACTACTTTCCCATCCAGCTGGTGAAGACACACAACCTGCT | | |
| | GACCACCAGGAACTATATCTTTGGATACCACCCCCATGGTATCATGGGCCTGGGTGCCTTCTGCAACT | | |
| | TCAGCACAGAGGCCACAGAAGTGAGCAAGAAGTTCCCAGGCATACGGCCTTACCTGGCTACACTGGCA | | |
| | GGCAACTTCCGAATGCCTGTGTTGAGGGAGTACCTGATGTCTGGAGGTATCTGCCCTGTCAGCCGGGA | | |
| | CACCATAGACTATTTGCTTTCAAAGAATGGGAGTGGCAATGCTATCATCATCGTGGTCGGGGGTGCGG | | |
| | CTGAGTCTCTGAGCTCCATGCCTGGCAAGAATGCAGTCACCCTGCGGAACCGCAAGGGCTTTGTGAAA | | |
| | CTGGCCCTGCGTCATGGAGCTGACCTGGTTCCCATCTACTCCTTTGGAGAGAATGAAGTGTACAAGCA | | |
| | GGTGATCTTCGAGGAGGGCTCCTGGGGCCGATGGGTCCAGAAGAAGTTCCAGAAATACATTGGTTTCG | | |
| | CCCCATGCATCTTCCATGGTCGAGGCCTCTTCTCCTCCGACACCTGGGGGCTGGTGCCCTACTCCAAG | | |
| | CCCATCACCACTGTTGTGGGAGAGCCCATCACCATCCCCAAGCTGGAGCACCCAACCCAGCAAGACAT | | |
| | CGACCTGTACCACACCATGTACATGGAGGCCCTGGTGAAGCTCTTCGACAAGCACAAGACCAAGTTCG | | |
| | GCCTCCCGGAGACTGAGGTCCTGGAGGTGAACTGA | | |
| | ORF Start: at 1 | | ORF Stop: TGA at 1189 |
| | SEQ ID NO:98 | 396 aa | MW at 44788.4 kD |
| NOV7c, | SPVWWNSTMKTLIAAYSGVLRGERQAEADRSQRSHGGPALSREGSGRWGTGSSILSALQDLFSVTWLN | | |
| 246864114 | | | |
| Protein | RSKVEKQLQVISVLQWVLSFLVLGVACSAILMYIFCTDCWLIAVLYFTWLVFDWNTPKKGGRRSQWVR | | |
| Sequence | | | |
| | NWAVWRYFRDYFPIQLVKTHNLLTTRNYIFGYHPHGIMGLGAFCNFSTEATEVSKKFPGIRPYLATLA | | |
| | GNFRMPVLREYLMSGGICPVSRDTIDYLLSKNGSGNAIIIVVGGAAESLSSMPGKNAVTLRNRKGFVK | | |
| | LALRHGADLVPIYSFGENEVYKQVIFEEGSWGRWVQKKFQKYIGFAPCIFHGRGLFSSDTWGLVPYSK | | |
| | PITTVVGEPITIPKLEHPTQQDIDLYHTMYMEALVKLFDKHKTKFGLPETEVLEVN | | |
| | SEQ ID NO:99 | 1207 bp | |
| NOV7d, | CCAAGATCTACCATGAAGACCCTCATAGCCGCCTACTCCGGGGTCCTGCGCGGCGAGCGTCAGGCCG | | |
| 257448695 | | | |
| DNA Sequence | AGGCTGACCGGAGCCAGCGCTCTCACGGAGGACCTGCGCTGTCGCGCGAGGGGTCTGGGAGATGGGG | | |
| | CACTGGATCCAGCATCCTCTCCGCCCTCCAGGACCTCTTCTCTGTCACCTGGCTCAATAGGTCCAAG | | |
| | GTGGAAAAGCAGCTACAGGTCATCTCAGTGCTCCAGTGGGTCCTGTCCTTCCTTGTACTGGGAGTGG | | |
| | CCTGCAGTGCCATCCTCATGTACATATTCTGCACTGATTGCTGGCTCATCGCTGTGCTCTACTTCAC | | |
| | TTGGCTGGTGTTTGACTGGAACACACCCAAGAAAGGTGGCAGGAGGTCACAGTGGGTCCGAAACTGG | | |
| | GCTGTGTGGCGCTACTTTCGAGACTACTTTCCCATCCAGCTGGTGAAGACACACAACCTGCTGACCA | | |
| | CCAGGAACTATATCTTTGGATACCACCCCCATGGTATCATGGGCCTGGGTGCCTTCTGCAACTTCAG | | |
| | CACAGAGGCCACAGAAGTGAGCAAGAAGTTCCCAGGCATACGGCCTTACCTGGCTACACTGGCAGGC | | |
| | AACTTCCGAATGCCTGTGTTGAGGGAGTACCTGATGTCTGGAGGTATCTGCCCTGTCAGCCGGGACA | | |
| | CCATAGACTATTTGCTTTCAAAGAATGGGAGTGGCAATGCTATCATCATCGTGGTCGGGGGTGCGGC | | |
| | TGAGTCTCTGAGCTCCATGCCTGGCAAGAATGCAGTCACCCTGCGGAACCGCAAGGGCTTTGTGAAA | | |
| | CTGGCCCTGCGTCATGGAGCTGACCTGGTTCCCATCTACTCCTTTGGAGAGAATGAAGTGTACAAGC | | |
| | AGGTGATCTTCGAGGAGGGCTCCTGGGGCCGATGGGTCCAGAAGAAGTTCCAGAAATACATTGGTTT | | |
| | CGCCCCATGCATCTTCCATGGTCGAGGCCTCTTCTCCTCCGACACCTGGGGGCTGGTGCCCTACTCC | | |
| | AAGCCCATCACCACTGTTGTGGGAGAGCCCATCACCATCCCCAAGCTGGAGCACCCAACCCAGCAAG | | |
| | ACATCGACCTGTACCACACCATGTACATGGAGGCCCTGGTGAAGCTCTTCGACAAGCACAAGACCAA | | |
| | GTTCGGCCTCCCGGAGACTGAGGTCCTGGAGGTGAACCACCATCACCACCATCACTGAGCGGCCGCC | | |
| | A | | |
| | ORF Start: at 1 | | ORF Stop: TGA at 1195 |
| | SEQ ID NO:100 | 398 aa | MW at 45094.7 kD |
| NOV7d, | PRSTMKTLIAAYSGVLRGERQAEADRSQRSHGGPALSREGSGRWGTGSSILSALQDLFSVTWLNRSK | | |
| 257448695 | | | |
| Protein | VEKQLQVISVLQWVLSFLVLGVACSAILMYIFCTDCWLIAVLYFTWLVFDWNTPKKGGRRSQWVRNW | | |
| Sequence | | | |
| | AVWRYFRDYFPIQLVKTHNLLTTRNYIFGYHPHGIMGLGAFCNFSTEATEVSKKFPGIRPYLATLAG | | |
| | NFRMPVLREYLMSGGICPVSRDTIDYLLSKNGSGNAIIIVVGGAAESLSSMPGKNAVTLRNRKGFVK | | |
| | LALRHGADLVPIYSFGENEVYKQVIFEEGSWGRWVQKKFQKYIGFAPCIFHGRGLFSSDTWGLVPYS | | |
| | KPITTVVGEPITIPKLEHPTQQDIDLYHTMYMEALVKLFDKHKTKFGLPETEVLEVNHHHHHH | | |
| | SEQ ID NO:101 | 1189 bp | |
| NOV7e, | TACCATGAAGACCCTCATAGCCGCCTACTCCGGGGTCCTGCGCGGCGAGCGTCAGGCCGAGGCTGACC | | |
| 259357675 | | | |
| DNA Sequence | GGAGCCAGCGCTCTCACGGAGGACCTGCGCTGTCGCGCGAGGGGTCTGGGAGATGGGGCACTGGATCC | | |
| | AGCATCCTCTCCGCCCTCCAGGACCTCTTCTCTGTCACCTGGCTCAATAGGTCCAAGGTGGAAAAGCA | | |
| | GCTACAGGTCATCTCAGTGCTCCAGTGGGTCCTGTCCTTCCTTGTACTGGGAGTGGCCTGCAGTGCCA | | |
| | TCCTCATGTACATATTCTGCACTGATTGCTGGCTCATCGCTGTGCTCTACTTCACTTGGCTGGTGTTT | | |
| | GACTGGAACACACCCAAGAAAGGTGGCAGGAGGTCACAGTGGGTCCGAAACTGGGCTGTGTGGCGCTA | | |
| | CTTTCGAGACTACTTTCCCATCCAGCTGGTGAAGACACACAACCTGCTGACCACCAGGAACTATATCT | | |
| | TTGGATACCACCCCCATGGTATCATGGGCCTGGGTGCCTTCTGCAACTTCAGCACAGAGGCCACAGAA | | |
| | GTGAGCAAGAAGTTCCCAGGCATACGGCCTTACCTGGCTACACTGGCAGGCAACTTCCGAATGCCTGT | | |
| | GTTGAGGGAGTACCTGATGTCTGGAGGTATCTGCCCTGTCAGCCGGGACACCATAGACTATTTGCTTT | | |
| | CAAAGAATGGGAGTGGCAATGCTATCATCATCGTGGTCGGGGGTGCGGCTGAGTCTCTGAGCTCCATG | | |
| | CCTGGCAAGAATGCAGTCACCCTGCGGAACCGCAAGGGCTTTGTGAAACTGGCCCTGCGTCATGGAGC | | |
| | TGACCTGGTTCCCATCTACTCCTTTGGAGAGAATGAAGTGTACAAGCAGGTGATCTTCGAGGAGGGCT | | |
| | CCTGGGGCCGATGGGTCCAGAAGAAGTTCCAGAAATACATTGGTTTCGCCCCATGCATCTTCCATGGT | | |
| | CGAGGCCTCTTCTCCTCCGACACCTGGGGGCTGGTGCCCTACTCCAAGCCCATCACCACTGTTGTGGG | | |
| | AGAGCCCATCACCATCCCCAAGCTGGAGCACCCAACCCAGCAAGACATCGACCTGTACCACACCATGT | | |
| | ACATGGAGGCCCTGGTGAAGCTCTTCGACAAGCACAAGACCAAGTTCGGCCTCCCGGAGACTGAGGTC | | |
| | CTGGAGGTGAACCACCATCACCACCATCACTGA | | |
| | ORF Start: at 2 | | ORF Stop: TGA at 1187 |
| | SEQ ID NO:102 | 395 aa | MW at 44754.4 kD |
| NOV7e, | TMKTLIAAYSGVLRGERQAEADRSQRSHGGPALSREGSGRWGTGSSILSALQDLFSVTWLNRSKVEKQ | | |
| 259357675 | | | |
| Protein | LQVISVLQWVLSFLVLGVACSAILMYIFCTDCWLIAVLYFTWLVFDWNTPKKGGRRSQWVRNWAVWRY | | |
| Sequence | | | |
| | FRDYFPIQLVKTHNLLTTRNYIFGYHPHGIMGLGAFCNFSTEATEVSKKFPGIRPYLATLAGNFRMPV | | |
| LREYLMSGGICPVSRDTIDYLLSKNGSGNAIIIVVGGAAESLSSMPGKNAVTLRNRKGFVKLALRHGA | | | |
| | DLVPIYSFGENEVYKQVIFEEGSWGRWVQKKFQKYIGFAPCIFHGRGLFSSDTWGLVPYSKPITTVVG | | |
| | EPITIPKLEHPTQQDIDLYHTMYMEALVKLFDKHKTKFGLPETEVLEVNHHHHHH | | |
| | SEQ ID NO:103 | 1172 bp | |
| NOV7f, | CCACCATGAAGACCCTCATAGCCGCCTACTCCGGGGTCCTGCGCGGCGAGCGTCAGGCCGAGGCTGAC | | |
| 254868590 | | | |
| DNA Sequence | CGGAGCCAGCGCTCTCACGGAGGACCTGCGCTGTCGCGCGAGGGGTCTGGGAGATGGGGCACTGGATC | | |
| | CAGCATCCTCTCCGCCCTCCAGGACCTCTTCTCTGTCACCTGGCTCAATAGGTCCAAGGTGGAAAAGC | | |
| | AGCTACAGGTCATCTCAGTGCTCCAGTGGGTCCTGTCCTTCCTTGTACTGGGAGTGGCCTGCAGTGCC | | |
| | ATCCTCATGTACATATTCTGCACTGATTGCTGGCTCATCGCTGTGCTCTACTTCACTTGGCTGGTGTT | | |
| | TGACTGGAACACACCCAAGAAAGGTGGCAGGAGGTCACAGTGGGTCCGAAACTGGGCTGTGTGGCGCT | | |
| | ACTTTCGAGACTACTTTCCCATCCAGCTGGTGAAGACACACAACCTGCTGACCACCAGGAACTATATC | | |
| | TTTGGATACCACCCCCATGGTATCATGGGCCTGGGTGCCTTCTGCAACTTCAGCACAGAGGCCACAGA | | |
| | AGTGAGCAAGAAGTTCCCAGGCATACGGCCTTACCTGGCTACACTGGCAGGCAACTTCCGAATGCCTG | | |
| | TGTTGAGGGAGTACCTGATGTCTGGAGGTATCTGCCCTGTCAGCCGGGACACCATAGACTATTTGCTT | | |
| | TCAAAGAATGGGAGTGGCAATGCTATCATCATCGTGGTCGGGGGTGCGGCTGAGTCTCTGAGCTCCAT | | |
| | GCCTGGCAAGAATGCAGTCACCCTGCGGAACCGCAAGGGCTTTGTGAAACTGGCCCTGCGTCATGGAG | | |
| | CTGACCTGGTTCCCATCTACTCCTTTGGAGAGAATGAAGTGTACAAGCAGGTGATCTTCGAGGAGGGC | | |
| | TCCTGGGGCCGATGGGTCCAGAAGAAGTTCCAGAAATACATTGGTTTCGCCCCATGCATCTTCCATGG | | |
| | TCGAGGCCTCTTCTCCTCCGACACCTGGGGGCTGGTGCCCTACTCCAAGCCCATCACCACTGTTGTGG | | |
| | GAGAGCCCATCACCATCCCCAAGCTGGAGCACCCAACCCAGCAAGACATCGACCTGTACCACACCATG | | |
| | TACATGGAGGCCCTGGTGAAGCTCTTCGACAAGCACAAGACCAAGTTCGGCCTCCCGGAGACTGAGGT | | |
| | CCTGGAGGTGAACTGA | | |
| | ORF Start: at 3 | | ORF Stop: TGA at 1170 |
| | SEQ ID NO:104 | 389 aa | MW at 43931.5 kD |
| NOV7f, | TMKTLIAAYSGVLRGERQAEADRSQRSHGGPALSREGSGRWGTGSSILSALQDLFSVTWLNRSKVEKQ | | |
| 254868590 | | | |
| Protein | LQVISVLQWVLSFLVLGVACSAILMYIFCTDCWLIAVLYFTWLVFDWNTPKKGGRRSQWVRNWAVWRY | | |
| Sequence | | | |
| | FRDYFPIQLVKTHNLLTTRNYIFGYHPHGIMGLGAFCNFSTEATEVSKKFPGIRPYLATLAGNFRMPV | | |
| | LREYLMSGGICPVSRDTIDYLLSKNGSGNAIIIVVGGAAESLSSMPGKNAVTLRNRKGFVKLALRHGA | | |
| | DLVPIYSFGENEVYKQVIFEEGSWGRWVQKKFQKYIGFAPCIFHGRGLFSSDTWGLVPYSKPITTVVG | | |
| | EPITIPKLEHPTQQDIDLYHTMYMEALVKLFDKHKTKFGLPETEVLEVN | | |
| | SEQ ID NO:105 | 1198 bp | |
| NOV7g, | CCAGAATTCCACCATGAAGACCCTCATAGCCGCCTACTCCGGGGTCCTGCGCGGCGAGCGTCAGGCC | | |
| CG148010-02 | | | |
| DNA Sequence | GAGGCTGACCGGAGCCAGCGCTCTCACGGAGGACCTGCGCTGTCGCGCGAGGGGTCTGGGAGATGGG | | |
| | GCACTGGATCCAGCATCCTCTCCGCCCTCCAGGACCTCTTCTCTGTCACCTGGCTCAATAGGTCCAA | | |
| | GGTGGAAAAGCAGCTACAGGTCATCTCAGTGCTCCAGTGGGTCCTGTCCTTCCTTGTACTGGGAGTG | | |
| | GCCTGCAGTGCCATCCTCATGTACATATTCTGCACTGATTGCTGGCTCATCGCTGTGCTCTACTTCA | | |
| | CTTGGCTGGTGTTTGACTGGAACACACCCAAGAAAGGTGGCAGGAGGTCACAGTGGGTCCGAAACTG | | |
| | GGCTGTGTGGCGCTACTTTCGAGACTACTTTCCCATCCAGCTGGTGAAGACACACAACCTGCTGACC | | |
| | ACCAGGAACTATATCTTTGGATACCACCCCCATGGTATCATGGGCCTGGGTGCCTTCTGCAACTTCA | | |
| | GCACAGAGGCCACAGAAGTGAGCAAGAAGTTCCCAGGCATACGGCCTTACCTGGCTACACTGGCAGG | | |
| | CAACTTCCGAATGCCTGTGTTGAGGGAGTACCTGATGTCTGGAGGTATCTGCCCTGTCAGCCGGGAC | | |
| | ACCATAGACTATTTGCTTTCAAAGAATGGGAGTGGCAATGCTATCATCATCGTGGTCGGGGGTGCGG | | |
| | CTGAGTCTCTGAGCTCCATGCCTGGCAAGAATGCAGTCACCCTGCGGAACCGCAAGGGCTTTGTGAA | | |
| | ACTGGCCCTGCGTCATGGAGCTGACCTGGTTCCCATCTACTCCTTTGGAGAGAATGAAGTGTACAAG | | |
| | CAGGTGATCTTCGAGGAGGGCTCCTGGGGCCGATGGGTCCAGAAGAAGTTCCAGAAATACATTGGTT | | |
| | TCGCCCCATGCATCTTCCATGGTCGAGGCCTCTTCTCCTCCGACACCTGGGGGCTGGTGCCCTACTC | | |
| | CAAGCCCATCACCACTGTTGTGGGAGAGCCCATCACCATCCCCAAGCTGGAGCACCCAACCCAGCAA | | |
| | GACATCGACCTGTACCACACCATGTACATGGAGGCCCTGGTGAAGCTCTTCGACAAGCACAAGACCA | | |
| | AGTTCGGCCTCCCGGAGACTGAGGTCCTGGAGGTGAACTGAGCGGCCGCTTTTTTCCTT | | |
| | ORF Start: ATG at 14 | | ORF Stop: at 1118 |
| | SEQ ID NO:106 | 368 aa | MW at 41503.7 kD |
| NOV7g, | MKTLIAAYSGVLRGERQAEADRSQRSHGGPALSREGSGRWGTGSSILSALQDLFSVTWLNRSKVEKQ | | |
| CG148010-02 | | | |
| Protein | LQVISVLQWVLSFLVLGVACSAILMYIFCTDCWLIAVLYFTWLVFDWNTPKKGGRRSQWVRNWAVWR | | |
| Sequence | | | |
| | YFRDYFPIQLVKTHNLLTTRNYIFGYHPHGIMGLGAFCNFSTEATEVSKKFPGIRPYLATLAGNFRM | | |
| | PVLREYLMSGGICPVSRDTIDYLLSKNGSGNAIIIVVGGAAESLSSMPGKNAVTLRNRKGFVKLALR | | |
| | HGADLVPIYSFGENEVYKQVIFEEGSWGRWVQKKFQKYIGFAPCIFHGRGLFSSDTWGLVPYSKPIT | | |
| | TVVGEPITIPKLEHPTQQDIDLYHTMYMEALVK | | |
| | SEQ ID NO:107 | 1189 bp | |
| NOV7h, | TACCATGAAGACCCTCATAGCCGCCTACTCCGGGGTCCTGCGCGGCGAGCGTCAGGCCGAGGCTGAC | | |
| CG148010-04 | | | |
| DNA Sequence | CGGAGCCAGCGCTCTCACGGAGGACCTGCGCTGTCGCGCGAGGGGTCTGGGAGATGGGGCACTGGAT | | |
| | CCAGCATCCTCTCCGCCCTCCAGGACCTCTTCTCTGTCACCTGGCTCAATAGGTCCAAGGTGGAAAA | | |
| | GCAGCTACAGGTCATCTCAGTGCTCCAGTGGGTCCTGTCCTTCCTTGTACTGGGAGTGGCCTGCAGT | | |
| | GCCATCCTCATGTACATATTCTGCACTGATTGCTGGCTCATCGCTGTGCTCTACTTCACTTGGCTGG | | |
| | TGTTTGACTGGAACACACCCAAGAAAGGTGGCAGGAGGTCACAGTGGGTCCGAAACTGGGCTGTGTG | | |
| | GCGCTACTTTCGAGACTACTTTCCCATCCAGCTGGTGAAGACACACAACCTGCTGACCACCAGGAAC | | |
| | TATATCTTTGGATACCACCCCCATGGTATCATGGGCCTGGGTGCCTTCTGCAACTTCAGCACAGAGG | | |
| | CCACAGAAGTGAGCAAGAAGTTCCCAGGCATACGGCCTTACCTGGCTACACTGGCAGGCAACTTCCG | | |
| | AATGCCTGTGTTGAGGGAGTACCTGATGTCTGGAGGTATCTGCCCTGTCAGCCGGGACACCATAGAC | | |
| | TATTTGCTTTCAAAGAATGGGAGTGGCAATGCTATCATCATCGTGGTCGGGGGTGCGGCTGAGTCTC | | |
| | TGAGCTCCATGCCTGGCAAGAATGCAGTCACCCTGCGGAACCGCAAGGGCTTTGTGAAACTGGCCCT | | |
| | GCGTCATGGAGCTGACCTGGTTCCCATCTACTCCTTTGGAGAGAATGAAGTGTACAAGCAGGTGATC | | |
| | TTCGAGGAGGGCTCCTGGGGCCGATGGGTCCAGAAGAAGTTCCAGAAATACATTGGTTTCGCCCCAT | | |
| | GCATCTTCCATGGTCGAGGCCTCTTCTCCTCCGACACCTGGGGGCTGGTGCCCTACTCCAAGCCCAT | | |
| | CACCACTGTTGTGGGAGAGCCCATCACCATCCCCAAGCTGGAGCACCCAACCCAGCAAGACATCGAC | | |
| | CTGTACCACACCATGTACATGGAGGCCCTGGTGAAGCTCTTCGACAAGCACAAGACCAAGTTCGGCC | | |
| | TCCCGGAGACTGAGGTCCTGGAGGTGAACCACCATCACCACCATCACTGA | | |
| | ORF Start: at 2 | | ORF Stop: TGA at 1187 |
| | SEQ ID NO:108 | 395 aa | MW at 44754.4 kD |

| | |
|---|---|
| NOV7h, | TMKTLIAAYSGVLRGERQAEADRSQRSHGGPALSREGSGRWGTGSSILSALQDLFSVTWLNRSKVEK |
| CG148010-04 | |
| Protein | QLQVISVLQWVLSFLVLGVACSAILMYIFCTDCWLIAVLYFTWLVFDWNTPKKGGRRSQWVRNWAVW |
| Sequence | |
| | RYFRDYFPIQLVKTHNLLTTRNYIFGYHPHGIMGLGAFCNFSTEATEVSKKFPGIRPYLATLAGNFR |
| MPVLREYLMSGGICPVSRDTIDYLLSKNGSGNAIIIVVGGAAESLSSMPGKNAVTLRNRKGFVKLAL | |
| | RHGADLVPIYSFGENEVYKQVIFEEGSWGRWVQKKFQKYIGFAPCIFHGRGLFSSDTWGLVPYSKPI |
| | TTVVGEPITIPKLEHPTQQDIDLYHTMYMEALVKLFDKHKTKFGLPETEVLEVNHHHHHH |

Sequence comparison of the above protein sequences yields the following sequence relationships shown in Table 7B.

TABLE 7B
Comparison of NOV7a against NOV7b through NOV7h.

| | NOV7a | Identities/ |
|---|---|---|
| | Residues/ | Similarities for |
| Protein | Match | the Matched |
| Sequence | Residues | Region |
| NOVTb | 9 . . . 396 | 386/388 (99%) |
| | 1 . . . 387 | 387/388 (99%) |
| NOV7c | 1 . . . 396 | 396/396 (100%) |
| | 1 . . . 396 | 396/396 (100%) |
| NOV7d | 7 . . . 396 | 390/390 (100%) |
| | 3 . . . 392 | 390/390 (100%) |
| NOV7e | 8 . . . 396 | 389/389 (100%) |
| | 1 . . . 389 | 389/389 (100%) |
| NOV7f | 8 . . . 396 | 389/389 (100%) |
| | 1 . . . 389 | 389/389 (100%) |
| NOV7g | 9 . . . 376 | 368/368 (100%) |
| | 1 . . . 368 | 368/368 (100%) |
| NOV7h | 8 . . . 396 | 389/389 (100%) |
| | 1 . . . 389 | 389/389 (100%) |

Further analysis of the NOV7a protein yielded the following properties shown in Table 7C.

TABLE 7C
Protein Sequence Properties NOV7a

| SignalP | | | | |
|---|---|---|---|---|
| analysis: | No Known Signal Sequence Predicted | | | |
| PSORT II | PSG: a new signal peptide prediction method | | | |
| analysis: | N-region: length 10; pos.chg 1; neg.chg 0 | | | |
| | H-region: length 10; peak value 6.60 | | | |
| | PSG score: 2.20 | | | |
| | GvH: von Heijne's method for signal seq. recognition | | | |
| | GvH score (threshold: −2.1): −5.66 | | | |
| | possible cleavage site: between 22 and 23 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL Likelihood = −8.23 Transmembrane | | | |
| | 88-104 | | | |
| | PERIPHERAL Likelihood = 1.16 (at 241) | | | |
| | ALOM score: −8.23 (number of TMSs: 1) | | | |
| | MTOP: Prediction of membrane topology (Hartmann et al.) | | | |
| | Center position for calculation: 95 | | | |
| | Charge difference: −2.0 C(−1.0) −N(1.0) | | | |
| | N >= C: N-terminal side will be inside | | | |
| | >>> membrane topology: type 2 (cytoplasmic tail 1 to 88) | | | |
| | MITDISC: discrimination of mitochondrial targeting seq | | | |
| | R content: | 1 | Hyd Moment(75): | 4.28 |
| | Hyd Moment(95): | 4.11 | G content: | 2 |
| | D/E content: | 1 | S/T content: | 5 |

```
Score: −4.01
Gavel: prediction of cleavage sites for mitochondrial
preseq
R-2 motif at 31 LRG|ER
NUCDISC: discrimination of nuclear localization signals
pat4: none
pat7: none
bipartite: none
content of basic residues: 11.1%
NLS Score: −0.47
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
--------------------------
Final Results (k = 9/23):
34.8%: mitochondrial
26.1%: cytoplasmic
13.0%: Golgi
  8.7%: endoplasmic reticulum
  4.3%: extracellular, including cell wall
  4.3%: vacuolar
  4.3%: nuclear
  4.3%: vesicles of secretory system
>> prediction for CG148010-03 is mit (k = 23)
```

A search of the NOV7a protein against the Geneseq database, a proprietary database that contains sequences published in patents and patent publication, yielded several homologous proteins shown in Table 7D.

TABLE 7D
Geneseq Results for NOV7a

| | | NOV7a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB75677 | Breast protein-eukaryotic | 9 . . . 396 | 388/388 (100%) | 0.0 |
| | conserved gene 1 | 1 . . . 388 | 388/388 (100%) | |
| | (BSTP-ECG1) protein - | | | |
| | Homo sapiens, 388 aa. | | | |
| | [WO200208260-A2, | | | |
| | 31 JAN. 2002] | | | |
| AAB66170 | Protein of the invention #82 - | 9 . . . 396 | 388/388 (100%) | 0.0 |
| | Unidentified, 388 aa. | 1 . . . 388 | 388/388 (100%) | |
| | [WO200078961-A1, | | | |
| | 28 DEC. 2000] | | | |
| AAU29191 | Human PRO polypeptide | 9 . . . 396 | 388/388 (100%) | 0.0 |
| | sequence #168 - Homo | 1 . . . 388 | 388/388 (100%) | |
| | sapiens, 388 aa. | | | |
| | [WO200168848-A2, | | | |
| | 20 SEP. 2001] | | | |
| AAY99421 | Human PRO1433 (UNQ738) | 9 . . . 396 | 388/388 (100%) | 0.0 |
| | amino acid sequence SEQ ID | 1 . . . 388 | 388/388 (100%) | |
| | NO: 292 - Homo sapiens, 388 | | | |
| | aa. [WO200012708-A2, | | | |
| | 09 MAR. 2000] | | | |
| AAY48474 | Human breast | 209 . . . 396 | 188/188 (100%) | e−108 |
| | tumour-associated protein | 1 . . . 188 | 188/188 (100%) | |
| | 19 - Homo sapiens, 188 aa. | | | |
| | [DE19813835-A1, | | | |
| | 23 SEP. 1999] | | | |

In a BLAST search of public sequence datbases, the NOV7a protein was found to have homology to the proteins shown in the BLASTP data in Table 7E.

TABLE 7E
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NDB7 | Hypothetical protein - Homo | 7 . . . 396 | 389/390 (99%) | 0.0 |
| | sapiens (Human), 434 aa | 45 . . . 434 | 389/390 (99%) | |
| | (fragment). | | | |
| Q96PD7 | Diacylglycerol | 9 . . . 396 | 388/388 (100%) | 0.0 |
| | acyltransferase 2 | 1 . . . 388 | 388/388 (100%) | |
| | (Hypothetical protein) | | | |
| | (GS1999full protein) - Homo | | | |
| | sapiens (Human), 388 aa. | | | |
| Q9DCV3 | 0610010B06Rik protein | 9 . . . 396 | 369/388 (95%) | 0.0 |
| | (Diacylglycerol | 1 . . . 388 | 377/388 (97%) | |
| | acyltransferase 2) - Mus | | | |
| | musculus (Mouse), 388 aa. | | | |
| Q8TAB1 | BA351K23.5 (Novel protein) - | 105 . . . 395 | 152/291 (52%) | 1e−93 |
| | Homo sapiens (Human), 296 | 6 . . . 295 | 214/291 (73%) | |
| | aa (fragment). | | | |
| Q96PD6 | Diacylglycerol | 71 . . . 393 | 164/323 (50%) | 5e−92 |
| | acyltransferase 2-like | 11 . . . 331 | 222/323 (67%) | |
| | protein - Homo sapiens | | | |
| | (Human), 334 aa. | | | |

### Example 8

The NOV8 clone was analyzed, and the nucleotide and encoded polypeptide sequences are shown in Table 8A.

TABLE 8A
NOV8 Sequence Analysis

| | SEQ ID NO:109 | 2127 bp | |
|---|---|---|---|
| NOV8a, | CGCGGATCCACCATGCAAGCCCATGAGCTGTTCCGGTATTTTCGAATGCCAGAGCTGGTTGACTTCCG | | |
| CG148278-02 | | | |
| DNA Sequence | ACAGTACGTGCGTACTCTTCCGACCAACACGCTTATGGGCTTCGGAGCTTTTGCAGCACTCACCACCT | | |
| | TCTGGTACGCCACGAGACCCAAACCCCTGAAGCCGCCATGCGACCTCTCCATGCAGTCAGTGGAAGTG | | |
| | GCGGGTAGTGGTGGTGCACGAAGATCCGCACTACTTGACAGCGACGAGCCCTTGGTGTATTTCTATGA | | |
| | TGATGTCACAACATTATACGAAGGTTTCCAGAGGGGAATACAGGTGTCAAATAATGGCCCTTGTTTAG | | |
| | GCTCTCGGAAACCAGACCAACCCTACGAATGGCTTTCATATAAACAGGTTGCAGAATTGTCGGAGTGC | | |
| | ATAGGCTCAGCACTGATCCAGAAGGGCTTCAAGACTGCCCCAGATCAGTTCATTGGCATCTTTGCTCA | | |
| | AAATAGACCTGAGTGGGTGATTATTGAACAAGGATGCTTTGCTTATTCGATGGTGATCGTTCCACTTT | | |
| | ATGATACCCTTGGAAATGAAGCCATCACGTACATAGTCAACAAAGCTGAACTCTCTCTGGTTTTTGTT | | |
| | GACAAGCCAGAGAAGGCCAAACTCTTATTAGAGGGTGTAGAAAATAAGTTAATACCAGGCCTTAAAAT | | |
| | CATAGTTGTCATGGATGCCTACGGCAGTGAACTGGTGGAACGAGGCCAGAGGTGTGGGGTGGAAGTCA | | |
| | CCAGCATGAAGGCGATGGAGGACCTGGGAAGAGCCAACAGACGGAAGCCCAAGCCTCCAGCACCTGAA | | |
| | GATCTTGCAGTAATTTGTTTCACAAGTGGAACTACAGGCAACCCCAAAGGAGCAATGGTCACTCACCG | | |
| | AAACATAGTGAGCGATTGTTCAGCTTTTGTGAAAGCAACAGAGAATACAGTCAATCCTTGCCCAGATG | | |
| | ATACTTTGATATCTTTCTTGCCTCTCGCCCATATGTTTGAGAGAGTTGTAGAGTGTGTAATGCTGTGT | | |
| | CATGGAGCTAAAATCGGATTTTTCCAAGGAGATATCAGGCTGCTCATGGATGACCTCAAGGTGCTTCA | | |
| | ACCCACTGTCTTCCCCGTGGTTCCAAGACTGCTGAACCGGATGTTTGACCGAATTTTCGGACAAGCAA | | |
| | ACACCACGCTGAAGCGATGGCTCTTGGACTTTGCCTCCAAGAGGAAAGAAGCAGAGCTTCGCAGCGGC | | |
| | ATCATCAGAAACAACAGCCTGTGGGACCGGCTGATCTTCCACAAAGTACAGTCGAGCCTGGGCGGAAG | | |
| | AGTCCGGCTGATGGTGACAGGAGCCGCCCCGGTGTCTGCCACTGTGCTGACGTTCCTCAGAGCAGCCC | | |
| | TGGGCTGTCAGTTTTATGAAGGATACGGACAGACAGAGTGCACTGCCGGGTGCTGCCTGACCATGCCT | | |
| | GGAGACTGGACCGCAGGCCATGTTGGGGCCCCGATGCCGTGCAATTTGATAAAACTTGTTGATGTGGA | | |
| | AGAAATGAATTACATGGCTGCCGAGGGCGAGGGCGAGGTGTGTGTGAAAGGGCCAAATGTATTTCAGG | | |
| | GCTACTTGAAGGACCCAGCGAAAACAGCAGAAGCTTTGGACAAAGACGGCTGGTTACACACAGGGGAC | | |
| | ATTGGAAAATGGTTACCAAATGGCACCTTGAAAATTATCGACCGGAAAAAGCACATATTTAAGCTGGC | | |
| | ACAAGGAGAATACATAGCCCCTGAAAAGATTGAAAATATCTACATGCGAAGTGAGCCTGTTGCTCAGG | | |
| | TGTTTGTCCACGGAGAAAGCCTGCAGGCATTTCTCATTGCAATTGTGGTACCAGATGTTGAGACATTA | | |
| | TGTTCCTGGGCCCAAAAGAGAGGATTTGAAGGGTCGTTTGAGGAACTGTGCAGAAATAAGGATGTCAA | | |
| | AAAAGCTATCCTCGAAGATATGGTGAGACTTGGGAAGGATTCTGGTCTGAAACCATTTGAACAGGTCA | | |
| | AAGGCATCACATTGCACCCTGAATTATTTTCTATCGACAATGGCCTTCTGACTCCAACAATGAAGGCG | | |
| | AAAAGGCCAGAGCTGCGGAACTATTTCAGGTCGCAGATAGATGACCTCTATTCCATCATCAAGGTTTA | | |
| | GGCGGCCGCTTTTTTCCTT | | |
| | ORF Start: at 1 | | ORF Stop: TAG at 2107 |
| | SEQ ID NO:110 | 702 aa | MW at 78355.9 kD |
| NOV8a, | RGSTMQAHELFRYFRMPELVDFRQYVRTLPTNTLMGFGAFAALTTFWYATRPKPLKPPCDLSMQSVEV | | |
| CG148278-02 | | | |
| Protein | AGSGGARRSALLDSDEPLVYFYDDVTTLYEGFQRGIQVSNNGPCLGSRKPDQPYEWLSYKQVAELSEC | | |
| Sequence | | | |
| | IGSALIQKGFKTAPDQFIGIFAQNRPEWVIIEQGCFAYSMVIVPLYDTLGNEAITYIVNKAELSLVFV | | |
| | DKPEKAKLLLEGVENKLIPGLKIIVVMDAYGSELVERGQRCGVEVTSMKAMEDLGRANRRKPKPPAPE | | |
| | DLAVICFTSGTTGNPKGAMVTHRNIVSDCSAFVKATENTVNPCPDDTLISFLPLAHMFERVVECVMLC | | |
| | HGAKIGFFQGDIRLLMDDLKVLQPTVFPVVPRLLNRMFDRIFGQANTTLKRWLLDFASKRKEAELRSG | | |
| | IIRNNSLWDRLIFHKVQSSLGGRVRLMVTGAAPVSATVLTFLRAALGCQFYEGYGQTECTAGCCLTMP | | |
| | GDWTAGHVGAPMPCNLIKLVDVEEMNYMAAEGEGEVCVKGPNVFQGYLKDPAKTAEALDKDGWLHTGD | | |
| | IGKWLPNGTLKIIDRKKHIFKLAQGEYIAPEKIENIYMRSEPVAQVFVHGESLQAFLIAIVVPDVETL | | |
| | CSWAQKRGFEGSFEELCRNKDVKKAILEDMVRLGKDSGLKPFEQVKGITLHPELFSIDNGLLTPTMKA | | |
| | KRPELRNYFRSQIDDLYSIIKV | | |
| | SEQ ID NO:111 | 3188 bp | |
| NOV8b, | CGGGCAGTGACAGCCGGCGCGGATCGCGCGTCCACGGAGGAGAATCAGCTTAGAGAACTATCAACAC | | |
| CG148278-01 | | | |
| DNA Sequence | AGGACAATGCAAGCCCATGAGCTGTTCCGGTATTTTCGAATGCCAGAGCTGGTTGACTTCCGACAGT | | |
| | GCGTGACTCTTCCGACCAACACGCTTATGGGCTTCGGAGCTTTTTCCAGACGACTCACCACCTTCTG | | |
| | GCGGCCACGCCACCCAAAACCCCTGAAGCCGCCATGGCACCTCTCCATGCAGTCAGTGGAAGTGGCG | | |
| | GGTAGTGGTGGTGCACGAAGATCCGCACTACTTGACAGCGACGAGCCCTTGGTGTATTTCTATGATG | | |
| | ATGTTACAACATTATACGAAGGTTTCCAGAGAGGGATACAGGTGTCAAATAATGGCCCTTGTTTAGG | | |
| | CTCTCGGAAACCAGACCAACCCTATGAATGGCTTTCATATAAACAGGTTGCAGAATTGTCGGAGTGC | | |
| | ATAGGCTCAGCACTGATCCAGAAGGGCTTCAAGACTGCCCCAGATCAGTTCATTGGCATCTTTGCTC | | |
| | AAAATAGACCTGAGTGGGTGATTATTGAACAAGGATGCTTTGCTTATTCGATGGTGATCGTTCCACT | | |
| | TTATGATACCCTTGGAAATGAAGCCATCACGTACATAGTCAACAAAGCTGAACTCTCTCTGGTTTTT | | |
| | GTTGACAAGCCAGAGAAGGCCAAACTCTTATTAGAGGGTGTAGAAAATAAGTTAATACCAGGCCTTA | | |
| | AAATCATAGTTGTCATGGACTCGTACGGCAGTGAACTGGTGGAACGAGGCCAGAGGTGTGGGGTGGA | | |
| | AGTCACCAGCATGAAGGCGATGGAGGACCTGGGAAGAGCCAACAGACGGAAGCCCAAGCCTCCAGCA | | |
| | CCTGAAGATCTTGCAGTAATTTGTTTCACAAGTGGAACTACAGGCAACCCCAAAGGAGCAATGGTCA | | |
| | CTCACCGAAACATAGTGAGCGATTGTTCAGCTTTTGTGAAAGCAACAGAGAATACAGTCAATCCTTG | | |
| | CCCAGATGATACTTTGATATCTTTCTTGCCTCTCGCCCATATGTTTGAGAGAGTTGTAGAGTGTGTA | | |
| | ATGCTGTGTCATGGAGCTAAAATCGGATTTTTCCAAGGAGATATCAGGCTGCTCATGGATGACCTCA | | |
| | AGGTGCTTCAACCCACTGTCTTCCCCGTGGTTCCAAGACTGCTGAACCGGATGTTTGACCGAATTTT | | |
| | CGGACAAGCAAACACCACCGTGAAGCGATGGCTCTTGGACTTTGCCTCCAAGAGGAAAGAAGCAGAC | | |
| | GTTCGCAGCGGCATCATCAGAAACAACAGCCTGTGGGACCGGCTGATCTTCCACAAAGTACAGTCGA | | |
| | GCCTGGGCGGAAGAGTCCGGCTGATGGTGACAGGAGCCGCCCCGGTGTCTGCCACTGTGCTGACGTT | | |
| | CCTCAGAGCAGCCCTGGGCTGTCAGTTTTATGAAGGATACGGACAGACAGAGTGCACTGCCGGGTGC | | |
| | TGCCTAACCATGCCTGGAGACTGGACCACAGGCCATGTTGGGGCCCCGATGCCGTGCAATTTGATAA | | |
| | AACTTGGTTGGCAGTTGGAAGAAATGAATTACATGGCGTCCGAGGGCGAGGGCGAGGTGTGTGTGAA | | |
| | AGGGCCAAATGTATTTCAGGGCTACTTGAAGGACCCAGCGAAAACAGCAGAAGCTTTGGACAAAGAC | | |
| | GGCTGGTTACACACAGGGGACATCGGAAAATGGTTACCAAATGGCACCTTGAAAATTATCGACCGGA | | |
| | AAAAGCACATATTTAAGCTGGCACAAGGAGAATACATAGCCCCTGAAAAGATTGAAAATATCTACAT | | |
| | GCGAAGTGAGCCTGTTGCTCAGGTGTTTGTCCACGGAGAAAGCCTGCAGGCATTTCTCATTGCAATT | | |
| | GTGGTACCAGATGTTGAGACATTATGTTCCTGGGCCCAAAAGAGAGGATTTGAAGGGTCGTTTGAGG | | |
| | AACTGTGCAGAAATAAGGATGTCAAAAAAGCTATCCTCGAAGATATGGTGAGACTTGGGAAGGATTC | | |
| | TGGTCTGAAACCATTTGAACAGGTCAAAGGCATCACATTGCACCCTGAATTATTTTCTATCGACAAT | | |
| | GGCCTTCTGACTCCAACAATGAAGGCGAAAAGGCCAGAGCTGCGGAACTATTTCAGGTCGCAGATAG | | |
| | ATGACCTCTATTCCATCATCAAGGTTTAGTGTGAAGAAGAAAGCTCAGAGGAAATGGCACAGTTCCA | | |
| | CAATCTCTTCTCCTGCTGATGGCCTTCATGTTGTTAATTTTGAATACAGCAAGTGTAGGGAAGGAAG | | |
| | CGTTCTGTGTTTGACTTGTCCATTCGGGGTTCTTCTCATAGGAATGCTAGAGGAAACAGAACACTGC | | |
| | CTTACAGTCACCTCAGTGTTCAGACCATGTTTATGGTAATACACACTTCCAAAAGTAGCCTTAAAAA | | |
| | TTGTAAAGGGATACTATAAATGTGCTAATTATTTGAGACTTCCTCAGTTTAAAAAGTGGGTTTTAAA | | |
| | TCTTCTGTCTCCCTGTTTTTCTAATCAAGGGGTTAGGACTTTGCTATCTCTGAGATGTCTGCTACTT | | |
| | CGTCGAAATTCTGCAGCTGTCTGCTGCTCTAAAGAGTACAGTGCTCTAGAGGGAAGTGTTCCCTTTA | | |
| | AAAATAAGAACAACTGTCCTGGCTGGAGATCTCACAAGCGGACCAGAGATCTTTTTAAATCCCTGCT | | |
| | ACTGTCCCTTCTCACAGGCATTCACAGAACCCTTCTGATTCGAAGGGTTACGAAACTCATGTTCTTC | | |
| | TCCAGTCCCCTGTGGTTTCTGTTGGAGCATAAGGTTTCCAGTAAGCGGGAGGGCAGATCCAACTCAG | | |
| | AACCATGCAGATAAGGAGCCTCTGGCAAATGGGTGCTGCATCAGAACGCGTGGATTCTCTTTCATGG | | |
| | CAGATGCTCTTGGACTCGGTTCTCCAGGCCTGATTCCCCGACTCCATCCTTTTTCAGGGTTATTTAA | | |
| | AAATCTGCCTTAGATTCTATAGTGAAGACAAGCATTTCAAGAAAGAGTTACCTGGATCAGCCATGCT | | |
| | CAGCTGTGACGCCTGATAACTGTCTACTTTATCTTCACTGAACCACTCACTCTGTGTAAAGGCCAAC | | |
| | GGATTTTTAATGTGGTTTTCATATCAAAAGATCATGTTGGGATTAACTTGCCTTTTTCCCCAAAAAA | | |
| | TAAACTCTCAGGCAAGGCATTTCTTTTAAAGCTATTCCG | | |
| | ORF Start: ATG at 74 | | ORF Stop: TAG at 2171 |
| | SEQ ID NO:112 | 699 aa | MW at 78347.0 kD |

| | |
|---|---|
| NOV8b, | MQAHELFRYFRMPELVDFRQCVTLPTNTLMGFGAFSRRLTTFWRPRHPKPLKPPWHLSMQSVEVAGS |
| CG148278-01 | |
| Protein | GGARRSALLDSDEPLVYFYDDVTTLYEGFQRGIQVSNNGPCLGSRKPDQPYEWLSYKQVAELSECIG |
| Sequence | |
| | SALIQKGFKTAPDQFIGIFAQNRPEWVIIEQGCFAYSMVIVPLYDTLGNEAITYIVNKAELSLVFVD |
| | KPEKAKLLLEGVENKLIPGLKIIVVMDSYGSELVERGQRCGVEVTSMKAMEDLGRANRRKPKPPAPE |
| | DLAVICFTSGTTGNPKGAMVTHRNIVSDCSAFVKATENTVNPCPDDTLISFLPLAHMFERVVECVML |
| | CHGAKIGFFQGDIRLLMDDLKVLQPTVFPVVPRLLNRMFDRIFGQANTTVKRWLLDFASKRKEADVR |
| | SGIIRNNSLWDRLIFHKVQSSLGGRVRLMVTGAAPVSATVLTFLRAALGCQFYEGYGQTECTAGCCL |
| | TMPGDWTTGHVGAPMPCNLIKLGWQLEEMNYMASEGEGEVCVKGPNVFQGYLKDPAKTAEALDKDGW |
| | LHTGDIGKWLPNGTLKIIDRKKHIFKLAQGEYIAPEKIENIYMRSEPVAQVFVHGESLQAFLIAIVV |
| | PDVETLCSWAQKRGFEGSFEELCRNKDVKKAILEDMVRLGKDSGLKPFEQVKGITLHPELFSIDNGL |
| | LTPTMKAKRPELRNYFRSQIDDLYSIIKV |

Sequence comparison of the above protein sequences yields the following sequence relationships shown in Table 8B.

TABLE 8B
Comparison of NOV8a against NOV8b.

| | NOV8a | Identities/ |
|---|---|---|
| | Residues/ | Similarities for |
| Protein | Match | the Matched |
| Sequence | Residues | Region |
| NOV8b | 5 . . . 702 | 679/700 (97%) |
| | 1 . . . 699 | 686/700 (98%) |

Further analysis of the NOV8a protein yielded the following properties shown in Table 8C.

TABLE 8C
Protein Sequence Properties NOV8a

| SignalP | | | | |
|---|---|---|---|---|
| analysis: | Cleavage site between residues 53 and 54 | | | |
| PSORT II | PSG: a new signal peptide prediction method | | | |
| analysis: | N-region: length 9; pos.chg 1; neg.chg 1 | | | |
| | H-region: length 2; peak value −6.22 | | | |
| | PSG score: −10.62 | | | |
| | GvH: von Heijne's method for signal seq. recognition | | | |
| | GvH score (threshold: −2.1): −3.91 | | | |
| | possible cleavage site: between 52 and 53 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 3 | | | |
| | Number of TMS(s) for threshold 0.5: 1 | | | |
| | INTEGRAL Likelihood = −2.28 Transmembrane | | | |
| | 590-606 | | | |
| | PERIPHERAL Likelihood = 2.92 (at 323) | | | |
| | ALOM score: −2.28 (number of TMSs: 1) | | | |
| | MTOP: Prediction of membrane topology (Hartmann et al.) | | | |
| | Center position for calculation: 597 | | | |
| | Charge difference: −0.5 C(−1.0) − N(−0.5) | | | |
| | N >= C: N-terminal side will be inside | | | |
| | >>> Single TMS is located near the C-terminus | | | |
| | >>> membrane topology: type Nt (cytoplasmic tail 1 to 589) | | | |
| | MITDISC: discrimination of mitochondrial targeting seq | | | |
| | R content: | 3 | Hyd Moment (75): | 6.84 |
| | Hyd Moment(95): | 5.71 | G content: | 1 |
| | D/E content: | 2 | S/T content: | 2 |

```
Score: −4.13
Gavel: prediction of cleavage sites for mitochondrial
preseq cleavage site motif not found
NUCDISC: discrimination of nuclear localization signals
pat4: RRKP (4) at 263
pat4: RKPK (4) at 264
pat4: RKKH (3) at 559
pat7: PTMKAKR (3) at 676
bipartite: none
content of basic residues: 11.7%
NLS Score: 0.47
KDEL: ER retention motif in the C-terminus: none
ER Membrane Retention Signals:
KKXX-like motif in the C-terminus: SIIK
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: too long tail
Dileucine motif in the tail: found
LL at 79
LL at 212
LL at 213
LL at 354
LL at 373
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
--------------------------
Final Results (k = 9/23):
30.4%: cytoplasmic
26.1%: nuclear
13.0%: Golgi
13.0%: mitochondrial
  8.7%: endoplasmic reticulum
  4.3%: vesicles of secretory system
  4.3%: peroxisomal
>> prediction for CG148278-02 is cyt (k = 23)
```

A search of the NOV8a protein against the Geneseq database, a proprietary database that contains sequences published in patents and patent publication, yielded several homologous proteins shown in Table 8D.

TABLE 8D
Geneseq Results for NOV8a

| | | | Identities/ | |
|---|---|---|---|---|
| | | | Similarities for | |
| Geneseq | Protein/Organism/Length | NOV8a Residues/ | the Matched | Expect |
| Identifier | [Patent #, Date] | Match Residues | Region | Value |
| ABP65246 | Hypoxia-regulated protein | 5 . . . 702 | 697/698 (99%) | 0.0 |
| | #120 - Homo sapiens, 698 aa. | 1 . . . 698 | 697/698 (99%) | |
| | [WO200246465-A2, | | | |
| | 13 JUN. 2002] | | | |
| AAU74393 | Human cDNA encoding | 5 . . . 702 | 697/698 (99%) | 0.0 |
| | ovarian tumour protein clone | 1 . . . 698 | 697/698 (99%) | |
| | OVM-65 - Homo sapiens, | | | |
| | 698 aa. [WO200190154-A2, | | | |
| | 29 NOV. 2001] | | | |
| AAB42827 | Human ORFX ORF2591 | 5 . . . 699 | 464/695 (66%) | 0.0 |
| | polypeptide sequence SEQ ID | 1 . . . 695 | 569/695 (81%) | |
| | NO: 5182 - Homo sapiens, | | | |
| | 697 aa. [WO200058473-A2, | | | |
| | 05 OCT. 2000] | | | |
| ABG65301 | Human albumin fusion | 29 . . . 701 | 414/673 (61%) | 0.0 |
| | protein #1976 - Homo | 12 . . . 682 | 527/673 (77%) | |
| | sapiens, 683 aa. | | | |
| | [WO200177137-A1, | | | |
| | 18 OCT. 2001] | | | |
| AAU77791 | Human PRO1250 protein - | 29 . . . 701 | 414/673 (61%) | 0.0 |
| | Homo sapiens, 739 aa. | 68 . . . 738 | 527/673 (77%) | |
| | [WO200149715-A2, | | | |
| | 12 JUL. 2001] | | | |

In a BLAST search of public sequence datbases, the NOV8a protein was found to have homology to the proteins shown in the BLASTP data in Table 8E.

TABLE 8E
Public BLASTP Results for NOV8a

| | | | Identities/ | |
|---|---|---|---|---|
| Protein | | | Similarities for | |
| Accession | | NOV8a Residues/ | the Matched | Expect |
| Number | Protein/Organism/Length | Match Residues | Portion | Value |
| P33121 | Long-chain-fatty-acid--CoA | 5 . . . 702 | 697/698 (99%) | 0.0 |
| | ligase 2 (EC 6.2.1.3) | 1 . . . 698 | 697/698 (99%) | |
| | (Long-chain acyl-CoA | | | |
| | synthetase 2) (LACS 2) - | | | |
| | Homo sapiens (Human), 698 | | | |
| | aa. | | | |
| P41215 | Long-chain-fatty-acid--CoA | 5 . . . 702 | 679/700 (97%) | 0.0 |
| | ligase 1 (EC 6.2.1.3) | 1 . . . 699 | 686/700 (98%) | |
| | (Long-chain acyl-CoA | | | |
| | synthetase 1) (LACS 1) | | | |
| | (Palmitoyl-CoA ligase) - | | | |
| | Homo sapiens (Human), 699 | | | |
| | aa. | | | |
| Q9GLP3 | Long-chain fatty acid CoA | 5 . . . 702 | 649/698 (92%) | 0.0 |
| | ligase (EC 6.2.1.3) - Callithrix | 1 . . . 698 | 677/698 (96%) | |
| | jacchus (Common marmoset), | | | |
| | 698 aa. | | | |
| Q9JID6 | Long-chain-fatty-acid--CoA | 5 . . . 702 | 618/698 (88%) | 0.0 |
| | ligase 1 (EC 6.2.1.3) | 1 . . . 698 | 662/698 (94%) | |
| | (Long-chain acyl-CoA | | | |
| | synthetase 1) (LACS 1) | | | |
| | (Palmitoyl-CoA ligase) - Cavia | | | |
| | porcellus (Guinea pig), 698 aa. | | | |
| P18163 | Long-chain-fatty-acid--CoA | 5 . . . 702 | 597/699 (85%) | 0.0 |
| | ligase, liver isozyme (EC | 1 . . . 699 | 657/699 (93%) | |
| | 6.2.1.3) (Long-chain acyl-CoA | | | |
| | synthetase 2) (LACS 2) - | | | |
| | Rattus norvegicus (Rat), 699 | | | |
| | aa. | | | |

PFam analysis predicts that the NOV8a protein contains the domains shown in the Table 8F.

TABLE 8F
Domain Analysis of NOV8a

| | | Identities/ | |
|---|---|---|---|
| | | Similarities for | |
| | NOV8a Match | the Matched | Expect |
| Pfam Domain | Region | Region | Value |
| AMP-binding | 126 . . . 592 | 108/478 (23%) | 9.8e−106 |
| | | 346/478 (72%) | |

### Example 9

The NOV9 clone was analyzed, and the nucleotide and encoded polypeptide sequences are shown in Table 9A.

TABLE 9A
NOV9 Sequence Analysis

| | SEQ ID NO:113 | 975 bp | |
|---|---|---|---|
| NOV9a, | CTGCCTGCTTAGGAGGTTGTAGAAAGCTCTGTAGGTTCTCTCTGTGTGTCCTACAGGAGTCTTCAGGC | | |
| CG152981-01 | | | |
| DNA Sequence | CAGCTCCCTGTCGGATGGCTTTTATGAAAAAATATCTCCTCCCCATTCTGGGGCTCTTCATGGCCTAC | | |
| | TACTACTATTCTGCAAACGAGGAATTCAGACCAGAGATGCTCCAAGGAAAGAAAGTGATTGTCACAGG | | |
| | GGCCAGCAAAGGGATCGGAAGAGAGATGGCTTATCATCTGGCGAAGATGGGAGCCCATGTGGTGGTGA | | |
| | CAGCGAGGTCAAAAGAAACTCTACAGAAGGTGGTATCCCACTGCCTGGAGCTTGGAGCAGCCTCAGCA | | |
| | CACTACATTGCTGGCACCATGGAAGACATGACCTTCGCAGAGCAATTTGTTGCCCAAGCAGGAAAGCT | | |
| | CATGGGAGGACTAGACATGCTCATTCTCAACCACATCACCAACACTTCTTTGAATCTTTTTCATGATG | | |
| | ATATTCACCATGTGCGCAAAAGCATGGAAGTCAACTTCCTCAGTTACGTGGTCCTGACTGTAGCTGCC | | |
| | TTGCCCATGCTGAAGCAGAGCAATGGAAGCATTGTTGTCGTCTCCTCTCTGGCTGGGAAAGTGGCTTA | | |
| | TCCAATGGTTGCTGCCTATTCTGCAAGCAAGTTTGCTTTGGATGGGTTCTTCTCCTCCATCAGAAAGG | | |
| | AATATTCAGTGTCCAGGGTCAATGTATCAATCACTCTCTGTGTTCTTGGCCTCATAGACACAGAAACA | | |
| | GCCATGAAGGCAGTTTCTGGGATAGTCCATATGCAAGCAGCTCCAAAGGAGGAATGTGCCCTGGAGGT | | |
| | CATCAAAGGGGGAGCTCTGCGCCAAGAAGAAGTGTATTATGACAGCTCACTCTGGACCACTCTTCTGA | | |
| | TCAGAAATCCATGCAGGAAGATCCTGGAATTTCTCTACTCAACGAGCTATAATATGGACAGATTCATA | | |
| | AACAAGTAGGAACTCCCTGAGGG | | |
| | ORF Start: ATG at 83 | | ORF Stop: TAG at 959 |
| | SEQ ID NO:114 | 292 aa | MW at 32386.6 kD |
| NOV9a, | MAFMKKYLLPILGLFMAYYYYSANEEFRPEMLQGKKVIVTGASKGIGREMAYHLAKMGAHVVVTARSK | | |
| CG152981-01 | | | |
| Protein | ETLQKVVSHCLELGAASAHYIAGTMEDMTFAEQFVAQAGKLMGGLDMLILNHITNTSLNLFHDDIHHV | | |
| Sequence | | | |
| | RKSMEVNFLSYVVLTVAALPMLKQSNGSIVVVSSLAGKVAYPMVAAYSASKFALDGFFSSIRKEYSVS | | |
| | RVNVSITLCVLGLIDTETAMKAVSGIVHMQAAPKEECALEVIKGGALRQEEVYYDSSLWTTLLIRNPC | | |
| | RKILEFLYSTSYNMDRFINK | | |
| | SEQ ID NO:115 | 831 bp | |
| NOV9b, | CTGCCTGCTTAGGAGGTTGTAGAAAGCTCTGTAGGTTCTCTCTGTGTGTCCTACAGGAGTCTTCAGG | | |
| CG152981-02 | | | |
| DNA Sequence | CCAGCTCCCTGTCGGATGGCTTTTATGAAAAATATCTCCTCCCCATTCTGGGGCTCTTCATGGCCT | | |
| | ACTACTACTATTCTGCAAACGAGGAATTCAGACCAGAGATGCTCCAAGGAAAGAAAGTGATTGTCAC | | |
| | AGGGGCCAGCAAAGGGATCGGAAGAGAGATGGCTTATCATCTGGCGAAGATGGGAGCCCATGTGGTG | | |
| | GTGACAGCGAGGTCAAAAGAAACTCTACAGAAGGTGGTATCCCACTGCCTGGAGCTTGGAGCAGCCT | | |
| | CAGCACACTACATTGCTGGCACCATGGAAGACATGACCTTCGCAGAGCAATTTGTTGCCCAAGCAGG | | |
| | AAAGCTCATGGGAGGACTAGACATGCTCATTCTCAACCACATCACCAACACTTCTTTGAATCTTTTT | | |
| | CATGATGATATTCACCATGTGCGCAAAAGCATGGAAGTCAACTTCCTCAGTTACGTGGTCCTGACTG | | |
| | TAGCTGCCTTGCCCATGCTGAAGCAGAGCAATGGAAGCATTGTTGTCGTCTCCTCTCTGGCTGAAAC | | |
| | AGCCATGAAGGCAGTTTCTGGGATAGTCCATATGCAAGCAGCTCCAAAGGAGGAATGTGCCCTGGAG | | |
| | ATCATCAAAGGGGGAGCTCTGCGCCAAGAAGAAGTGTATTATGACAGCTCACTCTGGACCACTCTTC | | |
| | TGATCAGAAATCCATGCAGGAAGATCCTGGAATTTCTCTACTCAACGAGCTATAATATGGACAGATT | | |
| | CATAAACAAGTAGGAACTCCCTGAGGG | | |
| | ORF Start: ATG at 83 | | ORF Stop: TAG at 815 |
| | SEQ ID NO:116 | 244 aa | MW at 27242.6 kD |

| | |
|---|---|
| NOV9b, | MAFMKKYLLPILGLFMAYYYYSANEEFRPEMLQGKKVIVTGASKGIGREMAYHLAKMGAHVVVTARS |
| CG152981-02 | |
| Protein | KETLQKVVSHCLELGAASAHYIAGTMEDMTFAEQFVAQAGKLMGGLDMLILNHITNTSLNLFHDDIH |
| Sequence | |
| | HVRKSMEVNFLSYVVLTVAALPMLKQSNGSIVVVSSLAETAMKAVSGIVHMQAAPKEECALEIIKGG |
| | ALRQEEVYYDSSLWTTLLIRNPCRKILEFLYSTSYNMDRFINK |

Sequence comparison of the above protein sequences yields the following sequence relationships shown in Table 9B.

TABLE 9B
Comparison of NOV9a against NOV9b.

| | NOV9a | Identities/ |
|---|---|---|
| | Residues/ | Similarities for |
| Protein | Match | the Matched |
| Sequence | Residues | Region |
| NOV9b | 1 . . . 292 | 243/292 (83%) |
| | 1 . . . 244 | 244/292 (83%) |

Further analysis of the NOV9a protein yielded the following properties shown in Table 9C.

TABLE 9C
Protein Sequence Properties NOV9a

| SignalP | | | | |
|---|---|---|---|---|
| analysis: | Cleavage site between residues 24 and 25 | | | |
| PSORT II | PSG: a new signal peptide prediction method | | | |
| analysis: | N-region: length 6; pos.chg 2; neg.chg 0 | | | |
| | H-region: length 18; peak value 10.15 | | | |
| | PSG score: 5.75 | | | |
| | GvH: von Heijne's method for signal seq. recognition | | | |
| | GvH score (threshold: −2.1): −9.92 | | | |
| | possible cleavage site: between 13 and 14 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 3 | | | |
| | Number of TMS(s) for threshold 0.5: 1 | | | |
| | INTEGRAL Likelihood = −2.92 Transmembrane | | | |
| | 142-158 | | | |
| | PERIPHERAL Likelihood = 1.80 (at 1) | | | |
| | ALOM score: −2.92 (number of TMSs: 1) | | | |
| | MTOP: Prediction of membrane topology (Hartmann et al.) | | | |
| | Center position for calculation: 149 | | | |
| | Charge difference: 0.5 C(1.0)-N(0.5) | | | |
| | C > N: C-terminal side will be inside | | | |
| | >>>Caution: Inconsistent mtop result with signal peptide | | | |
| | >>> membrane topology: type 1b (cytoplasmic tail | | | |
| | 142 to 292) | | | |
| | MITDISC: discrimination of mitochondrial targeting seq | | | |
| | R content: | 0 | Hyd Moment(75): | 9.69 |
| | Hyd Moment(95): | 8.03 | G content: | 1 |
| | D/E content: | 1 | S/T content: | 1 |

```
Score: −4.45
Gavel: prediction of cleavage sites for mitochondrial
preseq
cleavage site motif not found
NUCDISC: discrimination of nuclear localization signals
pat4: none
pat7: none
bipartite: none
content of basic residues: 9.9%
NLS Score: −0.47
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: too long tail
Dileucine motif in the tail: found
LL at 266
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
Final Results (k = 9/23):
34.8%: nuclear
21.7%: mitochondrial
21.7%: cytoplasmic
  8.7%: vesicles of secretory system
  4.3%: vacuolar
  4.3%: endoplasmic reticulum
  4.3%: peroxisomal
>> prediction for CG152981-01 is nuc (k = 23)
```

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM79592 | Human protein SEQ ID NO | 1 . . . 292 | 291/292 (99%) | e−164 |
| | 3238 - Homo sapiens, 308 aa. | 17 . . . 308 | 292/292 (99%) | |
| | [W0200157190-A2, | | | |
| | 09 AUG. 2001] | | | |
| AAO14408 | Rat corticosteroid 11-beta | 4 . . . 291 | 219/288 (76%) | e−123 |
| | dehydrogenase enzyme - | 1 . . . 286 | 254/288 (88%) | |
| | Rattus sp, 287 aa. | | | |
| | [W0200202797-A2, | | | |
| | 10 JAN. 2002] | | | |
| AAU99344 | Human short-chain | 4 . . . 258 | 125/256 (48%) | 1e−57 |
| | dehydrogenase/reductase, | 1 . . . 253 | 162/256 (62%) | |
| | 25206, protein - Homo | | | |
| | sapiens, 286 aa. | | | |
| | [WO200244356-A2, | | | |
| | 06 JUN. 2002] | | | |
| ABG16187 | Novel human diagnostic | 140 . . . 252 | 111/113 (98%) | 6e−55 |
| | protein #16178 - Homo | 1 . . . 113 | 112/113 (98%) | |
| | sapiens, 119 aa. | | | |
| | [WO200175067-A2, | | | |
| | 11 OCT. 2001] | | | |
| ABG16188 | Novel human diagnostic | 11 . . . 174 | 107/164 (65%) | 1e−45 |
| | protein #16179 - Homo | 11 . . . 174 | 115/164 (69%) | |
| | sapiens, 203 aa. | | | |
| | [WO200175067-A2, | | | |
| | 11 OCT. 2001] | | | |

In a BLAST search of public sequence datbases, the NOV9a protein was found to have homology to the proteins shown in the BLASTP data in Table 9E.

TABLE 9E
Public BLASTP Results for NOV9a

| | | NOV9a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P28845 | Corticosteroid | 1 . . . 292 | 291/292 (99%) | e−164 |
| | 11-beta-dehydrogenase, | 1 . . . 292 | 292/292 (99%) | |
| | isozyme 1 (EC 1.1.1.146) | | | |
| | (11-DH) | | | |
| | (11-beta-hydroxysteroid | | | |
| | dehydrogenase 1) | | | |
| | (11-beta-HSD1) - Homo | | | |
| | sapiens (Human), 292 aa. | | | |
| Q29608 | Corticosteroid | 1 . . . 291 | 264/291 (90%) | e−148 |
| | 11-beta-dehydrogenase, | 1 . . . 291 | 276/291 (94%) | |
| | isozyme 1 (EC 1.1.1.146) | | | |
| | (11-DH) | | | |
| | (11-beta-hydroxysteroid | | | |
| | dehydrogenase 1) | | | |
| | (11-beta-HSD1) - Saimiri | | | |
| | sciureus (Common squirrel | | | |
| | monkey), 291 aa. | | | |
| A55573 | 11beta-hydroxysteroid | 2 . . . 291 | 233/290 (80%) | e−134 |
| | dehydrogenase | 1 . . . 290 | 270/290 (92%) | |
| | (EC 1.1.1.146) - | | | |
| | rabbit, 291 aa. | | | |
| Q95L61 | 11-beta hydroxysteroid | 1 . . . 291 | 238/291 (81%) | e−133 |
| | dehydrogenase isoform 1 - Sus | 1 . . . 291 | 268/291 (91%) | |
| | scrofa (Pig), 292 aa. | | | |
| P51975 | Corticosteroid | 1 . . . 291 | 227/291 (78%) | e−130 |
| | 11-beta-dehydrogenase, | 1 . . . 291 | 269/291 (92%) | |
| | isozyme 1 (EC 1.1.1.146) | | | |
| | (11-DH) | | | |
| | (11-beta-hydroxysteroid | | | |
| | dehydrogenase 1) | | | |
| | (11-beta-HSD1) - Ovis aries | | | |
| | (Sheep), 292 aa. | | | |

PFam analysis predicts that the NOV9a protein contains the domains shown in the Table 9F.

TABLE 9F
Domain Analysis of NOV9a

| | | Identities/ | |
|---|---|---|---|
| | | Similarities | |
| | NOV9a | for the | |
| Pfam | Match | Matched | Expect |
| Domain | Region | Region | Value |
| adh_short | 33 . . . 286 | 74/278 (27%) | 1.8e−65 |
| | | 187/278 (67%) | |

### Example 10

The NOV10 clone was analyzed, and the nucleotide and encoded polypeptide sequences are shown in Table 10A.

TABLE 10A
NOV10 Sequence Analysis

| | SEQ ID NO:117 | 1662 bp | |
|---|---|---|---|
| NOV10a, | TGCAGATCAGTGTGTGAGGGAACTGCCATCATGAGGTCTGACAAGTCAGCTTTGGTATTTCTGCTCC | | |
| CG159035-01 | | | |
| DNA Sequence | TGCAGCTCTTCTGTGTTGGCTGTGGATTCTGTGGGAAAGTCCTGGTGTGGCCCTGTGACATGAGCCA | | |
| | TTGGCTTAATGTCAAGGTCATTCTAGAAGAGCTCATAGTGAGAGGCCATGAGGTAACAGTATTGACT | | |
| | CACTCAAAGCCTTCGTTAATTGACTACAGGAAGCCTTCTGCATTGAAATTTGAGGTGGTCCATATGC | | |
| | CACAGGACAGAACAGAAGAAAATGAAATATTTGTTGACCTAGCTCTGAATGTCTTGCCAGGCTTATC | | |
| | AACCTGGCAATCAGTTATAAAATTAAATGATTTTTTTGTTGAAATAAGAGGAACTTTAAAAATGATG | | |
| | TGTGAGAGCTTTATCTACAATCAGACGCTTATGAAGAAGCTACAGGAAACCAACTACGATGTAATGC | | |
| | TTATAGACCCTGTGATTCCCTGTGGAGACCTGATGGCTGAGTTGCTTGCAGTCCCTTTTGTGCTCAC | | |
| | ACTTAGAATTTCTGTAGGAGGCAATATGGAGCGAAGCTGTGGGAAACTTCCAGCTCCACTTTCCTAT | | |
| | GTACCTGTGCCTATGACAGGACTAACAGACAGAATGACCTTTCTGGAAAGAGTAAAAAATTCAATGC | | |
| | TTTCAGTTTTGTTCCACTTCTGGATTCAGGATTACGACTATCATTTTTGGGAAGAGTTTTATAGTAA | | |
| | GGCATTAGGAAGACCCACTACCTTATTTGAGACAATGGGGAAAGCTGACATATGGCTTATGCGAAAC | | |
| | TCCTGGAATTTTCAGTTTCCTCATCCTTTCTTACCAAACGTTGATTTTGTTGGAGGACTCCACTGCA | | |
| | AACCTGCCAAACCCCTACCTAAGGAAATGGAGGAGTTTGTACAGAGCTCTGGAGAAAATGGTGTTGT | | |
| | GGTGTTTTCTCTGGGGTCAATCATAAGTAACATGACAGCAGAAAGGGCCAATGTAATTGCAACAGCC | | |
| | CTGGCCAAGATCCCACAAAAGGTACTGTGGAGATTTGATGGGAATAAACCAGATGCTTTAGGTCTCA | | |
| | ATACTTGGCTGTACGAGTGGATATCCCAGAATGACCTTCTAGGTCATCCAAAAACCAGAGCTTTTAT | | |
| | AACTCATGGTGGAGCCAATGGCATCTATGAGGCAATCTACCATGGGATCCCTATATTGGGCATTCCA | | |
| | TTGTTTGCCGATCAACCTGATAATATTGCTCACATGAAGGCCAAGGGAGCAGCTGTTAGATTGGACT | | |
| | TCAACACAATGTCGAGTACAGACTTGTTGAATGCACTGAAGACAGTAATTAATGTTCCTTTGTATAA | | |
| | AGAGAGTGTTATGAAATTATCAAGAATTCAACATGATCAACCAGTGAAGCCCCTGGATCGAGCAGTC | | |
| | TTCTGGATTGAATTTGTCATGCGCCACAAAGGAGCCAAACACCTTCGAGTTGCAGCCCGTGACCTCA | | |
| | CCTGGTTCCAGTACCACTCTTTGGATGTGATTGGGTTTCTGCTGGCCTGTGTGGCAACTGTGACATT | | |
| | TATCATCACAAAGTGTTGTCTGTTTTGTTTCTGGAAGTTTACTAGAAAAGTGAAGAAGGAAAAAAGG | | |
| | GATTAGTTATGTCCGACATTTGAAGCTGGAAAACCTGATAGATGGGATGACTTC | | |
| | ORF Start: ATG at 31 | | ORF Stop: TAG at 1612 |
| | SEQ ID NO:118 | 527 aa | MW at 60130.9 kD |

| | |
|---|---|
| NOV10a, | MRSDKSALVFLLLQLFCVGCGFCGKVLVWPCDMSHWLNVKVILEELIVRGHEVTVLTHSKPSLIDYR |
| CG159035-01 | |
| Protein Sequence | KPSALKFEVVHMPQDRTEENEIFVDLALNVLPGLSTWQSVIKLNDFFVEIRGTLKMMCESFIYNQTL |
| | MKKLQETNYDVMLIDPVIPCGDLMAELLAVPFVLTLRISVGGNMERSCGKLPAPLSYVPVPMTGLTD |
| | RMTFLERVKNSMLSVLFHFWIQDYDYHFWEEFYSKALGRPTTLFETMGKADIWLMRNSWNFQFPHPF |
| | LPNVDFVGGLHCKPAKPLPKEMEEFVQSSGENGVVVFSLGSIISNMTAERANVIATALAKIPQKVLW |
| | RFDGNKPDALGLNTWLYEWISQNDLLGHPKTRAFITHGGANGIYEAIYHGIPILGIPLFADQPDNIA |
| | HMKAKGAAVRLDFNTMSSTDLLNALKTVINVPLYKESVMKLSRIQHDQPVKPLDRAVFWIEFVMRHK |
| | GAKHLRVAARDLTWFQYHSLDVIGFLLACVATVTFIITKCCLFCFWKFTRKVKKEKRD |

Further analysis of the NOV10a protein yielded the following properties shown in Table 10B.

TABLE 10B
Protein Sequence Properties NOV10a

| SignalP | | | | |
|---|---|---|---|---|
| analysis: | Cleavage site between residues 24 and 25 | | | |
| PSORT II | PSG: a new signal peptide prediction method | | | |
| analysis: | N-region: length 5; pos.chg 2; neg.chg 1 | | | |
| | H-region: length 19; peak value 10.30 | | | |
| | PSG score: 5.90 | | | |
| | GvH: von Heijne's method for signal seq. recognition | | | |
| | GvH score (threshold: −2.1): 1.27 | | | |
| | possible cleavage site: between 21 and 22 | | | |
| | >>> Seems to have a cleavable signal peptide (1 to 21) | | | |
| | ALOM: Klein et al's method for TM region allocation | | | |
| | Init position for calculation: 22 | | | |
| | Tentative number of TMS(s) for the threshold 0.5: 2 | | | |
| | Number of TMS(s) for threshold 0.5: 1 | | | |
| | INTEGRAL Likelihood = −8.49 Transmembrane | | | |
| | 491-507 | | | |
| | PERIPHERAL Likelihood = 2.92 (at 378) | | | |
| | ALOM score: −8.49 (number of TMSs: 1) | | | |
| | MTOP: Prediction of membrane topology (Hartmann et al.) | | | |
| | Center position for calculation: 10 | | | |
| | Charge difference: −1.5 C( 0.5)-N( 2.0) | | | |
| | N >= C: N-terminal side will be inside | | | |
| | >>> membrane topology: type 1a (cytoplasmic tail | | | |
| | 508 to 527) | | | |
| | MITDISC: discrimination of mitochondrial targeting seq | | | |
| | R content: | 1 | Hyd Moment (75): | 2.63 |
| | Hyd Moment (95): | 5.92 | G content: | 3 |
| | D/E content: | 2 | S/T content: | 2 |

Score: −7.46
Gavel: prediction of cleavage sites for mitochondrial
preseq
R-2 motif at 12 MRS|DK
NUCDISC: discrimination of nuclear localization signals
pat4: none
pat7: none
bipartite: none
content of basic residues: 10.8%
NLS Score: −0.47
KDEL: ER retention motif in the C-terminus: none
ER Membrane Retention Signals:
XXRR-like motif in the N-terminus: RSDK
KKXX-like motif in the C-terminus: KEKR
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
Final Results (k = 9/23):
44.4%: endoplasmic reticulum
22.2%: Golgi
11.1%: plasma membrane
11.1%: vesicles of secretory system
11.1%: extracellular, including cell wall
>> prediction for CG159035-01 is end (k = 9)

A search of the NOV10a protein against the Geneseq database, a proprietary database that contains sequences published in patents and patent publication, yielded several homologous proteins shown in Table 10C.

TABLE 10C
Geneseq Results for NOV10a

| | | | Identities/ | |
|---|---|---|---|---|
| | | | Similarities for | |
| Geneseq | Protein/Organism/Length | NOV10a Residues/ | the Matched | |
| Identifier | [Patent #, Date] | Match Residues | Region | Expect Value |
| AAE15434 | Human drug metabolising | 1 . . . 527 | 447/527 (84%) | 0.0 |
| | enzyme (DME)-1 - Homo | 1 . . . 527 | 482/527 (90%) | |
| | sapiens, 527 aa. | | | |
| | [WO200179468-A2, | | | |
| | 25 OCT. 2001] | | | |
| AAU77927 | Human drug-metabolising | 1 . . . 527 | 447/527 (84%) | 0.0 |
| | enzyme - Homo sapiens, 527 | 1 . . . 527 | 482/527 (90%) | |
| | aa. [WO200218554-A2, | | | |
| | 07 MAR. 2002] | | | |
| AAU29284 | Human PRO polypeptide | 1 . . . 527 | 447/527 (84%) | 0.0 |
| | sequence #261 - Homo | 1 . . . 527 | 482/527 (90%) | |
| | sapiens, 527 aa. | | | |
| | [WO200168848-A2, | | | |
| | 20 SEP. 2001] | | | |
| AAE02188 | Human breast cancer specific | 9 . . . 527 | 367/522 (70%) | 0.0 |
| | gene-2 (BCSG-2) protein - | 8 . . . 529 | 413/522 (78%) | |
| | Homo sapiens, 529 aa. | | | |
| | [WO200137779-A2, | | | |
| | 31 MAY 2001] | | | |
| ABG05523 | Novel human diagnostic | 3 . . . 527 | 360/528 (68%) | 0.0 |
| | protein #5514 - Homo | 6 . . . 533 | 412/528 (77%) | |
| | sapiens, 533 aa. | | | |
| | [WO200175067-A2, | | | |
| | 11 OCT. 2001] | | | |

In a BLAST search of public sequence datbases, the NOV10a protein was found to have homology to the proteins shown in the BLASTP data in Table 10D.

TABLE 10D
Public BLASTP Results for NOV10a

| | | | Identities/ | |
|---|---|---|---|---|
| Protein | | | Similarities for | |
| Accession | | NOV10a Residues/ | the Matched | Expect |
| Number | Protein/Organism/Length | Match Residues | Portion | Value |
| CAD48648 | Sequence 1 from Patent | 1 . . . 527 | 448/527 (85%) | 0.0 |
| | WO0226834 - Homo sapiens | 1 . . . 527 | 483/527 (91%) | |
| | (Human), 527 aa. | | | |
| Q9H6S4 | Hypothetical protein | 79 . . . 527 | 370/449 (82%) | 0.0 |
| | FLJ21934 - Homo sapiens | 1 . . . 449 | 405/449 (89%) | |
| | (Human), 449 aa. | | | |
| Q9R110 | UDP glucuronosyltransferase | 1 . . . 527 | 351/530 (66%) | 0.0 |
| | UGT2A3 - Cavia porcellus | 1 . . . 530 | 428/530 (80%) | |
| | (Guinea pig), 530 aa. | | | |
| O75310 | UDP-glucuronosyltransferase | 9 . . . 527 | 367/522 (70%) | 0.0 |
| | 2B11 precursor, microsomal | 8 . . . 529 | 413/522 (78%) | |
| | (EC 2.4.1.17) (UDPGT)- | | | |
| | Homo sapiens (Human), 529 | | | |
| | aa. | | | |
| JE0200 | orphan | 9 . . . 527 | 366/522 (70%) | 0.0 |
| | UDP-glucuronosyltransferase | 8 . . . 529 | 412/522 (78%) | |
| | (EC 2.4.-.-) - human, 529 aa. | | | |

PFam analysis predicts that the NOV10a protein contains the domains shown in the Table 10E.

TABLE 10E
Domain Analysis of NOV10a

| | | Identities/ | |
|---|---|---|---|
| | | Similarities | |
| | NOV10a | for the | |
| Pfam | Match | Matched | Expect |
| Domain | Region | Region | Value |
| UDPGT | 24 . . . 525 | 303/507 (60%) | 3.9e−290 |
| | | 436/507 (86%) | |

### Example 11

The NOV11 clone was analyzed, and the nucleotide and encoded polypeptide sequences are shown in Table 11A.

TABLE 11A
NOV11 Sequence Analysis

| | SEQ ID NO:119 | 3030 bp | |
|---|---|---|---|
| NOV11a, | GCGGCCGCGGGCGCGGGCGGGCGCGCGGGGGAGCCCGGCCGAGGGATGGGCTGCGCCCCCAGCATCC | | |
| CG159232-01 | | | |
| DNA Sequence | ATGTCTCGCAGAGCGGCGTGATCTACTGCCGGGACTCGGACGAGTCCAGCTCGCCCCGCCAGACCAC | | |
| | CAGCGTGTCGCAGGGCCCGGCGGCACCCCTGCCCGGCCTCTTCGTCCAGACCGACGCCGCCGACGCC | | |
| | ATCCCCCCGAGCCGCGCGTCGGGACCCCCCAGCGTAGCCCGCGTCCGCAGGGCCCGCACCGAGCTGG | | |
| | GCAGCGGTAGCAGCGCGGGTTCCGCAGCCCCCGCCGCGACCACCAGCAGGGGCCGGAGGCGCCACTG | | |
| | CTGCAGCAGCGCCGAGGCCGAGACTCAGACCTGCTACACCAGCGTGAAGCAGGTGTCTTCTGCGGAG | | |
| | GTGCGCATCGGGCCCATGAGACTGACGCAGGACCCTATTCAGGTTTTGCTGATCTTTGCAAAGGAAG | | |
| | ATAGTCAGAGCGATGGCTTCTGGTGGGCCTGCGACAGAGCTGGTTATAGATGCAATATTGCTCGGAC | | |
| | TCCAGAGTCAGCCCTTGAATGCTTTCTTGATAAGCATCATGAAATTATTGTAATTGATCATAGACAA | | |
| | ACTCAGAACTTCGATGCAGAAGCAGTGTGCAGGTCGATCCGGGCCACAAATCCCTCCGAGCACACGG | | |
| | TGATCCTCGCAGTGGTTTCGCGAGTATCGGATGACCATGAAGAGGCGTCAGTCCTTCCTCTTCTCCA | | |
| | CGCAGGCTTCAACAGGAGATTTATGGAGAATAGCAGCATAATTGCTTGCTATAATGAACTGATTCAA | | |
| | ATAGAACATGGGGAAGTTCGCTCCCAGTTCAAATTACGGGCCTGTAATTCAGTGTTTACAGCATTAG | | |
| | ATCACTGTCATGAAGCCATAGAAATAACAAGCGATGACCACGTGATTCAGTATGTCAACCCAGCCTT | | |
| | CGAAAGGATGATGGGCTACCACAAAGGTGAGCTCCTGGGAAAAGAACTCGCTGATCTGCCCAAAAGC | | |
| | GATAAGAACCGGGCAGACCTTCTCGACACCATCAATACATGCATCAAGAAGGGAAAGGAGTGGCAGG | | |
| | GGGTTTACTATGCCAGACGGAAATCCGGGGACAGCATCCAACAGCACGTGAAGATCACCCCAGTGAT | | |
| | TGGCCAAGGAGGGAAAATTAGGCATTTTGTCTCGCTCAAGAAACTGTGTTGTACCACTGACAATAAT | | |
| | AAGCAGATTCACAAGATTCATCGTGATTCAGGAGACAATTCTCAGACAGAGCCTCATTCATTCAGAT | | |
| | ATAAGAACAGGAGGAAAGAGTCCATTGACGTGAAATCGATATCATCTCGAGGCAGTGATGCACCAAG | | |
| | CCTGCAGAATCGTCGCTATCCGTCCATGGCGAGGATCCACTCCATGACCATCGAGGCTCCCATCACA | | |
| | AAGGTTATAAATATAATCAATGCAGCCCAAGAAAACAGCCCAGTCACAGTAGCGGAAGCCTTGGACA | | |
| | GAGTTCTAGAGATTTTACGGACCACAGAACTGTACTCCCCTCAGCTGGGTACCAAAGATGAAGATCC | | |
| | CCACACCAGTGATCTTGTTGGAGGCCTGATGACTGACGGCTTGAGAAGACTGTCAGGAAACGAGTAT | | |
| | GTGTTTACTAAGAATGTGCACCAGAGTCACAGTCACCTTGCAATGCCAATAACCATCAATGATGTTC | | |
| | CCCCTTGTATCTCTCAATTACTTGATAATGAGGAGAGTTGGGACTTCAACATCTTTGAATTGGAAGC | | |
| | CATTACGCATAAAAGGCCATTGGTTTATCTGGGCTTAAAGGTCTTCTCTCGGTTTGGAGTATGTGAG | | |
| | TTTTTAAACTGTTCTGAAACCACTCTTCGGGCCTGGTTCCAAGTGATCGAAGCCAACTACCACTCTT | | |
| | CCAATGCCTACCACAACTCCACCCATGCTGCCGACGTCCTGCACGCCACCGCTTTCTTTCTTGGAAA | | |
| | GGAAAGAGTAAAGGGAAGCCTCGATCAGTTGGATGAGGTGGCAGCCCTCATTGCTGCCACAGTCCAT | | |
| | GACGTGGATCACCCGGGAAGGACCAACTCTTTCCTCTGCAATGCAGGCAGTGAGCTTGCTGTGCTCT | | |
| | ACAATGACACTGCTGTTCTGGAGAGTCACCACACCGCCCTGGCCTTCCAGCTCACGGTCAAGGACAC | | |
| | CAAATGCAACATTTTCAAGAATATTGACAGGAACCATTATCGAACGCTGCGCCAGGCTATTATTGAC | | |
| | ATGGTTTTGGCAACAGAGATGACAAAACACTTTGAACATGTGAATAAGTTTGTGAACAGCATCAACA | | |
| | AGCCAATGGCAGCTGAGATTGAAGGCAGCGACTGTGAATGCAACCCTGCTGGGAAGAACTTCCCTGA | | |
| | AAACCAAATCCTGATCAAACGCATGATGATTAAGTGTGCTGACGTGGCCAACCCATGCCGCCCCTTG | | |
| | GACCTGTGCATTGAATGGGCTGGGAGGATCTCTGAGGAGTATTTTGCACAGACTGATGAAGAGAAGA | | |
| | GACAGGGACTACCTGTGGTGATGCCAGTGTTTGACCGGAATACCTGTAGCATCCCCAAGTCTCAGAT | | |
| | CTCTTTCATTGACTACTTCATAACAGACATGTTTGATGCTTGGGATGCCTTTGCACATCTGCCAGCC | | |
| | CTGATGCAACATTTGGCTGACAACTACAAACACTGGAAGACACTAGATGACCTAAAGTGCAAAAGTT | | |
| | TGAGGCTTCCATCTGACAGCTAAAGCCAAGCCACAGAGGGGGCCTCTTGACCGACAAAGGACACTGT | | |
| | GAATCACAGTAGCGTAAACAAGAGGCCTTCCTTTCTAATGACAATGACAGGTATTGGTGAAGGAGCT | | |
| | AATGTTTAATATTTGACCTTGAATCATTCAAGTCCCCAAATTTCATTCTTAGAAAGTTATGTTCCAT | | |
| | GAAGAAAAATATATGTTCTTTTGAATACTTAATGACAGAACAAATACTTGGCAAACTCCTTTGCTCT | | |
| | GCTGTCATCCTGTGTACCCTTGTCAATCCATGGAGCTGGTTCACTGTAACTAGCAGGCCACAGGAAG | | |
| | CAAAGCCTTGGTGCC | | |
| | ORF Start: ATG at 46 | | ORF Stop: TAA at 2701 |
| | SEQ ID NO:120 | 885 aa | MW at 98977.6 kD |

| | |
|---|---|
| NOV11a, | MGCAPSIHVSQSGVIYCRDSDESSSPRQTTSVSQGPAAPLPGLFVQTDAADAIPPSRASGPPSVARV |
| CG159232-01 | |
| Protein Sequence | RRARTELGSGSSAGSAAPAATTSRGRRRHCCSSAEAETQTCYTSVKQVSSAEVRIGPMRLTQDPIQV |
| | LLIFAKEDSQSDGFWWACDRAGYRCNIARTPESALECFLDKHHEIIVIDHRQTQNFDAEAVCRSIRA |
| | TNPSEHTVILAVVSRVSDDHEEASVLPLLHAGFNRRFMENSSIIACYNELIQIEHGEVRSQFKLRAC |
| | NSVFTALDHCHEAIEITSDDHVIQYVNPAFERMMGYHKGELLGKELADLPKSDKNRADLLDTINTCI |
| | KKGKEWQGVYYARRKSGDSIQQHVKITPVIGQGGKIRHFVSLKKLCCTTDNNKQIHKIHRDSGDNSQ |
| | TEPHSFRYKNRRKESIDVKSISSRGSDAPSLQNRRYPSMARIHSMTIEAPITKVINIINAAQENSPV |
| | TVAEALDRVLEILRTTELYSPQLGTKDEDPHTSDLVGGLMTDGLRRLSGNEYVFTKNVHQSHSHLAM |
| | PITINDVPPCISQLLDNEESWDFNIFELEAITHKRPLVYLGLKVFSRFGVCEFLNCSETTLRAWFQV |
| | IEANYHSSNAYHNSTHAADVLHATAFFLGKERVKGSLDQLDEVAALIAATVHDVDHPGRTNSFLCNA |
| | GSELAVLYNDTAVLESHHTALAFQLTVKDTKCNIFKNIDRNHYRTLRQAIIDMVLATEMTKHFEHVN |
| | KFVNSINKPMAAEIEGSDCECNPAGKNFPENQILIKRMMIKCADVANPCRPLDLCIEWAGRISEEYF |
| | AQTDEEKRQGLPVVMPVFDRNTCSIPKSQISFIDYFITDMFDAWDAFAHLPALMQHLADNYKHWKTL |
| | DDLKCKSLRLPSDS |

Further analysis of the NOV11a protein yielded the following properties shown in Table 11B.

TABLE 11B
Protein Sequence Properties NOV11a

| SignalP | | | | |
|---|---|---|---|---|
| analysis: | No Known Signal Sequence Predicted | | | |
| PSORT II | PSG: a new signal peptide prediction method | | | |
| analysis: | N-region: length 0; pos.chg 0; neg.chg 0 | | | |
| | H-region: length 17; peak value 4.45 | | | |
| | PSG score: 0.05 | | | |
| | GvH: von Heijne's method for signal seq. recognition | | | |
| | GvH score (threshold: −2.1): −6.11 | | | |
| | possible cleavage site: between 52 and 53 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 3.61 (at 573) | | | |
| | ALOM score: 3.61 (number of TMSs: 0) | | | |
| | MTOP: Prediction of membrane topology (Hartmann et al.) | | | |
| | Center position for calculation: 6 | | | |
| | Charge difference: −2.5 C(−1.5)-N(1.0) | | | |
| | N >= C: N-terminal side will be inside | | | |
| | MITDISC: discrimination of mitochondrial targeting seq | | | |
| | R content: | 1 | Hyd Moment (75): | 2.84 |
| | Hyd Moment(95): | 1.65 | G content: | 2 |
| | D/E content: | 1 | S/T content: | 3 |

```
Score: −5.11
Gavel: prediction of cleavage sites for mitochondrial
preseq cleavage site motif not found
NUCDISC: discrimination of nuclear localization signals
pat4: RRRH (3) at 93
pat7: none
bipartite: KKGKEWQGVYYARRKSG at 336
content of basic residues: 11.1%
NLS Score: 0.21
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: MGCAPSI
3rd aa is cysteine (may be palmitylated)
Prenylation motif: none
memYQRL: transport motif from cell surface to Golgi:
none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: nuclear
Reliability: 70.6
COIL: Lupas's algorithm to detect coiled-coil regions
total: 0 residues
Final Results (k = 9/23):
78.3%: nuclear
13.0%: cytoplasmic
  4.3%: mitochondrial
  4.3%: peroxisomal
>> prediction for CG159232-01 is nuc (k = 23)
```

A search of the NOV11a protein against the Geneseq database, a proprietary database that contains sequences published in patents and patent publication, yielded several homologous proteins shown in Table 11C.

TABLE 11C
Geneseq Results for NOV11a

| | | | Identities/ | |
|---|---|---|---|---|
| Geneseq | Protein/Organism/Length | NOV11a Residues/ | Similarities for the | Expect |
| Identifier | [Patent #, Date] | Match Residues | Matched Region | Value |
| ABB09006 | Human phosphodiesterase-2 - | 1 . . . 885 | 885/885 (100%) | 0.0 |
| | Homo sapiens, 885 aa. | 1 . . . 885 | 885/885 (100%) | |
| | [WO200198471-A2, | | | |
| | 27 DEC. 2001] | | | |
| AAM79141 | Human protein SEQ ID NO | 114 . . . 885 | 772/772 (100%) | 0.0 |
| | 1803 - Homo sapiens, 773 | 2 . . . 773 | 772/772 (100%) | |
| | aa. [WO200157190-A2, | | | |
| | 09 AUG. 2001] | | | |
| AAB64411 | Amino acid sequence of | 125 . . . 885 | 761/761 (100%) | 0.0 |
| | human intracellular | 1 . . . 761 | 761/761 (100%) | |
| | signalling molecule | | | |
| | INTRA43 - Homo sapiens, | | | |
| | 761 aa. [WO200077040-A2, | | | |
| | 21 DEC. 2000] | | | |
| AAB11938 | Human cyclic nucleotide | 168 . . . 885 | 718/718 (100%) | 0.0 |
| | phosphodiesterase, | 1 . . . 718 | 718/718 (100%) | |
| | PDE8B(E) - Homo sapiens, | | | |
| | 718 aa. [US6080548-A, | | | |
| | 27 JUN. 2000] | | | |
| AAY27196 | Human cyclic nucleotide | 168 . . . 885 | 718/718 (100%) | 0.0 |
| | phosphodiester PDE8B(E) | 1 . . . 718 | 718/718 (100%) | |
| | amino acid sequence - Homo | | | |
| | sapiens, 718 aa. | | | |
| | [US5932423-A, | | | |
| | 03 AUG. 1999] | | | |

In a BLAST search of public sequence datbases, the NOV11a protein was found to have homology to the proteins shown in the BLASTP data in Table 11D.

TABLE 11D
Public BLASTP Results for NOV11a

| Protein | | | Identities/ | |
|---|---|---|---|---|
| Accession | | NOV11a Residues/ | Similarities for the | Expect |
| Number | Protein/Organism/Length | Match Residues | Matched Portion | Value |
| AAC69564 | CAMP-specific | 1 . . . 885 | 885/885 (100%) | 0.0 |
| | phosphodiesterase 8B1 - | 1 . . . 885 | 885/885 (100%) | |
| | Homo sapiens (Human), 885 | | | |
| | aa. | | | |
| Q8N3T2 | Hypothetical protein - Homo | 43 . . . 885 | 842/843 (99%) | 0.0 |
| | sapiens (Human), 843 aa | 1 . . . 843 | 842/843 (99%) | |
| | (fragment). | | | |
| O95263 | High-affinity cAMP-specific | 227 . . . 885 | 659/659 (100%) | 0.0 |
| | and IBMX-insensitive | 1 . . . 659 | 659/659 (100%) | |
| | 3′,5′-cyclic | | | |
| | phosphodiesterase 8B (EC | | | |
| | 3.1.4.17) - Homo sapiens | | | |
| | (Human), 659 aa (fragment). | | | |
| JE0293 | 3′,5′-cyclic-nucleotide | 227 . . . 885 | 658/659 (99%) | 0.0 |
| | phosphodiesterase (EC | 1 . . . 659 | 658/659 (99%) | |
| | 3.1.4.17) 8B, cAMP- | | | |
| | specific - human, 659 aa | | | |
| | (fragment). | | | |
| Q96T71 | cAMP-specific cyclic | 1 . . . 884 | 551/889 (61%) | 0.0 |
| | nucleotide phosphodiesterase | 1 . . . 829 | 682/889 (75%) | |
| | PDE8A1 - Homo sapiens | | | |
| | (Human), 829 aa. | | | |

PFam analysis predicts that the NOV11a protein contains the domains shown in the Table 11E.

TABLE 11E
Domain Analysis of NOV11a

| | | Identities/ | |
|---|---|---|---|
| | | Similarities | |
| | NOV11a | for the | |
| Pfam | Match | Matched | Expect |
| Domain | Region | Region | Value |
| PAS | 269 . . . 334 | 16/70 (23%) | 3.4e−05 |
| | | 45/70 (64%) | |
| PDEase | 614 . . . 853 | 88/244 (36%) | 1.1e−74 |
| | | 154/244 (63%) | |

### Example 12

The NOV12 clone was analyzed, and the nucleotide and encoded polypeptide sequences are shown in Table 12A.

TABLE 12A
NOV12 Sequence Analysis

| | SEQ ID NO:121 | 989 bp | |
|---|---|---|---|
| NOV12a, | GCGGGCCCGCGAGTCCGAGACCTGTCCCAGGAGCTCCAGCTCACGTGACCTGTCACTGCCTCCCGCC | | |
| CG159251-03 | | | |
| DNA Sequence | GCCTCCTGCCCGCGCCATGACCCAGCCGGTGCCCCGGCTCTCCGTGCCCGCCGCGCTGGCCCTGGGC | | |
| | TCAGCCGCACTGGGCGCCGCCTTCGCCACTGGCCTCTTCCTGGGGAGGCGGTGCCCCCCATGGCGAG | | |
| | GCCGGCGAGAGCAGTGCCTGCTTCCCCCCGAGGACAGCCGCCTGTGGCAGTATCTTCTGAGCCGCTC | | |
| | CATGCGGGAGCACCCGGCGCTGCGAAGCCTGAGGCTGCTGACCCTGGAGCAGCCGCAGGGGGATTCT | | |
| | ATGATGACCTGCGAGCAGGCCCAGCTCTTGGCCAACCTGGCGCGGCTCATCCAGGCCAAGAAGGCGC | | |
| | TGGACCTGGGCACCTTCACGGGCTACTCCGCCCTGGCCCTGGCCCTGGCGCTGCCCGCGGACGGGCG | | |
| | CGTGGTGACCTGCGAGGTGGACGCGCAGCCCCCGGAGCTGGGACGGCCCCTGTGGAGGCAGGCCGAG | | |
| | GCGGAGCACAAGATCGACCTCCGGCTGAAGCCCGCCTTGGAGACCCTGGACGAGCTGCTGGCGGCGG | | |
| | GCGAGGCCGGCACCTTCGACGTGGCCGTGGTGGATGCGGACAAGGAGAACTGCTCCGCCTACTACGA | | |
| | GCGCTGCCTGCAGCTGCTGCGACCCGGAGGCATCCTCGCCGTCCTCAGAGTCCTGTGGCGCGGGAAG | | |
| | GTGCTGCAACCTCCGAAAGGGGACGTGGCGGCCGAGTGTGTGCGAAACCTAAACGAACGCATCCGGC | | |
| | GGGACGTCAGGGTCTACATCAGCCTCCTGCCCCTGGGCGATGGACTCACCTTGGCCTTCAAGATCTA | | |
| | GGGCTGGCCCCTAGTGAGTGGGCTCGAGGGAGGGTTGCCTGGAACCCCAGGAATTGACCCTGAGTT | | |
| | TTAAATTCGAAAATAAAGTGGGGCTGGGACACAAAAAAAAAAAAAAAAAAA | | |
| | ORF Start: ATG at 84 | | ORF Stop: TAG at 870 |
| | SEQ ID NO:122 | 262 aa | MW at 28808.2 kD |
| NOV12a, | MTQPVPRLSVPAALALGSAALGAAFATGLFLGRRCPPWRGRREQCLLPPEDSRLWQYLLSRSMREHP | | |
| CG159251-03 | | | |
| Protein Sequence | ALRSLRLLTLEQPQGDSMMTCEQAQLLANLARLIQAKKALDLGTFTGYSALALALALPADGRVVTCE | | |
| | VDAQPPELGRPLWRQAEAEHKIDLRLKPALETLDELLAAGEAGTFDVAVVDADKENCSAYYERCLQL | | |
| | LRPGGILAVLRVLWRGKVLQPPKGDVAAECVRNLNERIRRDVRVYISLLPLGDGLTLAFKI | | |
| | SEQ ID NO:123 | 787 bp | |
| NOV12b, | CCGCGGGTAGTGCCCCGACAAGGTGGAGCCCGGCGGGCCCGCGAGTCCGAGACCTGTCCCAGGAGCT | | |
| CG159251-01 | | | |
| DNA Sequence | CCAGCTCACGTGACCTGTCACTGCCTCCCGCCGCCTCCTGCCCGCGCCATGACCCAGCCGGTGCCCC | | |
| | GGCTCTCCGTGCCCGCCGCGCTGGCCCTGGGCTCAGCCGCACTGGGCGCCGCCTTCGCCACTGGCCT | | |
| | CTTCCTGGGCACCTTCACGGGCTACTCCGCCCTGGCCCTGGCCCTGGCGCTGCCCGCGGACGGGCGC | | |
| | GTGGTGACCTGCGAGGTGGACGCGCAGCCCCCGGAGCTGGGACGGCCCCTGTGGAGGCAGGCCGAGG | | |
| | CGGAGCACAAGATCGACCTCCGGCTGAAGCCCGCCTTGGAGACCCTGGACGAGCTGCTGGCGGCGGG | | |
| | CGAGGCCGGCACCTTCGACGTGGCCGTGGTGGATGCGGACAAGGAGAACTGCTCCGCCTACTACGAG | | |
| | CGCTGCCTGCAGCTGCTGCGACCCGGAGGCATCCTCGCCGTCCTCAGAGTCCTGTGGCGCGGGAAGG | | |
| | TGCTGCAACCTCCGAAAGGGGACGTGGCGGCCGAGTGTGTGCGAAACCTAAACGAACGCATCCGGCG | | |
| | GGACGTCAGGGTCTACATCAGCCTCCTGCCCCTGGGCGATGGACTCACCTTGGCCTTCAAGATCTAG | | |
| | GGCTGGCCCCTAGTGAGTGGGCTCGAGGGAGGGTTGCCTGGGAACCCCAGGAATTGACCCTGAGTTT | | |
| | TAAATTCGAAAATAAAGTGGGGCTGGGACACAAAAAAAAAAAAAAAAAAA | | |
| | ORF Start: ATG at 116 | | ORF Stop: TAG at 668 |
| | SEQ ID NO:124 | 184 aa | MW at 19714.6 kD |
| NOV12b, | MTQPVPRLSVPAALALGSAALGAAFATGLFLGTFTGYSALALALALPADGRVVTCEVDAQPPELGRP | | |
| CG159251-01 | | | |
| Protein Sequence | LWRQAEAEHKIDLRLKPALETLDELLAAGEAGTFDVAVVDADKENCSAYYERCLQLLRPGGILAVLR | | |
| | VLWRGKVLQPPKGDVAAECVRNLNERIRRDVRVYISLLPLGDGLTLAFKI | | |
| | SEQ ID NO:125 | 989 bp | |
| NOV12c, | GCGGGCCCGCGAGTCCGAGACCTGTCCCAGGAGCTCCAGCTCACGTGACCTGTCACTGCCTCCCGCC | | |
| CG159251-02 | | | |
| DNA Sequence | GCCTCCTGCCCGCGCCATGACCCAGCCGGTGCCCCGGCTCTCCGTGCCCGCCGCGCTGGCCCTGGGC | | |
| | TCAGCCGCACTGGGCGCCGCCTTCGCCACTGGCCTCTTCCTGGGGAGGCGGTGCCCCCCATGGCGAG | | |
| | GCCGGCGAGAGCAGTGCCTGCTTCCCCCCGAGGACAGCCGCCTGTGGCAGTATCTTCTGAGCCGCTC | | |
| | CATGCGGGAGCACCCGGCGCTGCGAAGCCTGAGGCTGCTGACCCTGGAGCAGCCGCAGGGGGATTCT | | |
| | ATGATGACCTGCGAGCAGGCCCAGCTCTTGGCCAACCTGGCGCGGCTCATCCAGGCCAAGAAGGCGC | | |
| | TGGACCTGGGCACCTTCACGGGCTACTCCGCCCTGGCCCTGGCCCTGGCGCTGCCCGCGGACGGGCG | | |
| | CGTGGTGACCTGCGAGGTGGACGCGCAGCCCCCGGAGCTGGGACGGCCCCTGTGGAGGCAGGCCGAG | | |
| | GCGGAGCACAAGATCGACCTCCGGCTGAAGCCCGCCTTGGAGACCCTGGACGAGCTGCTGGCGGCGG | | |
| | GCGAGGCCGGCACCTTCGACGTGGCCGTGGTGGATGCGGACAAGGAGAACTGCTCCGCCTACTACGA | | |
| | GCGCTGCCTGCAGCTGCTGCGACCCGGAGGCATCCTCGCCGTCCTCAGAGTCCTGTGGCGCGGGAAG | | |
| | GTGCTGCAACCTCCGAAAGGGGACGTGGCGGCCGAGTGTGTGCGAAACCTAAACGAACGCATCCGGC | | |
| | GGGACGTCAGGGTCTACATCAGCCTCCTGCCCCTGGGCGATGGACTCACCTTGGCCTTCAAGATCTA | | |
| | GGGCTGGCCCCTAGTGAGTGGGCTCGAGGGAGGGTTGCCTGGGAACCCCAGGAATTGACCCTGAGTT | | |
| | TTAAATTCGAAAATAAAGTGGGGCTGGGACACAAAAAAAAAAAAAAAAAAA | | |
| | ORF Start: ATG at 84 | | ORF Stop: TAG at 870 |
| | SEQ ID NO:126 | 262 aa | MW at 28808.2 kD |

| | |
|---|---|
| NOV12c, | MTQPVPRLSVPAALALGSAALGAAFATGLFLGRRCPPWRGRREQCLLPPEDSRLWQYLLSRSMREHP |
| CG159251-02 | |
| Protein Sequence | ALRSLRLLTLEQPQGDSMMTCEQAQLLANLARLIQAKKALDLGTFTGYSALALALALPADGRVVTCE |
| | VDAQPPELGRPLWRQAEAEHKIDLRLKPALETLDELLAAGEAGTFDVAVVDADKENCSAYYERCLQL |
| | LRPGGILAVLRVLWRGKVLQPPKGDVAAECVRNLNERIRRDVRVYISLLPLGDGLTLAFKI |

Sequence comparison of the above protein sequences yields the following sequence relationships shown in Table 12B.

TABLE 12B
Comparison of NOV12a against NOV12b and NOV12c.

| | | Identities/ |
|---|---|---|
| | NOV12a | Similarities |
| | Residues/ | for the |
| Protein | Match | Matched |
| Sequence | Residues | Region |
| NOV12b | 107 . . . 262 | 155/156 (99%) |
| | 29 . . . 184 | 155/156 (99%) |
| NOV12c | 1 . . . 262 | 262/262 (100%) |
| | 1 . . . 262 | 262/262 (100%) |

Further analysis of the NOV12a protein yielded the following properties shown in Table 12C.

TABLE 12C
Protein Sequence Properties NOV12a

| SignalP | Cleavage site between residues 27 and 28 | | | |
|---|---|---|---|---|
| analysis: | | | | |
| PSORT II | PSG: a new signal peptide prediction method | | | |
| analysis: | N- region: length 7; pos. chg 1; neg. chg 0 | | | |
| | H-region: length 25; peak value 9.00 | | | |
| | PSG score: 4.60 | | | |
| | GvH: von Heijne's method for signal seq. recognition | | | |
| | GvH score (threshold: −2.1): −0.30 | | | |
| | possible cleavage site: between 26 and 27 | | | |
| | >>> Seems to have a cleavable signal peptide (1 to 26) | | | |
| | ALOM: Klein et al's method for TM region allocation | | | |
| | Init position for calculation: 27 | | | |
| | Tentative number of TMS(s) for the threshold 0.5: 0 | | | |
| | number of TMS(s) . . . fixed | | | |
| | PERIPHERAL Likelihood = 0.95 (at 198) | | | |
| | ALOM score: 0.95 (number of TMSs: 0) | | | |
| | MTOP: Prediction of membrane topology (Hartmann et al.) | | | |
| | Center position for calculation: 13 | | | |
| | Charge difference: 2.0 C( 4.0) − N( 2.0) | | | |
| | C > N: C-terminal side will be inside | | | |
| | >>>Caution: Inconsistent mtop result with signal peptide | | | |
| | MITDISC: discrimination of mitochondrial targeting seq | | | |
| | R content: | 6 | Hyd Moment (75): | 8.25 |
| | Hyd Moment (95): | 12.06 | G content: | 5 |
| | D/E content: | 1 | S/T content: | 4 |
| | Score: | 0.30 | | |

```
Gavel: prediction of cleavage sites for mitochondrial
preseq
R-2 motif at 51 GRR|EQ
NUCDISC: discrimination of nuclear localization signals
pat4: none
pat7: PPWRGRR (3) at 36
pat7: PWRGRRE (4) at 37
bipartite: none
content of basic residues: 12.6%
NLS Score: 0.13
KDEL: ER retention motif in the C-terminus: none
ER Membrane Retention Signals:
KKXX-like motif in the C-terminus: LAFK
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 89
COIL: Lupas's algorithm to detect coiled-coil regions
total: 0 residues
Final Results (k = 9/23):
60.9%: mitochondrial
13.0%: endoplasmic reticulum
  8.7%: extracellular, including cell wall
  8.7%: cytoplasmic
  4.3%: vacuolar
  4.3%: Golgi
>> prediction for CG159251-03 is mit (k = 23)
```

A search of the NOV12a protein against the Geneseq database, a proprietary database that contains sequences published in patents and patent publication, yielded several homologous proteins shown in Table 12D.

TABLE 12D
Geneseq Results for NOV12a

| | | | Identities/ | |
|---|---|---|---|---|
| Geneseq | Protein/Organism/Length | NOV12a Residues/ | Similarities for the | Expect |
| Identifier | [Patent #, Date] | Match Residues | Matched Region | Value |
| AAM47928 | Human O-methyltransferase | 1 . . . 262 | 262/262 (100%) | e−149 |
| | family member 25692 - | 1 . . . 262 | 262/262 (100%) | |
| | Homo sapiens, 262 aa. | | | |
| | [WO200183719-A2, | | | |
| | 08 NOV. 2001] | | | |
| AAU86138 | Human PRO1558 | 1 . . . 262 | 262/262 (100%) | e−149 |
| | polypeptide - Homo sapiens, | 1 . . . 262 | 262/262 (100%) | |
| | 262 aa. [WO200153486-A1, | | | |
| | 26 JUL. 2001] | | | |
| AAB66174 | Protein of the invention #86 - | 1 . . . 262 | 262/262 (100%) | e−149 |
| | Unidentified, 262 aa. | 1 . . . 262 | 262/262 (100%) | |
| | [WO200078961-A1, | | | |
| | 28 DEC. 2000] | | | |
| AAY87281 | Human signal peptide | 1 . . . 262 | 262/262 (100%) | e−149 |
| | containing protein HSPP-58 | 1 . . . 262 | 262/262 (100%) | |
| | SEQ ID NO: 58 - Homo | | | |
| | sapiens, 262 aa. | | | |
| | [WO200000610-A2, | | | |
| | 06 JAN. 2000] | | | |
| AAY99425 | Human PRO 1558 (UNQ766) | 1 . . . 262 | 262/262 (100%) | e−149 |
| | amino acid sequence SEQ ID | 1 . . . 262 | 262/262 (100%) | |
| | NO: 306 - Homo sapiens, 262 | | | |
| | aa. [WO200012708-A2, | | | |
| | 09 MAR. 2000] | | | |

In a BLAST search of public sequence datbases, the NOV12a protein was found to have homology to the proteins shown in the BLASTP data in Table 12E.

TABLE 12E
Public BLASTP Results for NOV12a

| Protein | | | Identities/ | |
|---|---|---|---|---|
| Accession | | NOV12a Residues/ | Similarities for the | Expect |
| Number | Protein/Organism/Length | Match Residues | Matched Portion | Value |
| CAD20539 | Sequence 1 from Patent | 1 . . . 262 | 262/262 (100%) | e−149 |
| | WO0183719 - Homo sapiens | 1 . . . 262 | 262/262 (100%) | |
| | (Human), 262 aa. | | | |
| Q8TE79 | Hypothetical protein | 1 . . . 262 | 261/262 (99%) | e−148 |
| | FLJ23841 - Homo sapiens | 1 . . . 262 | 261/262 (99%) | |
| | (Human), 262 aa. | | | |
| Q9D8V1 | 1810030M08Rik protein - | 1 . . . 262 | 224/262 (85%) | e−127 |
| | Mus musculus (Mouse), 262 | 1 . . . 262 | 242/262 (91%) | |
| | aa. | | | |
| Q8YLW7 | O-methyltransferase - | 54 . . . 261 | 104/208 (50%) | 3e−52 |
| | Anabaena sp. (strain PCC | 12 . . . 219 | 141/208 (67%) | |
| | 7120), 220 aa. | | | |
| O85769 | Hypothetical 24.8 kDa | 47 . . . 258 | 99/212 (46%) | 1e−46 |
| | protein - Legionella | 6 . . . 214 | 135/212 (62%) | |
| | pneumophila, 218 aa. | | | |

PFam analysis predicts that the NOV12a protein contains the domains shown in the Table 12F.

TABLE 12F
Domain Analysis of NOV12a

| | | Identities/ | |
|---|---|---|---|
| | | Similarities | |
| | NOV12a | for the | |
| | Match | Matched | Expect |
| Pfam Domain | Region | Region | Value |
| Methyltransf_3 | 59 . . . 262 | 86/213 (40%) | 3.8e−72 |
| | | 148/213 (69%) | |

### Example 13

The NOV13 clone was analyzed, and the nucleotide and encoded polypeptide sequences are shown in Table 13A.

TABLE 13A
NOV13 Sequence Analysis

| | SEQ ID NO:127 | 1834 bp | |
|---|---|---|---|
| NOV13a, | GTAGTGTGATCACTTCTTACTGCCGCCTCAAGCTTCCAGCCTCAACTCAAGCAATCCTCCCACCTCA | | |
| CG160563-01 | | | |
| DNA Sequence | GCCACCCAAGTGACTGGGACTACAGAGTCTCCTTCTGTCACCCAGGCTAGAGTGAAGTGGCATGATC | | |
| | TCAGCTCACTGCAACCTCCACCTCCTGGATTCAAGCAATCCTCGTCCCTCAGCCTCCCAAGCAGCTG | | |
| | GGACTACAGATTAAGAATGACCCAAAATAAATTAAAGCTTTGTTCCAAAGCCAATGTGTATACTGAA | | |
| | GTGCCTGATGGAGGATGGGGCTGGGCGGTAGCTGTTTCATTTTTCTTCGTTGAAGTCTTCACCTACG | | |
| | GCATCATCAAGATATTTGGTGTCTTCTTTAATGACTTAATGGACAGTTTTAATGAATCCAATAGCAG | | |
| | GATCTCATGGATAATCTCAATCTGTGTGTTTGTCTTAACATTTTCAGCTCCCCTCGCCACAGTCCTG | | |
| | AGCAATCGTTTCGGACACCGTCTGGTAGTGATGTTGGGGGGGCTACTTGTCAGCACCGGGATGGTGG | | |
| | CCGCCTCCTTCTCACAAGAGGTTTCTCATATGTACGTCGCCATCGGCATCATCTCTGGTCTGGGATA | | |
| | CTGCTTTAGTTTTCTCCCAACTGTAACCATCCTATCACAATATTTTGGCAAAAGACGTTCCATAGTC | | |
| | ACTGCAGTTGCTTCCACAGGAGAATGTTTCGCTGTGTTTGCTTTCGCACCAGCAATCATGGCTCTGA | | |
| | AGGAGCGCATTGGCTGGAGATACAGCCTCCTCTTCGTGGGCCTACTACAGTTAAACATTGTCATCTT | | |
| | CGGAGCACTGCTCAGACCCATCTTTATCAGAGGACCAGCGTCACCGAAAATAGTCATCCAGGAAAAT | | |
| | CGGAAAGAAGCGCAGTATATGCTTGAAAATGAGAAAACACGAACCTCAATAGACTCCATTGACTCAG | | |
| | GAGTAGAACTAACTACCCCACCTAAAAATGTGCCTACTCACACTAACCTGGAACTGGAGCCGAAGGC | | |
| | CGACATGCAGCAGGTCCTGGTGAAGACCAGCCCCAGGCCAAGCGAAAAGAAAGCCCCGCTATTAGAC | | |
| | TTCTCCATTTTGAAAGAGAAAAGTTTTATTTGTTATGCATTATTTGGTCTCTTTGCAACACTGGGAT | | |
| | TCTTTGCACCTTCCTTGTACATCATTCCTCTGGGCATTAGTCTGGGCATTGACCAGGACCGCGCTGC | | |
| | TTTTTTATTATCTACGATGGCCATTGCAGAAGTTTTCGGAAGGATCGGAGCTGGTTTTGTCCTCAAC | | |
| | AGGGAGCCCATTCGTAAGATTTACATTGAGCTCATCTGCGTCATCTTATTGACTGTGTCTCTGTTTG | | |
| | CCTTTACTTTTGCTACTGAATTCTGGGGTCTAATGTCATGCAGCATATTTTTTGGGTTTATGGTTGG | | |
| | AACAATAGGAGGGACTCACATTCCACTGCTTGCTGAGGATGATGTCGTGGGCATTGAGAAGATGTCT | | |
| | TCTGCAGCTGGGGTCTACATCTTCATTCAGAGCATAGCAGGACTGGCTGGACCGCCCCTTGCAGGTT | | |
| | TGTTGGTGGACCAAAGTAAGATCTACAGCAGGGCCTTCTACTCCTGCGCAGCTGGCATGGCCCTGGC | | |
| | TGCTGTGTGCCTCGCCCTGGTGAGACCGTGTAAGATGGGACTGTGCCAGCATCATCACTCAGGTGAA | | |
| | ACAAAGGTAGTGAGCCATCGTGGGAAGACTTTACAGGACATACCTGAAGACTTTCTGGAAATGGATC | | |
| | TTGCAAAAAATGAGCACAGAGTTCACGTGCAAATGGAGCCGGCTATGACACACTTTCTTACAACAACA | | |
| | GCCACTGTGTTGGCTGGAGAGGGAT | | |
| | ORF Start: ATG at 218 | | ORF Stop: TGA at 1787 |
| | SEQ ID NO:128 | 523 aa | MW at 57414.8 kD |
| NOV13a, | MTQNKLKLCSKANVYTEVPDGGWGWAVAVSFFFVEVFTYGIIKIFGVFFNDLMDSFNESNSRISWII | | |
| CG160563-01 | | | |
| Protein Sequence | SICVFVLTFSAPLATVLSNRFGHRLVVMLGGLLVSTGMVAASFSQEVSHMYVAIGIISGLGYCFSFL | | |
| | PTVTILSQYFGKRRSIVTAVASTGECFAVFAFAPAIMALKERIGWRYSLLFVGLLQLNIVIFGALLR | | |
| | PIFIRGPASPKIVIQENRKEAQYMLENEKTRTSIDSIDSGVELTTPPKNVPTHTNLELEPKADMQQV | | |
| | LVKTSPRPSEKKAPLLDFSILKEKSFICYALFGLFATLGFFAPSLYIIPLGISLGIDQDRAAFLLST | | |
| | MAIAEVFGRIGAGFVLNREPIRKIYIELICVILLTVSLFAFTFATEFWGLMSCSIFFGFMVGTIGGT | | |
| | HIPLLAEDDVVGIEKMSSAAGVYIFIQSIAGLAGPPLAGLLVDQSKIYSRAFYSCAAGMALAAVCLA | | |
| | LVRPCKMGLCQHHHSGETKVVSHRGKTLQDIPEDFLEMDLAKNEHRVHVQMEPV | | |
| | SEQ ID NO:129 | 1834 bp | |
| NOV13b, | GTAGTGTGATCACTTCTTACTGCCGCCTCAAGCTTCCAGCCTCAACTCAAGCAATCCTCCCACCTCA | | |
| CG160563-01 | | | |
| DNA Sequence | GCCACCCAAGTGACTGGGACTACAGAGTCTCCTTCTGTCACCCAGGCTAGAGTGAAGTGGCATGATC | | |
| | TCAGCTCACTGCAACCTCCACCTCCTGGATTCAAGCAATCCTCGTCCCTCAGCCTCCCAAGCAGCTG | | |
| | GGACTACAGATTAAGAATGACCCAAAATAAATTAAAGCTTTGTTCCAAAGCCAATGTGTATACTGAA | | |
| | GTGCCTGATGGAGGATGGGGCTGGGCGGTAGCTGTTTCATTTTTCTTCGTTGAAGTCTTCACCTACG | | |
| | GCATCATCAAGATATTTGGTGTCTTCTTTAATGACTTAATGGACAGTTTTAATGAATCCAATAGCAG | | |
| | GATCTCATGGATAATCTCAATCTGTGTGTTTGTCTTAACATTTTCAGCTCCCCTCGCCACAGTCCTG | | |
| | AGCAATCGTTTCGGACACCGTCTGGTAGTGATGTTGGGGGGGCTACTTGTCAGCACCGGGATGGTGG | | |
| | CCGCCTCCTTCTCACAAGAGGTTTCTCATATGTACGTCGCCATCGGCATCATCTCTGGTCTGGGATA | | |
| | CTGCTTTAGTTTTCTCCCAACTGTAACCATCCTATCACAATATTTTGGCAAAAGACGTTCCATAGTC | | |
| | ACTGCAGTTGCTTCCACAGGAGAATGTTTCGCTGTGTTTGCTTTCGCACCAGCAATCATGGCTCTGA | | |
| | AGGAGCGCATTGGCTGGAGATACAGCCTCCTCTTCGTGGGCCTACTACAGTTAAACATTGTCATCTT | | |
| | CGGAGCACTGCTCAGACCCATCTTTATCAGAGGACCAGCGTCACCGAAAATAGTCATCCAGGAAAAT | | |
| | CGGAAAGAAGCGCAGTATATGCTTGAAAATGAGAAAACACGAACCTCAATAGACTCCATTGACTCAG | | |
| | GAGTAGAACTAACTACCCCACCTAAAAATGTGCCTACTCACACTAACCTGGAACTGGAGCCGAAGGC | | |
| | CGACATGCAGCAGGTCCTGGTGAAGACCAGCCCCAGGCCAAGCGAAAAGAAAGCCCCGCTATTAGAC | | |
| | TTCTCCATTTTGAAAGAGAAAAGTTTTATTTGTTATGCATTATTTGGTCTCTTTGCAACACTGGGAT | | |
| | TCTTTGCACCTTCCTTGTACATCATTCCTCTGGGCATTAGTCTGGGCATTGACCAGGACCGCGCTGC | | |
| | TTTTTTATTATCTACGATGGCCATTGCAGAAGTTTTCGGAAGGATCGGAGCTGGTTTTGTCCTCAAC | | |
| | AGGGAGCCCATTCGTAAGATTTACATTGAGCTCATCTGCGTCATCTTATTGACTGTGTCTCTGTTTG | | |
| | CCTTTACTTTTGCTACTGAATTCTGGGGTCTAATGTCATGCAGCATATTTTTTGGGTTTATGGTTGG | | |
| | AACAATAGGAGGGACTCACATTCCACTGCTTGCTGAGGATGATGTCGTGGGCATTGAGAAGATGTCT | | |
| | TCTGCAGCTGGGGTCTACATCTTCATTCAGAGCATAGCAGGACTGGCTGGACCGCCCCTTGCAGGTT | | |
| | TGTTGGTGGACCAAAGTAAGATCTACAGCAGGGCCTTCTACTCCTGCGCAGCTGGCATGGCCCTGGC | | |
| | TGCTGTGTGCCTCGCCCTGGTGAGACCGTGTAAGATGGGACTGTGCCAGCATCATCACTCAGGTGAA | | |
| | ACAAAGGTAGTGAGCCATCGTGGGAAGACTTTACAGGACATACCTGAAGACTTTCTGGAAATGGATC | | |
| | TTGCAAAAAATGAGCACAGAGTTCACGTGCAAATGGAGCCGGTATGACACACTTTCTTACAACAACA | | |
| | GCCACTGTGTTGGCTGGAGAGGGAT | | |
| | ORF Start: ATG at 218 | | ORF Stop: TGA at 1787 |
| | SEQ ID NO:130 | 523 aa | MW at 57414.8 kD |

| | |
|---|---|
| NOV13b, | MTQNKLKLCSKANVYTEVPDGGWGWAVAVSFFFVEVFTYGIIKIFGVFFNDLMDSFNESNSRISWII |
| CG160563-01 | |
| Protein Sequence | SICVFVLTFSAPLATVLSNRFGHRLVVMLGGLLVSTGMVAASFSQEVSHMYVAIGIISGLGYCFSFL |
| | PTVTILSQYFGKRRSIVTAVASTGECFAVFAFAPAIMALKERIGWRYSLLFVGLLQLNIVIFGALLR |
| | PIFIRGPASPKIVIQENRKEAQYMLENEKTRTSIDSIDSGVELTTPPKNVPTHTNLELEPKADMQQV |
| | LVKTSPRPSEKKAPLLDFSILKEKSFICYALFGLFATLGFFAPSLYIIPLGISLGIDQDRAAFLLST |
| | MAIAEVFGRIGAGFVLNREPIRKIYIELICVILLTVSLFAFTFATEFWGLMSCSIFFGFMVGTIGGT |
| | HIPLLAEDDVVGIEKMSSAAGVYIFIQSIAGLAGPPLAGLLVDQSKIYSRAFYSCAAGMALAAVCLA |
| | LVRPCKMGLCQHHHSGETKVVSHRGKTLQDIPEDFLEMDLAKNEHRVHVQMEPV |

Sequence comparison of the above protein sequences yields the following sequence relationships shown in Table 13B.

TABLE 13B
Comparison of NOV13a against NOV13b.

| | NOV13a | Identities/ |
|---|---|---|
| | Residues/ | Similarities |
| Protein | Match | for the |
| Sequence | Residues | Matched Region |
| NOV13b | 1 . . . 523 | 523/523 (100%) |
| | 1 . . . 523 | 523/523 (100%) |

Further analysis of the NOV13a protein yielded the following properties shown in Table 13C.

TABLE 13C
Protein Sequence Properties NOV13a

| SignalP | Cleavage site between residues 41 and 42 | | | | |
|---|---|---|---|---|---|
| analysis: | | | | | |
| PSORT II | PSG: a new signal peptide prediction method | | | | |
| analysis: | N-region: length 11; pos.chg 3; neg.chg 0 | | | | |
| | H-region: length 5; peak value −3.08 | | | | |
| | PSG score: −7.48 | | | | |
| | GvH: von Heijne's method for signal seq. recognition | | | | |
| | GvH score (threshold: −2.1): −7.41 | | | | |
| | possible cleavage site: between 38 and 39 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 10 | | | | |
| | INTEGRAL | Likelihood = | −3.72 | Transmembrane | 26-42 |
| | INTEGRAL | Likelihood = | −5.15 | Transmembrane | 66-82 |
| | INTEGRAL | Likelihood = | −5.20 | Transmembrane | 92-108 |
| | INTEGRAL | Likelihood = | −1.54 | Transmembrane | 118-134 |
| | INTEGRAL | Likelihood = | −1.17 | Transmembrane | 150-166 |
| | INTEGRAL | Likelihood = | −6.85 | Transmembrane | 183-199 |
| | INTEGRAL | Likelihood = | −4.30 | Transmembrane | 294-310 |
| | INTEGRAL | Likelihood = | −9.55 | Transmembrane | 363-379 |
| | INTEGRAL | Likelihood = | −0.48 | Transmembrane | 384-400 |
| | INTEGRAL | Likelihood = | −4.30 | Transmembrane | 455-471 |
| | PERIPHERAL | Likelihood = | 0.69 (at 329) | | |
| | ALOM score: −9.55 (number of TMSs: 10) | | | | |
| | MTOP: Prediction of membrane topology (Hartmann et al.) | | | | |
| | Center position for calculation: 33 | | | | |
| | Charge difference: 0.0 C(0.0) − N(0.0) | | | | |
| | N >= C: N-terminal side will be inside | | | | |
| | >>> membrane topology: type 3a | | | | |
| | MITDISC: discrimination of mitochondrial targeting seq | | | | |
| | R content: | 0 | Hyd Moment(75): | 3.58 | |
| | Hyd Moment(95): | 5.26 | G content: | 0 | |
| | D/E content: | 1 | S/T content: | 3 | |
| | Score: | −4.54 | | | |

Gavel: prediction of cleavage sites for mitochondrial preseq
cleavage site motif not found
NUCDISC: discrimination of nuclear localization signals
pat4: none
pat7: none
bipartite: none
content of basic residues: 8.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
Bacterial regulatory proteins, lysR family signature
(PS00044): *** found ***
TAVASTGECFAVFAFAPAIMALKERI at 152
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
66.7%: endoplasmic reticulum
22.2%: mitochondrial
11.1%: nuclear
>> prediction for CG160563-01 is end (k = 9)

A search of the NOV13a protein against the Geneseq database, a proprietary database that contains sequences published in patents and patent publication, yielded several homologous proteins shown in Table 13D.

TABLE 13D
Geneseq Results for NOV13a

| | | | Identities/ | |
|---|---|---|---|---|
| | | | Similarities for | |
| Geneseq | Protein/Organism/Length | NOV13a Residues/ | the Matched | Expect |
| Identifier | [Patent #, Date] | Match Residues | Region | Value |
| ABJ05556 | Breast cancer-associated | 1 . . . 523 | 521/523 (99%) | 0.0 |
| | protein 21 - Unidentified, 523 | 1 . . . 523 | 521/523 (99%) | |
| | aa. [WO200259377-A2, | | | |
| | 01 AUG. 2002] | | | |
| AAB47977 | BCY5 - Homo sapiens, 523 | 1 . . . 523 | 521/523 (99%) | 0.0 |
| | aa. [WO200221134-A2, | 1 . . . 523 | 521/523 (99%) | |
| | 14 MAR. 2002] | | | |
| ABP65184 | Hypoxia-regulated protein | 171 . . . 306 | 129/136 (94%) | 7e−68 |
| | #58 - Homo sapiens, 136 aa. | 1 . . . 136 | 132/136 (96%) | |
| | [W0200246465-A2, | | | |
| | 13 JUN. 2002] | | | |
| AAE22913 | Human transporter and ion | 3 . . . 475 | 132/479 (27%) | 1e−55 |
| | channel (TRICH) 12 - Homo | 25 . . . 464 | 225/479 (46%) | |
| | sapiens, 516 aa. | | | |
| | [WO200222684-A2, | | | |
| | 21 MAR. 2002] | | | |
| AAE22711 | Human transporter protein - | 16 . . . 475 | 128/464 (27%) | 2e−55 |
| | Homo sapiens, 486 aa. | 10 . . . 434 | 219/464 (46%) | |
| | [WO200222678-A2, | | | |
| | 21 MAR. 2002] | | | |

In a BLAST search of public sequence datbases, the NOV13a protein was found to have homology to the proteins shown in the BLASTP data in Table 13E.

TABLE 13E
Public BLASTP Results for NOV13a

| | | | Identities/ | |
|---|---|---|---|---|
| Protein | | | Similarities for | |
| Accession | | NOV13a Residues/ | the Matched | Expect |
| Number | Protein/Organism/Length | Match Residues | Portion | Value |
| O15403 | Monocarboxylate transporter | 1 . . . 523 | 505/523 (96%) | 0.0 |
| | 7 (MCT 7) (MCT 6) - Homo | 1 . . . 523 | 508/523 (96%) | |
| | sapiens (Human), 523 aa. | | | |
| Q91W47 | Hypothetical 57.3 kDa | 1 . . . 523 | 442/524 (84%) | 0.0 |
| | protein - Mus musculus | 1 . . . 523 | 476/524 (90%) | |
| | (Mouse), 523 aa. | | | |
| JC5507 | monocarboxylate transporter | 19 . . . 474 | 144/459 (31%) | 7e−61 |
| | 3 - chicken, 542 aa. | 18 . . . 446 | 226/459 (48%) | |
| Q90632 | Monocarboxylate transporter | 19 . . . 474 | 143/459 (31%) | 3e−59 |
| | 3 (MCT 3) (Retinal epithelial | 18 . . . 446 | 223/459 (48%) | |
| | membrane protein) - Gallus | | | |
| | gallus (Chicken), 542 aa. | | | |
| A55568 | monocarboxylate transporter | 19 . . . 468 | 138/453 (30%) | 5e−55 |
| | 1 - human, 500 aa. | 14 . . . 437 | 215/453 (46%) | |

PFam analysis predicts that the NOV13a protein contains the domains shown in the Table 13F.

TABLE 13F
Domain Analysis of NOV13a

| | | Identities/ | |
|---|---|---|---|
| | | Similarities | |
| | NOV13a | for the | |
| Pfam | Match | Matched | Expect |
| Domain | Region | Region | Value |
| sugar_tr | 23 . . . 504 | 75/556 (13%) | 0.017 |
| | | 302/556 (54%) | |

### Example 14

The NOV14 clone was analyzed, and the nucleotide and encoded polypeptide sequences are shown in Table 14A.

TABLE 14A
NOV14 Sequence Analysis

| | SEQ ID NO:131 | 3624 bp | |
|---|---|---|---|
| NOV14a, | GAGCGGAGTAACCACAGGGCCTGGGACTGGGGGGTTCCCAGATCCTTGAAGCTCACTCCGCCTCCTC | | |
| CG161527-01 | | | |
| DNA Sequence | ACTCTCACTGCATTTCCCACCTTCCTGTGGGCCTTGCGGCATCTTCATCACTGAGGCACCTGGTTAC | | |
| | GCTTCACCTCTTGTTTCCTGCCCTCACTGCATTCCCTCACCTCTACCTTTTTATCCTTCCACCCTAG | | |
| | GCTTCTCTCCTCCCTCTTCCCTCACTCCTGACTCTTCCTCTTCCCAGCGGACGGCTGGAGGACCGCT | | |
| | CAGTCTCTCCTCTCTCACTTCCCTTCCTCTCTCTCACCTTCACCACCCAACACCTCCCTCCCTGCCT | | |
| | CTTTCTTTCTGCTCCCTCATTCTCTCCCCACCACTCTCTTCTCGTGGCCCCCTTGCCCGCGCGCCCT | | |
| | CTTCCCTTCCCCTTGCCTCACTCTCTCAGCTTTCTTCCCACAGTTGAGCTCGGGCAGCTCTTTCTGG | | |
| | GGATAGCTATGGGGCTTTGGGGGAAGAAAGGGACAGTGGCTCCCCATGACCAGAGTCCAAGACGAAG | | |
| | ACCTAAAAAAGGGCTTATCAAGAAAAAAATGGTGAAGAGGGAAAAACAGAAGCGCAATATGGAGGAA | | |
| | CTGAAGAAGGAAGTGGTCATGGATGATCACAAATTAACCTTGGAAGAGCTGAGCACCAAGTACTCCG | | |
| | TGGACCTGACAAAGGGCCATAGCCACCAAAGGGCAAAGGAAATCCTGACTCGAGGTGGACCCAATAC | | |
| | TGTTACCCCACCCCCCACCACTCCAGAATGGGTCAAATTCTGTAAGCAACTGTTCGGAGGCTTCTCC | | |
| | CTCCTACTATGGACTGGGGCCATTCTCTGCTTTGTGGCCTACAGCATCCAGATATATTTCAATGAGG | | |
| | AGCCTACCAAAGACAACCTCTACCTGAGCATCGTACTGTCCGTCGTGGTCATCGTCACTGGCTGCTT | | |
| | CTCCTATTATCAGGAGGCCAAGAGCTCCAAGATCATGGAGTCTTTTAAGAACATGGTGCCTCAGCAA | | |
| | GCTCTGGTAATTCGAGGAGGAGAGAAGATGCAAATTAATGTACAAGAGGTGGTGTTGGGAGACCTGG | | |
| | TGGAAATCAAGGGTGGAGACCGAGTCCCTGCTGACCTCCGGCTTATCTCTGCACAAGGATGTAAGGT | | |
| | GGACAACTCATCCTTGACTGGGGAGTCAGAACCCCAGAGCCGCTCCCCTGACTTCACCCATGAGAAC | | |
| | CCTCTGGAGACCCGAAACATCTGCTTCTTTTCCACCAACTGTGTGGAAGGAACCGCCCGGGGTATTG | | |
| | TGATTGCTACGGGAGACTCCACAGTGATGGGCAGAATTGCCTCCCTGACGTCAGGCCTGGCGGTTGG | | |
| | CCAGACACCTATCGCTGCTGAGATCGAACACTTCATCCATCTGATCACTGTGGTGGCCGTCTTCCTT | | |
| | GGTGTCACTTTTTTTGCGCTCTCACTTCTCTTGGGCTATGGTTGGCTGGAGGCTATCATTTTTCTCA | | |
| | TTGGCATCATTGTGGCCAATGTGCCTGAGGGGCTGTTGGCCACAGTCACTGTGTGCCTGACCCTCAC | | |
| | AGCCAAGCGCATGGCGCGGAAGAACTGCCTGGTGAAGAACCTGGAGGCGGTGGAGACGCTGGGCTCC | | |
| | ACGTCCACCATCTGCTCAGACAAGACGGGCACCCTCACCCAGAACCGCATGACCGTCGCCCACATGT | | |
| | CGTTTGATATGACCGTGTATGAGGCCGACACCACTGAAGAACAGACTGGAAAAACATTTACCAAGAG | | |
| | CTCTGATACCTGGTTTATGCTGGCCCGAATCGCTGGCCTCTGCAACCGGGCTGACTTTAAGGCTAAT | | |
| | CAGGAGATCCTGCCCATTGCTAAGAGGGCCACAACAGGTGATGCTTCCGAGTCAGCCCTCCTCAAGT | | |
| | TCATCGAGCAGTCTTACAGCTCTGTGGCGGAGATGAGAGAGAAAAACCCCAAGGTGGCAGAGATTCC | | |
| | CTTTAATTCTACCAACAAGTACCAGATGTCCATCCACCTTCGGGAGGACAGCTCCCAGACCCACGTA | | |
| | CTGATGATGAAGGGTGCTCCGGAGAGGATCTTGGAGTTTTGTTCTACCTTTCTTCTGAATGGGCAGG | | |
| | AGTACTCAATGAACGATGAAATGAAGGAAGCCTTCCAAAATGCCTACTTAGAACTGGGAGGTCTGGG | | |
| | GGAACGTGTGCTAGGCTTCTGCTTCTTGAATCTGCCTAGCAGCTTCTCCAAGGGATTCCCATTTAAT | | |
| | ACAGATGAAATAAATTTCCCCATGGACAACCTTTGTTTTGTGGGCCTCATATCCATGATTGACCCTC | | |
| | CCCGAGCTGCAGTGCCTGATGCTGTGAGCAAGTGTCGCAGTGCAGGAATTAAGGTGATCATGGTAAC | | |
| | AGGAGATCATCCCATTACAGCTAAGGCCATTGCCAAGGGTGTGGGCATCATCTCAGAAGGCACTGAG | | |
| | ACGGCAGAGGAAGTCGCTGCCCGGCTTAAGATCCCTATCAGCAAGGTCGATGCCAGTGCTGCCAAAG | | |
| | CCATTGTGGTGCATGGTGCAGAACTGAAGGACATACAGTCCAAGCAGCTTGATCAGATCCTCCAGAA | | |
| | CCACCCTGAGATCGTGTTTGCTCGGACCTCCCCTCAGCAGAAGCTCATCATTGTCGAGGGATGTCAG | | |
| | AGGCTGGGAGCCGTTGTGGCCGTGACAGGTGACGGGGTGAACGACTCCCCTGCGCTGAAGAAGGCTG | | |
| | ACATTGGCATTGCCATGGGCATCTCTGGCTCTGACGTCTCTAAGCAGGCAGCCGACATGATCCTGCT | | |
| | GGATGACAACTTTGCCTCCATCGTCACGGGGGTGGAGGAGGGCCGCCTGATCTTTGACAACCTGAAG | | |
| | AAATCCATCATGTACACCCTGACCAGCAACATCCCCGAGATCACGCCCTTCCTGATGTTCATCATCC | | |
| | TCGGTATACCCCTGCCTCTGGGAACCATAACCATCCTCTGCATTGATCTCGGCACTGACATGGTCCC | | |
| | TGCCATCTCCTTGGCTTATGAGTCAGCTGAAAGCGACATCATGAAGAGGCTTCCAAGGAACCCAAAG | | |
| | ACGGATAATCTGGTGAACCACCGTCTCATTGGCATGGCCTATGGACAGATTGGGATGATCCAGGCTC | | |
| | TGGCTGGATTCTTTACCTACTTTGTAATCCTGGCTGAGAATGGTTTTAGGCCTGTTGATCTGCTGGG | | |
| | CATCCGCCTCCACTGGGAAGATAAATACTTGAATGACCTGGAGGACAGCTACGGACAGCAGTGGACC | | |
| | TATGAGCAACGAAAAGTTGTGGAGTTCACATGCCAAACGGCCTTTTTTGTCACCATCGTGGTTGTGC | | |
| | AGTGGGCGGATCTCATCATCTCCAAGACTCGCCGCAACTCACTTTTCCAGCAGGGCATGAGAAACAA | | |
| | AGTCTTAATATTTGGGATCCTGGAGGAGACACTCTTGGCTGCATTTCTGTCCTACACTCCAGGCATG | | |
| | GACGTGGCCCTGCGAATGTACCCACTCAAGATAACCTGGTGGCTCTGTGCCATTCCCTACAGTATTC | | |
| | TCATCTTCGTCTATGATGAAATCAGAAAACTCCTCATCCGTCAGCACCCGGATGGCTGGGTGGAAAG | | |
| | GGAGACGTACTACTAAACTCAGCAGATGAAGAGCTTCATGTGACACAGGGGTGTTGTGAGAGCTGGG | | |
| | ATGGGG | | |
| | ORF Start: ATG at 478 | | ORF Stop: TAA at 3565 |
| | SEQ ID NO:132 | 1029 aa | MW at 114165.1 kD |

| | |
|---|---|
| NOV14a, | MGLWGKKGTVAPHDQSPRRRPKKGLIKKKMVKREKQKRNMEELKKEVVMDDHKLTLEELSTKYSVDL |
| CG161527-01 | |
| Protein Sequence | TKGHSHQRAKEILTRGGPNTVTPPPTTPEWVKFCKQLFGGFSLLLWTGAILCFVAYSIQIYFNEEPT |
| | KDNLYLSIVLSVVVIVTGCFSYYQEAKSSKIMESFKNMVPQQALVIRGGEKMQINVQEVVLGDLVEI |
| | KGGDRVPADLRLISAQGCKVDNSSLTGESEPQSRSPDFTHENPLETRNICFFSTNCVEGTARGIVIA |
| | TGDSTVMGRIASLTSGLAVGQTPIAAEIEHFIHLITVVAVFLGVTFFALSLLLGYGWLEAIIFLIGI |
| | IVANVPEGLLATVTVCLTLTAKRMARKNCLVKNLEAVETLGSTSTICSDKTGTLTQNRMTVAHMWFD |
| | MTVYEADTTEEQTGKTFTKSSDTWFMLARIAGLCNRADFKANQEILPIAKRATTGDASESALLKFIE |
| | QSYSSVAEMREKNPKVAEIPFNSTNKYQMSIHLREDSSQTHVLMMKGAPERILEFCSTFLLNGQEYS |
| | MNDEMKEAFQNAYLELGGLGERVLGFCFLNLPSSFSKGFPFNTDEINFPMDNLCFVGLISMIDPPRA |
| | AVPDAVSKCRSAGIKVIMVTGDHPITAKAIAKGVGIISEGTETAEEVAARLKIPISKVDASAAKAIV |
| | VHGAELKDIQSKQLDQILQNHPEIVFARTSPQQKLIIVEGCQRLGAVVAVTGDGVNDSPALKKADIG |
| | IAMGISGSDVSKQAADMILLDDNFASIVTGVEEGRLIFDNLKKSIMYTLTSNIPEITPFLMFIILGI |
| | PLPLGTITILCIDLGTDMVPAISLAYESAESDIMKRLPRNPKTDNLVNHRLIGMAYGQIGMIQALAG |
| | FFTYFVILAENGFRPVDLLGIRLHWEDKYLNDLEDSYGQQWTYEQRKVVEFTCQTAFFVTIVVVQWA |
| | DLIISKTRRNSLFQQGMRNKVLIFGILEETLLAAFLSYTPGMDVALRMYPLKITWWLCAIPYSILIF |
| | VYDEIRKLLIRQHPDGWVERETYY |

Further analysis of the NOV14a protein yielded the following properties shown in Table 14B.

TABLE 14B
Protein Sequence Properties NOV14a

| SignalP | | | | | |
|---|---|---|---|---|---|
| analysis: | No Known Signal Sequence Predicted | | | | |
| PSORT II | PSG: a new signal peptide prediction method | | | | |
| analysis: | N-region: length 7; pos.chg 2; neg.chg 0 | | | | |
| | H-region: length 6; peak value −5.29 | | | | |
| | PSG score: −9.69 | | | | |
| | GvH: von Heijne's method for signal seq. recognition | | | | |
| | GvH score (threshold: −2.1): −14.68 | | | | |
| | possible cleavage site: between 15 and 16 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 10 | | | | |
| | INTEGRAL | Likelihood = | −3.24 | Transmembrane | 105-121 |
| | INTEGRAL | Likelihood = | −8.39 | Transmembrane | 138-154 |
| | INTEGRAL | Likelihood = | −10.08 | Transmembrane | 305-321 |
| | INTEGRAL | Likelihood = | −5.63 | Transmembrane | 329-345 |
| | INTEGRAL | Likelihood = | 0.21 | Transmembrane | 705-721 |
| | INTEGRAL | Likelihood = | −6.58 | Transmembrane | 800-816 |
| | INTEGRAL | Likelihood = | −3.19 | Transmembrane | 863-879 |
| | INTEGRAL | Likelihood = | −3.77 | Transmembrane | 926-942 |
| | INTEGRAL | Likelihood = | −2.97 | Transmembrane | 959-975 |
| | INTEGRAL | Likelihood = | −1.33 | Transmembrane | 991-1007 |
| | PERIPHERAL Likelihood = 1.59 (at 582) | | | | |
| | ALOM score: −10.08 (number of TMSs: 10) | | | | |
| | MTOP: Prediction of membrane topology (Hartmann et al.) | | | | |
| | Center position for calculation: 112 | | | | |
| | Charge difference: −3.0 C(−2.0) - N( 1.0) | | | | |
| | N >= C: N-terminal side will be inside | | | | |
| | >>> membrane topology: type 3 a | | | | |
| | MITDISC: discrimination of mitochondrial targeting seq | | | | |
| | R content: | 4 | Hyd Moment(75): | 7.31 | |
| | Hyd Moment(95): | 7.79 | G content: | 4 | |
| | D/E content: | 2 | S/T content: | 2 | |
| | Score: −4.33 | | | | |
| | Gavel: prediction of cleavage sites for mitochondrial | | | | |
| | preseq | | | | |
| | R-2 motif at 30 RRP\|KK | | | | |
| | NUCDISC: discrimination of nuclear localization signals | | | | |
| | pat4: PRRR (4) at 17 | | | | |
| | pat4: RRRP (4) at 18 | | | | |
| | pat4: RRPK (4) at 19 | | | | |
| | pat4: RPKK (4) at 20 | | | | |
| | pat7: PRRRPKK (5) at 17 | | | | |
| | bipartite: KKGTVAPHDQSPRRRPK at 6 | | | | |
| | bipartite: RRPKKGLIKKKMVKREK at 19 | | | | |
| | bipartite: KKGLIKKKMVKREKQKR at 22 | | | | |
| | content of basic residues: 10.3% | | | | |
| | NLS Score: 2.60 | | | | |
| | KDEL: ER retention motif in the C-terminus: none | | | | |
| | ER Membrane Retention Signals: none | | | | |
| | SKL: peroxisomal targeting signal in the C-terminus: | | | | |
| | none | | | | |
| | PTS2: 2nd peroxisomal targeting signal: none | | | | |
| | VAC: possible vacuolar targeting motif: found | | | | |
| | ILPI at 447 | | | | |
| | RNA-binding motif: none | | | | |
| | Actinin-type actin-binding motif: | | | | |
| | type 1: none | | | | |
| | type 2: none | | | | |
| | NMYR: N-myristoylation pattern: none | | | | |
| | Prenylation motif: none | | | | |
| | memYQRL: transport motif from cell surface to Golgi: | | | | |
| | none | | | | |
| | Tyrosines in the tail: none | | | | |
| | Dileucine motif in the tail: none | | | | |
| | checking 63 PROSITE DNA binding motifs: none | | | | |
| | checking 71 PROSITE ribosomal protein motifs: none | | | | |
| | checking 33 PROSITE prokaryotic DNA binding motifs: | | | | |
| | none | | | | |
| | NNCN: Reinhardt's method for Cytoplasmic/Nuclear | | | | |
| | discrimination | | | | |
| | Prediction: cytoplasmic | | | | |
| | Reliability: 94.1 | | | | |
| | COIL: Lupas's algorithm to detect coiled-coil regions | | | | |
| | 30 V | 0.63 | | | |
| | 31 K | 0.71 | | | |
| | 32 R | 0.71 | | | |
| | 33 E | 0.71 | | | |
| | 34 K | 0.71 | | | |
| | 35 Q | 0.71 | | | |
| | 36 K | 0.71 | | | |
| | 37 R | 0.71 | | | |
| | 38 N | 0.71 | | | |
| | 39 M | 0.71 | | | |
| | 40 E | 0.71 | | | |
| | 41 E | 0.71 | | | |
| | 42 L | 0.71 | | | |
| | 43 K | 0.71 | | | |
| | 44 K | 0.71 | | | |
| | 45 E | 0.71 | | | |
| | 46 V | 0.71 | | | |
| | 47 V | 0.71 | | | |
| | 48 M | 0.71 | | | |
| | 49 D | 0.71 | | | |
| | 50 D | 0.71 | | | |
| | 51 H | 0.71 | | | |
| | 52 K | 0.71 | | | |
| | 53 L | 0.71 | | | |
| | 54 T | 0.71 | | | |
| | 55 L | 0.71 | | | |
| | 56 E | 0.71 | | | |
| | 57 E | 0.71 | | | |
| | 58 L | 0.71 | | | |

total: 29 residues
Final Results (k = 9/23):
55.6%: endoplasmic reticulum
11.1%: Golgi
11.1%: vesicles of secretory system
11.1%: nuclear
11.1%: vacuolar
>> prediction for CG161527-01 is end (k = 9)

A search of the NOV14a protein against the Geneseq database, a proprietary database that contains sequences published in patents and patent publication, yielded several homologous proteins shown in Table 14C.

TABLE 14C
Geneseq Results for NOV14a

| | | NOV14a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABP52413 | Human TCH115 protein | 1 . . . 1029 | 1028/1029 (99%) | 0.0 |
| | SEQ ID NO: 24 - Homo | 1 . . . 1029 | 1028/1029 (99%) | |
| | sapiens, 1029 aa. | | | |
| | [WO200261095-A1, | | | |
| | 08 AUG. 2002] | | | |
| ABP52415 | Human TCH115 protein | 1 . . . 1029 | 1027/1029 (99%) | 0.0 |
| | SEQ ID NO: 34 - Homo | 1 . . . 1029 | 1028/1029 (99%) | |
| | sapiens, 1029 aa. | | | |
| | [WO200261095-A1, | | | |
| | 08 AUG. 2002] | | | |
| ABP52412 | Human TCH115 protein | 30 . . . 1029 | 999/1000 (99%) | 0.0 |
| | SEQ ID NO: 1 - Homo | 1 . . . 1000 | 999/1000 (99%) | |
| | sapiens, 1000 aa. | | | |
| | [WO200261095-A1, | | | |
| | 08 AUG. 2002] | | | |
| ABP52414 | Human TCH115 protein | 30 . . . 1029 | 998/1000 (99%) | 0.0 |
| | SEQ ID NO: 33 - Homo | 1 . . . 1000 | 999/1000 (99%) | |
| | sapiens, 1000 aa. | | | |
| | [WO200261095-A1, | | | |
| | 08 AUG. 2002] | | | |
| AAU10501 | Rat (Na, K)-ATPase - Rattus | 29 . . . 1029 | 795/1003 (79%) | 0.0 |
| | rattus, 1212 aa. | 100 . . . 1102 | 886/1003 (88%) | |
| | [US6309874-B1, | | | |
| | 30 OCT. 2001] | | | |

In a BLAST search of public sequence datbases, the NOV14a protein was found to have homology to the proteins shown in the BLASTP data in Table 14D.

TABLE 14D
Public BLASTP Results for NOV14a

| | | NOV14a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q13733 | Sodium/potassium-transporting | 1 . . . 1029 | 1029/1029 (100%) | 0.0 |
| | ATPase alpha-4 chain (EC | 1 . . . 1029 | 1029/1029 (100%) | |
| | 3.6.3.9) (Sodium pump 4) | | | |
| | (Na+/K+ ATPase 4) - Homo | | | |
| | sapiens (Human), 1029 aa. | | | |
| Q9WV27 | Sodium/potassium-transporting | 6 . . . 1029 | 858/1027 (83%) | 0.0 |
| | ATPase alpha-4 chain (EC | 6 . . . 1032 | 947/1027 (91%) | |
| | 3.6.3.9) (Sodium pump 4) | | | |
| | (Na+/K+ ATPase 4) - Mus | | | |
| | musculus (Mouse), 1032 aa. | | | |
| Q64541 | Sodium/potassium-transporting | 7 . . . 1029 | 853/1025 (83%) | 0.0 |
| | ATPase alpha-4 chain (EC | 5 . . . 1028 | 938/1025 (91%) | |
| | 3.6.3.9) (Sodium pump 4) | | | |
| | (Na+/K+ ATPase 4) - Rattus | | | |
| | norvegicus (Rat), 1028 aa. | | | |
| Q9UQ25 | KIAA0778 protein - Homo | 32 . . . 1029 | 804/999 (80%) | 0.0 |
| | sapiens (Human), 1049 aa | 51 . . . 1049 | 902/999 (89%) | |
| | (fragment). | | | |
| P50993 | Sodium/potassium-transporting | 32 . . . 1029 | 804/999 (80%) | 0.0 |
| | ATPase alpha-2 chain | 22 . . . 1020 | 902/999 (89%) | |
| | precursor (EC 3.6.3.9) (Sodium | | | |
| | pump 2) (Na+/K+ ATPase 2) - | | | |
| | Homo sapiens (Human), 1020 | | | |
| | aa. | | | |

PFam analysis predicts that the NOV14a protein contains the domains shown in the Table 14E.

TABLE 14E
Domain Analysis of NOV14a

| | | Identities/ | |
|---|---|---|---|
| | | Similarities | |
| | NOV14a | for the | |
| | Match | Matched | Expect |
| Pfam Domain | Region | Region | Value |
| Cation_ATPase_N | 41 . . . 124 | 41/87 (47%) | 2.1e−34 |
| | | 73/87 (84%) | |
| E1-E2_ATPase | 143 . . . 374 | 102/244 (42%) | 1.2e−113 |
| | | 214/244 (88%) | |
| Hydrolase | 378 . . . 744 | 42/373 (11%) | 1.7e−14 |
| | | 231/373 (62%) | |
| Cation_ATPase_C | 840 . . . 1028 | 72/193 (37%) | 6.3e−92 |
| | | 174/193 (90%) | |

### Example 15

The NOV15 clone was analyzed, and the nucleotide and encoded polypeptide sequences are shown in Table 15A.

TABLE 15A
NOV15 Sequence Analysis

| | |
|---|---|
| SEQ ID NO: 133 | 1714 bp |

| | |
|---|---|
| NOV15a, | GGATGGATGACAGTGACAGAGCTCTAAATTTCTACTAACCAGCTGAGACACAATGGCCAAAAAAGCG |
| CG161579-01 | |
| DNA Sequence | ATTGCTGTGATTGGAGCTGGAATTAGCGGACTGGGGGCCATCAAGTGCTGCCTGGATGAAGATCTGG |
| | AGCCCACCTGCTTTGAAAGAAATGATGATATTGGACATCTCTGGAAATTTCAAAAAAATACTTCAGA |
| | GAAAATGCCTAGTATCTACAAATCTGTGACCATCAATACTTCCAAGGAGATGATGTGCTTCAGTGAC |
| | TTCCCTGTCCCTGATCATTTTCCCAACTACATGCACAACTCCAAACTCATGGACTACTTCGGGATGT |
| | ATGCCACACACTTTGGCCTCCTGAATTACATTCGTTTTAAGACTGAAGTGCAAAGTGTGAGGAAGCA |
| | CCCAGATTTTTCTATCAATGGACAATGGGATGTTGTTGTGGAGACTGAAGAGAAACAAGAGACTTTG |
| | GTCTTTGATGGGGTCTTAGTTTGCAGTGGACACCACACAGATCCCTACTTACCACTTCAGTCCTTCC |
| | CAGGCATTGAGAAATTTGAAGGCTGTTATTTCCATAGTCGGGAATACAAAAGTCCCGAGGACTTTTC |
| | AGGGAAAAGAATCATAGTGATCGGCATTGGAAATTCTGGAGTGGATATTGCGGTGGAGCTCAGTCGT |
| | GTAGCAAAACAGATATTCCTTAGTACTAGACGTGGATCATGGATTTTACACCGTGTTTGGGATAATG |
| | GGTATCCCATGGATAGTTCATTTTTCACTCGGTTCAATAGTTTTCTCCAGAAAATACTAACTACACC |
| | ACAAATAAATAACCAGCTAGAGAAAATAATGAACTCAAGATTTAATCATGCGCACTGTGGCCTGCAG |
| | CCTCAGCACAGGGCTTTAAGTCAGCATCCAACTGTCAGTGATGACCTGCCAAATCACATAATTTCTG |
| | GAAAAGTCCAAGTAAAGCCCAGCGTGAAGGAGTTCACAGAAACAGATGCCATTTTTGAAGACAGCAC |
| | TGTAGAGGAGAATATTGATGTTGTCATCTTTGCTACAGGATACAGTTTTTCTTTTTCTTTCCTTGAT |
| | GGTCTGATCAAGGTTACTAACAATGAAGTATCTCTGTATAAGCTTATGTTCCCTCCTGACCTGGAGA |
| | AGCCAACCTTGGCTGTCATCGGTCTTATCCAACCACTGGGCATCATCTTACCTATTGCAGAGCTCCA |
| | ATCTCGTTGGGCTACACGAGTGTTCAAAGGGCTGATCAAATTACCCTCAGCGGAGAACATGATGGCA |
| | GATATTGCCCAGAGGAAAAGGGCTATGGAAAAAAGATATGTAAAGACACCCCGCCACACAATCCAAG |
| | TGGATCACATTGAGTACATGGATGAGATTGCCATGCCAGCAGGGGTGAAACCCAACCTGCTCTTCCT |
| | CTTTCTCTCAGATCCAAAGCTGGCCATGGAGGTTTTCTTTGGCCCCTGCACCCCATACCAGTACCAC |
| | CTCCATGGGCCCGAGAAATGGGATGGGGCCCGGAGAGCTAACCTGACCCAGAGAGAGAGGATCATCA |
| | AGCCCCTGAGCACTCGCATTACTAGTGAGGACAGCCACCCATCCTCACAGCTCTCTTGGATAAAGAT |
| | GGCCCCAGTGAGCCTGGCATTTCTGGCTGCTGGCTTGGCATACTTTCGATATACTCATTACGGTAAA |
| | TGGAAATAAATGAAAGAACACTGAGGGGGAAAAGCATGG |

| | | |
|---|---|---|
| ORF Start: ATG at 53 | | ORF Stop: TAA at 1682 |
| SEQ ID NO: 134 | 543 aa | MW at 61938.6kD |

| | |
|---|---|
| NOV15a, | MAKKAIAVIGAGISGLGAIKCCLDEDLEPTCFERNDDIGHLWKFQKNTSEKMPSIYKSVTINTSKEM |
| CG161579-01 | |
| Protein Sequence | MCFSDFPVPDHFPNYMHNSKLMDYFGMYATHFGLLNYIRFKTEVQSVRKHPDFSINGQWDVVVETEE |
| | KQETLVFDGVLVCSGHHTDPYLPLQSFPGIEKFEGCYFHSREYKSPEDFSGKRIIVIGIGNSGVDIA |
| | VELSRVAKQIFLSTRRGSWILHRVWDNGYPMDSSFFTRFNSFLQKILTTPQINNQLEKIMNSRFNHA |
| | HCGLQPQHRALSQHPTVSDDLPNHIISGKVQVKPSVKEFTETDAIFEDSTVEENIDVVIFATGYSFS |
| | FSFLDGLIKVTNNEVSLYKLMFPPDLEKPTLAVIGLIQPLGIILPIAELQSRWATRVFKGLIKLPSA |
| | ENMMADIAQRKRAMEKRYVKTPRHTIQVDHIEYMDEIAMPAGVKPNLLFLFLSDPKLAMEVFFGPCT |
| | PYQYHLHGPEKWDGARRANLTQRERIIKPLSTRITSEDSHPSSQLSWIKMAPVSLAFLAAGLAYFRY |
| | THYGKWK |

Further analysis of the NOV15a protein yielded the following properties shown in Table 15B.

TABLE 15B
Protein Sequence Properties NOV15a

| SignalP | | | | |
|---|---|---|---|---|
| analysis: | No Known Signal Sequence Predicted | | | |
| PSORT II | PSG: a new signal peptide prediction method | | | |
| analysis: | N-region: length 4; pos.chg 2; neg.chg 0 | | | |
| | H-region: length 15; peak value 8.96 | | | |
| | PSG score: 4.56 | | | |
| | GvH: von Heijne's method for signal seq. recognition | | | |
| | GvH score (threshold: −2.1): −6.84 | | | |
| | possible cleavage site: between 18 and 19 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 4 | | | |
| | Number of TMS(s) for threshold 0.5: 1 | | | |
| | INTEGRAL Likelihood = −5.89 Transmembrane | | | |
| | 366-382 | | | |
| | PERIPHERAL Likelihood = 0.53 (at 323) | | | |
| | ALOM score: −5.89 (number of TMSs: 1) | | | |
| | MTOP: Prediction of membrane topology (Hartmann et al.) | | | |
| | Center position for calculation: 373 | | | |
| | Charge difference: 3.0 C(2.0) - N(−1.0) | | | |
| | C > N: C-terminal side will be inside | | | |
| | >>>Caution: Inconsistent mtop result with signal | | | |
| | peptide | | | |
| | >>> membrane topology: type 1b (cytoplasmic tail | | | |
| | 366 to 543) | | | |
| | MITDISC: discrimination of mitochondrial targeting seq | | | |
| | R content: | 0 | Hyd Moment(75): | 10.25 |
| | Hyd Moment (95): | 8.17 | G content: | 4 |
| | D/E content: | 1 | S/T content: | 1 |

```
Score: −5.79
Gavel: prediction of cleavage sites for mitochondrial
preseq
cleavage site motif not found
NUCDISC: discrimination of nuclear localization signals
pat4: none
pat7: none
bipartite: none
content of basic residues: 11.0%
MLS Score: −0.47
KDEL: ER retention motif in the C-terminus: none
ER Membrane Retention Signals:
KKXX-like motif in the C-terminus: YGKW
SKL: peroxisomal targeting signal in the C-terminus:
none
PTS2: 2nd peroxisomal targeting signal: none
VAC: possible vacuolar targeting motif: found
ILPI at 378
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: too long tail
Dileucine motif in the tail: found
LL at 449
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 89
COIL: Lupas's algorithm to detect coiled-coil regions
total: 0 residues
Final Results (k = 9/23):
34.8%: nuclear
26.1%: mitochondrial
21.7%: cytoplasmic
  4.3%: vacuolar
  4.3%: vesicles of secretory system
  4.3%: endoplasmic reticulum
  4.3%: peroxisomal
>> prediction for CG161579-01 is nuc (k = 23)
```

A search of the NOV15a protein against the Geneseq database, a proprietary database that contains sequences published in patents and patent publication, yielded several homologous proteins shown in Table 15C.

TABLE 15C
Geneseq Results for NOV15a

| | | | Identities/ | |
|---|---|---|---|---|
| | | | Similarities for | |
| Geneseq | Protein/Organism/Length | NOV15a Residues/ | the Matched | Expect |
| Identifier | [Patent #, Date] | Match Residues | Region | Value |
| AAE21044 | Human drug metabolising | 1 . . . 543 | 540/544 (99%) | 0.0 |
| | enzyme (DME-2) protein - | 1 . . . 544 | 541/544 (99%) | |
| | Homo sapiens, 544 aa. | | | |
| | [WO200212467-A2, | | | |
| | 14 FEB. 2002] | | | |
| AAM40962 | Human polypeptide SEQ ID | 1 . . . 534 | 292/535 (54%) | e−175 |
| | NO 5893 - Homo sapiens, | 16 . . . 544 | 391/535 (72%) | |
| | 550 aa. [WO200153312-A1, | | | |
| | 26 JUL. 2001] | | | |
| AAW49699 | Human flavin-containing | 1 . . . 534 | 291/535 (54%) | e−174 |
| | mono-oxygenase 2 - Homo | 1 . . . 529 | 390/535 (72%) | |
| | sapiens, 535 aa. | | | |
| | [WO9824914-A1, | | | |
| | 11 JUN. 1998] | | | |
| ABG31581 | Human flavin containing | 1 . . . 534 | 288/535 (53%) | e−172 |
| | monooxygenase-2 (FMO2) | 1 . . . 529 | 387/535 (71%) | |
| | variant protein - Homo | | | |
| | sapiens, 535 aa. | | | |
| | [WO200253579-A2, | | | |
| | 11 JUL. 2002] | | | |
| AAR97549 | Human flavin-containing | 4 . . . 531 | 287/529 (54%) | e−169 |
| | monooxygenase - Homo | 3 . . . 529 | 370/529 (69%) | |
| | sapiens, 532 aa. | | | |
| | [EP712932-A2, | | | |
| | 22 MAY 1996] | | | |

In a BLAST search of public sequence datbases, the NOV15a protein was found to have homology to the proteins shown in the BLASTP data in Table 15D.

TABLE 15D
Public BLASTP Results for NOV15a

| | | | Identities/ | |
|---|---|---|---|---|
| Protein | | | Similarities for | |
| Accession | | NOV15a Residues/ | the Matched | Expect |
| Number | Protein/Organism/Length | Match Residues | Portion | Value |
| A46677 | dimethylaniline | 1 . . . 534 | 337/534 (63%) | 0.0 |
| | monooxygenase | 1 . . . 533 | 420/534 (78%) | |
| | (N-oxide-forming) (EC | | | |
| | 1.14.13.8) 1C1- rabbit, 533 | | | |
| | aa. | | | |
| Q8K4C0 | Flavin-containing | 1 . . . 534 | 338/534 (63%) | 0.0 |
| | monooxygenase 5 (EC | 1 . . . 533 | 417/534 (77%) | |
| | 1.14.13.8) - Rattus norvegicus | | | |
| | (Rat), 533 aa. | | | |
| Q04799 | Dimethylaniline | 2 . . . 534 | 336/533 (63%) | 0.0 |
| | monooxygenase [N-oxide | 1 . . . 532 | 419/533 (78%) | |
| | forming] 5 (EC 1.14.13.8) | | | |
| | (Hepatic flavin-containing | | | |
| | monooxygenase 5) (FMO 5) | | | |
| | (Dimethylaniline oxidase 5) | | | |
| | (FMO 1C1) (FMO form 3) - | | | |
| | Oryctolagus cuniculus | | | |
| | (Rabbit), 532 aa. | | | |
| S71618 | dimethylaniline | 1 . . . 534 | 330/534 (61%) | 0.0 |
| | monooxygenase | 1 . . . 533 | 418/534 (77%) | |
| | (N-oxide-forming) (EC | | | |
| | 1.14.13.8) FMO5- human, | | | |
| | 533 aa. | | | |
| Q8R1W6 | Flavin containing | 1 . . . 534 | 335/534 (62%) | 0.0 |
| | monooxygenase 5 - Mus | 1 . . . 533 | 414/534 (76%) | |
| | musculus (Mouse), 533 aa. | | | |

PFam analysis predicts that the NOV15a protein contains the domains shown in the Table 15E.

TABLE 15E
Domain Analysis of NOV15a

| | | Identities/ | |
|---|---|---|---|
| | | Similarities | |
| | NOV15a | for the | |
| Pfam | Match | Matched | Expect |
| Domain | Region | Region | Value |
| pyr_redox | 5 . . . 360 | 48/438 (11%) | 0.0092 |
| | | 223/438 (51%) | |
| FMO-like | 3 . . . 534 | 327/536 (61%) | 0 |
| | | 427/536 (80%) | |

### Example 16

The NOV16 clone was analyzed, and the nucleotide and encoded polypeptide sequences are shown in Table 16A.

TABLE 16A
NOV16 Sequence Analysis

| | |
|---|---|
| SEQ ID NO: 135 | 345 bp |

| | |
|---|---|
| NOV16a, | CATCATCCAGGAAAGACGTTGGTTTTGAGGTTGACATACCTATAAAGGACAGTAACTACCATGGCTT |
| CG161650-01 | |
| DNA Sequence | CCACCGCTTTGGCAAAACCTCAGATGTGTGGTCTTCTGGCCAAATGTCTGCAATTTCATATTGTTGG |
| | AGCCTTTATTGTATCCCTGGGGGTTGCAGCTGTCTGTAAGATTGCTGTGGCTGAACCAAGAAAGAAG |
| | ACATATGCAGATTTCTACAGAAATTATGATTCCGTGAAAGATTTGGAGGAGATGGGGAAGGCTGGTA |
| | TCTTTCAGAATACAAAGTGATTTTGGAATGCAAAGGATTTCTTTGGGTTGAATTACCTAGAAGTTTG |
| | TCACTTACCT |

| | | |
|---|---|---|
| ORF Start: ATG at 61 | | ORF Stop: TGA at 286 |
| SEQ ID NO: 136 | 75 aa | MW at 8171.6kD |

| | |
|---|---|
| NOV16a, | MASTALAKPQMCGLLAKCLQFHIVGAFIVSLGVAAVCKIAVAEPRKKTYADFYRNYDSVKDLEEMGK |
| CG161650-01 | |
| Protein Sequence | AGIFQNTK |

Further analysis of the NOV16a protein yielded the following properties shown in Table 16B.

TABLE 16B
Protein Sequence Properties NOV16a

| SignalP analysis: | Cleavage site between residues 43 and 44 | |
|---|---|---|
| PSORT II analysis: | PSG: | a new signal peptide prediction |
| | | method |
| | | N-region: length 8; |
| | | pos.chg 1; neg.chg 0 |
| | | H-region: length 8; peak value 4.86 |
| | | PSG score: 0.46 |
| | GvH: | von Heijne's method for |
| | | signal seq. recognition |
| | | GvH score (threshold: −2.1): −4.84 |
| | | possible cleavage site: |
| | | between 26 and 27 |
| | >>>Seems to have no N-terminal signal peptide | |
| | ALOM: | Klein et al's method for |
| | | TM region allocation |
| | | Init position for calculation: 1 |
| | | Tentative number of TMS(s) |
| | | for the threshold 0.5: 1 |
| | | Number of TMS(s) |
| | | for threshold 0.5: 1 |
| | | INTEGRAL Likelihood = −6.58 |
| | | Transmenbrane 23-39 |
| | | PERIPHERAL |
| | | Likelihood = 7.11 (at 5) |
| | | ALOM score: |
| | | −6.58 (number of TMSs: 1) |
| | MTOP: | Prediction of membrane |
| | | topology (Hartmann et al.) |
| | | Center position for calculation: 30 |
| | | Charge difference: -0.5 |
| | | C(2.0) − N(2.5) |
| | | N >= C: N-terminal |
| | | side will be inside |
| | >>>membrane topology: type 2 | |
| | (cytoplasmic tail 1 to 23) | |
| | MITDISC: | discrimination of mitochondrial |
| | | targeting seq |
| | | R content: 0 Hyd Moment(75): 7.56 |
| | | Hyd Moment(95): 5.39 G content: 3 |
| | | D/E content: 1 S/T content: 3 |
| | | Score: - 5.40 |
| | Gavel: | prediction of cleavage sites for |
| | | mitochondrial preseq |
| | | cleavage site motif not found |
| | NUCDISC: | discrimination of nuclear |
| | | localization signals |
| | | pat4: PRKK (4) at 44 |
| | | pat7: PRKKTYA (5) at 44 |
| | | bipartite: none |
| | | content of basic residues: 13.3% |
| | | NLS Score: 0.21 |
| | KDEL: | ER retention motif in |
| | | the C-terminus: none |
| | ER Membrane Retention Signals: none | |
| | SKL: | peroxisomal targeting signal |
| | | in the C-terminus: none |
| | PTS2: 2nd peroxisomal targeting signal: none | |
| | VAC: possible vacuolar targeting motif: none | |
| | RNA-binding motif: none | |
| | Actinin-type actin-binding motif: | |
| | | type 1: none |
| | | type 2: none |
| | NMYR: | N-myristoylation pattern : none |
| | Prenylation motif: none | |
| | memYQRL: transport motif from cell | |
| | surface to Golgi: none | |
| | Tyrosines in the tail: none | |
| | Dileucine motif in the tail: found | |
| | | LL at 14 |
| | checking 63 PROSITE DNA binding motifs: none | |
| | checking 71 PROSITE ribosomal protein | |
| | motifs: none | |
| | checking 33 PROSITE prokaryotic | |
| | DNA binding motifs: none | |
| | NNCN: | Reinhardt's method for Cytoplasmic/ |
| | | Nuclear discrimination |
| | | Prediction: cytoplasmic |
| | | Reliability: 94.1 |
| | COIL: | Lupas's algorithm to detect |
| | | coiled-coil regions |
| | | total: 0 residues |
| | Final Results (k = 9/23) | |
| | | 30.4 %: cytoplasmic |
| | | 30.4 %: mitochondrial |
| | | 13.0 %: Golgi |
| | | 8.7 %: endoplasmic reticulum |
| | | 4.3 %: extracellular, |
| | | including cell wall |
| | | 4.3 %: vacuolar |
| | | 4.3 %: vesicles of secretory system |
| | | 4.3 %: nuclear |

>>prediction for CG161650-01 is cyt (k = 23)

A search of the NOV16a protein against the Geneseq database, a proprietary database that contains sequences published in patents and patent publication, yielded several homologous proteins shown in Table 16C.

TABLE 16C
Geneseq Results for NOV16a

| | | NOV16a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM79710 | Human protein SEQ ID NO | 6 . . . 75 | 47/70 (67%) | 1e−20 |
| | 3356 - Homo sapiens, 83 aa. | 15 . . . 83 | 56/70 (79%) | |
| | [WO200157190-A2, 09-AUG-2001] | | | |
| ABP62939 | Human polypeptide SEQ ID | 1 . . . 75 | 47/75 (62%) | 1e−19 |
| | NO 376 - Homo sapiens, 75 | 1 . . . 75 | 57/75 (75%) | |
| | aa. [WO200218424-A2, 07-MAR-2002] | | | |
| AAB56523 | Human prostate cancer | 1 . . . 75 | 47/75 (62%) | 1e−19 |
| | antigen protein sequence SEQ | 19 . . . 93 | 57/75 (75%) | |
| | ID NO: 1101 - Homo sapiens, | | | |
| | 93 aa. [WO200055174-A1, | | | |
| | 21-SEP-2000] | | | |
| AAM23875 | Human EST encoded protein | 26 . . . 75 | 35/50 (70%) | 9e−14 |
| | SEQ ID NO: 1400 - Homo | 4 . . . 53 | 41/50 (82%) | |
| | sapiens, 53 aa. | | | |
| | [WO200154477-A2, 02-AUG-2001] | | | |
| AAM78726 | Human protein SEQ ID NO | 1 . . . 33 | 22/33 (66%) | 8e−06 |
| | 1388 - Homo sapiens, 74 aa. | 12 . . . 44 | 27/33 (81%) | |
| | [WO200157190-A2, 09-AUG-2001] | | | |

In a BLAST search of public sequence datbases, the NOV16a protein was found to have homology to the proteins shown in the BLASTP data in Table 16D.

TABLE 16D.
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P04038 | Cytochrome c oxidase | 3 . . . 75 | 56/73 (76%) | 2e−24 |
| | polypeptide VIC (EC 1.9.3.1) | 1 . . . 73 | 61/73 (82%) | |
| | (STA) - Bos taurus (Bovine), 73 aa. | | | |
| S00114 | cytochrome-c oxidase (EC | 6 . . . 75 | 51/70 (72%) | 8e−23 |
| | 1.9.3.1) chain VIc [validated]- | 7 . . . 76 | 58/70(82%) | |
| | rat, 76 aa. | | | |
| P11951 | Cytochrome c oxidase | 6 . . . 75 | 51/70(72%) | 8e−23 |
| | polypeptide VIC-2 (EC | 6 . . . 75 | 58/70 (82%) | |
| | 1.9.3.1) - Mus musculus | | | |
| | (Mouse), and, 75 aa. | | | |
| Q9CPQ1 | Adult male hippocampus | 6 . . . 75 | 52/70 (74%) | 1e−22 |
| | cDNA, RIKEN full-length | 7 . . . 76 | 57/70 (81%) | |
| | enriched library, clone: 2900001B12, | | | |
| | full insert sequence (11 days | | | |
| | embryo cDNA, RIKEN | | | |
| | full-length enriched library, | | | |
| | clone: 2700093G08, full insert | | | |
| | sequence) (Cytochrome c | | | |
| | oxidase, subunit VIc) - Mus | | | |
| | musculus (Mouse), 76 aa. | | | |
| CAB25169 | RAT CYTOCHROME C | 6 . . . 75 | 51/70 (72%) | 4e−22 |
| | OXIDASE SUBUNIT VIC | 7 . . . 76 | 55/70 (77%) | |
| | PROCESSED | | | |
| | PSEUDOGENE, COMPLETE | | | |
| | CDS - Rattus norvegicus (Rat), 76 aa. | | | |

PFam analysis predicts that the NOV16a protein contains the domains shown in the Table 16E.

TABLE 16E
Domain Analysis of NOV16a

| | | Identities/ | |
|---|---|---|---|
| | NOV16a | Similarities for | |
| Pfam Domain | Match Region | the Matched Region | Expect Value |
| COX6C | 1 . . . 75 | 49/75 (65%) | 4e−44 |
| | | 66/75 (88%) | |

### Example 17

The NOV17 clone was analyzed, and the nucleotide and encoded polypeptide sequences are shown in Table 17A.

TABLE 17A
NOV17 Sequence Analysis

| | |
|---|---|
| SEQ ID NO: 137 | 12258 bp |

| | |
|---|---|
| NOV17a, | ATGCAAAGACTGAAGGGAGAGAAGGAAGCCAAGCGGGCTCTTTTGGATGCGAGGCATAACTACTTAT |
| CG161733-01 | |
| DNA Sequence | TTGCAATTGTGGCTTCCTGTTTGGACCTGAACAAAACCGAAGTGGAGGATGCCATTCTTGAAGGGAA |
| | TCAGATTGAAAGAATTGATCAACTTTTTGCTGTTGGAGGTCTCCGACACCTCATGTTTTACTATCAA |
| | GATGTGGAGGAAGCAGAAACAGGACAACTTGGCTCTCTAGGAGGGGTAAATCTTGTTTCTGGAAAGA |
| | TTAAAAAACCTAAGGTGTTCGTGACCGAGGGAAACGATGTGGCTCTTACTGGGGTATGTGTGTTCTT |
| | CATCAGGACTGACCCTTCCAAAGCCATCACCCCTGACAACATCCACCAGGAGGTGAGTTTTAACATG |
| | TTAGATGCGGCAGATGGAGGCCTGCTCAACAGTGTGAGACGTTTGCTGTCGGACATCTTCATTCCTG |
| | CTCTCAGAGCCACGAGCCATGGCTGGGGCGAGCTCGAGGGCCTTCAGGACGCAGCTAACATTCGCCA |
| | GGAGTTCTTGAGCTCCCTGGAAGGCTTTGTGAACGTCCTGTCGGGTGCACAGGAGAGTCTGAAGGAG |
| | AAGGTGAACCTTCGAAAGTGTGACATACTTGAACTGAAAACCCTAAAGGAACCTACGGACTACTTGA |
| | CTCTAGCAAATAACCCTGAGACTTTGGGAAAAATAGAGGATTGCATGAAAGTATGGATCAAACAGAC |
| | AGAACAGGTTCTTGCTGAAAACAATCAGCTGCTGAAGGAAGCGGATGACGTTGGGCCACGAGCGGAG |
| | CTGGAGCACTGGAAAAAAAGACTCTCCAAGTTTAACTACCTTTTGGAACAATTGAAAAGCCCGGATG |
| | TGAAGGCTGTGCTGGCAGTGCTTGCGGCGGCCAAGTCGAAACTGCTGAAGACTTGGCGGGAGATGGA |
| | TATTCGAATCACTGATGCAACTAATGAAGCAAAGGACAATGTGAAATACTTGTATACACTTGAAAAA |
| | TGTTGTGACCCTTTGTACAGCAGTGATCCCGTGTCCATGATGGATGCTATTCCTACACTTATAAATG |
| | CAATTAAAATGATCTATAGTATCTCTCATTACTATAATACCTCTGAGAAGATCACATCTCTGTTTGT |
| | AAAGGTAACAAATCAGATTATATCTGCATGTAAAGCCTATATTACCAATAATGGAACCGCTTCCATC |
| | TGGAACCAGCCACAGGATGTTGTTGAAGAAAAAATACTATCTGCGATTTTTGATTTTAAGGAATACC |
| | AGCTCTGCTTTCACAAGACAAAACAAAAGCTTAAACAAAATCCAAATGCAAAACAATTTGATTTTAG |
| | CGAGATGTATATTTTTGGAAAATTCGAAACTTTTCACCGACGCCTTGCCAAGATAATAGACATCTTT |
| | ACAACCCTCAAGACGTATTCAGTCCTGCAAGATTCCACAATTGAAGGGCTGGAAGACATGGCCACTA |
| | AATACCAGGTATTGTACTTTAAAATAAAGAAAAAGGAATACAATTTCCTAGACCAGCGGAAAATGGA |
| | TTTTGACCAAGATTACGAAGAGTTTTGCAAGCAGACTAATGACCTTCATGTAGAGTTGCGGAAGTTC |
| | ATGGATGTTACATTTGCAAAGATTCAAAACACAAATCAAGCTCTAAGAATGTTGAAGAAATTTGAAA |
| | GGGCACAAATACTACATTTTAAACTTGGTATTGATGACAAATATCAACTTATCCTTGAGAACTATGG |
| | GGCTGACATTGATATGATTTCAAAGCTGTATACAAAGCAGAAATACGATCCTCCTCTGGCTCGAAAC |
| | CAGCCTCCCATCGCTGGAAAGATTTTGTGGGCCCGCCAGCTCTTCCATAGGATTCAGCAGCCCATGC |
| | AGCTTTTCCAGCAGCACCCAGCTGTGCTAAGCACGGCAGAAGCCAAACCTATAATTCGCAGTTACAA |
| | CAGGATGGCCAAGGTCCTCCTGGAGTTTGAGGTCCTCTTCCACAGGGCGTGGCTTCGGCAAGTGAGT |
| | GAAATTCATGTAGGTCTTGAGGCTTCATTATTGGTGAAGGCTCCAGGCACAGGGGAATTGTTTGTAA |
| | ACTTTGACCCTCAGATATTAATCTTATTTAGAGAAACAGAGTGCATGGCCCAGATGGGTCTGGAAGT |
| | CTCTCCACTGGCAACTTCCCTCTTCCAGTTTGAAGGAGGTGCAAAGGCCCTGAGGCTCAGGACCAGA |
| | AAGATGCTAGCTGAATATCAGAGAGTGAAGTCAAAAATACCTGCTGCCATTGAGCAATTGATTGTCC |
| | CTCACTTGGCCAAAGTGGATGAAGCTCTCCAACCTGGCTTGGCTGCACTGACCTGGACATCACTGAA |
| | TATTGAGGCTTATTTAGAAAACACTTTTGCAAAGATCAAGGACCTGGAGTTGCTGCTTGACAGGGTC |
| | AATGATTTGATTGAGTTCCGCATTGATGCCATTCTAGAAGAAATGAGCAGCACGCCTCTTTGTCAGC |
| | TTCCCCAGGAGGAGATGACAAAGGTTGAGGAAATGGTGGAGCCCCATGCTGATTATTCAAGGAATGG |
| | TGCACAAATACTACATTTTAAAAGCTCATTAGTGGAGGAGGCAGTCAATGAGCTTGTAAATATGTTG |
| | CTGGATGTGGAAGTTTTAAAAATATCCAATGAGAATAGTGTTAATTACAAAAATGAAAGTTCAGCAA |
| | AAAGAGAAGAAGGAAATTTTGACACCTTGACATCATCTATTAATGCCAGGGCCAATGCCCTGCTTTT |
| | GACGACAGTCACGAGGAAAAAGAAAGAAACTGAGATGTTAGGGGAAGAAGCCCGCGAGTTACTCTCT |
| | CATTTCAACCATCAGAACATGGATGCTCTTCTGAAAGTTACAAGGAATACACTAGAGGCCATTCGCA |
| | AACGTATTCATTCCTCTCACACAATTAACTTCCGGGGTAATAATCTTGTGCCCATTTTCCGGGCAAG |
| | CGTCACTCTGGCCATTCCCAACATCGTCATGGCCCCTGCCCTGGAAGATGTACAGCAGACCCTGAAC |
| | AAAGCCGTGGAGTGCATCATCAGTGTCCCTAAGGGGGTCAGACAGTGGAGCAAGATACAAGAAAGAA |
| | AAATGGCTGCTTTGCAGAGTAATGAAGACAGTGATTCTGATGTTGAAATGGGAGAAAATGAACTTCA |
| | AGATACCTTGGAGATAGCATCTGTAAATTTACCCATTCCCGTGCAAACCAAGAACTATTATAAGAAT |
| | GTTTCTGAAAACAAAGAGATTGTAAAATTAGTTTCTGTGCTTAGCACAATTATCAACTCCACCAAAA |
| | AGGTATGTCAAGAGGGTCTGGATTGCTTCAAACGCTACAATCACATTTGGCAAAAGGGAAAAGAAGA |
| | AGCCATTAAGACATTTATTACACAGAGCCCCTTGCTTTCTGAATTTGAGTCCCAGATTCTCTATTTC |
| | CAAAACCTAGAGCAGGAAATTAATGCTGAGCCTGAATATGTCTGTGTGGGTTCCATTGCTCTGTACA |
| | CAGCTGACTTGAAGTTCGCCCTGACTGCTGAGACAAAGGCCTGGATGGTTGTCATTGGACGCCACTG |
| | TAACAAAAAATACCGGAGTGAGATGGAAAACATTTTTATGCTTATTGAAGAATTCAATAAGAAACTA |
| | AATCGTCCAATTAAGGACCTAGATGATATTCGGATTGCAATGGCAGCGCTGAAAGAAATAAGGGAGG |
| | AGCAAATCTCCATTGACTTTCAAGTAGGACCTATTGAGGAATCTTATGCCCTGCTTAACAGATATGG |
| | ACTTCTGATAGCAAGGGAAGAGATAGACAAAGTTGATACACTGCACTATGCTTGGGAGAAGCTGCTG |
| | GCACGTGCTGGCGAAGTCCAGAATAAATTAGTCTCACTGCAGCCCAGTTTCAAGAAAGAGCTTATTA |
| | GTGCTGTGGAGGTATTCCTCCAAGATTGTCACCAGTTTTATCTGGACTATGATTTGGTATGTGTTCA |
| | GAATGGTCCAATGGCTAGCGGCTTGAAGCCCCAGGAAGCCAGTGACAGGCTTATCATGTTTCAGGTA |
| | ATCTTTGATAATATCTATCGGAAATACATCACATATACTGGAGGAGAGGAGCTTTTTGGCCTGCCAG |
| | CTACACAGTATCCTCAGCTTCTTGAAATAAAGAAGCAACTAAATCTTCTACAGAAAATATATACTCT |
| | GTACAACAGTGTCATAGAAACTGTAAATAGCTATTATGATATTCTTTGGTCAGAGGTGAATATTGAA |
| | AAAATTAACAATGAACTCTTAGAATTCCAGAACAGGTGTCGAAAGCTTCCCCGGGCCTTGAAGGACT |
| | GGCAGGCTTTTTTGGACCTGAAGAAGATCATTGATGATTTCAGCGAGTGTTGCCCGCTGCTGGAATA |
| | CATGGCCAGTAAAGCCATGATGGAGCGGCACTGGGAAAGGATAACCACCCTCACCGGGCACAGTCTG |
| | GATGTGGGGAATGAAAGCTTTAAGTTAAGAAATATCATGGAGGCACCTCTTCTGAAATATAAAGAGG |
| | AAATAGAGGACATCTGTATCAGTGCGGTGAAAGAGAGAGACATTGAGCAAAAGCTGAAGCAAGTGAT |
| | TAATGAATGGGACAATAAAACATTCACCTTCGGCAGCTTTAAAACCCGTGGAGAGCTCCTCTTGAGA |
| | GGAGACAGTACCTCGGAAATCATCGCCAACATGGAGGACAGCTTGATGTTGCTGGGATCCCTACTGA |
| | GCAACAGGTACAATATGCCATTCAAAGCCCAGATTCAAAAATGGGTGCAGTACCTTTCCAACTCAAC |
| | AGACATCATCGAGAGCTGGATGACGGTGCAAAACCTGTGGATTTATTTAGAAGCTGTCTTTGTGGGA |
| | GGAGACATTGCCAAGCAGCTGCCCAAGGAAGCCAAGCGGTTTTCTAACATAGATAAATCTTGGGTGA |
| | AGATCATGACTCGGGCACATGAAGTGCCCAGTGTAGTCCAGTGCTGTGTTGGAGATGAGACCCTGGG |
| | GCAGCTGTTACCACACTTGCTGGACCAGTTGGAAATATGCCAGAAATCCCTTACTGGGTACTTGGAG |
| | AAAAAACGACTGTGCTTTCCTCGGTTTTTCTTCGTCTCAGATCCTGCCCTTCTAGAGATTCTGGGGC |
| | AGGCGTCGGACTCCCACACTATACAGGCCCATTTGCTGAATGTGTTTGACAACATTAAATCTGTCAA |
| | GTTCCACGAAAAGGTTATCTATGATCGAATTCTGTCAATTTCCTCTCAAGAGGGTGAGACGATTGAA |
| | TTGGATAAACCTGTCATGGCAGAGGGCAATGTGGAAGTTTGGCTTAATTCTCTTTTGGAAGAATCTC |
| | AGTCCTCATTGCATCTTGTGATTCGCCAGGCAGCCGCAAATATTCAAGAAACAGGTTTCCAACTAAC |
| | TGAATTTCTTTCATCCTTCCCTGCTCAGGTCGGATTATTAGGAATTCAGATGATATGGACACGGGAT |
| | TCAGAAGAAGCCCTTAGAAATGCCAAGTTTGATAAAAAAATCATGCAGAAAACTAATCAGGCTTTCC |
| | TGGAGCTACTCAATACATTGATAGACGTCACCACGAGGGATCTGAGTTCCACGGAACGAGTGAAATA |
| | CGAGACTCTGATTACTATTCATGTGCACCAAAGGGATATCTTTGATGACCTGGTACATATGCATATC |
| | AAGAGTCCCATGGACTTTGAGTGGCTGAAACAGTGCAGATTTTACTTTAACGAAGATTCTGACAAGA |
| | TGATGATTCACATCACAGATGTGGCGTTCATATACCAGAATGAATTTTTAGGCTGCACTGACAGGCT |
| | TGTAATAACTCCACTTACAGACAGATGTTACATCACGCTGGCTCAAGCTCTGGGAATGAGCATGGGG |
| | GGAGCCCCTGCTGGACCTGCAGGCACAGGCAAAACAGAAACCACTAAAGACATGGGACGATGCCTCG |
| | GGAAATACGTCGTGGTTTTCAATTGTTCAGACCAGATGGATTTCCGAGGACTTGGACGGATTTTTAA |
| | GGGACTGGCACAGTCTGGATCCTGGGGTTGTTTTGATGAATTTAACCGTATTGATCTACCAGTTCTC |
| | TCGGTTGCAGCCCAGCAAATTTCCATTATTCTGACATGTAAAAAGGAGCACAAAAAGTCTTTTATCT |
| | TTACTGATGGAGATAATGTGACTATGAACCCTGAATTTGGGCTTTTCTTAACCATGAATCCTGGCTA |
| | TGCCGGACGGCAGGAACTCCCTGAAAACTTGAAGATTAATTTCCGCTCAGTGGCCATGATGGTGCCT |
| | GACCGTCAGATTATCATAAGGGTGAAGTTGGCTAGTTGTGGCTTCATTGACAACGTTGTTTTGGCCA |
| | GGAAGTTTTTCACGCTCTACAAACTGTGTGAGGAGCAGCTTTCTAAGCAGGTTCATTATGACTTTGG |
| | CCTGCGTAACATTCTGTCAGTTCTTCGGACCTTGGGAGCAGCAAAAAGAGCCAATCCAATGGATACG |
| | GAGTCCACGATTGTCATGCGTGTACTACGGGACATGAATCTTTCTAAACTGGTAGATGAGGATGAAC |
| | CCTTGTTTTTGAGTTTGATTGAAGATCTCTTTCCAAATATTCTTCTGGACAAGGCAGGTTACCCTGA |
| | ACTGGAAGCAGCAATTAGTAGACAGGTTGAAGAAGCTGGTTTAATCAACCATCCTCCTTGGAAACTG |
| | AAGGTCATCCAGCTATTCGAAACGCAGAGAGTGCGACATGGGATGATGACTCTGGGGCCCAGTGGGG |
| | CTGGGAAGACCACCTGCATCCACACCTTGATGAGAGCCATGACAGATTGTGGAAAACCACATCGGGA |
| | AATGAGGATGAATCCCAAAGCGATTACTGCCCCACAGATGTTTGGTCGGCTGGACGTTGCCACAAAT |
| | GACTGGACTGATGGGATATTTTCTACGCTTTGGAGGAAAACATTAAGAGCAAAGAAAGGTGAACATA |
| | TCTGGATAATTCTTGATGGTCCAGTAGATGCCATCTGGATTGAAAATCTGAATTCTGTTTTGGATGA |
| | TAACAAAACTCTAACCCTTGCCAATGGTGATCGGATTCCCATGGCTCCAAACTGCAAGATCATTTTC |
| | GAGCCTCATAACATTGACAATGCTTCTCCTGCCACCGTCTCAAGAAATGGAATGGTTTTCATGAGCT |
| | CTTCTATCCTTGATTGGAGTCCTATTCTTCAGGGTTTTCTTAAGAAACGCTCACCTCAAGAAGCAGA |
| | AATTCTTCGTCAGCTGTACACCGAGTCTTTCCCAGACTTGTATCGCTTCTGTATCCAGAACTTAGAA |
| | TACAAGATGGAGGTGCTGGAGGCCTTTGTCATCACACAGAGCATTAACATGCTTCAAGGCCTGATTC |
| | CTCTGAAGGAGCAAGGCGGGGAGGTGAGCCAGGCTCACCTGGGGCGGCTGTTCGTGTTCGCGCTGCT |
| | GTGGAGCGCGGGGGCGGCGCTGGAGCTGGACGGACGGCGCCGCCTGGAGCTCTGGCTGCGCTCTCGG |
| | CCCACAGGGACGCTGGAGCTGCCGCCGCCAGCGGGGCCCGGGGACACCGCCTTCGACTACTATGTGG |
| | CGCCCGATGGTACATGGACGCACTGGAACACGCGTACCCAGGAATACCTGTATCCGTCTGATACCAC |
| | CCCAGAGTATGGTTCTATTCTGGTGCCAAATGTTGACAATGTGAGGACTGACTTTCTAATTCAAACC |
| | ATTGCTAAACAGGGCAAGGCTGTGCTATTAATTGGTGAACAAGGAACAGCCAAAACAGTAATAATTA |
| | AAGGATTTATGTCAAAATATGATCCTGAATGTCACATGATCAAGAGTCTGAATTTTTCTTCTGCAAC |
| | CACCCCACTGATGTTCCAGAGGACGATAGAGAGCTATGTGGATAAACGAATGGGTACAACATATGGC |
| | CCTCCTGCGGGAAAGAAGATGACTGTTTTTATTGATGATGTGAATATGCCAATAATCAATGAGTGGG |
| | GAGATCAGGTTACGAATGAGATAGTGCGACAGCTGATGGAACAAAATGGATTCTATAATCTAGAGAA |
| | GCCTGGGGAGTTCACCAGCATCGTGGACATCCAGTTTTTGGCAGCCATGATCCATCCTGGTGGTGGA |
| | CGCAATGACATACCCCAAAGACTCAAGAGGCAGTTCTCTATATTTAATTGCACGTTGCCCTCTGAAG |
| | CTTCTGTGGACAAGATCTTTGGTGTGATTGGGGTAGGCCACTACTGTACTCAGAGGGGTTTCTCAGA |
| | AGAAGTGAGAGATTCTGTGACAAAATTGGTGCCTCTGACACGCCGACTATGGCAGATGACCAAGATT |
| | AAAATGCTTCCTACCCCTGCAAAATTCCATTATGTGTTTAACCTACGAGATCTTTCTCGGGTCTGGC |
| | AGGGAATGCTGAACACTACTTCAGAGCTGTTAAAGCTGTGGAAGCATGAGTGTAAACGTGTTATAGC |
| | TGACCGTTTCACAGTGTCCAGTGATGTGACCTGGTTTGATAAGGCTTTAGTAAGTTTGGTAGAGGAG |
| | GAGTTTGGTGAAGAGAAAAAACTCTTGGTGGATTGTGGAATTGACACATATTTTGTGGATTTCTTGA |
| | GAGATGCACCTGAAGCTGCAGGTGGTGAAACATCTGAAGAGGCTGATGCTGAAACACCTAAAATTTA |
| | TGAGCCAATTGAATCTTTTAGTCACCTAAAAGAGCGTCTGAATATGTTCCTGCAGCTCTATAATGAG |
| | AGCATCCGTGGCGCCGGCATGGACATGGTGTTCTTTGCAGATGCCATGGTTCACTTAGTCAAGATCT |
| | CTCGTGTCATTCGTACTCCTCAGGGAAATGCCCTCCTGGTCGGGGTGGGCGGATCAGGAAAGCAGAG |
| | CCTGACGAGGTTGGCTTCATTCATTGCTGGCTACGTTTCCTTCCAGATCACTCTGACGAGATCCTAC |
| | AACACATCAAATCTGATGGAAGATCTGAAGGTTTTGTATCGAACAGCTGGTCAGCAAGGCAAAGGAA |
| | TCACTTTTATTTTCACAGACAATGAGATTAAAGATGAGTCATTTTTGGAATATATGAACAATGTTTT |
| | ATCATCAGGTGAGGTATCTAACCTATTTGCTCGAGATGAAATTGATGAAATTAATAGCGACCTGGCA |
| | TCAGTCATGAAAAAAGAATTCCCCAGGTGCCTTCCTACCAATGAGAACCTGCACGACTACTTCATGA |
| | GTCGGGTCCGACAGAACCTTCATATTGTGCTCTGCTTCTCGCCAGTGGGGGAGAAATTTCGAAACAG |
| | AGCTTTGAAGTTCCCTGCCCTAATTTCAGGATGCACAATTGACTGGTTCAGCCGATGGCCCAAAGAT |
| | GCTTTAGTTGCTGTGTCTGAACACTTCCTCACTTCCTATGATATTGACTGCAGTTTGGAAATCAAGA |
| | AGGAGGTGGTCCAATGCATGGGCTCCTTCCAGGATGGGGTGGCTGAGAAGTGTGTTGATTATTTTCA |
| | GAGATTCCGACGTTCTACCCACGTGACGCCCAAATCATACCTCTCCTTTATTCAGGGCTATAAGTTC |
| | ATATATGGAGAAAAGCATGTGGAGGTGCGGACCCTGGCCAACAGAATGAATACTGGATTGGAAAAGC |
| | TCAAAGAAGCTTCAGAGTCTGTTGCAGCCTTGAGTAAAGAACTGGAAGCGAAAGAAAAGGAGCTACA |
| | AGTGGCCAACGATAAAGCCGACATGGTCTTAAAAGAAGTGACAATGAAAGCACAGGCTGCTGAAAAG |
| | GTCAAGGCTGAGGTACAGAAGGTGAAGGACAGGGCCCAGGCCATTGTGGACAGCATCTCTAAAGACA |
| | AAGCCATTGCTGAAGAAAAACTGGAAGCAGCAAAACCAGCTTTAGAAGAGGCAGAAGCTGCATTGAC |
| | CATCAGGCCTTCGGACATCGCCACTGTTCGCACGTTGGGCCGCCCCCCTCACCTCATCATGCGGATC |
| | ATGGATTGCGTACTGCTGCTGTTTCAAAGGAAAGTCAGTGCTGTGAAAATTGACCTGGAAAAAAGCT |
| | GTACCATGCCCTCCTGGCAGGAATCCTTAAAATTGATGACTGCAGGGAACTTTTTACAGAACTTACA |
| | GCAATTCCCAAAAGACACAATCAATGAAGAGGTGATAGAATTTTTGAGTCCTTACTTTGAAATGCCT |
| | GACTATAACATCGAAACTGCTAAACGCGTATGTGGAAATGTAGCTGGTCTTTGTTCCTGGACGAAAG |
| | CTATGGCTTCCTTCTTTTCTATAAACAAAGAAGTACTGCCTCTGAAGGCCAACTTGGTGGTGCAAGA |
| | GAATCGCCATCTCCTGGCCATGCAGGATCTGCAGAAAGCCCAGGCCGAGTTGGATGACAAGCAGGCG |
| | GAACTTGACGTGGTGCAGGCTGAGTATGAACAGGCCATGACTGAAAAGTTGCTTGAAGATGCAGAGC |
| | GATGCAGACACAAGATGCAGACAGCTTCCACGCTCATCAGTGGCTTGGCAGGTGAAAAAGAAAGATG |
| | GACAGAGCAAAGCCAAGAGTTTGCTGCACAAACTAAAAGACTTGTAGGTGATGTACTGTTGGCTACA |
| | GCTTTTCTATCTTATTCTGGTCCATTTAACCAAGAGTTTCGTGATCTTCTGTTAAATGACTGGCGGA |
| | AGGAAATGAAAGCCCGGAAAATTCCATTTGGAAAGAACCTAAATCTCAGTGAGATGTTGATTGATGC |
| | TCCTACTATTAGTGAATGGAACCTCCAAGGTCTGCCAAATGATGACTTGTCCATTCAAAATGGAATT |
| | ATTGTCACGAAGGCATCTCGTTACCCTTTGTTAATTGATCCACAGACTCAAGGCAAGATCTGGATTA |
| | AAAATAAAGAAAGCCGAAATGAACTCCAGGTAACGTCTTTAAATCACAAGTACTTCAGAAACCACCT |
| | GGAAGACAGCCTTTCTCTTGGAAGGCCTTTGCTTATTGAAGATGTTGGAGAGGAACTAGATCCAGCA |
| | CTAGATAATGTTTTGGAAAGAAACTTCATTAAAACTGGGTCTACCTTTAAGGTGAAAGTTGGTGACA |
| | AGGAAGTAGATGTGTTGGATGGCTTTAGACTCTACATTACCACCAAATTGCCTAACCCAGCCTACAC |
| | CCCTGAGATAAGTGCCCGTACCTCCATCATTGACTTCACTGTCACCATGAAAGGTCTAGAAGATCAG |
| | TTACTGGGGAGGGTCATTCTCACAGAGAAGCAGGAATTGGAGAAAGAAAGAACTCATCTGATGGAAG |
| | ATGTAACTGCAAACAAAAGAAGGATGAAGGAACTAGAAGATAACTTGCTTTACCGCCTGACAAGTAC |
| | CCAGGGGTCCCTGGTAGAAGATGAAAGTCTCATTGTCGTGCTGAGTAACACAAAAAGGACAGCCGAG |
| | GAGGTGACACAGAAGCTAGAAATTTCTGCTGAGACAGAAGTTCAAATTAACTCAGCCCGGGAGGAAT |
| | ACAGACCAGTGGCTACGCGGGGCAGCATCCTCTACTTCCTCATTACTGAGATGCGCTTGGTTAATGA |
| | GATGTATCAGACTTCGCTTCGCCAGTTTCTGGGCTTATTTGACCTTTCCTTAGCCAGGTCTGTCAAG |
| | AGCCCGATTACAAGCAAGAGGATTGCTAATATCATCGAGCACATGACCTACGAGGTTTATAAGTATG |
| | CTGCCCGAGGGCTGTACGAGGAGCACAAATTCCTGTTCACCTTGTTGCTTACCCTAAAGATTGACAT |
| | CCAGAGGAACCGAGTCAAGCATGAAGAGTTTCTCACTCTTATTAAAGGAGGTGCCTCATTAGACCTT |
| | AAAGCTTGTCCTCCAAAACCATCAAAATGGATCCTGGACATAACATGGCTGAATTTGGTGGAACTTA |
| | GCAAACTCAGACAGTTTTCAGATGTCCTTGACCAGATATCGAGAAATGAGAAAATGTGGAAAATTTG |
| | GTTTGATAAGGAAAACCCGGAGGAGGAACCTCTTCCAAATGCCTATGATAAATCTCTTGACTGCTTC |
| | AGACGTCTTCTCCTTATTAGATCCTGGTGTCCTGACAGAACCATCTGGCAGGCCCGCAAGTACATCG |
| | TGGACTCCATGGGAGAAAAATATGCCGAAGGTGTTATTTTAGACTTGGAGAAGACGTGGGAGGAATC |
| | TGATCCACGGACGCCACTCATCTGTCTCCTGTCTATGGGCTCAGACCCCACAGATTCCATCATTGCC |
| | TTGGGGAAGAGATTAAAAATAGAAACCCGTTATGTGTCCATGGGCCAGGGCCAGGAAGTCCATGCTC |
| | GGAAGCTCTTGCAGCAGACCATGGCGAACGTAAGGCTGAATAGTCTACTTTTGTGCCATGCTGTTCA |
| | TATTACAGGTTATAGAATAGCAATGCAGAAGAAAAATATAAATCATTAGGAGTTTAAATTTACA |

| | | |
|---|---|---|
| ORF Start: ATG at 1 | | ORF Stop: TAG at 12241 |
| SEQ ID NO: 138 | 4080 aa | MW at 466144.8 kD |

| | |
|---|---|
| NOV17a, | MQRLKGEKEAKRALLDARHNYLFAIVASCLDLNKTEVEDAILEGNQIERIDQLFAVGGLRHLMFYYQ |
| CG161733-01 | |
| Protein Sequence | DVEEAETGQLGSLGGVNLVSGKIKKPKVFVTEGNDVALTGVCVFFIRTDPSKAITPDNIHQEVSFNM |
| | LDAADGGLLNSVRRLLSDIFIPALRATSHGWGELEGLQDAANIRQEFLSSLEGFVNVLSGAQESLKE |
| | KVNLRKCDILELKTLKEPTDYLTLANNPETLGKIEDCMKVWIKQTEQVLAENNQLLKEADDVGPRAE |
| | LEHWKKRLSKFNYLLEQLKSPDVKAVLAVLAAAKSKLLKTWREMDIRITDATNEAKDNVKYLYTLEK |
| | CCDPLYSSDPVSMMDAIPTLINAIKMIYSISHYYNTSEKITSLFVKVTNQIISACKAYITNNGTASI |
| | WNQPQDVVEEKILSAIFDFKEYQLCFHKTKQKLKQNPNAKQFDFSEMYIFGKFETFHRRLAKIIDIF |
| | TTLKTYSVLQDSTIEGLEDMATKYQVLYFKIKKKEYNFLDQRKMDFDQDYEEFCKQTNDLHVELRKF |
| | MDVTFAKIQNTNQALRMLKKFERAQILHFKLGIDDKYQLILENYGADIDMISKLYTKQKYDPPLARN |
| | QPPIAGKILWARQLFHRIQQPMQLFQQHPAVLSTAEAKPIIRSYNRMAKVLLEFEVLFHRAWLRQVA |
| | EIHVGLEASLLVKAPGTGELFVNFDPQILILFRETECMAQMGLEVSPLATSLFQFEGGAKALRLRTR |
| | KMLAEYQRVKSKIPAAIEQLIVPHLAKVDEALQPGLAALTWTSLNIEAYLENTFAKIKDLELLLDRV |
| | NDLIEFRIDAILEEMSSTPLCQLPQEEMTKVEEMVEPHADYSRNGAQILHFKSSLVEEAVNELVNML |
| | LDVEVLKISNENSVNYKNESSAKREEGNFDTLTSSINARANALLLTTVTRKKKETEMLGEEARELLS |
| | HFNHQNMDALLKVTRNTLEAIRKRIHSSHTINFRGNNLVPIFRASVTLAIPNIVMAPALEDVQQTLN |
| | KAVECIISVPKGVRQWSKIQERKMAALQSNEDSDSDVEMGENELQDTLEIASVNLPIPVQTKNYYKN |
| | VSENKEIVKLVSVLSTIINSTKKVCQEGLDCFKRYNHIWQKGKEEAIKTFITQSPLLSEFESQILYF |
| | QNLEQEINAEPEYVCVGSIALYTADLKFALTAETKAWMVVIGRHCNKKYRSEMENIFMLIEEFNKKL |
| | NRPIKDLDDIRIAMAALKEIREEQISIDFQVGPIEESYALLNRYGLLIAREEIDKVDTLHYAWEKLL |
| | ARAGEVQNKLVSLQPSFKKELISAVEVFLQDCHQFYLDYDLVCVQNGPMASGLKPQEASDRLIMFQV |
| | IFDNIYRKYITYTGGEELFGLPATQYPQLLEIKKQLNLLQKIYTLYNSVIETVNSYYDILWSEVNIE |
| | KINNELLEFQNRCRKLPRALKDWQAFLDLKKIIDDFSECCPLLEYMASKAMMERHWERITTLTGHSL |
| | DVGNESFKLRNIMEAPLLKYKEEIEDICISAVKERDIEQKLKQVINEWDNKTFTFGSFKTRGELLLR |
| | GDSTSEIIANMEDSLMLLGSLLSNRYNMPFKAQIQKWVQYLSNSTKIIESWMTVQNLWIYLEAVFVG |
| | GDIAKQLPKEAKRFSNIDKSWVKIMTRAHEVPSVVQCCVGDETLGQLLPHLLDQLEICQKSLTGYLE |
| | KKRLCFPRFFFVSDPALLEILGQASDSHTIQAHLLNVFDNIKSVKFHEKVIYDRILSISSQEGETIE |
| | LDKPVMAEGNVEVWLNSLLEESQSSLHLVIRQAAANIQETGFQLTEFLSSFPAQVGLLGIQMIWTRD |
| | SEEALRNAKFDKKIMQKTNQAFLELLNTLIDVTTRDLSSTERVKYETLITIHVHQRDIFDDLVHMHI |
| | KSPMDFEWLKQCRFYFNEDSDKNMIHITDVAFIYQNEFLGCTDRLVITPLTDRCYITLAQALGMSMG |
| | GAPAGPAGTGKTETTKDMGRCLGKYVVVFNCSDQMDFRGLGRIFKGLAQSGSWGCFDEFNRIDLPVL |
| | SVAAQQISIILTCKKEHKKSFIFTDGDNVTMNPEFGLFLTMNPGYAGRQELPENLKINFRSVAMMVP |
| | DRQIIIRVKLASCGFIDNVVLARKFFTLYKLCEEQLSKQVHYDFGLRNILSVLRTLGAAKRANPMDT |
| | ESTIVMRVLRDMNLSKLVDEDEPLFLSLIEDLFPNILLDKAGYPELEAAISRQVEEAGLINHPPWKL |
| | KVIQLFETQRVRHGMMTLGPSGAGKTTCIHTLMRAMTDCGKPHREMRMNPKAITAPQMFGRLDVATN |
| | DWTDGIFSTLWRKTLRAKKGEHIWIILDGPVDAIWIENLNSVLDDNKTLTLANGDRIPMAPNCKIIF |
| | EPHNIDNASPATVSRNGMVFMSSSILDWSPILQGFLKKRSPQEAEILRQLYTESFPDLYRFCIQNLE |
| | YKMEVLEAFVITQSINMLQGLIPLKEQGGEVSQAHLGRLFVFALLWSAGAALELDGRRRLELWLRSR |
| | PTGTLELPPPAGPGDTAFDYYVAPDGTWTHWNTRTQEYLYPSDTTPEYGSILVPNVDNVRTDFLIQT |
| | IAKQGKAVLLIGEQGTAKTVIIKGFMSKYDPECHMIKSLNFSSATTPLMFQRTIESYVDKRMGTTYG |
| | PPAGKKMTVFIDDVNMPIINEWGDQVTNEIVRQLMEQNGFYNLEKPGEFTSIVDIQFLAAMIHPGGG |
| | RNDIPQRLKRQFSIFNCTLPSEASVDKIFGVIGVGHYCTQRGFSEEVRDSVTKLVPLTRRLWQMTKI |
| | KMLPTPAKFHYVFNLRDLSRVWQGMLNTTSELLKLWKHECKRVIADRFTVSSDVTWFDKALVSLVEE |
| | EFGEEKKLLVDCGIDTYFVDFLRDAPEAAGGETSEEADAETPKIYEPIESFSHLKERLNMFLQLYNE |
| | SIRGAGMDMVFFADAMVHLVKISRVIRTPQGNALLVGVGGSGKQSLTRLASFIAGYVSFQITLTRSY |
| | NTSNLMEDLKVLYRTAGQQGKGITFIFTDNEIKDESFLEYMNNVLSSGEVSNLFARDEIDEINSDLA |
| | SVMKKEFPRCLPTNENLHDYFMSRVRQNLHIVLCFSPVGEKFRNRALKFPALISGCTIDWFSRWPKD |
| | ALVAVSEHFLTSYDIDCSLEIKKEVVQCMGSFQDGVAEKCVDYFQRFRRSTHVTPKSYLSFIQGYKF |
| | IYGEKHVEVRTLANRMNTGLEKLKEASESVAALSKELEAKEKELQVANDKADMVLKEVTMKAQAAEK |
| | VKAEVQKVKDRAQAIVDSISKDKAIAEEKLEAAKPALEEAEAALTIRPSDIATVRTLGRPPHLIMRI |
| | MDCVLLLFQRKVSAVKIDLEKSCTMPSWQESLKLMTAGNFLQNLQQFPKDTINEEVIEFLSPYFEMP |
| | DYNIETAKRVCGNVAGLCSWTKAMASFFSINKEVLPLKANLVVQENRHLLAMQDLQKAQAELDDKQA |
| | ELDVVQAEYEQAMTEKLLEDAERCRHKMQTASTLISGLAGEKERWTEQSQEFAAQTKRLVGDVLLAT |
| | AFLSYSGPFNQEFRDLLLNDWRKEMKARKIPFGKNLNLSEMLIDAPTISEWNLQGLPNDDLSIQNGI |
| | IVTKASRYPLLIDPQTQGKIWIKNKESRNELQVTSLNHKYFRNHLEDSLSLGRPLLIEDVGEELDPA |
| | LDNVLERNFIKTGSTFKVKVGDKEVDVLDGFRLYITTKLPNPAYTPEISARTSIIDFTVTMKGLEDQ |
| | LLGRVILTEKQELEKERTHLMEDVTANKRRMKELEDNLLYRLTSTQGSLVEDESLIVVLSNTKRTAE |
| | EVTQKLEISAETEVQINSAREEYRPVATRGSILYFLITEMRLVNEMYQTSLRQFLGLFDLSLARSVK |
| | SPITSKRIANIIEHMTYEVYKYAARGLYEEHKFLFTLLLTLKIDIQRNRVKHEEFLTLIKGGASLDL |
| | KACPPKPSKWILDITWLNLVELSKLRQFSDVLDQISRNEKMWKIWFDKENPEEEPLPNAYDKSLDCF |
| | RRLLLIRSWCPDRTIWQARKYIVDSMGEKYAEGVILDLEKTWEESDPRTPLICLLSMGSDPTDSIIA |
| | LGKRLKIETRYVSMGQGQEVHARKLLQQTMANVRLNSLLLCHAVHITGYRIAMQKKNINH |

Further analysis of the NOV17a protein yielded the following properties shown in Table 17B.

TABLE 17B
Protein Sequence Properties NOV17a

| SignalP analysis: | No Known Signal Sequence Predicted | | |
|---|---|---|---|
| PSORT II analysis: | PSG: | | a new signal peptide |
| | | | prediction method |
| | | | N-region: length 11; |
| | | | pos.chg 4; neg.chg 2 |
| | | | H-region: length 0; |
| | | | peak value −14.66 |
| | | | PSG score: −19.06 |
| | GvH: | | von Heijne's method for |
| | | | signal seq. recognition |
| | | | GyM score (threshold: −2.1): −9.75 |
| | | | possible cleavage site: |
| | | | between 24 and 25 |
| | >>>Seems to have no N-terminal signal peptide | | |
| | ALOM: | | Klein et al's method for TM |
| | | | region allocation |
| | | | Init position for calculation: 1 |
| | | | Tentative number of TMS(s) |
| | | | for the threshold 0.5: 3 |
| | | | Number of TMS(s) for |
| | | | threshold 0-5: 0 |
| | | | PERIPHERAL Likelihood = 1.06 |
| | | | (at 2081) |
| | | | ALOM score: −1.12 |
| | | | (number of TMSs: 0) |
| | MITDISC: | | discrimination of mitochondrial |
| | | | targeting seq |
| | | | R content: 1 Hyd Moment(75): 11.39 |
| | | | Hyd Moment(95): 5.73 G content: 1 |
| | | | D/E content: 2 S/T content: 0 |
| | | | Score: −5.92 |
| | Gavel: | | prediction of cleavage sites |
| | | | for mitochondrial preseq |
| | | | R-2 motif at 13 QRL\|KG |
| | NUCDISC: | | discrimination of nuclear |
| | | | localization signals |
| | | | pat4: KKPK (4) at 91 |
| | | | pat4: RKKK (5) at 921 |
| | | | pat7: PQRLKRQ (4) at 2685 |
| | | | bipartite: none |
| | | | content of basic residues: 12.2% |
| | | | NLS Score: 0.44 |
| | KDEL: | | ER retention motif in |
| | | | the C-terminus: none |
| | ER Membrane Retention Signals: | | |
| | XXRR-like motif in the N-terminus: QRLK | | |
| | KKXX-like motif in the C-terminus: KNIN | | |
| | SKL: | | peroxisomal targeting signal in |
| | | | the C-terminus: none |
| | PTS2: | | 2nd peroxisomal targeting |
| | | | signal: found |
| | | | KIPAAIEQL at 749 |
| | VAC: | | possible vacuolar |
| | | | targeting motif: found |
| | | | KLPN at 3656 |
| | RNA-binding motif: none | | |
| | Actinin-type actin-binding motif: | | |
| | | | type 1: none |
| | | | type 2: none |
| | NNYR: | | N-myristoylation pattern : none |
| | Prenylation motif: none | | |
| | memYQRL: | | transport motif from |
| | | | cell surface to Golgi: none |
| | Tyrosines in the tail: none | | |
| | Dileucine motif in the tail: none | | |
| | checking 63 PROSITE DNA binding motifs: none | | |
| | checking 71 PROSITE ribosomal | | |
| | protein motifs: none | | |
| | checking 33 PROSITE prokaryotic | | |
| | DNA binding motifs: | | |
| | Bacterial regulatory proteins, lysR family signature | | |
| | (PS00044) : *** found *** | | |
| | IFRASVTLAIPNIVMAPALEDVQQTL at 979 | | |
| | NNCN: | | Reinhardt's method for Cytoplasmic/ |
| | | | Nuclear discrimination |
| | | | Prediction: cytoplasmic |
| | | | Reliability: 89 |
| | COIL: Lupas's algorithm to | | |
| | detect coiled-coil regions | | |
| | 8780 | L | 0.63 |
| | 781 | N | 0.96 |
| | 782 | I | 0.96 |
| | 783 | E | 0.96 |
| | 784 | A | 0.96 |
| | 785 | Y | 0.96 |
| | 786 | L | 0.96 |
| | 787 | E | 0.96 |
| | 788 | N | 0.96 |
| | 789 | T | 0.96 |
| | 790 | F | 0.96 |
| | 791 | A | 0.96 |
| | 792 | K | 0.96 |
| | 793 | I | 0.96 |
| | 794 | K | 0.96 |
| | 795 | D | 0.96 |
| | 796 | L | 0.96 |
| | 797 | E | 0.96 |
| | 798 | L | 0.96 |
| | 799 | L | 0.96 |
| | 800 | L | 0.96 |
| | 801 | D | 0.96 |
| | 802 | R | 0.96 |
| | 803 | V | 0.96 |
| | 804 | N | 0.96 |
| | 805 | D | 0.96 |
| | 806 | L | 0.96 |
| | 807 | I | 0.96 |
| | 808 | E | 0.96 |
| | 809 | F | 0.84 |
| | 1022 | K | 0.57 |
| | 1023 | I | 0.62 |
| | 1024 | Q | 0.62 |
| | 1025 | E | 0.62 |
| | 1026 | R | 0.62 |
| | 1027 | K | 0.62 |
| | 1028 | M | 0.62 |
| | 1029 | A | 0.62 |
| | 1030 | A | 0.62 |
| | 1031 | L | 0.62 |
| | 1032 | Q | 0.62 |
| | 1033 | S | 0.62 |
| | 1034 | N | 0.62 |
| | 1035 | E | 0.62 |
| | 1036 | D | 0.62 |
| | 1037 | S | 0.62 |
| | 1038 | D | 0.62 |
| | 1039 | S | 0.62 |
| | 1040 | D | 0.62 |
| | 1041 | V | 0.62 |
| | 1042 | E | 0.62 |
| | 1043 | M | 0.62 |
| | 1044 | G | 0.62 |
| | 1045 | E | 0.62 |
| | 1046 | N | 0.62 |
| | 1047 | E | 0.62 |
| | 1048 | L | 0.62 |
| | 1049 | Q | 0.62 |
| | 1050 | D | 0.62 |
| | 1051 | T | 0.55 |
| | 1197 | L | 0.52 |
| | 1198 | I | 0.52 |
| | 1199 | E | 0.52 |
| | 1200 | E | 0.52 |
| | 1201 | F | 0.52 |
| | 1202 | N | 0.52 |
| | 1203 | K | 0.52 |
| | 1204 | K | 0.52 |
| | 1205 | L | 0.52 |
| | 1206 | N | 0.52 |
| | 1207 | R | 0.52 |
| | 1208 | P | 0.52 |
| | 1209 | I | 0.52 |
| | 1210 | K | 0.52 |
| | 1211 | D | 0.52 |
| | 1212 | L | 0.52 |
| | 1213 | D | 0.52 |
| | 1214 | D | 0.52 |
| | 1215 | I | 0.52 |
| | 1216 | R | 0.52 |
| | 1217 | I | 0.52 |
| | 1218 | A | 0.52 |
| | 1219 | M | 0.52 |
| | 1220 | A | 0.52 |
| | 1221 | A | 0.52 |
| | 1222 | L | 0.52 |
| | 1223 | K | 0.52 |
| | 1224 | H | 0.52 |
| | 3154 | H | 0.56 |
| | 3155 | V | 0.69 |
| | 3156 | E | 0.93 |
| | 3157 | V | 0.93 |
| | 3158 | R | 0.97 |
| | 3159 | T | 0.99 |
| | 3160 | L | 0.99 |
| | 3161 | A | 0.99 |
| | 3162 | N | 0.99 |
| | 3163 | R | 0.99 |
| | 3164 | M | 0.99 |
| | 3165 | N | 0.99 |
| | 3166 | T | 1.00 |
| | 3167 | G | 1.00 |
| | 3168 | L | 1.00 |
| | 3169 | E | 1.00 |
| | 3170 | K | 1.00 |
| | 3171 | L | 1.00 |
| | 3172 | K | 1.00 |
| | 3173 | E | 1.00 |
| | 3174 | A | 1.00 |
| | 3175 | S | 1.00 |
| | 3176 | E | 1.00 |
| | 3177 | S | 1.00 |
| | 3178 | V | 1.00 |
| | 3179 | A | 1.00 |
| | 3180 | A | 1.00 |
| | 3181 | L | 1.00 |
| | 3182 | S | 1.00 |
| | 3183 | K | 1.00 |
| | 3184 | E | 1.00 |
| | 3185 | L | 1.00 |
| | 3186 | E | 1.00 |
| | 3187 | A | 1.00 |
| | 3188 | K | 1.00 |
| | 3189 | E | 1.00 |
| | 3190 | K | 1.00 |
| | 3191 | E | 1.00 |
| | 3192 | L | 1.00 |
| | 3193 | Q | 1.00 |
| | 3194 | V | 1.00 |
| | 3195 | A | 1.00 |
| | 3196 | N | 1.00 |
| | 3197 | D | 1.00 |
| | 3198 | K | 1.00 |
| | 3199 | A | 1.00 |
| | 3200 | D | 1.00 |
| | 3201 | M | 1.00 |
| | 3202 | V | 1.00 |
| | 3203 | L | 1.00 |
| | 3204 | K | 1.00 |
| | 3205 | E | 1.00 |
| | 3206 | V | 1.00 |
| | 3207 | T | 0.99 |
| | 3208 | M | 0.99 |
| | 3209 | K | 0.99 |
| | 3210 | A | 0.99 |
| | 3211 | Q | 0.99 |
| | 3212 | A | 0.98 |
| | 3213 | A | 0.98 |
| | 3214 | E | 0.98 |
| | 3215 | K | 0.98 |
| | 3216 | V | 0.98 |
| | 3217 | K | 0.98 |
| | 3218 | A | 0.98 |
| | 3219 | E | 0.99 |
| | 3220 | V | 0.99 |
| | 3221 | Q | 0.99 |
| | 3222 | K | 0.99 |
| | 3223 | V | 0.99 |
| | 3224 | K | 0.99 |
| | 3225 | D | 0.99 |
| | 3226 | R | 0.99 |
| | 3227 | A | 0.99 |
| | 3228 | Q | 0.99 |
| | 3229 | A | 0.99 |
| | 3230 | I | 0.99 |
| | 3231 | V | 0.99 |
| | 3232 | D | 0.99 |
| | 3233 | S | 0.99 |
| | 3234 | I | 0.99 |
| | 3235 | S | 0.99 |
| | 3236 | K | 0.99 |
| | 3237 | D | 0.99 |
| | 3238 | K | 0.99 |
| | 3239 | A | 0.99 |
| | 3240 | I | 0.99 |
| | 3241 | A | 0.99 |
| | 3242 | E | 0.99 |
| | 3243 | E | 0.99 |
| | 3244 | K | 0.99 |
| | 3245 | L | 0.99 |
| | 3246 | E | 0.99 |
| | 3247 | A | 0.99 |
| | 3248 | A | 0.99 |
| | 3249 | K | 0.98 |
| | 3386 | L | 0.96 |
| | 3387 | K | 0.96 |
| | 3388 | A | 0.96 |
| | 3389 | N | 0.96 |
| | 3390 | L | 0.96 |
| | 3391 | V | 0.96 |
| | 3392 | V | 0.96 |
| | 3393 | Q | 0.96 |
| | 3394 | E | 0.96 |
| | 3395 | N | 0.96 |
| | 3396 | R | 0.96 |
| | 3397 | H | 0.96 |
| | 3398 | L | 0.97 |
| | 3399 | L | 1.00 |
| | 3400 | A | 1.00 |
| | 3401 | M | 1.00 |
| | 3402 | Q | 1.00 |
| | 3403 | D | 1.00 |
| | 3404 | L | 1.00 |
| | 3405 | Q | 1.00 |
| | 3406 | K | 1.00 |
| | 3407 | A | 1.00 |
| | 3408 | Q | 1.00 |
| | 3409 | A | 1.00 |
| | 3410 | E | 1.00 |
| | 3411 | L | 1.00 |
| | 3412 | D | 1.00 |
| | 3413 | D | 1.00 |
| | 3414 | K | 1.00 |
| | 3415 | Q | 1.00 |
| | 3416 | A | 1.00 |
| | 3417 | E | 1.00 |
| | 3418 | L | 1.00 |
| | 3419 | D | 1.00 |
| | 3420 | V | 1.00 |
| | 3421 | V | 1.00 |
| | 3422 | Q | 1.00 |
| | 3423 | A | 1.00 |
| | 3424 | E | 1.00 |
| | 3425 | Y | 1.00 |
| | 3426 | E | 1.00 |
| | 3427 | Q | 1.00 |
| | 3428 | A | 1.00 |
| | 3429 | M | 1.00 |
| | 3430 | T | 1.00 |
| | 3431 | E | 1.00 |
| | 3432 | K | 1.00 |
| | 3433 | L | 0.99 |
| | 3434 | L | 0.96 |
| | 3435 | E | 0.90 |
| | 3436 | D | 0.69 |
| | 3437 | A | 0.69 |
| | 3438 | E | 0.69 |
| | 3439 | R | 0.52 |
| | 3690 | I | 0.82 |
| | 3691 | L | 0.96 |
| | 3692 | T | 1.00 |
| | 3693 | E | 1.00 |
| | 3694 | K | 1.00 |
| | 3695 | Q | 1.00 |
| | 3696 | E | 1.00 |
| | 3697 | L | 1.00 |
| | 3698 | E | 1.00 |
| | 3699 | K | 1.00 |
| | 3700 | E | 1.00 |
| | 3701 | R | 1.00 |
| | 3702 | T | 1.00 |
| | 3703 | H | 1.00 |
| | 3704 | L | 1.00 |
| | 3705 | M | 1.00 |
| | 3706 | E | 1.00 |
| | 3707 | D | 1.00 |
| | 3708 | V | 1.00 |
| | 3709 | T | 1.00 |
| | 3710 | A | 1.00 |
| | 3711 | N | 1.00 |
| | 3712 | K | 1.00 |
| | 3713 | R | 1.00 |
| | 3714 | R | 1.00 |
| | 3715 | M | 1.00 |
| | 3716 | K | 1.00 |
| | 3717 | E | 1.00 |
| | 3718 | L | 1.00 |
| | 3719 | E | 1.00 |
| | 3720 | D | 1.00 |
| | 3721 | N | 1.00 |
| | 3722 | L | 1.00 |
| | 3723 | L | 0.99 |
| | 3724 | Y | 0.78 |

total: 273 residues

Final Results (k = 9/23)

```
47.8 %: nuclear
34.8 %: cytoplasmic
  8.7 %: mitochondrial
  4.3 %: vacuolar
  4.3 %: vesicles of secretory system
```

>>prediction for CG161733-01 is nuc (k = 23)

A search of the NOV17a protein against the Geneseq database, a proprietary database that contains sequences published in patents and patent publication, yielded several homologous proteins shown in Table 17C.

TABLE 17C
Geneseq Results for NOV17a

| | | NOV17a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABB64461 | Drosophila melanogaster | 2 . . . 3472 | 1789/3581 (49%) | 0.0 |
| | polypeptide SEQ ID NO | 9 . . . 3500 | 2381/3581 (65%) | |
| | 20175 - Drosophila | | | |
| | melanogaster, 3508 aa. | | | |
| | [WO200171042-A2, | | | |
| | 27-SEP-2001] | | | |
| ABB60101 | Drosophila melanogaster | 272 . . . 4042 | 1159/3884(29%) | 0.0 |
| | polypeptide SEQ ID NO | 256 . . . 3927 | 1923/3884 (48%) | |
| | 7095 - Drosophila | | | |
| | melanogaster, 4472 aa. | | | |
| | [WO200171042-A2, | | | |
| | 27-SEP-2001] | | | |
| ABB58592 | Drosophila melanogaster | 735 . . . 4046 | 1040/3428 (30%) | 0.0 |
| | polypeptide SEQ ID NO | 1210 . . . 4460 | 1741/3428 (50%) | |
| | 2568 - Drosophila | | | |
| | melanogaster, 4820 aa. | | | |
| | [WO200171042-A2, | | | |
| | 27-SEP-2001] | | | |
| ABB61520 | Drosophila melanogaster | 1279 . . . 4062 | 949/2885 (32%) | 0.0 |
| | polypeptide SEQ ID NO | 642 . . . 3464 | 1517/2885 (51%) | |
| | 11352 - Drosophila | | | |
| | melanogaster, 4010 aa. | | | |
| | [WO200171042-A2, | | | |
| | 27-SEP-2001] | | | |
| ABB62958 | Drosophila melanogaster | 1151 . . . 4062 | 940/2994 (31%) | 0.0 |
| | polypeptide SEQ ID NO | 609 . . . 3520 | 1530/2994 (50%) | |
| | 15666 - Drosophila | | | |
| | melanogaster, 4081 aa. | | | |
| | [WO200171042-A2, | | | |
| | 27-SEP-2001] | | | |

In a BLAST search of public sequence datbases, the NOV17a protein was found to have homology to the proteins shown in the BLASTP data in Table 17D.

TABLE 17D
Public BLASTP Results for NOV17a

| | | NOV17a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q8TE73 | Axonemal dynein heavy | 2 . . . 4062 | 3986/4092 (97%) | 0.0 |
| | chain DNAH5 - Homo | 20 . . . 4103 | 4000/4092 (97%) | |
| | sapiens (Human), 4624 aa. | | | |
| Q8VHE6 | Axonemal dynein heavy | 2 . . . 4062 | 3554/4090 (86%) | 0.0 |
| | chain 5 - Mus musculus | 20 . . . 4100 | 3800/4090 (92%) | |
| | (Mouse), 4621 aa. | | | |
| Q91XP9 | Axonemal dynem heavy | 3 . . . 4053 | 2472/4072 (60%) | 0.0 |
| | chain 8 short form - Mus | 158 . . . 4200 | 3080/4072 (74%) | |
| | musculus (Mouse), 4202 aa. | | | |
| Q91XQ0 | Axonemal dynein heavy | 3 . . . 4062 | 2474/4081(60%) | 0.0 |
| | chain 8 long form - Mus | 158 . . . 4209 | 3082/4081 (74%) | |
| | musculus (Mouse), 4731 aa. | | | |
| Q91XP8 | Axonemal dynein heavy | 3 . . . 4062 | 2471/4081 (60%) | 0.0 |
| | chain 8 long form - Mus | 158 . . . 4209 | 3079/4081 (74%) | |
| | musculus (Mouse), 4731 aa. | | | |

PFam analysis predicts that the NOV17a protein contains the domains shown in the Table 17E.

TABLE 17E
Domain Analysis of NOV17a

| | | Identities/ | |
|---|---|---|---|
| | NOV17a | Similarities for | |
| Pfam Domain | Match Region | the Matched Region | Expect Value |
| AAA | 2225 . . . 2416 | 31/247 (13%) | 0.65 |
| | | 113/247 (46%) | |

### Example 18

The NOV18 clone was analyzed, and the nucleotide and encoded polypeptide sequences are shown in Table 18A.

TABLE 18A
NOV18 Sequence Analysis

| | |
|---|---|
| SEQ ID NO: 139 | 770 bp |

| | |
|---|---|
| NOV18a, | GAATCGCCCTTAGATATGTGTAACACACCAACTTACTGTGACCTGGGAAAGGCTGCTAAGGATGTCT |
| CG161762-01 | |
| DNA Sequence | TCAACAAAGGATATGGCTTTGGCATGGGGAAGATAGACCTGAAAACCAAGTCCTGTAGTGGAGTGAT |
| | GGAATTTTCTACTTCTGGTCATGCTTACACTGATACAGGGAAAGCATCAGGCAACCTAGAAACCAAA |
| | TATAAGGTCTGTAACTATGGACTTACCTTCACCCAGAAATGGAACACAGACAATACTCTAGGGACAG |
| | AAATCTCTTGGGAGAATAAGTTGGCTGAAGGGTTGAAACTGACTCTTGATACCATATTTGTACCGAA |
| | CACAGGAAAGAAGAGTGGGAAATTGAAGGCCTCCTATAAACGGGATTGTTTTAGTGTTGGCAGTAAT |
| | GTTGATATAGATTTTTCTGGACCAACCATCTATGGCTGGGCTGTGTTGGCCTTCGAAGGGTGGCTTG |
| | CTGGCTATCAGATGAGTTTTGACACAGCCAAATCCAAACTGTCACAGAATAATTTCGCCCTGGGTTA |
| | CAAGGCTGCGGACTTCCAGCTGCACACACATGTGAACGATGGCACTGAATTTGGAGGTTCTATCTAC |
| | CAGAAGGCTAAAGTAAATAATGCCAGCCTGATTGGACTGGGTTATACTCAGACCCTTCGACCAGGAG |
| | TCAAATTGACTTTATCAGCTTTAATCGATGGGAAGAACTTCAGTGCAGGAGGTCACAAGGTTGGCTT |
| | GGGATTTGAACTGCAAGCTTAATGTGGTTTGAG |

| | | |
|---|---|---|
| ORF Start: ATG at 16 | | ORF Stop: TAA at 757 |
| SEQ ID NO: 140 | 247 aa | MW at 26688.0 kD |

| | |
|---|---|
| NOV18a, | MCNTPTYCDLGKAAKDVFNKGYGFGMGKIDLKTKSCSGVMEFSTSGHAYTDTGKASGNLETKYKVCN |
| CG161762-01 | |
| Protein Sequence | YGLTFTQKWNTDNTLGTEISWENKLAEGLKLTLDTIFVPNTGKKSGKLKASYKRDCFSVGSNVDIDF |
| | SGPTIYGWAVLAFEGWLAGYQMSFDTAKSKLSQNNFALGYKAADFQLHTHVNDGTEFGGSIYQKAKV |
| | NNASLIGLGYTQTLRPGVKLTLSALIDGKNFSAGGHKVGLGFELQA |

Further analysis of the NOV18a protein yielded the following properties shown in Table 18B.

TABLE 18B
Protein Sequence Properties NOV18a

| SignalP analysis: | No Known Signal Sequence Predicted | |
|---|---|---|
| PSORT II analysis: | PSG: | a new signal peptide |
| | | prediction method |
| | | N-region: length 9; pos.chg 0; |
| | | neg.chg 1 |
| | | H-region: length 2; peak value 0.00 |
| | | PSG score: −4.40 |
| | GvH: | von Heijne's method for signal |
| | | seq. recognition |
| | | GvH score (threshold: −2.1): −9.23 |
| | | possible cleavage site: |
| | | between 50 and 51 |
| | >>>Seems to have no N-terminal signal peptide | |
| | ALOM: | Klein et al's method for |
| | | TM region allocation |
| | | Init position for calculation: 1 |
| | | Tentative number of TMS(s) |
| | | for the threshold 0.5: 0 |
| | | number of TMS(s) . . . fixed |
| | | PERIPHERAL |
| | | Likelihood = 6.47 (at 138) |
| | | ALOM score: 6.47 (number |
| | | of TMSs: 0) |
| | MITDISC: | discrimination of mitochondrial |
| | | targeting seq |
| | | content: 0 Hyd Moment(75): 3.36 |
| | | Hyd Moment(95): 4.93 G content: 1 |
| | | D/E content: 2 S/T content: 2 |
| | | Score: −7.44 |
| | Gavel: | prediction of cleavage sites |
| | | for mitochondrial preseq |
| | | cleavage site motif not found |
| | NUCDISC: | discrimination of nuclear |
| | | localization signals |
| | | pat4: none |
| | | pat7: none |
| | | bipartite: none |
| | | content of basic residues: 10.9% |
| | | NLS Score: −0.47 |
| | KDEL: | ER retention motif in |
| | | the C-terminus: none |
| | ER Membrane Retention Signals: none | |
| | SKL: | peroxisomal targeting signal |
| | | in the C-terminus: none |
| | PTS2: | 2nd peroxisonal targeting |
| | | signal: none |
| | VAC: | possible vacuolar |
| | | targeting motif: none |
| | RNA-binding motif: none | |
| | Actinin-type actin-binding motif: | |
| | | type 1: none |
| | | type 2: none |
| | NMYR: | N-myristoylation pattern : none |
| | Prenylation motif: none | |
| | memYQRL: | transport motif from |
| | | cell surface to Golgi: none |
| | | Tyrosines in the tail: none |
| | | Dileucine motif in the tail: none |
| | | checking 63 PROSITE DNA |
| | | binding motifs: none |
| | | checking 71 PROSITE ribosomal |
| | | protein motifs: none |
| | | checking 33 PROSITE prokaryotic |
| | | DNA binding motifs: none |
| | NNCN: | Reinhardt's method for Cytoplasmic/ |
| | | Nuclear discrimination |
| | | Prediction: cytoplasmic |
| | | Reliability: 89 |
| | COIL: | Lupas's algorithm to detect |
| | | coiled-coil regions |
| | | total: 0 residues |

Final Results (k = 9/23):

```
65.2 %: cytoplasmic
26.1 %: nuclear
  4.3 %: Golgi
  4.3 %: mitochondrial
```

>>prediction for CG161762-0l is cyt (k = 23)

A search of the NOV18a protein against the Geneseq database, a proprietary database that contains sequences published in patents and patent publication, yielded several homologous proteins shown in Table 18C.

TABLE 18C
Geneseq Results for NOV18a

| | | NOV18a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP41522 | Human ovarian antigen | 1 . . . 247 | 244/284 (85%) | e−137 |
| | HVVAQ70, SEQ ID | 2 . . . 284 | 245/284 (85%) | |
| | NO:2654 - Homo sapiens, | | | |
| | 284 aa. [WO200200677-A1, | | | |
| | 03-JAN-2002] | | | |
| AAY45015 | Protein encoded by fchd545 | l . . . 247 | 244/284 (85%) | e−137 |
| | gene - Homo sapiens, 283 aa. | 1 . . . 283 | 245/284 (85%) | |
| | [WO200006206-A1, | | | |
| | 10-FEB-2000] | | | |
| AAY07222 | Voltage-dependent anion | 1 . . . 247 | 244/284 (85%) | e−137 |
| | channel CBMAAD07 protein | 1 . . . 283 | 245/284 (85%) | |
| | sequence - Homo sapiens, | | | |
| | 283 aa. [WO9921990-A1, | | | |
| | 06-MAY-1999] | | | |
| AAW48908 | Human high | l . . . 247 | 244/284 (85%) | e−137 |
| | voltage-dependent anion | 1 . . . 283 | 245/284 (85%) | |
| | channel protein - Homo | | | |
| | sapiens, 283 aa. | | | |
| | [US5780235-A, 14-JUL-1998] | | | |
| AAW36004 | Human Fchd545 gene | 1 . . . 247 | 244/284 (85%) | e−137 |
| | product - Homo sapiens, 283 aa. | 1 . . . 283 | 245/284 (85%) | |
| | [WO9730065-A1, 21-AUG-1997] | | | |

In a BLAST search of public sequence datbases, the NOV18a protein was found to have homology to the proteins shown in the BLASTP data in Table 18D.

TABLE 18D
Public BLASTP Results for NOV18a

| | | | Identities/ | |
|---|---|---|---|---|
| Protein | | | Similarities for | |
| Accession | | NOV18a Residues/ | the Matched | Expect |
| Number | Protein/Organism/Length | Match Residues | Portion | Value |
| Q96J36 | Voltage-dependent anion | 1 . . . 247 | 245/284 (86%) | e−138 |
| | channel 3 - Homo sapiens | 1 . . . 284 | 246/284 (86%) | |
| | (Human), 284 aa. | | | |
| Q9Y277 | Voltage-dependent | 1 . . . 247 | 244/284 (85%) | e−136 |
| | anion-selective channel | 1 . . . 283 | 245/284 (85%) | |
| | protein 3 (VDAC-3) | | | |
| | (hVDAC3) (Outer | | | |
| | mitochondrial membrane | | | |
| | protein porin 3) - Homo | | | |
| | sapiens (Human), 283 aa. | | | |
| Q60931 | Voltage-dependent | 1 . . . 247 | 241/284 (84%) | e−135 |
| | anion-selective channel | 1 . . . 283 | 245/284 (85%) | |
| | protein 3 (VDAC-3) | | | |
| | (mVDAC3) (Outer | | | |
| | mitochondrial membrane | | | |
| | protein porin 3) - Mus | | | |
| | musculus (Mouse), 283 aa. | | | |
| Q9MZ13 | Voltage-dependent | 1 . . . 247 | 240/284 (84%) | e−135 |
| | anion-selective channel | 1 . . . 283 | 245/284 (85%) | |
| | protein 3 (VDAC-3) | | | |
| | (hVDAC3) (Outer | | | |
| | mitochondrial membrane | | | |
| | protein porin 3) - Bos taurus | | | |
| | (Bovine), 283 aa. | | | |
| Q9TT13 | Voltage-dependent | 1 . . . 247 | 240/284 (84%) | e−134 |
| | anion-selective channel | 1 . . . 283 | 244/284 (85%) | |
| | protein 3 (VDAC-3) (Outer | | | |
| | mitochondrial membrane | | | |
| | protein porin 3) - Oryctolagus | | | |
| | cuniculus (Rabbit), 283 aa. | | | |

PFam analysis predicts that the NOV18a protein contains the domains shown in the Table 18E.

TABLE 18E
Domain Analysis of NOV18a

| | | Identities/ | |
|---|---|---|---|
| | | Similarities | |
| | NOV18a | for the | |
| Pfam | Match | Matched | Expect |
| Domain | Region | Region | Value |
| Euk_porin | 2 . . . 247 | 143/290 (49%) | 1.1e−133 |
| | | 242/290 (83%) | |

### Example 19

The NOV19 clone was analyzed, and the nucleotide and encoded polypeptide sequences are shown in Table 19A.

TABLE 19A
NOV19 Sequence Analysis

| | |
|---|---|
| SEQ ID NO: 141 | 1914 bp |

| | |
|---|---|
| NOV19a, | AAGGAACCAACATAAAGAGAAATGCAGACGATATAACCAGTAATGACCATGGTGAAGATAAAGATAT |
| CG162855-01 | |
| DNA Sequence | TCATGAACAGAACAGTAAGAAGCCTGTTATGGTCTATATCCATGGGGGATCTTACATGGAGGGAACC |
| | GGTAACATGATTGATGGCAGCATTTTGGCCAGCTATGGGAACGTCATCGTTATCACCATTAACTACC |
| | GTCTGGGAATACTAGGTCTCTTCCAGAAGGCCATCATTCAGAGCGGCACTGCCCTGTCCAGCTGGGC |
| | AGTGAACTACCAGCCGGCCAAGTACACTCGGATATTGGCAGACAAGGTCGGCTGCAACATGCTGGAC |
| | ACCACGGACATGGTAGAATGTCTGAAGAACAAGAACTACAAGGAGCTCATCCAGCAGACCATCACCC |
| | CGGCCACCTACCACATAGCCTTTGGGCCGGTGATCGACGGCGACGTCATCCCAGACGGCCCCCAGAT |
| | CCTGATGGAGCAAGGCGAGTTCCTCAACTACGACATCATGCTGGGCGTCAACCAAGGGGAAGGCCTG |
| | AAGTTCGTGGACGGCATCGTGGATAACGAGGACGGTGTGACGCCCAACGACTTTGACTTCTCCGTGT |
| | CCAACTTCGTGGACAACCTTTACGGCTACCCTGAAGGGAAAGACACTTTGCGGGAGACTATCAAGTT |
| | CATGTACACAGACTGGGCCGATAAGGAAAACCCGGAGACGCGGCGGAAAACCCTGGTGGCTCTCTTT |
| | ACTGACCATCAGTGGGTGGCCCCCGCCGTGGCCACCGCCGACCTGCACGCGCAGTACGGCTCCCCCA |
| | CCTACTTCTATGCCTTCTATCATCACTGCCAAAGCGAAATGAAGCCCAGCTGGGCAGATTCGGCCCA |
| | TGGCGATGAAGTCCCCTATGTCTTCGGCATCCCCATGATCGGTCCCACAGAGCTCTTCAGTTGTAAT |
| | TTCTCCAAGAACGACGTCATGCTCAGTGCCGTGGTGATGACCTACTGGACGAACTTCGCCAAAACTG |
| | GTGATCCAAACCAACCAGTTCCTCAGGATACCAAGTTCATTCATACAAAACCCAATCGCTTTGAAGA |
| | AGTGGCCTGGTCCAAGTATAATCCCAAAGACCAGCTCTATCTGCATATTGGCTTGAAACCCAGAGTG |
| | AGAGATCACTACCGGGCAACGAAAGTGGCTTTCTGGTTGGAATTGGTTCCTCATTTGCACAACTTGA |
| | ACGAGATATTCCAGTATGTTTCAACAACCACAAAGGTTCCTCCACCAGACATGACATCATTTCCCTA |
| | TGGCACCCGGCGATCTCCCGCCAAGATATGGCCAACCACCAAACGCCCAGCAATCACTCCTGCCAAC |
| | AATCCCAAACACTCTAAGGACCCTCACAAAACAGGGCCCGAGGACACAACTGTCCTCATTGAAACCA |
| | AACGAGATTATTCCACCGAATTAAGTGTCACCATTGCCGTCGGGGCGTCGCTCCTCTTCCTCAACAT |
| | CTTAGCCTTTGCGGCGCTGTACTACAAAAAGGACAAGAGGCGCCATGAGACTCACAGGCACCCCAGT |
| | CCCCAGAGAAACACCACAAATGATATCACTCACATCCAGAACGAAGAGATCATGTCTCTGCAGATGA |
| | AGCAGCTGGAACACGATCACGAGTGTGAGTCGTTGCAGGCACACGACACGCTGAGGCTCACCTGCCC |
| | TCCAGACTACACCCTCACGCTGCGCCGGTCGCCGGATGACATCCCATTTATGACGCCAAACACCATC |
| | ACCATGATTCCAAACACATTGATGGGGATGCAGCCTTTACACACTTTTAAAACCTTCAGTGGAGGAC |
| | AAAACAGTACAAATTTACCCCACGGACATTCCACCACTAGAGTATAGCTTTTCCCTATTTCCCCTCC |
| | TATCCCTCTGCCCCTACTGCTCAGCAATGTAAAAGAGA |

| | | |
|---|---|---|
| ORF Start: ATG at 96 | | ORF Stop: TAG at 1854 |
| SEQ ID NO: 142 | 586 aa | MW at 66369.7 kD |

| | |
|---|---|
| NOV19a, | MVYIHGGSYMEGTGNMIDGSILASYGNVIVITINYRLGILGLFQKAIIQSGTALSSWAVNYQPAKYT |
| CG162855-01 | |
| Protein Sequence | RILADKVGCNMLDTTDMVECLKNKNYKELIQQTITPATYHIAFGPVIDGDVIPDGPQILMEQGEFLN |
| | YDIMLGVNQGEGLKFVDGIVDNEDGVTPNDFDFSVSNFVDNLYGYPEGKDTLRETIKFMYTDWADKE |
| | NPETRRKTLVALFTDHQWVAPAVATADLHAQYGSPTYFYAFYHHCQSEMKPSWADSAHGDEVPYVFG |
| | IPMIGPTELFSCNFSKNDVMLSAVVMTYWTNFAKTGDPNQPVPQDTKFIHTKPNRFEEVAWSKYNPK |
| | DQLYLHIGLKPRVRDHYRATKVAFWLELVPHLHNLNEIFQYVSTTTKVPPPDMTSFPYGTRRSPAKI |
| | WPTTKRPAITPANNPKHSKDPHKTGPEDTTVLIETKRDYSTELSVTIAVGASLLFLNILAFAALYYK |
| | KDKRRHETHRHPSPQRNTTNDITHIQNEEIMSLQMKQLEHDHECESLQAHDTLRLTCPPDYTLTLRR |
| | SPDDIPFMTPNTITMIPNTLMGMQPLHTFKTFSGGQNSTNLPHGHSTTRV |

Further analysis of the NOV19a protein yielded the following properties shown in Table 19B.

TABLE 19B
Protein Sequence Properties NOV19a

| SignalP | | | | | |
|---|---|---|---|---|---|
| analysis: | No Known Signal Sequence Predicted | | | | |
| PSORT II | PSG: a new signal peptide prediction method | | | | |
| analysis: | N-region: length 11; pos.chg 0; neg.chg 1 | | | | |
| | H-region: length 6; peak value 0.00 | | | | |
| | PSG score: −4.40 | | | | |
| | GvH: von Heijne's method for signal seq. recognition | | | | |
| | GvH score (threshold: −2.1): −4.97 | | | | |
| | possible cleavage site: between 58 and 59 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 2 | | | | |
| | Number of TMS(s) for threshold 0.5: 1 | | | | |
| | INTEGRAL | Likelihood = | −6.05 | Transmembrane | 449-465 |

| | | |
|---|---|---|
| PERIPHERAL | Likelihood = | 5.09 (at 266) |

| ALOM score: −6.05 (number of TMSs: 1) | | | |
|---|---|---|---|
| MTOP: Prediction of membrane topology (Hartmann et al.) | | | |
| Center position for calculation: 456 | | | |
| Charge difference: 7.5 C( 5.5)-N(−2.0) | | | |
| C > N: C-terminal side will be inside | | | |
| >>> membrane topology: type 1b (cytoplasmic tail | | | |
| 449 to 586) | | | |
| MITDISC: discrimination of mitochondrial targeting seq | | | |
| R content: | 0 | Hyd Moment(75): | 2.21 |
| Hyd Moment (95): | 0.86 | G content: | 4 |
| D/E content: | 2 | S/T content: | 2 |
| Score: −9.50 | | | |

```
Gavel: prediction of cleavage sites for mitochondrial
preseq cleavage site motif not found
NUCDISC: discrimination of nuclear localization signals
pat4: KRRH (3) at 472
pat7: PETRRKT (4) at 203
bipartite: none
content of basic residues: 9.0%
NLS Score: 0.06
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: too long tail
Dileucine motif in the tail: found
LL at 455
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 70.6
COIL: Lupas's algorithm to detect coiled-coil regions
total: 0 residues
Final Results (k = 9/23):
34.8%: nuclear
26.1%: cytoplasmic
17.4%: mitochondrial
  8.7%: vesicles of secretory system
  4.3%: vacuolar
  4.3%: peroxisomal
  4.3%: endoplasmic reticulum
>> prediction for CG162855-01 is nuc (k = 23)
```

A search of the NOV19a protein against the Geneseq database, a proprietary database that contains sequences published in patents and patent publication, yielded several homologous proteins shown in Table 19C.

TABLE 19C
Geneseq Results for NOV19a

| | | | Identities/ | |
|---|---|---|---|---|
| | | | Similarities for | |
| Geneseq | Protein/Organism/Length | NOV19a Residues/ | the Matched | Expect |
| Identifier | [Patent #, Date) | Match Residues | Region | Value |
| AAM48908 | Human neurolignin family | 41 . . . 586 | 545/546 (99%) | 0.0 |
| | member 46980 protein - | 271 . . . 816 | 545/546 (99%) | |
| | Homo sapiens, 816 aa. | | | |
| | [WO200194563-A2, | | | |
| | 13 DEC. 2001] | | | |
| AAB33427 | Human PRO701 protein | 41 . . . 586 | 538/546 (98%) | 0.0 |
| | UNQ365 SEQ ID NO: 67 - | 272 . . . 816 | 539/546 (98%) | |
| | Homo sapiens, 816 aa. | | | |
| | [WO200053758-A2, | | | |
| | 14 SEP. 2000] | | | |
| AAB44296 | Human PRO701 (UNQ365) | 41 . . . 586 | 538/546 (98%) | 0.0 |
| | protein sequence SEQ ID | 272 . . . 816 | 539/546 (98%) | |
| | NO: 375 - Homo sapiens, 816 | | | |
| | aa. [WO200053756-A2, | | | |
| | 14 SEP. 2000] | | | |
| AAY41740 | Human PRO701 protein | 41 . . . 586 | 538/546 (98%) | 0.0 |
| | sequence - Homo sapiens, | 272 . . . 816 | 539/546 (98%) | |
| | 816 aa. [WO9946281-A2, | | | |
| | 16 SEP. 1999] | | | |
| AAB94127 | Human protein sequence | 78 . . . 586 | 502/509 (98%) | 0.0 |
| | SEQ ID NO: 14381 - Homo | 1 . . . 509 | 503/509 (98%) | |
| | sapiens, 509 aa. | | | |
| | [EP1074617-A2, | | | |
| | 07 FEB. 2001] | | | |

In a BLAST search of public sequence datbases, the NOV19a protein was found to have homology to the proteins shown in the BLASTP data in Table 19D.

TABLE 19D
Public BLASTP Results for NOV19a

| | | | Identities/ | |
|---|---|---|---|---|
| Protein | | | Similarities for | |
| Accession | | NOV19a Residues/ | the Matched | Expect |
| Number | Protein/Organism/Length | Match Residues | Portion | Value |
| Q8NFZ3 | Neuroligin Y - Homo sapiens | 41 . . . 586 | 545/546 (99%) | 0.0 |
| | (Human), 816 aa. | 271 . . . 816 | 545/546 (99%) | |
| Q9Y2F8 | Hypothetical protein | 41 . . . 586 | 545/546 (99%) | 0.0 |
| | KIAA0951 - Homo sapiens | 103 . . . 648 | 545/546 (99%) | |
| | (Human), 648 aa. | | | |
| Q8N0W4 | Neuroligin X - Homo sapiens | 41 . . . 586 | 539/546 (98%) | 0.0 |
| | (Human), 816 aa. | 271 . . . 816 | 540/546 (98%) | |
| Q9ULG0 | Hypothetical protein | 41 . . . 586 | 539/546 (98%) | 0.0 |
| | KIAA1260 - Homo sapiens | 272 . . . 817 | 540/546 (98%) | |
| | (Human), 817 aa (fragment). | | | |
| Q9NZ94 | Neuroligin 3 isoform - Homo | 41 . . . 586 | 413/547 (75%) | 0.0 |
| | sapiens (Human), 828 aa. | 285 . . . 828 | 468/547 (85%) | |

PFam analysis predicts that the NOV19a protein contains the domains shown in the Table 19E.

TABLE 19E
Domain Analysis of NOV19a

| | | Identities/ | |
|---|---|---|---|
| | NOV19a | Similarities for | |
| Pfam | Match | the Matched | Expect |
| Domain | Region | Region | Value |
| COesterase | 1 . . . 41 | 26/47 (55%) | 2.7e−11 |
| | | 34/47 (72%) | |
| COesterase | 42 . . . 360 | 97/361 (27%) | 4.8e−45 |
| | | 233/361 (65%) | |

### Example 20

The NOV20 clone was analyzed, and the nucleotide and encoded polypeptide sequences are shown in Table 20A.

TABLE 20A
NOV20 Sequence Analysis

| | |
|---|---|
| SEQ ID NO: 143 | 994 bp |

| | |
|---|---|
| NOV20a, | ATTGAAAATGCGTAAATTGGAAGGCAAGTTCTGAAATTAAACGTTGTACTTTGGCCTGATGTTCTGA |
| CG163937-01 | |
| DNA Sequence | CCTTTAAGGAAGCAAGAGTTTGTAAACTTCCAAATATTTACTATTCTGAACTGCCGTGTAAACCTGA |
| | CGTATTCCCAAGTCAACATACCAGTATACCAATAGGATGTGAATAATGTGTGTGTTGAGTTTAAAAC |
| | CATAGCAGTTTTGCTCTGGCAAGTAATGAAAGCGTTCTCGCTTCCTGAGTGTGAGCTCCAGCAGACT |
| | GCAGAGTGGCCAGTCCACAGTTGTAGCCTGACTTCAGTGAGTTCTGATGTGTGCTTTTTGCAAATAC |
| | ATGTTCTCAGAACAGTGAGATCATCCAGCAGTGGCCTGGACTGCACTCACATAAAAATCATGAGACA |
| | GCCATGGCTACTTGTTTCTGTAATACATGCATGTGTGTTTTTTAAAACCTATGATAGGCCTCTGATT |
| | CTGCAGCTGCAACTTTTATGGAATGTTTTCCTTCTCCACATCTCATGTGATGCTCTTATTACAGGAC |
| | ACAGCATTGTTGGTTTTGCCATGTACTATTTTACCTATGACCCGTGGATTGGCAAGTTATTGTATCT |
| | TGAGGACTTCTTCGTGATGAGTGATTATAGAGGCTTTGGCATAGGATCAGAAATTCTGAAGAATCTA |
| | AGCCAGGTTGCAATGAGGTGTCGCTGCAGCAGCATGCACTTCTTGGTAGCAGAATGGAATGAACCAT |
| | CCATCAACTTCTATAAAAGAAGAGGTGCTTCTGATCTGTCCAGTGAAGAGGGTTGGAGACTGTTCAA |
| | GATCGACAAGGAGTACTTGCTAAAAATGGCAACAGAGGAGTGAGGAGTGCTGCTGTAGATGACAACC |
| | TCCATTCTATTTTAGAATAAATTCCCAATTCTCTTGCTTTCTATGCTGTTGTAGTGAAATAATAGAA |
| | TGAGCACCCATTCCATAGCTTTATTACCAGTGGGCGTTGTTGCATGTTTGAACATG |

| | | |
|---|---|---|
| ORF Start: ATG at 227 | | ORF Stop: TGA at 845 |
| SEQ ID NO: 144 | 206 aa | MW at 23873.5 kD |

| | |
|---|---|
| NOV20a, | MKAFSLPECELQQTAEWPVHSCSLTSVSSDVCFLQIHVLRTVRSSSSGLDCTHIKIMRQPWLLVSVI |
| CG163937-01 | |
| Protein Sequence | HACVFFKTYDRPLILQLQLLWNVFLLHISCDALITGHSIVGFAMYYFTYDPWIGKLLYLEDFFVMSD |
| | YRGFGIGSEILKNLSQVAMRCRCSSMHFLVAEWNEPSINFYKRRGASDLSSEEGWRLFKIDKEYLLK |
| | MATEE |

Further analysis of the NOV20a protein yielded the following properties shown in Table 20B.

TABLE 20B
Protein Sequence Properties NOV20a

| SignalP | | | | |
|---|---|---|---|---|
| analysis: | Cleavage site between residues 12 and 13 | | | |
| PSORT II | PSG: a new signal peptide prediction method | | | |
| analysis: | N-region: length 10; pos.chg 1; neg.chg 2 | | | |
| | H-region: length 5; peak value 0.00 | | | |
| | PSG score: −4.40 | | | |
| | GvH: von Heijne's method for signal seq. recognition | | | |
| | GvH score (threshold: −2.1): −3.95 | | | |
| | possible cleavage site: between 29 and 30 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 1.70 (at 56) | | | |
| | ALOM score: −0.16 (number of TMSs: 0) | | | |
| | MITDISC: discrimination of mitochondrial targeting seq | | | |
| | R content: | 0 | Hyd Moment (75): | 7.74 |
| | Hyd Moment (95): | 1.81 | G content: | 0 |
| | D/E content: | 2 | S/T content: | 1 |

Score: −7.02
Gavel: prediction of cleavage sites for mitochondrial
preseq cleavage site motif not found
NUCDISC: discrimination of nuclear localization signals
pat4: none
pat7: none
bipartite: none
content of basic residues: 9.2%
NLS Score: −0.47
KDEL: ER retention motif in the C-terminus: none
ER Membrane Retention Signals: none
SKL: peroxisomal targeting signal in the C-terminus:
none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi:
none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs:
none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
Final Results (k = 9/23):
34.8%: cytoplasmic
34.8%: mitochondrial
30.4%: nuclear
>> prediction for CG163937-01 is cyt (k = 23)

A search of the NOV20a protein against the Geneseq database, a proprietary database that contains sequences published in patents and patent publication, yielded several homologous proteins shown in Table 20C.

TABLE 20C
Geneseq Results for NOV20a

| | | | Identities/ | | |
|---|---|---|---|---|---|
| | | | Similarities for | | |
| Geneseq | Protein/Organism/Length | NOV20a Residues/ | the Matched | | Expect |
| Identifier | [Patent #, Date] | Match Residues | Region | | Value |
| ABB57094 | Mouse ischaemic condition | 103 . . . 206 | 102/104 | (98%) | 2e−56 |
| | related protein sequence SEQ | 68 . . . 171 | 103/104 | (98%) | |
| | ID NO: 207 - Mus musculus, | | | | |
| | 171 aa. [WO200188188-A2, | | | | |
| | 22 NOV. 2001] | | | | |
| AAU30048 | Novel human secreted | 103 . . . 193 | 81/94 | (86%) | 6e−40 |
| | protein #539 - Homo sapiens, | 102 . . . 195 | 85/94 | (90%) | |
| | 218 aa. [WO200179449-A2, | | | | |
| | 25 OCT. 2001] | | | | |
| AAB44145 | Human cancer associated | 103 . . . 162 | 60/60 | (100%) | 2e−30 |
| | protein sequence SEQ ID | 27 . . . 86 | 60/60 | (100%) | |
| | NO: 1590 - Homo sapiens, 92 | | | | |
| | aa. [WO200055350-A1, | | | | |
| | 21 SEP. 2000] | | | | |
| ABP62823 | Human polypeptide SEQ ID | 101 . . . 203 | 44/103 | (42%) | 2e−21 |
| | NO 260 - Homo sapiens, 249 | 145 . . . 247 | 65/103 | (62%) | |
| | aa. [WO200218424-A2, | | | | |
| | 07 MAR. 2002] | | | | |
| AAW58394 | Human spermidine/spermine | 101 . . . 203 | 44/103 | (42%) | 2e−21 |
| | N1-acetyltransferase - Homo | 66 . . . 168 | 65/103 | (62%) | |
| | sapiens, 170 aa. | | | | |
| | [WO9818938-A1, | | | | |
| | 07 MAY 1998] | | | | |

In a BLAST search of public sequence datbases, the NOV20a protein was found to have homology to the proteins shown in the BLASTP data in Table 20D.

TABLE 20D
Public BLASTP Results for NOV20a

| Protein | | | Identities/ | |
|---|---|---|---|---|
| Accession | | NOV20a Residues/ | Similarities for the | Expect |
| Number | Protein/Organism/Length | Match Residues | Matched Portion | Value |
| JH0783 | diamine N-acetyltransferase | 103 . . . 206 | 104/104 (100%) | 5e−57 |
| | (EC 2.3.1.57) - human, 171 | 68 . . . 171 | 104/104 (100%) | |
| | aa. | | | |
| P21673 | Diamine acetyltransferase | 103 . . . 206 | 104/104 (100%) | 5e−57 |
| | (EC 2.3.1.57) | 68 . . . 171 | 104/104 (100%) | |
| | (Spermidine/spermine N(1)- | | | |
| | acetyltransferase) (SSAT) | | | |
| | (Putrescine acetyltrans- | | | |
| | ferase) - Homo sapiens | | | |
| | (Human), 171 aa. | | | |
| Q9JHW6 | Spermidine/spermine | 103 . . . 206 | 102/104 (98%) | 5e−56 |
| | N1-acetyltransferase - | 68 . . . 171 | 103/104 (98%) | |
| | Cricetulus griseus (Chinese | | | |
| | hamster), 171 aa. | | | |
| Q28999 | Diamine acetyltransferase | 103 . . . 206 | 102/104 (98%) | 5e−56 |
| | (EC 2.3.1.57) | 68 . . . 171 | 103/104 (98%) | |
| | (Spermidine/spermine N(1)- | | | |
| | acetyltransferase) (SSAT) | | | |
| | (Putrescine acetyltrans- | | | |
| | ferase) - Sus scrofa (Pig), | | | |
| | 171 aa. | | | |
| P49431 | Spermidine/spermine | 103 . . . 206 | 102/104 (98%) | 5e−56 |
| | N(1)-acetyltransferase (EC | 68 . . . 171 | 103/104 (98%) | |
| | 2.3. 1.57) (Diamine | | | |
| | acetyltransferase) (SSAT) | | | |
| | (Putrescine acetyltrans- | | | |
| | ferase) - Mus saxicola (Spiny | | | |
| | mouse), 171 aa. | | | |

PFam analysis predicts that the NOV20a protein contains the domains shown in the Table 20E.

TABLE 20E
Domain Analysis of NOV20a

| | | Identities/ | |
|---|---|---|---|
| | | Similarities | |
| | | for the | |
| | NOV20a Match | Matched | Expect |
| Pfam Domain | Region | Region | Value |
| Acetyltransf | 98 . . . 181 | 22/85 (26%) | 9.6e−16 |
| | | 59/85 (69%) | |

### Example 21

The NOV21 clone was analyzed, and the nucleotide and encoded polypeptide sequences are shown in Table 21A.

TABLE 21A
NOV21 Sequence Analysis

| | |
|---|---|
| SEQ ID NO: 145 | 744 bp |

| | |
|---|---|
| NOV21a, | AAATGCAGCCATTCCTCCTCCTGTTGGCCTTTCTTCTGACCCCTGGGGCTGGGACAGAGGAGATCAT |
| CG164449-02 | |
| DNA Sequence | CGGGGGCCATGAGGCCAAGCCCCACTCCCGCCCCTACATGGCCTTTGTTCAGTTTCTGCAAGAGAAG |
| | AGTCGGAAGAGGTGTGGCGGCATCCTAGTGAGAAAGGACTTTGTGCTGACAGCTGCTCACTGCCAGG |
| | GAAGCTCCATAAATGTCACCTTGGGGGCCCACAATATCAAGGAACAGGAGCGGACCCAGCAGTTTAT |
| | CCCTGTGAAAAGACCCATCCCCCATCCAGCCTATAATCCTAAGAACTTCTCCAACGACATCATGCTA |
| | CTGCAGCTGGAGAGAAAGGCCAAGTGGACCACAGCTGTGCGGCCTCTCAGGCTACCTAGCAGCAAGG |
| | CCCAGGTGAAGCCAGGGCAGCTGTGCAGTGTGGCTGGCTGGGGTTATGTCTCAATGAGCACTTTAGC |
| | AACCACACTGCAGGAAGTGTTGCTGACAGTGCAGAAGGACTGCCAGTGTGAACGTCTCTTCCATGGC |
| | AATTACAGCAGAGCCACTGAGATTTGTGTGGGGGATCCAAAGAAGACACAGACCGGTTTCAAGGGGG |
| | ACTCCGGGGGGCCCCTCGTGTGTAAGGACGTAGCCCAAGGTATTCTCTCCTATGGAAACAAAAAAGG |
| | GACACCTCCAGGAGTCTACATCAAGGTCTCACACTTCCTGCCCTGGATAAAGAGAACAATGAAGCGC |
| | CTCTAAC |

| | | |
|---|---|---|
| ORF Start: ATG at 3 | | ORF Stop: TAA at 741 |
| SEQ ID NO: 146 | 246 aa | MW at 27314.7 kD |

| | |
|---|---|
| NOV21a, | MQPFLLLLAFLLTPGAGTEEIIGGHEAKPHSRPYMAFVQFLQEKSRKRCGGILVRKDFVLTAAHCQG |
| CG164449-02 | |
| Protein Sequence | SSINVTLGAHNIKEQERTQQFIPVKRPIPHPAYNPKNFSNDIMLLQLERKAKWTTAVRPLRLPSSKA |
| | QVKPGQLCSVAGWGYVSMSTLATTLQEVLLTVQKDCQCERLFHGNYSRATEICVGDPKKTQTGFKGD |
| | SGGPLVCKDVAQGILSYGNKKGTPPGVYIKVSHFLPWIKRTMKRL |

| | |
|---|---|
| SEQ ID NO: 147 | 576 bp |

| | |
|---|---|
| NOV21b, | CTGACCTGGGCAGCCTTCCTGAGAAAATGCAGCCATTCCTCCTCCTGTTGGCCTTTCTTCTCACCCC |
| CG164449-01 | |
| DNA Sequence | TGGGGCTGGGACAGAGGAGATCATCGGGGGCCATGAGGCCAAGCCCCACTCCCGCCCCTACATGGCC |
| | TTTGTTCAGTTTCTGCAAGAGAAGAGTCGGAAGAGGTGTGGCGGCATCCTAGTGAGAAAGGACTTTG |
| | TGCTGACAGCTGCTCACTGCCAGGTAAGCTCCATAAATGTCACCTTGGGGGCCCACAATATCAAGGA |
| | ACAGGAGCGGACCCAGCAGTTTATCCCTGTGAAAAGACCCATCCCCCATCCAGCCTATAATCCTAAG |
| | AACTTCTCCAACGACATCATGCTACTGCAGCTGGAGAGAAAGGCCAAGTGGACCACAGCTGTGCGGC |
| | CTCTCAGGCTACCTAGCAGCAAGGCCCAGGGGGACTCCGGGGGGCCCCTCGTGTGTAAGGACGTAGC |
| | CCAAGGTATTCTCTCCTATGGAAACAAAAAAGGGACACCTCCAGGAGTCTACATCAAGGTCTCACAC |
| | TTCCTGCCCTGGATAAAGAGAACAATGAAGCGCCTCTAAC |

| | | |
|---|---|---|
| ORF Start: ATG at 27 | | ORF Stop: TAA at 573 |
| SEQ ID NO: 148 | 182 aa | MW at 20350.7kD |

| | |
|---|---|
| NOV21b, | MQPFLLLLAFLLTPGAGTEEIIGGHEAKPHSRPYMAFVQFLQEKSRKRCGGILVRKDFVLTAAHCQV |
| CG164449-01 | |
| Protein Sequence | SSINVTLGAHNIKEQERTQQFIPVKRPIPHPAYNPKNFSNDIMLLQLERKAKWTTAVRPLRLPSSKA |
| | QGDSGGPLVCKDVAQGILSYGNKKGTPPGVYIKVSHFLPWIKRTMKRL |

Sequence comparison of the above protein sequences yields the following sequence relationships shown in Table 21B.

Further analysis of the NOV21 a protein yielded the following properties shown in Table 21C.

TABLE 21B
Comparison of NOV21a against NOV21b.

| | NOV21a Residues/ | Identities/Similarities |
|---|---|---|
| Protein Sequence | Match Residues | for the Matched Region |
| NOV21b | 1 . . . 166 | 141/168 (83%) |
| | 1 . . . 168 | 144/168 (84%) |

TABLE 21C
Protein Sequence Properties NOV21a

| SignalP | | |
|---|---|---|
| analysis: | Cleavage site between residues 18 and 19 | |
| PSORT II | PSG: | a new signal peptide prediction method |
| analysis: | | N-region: length 0; pos.chg 0; neg.chg 0 |
| | | H-region: length 18; peak value 12.03 |
| | | PSG score: 7.62 |
| | GvH: | von Heijne's method for signal seq. recognition |
| | | GvH score (threshold: -2.1): 3.30 |
| | | possible cleavage site: between 18 and 19 |
| | >>> Seems to have a cleavable signal peptide (1 to 18) | |
| | ALOM: | Klein et al's method for TM region allocation |
| | | Init position for calculation: 19 |
| | | Tentative number of TMS(s) for the threshold 0.5: 0 |
| | | number of TMS(s) . . . fixed |
| | | PERIPHERAL Likelihood = 3.61 (at 150) |
| | | ALOM score: 3.61 (number of TMSs: 0) |
| | MTOP: | Prediction of membrane topology (Hartmann et al.) |
| | | Center position for calculation: 9 |
| | | Charge difference: −1.0 C(0.0) − N(1.0) |
| | | N >= C: N-terminal side will be inside |

MITDISC: discrimination of mitochondrial targeting seq

| | | | |
|---|---|---|---|
| R content: | 0 | Hyd Moment | 2.63 |
| Hyd Moment (95): | 5.02 | (75): | |
| D/E content: | 1 | G content: | 2 |
| Score: | −5.96 | S/T content: | 2 |

| Gavel: | prediction of cleavage sites for mitochondrial preseq |
|---|---|
| | cleavage site motif not found |
| NUCDISC: discrimination of nuclear localization signals | |
| | pat4: none |
| | pat7: none |
| | bipartite: none |
| | content of basic residues: 13.8% |
| | NLS Score: −0.47 |
| KDEL: | ER retention motif in the C-terminus: none |
| ER Membrane Retention Signals: | |
| | KKXX-like motif in the C-terminus: TMKR |
| SKL: | peroxisomal targeting signal in the C-terminus: none |
| PTS2: | 2nd peroxisomal targeting signal: none |
| VAC: | possible vacuolar targeting motif: none |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| discrimination | |
| | Prediction: cytoplasmic |
| | Reliability: 70.6 |
| COIL: | Lupas's algorithm to detect coiled-coil regions |
| | total: 0 residues |

----------------------------------
Final Results (k = 9/23):

44.4%: endoplasmic reticulum
33.3%: extracellular, including cell wall
11.1%: mitochondrial
11.1%: vacuolar

>> prediction for CG164449-02 is end (k = 9)

A search of the NOV21a protein against the Geneseq database, a proprietary database that contains sequences published in patents and patent publication, yielded several homologous proteins shown in Table 21D.

TABLE 21D
Geneseq Results for NOV21a

| Geneseq | Protein/Organism/Length | NOV21a Residues/ | Identities/Similarities | Expect |
|---|---|---|---|---|
| Identifier | [Patent #, Date] | Match Residues | for the Matched Region | Value |
| AAB58142 | Lung cancer associated | 1 . . . 246 | 246/246 (100%) | e−145 |
| | polypeptide sequence SEQ | 33 . . . 278 | 246/246 (100%) | |
| | ID 480 - Homo sapiens, 278 | | | |
| | aa. [WO200055180-A2, | | | |
| | 21-SEP-2000] | | | |
| AAR13253 | Human Cytotoxic Cell | 1 . . . 246 | 246/246 (100%) | e−145 |
| | Protease-X - Homo sapiens, | 1 . . . 246 | 246/246 (100%) | |
| | 246 aa. [WO9110685-A, | | | |
| | 25-JUL-1991] | | | |
| AAW84158 | A human serine protease | 1 . . . 245 | 194/246 (78%) | e−108 |
| | precursor (HSPP) protein - | 1 . . . 246 | 208/246 (83%) | |
| | Homo sapiens, 247 aa. | | | |
| | [WO9850424-A2, | | | |
| | 12-NOV-1998] | | | |
| AAE24317 | Human granzyme B (grB) | 1 . . . 245 | 176/246 (71%) | 2e−97 |
| | protein - Homo sapiens, 247 | 1 . . . 246 | 194/246 (78%) | |
| | aa. [WO200234910-A2, | | | |
| | 02-MAY-2002] | | | |
| AAR27722 | Human Granzyme B in | 21 . . . 145 | 159/226 (70%) | 2e−87 |
| | vector 3038 - Mus musculus, | 1 . . . 226 | 177/226 (77%) | |
| | 227 aa. [WO9216644-A, | | | |
| | 01-OCT-1992] | | | |

In a BLAST search of public sequence datbases, the NOV21a protein was found to have homology to the proteins shown in the BLASTP data in Table 21E.

TABLE 21E
Public BLASTP Results for NOV21a

| Protein | | | | |
|---|---|---|---|---|
| Accession | | NOV21a Residues/ | Identities/Similarities | Expect |
| Number | Protein/Organism/Length | Match Residues | for the Matched Portion | Value |
| P20718 | Granzyme H precursor (EC 3.4.21.-) | 1 . . . 246 | 246/246 (100%) | e−144 |
| | (Cytotoxic T-lymphocyte proteinase) | 1 . . . 246 | 246/246 (100%) | |
| | (Cathepsin G-like 2) (CTSGL2) | | | |
| | (CCP-X) (Cytotoxic serine protease-C) | | | |
| | (CSP-C) - Homo sapiens | | | |
| | (Human), 246 aa. | | | |
| CAD48710 | Sequence 7 from Patent | 1 . . . 245 | 176/246 (71%) | 5e−97 |
| | WO0234910 - Homo sapiens | 1 . . . 246 | 194/246 (78%) | |
| | (Human), 247 aa. | | | |
| A61021 | granzyme B (EC 3.4.21.79) precursor | 1 . . . 245 | 175/246 (71%) | 2e−96 |
| | [validated] - human, 281 aa. | 35 . . . 280 | 194/246 (78%) | |
| Q8N1D2 | Granzyme B (Granzyme 2, cytotoxic | 1 . . . 245 | 175/246 (71%) | 2e−96 |
| | T-lymphocyte-associated serine | 1 . . . 246 | 194/246 (78%) | |
| | esterase 1) - Homo | | | |
| | sapiens (Human), 247 aa. | | | |
| P10144 | Granzyme B precursor (EC 3.4.21.79) | 1 . . . 245 | 175/246 (71%) | 2e−96 |
| | (T-cell serine protease 1-3E) (Cytotoxic | 1 . . . 246 | 194/246 (78%) | |
| | T-lymphocyte proteinase 2) | | | |
| | (Lymphocyte protease) (SECT) | | | |
| | (Granzyme 2) (Cathepsin G-like 1) | | | |
| | (CTSGL1) (CTLA-1) (Fragmentin 2) | | | |
| | (Human lymphocyte protein) (HLP) | | | |
| | (C11) - Homo sapiens (Human), 247 aa. | | | |

PFam analysis predicts that the NOV21a protein contains the domains shown in the Table 21F.

TABLE 21F
Domain Analysis of NOV21a

| | NOV21a | Identities/Similarities | Expect |
|---|---|---|---|
| Pfam Domain | Match Region | for the Matched Region | Value |
| trypsin | 21 . . . 239 | 91/262 (35%) | 3.4e−79 |
| | | 184/262 (70%) | |

### Example 22

The NOV22 clone was analyzed, and the nucleotide and encoded polypeptide sequences are shown in Table 22A.

TABLE 22A
NOV22 Sequence Analysis

| | |
|---|---|
| SEQ ID NO: 149 | 2205 bp |

| | |
|---|---|
| NOV22a, | ATGTGGGGGCTCCTGCTCGCCCTGGCCGCCTTCGCGCCGGCCGTCGGCCCGGCTCTGGGGGCGCCCA |
| CG54007-06 | |
| DNA Sequence | GGAACTCGGTGCTGGGCCTCGCGCAGCCCGGGACCACCAAGGTCCCAGGCTCGACCCCGGCCCTGCA |
| | TAGCAGCCCGGCACAGCCGCCGGCGGAGACAGCTAACGGGACCTCAGAACAGCATGTCCGGATTCGT |
| | GTCATCAAGAAGAAAAAGGTCATTATGAAGAAGCGGAAGAAGCTAACTCTAACTCGCCCCACCCCAC |
| | TGGTGACTGCCGGGCCCCTTGTGACCCCCACTCCAGCAGGGACCCTCGACCCCGCTGAGAAACAAGA |
| | AACAGGCTGTCCTCCTTTGGGTCTGGAGTCCCTGCGAGTTTCAGATAGCCGGCTTGAGGCATCCAGC |
| | AGCCAGTCCTTTGGTCTTGGACCACACCGAGGACGGCTCAACATTCAGTCAGGCCTGGAGGACGGCG |
| | ATCTATATGATGGAGCCTGGTGTGCTGAGGAGCAGGACGCCGATCCATGGTTTCAGGTGGACGCTGG |
| | GCACCCCACCCGCTTCTCGGGTGTTATCACACAGGGCAGGAACTCTGTCTGGAGGTATGACTGGGTC |
| | ACATCATACAAGGTCCAGTTCAGCAATGACAGTCGGACCTGGTGGGGAAGTAGGAACCACAGCAGTG |
| | GGATGGACGCAGTATTTCCTGCCAATTCAGACCCAGAAACTCCAGTGCTGAACCTCCTGCCGGAGCC |
| | CCAGGTGGCCCGCTTCATTCGCCTGCTGCCCCAGACCTGGCTCCAGGGAGGCGCGCCTTGCCTCCGG |
| | GCAGAGATCCTGGCCTGCCCAGTCTCAGACCCCAATGACCTATTCCTTGAGGCCCCTGCGTCGGGAT |
| | CCTCTGACCCTCTAGACTTTCAGCATCACAATTACAAGGCCATGAGGAAGCTGATGAAGCAGGTACA |
| | AGAGCAATGCCCCAACATCACCCGCATCTACAGCATTGGGAAGAGCTACCAGGGCCTGAAGCTGTAT |
| | GTGATGGAAATGTCGGACAAGCCTGGGGAGCATGAGCTGGGGGAGCCTGAGGTGCGCTACGTGGCTG |
| | GCATGCATGGGAACGAGGCCCTGGGGCGGGAGTTGCTTCTGCTCCTGATGCAGTTCCTGTGCCATGA |
| | GTTCCTGCGAGGGAACCCACGGGTGACCCGGCTGCTCTCTGAGATGCGCATTCACCTGCTGCCCTCC |
| | ATGAACCCTGATGGCTATGAGATCGCCTACCACCGGGGTTCAGAGCTGGTGGGCTGGGCCGAGGGCC |
| | GCTGGAACAACCAGAGCATCGATCTTAACCATAATTTTGCTGACCTCAACACACCACTGTGGGAAGC |
| | ACAGGACGATGGGAAGGTGCCCCACATCGTCCCCAACCATCACCTGCCATTGCCCACTTACTACACC |
| | CTGCCCAATGCCACCGTGGCTCCTGAAACGCGGGCAGTAATCAAGTGGATGAAGCGGATCCCCTTTG |
| | TGCTAAGTGCCAACCTCCACGGGGGTGAGCTCGTGGTGTCCTACCCATTCGACATGACTCGCACCCC |
| | GTGGGCTGCCCGCGAGCTCACGCCCACACCAGATGATGCTGTGTTTCGCTGGCTCAGCACTGTCTAT |
| | GCTGGCAGTAATCTGGCCATGCAGGACACCAGCCGCCGACCCTGCCACAGCCAGGACTTCTCCGTGC |
| | ACGGCAACATCATCAACGGGGCTGACTGGCACACGGTCCCCGGGAGCATGAATGACTTCAGCTACCT |
| | ACACACCAACTGCTTTGAGGTCACTGTGGAGCTGTCCTGTGACAAGTTCCCTCACGAGAATGAATTG |
| | CCCCAGGAGTGGGAGAACAACAAAGACGCCCTCCTCACCTACCTGGAGCAGGTGCGCATGGGCATTG |
| | CAGGAGTGGTGAGGGACAAGGACACGGAGCTTGGGATTGCTGACGCTGTCATTGCCGTGGATGGGAT |
| | TAACCATGACGTGACCACGGCGTGGGGCGGGGATTATTGGCGTCTGCTGACCCCAGGGGACTACATG |
| | GTGACTGCCAGTGCCGAGGGCTACCATTCAGTGACACGGAACTGTCGGGTCACCTTTGAAGAGGGCC |
| | CCTTCCCCTGCAATTTCGTGCTCACCAAGACTCCCAAACAGAGGCTGCGCGAGCTGCTGGCAGCTGG |
| | GGCCAAGGTGCCCCCGGACCTTCGCAGGCGCCTGGAGCGGCTAAGGGGACAGAAGGATTGA |

| | | |
|---|---|---|
| ORF Start: ATG at 1 | | ORF Stop: TGA at 2203 |
| SEQ ID NO: 150 | 734 aa | MW at 81666.8 kD |

| | |
|---|---|
| NOV22a, | MWGLLLALAAFAPAVGPALGAPRNSVLGLAQPGTTKVPGSTPALHSSPAQPPAETANGTSEQHVRIR |
| CG54007-06 | |
| Protein Sequence | VIKKKKVIMKKRKKLTLTRPTPLVTAGPLVTPTPAGTLDPAEKQETGCPPLGLESLRVSDSRLEASS |
| | SQSFGLGPHRGRLNIQSGLEDGDLYDGAWCAEEQDADPWFQVDAGHPTRFSGVITQGRNSVWRYDWV |
| | TSYKVQFSNDSRTWWGSRNHSSGMDAVFPANSDPETPVLNLLPEPQVARFIRLLPQTWLQGGAPCLR |
| | AEILACPVSDPNDLFLEAPASGSSDPLDFQHHNYKAMRKLMKQVQEQCPNITRIYSIGKSYQGLKLY |
| | VMEMSDKPGEHELGEPEVRYVAGMHGNEALGRELLLLLMQFLCHEFLRGNPRVTRLLSEMRIHLLPS |
| | MNPDGYEIAYHRGSELVGWAEGRWNNQSIDLNHNFADLNTPLWEAQDDGKVPHIVPNHHLPLPTYYT |
| | LPNATVAPETRAVIKWMKRIPFVLSANLHGGELVVSYPFDMTRTPWAARELTPTPDDAVFRWLSTVY |
| | AGSNLAMQDTSRRPCHSQDFSVHGNIINGADWHTVPGSMNDFSYLHTNCFEVTVELSCDKFPHENEL |
| | PQEWENNKDALLTYLEQVRMGIAGVVRDKDTELGIADAVIAVDGINHDVTTAWGGDYWRLLTPGDYM |
| | VTASAEGYHSVTRNCRVTFEEGPFPCNFVLTKTPKQRLRELLAAGAKVPPDLRRRLERLRGQKD |

| | |
|---|---|
| SEQ ID NO: 151 | 1725 bp |

| | |
|---|---|
| NOV22b, | ATGTGGGGGCTCCTGCTCGCCCTGGCCGCCTTCGCGCCGGCCGTCGGCCCGGCTCTGGGGGCGCCCA |
| CG54007-04 | |
| DNA Sequence | GGAACTCGGTGCTGGGCCTCGCGCAGCCCGGGACCACCAAGGTCCCAGGCTCGACCCCGGCCCTGCA |
| | TAGCAGCCCGGCACAGCCGCCGGCGGAGACAGCTAACGGGACCTCAGAACAGCATGTCCGGATTCGA |
| | GTCATCAAGAAGAAAAAGGTCATTATGAAGAAGCGGAAGAAGCTAACTCTAACTCGCCCCACCCCAC |
| | TGGTGACTGCCGGGCCCCTTGTGACCCCCACTCCAGCAGGGACCCTCGACCCCGCTGAGAAACAAGA |
| | AACAGGCTGTCCTCCTTTGGGTCTGGAGTCCCTGCGAGTTTCAGATAGCCGGCTTGAGGCATCCAGC |
| | AGCCAGTCCTTTGGTCTTGGACCACACCGAGGACGGCTCAACATTCAGTCAGGCCTGGAGGACGGCG |
| | ATCTATATGATGGAGCCTGGTGTGCTGAGGAGCAGGACGCCGATCCATGGTTTCAGGTGGACGCTGG |
| | GCACCCCACCCGCTTCTCGGGTGTTATCACACAGGGCAGGAACTCTGTCTGGAGGTATGACTGGGTC |
| | ACATCATACAAGGTCCAGTTCAGCAATGACAGTCGGACCTGGTGGGGAAGTAGGAACCACAGCAGTG |
| | GGATGGACGCAGTATTTCCTGCCAATTCAGACCCAGAAACTCCAGTGCTGAACCTCCTGCCGGAGCC |
| | CCAGGTGGCCCGCTTCATTCGCCTGCTGCCCCAGACCTGGCTCCAGGGAGGCGCGCCTTGCCTCCGG |
| | GCAGAGATCCTGGCCTGCCCAGTCTCAGACCCCAATGACCTATTCCTTGAGGCCCCTGCGTCGGGAT |
| | CCTCTGACCCTCTAGACTTTCAGCATCACAATTACAAGGCCATGAGGAAGCTGATGAAGCAGGTACA |
| | AGAGCAATGCCCCAACATCACCCGCATCTACAGCATTGGGAAGAGCTACCAGGGCCTGAAGCTGTAT |
| | GTGATGGAAATGTCGGACAAGCCTGGGGAGCATGAGCTGGGGGAGCCTGAGGTGCGCTACGTGGCTG |
| | GCATGCATGGGAACGAGGCCCTGGGGCGGGAGTTGCTTCTGCTCCTGATGCAGTTCCTGTGCCATGA |
| | GTTCCTGCGAGGGAACCCACGGGTGACCCGGCTGCTCTCTGAGATGCGCATTCACCTGCTGCCCTCC |
| | ATGAACCCTGATGGCTATGAGATCGCCTACCACCGGGGTTCAGAGCTGGTGGGCTGGGCCGAGGGCC |
| | GCTGGAACAACCAGAGCATCGATCTTAACCATAATTTTGCTGACCTCAACACACCACTGTGGGAAGC |
| | ACAGGACGATGGGAAGGTGCCCCACATCGTCCCCAACCATCACCTGCCATTGCCCACTTACTACACC |
| | CTGCCCAATGCCACCGTGGCTCCTGAAACGCGGGCAGTAATCAAGTGGATGAAGCGGATCCCCTTTG |
| | CTGCCCAATGCCACCGTGGCTCCTGAAACGCGGGCAGTAATCAAGTGGATGAAGCGGATCCCCTTTG |
| | TGCTAAGTGCCAACCTCCACGGGGGTGAGCTCGTGGTGTCCTACCCATTCGACATGGTGACTGCCAG |
| | TGCCGAGGGCTACCATTCAGTGACACGGAACTGTCGGGTCACCTTTGAAGAGGGCCCCTTCCCCTGC |
| | AATTTCGTGCTCACCAAGACTCCCAAACAGAGGCTGCGCGAGCTGCTGGCAGCTGGGGCCAAGGTGC |
| | CCCCGGACCTTCGCAGGCGCCTGGAGCGGCTAAGGGGACAGAAGGATTGA |

| | | |
|---|---|---|
| ORF Start: ATG at 1 | | ORF Stop: TGA at 1723 |
| SEQ ID NO: 152 | 574 aa | MW at 63683.0 kD |

| | |
|---|---|
| NOV22b, | MWGLLLALAAFAPAVGPALGAPRNSVLGLAQPGTTKVPGSTPALHSSPAQPPAETANGTSEQHVRIR |
| CG54007-04 | |
| Protein Sequence | VIKKKKVIMKKRKKLTLTRPTPLVTAGPLVTPTPAGTLDPAEKQETGCPPLGLESLRVSDSRLEASS |
| | SQSFGLGPHRGRLNIQSGLEDGDLYDGAWCAEEQDADPWFQVDAGHPTRFSGVITQGRNSVWRYDWV |
| | TSYKVQFSNDSRTWWGSRNHSSGMDAVFPANSDPETPVLNLLPEPQVARFIRLLPQTWLQGGAPCLR |
| | AEILACPVSDPNDLFLEAPASGSSDPLDFQHHNYKAMRKLMKQVQEQCPNITRIYSIGKSYQGLKLY |
| | VMEMSDKPGEHELGEPEVRYVAGMHGNEALGRELLLLLMQFLCHEFLRGNPRVTRLLSEMRIHLLPS |
| | MNPDGYEIAYHRGSELVGWAEGRWNNQSIDLNHNFADLNTPLWEAQDDGKVPHIVPNHHLPLPTYYT |
| | LPNATVAPETRAVIKWMKRIPFVLSANLHGGELVVSYPFDMVTASAEGYHSVTRNCRVTFEEGPFPC |
| | NFVLTKTPKQRLRELLAAGAKVPPDLRRRLERLRGQKD |

| | |
|---|---|
| SEQ ID NO: 153 | 20190 bp |

| | |
|---|---|
| NOV22c, | ATGTGGGGGCTCCTGCTCGCCCTGGCCGCCTTCGCGCCGGCCGTCGGCCCGGCTCTGGGGGCGCCCA |
| CG54007-01 | |
| DNA Sequence | GGAACTCGGTGCTGGGCCTCGCGCAGCCCGGGACCACCAAGGTCCCAGGCTCGACCCCGGCCCTGCA |
| | TAGCAGCCCGGCACAGCCGCCGGCGGAGACAGCTAACGGGACCTCAGAACAGCATGTCCGGATTCGA |
| | GTCATCAAGAAGAAAAAGGTCATTATGAAGAAGCGGAAGAAGCTAACTCTAACTCGCCCCACCCCAC |
| | TGGTGACTGCCGGGCCCCTTGTGACCCCCACTCCAGCAGGGACCCTCGACCCCGCTGAGAAACAAGA |
| | AACAGGCTGTCCTCCTTTGGGTCTGGAGTCCCTGCGAGTTTCAGATAGCCGGCTTGAGGCATCCAGC |
| | AGCCAGTCCTTTGGTCTTGGACCACACCGAGGACGGCTCAACATTCAGTCAGGCCTGGAGGACGGCG |
| | ATCTATATGATGGAGCCTGGTGTGCTGAGGAGCAGGACGCCGATCCATGGTTTCAGGTGGACGCTGG |
| | GCACCCCACCCGCTTCTCGGGTGTTATCACACAGGGCAGGAACTCTGTCTGGAGGTATGACTGGGTC |
| | ACATCATACAAGGTCCAGTTCAGCAATGACAGTCGGACCTGGTGGGGAAGTAGGAACCACAGCAGTG |
| | GGATGGACGCAGTATTTCCTGCCAATTCAGACCCAGAAACTCCAGTGCTGAACCTCCTGCCGGAGCC |
| | CCAGGTGGCCCGCTTCATTCGCCTGCTGCCCCAGACCTGGCTCCAGGGAGGCGCGCCTTGCCTCCGG |
| | GCAGAGATCCTGGCCTGCCCAGTCTCAGACCCCAATGACCTATTCCTTGAGGCCCCTGCGTCGGGAT |
| | CCTCTGACCCTCTAGACTTTCAGCATCACAATTACAAGGCCATGAGGAAGCTGATGAAGCAGGTACA |
| | AGAGCAATGCCCCAACATCACCCGCATCTACAGCATTGGGAAGAGCTACCAGGGCCTGAAGCTGTAT |
| | GTGATGGAAATGTCGGACAAGCCTGGGGAGCATGAGCTGGGGGAGCCTGAGGTGCGCTACGTGGCTG |
| | GCATGCATGGGAACGAGGCCCTGGGGCGGGAGTTGCTTCTGCTCCTGATGCAGTTCCTGTGCCATGA |
| | GTTCCTGCGAGGGAACCCACGGGTGACCCGGCTGCTCTCTGAGATGCGCATTCACCTGCTGCCCTCC |
| | ATGAACCCTGATGGCTATGAGATCGCCTACCACCGGGGTTCAGAGCTGGTGGGCTGGGCCGAGGGCC |
| | GCTGGAACAACCAGAGCATCGATCTTAACCATAATTTTGCTGACCTCAACACACCACTGTGGGAAGC |
| | ACAGGACGATGGGAAGGTGCCCCACATCGTCCCCAACCATCACCTGCCATTGCCCACTTACTACACC |
| | CTGCCCAATGCCACCGTGGCTCCTGAAACGCGGGCACTAATCAAGTGGATGAAGCGGATCCCCTTTG |
| | TGCTAAGTGCCAACCTCCACGGGGGTGAGCTCGTGGTGTCCTACCCATTCGACATGACTCGCACCCC |
| | GTGGGCTGCCCGCGAGCTCACGCCCACACCAGATGATGCTGTGTTTCGCTGGCTCAGCACTGTCTAT |
| | GCTGGCAGTAATCTGGCCATGCAGGACACCAGCCGCCGACCCTGCCACAGCCAGGACTTCTCCGTGC |
| | ACGGCAACATCATCAACGGGGCTGACTGGCACACGGTCCCCGGGAGCATGAATGACTTCAGCTACCT |
| | ACACACCAACTGCTTTGAGGTCACTGTGGAGCTGTCCTGTGACAAGTTCCCTCACGAGAATGAATTG |
| | CCCCAGGAGTGGGAGAACAACAAAGACGCCCTCCTCACCTACCTGGAGCAGGTGCGCATGGGCATTG |
| | CAGGAGTGGTGAGGGACAAGGACACGGAGCTTGGGATTGCTGACGCTGTCATTGCCGTGGATGGGAT |
| | TAACCATGACGTGACCACGGCGTGGGGCGGGGATTATTGGCGTCTGCTGACCCCAGGGGACTACATG |
| | GTGACTGCCAGTGCCGAGGGCTACCATTCAGTGACACGGAACTGTCGGGTCACCTTTGAAGAGGGCC |
| | CCTTCCCCTGCAATTTCGTGCTCACCAAGACTCCCAAACAGAGGCTGCGCGAGCTGCTGGCAGCTGG |
| | GGCCAAGGTGCCCCCGGACCTTCGCAGGCGCCTGGAGCGGCTAAGGGGACAGAAGGATTGANNANTN |
| | CANNTTNANNNTNGNNANNTCTCACTTATAAATGGAAGCTGGCGGGACACGGTGGCTCACTCCTGTA |
| | ATCCCAACACTTTGGGAGGCTGAGGCGGGTGGATCACGAGGTCAGGAGATCGAGACCATCCTGACTA |
| | ACACGGTGAAACCCGTCTCTACTAAAAACACAAAAAATTAGCTGGGCGTGGTGGCGGCACCTGTAGT |
| | CCCAGCTACTCGGGAGGCTGAGGCAGGAGAATGGCATGAACCCAGGAGTCGGAGCTTGCAGTGAGCC |
| | GAGTTCACGCCACTGCATTCCAGCCTGGGCAACAGAGCGAGACTCTGTCTCAAAAAAAATAAATTAA |
| | ATAAAAATAAATAAATGGAAACTAAGCTGTGGGTATGCAAAGGCATACAGAATGGTATAATGGACAT |
| | TGGAGACTCAGAAGGAGGAGGGTAAGCGGGGGGTGACAGATAAAAAAAACTGCATGTTGCATACAAT |
| | GTACACTACTCGGGTGATGGGCGCTCTAAGATTTCAAACTTCACCACTATACAGTTCTCCCCTGTAA |
| | CCAAAAACCGCTGGTACCCCTAAAGCAATTGAAATAAAAATAGAAACTATGTTGTAGCCTGGATGAC |
| | ATAGCGAAAACTTGTCTCTTAAAAAAAAAAAAATGTGGCCGGGTGCAGTGGCTCACACCTGTAATCC |
| | CAGCACTTTGGGAGGCCCAAGGCGGGCAGATCACAAGGTCAGGAGATTGAGACCGTCCTGGCTAACA |
| | AGGTGAAACTCCATCTCTACTAAAAATACAAAAAATTAGCCGGGTGTGGTGGCACACGCCTGTAATC |
| | CCAGCTACTTGGGAGGCTGAGGCAGGAGAATCGCTTGAACCCAGAGGCGGAGGTTGCAGTGAGCCGA |
| | GATCGCACCACAGCACTCCAGCCTGGTGACAGAGTGAGATTTAGTCTCAAAAAAAAAAAAAAAAAAA |
| | AAAAAAAAAAGGTAGAAATTAGCTGAGCGTGGTGACACGTCCCAGATACTTGGGAGGCTGAGGTGGG |
| | AGGATCGCTTGAACCCAGGAGTTCCAGACTGCAGTGAGCTGTGATTACACTATTGCACTCCAGCCTA |
| | GGCTGTGGGAAAGAGAGTTTCTGGGGTGCCAGCTGAGTTAGTCTTCCCTGTGTGAGACACCCATGGG |
| | AAGCCATGCGCGGCCTCTGAGGAGAAAAGTCTCCTTATTGCCTTCATGTCTTTACGCCCGAGAGCAG |
| | AACCCCTCAGCGGCATTCCACAGGTTGCTCAGGCATATAACACTCCCTTGAAGCAGTGGAGTATAAT |
| | CAAACATCTTGGCTCCTCCTGAAACCCACTCCCACCCGTTTCAGTCCCGATAAGTTAAAGATTTGTT |
| | TTGTTTTGTTTTTGTTTGAGACGGAGTCTCGCTCTGTCGCCCAGGCTGGAGTGCGGTGGCTCGATCT |
| | CGGCTCACTGCAAGCTCCGCCTCCCGGGTTCACGCCATTCTCCCGCCTCAGCCTCCCGAGTAGCTGG |
| | GACGACAGGCGCCCGCCACCACGCCCGGCTAATTTTTTGTATTTTTAGTAGAGACGGGGTTTCACCA |
| | CGTTGGCCAGAGTGGTCTCGAACTCCTGACCTCAAGCGATCCACCCACCTCAGCCTCCCAAAGTGCT |
| | GGGATTACAGGCGTGAGCCACCGCGCCCGGCCAGTTAAAGATCTTAAGTAGTTTGACACTCCTCTTT |
| | GCTCAAGGAAATTCACAGAAACCGCCACTGCTATACATCTTACAGAATGACTCTCCAGTTCTCCTTC |
| | ACTGATTAATCCTTTCCCTCATCCCTTCCTCCTCCTCCCATCTGCCCTAAGAACAAAGAGCTTGTAA |
| | ACCAATAAATTGGGCGGAGCCTGAGAACTCTGGGCCGTGAGCAAGCCTCCGACGCTCCGGTCCCCTG |
| | GACCCGCCTTTTAAACGCTTATTCTGTCTCTTTCTAACTCCTTTGTCTCCGCCGGACTCGGGGTAAC |
| | CGCTAGGCGTTATGGGGCTGTTTTCCCCAACATAGGCAACAGAGCAGGACAGTGTCTCTAAAAAAAC |
| | AAAACCAAAACTATATTTTGTACTATTCTGATAAAAATGACTTAGTTACAAACAAAGAACAAATCAA |
| | CAGATAGTCATGCTGTGGAGATCAGGAATATTCCTTCCCAGGGTAAATGAAAGACCAATTCCCTAAC |
| | GTCATGTGGATATACGCTTGTGGCTTAAGATAAAATTACCCGTGACAGCATCAAATACCAGGGATAA |
| | AACTCAGTCTTCAACACGCATATGTATCTCCTGGGGTTGAATCCTCTGGAGGTCTTGTTAAAAATGC |
| | AGATTCTGGTCAAGAGTTCGAGACCAGCCTGGCCAATATGGTGAAACCCTGTCTCTACTAAAAACAC |
| | AAAAATTAGCTGGGTGTGGTGGTGGACGCCTGTAGTCCCAGCTACTCAGGAGACTGAGGCAGGAGAA |
| | TTGCTTGAACCCGGGAGGTGGCAGTTTAGTGAGCTGAGATCGGGCCACTGCACTCCAGCCTGGGAGA |
| | CAGAGTGAGACTCTGTCAAAAAAAAAAAAAAAAAAAAATGCATATTCTGATTCAATAGGTCTGGGGC |
| | AGAGGTGTTTTTTTTGTTTGTTTGTTTTTTGTTTTTTGGTTTTTTTTTTGGTTTTTTTTTTTTGACA |
| | GAGTCTAGCTCTTTCACCTAGGCTGGAGTGCATGACACCATCCCAGCTCACTGCAACCTCCGCTTCT |
| | TGGGTTCAAGCGATTCTCCTGCCTCAGCCTCCTGAATAGCTGGGATTACAGGCGTGCACCACCACAC |
| | CCAGCTAAGTTTTGTATTTGTAGTAGAGATGGGGTTTCACCGTGTTGGCCAGGTAAGTTTTGTATTT |
| | GTATTTGGTCTTGAACTCCTGACCTCAGGTGATCCGCCCGCCTCGGCCTCCCAAAGTTCTGGGATTA |
| | CAGGCGTGAGCCACTGCACCCGGCCTGTTCTGCATTTCTAACAAGTTCCCAGGGGATGCTACTGCTG |
| | CTGGTCTTCAACCACACTTTGTGGAGCAAGGCTCTCAAAGACCTTGATGTATGTAGGAGAGAAAGCT |
| | GGGGTAGAGAGTGATGAGGGGAGAACGGGTGCGTGGGGAGATGCTCCCCTGTGCATCCTGGTCCCAT |
| | GTGAGGCTCCAACAATGCTCACCTACATCACAGGGAGAGCACCTAGCAGGAAATGAGTTCTGCTTTA |
| | GCATCCAGGCACAGGAGATTAGAGGCACAGGCAGGCAGTAGATTCTACTTCATTATTTGTGCAGCTG |
| | GACACAGAGCTTCCTTTCTTTTCCTTGATACTGTTTTATTCCATCTAAGTATGTAGGAGTAAGAGGG |
| | CTGTGTTACACTGTTTTCCCCACCTTTAATGCATCTGATCAACCTAGGAGCCCCCTAAGACCCTATA |
| | TTATCTCACTTTATCATCACAGCAAACCTGGGAGAAGGATATGGTTCCTGTTTTACAGATGAGGAAA |
| | CTAAGTCTCAGGGAGGTGAAACTACTGCCCAAGGATAGCCAAACAAAATACACGTCAGAAGTGGGAT |
| | GTGAAACGAAGCCTGTATGTCACCAGAGTCACCTATCCTCTCCCCCTCCAACCACCTAACCACACCA |
| | GGGAGTTGGCAGGAGATTCCTAGCCCACCCCTTACATTAAAATCCCTTTTAGGCGGGTGCCACTATC |
| | CAGTCCTTCTCAATTGCACCTAGTGAGACCACGAAAGATCTTCTACCTGGCTCCTGGTAGATGAGAT |
| | CTGGCTATACAGGTACTTGGGTGCAAACCTGCCCCTCTGCCCCTGGAGCTATCACCTCCAGATCCTG |
| | CTACTTGTACCTTTGCAGCCCCAGGTAGCCAGTGGCAAGGGCCAGGGGTGGCAGCAGGGCTGGGAGT |
| | GGAGAAGAGTGTGAGAAAGTGCTGCGGGGCTCAGGAGACACAGCAGGGAACCAAGGGGTCCTAAGGG |
| | TTGCAATAGAGGACAGGGGCAGGGAGTGCAGAGTGGTGGGAAGGGGGATGGGAGCTGGGTGCAGGAC |
| | ACATAAGAGATGGAGCATCCCGGCCACACACGGTGGCTCACACCTGTTATCCCAGCACTTTGGGAGG |
| | CCGAGGTGGGTGGATCACGAGGTCAGGAGATCGAGACCATCCTGGCTAACACGGTGAAACCCCGTCT |
| | CTACTAAAAACACAAAAAATTAGCCAGGCGAGGTGGTGTGCACCTGTAGTCCCAGCTTCTTGGGAGG |
| | CTGAGGCAGGAGAATGGCGTGAACCCAGGAGGTGGAGCTTGCAGTCAGCTGAGATCCCGCCACTGCA |
| | TTCCAGCCTGGGTGACAGAGTGAGACTCGTCTCAAAAAAAAAAAGAATAAAAGAAAAAAGAGGTGGA |
| | GCATCCTGCAGCCCTGGCCCCTAAAAGATTGGTGGGAGAGTGCCAGCTGCTCCACCCTAGTCACTTT |
| | GGGAACTGGTCTTTCAGTTCACGGCCTGCCATGTCCTCTCCTGCAAATCCTGGCACTGTTGAGGAGG |
| | TCCTTTCAGCCCTGGTTTGTCCACTCTAACCTTGAATATATTATACACACACTTTATGAGAGCTGAC |
| | GAGGGACCAGGTGCTGTTCTAGGCTCTGAGGTGCAGCTGTGGACATTTGGGTACAAAGTTCTTCTGG |
| | CAGGGTACTTACCTCCTGCTGGGGGTGGGGGAACCTGAACAGCCAACACATAAGTAAAGCAAGATCA |
| | TCTCGGTGTTGAGTGCCTTGAAGACAATAATTTAAACGGGTGGGAGGATAGAGTGTGTGAAGTGAAA |
| | AAGTTTGCTTTAGTCAGGGTAGTCAGGGAAAGCCTTTGGGAGCAGGTGATATTGAAAGGAAATCTGA |
| | CTGAGAAGGCAAATTCCATGCACAAATTAAAAGGCCAGGAGGCTAGTTGGGCTGTTGCGTGGGAGGA |
| | GCAGCTAGAATGCCGGAGTGACTGGGGGGATGGGAGCCAGGGGATAGGGAGGCAGATGGAATGGGAA |
| | AGGCGTGGGCAGGAAGAACTTGGTCATGAAGACCTTGCAGGTGAACCCACTGGGGCCTTAAGCCTGG |
| | AGGAACTTGACAGAATTTGCCTACTGTGTGGGGAACGGCTTGGAGGGGGTGTGGGCTTCAGGAGGCT |
| | GAGATGTCCTGTTTCTTGTGCCCCCTCCTTTCTTCCCAACACCCGAGAAACCTGGATGGGTGTGGGG |
| | ACCAGAGACCTGGAGGTGGCCAGATTGGGCTTTGGCGGGACGCTTAGCAGCCCTCGGGACCTGTTCA |
| | GACTGCGGCCTCCCACCTTCGGGAAGCATCGGCGCTGCCCATCTGCCCCTGCCTGGCGTCCAGGGAG |
| | TCCCGGCTGTGCAGCGCTTCCCTTGAAATGTCTCTCTGTCCTCCCATCCAGTGCCTGGGACCCGGCA |
| | GCGCCGTCGAGGCAGGGGGCTGCGAGGCGGGACCCAGTTGCACGTGGGCCCTGTGGGGTCACTCCCT |
| | TTCGGGGGTCCTCTAGCTCTTCACCCTGCGCGCGTGGGGCAGACCAGATGCCTCGAGGAGCTCCAGG |
| | ACCAGTGCCTATGGGGTAGTCCCTGCCGGCGGTGGGCCCCAGTCCCAGACTGCGGCGCGCTATTTCT |
| | TTCTGGGGTTCGTGTGAGCGTGGGCTGCCAGAATGGTGCCCACAAGCTGCTTTTGGGTGATTCAAAT |
| | CATTTATACAGATAGTGCCCCTGCAAAAAACATTTGCGCAGGGCCCCGCTTACGCCAGAGGATTGCG |
| | GGCCACTTCTGGGCATCGCTCCTCGTGGGGATGGGAGCATCTCCCTGGAGAGCCCTTTGCAAAGGCC |
| | AAGCGCCGGCCAAAGGCACACCGCTGGACGCGTTTCCTTCCTTCTGGAGAGATGACCAGGAATGCAG |
| | GATCCAAAGGGGGTCTTGGAGGGAGGGCGGGAAGGGCATCTCCGGATCTGGGCAGACCCAGGGCTGC |
| | CGGCTCCCCGAGGAGAATACGGGCTGGGGGCGAGGAGCCGGAGGGCAGGTCAGGCAGTGCATCAACC |
| | CTTGGCTCCTCCACCGCAGCCCCAGCCCGCAGGCTATCGCTCAGGCTTCTCTCTCCGGGTTATGTAA |
| | CCCCGGGACGGGACGTGGCAGCCGGGTGAGTGAGCGAAGGAGTAGGGGAGGGAAGGGAAAGGAGAGG |
| | AGGGGCAGGGCCGGGCTTGGTGATGGTGGTGGTGGGAAGCGCCGCCGTGCCGCCTCTTCTTGGGCCC |
| | CTTGGGTTGTCTTTCTGGAGGATTCCGGGACCAGCCCTCTCCCCAGGCTCCGGGTCGCCCCCTAGCC |
| | CCCCGCCGCCTCATTTTCCCTTCACTCTTTTCCCCCTTCTGTCCCACCCGCCCTGCCAGGGGGCCTC |
| | TGGCTCTGGATAGCTTTTCCTCTCCGGTTGTAGTTTCCTTCCCAAAGTTCTCAGCTTTGCTACCTCG |
| | CCCAAGTCATTAGCCGCTCTGAGCCTCAGTTTATCAGTTTGTAAAATGAAGTTTGATTGAGCGGCCA |
| | CGTGTAAAACTCCTGGCATAGTGCATGGTACAAAGTAGATGTCTGCTGCAGGCTAAGGGCCTCGAGG |
| | GGCTAAGTGAAATGTTGTGTGCCAGGCTGGGTGTCAGAGCCCCGGGAGCCGCAGCCACGAATGGTTG |
| | GCTCCCGGGTGGTAAAAGAATTTATCAACAACAGTATAGGTTTGAAAAGTTTTATTAGATGGAAAGA |
| | ACTCCACAGCAGAGCGCAGCGGGATGCTTCGGCAAGAGAGGCCTGAGCTCACTTGCAGGGAACTGAA |
| | GGGTAATTTTGACCACATTAGTTTTGTAGGTCATAGTAAATGATTACATTTGTAGACATTTTGGCAC |
| | CTTGATGACAGCAAAGGTTGCACAATGGGTTCCAACATGCGTGCATTCCGGAGATGTATAGAAATTC |
| | TAGGGAAAGAAGCCTGGTACCAGATGTGGCTTTAGATAATAGGAAAGTACCATTCTGAGTTCTTCAG |
| | ATAAGGTGCTTTGCCTCCTGATGGTCTGCTTGATGGCCACCAGGTGATCCTTGCTCTCCTCATTTTC |
| | CCCCTGATAAATATTTTGGGCAAATCTTTGACCCTTTGTATTTCTCCATGCTCATGTCTACTTGTCT |
| | GTTAGGATCCCAAGAAAGGGAAAATGGCACAGTGAAGAGGGGTGTCCAGTCTATCTGGCTACTTCCT |
| | GCTGAAAAGGGGCATTGAAAGGATTCCTTTCTTGCTTTCTGTCATGAAGGGAATGAAGGGTCATGAT |
| | AAACTTGTTCATGGAGGGAAGACCAGATTCCATCAAGAGGCCCCATGAAAATAGAAGTTGCTGTTGC |
| | AGGCTGGTATTGGGATTGCATAGTCATCTGTAGGTGGAATCATTGTAAGCTGGAAGATATAAGCATT |
| | AAAAGGCAGGAATTACCGGCATGCACCTCCATGCCCACAGATTTTTGTGTTTTTAGTAGAGACAGGT |
| | TCTCACCATGTTGGCCAGGCTGGTCTCCAACTCCTGACCTCAGGTGATCCGCCCGCCTCGCCTTGGT |
| | CTCCCAAAGTACTAGGATTACAGGTGTGAGCAACCACACCTGGCCCCTGGGGTCTCAATTTGTGTAT |
| | TTATGCATGGCCTCCACCAGTCTAGCTTGGAAAAGGGCAGGGCTTTCAGATAGTTTCATACATACAA |
| | AATTATTATTTCTTTTTATTTTATTTTATTTGAGATGGAATTTCGCTCTTGTTGCCCAGGCTGGAGT |
| | GCAGTGGCGCAATCTCAGCTCACCACAACTTCCGCCTCCAAGGTTCAAACGATTCTCCTGCCTCAGC |
| | CTCTGGAGTAACTGGGATTACAGGCATGCACCACCATGCCCAGCTCATTTTGTATTTTTAGTAGAGA |
| | TGGGGTTTCTCCGTGTTGGCTAGGCTGGTCTCAAACCTCAGGTGACCCGCTCGCCTCAGCCTCCCAA |
| | AGTGCTGGGATTACAGGTGTGAGCCACCGCGCCCAGCTATTATTTCTTATAATTTAGAAAAATTAAC |
| | AGGTTTTATTATATATTTTTCATTCCCTCCAACAGAGAAGTTACCATATGATCCTGTCTGCCCTTAC |
| | CTCTGTTTGGGCCAGAATTGGTGGCCTGGTATTGCCAATAGGTTCTATGTTGGGGACAGCTTCTGCC |
| | CAGCTCTGTTATTAGGACTGGGAGCATGAGCTTCATCTGCCCATGCTGAAGATCACACGTGTGATTT |
| | TTTGTGTGTGGGAACAGCAGGTAGTTAATACCACAAATACATCTTGCCAGGTTAAATCAAAGGCAAC |
| | AGTTAAAGTCTGAAATTCTTGAATGAACTTAGAGGGATCCTGACTAAATGAACCCAACTTGGATTGA |
| | ATTTGCAAAAGATCAGACATGATCAGAAAAGGGACATGAACTTGGCTTGTTCCCAAATCTTCATTAG |
| | CCACCTTAGGGAGAGGCAAAATATTTTGGGGATTTTTCTGAGGACTCTGTACTAGTAGCATATGTGA |
| | CTCCCCTGAGAGTATGTGAAGGGGAGAAAGTATTTGGGTATGTGGGTGGGAGATTGACTAGGGAATG |
| | GAGCAGATGGAGAGGGTGTAGGTGAAGAGTGAGCAGGTTGAGGAGGATGTAATAGGCAAAAGGAAGG |
| | ATCATCTAAGACATCAGAACCGGGAAGGGAGGACGTTCCTTGGAAGCATACATGACAATTTGTATGT |
| | AATTTTGGGTTTGGATTTGGGGATAAAGCAAAAAAGACCTGAACATATGGGACTTCTGAATCCTTTC |
| | CAAGGTTCCGGCAAAAAATCAGTTAAGTTGTAAAGTAGCATTGCAATCCCAAGTTTCATTAATTGGC |
| | CAAATTGATTGATTAGGGAGCTTGTATTGAACCCAAGCAATATTAGAAAAAAGGATATGCTTTTTAA |
| | ACTCTTATTTATTTTTTATTTGTATTTTTTGAGACAGAGTCTTGCTGTGTCGCCCAGGCTGGAGTGC |
| | TGTGGCGCCATCTTGGCCCACTGCAACCTCCGCCCCCGGGGTACAAGTGATTCTCCTGCCTCAGCCT |
| | CCCTAGTAGCTGGGATTATATGTGCCCGCCACATATAATTAGCCCCCTGGCTGATTTTTTTTTTTTT |
| | TTTGTATTTTTAGTAGAGACAGGGTTTCGCCATGTTGGCCAGGCTGATCTCGAACTCCTGACCTCAG |
| | GTGATCCACTCGCCTCGGCCTCCCAAAGTGCTAGGATTACAGGTGTGAGTCACTGTGCCCGGCCAAG |
| | TTTTGCATTTTTAGTAGACTCCCGGTCTTTAACTCCGGACCTCAGGTGATCTGCCTGCCTTGGCCTC |
| | CCAAAGTGCTGGGGTTACAGGCATAAGCCATTGTGCTCAGCCTTATATGCTTATTTTTAAGAGTTTG |
| | TGGGTCAAAATGAGACCAATGGGACCATTTTTAAGGAGGCAATCCAAGGGCGAGTTGGATGGAACTG |
| | AATTAATTGAACCGAAGTTGGGTTTAGACAAGGAACTACAAGATCCCTGAGGCATCCCTGTGTAGAA |
| | TTGAGATCCACCGCTTCCAGGACAAGGCTTATGGAGTGTTAAAATGAAAGTGCCCTGCCACTCTGAC |
| | AGGCAATAGCTCTTTTGTCTTGGCCTTGGGGTAATACCGGGGGATGGCGCTTGGCCAGAAACTGTCA |
| | GTTGCCAACGAGAACTCAAGCTGGTTCACTGGCAGTCCGAAAACAGAAAAGAGCCCTGGCCAGTCCC |
| | TCACCCCTAAGGGCAAGGACAGCCAGGTATCCCTTCTCTAGGGCTTCAGGATCCCACAGAAGAGCTG |
| | CCTCCACCGGGACCGGCAGTTCCCCAAAGAGTAAAGAACCAGACCGTGGAAGGAAGCAGAGAGAAAA |
| | AGGAAGAGGGAAATCCCAGTGAAGTCCCCGTATGGGCCACCAAGATGCCAGGCGAGGTGTCAGAGCT |
| | CCGGAACCGGGAAGTGGTTGGCTCCCGGGTGGTAAAAGAACTTATCAACAACCGTGTAGGTCTGAAA |
| | AGGAAAGTTTTATTAGACGGAAAGGACGAGGCAGCAGAGCGCAGTAGGCGCTTCAGCAAGAGAGGAC |
| | TGAGCTCCCTGCGGGGAACTGCAGGGTAATTTGGACCACATTAGTCACTTAGGTCATGGTAAATGGT |
| | TACATTTGTCGATATTTTGGTGCCTTGATGTCAGCAAAGTTTGCACAATGGGTCTTAACGTGCACTC |
| | ATTCCGGAAACGTACAGAAATTCTAGTTACTTATAAATTCTTGGGACGGAAGCTTGGTACCAGATGT |
| | GGCTTTAGACAATAGGGAAGTGTCATTCTGAATTGCTCAGATAAGGGGCTTTGCCTCCTGTTGGTCG |
| | ACTTGATGGCCACCAGGTGATCTCTGGTCTCTTCAGTGTGGCTTTGCAGACTATAAAGGCGCAGCGC |
| | GCCAACGAGGCGGGTTGGCCCCAGACGGCGGAGAGGAAGGGCAGAGTCGGCGGTCCTGAGACTTGGG |
| | GCGGCCCCTTGGAGGTCAGCCCCGCTCGCTCCTCCCGGCCCTCTCCTCCTCTCCGAGGTCCGAGGCG |
| | GGCAGCGGGCTGTGGGCGGGCAGGAGGCTGCGGAGGGGCGGGGGGCAGGAAGGGGCGGGGGGCTCGG |
| | CGCACTCGGCAGGAAGAGACCGACCCGCCACCCGCCGTAGCCCGCGCGCCCCTGGCACTCAATCCCC |
| | GCCATGTGGGGGCTCCTGCTCGCCCTGGCCGCCTTCGCGCCGGCCGTCGGCCCGGCTCTGGGGGCGC |
| | CCAGGAACTCGGTGCTGGGCCTCGCGCAGCCCGGGACCACCAAGGTCCCAGGCTCGACCCCGGCCCT |
| | GCATAGCAGCCCGGCACAGCCGCCGGCGGAGACAGCTAACGGTGAGTTCCCCGACCGACGGTCCGCT |
| | CCCCCGCAAGCCGACTGCCCGGCTCTCCTGCCCCGTGGGGCGATCCCTCCCTAACACGCGGGCACAC |
| | GCACACCCACGCACACTCACAGTCATGCACACTCACCCCGCACGCACACTCGCACTCACGCGCACAC |
| | ACGCGCGCGCACTCACACACATTCACACACGCGCACACTTGCACTCACACGCGCGCGCATTCACACG |
| | CATGCACACACACGCACACTCACACGCGCGTGCGCGCACACACAGTGCACGCGCGCGCACACTCACA |
| | CTCACAGTGCACACACACATATACACACTCACACTCCCTCAACTCCCTGCTGGGAGCAATGGCTGCT |
| | GACTCGGCAGCCCCAGTTCCCTGCCAGACCTAGTCAGCAGTCCCAGGACAGGCGCCAGTGGGATGCT |
| | GCCTCTTCCAAGCCCCAAACCTTCCCTTTTCACCAAAGACAAAACAGGCCAGAACTGGCAGGAGGGG |
| | AGACAGAGGGGCAGAAGCTCTCAAGGTGCAGAGCAAGACTGCGTAGGAGAGAGTTTGAAGGCGAGGG |
| | CTGGAGAGAAAGAACAAAAGGAAAGAAGGGAGAGCCCCTCGCTGAGGCTGCCGGGAGGATGGGGCAG |
| | AGCGGGAGAGGAAGGCAGCCCGACCTCCCAGCTTTCCAGATGTGGAATAGGAGAGGAGGAGCGCAAG |
| | CGGAGGGCACTCAGGGGCTTCTAGAGGAGGCAAGTGGAGGAGGGTCTTGAAGGGTGATGTCCCCGAG |
| | TCAGGGGAGTCTGGAGAGAGAGAGAGAGAGAGGGCTGCCAAGAAGGAAGCGGCGGGCAAAGGCACAG |
| | GGGCACCAGATGCGGAAATGGGCAGCCTGTTCTGGAGGCAGCTGTGGAGCTTCGATGGGTACCCCCA |
| | GCACCTGCCTGGGCAGAGCCTTGTGCTGAAGGGCCGGCGGGCAGGCCCAGCCCTGAAAGCCTCGACA |
| | CCCAGGCAGACATGGATTCCAGGACAGGCCATCTGAGCCCAGAGAGCAGACACAACAATGGAAGCGG |
| | CACAGGGGTTTTGGGGCATGATGCTGAGTCTGGAGCTAAGAAAGCCTCCTTGGAAAGGCATCTGGGC |
| | TGAGATGCAAAGGAAGAATGGGAATTAGGTGAAAAAATCAGAGGCGAGGGGTAGCATTACAGGGGAG |
| | GGGATAGCTAGTGCAGAGGCCCGGAGGTAAAGTGCCAGACTCAGCTCTTTGGAGCAACCGAACAGTT |
| | TCTAGAGGCTGGGTGCAGCTCTCCATTGGATTAGAGGTTCACAGGGGAGGCTGGCCAAGCATGTAGT |
| | TACATCAGGGAGGAGAAGGAGGAGCCAAGGAAGTGACTGGAGAGGCAGGTTGGGGTCAGATTGCAGG |
| | CCTTTGATGTCCTGTGAAGGCTGTTAGATCCTGGTGGTGTGGCCTGCTGTGGGCTCACATGTCTTCT |
| | TGGGCTGGCAGACCTTTCCATCCGGGGTTTCACCATTCTTCCTTTCCCCCATGCTGTGCCTCTCGGA |
| | CCCCAAGGGACCTCAGAACAGCATGTCCGGATTCGAGTCATCAAGAAGAAAAAGGTCATTATGAAGA |
| | AGCGGAAGAAGCTAACTCTAACTCGCCCCACCCCACTGGTGACTGCCGGGCCCCTTGTGACCCCCAC |
| | TCCAGCAGGGACCCTCGACCCCGCTGAGAAACAAGAAACAGGTACTTCCTCTCCAGGGGCCCAGCCC |
| | AGACTTGCAGCCCCTGGGGCACTTTACCAGCACAGCTCTTGGCCTCATGGGCACCGGCACGCCCCTT |
| | GCTTGCCTAGCGCAGGAGCAACCTTAGGCTCAGCTTCCCACCTGCCCTGGCTACCCTCCCTCTGGTC |
| | CTGTCTCACTGTTCTATCCCCGCCCCAGGCTGTCCTCCTTTGGGTCTGGAGTCCCTGCGAGTTTCAG |
| | ATAGCCGGCTTGAGGCATCCAGCAGCCAGTCCTTTGGTCTTGGACCACACCGAGGACGGCTCAACAT |
| | TCAGGTCAGTAATCCTGGCTCGGAGCCATGGTCTCAGGGTAGGGAAGGCAGCCCCTGGGAGCTTCTC |
| | TCCTGCCTCCTCTCTGTCCTGGCCTGCCCCACTCTGTCCAACTGGGCCTGACCACCATGTCCTGTGT |
| | CTGCAGTCAGGCCTGGAGGACGGCGATCTATATGATGGAGCCTGGTGTGCTGAGGAGCAGGACGCCG |
| | ATCCATGGTTTCAGGTGGACGCTGGGCACCCCACCCGCTTCTCGGGTGTTATCACACAGGGCAGGAA |
| | CTCTGTCTGGAGGTGAGGCAGACTAACCCTAGGTCAGGAGGTCACAGAAGGACTGGGGTGGGAGTCC |
| | TGGGGGCACCGATGATCTCTCTCCACCTCTCCTGCCAGGTATGACTGGGTCACATCATACAAGGTCC |
| | AGTTCAGCAATGACAGTCGGACCTGGTGGGGAAGTAGGAACCACAGCAGTGGGATGGACGCAGTGAG |
| | TGGTCCCACTGTGGCTGGGGCCTCCATGCTGGGAGTTGGGCACCCAGTCCAGGCTAGGCTGAGGCTC |
| | CTCTGAGGACAAGGAATAGACGCCAGCTTAGGCTTCCCAGGGGGGTGTGGCTTGTTGTCAAGAGGGT |
| | GGCACACGGCAGGCACCATTGGGAGCCAGCTGCTTTGGGACATGCCCACATCCTCCCCAGATAATGC |
| | CACCACAGGGTGGGTGCTGCTTCACGGTACAGCTTCCTCCTGGCGTGCCCCTTCTGGCCCGGGGCCT |
| | CTGGTCCACATCACTTCTTGCCTTCTCGTGGTTCTGACTTCCGCATCTCATGGACCTCTTTTTACAG |
| | CAGGCTACAATGTGGAGTCCTGGCCAGCTCTAGGATTGGCTTCCCCCGAGTCATGTGGCCAAACTGG |
| | TCTAATGAACTGTGTCCAATCCAGAGAGCAAGGCTGCCTAGGGCTGCCCATTGGCAGGGGCTGTGGG |
| | CCGGGGTCTGTGTTTGATGCACAGTGCAAGTCTCTAGCTGAGCCCACTAGGGTGGGGAGACAGTAAG |
| | CTTGGAGGCCTGAGCTCCTTCCCTGGGTCCTGGGCCAGGCTTCTGGGGTTTGAGCAGCCACAACAGA |
| | GAACTTGCTGCCCCCAGGTATTTCCTGCCAATTCAGACCCAGAAACTCCAGTGCTGAACCTCCTGCC |
| | GGAGCCCCAGGTGGCCCGCTTCATTCGCCTGCTGCCCCAGACCTGGCTCCAGGGAGGCGCGCCTTGC |
| | CTCCGGGCAGAGATCCTGGCCTGCCCAGTCTCAGGTGGGCAGTCAGGCCAGGGTTGGTTGGGCAGGG |
| | CTTGGATGCAGGGTGCATCCTTCACTGTGGACACACCCTTTACCATAAACTCAACCTCCACCAGACC |
| | CCAATGACCTATTCCTTGAGGCCCCTGCGTCGGGATCCTCTGACCCTCTAGACTTTCAGCATCACAA |
| | TTACAAGGCCATGAGGAAGGTCAGATATAACCCCTATGACCTGGGAAGGAGGGCCCACCCATCTCAG |
| | GTCCCCTTCCCACCTTCCCACCGGGGCACAACCTGCTGTGACTGCGCTTGTATGCCCCTGCTGCCTC |
| | CTGATGTCTCAGCCTTCTCTCCTGTGGACCCCTAAGCTCCATCCCACTTTCCCTTATTATGGCGCCC |
| | CCCCAGTCCTACCCCTTCCTCCCGGCTCTGCTGCCGCTCCCCTCCTGTACCATGATGGGATGCCCCC |
| | TCTGTGTGGGCCATCGCTGACTTTTTAAGTCTTTCCATGGCACATGTGATCTGCCCCTGGGTGTACC |
| | CCTCCCATGCCTCATGCCACGCTACACTCTGCCCACCAGCTGATGAAGCAGGTACAAGAGCAATGCC |
| | CCAACATCACCCGCATCTACAGCATTGGGAAGAGCTACCAGGGCCTGAAGCTGTATGTGATGGAAAT |
| | GTCGGACAAGCCTGGGGAGCATGAGCTGGGTACTGGCATGGGGAGTGGGGAGAGGTAGGCACAGGGC |
| | AGGGCCCCAGGCATGAACCCGCTGCAAGCCCCCATGTGTCCCCAGGGGAGCCTGAGGTGCGCTACGT |
| | GGCTGGCATGCATGGGAACGAGGCCCTGGGGCGGGAGTTGCTTCTGCTCCTGATGCAGTTCCTGTGC |
| | CATGAGTTCCTGCGAGGGAACCCACGGGTGACCCGGCTGCTCTCTGAGATGCGCATTCACCTGCTGC |
| | CCTCCATGAACCCTGATGGCTATGAGATCGCCTACCACCGGGTAGGCCACCCAGCATGAGGGCCACT |
| | CTGTCCTTCTGCCCTGGTGGCTGGACCTGCTCGACTTGAACAAGCCTCTTGCCCGGCAGGGTTCAGA |
| | GCTGGTGGGCTGGGCCGAGGGCCGCTGGAACAACCAGAGCATCGATCTTAACCATAATTTTGCTGAC |
| | CTCAACACACCACTGTGGGAAGCACAGGACGATGGGAAGGTGCCCCACATCGTCCCCAACCATCACC |
| | TGCCATTGCCCACTTACTACACCCTGCCCAATGCCACCGTGAGTATTTTGAGGGCGGCAGTGGAGGT |
| | CTGTGGGGGGCGGACCTTGTCTCTGTCTCCTGCCCCTCCTGACCTGCCCCATCCAGGTGGCTCCTGA |
| | AACGCGGGCAGTAATCAAGTGGATGAAGCGGATCCCCTTTGTGCTAAGTGCCAACCTCCACGGGGGT |
| | GAGCTCGTGGTGTCCTACCCATTCGACATGACTCGCACCCCGTGGGCTGCCCGCGAGCTCACGCCCA |
| | CACCAGATGATGCTGTGTTTCGCTGGCTCAGCACTGTCTATGCTGGCAGTAATCTGGCCATGCAGGA |
| | CACCAGCCGCCGACCCTGCCACAGCCAGGACTTCTCCGTGCACGGCAACATCATCAACGGGGCTGAC |
| | TGGCACACGGTCCCCGGGAGTATGTGCCTGAGGGTGGAGTTAGCCCTGGCCCCGTAACCCCCGCCCT |
| | GATAAGACAGCCTGCGGTTGCGTACAGTGCTGGCGTCTGTTCCCACTCTGAAGTGTCCCTCAGAGAA |
| | GGGAGGGTAGCGGGAGGATGGGACCGCATCCCGCCTGCTTAGGCAGCAGTGTCTGTGGTCCCCTTAG |
| | GCATGAATGACTTCAGCTACCTACACACCAACTGCTTTGAGGTCACTGTGGAGCTGTCCTGTGACAA |
| | GTTCCCTCACGAGAATGAATTGCCCCAGGAGTGGGAGAACAACAAAGACGCCCTCCTCACCTACCTG |
| | GAGCAGGTCGGATCTGCGTCCCGGCCCCCAGCCTGCCTGAATCACTCCTGCTGTCCATTTAGGCTAC |
| | AGCTCCTACCAGGGGTTCTTCTAAGGTCCAGCTGAGCATTCAGACTCACAAGATGCCATGGGCCATG |
| | CTTGGTATCAGATTGTCTTGGAAGCACACAGGACAGGAAGTGCAGTTTGCTGGCAGCGTGGCATCGT |
| | GTTAGAGCCGGTGGGAGGAGCCTCCATTGCAGTCTAGGTGGTGGTCCGTGGCGCTGCCCCAGAGCTA |
| | TCCTCAGGAGAGACTCACGTGAGGCAGGTGCAGGAGCTGTCCTGGCATAGAAGCTTCATGTTCCATG |
| | GAGCTCATAACCCTTGTAATAGCTCCATAAGCAGAGCTTCCAAAGGGTCTACCAAAGACAAGCCCAA |
| | TAACCTGGGAAAGCCCAAGGATAGATAAGCCTTCCTACCAGGTATTTATCATTTTCTTAGTCCAGAT |
| | GTGATTTGTCAATCAGGATTTCTTTTTTTTTTTTCTTCCAGAAGTAGTGTCACCTAGGAACACAGTA |
| | GACCTACCACTTTGCTCAGGTTTGCAGGGCAACAGAGCCAGCAAGTTAGCTAAACAGCACATTATCC |
| | TGCCGAAGGGGAAGGGCTCTGATAACCTCTTCCCACACAGGTGCGCATGGGCATTGCAGGAGTGGTG |
| | AGGGACAAGGACACGGAGCTTGGGATTGCTGACGCTGTCATTGCCGTGGATGGGATTAACCATGACG |
| | TGACCACGGGTGTGTTTGACCGGGAGGGCAAGGGAAGGGGCTGGAGGGCTGGAGGCTCGGGAAGAAG |
| | CAGAAGATCATTAATTGGGTCCTGATCGTGCCCTTCACTCTCCTCAGCGTGGGGCGGGGATTATTGG |
| | CGTCTGCTGACCCCAGGGGACTACATGGTGACTGCCAGTGCCGAGGGCTACCATTCAGTGACACGGA |
| | ACTGTCGGGTCACCTTTGAAGAGGGCCCCTTCCCCTGCAATTTCGTGCTCACCAAGACTCCCAAACA |
| | GAGGCTGCGCGAGCTGCTGGCAGCTGGGGCCAAGGTGCCCCCGGACCTTCGCAGGCGCCTGGAGCGG |
| | CTAAGGGGACAGAAGGATTGATACCTGCGGTTTAAGAGCCCTAGGGCAGGCTGGACCTGTCAAGACG |
| | GGAAGGGGAAGAGTAGAGAGGGAGGGACAAAGTGAGGAAAAGGTGCTCATTAAAGCTACCGGGCACC |
| | TTAGCTCATCTTCGTGTTGTCTCTGTGCCCCAGGTCCTCCCCCCGGGGGCGGGCCTCGGCCCAGCCC |
| | TCAGTTCCTATTCTGCACACTTGCACACTCTCATCAGTTGGCTTCTGGACACATTGTGTGAAAAGAG |
| | GATCCCACCTGGGCTCTTCTTGAACCAAGGGCCTGGCAGAGCAACTCATTTCTTCTGATCAGCTTCT |
| | GCTACAGGTACCATTACACTGCTGCCAGGCATTCTGTAAGCGCCTGCTCATTGCCAGGTGTGCAAGG |
| | AATCAGGATCAGCCGTGCCTGCACTCAAACTCCTGGGGCTCCTAGTCAAGGGAAAGGACAGTTCGGT |
| | ACATTGTGAGACATGCTAGGGTGGAGGCCAGGTGCCGTGAGAGTGCAGGGGAGCTGCACACGTGAAA |
| | TACAGCACTGCACATCAACAGGACTGGGGCAGTCAAGGATGCAATAGAAGTAGTGGCTCTAGAAGTT |
| | CAGGCGGGAGGTGGGCAGGGTGTGGAGTATGGACAGGGATGGCTCCAAGGAGGAGGGTCAGCCAAAG |
| | GTGGGTCAGCTGAGAACATTTGAATTTGCTTCAGCCATTCTCAGAGTATTGATAACTGATAGGCTTT |
| | GCTGAGTTTCTATCAGACTGAAGGGGAAGTTGTGTATCAGTCTGTGTCTTGCCAGGTAAACAACCCA |
| | TTCTAGGCACTTAAAGTGGAGGGAAATTTAATGCTGGAAATTGGATAGGAAGGTGTTGGAAGAGCTG |
| | GATGAGGCCGGGTGTGGTGGCTCACACCTGTAATCCCAGCACTTTGGGAGGCTGAGGTGGGAGGATT |
| | GCTTGAGCCCAGGAGTTTGAGACCAGCCTGGATAACATAGCCAAACCCCGCCTCTACAAAAATAAGA |
| | AATAAGAAACATAGCCAGCTGTAGTGGCGCATCGCTAAGGGAGGCAGAGGCAGGAGGATCACTGGAG |
| | CCTGGGAGGTGGAGGCTGCAGAGGCAGCAGTGAGCCATGATGGCGCCACTATACTCCAACCTGGATG |
| | GTCATAACAAAATAAACAAAAAA |

| | | | |
|---|---|---|---|
| ORF Start: ATG at 1 | | ORF Stop: TGA at 2203 | |
| | SEQ ID NO: 154 | 734 aa | MW at 81666.8 kD |

| | |
|---|---|
| NOV22c, | MWGLLLALAAFAPAVGPALGAPRNSVLGLAQPGTTKVPGSTPALHSSPAQPPAETANGTSEQHVRIR |
| CG54007-01 | |
| Protein Sequence | VIKKKKVIMKKRKKLTLTRPTPLVTAGPLVTPTPAGTLDPAEKQETGCPPLGLESLRVSDSRLEASS |
| | SQSFGLGPHRGRLNIQSGLEDGDLYDGAWCAEEQDADPWFQVDAGHPTRFSGVITQGRNSVWRYDWV |
| | TSYKVQFSNDSRTWWGSRNHSSGMDAVFPANSDPETPVLNLLPEPQVARFIRLLPQTWLQGGAPCLR |
| | AEILACPVSDPNDLFLEAPASGSSDPLDFQHHNYKAMRKLMKQVQEQCPNITRIYSIGKSYQGLKLY |
| | VMEMSDKPGEHELGEPEVRYVAGMHGNEALGRELLLLLMQFLCHEFLRGNPRVTRLLSEMRIHLLPS |
| | MNPDGYEIAYHRGSELVGWAEGRWNNQSIDLNHNFADLNTPLWEAQDDGKVPHIVPNHHLPLPTYYT |
| | LPNATVAPETRAVIKWMKRIPFVLSANLHGGELVVSYPFDMTRTPWAARELTPTPDDAVFRWLSTVY |
| | AGSNLAMQDTSRRPCHSQDFSVHGNIINGADWHTVPGSMNDFSYLHTNCFEVTVELSCDKFPHENEL |
| | PQEWENNKDALLTYLEQVRMGIAGVVRDKDTELGIADAVIAVDGINHDVTTAWGGDYWRLLTPGDYM |
| | VTASAEGYHSVTRNCRVTFEEGPFPCNFVLTKTPKQRLRELLAAGAKVPPDLRRRLERLRGQKD |

| | |
|---|---|
| SEQ ID NO: 155 | 2202 bp |

| | |
|---|---|
| NOV22d, | ATGTGGGGGCTCCTGCTCGCCCTGGCCGCCTTCGCGCCGGCCGTCGGCCCGGCTCTGGGGGCGCCCA |
| CG54007-02 | |
| DNA Sequence | GGAACTCGGTGCTGGGCCTCGCGCAGCCCGGGACCACCAAGGTCCCAGGCTCGACCCCGGCCCTGCA |
| | TAGCAGCCCGGCACAGCCGCCGGCGGAGACAGCTAACGGGACCTCAGAACAGCATGTCCGGATTCGA |
| | GTCATCAAGAAGAAAAAGGTCATTATGAAGAAGCGGAAGAAGCTAACTCTAACTCGCCCCACCCCAC |
| | TGGTGACTGCCGGGCCCCTTGTGACCCCCACTCCAGCAGGGACCCTCGACCCCGCTGAGAAACAAGA |
| | AACAGGCTGTCCTCCTTTGGGTCTGGAGTCCCTGCGAGTTTCAGATAGCCGGCTTGAGGCATCCAGC |
| | AGCCAGTCCTTTGGTCTTGGACCACACCGAGGACGGCTCAACATTCAGTCAGGCCTGGAGGACGGCG |
| | ATCTATATGATGGAGCCTGGTGTGCTGAGGAGCAGGACGCCGATCCATGGTTTCAGGTGGACGCTGG |
| | GCACCCCACCCGCTTCTCGGGTGTTATCACACAGGGCAGGAACTCTGTCTGGAGGTATGACTGGGTC |
| | ACATCATACAAGGTCCAGTTCAGCAATGACAGTCGGACCTGGTGGGGAAGTAGGAACCACAGCAGTG |
| | GGATGGACGCAGTATTTCCTGCCAATTCAGACCCAGAAACTCCAGTGCTGAACCTCCTGCCGGAGCC |
| | CCAGGTGGCCCGCTTCATTCGCCTGCTGCCCCAGACCTGGCTCCAGGGAGGCGCGCCTTGCCTCCGG |
| | GCAGAGATCCTGGCCTGCCCAGTCTCAGACCCCAATGACCTATTCCTTGAGGCCCCTGCGTCGGGAT |
| | CCTCTGACCCTCTAGACTTTCAGCATCACAATTACAAGGCCATGAGGAAGCTGATGAAGCAGGTACA |
| | AGAGCAATGCCCCAACATCACCCGCATCTACAGCATTGGGAAGAGCTACCAGGGCCTGAAGCTGTAT |
| | GTGATGGAAATGTCGGACAAGCCTGGGGAGCATGAGCTGGGGGAGCCTGAGGTGCGCTACGTGGCTG |
| | GCATGCATGGGAACGAGGCCCTGGGGCGGGAGTTGCTTCTGCTCCTGATGCAGTTCCTGTGCCATGA |
| | GTTCCTGCGAGGGAACCCACGGGTGACCCGGCTGCTCTCTGAGATGCGCATTCACCTGCTGCCCTCC |
| | ATGAACCCTGATGGCTATGAGATCGCCTACCACCGGGGTTCAGAGCTGGTGGGCTGGGCCGAGGGCC |
| | GCTGGAACAACCAGAGCATCGATCTTAACCATAATTTTGCTGACCTCAACACACCACTGTGGGAAGC |
| | ACAGGACGATGGGAAGGTGCCCCACATCGTCCCCAACCATCACCTGCCATTGCCCACTTACTACACC |
| | CTGCCCAATGCCACCGTGGCTCCTGAAACGCGGGCAGTAATCAAGTGGATGAAGCGGATCCCCTTTG |
| | TGCTAAGTGCCAACCTCCACGGGGGTGAGCTCGTGGTGTCCTACCCATTCGACATGACTCGCACCCC |
| | GTGGGCTGCCCGCGAGCTCACGCCCACACCAGATGATGCTGTGTTTCGCTGGCTCAGCACTGTCTAT |
| | GCTGGCAGTAATCTGGCCATGCAGGACACCAGCCGCCGACCCTGCCACAGCCAGGACTTCTCCGTGC |
| | ACGGCAACATCATCAACGGGGCTGACTGGCACACGGTCCCCGGGAGCATGAATGACTTCAGCTACCT |
| | ACACACCAACTGCTTTGAGGTCACTGTGGAGCTGTCCTGTGACAAGTTCCCTCACGAGAATGAATTG |
| | CCCCAGGAGTGGGAGAACAACAAAGACGCCCTCCTCACCTACCTGGAGCAGGTGCGCATGGGCATTG |
| | CAGGAGTGGTGAGGGACAAGGACACGGAGCTTGGGATTGCTGACGCTGTCATTGCCGTGGATGGGAT |
| | TAACCATGACGTGACCACGGCGTGGGGCGGGGATTATTGGCGTCTGCTGACCCCAGGGGACTACATG |
| | GTGACTGCCAGTGCCGAGGGCTACCATTCAGTGACACGGAACTGTCGGGTCACCTTTGAAGAGGGCC |
| | CCTTCCCCTGCAATTTCGTGCTCACCAAGACTCCCAAACAGAGGCTGCGCGAGCTGCTGGCAGCTGG |
| | GGCCAAGGTGCCCCCGGACCTTCGCAGGCGCCTGGAGCGGCTAAGGGGACAGAAGGAT |

| | | |
|---|---|---|
| ORF Start: ATG at 1 | | ORF Stop: end of sequence |
| SEQ ID NO: 156 | 734 aa | MW at 81666.8 kD |

| | |
|---|---|
| NOV22d, | MWGLLLALAAFAPAVGPALGAPRNSVLGLAQPGTTKVPGSTPALHSSPAQPPAETANGTSEQHVRIR |
| CG54007-02 | |
| Protein Sequence | VIKKKKVIMKKRKKLTLTRPTPLVTAGPLVTPTPAGTLDPAEKQETGCPPLGLESLRVSDSRLEASS |
| | SQSFGLGPHRGRLNIQSGLEDGDLYDGAWCAEEQDADPWFQVDAGHPTRFSGVITQGRNSVWRYDWV |
| | TSYKVQFSNDSRTWWGSRNHSSGMDAVFPANSDPETPVLNLLPEPQVARFIRLLPQTWLQGGAPCLR |
| | AEILACPVSDPNDLFLEAPASGSSDPLDFQHHNYKAMRKLMKQVQEQCPNITRIYSIGKSYQGLKLY |
| | VMEMSDKPGEHELGEPEVRYVAGMHGNEALGRELLLLLMQFLCHEFLRGNPRVTRLLSEMRIHLLPS |
| | MNPDGYEIAYHRGSELVGWAEGRWNNQSIDLNHNFADLNTPLWEAQDDGKVPHIVPNHHLPLPTYYT |
| | LPNATVAPETRAVIKWMKRIPFVLSANLHGGELVVSYPFDMTRTPWAARELTPTPDDAVFRWLSTVY |
| | AGSNLAMQDTSRRPCHSQDFSVHGNIINGADWHTVPGSMNDFSYLHTNCFEVTVELSCDKFPHENEL |
| | PQEWENNKDALLTYLEQVRMGIAGVVRDKDTELGIADAVIAVDGINHDVTTAWGGDYWRLLTPGDYM |
| | VTASAEGYHSVTRNCRVTFEEGPFPCNFVLTKTPKQRLRELLAAGAKVPPDLRRRLERLRGQKD |

| | |
|---|---|
| SEQ ID NO: 157 | 2142 bp |

| | |
|---|---|
| NOV22e, | GCGCCCAGGAACTCGGTGCTGGGCCTCGCGCAGCCCGGGACCACCAAGGTCCCAGGCTCGACCCCGG |
| CG54007-03 | |
| DNA Sequence | CCCTGCATAGCAGCCCGGCACAGCCGCCGGCGGAGACAGCTAACGGGACCTCAGAACAGCATGTCCG |
| | GATTCGAGTCATCAAGAAGAAAAAGGTCATTATGAAGAAGCGGAAGAAGCTAACTCTAACTCGCCCC |
| | ACCCCACTGGTGACTGCCGGGCCCCTTGTGACCCCCACTCCAGCAGGGACCCTCGACCCCGCTGAGA |
| | AACAAGAAACAGGCTGTCCTCCTTTGGGTCTGGAGTCCCTGCGAGTTTCAGATAGCCGGCTTGAGGC |
| | ATCCAGCAGCCAGTCCTTTGGTCTTGGACCACACCGAGGACGGCTCAACATTCAGTCAGGCCTGGAG |
| | GACGGCGATCTATATGATGGAGCCTGGTGTGCTGAGGAGCAGGACGCCGATCCATGGTTTCAGGTGG |
| | ACGCTGGGCACCCCACCCGCTTCTCGGGTGTTATCACACAGGGCAGGAACTCTGTCTGGAGGTATGA |
| | CTGGGTCACATCATACAAGGTCCAGTTCAGCAATGACAGTCGGACCTGGTGGGGAAGTAGGAACCAC |
| | AGCAGTGGGATGGACGCAGTATTTCCTGCCAATTCAGACCCAGAAACTCCAGTGCTGAACCTCCTGC |
| | CGGAGCCCCAGGTGGCCCGCTTCATTCGCCTGCTGCCCCAGACCTGGCTCCAGGGAGGCGCGCCTTG |
| | CCTCCGGGCAGAGATCCTGGCCTGCCCAGTCTCAGACCCCAATGACCTATTCCTTGAGGCCCCTGCG |
| | TCGGGATCCTCTGACCCTCTAGACTTTCAGCATCACAATTACAAGGCCATGAGGAAGCTGATGAAGC |
| | AGGTACAAGAGCAATGCCCCAACATCACCCGCATCTACAGCATTGGGAAGAGCTACCAGGGCCTGAA |
| | GCTGTATGTGATGGAAATGTCGGACAAGCCTGGGGAGCATGAGCTGGGGGAGCCTGAGGTGCGCTAC |
| | GTGGCTGGCATGCATGGGAACGAGGCCCTGGGGCGGGAGTTGCTTCTGCTCCTGATGCAGTTCCTGT |
| | GCCATGAGTTCCTGCGAGGGAACCCACGGGTGACCCGGCTGCTCTCTGAGATGCGCATTCACCTGCT |
| | GCCCTCCATGAACCCTGATGGCTATGAGATCGCCTACCACCGGGGTTCAGAGCTGGTGGGCTGGGCC |
| | GAGGGCCGCTGGAACAACCAGAGCATCGATCTTAACCATAATTTTGCTGACCTCAACACACCACTGT |
| | GGGAAGCACAGGACGATGGGAAGGTGCCCCACATCGTCCCCAACCATCACCTGCCATTGCCCACTTA |
| | CTACACCCTGCCCAATGCCACCGTGGCTCCTGAAACGCGGGCAGTAATCAAGTGGATGAAGCGGATC |
| | CCCTTTGTGCTAAGTGCCAACCTCCACGGGGGTGAGCTCGTGGTGTCCTACCCATTCGACATGACTC |
| | GCACCCCGTGGGCTGCCCGCGAGCTCACGCCCACACCAGATGATGCTGTGTTTCGCTGGCTCAGCAC |
| | TGTCTATGCTGGCAGTAATCTGGCCATGCAGGACACCAGCCGCCGACCCTGCCACAGCCAGGACTTC |
| | TCCGTGCACGGCAACATCATCAACGGGGCTGACTGGCACACGGTCCCCGGGAGCATGAATGACTTCA |
| | GCTACCTACACACCAACTGCTTTGAGGTCACTGTGGAGCTGTCCTGTGACAAGTTCCCTCACGAGAA |
| | TGAATTGCCCCAGGAGTGGGAGAACAACAAAGACGCCCTCCTCACCTACCTGGAGCAGGTGCGCATG |
| | GGCATTGCAGGAGTGGTGAGGGACAAGGACACGGAGCTTGGGATTGCTGACGCTGTCATTGCCGTGG |
| | ATGGGATTAACCATGACGTGACCACGGCGTGGGGCGGGGATTATTGGCGTCTGCTGACCCCAGGGGA |
| | CTACATGGTGACTGCCAGTGCCGAGGGCTACCATTCAGTGACACGGAACTGTCGGGTCACCTTTGAA |
| | GAGGGCCCCTTCCCCTGCAATTTCGTGCTCACCAAGACTCCCAAACAGAGGCTGCGCGAGCTGCTGG |
| | CAGCTGGGGCCAAGGTGCCCCCGGACCTTCGCAGGCGCCTGGAGCGGCTAAGGGGACAGAAGGAT |

| | | |
|---|---|---|
| ORF Start: at 1 | | ORF Stop: end of sequence |
| SEQ ID NO: 158 | 714 aa | MW at 79745.4 kD |

| | |
|---|---|
| NOV22e, | APRNSVLGLAQPGTTKVPGSTPALHSSPAQPPAETANGTSEQHVRIRVIKKKKVIMKKRKKLTLTRP |
| CG54007-03 | |
| Protein Sequence | TPLVTAGPLVTPTPAGTLDPAEKQETGCPPLGLESLRVSDSRLEASSSQSFGLGPHRGRLNIQSGLE |
| | DGDLYDGAWCAEEQDADPWFQVDAGHPTRFSGVITQGRNSVWRYDWVTSYKVQFSNDSRTWWGSRNH |
| | SSGMDAVFPANSDPETPVLNLLPEPQVARFIRLLPQTWLQGGAPCLRAEILACPVSDPNDLFLEAPA |
| | SGSSDPLDFQHHNYKAMRKLMKQVQEQCPNITRIYSIGKSYQGLKLYVMEMSDKPGEHELGEPEVRY |
| | VAGMHGNEALGRELLLLLMQFLCHEFLRGNPRVTRLLSEMRIHLLPSMNPDGYEIAYHRGSELVGWA |
| | EGRWNNQSIDLNHNFADLNTPLWEAQDDGKVPHIVPNHHLPLPTYYTLPNATVAPETRAVIKWMKRI |
| | PFVLSANLHGGELVVSYPFDMTRTPWAARELTPTPDDAVFRWLSTVYAGSNLAMQDTSRRPCHSQDF |
| | SVHGNIINGADWHTVPGSMNDFSYLHTNCFEVTVELSCDKFPHENELPQEWENNKDALLTYLEQVRM |
| | GIAGVVRDKDTELGIADAVIAVDGINHDVTTAWGGDYWRLLTPGDYMVTASAEGYHSVTRNCRVTFE |
| | EGPFPCNFVLTKTPKQRLRELLAAGAKVPPDLRRRLERLRGQKD |

| | |
|---|---|
| SEQ ID NO: 159 | 1972 bp |

| | |
|---|---|
| NOV22f, | ATGTGGGGGCTCCTGCTCGCCCTGGCCGCCTTCGCGCCGGCCGTCGGCCCGGCTCTGGGGGCGCCCA |
| CG54007-05 | |
| DNA Sequence | GGAACTCGGTGCTGGGCCTCGCGCAGCCCGGGACCACCAAGGTCCCAGGCTCGACCCCGGCCCTGCA |
| | TAGCAGCCCGGCACAGCCGCCGGCGGAGACAGCTAACGGGACCTCAGAACAGCATGTCCGGATTCGA |
| | GTCATCAAGAAGAAAAAGGTCATTATGAAGAAGCGGAAGAAGCTAACTCTAACTCGCCCCACCCCAC |
| | TGGTGACTGCCGGGCCCCTTGTGACCCCCACTCCAGCAGGGACCCTCGACCCCGCTGAGAAACAAGA |
| | AACAGGCTGTCCTCCTTTGGGTCTGGAGTCCCTGCGAGTTTCAGATAGCCGGCTTGAGGCATCCAGC |
| | AGCCAGTCCTTTGGTCTTGGACCACACCGAGGACGGCTCAACATTCAGTCAGGCCTGGAGGACGGCG |
| | ATCTATATGATGGAGCCTGGTGTGCTGAGGAGCAGGACGCCGATCCATGGTTTCAGGTGGACGCTGG |
| | GCACCCCACCCGCTTCTCGGGTGTTATCACACAGGGCAGAGATCCTGGCCTGCCCAGTCTCAGACCC |
| | CAATGACCTATTCCTTGAGGCCCCTGCGTCGGGATCCTCTGACCCTCTAGACTTTCAGCATCACAAT |
| | TACAAGGCCATGAGGAAGCTGATGAAGCAGGTACAAGAGCAATGCCCCAACATCACCCGCATCTACA |
| | GCATTGGGAAGAGCTACCAGGGCCTGAAGCTGTATGTGATGGAAATGTCGGACAAGCCTGGGGAGCA |
| | TGAGCTGGGGGAGCCTGAGGTGCGCTACGTGGCTGGCATGCATGGGAACGAGGCCCTGGGGCGGGAG |
| | TTGCTTCTGCTCCTGATGCAGTTCCTGTGCCATGAGTTCCTGCGAGGGAACCCACGGGTGACCCGGC |
| | TGCTCTCTGAGATGCGCATTCACCTGCTGCCCTCCATGAACCCTGATGGCTATGAGATCGCCTACCA |
| | CCGGGGTTCAGAGCTGGTGGGCTGGGCCGAGGGCCGCTGGAACAACCAGAGCATCGATCTTAACCAT |
| | AATTTTGCTGACCTCAACACACCACTGTGGGAAGCACAGGACGATGGGAAGGTGCCCCACATCGTCC |
| | CCAACCATCACCTGCCATTGCCCACTTACTACACCCTGCCCAATGCCACCGTGGCTCCTGAAACGCG |
| | GGCAGTAATCAAGTGGATGAAGCGGATCCCCTTTGTGCTAAGTGCCAACCTCCACGGGGGTGAGCTC |
| | GTGGTGTCCTACCCATTCGACATGACTCGCACCCCGTGGGCTGCCCGCGAGCTCACGCCCACACCAG |
| | ATGATGCTGTGTTTCGCTGGCTCAGCACTGTCTATGCTGGCAGTAATCTGGCCATGCAGGACACCAG |
| | CCGCCGACCCTGCCACAGCCAGGACTTCTCCGTGCACGGCAACATCATCAACGGGGCTGACTGGCAC |
| | ACGGTCCCCGGGAGCATGAATGACTTCAGCTACCTACACACCAACTGCTTTGAGGTCACTGTGGAGC |
| | TGTCCTGTGACAAGTTCCCTCACGAGAATGAATTGCCCCAGGAGTGGGAGAACAACAAAGACGCCCT |
| | CCTCACCTACCTGGAGCAGGTGCGCATGGGCATTGCAGGAGTGGTGAGGGACAAGGACACGGAGCTT |
| | GGGATTGCTGACGCTGTCATTGCCGTGGATGGGATTAACCATGACGTGACCACGGCGTGGGGCGGGG |
| | ATTATTGGCGTCTGCTGACCCCAGGGGACTACATGGTGACTGCCAGTGCCGAGGGCTACCATTCAGT |
| | GACACGGAACTGTCGGGTCACCTTTGAAGAGGGCCCCTTCCCCTGCAATTTCGTGCTCACCAAGACT |
| | CCCAAACAGAGGCTGCGCGAGCTGCTGGCAGCTGGGGCCAAGGTGCCCCCGGACCTTCGCAGGCGCC |
| | TGGAGCGGCTAAGGGGACAGAAGGATTGA |

| | | |
|---|---|---|
| ORF Start: ATG at 1 | | ORF Stop: TGA at 607 |
| SEQ ID NO: 160 | 202 aa | MW at 21258.0 kD |

| | |
|---|---|
| NOV22f, | MWGLLLALAAFAPAVGPALGAPRNSVLGLAQPGTTKVPGSTPALHSSPAQPPAETANGTSEQHVRIR |
| CG54007-05 | |
| Protein | VIKKKKVIMKKRKKLTLTRPTPLVTAGPLVTPTPAGTLDPAEKQETGCPPLGLESLRVSDSRLEASS |
| Sequence | |
| | SQSFGLGPHRGRLNIQSGLEDGDLYDGAWCAEEQDADPWFQVDAGHPTRFSGVITQGRDPGLPSLRP |
| | Q |

| | |
|---|---|
| SEQ ID NO: 161 | 2161 bp |

| | |
|---|---|
| NOV22g, | GCCAGATCTGCGCCCAGGAACTCGGTGCTGGGCCTCGCGCAGCCCGGGACCACCAAGGTCCCAGGCT |
| CG54007-07 | |
| DNA Sequence | CGACCCCGGCCCTGCATAGCAGCCCGGCACAGCCGCCGGCGGAGACAGCTAACGGGACCTCAGAACA |
| | GCATGTCCGGATTCGTGTCATCAAGAAGAAAAAGGTCATTATGAAGAAGCGGAAGAAGCTAACTCTA |
| | ACTCGCCCCACCCCACTGGTGACTGCCGGGCCCCTTGTGACCCCCACTCCAGCAGGGACCCTCGACC |
| | CCGCTGAGAAACAAGAAACAGGCTGTCCTCCTTTGGGTCTGGAGTCCCTGCGAGTTTCAGATAGCCG |
| | GCTTGAGGCATCCAGCAGCCAGTCCTTTGGTCTTGGACCACACCGAGGACGGCTCAACATTCAGTCA |
| | GGCCTGGAGGACGGCGATCTATATGATGGAGCCTGGTGTGCTGAGGAGCAGGACGCCGATCCATGGT |
| | TTCAGGTGGACGCTGGGCACCCCACCCGCTTCTCGGGTGTTATCACACAGGGCAGGAACTCTGTCTG |
| | GAGGTATGACTGGGTCACATCATACAAGGTCCAGTTCAGCAATGACAGTCGGACCTGGTGGGGAAGT |
| | AGGAACCACAGCAGTGGGATGGACGCAGTATTTCCTGCCAATTCAGACCCAGAAACTCCAGTGCTGA |
| | ACCTCCTGCCGGAGCCCCAGGTGGCCCGCTTCATTCGCCTGCTGCCCCAGACCTGGCTCCAGGGAGG |
| | CGCGCCTTGCCTCCGGGCAGAGATCCTGGCCTGCCCAGTCTCAGACCCCAATGACCTATTCCTTGAG |
| | GCCCCTGCGTCGGGATCCTCTGACCCTCTAGACTTTCAGCATCACAATTACAAGGCCATGAGGAAGC |
| | TGATGAAGCAGGTACAAGAGCAATGCCCCAACATCACCCGCATCTACAGCATTGGGAAGAGCTACCA |
| | GGGCCTGAAGCTGTATGTGATGGAAATGTCGGACAAGCCTGGGGAGCATGAGCTGGGGGAGCCTGAG |
| | GTGCGCTACGTGGCTGGCATGCATGGGAACGAGGCCCTGGGGCGGGAGTTGCTTCTGCTCCTGATGC |
| | AGTTCCTGTGCCATGAGTTCCTGCGAGGGAACCCACGGGTGACCCGGCTGCTCTCTGAGATGCGCAT |
| | TCACCTGCTGCCCTCCATGAACCCTGATGGCTATGAGATCGCCTACCACCGGGGTTCAGAGCTGGTG |
| | GGCTGGGCCGAGGGCCGCTGGAACAACCAGAGCATCGATCTTAACCATAATTTTGCTGACCTCAACA |
| | CACCACTGTGGGAAGCACAGGACGATGGGAAGGTGCCCCACATCGTCCCCAACCATCACCTGCCATT |
| | GCCCACTTACTACACCCTGCCCAATGCCACCGTGGCTCCTGAAACGCGGGCAGTAATCAAGTGGATG |
| | AAGCGGATCCCCTTTGTGCTAAGTGCCAACCTCCACGGGGGTGAGCTCGTGGTGTCCTACCCATTCG |
| | ACATGACTCGCACCCCGTGGGCTGCCCGCGAGCTCACGCCCACACCAGATGATGCTGTGTTTCGCTG |
| | GCTCAGCACTGTCTATGCTGGCAGTAATCTGGCCATGCAGGACACCAGCCGCCGACCCTGCCACAGC |
| | CAGGACTTCTCCGTGCACGGCAACATCATCAACGGGGCTGACTGGCACACGGTCCCCGGGAGCATGA |
| | ATGACTTCAGCTACCTACACACCAACTGCTTTGAGGTCACTGTGGAGCTGTCCTGTGACAAGTTCCC |
| | TCACGAGAATGAATTGCCCCAGGAGTGGGAGAACAACAAAGACGCCCTCCTCACCTACCTGGAGCAG |
| | GTGCGCATGGGCATTGCAGGAGTGGTGAGGGACAAGGACACGGAGCTTGGGATTGCTGACGCTGTCA |
| | TTGCCGTGGATGGGATTAACCATGACGTGACCACGGCGTGGGGCGGGGATTATTGGCGTCTGCTGAC |
| | CCCAGGGGACTACATGGTGACTGCCAGTGCCGAGGGCTACCATTCAGTGACACGGAACTGTCGGGTC |
| | ACCTTTGAAGAGGGCCCCTTCCCCTGCAATTTCGTGCTCACCAAGACTCCCAAACAGAGGCTGCGCG |
| | AGCTGCTGGCAGCTGGGGCCAAGGTGCCCCCGGACCTTCGCAGGCGCCTGGAGCGGCTAAGGGGACA |
| | GAAGGATCTCGAGGGTG |

| | | |
|---|---|---|
| ORF Start: at 1 | | ORF Stop: at 2161 |
| SEQ ID NO: 162 | 720 aa | MW at 80359.1 kD |

| | |
|---|---|
| NOV22g, | ARSAPRNSVLGLAQPGTTKVPGSTPALHSSPAQPPAETANGTSEQHVRIRVIKKKKVIMKKRKKLTL |
| CG54007-07 | |
| Protein Sequence | TRPTPLVTAGPLVTPTPAGTLDPAEKQETGCPPLGLESLRVSDSRLEASSSQSFGLGPHRGRLNIQS |
| | GLEDGDLYDGAWCAEEQDADPWFQVDAGHPTRFSGVITQGRNSVWRYDWVTSYKVQFSNDSRTWWGS |
| | RNHSSGMDAVFPANSDPETPVLNLLPEPQVARFIRLLPQTWLQGGAPCLRAEILACPVSDPNDLFLE |
| | APASGSSDPLDFQHHNYKAMRKLMKQVQEQCPNITRIYSIGKSYQGLKLYVMEMSDKPGEHELGEPE |
| | VRYVAGMHGNEALGRELLLLLMQFLCHEFLRGNPRVTRLLSEMRIHLLPSMNPDGYEIAYHRGSELV |
| | GWAEGRWNNQSIDLNHNFADLNTPLWEAQDDGKVPHIVPNHHLPLPTYYTLPNATVAPETRAVIKWM |
| | KRIPFVLSANLHGGELVVSYPFDMTRTPWAARELTPTPDDAVFRWLSTVYAGSNLAMQDTSRRPCHS |
| | QDFSVHGNIINGADWHTVPGSMNDFSYLHTNCFEVTVELSCDKFPHENELPQEWENNKDALLTYLEQ |
| | VRMGIAGVVRDKDTELGIADAVIAVDGINHDVTTAWGGDYWRLLTPGDYMVTASAEGYHSVTRNCRV |
| | TFEEGPFPCNFVLTKTPKQRLRELLAAGAKVPPDLRRRLERLRGQKDLEG |

Sequence comparison of the above protein sequences yields the following sequence relationships shown in Table 22B.

TABLE 22B
Comparison of NOV22a against NOV22b through NOV22g.

| | NOV22a Residues/ | Identities/Similarities |
|---|---|---|
| Protein Sequence | Match Residues | for the Matched Region |
| NOV22b | 1 . . . 510 | 510/510 (100%) |
| | 1 . . . 510 | 510/510 (100%) |
| NOV22c | 1 . . . 734 | 734/734 (100%) |
| | 1 . . . 734 | 734/734 (100%) |
| NOV22d | 1 . . . 734 | 734/734 (100%) |
| | 1 . . . 734 | 734/734 (100%) |
| NOV22e | 21 . . . 734 | 714/714 (100%) |
| | 1 . . . 714 | 714/714 (100%) |
| NOV22f | 1 . . . 193 | 192/193 (99%) |
| | 1 . . . 193 | 193/193 (99%) |
| NOV22g | 18 . . . 734 | 715/717 (99%) |
| | 1 . . . 717 | 715/717 (99%) |

Further analysis of the NOV22a protein yielded the following properties shown in Table 22C.

TABLE 22C
Protein Sequence Properties NOV22a

| SignalP | | |
|---|---|---|
| analysis: | Cleavage site between residues 21 and 22 | |
| PSORT II | PSG: | a new signal peptide prediction method |
| analysis: | | N-region: length 0; pos. chg 0; neg. chg 0 |
| | | H-region: length 22; peak value 10.30 |
| | | PSG score: 5.90 |
| | GvH: | von Heijne's method for signal seq. recognition |
| | | GvH score (threshold: −2.1): 0.86 |
| | | possible cleavage site: between 20 and 21 |
| | >>> Seems to have a cleavable signal peptide (1 to 20) | |
| | ALOM: | Klein et al's method for TM region allocation |
| | | Init position for calculation: 21 |
| | | Tentative number of TMS(s) for the threshold 0.5: 0 |
| | | number of TMS(s) . . . fixed |
| | | PERIPHERAL Likelihood = 3.82 (at 613) |
| | | ALOM score: 3.82 (number of TMSs: 0) |
| | MTOP: | Prediction of membrane topology (Hartmann et al.) |
| | | Center position for calculation: 10 |
| | | Charge difference: 1.0 C(2.0) − N(1.0) |
| | | C > N: C-terminal side will be inside |

>>>Caution: Inconsistent mtop result with signal peptide
MITDISC: discrimination of mitochondrial targeting seq

| | | | |
|---|---|---|---|
| R content: | 1 | Hyd Moment | 1.37 |
| Hyd Moment (95): | 2.44 | (75): | |
| D/E content: | 1 | G content: | 6 |
| Score: | −5.91 | S/T content: | 7 |

| Gavel: | prediction of cleavage sites for mitochondrial preseq |
|---|---|
| | R-2 motif at 33 PRN\|SV |
| NUCDISC: discrimination of nuclear localization signals | |
| | pat4: KKKK (5) at 70 |
| | pat4: KKRK (5) at 77 |
| | pat4: KRKK (5) at 78 |
| | pat7: PPDLRRR (3) at 719 |
| | pat7: PDLRRRL (4) at 720 |
| | bipartite: none |
| | content of basic residues: 9.9% |
| | NLS Score: 1.07 |
| KDEL: | ER retention motif in the C-terminus: none |
| ER Membrane Retention Signals: | |
| | KKXX-like motif in the C-terminus: RGQK |
| SKL: | peroxisomal targeting signal in the C-terminus: none |
| PTS2: | 2nd peroxisomal targeting signal: none |
| VAC: | possible vacuolar targeting motif: found |
| | TLPN at 469 |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: cytoplasmic |
| | Reliability: 70.6 |
| COIL: | Lupas's algorithm to detect coiled-coil regions |
| | total: 0 residues |

---------------------------------
Final Results (k = 9/23):

22.2%: extracellular, including cell wall
22.2%: mitochondrial
22.2%: endoplasmic reticulum
11.1%: cytoplasmic
11.1%: vacuolar
11.1%: nuclear

>> prediction for CG54007-06 is exc (k = 9)

A search of the NOV22a protein against the Geneseq database, a proprietary database that contains sequences published in patents and patent publication, yielded several homologous proteins shown in Table 22D.

TABLE 22D
Geneseq Results for NOV22a

| Geneseq | Protein/Organism/Length | NOV22a Residues/ | Identities/Similarities | Expect |
|---|---|---|---|---|
| Identifier | [Patent #, Date] | Match Residues | for the Matched Region | Value |
| AAB47184 | ACPLX protein sequence - | 1 . . . 734 | 734/734 (100%) | 0.0 |
| | Homo sapiens, 734 aa. | 1 . . . 734 | 734/734 (100%) | |
| | [WO200127290-A2, | | | |
| | 19-APR-2001] | | | |
| AAG65917 | Amino acid sequence of | 1 . . . 734 | 734/734 (100%) | 0.0 |
| | GSK gene Id 248602 - Homo | 1 . . . 734 | 734/734 (100%) | |
| | sapiens, 734 aa. | | | |
| | [WO200172961-A2, | | | |
| | 04-OCT-2001] | | | |
| AAB36174 | Human APG04 protein - | 1 . . . 734 | 733/734 (99%) | 0.0 |
| | Homo sapiens, 734 aa. | 1 . . . 734 | 734/734 (99%) | |
| | [US6140098-A, | | | |
| | 31-OCT-2000] | | | |
| AAU29252 | Human PRO polypeptide | 1 . . . 734 | 733/734 (99%) | 0.0 |
| | sequence #229 - Homo | 1 . . . 734 | 733/734 (99%) | |
| | sapiens, 734 aa. | | | |
| | [WO200168848-A2, | | | |
| | 20-SEP-2001] | | | |
| AAB74694 | Human protease and protease | 1 . . . 734 | 733/734 (99%) | 0.0 |
| | inhibitor PPIM-27 - Homo | 1 . . . 734 | 733/734 (99%) | |
| | sapiens, 734 aa. | | | |
| | [WO200110903-A2, | | | |
| | 15-FEB-2001] | | | |

In a BLAST search of public sequence datbases, the NOV22a protein was found to have homology to the proteins shown in the BLASTP data in Table 22E.

TABLE 22E
Public BLASTP Results for NOV22a

| Protein | | | | |
|---|---|---|---|---|
| Accession | | NOV22a Residues/ | Identities/Similarities | Expect |
| Number | Protein/Organism/Length | Match Residues | for the Matched Portion | Value |
| Q96SM3 | Potential carboxypeptidase X | 1 . . . 734 | 733/734 (99%) | 0.0 |
| | precursor (EC 3.4.17.-) | 1 . . . 734 | 733/734 (99%) | |
| | (Metallocarboxypeptidase | | | |
| | CPX-1) - Homo sapiens | | | |
| | (Human), 734 aa. | | | |
| Q9Z100 | Potential carboxypeptidase X | 1 . . . 733 | 622/733 (84%) | 0.0 |
| | precursor (EC 3.4.17.-) | 1 . . . 722 | 661/733 (89%) | |
| | (Metallocarboxypeptidase | | | |
| | CPX-1) - Mus musculus | | | |
| | (Mouse), 722 aa. | | | |
| Q8N2E1 | Hypothetical protein | 1 . . . 465 | 464/465 (99%) | 0.0 |
| | HEMBA1005833 - Homo | 1 . . . 465 | 464/465 (99%) | |
| | sapiens (Human), 477 aa. | | | |
| Q8N2F1 | Hypothetical protein | 305 . . . 734 | 430/430 (100%) | 0.0 |
| | HEMBA1002913 - Homo | 1 . . . 430 | 430/430 (100%) | |
| | sapiens (Human), 430 aa. | | | |
| Q8N436 | Hypothetical protein - Homo | 48 . . . 733 | 376/695 (54%) | 0.0 |
| | sapiens (Human), 807 aa | 113 . . . 802 | 480/695 (68%) | |
| | (fragment). | | | |

PFam analysis predicts that the NOV22a protein contains the domains shown in the Table 22F.

TABLE 22F
Domain Analysis of NOV22a

| | NOV22a Match | Identities/Similarities | Expect |
|---|---|---|---|
| Pfam Domain | Region | for the Matched Region | Value |
| F5_F8_type_C | 117 . . . 271 | 73/168 (43%) | 2.4e−65 |
| | | 133/168 (79%) | |
| Zn_carbOpept | 299 . . . 416 | 39/123 (32%) | 2.5e−19 |
| | | 89/123 (72%) | |
| Zn_carbOpept | 475 . . . 675 | 46/212 (22%) | 1.5e−27 |
| | | 160/212 (75%) | |

### Example 23

The NOV23 clone was analyzed, and the nucleotide and encoded polypeptide sequences are shown in Table 23A.

TABLE 23A
NOV23 Sequence Analysis

| | |
|---|---|
| SEQ ID NO: 163 | 1371 bp |

| | |
|---|---|
| NOV23a, | ACCATGGAGCTGGCACTGCGGCGCTCTCCCGTCCCGCGGTGGTTGCTGCTGCTGCCGCTGCTGCTGG |
| CG55078-04 | |
| DNA Sequence | GCCTGAACGCAGGAGCTGTCATTGACTGGCCCACAGAGGAGGGCAAGGAAGTATGGGATTATGTGAC |
| | GGTCCGCAAGGATGCCTACATGTTCTGGTGGCTCTATTATGCCACCAACTCCTGCAAGAACTTCTCA |
| | GAACTGCCCCTGGTCATGTGGCTTCAGGGCGGTCCAGGCGGTTCTAGCACTGGATTTGGAAACTTTG |
| | AGGAAATTGGGCCCCTTGACAGTGATCTCAAACCACGGAAAACCACCTGGCTCCAGGCTGCCAGTCT |
| | CCTATTTGTGGATAATCCCGTGGGCACTGGGTTCAGTTATGTGAATGGTAGTGGTGCCTATGCCAAG |
| | GACCTAGCTATGGTGGCTTCAGACATGATGGTTCTCCTGAAGACCTTCTTCAGTTGCCACAAAGAAT |
| | TCCAGACAGTTCCATTCTACATTTTCTCAGAGTCCTATGGAGGAAAAATGGCAGCTGGCATTGGTCT |
| | AGAGCTTTATAAGGCCATTCAGCGAGGGACCATCAAGTGCAACTTTGCGGGGGTTGCCTTGGGTGAT |
| | TCCTGGATCTCCCCTGTTGATTCGGTGCTCTCCTGGGGACCTTACCTGTACAGCATGTCTCTTCTCG |
| | AAGACAAAGGTCTGGCAGAGGTGTCTAAGGTTGCAGAGCAAGTACTGAATGCCGTAAATAAGGGGCT |
| | CTACAGAGAGGCCACAGAGCTGTGGGGGAAAGCAGAAATGATCATTGAACAGAACACAGATGGGGTG |
| | AACTTCTATAACATCTTAACTAAAAGCACTCCCACGTCTACAATGGAGTCGAGTCTAGAATTCACAC |
| | AGAGCCACCTAGTTTGTCTTTGTCAGCGCCACGTGAGACACCTACAACGAGATGCCTTAAGCCAGCT |
| | CATGAATGGCCCCATCAGAAAGAAGCTCAAAATTATTCCTGAGGATCAATCCTGGGGAGGCCAGGCT |
| | ACCAACGTCTTTGTGAACATGGAGGAGGACTTCATGAAGCCAGTCATTAGCATTGTGGACGAGTTGC |
| | TGGAGGCAGGGATCAACGTGACGGTGTATAATGGACAGCTGGATCTCATCGTAGATACCATGGGTCA |
| | GGAGGCCTGGGTGCGGAAACTGAAGTGGCCAGAACTGCCTAAATTCAGTCAGCTGAAGTGGAAGGCC |
| | CTGTACAGTGACCCTAAATCTTTGGAAACATCTGCTTTTGTCAAGTCCTACAAGAACCTTGCTTTCT |
| | ACTGGATTCTGAAAGCTGGTCATATGGTTCCTTCTGACCAAGGGGACATGGCTCTGAAGATGATGAG |
| | ACTGGTGACTCAGCAAGAATAGCCGCGGCGC |

| | | |
|---|---|---|
| ORF Start: at 1 | | ORF Stop: TAG at 1360 |
| SEQ ID NO: 164 | 453 aa | MW at 50931.3 kD |

| | |
|---|---|
| NOV23a, | TMELALRRSPVPRWLLLLPLLLGLNAGAVIDWPTEEGKEVWDYVTVRKDAYMFWWLYYATNSCKNFS |
| CG55078-04 | |
| Protein | ELPLVMWLQGGPGGSSTGFGNFEEIGPLDSDLKPRKTTWLQAASLLFVDNPVGTGFSYVNGSGAYAK |
| Sequence | |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLVCLCQRHVRHLQRDALSQLMNGPIRKKLKIIPEDQSWGGQA |
| | TNVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQE |

| | |
|---|---|
| SEQ ID NO: 165 | 1650 bp |

| | |
|---|---|
| NOV23b, | GCCTGTTGCTGATGCTGCCGTGCGGTACTTGTCATGGAGCTGGCACTGCGGCGCTCTCCCGTCCCGC |
| CG55078-01 | |
| DNA Sequence | GGTGGTTGCTGCTGCTGCCGCTGCTGCTGGGCCTGAACGCAGGAGCTGTCATTGACTGGCCCACAGA |
| | GGAGGGCAAGGAAGTATGGGATTATGTGACGGTCCGCAAGGATGCCTACATGTTCTGGTGGCTCTAT |
| | TATGCCACCAACTCCTGCAAGAACTTCTCAGAACTGCCCCTGGTCATGTGGCTTCAGGGCGGTCCAG |
| | GCGGTTCTAGCACTGGATTTGGAAACTTTGAGGAAATTGGGCCCCTTGACAGTGATCTCAAACCACG |
| | GAAAACCACCTGGCTCCAGGCTGCCAGTCTCCTATTTGTGGATAATCCCGTGGGCACTGGGTTCAGT |
| | TATGTGAATGGTAGTGGTGCCTATGCCAAGGACCTGGCTATGGTGGCTTCAGACATGATGGTTCTCC |
| | TGAAGACCTTCTTCAGTTGCCACAAAGAATTCCAGACAGTTCCATTCTACATTTTCTCAGAGTCCTA |
| | TGGAGGAAAAATGGCAGCTGGCATTGGTCTAGAGCTTTATAAGGCCATTCAGCGAGGGACCATCAAG |
| | TGCAACTTTGCGGGGGTTGCCTTGGGTGATTCCTGGATCTCCCCTGTTGATTCGGTGCTCTCCTGGG |
| | GACCTTACCTGTACAGCATGTCTCTTCTCGAAGACAAAGGTCTGGCAGAGGTGTCTAAGGTTGCAGA |
| | GCAAGTACTGAATGCCGTAAATAAGGGGCTCTACAGAGAGGCCACAGAGCTGTGGGGGAAAGCAGAA |
| | ATGATCATTGAACAGAACACAGATGGGGTGAACTTCTATAACATCTTAACTAAAAGCACTCCCACGT |
| | CTACAATGGAGTCGAGTCTAGAATTCACACAGAGCCACCTAGTTTGTCTTTGTCAGCGCCACGTGAG |
| | ACACCTACAACGAGATGCCTTAAGCCAGCTCATGAATGGCCCCATCAGAAAGAAGCTCAAAATTATT |
| | CCTGAGGATCAATCCTGGGGAGGCCAGGCTACCAACGTCTTTGTGAACATGGAGGAGGACTTCATGA |
| | AGCCAGTCATTAGCATTGTGGACGAGTTGCTGGAGGCAGGGATCAACGTGACGGTGTATAATGGACA |
| | GCTGGATCTCATCGTAGATACCATGGGTCAGGAGGCCTGGGTGCGGAAACTGAAGTGGCCAGAACTG |
| | CCTAAATTCAGTCAGCTGAAGTGGAAGGCCCTGTACAGTGACCCTAAATCTTTGGAAACATCTGCTT |
| | TTGTCAAGTCCTACAAGAACCTTGCTTTCTACTGGATTCTGAAAGCTGGTCATATGGTTCCTTCTGA |
| | CCAAGGGGACATGGCTCTGAAGATGATGAGACTGGTGACTCAGCAAGAATAGGATGGATGGGGCTGG |
| | AGATGAGCTGGTTTGGCCTTGGGGCACAGAGCTGAGCTGAGGCCGCTGAAGCTGTAGGAAGCGCCAT |
| | TCTTCCCTGTATCTAACTGGGGCTGTGATCAAGAAGGTTCTGACCAGCTTCTGCAGAGGATAAAATC |
| | ATTGTCTCTGGAGGCAATTTGGAAATTATTTCTGCTTCTTAAAAAAACCTAAGATTTTTTAAAAAAT |
| | TGATTTGTTTTGATCAAAATAAAGGATGATAATAGATATTAA |

| | | |
|---|---|---|
| ORF Start: ATG at 34 | | ORF Stop: TAG at 1390 |
| SEQ ID NO: 166 | 452 aa | MW at 50830.2 kD |

| | |
|---|---|
| NOV23b, | MELALRRSPVPRWLLLLPLLLGLNAGAVIDWPTEEGKEVWDYVTVRKDAYMFWWLYYATNSCKNFSE |
| CG55078-01 | |
| Protein Sequence | LPLVMWLQGGPGGSSTGFGNFEEIGPLDSDLKPRKTTWLQAASLLFVDNPVGTGFSYVNGSGAYAKD |
| | LAMVASDMMVLLKTFFSCHKEFQTVPFYIFSESYGGKMAAGIGLELYKAIQRGTIKCNFAGVALGDS |
| | WISPVDSVLSWGPYLYSMSLLEDKGLAEVSKVAEQVLNAVNKGLYREATELWGKAEMIIEQNTDGVN |
| | FYNILTKSTPTSTMESSLEFTQSHLVCLCQRHVRHLQRDALSQLMNGPIRKKLKIIPEDQSWGGQAT |
| | NVFVNMEEDFMKPVISIVDELLEAGINVTVYNGQLDLIVDTMGQEAWVRKLKWPELPKFSQLKWKAL |
| | YSDPKSLETSAFVKSYKNLAFYWILKAGHMVPSDQGDMALKMMRLVTQQE |

| | |
|---|---|
| SEQ ID NO: 167 | 1644 bp |

| | |
|---|---|
| NOV23c, | GCCTGTTGCTGATGCTGCCGTGCGGTACTTGTCATGGAGCTGGCACTGCGGCGCTCTCCCGTCCCGC |
| CG55078-03 | |
| DNA Sequence | GGTGGTTGCTGCTGCTGCCGCTGCTGCTGGGCCTGAACGCAGGAGCTGTCATTGACTGGCCCACAGA |
| | GGAGGGCAAGGAAGTATGGGATTATGTGACGGTCCGCAAGGATGCCTACATGTTCTGGTGGCTCTAT |
| | TATGCCACCAACTCCTGCAAGAACTTCTCAGAACTGCCCCTGGTCATGTGGCTTCAGGGCGGTCCAG |
| | GCGGTTCTAGCACTGGATTTGGAAACTTTGAGGAAATTGGGCCCCTTGACAGTGATCTCAAACCACG |
| | GAAAACCACCTGGCTCCAGGCTGCCAGTCTCCTATTTGTGGATAATCCCGTGGGCACTGGGTTCAGT |
| | TATGTGAATGGTAGTGGTGCCTATGCCAAGGACCTGGCTATGGTGGCTTCAGACATGATGGTTCTCC |
| | TGAAGACCTTCTTCAGTTGCCACAAAGAATTCCAGACAGTTCCATTCTACATTTTCTCAGAGTCCTA |
| | TGGAGGAAAAATGGCAGCTGGCATTGGTCTAGAGCTTTATAAGGCCATTCAGCGAGGGACCATCAAG |
| | TGCAACTTTGCGGGGGTTGCCTTGGGTGATTCCTGGATCTCCCCTGTTGATTCGGTGCTCTCCTGGG |
| | GACCTTACCTGTACAGCATGTCTCTTCTCGAAGACAAAGGTCTGGCAGAGGTGTCTAAGGTTGCAGA |
| | GCAAGTACTGAATGCCGTAAATAAGGGGCTCTACAGAGAGGCCACAGAGCTGTGGGGGAAAGCAGAA |
| | ATGATCATTGAACAGAACACAGATGGGGTGAACTTCTATAACATCTTAACTAAAAGCACTCCCACGT |
| | CTACAATGGAGTCGAGTCTAGAATTCACACAGAGCCACCTAGTTTGTCTTTGTCAGCGCCACGTGAG |
| | ACACCTACAACGAGATGCCTTAAGCCAGCTCATGAATGGCCCCATCAGAAAGAAGCTCAAAATTATT |
| | CCTGAGGATCAATCCTGGGGAGGCCAGGCTACCAACGTCTTTGTGAACATGGAGGAGGACTTCATGA |
| | AGCCAGTCATTAGCATTGTGGACGAGTTGCTGGAGGCAGGGATCAACGTGACGGTGTATAATGGACA |
| | GCTGGATCTCATCGTAGATACCATGGGTCAGGAGGCCTGGGTGCGGAAACTGAAGTGGCCAGAACTG |
| | CCTAAATTCAGTCAGCTGAAGTGGAAGGCCCTGTACAGTGACCCTAAATCTTTGGAAACATCTGCTT |
| | TTGTCAAGTCCTACAAGAACCTTGCTTTCTACTGGATTCTGAAAGCTGGTCATATGGTTCCTTCTGA |
| | CCAAGGGGACATGGCTCTGAAGATGATGAGACTGGTGACTCAGCAAGAATAGGATGGATGGGGCTGG |
| | AGATGAGCTGGTTTGGCCTTGGGGCACAGAGCTGAGCTGAGGCCGCTGAAGCTGTAGGAAGCGCCAT |
| | TCTTCCCTGTATCTAACTGGGGCTGTGATCAAGAAGGTTCTGACCAGCTTCTGCAGAGGATAAAATC |
| | ATTGTCTCTGGAGGCAATTTGGAAATTATTTCTGCTTCTTAAAAAAACCTAAGATTTTTTAAAAAAT |
| | TGATTTGTTTTGATCAAAATAAAGGATGATAATAGA |

| | | |
|---|---|---|
| ORF Start: ATG at 34 | | Stop: TAG at 1390 |
| SEQ ID NO: 168 | 452 aa | MW at 50830.2 kD |

| | |
|---|---|
| NOV23c, | MELALRRSPVPRWLLLLPLLLGLNAGAVIDWPTEEGKEVWDYVTVRKDAYMFWWLYYATNSCKNFSE |
| CG55078-03 | |
| Protein | LPLVMWLQGGPGGSSTGFGNFEEIGPLDSDLKPRKTTWLQAASLLFVDNPVGTGFSYVNGSGAYAKD |
| Sequence | |
| | LAMVASDMMVLLKTFFSCHKEFQTVPFYIFSESYGGKMAAGIGLELYKAIQRGTIKCNFAGVALGDS |
| | WISPVDSVLSWGPYLYSMSLLEDKGLAEVSKVAEQVLNAVNKGLYREATELWGKAEMIIEQNTDGVN |
| | FYNILTKSTPTSTMESSLEFTQSHLVCLCQRHVRHLQRDALSQLMNGPIRKKLKIIPEDQSWGGQAT |
| | NVFVNMEEDFMKPVISIVDELLEAGINVTVYNGQLDLIVDTMGQEAWVRKLKWPELPKFSQLKWKAL |
| | YSDPKSLETSAFVKSYKNLAFYWILKAGHMVPSDQGDMALKMMRLVTQQE |

| | |
|---|---|
| SEQ ID NO: 169 | 1381 bp |

| | |
|---|---|
| NOV23d, | CACCATGGAGCTGGCACTGCGGCGCTCTCCCGTCCCGCGGTGGTTGCTGCTGCTGCCGCTGCTGCTG |
| 171094334 DNA | |
| Sequence | GGCCTGAACGCAGGAGCTGTCATTGACTGGCCCACAGAGGAGGGCAAGGAAGTATGGGATTATGTGA |
| | CGGTCCGCAAGGATGCCTACATGTTCTGGTGGCTCTATTATGCCACCAACTCCTGCAAGAACTTCTC |
| | AGAACTGCCCCTGGTCATGTGGCTTCAGGGCGGTCCAGGCGGTTCTAGCACTGGATTTGGAAACTTT |
| | GAGGAAATTGGGCCCCTTGACAGTGATCTCAAACCACGGAAAACCACCTGGCTCCAGGCTGCCAGTC |
| | TCCTATTTGTGGATAATCCCGTGGGCACTGGGTTCAGTTATGTGAATGGTAGTGGTGCCTATGCCAA |
| | GGACCTGGCTATGGTGGCTTCAGACATGATGGTTCTCCTGAAGACCTTCTTCAGTTGCCACAAAGAA |
| | TTCCAGACAGTTCCATTCTACATTTTCTCAGAGTCCTATGGAGGAAAAATGGCAGCTGGCATTGGTC |
| | TAGAGCTTTATAAGGCCATTCAGCGAGGGACCATCAAGTGCAACTTTGCGGGGGTTGCCTTGGGTGA |
| | TTCCTGGATCTCCCCTGTTGATTCGGTGCTCTCCTGGGGACCTTACCTGTACAGCATGTCTCTTCTC |
| | GAAGACAAAGGTCTGGCAGAGGTGTCTAAGGTTGCAGAGCAAGTACTGAATGCCGTAAATAAGGGGC |
| | TCTACAGAGAGGCCACAGAGCTGTGGGGGAAAGCAGAAATGATCATTGAACAGAACACAGATGGGGT |
| | GAACTTCTATAACATCTTAACTAAAAGCACTCCCACGTCTACAATGGAGTCGAGTCTAGAATTCACA |
| | CAGAGCCACCTAGTTTGTCTTTGTCAGCGCCACGTGAGACACCTACAACGAGATGCCTTAAGCCAGC |
| | TCATGAATGGCCCCATCAGAAAGAAGCTCAAAATTATTCCTGAGGATCAATCCTGGGGAGGCCAGGC |
| | TACCAACGTCTTTGTGAACATGGAGGAGGACTTCATGAAGCCAGTCATTAGCATTGTGGACGAGTTG |
| | CTGGAGGCAGGGATCAACGTGACGGTGTATAATGGACAGCTGGATCTCATCGTAGATACCATGGGTC |
| | AGGAGGCCTGGGTGCGGAAACTGAAGTGGCCAGAACTGCCTAAATTCAGTCAGCTGAAGTGGAAGGC |
| | CCTGTACAGTGACCCTAAATCTTTGGAAACATCTGCTTTTGTCAAGTCCTACAAGAACCTTGCTTTC |
| | TACTGGATTCTGAAAGCTGGTCATATGGTTCCTTCTGACCAAGGGGACATGGCTCTGAAGATGATGA |
| | GACTGGTGACTCAGCAAGAACACCATCACCACCATCACTAG |

| | | |
|---|---|---|
| ORF Start: at 2 | | ORF Stop: TAG at 1379 |
| SEQ ID NO: 170 | 459 aa | MW at 51754.2 kD |

| | |
|---|---|
| NOV23d, | TMELALRRSPVPRWLLLLPLLLGLNAGAVIDWPTEEGKEVWDYVTVRKDAYMFWWLYYATNSCKNFS |
| 171094334 | |
| Protein Sequence | ELPLVMWLQGGPGGSSTGFGNFEEIGPLDSDLKPRKTTWLQAASLLFVDNPVGTGFSYVNGSGAYAK |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLVCLCQRHVRHLQRDALSQLMNGPIRKKLKIIPEDQSWGGQA |
| | TNVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQEHHHHHH |

| | |
|---|---|
| SEQ ID NO: 171 | 1267 bp |

| | |
|---|---|
| NOV23e, | CCACCATGGAGCTGGCACTGCGGCGCTCTCCCGTCCCGCGGTGGTTGCTGCTGCTGCCGCTGCTGCT |
| 171095197 DNA | |
| Sequence | GGGCCTGAACACAGGAGCTGTCATTGACTGGCCCACAGAGGAGGGCAAGGAAGTATGGGATTATGTG |
| | ACGGTCCGCAAGGATGCCTACATGTTCTGGTGGCTCTATTATGCCACCAACTCCTGCAAGAACTTCT |
| | CAGAACTGCCCCTGGTCATGTGGCTTCAGGGCGGTCCAGGCGGTTCTAGCACTGGATTTGGAAACTT |
| | TGAGGAAATTGGGCCCCTTGACAGTGATCTCAAACCACGGAAAACCACCTGGCTCCAGGCTGCCAGT |
| | CTCCTATTTGTGGATAATCCCGTGGGCACTGGGTTCAGTTATGTGAATGGTAGTGGTGCCTATGCCA |
| | AGGACCTGGCTATGGTGGCTTCAGACATGATGGTTCTCCTGAAGACCTTCTTCAGTTGCCACAAAGA |
| | ATTCCAGACAGTTCCATTCTACATTTTCTCAGAGTCCTATGGAGGAAAAATGGCAGCTGGCATTGGT |
| | CTAGAGCTTTATAAGGCCATTCAGCGAGGGACCATCAAGTGCAACTTTGCGGGGGTTGCCTTGGGTG |
| | ATTCCTGGATCTCCCCTGTTGATTCGGTGCTCTCCTGGGGACCTTACCTGTACAGCATGTCTCTTCT |
| | CGAAGACAAAGGTCTGGCAGAGGTGTCTAAGGTTGCAGAGCAAGTACTGAATGCCGTAAATAAGGGG |
| | CTCTACAGAGAGGCCACAGAGCTGTGGGGGAAAGCAGAAATGATCATTGAACAGAACACAGATGGGG |
| | TGAACTTCTATAACATCTTAACTAAAAGCACTCCCACGTCTACAATGGAGTCGAGTCTAGAATTCAC |
| | ACAGAGCCACCTAGGCCAGGCTACCAACGTCTTTGTGAACATGGAGGAGGACTTCATGAAGCCAGTC |
| | ATTAGCATTGTGGACGAGTTGCTGGAGGCAGGGATCAACGTGACGGTGTATAATGGACAGCTGGATC |
| | TCATCGTAGATACCATGGGTCAGGGGGCCTGGGTGCGGAAACTGAAGTGGCCAGAACTGCCTAAATT |
| | CAGTCAGCTGAAGTGGAAGGCCCTGTACAGTGACCCTAAATCTTTGGAAACATCTGCTTTTGTCAAG |
| | TCCTACAAGAACCTTGCTTTCTACTGGATTCTGAAAGCTGGTCATATGGTTCCTTCTGACCAAGGGG |
| | ACATGGCTCTGAAGATGATGAGACTGGTGACTCAGCAAGAACACCATCACCACCATCACTA |

| | | |
|---|---|---|
| ORF Start: at 3 | | ORF Stop: end of sequence |
| SEQ ID NO: 172 | 422 aa | MW at 47261.9kD |

| | |
|---|---|
| NOV23e, | TMELALRRSPVPRWLLLLPLLLGLNTGAVIDWPTEEGKEVWDYVTVRKDAYMFWWLYYATNSCKNFS |
| 171095197 | |
| Protein | ELPLVMWLQGGPGGSSTGFGNFEEIGPLDSDLKPRKTTWLQAASLLFVDNPVGTGFSYVNGSGAYAK |
| Sequence | |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLGQATNVFVNMEEDFMKPVISIVDELLEAGINVTVYNGQLDL |
| | IVDTMGQGAWVRKLKWPELPKFSQLKWKALYSDPKSLETSAFVKSYKNLAFYWILKAGHMVPSDQGD |
| | MALKMMRLVTQQEHHHHHHX |

| | |
|---|---|
| SEQ ID NO: 173 | 1733 bp |

| | |
|---|---|
| NOV23f, | ATCGTAACAACTCCGCCCCATTGACGCAAATGGGCGGTAGGCGTGTACGGTGGGAGGTCTATATAAG |
| 214374121 | |
| DNA Sequence | CAGAGCTCTCTGGCTAACTAGAGAACCCACTGCTTACTGGCTTATCGAAATTAATACGACTCACTAT |
| | AGGGAGACCCAAGCTGGCTAGCGTTTAAACTTAAGCTTGGTACCGAGCTCGGATCCCCACCATGGAG |
| | CTGGCACTGCGGCGCTCTCCCGTCCCGCGGTGGTTGCTGCTGCTGCCGCTGCTGCTGGGCCTGAACG |
| | CAGGAGCTGTCATTGACTGGCCCACAGAGGAGGGCAAGGAAGTATGGGATTATGTGACGGTCCGCAA |
| | GGATGCCTACATGTTCTGGTGGCTCTATTATGCCACCAACTCCTGCAAGAACTTCTCAGAACTGCCC |
| | CTGGTCATGTGGCTTCAGGGCGGTCCAGGCGGTTCTAGCACTGGATTTGGAAACTTTGAGGAAATTG |
| | GGCCCCTTGACAGTGATCTCAAACCACGGAAAACCACCTGGCTCCAGGCTGCCAGTCTCCTATTTGT |
| | GGATAATCCCGTGGGCACTGGGTTCAGTTATGTGAATGGTAGTGGTGCCTATGCCAAGGACCTGGCT |
| | ATGGTGGCTTCAGACATGATGGTTCTCCTGAAGACCTTCTTCAGTTGCCACAAAGAATTCCAGACAG |
| | TTCCATTCTACATTTTCTCAGAGTCCTATGGAGGAAAAATGGCAGCTGGCATTGGTCTAGAGCTTTA |
| | TAAGGCCATTCAGCGAGGGACCATCAAGTGCAACTTTGCGGGGGTTGCCTTGGGTGATTCCTGGATC |
| | TCCCCTGTTGATTCGGTGCTCTCCTGGGGACCTTACCTGTACAGCATGTCTCTTCTCGAAGACAAAG |
| | GTCTGGCAGAGGTGTCTAAGGTTGCAGAGCAAGTACTGAATGCCGTAAATAAGGGGCTCTACAGAGA |
| | GGCCACAGAGCTGTGGGGGAAAGCAGAAATGATCATTGAACAGAACACAGATGGGGTGAACTTCTAT |
| | AACATCTTAACTAAAAGCACTCCCACGTCTACAATGGAGTCGAGTCTAGAATTCACACAGAGCCACC |
| | TAGTTTGTCTTTGTCAGCGCCACGTGAGACACCTACAACGAGATGCCTTAAGCCAGCTCATGAATGG |
| | CCCCATCAGAAAGAAGCTCAAAATTATTCCTGAGGATCAATCCTGGGGAGGCCAGGCTACCAACGTC |
| | TTTGTGAACATGGAGGAGGACTTCATGAAGCCAGTCATTAGCATTGTGGACGAGTTGCTGGAGGCAG |
| | GGATCAACGTGACGGTGTATAATGGACAGCTGGATCTCATCGTAGATACCATGGGTCAGGAGGCCTG |
| | GGTGCGGAAACTGAAGTGGCCAGAACTGCCTAAATTCAGTCAGCTGAAGTGGAAGGCCCTGTACAGT |
| | GACCCTAAATCTTTGGAAACATCTGCTTTTGTCAAGTCCTACAAGAACCTTGCTTTCTACTGGATTC |
| | TGAAAGCTGGTCATATGGTTCCTTCTGACCAAGGGGACATGGCTCTGAAGATGATGAGACTGGTGAC |
| | TCAGCAAGAACACCATCACCACCATCACTAGGCGGCCGCTCGAGTCTAGAGGGCCCGTTTAAACCCG |
| | CTGATCAGCCTCGACTGTGCCTTCTAGTTGCCAGCCATCTGTTGTTTGCCCCTCCCCCGTGCCTTCC |
| | TTGACCCTGGAAGGTGCACTCCCACTGTCCTTTCTAATAAATGAGGAAATTGATCGCA |

| | | |
|---|---|---|
| ORF Start: at 169 | | ORF Stop: TAG at 1570 |
| SEQ ID NO: 174 | 467 aa | MW at 52768.3 kD |

| | |
|---|---|
| NOV23f | AWYRARIPTMELALRRSPVPRWLLLLPLLLGLNAGAVIDWPTEEGKEVWDYVTVRKDAYMFWWLYYA |
| 214374121 | |
| Protein | TNSCKNFSELPLVMWLQGGPGGSSTGFGNFEEIGPLDSDLKPRKTTWLQAASLLFVDNPVGTGFSYV |
| Sequence | |
| | NGSGAYAKDLAMVASDMMVLLKTFFSCHKEFQTVPFYIFSESYGGKMAAGIGLELYKAIQRGTIKCN |
| | FAGVALGDSWISPVDSVLSWGPYLYSMSLLEDKGLAEVSKVAEQVLNAVNKGLYREATELWGKAEMI |
| | IEQNTDGVNFYNILTKSTPTSTMESSLEFTQSHLVCLCQRHVRHLQRDALSQLMNGPIRKKLKIIPE |
| | DQSWGGQATNVFVNMEEDFMKPVISIVDELLEAGINVTVYNGQLDLIVDTMGQEAWVRKLKWPELPK |
| | FSQLKWKALYSDPKSLETSAFVKSYKNLAFYWILKAGHMVPSDQGDMALKMMRLVTQQEHHHHHH |

| | |
|---|---|
| SEQ ID NO: 175 | 1364 bp |

| | |
|---|---|
| NOV23g, | CACCATGGAGCTGGCACTGCGGCGCTCTCCCGTCCCGCGGTGGTTGCTGCTGCTGCCGCTGCTGCTG |
| 171095146 DNA | |
| Sequence | GGCCTGAACGCAGGAGCTGTCATTGACTGGCCCACAGAGGAGGGCAAGGAAGTATGGGATTATGTGA |
| | CGGTCCGCAAGGATGCCTACATGTTCTGGTGGCTCTATTATGCCACCAACTCCTGCAAGAACTTCTC |
| | AGAACTGCCCCTGGTCATGTGGCTTCAGGGCGGTCCAGGCGGTTCTAGCACTGGATTTGGAAACTTT |
| | GAGGAAATTGGGCCCCTTGACAGTGATCTCAAACCACGGAAAACCACCTGGCTCCAGGCTGCCAGTC |
| | TCCTATTTGTGGATAATCCCGTGGGCACTGGGTTCAGTTATGTGAATGGTAGTGGTGCCTATGCCAA |
| | GGACCTAGCTATGGTGGCTTCAGACATGATGGTTCTCCTGAAGACCTTCTTCAGTTGCCACAAAGAA |
| | TTCCAGACAGTTCCATTCTACATTTTCTCAGAGTCCTATGGAGGAAAAATGGCAGCTGGCATTGGTC |
| | TAGAGCTTTATAAGGCCATTCAGCGAGGGACCATCAAGTGCAACTTTGCGGGGGTTGCCTTGGGTGA |
| | TTCCTGGATCTCCCCTGTTGATTCGGTGCTCTCCTGGGGACCTTACCTGTACAGCATGTCTCTTCTC |
| | GAAGACAAAGGTCTGGCAGAGGTGTCTAAGGTTGCAGAGCAAGTACTGAATGCCGTAAATAAGGGGC |
| | TCTACAGAGAGGCCACAGAGCTGTGGGGGAAAGCAGAAATGATCATTGAACAGAACACAGATGGGGT |
| | GAACTTCTATAACATCTTAACTAAAAGCACTCCCACGTCTACAATGGAGTCGAGTCTAGAATTCACA |
| | CAGAGCCACCTAGTTTGTCTTTGTCAGCGCCACGTGAGACACCTACAACGAGATGCCTTAAGCCAGC |
| | TCATGAATGGCCCCATCAGAAAGAAGCTCAAAATTATTCCTGAGGATCAATCCTGGGGAGGCCAGGC |
| | TACCAACGTCTTTGTGAACATGGAGGAGGACTTCATGAAGCCAGTCATTAGCATTGTGGACGAGTTG |
| | CTGGAGGCAGGGATCAACGTGACGGTGTATAATGGACAGCTGGATCTCATCGTAGATACCATGGGTC |
| | AGGAGGCCTGGGTGCGGAAACTGAAGTGGCCAGAACTGCCTAAATTCAGTCAGCTGAAGTGGAAGGC |
| | CCTGTACAGTGACCCTAAATCTTTGGAAACATCTGCTTTTGTCAAGTCCTACAAGAACCTTGCTTTC |
| | TACTGGATTCTGAAAGCTGGTCATATGGTTCCTTCTGACCAAGGGGACATGGCTCTGAAGATGATGA |
| | GACTGGTGACTCAGCAAGAATAGC |

| | | |
|---|---|---|
| ORF Start: at 2 | | ORF Stop: TAG at 1361 |
| SEQ ID NO: 176 | 453 aa | MW at 50931.3 kD |

| | |
|---|---|
| NOV23g, | TMELALRRSPVPRWLLLLPLLLGLNAGAVIDWPTEEGKEVWDYVTVRKDAYMFWWLYYATNSCKNFS |
| 171095146 | |
| Protein Sequence | ELPLVMWLQGGPGGSSTGFGNFEEIGPLDSDLKPRKTTWLQAASLLFVDNPVGTGFSYVNGSGAYAK |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLVCLCQRHVRHLQRDALSQLMNGPIRKKLKIIPEDQSWGGQA |
| | TNVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQE |

| | |
|---|---|
| SEQ ID NO: 177 | 1364 bp |

| | |
|---|---|
| NOV23h, | CACCATGGAGCTGGCACTGCGGCGCTCTCCCGTCCCGCGGTGGTTGCTGCTGCTGCCGCTGCTGCTG |
| 171095500 DNA | |
| Sequence | GGCCTGAACGCAGGAGCTGTCATTGACTGGCCCACAGAGGAGGGCAAGGAAGTATGGGATTATGTGA |
| | CGGTCCGCAAGGATGCCTACATGTTCTGGTGGCTCTATTATGCCACCAACTCCTGCAAGAACTTCTC |
| | AGAACTGCCCCTGGTCATGTGGCTTCAGGGCGGTCCAGGCGGTTCTAGCACTGGATTTGGAAACTTT |
| | GAGGAAATTGGGCCCCTTGACAGTGATCTCAAACCACGGAAAACCACCTGGCTCCAGGCTGCCAGTC |
| | TCCTATTTGTGGATAATCCCGTGGGCACTGGGTTCAGTTATGTGAATGGTAGTGGTGCCTATGCCAA |
| | GGACCTAGCTATGGTGGCTTCAGACATGATGGTTCTCCTGAAGACCTTCTTCAGTTGCCACAAAGAA |
| | TTCCAGACAGTTCCATTCTACATTTTCTCAGAGTCCTATGGAGGAAAAATGGCAGCTGGCATTGGTC |
| | TAGAGCTTTATAAGGCCATTCAGCGAGGGACCATCAAGTGCAACTTTGCGGGGGTTGCCTTGGGTGA |
| | TTCCTGGATCTCCCCTGTTGATTCGGTGCTCTCCTGGGGACCTTACCTGTACAGCATGTCTCTTCTC |
| | GAAGACAAAGGTCTGGCAGAGGTGTCTAAGGTTGCAGAGCAAGTACTGAATGCCGTAAATAAGGGGC |
| | TCTACAGAGAGGCCACAGAGCTGTGGGGGAAAGCAGAAATGATCATTGAACAGAACACAGATGGGGT |
| | GAACTTCTATAACATCTTAACTAAAAGCACTCCCACGTCTACAATGGAGTCGAGTCTAGAATTCACA |
| | CAGAGCCACCTAGTTTGTCTTTGTCAGCGCCACGTGAGACACCTACAACGAGATGCCTTAAGCCAGC |
| | TCATGAATGGCCCCATCAGAAAGAAGCTCAAAATTATTCCTGAGGATCAATCCTGGGGAGGCCAGGC |
| | TACCAACGTCTTTGTGAACATGGAGGAGGACTTCATGAAGCCAGTCATTAGCATTGTGGACGAGTTG |
| | CTGGAGGCAGGGATCAACGTGACGGTGTATAATGGACAGCTGGATCTCATCGTAGATACCATGGGTC |
| | AGGAGGCCTGGGTGCGGAAACTGAAGTGGCCAGAACTGCCTAAATTCAGTCAGCTGAAGTGGAAGGC |
| | CCTGTACAGTGACCCTAAATCTTTGGAAACATCTGCTTTTGTCAAGTCCTACAAGAACCTTGCTTTC |
| | TACTGGATTCTGAAAGCTGGTCATATGGTTCCTTCTGACCAAGGGGACATGGCTCTGAAGATGATGA |
| | GACTGGTGACTCAGCAAGAATAGC |

| | | |
|---|---|---|
| ORF Start: at 2 | | ORF Stop: TAG at 1361 |
| SEQ ID NO: 178 | 453 aa | MW at 50931.3kD |

| | |
|---|---|
| NOV23h, | TMELALRRSPVPRWLLLLPLLLGLNAGAVIDWPTEEGKEVWDYVTVRKDAYMFWWLYYATNSCKNFS |
| 171095500 | |
| Protein Sequence | ELPLVMWLQGGPGGSSTGFGNFEEIGPLDSDLKPRKTTWLQAASLLFVDNPVGTGFSYVNGSGAYAK |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLVCLCQRHVRHLQRDALSQLMNGPIRKKLKIIPEDQSWGGQA |
| | TNVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQE |

| | |
|---|---|
| SEQ ID NO: 179 | 1364 bp |

| | |
|---|---|
| NOV23i, | CACCATGGAGCTGGCACTGCGGCGCTCTCCCGTCCCGCGGTGGTTGCTGCTGCTGCCGCTGCTGCTG |
| 171095508 | |
| DNA Sequence | GGCCTGAACGCAGGAGCTGTCATTGACTGGCCCACAGAGGAGGGCAAGGAAGTATGGGATTATGTGA |
| | CGGTCCGCAAGGATGCCTACATGTTCTGGTGGCTCTATTATGCCACCAACTCCTGCAAGAACTTCTC |
| | AGAACTGCCCCTGGTCATGTGGCTTCAGGGCGGTCCAGGCGGTTCTAGCACTGGATTTGGAAACTTT |
| | GAGGAAATTGGGCCCCTTGACAGTGATCTCAAACCACGGAAAACCACCTGGCTCCAGGCTGCCAGTC |
| | TCCTATTTGTGGATAATCCCGTGGGCACTGGGTTCAGTTATGTGAATGGTAGTGGTGCCTATGCCAA |
| | GGACCTAGCTATGGTGGCTTCAGACATGATGGTTCTCCTGAAGACCTTCTTCAGTTGCCACAAAGAA |
| | TTCCAGACAGTTCCATTCTACATTTTCTCAGAGTCCTATGGAGGAAAAATGGCAGCTGGCATTGGTC |
| | TAGAGCTTTATAAGGCCATTCAGCGAGGGACCATCAAGTGCAACTTTGCGGGGGTTGCCTTGGGTGA |
| | TTCCTGGATCTCCCCTGTTGATTCGGTGCTCTCCTGGGGACCTTACCTGTACAGCATGTCTCTTCTC |
| | GAAGACAAAGGTCTGGCAGAGGTGTCTAAGGTTGCAGAGCAAGTACTGAATGCCGTAAATAAGGGGC |
| | TCTACAGAGAGGCCACAGAGCTGTGGGGGAAAGCAGAAATGATCATTGAACAGAACACAGATGGGGT |
| | GAACTTCTATAACATCTTAACTAAAAGCACTCCCACGTCTACAATGGAGTCGAGTCTAGAATTCACA |
| | CAGAGCCACCTAGTTTGTCTTTGTCAGCGCCACGTGAGACACCTACAACGAGATGCCTTAAGCCAGC |
| | TCATGAATGGCCCCATCAGAAAGAAGCTCAAAATTATTCCTGAGGATCAATCCTGGGGAGGCCAGGC |
| | TACCAACGTCTTTGTGAACATGGAGGAGGACTTCATGAAGCCAGTCATTAGCATTGTGGACGAGTTG |
| | CTGGAGGCAGGGATCAACGTGACGGTGTATAATGGACAGCTGGATCTCATCGTAGATACCATGGGTC |
| | AGGAGGCCTGGGTGCGGAAACTGAAGTGGCCAGAACTGCCTAAATTCAGTCAGCTGAAGTGGAAGGC |
| | CCTGTACAGTGACCCTAAATCTTTGGAAACATCTGCTTTTGTCAAGTCCTACAAGAACCTTGCTTTC |
| | TACTGGATTCTGAAAGCTGGTCATATGGTTCCTTCTGACCAAGGGGACATGGCTCTGAAGATGATGA |
| | GACTGGTGACTCAGCAAGAATAGC |

| | | |
|---|---|---|
| ORF Start: at 2 | | ORF Stop: TAG at 1361 |
| SEQ ID NO: 180 | 453 aa | MW at 50931.3kD |

| | |
|---|---|
| NOV23i | TMELALRRSPVPRWLLLLPLLLGLNAGAVIDWPTEEGKEVWDYVTVRKDAYMFWWLYYATNSCKNFS |
| 171095508 | |
| Protein | ELPLVMWLQGGPGGSSTGFGNFEEIGPLDSDLKPRKTTWLQAASLLFVDNPVGTGFSYVNGSGAYAK |
| Sequence | |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLVCLCQRHVRHLQRDALSQLMNGPIRKKLKIIPEDQSWGGQA |
| | TNVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQE |

| | |
|---|---|
| SEQ ID NO: 181 | 1364 bp |

| | |
|---|---|
| NOV23j, | CACCATGGAGCTGGCACTGCGGCGCTCTCCCGTCCCGCGGTGGTTGCTGCTGCTGCCGCTGCTGCTG |
| 171095572 | |
| DNA Sequence | GGCCTGAACGCAGGAGCTGTCATTGACTGGCCCACAGAGGAGGGCAAGGAAGTATGGGATTATGTGA |
| | CGGTCCGCAAGGATGCCTACATGTTCTGGTGGCTCTATTATGCCACCAACTCCTGCAAGAACTTCTC |
| | AGAACTGCCCCTGGTCATGTGGCTTCAGGGCGGTCCAGGCGGTTCTAGCACTGGATTTGGAAACTTT |
| | GAGGAAATTGGGCCCCTTGACAGTGATCTCAAACCACGGAAAACCACCTGGCTCCAGGCTGCCAGTC |
| | TCCTATTTGTGGATAATCCCGTGGGCACTGGGTTCAGTTATGTGAATGGTAGTGGTGCCTATGCCAA |
| | GGACCTAGCTATGGTGGCTTCAGACATGATGGTTCTCCTGAAGACCTTCTTCAGTTGCCACAAAGAA |
| | TTCCAGACAGTTCCATTCTACATTTTCTCAGAGTCCTATGGAGGAAAAATGGCAGCTGGCATTGGTC |
| | TAGAGCTTTATAAGGCCATTCAGCGAGGGACCATCAAGTGCAACTTTGCGGGGGTTGCCTTGGGTGA |
| | TTCCTGGATCTCCCCTGTTGATTCGGTGCTCTCCTGGGGACCTTACCTGTACAGCATGTCTCTTCTC |
| | GAAGACAAAGGTCTGGCAGAGGTGTCTAAGGTTGCAGAGCAAGTACTGAATGCCGTAAATAAGGGGC |
| | TCTACAGAGAGGCCACAGAGCTGTGGGGGAAAGCAGAAATGATCATTGAACAGAACACAGATGGGGT |
| | GAACTTCTATAACATCTTAACTAAAAGCACTCCCACGTCTACAATGGAGTCGAGTCTAGAATTCACA |
| | CAGAGCCACCTAGTTTGTCTTTGTCAGCGCCACGTGAGACACCTACAACGAGATGCCTTAAGCCAGC |
| | TCATGAATGGCCCCATCAGAAAGAAGCTCAAAATTATTCCTGAGGATCAATCCTGGGGAGGCCAGGC |
| | TACCAACGTCTTTGTGAACATGGAGGAGGACTTCATGAAGCCAGTCATTAGCATTGTGGACGAGTTG |
| | CTGGAGGCAGGGATCAACGTGACGGTGTATAATGGACAGCTGGATCTCATCGTAGATACCATGGGTC |
| | AGGAGGCCTGGGTGCGGAAACTGAAGTGGCCAGAACTGCCTAAATTCAGTCAGCTGAAGTGGAAGGC |
| | CCTGTACAGTGACCCTAAATCTTTGGAAACATCTGCTTTTGTCAAGTCCTACAAGAACCTTGCTTTC |
| | TACTGGATTCTGAAAGCTGGTCATATGGTTCCTTCTGACCAAGGGGACATGGCTCTGAAGATGATGA |
| | GACTGGTGACTCAGCAAGAATAGC |

| | | |
|---|---|---|
| ORF Start: at 2 | | ORF Stop: TAG at 1361 |
| SEQ ID NO: 182 | 453 aa | MW at 50931.3kD |

| | |
|---|---|
| NOV23j, | TMELALRRSPVPRWLLLLPLLLGLNAGAVIDWPTEEGKEVWDYVTVRKDAYMFWWLYYATNSCKNFS |
| 171095572 | |
| Protein | ELPLVMWLQGGPGGSSTGFGNFEEIGPLDSDLKPRKTTWLQAASLLFVDNPVGTGFSYVNGSGAYAK |
| Sequence | |
| | DLAMVASDMMVLLKTFFSCHKEFQTVPFYIFSESYGGKMAAGIGLELYKAIQRGTIKCNFAGVALGD |
| | SWISPVDSVLSWGPYLYSMSLLEDKGLAEVSKVAEQVLNAVNKGLYREATELWGKAEMIIEQNTDGV |
| | NFYNILTKSTPTSTMESSLEFTQSHLVCLCQRHVRHLQRDALSQLMNGPIRKKLKIIPEDQSWGGQA |
| | TNVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQE |

| | |
|---|---|
| SEQ ID NO: 183 | 1386 bp |

| | |
|---|---|
| NOV23k, | CCACCATGGGCCACCATCACCACCATCACGAGCTGGCACTGCGGCGCTCTCCCGTCCCGCGGTGGTT |
| 171095162 DNA | |
| Sequence | GCTGCTGCTGCCGCTGCTGCTGGGCCTGAACGCAGGAGCTGTCATTGACTGGCCCACAGAGGAGGGC |
| | AAGGAAGTATGGGATTATGTGACGGTCCGCAAGGATGCCTACATGTTCTGGTGGCTCTATTATGCCA |
| | CCAACTCCTGCAAGAACTTCTCAGAACTGCCCCTGGTCATGTGGCTTCAGGGCGGTCCAGGCGGTTC |
| | TAGCACTGGATTTGGAAACTTTGAGGAAATTGGGCCCCTTGACAGTGATCTCAAACCACGGAAAACC |
| | ACCTGGCTCCAGGCTGCCAGTCTCCTATTTGTGGATAATCCCGTGGGCACTGGGTTCAGTTATGTGA |
| | ATGGTAGTGGTGCCTATGCCAAGGACCTGGCTATGGTGGCTTCAGACATGATGGTTCTCCTGAAGAC |
| | CTTCTTCAGTTGCCACAAAGAATTCCAGACAGTTCCATTCTACATTTTCTCAGAGTCCTATGGAGGA |
| | AAAATGGCAGCTGGCATTGGTCTAGAGCTTTATAAGGCCATTCAGCGAGGGACCATCAAGTGCAACT |
| | TTGCGGGGGTTGCCTTGGGTGATTCCTGGATCTCCCCTGTTGATTCGGTGCTCTCCTGGGGACCTTA |
| | CCTGTACAGCATGTCTCTTCTCGAAGACAAAGGTCTGGCAGAGGTGTCTAAGGTTGCAGAGCAAGTA |
| | CTGAATGCCGTAAATAAGGGGCTCTACAGAGAGGCCACAGAGCTGTGGGGGAAAGCAGAAATGATCA |
| | TTGAACAGAACACAGATGGGGTGAACTTCTATAACATCTTAACTAAAAGCACTCCCACGTCTACAAT |
| | GGAGTCGAGTCTAGAATTCACACAGAGCCACCTAGTTTGTCTTTGTCAGCGCCACGTGAGACACCTA |
| | CAACGAGATGCCTTAAGCCAGCTCATGAATGGCCCCATCAGAAAGAAGCTCAAAATTATTCCTGAGG |
| | ATCAATCCTGGGGAGGCCAGGCTACCAACGTCTTTGTGAACATGGAGGAGGACTTCATGAAGCCAGT |
| | CATTAGCATTGTGGACGAGTTGCTGGAGGCAGGGATCAACGTGACGGTGTATAATGGACAGCTGGAT |
| | CTCATCGTAGATACCATGGGTCAGGAGGCCTGGGTGCGGAAACTGAAGTGGCCAGAACTGCCTAAAT |
| | TCAGTCAGCTGAAGTGGAAGGCCCTGTACAGTGACCCTAAATCTTTGGAAACATCTGCTTTTGTCAA |
| | GTCCTACAAGAACCTTGCTTTCTACTGGATTCTGAAAGCTGGTCATATGGTTCCTTCTGACCAAGGG |
| | GACATGGCTCTGAAGATGATGAGACTGGTGACTCAGCAAGAATAGC |

| | | |
|---|---|---|
| ORF Start: at 3 | | ORF Stop: TAG at 1383 |
| SEQ ID NO: 184 | 460 aa | MW at 51811.2 kD |

| | |
|---|---|
| NOV23k, | TMGHHHHHHELALRRSPVPRWLLLLPLLLGLNAGAVIDWPTEEGKEVWDYVTVRKDAYMFWWLYYAT |
| 171095162 | |
| Protein Sequence | NSCKNFSELPLVMWLQGGPGGSSTGFGNFEEIGPLDSDLKPRKTTWLQAASLLFVDNPVGTGFSYVN |
| | GSGAYAKDLAMVASDMMVLLKTFFSCHKEFQTVPFYIFSESYGGKMAAGIGLELYKAIQRGTIKCNF |
| | AGVALGDSWISPVDSVLSWGPYLYSMSLLEDKGLAEVSKVAEQVLNAVNKGLYREATELWGKAEMII |
| | EQNTDGVNFYNILTKSTPTSTMESSLEFTQSHLVCLCQRHVRHLQRDALSQLMNGPIRKKLKIIPED |
| | QSWGGQATNVFVNMEEDFMKPVISIVDELLEAGINVTVYNGQLDLIVDTMGQEAWVRKLKWPELPKF |
| | SQLKWKALYSDPKSLETSAFVKSYKNLAFYWILKAGHMVPSDQGDMALKMMRLVTQQE |

| | |
|---|---|
| SEQ ID NO: 185 | 1394 bp |

| | |
|---|---|
| N0V23l, | CCACCATGGGCCACCATCACCACCATCACGAGCTGGCACTGCGGCGCTCTCCCGTCCCGCGGTGGTT |
| 171095169 | |
| DNA Sequence | GCTGCTGCTGCCGCTGCTGCTGGGCCTGAACGCAGGAGCTGTCATTGACTGGCCCACAGAGGAGGGC |
| | AAGGAAGTATGGGATTATGTGACGGTCCGCAAGGATGCCTACATGTTCTGGTGGCTCTATTATGCCA |
| | CCAACTCCTGCAAGAACTTCTCAGAACTGCCCCTGGTCATGTGGCTTCAGGGCGGTCCAGGCGGTTC |
| | TAGCACTGGATTTGGAAACTTTGAGGAAATTGGGCCCCTTGACAGTGATCTCAAACCACGGAAAACC |
| | ACCTGGCTCCAGGCTGCCAGTCTCCTATTTGTGGATAATCCCGTGGGCACTGGGTTCAGTTATGTGA |
| | ATGGTAGTGGTGCCTATGCCAAGGACCTGGCTATGGTGGCTTCAGACATGATGGTTCTCCTGAAGAC |
| | CTTCTTCAGTTGCCACAAAGAATTCCAGACAGTTCCATTCTACATTTTCTCAGAGTCCTATGGAGGA |
| | AAAATGGCAGCTGGCATTGGTCTAGAGCTTTATAAGGCCATTCAGCGAGGGACCATCAAGTGCAACT |
| | TTGCGGGGGTTGCCTTGGGTGATTCCTGGATCTCCCCTGTTGATTCGGTGCTCTCCTGGGGACCTTA |
| | CCTGTACAGCATGTCTCTTCTCGAAGACAAAGGTCTGGCAGAGGTGTCTAAGGTTGCAGAGCAAGTA |
| | CTGAATGCCGTAAATAAGGGGCTCTACAGAGAGGCCACAGAGCTGTGGGGGAAAGCAGAAATGATCA |
| | TTGAACAGAACACAGATGGGGTGAACTTCTATAACATCTTAACTAAAAGCACTCCCACGTCTACAAT |
| | GGAGTCGAGTCTAGAATTCACACAGAGCCACCTAGTTTGTCTTTGTCAGCGCCACGTGAGACACCTA |
| | CAACGAGATGCCTTAAGCCAGCTCATGAATGGCCCCATCAGAAAGAAGCTCAAAATTATTCCTGAGG |
| | ATCAATCCTGGGGAGGCCAGGCTACCAACGTCTTTGTGAACATGGAGGAGGACTTCATGAAGCCAGT |
| | CATTAGCATTGTGGACGAGTTGCTGGAGGCAGGGATCAACGTGACGGTGTATAATGGACAGCTGGAT |
| | CTCATCGTAGATACCATGGGTCAGGAGGCCTGGGTGCGGAAACTGAAGTGGCCAGAACTGCCTAAAT |
| | TCAGTCAGCTGAAGTGGAAGGCCCTGTACAGTGACCCTAAATCTTTGGAAACATCTGCTTTTGTCAA |
| | GTCCTACAAGAACCTTGCTTTCTACTGGATTCTGAAAGCTGGTCATATGGTTCCTTCTGACCAAGGG |
| | GACATGGCTCTGAAGATGATGAGACTGGTGACTCAGCAAGAATAGCGCGGCCGC |

| | | |
|---|---|---|
| ORF Start: at 3 | | ORF Stop: TAG at 1383 |
| SEQ ID NO: 186 | 460 aa | MW at 51811.2 kD |

| | |
|---|---|
| N0V23l, | TMGHHHHHHELALRRSPVPRWLLLLPLLLGLNAGAVIDWPTEEGKEVWDYVTVRKDAYMFWWLYYAT |
| 171095169 | |
| Protein | NSCKNFSELPLVMWLQGGPGGSSTGFGNFEEIGPLDSDLKPRKTTWLQAASLLFVDNPVGTGFSYVN |
| Sequence | |
| | GSGAYAKDLAMVASDMMVLLKTFFSCHKEFQTVPFYIFSESYGGKMAAGIGLELYKAIQRGTIKCNF |
| | AGVALGDSWISPVDSVLSWGPYLYSMSLLEDKGLAEVSKVAEQVLNAVNKGLYREATELWGKAEMII |
| | EQNTDGVNFYNILTKSTPTSTMESSLEFTQSHLVCLCQRHVRHLQRDALSQLMNGPIRKKLKIIPED |
| | QSWGGQATNVFVNMEEDFMKPVISIVDELLEAGINVTVYNGQLDLIVDTMGQEAWVRKLKWPELPKF |
| | SQLKWKALYSDPKSLETSAFVKSYKNLAFYWILKAGHMVPSDQGDMALKMMRLVTQQE |

| | |
|---|---|
| SEQ ID NO: 187 | 1344 bp |

| | |
|---|---|
| NOV23m, | ACCATGGTAAGCGCTATTGTTTTATATGTGCTTTTGGCGGCGGCGGCGCATTCTGCCTTTGCGGCT |
| 222681273 DNA | |
| Sequence | GTCATTGACTGGCCCACAGAGGAGGGCAAGGAAGTATGGGATTATGTGACGGTCCGCAAGGATGCC |
| | TACATGTTCTGGTGGCTCTATTATGCCACCAACTCCTGCAAGAACTTCTCAGAACTGCCCCTGGTC |
| | ATGTGGCTTCAGGGCGGTCCAGGCGGTTCTAGCACTGGATTTGGAAACTTTGAGGAAATTGGGCCC |
| | CTTGACAGTGATCTCAAACCACGGAAAACCACCTGGCTCCAGGCTGCCAGTCTCCTATTTGTGGAT |
| | AATCCCGTGGGCACTGGGTTCAGTTATGTGAATGGTAGTGGTGCCTATGCCAAGGACCTAGCTATG |
| | GTGGCTTCAGACATGATGGTTCTCCTGAAGACCTTCTTCAGTTGCCACAAAGAATTCCAGACAGTT |
| | CCATTCTACATTTTCTCAGAGTCCTATGGAGGAAAAATGGCAGCTGGCATTGGTCTAGAGCTTTAT |
| | AAGGCCATTCAGCGAGGGACCATCAAGTGCAACTTTGCGGGGGTTGCCTTGGGTGATTCCTGGATC |
| | TCCCCTGTTGATTCGGTGCTCTCCTGGGGACCTTACCTGTACAGCATGTCTCTTCTCGAAGACAAA |
| | GGTCTGGCAGAGGTGTCTAAGGTTGCAGAGCAAGTACTGAATGCCGTAAATAAGGGGCTCTACAGA |
| | GAGGCCACAGAGCTGTGGGGGAAAGCAGAAATGATCATTGAACAGAACACAGATGGGGTGAACTTC |
| | TATAACATCTTAACTAAAAGCACTCCCACGTCTACAATGGAGTCGAGTCTAGAATTCACACAGAGC |
| | CACCTAGTTTGTCTTTGTCAGCGCCACGTGAGACACCTACAACGAGATGCCTTAAGCCAGCTCATG |
| | AATGGCCCCATCAGAAAGAAGCTCAAAATTATTCCTGAGGATCAATCCTGGGGAGGCCAGGCTACC |
| | AACGTCTTTGTGAACATGGAGGAGGACTTCATGAAGCCAGTCATTAGCATTGTGGACGAGTTGCTG |
| | GAGGCAGGGATCAACGTGACGGTGTATAATGGACAGCTGGATCTCATCGTAGATACCATGGGTCAG |
| | GAGGCCTGGGTGCGGAAACTGAAGTGGCCAGAACTGCCTAAATTCAGTCAGCTGAAGTGGAAGGCC |
| | CTGTACAGTGACCCTAAATCTTTGGAAACATCTGCTTTTGTCAAGTCCTACAAGAACCTTGCTTTC |
| | TACTGGATTCTGAAAGCTGGTCATATGGTTCCTTCTGACCAAGGGGACATGGCTCTGAAGATGATG |
| | AGACTGGTGACTCAGCAAGAATAG |

| | | |
|---|---|---|
| ORF Start: at 1 | | ORF Stop: TAG at 1342 |
| SEQ ID NO: 188 | 447 aa | MW at 50037.2 kD |

| | |
|---|---|
| NOV23m, | TMVSAIVLYVLLAAAAHSAFAAVIDWPTEEGKEVWDYVTVRKDAYMFWWLYYATNSCKNFSELPLV |
| 222681273 | |
| Protein Sequence | MWLQGGPGGSSTGFGNFEEIGPLDSDLKPRKTTWLQAASLLFVDNPVGTGFSYVNGSGAYAKDLAM |
| | VASDMMVLLKTFFSCHKEFQTVPFYIFSESYGGKMAAGIGLELYKAIQRGTIKCNFAGVALGDSWT |
| | SPVDSVLSWGPYLYSMSLLEDKGLAEVSKVAEQVLNAVNKGLYREATELWGKAEMIIEQNTDGVNF |
| | YNILTKSTPTSTMESSLEFTQSHLVCLCQRHVRHLQRDALSQLMNGPIRKKLKIIPEDQSWGGQAT |
| | NVFVNMEEDFMKPVISIVDELLEAGINVTVYNGQLDLIVDTMGQEAWVRKLKWPELPKFSQLKWKA |
| | LYSDPKSLETSAFVKSYKNLAFYWILKAGHMVPSDQGDMALKMMRLVTQQE |

| | |
|---|---|
| SEQ ID NO: 189 | 1362 bp |

| | |
|---|---|
| NOV23n, | ACCATGGTAAGCGCTATTGTTTTATATGTGCTTTTCGCGGCGGCGGCGCATTCTGCCTTTGCGGCTG |
| 201536204 DNA | |
| Sequence | TCATTGACTCGCCCACAGAGGAGGGCAAGGAAGTATGGGATTATGTGACGGTCCGCAAGGATGCCTA |
| | CATGTTCTGGTGGCTCTATTATGCCACCAACTCCTGCAAGAACTTCTCAGAACTGCCCCTGGTCATG |
| | TGGCTTCAGGGCGGTCCAGGCGGTTCTAGCACTGGATTTGGAAACTTTGAGGAAATTGGGCCCCTTG |
| | ACAGTGATCTCAAACCACGGAAAACCACCTGGCTCCAGGCTGCCAGTCTCCTATTTGTGGATAATCC |
| | CGTGGGCACTGGGTTCAGTTATGTGAATGGTAGTGGTGCCTATGCCAAGGACCTAGCTATGGTGGCT |
| | TCAGACATGATGGTTCTCCTGAAGACCTTCTTCAGTTGCCACAAAGAATTCCAGACAGTTCCATTCT |
| | ACATTTTCTCAGAGTCCTATGGAGGAAAAATGGCAGCTGGCATTGGTCTAGAGCTTTATAAGGCCAT |
| | TCAGCGAGGGACCATCAAGTGCAACTTTGCGGGGGTTGCCTTGGGTGATTCCTGGATCTCCCCTGTT |
| | GATTCGGTGCTCTCCTGGGGACCTTACCTGTACAGCATGTCTCTTCTCGAAGACAAAGGTCTGGCAG |
| | AGGTGTCTAAGGTTGCAGAGCAAGTACTGAATGCCGTAAATAAGGGGCTCTACAGAGAGGCCACAGA |
| | GCTGTGGGGGAAAGCAGAAATGATCATTGAACAGAACACAGATGGGGTGAACTTCTATAACATCTTA |
| | ACTAAAAGCACTCCCACGTCTACAATGGAGTCGAGTCTAGAATTCACACAGAGCCACCTAGTTTGTC |
| | TTTGTCAGCGCCACGTGAGACACCTACAACGAGATGCCTTAAGCCAGCTCATGAATGGCCCCATCAG |
| | AAAGAAGCTCAAAATTATTCCTGAGGATCAATCCTGGGGAGGCCAGGCTACCAACGTCTTTGTGAAC |
| | ATGGAGGAGGACTTCATGAAGCCAGTCATTAGCATTGTGGACGAGTTGCTGGAGGCAGGGATCAACG |
| | TGACGGTGTATAATGGACAGCTGGATCTCATCGTAGATACCATGGGTCAGGAGGCCTGGGTGCGGAA |
| | ACTGAAGTGGCCAGAACTGCCTAAATTCAGTCAGCTGAAGTGGAAGGCCCTGTACAGTGACCCTAAA |
| | TCTTTGGAAACATCTGCTTTTGTCAAGTCCTACAAGAACCTTGCTTTCTACTGGATTCTGAAAGCTG |
| | GTCATATGGTTCCTTCTGACCAAGGGGACATGGCTCTGAAGATGATGAGACTGGTGACTCAGCAAGA |
| | ACACCATCACCACCATCACTAG |

| | | |
|---|---|---|
| ORF Start: at 1 | | ORF Stop: TAG at 1360 |
| SEQ ID NO: 190 | 453 aa | MW at 50860.0kD |

| | |
|---|---|
| NOV23n, | TMVSAIVLYVLLAAAAHSAFAAVIDWPTEEGKEVWDYVTVRKDAYMFWWLYYATNSCKNFSELPLVM |
| 201536204 | |
| Protein Sequence | WLQGGPGGSSTGFGNFEEIGPLDSDLKPRKTTWLQAASLLFVDNPVGTGFSYVNGSGAYAKDLAMVA |
| 0 | |
| | SDMMVLLKTFFSCHKEFQTVPFYIFSESYGGKMAAGIGLELYKAIQRGTIKCNFAGVALGDSWISPV |
| 0 | |
| | DSVLSWGPYLYSMSLLEDKGLAEVSKVAEQVLNAVNKGLYREATELWGKAEMIIEQNTDGVNFYNIL |
| 0 | |
| | TKSTPTSTMESSLEFTQSHLVCLCQRHVRHLQRDALSQLMNGPIRKKLKIIPEDQSWGGQATNVFVN |
| 0 | |
| | MEEDFMKPVISIVDELLEAGINVTVYNGQLDLIVDTMGQEAWVRKLKWPELPKFSQLKWKALYSDPK |
| 0 | |
| | SLETSAFVKSYKNLAFYWILKAGHMVPSDQGDMALKMMRLVTQQEHHHHHH |
| 0 | |

| | |
|---|---|
| SEQ ID NO: 191 | 1278 bp |

| | |
|---|---|
| NOV23o, | GCTGTCATTGACTGGCCCACAGAGGAGGGCAAGGAAGTATGGGATTATGTGACGGTCCGCAAGGATG |
| CG55078-02 | |
| DNA Sequence | CCTACATGTTCTGGTGGCTCTATTATGCCACCAACTCCTGCAAGAACTTCTCAGAACTGCCCCTGGT |
| | CATGTGGCTTCAGGGCGGTCCAGGCGGTTCTAGCACTGGATTTGGAAACTTTGAGGAAATTGGGCCC |
| | CTTGACAGTGATCTCAAACCACGGAAAACCACCTGGCTCCAGGCTGCCAGTCTCCTATTTGTGGATA |
| | ATCCCGTGGGCACTGGGTTCAGTTATGTGAATGGTAGTGGTGCCTATGCCAAGGACCTGGCTATGGT |
| | GGCTTCAGACATGATGGTTCTCCTGAAGACCTTCTTCAGTTGCCACAAAGAATTCCAGACAGTTCCA |
| | TTCTACATTTTCTCAGAGTCCTATGGAGGAAAAATGGCAGCTGGCATTGGTCTAGAGCTTTATAAGG |
| | CCATTCAGCGAGGGACCATCAAGTGCAACTTTGCGGGGGTTGCCTTGGGTGATTCCTGGATCTCCCC |
| | TGTTGATTCGGTGCTCTCCTGGGGACCTTACCTGTACAGCATGTCTCTTCTCGAAGACAAAGGTCTG |
| | GCAGAGGTGTCTAAGGTTGCAGAGCAAGTACTGAATGCCGTAAATAAGGGGCTCTACAGAGAGGCCA |
| | CAGAGCTGTGGGGGAAAGCAGAAATGATCATTGAACAGAACACAGATGGGGTGAACTTCTATAACAT |
| | CTTAACTAAAAGCACTCCCACGTCTACAATGGAGTCGAGTCTAGAATTCACACAGAGCCACCTAGTT |
| | TGTCTTTGTCAGCGCCACGTGAGACACCTACAACGAGATGCCTTAAGCCAGCTCATGAATGGCCCCA |
| | TCAGAAAGAAGCTCAAAATTATTCCTGAGGATCAATCCTGGGGAGGCCAGGCTACCAACGTCTTTGT |
| | GAACATGGAGGAGGACTTCATGAAGCCAGTCATTAGCATTGTGGACGAGTTGCTGGAGGCAGGGATC |
| | AACGTGACGGTGTATAATGGACAGCTGGATCTCATCGTAGATACCATGGGTCAGGAGGCCTGGGTGC |
| | GGAAACTGAAGTGGCCAGAACTGCCTAAATTCAGTCAGCTGAAGTGGAAGGCCCTGTACAGTGACCC |
| | TAAATCTTTGGAAACATCTGCTTTTGTCAAGTCCTACAAGAACCTTGCTTTCTACTGGATTCTGAAA |
| | GCTGGTCATATGGTTCCTTCTGACCAAGGGGACATGGCTCTGAAGATGATGAGACTGGTGACTCAGC |
| | AAGAA |

| | | |
|---|---|---|
| ORF Start: at 1 | | ORF Stop: end of sequence |
| SEQ ID NO: 192 | 426 aa | MW at 47935.7kD |

| | |
|---|---|
| NOV23o, | AVIDWPTEEGKEVWDYVTVRKDAYMFWWLYYATNSCKNFSELPLVMWLQGGPGGSSTGFGNFEEIGP |
| CG55078-02 | |
| Protein Sequence | LDSDLKPRKTTWLQAASLLFVDNPVGTGESYVNGSGAYAKDLAMVASDMMVLLKTFFSCHKEFQTVP |
| | FYIFSESYGGKMAAGIGLELYKAIQRGTIKCNFAGVALGDSWISPVDSVLSWGPYLYSMSLLEDKGL |
| | AEVSKVAEQVLNAVNKGLYREATELWGKAEMIIEQNTDGVNFYNILTKSTPTSTMESSLEFTQSHLV |
| | CLCQRHVRHLQRDALSQLMNGPIRKKLKIIPEDQSWGGQATNVFVNMEEDFMKPVISIVDELLEAGI |
| | NVTVYNGQLDLIVDTMGQEAWVRKLKWPELPKFSQLKWKALYSDPKSLETSAFVKSYKNLAFYWILK |
| | AGHMVPSDQGDMALKMMRLVTQQE |

| | |
|---|---|
| SEQ ID NO: 193 | 1719 bp |

| | |
|---|---|
| NOV23p, | TAACACTCCGCCCCATTGACGCAAATGGGCGGTAGGCGTGTACGGTGGGAGGTCTATATAAGCAGAG |
| CG55078-05 | |
| DNA Sequence | CTCTCTGGCTAACTAGAGAACCCACTGCTTACTGGCTTATCGAAATTAATACGACTCACTATAGGGA |
| | GACCCAAGCTGGCTAGCGTTTAAACTTAAGCTTGGTACCGAGCTCGGATCCCCACCATGGAGCTGGC |
| | ACTGCGGCGCTCTCCCGTCCCGCGGTGGTTGCTGCTGCTGCCGCTGCTGCTGGGCCTGAACGCAGGA |
| | GCTGTCATTGACTGGCCCACAGAGGAGGGCAAGGAAGTATGGGATTATGTGACGGTCCGCAAGGATG |
| | CCTACATGTTCTGGTGGCTCTATTATGCCACCAACTCCTGCAAGAACTTCTCAGAACTGCCCCTGGT |
| | CATGTGGCTTCAGGGCGGTCCAGGCGGTTCTAGCACTGGATTTGGAAACTTTGAGGAAATTGGGCCC |
| | CTTGACAGTGATCTCAAACCACGGAAAACCACCTGGCTCCAGGCTGCCAGTCTCCTATTTGTGGATA |
| | ATCCCGTGGGCACTGGGTTCAGTTATGTGAATGGTAGTGGTGCCTATGCCAAGGACCTGGCTATGGT |
| | GGCTTCAGACATGATGGTTCTCCTGAAGACCTTCTTCAGTTGCCACAAAGAATTCCAGACAGTTCCA |
| | TTCTACATTTTCTCAGAGTCCTATGGAGGAAAAATGGCAGCTGGCATTGGTCTAGAGCTTTATAAGG |
| | CCATTCAGCGAGGGACCATCAAGTGCAACTTTGCGGGGGTTGCCTTGGGTGATTCCTGGATCTCCCC |
| | TGTTGATTCGGTGCTCTCCTGGGGACCTTACCTGTACAGCATGTCTCTTCTCGAAGACAAAGGTCTG |
| | GCAGAGGTGTCTAAGGTTGCAGAGCAAGTACTGAATGCCGTAAATAAGGGGCTCTACAGAGAGGCCA |
| | CAGAGCTGTGGGGGAAAGCAGAAATGATCATTGAACAGAACACAGATGGGGTGAACTTCTATAACAT |
| | CTTAACTAAAAGCACTCCCACGTCTACAATGGAGTCGAGTCTAGAATTCACACAGAGCCACCTAGTT |
| | TGTCTTTGTCAGCGCCACGTGAGACACCTACAACGAGATGCCTTAAGCCAGCTCATGAATGGCCCCA |
| | TCAGAAAGAAGCTCAAAATTATTCCTGAGGATCAATCCTGGGGAGGCCAGGCTACCAACGTCTTTGT |
| | GAACATGGAGGAGGACTTCATGAAGCCAGTCATTAGCATTGTGGACGAGTTGCTGGAGGCAGGGATC |
| | AACGTGACGGTGTATAATGGACAGCTGGATCTCATCGTAGATACCATGGGTCAGGAGGCCTGGGTGC |
| | GGAAACTGAAGTGGCCAGAACTGCCTAAATTCAGTCAGCTGAAGTGGAAGGCCCTGTACAGTGACCC |
| | TAAATCTTTGGAAACATCTGCTTTTGTCAAGTCCTACAAGAACCTTGCTTTCTACTGGATTCTGAAA |
| | GCTGGTCATATGGTTCCTTCTGACCAAGGGGACATGGCTCTGAAGATGATGAGACTGGTGACTCAGC |
| | AAGAACACCATCACCACCATCACTAGGCGGCCGCTCGAGTCTAGAGGGCCCGTCTAAACCCGCTGAT |
| | CAGCCTCGACTGTGCCTTCTAGTTGCCAGCCATCTGTTGTTTGCCCCTCCCCCGTGCCTTCCTTGAC |
| | CTGGAAGGTGCCACTCCCACTGTCCTTTCTAATAAAATGAGGAA |

| | | |
|---|---|---|
| ORF Start: ATG at 191 | | ORF Stop: at 1547 |
| SEQ ID NO: 194 | 452 aa | MW at 50830.2kD |

| | |
|---|---|
| NOV23p, | MELALRRSPVPRWLLLLPLLLGLNAGAVIDWPTEEGKEVWDYVTVRKDAYMFWWLYYATNSCKNFSE |
| CG55078-05 | |
| Protein Sequence | LPLVMWLQGGPGGSSTGFGNFEEIGPLDSDLKPRKTTWLQAASLLFVDNPVGTGFSYVNGSGAYAKD |
| | LAMVASDMMVLLKTFFSCHKEFQTVPFYIFSESYGGKMAAGIGLELYKAIQRGTIKCNFAGVALGDS |
| | WISPVDSVLSWGPYLYSMSLLEDKGLAEVSKVAEQVLNAVNKGLYREATELWGKAEMIIEQNTDGVN |
| | FYNILTKSTPTSTMESSLEFTQSHLVCLCQRHVRHLQRDALSQLMNGPIRKKLKIIPEDQSWGGQAT |
| | NVFVNMEEDFMKPVISIVDELLEAGINVTVYNGQLDLIVDTMGQEAWVRKLKWPELPKFSQLKWKAL |
| | YSDPKSLETSAFVKSYKNLAFYWILKAGHMVPSDQGDMALKMMRLVTQQE |

| | |
|---|---|
| SEQ ID NO: 195 | 1344 bp |

| | |
|---|---|
| NOV23q, | ACCATGGTAAGCGCTATTGTTTTATATGTGCTTTTGGCGGCGGCGGCGCATTCTGCCTTTGCGGCTG |
| CG55078-06 | |
| DNA Sequence | TCATTGACTGGCCCACAGAGGAGGGCAAGGAAGTATGGGATTATGTGACGGTCCGCAAGGATGCCTA |
| | CATGTTCTGGTGGCTCTATTATGCCACCAACTCCTGCAAGAACTTCTCAGAACTGCCCCTGGTCATG |
| | TGGCTTCAGGGCGGTCCAGGCGGTTCTAGCACTGGATTTGGAAACTTTGAGGAAATTGGGCCCCTTG |
| | ACAGTGATCTCAAACCACGGAAAACCACCTGGCTCCAGGCTGCCAGTCTCCTATTTGTGGATAATCC |
| | CGTGGGCACTGGGTTCAGTTATGTGAATGGTAGTGGTGCCTATGCCAAGGACCTAGCTATGGTGGCT |
| | TCAGACATGATGGTTCTCCTGAAGACCTTCTTCAGTTGCCACAAAGAATTCCAGACAGTTCCATTCT |
| | ACATTTTCTCAGAGTCCTATGGAGGAAAAATGGCAGCTGGCATTGGTCTAGAGCTTTATAAGGCCAT |
| | TCAGCGAGGGACCATCAAGTGCAACTTTGCGGGGGTTGCCTTGGGTGATTCCTGGATCTCCCCTGTT |
| | GATTCGGTGCTCTCCTGGGGACCTTACCTGTACAGCATGTCTCTTCTCGAAGACAAAGGTCTGGCAG |
| | AGGTGTCTAAGGTTGCAGAGCAAGTACTGAATGCCGTAAATAAGGGGCTCTACAGAGAGGCCACAGA |
| | GCTGTGGGGGAAAGCAGAAATGATCATTGAACAGAACACAGATGGGGTGAACTTCTATAACATCTTA |
| | ACTAAAAGCACTCCCACGTCTACAATGGAGTCGAGTCTAGAATTCACACAGAGCCACCTAGTTTGTC |
| | TTTGTCAGCGCCACGTGAGACACCTACAACGAGATGCCTTAAGCCAGCTCATGAATGGCCCCATCAG |
| | AAAGAAGCTCAAAATTATTCCTGAGGATCAATCCTGGGGAGGCCAGGCTACCAACGTCTTTGTGAAC |
| | ATGGAGGAGGACTTCATGAAGCCAGTCATTAGCATTGTGGACGAGTTGCTGGAGGCAGGGATCAACG |
| | TGACGGTGTATAATGGACAGCTGGATCTCATCGTAGATACCATGGGTCAGGAGGCCTGGGTGCGGAA |
| | ACTGAAGTGGCCAGAACTGCCTAAATTCAGTCAGCTGAAGTGGAAGGCCCTGTACAGTGACCCTAAA |
| | TCTTTGGAAACATCTGCTTTTGTCAAGTCCTACAAGAACCTTGCTTTCTACTGGATTCTGAAAGCTG |
| | GTCATATGGTTCCTTCTGACCAAGGGGACATGGCTCTGAAGATGATGAGACTGGTGACTCAGCAAGA |
| | ATAG |

| | | |
|---|---|---|
| ORF Start: at 1 | | ORF Stop: TAG at 1342 |
| SEQ ID NO: 196 | 447 aa | MW at 50037.2kD |

| | |
|---|---|
| NOV23q, | TMVSAIVLYVLLAAAAHSAFAAVIDWPTEEGKEVWDYVTVRKDAYMFWWLYYATNSCKNFSELPLVM |
| CG55078-06 | |
| Protein Sequence | WLQGGPGGSSTGFGNFEEIGPLDSDLKPRKTTWLQAASLLFVDNPVGTGFSYVNGSGAYAKDLAMVA |
| | SDMMVLLKTFFSCHKEFQTVPFYIFSESYGGKMAAGIGLELYKAIQRGTIKCNFAGVALGDSWISPV |
| | DSVLSWGPYLYSMSLLEDKGLAEVSKVAEQVLNAVNKGLYREATELWGKAEMIIEQNTDGVNFYNIL |
| | TKSTPTSTMESSLEFTQSHLVCLCQRHVRHLQRDALSQLMNGPIRKKLKIIPEDQSWGGQATNVFVN |
| | MEEDFMKPVISIVDELLEAGINVTVYNGQLDLIVDTMGQEAWVRKLKWPELPKFSQLKWKALYSDPK |
| | SLETSAFVKSYKNLAFYWILKAGHMVPSDQGDMALKMMRLVTQQE |

| | |
|---|---|
| SEQ ID NO: 197 | 1362 bp |

| | |
|---|---|
| NOV23r, | ACCATGGTAAGCGCTATTGTTTTATATGTGCTTTTGGCGGCGGCGGCGCATTCTGCCTTTGCGGCTG |
| CG55078-07 | |
| DNA Sequence | TCATTGACTGGCCCACAGAGGAGGGCAAGGAAGTATGGGATTATGTGACGGTCCGCAAGGATGCCTA |
| | CATGTTCTGGTGGCTCTATTATGCCACCAACTCCTGCAAGAACTTCTCAGAACTGCCCCTGGTCATG |
| | TGGCTTCAGGGCGGTCCAGGCGGTTCTAGCACTGGATTTGGAAACTTTGAGGAAATTGGGCCCCTTG |
| | ACAGTGATCTCAAACCACGGAAAACCACCTGGCTCCAGGCTGCCAGTCTCCTATTTGTGGATAATCC |
| | CGTGGGCACTGGGTTCAGTTATGTGAATGGTAGTGGTGCCTATGCCAAGGACCTAGCTATGGTGGCT |
| | TCAGACATGATGGTTCTCCTGAAGACCTTCTTCAGTTGCCACAAAGAATTCCAGACAGTTCCATTCT |
| | ACATTTTCTCAGAGTCCTATGGAGGAAAAATGGCAGCTGGCATTGGTCTAGAGCTTTATAAGGCCAT |
| | TCAGCGAGGGACCATCAAGTGCAACTTTGCGGGGGTTGCCTTGGGTGATTCCTGGATCTCCCCTGTT |
| | GATTCGGTCCTCTCCTGGGGACCTTACCTGTACAGCATGTCTCTTCTCGAAGACAAAGGTCTGGCAG |
| | AGGTGTCTAAGGTTGCAGAGCAAGTACTGAATGCCGTAAATAAGGGGCTCTACAGAGAGGCCACAGA |
| | GCTGTGGGGGAAAGCAGAAATGATCATTGAACAGAACACAGATGGGGTGAACTTCTATAACATCTTA |
| | ACTAAAAGCACTCCCACGTCTACAATGGAGTCGAGTCTAGAATTCACACAGAGCCACCTAGTTTGTC |
| | TTTGTCAGCGCCACGTGAGACACCTACAACGAGATGCCTTAAGCCAGCTCATGAATGGCCCCATCAG |
| | AAAGAAGCTCAAAATTATTCCTGAGGATCAATCCTGGGGAGGCCAGGCTACCAACGTCTTTGTGAAC |
| | ATGGAGGAGGACTTCATGAAGCCAGTCATTAGCATTGTGGACGAGTTGCTGGAGGCAGGGATCAACG |
| | TGACGGTGTATAATGGACAGCTGGATCTCATCGTAGATACCATGGGTCAGGAGGCCTGGGTGCGGAA |
| | ACTGAAGTGGCCAGAACTGCCTAAATTCAGTCAGCTGAAGTGGAAGGCCCTGTACAGTGACCCTAAA |
| | TCTTTGGAAACATCTGCTTTTGTCAAGTCCTACAAGAACCTTGCTTTCTACTGGATTCTGAAAGCTG |
| | GTCATATGGTTCCTTCTGACCAAGGGGACATGGCTCTGAAGATGATGAGACTGGTGACTCAGCAAGA |
| | ACACCATCACCACCATCACTAG |

| | | |
|---|---|---|
| ORF Start: at 1 | | ORF Stop: TAG at 1360 |
| SEQ ID NO: 198 | 453 aa | MW at 50860.0kD |

| | |
|---|---|
| NOV23r, | TMVSAIVLYVLLAAAAHSAFAAVIDWPTEEGKEVWDYVTVRKDAYMFWWLYYATNSCKNFSELPLVM |
| CG55078-07 | |
| Protein | WLQGGPGGSSTGFGNFEEIGPLDSDLKPRKTTWLQAASLLFVDNPVGTGFSYVNGSGAYAKDLAMVA |
| Sequence | |
| | SDMMVLLKTFFSCHKEFQTVPFYIFSESYGGKMAAGIGLELYKAIQRGTIKCNFAGVALGDSWISPV |
| | DSVLSWGPYLYSMSLLEDKGLAEVSKVAEQVLNAVNKGLYREATELWGKAEMIIEQNTDGVNFYNIL |
| | TKSTPTSTMESSLEFTQSHLVCLCQRHVRHLQRDALSQLMNGPIRKKLKIIPEDQSWGGQATNVFVN |
| | MEEDFMKPVISIVDELLEAGINVTVYNGQLDLIVDTMGQEAWVRKLKWPELPKFSQLKWKALYSDPK |
| | SLETSAFVKSYKNLAFYWILKAGHMVPSDQGDMALKMMRLVTQQEHHHHHH |

Sequence comparison of the above protein sequences yields the following sequence relationships shown in Table 23B.

TABLE 23B
Comparison of NOV23a against NOV23b through NOV23r.

| | NOV23a Residues/ | Identities/Similarities |
|---|---|---|
| Protein Sequence | Match Residues | for the Matched Region |
| NOV23b | 2 . . . 453 | 452/452 (100%) |
| | 1 . . . 452 | 452/452 (100%) |
| NOV23c | 2 . . . 453 | 452/452 (100%) |
| | 1 . . . 452 | 452/452 (100%) |
| NOV23d | 1 . . . 453 | 453/453 (100%) |
| | 1 . . . 453 | 453/453 (100%) |
| NOV23e | 1 . . . 453 | 413/453 (91%) |
| | 1 . . . 415 | 413/453 (91%) |
| NOV23f | 1 . . . 453 | 453/453 (100%) |
| | 9 . . . 461 | 453/453 (100%) |
| NOV23g | 1 . . . 453 | 453/453 (100%) |
| | 1 . . . 453 | 453/453 (100%) |
| NOV23h | 1 . . . 453 | 453/453 (100%) |
| | 1 . . . 453 | 453/453 (100%) |
| NOV23i | 1 . . . 453 | 453/453 (100%) |
| | 1 . . . 453 | 453/453 (100%) |
| NOV23j | 1 . . . 453 | 453/453 (100%) |
| | 1 . . . 453 | 453/453 (100%) |
| NOV23k | 3 . . . 453 | 451/451 (100%) |
| | 10 . . . 460 | 451/451 (100%) |
| NOV231 | 3 . . . 453 | 451/451 (100%) |
| | 10 . . . 460 | 451/451 (100%) |
| NOV23m | 28 . . . 453 | 426/426 (100%) |
| | 22 . . . 447 | 426/426 (100%) |
| NOV23n | 28 . . . 453 | 426/426 (100%) |
| | 22 . . . 447 | 426/426 (100%) |
| NOV23o | 28 . . . 453 | 426/426 (100%) |
| | 1 . . . 426 | 426/426 (100%) |
| NOV23p | 2 . . . 453 | 452/452 (100%) |
| | 1 . . . 452 | 452/452 (100%) |
| NOV23q | 28 . . . 453 | 426/426 (100%) |
| | 22 . . . 447 | 426/426 (100%) |
| NOV23r | 28 . . . 453 | 426/426 (100%) |
| | 22 . . . 447 | 426/426 (100%) |

Further analysis of the NOV23a protein yielded the following properties shown in Table 23C.

TABLE 23C
Protein Sequence Properties NOV23a

| SignalP analysis: | Cleavage site between residues 28 and 29 | | | |
|---|---|---|---|---|
| PSORT II analysis: | PSG: a new signal peptide prediction method | | | |
| | N-region: length 8; pos.chg 2; neg.chg 1 | | | |
| | H-region: length 4; peak value −1.05 | | | |
| | PSG score: −5.45 | | | |
| | GvH: von Heijne's method for signal seq. | | | |
| | recognition | | | |
| | GvH score (threshold: -2.1): 4.41 | | | |
| | possible cleavage site: between 26 and 27 | | | |
| | >>>Seems to have no N-terminal signal peptide | | | |
| | ALOM: Klein et al's method for TM region | | | |
| | allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL Likelihood = −4.78 | | | |
| | Transmembrane 14 — 30 | | | |
| | PERIPHERAL Likelihood = 1.01 (at 136) | | | |
| | ALOM score: −4.78 (number of TMSs: 1) | | | |
| | MTOP: Prediction of membrane topology | | | |
| | (Hartmamn et al.) | | | |
| | Center position for calculation: 21 | | | |
| | Charge difference: −7.0 C(−4.0) - N( 3.0) | | | |
| | N >=C: N-terminal side will be inside | | | |
| | >>>membrane topology: type 2 (cytoplasmic tail | | | |
| | 1 to 14) | | | |
| | MITDISC: discrimination of mitochondrial | | | |
| | targeting seq | | | |
| | R content: | 3 | Hyd Momeat(75): | 15.42 |
| | Hyd Moment(95): | 13.28 | G content: | 2 |
| | D/E content: | 2 | S/T content: | 2 |
| | Score: | −2.53 | | |

Gavel: prediction of cleavage sites for
mitochondrial preseg
R-2 motif at 23 PRW|LL
NUCDISC: discrimination of nuclear
localization signals
pat4: KPRK (4) at 100
pat7: PIRKKLK (5) at 317
bipartite: none
content of basic residues: 9.5%
NLS Score: 0.21
KDEL: ER retention motif in the
C-terminus: none
ER Membrane Retention Signals: none
SKL: peroxisomal targeting signal in the
C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern : none
Prenylation motif: none
memYQRL: transport motif from cell surface
to Golgi: none
Tyrosines in the tail: none
Dileucine motif in the tail: none
checking 63 PROSITE DNA binding motifs: none
checking 71 PROSITE ribosomal protein
motifs: none
checking 33 PROSITE prokaryotic DNA
binding motifs: none
NNCN: Reinhardt's method for Cytoplasmic/
Nuclear discrimination
Prediction: cytoplasmic
Reliability: 94.1
COIL: Lupas's algorithm to detect
coiled-coil regions
total: 0 residues
Final Results (k = 9/23)
33.3%: Colgi
22.2%: cytoplasmic
22.2%: mitochondrial
11.1%: extracellular, including cell wall
11.1%: endoplasmic reticulum
>>prediction for CG55078-04 is gol (k = 9)

A search of the NOV23a protein against the Geneseq database, a proprietary database that contains sequences published in patents and patent publication, yielded several homologous proteins shown in Table 23D.

TABLE 23D
Geneseq Results for NOV23a

| | | NOV23a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABP65102 | Hypoxia-induced protein #28 - | 2 . . . 453 | 452/452 (100%) | 0.0 |
| | Homo sapiens, 452 aa. | 1 . . . 452 | 452/452 (100%) | |
| | [WO200246465-A2, | | | |
| | 13-JUN-2002] | | | |
| ABB84842 | Human PRO302 protein | 2 . . . 453 | 452/452 (100%) | 0.0 |
| | sequence SEQ ID NO:52 - | 1 . . . 452 | 452/452 (100%) | |
| | Homo sapiens, 452 aa. | | | |
| | [WO200200690-A2, | | | |
| | 03-JAN-2002] | | | |
| ABB95448 | Human angiogenesis related | 2 . . . 453 | 452/452 (100%) | 0.0 |
| | protein PRO302 SEQ ID | 1 . . . 452 | 452/452 (100%) | |
| | NO: 52 - Homo sapiens, 452 | | | |
| | aa. [WO200208284-A2, | | | |
| | 31-JAN-2002] | | | |
| AAB80255 | Human PRO302 protein - | 2 . . . 453 | 452/452 (100%) | 0.0 |
| | Homo sapiens, 452 aa. | 1 . . . 452 | 452/452 (100%) | |
| | [WO200104311-A1, | | | |
| | 18-JAN-2001] | | | |
| AAB20341 | Human PRO302 - Homo | 2 . . . 453 | 452/452 (100%) | 0.0 |
| | sapiens, 452 aa. | 1 . . . 452 | 452/452 (100%) | |
| | [WO200119987-A1, | | | |
| | 22-MAR-2001] | | | |

In a BLAST search of public sequence datbases, the NOV23a protein was found to have homology to the proteins shown in the BLASTP data in Table 23E.

TABLE 23E
Public BLASTP Results for NOV23a

| | | NOV23a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9HB40 | Serine carboxypeptidase 1 | 2 . . . 453 | 452/452 (100%) | 0.0 |
| | precursor protein | 1 . . . 452 | 452/452 (100%) | |
| | (Hypothetical protein | | | |
| | FLJ14467) - Homo sapiens | | | |
| | (Human), 452 aa | | | |
| Q9H3F0 | MSTP034 - Homo sapiens | 52 . . . 453 | 402/402 (100%) | 0.0 |
| | (Human), 402 aa. | 1 . . . 402 | 402/402 (100%) | |
| Q920A6 | Retinoid-inducible serine | 2 . . . 453 | 374/452 (82%) | 0.0 |
| | carboxypeptidase precursor - | 1 . . . 452 | 415/452 (91%) | |
| | Rattus norvegicus (Rat), 452 | | | |
| | aa. | | | |
| Q9D625 | 4833411K15Rik protein - | 2 . . . 453 | 372/452 (82%) | 0.0 |
| | Mus musculus (Mouse), 452 | 1 . . . 452 | 411/452 (90%) | |
| | aa. | | | |
| Q99J29 | RIKEN cDNA 4833411K15 | 2 . . . 453 | 371/452 (82%) | 0.0 |
| | gene (Retinoid-inducible | 1 . . . 452 | 410/452 (90%) | |
| | serine caroboxypetidase) - | | | |
| | Mus musculus (Mouse), 452 | | | |
| | aa. | | | |

PFam analysis predicts that the NOV23a protein contains the domains shown in the Table 23F.

TABLE 23F
Domain Analysis of NOV23a

| | | Identities/ | |
|---|---|---|---|
| | | Similarities | |
| | NOV23a | for the Matched | |
| Pfam Domain | Match Region | Region | Expect Value |
| serine_carbpept | 43 . . . 237 | 57/212 (27%) | 1e−22 |
| | | 138/212 (65%) | |
| serine_carbpept | 338 . . . 452 | 40/119 (34%) | 9.7e−20 |
| | | 82/119 (69%) | |

### Example 24

The NOV24 clone was analyzed, and the nucleotide and encoded polypeptide sequences are shown in Table 24A.

TABLE 24A
NOV24 Sequence Analysis

| | |
|---|---|
| SEQ ID NO: 199 | 3482 bp |

| | |
|---|---|
| NOV24a, | TTATTTGACTATTTTATGGTAGGGGAGAAGGTTGAGTGTTGTTGTGAAAGCCCTGATATCAGTAATG |
| CG56149-07 | |
| DNA Sequence | GGGATGATACAATCTGCCAGCAGAGGAGAGGTTGGCAGGTAGGTCAGCTGCATCACTTCACAAGAAG |
| | AATTTGAATCCTCACTAGAAGGGGTACCATCCTCTTCCAGTTCATACTTCCCATATCCAACAACATG |
| | AACGCTGAGCATTTTACTTCCTGGCCCTCTATGGGCCTTGAACCAGTTGACCAGGTCTGATTTTGAG |
| | AATGACTTCAGTGCTTCAATCTCGTGGGCAAGGCGGTCAAAGAGGTACTGCTGTGTAACCACTTCAT |
| | TCCAGTTCCTATCCACCTCCTCCCCAAGGTGGGTATCCTCACACTCCTTCAGCTTGATGAGAGCTGT |
| | GACCTGGGTGTTGAATGCCTCTTCAGTGAGGTTCTCAATCTTCTCCTCAAAGCTAGAAAGAAACTCT |
| | TCTATCTTCTTATCAACAACTTCAGAATTGTATTTGGTTGCCTGAGTCCCCACAGTGACAGAAAATC |
| | CTAGAATCCCGGATGTGTTCCTACAGGTAGGGTAGACATGGTACCCAAGGGTCTGCTTGGTTCGAAG |
| | GAAGTCAAAACAAGGTTCTTCCATGTGCATCACAAGCAGCTCCATAAGCGTATATTCTCTTAGACTC |
| | CTGGTACCTGACTGGTAGTACACAGTGACTTCAGAGTTGGCATCACCCTTGTTCAGAGCTTTCACTT |
| | TGCATAGATGGTGGCCACTGGGCAGCTCTACCACCTGGAACTGCACAGGCATCTCCTGCTCCAGAGG |
| | CTTGAAGTTTAGTTTGTCAACAACATATTTCAGGAAATCCATAGATTCTGTGCTTGTGACATTCCCT |
| | TGTACCAGGCCCTCCACAAAGAGCTGGGATTTGAATTCTTTGACGAAGCTCAGCAGAGACTCAAGGG |
| | AAAGGCCGTCCATCAAAGCCTGGTACTTGTCAATCATAGACCAACGGGCATATTCCAGGATTAAAAG |
| | CCGTACATCTTTGGCCAAAGTCTCGGGCTTGATGAGGATGTTAAAGTAGGTCTTCTTCAACTGCTCA |
| | GTTATCATTGTAAAGACAGCTGGTGTGGAATTGAACTCAGCTAAGTAGTCAATAATGAGCTGAAACA |
| | GTAGAGGTAGTTTGTGGTTAAATCCTTTCACTCGAATAATTAAACCATGTTCTCCAGCTACCAGTTT |
| | ATACTCCAGCTGTGCCACATCTGCTTCATAAGCTGGTTCCGCAAGGTTATGCGTAAGGATATTGACA |
| | AAGATATCAAAGAGGACCACATTTGCTGCAGATTTCTGTATCAACGGTGAAATTAGATGGAAACGTA |
| | TATATGCTTTGGGGATTTTGAATTTGTTGTCTTTCTTATACCACAGGCAACCTTGTGGAGTATTCAC |
| | AATTTTAACTGGGTATTCTGTTTCCGGGCAATCGAAAGCCTTCAACGTAAAGTCCGTGGCTATGTAC |
| | TTGTTTTCAGCTGGAAGATGAAGATCTGGATTTAATTCGAAATTACTATTCCACAGTTCAGCCCAAG |
| | AGTTTTCAATATCTTCTATACTATATTGAGTTCCAAACCATTTCTCCTTGAGGTCACATTTTCCCTC |
| | ATTAGCACCAGACAGTAAAACAAGATTTGCTTTTTGAGGAACTAGCTGATTCAAGGCTTCACCAATG |
| | ACTTCTGGCTTGTATTCAAAAAGAAGCTGATCTCCAGTGAGAATGTCCTGCAATGGGTACAGCTGCA |
| | TGTTCTCACACATGTTTTCCACATACTCAACTGGATCTGTCTGTTCTTGGTAATGAAATTCATTATC |
| | CTCAATTTTCCGAATCTCTTCAAAAATTCTTTTTTTTGGGCCTAGCTTCTGCAGCATTTTTAAATAC |
| | TGAAAGACAGTGTAAGCAACCTCATAAAAATGTTCATAACCCTCATCAGTCAATGTAATAGAAATGC |
| | TGAACACTGAATAAGTAGAATTTTGCTCAAATCCTGTCTCACCATTTCCACCAAACAGTGCAAGAGC |
| | CCAGCATTTTTTCCTAAGGAAAGAAAGAATGCTGCCTTTGCCTTCATGTCCAACCAGCCAGGATATA |
| | TAATGAAGTGGCTTCACCCTGTAATGTTGCTGTTGAGGAGGAAGTGCCCATGTGATGGTCAGAGCAT |
| | GAATTTTTCTGATTGGAACAACTCTATAAAGTTTGTTAAATGCTGGTGTGTCAAATGGATCCGTTAA |
| | ATGGCCAAAGTTTGGTCTGGGTAACCCATTGTTTGGTATCTGAGAGAAGATTTCAGTCACCCACTTT |
| | TCCAAAGTATCCAGTGTTTCTTTGGATTGAACCACTAAAGTCATGTAATGAGAAGAGTAGTAACGCA |
| | TCCAGAATTCTCTCAATCTAGCATGTGTATCAATATTATTCTTTCTTGGCTCATGCTTGAGCGTCTC |
| | AGCATTTCCCCAAAAAAATTTTCCCATAGGATGTCCAGGTCTAGCAAGGCTTCCAAACAACATTTCC |
| | TTTCTGTTTGCATCAGAAGGCCTTGCAAGTTGATATTCACTATCAACAGCTTCAACTTCACCGTCAA |
| | TTGCATCTCTGATCATTAGTGCGTGGATCAAGAACTGCGCCCATCTATCAAGAGCTTCCTTGAAGTA |
| | CTTCCTCTGGACATCAAACTGAAAGACAGTGCGTTCACAATCAGTTGAGGCATTATCACTACCCCCA |
| | TGCTTCTTCAGGAAGGCATCAAATCCATTCTCATCTGGATATTTCAAACTACCCATGAATACCATGT |
| | GCTCCAAAAAGTGTGCCAGCCCCGGCAGGTCATCTGGATCAGCGAAACTCCCAACTCCAACACAAAG |
| | AGCCGCTGCAGACTGTTTTTCAGTAGTTTTTTTTCTAGCTTCTGCTCTCTCTTCTAATTCTTCCAAT |
| | TCATTATCCTCAGTATCAAGATCATCATCATGTTCATCATCAAACTCATCTTCATCATCAAAACCCT |
| | CTTCATCGTCATCTTCTATTTCAGCTCCAGAATCTTCATCATCATCTTCTTCTTCTTCCTCCACCTC |
| | CTCTTCTTCTTCATCATCTGTTGTATTTCCTGTTTTACCTTCCATATTACTTAGGTCTGAAATCAGA |
| | AGTGCCTGCAAGCCATTCTGTAATTTGATGTATCGGTATTGCTTGGGGTCGCTGGGAGACTTGACGA |
| | TCTCAGGGTCCCCAGCATTACTGAGAGACCCCCTCCGTCCCTCTTCCTCAGATTCATCCGCTCCTAG |
| | ACGGGCAACCCGGCTGTTCTCGCCCAGATCCTGTCCATTGGGCTGCAGGTCAGGGCAGCTGCAGGTA |
| | GACTTCGCCTTGTTCCTTCCAGGCATGGCCAGAATAGGAAAGGGTCTGGCAGCAGCAGAGTCTTCGC |
| | ACCGACCCCGCGTTTCGATTCCCCAGAGCGCCGCGAGCTCCCGCCCGGCCTCACACAACTTCCTCCG |
| | GGTGGCACAGACTGCAGCAACAGTGACTCTCCTCAGGTGATGGTGGTGATGGTGGCCCATGGTGG |

| | | |
|---|---|---|
| ORF Start: at 2636 | | ORF Stop: end of sequence |
| SEQ ID NO: 200 | 1159aa | MW at 132665.4 kD |

| | |
|---|---|
| NOV24a, | TMGHHHHHHLRRVTVAAVCATRRKLCEAGRELAALWGIETRGRCEDSAAARPFPILAMPGRNKAKST |
| CG56149-07 | |
| Protein Sequence | CSCPDLQPNGQDLGENSRVARLGADESEEEGRRGSLSNAGDPEIVKSPSDPKQYRYIKLQNGLQALL |
| | SDLSNMEGKTGNTTDDEEEEEVEEEEEDDDEDSGAEIEDDDEEGFDDEDEFDDEHDDDLDTEDNEL |
| | EELEERAEARKKTTEKQSAAALCVGVGSFADPDDLPGLAHFLEHMVFMGSLKYPDENGFDAFLKKHG |
| | GSDNASTDCERTVFQFDVQRKYFKEALDRWAQFFIHPLMIRDAIDREVEAVDSEYQLARPSDANRKE |
| | MLFGSLARPGHPMGKFFWGNAETLKHEPRKNNIDTHARLREFWMRYYSSHYMTLVVQSKETLDTLEK |
| | WVTEIFSQIPNNGLPRPNFGHLTDPFDTPAFNKLYRVVPIRKIHALTITWALPPQQQHYRVKPLHYI |
| | SWLVGHEGKGSILSFLRKKCWALALFGGNGETGFEQNSTYSVFSISITLTDEGYEHFYEVAYTVFQY |
| | LKMLQKLGPKKRIFEEIRKIEDNEFHYQEQTDPVEYVENMCENMQLYPLQDILTGDQLLFEYKPEVI |
| | GEALNQLVPQKANLVLLSGANEGKCDLKEKWFGTQYSIEDIENSWAELWNSNFELNPDLHLPAENKY |
| | IATDFTLKAFDCPETEYPVKIVNTPQGCLWYKKDNKFKIPKAYIRFHLISPLIQKSAANVVLFDIFV |
| | NILTHNLAEPAYEADVAQLEYKLVAGEHGLIIRVKGFNHKLPLLFQLIIDYLAEFNSTPAVFTMITE |
| | QLKKTYFNILIKPETLAKDVRLLILEYARWSMIDKYQALMDGLSLESLLSFVKEFKSQLFVEGLVQG |
| | NVTSTESMDFLKYVVDKLNFKPLEQEMPVQFQVVELPSGHHLCKVKALNKGDANSEVTVYYQSGTRS |
| | LREYTLMELLVMHMEEPCFDFLRTKQTLGYHVYPTCRNTSGILGFSVTVGTQATKYNSEVVDKKIEE |
| | FLSSFEEKIENLTEEAFNTQVTALIKLKECEDTHLGEEVDRNWNEVVTQQYLFDRLAHEIEALKSFS |
| | KSDLVNWFKAHRGPGSKMLSVHVVGYGKYELEEDGTPSSEDSNSSCEVMQLTYLPTSPLVADCIIPI |
| | TDIRAFTTTLNLLPYHKIVK |

| | |
|---|---|
| SEQ ID NO: 201 | 3647 bp |

| | |
|---|---|
| NOV24b, | AGACTCGGCTGGGGGAGGGGTTCAGGCCTGTTCCCCGCGGCTGCGGCAGCACCAGGCCCGGCCGCCA |
| CG56149-03 | |
| DNA Sequence | CCGCCTCTAGAACGCGGAGGAGGTGGGTCCTGGGAAGCGGGATGTCCATCGCTCCAGCTTGGTGGTG |
| | AATGCTGAGGAGAGTCACTGTTGCTGCAGTCTGTGCCACCCGGAGGAAGTTGTGTGAGGCCGGGCGG |
| | GACGTCGCGGCGCTCTGGGGAATCGAAACGCGGGGTCGGTGCGAAGACTCTGCTGCTGCCAGACCCT |
| | TTCCTATTCTGGCCATGCCTGGAAGGAACAAGGCGAAGTCTACCTGCAGCTGCCCTGACCTGCAGCC |
| | CAATGGACAGGATCTGGGCGAGAACAGCCGGGTTGCCCGTCTAGGAGCGGATGAATCTGAGGAAGAG |
| | GGACGGAGGGGGTCTCTCAGTAATGCTGGGGACCCTGAGATCGTCAAGTCTCCCAGCGACCCCAAGC |
| | AATACCGATACATCAAATTACAGAATGGCCTACAGGCACTTCTGATTTCAGACCTAAGTAATATGGA |
| | AGGTAAAACAGGAAATACAACAGATGATGAAGAAGAAGAGGAGGTGGAGGAAGAAGAAGAAGATGAT |
| | GATGAAGATTCTGGAGCTGAAATAGAAGATGACGATGAAGAGGGTTTTGATGATGAAGATGAGTTTG |
| | ATGATGAACATGATGATGATCTTGATACTGAGGATAATGAATTGGAAGAATTAGAAGAGAGAGCAGA |
| | AGCTAGAAAAAAAACTACTGAAAAACAGTCTGCAGCGGCTCTTTGTGTTGGAGTTGGGAGTTTCGCT |
| | GATCCAGATGACCTGCCGGGGCTGGCACACTTTTTGGAGCACATGGTATTCATGGGTAGTTTGAAAT |
| | ATCCAGATGAGAATGGATTTGATGCCTTCCTGAAGAAGCATGGGGGTAGTGATAATGCCTCAACTGA |
| | TTGTGAACGCACTGTCTTTCAGTTTGATGTCCAGAGGAAGTACTTCAAGGAAGCTCTTGATAGATGG |
| | GCGCAGTTCTTCATCCACCCACTAATGATCAGAGATGCAATTGACCGTGAAGTTGAAGCTGTTGATA |
| | GTGAATATCAACTTGCAAGGCCTTCTGATGCAAACAGAAAGGAAATGTTGTTTGGAAGCCTTGCTAG |
| | ACCTGGCCATCCTATGGGAAAATTTTTTTGGGGAAATGCTGAGACGCTCAAGCATGAGCCAAGAAAG |
| | AATAATATTGATACACATGCTAGATTGAGAGAATTCTGGATGCGTTACTACTCTTCTCATTACATGA |
| | CTTTAGTGGTTCAATCCAAAGAAACACTGGATACTTTGGAAAAGTGGGTGACTGAAATCTTCTCTCA |
| | GATACCAAACAATGGGTTACCCAGACCAAACTTTGGCCATTTAACGGATCCATTTGACACACCAGCA |
| | TTTAACAAACTTTATAGAGTTGTTCCAATCAGAAAAATTCATGCTCTGACCATCACATGGGCACTTC |
| | CTCCTCAACAGCAACATTACAGGGTGAAGCCACTTCATTATATATCCTGGCTGGTTGGACATGAAGG |
| | CAAAGGCAGCATTCTTTCTTTCCTTAGGAAAAAATGCTGGGCTCTTGCACTGTTTGGTGGAAATGGT |
| | GAGACAGGATTTGAGCAAAATTCTACTTATTCAGTGTTCAGCATTTCTATTACATTGACTCATGAGG |
| | GTTATGAACATTTTTATGAGGTTGCTTACACTGTCTTTCTGTATTTAAAAATGCTGCAGAAGCTAGG |
| | CCCAGAAAAAAGAATTTTTGAAGAGATTCGGAAAATTGAGGATAATGAATTTCATTACCAAGAACAG |
| | ACAGATCCAGTTGAGTATGTGGAAAACATGTGTGAGAACATGCAGCTGTACCCATTGCAGGACATTC |
| | TCACTGGAGATCAGCTTCTTTTTGAATACAAGCCAGAAGTCATTGGTGAAGCCTTGAATCAGCTAGT |
| | TCCTCAAAAAGCAAATCTTGTTTTACTGTCTGGTGCTAATGAGGGAAAATGTGACCTCAAGGAGAAA |
| | TGGTTTGGAACTCAATATAGTATAGAAGATATTGAAAACTCTTGGGCTGAACTGTGGAATAGTAATT |
| | TCGAATTAAATCCAGATCTTCATCTTCCAGCTGAAAACAAGTACATAGCCACGGACTTTACGTTGAA |
| | GGCTTTCGATTGCCCGGAAACAGAATACCCAGTTAAAATTGTGAATACTCCACAAGGTTGCCTGTGG |
| | TATAAGAAAGACAACAAATTCAAAATCCCCAAAGCATATATACGTTTCCATCTAATTTCACCGTTGA |
| | TACAGAAATCTGCAGCAAATGTGGTCCTCTTTGATATCTTTGTCAATATCCTTACGCATAACCTTGC |
| | GGAACCAGCTTATGAAGCAGATGTGGCACAGCTGGAGTATAAACTGGCAGCTGGAGAACATGGTTTA |
| | ATTATTCGAGTGAAAGGATTTAACCACAAACTACCTCTACTGTTTCAGCTCATTATTGACTACTTAG |
| | CTGAGTTCAATTCCACACCAGCTGTCTTTACAATGATAACTGAGCAGTTGAAGAAGACCTACTTTAA |
| | CATCCTCATCAAGCCTGAGACTTTGGCCAAAGATGTACGGCTTTTAATCTTGGAATATGCCCGTTGG |
| | TCTATGATTGACAAGTACCAGGCTTTGATGGACGGCCTTTCCCTTGAGTCTCTGCTGAGCTTCGTCA |
| | AAGAATTCAAATCCCAGCTCTTTGTGGAGGGCCTGGTACAAGGGAATGTCACAAGCACAGAATCTAT |
| | GGATTTCCTGAAATATGTTGTTGACAAACTAAACTTCAAGCCTCTGGAGCAGGAGATGCCTGTGCAG |
| | TTCCAGGTGGTAGAGCTGCCCAGTGGCCACCATCTATGCAAAGTGAAAGCTCTGAACAAGGGTGATG |
| | CCAACTCTGAAGTCACTGTGTACTACCAGTCAGGTACCAGGAGTCTAAGAGAATATACGCTTATGGA |
| | GCTGCTTGTGATGCACATGGAAGAACCTTGTTTTGACTTCCTTCGAACCAAGCAGACCCTTGGGTAC |
| | CATGTCTACCCTACCTGTAGGAACACATCCGGGATTCTAGGATTTTCTGTCACTGTGGGGACTCAGG |
| | CAACCAAATACAATTCTGAAGTTGTTGATAAGAAGATAGAAGAGTTTCTTTCTAGCTTTGAGGAGAA |
| | GATTGAGAACCTCACTGAAGAGGCATTCAACACCCAGGTCACAGCTCTCATCAAGCTGAAGGAGTGT |
| | GAGGATACCCACCTTGGGGAGGAGGTGGATAGGAACTGGAATGAAGTGGTTACACAGCAGTACCTCT |
| | TTGACCGCCTTGCCCACGAGATTGAAGCACTGAAGTCATTCTCAAAATCAGACCTGGTCAACTGGTT |
| | CAAGGCTCATAGAGGGCCAGGAAGTAAAATGCTCAGCGTTCATGTTGTTGGGTATGGGAAGTATGAA |
| | CTGGAAGAGGATGGATCCCCTTCTAGTGAGGATTCAAATTCTTCTTGTGAAGTGATGCAGCTGACCT |
| | ACCTGCCAACCTCTCCTCTGCTGGCAGATTGTATCATCCCCATTACTGATATCAGGGCTTTCACAAC |
| | AACACTCAACCTTCTCCCCTACCATAAAATAGTCAAATAAATAAACTGCAGTCACGTTGGCCTGAAA |
| | AAAAAAAAAAAAAAAAAAAAAAAAAAAAA |

| | | |
|---|---|---|
| ORF Start: ATG at 136 | | ORF Stop: TAA at 3589 |
| SEQ ID NO:202 | 1151 aa | MW at 131614.2 kD |

| | |
|---|---|
| NOV24b, | MLRRVTVAAVCATRRKLCEAGRDVAALWGIETRGRCEDSAAARPFPILAMPGRNKAKSTCSCPDLQP |
| CG56149-03 | |
| Protein Sequence | NGQDLGENSRVARLGADESEEEGRRGSLSNAGDPEIVKSPSDPKQYRYIKLQNGLQALLISDLSNME |
| | GKTGNTTDDEEEEEVEEEEEDDDEDSGAEIEDDDEEGFDDEDEFDDEHDDDLDTEDNELEELEERAE |
| | ARKKTTEKQSAAALCVGVGSFADPDDLPGLAHFLEHMVFMGSLKYPDENGFDAFLKKHGGSDNASTD |
| | CERTVFQFDVQRKYFKEALDRWAQFFIHPLMIRDAIDREVEAVDSEYQLARPSDANRKEMLFGSLAR |
| | PGHPMGKFFWGNAETLKHEPRKNNIDTHARLREFWMRYYSSHYMTLVVQSKETLDTLEKWVTEIFSQ |
| | IPNNGLPRPNFGHLTDPFDTPAFNKLYRVVPIRKIHALTITWALPPQQQHYRVKPLHYISWLVGHEG |
| | KGSILSFLRKKCWALALFGGNGETGFEQNSTYSVFSISITLTDEGYEHFYEVAYTVFLYLKMLQKLG |
| | PEKRIFEEIRKIEDNEFHYQEQTDPVEYVENMCENMQLYPLQDILTGDQLLFEYKPEVIGEALNQLV |
| | PQKANLVLLSGANEGKCDLKEKWFGTQYSIEDIENSWAELWNSNFELNPDLHLPAENKYIATDFTLK |
| | AFDCPETEYPVKIVNTPQGCLWYKKDNKFKIPKAYIRFHLISPLIQKSAANVVLFDIFVNILTHNLA |
| | EPAYEADVAQLEYKLAAGEHGLIIRVKGFNHKLPLLFQLIIDYLAEFNSTPAVFTMITEQLKKTYFN |
| | ILIKPETLAKDVRLLILEYARWSMIDKYQALMDGLSLESLLSFVKEFKSQLFVEGLVQGNVTSTESM |
| | DFLKYVVDKLNFKPLEQEMPVQFQVVELPSGHHLCKVKALNKGDANSEVTVYYQSGTRSLREYTLME |
| | LLVMHMEEPCFDFLRTKQTLGYHVYPTCRNTSGILGFSVTVGTQATKYNSEVVDKKIEEFLSSFEEK |
| | IENLTEEAFNTQVTALIKLKECEDTHLGEEVDRNWNEVVTQQYLFDRLAHEIEALKSFSKSDLVNWF |
| | KAHRGPGSKMLSVHVVGYGKYELEEDGSPSSEDSNSSCEVMQLTYLPTSPLLADCIIPITDIRAFTT |
| | TLNLLPYHKIVK |

| | |
|---|---|
| SEQ ID NO: 203 | 3851 bp |

| | |
|---|---|
| NOV24c, | AGACTGGGGTGGGGGAGGGGTTCAGGCCTGTTCCCCGCGGCTGCGGCAGCACCAGGGCCGGCCGCCA |
| CG56149-01 | |
| DNA Sequence | CCGCCTCTAGAACGCGGAGGAGGTGGGTCCTGGGAAGCGGGATGTCCATCGCTCCAGCTTGGTGGTG |
| | AATGCTGAGGAGAGTCACTGTTGCTGCAGTCTGTGCCACCCGGAGGAAGTTGTGTGAGGCCGGGCGG |
| | GACGTCGCGGCGCTCTGGGGAATCGAAACGCGGGGTCGGTGCGAAGACTCTGCTGCTGCCAGACCCT |
| | TTCCTATTCTGGCCATGCCTGGAAGGAACAAGGCGAAGTCTACCTGCAGCTGCCCTGACCTGCAGCC |
| | CAATGGACAGGATCTGGGCGAGAACAGCCGGGTTGCCCGTCTAGGAGCGGATGAATCTGAGGAAGAG |
| | GGACGGAGGGGGTCTCTCAGTAATGCTGGGGACCCTGAGATCGTCAAGTCTCCCAGCGACCCCAAGC |
| | AATACCGATACATCAAATTACAGAATGGCCTACAGGCACTTCTGATTTCAGACCTAAGTAATATGGA |
| | AGGTAAAACAGGAAATACAACAGATGATGAAGAAGAAGAGGAGGTGGAGGAAGAAGAAGAAGATGAT |
| | GATGAAGATTCTGGAGCTGAAATAGAAGATGACGATGAAGAGGGTTTTGATGATGAAGATGAGTTTG |
| | ATGATGAACATGATGATGATCTTGATACTGAGGATAATGAATTGGAAGAATTAGAAGAGAGAGCAGA |
| | AGCTAGAAAAAAAACTACTGAAAAACAGCAATTGCAGAGCCTGTTTTTGCTGTGGTCAAAGCTGACT |
| | GATAGACTGTGGTTTAAGTCAACTTATTCAAAAATGTCTTCAACCCTGCTGGTCGAGACAAGAAATC |
| | TTTATGGGGTAGTTGGAGCTGAAAGCAGGTCTGCACCTGTTCAGCATTTGGCAGGATGGCAAGCGGA |
| | GGAGCAGCAGGGTGAAACTGACACAGTTCTGTCTGCAGCGGCTCTTTGTGTTGGAGTTGGGAGTTTC |
| | GCTGATCCAGATGACCTGCCGGGGCTGGCACACTTTTTGGAGCACATGGTATTCATGGGTAGTTTGA |
| | AATATCCAGATGAGAATGGATTTGATGCCTTCCTGAAGAAGCATGGGGGTAGTGATAATGCCTCAAC |
| | TGATTGTGAACGCACTGTCTTTCAGTTTGATGTCCAGAGGAAGTACTTCAAGGAAGCTCTTGATAGA |
| | TGGGCGCAGTTCTTCATCCACCCACTAATGATCAGAGATGCAATTGACCGTGAAGTTGAAGCTGTTG |
| | ATAGTGAATATCAACTTGCAAGGCCTTCTGATGCAAACAGAAAGGAAATGTTGTTTGGAAGCCTTGC |
| | TAGACCTGGCCATCCTATGGGAAAATTTTTTTGGGGAAATGCTGAGACGCTCAAGCATGAGCCAAGA |
| | AAGAATAATATTGATACACATGCTAGATTGAGAGAATTCTGGATGCGTTACTACTCTTCTCATTACA |
| | TGACTTTAGTGGTTCAATCCAAAGAAACACTGGATACTTTGGAAAAGTGGGTGACTGAAATCTTCTC |
| | TCAGATACCAAACAATGGGTTACCCAGACCAAACTTTGGCCATTTAACGGATCCATTTGACACACCA |
| | GCATTTAACAAACTTTATAGAGTTGTTCCAATCAGAAAAATTCATGCTCTGACCATCACATGGGCAC |
| | TTCCTCCTCAACAGCAACATTACAGGGTGAAGCCACTTCATTATATATCCTGGCTGGTTGGACATGA |
| | AGGCAAAGGCAGCATTCTTTCTTTCCTTAGGAAAAAATGCTGGGCTCTTGCACTGTTTGGTGGAAAT |
| | GGTGAGACAGGATTTGAGCAAAATTCTACTTATTCAGTGTTCAGCATTTCTATTACATTGACTGATG |
| | AGGGTTATGAACATTTTTATGAGGTTGCTTACACTGTCTTTCTGTATTTAAAAATGCTGCAGAAGCT |
| | AGGCCCAGAAAAAAGAATTTTTGAAGAGATTCGGAAAATTGAGGATAATGAATTTCATTACCAAGAA |
| | CAGACAGATCCAGTTGAGTATGTGGAAAACATGTGTGAGAACATGCAGCTGTACCCATTGCAGGACA |
| | TTCTCACTGGAGATCAGCTTCTTTTTGAATACAAGCCAGAAGTCATTGGTGAAGCCTTGAATCAGCT |
| | AGTTCCTCAAAAAGCAAATCTTGTTTTACTGTCTGGTGCTAATGAGGGAAAATGTGACCTCAAGGAG |
| | AAATGGTTTGGAACTCAATATAGTATAGAAGATATTGAAAACTCTTGGGCTGAACTGTGGAATAGTA |
| | ATTTCGAATTAAATCCAGATCTTCATCTTCCAGCTGAAAACAAGTACATAGCCACGGACTTTACGTT |
| | GAAGGCTTTCGATTGCCCGGAAACAGAATACCCAGTTAAAATTGTGAATACTCCACAAGGTTGCCTG |
| | TGGTATAAGAAAGACAACAAATTCAAAATCCCCAAAGCATATATACGTTTCCATCTAATTTCACCGT |
| | TGATACAGAAATCTGCAGCAAATGTGGTCCTCTTTGATATCTTTGTCAATATCCTTACGCATAACCT |
| | TGCGGAACCAGCTTATGAAGCAGATGTGGCACAGCTGGAGTATAAACTGGCAGCTGGAGAACATGGT |
| | TTAATTATTCGAGTGAAAGGATTTAACCACAAACTACCTCTACTGTTTCAGCTCATTATTGACTACT |
| | TAGCTGAGTTCAATTCCACACCAGCTGTCTTTACAATGATAACTGAGCAGTTGAAGAAGACCTACTT |
| | TAACATCCTCATCAAGCCTGAGACTTTGGCCAAAGATGTACGGCTTTTAATCTTGGAATATGCCCGT |
| | TGGTCTATGATTGACAAGTACCAGGCTTTGATGGACGGCCTTTCCCTTGAGTCTCTGCTGAGCTTCG |
| | TCAAAGAATTCAAATCCCAGCTCTTTGTGGAGGGCCTGGTACAAGGGAATGTCACAAGCACAGAATC |
| | TATGGATTTCCTGAAATATGTTGTTGACAAACTAAACTTCAAGCCTCTGGAGCAGGAGATGCCTGTG |
| | CAGTTCCAGGTGGTAGAGCTGCCCAGTGGCCACCATCTATGCAAAGTGAAAGCTCTGAACAAGGGTG |
| | ATGCCAACTCTGAAGTCACTGTGTACTACCAGTCAGGTACCAGGAGTCTAAGAGAATATACGCTTAT |
| | GGAGCTGCTTGTGATGCACATGGAAGAACCTTGTTTTGACTTCCTTCGAACCAAGCAGACCCTTGGG |
| | TACCATGTCTACCCTACCTGTAGGAACACATCCGGGATTCTAGGATTTTCTGTCACTGTGGGGACTC |
| | AGGCAACCAAATACAATTCTGAAGTTGTTGATAAGAAGATAGAAGAGTTTCTTTCTAGCTTTGAGGA |
| | GAAGATTGAGAACCTCACTGAAGAGGCATTCAACACCCAGGTCACAGCTCTCATCAAGCTGAAGGAG |
| | TGTGAGGATACCCACCTTGGGGAGGAGGTGGATAGGAACTGGAATGAAGTGGTTACACAGCAGTACC |
| | TCTTTGACCGCCTTGCCCACGAGATTGAAGCACTGAAGTCATTCTCAAAATCAGACCTGGTCAACTG |
| | GTTCAAGGCTCATAGAGGGCCAGGAAGTAAAATGCTCAGCGTTCATGTTGTTGGGTATGGGAAGTAT |
| | GAACTGGAAGAGGATGGATCCCCTTCTAGTGAGGATTCAAATTCTTCTTGTGAAGTGATGCAGCTGA |
| | CCTACCTGCCAACCTCTCCTCTGCTGGCAGATTGTATCATCCCCATTACTGATATCAGGGCTTTCAC |
| | AACAACACTCAACCTTCTCCCCTACCATAAAATAGTCAAATAAATAAACTGCAGTCACGTTGGCCTG |
| | AAAAAAAAAAAAAAAAAAAAAAAAAAAAAAAA |

| | | |
|---|---|---|
| ORF Start: ATG at 136 | | ORF Stop: TAA at 3793 |
| SEQ ID NO: 204 | 1219aa | MW at 139326.8 kD |

| | |
|---|---|
| NOV24c, | MLRRVTVAAVCATRRKLCEAGRDVAALWGIETRGRCEDSAAARPFPILAMPGRNKAKSTCSCPDLQP |
| CG56149-01 | |
| Protein Sequence | NGQDLGENSRVARLGADESEEEGRRGSLSNAGDPEIVKSPSDPKQYRYIKLQNGLQALLISDLSNME |
| | GKTGNTTDDEEEEEVEEEEEDDDEDSGAEIEDDDEEGFDDEDEFDDEHDDDLDTEDNELEELEERAE |
| | ARKKTTEKQQLQSLFLLWSKLTDRLWFKSTYSKMSSTLLVETRNLYGVVGAESRSAPVQHLAGWQAE |
| | EQQGETDTVLSAAALCVGVGSFADPDDLPGLAHFLEHMVFMGSLKYPDENGFDAFLKKHGGSDNAST |
| | DCERTVFQFDVQRKYFKEALDRWAQFFIHPLMIRDAIDREVEAVDSEYQLARPSDANRKEMLFGSLA |
| | RPGHPMGKFFWGNAETLKHEPRKNNIDTHARLREFWMRYYSSHYMTLVVQSKETLDTLEKWVTEIFS |
| | QIPNNGLPRPNFGHLTDPFDTPAFNKLYRVVPIRKIHALTITWALPPQQQHYRVKPLHYISWLVGHE |
| | GKGSILSFLRKKCWALALFGGNGETGFEQNSTYSVFSISITLTDEGYEHFYEVAYTVPLYLKMLQKL |
| | GPEKRIFEEIREIEDNEFHYQEQTDPVEYVENNCENMQLYPLQDILTGDQLLFEYKPEVIGEALNQL |
| | VPQKANLVLLSGANEGKCDLKEKWFGTQYSIEDIENSWAELWNSNFELNPDLHLPAENKYIATDFTL |
| | KAFDCPETEYPVKIVNTPQGCLWYKKDNKPKIPKAYIRFHLISPLIQKSAANVVLFDIFVNILTHNL |
| | AEPAYEADVAQLEYKLAAGEHGLIIRVKGFNHKLPLLFQLIIDYLAEFNSTPAVFTMITEQLKKTYP |
| | NILIKPETLAKDVRLLILEYARWSMIDKYQALMDGLSLESLLSFVKEFKSQLFVEGLVQGNVTSTES |
| | MDFLKYVVDKLNPKPLEQEMPVQFQVVELPSGHHLCKVKALNKGDANSEVTVYYQSGTRSLREYTLM |
| | ELLVMHMEEPCFDFLRTKQTLGYHVYPTCRNTSGILGFSVTVGTQATKYNSEVVDKKIEEFLSSFEE |
| | KIENLTEEAFNTQVTALIKLKECEDTHLGEEVDRNWNEVVTQQYLPDPIAHEIEALKSFSKSDLVNW |
| | FKAHRGPGSKMLSVHVVGYGKYELEEDGSPSSEDSNSSCEVMQLTYLPTSPLLADCIIPITDIRAFT |
| | TTLNLLPYHKIVK |

| | |
|---|---|
| SEQ ID NO: 205 | 4315 bp |

| | |
|---|---|
| NOV24d, | ACGCGTGAGGGAGACTGGGGTGGGGGAGGGGTTCAGGCCTGTTCCCCGCGGCTGCGGCAGCACCAGG |
| CG56149-02 | |
| DNA Sequence | GCCGGCCGCCACCGCCTCTAGAACGCGGAGGAGGTGGGTCCTGGGAAGCGGGATGTCCATCGCTCCA |
| | GCTTGGTGGTGAATGCTGAGGAGAGTCACTGTTGCTGCAGTCTGTGCCACCCGGAGGAAGTTGTGTG |
| | AGGCCGGGCGGGAGCTCGCGGCGCTCTGGGGAATCGAAACGCGGGGTCGGTGCGAAGACTCTGCTGC |
| | TGCCAGACCCTTTCCTATTCTGGCCATGCCTGGAAGGAACAAGGCGAAGTCTACCTGCAGCTGCCCT |
| | GACCTGCAGCCCAATGGACAGGATCTGGGCGAGAACAGCCGGGTTGCCCGTCTAGGAGCGGATGAAT |
| | CTGAGGAAGAGGGACGGAGGGGGTCTCTCAGTAATGCTGGGGACCCTGAGATCGTCAAGTCTCCCAG |
| | CGACCCCAAGCAATACCGATACATCAAATTACAGAATGGCTTGCAGGCACTTCTGATTTCAGACCTA |
| | AGTAATATGGAAGGTAAAACAGGAAATACAACAGATGATGAAGAAGAAGAGGAGGTGGAGGAAGAAG |
| | AAGAAGATGATGATGAAGATTCTGGAGCTGAAATAGAAGATGCTGAAATAGAAGATGACGATGAAGA |
| | GGGTTTTGATGATGAAGATGAGTTTGATGATGAACATGATGATGATCTTGATACTGAGGATAATGAA |
| | TTGGAAGAATTAGAAGAGAGAGCAGAAGCTAGAAAAAAAACTACTGAAAAACAGTCTGCAGCGGCTC |
| | TTTGTGTTGGAGTTGGGAGTTTCGCTGATCCAGATGACCTGCCGGGGCTGGCACACTTTTTGGAGCA |
| | CATGGTATTCATGGGTAGTTTGAAATATCCAGATGAGAATGGATTTGATGCCTTCCTGAAGAAGCAT |
| | GGGGGTAGTGATAATGCCTCAACTGATTGTGAACGCACTGTCTTTCAGTTTGATGTCCAGAGGAAGT |
| | ACTTCAAGGAAGCTCTTGATAGATGGGCGCAGTTCTTCATCCACCCACTAATGATCAGAGATGCAAT |
| | TGACCGTGAAGTTGAAGCTGTTGATAGTGAATATCAACTTGCAAGGCCTTCTGATGCAAACAGAAAG |
| | GAAATGTTGTTTGGAAGCCTTGCTAGACCTGGCCATCCTATGGGAAAATTTTTTTGGGGAAATGCTG |
| | AGACGCTCAAGCATGAGCCAAGAAAGAATAATATTGATACACATGCTAGATTGAGAGAATTCTGGAT |
| | GCGTTACTACTCTTCTCATTACATGACTTTAGTGGTTCAATCCAAAGAAACACTGGATACTTTGGAA |
| | AAGTGGGTGACTGAAATCTTCTCTCAGATACCAAACAATGGGTTACCCAGACCAAACTTTGGCCATT |
| | TAACGGATCCATTTGACACACCAGCATTTAACAAACTTTATAGAGTTGTTCCAATCAGAAAAATTCA |
| | TGCTCTGACCATCACATGGGCACTTCCTCCTCAACAGCAACATTACAGGGTGAAGCCACTTCATTAT |
| | ATATCCTGGCTGGTTGGACATGAAGGCAAAGGCAGCATTCTTTCTTTCCTTAGGAAAAAATGCTGGG |
| | CTCTTGCACTGTTTGGTGGAAATGGTGAGACAGGATTTGAGCAAAATTCTACTTATTCAGTGTTCAG |
| | CATTTCTATTACATTGACTGATGAGGGTTATGAACATTTTTATGAGGTTGCTTACACTGTCTTTCAG |
| | TATTTAAAAATGCTGCAGAAGCTAGGCCCAGAAAAAAGAATTTTTGAAGAGATTCGGAAAATTGAGG |
| | ATAATGAATTTCATTACCAAGAACAGACAGATCCAGTTGAGTATGTGGAAAACATGTGTGAGAACAT |
| | GCAGCTGTACCCATTGCAGGACATTCTCACTGGAGATCAGCTTCTTTTTGAATACAAGCCAGAAGTC |
| | ATTGGTGAAGCCTTGAATCAGCTAGTTCCTCAAAAAGCAAATCTTGTTTTACTGTCTGGTGCTAATG |
| | AGGGAAAATGTGACCTCAAGGAGAAATGGTTTGGAACTCAATATAGTATAGAAGATATTGAAAACTC |
| | TTGGGCTGAACTGTGGAATAGTAATTTCGAATTAAATCCAGATCTTCATCTTCCAGCTGAAAACAAG |
| | TACATAGCCACGGACTTTACGTTGAAGGCTTTCGATTGCCCGGAAACAGAATACCCAGTTAAAATTG |
| | TGAATACTCCACAAGGTTGCCTGTGGTATAAGAAAGACAACAAATTCAAAATCCCCAAAGCATATAT |
| | ACGTTTCCATCTAATTTCACCGTTGATACAGAAATCTGCAGCAAATGTGGTCCTCTTTGATATCTTT |
| | GTCAATATCCTTACGCATAACCTTGCGGAACCAGCTTATGAAGCAGATGTGGCACAGCTGGAGTATA |
| | AACTGGTAGCTGGAGAACATGGTTTAATTATTCGAGTGAAAGGATTTAACCACAAACTACCTCTACT |
| | GTTTCAGCTCATTATTGACTACTTAGCTGAGTTCAATTCCACACCAGCTGTCTTTACAATGATAACT |
| | GAGCAGTTGAAGAAGACCTACTTTAACATCCTCATCAAGCCTGAGACTTTGGCCAAAGATGTACGGC |
| | TTTTAATCTTGGAATATGCCCGTTGGTCTATGATTGACAAGTACCAGGCTTTGATGGACGGCCTTTC |
| | CCTTGAGTCTCTGCTGAGCTTCGTCAAAGAATTCAAATCCCAGCTCTTTGTGGAGGGCCTGGTACAA |
| | GGGAATGTCACAAGCACAGAATCTATGGATTTCCTGAAATATGTTGTTGACAAACTAAACTTCAAGC |
| | CTCTGGAGCAGGAGATGCCTGTGCAGTTCCAGGTGGTAGAGCTGCCCAGTGGCCACCATCTATGCAA |
| | AGTGAAAGCTCTGAACAAGGGTGATGCCAACTCTGAAGTCACTGTGTACTACCAGTCAGGTACCAGG |
| | AGTCTAAGAGAATATACGCTTATGGAGCTGCTTGTGATGCACATGGAAGAACCTTGTTTTGACTTCC |
| | TTCGAACCAAGCAGACCCTTGGGTACCATGTCTACCCTACCTGTAGGAACACATCCGGGATTCTAGG |
| | ATTTTCTGTCACTGTGGGGACTCAGGCAACCAAATACAATTCTGAAGTTGTTGATAAGAAGATAGAA |
| | GAGTTTCTTTCTAGCTTTGAGGAGAAGATTGAGAACCTCACTGAAGAGGCATTCAACACCCAGGTCA |
| | CAGCTCTCATCAAGCTGAAGGAGTGTGAGGATACCCACCTTGGGGAGGAGGTGGATAGGAACTGGAA |
| | TGAAGTGGTTACACAGCAGTACCTCTTTGACCGCCTTGCCCACGAGATTGAAGCACTGAAGTCAATC |
| | TCAAAATCAGACCTGGTCAACTGGTTCAAGGCCCATAGAGGGCCAGGAAGTAAAATGCTCAGCGTTC |
| | ATGTTGTTGGATATGGGAAGTATGAACTGGAAGAGGATGGTACCCCTTCTAGTGAGGATTCAAATTC |
| | TTCTTGTGAAGTGATGCAGCTGACCTACCTGCCAACCTCTCCTCTGCTGGCAGATTGTATCATCCCC |
| | ATTACTGATATCAGGGCTTTCACAACAACACTCAACCTTCTCCCCTACCATAAAATAGTCAAATAAA |
| | TAAACTGCAGTCACGTTGGCCTGAAGCAATGTGTATTTTAAAATGTGTGTGTTTGTATTTTATGGAG |
| | TTAGTTATACTACTGCCTTAGGGCTTCCATTGAAGTTTTGCACTGGCATCATAGCATTTGATTTACT |
| | TTTTATCCTTTGTTGAGACTAATAAACCCAGGGTTACTGTAGGAGCTGGCAAAGGAAAATTAGCAGA |
| | ATGGGCCAAGCGAGACCAGAAAGCCTGCAGCAGCACTTTGAGAAGCCCTGGCCTGTGTCCTCTCAGA |
| | CTGAGAATCTACTTCTTGAAAGGCCTTACGTGACCAGTATATTGAATAACTAACTAAATGCTAGGTA |
| | CTAATACCTGTTTTTTTAATGTATTTTTAAATAAAAAAGATGATAGATAGATAGATAGATATAGTTC |
| | TGTATTTCCCTTCAGAATGAGCCATCTGCTGCTGTGGCATTCATTTTATTCTATCTATCTATCTATT |
| | TTTGTTCACTGTGGGGTGGGGATCTATAAATACACACTCTTCCCAAACCCTCTAAGGCAATAAAACA |
| | TTTTTGGATAAAATGTTGGTAGGCAGCCCTACATGTGCAATATGAGTTAAGTGAAGATTCTGGGGAA |
| | TTGCCTGGCAGGGGCTAAAGACAGAACATACAATCTGACAGAGGAAAAGAATGGATCCTCCATTATT |
| | TCAAGTGTCTTTCTTTGAAAAGCTAGC |

| | | |
|---|---|---|
| ORF Start: ATG at 147 | | ORF Stop: TAA at 3615 |
| SEQ ID NO: 206 | 1156 aa | MW at 132256.8 kD |

| | |
|---|---|
| NOV24d, | MLRRVTVAAVCATRRKLCEAGRELAALWGIETRGRCEDSAAARPFPILAMPGRNKAKSTCSCPDLQP |
| CG56149-02 | |
| Protein Sequence | NGQDLGENSRVARLGADESEEEGRRGSLSNAGDPEIVKSPSDPKQYRYIKLQNGLQALLISDLSNME |
| | GKTGNTTDDEEEEEVEEEEEDDDEDSGAEIEDAEIEDDDEEGFDDEDEFDDEHDDDLDTEDNELEEL |
| | EERAEARKKTTEKQSAAALCVGVGSFADPDDLPGLAHFLEHMVFMGSLKYPDENGFDAFLKKHGGSD |
| | NASTDCERTVFQFDVQRKYFKEALDRWAQFFIHPLMIRDAIDREVEAVDSEYQLARPSDANRKEMLF |
| | GSLARPGHPMGKFFWGNAETLKHEPRKNNIDTHARLREFWMRYYSSHYMTLVVQSKETLDTLEKWVT |
| | EIFSQIPNNGLPRPNFGHLTDPFDTPAFNKLYRVVPIRKIHALTITWALPPQQQHYRVKPLHYISWL |
| | VGHEGKGSILSFLRKKCWALALFGGNGETGFEQNSTYSVFSISITLTDEGYEHFYEVAYTVFQYLKM |
| | LQKLGPEKRIFEEIRKIEDNEFHYQEQTDPVEYVENMCENMQLYPLQDILTGDQLLFEYKPEVIGEA |
| | LNQLVPQKANLVLLSGANEGKCDLKEKWFGTQYSIEDIENSWAELWNSNFELNPDLHLPAENKYIAT |
| | DFTLKAFDCPETEYPVKIVNTPQGCLWYKKDNKFKIPKAYIRFHLISPLIQKSAANVVLFDIFVNIL |
| | THNLAEPAYEADVAQLEYKLVAGEHGLIIRVKGFNHKLPLLFQLIIDYLAEFNSTPAVFTMITEQLK |
| | KTYFNILIKPETLAKDVRLLILEYARWSMIDKYQALMDGLSLESLLSFVKEFKSQLFVEGLVQGNVT |
| | STESMDFLKYVVDKLNFKPLEQEMPVQFQVVELPSGHHLCKVKALNKGDANSEVTVYYQSGTRSLRE |
| | YTLMELLVMHMEEPCFDFLRTKQTLGYHVYPTCRNTSGILGFSVTVGTQATKYNSEVVDKKIEEFLS |
| | SFEEKIENLTEEAFNTQVTALIKLKECEDTHLGEEVDRNWNEVVTQQYLFDRLAHEIEALKSESKSD |
| | LVNWFKAHRGPGSKMLSVHVVGYGKYELEEDGTPSSEDSNSSCEVMQLTYLPTSPLLADCIIPITDI |
| | RAFTTTLNLLPYHKIVK |

| | |
|---|---|
| SEQ ID NO: 207 | 3601bp |

| | |
|---|---|
| NOV24e, | GGAGGGGTTCAGGCCTGTTCCCCGCGGCTGCGGCAGCACCAGGGCCGGCCGCCACCGCCTCTAGAAC |
| CG56149-04 | |
| DNA Sequence | GCGGAGGAGGTGGGTCCTGGGAAGCGGGATGTCCATCGCTCCAGCTTGGTGGTGA+E,uns ATGCTGAGGAGA |
| | GTCACTGTTGCTCCAGTCTGTGCCACCCGGACGAAGTTGTGTGAGGCCGGGCGGGAGCTCGCGGCGC |
| | TCTGGGGAATCGAAACGCGGGGTCGGTGCGAAGACTCTGCTGCTGCCAGACCCTTTCCTATTCTGGC |
| | CATGCCTGGAAGGAACAAGGCGAAGTCTACCTGCAGCTGCCCTGACCTGCAGCCCAATGGACAGGAT |
| | CTGGGCGAGAACAGCCGGGTTGCCCGTCTAGGAGCGGATGAATCTGAGGAAGAGGGACGGAGGGGGT |
| | CTCTCAGTAATGCTGGGGACCCTGAGATCGTCAAGTCTCCCAGCGACCCCAAGCAATACCGATACAT |
| | CAAATTACAGAATGGCTTGCAGGCACTTCTGATTTCAGACCTAAGTAATATGGAAGGTAAAACAGGA |
| | AATACAACAGATGATGAAGAAGAAGAGGAGGTGGAGGAAGAAGAAGAAGATGATGATGAAGATTCTG |
| | GAGCTGAAATAGAAGATGACGATGAAGAGGGTTTTGATGATGAAGATGAGTTTGATGATGAACATGA |
| | TGATGATCTTGATACTGAGGATAATGAATTGGAAGAATTAGAAGAGAGAGCAGAAGCTAGAAAAAAA |
| | ACTACTGAAAAACAGTCTGCAGCGGCTCTTTGTGTTGGAGTTGGGAGTTTCGCTGATCCAGATGACC |
| | TGCCGGGGCTGGCACACTTTTTGGAGCACATGGTATTCATGGGTAGTTTGAAATATCCAGATGAGAA |
| | TGGATTTGATGCCTTCCTGAAGAAGCATGGGGGTAGTGATAATGCCTCAACTGATTGTGAACGCACT |
| | GTCTTTCAGTTTGATGTCCAGAGGAAGTACTTCAAGGAAGCTCTTGATAGATGGGCGCAGTTCTTCA |
| | TCCACCCACTAATGATCAGAGATGCAATTGACCGTGAAGTTGAAGCTGTTGATAGTGAATATCAACT |
| | TGCAAGGCCTTCTGATGCAAACAGAAAGGAAATGTTGTTTGGAAGCCTTGCTAGACCTGGACATCCT |
| | ATGGGAAAATTTTTTTGGGGAAATGCTGAGACGCTCAAGCATGAGCCAAGAAAGAATAATATTGATA |
| | CACATGCTAGATTGAGAGAATTCTGGATGCGTTACTACTCTTCTCATTACATGACTTTAGTGGTTCA |
| | ATCCAAAGAAACACTGGATACTTTGGAAAAGTGGGTGACTGAAATCTTCTCTCAGATACCAAACAAT |
| | GGGTTACCCAGACCAAACTTTGGCCATTTAACGGATCCATTTGACACACCAGCATTTAACAAACTTT |
| | ATAGAGTTGTTCCAATCAGAAAAATTCATGCTCTGACCATCACATGGGCACTTCCTCCTCAACAGCA |
| | ACATTACAGGGTGAAGCCACTTCATTATATATCCTGGCTGGTTGGACATGAAGGCAAAGGCAGCATT |
| | CTTTCTTTCCTTAGGAAAAAATGCTGGGCTCTTGCACTGTTTGGTGGAAATGGTGAGACAGGATTTG |
| | AGCAAAATTCTACTTATTCAGTGTTCAGCATTTCTATTACATTGACTGATGAGGGTTATGAACATTT |
| | TTATGAGGTTGCTTACACTGTCTTTCAGTATTTAAAAATGCTGCAGAAGCTAGGCCCAAAAAAAAGA |
| | ATTTTTGAAGAGATTCGGAAAATTGAGGATAATGAATTTCATTACCAAGAACAGACAGATCCAGTTG |
| | AGTATGTGGAAAACATGTGTGAGAACATGCAGCTGTACCCATTGCAGGACATTCTCACTGGAGATCA |
| | GCTTCTTTTTGAATACAAGCCAGAAGTCATTGGTGAAGCCTTGAATCAGCTAGTTCCTCAAAAAGCA |
| | AATCTTGTTTTACTGTCTGGTGCTAATGAGGGAAAATGTGACCTCAAGGAGAAATGGTTTGGAACTC |
| | AATATAGTATAGAAGATATTGAAAACTCTTGGGCTGAACTGTGGAATAGTAATTTCGAATTAAATCC |
| | AGATCTTCATCTTCCAGCTGAAAACAAGTACATAGCCACGGACTTTACGTTGAAGGCTTTCGATTGC |
| | CCGGAAACAGAATACCCAGTTAAAATTGTGAATACTCCACAAGGTTGCCTGTGGTATAAGAAAGACA |
| | ACAAATTCAAAATCCCCAAAGCATATATACGTTTCCATCTAATTTCACCGTTGATACAGAAATCTGC |
| | AGCAAATGTGGTCCTCTTTGATATCTTTGTCAATATCCTTACGCATAACCTTGCGGAACCAGCTTAT |
| | GAAGCAGATGTGGCACAGCTGGAGTATAAACTGGTAGCTGGAGAACATGGTTTAATTATTCGAGTGA |
| | AAGGATTTAACCACAAACTACCTCTACTGTTTCAGCTCATTATTGACTACTTAGCTGAGTTCAATTC |
| | CACACCAGCTGTCTTTACAATGATAACTGAGCAGTTGAAGAAGACCTACTTTAACATCCTCATCAAG |
| | CCCGAGACTTTGGCCAAAGATGTACGGCTTTTAATCCTGGAATATGCCCGTTGGTCTATGATTGACA |
| | AGTACCAGGCTTTGATGGACGGCCTTTCCCTTGAGTCTCTGCTGAGCTTCGTCAAAGAATTCAAATC |
| | CCAGCTCTTTGTGGAGGGCCTGGTACAAGGGAATGTCACAAGCACAGAATCTATGGATTTCCTGAAA |
| | TATGTTGTTGACAAACTAAACTTCAAGCCTCTGGAGCAGGAGATGCCTGTGCAGTTCCAGGTGGTAG |
| | AGCTGCCCAGTGGCCACCATCTATGCAAAGTGAAAGCTCTGAACAAGGGTGATGCCAACTCTGAAGT |
| | CACTGTGTACTACCAGTCAGGTACCAGGAGTCTAAGAGAATATACGCTTATGGAGCTGCTTGTGATG |
| | CACATGGAAGAACCTTGTTTTGACTTCCTTCGAACCAAGCAGACCCTTGGGTACCATGTCTACCCTA |
| | CCTGTAGGAACACATCCGGGATTCTAGGATTTTCTGTCACTGTGGGGACTCAGGCAACCAAATACAA |
| | TTCTGAAGTTGTTGATAAGAAGATAGAAGAGTTTCTTTCTAGCTTTGAGGAGAAGATTGAGAACCTC |
| | ACTGAAGAGGCATTCAACACCCAGGTCACAGCTCTCATCAAGCTGAAGGAGTGTGAGGATACCCACC |
| | TTGGGGAGGAGGTGGATAGGAACTGGAATGAAGTGGTTACACAGCAGTACCTCTTTGACCGCCTTGC |
| | CCACGAGATTGAAGCACTGAAGTCATTCTCAAAATCAGACCTGGTCAACTGGTTCAAGGCCCATAGA |
| | GGGCCAGGAAGTAAAATGCTCAGCGTTCATGTTGTTGGATATGGGAAGTATGAACTGGAAGAGGATG |
| | GTACCCCTTCTAGTGAGGATTCAAATTCTTCTTGTGAAGTGATGCAGCTGACCTACCTGCCAACCTC |
| | TCCTCTGCTGGCAGATTGTATCATCCCCATTACTGATATCAGGGCTTTCACAACAACACTCAACCTT |
| | CTCCCCTACCATAAAATAGTCAAATAAATAAACTGCAGTCACGTTGGCCT |

| | | |
|---|---|---|
| ORF Start: ATG at 123 | | ORF Stop: TAA at 3576 |
| SEQ ID NO: 208 | 11151 aa | MW at 131698.4 kD |

| | |
|---|---|
| NOV24e, | MLRRVTVAAVCATRRKLCEAGRELAALWGIETRGRCEDSAAARPFPILAMPGRNKAKSTCSCPDLQP |
| CG56149-04 | |
| Protein Sequence | NGQDLGENSRVARLGADESEEEGRRGSLSNAGDPEIVKSPSDPKQYRYIKLQNGLQALLISDLSNME |
| | GKTGNTTDDEEEEEVEEEEEDDDEDSGAEIEDDDEEGFDDEDEFDDEHDDDLDTEDNELEELEERAE |
| | ARKKTTEKQSAAALCVGVGSFADPDDLPGLAHFLEHMVFMGSLKYPDENGFDAFLKKHGGSDNASTD |
| | CERTVFQFDVQRKYFKEALDRWAQFFIHPLMIRDAIDREVEAVDSEYQLARPSDANRKEMLFGSLAR |
| | PGHPMGKFFWGNAETLKHEPRKNNIDTHARLREFWMRYYSSHYMTLVVQSKETLDTLEKWVTEIFSQ |
| | IPNNGLPRPNFGHLTDPFDTPAFNKLYRVVPIRKIHALTITWALPPQQQHYRVKPLHYISWLVGHEG |
| | KGSILSFLRKKCWALALFGGNGETGFEQNSTYSVFSISITLTDEGYEHFYEVAYTVFQYLKMLQKLG |
| | PKKRIFEEIRKIEDNEFHYQEQTDPVEYVENMCENMQLYPLQDILTGDQLLFEYKPEVIGEALNQLV |
| | PQKANLVLLSGANEGKCDLKEKWFGTQYSIEDIENSWAELWNSNFELNPDLHLPAENKYIATDFTLK |
| | AFDCPETEYPVKIVNTPQGCLWYKKDNKFKIPKAYIRFHLISPLIQKSAANVVLFDIFVNILTHNLA |
| | EPAYEADVAQLEYKLVAGEHGLIIRVKGFNHKLPLLFQLIIDYLAEFNSTPAVFTMITEQLKKTYFN |
| | ILIKPETLAKDVRLLILEYARWSMIDKYQALMDGLSLESLLSFVKEFKSQLFVEGLVQGNVTSTESM |
| | DFLKYVVDKLNFKPLEQEMPVQFQVVELPSGHHLCKVKALNKGDANSEVTVYYQSGTRSLREYTLME |
| | LLVMHMEEPCFDFLRTKQTLGYHVYPTCRNTSGILGFSVTVGTQATKYNSEVVDKKIEEFLSSFEEK |
| | IENLTEEAFNTQVTALIKLKECEDTHLGEEVDRNWNEVVTQQYLFDRLAHEIEALKSFSKSDLVNWF |
| | KAHRGPGSKMLSVHVVGYGKYELEEDGTPSSEDSNSSCEVMQLTYLPTSPLLADCIIPITDIRAFTT |
| | TLNLLPYHKIVK |

| | |
|---|---|
| SEQ ID NO:209 | 388 bp |

| | |
|---|---|
| NOV24f, | GGATCCTCTGCAGCGGCTCTTTGTGTTGGAGTTGGGAGTTTCGCTGATCCAGATGACCTGCCGGGGC |
| CG56149-05 | |
| DNA Sequence | TGGCACACTTTTTGGAGCACATGGTATTCATGGGTAGTTTGAAATATCCAGATGAGAATGGATTTGA |
| | TGCCTTCCTGAAGAAGCATGGGGGTAGTGATAATGCCTCAACTGATTGTGAACGCACTGTCTTTCAG |
| | TTTGATGTCCAGAGGAAGTACTTCAAGGAAGCTCTTGATAGATGGGCGCAGTTCTTCATCCACCCAC |
| | TAATGATCAGAGATGCAATTGACCGTGAAGTTGAAGCTGTTGATAGTGAATATCAACTTGCAAGGCC |
| | TTCTGATGCAAACAGAAAGGAAATGTTGTTTGGAAGCCTTGCTAGACCTGGCC |

| | | |
|---|---|---|
| ORF Start: at 7 | | ORF Stop: at 388 |
| SEQ ID NO: 210 | 127 AA | MW at 14262.8 kD |

| | |
|---|---|
| NOV24f, | SAAALCVGVGSFADPDDLPGLAHFLEHMVFMGSLKYPDENGFDAFLKKHGGSDNASTDCERTVFQFD |
| CG56149-05 | |
| Protein | VQRKYFKEALDRWAQFFIHPLMIRDAIDREVEAVDSEYQLARPSDANRKEMLFGSLARPG |
| Sequence | |

| | |
|---|---|
| SEQ ID NO: 211 | 742 bp |

| | |
|---|---|
| NOV24g, | GGCACGAGGGCGGCTGCGGCAGCACCAGGGCCGGCCGCCACCGCCTCTAGAACGCGGAGGAGGTGGG |
| CG56149-06 | |
| DNA Sequence | TCCTGGGAAGCGGGATGTCCATCGCTCCAGCTTGGTGGTGAATGCTGAGGAGAGTCACTGTTGCTGC |
| | ACGCGGGGTCGGTGCGAAGACTCTGCTGCTGCCAGACCCTTTCCTATTCTGGGCGAGAACAGCCGGG |
| | TTGCCCGTCTAGGAGCGGATGAATCTGAGGAAGAGGGACGGAGGGGGTCTCTCAGTAATGCTGGGGA |
| | CCCTGAGATCGTCAAGTCTCCCAGCGACCCCAAGCAATACCGATACATCAAATTACAGAATGGCTTG |
| | CAGGCACTTCTGATTTCAGACCTGGTCAACTGGTTCAAGGCCCATAGAGGGCCAGGAAGTGGAATGC |
| | TCAGCGTTCATGTTGTTGGATATGGGAAGTATGAACTGGAAGAGGATGGTACCCCTTCTAGTGAGGA |
| | TTCAAATTCTTCTTGTGAAGTGATGCAGCTGACCTACCTGCCAACCTCTCCTCTGCTGGCAGATTGT |
| | ATCATCCCCATTACTGATATCAGGGCTTTCACAACAACACTCAACCTTCTCCCCTACCATAAAATAG |
| | TCAAATAAATAAACTGCAGTCACGTTGGCCTGAAAAAAAAAAAAAAAAAAAAAAAAAAAAAAAAAAA |
| | AAAAA |

| | | |
|---|---|---|
| ORF Start: ATG at 109 | | ORF Stop: TAA at 676 |
| SEQ ID NO: 212 | 189 aa | MW at 20804.4 kD |

| | |
|---|---|
| NOV24g, | MLRRVTVAAVCATRRKLCEAGRELAALWGIETRGRCEDSAAARPFPILGENSRVARLGADESEEEGR |
| CG56149-06 | |
| Protein Sequence | RGSLSNAGDPEIVKSPSDPKQYRYIKLQNGLQALLISDLVNWFKAHRGPGSKMLSVHVVGYGKYELE |
| | EDGTPSSEDSNSSCEVMQLTYLPTSPLLADCIIPITDIRAFTTTLNLLPYHKIVK |

| | |
|---|---|
| SEQ ID NO:213 | 3479 bp |

| | |
|---|---|
| NOV24h, | CCACCATGCTGAGGAGAGTCACTGTTGCTGCAGTCTGTGCCACCCGGAGGAAGTTGTGTGAGGCCGG |
| CG56149-08 | |
| DNA Sequence | GCGGGAGCTCGCGGCGCTCTGGGGAATCGAAACGCGGGGTCGGTGCGAAGACTCTGCTGCTGCCAGA |
| | CCCTTTCCTATTCTGGCCATGCCTGGAAGGAACAAGGCGAAGTCTACCTGCAGCTGCCCTGACCTGC |
| | AGCCCAATGGACAGGATCTGGGCGAGAACAGCCGGGTTGCCCGTCTAGGAGCGGATGAATCTGAGGA |
| | AGAGGGACGGAGGGGGTCTCTCAGTAATGCTGGGGACCCTGAGATCGTCAAGTCTCCCAGCGACCCC |
| | AAGCAATACCGATACATCAAATTACAGAATGGCTTGCAGGCACTTCTGATTTCAGACCTAAGTAATA |
| | TGGAAGGTAAAACAGGAAATACAACAGATGATGAAGAAGAAGAGGAGGTGGAGGAAGAAGAAGAAGA |
| | TGATGATGAAGATTCTGGAGCTGAAATAGAAGATGACGATGAAGAGGGTTTTGATGATGAAGATGAG |
| | TTTGATGATGAACATGATGATGATCTTGATACTGAGGATAATGAATTGGAAGAATTAGAAGAGAGAG |
| | CAGAAGCTAGAAAAAAAACTACTGAAAAACAGTCTGCAGCGGCTCTTTGTGTTGGAGTTGGGAGTTT |
| | CGCTGATCCAGATGACCTGCCGGGGCTGGCACACTTTTTGGAGCACATGGTATTCATGGGTAGTTTG |
| | AAATATCCAGATGAGAATGGATTTGATGCCTTCCTGAAGAAGCATGGGGGTAGTGATAATGCCTCAA |
| | CTGATTGTGAACGCACTGTCTTTCAGTTTGATGTCCAGAGGAAGTACTTCAAGGAAGCTCTTGATAG |
| | ATGGGCGCAGTTCTTCATCCACCCACTAATGATCAGAGATGCAATTGACCGTGAAGTTGAAGCTGTT |
| | GATAGTGAATATCAACTTGCAAGGCCTTCTGATGCAAACAGAAAGGAAATGTTGTTTGGAAGCCTTG |
| | CTAGACCTGGACATCCTATGGGAAAATTTTTTTGGGGAAATGCTGAGACGCTCAAGCATGAGCCAAG |
| | AAAGAATAATATTGATACACATGCTAGATTGAGAGAATTCTGGATGCGTTACTACTCTTCTCATTAC |
| | ATGACTTTAGTGGTTCAATCCAAAGAAACACTGGATACTTTGGAAAAGTGGGTGACTGAAATCTTCT |
| | CTCAGATACCAAACAATGGGTTACCCAGACCAAACTTTGGCCATTTAACGGATCCATTTGACACACC |
| | AGCATTTAACAAACTTTATAGAGTTGTTCCAATCAGAAAAATTCATGCTCTGACCATCACATGGGCA |
| | CTTCCTCCTCAACAGCAACATTACAGGGTGAAGCCACTTCATTATATATCCTGGCTGGTTGGACATG |
| | AAGGCAAAGGCAGCATTCTTTCTTTCCTTAGGAAAAAATGCTGGGCTCTTGCACTGTTTGGTGGAAA |
| | TGGTGAGACAGGATTTGAGCAAAATTCTACTTATTCAGTGTTCAGCATTTCTATTACATTGACTGAT |
| | GAGGGTTATGAACATTTTTATGAGGTTGCTTACACTGTCTTTCAGTATTTAAAAATGCTGCAGAAGC |
| | TAGGCCCAAAAAAAAGAATTTTTGAAGAGATTCGGAAAATTGAGGATAATGAATTTCATTACCAAGA |
| | ACAGACAGATCCAGTTGAGTATGTGGAAAACATGTGTGAGAACATGCAGCTGTACCCATTGCAGGAC |
| | ATTCTCACTGGAGATCAGCTTCTTTTTGAATACAAGCCAGAAGTCATTGGTGAAGCCTTGAATCAGC |
| | TAGTTCCTCAAAAAGCAAATCTTGTTTTACTGTCTGGTGCTAATGAGGGAAAATGTGACCTCAAGGA |
| | GAAATGGTTTGGAACTCAATATAGTATAGAAGATATTGAAAACTCTTGGGCTGAACTGTGGAATAGT |
| | AATTTCGAATTAAATCCAGATCTTCATCTTCCAGCTGAAAACAAGTACATAGCCACGGACTTTACGT |
| | TGAAGGCTTTCGATTGCCCGGAAACAGAATACCCAGTTAAAATTGTGAATACTCCACAAGGTTGCCT |
| | GTGGTATAAGAAAGACAACAAATTCAAAATCCCCAAAGCATATATACGTTTCCATCTAATTTCACCG |
| | TTGATACAGAAATCTGCAGCAAATGTGGTCCTCTTTGATATCTTTGTCAATATCCTTACGCATAACC |
| | TTGCGGAACCAGCTTATGAAGCAGATGTGGCACAGCTGGAGTATAAACTGGTAGCTGGAGAACATGG |
| | TTTAATTATTCGAGTGAAAGGATTTAACCACAAACTACCTCTACTGTTTCAGCTCATTATTGACTAC |
| | TTAGCTGAGTTCAATTCCACACCAGCTGTCTTTACAATGATAACTGAGCAGTTGAAGAAGACCTACT |
| | TTAACATCCTCATCAAGCCCGAGACTTTGGCCAAAGATGTACGGCTTTTAATCCTGGAATATGCCCG |
| | TTGGTCTATGATTGACAAGTACCAGGCTTTGATGGACGGCCTTTCCCTTGAGTCTCTGCTGAGCTTC |
| | GTCAAAGAATTCAAATCCCAGCTCTTTGTGGAGGGCCTGGTACAAGGGAATGTCACAAGCACAGAAT |
| | CTATGGATTTCCTGAAATATGTTGTTGACAAACTAAACTTCAAGCCTCTGGAGCAGGAGATGCCTGT |
| | GCAGTTCCAGGTGGTAGAGCTGCCCAGTGGCCACCATCTATGCAAAGTGAAAGCTCTGAACAAGGGT |
| | GATGCCAACTCTGAAGTCACTGTGTACTACCAGTCAGGTACCAGGAGTCTAAGAGAATATACGCTTA |
| | TGGAGCTGCTTGTGATGCACATGGAAGAACCTTGTTTTGACTTCCTTCGAACCAAGCAGACCCTTGG |
| | GTACCATGTCTACCCTACCTGTAGGAACACATCCGGGATTCTAGGATTTTCTGTCACTGTGGGGACT |
| | CAGGCAACCAAATACAATTCTGAAGTTGTTGATAAGAAGATAGAAGAGTTTCTTTCTAGCTTTGAGG |
| | AGAAGATTGAGAACCTCACTGAAGAGGCATTCAACACCCAGGTCACAGCTCTCATCAAGCTGAAGGA |
| | GTGTGAGGATACCCACCTTGGGGAGGAGGTGGATAGGAACTGGAATGAAGTGGTTACACAGCAGTAC |
| | CTCTTTGACCGCCTTGCCCACGAGATTGAAGCACTGAAGTCATTCTCAAAATCAGACCTGGTCAACT |
| | GGTTCAAGGCCCATAGAGGGCCAGGAAGTAAAATGCTCAGCGTTCATGTTGTTGGATATGGGAAGTA |
| | TGAACTGGAAGAGGATGGTACCCCTTCTAGTGAGGATTCAAATTCTTCTTGTGAAGTGATGCAGCTG |
| | ACCTACCTGCCAACCTCTCCTCTGCTGGCAGATTGTATCATCCCCATTACTGATATCAGGGCTTTCA |
| | CAACAACACTCAACCTTCTCCCCTACCATAAAATAGTCAAACACCATCACCACCATCACTAA |

| | | |
|---|---|---|
| ORF Start: ATG at 6 | | ORF Stop: at 3459 |
| SEQ ID NO: 214 | 1151 aa | MW at 131698.4 kD |

| | |
|---|---|
| NOV24h, | MLRRVTVAAVCATRRKLCEAGRELAALWGIETRGTCEDSAAARPFPILAMPGRNKAKSTCSCPDLQP |
| CG56149-08 | |
| Protein Sequence | NGQDLGENSRVARLGADESEEEGRRGSLSNAGDPEIVKSPSDPKQYRYIKLQNGLQALLISDLSNME |
| | GKTGNTTDDEEEEEVEEEEEDDDEDSGAEIEDDDEEGFDDEDEFDDEHDDDLDTEDNELEELEERAE |
| | ARKKTTEKQSAAALCVGVGSFADPDDLPGLAHFLEHMVFMGSLKYPDENGFDAFLKKHGGSDNASTD |
| | CERTVFQFDVQRKYFKEALDRWAQFFIHPLMIRDAIDREVEAVDSEYQLARPSDANRKEMLFGSLAR |
| | PGHPMGKFFWGNAETLKHEPRKNNIDTHARLREFWMRYYSSHYMTLVVQSKETLDTLEKWVTEIFSQ |
| | IPNNGLPRPNFGHLTDPFDTPAFNKLYRVVPIRKIHALTITWALPPQQQHYRVKPLHYISWLVGHEG |
| | KGSILSFLRKKCWALALFGGNGETGFEQNSTYSVFSISITLTDEGYEHFYEVAYTVFQYLKMLQKLG |
| | PKKRIFEEIFKIEDNEFHYQEQTDPVEYVENMCENMQLYPLQDILTGDQLLFEYKPEVIGEALNQLV |
| | PQKANLVLLSGANEGKCDLKEKWFGTQYSIEDIENSWAELWNSNFELNPDLHLPAENKYIATDFTLK |
| | AFDCPETEYPVKIVNTPQGCLWYKKDNKFKIPKAYIRFHLISPLIQKSAANVVLFDIFVNILTHNLA |
| | EPAYEADVAQLEYKLVAGEHGLIIRVKGFNHKLPLLFQLIIDYLAEFNSTPAVFTMITEQLKKTYFN |
| | ILIKPETLAKDVRLLILEYARWSMIDKYQALMDGLSLESLLSFVKEFKSQLFVEGLVQGNVTSTESM |
| | DFLKYVVDKLNFKPLEQEMPVQFQVVELPSGHHLCKVKALNKGDANSEVTVYYQSGTRSLREYTLME |
| | LLVMHMEEPCFDFLRTKQTLGYHVYPTCRNTSGILGFSVTVGTQATKYNSEVVDKKIEEFLSSFEEK |
| | IENLTEEAFNTQVTALIKLKECEDTHLGEEVDRNWNEVVTQQYLFDRLAHEIEALKSFSKSDLVNWF |
| | KAHRGPGSKMLSVHVVGYGKYELEEDGTPSSEDSNSSCEVMQLTYLPTSPLLADCIIPITDIRAFTT |
| | TLNLLPYHKIVK |

Sequence comparison of the above protein sequences yields the following sequence relationships shown in Table 24B.

TABLE 24B
Comparison of NOV24a against NOV24b through NOV24h.

| | | Identities/ |
|---|---|---|
| | NOV24a Residues/ | Similarities for the |
| Protein Sequence | Match Residues | Matched Region |
| NOV24b | 10 . . . 1159 | 1143/1150 (99%) |
| | 2 . . . 1151 | 1148/1150 (99%) |
| NOV24c | 10 . . . 1159 | 1123/1218 (92%) |
| | 2 . . . 1219 | 1136/1218 (93%) |
| NOV24d | 10 . . . 1159 | 1148/1155 (99%) |
| | 2 . . . 1156 | 1150/1155 (99%) |
| NOV24e | 10 . . . 1159 | 1149/1150 (99%) |
| | 2 . . . 1151 | 1150/1150 (99%) |
| NOV24f | 219 . . . 345 | 127/127 (100%) |
| | 1 . . . 127 | 127/127 (100%) |
| NOV24g | 10 . . . 140 | 106/131 (80%) |
| | 2 . . . 108 | 106/131 (80%) |
| NOV24h | 10 . . . 1159 | 1149/1150 (99%) |
| | 2 . . . 1151 | 1150/1150 (99%) |

Further analysis of the NOV24a protein yielded the following properties shown in Table 24C.

TABLE 24C
Protein Sequence Properties NOV24a

| SignalP analysis: | No Known Signal Sequence Predicted | | | |
|---|---|---|---|---|
| PSORT II analysis: | PSG: a new signal peptide prediction method | | | |
| | N-region: length 11; pos.chg 1; neg.chg 0 | | | |
| | H-region: length 0; peak value 1.00 | | | |
| | PSG score: −3.40 | | | |
| | GvE: von Heijne's method for signal seq. | | | |
| | recognition | | | |
| | GvH score (threshold: −2.1): −6.59 | | | |
| | possible cleavage site: between 42 and 43 | | | |
| | >>>Seems to have no N-terminal signal peptide | | | |
| | ALOM: Klein et al's method for TM region | | | |
| | allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 1.96 (at 726) | | | |
| | ALOM score: 1.96 (number of TMSs: 0) | | | |
| | MITDISC: discrimination of mitochondrial | | | |
| | targeting seq | | | |
| | R content: | 4 | Hyd Moment(75): | 2.28 |
| | Hyd Moment(95): | 3.41 | G content: | 1 |
| | D/E content: | 1 | S/T content: | 3 |
| | Score: | −1.76 | | |

| Gavel: prediction of cleavage sites for | | |
|---|---|---|
| mitochondrial preseq | | |
| R-2 motif at 33 RRK\|LC | | |
| NUCDISC: discrimination of nuclear | | |
| localization signals | | |
| pat4: PKKR (4) at 545 | | |
| pat7: PKKRIFE (5) at 545 | | |
| bipartite: none | | |
| content of basic residues: 10.6% | | |
| NLS Score: 0.21 | | |
| KDEL: ER retention motif in the | | |
| C-terminus: none | | |
| ER Membrane Retention Signals: | | |
| KKXX-like motif in the C-terminus: HKIV | | |
| SKL: peroxisomal targeting signal in the | | |
| C-terminus: none | | |
| PTS2: 2nd peroxisomal targeting signal: none | | |
| VAC: possible vacuolar targeting motif: none | | |
| RNA-binding motif: none | | |
| Actinin-type actin-binding motif: | | |
| type 1: none | | |
| type 2: none | | |
| NMYR: N-myristoylation pattern none | | |
| Prenylation motif: none | | |
| memYQRL: transport motif from cell surface | | |
| to Golgi: none | | |
| Tyrosines in the tail: none | | |
| Dileucine motif in the tail: none | | |
| checking 63 PROSITE DNA binding motifs: none | | |
| checking 71 PROSITE ribosomal protein | | |
| motifs: none | | |
| checking 33 PROSITE prokaryotic DNA | | |
| binding motifs: none | | |
| NNCN: Reinhardt's method for Cytoplasmic/ | | |
| Nuclear discrimination | | |
| Prediction: cytoplasmic | | |
| Reliability: 89 | | |
| COIL: Lupas's algorithm to detect | | |
| coiled-coil regions | | |
| 186 | D | 0.54 |
| 187 | D | 0.87 |
| 188 | E | 0.91 |
| 189 | H | 0.91 |
| 190 | D | 0.91 |
| 191 | D | 0.96 |
| 192 | D | 0.96 |
| 193 | L | 0.96 |
| 194 | D | 0.96 |
| 195 | T | 0.96 |
| 196 | E | 0.96 |
| 197 | D | 0.96 |
| 198 | N | 0.96 |
| 199 | E | 0.96 |
| 200 | L | 0.96 |
| 201 | E | 0.96 |
| 202 | E | 0.96 |
| 203 | L | 0.96 |
| 204 | E | 0.96 |
| 205 | E | 0.96 |
| 206 | R | 0.96 |
| 207 | A | 0.96 |
| 208 | E | 0.96 |
| 209 | A | 0.96 |
| 210 | R | 0.96 |
| 211 | K | 0.96 |
| 212 | K | 0.96 |
| 213 | T | 0.96 |
| 214 | T | 0.96 |
| 215 | E | 0.96 |
| 216 | K | 0.96 |
| 217 | Q | 0.96 |
| 218 | S | 0.96 |
| 219 | A | 0.94 |
| 220 | A | 0.88 |
| 221 | A | 0.83 |
| 222 | L | 0.82 |

total: 37 residues
Final Results (k = 9/23):
47.8%: mitochondrial
39.1%: cytoplasmic
13.0%: nuclear
>>prediction for CG56149-07 is mit (k = 23)

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAU28119 | Novel human secretory | 10 . . . 1159 | 1148/1150 (99%) | 0.0 |
| | protein, Seq ID No 288- | 2 . . . 1151 | 1150/1150 (99%) | |
| | Homo sapiens, 1151 aa. | | | |
| | [WO200166689-A2, | | | |
| | 13-SEP-2001] | | | |
| AAW95039 | Human N-arginine dibasic | 10 . . . 1159 | 1146/1150 (99%) | 0.0 |
| | (h-NRD) convertase - Homo | 2 . . . 1151 | 1149/1150 (99%) | |
| | sapiens, 1151 aa. | | | |
| | [WO9902664-A1, | | | |
| | 21-JAN-1999] | | | |
| AAU28120 | Novel human secretory | 10 . . . 1159 | 1128/1218 (92%) | 0.0 |
| | protein, Seq ID No 289 - | 2 . . . 1219 | 1138/1218 (92%) | |
| | Homo sapiens, 1219 aa. | | | |
| | [WO200166689-A2, | | | |
| | 13-SEP-2001] | | | |
| AAU28308 | Novel human secretory | 10 . . . 1159 | 1051/1176 (89%) | 0.0 |
| | protein, Seq ID No 665- | 2 . . . 1177 | 1070/1176 (90%) | |
| | Homo sapiens, 1177 aa. | | | |
| | [WO200166689-A2, | | | |
| | 13-SEP-2001] | | | |
| AAU28307 | Novel human secretory | 10 . . . 1159 | 1051/1176 (89%) | 0.0 |
| | protein, Seq ID No 664 - | 2 . . . 1177 | 1070/1176 (90%) | |
| | Homo sapiens, 1177 aa. | | | |
| | [WO20016668 9-A2, | | | |
| | 13-SEP-2001] | | | |

In a BLAST search of public sequence datbases, the NOV24a protein was found to have homology to the proteins shown in the BLASTP data in Table 24E.

TABLE 24E
Public BLASTP Results for NOV24a

| Protein | | Residues/ | Identities/ | |
|---|---|---|---|---|
| Accession | | Match | Similarities for the | Expect |
| Number | Protein/Organism/Length | Residues | Matched Portion | Value |
| Q96HB2 | Nardilysin (N-arginine | 10 . . . 1159 | 1147/1150 (99%) | 0.0 |
| | dibasic convertase) | 2 . . . 1150 | 1149/1150 (99%) | |
| | (DJ657D16.1) - Homo | | | |
| | sapiens (Human), 1150 aa. | | | |
| O15241 | NRD1 convertase (EC | 10 . . . 1159 | 1143/1150 (99%) | 0.0 |
| | 3.4.24.61) - Homo sapiens | 2 . . . l151 | 1148/1150 (99%) | |
| | (Human), 1151 aa. | | | |
| O43847 | Nardilysin precursor (EC | 10 . . . 1139 | 1120/1130 (99%) | 0.0 |
| | 3.4.24.61) (N-arginine | 2 . . . 1130 | 1123/1130 (99%) | |
| | dibasic convertase) (NRD | | | |
| | convertase) - Homo sapiens | | | |
| | (Human), 1147 aa. | | | |
| AAH36128 | Hypothetical protein | 10 . . . 1159 | 1071/1165 (91%) | 0.0 |
| | MGC25477 - Mus musculus | 2 . . . 1161 | 1106/1165 (94%) | |
| | (Mouse), 1161 aa. | | | |
| Q8R320 | Similar to N-arginine dibasic | 10 . . . 1159 | 1070/1165 (91%) | 0.0 |
| | convertase 1 - Mus musculus | 2 . . . 1161 | 1106/1165 (94%) | |
| | (Mouse), 1161 aa. | | | |

PFam analysis predicts that the NOV24a protein contains the domains shown in the Table 24F.

TABLE 24F
Domain Analysis of NOV24a

| | | Identities/ | |
|---|---|---|---|
| | NOV24a | Similarities | |
| | Match | for the Matched | Expect |
| Pfam Domain | Region | Region | Value |
| Peptidase_M16 | 206 . . . 345 | 58/161 (36%) | 1.1e−59 |
| | | 125/161 (78%) | |

### Example 25

The NOV25 clone was analyzed, and the nucleotide and encoded polypeptide sequences are shown in Table 25A.

TABLE 25A
NOV25 Sequence Analysis

| | |
|---|---|
| SEQ ID NO:215 | 3147bp |

| | |
|---|---|
| NOV25a, | GCATGGAGGCGGCGCATCTGCTCCCGGCCGCCGACGTGCTGCGCCACTTCTCGGTGACAGCCGAGGG |
| CG56216-01 | |
| DNA Sequence | CGGCCTGAGCCCGGCGCAGGTGACCGGCGCGCGGGAGCGCTACGGCCCCAACGAGCTCCCGAGTGAG |
| | GAAGGGAAGTCCCTGTGGGAGCTGGTGCTGGAACAGTTTGAGGACCTCCTGGTGCGCATCCTGCTGC |
| | TGGCTGCCCTTGTCTCCTTTGTCCTGGCCTGGTTCGAGGAGGGCGAGGAGACCACGACCGCCTTCGT |
| | GGAGCCCCTGGTCATCATGCTGATCCTCGTGGCCAACGCCATTGTGGGCGTGTGGCAGGAACGCAAC |
| | GCCGAGAGTGCCATCGAGGCCCTGAAGGAGTATGAGCCTGAGATGGGCAAGGTGATCCGCTCGGACC |
| | GCAAGGGCGTGCAGAGGATCCGTGCCCGGGACATCGTCCCAGGGGACATTGTAGAAGTGGCAGTGGG |
| | GGACAAAGTGCCTGCTGACCTCCGCCTCATCGAGATCAAGTCCACCACGCTGCGAGTGGACCAGTCC |
| | ATCCTGACGGGTGAATCTGTGTCCGTGACCAAGCACACAGAGGCCATCCCAGACCCCAGAGCTGTGA |
| | ACCAGGACAAGAAGAACATGCTGTTTTCTGGCACCAATATCACATCGGGCAAAGCGGTGGGTGTGGC |
| | CGTGGCCACCGGCCTGCACACGGAGCTGGGCAAGATCCGGAGCCAGATGGCGGCAGTCGAGCCCGAG |
| | CGGACGCCGCTGCAGCGCAAGCTGGACGAGTTTGGACGGCAGCTGTCCCACGCCATCTCTGTGATCT |
| | GTGTGGCCGTGTGGGTCATCAACATCGGCCACTTCGCCGACCCGGCCCACGGTGGCTCCTGGCTGCG |
| | TGGCGCTGTCTACTACTTCAAGATCGCCGTGGCCCTGGCGGTGGCGGCCATCCCCGAGGGCCTCCCG |
| | GCTGTCATCACTACATGCCTGGCACTGGGCACGCGGCGCATGGCACGCAAGAACGCCATCGTGCGAA |
| | GCCTGCCGTCCGTGGAGACCCTGGGCTGCACCTCAGTCATCTGCTCCGACAAGACGGGCACGCTCAC |
| | CACCAATCAGATGTCTGTCTGCCGGATGTTCGTGGTAGCCGAGGCCGATGCGGGCTCCTGCCTTTTG |
| | CACGAGTTCACCATCTCGGGTACCACGTATACCCCCGAGGGCGAAGTGCGGCAGGGGGATCAGCCTG |
| | TGCGCTGCGGCCAGTTCGACGGGCTGGTGGAGCTGGCGACCATCTGCGCCCTGTGCAACGACTCGGC |
| | TCTGGACTACAACGAGGCCAAGGGTGTGTATGAGAAGGTGGGAGAGGCCACGGAGACAGCTCTGACT |
| | TGCCTGGTGGAGAAGATGAACGTGTTCGACACCGACCTGCAGGCTCTGTCCCGGGTGGAGCGAGCTG |
| | GCGCCTGTAACACGGTCATCAAGCAGCTGATGCGGAAGGAGTTCACCCTGGAGTTCTCCCGAGACCG |
| | GAAATCCATGTCCGTGTACTGCACGCCCACCCGCCCTCACCCTACTGGCCAGGGCAGCAAGATGTTT |
| | GTGAAGGGGGCTCCTGAGAGTGTGATCGAGCGCTGTAGCTCAGTCCGCGTGGGGAGCCGCACAGCAC |
| | CCCTGACCCCCACCTCCAGGGAGCAGATCCTGGCAAAGATCCGGGATTGGGGCTCAGGCTCAGACAC |
| | GCTGCGCTGCCTGGCACTGGCCACCCGGGACGCGCCCCCAAGGAAGGAGGACATGGAGCTGGACGAC |
| | TGCGGCAAGTTTGTGCAGTACGAGACGGACCTGACCTTCGTGGGCTGCGTAGGCATGCTGGACCCGC |
| | CGCGACCCGAGGTGGCTGCCTGCATCACACGCTGCTACCAGGCGGGCATCCGCGTGGTCATGATCAC |
| | GGGGGATAACAAAGGCACTGCCGTGGCCATCTGCCGCAGGCTTGGCATCTTTGGGGACACGGAAGAC |
| | GTGGCGGGCAAGGCCTACACGGGCCGCGAGTTTGATGACCTCAGCCCCGAGCAGCAGCGCCAGGCCT |
| | GCCGCACCGCCCGCTGCTTCGCCCGCGTGGAGCCCGCACACAAGTCCCGCATCGTGGAGAACCTGCA |
| | GTCCTTTAACGAGATCACTGCTATGACTGGTGATGGAGTGAACGACGCACCAGCCCTGAAGAAAGCA |
| | GAGATCGGCATCGCCATGGGCTCAGGCACGGCCGTGGCCAAGTCGGCGGCAGAGATGGTGCTGTCAG |
| | ATGACAACTTTGCCTCCATCGTGGCTGCGGTGGAGGAGGGCCGGGCCATCTACAGCAACATGAAGCA |
| | ATTCATCCGCTACCTCATCTCCTCCAATGTTGGCGAGGTCGTCTGCATCTTCCTCACGGCAATTCTG |
| | GGCCTGCCCGAAGCCCTGATCCCTGTGCAGCTGCTCTGGGTGAACCTGGTGACAGACGGCCTACCTG |
| | CCACGGCTCTGGGCTTCAACCCGCCAGACCTGGACATCATGGAGAAGCTGCCCCGGAGCCCCCGAGA |
| | AGCCCTCATCAGTGGCTGGCTCTTCTTCCGATACCTGGCTATCGGAGTGTACGTAGGCCTGGCCACA |
| | GTGGCTGCCGCCACCTGGTGGTTTGTGTATGACGCCGAGGGACCTCACATCAACTTCTACCAGCTGA |
| | GGAACTTCCTGAAGTGCTCCGAAGACAACCCGCTCTTTGCCGGCATCGACTGTGAGGTGTTCGAGTC |
| | ACGCTTCCCCACCACCATGGCCTTGTCCGTGCTCGTGACCATTGAAATGTGCAATGCCCTCAACAGC |
| | GTCTCGGAGAACCAGTCGCTGCTGCGGATGCCGCCCTGGATGAACCCCTGGCTGCTGGTGGCTGTGG |
| | CCATGTCCATGGCCCTGCACTTCCTCATCCTGCTCGTGCCGCCCCTGCCTCTCATTTTCCAGGTGAC |
| | CCCACTGAGCGGGCGCCAGTGGGTGGTGGTGCTCCAGATATCTCTGCCTGTCATCCTGCTGGATGAG |
| | GCCCTCAAGTACCTGTCCCGGAACCACATGCACGAAGAAATGAGCCAGAAGTGAGCGCTGGGAACAG |
| | AGTGGAGTCTCCGGTGTGTACCTCAGACTGATGGTGCCCATGTGTTCGCCTCCGCCCCCCACCCTTG |
| | CCACCACACTCGCCCACTTGCCCACCGGGTCCCGCCGGATAAATGACAGGCCCGAGGTCAGAATG |

| | | |
|---|---|---|
| ORF Start: ATG at 3 | | ORF Stop: TGA at 3000 |
| SEQ ID NO: 216 | 999 aa | MW at 109224.8 kD |

| | |
|---|---|
| NOV25a, | MEAAHLLPAADVLRHFSVTAEGGLSPAQVTGARERYGPNELPSEEGKSLWELVLEQFEDLLVRILLL |
| CG56216-01 | |
| Protein Sequence | AALVSFVLAWFEEGEETTTAFVEPLVIMLILVANAIVGVWQERNAESAIEALKEYEPEMGKVIRSDR |
| | KGVQRIRARDIVPGDIVEVAVGDKVPADLRLIEIKSTTLRVDQSILTGESVSVTKHTEAIPDPRAVN |
| | QDKKNMLFSGTNITSGKAVGVAVATGLHTELGKIRSQMAAVEPERTPLQRKLDEFGRQLSHAISVIC |
| | VAVWVINIGHFADPAHGGSWLRGAVYYFKIAVALAVAAIPEGLPAVITTCLALGTRRMARKNAIVRS |
| | LPSVETLGCTSVICSDKTGTLTTNQMSVCRMFVVAEADAGSCLLHEFTISGTTYTPEGEVRQGDQPV |
| | RCGQFDGLVELATICALCNDSALDYNEAKGVYEKVGEATETALTCLVEKMNVFDTDLQALSRVERAG |
| | ACNTVIKQLMRKEFTLEFSRDRKSMSVYCTPTRPHPTGQGSKMFVKGAPESVIERCSSVRVGSRTAP |
| | LTPTSREQILAKIRDWGSGSDTLRCLALATRDAPPRKEDMELDDCGKFVQYETDLTFVGCVGMLDPP |
| | RPEVAACITRCYQAGIRVVMITGDNKGTAVAICRRLGIFGDTEDVAGKAYTGREFDDLSPEQQRQAC |
| | RTARCFARVEPAHKSRIVENLQSFNEITAMTGDGVNDAPALKKAEIGIAMGSGTAVAKSAAEMVLSD |
| | DNFASIVAAVEEGRAIYSNMKQFIRYLISSNVGEVVCIFLTAILGLPEALIPVQLLWVNLVTDGLPA |
| | TALGFNPPDLDIMEKLPRSPREALISGWLFFRYLAIGVYVGLATVAAATWWFVYDAEGPHINFYQLR |
| | NFLKCSEDNPLFAGIDCEVFESRFPTTMALSVLVTIEMCNALNSVSENQSLLRMPPWMNPWLLVAVA |
| | MSMALHFLILLVPPLPLIFQVTPLSGRQWVVVLQISLPVILLDEALKYLSRNHMHEEMSQK |

| | |
|---|---|
| SEQ ID NO: 217 | 3005 bp |

| | |
|---|---|
| NOV25b, | CCACCATGGAGGCGGCGCATCTGCTCCCGGCCGCCGACGTGCTGCGCCACTTCTCGGTGACAGCCGA |
| 222682222 DNA | |
| Sequence | GGGCGGCCTGAGCCCGGCGCAGGTGACCGGCGCGCGGGAGCGCTACGGCCCCAACGAGCTCCCGAGT |
| | GAGGAAGGGAAGTCCCTGTGGGAGCTGGTGCTGGAACAGTTTGAGGACCTCCTGGTGCGCATCCTGC |
| | TGCTGGCTGCCCTTGTCTCCTTTGTCCTGGCCTGGTTCGAGGAGGGCGAGGAGACCACGACCGCCTT |
| | CGTGGAGCCCCTGGTCATCATGCTGATCCTCGTGGCCAACGCCATTGTGGGCGTGTGGCAGGAACGC |
| | AACGCCGAGAGTGCCATCGAGGCCCTGAAGGAGTATGAGCCTGAGATGGGCAAGGTGATCCGCTCGG |
| | ACCGCAAGGGCGTGCAGAGGATCCGTGCCCGGGACATCGTCCCAGGGGACATTGTAGAAGTGGCAGT |
| | GGGGGACAAAGTGCCTGCTGACCTCCGCCTCATCGAGATCAAGTCCACCACGCTGCGAGTGGACCAG |
| | TCCATCCTGACGGGTGAATCTGTGTCCGTGACCAAGCACACAGAGGCCATCCCAGACCCCAGAGCTG |
| | TGAACCAGGACAAGAAGAACATGCTGTTTTCTGGCACCAATATCACATCGGGCAAAGCGGTGGGTGT |
| | GGCCGTGGCCACCGGCCTGCACACGGAGCTGGGCAAGATCCGGAGCCAGATGGCGGCAGTCGAGCCC |
| | GAGCGGACGCCGCTGCAGCGCAAGCTGGACGAGTTTGGACGGCAGCTGTCCCACGCCATCTCTGTGA |
| | TCTGTGTGGCCGTGTGGGTCATCAACATCGGCCACTTCGCCGACCCGGCCCACGGTGGCTCCTGGCT |
| | GCGTGGCGCTGTCTACTACTTCAAGATCGCCGTGGCCCTGGCGGTGGCGGCCATCCCCGAGGGCCTC |
| | CCGGCTGTCATCACTACATGCCTGGCACTGGGCACGCGGCGCATGGCACGCAAGAACGCCATCGTGC |
| | GAAGCCTGCCGTCCGTGGAGACCCTGGGCTGCACCTCAGTCATCTGCTCCGACAAGACGGGCACGCT |
| | CACCACCAATCAGATGTCTGTCTGCCGGATGTTCGTGGTAGCCGAGGCCGATGCGGGCTCCTGCCTT |
| | TTGCACGAGTTCACCATCTCGGGTACCACGTATACCCCCGAGGGCGAAGTGCGGCAGGGGGATCAGC |
| | CTGTGCGCTGCGGCCAGTTCGACGGGCTGGTGGAGCTGGCGACCATCTGCGCCCTGTGCAACGACTC |
| | GGCTCTGGACTACAACGAGGCCAAGGGTGTGTATGAGAAGGTGGGAGAGGCCACGGAGACAGCTCTG |
| | ACTTGCCTGGTGGAGAAGATGAACGTGTTCGACACCGACCTGCAGGCTCTGTCCCGGGTGGAGCGAG |
| | CTGGCGCCTGTAACACGGTCATCAAGCAGCTGATGCGGAAGGAGTTCACCCTGGAGTTCTCCCGAGA |
| | CCGGAAATCCATGTCCGTGTACTGCACGCCCACCCGCCCTCACCCTACTGGCCAGGGCAGCAAGATG |
| | TTTGTGAAGGGGGCTCCTGAGAGTGTGATCGAGCGCTGTAGCTCAGTCCGCGTGGGGAGCCGCACAG |
| | CACCCCTGACCCCCACCTCCAGGGAGCAGATCCTGGCAAAGATCCGGGATTGGGGCTCAGGCTCAGA |
| | CACGCTGCGCTGCCTGGCACTGGCCACCCGGGACGCGCCCCCAAGGAAGGAGGACATGGAGCTGGAC |
| | GACTGCGGCAAGTTTGTGCAGTACGAGACGGACCTGACCTTCGTGGGCTGCGTAGGCATGCTGGACC |
| | CGCCGCGACCCGAGGTGGCTGCCTGCATCACACGCTGCTACCAGGCGGGCATCCGCGTGGTCATGAT |
| | CACGGGGGATAACAAAGGCACTGCCGTGGCCATCTGCCGCAGGCTTGGCATCTTTGGGGACACGGAA |
| | GACGTGGCGGGCAAGGCCTACACGGGCCGCGAGTTTGATGACCTCAGCCCCGAGCAGCAGCGCCAGG |
| | CCTGCCGCACCGCCCGCTGCTTCGCCCGCGTGGAGCCCGCACACAAGTCCCGCATCGTGGAGAACCT |
| | GCAGTCCTTTAACGAGATCACTGCTATGACTGGTGATGGAGTGAACGACGCACCAGCCCTGAAGAAA |
| | GCAGAGATCGGCATCGCCATGGGCTCAGGCACGGCCGTGGCCAAGTCGGCGGCAGAGATGGTGCTGT |
| | CAGATGACAACTTTGCCTCCATCGTGGCTGCGGTGGAGGAGGGCCGGGCCATCTACAGCAACATGAA |
| | GCAATTCATCCGCTACCTCATCTCCTCCAATGTTGGCGAGGTCGTCTGCATCTTCCTCACGGCAATT |
| | CTGGGCCTGCCCGAAGCCCTGATCCCTGTGCAGCTGCTCTGGGTGAACCTGGTGACAGACGGCCTAC |
| | CTGCCACGGCTCTGGGCTTCAACCCGCCAGACCTGGACATCATGGAGAAGCTGCCCCGGAGCCCCCG |
| | AGAAGCCCTCATCAGTGGCTGGCTCTTCTTCCGATACCTGGCTATCGGAGTGTACGTAGGCCTGGCC |
| | ACAGTGGCTGCCGCCACCTGGTGGTTTGTGTATGACGCCGAGGGACCTCACATCAACTTCTACCAGC |
| | TGAGGAACTTCCTGAAGTGCTCCGAAGACAACCCGCTCTTTGCCGGCATCGACTGTGAGGTGTTCGA |
| | GTCACGCTTCCCCACCACCATGGCCTTGTCCGTGCTCGTGACCATTGAAATGTGCAATGCCCTCAAC |
| | AGCGTCTCGGAGAACCAGTCGCTGCTGCGGATGCCGCCCTGGATGAACCCCTGGCTGCTGGTGGCTG |
| | TGGCCATGTCCATGGCCCTGCACTTCCTCATCCTGCTCGTGCCGCCCCTGCCTCTCATTTTCCAGGT |
| | GACCCCACTGAGCGGGCGCCAGTGGGTGGTGGTGCTCCAGATATCTCTGCCTGTCATCCTGCTGGAT |
| | GAGGCCCTCAAGTACCTGTCCCGGAACCACATGCACGAAGAAATGAGCCAGAAGTGA |

| | | |
|---|---|---|
| ORF Start: at 3 | | ORF Stop: TGA at 3003 |
| SEQ ID NO: 218 | 1000 aa | MW at 109325.9 kD |

| | |
|---|---|
| NOV25b, | TMEAAHLLPAADVLRHFSVTAEGGLSPAQVTGARERYGPNELPSEEGKSLWELVLEQFEDLLVRILL |
| 222682222 | |
| Protein Sequence | LAALVSFVLAWFEEGEETTTAFVEPLVIMLILVANAIVGVWQERNAESAIEALKEYEPEMGKVIRSD |
| | RKGVQRIRARDIVPGDIVEVAVGDKVPADLRLIEIKSTTLRVDQSILTGESVSVTKHTEAIPDPRAV |
| | NQDKKNMLFSGTNITSGKAVGVAVATGLHTELGKIRSQMAAVEPERTPLQRKLDEFGRQLSHAISVI |
| | CVAVWVINIGHFADPAHGGSWLRGAVYYFKIAVALAVAAIPEGLPAVITTCLALGTRRMARKNAIVR |
| | SLPSVETLGCTSVICSDKTGTLTTNQMSVCRMFVVAEADAGSCLLHEFTISGTTYTPEGEVRQGDQP |
| | VRCGQFDGLVELATICALCNDSALDYNEAKGVYEKVGEATETALTCLVEKMNVFDTDLQALSRVERA |
| | GACNTVIKQLMRKEFTLEFSRDRKSMSVYCTPTRPHPTGQGSKMFVKGAPESVIERCSSVRVGSRTA |
| | PLTPTSREQILAKIRDWGSGSDTLRCLALATRDAPPRKEDMELDDCGKFVQYETDLTFVGCVGMLDP |
| | PRPEVAACITRCYQAGIRVVMITGDNKGTAVAICRRLGIFGDTEDVAGKAYTGREFDDLSPEQQRQA |
| | CRTARCFARVEPAHKSRIVENLQSFNEITAMTGDGVNDAPALKKAEIGIAMGSGTAVAKSAAEMVLS |
| | DDNFASIVAAVEEGRAIYSNMKQFIRYLISSNVGEVVCIFLTAILGLPEALIPVQLLWVNLVTDGLP |
| | ATALGFNPPDLDIMEKLPRSPREALISGWLFFRYLAIGVYVGLATVAAATWWFVYDAEGPHINFYQL |
| | RNFLKCSEDNPLFAGIDCEVFESRFPTTMALSVLVTIEMCNALNSVSENQSLLRMPPWMNPWLLVAV |
| | AMSMALHFLILLVPPLPLIFQVTPLSGRQWVVVLQISLPVILLDEALKYLSRNHMHEEMSQK |

| | |
|---|---|
| SEQ ID NO: 219 | 3004 bp |

| | |
|---|---|
| NOV25c, | CACCATGGAGGCGGCGCATCTGCTCCCGGCCGCCGACGTGCTGCGCCACTTCTCGGTGACAGCCGAG |
| 248851003 DNA | |
| Sequence | GGCGGCCTGAGCCCGGCGCAGGTGACCGGCGCGCGGGAGCGCTACGGCCCCAACGAGCTCCCGAGTG |
| | AGGAAGGGAAGTCCCTGTGGGAGCTGGTGCTGGAACAGTTTGAGGACCTCCTGGTGCGCATCCTGCT |
| | GCTGGCTGCCCTTGTCTCCTTTGTCCTGGCCTGGTTCGAGGAGGGCGAGGAGACCACGACCGCCTTC |
| | GTGGAGCCCCTGGTCATCATGCTGATCCTCGTGGCCAACGCCATTGTGGGCGTGTGGCAGGAACGCA |
| | ACGCCGAGAGTGCCATCGAGGCCCTGAAGGAGTATGAGCCTGAGATGGGCAAGGTGATCCGCTCGGA |
| | CCGCAAGGGCGTGCAGAGGATCCGTGCCCGGGACATCGTCCCAGGGGACATTGTAGAAGTGGCAGTG |
| | GGGGACAAAGTGCCTGCTGACCTCCGCCTCATCGAGATCAAGTCCACCACGCTGCGAGTGGACCAGT |
| | CCATCCTGACGGGTGAATCTGTGTCCGTGACCAAGCACACAGAGGCCATCCCAGACCCCAGAGCTGT |
| | GAACCAGGACAAGAAGAACATGCTGTTTTCTGGCACCAATATCACATCGGGCAAAGCGGTGGGTGTG |
| | GCCGTGGCCACCGGCCTGCACACGGAGCTGGGCAAGATCCGGAGCCAGATGGCGGCAGTCGAGCCCG |
| | AGCGGACGCCGCTGCAGCGCAAGCTGGACGAGTTTGGACGGCAGCTGTCCCACGCCATCTCTGTGAT |
| | CTGTGTGGCCGTGTGGGTCATCAACATCGGCCACTTCGCCGACCCGGCCCACGGTGGCTCCTGGCTG |
| | CGTGGCGCTGTCTACTACTTCAAGATCGCCGTGGCCCTGGCGGTGGCGGCCATCCCCGAGGGCCTCC |
| | CGGCTGTCATCACTACATGCCTGGCACTGGGCACGCGGCGCATGGCACGCAAGAACGCCATCGTGCG |
| | AAGCCTGCCGTCCGTGGAGACCCTGGGCTGCACCTCAGTCATCTGCTCCGACAAGACGGGCACGCTC |
| | ACCACCAATCAGATGTCTGTCTGCCGGATGTTCGTGGTAGCCGAGGCCGATGCGGGCTCCTGCCTTT |
| | TGCACGAGTTCACCATCTCGGGTACCACGTATACCCCCGAGGGCGAAGTGCGGCAGGGGGATCAGCC |
| | TGTGCGCTGCGGCCAGTTCGACGGGCTGGTGGAGCTGGCGACCATCTGCGCCCTGTGCAACGACTCG |
| | GCTCTGGACTACAACGAGGCCAAGGGTGTGTATGAGAAGGTGGGAGAGGCCACGGAGACAGCTCTGA |
| | CTTGCCTGGTGGAGAAGATGAACGTGTTCGACACCGACCTGCAGGCTCTGTCCCGGGTGGAGCGAGC |
| | TGGCGCCTGTAACACGGTCATCAAGCAGCTGATGCGGAAGGAGTTCACCCTGGAGTTCTCCCGAGAC |
| | CGGAAATCCATGTCCGTGTACTGCACGCCCACCCGCCCTCACCCTACTGGCCAGGGCAGCAAGATGT |
| | TTGTGAAGGGGGCTCCTGAGAGTGTGATCGAGCGCTGTAGCTCAGTCCGCGTGGGGAGCCGCACAGC |
| | ACCCCTGACCCCCACCTCCAGGGAGCAGATCCTGGCAAAGATCCGGGATTGGGGCTCAGGCTCAGAC |
| | ACGCTGCGCTGCCTGGCACTGGCCACCCGGGACGCGCCCCCAAGGAAGGAGGACATGGAGCTGGACG |
| | ACTGCGGCAAGTTTGTGCAGTACGAGACGGACCTGACCTTCGTGGGCTGCGTAGGCATGCTGGACCC |
| | GCCGCGACCCGAGGTGGCTGCCTGCATCACACGCTGCTACCAGGCGGGCATCCGCGTGGTCATGATC |
| | ACGGGGGATAACAAAGGCACTGCCGTGGCCATCTGCCGCAGGCTTGGCATCTTTGGGGACACGGAAG |
| | ACGTGGCGGGCAAGGCCTACACGGGCCGCGAGTTTGATGACCTCAGCCCCGAGCAGCAGCGCCAGGC |
| | CTGCCGCACCGCCCGCTGCTTCGCCCGCGTGGAGCCCGCACACAAGTCCCGCATCGTGGAGAACCTG |
| | CAGTCCTTTAACGAGATCACTGCTATGACTGGTGATGGAGTGAACGACGCACCAGCCCTGAAGAAAG |
| | CAGAGATCGGCATCGCCATGGGCTCAGGCACGGCCGTGGCCAAGTCGGCGGCAGAGATGGTGCTGTC |
| | AGATGACAACTTTGCCTCCATCGTGGCTGCGGTGGAGGAGGGCCGGGCCATCTACAGCAACATGAAG |
| | CAATTCATCCGCTACCTCATCTCCTCCAATGTTGGCGAGGTCGTCTGCATCTTCCTCACGGCAATTC |
| | TGGGCCTGCCCGAAGCCCTGATCCCTGTGCAGCTGCTCTGGGTGAACCTGGTGACAGACGGCCTACC |
| | TGCCACGGCTCTGGGCTTCAACCCGCCAGACCTGGACATCATGGAGAAGCTGCCCCGGAGCCCCCGA |
| | GAAGCCCTCATCAGTGGCTGGCTCTTCTTCCGATACCTGGCTATCGGAGTGTACGTAGGCCTGGCCA |
| | CAGTGGCTGCCGCCACCTGGTGGTTTGTGTATGACGCCGAGGGACCTCACATCAACTTCTACCAGCT |
| | GAGGAACTTCCTGAAGTGCTCCGAAGACAACCCGCTCTTTGCCGGCATCGACTGTGAGGTGTTCGAG |
| | TCACGCTTCCCCACCACCATGGCCTTGTCCGTGCTCGTGACCATTGAAATGTGCAATGCCCTCAACA |
| | GCGTCTCGGAGAACCAGTCGCTGCTGCGGATGCCGCCCTGGATGAACCCCTGGCTGCTGGTGGCTGT |
| | GGCCATGTCCATGGCCCTGCACTTCCTCATCCTGCTCGTGCCGCCCCTGCCTCTCATTTTCCAGGTG |
| | ACCCCACTGAGCGGGCGCCAGTGGGTGGTGGTGCTCCAGATATCTCTGCCTGTCATCCTGCTGGATG |
| | AGGCCCTCAAGTACCTGTCCCGGAACCACATGCACGAAGAAATGAGCCAGAAGTGA |

| | | |
|---|---|---|
| ORF Start: at 2 | | ORF Stop: TGA at 3002 |
| SEQ ID NO: 220 | 1000 aa | MW at 109325.9 kD |

| | |
|---|---|
| NOV25c, | TMEAAHLLPAADVLRHFSVTAEGGLSPAQVTGARERYGPNELPSEEGKSLWELVLEQFEDLLVRILL |
| 248851003 | |
| Protein Sequence | LAALVSFVLAWFEEGEETTTAFVEPLVIMLILVANAIVGVWQERNAESAIEALKEYEPEMGKVIRSD |
| | RKGVQRIRARDIVPGDIVEVAVGDKVPADLRLIEIKSTTLRVDQSILTGESVSVTKHTEAIPDPRAV |
| | NQDKKNMLFSGTNITSGKAVGVAVATGLHTELGKIRSQMAAVEPERTPLQRKLDEFGRQLSHAISVI |
| | CVAVWVINIGHFADPAHGGSWLRGAVYYFKIAVALAVAAIPEGLPAVITTCLALGTRRMARKNAIVR |
| | SLPSVETLGCTSVICSDKTGTLTTNQMSVCRMFVVAEADAGSCLLHEFTISGTTYTPEGEVRQGDQP |
| | VRCGQFDGLVELATICALCNDSALDYNEAKGVYEKVGEATETALTCLVEKMNVFDTDLQALSRVERA |
| | GACNTVIKQLMRKEFTLEFSRDRKSMSVYCTPTRPHPTGQGSKMFVKGAPESVIERCSSVRVGSRTA |
| | PLTPTSREQILAKIRDWGSGSDTLRCLALATRDAPPRKEDMELDDCGKFVQYETDLTFVGCVGMLDP |
| | PRPEVAACITRCYQAGIRVVMITGDNKGTAVAICRRLGIFGDTEDVAGKAYTGREFDDLSPEQQRQA |
| | CRTARCFARVEPAHKSRIVENLQSFNEITAMTGDGVNDAPALKKAEIGIAMGSGTAVAKSAAEMVLS |
| | DDNFASIVAAVEEGRAIYSNMKQFIRYLISSNVGEVVCIFLTAILGLPEALIPVQLLWVNLVTDGLP |
| | ATALGFNPPDLDIMEKLPRSPREALISGWLFFRYLAIGVYVGLATVAAATWWFVYDAEGPHINFYQL |
| | RNFLKCSEDNPLFAGIDCEVFESRFPTTMALSVLVTIEMCNALNSVSENQSLLRMPPWMNPWLLVAV |
| | AMSMALHFLILLVPPLPLIFQVTPLSGRQWVVVLQISLPVILLDEALKYLSRNHMHEEMSQK |

| | |
|---|---|
| SEQ ID NO: 221 | 4553 bp |

| | |
|---|---|
| NOV25d, | GGCGGCATGGAGGCGGCGCATCTGCTCCCGGCCGCCGACGTGCTGCGCCACTTCTCGGTGACAGCCG |
| CG56216-02 | |
| DNA Sequence | AGGGCGGCCTGAGCCCGGCGCAGGTGACCGGCGCGCGGGAGCGCTACGGCCCCAACGAGCTCCCGAG |
| | TGAGGAAGGGAAGTCCCTGTGGGAGCTGGTGCTGGAACAGTTTGAGGACCTCCTGGTGCGCATCCTG |
| | CTGCTGGCTGCCCTTGTCTCCTTTGTCCTGGCCTGGTTCGAGGAGGGCGAGGAGACCACGACCGCCT |
| | TCGTGGAGCCCCTGGTCATCATGCTGATCCTCGTGGCCAACGCCATTGTGGGCGTGTGGCAGGAACG |
| | CAACGCCGAGAGTGCCATCGAGGCCCTGAAGGAGTATGAGCCTGAGATGGGCAAGGTGATCCGCTCG |
| | GACCGCAAGGGCGTGCAGAGGATCCGTGCCCGGGACATCGTCCCAGGGGACATTGTAGAAGTGGCAG |
| | TGGGGGACAAAGTGCCTGCTGACCTCCGCCTCATCGAGATCAAGTCCACCACGCTGCGAGTGGACCA |
| | GTCCATCCTGACGGGTGAATCTGTGTCCGTGACCAAGCACACAGAGGCCATCCCAGACCCCAGAGCT |
| | GTGAACCAGGACAAGAAGAACATGCTGTTTTCTGGCACCAATATCACATCGGGCAAAGCGGTGGGTG |
| | TGGCCGTGGCCACCGGCCTGCACACGGAGCTGGGCAAGATCCGGAGCCAGATGGCGGCAGTCGAGCC |
| | CGAGCGGACGCCGCTGCAGCGCAAGCTGGACGAGTTTGGACGGCAGCTGTCCCACGCCATCTCTGTG |
| | ATCTGCGTGGCCGTGTGGGTCATCAACATCGGCCACTTCGCCGACCCGGCCCACGGTGGCTCCTGGC |
| | TGCGTGGCGCTGTCTACTACTTCAAGATCGCCGTGGCCCTGGCGGTGGCGGCCATCCCCGAGGGCCT |
| | CCCGGCTGTCATCACTACATGCCTGGCACTGGGCACGCGGCGCATGGCACGCAAGAACGCCATCGTG |
| | CGAAGCCTGCCGTCCGTGGAGACCCTGGGCTGCACCTCAGTCATCTGCTCCGACAAGACGGGCACGC |
| | TCACCACCAATCAGATGTCTGTCTGCCGGATGTTCGTGGTAGCCGAGGCCGATGCGGGCTCCTGCCT |
| | TTTGCACGAGTTCACCATCTCGGGTACCACGTATACCCCCGAGGGCGAAGTGCGGCAGGGGGATCAG |
| | CCTGTGCGCTGCGGCCAGTTCGACGGGCTGGTGGAGCTGGCGACCATCTGCGCCCTGTGCAACGACT |
| | CGGCGCTGGACTACAACGAGGCCAAGGGTGTGTACGAGAAGGTGGGAGAGGCCACGGAGACAGCTCT |
| | GACTTGCCTGGTGGAGAAGATGAATGTGTTCGACACCGACCTGCAGGCTCTGTCCCGGGTGGAGCGA |
| | GCTGGCGCCTGTAACACGGTCATCAAGCAGCTGATGCGGAAGGAGTTCACCCTGGAGTTCTCCCGAG |
| | ACCGGAAATCCATGTCCGTGTACTGCACGCCCACCCGCCCTCACCCTACCGGCCAGGGCAGCAAGAT |
| | GTTTGTGAAGGGGGCTCCTGAGAGTGTGATCGAGCGCTGTAGCTCAGTCCGCGTGGGGAGCCGCACA |
| | GCACCCCTGACCCCCACCTCCAGGGAGCAGATCCTGGCAAAGATCCGGGATTGGGGCTCAGGCTCAG |
| | ACACGCTGCGCTGCCTGGCACTGGCCACCCGGGACGCGCCCCCAAGGAAGGAGGACATGGAGCTGGA |
| | GCTGGCGCCTGTAACACGGTCATCAAGCAGCTGATGCGGAAGGAGTTCACCCTGGAGTTCTCCCGAC |
| | ACCGGAAATCCATGTCCGTGTACTGCACGCCCACCCGCCCTCACCCTACCGGCCAGGGCAGCAAGAT |
| | GTTTGTGAAGGGGGCTCCTGAGAGTGTGATCGAGCGCTGTAGCTCAGTCCGCGTGGGGAGCCGCACA |
| | GCACCCCTGACCCCCACCTCCAGGGAGCAGATCCTGGCAAAGATCCGGGATTGGGGCTCAGGCTCAG |
| | ACACGCTGCGCTGCCTGGCACTGGCCACCCGGGACGCGCCCCCAAGGAAGGAGGACATGGAGCTGGA |
| | CGACTGCAGCAAGTTTGTGCAGTACGAGACGGACCTGACCTTCGTGGGCTGCGTAGGCATGCTGGAC |
| | CCGCCGCGACCTGAGGTGGCTGCCTGCATCACACGCTGCTACCAGGCGGGCATCCGCGTGGTCATGA |
| | TCACGGGGGATAACAAAGGCACTGCCGTGGCCATCTGCCGCAGGCTTGGCATCTTTGGGGACACGGA |
| | AGACGTGGCGGGCAAGGCCTACACGGGCCGCGAGTTTGATGACCTCAGCCCCGAGCAGCAGCGCCAG |
| | GCCTGCCGCACCGCCCGCTGCTTCGCCCGCGTGGAGCCCGCACACAAGTCCCGCATCGTGGAGAACC |
| | TGCAGTCCTTTAACGAGATCACTGCTATGACTGGCGATGGAGTGAACGACGCACCAGCCCTGAAGAA |
| | AGCAGAGATCGGCATCGCCATGGGCTCAGGCACGGCCGTGGCCAAGTCGGCGGCAGAGATGGTGCTG |
| | TCAGATGACAACTTTGCCTCCATCGTGGCTGCGGTGGAGGAGGGCCGGGCCATCTACAGCAACATGA |
| | AGCAATTCATCCGCTACCTCATCTCCTCCAATGTTGGCGAGGTCGTCTGCATCTTCCTCACGGCAAT |
| | TCTGGGCCTGCCCGAAGCCCTGATCCCTGTGCAGCTGCTCTGGGTGAACCTGGTGACAGATGGCCTA |
| | CCTGCCACGGCTCTGGGCTTCAACCCGCCAGACCTGGACATCATAGAGAAGCTGCCCCGGAGCCCCC |
| | GAGAAGCCCTCATCAGTGGCTGGCTCTTCTTCCGATACCTGGCTATCGGAGTGTACGTAGGCCTGGC |
| | CACAGTGGCTGCCGCCACCTGGTGGTTTGTGTATGACGCCGAGGGACCTCACATCAACTTCTACCAG |
| | CTGAGGAACTTCCTGAAGTGCTCCGAAGACAACCCGCTCTTTGCCGGCATCGACTGTGAGGTGTTCG |
| | AGTCACGCTTCCCCACCACCATGGCCTTGTCCGTGCTCGTGACCATTGAAATGTGCAATGCCCTCAA |
| | CAGCGTCTCGGAGAACCAGTCGCTGCTGCGGATGCCGCCCTGGATGAACCCCTGGCTGCTGGTGGCT |
| | GTGGCCATGTCCATGGCCCTGCACTTCCTCATCCTGCTCGTGCCGCCCCTGCCTCTCATTTTCCAGG |
| | TGACCCCACTGAGCGGGCGCCAGTGGGTGGTGGTGCTCCAGATATCTCTGCCTGTCATCCTGCTGGA |
| | TGAGGCCCTCAAGTACCTGTCCCGGAACCACATGCACGAAGAAATGAGCCAGAAGTGAGCGCTGGGA |
| | ACAGGGTGGAGTCTCCGGTGTGTACCTCAGACTGATGGTGCCCATGTGTTCGCCTCCGCCCCCCACC |
| | CTTGCCACCACACTCGCCCACTTGCCCACCGGGTCCCGCCGGATAAATGACAGGCCCGAGGTCAGAA |
| | TGGCCATCCCCGGGCCCCGTCCTGGGGTCTCTGTCCCCACTTCCTTCTGGCCTGGGAGGTCTGTAAT |
| | TCCTGTCTCCTGGACTCTCCTGGGAAGTTCCCTGCTCTGCAGCTCTGGCCCAGGAGCTGCAGGCTGG |
| | GAGGGGGCAGCCAAGAAGCCGGAGCTGGCAGCATACCCAGAGATCCGGGGCCCCCCCACCCCCAAAT |
| | CACGAGTGCAGCTGGAGCTTGCTCCCCCTTGTTCGGAAGCTGGACGTTCACTTGGTGACTGGTGCCT |
| | CTGCACTGACGGAGGACTCTGGGGGTCCTTCTTACCGGCTCTGACCTCTCTCTTCGTGCCTGGTCTG |
| | GGACTGGGTCAGCCCTGGGGGATCAGAAGGGGCCATCTGGGCCCAGCTGTGTACAGCGAGGGTGGGC |
| | AGCCCCCTCCACTCCACTCTGCTTCCACAAAGTCGGCTCCCGAGAGCTCGAGGCTGCTTCTGTTTAT |
| | ATGTGCAGGGCCCGGGCCGGTGAAGGGTCAGAGAGACGGACACAAGGAGCCGGCAGGAGGGCGGAGC |
| | GAGGATGTCCTTTCCCGGGAGACAAGTCGGGAAAGCCTGGCTGGACTGCCTCAGCCCCGCGCGCCTC |
| | CTGGACTCAGGGTTCCCCGTCCTGAGCTCGGGAGATGTTCAGAGTCACACTGCCGCCCGGTCTGCCA |
| | CGCAGAGGTCCAACTTGCCACCCGCGTCCCTGGTACCTGAGACCACCGACATCCTCAGGTTCCTGAC |
| | CGTGGCGCCCTTCTACCCAGCCCAGTGTGCGGCCGCCGCGCTGTCTGCACAGCTGGGGGCCTCTGAG |
| | CCTGGTGGGCTTCCTGGACTCTTGGCCTCACTCCTTGCCCCCTCCCCACGACACCCATGAGCCGAAA |
| | GGATGTCACTAAGGATGGCTGATTCCCCAAGGGCACCCGCTCTCCCTCCCTCCCTGCTGGAGGAACA |
| | CGTCATATCAGATGAGAGGAAGATGGCCTCTGATGGACAGAATTTTTCTCTTAACTCAGCTTTTGCT |
| | ACTTTGGCAAAAACTAGCGAGGGGTAGCAGAAACCTGCACCAAGGATTGTCCCTATGTCTTGGCCCC |
| | TCCTAGAGCGTGTGCAGACTGATGATTTTATATGTAAATCAAGACTCACATCCCTTTCCTAGTCCCC |
| | CACATCCAAAGCCCCTCAGCCTGCCTTGCAGACCAATGGGCTCCATGTTCTGTAGCCCCCTCCCCTA |
| | CGCCTCACCCCTCCTCCCTCTCACAGGTTCTGGGCGGCCAGTGAGAGAAACGCAGTGGGGGAGGCAG |
| | GGAGTCTGGTGCCTGCAGAGATTCTCTGCTTCTTTCCTGGGGGGAGGTGGGGAGGTCTTAGCAGGAG |
| | CGGGCCCTGTACCCACCTGCTGACCTGCTGTTTGGTAGAGAAATAAAGGTTGTGTGACTGGGGG |

| | | |
|---|---|---|
| ORF Start: ATG at 7 | | ORF Stop: TGA at 3004 |
| SEQ ID NO: 222 | 999 aa | MW at 109236.8 kD |

| | |
|---|---|
| NOV25d, | MEAAHLLPAADVLRHFSVTAEGGLSPAQVTGARERYGPNELPSEEGKSLWELVLEQFEDLLVRILLL |
| CG56216-02 | |
| Protein Sequence | AALVSFVLAWFEEGEETTTAFVEPLVIMLILVANAIVGVWQERNAESAIEALKEYEPEMGKVIRSDR |
| | KGVQRIRARDIVPGDIVEVAVGDKVPADLRLIEIKSTTLRVDQSILTGESVSVTKHTEAIPDPRAVN |
| | QDKKNMLFSGTNITSGKAVGVAVATGLHTELGKIRSQMAAVEPERTPLQRKLDEFGRQLSHAISVIC |
| | VAVWVINIGHFADPAHGGSWLRGAVYYFKIAVALAVAAIPEGLPAVITTCLALGTRRMARKNAIVRS |
| | LPSVETLGCTSVICSDKTGTLTTNQMSVCRMFVVAEADAGSCLLHEFTISGTTYTPEGEVRQGDQPV |
| | RCGQFDGLVELATICALCNDSALDYNEAKGVYEKVGEATETALTCLVEKMNVFDTDLQALSRVERAG |
| | ACNTVIKQLMRKEFTLEFSRDRKSMSVYCTPTRPHPTGQGSKMFVKGAPESVIERCSSVRVGSRTAP |
| | LTPTSREQILAKIRDWGSGSDTLRCLALATRDAPPRKEDMELDDCSKFVQYETDLTFVGCVGMLDPP |
| | RPEVAACITRCYQAGIRVVMITGDNKGTAVAICRRLGIFGDTEDVAGKAYTGREFDDLSPEQQRQAC |
| | RTARCFARVEPAHKSRIVENLQSFNEITAMTGDGVNDAPALKKAEIGIAMGSGTAVAKSAAEMVLSD |
| | DNFASIVAAVEEGRAIYSNMKQFIRYLISSNVGEVVCIFLTAILGLPEALIPVQLLWVNLVTDGLPA |
| | TALGFNPPDLDIIEKLPRSPREALISGWLFFRYLAIGVYVGLATVAAATWWFVYDAEGPHINFYQLR |
| | NFLKCSEDNPLFAGIDCEVFESRFPTTMALSVIVTIEMCNALNSVSENQSLLRMPPWMNPWLLVAVA |
| | MSMALHFLILLVPPLPLIFQVTPLSGRQWVVVLQISLPVILLDEALKYLSRNHMHEEMSQK |

Sequence comparison of the above protein sequences yields the following sequence relationships shown in Table 25B.

TABLE 25B
Comparison of NOV25a against NOV25b through NOV25d.

| | NOV25a Residues/ | Identities/Similarities |
|---|---|---|
| Protein Sequence | Match Residues | for the Matched Region |
| NOV25b | 1 . . . 999 | 999/999 (100%) |
| | 2 . . . 1000 | 999/999 (100%) |
| NOV25c | 1 . . . 999 | 999/999 (100%) |
| | 2 . . . 1000 | 999/999 (100%) |
| NOV25d | 1 . . . 999 | 997/999 (99%) |
| | 1 . . . 999 | 998/999 (99%) |

Further analysis of the NOV25a protein yielded the following properties shown in Table 25C.

TABLE 25C
Protein Sequence Properties NOV25a

| SignalP | | |
|---|---|---|
| analysis: | Cleavage site between residues 23 and 24 | |
| PSORT II | PSG: | a new signal peptide prediction method |
| analysis: | | N-region: length 11; pos. chg 0; neg. chg 2 |
| | | H-region: length 2; peak value 0.00 |
| | | PSG score: −4.40 |
| | GvH: | von Heijne's method for signal seq. recognition |
| | | GvH score (threshold: −2.1): −8.71 |
| | | possible cleavage site: between 31 and 32 |
| | >>> Seems to have no N-terminal signal peptide | |
| | ALOM: | Klein et al's method for TM region allocation |

| Init position for calculation: 1 | | | |
|---|---|---|---|
| Tentative number of TMS(s) for the threshold 0.5: 8 | | | |
| INTEGRAL | Likelihood = | −10.46 | |
| Transmembrane 60-76 | | | |
| INTEGRAL | Likelihood = | −7.70 | |
| Transmembrane 88-104 | | | |
| INTEGRAL | Likelihood = | −4.57 | |
| Transmembrane 260-276 | | | |
| INTEGRAL | Likelihood = | −3.19 | |
| Transmembrane 298-314 | | | |
| INTEGRAL | Likelihood = | −6.85 | |
| Transmembrane 772-788 | | | |
| INTEGRAL | Likelihood = | −1.28 | |
| Transmembrane 838-854 | | | |
| INTEGRAL | Likelihood = | −9.13 | |
| Transmembrane 934-950 | | | |
| INTEGRAL | Likelihood = | −3.61 | |
| Transmembrane 968-984 | | | |
| PERIPHERAL | Likelihood = | 1.38 | (at 897) |

| | ALOM score: −10.46 (number of TMSs: 8) |
|---|---|
| MTOP: | Prediction of membrane topology (Hartmann et al.) |
| | Center position for calculation: 67 |
| | Charge difference: −2.0 C(−5.0) − N(−3.0) |
| | N >= C: N-terminal side will be inside |

>>> membrane topology: type 3a
MITDISC: discrimination of mitochondrial targeting seq

| | | | |
|---|---|---|---|
| R content: | 0 | Hyd Moment | 1.14 |
| Hyd Moment(95): | 5.42 | (75): | |
| D/E content: | 2 | G content: | 0 |
| Score: | −7.82 | S/T content: | 0 |

| Gavel: | prediction of cleavage sites for mitochondrial preseq |
|---|---|
| | R-10 motif at 24 LRH FS |
| NUCDISC: discrimination of nuclear localization signals | |
| | pat4: none |
| | pat7: none |
| | bipartite: none |
| | content of basic residues: 9.7% |
| | NLS Score: −0.47 |
| KDEL: | ER retention motif in the C-terminus: none |
| ER Membrane Retention Signals: none | |
| SKL: | peroxisomal targeting signal in the C-terminus: none |
| PTS2: | 2nd peroxisomal targeting signal: found |
| | KLDEFGRQL at 252 |
| VAC: | possible vacuolar targeting motif: none |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

----------------------------------
Final Results (k = 9/23):

44.4%: endoplasmic reticulum
22.2%: vesicles of secretory system
11.1%: vacuolar
11.1%: Golgi
11.1%: mitochondrial

>> prediction for CG56216-01 is end (k = 9)

A search of the NOV25a protein against the Geneseq database, a proprietary database that contains sequences published in patents and patent publication, yielded several homologous proteins shown in Table 25D.

TABLE 25D
Geneseq Results for NOV25a

| | | | Identities/ | |
|---|---|---|---|---|
| | | NOV25a | Similarities | |
| | Protein/Organism/ | Residues/ | for the | |
| Geneseq | Length | Match | Matched | Expect |
| Identifier | [Patent #, Date] | Residues | Region | Value |
| AAB90764 | Human shear stress- | 1 . . . 992 | 767/992 | 0.0 |
| | response protein | | (77%) | |
| | SEQ ID NO:28 - | 1 . . . 991 | 871/992 | |
| | Homo sapiens, | | (87%) | |
| | 997 aa. | | | |
| | [WO200125427-A1, | | | |
| | 12-APR-2001] | | | |
| AAM78337 | Human protein SEQ | 1 . . . 999 | 758/999 | 0.0 |
| | ID NO 999 - Homo | | (75%) | |
| | sapiens, 1001 aa. | 1 . . . 999 | 878/999 | |
| | [WO200157190-A2, | | (87%) | |
| | 09-AUG-2001] | | | |
| ABB09807 | Amino acid | 1 . . . 992 | 758/992 | 0.0 |
| | sequence of human | | (76%) | |
| | SERCA 1 - Homo | 1 . . . 992 | 875/992 | |
| | sapiens, 994 aa. | | (87%) | |
| | [WO200222777-A2, | | | |
| | 21-MAR-2002] | | | |
| AAM79321 | Human protein SEQ | 1 . . . 992 | 757/992 | 0.0 |
| | ID NO 2967 - | | (76%) | |
| | Homo sapiens, | 58 . . . 1049 | 874/992 | |
| | 1072 aa. | | (87%) | |
| | [WO200157190-A2, | | | |
| | 09-AUG-2001] | | | |
| ABB66626 | Drosophila | 1 . . . 989 | 673/989 | 0.0 |
| | melanogaster | | (68%) | |
| | polypeptide | 1 . . . 989 | 794/989 | |
| | SEQ ID NO 26670 - | | (80%) | |
| | Drosophila | | | |
| | melanogaster, | | | |
| | 1020 aa. | | | |
| | [WO200171042-A2, | | | |
| | 27-SEP-2001] | | | |

In a BLAST search of public sequence datbases, the NOV25a protein was found to have homology to the proteins shown in the BLASTP data in Table 25E.

TABLE 25E
Public BLASTP Results for NOV25a

| | | NOV25a | Identities | |
|---|---|---|---|---|
| Protein | | Residues/ | Similarities for | |
| Accession | Protein/Organism/ | Match | the Matched | Expect |
| Number | Length | Residues | Portion | Value |
| AAH35729 | Hypothetical | 1 . . . 999 | 998/999 (99%) | 0.0 |
| | protein - Homo | 1 . . . 999 | 998/999 (99%) | |
| | sapiens (Human), | | | |
| | 999 aa. | | | |
| S72267 | Ca2+-transporting | 1 . . . 999 | 997/999 (99%) | 0.0 |
| | ATPase | 1 . . . 999 | 998/999 (99%) | |
| | (EC 3.6.1.38) | | | |
| | isoform SERCA3, | | | |
| | sarcoplasmic/ | | | |
| | endoplasmic | | | |
| | reticulum - human, | | | |
| | 999 aa. | | | |
| Q93084 | Sarcoplasmic/ | 1 . . . 993 | 992/993 (99%) | 0.0 |
| | endoplasmic | 1 . . . 993 | 992/993 (99%) | |
| | reticulum calcium | | | |
| | ATPase 3 | | | |
| | (EC 3.6.3.8) | | | |
| | (Calcium pump 3) | | | |
| | (SERCA3) (SR | | | |
| | Ca(2+)-ATPase 3) - | | | |
| | Homo sapiens | | | |
| | (Human), 1043 aa. | | | |
| Q8R0X5 | Similar to ATPase, | 1 . . . 999 | 947/999 (94%) | 0.0 |
| | Ca++ transporting, | 1 . . . 999 | 967/999 (96%) | |
| | ubiquitous - Mus | | | |
| | musculus (Mouse), | | | |
| | 999 aa. | | | |
| Q64518 | Sarcoplasmic/ | 1 . . . 992 | 946/992 (95%) | 0.0 |
| | endoplasmic | 1 . . . 992 | 965/992 (96%) | |
| | reticulum calcium | | | |
| | ATPase 3 | | | |
| | (EC 3.6.3.8) | | | |
| | (Calcium pump 3) | | | |
| | (SERCA3) (SR | | | |
| | Ca(2+)-ATPase 3) - | | | |
| | Mus musculus | | | |
| | (Mouse), 1038 aa. | | | |

PFam analysis predicts that the NOV25a protein contains the domains shown in the Table 25F.

TABLE 25F
Domain Analysis of NOV25a

| | | Identities/ | |
|---|---|---|---|
| | | Similarities | |
| | NOV25a Match | for the Matched | Expect |
| Pfam Domain | Region | Region | Value |
| Cation_ATPase_N | 2 . . . 77 | 28/87 (32%) | 2e−14 |
| | | 56/87 (64%) | |
| E1-E2_ATPase | 93 . . . 341 | 120/250 (48%) | 1.2e−124 |
| | | 231/250 (92%) | |
| Hydrolase | 345 . . . 724 | 44/390 (11%) | 1.3e−17 |
| | | 245/390 (63%) | |
| Cation_ATPase_C | 819 . . . 996 | 88/194 (45%) | 3.7e−77 |
| | | 154/194 (79%) | |

### Example 26

The NOV26 clone was analyzed, and the nucleotide and encoded polypeptide sequences are shown in Table 26A.

TABLE 26A
NOV26 Sequence Analysis

| | |
|---|---|
| SEQ ID NO: 223 | 911 bp |

| | |
|---|---|
| NOV26a, | AGAGTTCATTCTGCAAGGACTTTCAGGGTACCCAAGAGCTGAAAAATTCCTTTTCGTGATGTGCTTA |
| CG56230-01 | |
| DNA Sequence | GTGAGGTACCTGGTGATTCTCCTAGGGAATGGCACCTTGATCATTCTGACACTCCTGGATGCTCGTC |
| | TCCACACACCCATGTACTTCTTCCTTGGGAATCTCTCCTTCCTAGACATTTGGTACACATCCTCCTC |
| | CATCCCCTCAATGCTGATACACTTCCCATCAGAGAAGAAAACCATTTCCTTCACTAGATGTGTGATT |
| | CAAATGTCTGTCTCTTACACTATGGGATCCACCAAGTGTGTGCTTCTAGCAGTGATGGCATATGACC |
| | GTTATGTAGCCATCTGCAACCCTCTGAGATATCCCATCATCATGGGCAAGGCACTTTGTATTCAGAT |
| | GGTGGCTGTCTCTTGGGGACTAGGCTTTCTCAACTCATTGACAGAAACTGTTCTTGCAATACGGTTA |
| | CCCTTCTGTGGAAAAAATGTCATCAATCATTTTGTTTGTGAAATATTGGCCTTTGTCAAGCTGGCTT |
| | GCACAGATACTTCCTTGAATGAGATTATTATAATGTTGGGCAATGTAATATTTTTGTTTTCTCCATT |
| | ACTGCTGATTTGTATCTCCTACATCTTTATCCTTTCTACTGTACTAAGAATCAATTCAGCTGAAGGA |
| | AGGAAAAAGGCCTTTTCCACCTGCTCAGCCCACATGACAGTGGTGATTGTGTTTTATGGGACAATCC |
| | TCTTCATGTACATGAAGGCAAAGTCCAAAGACTCTGCTTTTGACAAACTGATTGCCCTGTTCTATGG |
| | CATAGTCACCCCCATGCCCAATCCTATCATCTACAGCCTGAGGAATACAGAGGTGCATGGAGCTATG |
| | AGGAAATTAATGAGTAGACCCTGGTTCTGGAGGAAATGAT |

| | | |
|---|---|---|
| ORF Start: at 2 | | ORF Stop: TGA at 908 |
| SEQ ID NO: 224 | 302 aa | MW at 34205.8 kD |

| | |
|---|---|
| NOV26a, | EFILQGLSGYPRAEKFLFVMCLVMYLVILLGNGTLIILTLLDARLHTPMYFFLGNLSFLDIWYTSSS |
| CG56230-01 | |
| Protein Sequence | IPSMLIHFPSEKKTISFTRCVIQMSVSYTMGSTKCVLLAVMAYDRYVAICNPLRYPIIMGKALCIQM |
| | VAVSWGLGFLNSLTETVLAIRLPFCGKNVINHFVCEILAFVKLACTDTSLNEIIIMLGNVIFLFSPL |
| | LLICISYIFILSTVLRINSAEGRKKAFSTCSAHMTVVIVFYGTILFMYMKAKSKDSAFDKLIALFYG |
| | IVTPMPNPIIYSLRNTEVHGAMRKLMSRPWFWRK |

Further analysis of the NOV26a protein yielded the following properties shown in Table 26B.

TABLE 26B
Protein Sequence Properties NOV26a

| SignalP | | |
|---|---|---|
| analysis: | Cleavage site between residues 34 and 35 | |
| PSORT II | PSG: | a new signal peptide prediction method |
| analysis: | | N-region: length 1; pos. chg 0; neg. chg 1 |
| | | H-region: length 10; peak value 0.00 |
| | | PSG score: −4.40 |
| | GvH: | von Heijne's method for signal seq. recognition |
| | | GvH score (threshold: −2.1): −5.65 |
| | | possible cleavage site: between 47 and 48 |
| | >>> Seems to have no N-terminal signal peptide | |
| | ALOM: | Klein et al's method for TM region allocation |

| Init position for calculation: 1 | | | |
|---|---|---|---|
| Tentative number of TMS(s) for the threshold 0.5: 4 | | | |
| INTEGRAL | Likelihood = | −8.44 | |
| Transmembrane 16-32 | | | |
| INTEGRAL | Likelihood = | −1.28 | |
| Transmembrane 163-179 | | | |
| INTEGRAL | Likelihood = | −9.13 | |
| Transmembrane 195-211 | | | |
| INTEGRAL | Likelihood = | −1.70 | |
| Transmembrane 231-247 | | | |
| PERIPHERAL | Likelihood = | 0.63 | (at 124) |

| | ALOM score: −9.13 (number of TMSs: 4) |
|---|---|
| MTOP: | Prediction of membrane topology (Hartmann et al.) |
| | Center position for calculation: 23 |
| | Charge difference: −0.5 C(0.5) − N(1.0) |
| | N >= C: N-terminal side will be inside |

>>> membrane topology: type 3a
MITDISC: discrimination of mitochondrial targeting seq

| | | | |
|---|---|---|---|
| R content: | 1 | Hyd Moment | 8.79 |
| Hyd Moment(95): | 9.06 | (75): | |
| D/E content: | 3 | G content: | 4 |
| Score: | −8.47 | S/T content: | 3 |

| Gavel: | prediction of cleavage sites for mitochondrial preseq |
|---|---|
| | R-2 motif at 165 IRL\|PF |
| NUCDISC: discrimination of nuclear localization signals | |
| | pat4: none |
| | pat7: none |
| | bipartite: none |
| | content of basic residues: 8.9% |
| | NLS Score: −0.47 |
| KDEL: | ER retention motif in the C-terminus: none |
| ER Membrane Retention Signals: none | |
| SKL: | peroxisomal targeting signal in the C-terminus: none |
| PTS2: | 2nd peroxisomal targeting signal: none |
| VAC: | possible vacuolar targeting motif: none |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

----------------------------------
Final Results (k = 9/23):

```
52.2%: endoplasmic reticulum
34.8%: mitochondrial
  8.7%: nuclear
  4.3%: vesicles of secretory system
```

>> prediction for CG56230-01 is end (k = 23)

A search of the NOV26a protein against the Geneseq database, a proprietary database that contains sequences published in patents and patent publication, yielded several homologous proteins shown in Table 26C.

TABLE 26C
Geneseq Results for NOV26a

| | | | Identities/ | |
|---|---|---|---|---|
| | | NOV26a | Similarities | |
| | Protein/Organism/ | Residues/ | for the | |
| Geneseq | Length | Match | Matched | Expect |
| Identifier | [Patent #, Date] | Residues | Region | Value |
| ABB06655 | G protein-coupled | 1 . . . 302 | 302/302 | e−174 |
| | receptor GPCR32b | | (100%) | |
| | protein SEQ ID | 1 . . . 302 | 302/302 | |
| | NO:120 - Homo | | (100%) | |
| | sapiens, 302 aa. | | | |
| | [WO200212343-A2, | | | |
| | 14-FEB-2002] | | | |
| ABB06656 | G protein-coupled | 1 . . . 302 | 299/302 | e−171 |
| | receptor GPCR33 | | (99%) | |
| | protein SEQ ID | 10 . . . 311 | 300/302 | |
| | NO:122 - Homo | | (99%) | |
| | sapiens, 311 aa. | | | |
| | [WO200212343-A2, | | | |
| | 14-FEB-2002] | | | |
| AAG71954 | Human olfactory | 1 . . . 296 | 293/296 | e−166 |
| | receptor | | (98%) | |
| | polypeptide, SEQ | 10 . . . 305 | 294/296 | |
| | ID NO:1635 - | | (98%) | |
| | Homo sapiens, | | | |
| | 333 aa. | | | |
| | [WO200127158-A2, | | | |
| | 19-APR-2001] | | | |
| AAG72651 | Murine OR-like | 1 . . . 301 | 196/311 | e−108 |
| | polypeptide query | | (63%) | |
| | sequence, SEQ | 28 . . . 338 | 240/311 | |
| | ID NO:2333 - | | (77%) | |
| | Mus musculus, | | | |
| | 356 aa. | | | |
| | [WO200127158-A2, | | | |
| | 19-APR-2001] | | | |
| AAG72652 | Murine OR-like | 1 . . . 296 | 193/303 | e−108 |
| | polypeptide query | | (63%) | |
| | sequence, SEQ | 24 . . . 326 | 238/303 | |
| | ID NO:2334 - | | (77%) | |
| | Mus musculus, | | | |
| | 331 aa. | | | |
| | [WO200127158-A2, | | | |
| | 19-APR-2001] | | | |

In a BLAST search of public sequence datbases, the NOV26a protein was found to have homology to the proteins shown in the BLASTP data in Table 26D.

TABLE 26D
Public BLASTP Results for NOV26a

| | | | Identities/ | |
|---|---|---|---|---|
| | | NOV26a | Similarities | |
| Protein | | Residues/ | for the | |
| Accession | Protein/Organism/ | Match | Matched | Expect |
| Number | Length | Residues | Portion | Value |
| CAD42438 | Sequence 119 from | 1 . . . 302 | 302/302 | e−174 |
| | Patent | | (100%) | |
| | WO0212343 - | 1 . . . 302 | 302/302 | |
| | Homo sapiens | | (100%) | |
| | (Human), 302 aa | | | |
| | (fragment). | | | |
| CAD42439 | Sequence 121 from | 1 . . . 302 | 299/302 | e−171 |
| | Patent | | (99%) | |
| | WO0212343 - | 10 . . . 311 | 300/302 | |
| | Homo sapiens | | (99%) | |
| | (Human), 311 aa. | | | |
| Q8VFN0 | Olfactory receptor | 1 . . . 302 | 198/303 | e−112 |
| | MOR262-9 - Mus | | (65%) | |
| | musculus (Mouse), | 9 . . . 311 | 243/303 | |
| | 312 aa. | | (79%) | |
| Q9QZ22 | Olfactory receptor | 2 . . . 296 | 193/302 | e−109 |
| | GA_x5J8B7W5BNN- | | (63%) | |
| | 979337- | 13 . . . 314 | 240/302 | |
| | 980296 - | | (78%) | |
| | Mus musculus | | | |
| | (Mouse), 319 aa. | | | |
| Q9QZ20 | Olfactory receptor - | 1 . . . 295 | 194/302 | e−108 |
| | Mus musculus | | (64%) | |
| | (Mouse), 318 aa. | 11 . . . 312 | 237/302 | |
| | | | (78%) | |

PFam analysis predicts that the NOV26a protein contains the domains shown in the Table 26E.

TABLE 26E
Domain Analysis of NOV26a

| | NOV26a Match | Identities/Similarities | Expect |
|---|---|---|---|
| Pfam Domain | Region | for the Matched Region | Value |
| 7tm_1 | 31 . . . 279 | 57/268 (21%) | 2.9e−44 |
| | | 175/268 (65%) | |

### Example 27

The NOV27 clone was analyzed, and the nucleotide and encoded polypeptide sequences are shown in Table 27A.

TABLE 27A
NOV27 Sequence Analysis

| | |
|---|---|
| SEQ ID NO: 225 | 1279 bp |

| | |
|---|---|
| NOV27a, | CCACCATGGGCCACCATCACCACCATCACAGGTTGATCCTGTTTTTTGGTGCCCTTTTTGGGCATAT |
| CG56246-04 | |
| DNA Sequence | CTACTGTCTAGAAACATTTGTGGGAGACCAAGTTCTTGAGATTGTACCAAGCAATGAAGAACAAATT |
| | AAAAATCTGCTACAATTGGAGGCTCAAGAACATCTCCAGCTTGATTTTTGGAAATCACCCACCACCC |
| | CAGGGGAGACAGCCCACGTCCGAGTTCCCTTCGTCAACGTCCAGGCAGTCAAAGTGTTCTTGGAGTC |
| | CCAGGGAATTGCCTATTCCATCATGATTGAAGACGTGCAGGTCCTGTTGGACAAAGAGAATGAAGAA |
| | ATGCTTTTTAATAGGAGAAGAGAACGGAGTGGTAACTTCAATTTTGGGGCCTACCATACCCTGGAAG |
| | AGATTTCCCAAGAAATGGATAACCTCGTGGCTGAGCACCCTGGTCTAGTGAGCAAAGTGAATATTGG |
| | CTCTTCTTTTGAGAACCGGCCTATGAACGTGCTCAAGTTCAGCACCGGAGGAGACAAGCCAGCTATC |
| | TGGCTGGATGCTGGGATCCATGCTCGAGAGTGGGTTACACAAGCTACGGCACTTTGGACAGCAAATA |
| | AGATTGTTTCTGATTATGGAAAGGACCCATCCATCACTTCCATTCTGGACGCCCTGGATATCTTCCT |
| | CCTGCCAGTCACAAACCCTGATGGATACGTGTTCTCTCAAACCAAAAATCGTATGTGGCGGAAGACC |
| | CGGTCCAAGGTATCTGGAAGCCTCTGTGTTGGTGTGGATCCTAACCGGAACTGGGATGCAGGTTTTG |
| | GAGGACCTGGAGCCAGCAGCAACCCTTGCTCTGATTCATACCACGGACCCAGTGCCAACTCTGAAGT |
| | TGAAGTGAAATCCATAGTGGACTTCATCAAGAGTCATGGAAAAGTCAAGGCCTTCATTACCCTCCAC |
| | AGCTATTCCCAGCTGCTGATGTTCCCCTATGGGTACAAATGTACCAAGTTAGATGACTTTGATGAGC |
| | TGAGTGAAGTGGCCCAAAAGGCTGCCCAATCTCTGAGAAGCCTGCATGGCACCAAGTACAAAGTGGG |
| | ACCAATCTGCTCTGTCATCTACCAAGCCAGTGGAGGAAGCATTGACTGGTCCTATGATTATGGCATC |
| | AAGTACTCATTTGCCTTTGAACTGAGAGACACAGGGCGCTACGGCTTCCTCTTGCCAGCCCGTCAGA |
| | TCCTGCCCACAGCCGAGGAGACCTGGCTTGGCTTGAAGGCAATCATGGAGCATGTGCGAGACCACCC |
| | CTATTA |

| | | |
|---|---|---|
| ORF Start: at 3 | | ORF Stop: at 1278 |
| SEQ ID NO: 226 | 425 aa | MW at 47808.7 kD |

| | |
|---|---|
| NOV27a, | TMGHHHHHHRLILFFGALFGHIYCLETFVGDQVLEIVPSNEEQIKNLLQLEAQEHLQLDFWKSPTTP |
| CG56246-04 | |
| Protein Sequence | GETAGVRVPFVNVQAVKVFLESQGIAYSIMIEDVQVLLDKENEEMLFNRRREFSGVFNFGAYHTLEE |
| | ISQEMDNLVAEHPGLVSKVNIGSSFENRPMNVLKFSTGGDKPAIWLDAGIHAREWVTQATALWTANK |
| | IVSDYGKDPSITSILDALDIFLLPVTNPDGYVFSQTKNRMWRKTRSKVSGSLCVGVDPNRNWDAGFG |
| | GPGASSNPCSDSYHGPSANSEVEVKSIVDFIKSHGKVKAFITLHSYSQLLMFPYGYKCTKLDDFDEL |
| | SEVAQKAAQSLRSLHGTKYKVGPICSVIYQASGGSIDWSYDYGIKYSFAFELRDTGRYGFLLPARQI |
| | LPTAEETWLGLKAIMEHVRDHPY |

| | |
|---|---|
| SEQ ID NO: 227 | 1309 bp |

| | |
|---|---|
| NOV27b, | GGCCATGAGGTTGATCCTGTTTTTTGGTGCCCTTTTTGGGCATATCTACTGTCTAGAAACATTTGTG |
| CG56246-02 | |
| DNA Sequence | GGAGACCAAGTTCTTGAGATTGTACCAAGCAATGAAGAACAAATTAAAAATCTGCTACAATTGGAGG |
| | CTCAAGAACATCTCCAGCTTGATTTTTGGAAATCACCCACCACCCCAGGGGAGACAGCCCACGTCCG |
| | AGTTCCCTTCGTCAACGTCCAGGCAGTCAAAGTGTTCTTGGAGTCCCAGGGAATTGCCTATTCCATC |
| | ATGATTGAAGACGTGCAGGTCCTGTTGGACAAAGAGAATGAAGAAATGCTTTTTAATAGGAGAAGAG |
| | AACGGAGTGGTAACTTCAATTTTGGGGCCTACCATACCCTGGAAGAGATTTCCCAAGAAATGGATAA |
| | CCTCGTGGCTGAGCACCCTGGTCTAGTGAGCAAAGTGAATATTGGCTCTTCTTTTGAGAACCGGCCT |
| | ATGAACGTGCTCAAGTTCAGCACCGGAGGAGACAAGCCAGCTATCTGGCTGGATGCTGGGATCCATG |
| | CTCGAGAGTGGGTTACACAAGCTACGGCACTTTGGACAGCAAATAAGATTGTTTCTGATTATGGAAA |
| | GGACCCATCCATCACTTCCATTCTGGACGCCCTGGATATCTTCCTCCTGCCAGTCACAAACCCTGAT |
| | GGATACGTGTTCTCTCAAACCAAAAATCGTATGTGGCGGAAGACCCGGTCCAAGGTATCTGGAAGCC |
| | TCTGTGTTGGTGTGGATCCTAACCGGAACTGGGATGCAGGTTTTGGAGGACCTGGAGCCAGCAGCAA |
| | CCCTTGCTCTGATTCATACCACGGACCCAGTGCCAACTCTGAAGTTGAAGTGAAATCCATAGTGGAC |
| | TTCATCAAGAGTCATGGAAAAGTCAAGGCCTTCATTATCCTCCACAGCTATTCCCAGCTGCTGATGT |
| | TCCCCTATGGGTACAAATGTACCAAGTTAGATGACTTTGATGAGCTGAGTGAAGTGGCCCAAAAGGC |
| | TGCCCAATCTCTGAGAAGCCTGCATGGCACCAAGTACAAAGTGGGACCAATCTGCTCTGTCATCTAC |
| | CAAGCCAGTGGAGGAAGCATTGACTGGTCCTATGATTATGGCATCAAGTACTCATTTGCCTTTGAAC |
| | TGAGAGACACAGGGCGCTACGGCTTCCTCTTGCCAGCCCGTCAGATCCTGCCCACAGCCGAGGAGAC |
| | CTGGCTTGGCTTGAAGGCAATCATGGAGCATGTGCGAGACCACCCCTATTAGGGCCCTGGGGAAGAA |
| | ACAAGAGCCATTAAAATCTCTTTGGTTTGAAGCAAA |

| | | |
|---|---|---|
| ORF Start: ATG at 5 | | ORF Stop: TAG at 1256 |
| SEQ ID NO: 228 | 417 aa | MW at 46839.7 kD |

| | |
|---|---|
| NOV27b, | MRLILFFGALFGHIYCLETFVGDQVLEIVPSNEEQIKNLLQLEAQEHLQLDFWKSPTTPGETAHVRV |
| CG56246-02 | |
| Protein Sequence | PFVNVQAVKVFLESQGIAYSIMIEDVQVLLDKENEEMLFNRRRERSGNFNFGAYHTLEEISQEMDNL |
| | VAEHPGLVSKVNIGSSFENRPMNVLKFSTGGDKPAIWLDAGIHAREWVTQATALWTANKIVSDYGKD |
| | PSITSILDALDIFLLPVTNPDGYVFSQTKNRMWRKTRSKVSGSLCVGVDPNRNWDAGFGGPGASSNP |
| | CSDSYHGPSANSEVEVKSIVDFIKSHGKVKAFIILHSYSQLLMFPYGYKCTKLDDFDELSEVAQKAA |
| | QSLRSLHGTKYKVGPICSVIYQASGGSIDWSYDYGIKYSFAFELRDTGRYGFLLPARQILPTAEETW |
| | LGLKAIMEHVRDHPY |

| | |
|---|---|
| SEQ ID NO: 229 | 1258 bp |

| | |
|---|---|
| NOV27c, | CCACCATGAGGTTGATCCTGTTTTTTGGTGCCCTTTTTGGGCATATCTACTGTCTAGAAACATTTGT |
| 171092849 DNA | |
| Sequence | GGGAGACCAAGTTCTTGAGATTGTACCAAGCAATGAAGAACAAATTAAAAATCTGCTACAATTGGAG |
| | GCTCAAGAACATCTCCAGCTTGATTTTTGGAAATCACCCACCACCCCAGGGGAGACAGCCCACGTCC |
| | GAGTTCCCTTCGTCAACGTCCAGGCAGTCAAAGTGTTCTTGGAGTCCCAGGGAATTGCCTATTCCAT |
| | CATGATTGAAGACGTGCAGGTCCTGTTGGACAAAGAGAATGAAGAAATGCTTTTTAATAGGAGAAGA |
| | GAACGGAGTGGTAACTTCAATTTTGGGGCCTACCATACCCTGGAAGAGATTTCCCAAGAAATGGATA |
| | ACCTCGTGGCTGAGCACCCTGGTCTAGTGAGCAAAGTGAATATTGGCTCTTCTTTTGAGAACCGGCC |
| | TATGAACGTGCTCAAGTTCAGCACCGGAGGAGACAAGCCAGCTATCTGGCTGGATGCTGGGATCCAT |
| | GCTCGAGAGTGGGTTACACAAGCTACGGCACTTTGGACAGCAAATAAGATTGTTTCTGATTATGGAA |
| | AGGACCCATCCATCACTTCCATTCTGGACGCCCTGGATATCTTCCTCCTGCCAGTCACAAACCCTGA |
| | TGGATACGTGTTCTCTCAAACCAAAAATCGTATGTGGCGGAAGACCCGGTCCAAGGTATCTGGAAGC |
| | CTCTGTGTTGGTGTGGATCCTAACCGGAACTGGGATGCAGGTTTTGGAGGACCTGGAGCCAGCAGCA |
| | ACCCTTGCTCTGATTCATACCACGGACCCAGTGCCAACTCTGAAGTTGAAGTGAAATCCATAGTGGA |
| | CTTCATCAAGAGTCATGGAAAAGTCAAGGCCTTCATTACCCTCCACAGCTATTCCCAGCTGCTGATG |
| | TTCCCCTATGGGTACAAATGTACCAAGTTAGATGACTTTGATGAGCTGAGTGAAGTGGCCCAAAAGG |
| | CTGCCCAATCTCTGAGAAGCCTGCATGGCACCAAGTACAAAGTGGGACCAATCTGCTCTGTCATCTA |
| | CCAAGCCAGTGGAGGAAGCATTGACTGGTCCTATGATTATGGCATCAAGTACTCATTTGCCTTTGAA |
| | CTGAGAGACACAGGGCGCTACGGCTTCCTCTTGCCAGCCCGTCAGATCCTGCCCACAGCCGAGGAGA |
| | CCTGGCTTGGCTTGAAGGCAATCATGGAGCATGTGCGAGACCACCCCTATTA |

| | | |
|---|---|---|
| ORF Start: at 3 | | ORF Stop: end of sequence |
| SEQ ID NO: 230 | 419 aa | MW at 46928.8 kD |

| | |
|---|---|
| NOV27c, | TMRLILFFGALFGHIYCLETFVGDQVLEIVPSNEEQIKNLLQLEAQEHLQLDFWKSPTTPGETAHVR |
| 171092849 | |
| Protein Sequence | VPFVNVQAVKVFLESQGIAYSIMIEDVQVLLDKENEEMLFNRRRERSGNFNFGAYHTLEEISQEMDN |
| | LVAEHPGLVSKVNIGSSFENRPMNVLKFSTGGDKPAIWLDAGIHAREWVTQATALWTANKIVSDYGK |
| | DPSITSILDALDIFLLPVTNPDGYVFSQTKNRMWRKTRSKVSGSLCVGVDPNRNWDAGFGGPGASSN |
| | PCSDSYHGPSANSEVEVKSIVDFIKSHGKVKAFITLHSYSQLLMFPYGYKCTKLDDFDELSEVAQKA |
| | AQSLRSLHGTKYKVGPICSVIYQASGGSIDWSYDYGIKYSFAFELRDTGRYGFLLPARQILPTAEET |
| | WLGLKAIMEHVRDHPYX |

| | |
|---|---|
| SEQ ID NO: 231 | 1280 bp |

| | |
|---|---|
| NOV27d, | CCACCATGGGCCACCATCACCACCATCACAGGTTGATCCTGTTTTTTGGTGCCCTTTTTGGGCATAT |
| 183852323 DNA | |
| Sequence | CTACTGTCTAGAAACATTTGTGGGAGACCAAGTTCTTGAGATTGTACCAAGCAATGAAGAACAAATT |
| | AAAAATCTGCTACAATTGGAGGCTCAAGAACATCTCCAGCTTGATTTTTGGAAATCACCCACCACCC |
| | CAGGGGAGACAGCCCACGTCCGAGTTCCCTTCGTCAACGTCCAGGCAGTCAAAGTGTTCTTGGAGTC |
| | CCAGGGAATTGCCTATTCCATCATGATTGAAGACGTGCAGGTCCTGTTGGACAAAGAGAATGAAGAA |
| | ATGCTTTTTAATAGGAGAAGAGAACGGAGTGGTAACTTCAATTTTGGGGCCTACCATACCCTGGAAG |
| | AGATTTCCCAAGAAATGGATAACCTCGTGGCTGAGCACCCTGGTCTAGTGAGCAAAGTGAATATTGG |
| | CTCTTCTTTTGAGAACCGGCCTATGAACGTGCTCAAGTTCAGCACCGGAGGAGACAAGCCAGCTATC |
| | TGGCTGGATGCTGGGATCCATGCTCGAGAGTGGGTTACACAAGCTACGGCACTTTGGACAGCAAATA |
| | AGATTGTTTCTGATTATGGAAAGGACCCATCCATCACTTCCATTCTGGACGCCCTGGATATCTTCCT |
| | CCTGCCAGTCACAAACCCTGATGGATACGTGTTCTCTCAAACCAAAAATCGTATGTGGCGGAAGACC |
| | CGGTCCAAGGTATCTGGAAGCCTCTGTGTTGGTGTGGATCCTAACCGGAACTGGGATGCAGGTTTTG |
| | GAGGACCTGGAGCCAGCAGCAACCCTTGCTCTGATTCATACCACGGACCCAGTGCCAACTCTGAAGT |
| | TGAAGTGAAATCCATAGTGGACTTCATCAAGAGTCATGGAAAAGTCAAGGCCTTCATTACCCTCCAC |
| | AGCTATTCCCAGCTGCTGATGTTCCCCTATGGGTACAAATGTACCAAGTTAGATGACTTTGATGAGC |
| | TGAGTGAAGTGGCCCAAAAGGCTGCCCAATCTCTGAGAAGCCTGCATGGCACCAAGTACAAAGTGGG |
| | ACCAATCTGCTCTGTCATCTACCAAGCCAGTGGAGGAAGCATTGACTGGTCCTATGATTATGGCATC |
| | AAGTACTCATTTGCCTTTGAACTGAGAGACACAGGGCGCTACGGCTTCCTCTTGCCAGCCCGTCAGA |
| | TCCTGCCCACAGCCGAGGAGACCTGGCTTGGCTTGAAGGCAATCATGGAGCATGTGCGAGACCACCC |
| | CTATTAG |

| | | |
|---|---|---|
| ORF Start: at 3 | | ORF Stop: TAG at 1278 |
| SEQ ID NO: 232 | 425 aa | MW at 47808.7 kD |

| | |
|---|---|
| NOV27d, | TMGHHHHHHRLILFFGALFGHIYCLETFVGDQVLEIVPSNEEQIKNLLQLEAQEHLQLDFWKSPTTP |
| 183852323 | |
| Protein Sequence | GETAHVRVPFVNVQAVKVFLESQGIAYSIMIEDVQVLLDKENEEMLFNRRRERSGNFNFGAYHTLEE |
| | ISQEMDNLVAEHPGLVSKVNIGSSFENRPMNVLKFSTGGDKPAIWLDAGIHAREWVTQATALWTANK |
| | IVSDYGKDPSITSILDALDIFLLPVTNPDGYVFSQTKNRMWRKTRSKVSGSLCVGVDPNRNWDAGFG |
| | GPGASSNPCSDSYHGPSANSEVEVKSIVDFIKSHGKVKAFITLHSYSQLLMFPYGYKCTKLDDFDEL |
| | SEVAQKAAQSLRSLHGTKYKVGPICSVIYQASGGSIDWSYDYGIKYSFAFELRDTGRYGFLLPARQI |
| | LPTAEETWLGLKAIMEHVRDHPY |

| | |
|---|---|
| SEQ ID NO: 233 | 1259bp |

| | |
|---|---|
| NOV27e, | CCACCATGAGGTTGATCCTGTTTTTTGGTGCCCTTTTTGGGCATATCTACTGTCTAGAAACATTTGT |
| 173229182 DNA | |
| Sequence | GGGAGACCAAGTTCTTGAGATTGTACCAAGCAATGAAGAACAAATTAAAAATCTGCTACAATTGGAC |
| | GCTCAAGAACATCTCCAGCTTGATTTTTGGAAATCACCCACCACCCCAGGGGAGACAGCCCACGTCC |
| | GAGTTCCCTTCGTCAACGTCCAGGCAGTCAAAGTGTTCTTGGAGTCCCAGGGAATTGCCTATTCCAT |
| | CATGATTGAAGACGTGCAGGTCCTGTTGGACAAAGAGAATGAAGAAATGCTTTTTAATAGGAGAAGA |
| | GAACGGAGTGGTAACTTCAATTTTGGGGCCTACCATACCCTGGAAGAGATTTCCCAAGAAATGGATA |
| | ACCTCGTGGCTGAGCACCCTGGTCTAGTGAGCAAAGTGAATATTGGCTCTTCTTTTGAGAACCGGCC |
| | TATGAACGTGCTCAAGTTCAGCACCGGAGGAGACAAGCCAGCTATCTGGCTGGATGCTGGGATCCAT |
| | GCTCGAGAGTGGGTTACACAAGCTACGGCACTTTGGACAGCAAATAAGATTGTTTCTGATTATGGAA |
| | AGGACCCATCCATCACTTCCATTCTGGACGCCCTGGATATCTTCCTCCTGCCAGTCACAAACCCTGA |
| | TGGATACGTGTTCTCTCAAACCAAAAATCGTATGTGGCGGAAGACCCGGTCCAAGGTATCTGGAAGC |
| | CTCTGTGTTGGTGTGGATCCTAACCGGAACTGGGATGCAGGTTTTGGAGGACCTGGAGCCAGCAGCA |
| | ACCCTTGCTCTGATTCATACCACGGACCCAGTGCCAACTCTGAAGTTGAAGTGAAATCCATAGTGGA |
| | CTTCATCAAGAGTCATGGAAAAGTCAAGGCCTTCATTACCCTCCACAGCTATTCCCAGCTGCTGATG |
| | TTCCCCTATGGGTACAAATGTACCAAGTTAGATGACTTTGATGAGCTGAGTGAAGTGGCCCAAAAGG |
| | CTGCCCAATCTCTGAGAAGCCTGCATGGCACCAAGTACAAAGTGGGACCAATCTGCTCTGTCATCTA |
| | CCAAGCCAGTGGAGGAAGCATTGACTGGTCCTATGATTATGGCATCAAGTACTCATTTGCCTTTGAA |
| | CTGAGAGACACAGGGCGCTACGGCTTCCTCTTGCCAGCCCGTCAGATCCTGCCCACAGCCGAGGAGA |
| | CCTGGCTTGGCTTGAAGGCAATCATGGAGCATGTGCGAGACCACCCCTATTAG |

| | | |
|---|---|---|
| ORF Start: at 3 | | ORF Stop: TAG at 1257 |
| SEQ ID NO: 234 | 418 aa | MW at 46928.8 kD |

| | |
|---|---|
| NOV27e, | TMRLILFFGALFGHIYCLETFVGDQVLEIVPSNEEQIKNLLQLEAQEHLQLDFWKSPTTPGETAHVR |
| 173229182 | |
| Protein Sequence | VPFVNVQAVKVFLESQGIAYSIMIEDVQVLLDKENEEMLFNRRRERSGNFNFGAYHTLEEISQEMDN |
| | LVAEHPGLVSKVNIGSSFENRPMNVLKFSTGGDKPAIWLDAGIHAREWVTQATALWTANKIVSDYGK |
| | DPSITSILDALDIFLLPVTNPDGYVFSQTKNRMWRKTRSKVSGSLCVGVDPNRNWDAGFGGPGASSN |
| | PCSDSYHGPSANSEVEVKSIVDFIKSHGKVKAFITLHSYSQLLMFPYGYKCTKLDDFDELSEVAQKA |
| | AQSLRSLHGTKYKVGPICSVIYQASGGSIDWSYDYGIKYSFAFELRDTGRYGFLLPARQILPTAEET |
| | WLGLKAIMEHVRDHPY |

| | |
|---|---|
| SEQ ID NO: 235 | 1277 bp |

| | |
|---|---|
| NOV27f, | CCACCATGAGGTTGATCCTGTTTTTTGGTGCCCTTTTTGGGCATATCTACTGTCTAGAAACATTTGT |
| 173172465 | |
| DNA Sequence | GGGAGACCAAGTTCTTGAGATTGTACCAAGCAATGAAGAACAAATTAAAAATCTGCTACAATTGGAG |
| | GCTCAAGAACATCTCCAGCTTGATTTTTGGAAATCACCCACCACCCCAGGGGAGACAGCCCACGTCC |
| | GAGTTCCCTTCGTCAACGTCCAGGCAGTCAAAGTGTTCTTGGAGTCCCAGGGAATTGCCTATTCCAT |
| | CATGATTGAAGACGTGCAGGTCCTGTTGGACAAAGAGAATGAAGAAATGCTTTTTAATAGGAGAAGA |
| | GAACGGAGTGGTAACTTCAATTTTGGGGCCTACCATACCCTGGAAGAGATTTCCCAAGAAATGGATA |
| | ACCTCGTGGCTGAGCACCCTGGTCTAGTGAGCAAAGTGAATATTGGCTCTTCTTTTGAGAACCGGCC |
| | TATGAACGTGCTCAAGTTCAGCACCGGAGGAGACAAGCCAGCTATCTGGCTGGATGCTGGGATCCAT |
| | GCTCGAGAGTGGGTTACACAAGCTACGGCACTTTGGACAGCAAATAAGATTGTTTCTGATTATGGAA |
| | AGGACCCATCCATCACTTCCATTCTGGACGCCCTGGATATCTTCCTCCTGCCAGTCACAAACCCTGA |
| | TGGATACGTGTTCTCTCAAACCAAAAATCGTATGTGGCGGAAGACCCGGTCCAAGGTATCTGGAAGC |
| | CTCTGTGTTGGTGTGGATCCTAACCGGAACTGGGATGCAGGTTTTGGAGGACCTGGAGCCAGCAGCA |
| | ACCCTTGCTCTGATTCATACCACGGACCCAGTGCCAACTCTGAAGTTGAAGTGAAATCCATAGTGGA |
| | CTTCATCAAGAGTCATGGAAAAGTCAAGGCCTTCATTACCCTCCACAGCTATTCCCAGCTGCTGATG |
| | TTCCCCTATGGGTACAAATGTACCAAGTTAGATGACTTTGATGAGCTGAGTGAAGTGGCCCAAAAGG |
| | CTGCCCAATCTCTGAGAAGCCTGCATGGCACCAAGTACAAAGTGGGACCAATCTGCTCTGTCATCTA |
| | CCAAGCCAGTGGAGGAAGCATTGACTGGTCCTATGATTATGGCATCAAGTACTCATTTGCCTTTGAA |
| | CTGAGAGACACAGGGCGCTACGGCTTCCTCTTGCCAGCCCGTCAGATCCTGCCCACAGCCGAGGAGA |
| | CCTGGCTTGGCTTGAAGGCAATCATGGAGCATGTGCGAGACCACCCCTATCACCATCACCACCATCA |
| | CTAG |

| | | |
|---|---|---|
| ORF Start: at 3 | | ORF Stop: TAG at 1275 |
| SEQ ID NO: 236 | 4424 aa | MW at 47751.6 kD |

| | |
|---|---|
| NOV27f, | TMRLILFFGALFGHIYCLETFVGDQVLEIVPSNEEQIKNLLQLEAQEHLQLDFWKSPTTPGETAHVR |
| 173172465 | |
| Protein Sequence | VPFVNVQAVKVFLESQGIAYSIMIEDVQVLLDKENEEMLFNRRRERSGNFNFGAYHTLEEISQEMDN |
| | LVAEHPGLVSKVNIGSSFENRPMNVLKFSTGGDKPAIWLDAGIHAREWVTQATALWTANKIVSDYGK |
| | DPSITSILDALDIFLLPVTNPDGYVFSQTKNRMWRKTRSKVSGSLCVGVDPNRNWDAGFGGPGASSN |
| | PCSDSYHGPSANSEVEVKSIVDFIKSHGKVKAFITLHSYSQLLMFPYGYKCTKLDDFDELSEVAQKA |
| | AQSLRSLHGTKYKVGPICSVIYQASGGSIDWSYDYGIKYSFAFELRDTGRYGFLLPARQILPTAEET |
| | WLGLKAIMEHVRDHPYHHHHHH |

| | |
|---|---|
| SEQ ID NO: 237 | 1269 bp |

| | |
|---|---|
| NOV27g, | GGGCATATCTACTGTCTAGAAACATTTGTGGGAGACCAAGTTCTTGAGATTGTACCAAGCAATGAAG |
| CG56246-01 | |
| DNA Sequence | AACAAATTAAAAATCTGCTACAATTGGAGGCTCAAGAACATCTCCAGCTTGATTTTTCGAAATCACC |
| | CACCACCCCAGCGGAGACAGCCCACGTCCGAGTTCCCTTCGTCAACGTCCAGGCAGTCAAAGTGTTC |
| | TTGGAGTCCCAGGGAATTGCCTATTCCATCATGATTGAAGACGTGCAGGTCCTGTTGGACAAAGAGA |
| | ATGAAGAAATGCTTTTTAATAGGAGAAGAGAACGGAGTGGTAACTTCAATTTTGGGGCCTACCATAC |
| | CCTGGAAGAGATTTCCCAAGAAATGGATAACCTCGTGGCTGAGCACCCTGGTCTAGTGACCAAAGTG |
| | AATATTGGCTCTTCTTTTGAGAACCCGCCTATGAACGTCCTCAAGTTCAGCACCCGAGGAGACAAGC |
| | CAGCTATCTGGCTGGATGCTGGGATCCATGCTCGAGAGTGGGTTACACAAGCTACGGCACTTTGGAC |
| | AGCAAATAAGATTGTTTCTGATTATGGAAAGGACCCATCCATCACTTCCATTCTGGACGCCCTGGAT |
| | ATCTTCCTCCTGCCAGTCACAAACCCTGATCGATACGTGTTCTCTCAAACCAAAAATCGTATGTGGC |
| | GGAAGACCCGGTCCAAGGTATCTGGAAGCCTCTGTGTTGGTGTCGATCCTAACCGGAACTGGGATGC |
| | AGGTTTTGGAGGACCTGGAGCCAGCAGCAACCCTTGCTCTGATTCATACCACCGACCCAGTGCCAAC |
| | TCTGAAGTTGAAGTGAAATCCATAGTGGACTTCATCAAGAGTCATCGAAAAGTCAACGCCTTCATTA |
| | CCCTCCACAGCTATTCCCAGCTGCTGATGTTCCCCTATCGGTACAAATGTACCAAGTTAGATGACTT |
| | TGATGAGCTGAGTGAAGTGGCCCAAAAGGCTGCCCAATCTCTGAGAAGCCTGCATGGCACCAAGTAC |
| | AAAGTGGGACCAATCTGCTCTGTCATCTACCAAGCCAGTGGAGGAAGCATTGACTGGTCCTATGATT |
| | ATGGCATCAAGTACTCATTTGCCTTTGAACTGAGAGACACAGGGCCCTACGGCTTCCTCTTGCCAGC |
| | CCGTCAGATCCTGCCCACAGCCGAGGAGACCTGGCTTGGCTTGAAGGCAATCATGGAGCATGTGCGA |
| | GACCACCCCTATTAGGGCCCTGGGGAAGAAACAAGAGCCATTAAAATCTCTTTCGTTTGAAGC |

| | | |
|---|---|---|
| ORF Start: at 1 | | ORF Stop: TAG at 1219 |
| SEQ ID NO: 238 | 406aa | MW at 45518.0 kD |

| | |
|---|---|
| NOV27g, | GHIYCLETFVGDQVLEIVPSNEEQIKNLLQLEAQEHLQLDFWKSPTTPGETAHVRVPFVNVQAVKVF |
| CG56246-01 | |
| Protein Sequence | LESQGIAYSIMIEDVQVLLDKENEEMLFNRRRERSGNFNFGAYHTLEEISQEMDNLVAEHPGLVSKV |
| | NIGSSFENRPMNVLKFSTGGDKPAIWLDAGIHAREWVTQATALWTANKIVSDYGKDPSITSILDALD |
| | IFLLPVTNPDGYVFSQTKNRMWRKTRSKVSGSLCVGVDPNRNWDAGFGGPGASSNPCSDSYHGPSAN |
| | SEVEVKSIVDFIKSHGKVKAFITLHSYSQLLMFPYGYKCTKLDDFDELSEVAQKAAQSLRSLHGTKY |
| | KVGPICSVIYQASGGSIDWSYDYGIKYSFAFELRDTGRYGFLLPARQILPTAEETWLGLKAIMEHVR |
| | DHPY |

| | |
|---|---|
| SEQ ID NO: 239 | 253 bp |

| | |
|---|---|
| NOV27h, | CACCAGATCTCAAGTTCTTGAGATTGTACCAAGCAATGAAGAACAAATTAAAAATCTGCTACAATTG |
| 274057795 DNA | |
| Sequence | GAGGCTCAAGAACATCTCCAGCTTGATTTTTGGAAATCACCCACCACCCCAGGGGAGACAGCCCACG |
| | TCCGAGTTCCCTTCGTCAACGTCCAGGCAGTCAAAGTGTTCTTGGAGTCCCAGGGAATTGCCTATTC |
| | CATCATGATTGAAGACGTGCAGGTCCTGTTGGACAAAGAGAATGTCGACGGC |

| | | |
|---|---|---|
| ORF Start: at 2 | | ORF Stop: end of sequence |
| SEQ ID NO: 240 | 84 aa | MW at 9501.7 kD |

| | |
|---|---|
| NOV27h, | TRSQVLEIVPSNEEQIKNLLQLEAQEHLQLDFWKSPTTPGETAHVRVPFVNVQAVKVFLESQGIAYS |
| 274057795 | |
| Protein Sequence | IMIEDVQVLLDKENVDG |

| | |
|---|---|
| SEQ ID NO: 241 | 1243 bp |

| | |
|---|---|
| NOV27i, | CACCAGATCTCCCACCGGGCATATCTACTGTCTAGAAACATTTGTGGGAGACCAAGTTCTTGAGATT |
| 274057823 | |
| DNA Sequence | GTACCAAGCAATGAAGAACAAATTAAAAATCTGCTACAATTGGAGGCTCAAGAACATCTCCAGCTTG |
| | ATTTTTGGAAATCACCCACCACCCCAGGGGAGACAGCCCACGTCCGAGTTCCCTTCGTCAACGTCCA |
| | GGCAGTCAAAGTGTTCTTGGAGTCCCAGGGAATTGCCTATTCCATCATGATTGAAGACGTGCAGGTC |
| | CTGTTGGACAAAGAGAATGAAGAAATGCTTTTTAATAGGAGAAGAGAACGGAGTGGTAACTTCAATT |
| | TTGGGGCCTACCATACCCTGGAAGAGATTTCCCAAGAAATGGATAACCTCGTGGCTGAGCACCCTGG |
| | TCTAGTGAGCAAAGTGAATATTGGCTCTTCTTTTGAGAACCGGCCTATGAACGTGCTCAAGTTCAGC |
| | ACCGGAGGAGACAAGCCAGCTATCTGGCTGGATGCTGGGATCCATGCTCGAGAGTGGGTTACACAAG |
| | CTACGACACTTTGGACAGCAAATAAGATTGTTTCTGATTATGGAAAGGACCCATCCATCACTTCCAT |
| | TCTGAACGCCCTGGATATCTTCCTCCTGCCAGTCACAAACCCTGATGGATACGTGTTCTCTCAAACC |
| | AAAAATCGTATGTGGCGGAAGACCCGGTCCAAGGTATCTGGAAGCCTCTGTGTTGGTGTGGATCCTA |
| | ACCGGAACTGGGATGCAGGTTTTGGAGGACCTGGAGCCAGCAGCAACCCTTGCTCTGATTCATACCA |
| | CGGACCCAGTGCCAACTCTGAAGTTGAAGTGAAATCCATAGTGGACTTCATCAAGAGTCATGGAAAA |
| | GTCAAGGCCTTCATTACCCTCCACAGCTATTCCCAGCTGCTGATGTTCCCCTATGGGTACAAATGTA |
| | CCAAGTTAGATGACTTTGATGAGCTGAGTGAAGTGGCCCAAAAGGCTGCCCAATCTCTGAGAAGCCT |
| | GCATGGCACCAAGTACAAAGTGGGACCAATCTGCTCTGTCATCTACCAAGCCAGTGGAGGAAGCATT |
| | GACTGGTCCTATGATTATGGCATCAAGTACTCATTTGCCTTTGAACTGAGAGACACAGGGCGCTACG |
| | GCTTCCTCTTGCCAGCCCGTCAGATCCTGCCCACAGCCGAGGAGACCTGGCTTGGCTTGAAGGCAAT |
| | CATGGAGCATGTGCCAGACCACCCCTATGTCGACGGC |

| | | |
|---|---|---|
| ORF Start: at 2 | | ORF Stop: end of sequence |
| SEQ ID NO: 242 | 414 aa | MW at 46361.9 kD |

| | |
|---|---|
| NOV27i, | TRSPTGHIYCLETFVGDQVLEIVPSNEEQIKNLLQLEAQEHLQLDFWKSPTTPGETAHVRVPFVNVQ |
| 274057823 | AVKVFLESQGIAYSIMIEDVQVLLDKENEEMLFNRRRERSGNFNFGAYHTLEEISQEMDNLVAEHPG |
| Protein | |
| Sequence | LVSKVNIGSSFENRPMNVLKFSTGGDKPAIWLDAGIHAREWVTQATALWTANKIVSDYGKDPSITSI |
| | LDALDIFLLPVTNPDGYVFSQTKNRMWRKTRSKVSGSLCVGVDPNRNWDAGFGGPGASSNPCSDSYH |
| | GPSANSEVEVKSIVDFIKSHGKVKAFITLHSYSQLLMFPYGYKCTKLDDFDELSEVAQKAAQSLRSL |
| | HGTKYKVGPICSVIYQASGGSIDWSYDYGIKYSFAFELRDTGRYGFLLPARQILPTAEETWLGLKAI |
| | MEHVRDHPYVDG |

| | |
|---|---|
| SEQ ID NO: 243 | 1243 bp |

| | |
|---|---|
| NOV27j, CACCAGATCTCCCACCGGGCATATCTACTGTCTAGAAACATTTGTGGGAGACCAAGTTCTTGAGATT | |
| 274057830 | |
| DNA Sequence | GTACCAAGCAATCAAGAACAAATTAAAAATCTGCTACAATTGGAGGCTCAAGAACATCTCCAGCTTG |
| | ATTTTTGGAAATCACCCACCACCCCAGGGGAGACAGCCCACGTCCGAGTTCCCTTCGTCAACGTCCA |
| | GGCAGTCAAAGTGTTCTTGGAGTCCCAGGGAATTGCCTATTCCATCATGATTGAAGACGTGCAGGTC |
| | CTGTTGGACAAAGAGAATGAAGAAATGCTTTTTAATAGGAGAAGAGAACGGAGTGGTAACTTCAATT |
| | TTGGGGCCTACCATACCCTGGAAGAGATTTCCCAAGAAATGGATAACCTCGTGGCTCAGCACCCTGG |
| | TCTAGTCAGCAAAGTGAATATTGGCTCTTCTTTTGAGAACCGGCCTATGAACGTCCTCAAGTTCAGC |
| | ACCCGAGGAGACAAGCCAGCTATCTGGCTGGATGCTCGGATCCATGCTCGAGAGTGGGTTACACAAG |
| | CTACGGCACTTTGGACAGCAAATAAGATTGTTTCTGATTATGGAAAGGACCCATCCATCACTTCCAT |
| | TCTGGACGCCCTGGATATCTTCCTCCTCCCAGTCACAAACCCTGATGGATACGTGTTCTCTCAAACC |
| | AAAAATCGTATGTGGCGGAAGACCCGGTCCAAGGTATCTCGAAGCCTCTGTGTTGGTGTGGATCCTA |
| | ACCGGAACTGGGATGCACGTTTTGGAGGACCTGGAGCCAGCAGCAACCCTTGCTCTGATTCATACCA |
| | CGGACCCAGTGCCAACTCTGAAGTTGAAGTGAAATCCATAGTGGACTTCATCAAGAGTCATGGAAAA |
| | GTCAAGGCCTTCATTACCCTCCACAGCTATTCCCAGCTGCTGATGTTCCCCTATGGGTACAAATGTA |
| | CCAAGTTAGATGACTTTGATGAGCTGAGTGAAGTGGCCCAAAAGGCTGCCCAATCTCTGAGAAGCCT |
| | GCATGGCACCAAGTACAAAGTGGGACCAATCTGCTCTGTCATCTACCAAGCCAGTGGAGGAAGCATT |
| | GACTGGTCCTATGATTATGGCATCAAGTACTCATTTGCCTTTGAACTGAGAGACACAGGGCGCTACG |
| | GCTTCCTCTTGCCAGCCCGTCACATCCTGCCCACAGCCGAGGAGACCTGGCTTGGCTTGAAGGCAAT |
| | CATGGAGCATGTGCGAGACCACCCCTATGTCGACGGC |

| | | |
|---|---|---|
| ORF Start: at 2 | | ORF Stop: end of sequence |
| SEQ ID NO: 244 | 414 aa | MW at 46331.9 kD |

| | |
|---|---|
| NOV27j, | TRSPTGHIYCLETFVGDQVLEIVPSNEEQIKNLLQLEAQEHLQLDFWKSPTTPGETAHVRVPFVNVQ |
| 274057830 | |
| Protein | AVKVFLESQGIAYSIMIEDVQVLLDKENEEMLFNRRRERSGNFNFGAYHTLEEISQEMDNLVAEHPG |
| Sequence | |
| | LVSKVNIGSSFENRPMNVLKFSTGGDKPAIWLDAGIHAREWVTQATALWTANKIVSDYGKDPSITSI |
| | LDALDIFLLPVTNPDGYVFSQTKNRMWRKTRSKVSGSLCVGVDPNRNWDAGFGGPGASSNPCSDSYH |
| | GPSANSEVEVKSIVDFIKSHGKVKAFITLHSYSQLLMFPYGYKCTKLDDFDELSEVAQKAAQSLRSL |
| | HGTKYKVGPICSVIYQASGGSIDWSYDYGIKYSFAFELRDTGRYGFLLPARQILPTAEETWLGLKAI |
| | MEHVRDHPYVDG |

| | |
|---|---|
| SEQ ID NO: 245 | 859 bp |

| | |
|---|---|
| NOV27k, | CACCAGATCTTACCATACCCTGGAAGAGATTTCCCAAGAAATGGATAACCTCGTGGCTGAGCACCCT |
| 274057838 DNA | |
| Sequence | GGTCTAGTGAGCAAAGTGAATATTGGCTCTTCTTTTGAGAACCGGCCTATGAACGTGCTCAAGTTCA |
| | GCACCGGAGGAGACAAGCCAGCTATCTGGCTGGATGCTGGGATCCATGCTCGAGAGTGGGTTACACA |
| | AGCTACGACACTTTGGACAGCAAATAAOATTGTTTCTGATTATCGAAAGCACCCATCCATCACTTCC |
| | ATTCTGGACGCCCTGGATATCTTCCTCCTGCCAGTCACAAACCCTGATGGATACGTGTTCTCTCAAA |
| | CCAAAAATCGTATGTGGCGGAAGACCCGGTCCAAGGTATCTGGAAGCCTCTGTGTTGGTGTGGATCC |
| | TAACCGGAACTGGGATGCAGGTTTTGGAGGACCTGGAGCCAGCAGCAACCCTTGCTCTGATTCATAC |
| | CACGGACCCAGTGCCAACTCTGAAGTTGAAGTGAAATCCATAGTGGACTTCATCAAGAGTCATGGAA |
| | AAGTCAAGGCCTTCATTACCCTCCACAGCTATTCCCAGCTGCTGATGTTCCCCTATGGGTACAAATG |
| | TACCAAGTTAGATGACTTTGATGAGCTGAGTGAAGTGGCCCAAAAGGCTGCCCAATCTCTGAGAAGC |
| | CTGCATGGCACCAAGTACAAAGTGGGACCAATCTGCTCTGTCATCTACCAAGCCAGTGGAGGAAGCA |
| | TTGACTGGTCCTATGATTATGGCATCAAGTACTCATTTGCCTTTGAACTGAGAGACACAGGGCGCTA |
| | CGGCTTCCTCTTGCCAGCCCGTCAGATCCTGCCCACAGCCGAGGAGGTCGACGGC |

| | | |
|---|---|---|
| ORF Start: at 2 | | ORF Stop: end of sequence |
| SEQ ID NO: 246 | 286 aa | Mw at 31599.2 kD |

| | |
|---|---|
| NOV27k, | TRSYHTLEEISQEMDNLVAEHPGLVSKVNIGSSFENRPMNVLKFSTGGDKPAIWLDAGIHAREWVTQ |
| 274057838 | |
| Protein Sequence | ATTLWTANKIVSDYGKDPSITSILDALDIFLLPVTNPDGYVFSQTKNRMWRKTRSKVSGSLCVGVDP |
| | NRNWDAGFGGPGASSNPCSDSYHGPSANSEVEVKSIVDFIKSHGKVKAFITLHSYSQLLMFPYGYKC |
| | TKLDDFDELSEVAQKAAQSLRSLHGTKYKVGPICSVIYQASGGSIDWSYDYGIKYSFAFELRDTGRY |
| | GFLLPARQILPTAEEVDG |

| | |
|---|---|
| SEQ ID NO: 247 | 1258 bp |

| | |
|---|---|
| NOV27l, | CCACCATGAGGTTGATCCTGTTTTTTGGTGCCCTTTTTGGGCATATCTACTGTCTAGAAACATTTGT |
| CG56246-03 | |
| DNA Sequence | GGGAGACCAAGTTCTTGAGATTGTACCAAGCAATGAAGAACAAATTAAAAATCTGCTACAATTGGAG |
| | GCTCAAGAACATCTCCAGCTTGATTTTTGGAAATCACCCACCACCCCAGGGGAGACAGCCCACGTCC |
| | GAGTTCCCTTCGTCAACGTCCAGGCAGTCAAAGTGTTCTTGGAGTCCCAGGGAATTGCCTATTCCAT |
| | CATGATTGAAGACGTGCAGGTCCTGTTGGACAAAGAGAATGAAGAAATGCTTTTTAATAGGAGAAGA |
| | GAACGGAGTGGTAACTTCAATTTTGGGGCCTACCATACCCTGGAAGAGATTTCCCAAGAAATGGATA |
| | ACCTCGTGGCTGAGCACCCTGGTCTAGTGAGCAAAGTGAATATTGGCTCTTCTTTTGAGAACCGGCC |
| | TATGAACGTGCTCAAGTTCAGCACCGGAGGAGACAAGCCAGCTATCTGGCTGGATGCTGGGATCCAT |
| | GCTCGAGAGTGGGTTACACAAGCTACGGCACTTTGGACAGCAAATAAGATTGTTTCTGATTATGGAA |
| | AGGACCCATCCATCACTTCCATTCTGGACGCCCTGGATATCTTCCTCCTGCCAGTCACAAACCCTGA |
| | TGGATACGTGTTCTCTCAAACCAAAAATCGTATGTGGCGGAAGACCCGGTCCAAGGTATCTGGAAGC |
| | CTCTGTGTTGGTGTGGATCCTAACCGGAACTGGGATGCAGGTTTTGGAGGACCTGGAGCCAGCAGCA |
| | ACCCTTGCTCTGATTCATACCACGGACCCAGTGCCAACTCTGAAGTTGAAGTGAAATCCATAGTGGA |
| | CTTCATCAAGAGTCATGGAAAAGTCAAGGCCTTCATTACCCTCCACAGCTATTCCCAGCTGCTGATG |
| | TTCCCCTATGGGTACAAATGTACCAAGTTAGATGACTTTGATGAGCTGAGTGAAGTGGCCCAAAAGG |
| | CTGCCCAATCTCTGAGAAGCCTGCATGGCACCAAGTACAAAGTGGGACCAATCTGCTCTGTCATCTA |
| | CCAAGCCAGTGGAGGAAGCATTGACTGGTCCTATGATTATGGCATCAAGTACTCATTTGCCTTTGAA |
| | CTGAGAGACACAGGGCGCTACGGCTTCCTCTTGCCAGCCCGTCAGATCCTGCCCACAGCCGAGGAGA |
| | CCTGGCTTGGCTTGAAGGCAATCATGGAGCATGTGCGAGACCACCCCTATTA |

| | | |
|---|---|---|
| ORF Start: at 3 | | ORF Stop: at 1257 |
| SEQ ID NO: 248 | 418 aa | MW at 46928.8 kD |

| | |
|---|---|
| N0V27l, | TMRLILFFGALFGHIYCLETFVGDQVLEIVPSNEEQIKNLLQLEAQEHLQLDFWKSPTTPGETAHVR |
| CG56246-03 | |
| Protein | VPFVNVQAVKVFLESQGIAYSIMIEDVQVLLDKENEEMLFNRRRERSGNFNFGAYHTLEEISQEMDN |
| Sequence | |
| | LVAEHPGLVSKVNIGSSFENRPMNVLKFSTGGDKPAIWLDAGIHAREWVTQATALWTANKIVSDYGK |
| | DPSITSILDALDIFLLPVTNPDGYVFSQTKNRMWRKTRSKVSGSLCVGVDPNRNWDAGFGGPGASSN |
| | PCSDSYHGPSANSEVEVKSIVDFIKSHGKVKAFITLHSYSQLLMFPYGYKCTKLDDFDELSEVAQKA |
| | AQSLRSLHGTKYKVGPICSVIYQASGGSIDWSYDYGIKYSFAFELRDTGRYGFLLPARQILPTAEET |
| | WLGLKAIMEHVRDHPY |

| | |
|---|---|
| SEQ ID NO: 249 | 1276 bp |

| | |
|---|---|
| NOV27m, | CCACCATGAGGTTGATCCTGTTTTTTGGTGCCCTTTTTGGGCATATCTACTGTCTAGAAACATTTG |
| CG56246-05 | |
| DNA Sequence | TGGGAGACCAAGTTCTTGAGATTGTACCAAGCAATGAAGAACAAATTAAAAATCTGCTACAATTGG |
| | AGGCTCAAGAACATCTCCAGCTTGATTTTTGGAAATCACCCACCACCCCAGGGGAGACAGCCCACG |
| | TCCGAGTTCCCTTCGTCAACGTCCAGGCAGTCAAAGTGTTCTTGGAGTCCCAGGGAATTCCCTATT |
| | CCATCATGATTGAAGACGTGCAGGTCCTGTTGGACAAAGAGAATGAAGAAATGCTTTTTAATAGGA |
| | GAAGAGAACGGAGTGGTAACTTCAATTTTGGGGCCTACCATACCCTGGAAGAGATTTCCCAAGAAA |
| | TGGATAACCTCGTGGCTGAGCACCCTGGTCTAGTGAGCAAAGTGAATATTGGCTCTTCTTTTGAGA |
| | ACCCGCCTATGAACGTGCTCAAGTTCAGCACCGGAGGAGACAAGCCAGCTATCTGGCTGGATGCTG |
| | GGATCCATGCTCGAGAGTGGGTTACACAAGCTACGGCACTTTGGACAGCAAATAAGATTGTTTCTG |
| | ATTATGGAAAGGACCCATCCATCACTTCCATTCTGGACGCCCTGGATATCTTCCTCCTGCCAGTCA |
| | CAAACCCTGATGGATACGTGTTCTCTCAAACCAAAAATCGTATGTGGCGGAAGACCCGGTCCAAGG |
| | TATCTGGAAGCCTCTGTGTTGGTGTGGATCCTAACCGGAACTGGGATGCAGGTTTTGGAGGACCTG |
| | GAGCCAGCAGCAACCCTTGCTCTGATTCATACCACGGACCCAGTGCCAACTCTGAAGTTGAAGTGA |
| | AATCCATAGTGGACTTCATCAAGAGTCATGGAAAAGTCAAGGCCTTCATTACCCTCCACAGCTATT |
| | CCCAGCTGCTGATGTTCCCCTATGGGTACAAATGTACCAAGTTAGATGACTTTGATGAGCTGAGTG |
| | AAGTGGCCCAAAAGGCTGCCCAATCTCTGAGAAGCCTGCATGGCACCAAGTACAAAGTGGGACCAA |
| | TCTGCTCTGTCATCTACCAAGCCAGTGGAGGAAGCATTGACTGGTCCTATGATTATGGCATCAAGT |
| | ACTCATTTGCCTTTGAACTGAGAGACACAGGGCGCTACGGCTTCCTCTTGCCAGCCCGTCAGATCC |
| | TGCCCACAGCCGAGGAGACCTGGCTTGGCTTGAAGGCAATCATGGAGCATGTGCGAGACCACCCCT |
| | ATCACCATCACCACCATCACTA |

| | | |
|---|---|---|
| ORF Start: ATG at 6 | | ORF Stop: at 1257 |
| SEQ ID NO: 250 | 417 aa | MW at 46827.6 kD |

| | |
|---|---|
| NOV27m, | MRLILFFGALFGHIYCLETFVGDQVLEIVPSNEEQIKNLLQLEAQEHLQLDFWKSPTTPGETAHVR |
| CG56246-05 | |
| Protein Sequence | VPFVNVQAVKVFLESQGIAYSIMIEDVQVLLDKENEEMLFNRRRERSGNFNFGAYHTLEEISQEMD |
| | NLVAEHPGLVSKVNIGSSFENRPMNVLKFSTGGDKPAIWLDAGIHAREWVTQATALWTANKIVSDY |
| | GKDPSITSILDALDIFLLPVTNPDGYVFSQTKNRMWRKTRSKVSGSLCVGVDPNRNWDAGFGGPGA |
| | SSNPCSDSYHGPSANSEVEVKSIVDFIKSHGKVKAFITLHSYSQLLMFPYGYKCTKLDDFDELSEV |
| | AQKAAQSLRSLHGTKYKVGPICSVIYQASGGSIDWSYDYGIKYSFAFELRDTGRYGFLLPARQILP |
| | TAEETWLGLKAIMEHVRDHPY |

Sequence comparison of the above protein sequences yields the following sequence relationships shown in Table 27B.

TABLE 27B
Comparison of NOV27a against NOV27b through NOV27m.

| | NOV27a Residues/ | Identities/Similarities |
|---|---|---|
| Protein Sequence | Match Residues | for the Matched Region |
| NOV27b | 10 . . . 425 | 415/416 (99%) |
| | 2 . . . 417 | 415/416 (99%) |
| NOV27c | 10 . . . 425 | 416/416 (100%) |
| | 3 . . . 418 | 416/416 (100%) |
| NOV27d | 1 . . . 425 | 425/425 (100%) |
| | 1 . . . 425 | 425/425 (100%) |
| NOV27e | 10 . . . 425 | 416/416 (100%) |
| | 3 . . . 418 | 416/416 (100%) |
| NOV27f | 10 . . . 425 | 416/416 (100%) |
| | 3 . . . 418 | 416/416 (100%) |
| NOV27g | 20 . . . 425 | 406/406 (100%) |
| | 1 . . . 406 | 406/406 (100%) |
| NOV27h | 32 . . . 109 | 78/78 (100%) |
| | 4 . . . 81 | 78/78 (100%) |
| NOV27i | 20 . . . 425 | 405/406 (99%) |
| | 6 . . . 411 | 405/406 (99%) |
| NOV27j | 20 . . . 425 | 406/406 (100%) |
| | 6 . . . 411 | 406/406 (100%) |
| NOV27k | 128 . . . 408 | 279/281 (99%) |
| | 3 . . . 283 | 280/281 (99%) |
| NOV27l | 10 . . . 425 | 416/416 (100%) |
| | 3 . . . 418 | 416/416 (100%) |
| NOV27m | 10 . . . 425 | 416/416 (100%) |
| | 2 . . . 417 | 416/416 (100%) |

Further analysis of the NOV27a protein yielded the following properties shown in Table 27C.

TABLE 27C
Protein Sequence Properties NOV27a

| SignalP | | |
|---|---|---|
| analysis: | Cleavage site between residues 25 and 26 | |
| PSORT II | PSG: | a new signal peptide prediction method |
| analysis: | | N-region: length 10; pos. chg 1; neg. chg 0 |
| | | H-region: length 15; peak value 11.73 |
| | | PSG score: 7.33 |
| | GvH: | von Heijne's method for signal seq. recognition |
| | | GvH score (threshold: −2.1): −2.32 |
| | | possible cleavage site: between 24 and 25 |
| | >>> Seems to have no N-terminal signal peptide | |
| | ALOM: | Klein et al's method for TM region allocation |
| | | Init position for calculation: 1 |
| | | Tentative number of TMS(s) for the threshold 0.5: 1 |
| | | Number of TMS(s) for threshold 0.5: 0 |
| | | PERIPHERAL Likelihood = 0.53 (at 211) |
| | | ALOM score: −0.27 (number of TMSs: 0) |
| | MTOP: | Prediction of membrane topology (Hartmann et al.) |
| | | Center position for calculation: 6 |
| | | Charge difference: 0.0 C(3.0) - N(3.0) |
| | | N >= C: N-terminal side will be inside |

MITDISC: discrimination of mitochondrial targeting seq

| | | | |
|---|---|---|---|
| R content: | 1 | Hyd Moment | 2.28 |
| Hyd Moment (95): | 3.41 | (75): | |
| D/E content: | 1 | G content: | 3 |
| Score: | −6.06 | S/T content: | 1 |

| Gavel: | prediction of cleavage sites for mitochondrial preseq |
|---|---|
| | R-2 motif at 20 HRL\|IL |
| NUCDISC: discrimination of nuclear localization signals | |
| | pat4: none |
| | pat7: none |
| | bipartite: none |
| | content of basic residues: 9.4% |
| | NLS Score: −0.47 |
| KDEL: | ER retention motif in the C-terminus: none |
| ER Membrane Retention Signals: none | |
| SKL: | peroxisomal targeting signal in the C-terminus: none |
| PTS2: | 2nd peroxisomal targeting signal: none |
| VAC: | possible vacuolar targeting motif: none |
| RNA-binding motif: none | |
| Actinin-type actin-binding motif: | |
| | type 1: none |
| | type 2: none |
| NMYR: | N-myristoylation pattern : none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

-------------------------------------
Final Results (k = 9/23):

```
34.8%: mitochondrial
26.1%: cytoplasmic
13.0%: endoplasmic reticulum
  8.7%: extracellular, including cell wall
  8.7%: vacuolar
  8.7%: nuclear
```

>> prediction for CG56246-04 is mit (k = 23)

A search of the NOV27a protein against the Geneseq database, a proprietary database that contains sequences published in patents and patent publication, yielded several homologous proteins shown in Table 27D.

TABLE 27D
Geneseq Results for NOV27a

| Geneseq | Protein/Organism/Length | NOV27a Residues/ | Identities/Similarities | Expect |
|---|---|---|---|---|
| Identifier | [Patent #, Date] | Match Residues | for the Matched Region | Value |
| AAU87689 | Human pancreatic tumour | 10 . . . 425 | 415/416 (99%) | 0.0 |
| | protein #1 - Homo sapiens, | 2 . . . 417 | 415/416 (99%) | |
| | 417 aa. [WO200212331-A2, | | | |
| | 14-FEB-2002] | | | |
| AAW01505 | Wild-type human pancreatic | 10 . . . 425 | 415/416 (99%) | 0.0 |
| | carboxypeptidase 2 - Homo | 2 . . . 417 | 415/416 (99%) | |
| | sapiens, 417 aa. | | | |
| | [WO9513095-A2, | | | |
| | 18-MAY-1995] | | | |
| AAW01507 | Human pancreatic | 10 . . . 425 | 414/416 (99%) | 0.0 |
| | carboxypeptidase 2 variant | 2 . . . 417 | 414/416 (99%) | |
| | (T268G) - Synthetic, 417 aa. | | | |
| | [WO9513095-A2, | | | |
| | 18-MAY-1995] | | | |
| AAW01506 | Human pancreatic | 10 . . . 425 | 414/416 (99%) | 0.0 |
| | carboxypeptidase 2 variant | 2 . . . 417 | 414/416 (99%) | |
| | (A250G) - Synthetic, 417 aa. | | | |
| | [WO9513095-A2, | | | |
| | 18-MAY-1995] | | | |
| AAB54076 | Human pancreatic cancer | 10 . . . 425 | 412/416 (99%) | 0.0 |
| | antigen protein sequence | 13 . . . 428 | 412/416 (99%) | |
| | SEQ ID NO:528 - Homo | | | |
| | sapiens, 428 aa. | | | |
| | [WO200055320-A1, | | | |
| | 21-SEP-2000] | | | |

In a BLAST search of public sequence datbases, the NOV27a protein was found to have homology to the proteins shown in the BLASTP data in Table 27E.

TABLE 27E
Public BLASTP Results for NOV27a

| Protein | | | | |
|---|---|---|---|---|
| Accession | | NOV27a Residues/ | Identities/Similarities | Expect |
| Number | Protein/Organism/Length | Match Residues | for the Matched Portion | Value |
| P48052 | Carboxypeptidase A2 | 10 . . . 425 | 416/416 (100%) | 0.0 |
| | precursor (EC 3.4.17.15) - | 2 . . . 417 | 416/416 (100%) | |
| | Homo sapiens (Human), | | | |
| | 417 aa. | | | |
| A56171 | carboxypeptidase A2 (EC | 10 . . . 425 | 415/416 (99%) | 0.0 |
| | 3.4.17.15) precursor - | 2 . . . 417 | 415/416 (99%) | |
| | human, 417 aa. | | | |
| CAA02811 | SEQUENCE 3 FROM | 10 . . . 425 | 415/416 (99%) | 0.0 |
| | PATENT WO9513095 - | 2 . . . 417 | 415/416 (99%) | |
| | unidentified, 417 aa | | | |
| | (fragment). | | | |
| P19222 | Carboxypeptidase A2 | 10 . . . 425 | 362/416 (87%) | 0.0 |
| | precursor (EC 3.4.17.15) - | 2 . . . 417 | 384/416 (92%) | |
| | Rattus norvegicus (Rat), | | | |
| | 417 aa. | | | |
| Q9TV85 | Carboxypeptidase A1 (EC | 12 . . . 425 | 265/416 (63%) | e−158 |
| | 3.4.17.1) - Sus scrofa (Pig), | 4 . . . 419 | 325/416 (77%) | |
| | 419 aa. | | | |

PFam analysis predicts that the NOV27a protein contains the domains shown in the Table 27F.

TABLE 27F
Domain Analysis of NOV27a

| | NOV27a Match | Identities/Similarities | Expect |
|---|---|---|---|
| Pfam Domain | Region | for the Matched Region | Value |
| Propep_M14 | 32 . . . 109 | 41/82 (50%) | 1.6e−35 |
| | | 69/82 (84%) | |
| Zn_carbOpept | 129 . . . 408 | 161/304 (53%) | 4.8e−161 |
| | | 264/304 (87%) | |

### Example 28

The NOV28 clone was analyzed, and the nucleotide and encoded polypeptide sequences are shown in Table 28A.

TABLE 28A
NOV28 Sequence Analysis

| | |
|---|---|
| SEQ ID NO: 251 | 3032 bp |

| | |
|---|---|
| NOV28a, | CCACCATGGGCCACCATCACCACCATCACGAGGCCGCTCATGCTAAAACCACGGAGGAATGTTTGGC |
| CG57417-05 | |
| DNA Sequence | CTATTTTGGGGTGAGTGAGACCACGGGCCTCACCCCGGACCAAGTTAAGCGGAATCTGGAGAAATAC |
| | GGCCTCAATGAGCTCCCTGCTGAGGAAGCGAAGACCCTGTCGGAGCTGGTGATAGAGCAGTTTGAAG |
| | ACCTCCTGGTGCGGATTCTCCTCCTGGCCGCATGCATTTCCTTCGTGCTGGCCTGGTTTGAGGAAGG |
| | TGAAGAGACCATCACTGCCTTTGTTGAACCCTTTGTCATCCTCTTGATCCTCATTGCCAATGCCATC |
| | GTGGGGGTTTGGCAGGAGCGGAACGCAGAGAACGCCATCGAGGCCCTGAAGGAGTATGAGCCACAGA |
| | TGGGGAACGTCTACCGGGCTGACCGCAAGTCAGTGCAAAGGATCAAGGCTCCCGACATCGTCCCTGG |
| | GGACATCGTGGAGGTGGCTGTGGGGGACAAAGTCCCTGCAGACATCCGAATCCTCGCCATCAAATCC |
| | ACCACGCTGCGGGTTGACCAGTCCATCCTGACAGGCGAGTCTGTATCTGTCATCAAACACACGGAGC |
| | CCGTTCCTGACCCCCGAGCTGTCAACCAGGACAAGAAGAACATGCTTTTCTCGGGCACCAACATTGC |
| | AGCCGGCAAGGCCTTGGGCATCGTGGCCACCACTGGTGTGGGCACCGAGATTGGGAAGATCCGAGAC |
| | CAAATGGCTGCCACAGAACAGGACAAGACCCCCTTGCAGCAGAAGCTGGATGAGTTTGGGGAGCAGC |
| | TCTCCAAGGTCATCTCCCTCATCTGTGTGGCTGTCTGGCTTATCAACATTGGCCACTTCAACGACCC |
| | CGTCCATGGGGGCTCCTGGTTCCGCGGGGCCATCTACTACTTTAAGATTGCCGTGGCCTTGGCTGTG |
| | GCTGCCATCCCCGAAGGTCTTCCTGCAGTCATCACCACCTGCCTGGCCCTGGGTACCCGTCGGATGG |
| | CAAAGAAGAATGCCATTGTAAGAAGCTTGCCCTCCGTAGAGACCCTGGGCTGCACCTCTGTCATCTG |
| | TTCCGACAAGACAGGCACCCTCACCACCAACCAGATGTCTGTCTGCAAGATGTTTATCATTGACAAG |
| | GTGGATGGGGACATCTGCCTCCTGAATGAGTTCTCCATCACCGGCTCAACTTACGCTCCAGAGGGAG |
| | AGGTCTTGAAGAATGATAAGCCAGTCCGGCCAGGGCAGTATGACGGGCTGGTGGAGCTGGCCACCAT |
| | CTGTGCCCTCTGCAATGACTCCTCCTTGGACTTCAACGAGGCCAAAGGTGTCTATGAGAAGGTCGGC |
| | GAGGCCACCGAGACAGCACTCACCACCCTGGTGGAGAAGATGAATGTGTTCAACACGGATGTGAGAA |
| | GCCTCTCGAAGGTGGAGAGAGCCAACGCCTGCAACTCGGTGATCCGCCAGCTAATGAAGAAGGAATT |
| | CACCCTGGAGTTCTCCCGAGACAGAAAGTCCATGTCTGTCTATTGCTCCCCAGCCAAATCTTCCCGG |
| | GCTGCTGTGGGCAACAAGATGTTTGTCAAGGGTGCCCCTGAGGGCGTCATCGACCGCTGTAACTATG |
| | TGCGAGTTGGCACCACCCGGGTGCCACTGACGGGGCCGGTGAAGGAAAAGATCATGGCGGTGATCAA |
| | GGAGTGGGGCACTGGCCGGGACACCCTGCGCTGCTTGGCCCTGGCCACCCGGGACACCCCCCCGAAG |
| | CGAGAGGAAATGGTCCTGGATGACTCTGCCAGGTTCCTGGAGTATGAGACGGACCTGACATTCGTGG |
| | GTGTAGTGGGCATGCTGGACCCTCCGCGCAAGGAGGTCACGGGCTCCATCCAGCTGTGCCGTGACGC |
| | CGGGATCCGGGTGATCATGATCACTGGGGACAACAAGGGCACAGCCATTGCCATCTGCCGGCGAATT |
| | GGCATCTTTGGGGAGAACGAGGAGGTGGCCGATCGCGCCTACACGGGCCGAGAGTTCGACGACCTGC |
| | CCCTGGCTGAACAGCGGGAAGCCTGCCGACGTGCCTGCTGCTTCGCCCGTGTGGAGCCCTCGCACAA |
| | GTCCAAGATTGTGGAGTACCTGCAGTCCTACGATGAGATCACAGCCATGACAGGTGATGGCGTCAAT |
| | GACGCCCCTGCCCTGAAGAAGGCTGAGATTGGCATTGCCATGGGATCTGGCACTGCCGTGGCCAAGA |
| | CTGCCTCTGAGATGGTGCTGGCTGACGACAACTTCTCCACCATCGTAGCTGCTGTGGAGGAGGGCCG |
| | CGCCATCTACAACAACATGAAGCAGTTCATCCGCTACCTCATTTCCTCCAACGTGGGCGAGGTGGTC |
| | TGTATCTTCCTGACCGCTGCCCTGGGGCTGCCTGAGGCCCTGATCCCGGTGCAGCTGCTATGGGTGA |
| | ACTTGGTGACCGACGGGCTCCCAGCCACAGCCCTGGGCTTCAACCCACCAGACCTGGACATCATGGA |
| | CCGCCCCCCCCGGAGCCCCAAGGAGCCCCTCATCAGTGGCTGGCTCTTCTTCCGCTACATGGCAATC |
| | GGGGGCTATGTGGGTGCAGCCACCGTGGGAGCAGCTGCCTGGTGGTTCCTGTACGCTGAGGATGGGC |
| | CTCATGTCAACTACAGCCAGCTGACTCACTTCATGCAGTGCACCGAGGACAACACCCACTTTGAGGG |
| | CATAGACTGTGAGGTCTTCGAGGCCCCCGAGCCCATGACCATGGCCCTGTCCGTGCTGGTGACCATC |
| | GAGATGTGCAATGCACTGAACAGCCTGTCCGAGAACCAGTCCCTGCTGCGGATGCCACCCTGGGTGA |
| | ACATCTGGCTGCTGGGCTCCATCTGCCTCTCCATGTCCCTGCACTTCCTCATCCTCTATGTTGACCC |
| | CCTGCCGATGATCTTCAAGCTCCGGGCCCTGGACCTCACCCAGTGGCTCATGGTCCTCAAGATCTCA |
| | CTGCCAGTCATTGGGCTCGACGAAATCCTCAAGTTCGTTGCTCGGAACTACCTAGAGGATCCAGAAG |
| | ATGAAAGAAGGAAGTGA |

| | | |
|---|---|---|
| ORF Start: at 3 | | ORF Stop: TGA at 3030 |
| SEQ ID NO: 252 | 1009 aa | MW at 111232.1 kD |

| | |
|---|---|
| NOV28a, | TMGHHHHHHEAAHAKTTEECLAYFGVSETTGLTPDQVKRNLEKYGLNELPAEEGKTLWELVIEQFED |
| CG57417-05 | |
| Protein Sequence | LLVRILLLAACISFVLAWFEEGEETITAFVEPFVILLILIANAIVGVWQERNAENAIEALKEYEPEM |
| | GKVYRADRKSVQRIKARDIVPGDIVEVAVGDKVPADIRILAIKSTTLRVDQSILTGESVSVIKHTEP |
| | VPDPRAVNQDKKNMLFSGTNIAAGKALGIVATTGVGTEIGKIRDQMAATEQDKTPLQQKLDEFGEQL |
| | SKVISLICVAVWLINIGHFNDPVHGGSWFRGAIYYFKIAVALAVAAIPEGLPAVITTCLALGTRRMA |
| | KKNAIVRSLPSVETLGCTSVICSDKTGTLTTNQMSVCKMFIIDKVDGDICLLNEFSITGSTYAPEGE |
| | VLKNDKPVRPGQYDGLVELATICALCNDSSLDFNEAKGVYEKVGEATETALTTLVEKMNVFNTDVRS |
| | LSKVERANACNSVIRQLMKKEFTLEFSRDRKSMSVYCSPAKSSRAAVGNKMFVKGAPEGVIDRCNYV |
| | RVGTTRVPLTGPVKEKIMAVIKEWGTGRDTLRCLALATRDTPPKREEMVLDDSARFLEYETDLTFVG |
| | VVGMLDPPRKEVTGSIQLCRDAGIRVIMITGDNKGTAIAICRRIGIFGENEEVADRAYTGREFDDLP |
| | LAEQREACRRACCFARVEPSHKSKIVEYLQSYDEITAMTGDGVNDAPALKKAEIGIAMGSGTAVAKT |
| | ASEMVLADDNFSTIVAAVEEGRAIYNNMKQFIRYLISSNVGEVVCIFLTAALGLPEALIPVQLLWVN |
| | LVTDGLPATALGFNPPDLDIMDRPPRSPKEPLISGWLFFRYMAIGGYVGAATVGAAAWWFLYAEDGP |
| | HVNYSQLTHFMQCTEDNTHFEGIDCEVFEAPEPMTMALSVLVTIEMCNALNSLSENQSLLRMPPWVN |
| | IWLLGSICLSMSLHFLILYVDPLPMIFKLRALDLTQWLMVLKISLPVIGLDEILKFVARNYLEDPED |
| | ERRK |

| | |
|---|---|
| SEQ ID NO: 253 | 3040 bp |

| | |
|---|---|
| NOV28b, | ATGGAGGCCGCTCATGCTAAAACCACGGAGGAATGTTTGGCCTATTTTGGGGTGAGTGAGACCACGG |
| CG57417-03 | |
| DNA Sequence | GCCTCACCCCGGACCAAGTTAAGCGGAATCTGGAGAAATACGGCCTCAATGAGCTCCCTGCTGAGGA |
| | AGGGAAGACCCTGTGGGAGCTGGTGATAGAGCAGTTTGAAGACCTCCTGGTGCGGATTCTCCTCCTG |
| | GCCGCATGCATTTCCTTCGTGCTGGCCTGGTTTGAGGAAGGTGAAGAGACCATCACTGCCTTTGTTG |
| | AACCCTTTGTCATCCTCTTGATCCTCATTGCCAATGCCATCGTGGGGGTTTGGCAGGAGCGGAACGC |
| | AGAGAACGCCATCGAGGCCCTGAAGGAGTATGAGCCAGAGATGGGGAAGGTCTACCGGGCTGACCGC |
| | AAGTCAGTGCAAAGGATCAAGGCTCGGGACATCGTCCCTGGGGACATCGTGGAGGTGGCTGTGGGGG |
| | ACAAAGTCCCTGCAGACATCCGAATCCTCGCCATCAAATCCACCACGCTGCGGGTTGACCAGTCCAT |
| | CCTGACAGGCGAGTCTGTATCTGTCATCAAACACACGGAGCCCGTTCCTGACCCCCGAGCTGTCAAC |
| | CAGGACAAGAAGAACATGCTTTTCTCGGGCACCAACATTGCAGCCGGCAAGGCCTTGGGCATCGTGG |
| | CCACCACCGGTGTGGGCACCGAGATTGGGAAGATCCGAGACCAAATGGCTGCCACAGAACAGGACAA |
| | GACCCCCTTGCAGCAGAAGCTGGATGAGTTTGGGGAGCAGCTCTCCAAGGTCATCTCCCTCATCTGT |
| | GTGGCTGTCTGGCTTATCAACATTGGCCACTTCAACGACCCCGTCCATGGGGGCTCCTGGTTCCGCG |
| | GGGCCATCTACTACTTTAAGATTGCCGTGGCCTTGGCTGTGGCTGCCATCCCCGAAGGTCTTCCTGC |
| | AGTCATCACCACCTGCCTGGCCCTGGGTACCCGTCGGATGGCAAAGAAGAATGCCATTGTAAGAAGC |
| | TTGCCCTCCGTAGAGACCCTGGGCTGCACCTCTGTCATCTGTTCCGACAAGACAGGCACCCTCACCA |
| | CCAACCAGATGTCTGTCTGCAAGATGTTTATCATTGACAAGGTGGATGGGGACATCTGCCTCCTGAA |
| | TGAGTTCTCCATCACCGGCTCCACTTACGCTCCAGAGGGAGAGGTCTTGAAGAATGATAAGCCAGTC |
| | CGGCCAGGGCAGTATGACGGGCTGGTGGAGCTGGCCACCATCTGTGCCCTCTGCAATGACTCCTCCT |
| | TGGACTTCAACGAGGCCAAAGGTGTCTATGAGAAGGTCGGCGAGGCCACCGAGACAGCACTCACCAC |
| | CCTGGTGGAGAAGATGAATGTGTTCAACACGGATGTGAGAAGCCTCTCGAAGGTGGAGAGAGCCAAC |
| | GCCTGCAACTCGGTGATCCGCCAGCTAATGAAGAAGGAATTCACCCTGGAGTTCTCCCGAGACAGAA |
| | AGTCCATGTCTGTCTATTGCTCCCCAGCCAAATCTTCCCGGGCTGCTGTGGGCAACAAGATGTTTGT |
| | CAAGGGTGCCCCTGAGGGCGTCATCGACCGCTGTAACTATGTGCGAGTTGGCACCACCCGGGTGCCA |
| | CTGACGGGGCCGGTGAAGGAAAAGATCATGGCGGTGATCAAGGAGTGGGGCACTGGCCGGGACACCC |
| | TGCGCTGCTTGGCCCTGGCCACCCGGGACACCCCCCCGAAGCGAGAGGAAATGGTCCTGGATGACTC |
| | TGCCAGGTTCCTGGAGTATGAGACGGACCTGACATTCGTGGGTGTAGTGGGCATGCTGGACCCTCCG |
| | CGCAAGGAGGTCACGGGCTCCATCCAGCTGTGCCGTGACGCCGGGATCCGGGTGATCATGATCACTG |
| | GGGACAACAAGGGCACAGCCATTGCCATCTGCCGGCGAATTGGCATCTTTGGGGAGAACGAGGAGGT |
| | GGCCGATCGCGCCTACACGGGCCGAGAGTTCGACGACCTGCCCCTGGCTGAACAGCGGGAAGCCTGC |
| | CGACGTGCCTGCTGCTTCGCCCGTGTGGAGCCCTCGCACAAGTCCAAGATTGTGGAGTACCTGCAGT |
| | CCTACGATGAGATCACAGCCATGACAGGTGATGGCGTCAATGACGCCCCTGCCCTGAAGAAGGCTGA |
| | GATTGGCATTGCCATGGGATCTGGCACTGCCGTGGCCAAGACTGCCTCTGAGATGGTGCTGGCTGAC |
| | GACAACTTCTCCACCATCGTAGCTGCTGTGGAGGAGGGCCGCGCCATCTACAACAACATGAAGCAGT |
| | TCATCCGCTACCTCATTTCCTCCAACGTGGGCGAGGTGGTCTGTATCTTCCTGACCGCTGCCCTGGG |
| | GCTGCCTGAGGCCCTGATCCCGGTGCAGCTGCTATGGGTGAACTTGGTGACCGACGGGCTCCCAGCC |
| | ACAGCCCTGGGCTTCAACCCACCAGACCTGGACATCATGGACCGCCCCCCCCGGAGCCCCAAGGAGC |
| | CCCTCATCAGTGGCTGGCTCTTCTTCCGCTACATGGCAATCGGGGGCTATGTGGGTGCAGCCACCGT |
| | GGGAGCAGCTGCCTGGTGGTTCCTGTACGCTGAGGATGGGCCTCATGTCAACTACAGCCAGCTGACT |
| | CACTTCATGCAGTGCACCGAGGACAACACCCACTTTGAGGGCATAGACTGTGAGGTCTTCGAGGCCC |
| | CCGAGCCCATGACCATGGCCCTGTCCGTGCTGGTGACCATCGAGATGTGCAATGCACTGAACAGCCT |
| | GTCCGAGAACCAGTCCCTGCTGCGGATGCCACCCTGGGTGAACATCTGGCTGCTGGGCTCCATCTGC |
| | CTCTCCATGTCCCTGCACTTCCTCATCCTCTATGTTGACCCCCTGCCGATGATCTTCAAGCTCCGGG |
| | CCCTGGACCTCACCCAGTGGCTCATGGTCCTCAAGATCTCACTGCCAGTCATTGGGCTCGACGAAAT |
| | CCTCAAGTTCGTTGCTCGGAACTACCTAGAGGATCCAGAAGATGAAAGAAGGAAGTGAGCATCCTTT |
| | TGCTCTGTCCTCCCCACCCCGATAG |

| | | |
|---|---|---|
| ORF Start: ATG at 1 | | ORF Stop: TGA at 3004 |
| SEQ ID NO: 254 | 1001 aa | MW at 110251.1 kD |

| | |
|---|---|
| NOV28b, | MEAAHAKTTEECLAYFGVSETTGLTPDQVKRNLEKYGLNELPAEEGKTLWELVIEQFEDLLVRILLL |
| CG57417-03 | |
| Protein Sequence | AACISFVLAWFEEGEETITAFVEPFVILLILIANAIVGVWQERNAENAIEALKEYEPEMGKVYRADR |
| | KSVQRIKARDIVPGDIVEVAVGDKVPADIRILAIKSTTLRVDQSILTGESVSVIKHTEPVPDPRAVN |
| | QDKKNMLFSGTNIAAGKALGIVATTGVGTEIGKIRDQMAATEQDKTPLQQKLDEFGEQLSKVISLIC |
| | VAVWLINIGHFNDPVHGGSWFRGAIYYFKIAVALAVAAIPEGLPAVITTCLALGTRRMAKKNAIVRS |
| | LPSVETLGCTSVICSDKTGTLTTNQMSVCKMFIIDKVDGDICLLNEFSITGSTYAPEGEVLKNDKPV |
| | RPGQYDGLVELATICALCNDSSLDFNEAKGVYEKVGEATETALTTLVEKMNVFNTDVRSLSKVERAN |
| | ACNSVIRQLMKKEFTLEFSRDRKSMSVYCSPAKSSRAAVGNKMFVKGAPEGVIDRCNYVRVGTTRVP |
| | LTGPVKEKIMAVIKEWGTGRDTLRCLALATRDTPPKREEMVLDDSARFLEYETDLTFVGVVGMLDPP |
| | RKEVTGSIQLCRDAGIRVIMITGDNKGTAIAICRRIGIFGENEEVADRAYTGREFDDLPLAEQREAC |
| | RRACCFARVEPSHKSKIVEYLQSYDEITAMTGDGVNDAPALKKAEIGIAMGSGTAVAKTASEMVLAD |
| | DNFSTIVAAVEEGRAIYNNMKQFIRYLISSNVGEVVCIFLTAALGLPEALIPVQLLWVNLVTDGLPA |
| | TALGFNPPDLDIMDRPPRSPKEPLISGWLFFRYMAIGGYVGAATVGAAAWWFLYAEDGPHVNYSQLT |
| | HFMQCTEDNTHFEGIDCEVFEAPEPMTMALSVLVTIEMCNALNSLSENQSLLRMPPWVNIWLLGSIC |
| | LSMSLHFLILYVDPLPMIFKLRALDLTQWLMVLKISLPVIGLDEILKFVARNYLEDPEDERRK |

| | |
|---|---|
| SEQ ID NO: 255 | 3029 bp |

| | |
|---|---|
| NOV28c, | CCACCATGGAGGCCGCTCATGCTAAAACCACGGAGGAATGTTTGGCCTATTTTGGGGTGAGTGAGAC |
| 255169268 DNA | |
| Sequence | CACGGGCCTCACCCCGGACCAAGTTAAGCGGAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCGGGCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCGGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCAATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAAATCCTCAAGTTCGTTGCTCGGAACTACCTAGAGGATCCAGAAGATGAAAGAAGGAAGCACCATC |
| | ACCACCATCACTGA |

| | | |
|---|---|---|
| ORF Start: at 3 | | ORF Stop: TGA at 3027 |
| SEQ ID NO: 256 | 1008 aa | MW at 111175.1 kD |

| | |
|---|---|
| NOV28e, | TMEAAHAKTTEECLAYFGVSETTGLTPDQVKRNLEKYGLNELPAEEGKTLWELVTEQFEDLLVRILL |
| 255169268 | |
| Protein Sequence | LAACISFVLAWFEEGEETITAFVEPFVILLILIANAIVGVWQERNAENAIEALKEYEPEMGKVYRAD |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEDPEDERRKHHH |
| | HHH |

| | |
|---|---|
| SEQ ID NO: 257 | 3454 bp |

| | |
|---|---|
| NOV28d, | GAAAAAGAAGAAACCCAGGCAGACAGGCAGTTGGACACACTGAGGAAGACCCCCCACGAGTGGGAAC |
| CG57417-01 | |
| DNA Sequence | CCCCTGGAAGGAACACACCGGCCCCGGCCCCCAGGAAGGGAGCACAATGGAGGCCGCTCATGCTAAA |
| | ACCACGGAGGAATGTTTGGCCTATTTTGGGGTGAGTGAGACCACGGGCCTCACCCCGGACCAAGTTA |
| | AGCGGAATCTGGAGAAATACGGCCTCAATGAGCTCCCTGCTGAGGAAGGGAAGACCCTGTGGGAGCT |
| | GGTGATAGAGCAGTTTGAAGACCTCCTGGTGCGGATTCTCCTCCTGGCCGCATGCATTTCCTTCGTG |
| | CTGGCCTGGTTTGAGGAAGGTGAAGAGACCATCACTGCCTTTGTTGAACCCTTTGTCATCCTCTTGA |
| | TCCTCATTGCCAATGCCATCGTGGGGGTTTGGCAGGAGCGGAACGCAGAGAACGCCATCGAGGCCCT |
| | GAAGGAGTATGAGCCAGAGATGGGGAAGGTCTACCGGGCTGACCGCAAGTCAGTGCAAAGGATCAAG |
| | GCTCGGGACATCGTCCCTGGGGACATCGTGGAGGTGGCTGTGGGGGACAAAGTCCCTGCAGACATCC |
| | GAATCCTCGCCATCAAATCCACCACGCTGCGGGTTGACCAGTCCATCCTGACAGGCGAGTCTGTATC |
| | TGTCATCAAACACACGGAGCCCGTTCCTGACCCCCGAGCTGTCAACCAGGACAAGAAGAACATGCTT |
| | TTCTCGGGCACCAACATTGCAGCCGGCAAGGCCTTGGGCATCGTGGCCACCACTGGTGTGGGCACCG |
| | AGATTGGGAAGATCCGAGACCAAATGGCTGCCACAGAACAGGACAAGACCCCCTTGCAGCAGAAGCT |
| | GGATGAGTTTGGGGAGCAGCTCTCCAAGGTCATCTCCCTCATCTGTGTGGCTGTCTGGCTTATCAAC |
| | ATTGGCCACTTCAACGACCCCGTCCATGGGGGCTCCTGGTTCCGCGGGGCCATCTACTACTTTAAGA |
| | TTGCCGTGGCCTTGGCTGTGGCTGCCATCCCCGAAGGTCTTCCTGCAGTCATCACCACCTGCCTGGC |
| | CCTGGGTACCCGTCGGATGGCAAAGAAGAATGCCATTGTAAGAAGCTTGCCCTCCGTAGAGACCCTG |
| | GGCTGCACCTCTGTCATCTGTTCCGACAAGACAGGCACCCTCACCACCAACCAGATGTCTGTCTGCA |
| | AGATGTTTATCATTGACAAGGTGGATGGGGACATCTGCCTCCTGAATGAGTTCTCCATCACCGGCTC |
| | CACTTACGCTCCAGAGGGAGAGGTCTTGAAGAATGATAAGCCAGTCCGGCCAGGGCAGTATGACGGG |
| | CTGGTGGAGCTGGCCACCATCTGTGCCCTCTGCAATGACTCCTCCTTGGACTTCAACGAGGCCAAAG |
| | GTGTCTATGAGAAGGTCGGCGAGGCCACCGAGACAGCACTCACCACCCTGGTGGAGAAGATGAATGT |
| | GTTCAACACGGATGTGAGAAGCCTCTCGAAGGTGGAGAGAGCCAACGCCTGCAACTCGGTGATCCGC |
| | CAGCTAATGAAGAAGGAATTCACCCTGGAGTTCTCCCGAGACAGAAAGTCCATGTCTGTCTATTGCT |
| | CCCCAGCCAAATCTTCCCGGGCTGCTGTGGGCAACAAGATGTTTGTCAAGGGTGCCCCTGAGGGCGT |
| | CATCGACCGCTGTAACTATGTGCGAGTTGGCACCACCCGGGTGCCACTGACGGGGCCGGTGAAGGAA |
| | AAGATCATGGCGGTGATCAAGGAGTGGGGCACTGGCCGGGACACCCTGCGCTGCTTGGCCCTGGCCA |
| | CCCGGGACACCCCCCCGAAGCGAGAGGAAATGGTCCTGGATGACTCTGCCAGGTTCCTGGAGTATGA |
| | GACGGACCTGACATTCGTGGGTGTAGTGGGCATGCTGGACCCTCCGCGCAAGGAGGTCACGGGCTCC |
| | ATCCAGCTGTGCCGTGACGCCGGGATCCGGGTGATCATGATCACTGGGGACAACAAGGGCACAGCCA |
| | TTGCCATCTGCCGGCGAATTGGCATCTTTGGGGAGAACGAGGAGGTGGCCGATCGCGCCTACACGGG |
| | CCGAGAGTTCGACGACCTGCCCCTGGCTGAACAGCGGGAAGCCTGCCGACGTGCCTGCTGCTTCGCC |
| | CGTGTGGAGCCCTCGCACAAGTCCAAGATTGTGGAGTACCTGCAGTCCTACGATGAGATCACAGCCA |
| | TGACAGGTGATGGCGTCAATGACGCCCCTGCCCTGAAGAAGGCTGAGATTGGCATTGCCATGGGATC |
| | TGGCACTGCCGTGGCCAAGACTGCCTCTGAGATGGTGCTGGCTGACGACAACTTCTCCACCATCGTA |
| | GCTGCTGTGGAGGAGGGCCGCGCCATCTACAACAACATGAAGCAGTTCATCCGCTACCTCATTTCCT |
| | CCAACGTGGGCGAGGTGGTCTGTATCTTCCTGACCGCTGCCCTGGGGCTGCCTGAGGCCCTGATCCC |
| | GGTGCAGCTGCTATGGGTGAACTTGGTGACCGACGGGCTCCCAGCCACAGCCCTGGGCTTCAACCCA |
| | CCAGACCTGGACATCATGGACCGCCCCCCCCGGAGCCCCAAGGAGCCCCTCATCAGTGGCTGGCTCT |
| | TCTTCCGCTACATGGCAATCGGGGGCTATGTGGGTGCAGCCACCGTGGGAGCAGCTGCCTGGTGGTT |
| | CCTGTACGCTGAGGATGGGCCTCATGTCAACTACAGCCAGCTGACTCACTTCATGCAGTGCACTGAG |
| | CATAACCCTGAATTTGATGGCCTGGACTGCGAGGTCTTTGAAGCCCCCGAGCCCATGACCATGGCCT |
| | TGTCTGTGTTGGTGACCATCGAGATGTGCAACGCCCTCAACAGCCTGTCTGAGAACCAGTCCCTACT |
| | GCGGATGCCGCCCTGGGTGAACATCTGGCTTCTCGGTTCCATCTGCCTGTCCATGTCCCTCCACTTC |
| | CTCATCCTCTATGTTGACCCCCTGCCGATGATCTTCAAGCTCCGGGCCCTGGACCTCACCCAGTGGC |
| | TCATGGTCCTCAAGATCTCACTGCCAGTCATTGGGCTCGACGAAATCCTCAAGTTCGTTGCTCGGAA |
| | CTACCTAGAGGGATAACTGTTCCCCCTCCTCCATCTCTGAGCCCGTGTCACAGATCCAGAAGATGAA |
| | AGAAGGAAGTGAGCATCCTTTTGCTCTGTCCTCCCCACCCCGATAGTGACACATCTTCAGGCAGAGC |
| | TGTGGCACAGACCCCCGTCCTGTCCCCCACACCCGTGTCATGTGTCTGTTTATAAACATGTCCCCTT |
| | CCCTTTCCTTCCCCCTCGGCCACCCGCCTCCCTCTCAACCTTGTAAATTCCCCTTCCCAACCCCGAG |
| | GGGCTTGCAGGGACAAGGCGACCGACTGCGCTGAGCTGCTTATTTATTGAAAATAAACGACGGAAAA |
| | GTCAAAAAAAAAAAATAAAAAAAAAAAAAAAAAAAAA |

| | | |
|---|---|---|
| ORF Start: ATG at 114 | | ORF Stop: TAA at 3096 |
| SEQ ID NO: 258 | 994 aa | MW at 109278.1 kD |

| | |
|---|---|
| NOV28d, | MEAAHAKTTEECLAYFGVSETTGLTPDQVKRNLEKYGLNELPAEEGKTLWELVIEQFEDLLVRILLL |
| CG57417-01 | |
| Protein Sequence | AACISFVLAWFEEGEETITAFVEPFVILLILIANAIVGVWQERNAENAIEALKEYEPEMGKVYRADR |
| | KSVQRIKARDIVPGDIVEVAVGDKVPADIRILAIKSTTLRVDQSILTGESVSVIKHTEPVPDPRAVN |
| | QDKKNMLFSGTNIAAGKALGIVATTGVGTEIGKIRDQMAATEQDKTPLQQKLDEFGEQLSKVISLIC |
| | VAVWLINIGHFNDPVHGGSWFRGAIYYFKIAVALAVAAIPEGLPAVITTCLALGTRRMAKKNAIVRS |
| | LPSVETLGCTSVICSDKTGTLTTNQMSVCKMFIIDKVDGDICLLNEFSITGSTYAPEGEVLKNDKPV |
| | RPGQYDGLVELATICALCNDSSLDFNEAKGVYEKVGEATETALTTLVEKMNVFNTDVRSLSKVERAN |
| | ACNSVIRQLMKKEFTLEFSRDRKSMSVYCSPAKSSRAAVGNKMFVKGAPEGVIDRCNYVRVGTTRVP |
| | LTGPVKEKIMAVIKEWGTGRDTLRCLALATRDTPPKREEMVLDDSARFLEYETDLTFVGVVGMLDPP |
| | RKEVTGSIQLCRDAGIRVIMITGDNKGTAIAICRRIGIFGENEEVADRAYTGREFDDLPLAEQREAC |
| | RRACCFARVEPSHKSKIVEYLQSYDEITAMTGDGVNDAPALKKAEIGIAMGSGTAVAKTASEMVLAD |
| | DNFSTIVAAVEEGRAIYNNMKQFIRYLISSNVGEVVCIFLTAALGLPEALIPVQLLWVNLVTDGLPA |
| | TALGFNPPDLDIMDRPPRSPKEPLISGWLFFRYMAIGGYVGAATVGAAAWWFLYAEDGPHVNYSQLT |
| | HFMQCTEDNTHFEGIDCEVFEAPEPMTMALSVLVTIEMCNALNSLSENQSLLRMPPWVNIWLLGSIC |
| | LSMSLHFLILYVDPLPMIFKLRALDLTQWLMVLKISLPVIGLDEILKFVARNYLEG |

| | |
|---|---|
| SEQ ID NO: 259 | 2999 bp |

| | |
|---|---|
| NOV28e, | CCACCATGGAGGCCGCTCATGCTAAAACCACGGAGGAATGTTTGGCCTATTTTGGGGTGAGTGAGAC |
| 181356924 DNA | |
| Sequence | CACGGGCCTCACCCCGGACCAAGTTAAGCGGAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCGGGCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCGGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCCATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAATCCTCAAGTTCGTTGCTCGGAACTACCTAGAGGGATAATCTAGAGGG |

| | | |
|---|---|---|
| ORF Start: at 3 | | ORF Stop: TAA at 2988 |
| SEQ ID NO: 260 | 995 aa | MW at 109383.2 kD |

| | |
|---|---|
| NOV28e, | TMEAAHAKTTEECLAYFGVSETTGLTPDQVKRNLEKYGLNELPAEEGKTLWELVIEQFEDLLVRILL |
| 181356924 | |
| Protein Sequence | LAACISFVLAWFEEGEETITAFVEPFVILLILIANAIVGVWQERNAENAIEALKEYEPEMGKVYRAD |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEG |

| | |
|---|---|
| SEQ ID NO: 261 | 3029 bp |

| | |
|---|---|
| NOV28f, | CCACCATGGAGGCCGCTCATGCTAAAACCACGGAGGAATGTTTGGCCTATTTTGGGGTGAGTGAGAC |
| 255169268 | |
| DNA Sequence | CACGGGCCTCACCCCGGACCAAGTTAAGCGGAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCGGGCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCGGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCCATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAAATCCTCAAGTTCGTTGCTCGGAACTACCTAGAGGATCCAGAAGATGAAAGAAGGAAGCACCATC |
| | ACCACCATCACTGA |

| | | |
|---|---|---|
| ORF Start: at 3 | | ORF Stop: TGA at 3027 |
| SEQ ID NO: 262 | 1008 aa | MW at 111175.1 kD |

| | |
|---|---|
| NOV28f, | TMEAAHAKTTEECLAYFGVSETTGLTPDQVKRNLEKYGLNELPAEEGKTLWELVIEQFEDLLVRILL |
| 255169268 | |
| Protein | LAACISFVLAWFEEGEETITAFVEPFVILLILIANAIVGVWQERNAENAIEALKEYEPEMGKVYRAD |
| Sequence | |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEDPEDERRKHHH |
| | HHH |

| | |
|---|---|
| SEQ ID NO: 263 | 3010 bp |

| | |
|---|---|
| NOV28g, | CCACCATGGAGGCCGCTCATGCTAAAACCACGGAGGAATGTTTGGCCTATTTTGGGGTGAGTGAGAC |
| 206977032 DNA | |
| Sequence | CACGGGCCTCACCCCGGACCAAGTTAAGCTTAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCGGGCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCGGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCCATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAAATCCTCAAGTTCGTTGCTCGGAACTACCTAGAGGATCCAGAAGATGAAAGAAGGAAGTG |

| | | |
|---|---|---|
| ORF Start: at 3 | | ORF Stop: end of sequence |
| SEQ ID NO: 264 | 1003 aa | MW at 110352.2 kD |

| | |
|---|---|
| NOV28g, | TMEAAHAKTTEECLAYFGVSETTGLTPDQVKRNLEKYGLNELPAEEGKTLWELVIEQFEDLLVRILL |
| 206977032 | |
| Protein Sequence | LAACISFVLAWFEEGEETITAFVEPFVILLILIANAIVGVWQERNAENAIEALKEYEPEMGKVYRAD |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEDPEDERRKX |

| | |
|---|---|
| SEQ ID NO: 265 | 3011 bp |

| | |
|---|---|
| NOV28h, | CCACCATGGAGGCCGCTCATGCTAAAACCACGGAGGAATGTTTGGCCTATTTTGGGGTGAGTGAGAC |
| 201190923 DNA | |
| Sequence | CACGGGCCTCACCCCGGACCAAGTTAAGCGGAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCGGGCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCGGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCCATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAAATCCTCAAGTTCGTTGCTCGGAACTACCTAGAGGATCCAGAAGATGAAAGAAGGAAGTGA |

| | | |
|---|---|---|
| ORF Start: at 3 | | ORF Stop: TGA at 3009 |
| SEQ ID NO: 266 | 1002 aa | MW at 110352.2 kD |

| | |
|---|---|
| NOV28h, | TMEAAHAKTTEECLAYFGVSETTGLTPDQVKRNLEKYGLNELPAEEGKTLWELVIEQFEDLLVRILL |
| 201190923 | |
| Protein Sequence | LAACISFVLAWFEEGEETITAFVEPFVILLILIANAIVGVWQERNAENAIEALKEYEPEMGKVYRAD |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEDPEDERRK |

| | |
|---|---|
| SEQ ID NO: 267 | 3040 bp |

| | |
|---|---|
| NOV28i, | ATGGAGGCCGCTCATGCTAAAACCACGGAGGAATGTTTGGCCTATTTTGGGGTGAGTGAGACCACGG |
| CG57417-02 | |
| DNA Sequence | GCCTCACCCCGGACCAAGTTAAGCGGAATCTGGAGAAATACGGCCTCAATGAGCTCCCTGCTGAGGA |
| | AGGGAAGACCCTGTGGGAGCTGGTGATAGAGCAGTTTGAAGACCTCCTGGTGCGGATTCTCCTCCTG |
| | GCCGCATGCATTTCCTTCGTGCTGGCCTGGTTTGAGGAAGGTGAAGAGACCATCACTGCCTTTGTTG |
| | AACCCTTTGTCATCCTCTTGATCCTCATTGCCAATGCCATCGTGGGGGTTTGGCAGGAGCGGAACGC |
| | AGAGAACGCCATCGAGGCCCTGAAGGAGTATGAGCCAGAGATGGGGAAGGTCTACCGGGCTGACCGC |
| | AAGTCAGTGCAAAGGATCAAGGCTCGGGACATCGTCCCTGGGGACATCGTGGAGGTGGCTGTGGGGG |
| | ACAAAGTCCCTGCAGACATCCGAATCCTCGCCATCAAATCCACCACGCTGCGGGTTGACCAGTCCAT |
| | CCTGACAGGCGAGTCTGTATCTGTCATCAAACACACGGAGCCCGTTCCTGACCCCCGAGCTGTCAAC |
| | CAGGACAAGAAGAACATGCTTTTCTCGGGCACCAACATTGCAGCCGGCAAGGCCTTGGGCATCGTGG |
| | CCACCACCGGTGTGGGCACCGAGATTGGGAAGATCCGAGACCAAATGGCTGCCACAGAACAGGACAA |
| | GACCCCCTGGCAGCAGAAGCTGGATGAGTTTGGGGAGCAGCTCTCCAAGGTCATCTCCCTCATCTGT |
| | GTGGCTGTCTGGCTTATCAACATTGGCCACTTCAACGACCCCGTCCATGGGGGCTCCTGGTTCCGCG |
| | GGGCCATCTACTACTTTAAGATTGCCGTGGCCTTGGCTGTGGCTGCCATCCCCGAAGGTCTTCCTGC |
| | AGTCATCACCACCTGCCTGGCCCTGGGTACCCGTCGGATGGCAAAGAAGAATGCCATTGTAAGAAGC |
| | TTGCCCTCCGTAGAGACCCTGGGCTGCACCTCTGTCATCTGTTCCGACAAGACAGGCACCCTCACCA |
| | CCAACCAGATGTCTGTCTGCAAGATGTTTATCATTGACAAGGTGGATGGGGACATCTGCCTCCTGAA |
| | TGAGTTCTCCATCACCGGCTCCACTTACGCTCCAGAGGGAGAGGTCTTGAAGAATGATAAGCCAGTC |
| | CGGCCAGGGCAGTATGACGGGCTGGTGGAGCTGGCCACCATCTGTGCCCTCTGCAATGACTCCTCCT |
| | TGGACTTCAACGAGGCCAAAGGTGTCTATGAGAAGGTCGGCGAGGCCACCGAGACAGCACTCACCAC |
| | CCTGGTGGAGAAGATGAATGTGTTCAACACGGATGTGAGAAGCCTCTCGAAGGTGGAGAGAGCCAAC |
| | GCCTGCAACTCGGTGATCCGCCAGCTAATGAAGAAGGAATTCACCCTGGAGTTCTCCCGAGACAGAA |
| | AGTCCATGTCTGTCTATTGCTCCCCAGCCAAATCTTCCCGGGCTGCTGTGGGCAACAAGATGTTTGT |
| | CAAGGGTGCCCCTGAGGGCGTCATCGACCGCTGTAACTATGTGCGAGTTGGCACCACCCGGGTGCCA |
| | CTGACGGGGCCGGTGAAGGAAAAGATCATGGCGGTGATCAAGGAGTGGGGCACTGGCCGGGACACCC |
| | TGCGCTGCTTGGCCCTGGCCACCCGGGACACCCCCCCGAAGCGAGAGGAAATGGTCCTGGATGACTC |
| | TGCCAGGTTCCTGGAGTATGAGACGGACCTGACATTCGTGGGTGTAGTGGGCATGCTGGACCCTCCG |
| | CGCAAGGAGGTCACGGGCTCCATCCAGCTGTGCCGTGACGCCGGGATCCGGGTGATCATGATCACTG |
| | GGGACAACAAGGGCACAGCCATTGCCATCTGCCGGCGAATTGGCATCTTTGGGGAGAACGAGGAGGT |
| | GGCCGATCGCGCCTACACGGGCCGAGAGTTCGACGACCTGCCCCTGGCTGAACAGCGGGAAGCCTGC |
| | CGACGTGCCTGCTGCTTCGCCCGTGTGGAGCCCTCGCACAAGTCCAAGATTGTGGAGTACCTGCAGT |
| | CCTACGATGAGATCACAGCCATGACAGGTGATGGCGTCAATGACGCCCCTGCCCTGAAGAAGGCTGA |
| | GATTGGCATTGCCATGGGATCTGGCACTGCCGTGGCCAAGACTGCCTCTGAGATGGTGCTGGCTGAC |
| | GACAACTTCTCCACCATCGTAGCTGCTGTGGAGGAGGGCCGCGCCATCTACAACAACATGAAGCAGT |
| | TCATCCGCTACCTCATTTCCTCCAACGTGGGCGAGGTGGTCTGTATCTTCCTGACCGCTGCCCTGGG |
| | GCTGCCTGAGGCCCTGATCCCGGTGCAGCTGCTATGGGTGAACTTGGTGACCGACGGGCTCCCAGCC |
| | ACAGCCCTGGGCTTCAACCCACCAGACCTGGACATCATGGACCGCCCCCCCCGGAGCCCCAAGGAGC |
| | CCCTCATCAGTGGCTGGCTCTTCTTCCGCTACATGGCAATCGGGGGCTATGTGGGTGCAGCCACCGT |
| | GGGAGCAGCTGCCTGGTGGTTCCTGTACGCTGAGGATGGGCCTCATGTCAACTACAGCCAGCTGACT |
| | CACTTCATGCAGTGCACCGAGGACAACACCCACTTTGAGGGCATAGACTGTGAGGTCTTCGAGGCCC |
| | CCGAGCCCATGACCATGGCCCTGTCCGTGCTGGTGACCATCGAGATGTGCAATGCACTGAACAGCCT |
| | GTCCGAGAACCAGTCCCTGCTGCGGATGCCACCCTGGGTGAACATCTGGCTGCTGGGCTCCATCTGC |
| | CTCTCCATGTCCCTGCACTTCCTCATCCTCTATGTTGACCCCCTGCCGATGATCTTCAAGCTCCGGG |
| | CCCTGGACCTCACCCAGTGGCTCATGGTCCTCAAGATCTCACTGCCAGTCATTGGGCTCGACGAAAT |
| | CCTCAAGTTCGTTGCTCGGAACTACCTAGAGGATCCAGAAGATGAAAGAAGGAAGTGAGCATCCTTT |
| | TGCTCTGTCCTCCCCACCCCGATAG |

| | | |
|---|---|---|
| ORF Start: ATG at 1 | | ORF Stop: TGA at 3004 |
| SEQ ID NO: 268 | 1001 aa | MW at 110251.1 kD |

| | |
|---|---|
| NOV28i, | MEAAHAKTTEECLAYFGVSETTGLTPDQVKRNLEKYGLNELPAEEGKTLWELVIEQFEDLLVRILLL |
| CG57417-02 | |
| Protein | AACISFVLAWFEEGEETITAFVEPFVILLILIANAIVGVWQERNAENAIEALKEYEPEMGKVYRADR |
| Sequence | |
| | KSVQRIKARDIVPGDIVEVAVGDKVPADIRILAIKSTTLRVDQSILTGESVSVIKHTEPVPDPRAVN |
| | QDKKNMLFSGTNIAAGKALGIVATTGVGTEIGKIRDQMAATEQDKTPLQQKLDEFGEQLSKVISLIC |
| | VAVWLINIGHFNDPVHGGSWFRGAIYYFKIAVALAVAAIPEGLPAVITTCLALGTRRMAKKNAIVRS |
| | LPSVETLGCTSVICSDKTGTLTTNQMSVCKMFIIDKVDGDICLLNEFSITGSTYAPEGEVLKNDKPV |
| | RPGQYDGLVELATICALCNDSSLDFNEAKGVYEKVGEATETALTTLVEKMNVFNTDVRSLSKVERAN |
| | ACNSVIRQLMKKEFTLEFSRDRKSMSVYCSPAKSSRAAVGNKMFVKGAPEGVIDRCNYVRVGTTRVP |
| | LTGPVKEKIMAVIKEWGTGRDTLRCLALATRDTPPKREEMVLDDSARFLEYETDLTFVGVVGMLDPP |
| | RKEVTGSIQLCRDAGIRVIMITGDNKGTAIAICRRIGIFGENEEVADRAYTGREFDDLPLAEQREAC |
| | RRACCFARVEPSHKSKIVEYLQSYDEITAMTGDGVNDAPALKKAEIGIAMGSGTAVAKTASEMVLAD |
| | DNFSTIVAAVEEGRAIYNNMKQFIRYLISSNVGEVVCIFLTAALGLPEALIPVQLLWVNLVTDGLPA |
| | TALGFNPPDLDIMDRPPRSPKEPLISGWLFFRYMAIGGYVGAATVGAAAWWFLYAEDGPHVNYSQLT |
| | HFMQCTEDNTHFEGIDCEVFEAPEPMTMALSVLVTIEMCNALNSLSENQSLLRMPPWVNIWLLGSIC |
| | LSMSLHFLILYVDPLPMIFKLRALDLTQWLMVLKISLPVIGLDEILKFVARNYLEDPEDERRK |

| | |
|---|---|
| SEQ ID NO: 269 | 3010 bp |

| | |
|---|---|
| NOV28j, | CCACCATGGAGGCCGCTCATGCTAAAACCACGGAGGAATGTTTGGCCTATTTTGGGGTGAGTGAGAC |
| CG57417-04 | |
| DNA Sequence | CACGGGCCTCACCCCGGACCAAGTTAAGCGGAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCGGGCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCGGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCCATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAAATCCTCAAGTTCGTTGCTCGGAACTACCTAGAGGATCCAGAAGATGAAAGAAGGAAGTG |

| | | |
|---|---|---|
| ORF Start: at 3 | | ORF Stop: at 3009 |
| SEQ ID NO: 270 | 1002aa | MW at 110352.2 kD |

| | |
|---|---|
| NOV28j, | TMEAAHAKTTEECLAYFGVSETTGLTPDQVKRNLEKYGLNELPAEEGKTLWELVIEQFEDLLVRILL |
| CG57417-04 | |
| Protein | LAACISFVLAWFEEGEETITAFVEPFVILLILIANAIVGVWQERNAENAIEALKEYEPEMGKVYRAD |
| Sequence | |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEDPEDERRK |

| | |
|---|---|
| SEQ ID NO: 271 | 3029 bp |

| | |
|---|---|
| NOV28k, | CCACCATGGAGGCCGCTCATGCTAAAACCACGGAGGAATGTTTGGCCTATTTTGGGGTGAGTGAGAC |
| CG57417-06 | |
| DNA Sequence | CACGGGCCTCACCCCGGACCAAGTTAAGCGGAATCTGGAGAAATACGGCCTCAATGAGCTCCCTGCT |
| | GAGGAAGGGAAGACCCTGTGGGAGCTGGTGATAGAGCAGTTTGAAGACCTCCTGGTGCGGATTCTCC |
| | TCCTGGCCGCATGCATTTCCTTCGTGCTGGCCTGGTTTGAGGAAGGTGAAGAGACCATCACTGCCTT |
| | TGTTGAACCCTTTGTCATCCTCTTGATCCTCATTGCCAATGCCATCGTGGGGGTTTGGCAGGAGCGG |
| | AACGCAGAGAACGCCATCGAGGCCCTGAAGGAGTATGAGCCAGAGATGGGGAAGGTCTACCGGGCTG |
| | ACCGCAAGTCAGTGCAAAGGATCAAGGCTCGGGACATCGTCCCTGGGGACATCGTGGAGGTGGCTGT |
| | GGGGGACAAAGTCCCTGCAGACATCCGAATCCTCGCCATCAAATCCACCACGCTGCGGGTTGACCAG |
| | TCCATCCTGACAGGCGAGTCTGTATCTGTCATCAAACACACGGAGCCCGTTCCTGACCCCCGAGCTG |
| | TCAACCAGGACAAGAAGAACATGCTTTTCTCTTTCACCAACATTGCAGCCGGCAAGGCCTTGGGCAT |
| | CGTGGCCACCACTGGTGTGGGCACCGAGATTGGGAAGATCCGAGACCAAATGGCTGCCACAGAACAG |
| | GACAAGACCCCCTTGCAGCAGAAGCTGGATGAGTTTGGGGAGCAGCTCTCCAAGGTCATCTCCCTCA |
| | TCTGTGTGGCTGTCTGGCTTATCAACATTGGCCACTTCAACGACCCCGTCCATGGGGGCTCCTGGTT |
| | CCGCGGGGCCATCTACTACTTTAAGATTGCCGTGGCCTTGGCTGTGGCTGCCATCCCCGAAGGTCTT |
| | CCTGCAGTCATCACCACCTGCCTGGCCCTGGGTACCCGTCGGATGGCAAAGAAGAATGCCATTGTAA |
| | GAAGCTTGCCCTCCGTAGAGACCCTGGGCTGCACCTCTGTCATCTGTTCCGACAAGACAGGCACCCT |
| | CACCACCAACCAGATGTCTGTCTGCAAGATGTTTATCATTGACAAGGTGGATGGGGACATCTGCCTC |
| | CTGAATGAGTTCTCCATCACCGGCTCCACTTACGCTCCAGAGGGAGAGGTCTTGAAGAATGATAAGC |
| | CAGTCCGGCCAGGGCAGTATGACGGGCTGGTGGAGCTGGCCACCATCTGTGCCCTCTGCAATGACTC |
| | CTCCTTGGACTTCAACGAGGCCAAAGGTGTCTATGAGAAGGTCGGCGAGGCCACCGAGACAGCACTC |
| | ACCACCCTGGTGGAGAAGATGAATGTGTTCAACACGGATGTGAGAAGCCTCTCGAAGGTGGAGAGAG |
| | CCAACGCCTGCAACTCGGTGATCCGCCAGCTAATGAAGAAGGAATTCACCCTGGAGTTCTCCCGAGA |
| | CAGAAAGTCCATGTCTGTCTATTGCTCCCCAGCCAAATCTTCCCGGGCTGCTGTGGGCAACAAGATG |
| | TTTGTCAAGGGTGCCCCTGAGGGCGTCATCGACCGCTGTAACTATGTGCGAGTTGGCACCACCCGGG |
| | TGCCACTGACGGGGCCGGTGAAGGAAAAGATCATGGCGGTGATCAAGGAGTGGGGCACTGGCCGGGA |
| | CACCCTGCGCTGCTTGGCCCTGGCCACCCCGGACACCCCCCCGAAGCGAGAGGAAATGGTCCTGGAT |
| | GACTCTGCCAGGTTCCTGGAGTATGAGACGGACCTGACATTCGTGGGTGTAGTGGGCATGCTGGACC |
| | CTCCGCGCAAGGAGGTCACGGGCTCCATCCAGCTGTGCCGTGACGCCGGGATCCGGGTGATCATGAT |
| | CACTGGGGACAACAAGGGCACAGCCATTGCCATCTGCCGGCGAATTGGCATCTTTGGGGAGAACGAG |
| | GAGGTGGCCGATCGCGCCTACACGGGCCGAGAGTTCGACGACCTGCCCCTGGCTGAACAGCGGGAAG |
| | CCTGCCGACGTGCCTGCTGCTTCGCCCGTGTGGAGCCCTCGCACAAGTCCAAGATTGTGGAGTACCT |
| | GCAGTCCTACGATGAGATCACAGCCATGACAGGTGATGGCGTCAATGACGCCCCTGCCCTGAAGAAG |
| | GCTGAGATTGGCATTGCCATGGGATCTGGCACTGCCGTGGCCAAGACTGCCTCTGAGATGGTGCTGG |
| | CTGACGACAACTTCTCCACCATCGTAGCTGCTGTGGAGGAGGGCCGCGCCATCTACAACAACATGAA |
| | GCAGTTCATCCGCTACCTCATTTCCTCCAACGTGGGCGAGGTGGTCTGTATCTTCCTGACCGCTGCC |
| | CTGGGGCTGCCTGAGGCCCTGATCCCGGTGCAGCTGCTATGGGTGAACTTGGTGACCGACGGGCTCC |
| | CAGCCACAGCCCTGGGCTTCAACCCACCAGACCTGGACATCATGGACCGCCCCCCCCGGAGCCCCAA |
| | GGAGCCCCTCATCAGTGGCTGGCTCTTCTTCCGCTACATGGCAATCGGGGGCTATGTGGGTGCAGCC |
| | ACCGTGGGAGCAGCTGCCTGGTGGTTCCTGTACGCTGAGGATGGGCCTCATGTCAACTACAGCCAGC |
| | TGACTCACTTCATGCAGTGCACCGAGGACAACACCCACTTTGAGGGCATAGACTGTGAGGTCTTCGA |
| | GGCCCCCGAGCCCATGACCATGGCCCTGTCCGTGCTGGTGACCATCGAGATGTGCAATGCACTGAAC |
| | AGCCTGTCCGAGAACCAGTCCCTGCTGCGGATGCCACCCTGGGTGAACATCTGGCTGCTGGGCTCCA |
| | TCTGCCTCTCCATGTCCCTGCACTTCCTCATCCTCTATGTTGACCCCCTGCCGATGATCTTCAAGCT |
| | CCGGGCCCTGGACCTCACCCAGTGGCTCATGGTCCTCAAGATCTCACTGCCAGTCATTGGGCTCGAC |
| | GAAATCCTCAAGTTCGTTGCTTGGAACTACCTAGAGGATCCAGAAGATGAAAGAAGGAAGCACCATC |
| | ACCACCATCACTGA |

| | | |
|---|---|---|
| ORF Start: at 3 | | ORF Stop: TGA at 3027 |
| SEQ ID NO: 272 | 1008 aa | MW at 111175.1 kD |

| | |
|---|---|
| NOV28k, | TMEAAHAKTTEECLAYFGVSETTGLTPDQVKRNLEKYGLNELPAEEGKTLWELVIEQFEDLLVRILL |
| CG57417-06 | |
| Protein Sequence | LAACISFVLAWFEEGEETITAFVEPFVILLILIANAIVGVWQERNAENAIEALKEYEPEMGKVYRAD |
| | RKSVQRIKARDIVPGDIVEVAVGDKVPADIRILAIKSTTLRVDQSILTGESVSVIKHTEPVPDPRAV |
| | NQDKKNMLFSGTNIAAGKALGIVATTGVGTEIGKIRDQMAATEQDKTPLQQKLDEFGEQLSKVISLI |
| | CVAVWLINIGHFNDPVHGGSWFRGAIYYFKIAVALAVAAIPEGLPAVITTCLALGTRRMAKKNAIVR |
| | SLPSVETLGCTSVICSDKTGTLTTNQMSVCKMFIIDKVDGDICLLNEFSITGSTYAPEGEVLKNDKP |
| | VRPGQYDGLVELATICALCNDSSLDFNEAKGVYEKVGEATETALTTLVEKMNVFNTDVRSLSKVERA |
| | NACNSVIRQLMKKEFTLEFSRDRKSMSVYCSPAKSSRAAVGNKMFVKGAPEGVIDRCNYVRVGTTRV |
| | PLTGPVKEKIMAVIKEWGTGRDTLRCLALATRDTPPKREEMVLDDSARFLEYETDLTFVGVVGMLDP |
| | PRKEVTGSIQLCRDAGIRVIMITGDNKGTAIAICRRIGIFGENEEVADRAYTGREFDDLPLAEQREA |
| | CRRACCFARVEPSHKSKIVEYLQSYDEITAMTGDGVNDAPALKKAEIGIAMGSGTAVAKTASEMVLA |
| | DDNFSTIVAAVEEGRAIYNNMKQFIRYLISSNVGEVVCIFLTAALGLPEALIPVQLLWVNLVTDGLP |
| | ATALGFNPPDLDIMDRPPRSPKEPLISGWLFFRYMAIGGYVGAATVGAAAWWFLYAEDGPHVNYSQL |
| | THFMQCTEDNTHFEGIDCEVFEAPEPMTMALSVLVTIEMCNALNSLSENQSLLRMPPWVNIWLLGSI |
| | CLSMSLHFLILYVDPLPMIFKLRALDLTQWLMVLKISLPVIGLDEILKFVARNYLEDPEDERRKHHH |
| | HHH |

| | |
|---|---|
| SEQ ID NO: 273 | 3011 bp |

| | |
|---|---|
| N0V28l, | CCACCATGGGCCACCATCACCACCATCACGAGGCCGCTCATGCTAAAACCACGGAGGAATGTTTGGC |
| CG57417-07 | |
| DNA Sequence | CTATTTTGGGGTGAGTGAGACCACGGGCCTCACCCCGGACCAAGTTAAGCGGAATCTGGAGAAATAC |
| | GGCCTCAATGAGCTCCCTGCTGAGGAAGGGAAGACCCTGTGGGAGCTGGTGATAGAGCAGTTTGAAG |
| | ACCTCCTGGTGCGGATTCTCCTCCTGGCCGCATGCATTTCCTTCGTGCTGGCCTGGTTTGAGGAAGG |
| | TGAAGAGACCATCACTGCCTTTGTTGAACCCTTTGTCATCCTCTTGATCCTCATTGCCAATGCCATC |
| | GTGGGGGTTTGGCAGGAGCGGAACGCAGAGAACGCCATCGAGGCCCTGAAGGAGTATGAGCCAGAGA |
| | TGGGGAAGGTCTACCGGGCTGACCGCAAGTCAGTGCAAAGGATCAAGGCTCGGGACATCGTCCCTGG |
| | GGACATCGTGGAGGTGGCTGTGGGGGACAAAGTCCCTGCAGACATCCGAATCCTCGCCATCAAATCC |
| | ACCACGCTGCGGGTTGACCAGTCCATCCTGACAGGCGAGTCTGTATCTGTCATCAAACACACGGAGC |
| | CCGTTCCTGACCCCCGAGCTGTCAACCAGGACAAGAAGAACATGCTTTTCTCGGGCACCAACATTGC |
| | AGCCGGCAAGGCCTTGGGCATCGTGGCCACCACTGGTGTGGGCACCGAGATTGGGAAGATCCGAGAC |
| | CAAATGGCTGCCACAGAACAGGACAAGACCCCCTTGCAGCAGAAGCTGGATGAGTTTGGGGAGCAGC |
| | TCTCCAAGGTCATCTCCCTCATCTGTGTGGCTGTCTGGCTTATCAACATTGGCCACTTCAACGACCC |
| | CGTCCATGGGGGCTCCTGGTTCCGCGGGGCCATCTACTACTTTAAGATTGCCGTGGCCTTGGCTGTG |
| | GCTGCCATCCCCGAAGGTCTTCCTGCAGTCATCACCACCTGCCTGGCCCTGGGTACCCGTCGGATGG |
| | CAAAGAAGAATGCCATTGTAAGAAGCTTGCCCTCCGTAGAGACCCTGGGCTGCACCTCTGTCATCTG |
| | TTCCGACAAGACAGGCACCCTCACCACCAACCAGATGTCTGTCTGCAAGATGTTTATCATTGACAAG |
| | GTGGATGGGGACATCTGCCTCCTGAATGAGTTCTCCATCACCGGCTCCACTTACGCTCCAGAGGGAG |
| | AGGTCTTGAAGAATGATAAGCCAGTCCGGCCAGGGCAGTATGACGGGCTGGTGGAGCTGGCCACCAT |
| | CTGTGCCCTCTGCAATGACTCCTCCTTGGACTTCAACGAGGCCAAAGGTGTCTATGAGAAGGTCGGC |
| | GAGGCCACCGAGACAGCACTCACCACCCTGGTGGAGAAGATGAATGTGTTCAACACGGATGTGAGAA |
| | GCCTCTCGAAGGTGGAGAGAGCCAACGCCTGCAACTCGGTGATCCGCCAGCTAATGAAGAAGGAATT |
| | CACCCTGGAGTTCTCCCGAGACAGAAAGTCCATGTCTGTCTATTGCTCCCCAGCCAAATCTTCCCGG |
| | GCTGCTGTGGGCAACAAGATGTTTGTCAAGGGTGCCCCTGAGGGCGTCATCGACCGCTGTAACTATG |
| | TGCGAGTTGGCACCACCCGGGTGCCACTGACGGGGCCGGTGAAGGAAAAGATCATGGCGGTGATCAA |
| | GGAGTGGGGCACTGGCCGGGACACCCTGCGCTGCTTGGCCCTGGCCACCCGGGACACCCCCCCGAAG |
| | CGAGAGGAAATGGTCCTGGATGACTCTGCCAGGTTCCTGGAGTATGAGACGGACCTGACATTCGTGG |
| | GTGTAGTGGGCATGCTGGACCCTCCGCGCAAGGAGGTCACGGGCTCCATCCAGCTGTGCCGTGACGC |
| | CGGGATCCGGGTGATCATGATCACTGGGGACAACAAGGGCACAGCCATTGCCATCTGCCGGCGAATT |
| | GGCATCTTTGGGGAGAACGAGGAGGTGGCCGATCGCGCCTACACGGGCCGAGAGTTCGACGACCTGC |
| | CCCTGGCTGAACAGCGGGAAGCCTGCCGACGTGCCTGCTGCTTCGCCCGTGTGGAGCCCTCGCACAA |
| | GTCCAAGATTGTGGAGTACCTGCAGTCCTACGATGAGATCACAGCCATGACAGGTGATGGCGTCAAT |
| | GACGCCCCTGCCCTGAAGAAGGCTGAGATTGGCATTGCCATGGGATCTGGCACTGCCGTGGCCAAGA |
| | CTGCCTCTGAGATGGTGCTGGCTGACGACAACTTCTCCACCATCGTAGCTGCTGTGGAGGAGGGCCG |
| | CGCCATCTACAACAACATGAAGCAGTTCATCCGCTACCTCATTTCCTCCAACGTGGGCGAGGTGGTC |
| | TGTATCTTCCTGACCGCTGCCCTGGGGCTGCCTGAGGCCCTGATCCCGGTGCAGCTGCTATGGGTGA |
| | ACTTGGTGACCGACGGGCTCCCAGCCACAGCCCTGGGCTTCAACCCACCAGACCTGGACATCATGGA |
| | CCGCCCCCCCCGGAGCCCCAAGGAGCCCCTCATCAGTGGCTGGCTCTTCTTCCGCTACATGGCAATC |
| | GGGGGCTATGTGGGTGCAGCCACCGTGGGAGCAGCTGCCTGGTGGTTCCTGTACGCTGAGGATGGGC |
| | CTCATGTCAACTACAGCCAGCTGACTCACTTCATGCAGTGCACCGAGGACAACACCCACTTTGAGGG |
| | CATAGACTGTGAGGTCTTCGAGGCCCCCGAGCCCATGACCATGGCCCTGTCCGTGCTGGTGACCATC |
| | GAGATGTGCAATGCACTGAACAGCCTGTCCGAGAACCAGTCCCTGCTGCGGATGCCACCCTGGGTGA |
| | ACATCTGGCTGCTGGGCTCCATCTGCCTCTCCATGTCCCTGCACTTCCTCATCCTCTATGTTGACCC |
| | CCTGCCGATGATCTTCAAGCTCCGGGCCCTGGACCTCACCCAGTGGCTCATGGTCCTCAAGATCTCA |
| | CTGCCAGTCATTGGGCTCGACGAAATCCTCAAGTTCGTTGCTCGGAACTACCTAGAGGGATAA |

| | | |
|---|---|---|
| ORF Start: ATG at 6 | | ORF Stop: TAA at 3009 |
| SEQ ID NO: 274 | 1001 aa | MW at 110162.0 kD |

| | |
|---|---|
| NOV28l, | MGHHHHHHEAAHAKTTEECLAYFGVSETTGLTPDQVKRNLEKYGLNELPAEEGKTLWELVIEQFEDL |
| CG57417-05 | |
| Protein Sequence | LVRILLLAACISFVLAWFEEGEETITAFVEPFVILLILIANAIVGVWQERNAENAIEALKEYEPEMG |
| | KVYRADRKSVQRIKARDIVPGDIVEVAVGDKVPADIRILAIKSTTLRVDQSILTGESVSVIKHTEPV |
| | PDPRAVNQDKKNMLFSGTNIAAGKALGIVATTGVGTEIGKIRDQMAATEQDKTPLQQKLDEFGEQLS |
| | KVISLICVAVWLINIGHFNDPVHGGSWFRGAIYYFKIAVALAVAAIPEGLPAVITTCLALGTRRMAK |
| | KNAIVRSLPSVETLGCTSVICSDKTGTLTTNQMSVCKMFIIDKVDGDICLLNEFSITGSTYAPEGEV |
| | LKNDKPVRPGQYDGLVELATICALCNDSSLDFNEAKGVYEKVGEATETALTTLVEKMNVFNTDVRSL |
| | SKVERANACNSVIRQLMKKEFTLEFSRDRKSMSVYCSPAKSSRAAVGNKMFVKGAPEGVIDRCNYVR |
| | VGTTRVPLTGPVKEKIMAVIKEWGTGRDTLRCLALATRDTPPKREEMVLDDSARFLEYETDLTFVGV |
| | VGMLDPPRKEVTGSIQLCRDAGIRVIMITGDNKGTAIAICRRIGIFGENEEVADRAYTGREFDDLPL |
| | AEQREACRRACCFARVEPSHKSKIVEYLQSYDEITAMTGDGVNDAPALKKAEIGIAMGSGTAVAKTA |
| | SEMVLADDNFSTIVAAVEEGRAIYNNMKQFIRYLISSNVGEVVCIFLTAALGLPEALIPVQLLWVNL |
| | VTDGLPATALGFNPPDLDIMDRPPRSPKEPLISGWLFFRYMAIGGYVGAATVGAAAWWFLYAEDGPH |
| | VNYSQLTHFMQCTEDNTHFEGIDCEVFEAPEPMTMALSVLVTIEMCNALNSLSENQSLLRMPPWVNI |
| | WLLGSICLSMSLHFLILYVDPLPMIFKLRALDLTQWLMVLKISLPVIGLDEILKFVARNYLEG |

Sequence comparison of the above protein sequences yields the following sequence relationships shown in Table 28B.

TABLE 28B
Comparison of NOV28a against NOV28b through NOV28l.

| | NOV28a Residues/ | Identities/Similarities |
|---|---|---|
| Protein Sequence | Match Residues | for the Matched Region |
| NOV28b | 10 . . . 1009 | 1000/1000 (100%) |
| | 2 . . . 1001 | 1000/1000 (100%) |
| NOV28c | 10 . . . 1009 | 1000/1000 (100%) |
| | 3 . . . 1002 | 1000/1000 (100%) |
| NOV28d | 10 . . . 1001 | 987/992 (99%) |
| | 2 . . . 993 | 989/992 (99%) |
| NOV28e | 10 . . . 1001 | 992/992 (100%) |
| | 3 . . . 994 | 992/992 (100%) |
| NOV28f | 10 . . . 1009 | 1000/1000 (100%) |
| | 3 . . . 1002 | 1000/1000 (100%) |
| NOV28g | 10 . . . 1009 | 1000/1000 (100%) |
| | 3 . . . 1002 | 1000/1000 (100%) |
| NOV28h | 10 . . . 1009 | 1000/1000 (100%) |
| | 3 . . . 1002 | 1000/1000 (100%) |
| NOV28i | 10 . . . 1009 | 1000/1000 (100%) |
| | 2 . . . 1001 | 1000/1000 (100%) |
| NOV28j | 10 . . . 1009 | 1000/1000 (100%) |
| | 3 . . . 1002 | 1000/1000 (100%) |
| NOV28k | 10 . . . 1009 | 1000/1000 (100%) |
| | 3 . . . 1002 | 1000/1000 (100%) |
| NOV28l | 2 . . . 1001 | 1000/1000 (100%) |
| | 1 . . . 1000 | 1000/1000 (100%) |

Further analysis of the NOV28a protein yielded the following properties shown in Table 28C.

TABLE 28C
Protein Sequence Properties NOV28a

| SignalP | | |
|---|---|---|
| analysis: | No Known Signal Sequence Predicted | |
| PSORT II | PSG: | a new signal peptide prediction method |
| analysis: | | N-region: length 10; pos. chg 0; neg. chg 1 |
| | | H-region: length 4; peak value 0.00 |
| | | PSG Score: −4.40 |
| | GvH: | von Heijne's method for signal seq. recognition |
| | | GvH score (threshold: −2.1): −8.08 |
| | | possible cleavage site: between 31 and 32 |
| | >>> Seems to have no N-terminal signal peptide | |
| | ALOM: | Klein et al's method for TM region allocation |
| | | Init position for calculation: 1 |
| | | Tentative number of TMS(s) for the threshold 0.5: 8 |
| | | INTEGRAL Likelihood = −9.92 |
| | | Transmembrane 68—84 |
| | | INTEGRAL Likelihood = −8.33 |
| | | Transmembrane 96—112 |
| | | INTEGRAL Likelihood = −5.10 |
| | | Transmembrane 271—287 |
| | | INTEGRAL Likelihood = −3.19 |
| | | Transmembrane 306—322 |
| | | INTEGRAL Likelihood = −5.41 |
| | | Transmembrane 780—796 |
| | | INTEGRAL Likelihood = −0.00 |
| | | Tranemembrane 905—921 |
| | | INTEGRAL Likelihood = −3.13 |
| | | Transmembrane 939—955 |
| | | INTEGRAL Likelihood = −1.17 |
| | | Transmembrane 976—992 |
| | | PERIPHERAL Likelihood = 3.07 (at 846) |
| | | ALOM score: −9.92 (number ofTMSs: 8) |
| | MTOP: | Prediction of membrane topology (Hartmann et al.) |
| | | Center position for calculation: 75 |
| | | Charge difference: −2.0 C(−5.0) - N(−3.0) |
| | | N >= C: N-terminal side will be inside |

>>> membrane topology: type 3a
MITDISC: discrimination of mitochondrial targeting seq

| | | | |
|---|---|---|---|
| R content: | 0 | Hyd Moment | 2.28 |
| Hyd Moment (95): | 3.41 | (75): | |
| D/E content: | 2 | G content: | 1 |
| Score: | −7.46 | S/T content: | 3 |

| Gavel: | prediction of cleavage sites for mitochondrial preseq |
|---|---|
| | cleavage site motif not found |
| NUCDISC: discrimination of nuclear localization signals | |
| | pat4: none |
| | pat7: none |
| | bipartite: none |
| | content of basic residues: 10.3% |
| | NLS Score: −0.47 |
| KDEL: | ER retention motif in the C-terminus: none |
| ER Membrane Retention Signals: none | |
| SKL: | peroxisomal targeting signal in the C-terminus: none |
| PTS2: | 2nd peroxisomal targeting signal: found |
| | KLDEFGEQL at 260 |
| VAC: | possible vacuolar targeting motif: none |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

-----------------------------------
Final Results (k = 9/23):

44.4%: endoplasmic reticulum
22.2%: mitochondrial
11.1%: vacuolar
11.1%: Golgi
11.1%: vesicles of secretory system

>> prediction for CG57417-05 is end (k = 9)

A search of the NOV28a protein against the Geneseq database, a proprietary database that contains sequences published in patents and patent publication, yielded several homologous proteins shown in Table 28D.

TABLE 28D
Geneseq Results for NOV28a

| Geneseq | Protein/Organism/Length | NOV28a Residues/ | Identities/Similarities | Expect |
|---|---|---|---|---|
| Identifier | [Patent #, Date] | Match Residues | for the Matched Region | Value |
| AAM78337 | Human protein SEQ ID NO | 10 . . . 1009 | 1000/1000 (100%) | 0.0 |
| | 999 - Homo sapiens, 1001 | 2 . . . 1001 | 1000/1000 (100%) | |
| | aa. [WO200157190-A2, | | | |
| | 09-AUG-2001] | | | |
| AAM79321 | Human protein SEQ ID NO | 10 . . . 1009 | 999/1014 (98%) | 0.0 |
| | 2967 - Homo sapiens, 1072 | 59 . . . 1072 | 999/1014 (98%) | |
| | aa. [WO200157190-A2, | | | |
| | 09-AUG-2001] | | | |
| ABB09807 | Amino acid sequence of | 10 . . . 1001 | 992/992 (100%) | 0.0 |
| | human SERCA 1 - Homo | 2 . . . 993 | 992/992 (100%) | |
| | sapiens, 994 aa. | | | |
| | [WO200222777-A2, | | | |
| | 21-MAR-2002] | | | |
| AAB90764 | Human shear stress- | 10 . . . 1001 | 837/992 (84%) | 0.0 |
| | response protein SEQ ID | 2 . . . 992 | 924/992 (92%) | |
| | NO: 28 - Homo sapiens, | | | |
| | 997 aa. | | | |
| | [WO200125427-A1, | | | |
| | 12-APR-2001] | | | |
| ABB60125 | Drosophila melanogaster | 10 . . . 1004 | 711/995 (71%) | 0.0 |
| | polypeptide SEQ ID NO | 2 . . . 996 | 829/995 (82%) | |
| | 7167 - Drosophila | | | |
| | melanogaster, 1002 aa. | | | |
| | [WO200171042-A2, | | | |
| | 27-SEP-2001] | | | |

In a BLAST search of public sequence datbases, the NOV28a protein was found to have homology to the proteins shown in the BLASTP data in Table 28E.

TABLE 28E
Public BLASTP Results for NOV28a

| Protein | | | | |
|---|---|---|---|---|
| Accession | | NOV28a Residues/ | Identities/Similarities | Expect |
| Number | Protein/Organism/Length | Match Residues | for the Matched Portion | Value |
| O14983 | Sarcoplasmic/endoplasmic | 10 . . . 1009 | 1000/1000 (100%) | 0.0 |
| | reticulum calcium ATPase 1 | 2 . . . 1001 | 1000/1000 (100%) | |
| | (EC 3.6.3.8) (Calcium pump | | | |
| | 1) (SERCA1) (SR | | | |
| | Ca(2+)-ATPase 1) | | | |
| | (Calcium-transporting | | | |
| | ATPase sarcoplasmic | | | |
| | reticulum type, fast twitch | | | |
| | skeletal muscle isoform) | | | |
| | (Endoplasmic reticulum | | | |
| | class 1/2 Ca(2+) ATPase) - | | | |
| | Homo sapiens (Human), | | | |
| | 1001 aa. | | | |
| CAD34608 | Sequence 25 from Patent | 10 . . . 1001 | 992/992 (100%) | 0.0 |
| | WO0222777 - Homo | 2 . . . 993 | 992/992 (100%) | |
| | sapiens (Human), 994 aa. | | | |
| P04191 | Sarcoplasmic/endoplasmic | 10 . . . 1009 | 967/1000 (96%) | 0.0 |
| | reticulum calcium ATPase 1 | 2 . . . 1001 | 988/1000 (98%) | |
| | (EC 3.6.3.8) (Calcium pump | | | |
| | 1) (SERCA1) (SR | | | |
| | Ca(2+)-ATPase 1) | | | |
| | (Calcium-transporting | | | |
| | ATPase sarcoplasmic | | | |
| | reticulum type, fast twitch | | | |
| | skeletal muscle isoform) | | | |
| | (Endoplasmic reticulum | | | |
| | class 1/2 Ca(2+) ATPase) - | | | |
| | Oryctolagus cuniculus | | | |
| | (Rabbit), 1001 aa. | | | |
| Q64578 | Sarcoplasmic/endoplasmic | 10 . . . 1001 | 961/992 (96%) | 0.0 |
| | reticulum calcium ATPase 1 | 2 . . . 993 | 980/992 (97%) | |
| | (EC 3.6.3.8) (Calcium pump | | | |
| | 1) (SERCA1) (SR | | | |
| | Ca(2+)-ATPase 1) | | | |
| | (Calcium-transporting | | | |
| | ATPase sarcoplasmic | | | |
| | reticulum type, fast twitch | | | |
| | skeletal muscle isoform) | | | |
| | (Endoplasmic reticulum | | | |
| | class 1/2 Ca(2+) ATPase) - | | | |
| | Rattus norvegicus (Rat), 994 | | | |
| | aa. | | | |
| Q8R429 | Calcium-transporting | 10 . . . 1001 | 959/992 (96%) | 0.0 |
| | ATPase - Mus musculus | 2 . . . 993 | 979/992 (98%) | |
| | (Mouse), 994 aa. | | | |

PFam analysis predicts that the NOV28a protein contains the domains shown in the Table 28F.

TABLE 28F
Domain Analysis of NOV28a

| | | Identities/ | |
|---|---|---|---|
| | NOV28a Match | Similarities for | Expect |
| Pfam Domain | Region | the Matched Region | Value |
| Cation_ATPase_N | 11 . . . 85 | 26/87 (30%) | 2.9e—18 |
| | | 62/87 (71%) | |
| E1-E2_ATPase | 101 . . . 349 | 119/250 (48%) | 1.4e—124 |
| | | 225/250 (90%) | |
| Hydrolase | 353 . . . 732 | 45/385 (12%) | 1.5e—14 |
| | | 251/385 (65%) | |
| Cation_ATPase_C | 826 . . . 999 | 86/197 (44%) | 5.3e—84 |
| | | 161/197 (82%) | |

### Example 29

The NOV29 clone was analyzed, and the nucleotide and encoded polypeptide sequences are shown in Table 29A.

TABLE 29A
NOV29 Sequence Analysis

| | |
|---|---|
| SEQ ID NO: 275 | 2617 bp |

| | |
|---|---|
| NOV29a, | CACCATGGGCCACCATCACCACCATCACGCAAGGAGGAGCTCGTTCCAGTCGTGTCAGATAATATCC |
| CG93541-05 | |
| DNA Sequence | CTGTTCACTTTTGCCGTTGGAGTCAATATCTGCTTAGGATTCACTGCACATCGAATTAAGAGAGCAG |
| | AAGGATGGGAGGAAGGTCCTCCTACAGTGCTATCAGACTCCCCCTGGACCAACATCTCCGGATCTTG |
| | CAAGGGCAGGTGCTTTGAACTTCAAGAGGCTGGACCTCCTGATTGTCGCTGTGACAACTTGTGTAAG |
| | AGCTATACCAGTTGCTGCCATGACTTTGATGAGCTGTGTTTGAAGACAGCCCGTGGCTGGGAGTGTA |
| | CTAAGGACAGATGTGGAGAAGTCAGAAATGAAGAAAATGCCTGTCACTGCTCAGAGGACTGCTTGGC |
| | CAGGGGAGACTGCTGTACCAATTACCAAGTGGTTTGCAAAGGAGAGTCGCATTGGGTTGATGATGAC |
| | TGTGAGGAAATAAAGGCCGCAGAATGCCCTGCAGGGTTTGTTCGCCCTCCATTAATCATCTTCTCCG |
| | TGGATGGCTTCCGTGCATCATACATGAAGAAAGGCAGCAAAGTCATGCCTAATATTGAAAAACTAAG |
| | GTCTTGTGGCACACACTCTCCCTACATGAGGCCGGTGTACCCAACTAAAACCTTTCCTAACTTATAC |
| | ACTTTGGCCACTGGGCTATATCCAGAATCACATGGAATTGTTGGCAATTCAATGTATGATCCTGTAT |
| | TTGATGCCACTTTTCATCTGCGAGGGCGAGAGAAATTTAATCATAGATGGTGGGGAGGTCAACCGCT |
| | ATGGATTACAGCCACCAAGCAAGGGGTGAAAGCTGGAACATTCTTTTGGTCTGTTGTCATCCCTCAC |
| | GAGCGGAGAATATTAACCATATTGCAGTGGCTCACCCTGCCAGATCATGAGAGGCCTTCGGTCTATG |
| | CCTTCTATTCTGAGCAACCTGATTTCTCTGGACACAAATATGGCCCTTTCGGCCCTGAGATGACAAA |
| | TCCTCTGAGGGAAATCGACAAAATTGTGGGGCAATTAATGGATGGACTGAAACAACTAAAACTGCAT |
| | CGGTGTGTCAACGTCATCTTTGTCGGAGACCATGGAATGGAAGATGTCACATGTGATAGAACTGAGT |
| | TCTTGAGTAATTACCTAACTAATGTGGATGATATTACTTTAGTGCCTGGAACTCTAGGAAGAATTCG |
| | ATCCAAATTTAGCAACAATGCTAAATATGACCCCAAAGCCATTATTGCCAATCTCACGTGTAAAAAA |
| | CCAGATCAGCACTTTAAGCCTTACTTGAAACAGCACCTTCCCAAACGTTTGCACTATGCCAACAACA |
| | GAAGAATTGAGGATATCCATTTATTGGTGGAACGCAGATGGCATGTTGCAAGGAAACCTTTGGATGT |
| | TTATAAGAAACCATCAGGAAAATGCTTTTTCCAGGGAGACCACGGATTTGATAACAAGGTCAACAGC |
| | ATGCAGACTGTTTTTGTAGGTTATGGCCCAACATTTAAGTACAAGACTAAAGTGCCTCCATTTGAAA |
| | ACATTGAACTTTACAATGTTATGTGTGATCTCCTGGGATTGAAGCCAGCTCCTAATAATGGGACCCA |
| | CGGAAGTTTGAATCATCTCCTGCGCACTAATACCTTCAGGCCAACCATGCCAGAGGAAGTTACCAGA |
| | CCCAATTATCCAGGGATTATGTACCTTCAGTCTGATTTTGACCTGGGCTGCACTTGTGATGATAAGG |
| | TAGAGCCAAAGAACAAGTTGGATGAACTCAACAAACGGCTTCATACAAAAGGGTCTACAGAAGAGAG |
| | ACACCTCCTCTATGGGCGACCTGCAGTGCTTTATCGGACTAGATATGATATCTTATATCACACTGAC |
| | TTTGAAAGTGGTTATAGTGAAATATTCCTAATGCCACTCTGGACATCATATACTGTTTCCAAACAGG |
| | CTGAGGTTTCCAGCGTTCCTGACCATCTGACCAGTTGCGTCCGGCCTGATGTCCGTGTTTCTCCCAG |
| | TTTCAGTCAGAACTGTTTGGCCTACAAAAATGATAAGCAGATGTCCTACGGATTCCTCTTTCCTCCT |
| | TATCTGAGCTCTTCACCAGAGGCTAAATATGATGCATTCCTTGTAACCAATATGGTTCCAATGTATC |
| | CTGCTTTCAAACGGGTCTGGAATTATTTCCAAAGGGTATTGGTGAAGAAATATGCTTCGGAAAGAAA |
| | TGGAGTTAACGTGATAAGTGGACCAATCTTCGACTATGACTATGATGGCTTACATGACACAGAAGAC |
| | AAAATAAAACAGTACGTGGAAGGCAGTTCCATTCCTGTTCCAACTCACTACTACAGCATCATCACCA |
| | GCTGTCTGGATTTTACTCAGCCTGCCGACAAGTGTGACGGCCCTCTCTCTGTGTCCTCCTTCATCCT |
| | GCCTCACCGGCCTGACAACGAGGAGAGCTGCAATAGCTCAGAGGACGAATCAAAATGGGTAGAAGAA |
| | CTCATGAAGATGCACACAGCTAGGGTGCGTGACATTGAACATCTCACCAGCCTGGACTTCTTCCGAA |
| | AGACCAGCCGCAGCTACCCAGAAATCCTGACACTCAAGACATACTTGCATACATATGAGAGCGAGAT |
| | TTAA |

| | | |
|---|---|---|
| ORF Start: at 2 | | ORF Stop: TAA at 2615 |
| SEQ ID NO: 276 | 871 aa | MW at 99983.6 kD |

| | |
|---|---|
| NOV29a, | TMGHHHHHHARRSSFQSCQIISLFTFAVGVNICLGFTAHRIKRAEGWEEGPPTVLSDSPWTNISGSC |
| CG93541-05 | |
| Protein Sequence | KGRCFELQEAGPPDCRCDNLCKSYTSCCHDFDELCLKTARGWECTKDRCGEVRNEENACHCSEDCLA |
| | RGDCCTNYQVVCKGESHWVDDDCEEIKAAECPAGFVRPPLIIFSVDGFRASYMKKGSKVMPNIEKLR |
| | SCGTHSPYMRPVYPTKTFPNLYTLATGLYPESHGIVGNSMYDPVFDATFHLRGREKFNHRWWGGQPL |
| | WITATKQGVKAGTFFWSVVIPHERRILTILQWLTLPDHERPSVYAFYSEQPDFSGHKYGPFGPEMTN |
| | PLREIDKIVGQLMDGLKQLKLHRCVNVIFVGDHGMEDVTCDRTEFLSNYLTNVDDITLVPGTLGRIR |
| | SKFSNNAKYDPKAIIANLTCKKPDQHFKPYLKQHLPKRLHYANNRRIEDIHLLVERRWHVARKPLDV |
| | YKKPSGKCFFQGDHGFDNKVNSMQTVFVGYGPTFKYKTKVPPFENIELYNVMCDLLGLKPAPNNGTH |
| | GSLNHLLRTNTFRPTMPEEVTRPNYPGIMYLQSDFDLGCTCDDKVEPKNKLDELNKRLHTKGSTEER |
| | HLLYGRPAVLYRTRYDILYHTDFESGYSEIFLMPLWTSYTVSKQAEVSSVPDHLTSCVRPDVRVSPS |
| | FSQNCLAYKNDKQMSYGFLFPPYLSSSPEAKYDAFLVTNMVPMYPAFKRVWNYFQRVLVKKYASERN |
| | GVNVISGPIFDYDYDGLHDTEDKIKQYVEGSSIPVPTHYYSIITSCLDFTQPADKCDGPLSVSSFIL |
| | PHRPDNEESCNSSEDESKWVEELMKMHTARVRDIEHLTSLDFFRKTSRSYPEILTLKTYLHTYESEI |

| | |
|---|---|
| SEQ ID NO:277 | 3110 bp |

| | |
|---|---|
| NOV29b, | AGTGCACTCCGTGAAGGCAAAGAGAACACGCTGCAAAAGGCTTTCCAATAATCCTCGACATGGCAAG |
| CG93541-01 | |
| DNA Sequence | GAGGAGCTCGTTCCAGTCGTGTCAGATAATATCCCTGTTCACTTTTGCCGTTGGAGTCAATATCTGC |
| | TTAGGATTCACTGCACATCGAATTAAGAGAGCAGAAGGATGGGAGGAAGGTCCTCCTACAGTGCTAT |
| | CAGACTCCCCCTGGACCAACATCTCCGGATCTTGCAAGGGCAGGTGCTTTGAACTTCAAGAGGCTGG |
| | ACCTCCTGATTGTCGCTGTGACAACTTGTGTAAGAGCTATACCAGTTGCTGCCATGACTTTGATGAG |
| | CTGTGTTTGAAGACAGCCCGTGCGTGGGAGTGTACTAAGGACAGATGTGGGGAAGTCAGAAATGAAG |
| | AAAATGCCTGTCACTGCTCAGAGGACTGCTTGGCCAGGGGAGACTGCTGTACCAATTACCAAGTGGT |
| | TTGCAAAGGAGAGTCGCATTGGGTTGATGATGACTGTGAGGAAATAAAGGCCGCAGAATGCCCTGCA |
| | GGGTTTGTTCGCCCTCCATTAATCATCTTCTCCGTGGATGGCTTCCGTGCATCATACATGAAGAAAG |
| | GCAGCAAAGTCATGCCTAATATTGAAAAACTAAGGTCTTGTGGCACACACTCTCCCTACATGAGGCC |
| | GGTGTACCCAACTAAAACCTTTCCTAACTTATACACTTTGGCCACTGGGCTATATCCAGAATCACAT |
| | GGAATTGTTGGCAATTCAATGTATGATCCTGTATTTGATGCCACTTTTCATCTGCGAGGGCGAGAGA |
| | AATTTAATCATAGATGGTGGGGAGGTCAACCGCTATGGATTACAGCCACCAAGCAAGGGGTGAAAGC |
| | TGGAACATTCTTTTGGTCTGTTGTCATCCCTCACGAGCGGAGAATATTAACCATATTGCAGTGGCTC |
| | ACCCTGCCAGATCATGAGAGGCCTTCGGTCTATGCCTTCTATTCTGAGCAACCTGATTTCTCTGGAC |
| | ACAAATATGGCCCTTTCGGCCCTGAGATGACAAATCCTCTGAGGGAAATCGACAAAATTGTGGGGCA |
| | ATTAATGGATGGACTGAAACAACTAAAACTGCATCGGTGTGTCAACGTCATCTTTGTCGGAGACCAT |
| | GGAATGGAAGATGTCACATGTGATAGAACTGAGTTCTTGAGTAATTACCTAACTAATGTGGATGATA |
| | TTACTTTAGTGCCTGGAACTCTAGGAAGAATTCGATCCAAATTTAGCAACAATGCTAAATATGACCC |
| | CAAAGCCATTATTGCCAATCTCACGTGTAAAAAACCAGATCAGCACTTTAAGCCTTACTTGAAACAG |
| | CACCTTCCCAAACGTTTGCACTATGCCAACAACAGAAGAATTGAGGATATCCATTTATTGGTGGAAC |
| | GCAGATGGCATGTTGCAAGGAAACCTTTGGATGTTTATAAGAAACCATCAGGAAAATGCTTTTTCCA |
| | GGGAGACCACGGATTTGATAACAAGGTCAACAGCATGCAGACTGTTTTTGTAGGTTATGGCCCAACA |
| | TTTAAGTACAAGACTAAAGTGCCTCCATTTGAAAACATTGAACTTTACAATGTTATGTGTGATCTCC |
| | TGGGATTGAAGCCAGCTCCTAATAATGGGACCCATGGAAGTTTGAATCATCTCCTGCGCACTAATAC |
| | CTTCAGGCCAACCATGCCAGAGGAAGTTACCAGACCCAATTATCCAGGGATTATGTACCTTCAGTCT |
| | GATTTTGACCTGGGCTGCACTTGTGATGATAAGGTAGAGCCAAAGAACAAGTTGGATGAACTCAACA |
| | AACGGCTTCATACAAAAGGGTCTACAGAAGAGAGACACCTCCTCTATGGGCGACCTGCAGTGCTTTA |
| | TCGGACTAGATATGATATCTTATATCACACTGACTTTGAAAGTGGTTATAGTGAAATATTCCTAATG |
| | CCACTCTGGACATCATATACTGTTTCCAAACAGGCTGAGGTTTCCAGCGTTCCTGACCATCTGACCA |
| | GTTGCGTCCGGCCTGATGTCCGTGTTTCTCCGAGTTTCAGTCAGAACTGTTTGGCCTACAAAAATGA |
| | TAAGCAGATGTCCTACGGATTCCTCTTTCCTCCTTATCTGAGCTCTTCACCAGAGGCTAAATATGAT |
| | GCATTCCTTGTAACCAATATGGTTCCAATGTATCCTGCTTTCAAACGGGTCTGGAATTATTTCCAAA |
| | GGGTATTGGTGAAGAAATATGCTTCGGAAAGAAATGGAGTTAACGTGATAAGTGGACCAATCTTCGA |
| | CTATGACTATGATGGCTTACATGACACAGAAGACAAAATAAAACAGTACGTGGAAGGCAGTTCCATT |
| | CCTGTTCCAACTCACTACTACAGCATCATCACCAGCTGTCTGGATTTCACTCAGCCTGCCGACAAGT |
| | GTGACGGCCCTCTCTCTGTGTCCTCCTTCATCCTGCCTCACCGGCCTGACAACGAGGAGAGCTGCAA |
| | TAGCTCAGAGGACGAATCAAAATGGGTAGAAGAACTCATGAAGATGCACACAGCTAGGGTGCGTGAC |
| | ATTGAACATCTCACCAGCCTGGACTTCTTCCGAAAGACCAGCCGCAGCTACCCAGAAATCCTGACAC |
| | TCAAGACATACCTGCATACATATGAGAGCGAGATTTAACTTTCTGAGCATCTGCAGTACAGTCTTAT |
| | CAACTGGTTGTATATTTTTATATTGTTTTTGTATTTATTAATTTGAAACCAGGACATTAAAAATGTT |
| | AGTATTTTAATCCTGTACCAAATCTGACATATTATGCCTGAATGACTCCACTGTTTTTCTCTAATGC |
| | TTGATTTAGGTAGCCTTGTGTTCTGAGTAGAGCTTGTAATAAATACTGCAGCTTGAGTTTTTAGTGG |
| | AAGCTTCTAAATGGTGCTGCAGATTTGATATTTGCATTGAGGAAATATTAATTTTCCAATGCACAGT |
| | TGCCACATTTAGTCCTGTACTGTATGGAAACACTGATTTTGTAAAGTTGCCTTTATTTGCTGTTAAC |
| | TGTTAACTATGACAGATATATTTAAGCCTTATAAACCAATCTTAAACATAATAAATCACACATTCAG |
| | TTTTTTCTGGTAAAAAAAAAAAAAAAAA |

| | | |
|---|---|---|
| ORF Start: ATG at 60 | | ORF Stop: TAA at 2649 |
| SEQ ID NO: 278 | 863 aa | MW at 99016.6 kD |

| | |
|---|---|
| NOV29b, | MARRSSFQSCQIISLFTFAVGVNICLGFTAHRIKRAEGWEEGPPTVLSDSPWTNISGSCKGRCFELQ |
| CG93541-01 | |
| Protein Sequence | EAGPPDCRCDNLCKSYTSCCHDFDELCLKTARAWECTKDRCGEVRNEENACHCSEDCLARGDCCTNY |
| | QVVCKGESHWVDDDCEEIKAAECPAGFVRPPLIIFSVDGFRASYMKKGSKVMPNIEKLRSCGTHSPY |
| | MRPVYPTKTFPNLYTLATGLYPESHGIVGNSMYDPVFDATFHLRGREKFNHRWWGGQPLWITATKQG |
| | VKAGTFFWSVVIPHERRILTILQWLTLPDHERPSVYAFYSEQPDFSGHKYGPFGPEMTNPLREIDKI |
| | VGQLMDGLKQLKLHRCVNVIFVGDHGMEDVTCDRTEFLSNYLTNVDDITLVPGTLGRIRSKFSNNAK |
| | YDPKAIIANLTCKKPDQHFKPYLKQHLPKRLHYANNRRIEDIHLLVERRWHVARKPLDVYKKPSGKC |
| | FFQGDHGFDNKVNSMQTVFVGYGPTFKYKTKVPPFENIELYNVMCDLLGLKPAPNNGTHGSLNHLLR |
| | TNTFRPTMPEEVTRPNYPGIMYLQSDPDLGCTCDDKVEPKNKLDELNKRLHTKGSTEERHLLYGRPA |
| | VLYRTRYDILYHTDFESGYSEIFLMPLWTSYTVSKQAEVSSVPDHLTSCVRPDVRVSPSFSQNCLAY |
| | KNDKQMSYGFLFPPYLSSSPEAKYDAFLVTNMVPMYPAFKRVWNYFQRVLVKKYASERNGVNVISGP |
| | IFDYDYDGLHDTEDKIKQYVEGSSIPVPTHYYSIITSCLDFTQPADKCDGPLSVSSFILPHRPDNEE |
| | SCNSSEDESKWVEELMKMHTARVRDIEHLTSLDFFRKTSRSYPEILTLKTYLHTYESEI |

| | |
|---|---|
| SEQ ID NO: 279 | 1080 bp |

| | |
|---|---|
| NOV29c, | CGTGAAGGCAAAGAGAACACGCTGCAAAAGGCTTCCAAGAATCCTCGACATGGCAAGAAGGAGCTCG |
| CG93541-02 | |
| DNA Sequence | TTCCAGTCGTGTCAGATAATATCCCTGTTCACTTTTGCCGTTGGAGTCAATATCTGCTTAGGATTCA |
| | CTGCACATCGAATTAAGAGAGCAGAAGGATGGGAGGAAGGTCCTCCTACAGTGCTATCAGACTCCCC |
| | CTGGACCAACATCTCCGGATCTTGCAAGGGCAGGTGCTTTGAACTTCAAGAGGCTGGACCTCCTGAT |
| | TGTCGCTGTGACAACTTGTGTAAGAGCTATACCAGTTGCTGCCATGACTTTGATGAGCTGTGTTTGA |
| | AGACAGCCCGTGGCTGGGAGTGTACTAAGGACAGATGTGGAGAAGTCAGAAATGAAGAAAATGCCTG |
| | TCACTGCTCAGAGGACTGCTTGGCCAGGGGAGACTGCTGTACCAATTACCAAGTGGTTTGCAAAGGA |
| | GAGTCGCATTGGGTTGATGATGACTGTGAGGAAATAAAGGCCGCAGAATGCCCTGCAGGGTTTGTTC |
| | GCCCTCCATTAATCATCTTCTCCGTGGATGGCTTCCGAAAGACCAGCCGCAGCTACCCAGAAATCCT |
| | GACACTCAAGACATACCTGCATACATATGAGAGCGAGATTTAACTTTCTGAGCATCTGCAGTACAGT |
| | CTTATCAACTGGTTGTATATTTTTATATTGTTTTTGTATTTATTAATTTGAAACCAGGACATTAAAA |
| | ATGTTAGTATTTTAATCCTGTACCAAATCTGACATATTATGCCTGAATGACTCCACTGTTTTTCTCT |
| | AATGCTTGATTTAGGTAGCCTTGTGTTCTGAGTAGAGCTTGTAATAAATACTGCAGCTTGAGAAAAA |
| | GTGGAAGCTTCTAAATGGTGCTGCAGATTTGATATTTGCATTGAGGAAATATTAATTTTCCAATGCA |
| | CAGTTGCCACATTTAGTCCTGTACTGTATGGAAACACTGATTTTGTAAAGTTGCCTTTATTTGCTGT |
| | TAACTGTTAACTATGACAGATATATTTAAGCCTTATAAACCAATCTTAAACATAATAAATCACACAT |
| | TCAGTTTT |

| | | |
|---|---|---|
| ORF Start: ATG at 50 | | ORF Stop: TAA at 644 |
| SEQ ID NO: 280 | 198 aa | MW at 22254.8 kD |

| | |
|---|---|
| NOV29c, | MARRSSFQSCQIISLFTFAVGVNICLGFTAHRIKRAEGWEEGPPTVLSDSPWTNISGSCKGRCFELQ |
| CG93541-02 | |
| Protein Sequence | EAGPPDCRCDNLCKSYTSCCHDFDELCLKTARGWECTKDRCGEVRNEENACHCSEDCLARGDCCTNY |
| | QVVCKGESHWVDDDCEEIKAAECPAGFVRPPLIIFSVDGFRKTSRSYPEILTLKTYLHTYESEI |

| | |
|---|---|
| SEQ ID NO: 281 | 2511 bp |

| | |
|---|---|
| NOV29d, | ACCATGGTAAGCGCTATTGTTTTATATGTGCTTTTGGCGGCGGCGGCGCATTCTGCCTTTGCGGACT |
| CG93541-03 | |
| DNA Sequence | CCCCCTGGACCAACATCTCCGGATCTTGCAAGGGCAGGTGCTTTGAACTTCAAGAGGCTGGACCTCC |
| | TGATTGTCGCTGTGACAACTTGTGTAAGAGCTATACCAGTTGCTGCCATGACTTTGATGAGCTGTGT |
| | TTGAAGACAGCCCGTGGCTGGGAGTGTACTAAGGACAGATGTGGAGAAGTCAGAAATGAAGAAAATG |
| | CCTGTCACTGCTCAGACGACTGCTTGGCCAGGGGAGACTGCTGTACCAATTACCAAGTGGTTTGCAA |
| | AGGAGAGTCGCATTGGGTTGATGATGACTGTGAGGAAATAAAGGCCGCAGAATGCCCTGCAGGGTTT |
| | GTTCGCCCTCCATTAATCATCTTCTCCGTGGATGGCTTCCGTGCATCATACATGAAGAAAGGCAGCA |
| | AAGTCATGCCTAATATTGAAAAACTAAGGTCTTGTGGCACACACTCTCCCTACATGAGGCCGGTGTA |
| | CCCAACTAAAACCTTTCCTAACTTATACACTTTGGCCACTGGGCTATATCCAGAATCACATGGAATT |
| | GTTGGCAATTCAATGTATGATCCTGTATTTGATGCCACTTTTCATCTGCGAGGGCGAGAGAAATTTA |
| | ATCATAGATGGTGGGGAGGTCAACCGCTATGGATTACAGCCACCAAGCAAGGGGTGAAAGCTGGAAC |
| | ATTCTTTTGGTCTGTTGTCATCCCTCACGAGCGGAGAATATTAACCATATTGCAGTGGCTCACCCTG |
| | CCAGATCATGAGAGGCCTTCGGTCTATGCCTTCTATTCTGAGCAACCTGATTTCTCTGGACACAAAT |
| | ATGGCCCTTTCGGCCCTGAGATGACAAATCCTCTGAGGGAAATCGACAAAATTGTGGGGCAATTAAT |
| | GGATGGACTGAAACAACTAAAACTGCATCGGTGTGTCAACGTCATCTTTGTCGGAGACCATGGAATG |
| | GAAGATGTCACATGTGATAGAACTGAGTTCTTGAGTAATTACCTAACTAATGTGGATGATATTACTT |
| | TAGTGCCTGGAACTCTAGGAAGAATTCGATCCAAATTTAGCAACAATGCTAAATATGACCCCAAAGC |
| | CATTATTGCCAATCTCACGTGTAAAAAACCAGATCAGCACTTTAAGCCTTACTTGAAACAGCACCTT |
| | CCCAAACGTTTGCACTATGCCAACAACAGAAGAATTGAGGATATCCATTTATTGGTGGAACGCAGAT |
| | GGCATGTTGCAAGGAAACCTTTGGATGTTTATAAGAAACCATCAGGAAAATGCTTTTTCCAGGGAGA |
| | CCACGGATTTGATAACAAGGTCAACAGCATGCAGACTGTTTTTGTAGGTTATGGCCCAACATTTAAG |
| | TACAAGACTAAAGTGCCTCCATTTGAAAACATTGAACTTTACAATGTTATGTGTGATCTCCTGGGAT |
| | TGAAGCCAGCTCCTAATAATGGGACCCACGGAAGTTTGAATCATCTCCTGCGCACTAATACCTTCAG |
| | GCCAACCATGCCAGAGGAAGTTACCAGACCCAATTATCCAGGGATTATGTACCTTCAGTCTGATTTT |
| | GACCTGGGCTGCACTTGTGATGATAAGGTAGAGCCAAAGAACAAGTTGGATGAACTCAACAAACGGC |
| | TTCATACAAAAGGGTCTACAGAAGAGAGACACCTCCTCTATGGGCGACCTGCAGTGCTTTATCGGAC |
| | TAGATATGATATCTTATATCACACTGACTTTGAAAGTGGTTATAGTGAAATATTCCTAATGCCACTC |
| | TGGACATCATATACTGTTTCCAAACAGGCTGAGGTTTCCAGCGTTCCTGACCATCTGACCAGTTGCG |
| | TCCGGCCTGATGTCCGTGTTTCTCCGAGTTTCAGTCAGAACTGTTTGGCCTACAAAAATGATAAGCA |
| | GATGTCCTACGGATTCCTCTTTCCTCCTTATCTGAGCTCTTCACCAGAGGCTAAATATGATGCATTC |
| | CTTGTAACCAATATGGTTCCAATGTATCCTGCTTTCAAACGGGTCTGGAATTATTTCCAAAGGGTAT |
| | TGGTGAAGAAATATGCTTCGGAAAGAAATGGAGTTAACGTGATAAGTGGACCAATCTTCGACTATGA |
| | CTATGATGGCTTACATGACACAGAAGACAAAATAAAACAGTACGTGGAAGGCAGTTCCATTCCTGTT |
| | CCAACTCACTACTACAGCATCATCACCAGCTGTCTGGATTTTACTCAGCCTGCCGACAAGTGTGACG |
| | GCCCTCTCTCTGTGTCCTCCTTCATCCTGCCTCACCGGCCTGACAACGAGGAGAGCTGCAATAGCTC |
| | AGAGGACGAATCAAAATGGGTAGAAGAACTCATGAAGATGCACACAGCTAGGGTGCGTGACATTGAA |
| | CATCTCACCAGCCTGGACTTCTTCCGAAAGACCAGCCGCAGCTACCCAGAAATCCTGACACTCAAGA |
| | CATACTTGCATACATATGAGAGCGAGATTTAA |

| | | |
|---|---|---|
| ORF Start: at 10 | | ORF Stop: at 835 |
| SEQ ID NO: 282 | 278 aa | MW at 31297.3 kD |

| | |
|---|---|
| NOV29d, | TMVSAIVLYVLLAAAAHSAFADSPWTNISGSCKGRCFELQEAGPPDCRCDNLCKSYTSCCHDFDELC |
| CG93541-03 | |
| Protein Sequence | LKTARGWECTKDRCGEVRNEENACHCSEDCLARGDCCTNYQVVCKGESHWVDDDCEEIKAAECPAGF |
| | VRPPLIIFSVDGFRASYMKKGSKVMPNIEKLRSCGTHSPYMRPVYPTKTFPNLYTLATGLYPESHGI |
| | VGNSMYDPVFDATFHLRGREKFNHRWWGGQPLWITATKQGVKAGTFFWSVVIPHERRILTILQWLTL |
| | PDHERPSVYA |

| | |
|---|---|
| SEQ ID NO: 283 | 2596 bp |

| | |
|---|---|
| NOV29e, | CCACCATGGCAAGGAGGAGCTCGTTCCAGTCGTGTCAGATAATATCCCTGTTCACTTTTGCCGTTGG |
| CG93541-04 | |
| DNA Sequence | AGTCAATATCTGCTTAGGATTCACTGCACATCGAATTAAGAGAGCAGAAGGATGGGAGGAAGGTCCT |
| | CCTACAGTGCTATCAGACTCCCCCTGGACCAACATCTCCGGATCTTGCAAGGGCAGGTGCTTTGAAC |
| | TTCAAGAGGCTGGACCTCCTGATTGTCGCTGTGACAACTTGTGTAAGAGCTATACCAGTTGCTGCCA |
| | TGACTTTGATGAGCTGTGTTTGAAGACAGCCCGTGGCTGGGAGTGTACTAAGGACAGATGTGGAGAA |
| | GTCAGAAATGAAGAAAATGCCTGTCACTGCTCAGAGGACTGCTTGGCCAGGGGAGACTGCTGTACCA |
| | ATTACCAAGTGGTTTGCAAAGGAGAGTCGCATTGGGTTGATGATGACTGTGAGGAAATAAAGGCCGC |
| | AGAATGCCCTGCAGGGTTTGTTCGCCCTCCATTAATCATCTTCTCCGTGGATGGCTTCCGTGCATCA |
| | TACATGAAGAAAGGCAGCAAAGTCATGCCTAATATTGAAAAACTAAGGTCTTGTGGCACACACTCTC |
| | CCTACATGAGGCCGGTGTACCCAACTAAAACCTTTCCTAACTTATACACTTTGGCCACTGGGCTATA |
| | TCCAGAATCACATGGAATTGTTGGCAATTCAATGTATGATCCTGTATTTGATGCCACTTTTCATCTG |
| | CGAGGGCGAGAGAAATTTAATCATAGATGGTGGGGAGGTCAACCGCTATGGATTACAGCCACCAAGC |
| | AAGGGGTGAAAGCTGGAACATTCTTTTGGTCTGTTGTCATCCCTCACGAGCGGAGAATATTAACCAT |
| | ATTGCAGTGGCTCACCCTGCCAGATCATGAGAGGCCTTCGGTCTATGCCTTCTATTCTGAGCAACCT |
| | GATTTCTCTGGACACAAATATGGCCCTTTCGGCCCTGAGATGACAAATCCTCTGAGGGAAATCGACA |
| | AAATTGTGGGGCAATTAATGGATGGACTGAAACAACTAAAACTGCATCGGTGTGTCAACGTCATCTT |
| | TGTCGGAGACCATGGAATGGAAGATGTCACATGTGATAGAACTGAGTTCTTGAGTAATTACCTAACT |
| | AATGTGGATGATATTACTTTAGTGCCTGGAACTCTAGGAAGAATTCGATCCAAATTTAGCAACAATG |
| | CTAAATATGACCCCAAAGCCATTATTGCCAATCTCACGTGTAAAAAACCAGATCAGCACTTTAAGCC |
| | TTACTTGAAACAGCACCTTCCCAAACGTTTGCACTATGCCAACAACAGAAGAATTGAGGATATCCAT |
| | TTATTGGTGGAACGCAGATGGCATGTTGCAAGGAAACCTTTGGATGTTTATAAGAAACCATCAGGAA |
| | AATGCTTTTTCCAGGGAGACCACGGATTTGATAACAAGGTCAACAGCATGCAGACTGTTTTTGTAGG |
| | TTATGGCCCAACATTTAAGTACAAGACTAAAGTGCCTCCATTTGAAAACATTGAACTTTACAATGTT |
| | ATGTGTGATCTCCTGGGATTGAAGCCAGCTCCTAATAATGGGACCCACGGAAGTTTGAATCATCTCC |
| | TGCGCACTAATACCTTCAGGCCAACCATGCCAGAGGAAGTTACCAGACCCAATTATCCAGGGATTAT |
| | GTACCTTCAGTCTGATTTTGACCTGGGCTGCACTTGTGATGATAAGGTAGAGCCAAAGAACAAGTTG |
| | GATGAACTCAACAAACGGCTTCATACAAAAGGGTCTACAGAAGAGAGACACCTCCTCTATGGGCGAC |
| | CTGCAGTGCTTTATCGGACTAGATATGATATCTTATATCACACTGACTTTGAAAGTGGTTATAGTGA |
| | AATATTCCTAATGCCACTCTGGACATCATATACTGTTTCCAAACAGGCTGAGGTTTCCAGCGTTCCT |
| | GACCATCTGACCAGTTGCGTCCGGCCTGATGTCCGTGTTTCTCCGAGTTTCAGTCAGAACTGTTTGC |
| | CCTACAAAAATGATAAGCAGATGTCCTACGGATTCCTCTTTCCTCCTTATCTGAGCTCTTCACCAGA |
| | GGCTAAATATGATGCATTCCTTGTAACCAATATGGTTCCAATGTATCCTGCTTTCAAACGGGTCTGG |
| | AATTATTTCCAAAGGGTATTGGTGAAGAAATATGCTTCGGAAAGAAATGGAGTTAACGTGATAAGTG |
| | GACCAATCTTCGACTATGACTATGATGGCTTACATGACACAGAAGACAAAATAAAACAGTACGTGGA |
| | AGGCAGTTCCATTCCTGTTCCAACTCACTACTACAGCATCATCACCAGCTGTCTGGATTTCACTCAG |
| | CCTGCCGACAAGTGTGACGGCCCTCTCTCTGTGTCCTCCTTCATCCTGCCTCACCGGCCTGACAACG |
| | AGGAGAGCTGCAATAGCTCAGAGGACGAATCAAAATGGGTAGAAGAACTCATGAAGATGCACACAGC |
| | TAGGGTGCGTGACATTGAACATCTCACCAGCCTGGACTTCTTCCGAAAGACCAGCCGCAGCTACCCA |
| | GAAATCCTGACACTCAAGACATACTTGCATACATATGGAGCGAGATTTAA |

| | | |
|---|---|---|
| ORF Start: at 3 | | ORF Stop: at 2595 |
| SEQ ID NO: 284 | 864 aa | MW at 99076.7 kD |

| | |
|---|---|
| NOV29e, | TMARRSSFQSCQIISLFTFAVGVNICLGFTAHRIKRAEGWEEGPPTVLSDSPWTNISGSCKGRCFEL |
| CG93541-04 | |
| Protein Sequence | QEAGPPDCRCDNLCKSYTSCCHDFDELCLKTARGWECTKDRCGEVRNEENACHCSEDCLARGDCCTN |
| | YQVVCKGESHWVDDDCEEIKAAECPAGFVRPPLIIFSVDGFRASYMKKGSKVMPNIEKLRSCGTHSP |
| | YMRPVYPTKTFPNLYTLATGLYPESHGIVGNSMYDPVFDATFHLRGREKFNHRWWGGQPLWITATKQ |
| | GVKAGTFFWSVVIPHERRILTILQWLTLPDHERPSVYAFYSEQPDFSGHKYGPFGPEMTNPLREIDK |
| | IVGQLMDGLKQLKLHRCVNVIFVGDHGMEDVTCDRTEFLSNYLTNVDDITLVPGTLGRIRSKFSNNA |
| | KYDPKAIIANLTCKKPDQHFKPYLKQHLPKRLHYANNRRIEDIHLLVERRWHVARKPLDVYKKPSGK |
| | CFFQGDHGFDNKVNSMQTVFVGYGPTFKYKTKVPPFENIELYNVMCDLLGLKPAPNNGTHGSLNHLL |
| | RTNTFRPTMPEEVTRPNYPGIMYLQSDFDLGCTCDDKVEPKNKLDELNKRLHTKGSTEERHLLYGRP |
| | AVLYRTRYDILYHTDFESGYSEIFLMPLWTSYTVSKQAEVSSVPDHLTSCVRPDVRVSPSFSQNCLA |
| | YKNDKQMSYGFLFPPYLSSSPEAKYDAFLVTNMVPMYPAFKRVWNYFQRVLVKKYASERNGVNVISG |
| | PIFDYDYDGLHDTEDKIKQYVEGSSIPVPTHYYSIITSCLDFTQPADKCDGPLSVSSFILPHRPDNE |
| | ESCNSSEDESKWVEELMKMHTARVRDIEHLTSLDFFRKTSRSYPEILTLKTYLHTYGARF |

| | |
|---|---|
| SEQ ID NO: 285 | 2644 bp |

| | |
|---|---|
| NOV29f, | CCACCATGGCAAGGAGGAGCTCGTTCCAGTCGTGTCAGATAATATCCCTGTTCACTTTTGCCGTTGG |
| CG93541-06 | |
| DNA Sequence | AGTCAATATCTGCTTAGGATTCACTGCACATCGAATTAAGAGAGCAGAAGGATGGGAGGAAGGTCCT |
| | CCTACAGTGCTATCAGACTCCCCCTGGACCAACATCTCCGGATCTTGCAAGGGCAGGTGCTTTGAAC |
| | TTCAAGAGGCTGGACCTCCTGATTGTCGCTGTGACAACTTGTGTAAGAGCTATACCAGTTGCTGCCA |
| | TGACTTTGATGAGCTGTGTTTGAAGACAGCCCGTGGCTGGGAGTGTACTAAGGACAGATGTGGAGAA |
| | GTCAGAAATGAAGAAAATGCCTGTCACTGCTCAGAGGACTGCTTGGCCAGGGGAGACTGCTGTACCA |
| | ATTACCAAGTGGTTTGCAAAGGAGAGTCGCATTGGGTTGATGATGACTGTGAGGAAATAAAGGCCGC |
| | AGAATGCCCTGCAGGGTTTGTTCGCCCTCCATTAATCATCTTCTCCGTGGATGGCTTCCGTGCATCA |
| | TACATGAAGAAAGGCAGCAAAGTCATGCCTAATATTGAAAAACTAAGGTCTTGTGGCACACACTCTC |
| | CCTACATGAGGCCGGTGTACCCAACTAAAACCTTTCCTAACTTATACACTTTGGCCACTGGGCTATA |
| | TCCAGAATCACATGGAATTGTTGGCAATTCAATGTATGATCCTGTATTTGATGCCACTTTTCATCTG |
| | CGAGGGCGAGAGAAATTTAATCATAGATGGTGGGGAGGTCAACCGCTATGGATTACAGCCACCAAGC |
| | AAGGGGTGAAAGCTGGAACATTCTTTTGGTCTGTTGTCATCCCTCACGAGCGGAGAATATTAACCAT |
| | ATTGCAGTGGCTCACCCTGCCAGATCATGAGAGGCCTTCGGTCTATGCCTTCTATTCTGAGCAACCT |
| | GATTTCTCTGGACACAAATATGGCCCTTTCGGCCCTGAGATGACAAATCCTCTGAGGGAAATCGACA |
| | AAATTGTGGGGCAATTAATGGATGGACTGAAACAACTAAAACTGCATCGGTGTGTCAACGTCATCTT |
| | TGTCGGAGACCATGGAATGGAAGATGTCACATGTGATAGAACTGAGTTCTTGAGTAATTACCTAACT |
| | AATGTGGATGATATTACTTTAGTGCCTGGAACTCTAGGAAGAATTCGATCCAAATTTAGCAACAATG |
| | CTAAATATGACCCCAAAGCCATTATTGCCAATCTCACGTGTAAAAAACCAGATCAGCACTTTAAGCC |
| | TTACTTGAAACAGCACCTTCCCAAACGTTTGCACTATGCCAACAACAGAAGAATTGAGGATATCCAT |
| | TTATTGGTGGAACGCAGATGGCATGTTGCAAGGAAACCTTTGGATGTTTATAAGAAACCATCAGGAA |
| | AATGCTTTTTCCAGGGAGACCACGGATTTGATAACAAGGTCAACAGCATGCAGACTGTTTTTGTAGG |
| | TTATGGCCCAACATTTAAGTACAAGACTAAAGTGCCTCCATTTGAAAACATTGAACTTTACAATGTT |
| | ATGTGTGATCTCCTGGGATTGAAGCCAGCTCCTAATAATGGGACCCACGGAAGTTTGAATCATCTCC |
| | TGCGCACTAATACCTTCAGGCCAACCATGCCAGAGGAAGTTACCAGACCCAATTATCCAGGGATTAT |
| | GTACCTTCAGTCTGATTTTGACCTGGGCTGCACTTGTGATGATAAGGTAGAGCCAAAGAACAAGTTG |
| | GATGAACTCAACAAACGGCTTCATACAAAAGGGTCTACAGAAGAGAGACACCTCCTCTATGGGCGAC |
| | CTGCAGTGCTTTATCGGACTAGATATGATATCTTATATCACACTGACTTTGAAAGTGGTTATAGTGA |
| | AATATTCCTAATGCCACTCTGGACATCATATACTGTTTCCAAACAGGCTGAGGTTTCCAGCGTTCCT |
| | GACCATCTGACCAGTTGCGTCCGGCCTGATGTCCGTGTTTCTCCGAGTTTCAGTCAGAACTGTTTGG |
| | CCTACAAAAATGATAAGCAGATGTCCTACGGATTCCTCTTTCCTCCTTATCTGAGCTCTTCACCAGA |
| | GGCTAAATATGATGCATTCCTTGTAACCAATATGGTTCCAATGTATCCTGCTTTCAAACGGGTCTGG |
| | AATTATTTCCAAAGGGTATTGGTGAAGAAATATGCTTCGGAAAGAAATGGAGTTAACGTGATAAGTG |
| | GACCAATCTTCGACTATGACTATGATGGCTTACATGACACAGAAGACAAAATAAAACAGTACGTGGA |
| | AGGCAGTTCCATTCCTGTTCCAACTCACTACTACAGCATCATCACCAGCTGTCTGGATTTTACTCAG |
| | CCTGCCGACAAGTGTGACGGCCCTCTCTCTGTGTCCTCCTTCATCCTGCCTCACCGGCCTGACAACG |
| | AGGAGAGCTGCAATAGCTCAGAGGACGAATCAAAATGGGTAGAAGAACTCATGAAGATGCACACAGC |
| | TAGGGTGCGTGACATTGAACATCTCACCAGCCTGGACTTCTTCCGAAAGACCAGCCGCAGCTACCCA |
| | GAAATCCTGACACTCAAGACATACTTGCATACATATGAGAGCGAGATTCACCATCACCACCATCACT |
| | AAGCGGCGTCGAGTCTAGAGGGCCGTTTAAC |

| | | |
|---|---|---|
| ORF Start: at 3 | | ORF Stop: TAA at 2613 |
| SEQ ID NO: 286 | 870 aa | MW at 99926.5 kD |

| | |
|---|---|
| NOV29f, | TMARRSSFQSCQIISLFTFAVGVNICLGFTAHRIKRAEGWEEGPPTVLSDSPWTNISGSCKGRCFEL |
| CG93541-06 | |
| Protein | QEAGPPDCRCDNLCKSYTSCCHDFDELCLKTARGWECTKDRCGEVRNEENACHCSEDCLARGDCCTN |
| Sequence | |
| | YQVVCKGESHWVDDDCEEIKAAECPAGFVRPPLIIFSVDGFRASYMKKGSKVMPNIEKLRSCGTHSP |
| | YMRPVYPTKTFPNLYTLATGLYPESHGIVGNSMYDPVFDATFHLRGREKFNHRWWGGQPLWITATKQ |
| | GVKAGTFFWSVVIPHERRILTILQWLTLPDHERPSVYAFYSEQPDFSGHKYGPFGPEMTNPLREIDK |
| | IVGQLMDGLKQLKLHRCVNVIFVGDHGMEDVTCDRTEFLSNYLTNVDDITLVPGTLGRIRSKFSNNA |
| | KYDPKAIIANLTCKKPDQHFKPYLKQHLPKRLHYANNRRIEDIHLLVERRWHVARKPLDVYKKPSGK |
| | CFFQGDHGFDNKVNSMQTVFVGYGPTFKYKTKVPPFENIELYNVMCDLLGLKPAPNNGTHGSLNHLL |
| | RTNTFRPTMPEEVTRPNYPGIMYLQSDFDLGCTCDDKVEPKNKLDELNKRLHTKGSTEERHLLYGRP |
| | AVLYRTRYDILYHTDFESGYSEIFLMPLWTSYTVSKQAEVSSVPDHLTSCVRPDVRVSPSFSQNCLA |
| | YKNDKQMSYGFLFPPYLSSSPEAKYDAFLVTNMVPMYPAFKRVWNYFQRVLVKKYASERNGVNVISG |
| | PIFDYDYDGLHDTEDKIKQYVEGSSIPVPTHYYSIITSCLDFTQPADKCDGPLSVSSFILPHRPDNE |
| | ESCNSSEDESKWVEELMKMHTARVRDIEHLTSLDFFRKTSRSYPEILTLKTYLHTYESEIHHHHHH |

Sequence comparison of the above protein sequences yields the following sequence relationships shown in Table 29B.

TABLE 29B
Comparison of NOV29a against NOV29b through NOV29f.

| | NOV29a Residues/ | Identities/Similarities |
|---|---|---|
| Protein Sequence | Match Residues | for the Matched Region |
| NOV29b | 10 . . . 871 | 861/862 (99%) |
| | 2 . . . 863 | 861/862 (99%) |
| NOV29c | 10 . . . 202 | 179/193 (92%) |
| | 2 . . . 189 | 182/193 (93%) |
| NOV29d | 53 . . . 313 | 257/261 (98%) |
| | 18 . . . 278 | 259/261 (98%) |
| NOV29e | 10 . . . 867 | 858/858 (100%) |
| | 3 . . . 860 | 858/858 (100%) |
| NOV29f | 10 . . . 871 | 862/862 (100%) |
| | 3 . . . 864 | 862/862 (100%) |

Further analysis of the NOV29a protein yielded the following properties shown in Table 29C.

TABLE 29C
Protein Sequence Properties NOV29a

| | |
|---|---|
| SignalP analysis: | Cleavage site between residues 36 and 37 |
| PSORT II analysis: | PSG: a new signal peptide prediction method |
| | N-region: length 11; pos.chg 1; neg.chg 0 |
| | H-region: length 0; peak value −6.81 |
| | PSG score: −11.21 |
| | GvH: von Heijne's method for signal seq. recognition |
| | GvH score (threshold: −2.1): −4.77 |
| | possible cleavage site: between 35 and 36 |
| | >>> Seems to have no N-terminal signal peptide |
| | ALOM: Klein et al's method for TM region allocation |
| | Init position for calculation: 1 |
| | Tentative number of TMS(s) for the threshold 0.5: 1 |
| | Number of TMS(s) for threshold 0.5: 1 |
| | INTEGRAL Likelihood = −5.04 Transmembrane 20-36 |
| | PERIPHERAL Likelihood = 4.24 (at 163) |
| | ALOM score: −5.04 (number of TMSs: 1) |
| | MTOP: Prediction of membrane topology (Hartmann et al.) |
| | Center position for calculation: 27 |
| | Charge difference: −5.5 C(0.5) − N(6.0) |
| | N >= C: N-terminal side will be inside |
| | >>> membrane topology: type 2 (cytoplasmic tail 1 to 20) |
| | MITDISC: discrimination of mitochondrial targeting seq |

| | | | |
|---|---|---|---|
| R content: | 4 | Hyd Moment(75): | 2.28 |
| Hyd Moment(95): | 3.41 | G content: | 3 |
| D/E content: | 1 | S/T content: | 7 |
| Score: −1.51 | | | |

```
Gavel: prediction of cleavage sites for mitochondrial preseq
R-2 motif at 53 KRA|EG
NUCDISC: discrimination of nuclear localization signals
pat4: none
pat7: none
bipartite: none
content of basic residues: 11.9%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
34.8%: mitochondrial
26.1%: cytoplasmic
17.4%: Golgi
  4.3%: vacuolar
  4.3%: extracellular, including cell wall
  4.3%: nuclear
  4.3%: vesicles of secretory system
  4.3%: endoplasmic reticulum
>> prediction for CG93541-05 is mit (k = 23)
```

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAY71988 | Human teratocarcinoma | 10 . . . 871 | 861/862 (99%) | 0.0 |
| | autotaxin - Homo sapiens, | 2 . . . 863 | 861/862 (99%) | |
| | 863 aa. [WO200068386-A1, | | | |
| | 16-NOV-2000] | | | |
| AAY71991 | Human autotaxin protein - | 19 . . . 871 | 852/853 (99%) | 0.0 |
| | Homo sapiens, 859 aa. | 7 . . . 859 | 853/853 (99%) | |
| | [WO200068386-A1, | | | |
| | 16-NOV-2000] | | | |
| ABG32516 | Rat lysophospholipase D - | 10 . . . 871 | 810/862 (93%) | 0.0 |
| | Rattus norvegicus, 862 aa. | 2 . . . 862 | 839/862 (96%) | |
| | [WO200253569-A1, | | | |
| | 11-JUL-2002] | | | |
| AAY71999 | Rat autotaxin variant (S289T) - | 19 . . . 871 | 807/853 (94%) | 0.0 |
| | Rattus sp, 858 aa. | 7 . . . 858 | 835/853 (97%) | |
| | [WO200068386-A1, | | | |
| | 16-NOV-2000] | | | |
| AAY71997 | Rat autotaxin variant (S236T) - | 19 . . . 871 | 807/853 (94%) | 0.0 |
| | Rattus sp, 858 aa. | 7 . . . 858 | 835/853 (97%) | |
| | [WO200068386-A1, | | | |
| | 16-NOV-2000] | | | |

In a BLAST search of public sequence datbases, the NOV29a protein was found to have homology to the proteins shown in the BLASTP data in Table 29E.

TABLE 29E
Public BLASTP Results for NOV29a

| | | NOV29a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q13822 | Ectonucleotide | 10 . . . 871 | 862/862 (100%) | 0.0 |
| | pyrophosphatase/ | 2 . . . 863 | 862/862 (100%) | |
| | phosphodiesterase 2 (E-NPP | | | |
| | 2) (Phosphodiesterase I/ | | | |
| | nucleotide pyrophosphatase | | | |
| | 2) (Phosphodiesterase I | | | |
| | alpha) (PD-Ialpha) | | | |
| | (Autotaxin) [Includes: | | | |
| | Alkaline phosphodiesterase I | | | |
| | (EC 3.1.4.1); Nucleotide | | | |
| | pyrophosphatase (EC 3.6.1.9) | | | |
| | (NPPase)] - Homo sapiens | | | |
| | (Human), 863 aa. | | | |
| CAC18956 | Sequence 8 from Patent | 19 . . . 871 | 852/853 (99%) | 0.0 |
| | WO0068386 - Homo sapiens | 7 . . . 859 | 853/853 (99%) | |
| | (Human), 859 aa. | | | |
| Q9R1E6 | Ectonucleotide | 10 . . . 871 | 813/862 (94%) | 0.0 |
| | pyrophosphatase/ | 2 . . . 862 | 839/862 (97%) | |
| | phosphodiesterase 2 (E-NPP | | | |
| | 2) (Phosphodiesterase I/ | | | |
| | nucleotide pyrophosphatase | | | |
| | 2) (Phosphodiesterase I | | | |
| | alpha) (PD-Ialpha) [Includes: | | | |
| | Alkaline phosphodiesterase I | | | |
| | (EC 3.1.4.1); Nucleotide | | | |
| | pyrophosphatase (EC 3.6.1.9) | | | |
| | (NPPase)] - Mus musculus | | | |
| | Mouse), 862 aa. | | | |
| CAC18955 | Sequence 1 from Patent | 19 . . . 871 | 806/853 (94%) | 0.0 |
| | WO0068386 - Rattus sp, 858 | 7 . . . 858 | 835/853 (97%) | |
| Q64610 | Ectonucleotide | 10 . . . 871 | 794/887 (89%) | 0.0 |
| | pyrophosphatase/phosphodies | 2 . . . 885 | 825/887 (92%) | |
| | terase 2 (E-NPP 2) | | | |
| | (Phosphodiesterase | | | |
| | I/nucleotide pyrophosphatase | | | |
| | 2) (Phosphodiesterase I | | | |
| | alpha) (PD-Ialpha) [Includes: | | | |
| | Alkaline phosphodiesterase I | | | |
| | (EC 3.1.4.1); Nucleotide | | | |
| | pyrophosphatase (EC 3.6.1.9) | | | |
| | (NPPase)] - Rattus | | | |
| | norvegicus (Rat), 885 aa. | | | |

PFam analysis predicts that the NOV29a protein contains the domains shown in the Table 29F.

TABLE 29F
Domain Analysis of NOV29a

| | | Identities/ | |
|---|---|---|---|
| | NOV29a Match | Similarities for | Expect |
| Pfam Domain | Region | the Matched Region | value |
| Somatomedin_B | 63 . . . 107 | 23/47 (49%) | 7.4e−19 |
| | | 40/47 (85%) | |
| Somatomedin_B | 108 . . . 151 | 21/47 (45%) | 7.6e−17 |
| | | 41/47 (87%) | |
| Phosphodiest | 153 . . . 510 | 177/416 (43%) | 8.4e−199 |
| | | 353/416 (85%) | |

### Example 30

The NOV30 clone was analyzed, and the nucleotide and encoded polypeptide sequences are shown in Table 30A.

TABLE 30A
NOV30 Sequence Analysis

| | |
|---|---|
| SEQ ID NO: 287 | 904 bp |

| | |
|---|---|
| NOV30a, | CACCGGATCCACTTCCGGGAACGCCGGGGAACCGCAGTAGCCGCCTGCTAGTGGCGCTGCTAGCCGG |
| CG93735-05 | |
| DNA Sequence | CCGGCGCAGGCTGCCGAGCGGGTGAGCGCGCAGGCCAGGCCAAAGCCCTGGTACCCGCGCGGTGCGG |
| | GCCTCAGTCTGCGGCCATGGGGGCGTCCGCGCGGCTGCTGCGAGCGGTGATCATGGGGGCCCCGGGC |
| | TCGGGCAAGGGCACCGTGTCGTCGCGCATCACTACACACTTCGAGCTGAAGCACCTCTCCAGCGGGG |
| | ACCTGCTCCGGGACAACATGCTGCGGGGCACAGAAATTGGCGTGTTAGCCAAGGCTTTCATTGACCA |
| | AGGGAAACTCATCCCAGATGATGTCATGACTCGGCTGGCCCTTCATGAGCTGAAAAATCTCACCCAG |
| | TATAGCTGGCTGTTGGATGGTTTTCCAAGGACACTTCCACAGGCAGAAGCCCTAGATAGAGCTTATC |
| | AGATCGACACAGTGATTAACCTGAATGTGCCCTTTGAGGTCATTAAACAACGCCTTACTGCTCGCTG |
| | GATTCATCCCGCCAGTGGCCGAGTCTATAACATTGAATTCAACCCTCCCAAAACTGTGGGCATTGAT |
| | GACCTGACTGGGGAGCCTCTCATTCAGCGTGAGGATGATAAACCAGAGACGGTTATCAAGAGACTAA |
| | AGGCTTATGAAGACCAAACAAAGCCAGTCCTGGAATATTACCAGAAAAAAGGGGTGCTGGAAACATT |
| | CTCCGGAACAGAAACCAACAAGATTTGGCCCTATGTATATGCTTTCCTACAAACTAAAGTTCCACAA |
| | AGAAGCCAGAAAGCTTCAGTTACTCCATGAGGAGAAATGTGTGTAACTATTAATAGTAAGATGGGCA |
| | AACCTCCTAGTCCTTGCATTTAGGTCGACGCGT |

| | | |
|---|---|---|
| ORF Start: at 64 | | ORF Stop: TGA at 832 |
| SEQ ID NO: 288 | 256 aa | MW at 28269.2 kD |

| | |
|---|---|
| NOV30a, | PAGAGCRAGERAGQAKALVPARCGPQSAAMGASARLLRAVIMGAPGSGKGTVSSRITTHFELKHLSS |
| CG93735-05 | |
| Protein Sequence | GDLLRDNMLRGTEIGVLAKAFIDQGKLIPDDVMTRLALHELKNLTQYSWLLDGFPRTLPQAEALDRA |
| | YQIDTVINLNVPFEVIKQRLTARWIHPASGRVYNIEFNPPKTVGIDDLTGEPLIQREDDKPETVIKR |
| | LKAYEDQTKPVLEYYQKKGVLETFSGTETNKIWPYVYAFLQTKVPQRSQKASVTP |

| | |
|---|---|
| SEQ ID NO: 289 | 1021 bp |

| | |
|---|---|
| NOV30b, | ACTTCCGGGAACGCCGGGGAACCGCAGTAGCCGCCTGCTAGTGGCGCTGCTAGCCGGCCGGCGCAGG |
| CG93735-01 | |
| DNA Sequence | CTGCCGAGCGGGTGAGCGCGCAGGCCAGGCCAAAGCCCTGGTACCCGCGCGGTGCGGGCCTCAGTCT |
| | GCGGCCATGGGGGCGTCGGGGCGGCTGCTGCGAGCGGTGATCATGGGGGCCCCGGGCTCGGGCAAGG |
| | GCACCGTGTCGTCCCGCATCACTACACACTTCGAGCTGAAGCACCTCTCCCGCGGGGACCTGCTCCG |
| | GGACAACATGCTGCGGGGCACAGAAATTGGCGTGTTAGCCCAGGCTTTCATTGACCAAGGGAAACTC |
| | ATCCCAGATTATGTCACGACTCGGCTGGCCCTTCATGAGCTGAAAAACCTCACCCAGTATAGCTGGC |
| | TGTTGGATGGTTTTCCAAGGACACTTCCACAGGCAGAAGCCCTAGATAGAGCTTATCAGATCGACAC |
| | AGTGATTAACCTGAATGTGCCCTTTGAGGTCATTAAACAACGCCTTACTGCTCGCTGGATTCATCCC |
| | GCCAGTGGCCGAGTCTATAACATTGAATTCAACCCTCCCAAAACTGTGGGCATTGATGACCTGACTG |
| | GGGAGCCTCTCATTCAGCGTGAGGATGATAAACCAGAGACGGTTATCAAGAGACTAAAGGCTTATGA |
| | AGACCAAACAAAGCCAGTCCTGGAATATTACCAGAAAAAAGGGGTGTTGGAAACATTCTCCGGAACA |
| | GAAACCAACAAGATTTGGCCCTATGTATATGCTTTCCTACAAACTAAAGTTCCACAAAGAAGCCAGA |
| | AAGCTTCAGTTACTCCATGAGGAGAAATGTGTGTAACTATTAATAGTAAGATGGGCAAACCTCCTAG |
| | TCCTTGCATTTAGAAGCTGCTTTTCCTAAGACTTCTAGTATGTATGAATTCTTTGAAAATTATATTA |
| | CTTTTATTTCTACTGATTTTATTTTGGATACTAAGGATGTGCCAAATGATTCGGATACTAAGATGCA |
| | TCGTTTGAAATCATCT |

| | | |
|---|---|---|
| ORF Start: ATG at 141 | | ORF Stop: TGA at 822 |
| SEQ ID NO: 290 | 227 aa | MW at 25638.2 kD |

| | |
|---|---|
| NOV30b, | MGASGRLLRAVIMGAPGSGKGTVSSRITTHFELKHLSRGDLLRDNMLRGTEIGVLAQAFIDQGKLIP |
| CG93735-01 | |
| Protein Sequence | DYVTTRLALHELKNLTQYSWLLDGFPRTLPQAEALDRAYQIDTVINLNVPFEVIKQRLTARWIHPAS |
| | GRVYNIEFNPPKTVGIDDLTGEPLIQREDDKPETVIKRLKAYEDQTKPVLEYYQKKGVLETFSGTET |
| | NKIWPYVYAFLQTKVPQRSQKASVTP |

| | |
|---|---|
| SEQ ID NO: 291 | 707 bp |

| | |
|---|---|
| NOV30c, | CCACCATGGGGGCGTCGGGGCGGCTGCTGCGAGCGGTGATCATGGGGGCCCCGGGCTCGGGCAAGGG |
| 171094650 DNA | |
| Sequence | CACCGTGTCGTCGCGCATCACTACACACTTCGAGCTGAAGCACCTCTCCAGCGGGGACCTGCTCCGG |
| | GACAACATGCTGCGGGGCACAGAAATTGGCGTGTTAGCCAAGGCTTTCATTGACCAAGGGAAACTCA |
| | TCCCAGATGATGTCATGACTCGGCTGGCCCTTCATGAGCTGAAAAATCTCACCCAGTATAGCTGGCT |
| | GTTGGATGGTTTTCCAAGGACACTTCCACAGGCAGAAGCCCTAGATAGAGCTTATCAGATCGACACA |
| | GTGATTAACCTGAATGTGCCCTTTGAGGTCATTAAACAACGCCTTACTGCTCGCTGGATTCATCCCG |
| | CCAGTGGCCGAGTCTATAACATTGAATTCAACCCTCCCAAAACTGTGGGCATTGATGACCTGACTGG |
| | GGAGCCTCTCATTCAGCGTGAGGATGATAAACCAGAGACGGTTATCAAGAGACTAAAGGCTTATGAA |
| | GACCAAACAAAGCCAGTCCTGGAATATTACCAGAAAAAAGGGGTGCTGGAAACATTCTCCGGAACAG |
| | AAACCAACAAGATTTGGCCCTATGTATATGCTTTCCTACAAACTAAAGTTCCACAAAGAAGCCAGAA |
| | AGCTTCAGTTACTCCACACCATCACCACCATCACTGA |

| | | |
|---|---|---|
| ORF Start: at 3 | | ORF Stop: TGA at 705 |
| SEQ ID NO: 292 | 234 aa | MW at 26475.1 kD |

| | |
|---|---|
| NOV30c, | TMGASGRLLRAVIMGAPGSGKGTVSSRITTHFELKHLSSGDLLRDNMLRGTEIGVLAKAFIDQGKLI |
| 171094650 | |
| Protein Sequence | PDDVMTRLALHELKNLTQYSWLLDGFPRTLPQAEALDRAYQIDTVINLNVPFEVIKQRLTARWIHPA |
| | SGRVYNIEFNPPKTVGIDDLTGEPLIQREDDKPETVIKRLKAYEDQTKPVLEYYQKKGVLETFSGTE |
| | TNKIWPYVYAFLQTKVPQRSQKASVTPHHHHHH |

| | |
|---|---|
| SEQ ID NO: 293 | 706 bp |

| | |
|---|---|
| NOV30d, | CACCATGGGGGCGTCGGGGCGGCTGCTGCGAGCGGTGATCATGGGGGCCCCGGGCTCGGGCAAGGGC |
| 173172155 DNA | |
| Sequence | ACCGTGTCGTCGCGCATCACTACACACTTCGAGCTGAAGCACCTCTCCAGCGGGGACCTGCTCCGGG |
| | ACAACATGCTGCGGGGCACAGAAATTGGCGTGTTAGCCAAGGCTTTCATTGACCAAGGGAAACTCAT |
| | CCCAGATGATGTCATGACTCGGCTGGCCCTTCATGAGCTGAAAAATCTCACCCAGTATAGCTGGCTG |
| | TTGGATGGTTTTCCAAGGACACTTCCACAGGCAGAAGCCCTAGATAGAGCTTATCAGATCGACACAG |
| | TGATTAACCTGAATGTGCCCTTTGAGGTCATTAAACAACGCCTTACTGCTCGCTGGATTCATCCCGC |
| | CAGTGGCCGAGTCTATAACATTGAATTCAACCCTCCCAAAACTGTGGGCATTGATGACCTGACTGGG |
| | GAGCCTCTCATTCAGCGTGAGGATGATAAACCAGAGACGGTTATCAAGAGACTAAAGGCTTATGAAG |
| | ACCAAACAAAGCCAGTCCTGGAATATTACCAGAAAAAAGGGGTGCTGGAAACATTCTCCGGAACAGA |
| | AACCAACAAGATTTGGCCCTATGTATATGCTTTCCTACAAACTAAAGTTCCACAAAGAAGCCAGAAA |
| | GCTTCAGTTACTCCACACCATCACCACCATCACTGA |

| | | |
|---|---|---|
| ORF Start: at 2 | | ORF Stop: TGA at 704 |
| SEQ ID NO: 294 | 234 aa | MW at 26475.1 kD |

| | |
|---|---|
| NOV30d, | TMGASGRLLRAVIMGAPGSGKGTVSSRITTHFELKHLSSGDLLRDNMLRGTEIGVLAKAFIDQGKLI |
| 173172155 | PDDVMTRLALHELKNLTQYSWLLDGFPRTLPQAEALDRAYQIDTVINLNVPFEVIKQRLTARWIHPA |
| Protein Sequence | |
| | SGRVYNIEFNPPKTVGIDDLTGEPLIQREDDKPETVIKRLKAYEDQTKPVLEYYQKKGVLETFSGTE |
| | TNKIWPYVYAFLQTKVPQRSQKASVTPHHHHHH |

| | |
|---|---|
| SEQ ID NO: 295 | 730 bp |

| | |
|---|---|
| NOV30e, | GGCGTCGGGGCGGCTGCTGCGAGCGGTGATCATGGGGGCCCCGGGCTCGGGCAAGGGCACCGTGTCG |
| 195803542 DNA | |
| Sequence | TCGCGCATCACTACACACTTCGAGCTGAAGCACCTCTCCAGCGGGGACCTGCTCCGGGACAACATGC |
| | TGCGGGGCACAGAAATTGGCGTGTTAGCCAAGGCTTTCATTGACCAAGGGAAACTCATCCCAGATGA |
| | TGTCATGACTCGGCTGGCCCTTCATGAGCTGAAAAATCTCACCCAGTATAGCTGGCTGTTGGATGGT |
| | TTTCCAAGGACACTTCCACAGGCAGAAGCCCTAGATAGAGCTTATCAGATCGACACAGTGATTAACC |
| | TGAATGTGCCCTTTGAGGTCATTAAACAACGCCTTACTGCTCGCTGGATTCATCCCGCCAGTGGCCG |
| | AGTCTATAACATTGAATTCAACCCTCCCAAAACTGTGGGCATTGATGACCTGACTGGGGAGCCTCTC |
| | ATTCAGCGTGAGGATGATAAACCAGAGACGGTTATCAAGAGACTAAAGGCTTATGAAGACCAAACAA |
| | AGCCAGTCCTGGAATATTACCAGAAAAAAGGGGTGCTGGAAACATTCTCCGGAACAGAAACCAACAA |
| | GATTTGGCCCTATGTATATGCTTTCCTACAAACTAAAGTTCCACAAAGAAGCCAGAAAGCTTCAGTT |
| | ACTCCACACCATCACCACCATCACTGAGCGGCCGCACTCGAGCACCACCACCACCACCAC |

| | | |
|---|---|---|
| ORF Start: at 2 | | ORF Stop: TGA at 695 |
| SEQ ID NO: 296 | 231 aa | MW at 26185.7 kD |

| | |
|---|---|
| NOV30e, | ASGRLLRAVIMGAPGSGKGTVSSRITTHFELKHLSSGDLLRDNMLRGTEIGVLAKAFIDQGKLIPDD |
| 195803542 | |
| Protein Sequence | VMTRLALHELKNLTQYSWLLDGFPRTLPQAEALDRAYQIDTVINLNVPFEVIKQRLTARWIHPASGR |
| | VYNIEFNPPKTVGIDDLTGEPLIQREDDKPETVIKRLKAYEDQTKPVLEYYQKKGVLETFSGTETNK |
| | IWPYVYAFLQTKVPQRSQKASVTPHHHHHH |

| | |
|---|---|
| SEQ ID NO: 297 | 688 bp |

| | |
|---|---|
| NOV30f, | CACCATGGGGGCGTCGGGGCGGCTGCTGCGAGCGGTGATCATGGGGGCCCCGGGCTCGGGCAAGGGC |
| 171093359 DNA | |
| Sequence | ACCGTGTCGTCGCGCATCACTACACACTTCGAGCTGAAGCACCTCTCCAGCGGGGACCTGCTCCGGG |
| | ACAACATGCTGCGGGGCACAGAAATTGGCGTGTTAGCCAAGGCTTTCATTGACCAAGGGAAACTCAT |
| | CCCAGATGATGTCATGACTCGGCTGGCCCTTCATGAGCTGAAAAATCTCACCCAGTATAGCTGGCTG |
| | TTGGATGGTTTTCCAAGGACACTTCCACAGGCAGAAGCCCTAGATAGAGCTTATCAGATCGACACAG |
| | TGATTAACCTGAATGTGCCCTTTGAGGTCATTAAACAACGCCTTACTGCTCGCTGGATTCATCCCGC |
| | CAGTGGCCGAGTCTATAACATTGAATTCAACCCTCCCAAAACTGTGGGCATTGATGACCTGACTGGG |
| | GAGCCTCTCATTCAGCGTGAGGATGATAAACCAGAGACGGTTATCAAGAGACTAAAGGCTTATGAAG |
| | ACCAAACAAAGCCAGTCCTGGAATATTACCAGAAAAAAGGGGTGCTGGAAACATTCTCCGGAACAGA |
| | AACCAACAAGATTTGGCCCTATGTATATGCTTTCCTACAAACTAAAGTTCCACAAAGAAGCCAGAAA |
| | GCTTCAGTTACTCCATGA |

| | | |
|---|---|---|
| ORF Start: at 2 | | ORF Stop: TGA at 686 |
| SEQ ID NO: 298 | 228 aa | MW at 25652.2 kD |

| | |
|---|---|
| NOV30f, | TMGASGRLLRAVIMGAPGSGKGTVSSRITTHFELKHLSSGDLLRDNMLRGTEIGVLAKAFIDQGKLI |
| 171093359 | |
| Protein | PDDVMTRLALHELKNLTQYSWLLDGFPRTLPQAEALDRAYQIDTVINLNVPFEVIKQRLTARWIHPA |
| Sequence | |
| | SGRVYNIEFNPPKTVGIDDLTGEPLIQREDDKPETVIKRLKAYEDQTKPVLEYYQKKGVLETFSGTE |
| | TNKIWPYVYAFLQTKVPQRSQKASVTP |

| | |
|---|---|
| SEQ ID NO: 299 | 688 bp |

| | |
|---|---|
| NOV30g, | CACCATGGGGGCGTCGGGGCGGCTGCTGCGAGCGGTGATCATGGGGGCCCCGGGCTCGGGCAAGGGC |
| 171065502 DNA | |
| Sequence | ACCGTGTCGTCGCGCATCACTACACACTTCGAGCTGAAGCACCTCTCCAGCGGGGACCTGCTCCGGG |
| | ACAACATGCTGCGGGGCACAGAAATTGGCGTGTTAGCCAAGGCTTTCATTGACCAAGGGAAACTCAT |
| | CCCAGATGATGTCATGACTCGGCTGGCCCTTCATGAGCTGAAAAATCTCACCCAGTATAGCTGGCTG |
| | TTGGATGGTTTTCCAAGGACACTTCCACAGGCAGAAGCCCTAGATAGAGCTTATCAGATCGACACAG |
| | TGATTAACCTGAATGTGCCCTTTGAGGTCATTAAACAACGCCTTACTGCTCGCTGGATTCATCCCGC |
| | CAGTGGCCGAGTCTATAACATTGAATTCAACCCTCCCAAAACTGTGGGCATTGATGACCTGACTGGG |
| | GAGCCTCTCATTCAGCGTGAGGATGATAAACCAGAGACGGTTATCAAGAGACTAAAGGCTTATGAAG |
| | ACCAAACAAAGCCAGTCCTGGAATATTACCAGAAAAAAGGGGTGCTGGAAACATTCTCCGGAACAGA |
| | AACCAACAAGATTTGGCCCTATGTATATGCTTTCCTACAAACTAAAGTTCCACAAAGAAGCCAGAAA |
| | GCTTCAGTTACTCCATGA |

| | | |
|---|---|---|
| ORF Start: at 2 | | ORF Stop: TGA at 686 |
| SEQ ID NO: 300 | 228 aa | MW at 25652.2 kD |

| | |
|---|---|
| NOV30g, | TMGASGRLLRAVIMGAPGSGKGTVSSRITTHFELKHLSSGDLLRDNMLRGTEIGVLAKAFIDQGKLI |
| 171065502 | |
| Protein Sequence | PDDVMTRLALHELKNLTQYSWLLDGFPRTLPQAEALDRAYQIDTVINLNVPFEVIKQRLTARWIHPA |
| | SGRVYNIEFNPPKTVGIDDLTGEPLIQREDDKPETVIKRLKAYEDQTKPVLEYYQKKGVLETFSGTE |
| | TNKIWPYVYAFLQTKVPQRSQKASVTP |

| | |
|---|---|
| SEQ ID NO: 301 | 688 bp |

| | |
|---|---|
| NOV30h, | CACCATGGGGGCGTCGGGGCGGCTGCTGCGAGCGGTGATCATGGGGGCCCCGGGCTCGGGCAAGGGC |
| 171093533 DNA | |
| Sequence | ACCGTGTCGTCGCGCATCACTACACACTTCGAGCTGAAGCACCTCTCCAGCGGGGACCTGCTCCGGG |
| | ACAACATGCTGCGGGGCACAGAAATTGGCGTGTTAGCCAAGGCTTTCATTGACCAAGGGAAACTCAT |
| | CCCAGATGATGTCATGACTCGGCTGGCCCTTCATGAGCTGAAAAATCTCACCCAGTATAGCTGGCTG |
| | TTGGATGGTTTTCCAAGGACACTTCCACAGGCAGAAGCCCTAGATAGAGCTTATCAGATCGACACAG |
| | TGATTAACCTGAATGTGCCCTTTGAGGTCATTAAACAACGCCTTACTGCTCGCTGGATTCATCCCGC |
| | CAGTGGCCGAGTCTATAACATTGAATTCAACCCTCCCAAAACTGTGGGCATTGATGACCTGACTGGG |
| | GAGCCTCTCATTCAGCGTGAGGATGATAAACCAGAGACGGTTATCAAGAGACTAAAGGCTTATGAAG |
| | ACCAAACAAAGCCAGTCCTGGAATATTACCAGAAAAAAGGGGTGCTGGAAACATTCTCCGGAACAGA |
| | AACCAACAAGATTTGGCCCTATGTATATGCTTTCCTACAAACTAAAGTTCCACAAAGAAGCCAGAAA |
| | GCTTCAGTTACTCCATGA |

| | | |
|---|---|---|
| ORF Start: at 2 | | ORF Stop: TGA at 686 |
| SEQ ID NO: 302 | 228 aa | MW at 25652.2 kD |

| | |
|---|---|
| NOV30h, | TMGASGRLLRAVIMGAPGSGKGTVSSRITTHFELKHLSSGDLLRDNMLRGTEIGVLAKAFIDQGKLI |
| 171093533 | |
| Protein Sequence | PDDVMTRLALHELKNLTQYSWLLDGFPRTLPQAEALDRAYQIDTVINLNVPFEVIKQRLTARWIHPA |
| | SGRVYNIEFNPPKTVGIDDLTGAPLIQREDDKPETVIKRLKAYEDQTKPVLEYYQKKGVLETFSGTE |
| | TNKIWPYVYAFLQTKVPQRSQKASVTP |

| | |
|---|---|
| SEQ ID NO: 303 | 710 bp |

| | |
|---|---|
| NOV30i, | CCACCATGGGCCACCATCACCACCATCACGGGGCGTCGGGGCGGCTGCTGCGAGCGGTGATCATGGG |
| 171094630 | |
| DNA Sequence | GGCCCCGGGCTCGGGCAAGGGCACCGTGTCGTCGCGCATCACTACACACTTCGAGCTGAAGCACCTC |
| | TCCAGCGGGGACCTGCTCCGGGACAACATGCTGCGGGGCACAGAAATTGGCGTGTTAGCCAAGGCTT |
| | TCATTGACCAAGGGAAACTCATCCCAGATGATGTCATGACTCGGCTGGCCCTTCATGAGCTGAAAAA |
| | TCTCACCCAGTATAGCTGGCTGTTGGATGGTTTTCCAAGGACACTTCCACAGGCAGAAGCCCTAGAT |
| | AGAGCTTATCAGATCGACACAGTGATTAACCTGAATGTGCCCTTTGAGGTCATTAAACAACGCCTTA |
| | CTGCTCGCTGGATTCATCCCGCCAGTGGCCGAGTCTATAACATTGAATTCAACCCTCCCAAAACTGT |
| | GGGCATTGATGACCTGACTGGGGAGCCTCTCATTCAGCGTGAGGATGATAAACCAGAGACGGTTATC |
| | AAGAGACTAAAGGCTTATGAAGACCAAACAAAGCCAGTCCTGGAATATTACCAGAAAAAAGGGGTGC |
| | TGGAAACATTCTCCGGAACAGAAACCAACAAGATTTGGCCCTATGTATATGCTTTCCTACAAACTAA |
| | AGTTCCACAAAGAAGCCAGAAAGCTTCAGTTACTCCATGA |

| | | |
|---|---|---|
| ORF Start: at 3 | | ORF Stop: TGA at 708 |
| SEQ ID NO: 304 | 235 aa | MW at 26532.1 kD |

| | |
|---|---|
| NOV30i, | TMGHHHHHHGASGRLLRAVIMGAPGSGKGTVSSRITTHFELKHLSSGDLLRDNMLRGTEIGVLAKAF |
| 171094630 | |
| Protein | IDQGKLIPDDVMTRLALHELKNLTQYSWLLDGFPRTLPQAEALDRAYQIDTVINLNVPFEVIKQRLT |
| Sequence | |
| | ARWIHPASGRVYNIEFNPPKTVGIDDLTGEPLIQREDDKPETVIKRLKAYEDQTKPVLEYYQKKGVL |
| | ETFSGTETNKIWPYVYAFLQTKVPQRSQKASVTP |

| | |
|---|---|
| SEQ ID NO: 305 | 904 bp |

| | |
|---|---|
| NOV30j, | CACCGGATCCACTTCCGGGAACGCCGGGGAACCGCAGTAGCCGCCTGCTAGTGGCGCTGCTAGCCGG |
| 278391231 | |
| DNA Sequence | CCGGCGCAGGCTGCCGAGCGGGTGAGCGCGCAGGCCAGGCCAAAGCCCTGGTACCCGCGCGGTGCGG |
| | GCCTCAGTCTGCGGCCATGGGGGCGTCCGCGCGGCTGCTGCGAGCGGTGATCATGGGGGCCCCGGGC |
| | TCGGGCAAGGGCACCGTGTCGTCGCGCATCACTACACACTTCGAGCTGAAGCACCTCTCCAGCGGGG |
| | ACCTGCTCCGGGACAACATGCTGCGGGGCACAGAAATTGGCGTGTTAGCCAAGGCTTTCATTGACCA |
| | AGGGAAACTCATCCCAGATGATGTCATGACTCGGCTGGCCCTTCATGAGCTGAAAAATCTCACCCAG |
| | TATAGCTGGCTGTTGGATGGTTTTCCAAGGACACTTCCACAGGCAGAAGCCCTAGATAGAGCTTATC |
| | AGATCGACACAGTGATTAACCTGAATGTGCCCTTTGAGGTCATTAAACAACGCCTTACTGCTCGCTG |
| | GATTCATCCCGCCAGTGGCCGAGTCTATAACATTGAATTCAACCCTCCCAAAACTGTGGGCATTGAT |
| | GACCTGACTGGGGAGCCTCTCATTCAGCGTGAGGATGATAAACCAGAGACGGTTATCAAGAGACTAA |
| | AGGCTTATGAAGACCAAACAAAGCCAGTCCTGGAATATTACCAGAAAAAAGGGGTGCTGGAAACATT |
| | CTCCGGAACAGAAACCAACAAGATTTGGCCCTATGTATATGCTTTCCTACAAACTAAAGTTCCACAA |
| | AGAAGCCAGAAAGCTTCAGTTACTCCATGAGGAGAAATGTGTGTAACTATTAATAGTAAGATGGGCA |
| | AACCTCCTAGTCCTTGCATTTAGGTCGACGCGT |

| | | |
|---|---|---|
| ORF Start: at 64 | | ORF Stop: TGA at 832 |
| SEQ ID NO: 306 | 256 aa | MW at 28269.2 kD |

| | |
|---|---|
| NOV30j, | PAGAGCRAGERAGQAKALVPARCGPQSAAMGASARLLRAVIMGAPGSGKGTVSSRITTHFELKHLSS |
| 278391231 | |
| Protein | GDLLRDNMLRGTEIGVLAKAFIDQGKLIPDDVMTRLALHELKNLTQYSWLLDGFPRTLPQAEALDRA |
| Sequence | |
| | YQIDTVINLNVPFEVIKQRLTARWIHPASGRVYNIEFNPPKTVGIDDLTGEPLIQREDDKPETVIKR |
| | LKAYEDQTKPVLEYYQKKGVLETFSGTETNKIWPYVYAFLQTKVPQRSQKASVTP |

| | |
|---|---|
| SEQ ID NO: 307 | 904 bp |

| | |
|---|---|
| NOV30k, | CACCGGATCCACTTCCGGGAACGCCGGGGAACCGCAGTAGCCGCCTGCTAGTGGCGCTGCTAGCCGG |
| 283291704 DNA | |
| Sequence | CCGGCGCAGGCTGCCGAGCGGGTGAGCGCGCAGGCCAGGCCAAAGCCCTGGTACCCGCGCGGTGCGG |
| | GCCTCAGTCTGCGGCCATGGGGGCGTCCGCGCGGCTGCTGCGAGCGGTGATCATGGGGGCCCCGGGC |
| | TCGGGCAAGGGCACCGTGTCGTCGCGCATCACTACACACTTCGAGCTGAAGCACCTCTCCAGCGGGG |
| | ACCTGCTCCGGGACAACATGCTGCGGGGCACAGAAATTGGCGTGTTAGCCAAGGCTTTCATTGACCA |
| | AGGGAAACTCATCCCAGATGATGTCATGACTCGGCTGGCCCTTCATGAGCTGAAAAATCTCACCCAG |
| | TATAGCTGGCTGTTGGATGGTTTTCCAAGGACACTTCCACAGGCAGAAGCCCTAGATAGAGCTTATC |
| | AGATCGACACAGTGATTAACCTGAATGTGCCCTTTGAGGTCATTAAACAACGCCTTACTGCTCGCTG |
| | GATTCATCCCGCCAGTGGCCGAGTCTATAACATTGAATTCAACCCTCCCAAAACTGTGGGCATTCAT |
| | GACCTGACTGGGGAGCCTCTCATTCAGCGTGAGGATGATAAACCAGAGACGGTTATCAAGAGACTAA |
| | AGGCTTATGAAGACCAAACAAAGCCAGTCCTGGAATATTACCAGAAAAAAGGGGTGCTGGAAACATT |
| | CTCCGGAACAGAAACCAACAAGATTTGGCCCTATGTATATGCTTTCCTACAAACTAAAGTTCCACAA |
| | AGAAGCCAGAAAGCTTCAGTTACTCCATGAGGAGAAATGTGTGTAACTATTAATAGTAAGATGGGCA |
| | AACCTCCTAGTCCTTGCATTTAGTCTAGACTAG |

| | | |
|---|---|---|
| ORF Start: at 64 | | ORF Stop: TGA at 832 |
| SEQ ID NO: 308 | 256 aa | MW at 28269.2 kD |

| | |
|---|---|
| NOV30k, | PAGAGCRAGERAGQAKALVPARCGPQSAAMGASARLLRAVIMGAPGSGKGTVSSRITTHFELKHLSS |
| 283291704 | |
| Protein Sequence | GDLLRDNMLRGTEIGVLAKAFIDQGKLIPDDVMTRLALHELKNLTQYSWLLDGFPRTLPQAEALDRA |
| | YQIDTVINLNVPFEVIKQRLTARWIHPASGRVYNIEFNPPKTVGIDDLTGEPLIQREDDKPETVIKR |
| | LKAYEDQTKPVLEYYQKKGVLETFSGTETNKIWPYVYAFLQTKVPQRSQKASVTP |

| | |
|---|---|
| SEQ ID NO: 309 | 688 bp |

| | |
|---|---|
| NOV30l, | CACCATGGGGGCGTCGGGGCGGCTGCTGCGAGCGGTGATCATGGGGGCCCCGGGCTCGGGCAAGGGC |
| CG93735-02 | |
| DNA Sequence | ACCGTGTCGTCGCGCATCACTACACACTTCGAGCTGAAGCACCTCTCCAGCGGGGACCTGCTCCGGG |
| | ACAACATGCTGCGGGGCACAGAAATTGGCGTGTTAGCCAAGGCTTTCATTGACCAAGGGAAACTCAT |
| | CCCAGATGATGTCATGACTCGGCTGGCCCTTCATGAGCTGAAAAATCTCACCCAGTATAGCTGGCTG |
| | TTGGATGGTTTTCCAAGGACACTTCCACAGGCAGAAGCCCTAGATAGAGCTTATCAGATCGACACAG |
| | TGATTAACCTGAATGTGCCCTTTGAGGTCATTAAACAACGCCTTACTGCTCGCTGGATTCATCCCGC |
| | CAGTGGCCGAGTCTATAACATTGAATTCAACCCTCCCAAAACTGTGGGCATTGATGACCTGACTGGG |
| | GAGCCTCTCATTCAGCGTGAGGATGATAAACCAGAGACGGTTATCAAGAGACTAAAGGCTTATGAAG |
| | ACCAAACAAAGCCAGTCCTGGAATATTACCAGAAAAAAGGGGTGCTGGAAACATTCTCCGGAACAGA |
| | AACCAACAAGATTTGGCCCTATGTATATGCTTTCCTACAAACTAAAGTTCCACAAAGAAGCCAGAAA |
| | GCTTCAGTTACTCCATGA |

| | | |
|---|---|---|
| ORF Start: ATG at 5 | | ORF Stop: TGA at 686 |
| SEQ ID NO: 310 | 227 aa | MW at 25551.1 kD |

| | |
|---|---|
| NOV30l, | MGASGRLLRAVIMGAPGSGKGTVSSRITTHFELKHLSSGDLLRDNMLRGTEIGVLAKAFIDQGKLIP |
| CG93735-02 | |
| Protein | DDVMTRLALHELKNLTQYSWLLDGFPRTLPQAEALDRAYQIDTVINLNVPFEVIKQRLTARWIHPAS |
| Sequence | |
| | GRVYNIEFNPPKTVGIDDLTGEPLIQREDDKPETVIKRLKAYEDQTKPVLEYYQKKGVLETFSGTET |
| | NKIWPYVYAFLQTKVPQRSQKASVTP |

| | |
|---|---|
| SEQ ID NO:311 | 709 bp |

| | |
|---|---|
| NOV30m, | CCACCATGGGCCACCATCACCACCATCACGGGGCGTCGGGGCGGCTGCTGCGAGCGGTGATCATGG |
| CG93735-03 | |
| DNA Sequence | GGGCCCCGGGCTCGGGCAAGGGCACCGTGTCGTCGCGCATCACTACACACTTCGAGCTGAAGCACC |
| | TCTCCAGCGGGGACCTGCTCCGGGACAACATGCTGCGGGGCACAGAAATTGGCGTGTTAGCCAAGG |
| | CTTTCATTGACCAAGGGAAACTCATCCCAGATGATGTCATGACTCGGCTGGCCCTTCATGAGCTGA |
| | AAAATCTCACCCAGTATAGCTGGCTGTTGGATGGTTTTCCAAGGACACTTCCACAGGCAGAAGCCC |
| | TAGATAGAGCTTATCAGATCGACACAGTGATTAACCTGAATGTGCCCTTTGAGGTCATTAAACAAC |
| | GCCTTACTGCTCGCTGGATTCATCCCGCCAGTGGCCGAGTCTATAACATTGAATTCAACCCTCCCA |
| | AAACTGTGGGCATTGATGACCTGACTGGGGAGCCTCTCATTCAGCGTGAGGATGATAAACCAGAGA |
| | CGGTTATCAAGAGACTAAAGGCTTATGAAGACCAAACAAAGCCAGTCCTGGAATATTACCAGAAAA |
| | AAGGGGTGCTGGAAACATTCTCCGGAACAGAAACCAACAAGATTTGGCCCTATGTATATGCTTTCC |
| | TACAAACTAAAGTTCCACAAAGAAGCCAGAAAGCTTCAGTTACTCCATA |

| | | |
|---|---|---|
| ORF Start: at 3 | | ORF Stop: at 708 |
| SEQ ID NO: 312 | 235 aa | MW at 26532.1 kD |

| | |
|---|---|
| NOV30m, | TMGHHHHHHGASGRLLRAVIMGAPGSGKGTVSSRITTHFELKHLSSGDLLRDNMLRGTEIGVLAKA |
| CG93735-03 | |
| Protein Sequence | FIDQGKLIPDDVMTRLALHELKNLTQYSWLLDGFPRTLPQAEALDRAYQIDTVINLNVPFEVIKQR |
| | LTARWIHPASGRVYNIEFNPPKTVGIDDLTGEPLIQREDDKPETVIKRLKAYEDQTKPVLEYYQKK |
| | GVLETFSGTETNKIWPYVYAFLQTKVPQRSQKASVTP |

| | |
|---|---|
| SEQ ID NO: 313 | 707 bp |

| | |
|---|---|
| NOV30n, | CCACCATGGGGGCGTCGGGGCGGCTGCTGCGAGCGGTGATCATGGGGGCCCCGGGCTCGGGCAAGGG |
| CG93735-04 | |
| DNA Sequence | CACCGTGTCGTCGCGCATCACTACACACTTCGAGCTGAAGCACCTCTCCAGCGGGGACCTGCTCCGG |
| | GACAACATGCTGCGGGGCACAGAAATTGGCGTGTTAGCCAAGGCTTTCATTGACCAAGGGAAACTCA |
| | TCCCAGATGATGTCATGACTCGGCTGGCCCTTCATGAGCTGAAAAATCTCACCCAGTATAGCTGGCT |
| | GTTGGATGGTTTTCCAAGGACACTTCCACAGGCAGAAGCCCTAGATAGAGCTTATCAGATCGACACA |
| | GTGATTAACCTGAATGTGCCCTTTGAGGTCATTAAACAACGCCTTACTGCTCGCTGGATTCATCCCG |
| | CCAGTGGCCGAGTCTATAACATTGAATTCAACCCTCCCAAAACTGTGGGCATTGATGACCTGACTGG |
| | GGAGCCTCTCATTCAGCGTGAGGATGATAAACCAGAGACGGTTATCAAGAGACTAAAGGCTTATGAA |
| | GACCAAACAAAGCCAGTCCTGGAATATTACCAGAAAAAAGGGGTGCTGGAAACATTCTCCGGAACAG |
| | AAACCAACAAGATTTGGCCCTATGTATATGCTTTCCTACAAACTAAAGTTCCACAAAGAAGCCAGAA |
| | AGCTTCAGTTACTCCACACCATCACCACCATCACTGA |

| | | |
|---|---|---|
| ORF Start: at 3 | | ORF Stop: TGA at 705 |
| SEQ ID NO: 314 | 234 aa | MW at 26475.1 kD |

| | |
|---|---|
| NOV30n, | TMGASGRLLRAVIMGAPGSGKGTVSSRITTHFELKHLSSGDLLRDNMLRGTEIGVLAKAFIDQGKLI |
| CG93735-04 | |
| Protein Sequence | PDDVMTRLALHELKNLTQYSWLLDGFPRTLPQAEALDRAYQIDTVINLNVPFEVIKQRLTARWIHPA |
| | SGRVYNIEFNPPKTVGIDDLTGEPLIQREDDKPETVIKRLKAYEDQTKPVLEYYQKKGVLETFSGTE |
| | TNKIWPYVYAFLQTKVPQRSQKASVTPHHHHHH |

| | |
|---|---|
| SEQ ID NO: 315 | 904 bp |

| | |
|---|---|
| NOV30o, | CACCGGATCCACTTCCGGGAACGCCGGGGAACCGCAGTAGCCGCCTGCTAGTGGCGCTGCTAGCCGG |
| CG93735-06 | |
| DNA Sequence | CCGGCGCAGGCTGCCGAGCGGGTGAGCGCGCAGGCCAGGCCAAAGCCCTGGTACCCGCGCGGTGCGG |
| | GCCTCAGTCTGCGGCCATGGGGGCGTCCGCGCGGCTGCTGCGAGCGGTGATCATGGGGGCCCCGGGC |
| | TCGGGCAAGGGCACCGTGTCGTCGCGCATCACTACACACTTCGAGCTGAAGCACCTCTCCAGCGGGG |
| | ACCTGCTCCGGGACAACATGCTGCGGGGCACAGAAATTGGCGTGTTAGCCAAGGCTTTCATTGACCA |
| | AGGGAAACTCATCCCAGATGATGTCATGACTCGGCTGGCCCTTCATGAGCTGAAAAATCTCACCCAG |
| | TATAGCTGGCTGTTGGATGGTTTTCCAAGGACACTTCCACAGGCAGAAGCCCTAGATAGAGCTTATC |
| | AGATCGACACAGTGATTAACCTGAATGTGCCCTTTGAGGTCATTAAACAACGCCTTACTGCTCGCTG |
| | GATTCATCCCGCCAGTGGCCGAGTCTATAACATTGAATTCAACCCTCCCAAAACTGTGGGCATTGAT |
| | GACCTGACTGGGGAGCCTCTCATTCAGCGTGAGGATGATAAACCAGAGACGGTTATCAAGAGACTAA |
| | AGGCTTATGAAGACCAAACAAAGCCAGTCCTGGAATATTACCAGAAAAAAGGGGTGCTGGAAACATT |
| | CTCCGGAACAGAAACCAACAAGATTTGGCCCTATGTATATGCTTTCCTACAAACTAAAGTTCCACAA |
| | AGAAGCCAGAAAGCTTCAGTTACTCCATGAGGAGAAATGTGTGTAACTATTAATAGTAAGATGGGCA |
| | AACCTCCTAGTCCTTGCATTTAGTCTAGACTAG |

| | | |
|---|---|---|
| ORF Start: at 64 | | ORF Stop: TGA at 832 |
| SEQ ID NO: 316 | 256 aa | MW at 28269.2 kD |

| | |
|---|---|
| NOV30o, | PAGAGCRAGERAGQAKALVPARCGPQSAAMGASARLLRAVIMGAPGSGKGTVSSRITTHFELKHLSS |
| CG93735-06 | |
| Protein Sequence | GDLLRDNMLRGTEIGVLAKAFIDQGKLIPDDVMTRLALHELKNLTQYSWLLDGFPRTLPQAEALDRA |
| | YQIDTVINLNVPFEVIKQRLTARWIHPASGRVYNIEFNPPKTVGIDDLTGEPLIQREDDKPETVIKR |
| | LKAYEDQTKPVLEYYQKKGVLETFSGTETNKIWPYVYAFLQTKVPQRSQKASVTP |

Sequence comparison of the above protein sequences yields the following sequence relationships shown in Table 30B.

TABLE 30B
Comparison of NOV30a against NOV30b through NOV30o.

| Protein | NOV30a Residues/ | Identities/ |
|---|---|---|
| Sequence | Match Residues | Similarities for the Matched Region |
| NOV30b | 30 . . . 256 | 222/227 (97%) |
| | 1 . . . 227 | 223/227 (97%) |
| NOV30c | 30 . . . 256 | 226/227 (99%) |
| | 2 . . . 228 | 226/227 (99%) |
| NOV30d | 30 . . . 256 | 226/227 (99%) |
| | 2 . . . 228 | 226/227 (99%) |
| NOV30e | 32 . . . 256 | 224/225 (99%) |
| | 1 . . . 225 | 224/225 (99%) |
| NOV30f | 30 . . . 256 | 226/227 (99%) |
| | 2 . . . 228 | 226/227 (99%) |
| NOV30g | 30 . . . 256 | 226/227 (99%) |
| | 2 . . . 228 | 226/227 (99%) |
| NOV30h | 30 . . . 256 | 226/227 (99%) |
| | 2 . . . 228 | 226/227 (99%) |
| NOV30i | 31 . . . 256 | 225/226 (99%) |
| | 10 . . . 235 | 225/226 (99%) |
| NOV30j | 1 . . . 256 | 256/256 (100%) |
| | 1 . . . 256 | 256/256 (100%) |
| NOV30k | 1 . . . 256 | 256/256 (100%) |
| | 1 . . . 256 | 256/256 (100%) |
| N0V30l | 30 . . . 256 | 226/227 (99%) |
| | 1 . . . 227 | 226/227 (99%) |
| NOV30m | 31 . . . 256 | 225/226 (99%) |
| | 10 . . . 235 | 225/226 (99%) |
| NOV30n | 30 . . . 256 | 226/227 (99%) |
| | 2 . . . 228 | 226/227 (99%) |
| NOV30o | 1 . . . 256 | 256/256 (100%) |
| | 1 . . . 256 | 256/256 (100%) |

Further analysis of the NOV30a protein yielded the following properties shown in Table 30C.

TABLE 30C
Protein Sequence Properties NOV30a

| | |
|---|---|
| SignalP analysis: | No Known Signal Sequence Predicted |
| PSORT II analysis: | PSG: a new signal peptide prediction method |
| | N-region: length 11; pos.chg 2; neg.chg 1 |
| | H-region: length 4; peak value −0.89 |
| | PSG score: −5.29 |
| | GvH: von Heijne's method for signal seq. recognition |
| | GvH score (threshold: −2.1): −6.09 |
| | possible cleavage site: between 39 and 40 |
| | >>> Seems to have no N-terminal signal peptide |
| | ALOM: Klein et al's method for TM region allocation |
| | Init position for calculation: 1 |
| | Tentative number of TMS(s) for the threshold 0.5: 0 |
| | number of TMS(s) . . . fixed |
| | PERIPHERAL Likelihood = 3.50 (at 28) |
| | ALOM score: 3.50 (number of TMSs: 0) |
| | MITDISC: discrimination of mitochondrial targeting seq |

| | | | |
|---|---|---|---|
| R content: | 6 | Hyd Moment(75): | 5.94 |
| Hyd Moment(95): | 6.68 | G content: | 10 |
| D/E content: | 2 | S/T content: | 8 |
| Score: −3.87 | | | |

Gavel: prediction of cleavage sites for mitochondrial preseq
R-2 motif at 65 SRI|TT
NUCDISC: discrimination of nuclear localization signals
pat4: none
pat7: none
bipartite: none
content of basic residues: 13.3%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
43.5%: mitochondrial
34.8%: cytoplasmic
17.4%: nuclear
4.3%: vesicles of secretory system
>> prediction for CG93735-05 is mit (k = 23)

A search of the NOV30a protein against the Geneseq database, a proprietary database that contains sequences published in patents and patent publication, yielded several homologous proteins shown in Table 30D.

TABLE 30D
Geneseq Results for NOV30a

| | | NOV30a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAG73865 | Human colon cancer antigen | 1 . . . 256 | 256/256 (100%) | e−147 |
| | protein SEQ ID NO: 4629 - | 1 . . . 256 | 256/256 (100%) | |
| | Homo sapiens, 256 aa. | | | |
| | [WO200122920-A2, | | | |
| | 05-APR-2001] | | | |
| AAM40685 | Human polypeptide SEQ ID | 22 . . . 256 | 235/235 (100%) | e−134 |
| | NO 5616 - Homo sapiens, | 5 . . . 239 | 235/235 (100%) | |
| | 239 aa. [WO200153312-A1, | | | |
| | 26-JUL-2001] | | | |
| ABB12326 | Human secreted protein | 22 . . . 256 | 235/235 (100%) | e−134 |
| | homologue, SEQ ID | 5 . . . 239 | 235/235 (100%) | |
| | NO: 2696 - Homo sapiens, | | | |
| | 239 aa. [WO200157188-A2, | | | |
| | 09-AUG-2001] | | | |
| AAB85885 | Human adenylate kinase 3 | 30 . . . 256 | 227/227 (100%) | e−129 |
| | (AK3)-like protein - Homo | 1 . . . 227 | 227/227 (100%) | |
| | sapiens, 227 aa. | | | |
| | [WO200109346-A1, | | | |
| | 08-FEB-2001] | | | |
| AAB93066 | Human protein sequence | 30 . . . 256 | 227/227 (100%) | e−129 |
| | SEQ ID NO: 11883 - Homo | 1 . . . 227 | 227/227 (100%) | |
| | sapiens, 227 aa. | | | |
| | [EP1074617-A2, | | | |
| | 07-FEB-2001] | | | |

In a BLAST search of public sequence datbases, the NOV30a protein was found to have homology to the proteins shown in the BLASTP data in Table 30E.

TABLE 30E
Public BLASTP Results for NOV30a

| | | NOV30a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9UIJ7 | GTP: AMP phosphotransferase | 31 . . . 256 | 226/226 (100%) | e−128 |
| | mitochondrial (EC 2.7.4.10) | 1 . . . 226 | 226/226 (100%) | |
| | (AK3) (Adenylate kinase 3 alpha | | | |
| | like) - Homo sapiens (Human), | | | |
| | 226 aa. | | | |
| A34442 | nucleoside-triphosphate--adenylate | 30 . . . 256 | 210/227 (92%) | e−121 |
| | kinase (EC 2.7.4.10) 3, | 1 . . . 227 | 220/227 (96%) | |
| | mitochondrial - bovine, 227 aa. | | | |
| P08760 | GTP: AMP phosphotransferase | 31 . . . 256 | 209/226 (92%) | e−120 |
| | mitochondrial (EC 2.7.4.10) | 1 . . . 226 | 219/226 (96%) | |
| | (AK3) - Bos taurus (Bovine), 226 | | | |
| | aa. | | | |
| Q9WTP7 | GTP: AMP phosphotransferase | 31 . . . 256 | 209/226 (92%) | e−118 |
| | mitochondrial (EC 2.7.4.10) | 1 . . . 226 | 217/226 (95%) | |
| | (AK3) (Adenylate kinase 3 alpha | | | |
| | like) - Mus musculus (Mouse), | | | |
| | 226 aa. | | | |
| Q95J94 | Adenylate kinase 3 - Oryctolagus | 30 . . . 256 | 209/227 (92%) | e−116 |
| | cuniculus (Rabbit), 227 aa. | 1 . . . 227 | 215/227 (94%) | |

PFam analysis predicts that the NOV30a protein contains the domains shown in the Table 30F.

TABLE 30F
Domain Analysis of NOV30a

| | | Identities/ | |
|---|---|---|---|
| | | Similarities | |
| | NOV30a Match | for the Matched | Expect |
| Pfam Domain | Region | Region | Value |
| adenylatekinase | 41 . . . 221 | 98/189 (52%) | 2.1e−110 |
| | | 170/189 (90%) | |

### Example 31

The NOV31 clone was analyzed, and the nucleotide and encoded polypeptide sequences are shown in Table 31A.

TABLE 31A
NOV31 Sequence Analysis

| | |
|---|---|
| SEQ ID No: 317 | 999 bp |

| | |
|---|---|
| NOV31a, | AGGTGAACATAACATAAAAAAATGTTCCCGGCAAATTGGACATCTGTAAAAGTATTTTTCTTCCTGG |
| CG93817-01 | |
| DNA Sequence | GATTTTTTCACTACCCCAAAGTTCAGGTCATCATATTTGCGGTGTGCTTGCTGATGTACCTGATCAC |
| | CTTGCTGGGCAACATTTTTCTGATCTCCATCACCATTCTAGATTCCCACCTGCACACCCCTATGTAC |
| | CTCTTCCTCAGCAATCTCTCCTTTCTGGACATCTGGTACTCCTCTTCTGCCCTCTCTCCAATGCTGG |
| | CAAACTTTGTTTCAGGGAGAAACACTATTTCATTCTCAGGGTGCGCCACTCAGATGTACCTCTCCCT |
| | TGCCATGGGCTCCACTGAGTGTGTGCTCCTGCCCATGATGGCATATGACCGGTATGTGGCCATCTGC |
| | AACCCCCTGAGATACCCTGTCATCATGAATAGGAGAACCTGTGTGCAGATTGCAGCTGGCTCCTGGA |
| | TGACAGGCTGTCTCACTGCCATGGTGGAAATGATGTCTGTGCTGCCACTGTCTCTCTGTGGTAATAG |
| | CATCATCAATCATTTCACTTGTGAAATTCTGGCCATCTTGAAATTGGTTTGTGTGGACACCTCCCTG |
| | GTGCAGTTAATCATGCTGGTGATCAGTGTACTTCTTCTCCCCATGCCAATGCTACTCATTTGTATCT |
| | CTTATGCATTTATCCTCGCCAGTATCCTGAGAATCAGCTCAGTGGAAGGTCGAAGTAAAGCCTTTTC |
| | AACGTGCACAGCCCACCTGATGGTGGTAGTTTTGTTCTATGGGACGGCTCTCTCCATGCACCTGAAG |
| | CCCTCCGCTGTAGATTCACAGGAAATAGACAAATTTATGGCTTTGGTGTATGCCGGACAAACCCCCA |
| | TGTTGAATCCTATCATCTATAGTCTACGGAACAAAGAGGTGAAAGTGGCCTTGAAAAAATTGCTGAT |
| | TAGAAATCATTTTAATACTGCCTTCATTTCCATCCTCAAATAACAATCACACTCATATAGA |

| | | |
|---|---|---|
| ORF Start: ATG at 22 | | ORF Stop: TAA at 979 |
| SEQ ID NO:318 | 319aa | MW at 35645.5 kD |

| | |
|---|---|
| NOV31a, | MFPANWTSVKVFFFLGFFHYPKVQVIIFAVCLLMYLITLLGNIFLISITILDSHLHTPMYLFLSNLS |
| CG93817-01 | |
| Protein Sequence | FLDIWYSSSALSPMLANFVSGRNTISFSGCATQMYLSLAMGSTECVLLPMMAYDRYVAICNPLRYPV |
| | IMNRRTCVQIAAGSWMTGCLTAMVEMMSVLPLSLCGNSIINHFTCEILAILKLVCVDTSLVQLIMLV |
| | ISVLLLPMPMLLICISYAFILASILRISSVEGRSKAFSTCTAHLMVVVLFYGTALSMHLKPSAVDSQ |
| | EIDKFMALVYAGQTPMLNPIIYSLRNKEVKVALKKLLIRNHENTAFISILK |

Further analysis of the NOV31 a protein yielded the following properties shown in Table 31B.

TABLE 31B
Protein Sequence Properties NOV31a

| | |
|---|---|
| SignalP analysis: | Cleavage site between residues 42 and 43 |
| PSORT II analysis: | PSG: a new signal peptide prediction method |
| | N-region: length 10; pos.chg 1; neg.chg 0 |
| | H-region: length 11; peak value 13.04 |
| | PSG score: 8.64 |
| | GvH: von Heijne's method for signal seq. recognition |
| | GvH score (threshold: −2.1): −0.82 |
| | possible cleavage site: between 41 and 42 |
| | >>> Seems to have a cleavable signal peptide (1 to 41) |
| | ALOM: Klein et al's method for TM region allocation |
| | Init position for calculation: 42 |
| | Tentative number of TMS(s) for the threshold 0.5: 4 |
| | INTEGRAL Likelihood = −0.96 Transmembrane 153-169 |
| | INTEGRAL Likelihood = −2.97 Transmembrane 181-197 |
| | INTEGRAL Likelihood = −10.67 Transmembrane 200-216 |
| | INTEGRAL Likelihood = −0.48 Transmembrane 240-256 |
| | PERIPHERAL Likelihood = 1.85 (at 103) |
| | ALOM score: −10.67 (number of TMSs: 4) |
| | MTOP: Prediction of membrane topology (Hartmann et al.) |
| | Center position for calculation: 20 |
| | Charge difference: −0.5 C(1.0) − N(1.5) |
| | N >= C: N-terminal side will be inside |
| | >>> membrane topology: type 3a |
| | MITDISC: discrimination of mitochondrial targeting seq |

| | | | |
|---|---|---|---|
| R content: | 0 | Hyd Moment(75): | 4.52 |
| Hyd Moment(95): | 1.36 | G content: | 2 |
| D/E content: | 1 | S/T content: | 5 |
| Score: −5.22 | | | |

Gavel: prediction of cleavage sites for mitochondrial preseq
R-2 motif at 99 GRN|TI
NUCDISC: discrimination of nuclear localization signals
pat4: none
pat7: none
bipartite: none
content of basic residues: 6.3%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
55.6%: endoplasmic reticulum
44.4%: mitochondrial
>> prediction for CG93817-01 is end (k = 9)

A search of the NOV31a protein against the Geneseq database, a proprietary database that contains sequences published in patents and patent publication, yielded several homologous proteins shown in Table 31C.

TABLE 31C
Geneseq Results for NOV31a

| | | | Identities/ | |
|---|---|---|---|---|
| | | NOV31a | Similarities | |
| | Protein/Organism/ | Residues/ | for the | |
| Geneseq | Length | Match | Matched | Expect |
| Identifier | [Patent #, Date] | Residues | Region | Value |
| AAU85329 | G-coupled olfactory | 1 . . . 319 | 319/319 | 0.0 |
| | receptor #190 - Homo | | (100%) | |
| | sapiens, 319 aa. | 1 . . . 319 | 319/319 | |
| | [WO200198526-A2, | | (100%) | |
| | 27-DEC-2001] | | | |
| ABB06654 | G protein-coupled | 1 . . . 319 | 319/319 | 0.0 |
| | receptor GPCR32a | | (100%) | |
| | protein SEQ ID | 1 . . . 319 | 319/319 | |
| | NO:118 - Homo | | (100%) | |
| | sapiens, 319 aa. | | | |
| | [WO200212343-A2, | | | |
| | 14-FEB-2002] | | | |
| AAU95674 | Human olfactory and | 1 . . . 319 | 319/319 | 0.0 |
| | pheromone G protein- | | (100%) | |
| | coupled receptor | 1 . . . 319 | 319/319 | |
| | #161 - Homo sapiens, | | (100%) | |
| | 319 aa. | | | |
| | [WO200224726-A2, | | | |
| | 28-MAR-2002] | | | |
| AAG71465 | Human olfactory | 1 . . . 319 | 319/319 | 0.0 |
| | receptor polypeptide, | | (100%) | |
| | SEQ ID NO:1146 - | 1 . . . 319 | 319/319 | |
| | Homo sapiens, 319 aa. | | (100%) | |
| | [WO200127158-A2, | | | |
| | 19-APR-2001] | | | |
| AAU24709 | Human olfactory | 1 . . . 319 | 319/319 | 0.0 |
| | receptor AOLFR208 - | | (100%) | |
| | Homo sapiens, 319 aa. | 1 . . . 319 | 319/319 | |
| | [WO200168805-A2, | | (100%) | |
| | 20-SEP-2001] | | | |

In a BLAST search of public sequence datbases, the NOV31a protein was found to have homology to the proteins shown in the BLASTP data in Table 31D.

TABLE 31D
Public BLASTP Results for NOV31a

| | | | Identities/ | |
|---|---|---|---|---|
| | | NOV31a | Similarities | |
| Protein | | Residues/ | for the | |
| Accession | Protein/Organism/ | Match | Matched | Expect |
| Number | Length | Residues | Portion | Value |
| Q8NGS4 | Seven | 1 . . . 319 | 319/319 | 0.0 |
| | transmembrane | | (100%) | |
| | helix receptor - | 1 . . . 319 | 319/319 | |
| | Homo sapiens | | (100%) | |
| | (Human), 319 aa. | | | |
| Q8VGB7 | Olfactory receptor | 1 . . . 319 | 255/319 | e−148 |
| | MOR262-2 - Mus | | (79%) | |
| | musculus (Mouse), | 1 . . . 319 | 287/319 | |
| | 319 aa. | | (89%) | |
| Q8VGI0 | Olfactory receptor | 5 . . . 305 | 200/301 | e−114 |
| | MOR262-1 - Mus | | (66%) | |
| | musculus (Mouse), | 5 . . . 304 | 248/301 | |
| | 313 aa. | | (81%) | |
| CAD37524 | Sequence 71 from | 5 . . . 305 | 196/301 | e−110 |
| | Patent | | (65%) | |
| | WO0224726 - | 34 . . . 333 | 243/301 | |
| | Homo sapiens | | (80%) | |
| | (Human), 345 aa. | | | |
| Q8NGT1 | Seven | 5 . . . 305 | 196/301 | e−110 |
| | transmembrane | | (65%) | |
| | helix receptor - | 5 . . . 304 | 243/301 | |
| | Homo sapiens | | (65%) | |
| | (Human), 316 aa. | | | |

PFam analysis predicts that the NOV31a protein contains the domains shown in the Table 31E.

TABLE 31E
Domain Analysis of NOV31a

| | NOV31a Match | Identities/Similarities | Expect |
|---|---|---|---|
| Pfam Domain | Region | for the Matched Region | Value |
| 7tm_1 | 41 . . . 290 | 54/268 (20%) | 6e−37 |
| | | 171/268 (64%) | |

### Example 32

The NOV32 clone was analyzed, and the nucleotide and encoded polypeptide sequences are shown in Table 32A.

TABLE 32A
NOV32 Sequence Analysis

| | |
|---|---|
| SEQ ID NO: 319 | 1041 bp |

| | |
|---|---|
| NOV32a, | AAATTCCGGCCAAGATGGCAGCAATGAGGAAGGCGCTTCCGCGGCGACTGGTGGGCTTGGCGTCCCT |
| CG96859-03 | |
| DNA Sequence | CCGGGCTGTCAGCACCTCATCTATGGGCACTTTACCAAAGCGGGTGAAAATTGTGGAAGTTGGTCCC |
| | CGAGATGGACTACAAAATGAAAAGAATATCGTATCTACTCCAGTGAAAATCAAGCTGATAGACATGC |
| | TTTCTGAAGCAGGACTCTCTGTTATAGAAACCACCAGCTTTGTGTCTCCTAAGTGGGTTCCCCAGAT |
| | GGGTGACCACACTGAAGTCTTGAAGGGCATTCAGAAGTTTCCTGGCATCAACTACCCAGTCCTGACC |
| | CCAAATTTGAAAGGCTTCGAGGCAGCGGTTGCTGCTGGAGCCAAGGAAGTAGTCATCTTTGGAGCTG |
| | CCTCAGAGCTCTTCACCAAGAAGAACATCAATTGTTCCATAGAGGAGAGTTTTCAGAGGTTTGACGC |
| | AATCCTGAAGGCAGCGCAGTCAGCCAATATTTCTGTGCGGGGGTACGTCTCCTGTGCTCTTGGCTGC |
| | CCTTATGAAGGGAAGATCTCCCCAGCTAAAGTAGCTGAGGTCACCAAGAAGTTCTACTCAATGGGCT |
| | GCTACGAGATCTCCCTGGGGGACACCATTGGTGTGGGCACCCCAGGGATCATGAAAGACATGCTGTC |
| | TGCTGTCATGCAGGAAGTGCCTCTGGCTGCCCTGGCTGTCCACTGCCATGACACCTATGGTCAAGCC |
| | CTGGCCAACACCTTGATGGCCCTGCAGATGGGAGTGAGTGTCGTGGACTCTTCTGTGGCAGGACTTG |
| | GAGGCTGTCCCTACGCACAGGGGGCATCAGGAAACTTGGCCACAGAAGACCTGGTCTACATGCTAGA |
| | GGGCTTGGGCATTCACACGGGTGTGAATCTCCAGAAGCTTCTGGAAGCTGGAAACTTTATCTGTCAA |
| | GCCCTGAACAGAAAAACTAGCTCCAAAGTGGCTCAGGCTACCTGTAAACTCTGAGCCCCTTGCCCAC |
| | CTGAAGGCCTGGGGATGATGTGGAAATAAGGGGCAT |

| | | |
|---|---|---|
| ORF Start: ATG at 15 | | ORF Stop: TGA at 990 |
| SEQ ID NO: 320 | 325 aa | MW at 34359.8 kD |

| | |
|---|---|
| NOV32a, | MAAMRKALPRRLVGLASLRAVSTSSMGTLPKRVKIVEVGPRDGLQNEKNIVSTPVKIKLIDMLSEAG |
| CG96859-03 | |
| Protein Sequence | LSVIETTSFVSPKWVPQMGDHTEVLKGIQKFPGINYPVLTPNLKGFEAAVAAGAKEVVIFGAASELF |
| | TKKNINCSIEESFQRFDAILKAAQSANISVRGYVSCALGCPYEGKISPAKVAEVTKKFYSMGCYEIS |
| | LGDTIGVGTPCIMKDMLSAVMQEVPLAALAVHCHDTYGQALANTLMALQMGVSVVDSSVAGLGGCPY |
| | AQGASGNLATEDLVYMLEGLGIHTGVNLQKLLEAGNFICQALNRKTSSKVAQATCKL |

| | |
|---|---|
| SEQ ID NO: 321 | 969 bp |

| | |
|---|---|
| NOV32b, | TAACTTTATTATTAAAAATTAAAGAGGTATATATTAATGTATCGATTAAATAAGGAGGAATAAACCA |
| 223316960 DNA | |
| Sequence | TGGGCACTTTACCAAAGCGGGTGAAAATTGTGGAAGTTGGTCCCCGAGATGGACTACAAAATGAAAA |
| | GAATATCGTATCTACTCCAGTGAAAATCAAGCTGATAGACATGCTTTCTGAAGCAGGACTCTCTGTT |
| | ATAGAAACCACCAGCTTTGTGTCTCCTAAGTGGGTTCCCCAGATGGGTGACCACACTGAAGTCTTGA |
| | AGGGCATTCAGAAGTTTCCTGGCATCAACTACCCAGTCCTGACCCCAAATTTGAAAGGCTTCGAGGC |
| | AGCGGTTGCTGCTGGAGCCAAGGAAGTAGTCATCTTTGGAGCTGCCTCAGAGCTCTTCACCAAGAAG |
| | AACATCAATTGTTCCATAGAGGAGACTTTTCAGAGGTTTGACGCAATCCTGAAGGCAGCGCAGTCAG |
| | CCAATATTTCTGTGCGGGGGTACGTCTCCTGTGCTCTTGGCTGCCCTTATGAAGGGAAGATCTCCCC |
| | AGCTAAAGTAGCTGAGGTCACCAAGAAGTTCTACTCAATGGGCTGCTACGAGATCTCCCTGGGGGAC |
| | ACCATTGGTGTGGGCACCCCAGGGATCATGAAAGACATGCTGTCTGCTGTCATGCAGGAAGTGCCTC |
| | TGGCTGCCCTGGCTGTCCACTGCCATGACACCTATGGTCAAGCCCTGGCCAACACCTTGATGGCCCT |
| | GCAGATGGGAGTGAGTGTCGTGGACTCTTCTGTGGCAGGACTTGGAGGCTGTCCCTACGCACAGGGG |
| | GCATCAGGAAACTTGGCCACAGAAGACCTGGTCTACATGCTAGAGGGCTTGGGCATTCACACGGGTG |
| | TGAATCTCCAGAAGCTTCTGGAAGCTGGAAACTTTATCTGTCAAGCCCTGAACAGAAAAACTAGCTC |
| | CAAAGTGGCTCAGGCTACCTGTAAACTCTGA |

| | | |
|---|---|---|
| ORF Start: at 64 | | ORF Stop: TGA at 967 |
| SEQ ID NO: 322 | 301 aa | MW at 31835.7 kD |

| | |
|---|---|
| NOV32b, | TMGTLPKRVKIVEVGPRDGLQNEKNIVSTPVKIKLIDMLSEAGLSVIETTSFVSPKWVPQMGDHTEV |
| 223316960 | |
| Protein Sequence | LKGIQKFPGINYPVLTPNLKGFEAAVAAGAKEVVIFGAASELFTKKNINCSIEESFQRFDAILKAAQ |
| | SANISVRGYVSCALGCPYEGKISPAKVAEVTKKFYSMGCYEISLGDTIGVGTPGIMKDMLSAVMQEV |
| | PLAALAVHCHDTYGQALANTLMALQMGVSVVDSSVAGLGGCPYAQGASGNLATEDLVYMLEGLGIHT |
| | GVNLQKLLEAGNFICQALNRKTSSKVAQATCKL |

| | |
|---|---|
| SEQ ID NO: 323 | 987 bp |

| | |
|---|---|
| NOV32c, | TAACTTTATTATTAAAAATTAAAGAGGTATATATTAATGTATCGATTAAATAAGGAGGAATAAACCA |
| 223316987 DNA | |
| Sequence | TGGGCCACCATCACCACCATCACACTTTACCAAAGCGGGTGAAAATTGTGGAAGTTGGTCCCCGAGA |
| | TGGACTACAAAATGAAAAGAATATCGTATCTACTCCAGTGAAAATCAAGCTGATAGACATGCTTTCT |
| | GAAGCAGGACTCTCTGTTATAGAAACCACCAGCTTTGTGTCTCCTAAGTGGGTTCCCCAGATGGGTG |
| | ACCACACTGAAGTCTTGAAGGGCATTCAGAAGTTTCCTGGCATCAACTACCCAGTCCTGACCCCAAA |
| | TTTGAAAGGCTTCGAGGCAGCGGTTGCTGCTGGAGCCAAGGAAGTAGTCATCTTTGGAGCTGCCTCA |
| | GAGCTCTTCACCAAGAAGAACATCAATTGTTCCATAGAGGAGAGTTTTCAGAGGTTTGACGCAATCC |
| | TGAAGGCAGCGCAGTCAGCCAATATTTCTGTGCGGGGGTACGTCTCCTGTGCTCTTGGCTGCCCTTA |
| | TGAAGGGAAGATCTCCCCAGCTAAAGTAGCTGAGGTCACCAAGAAGTTCTACTCAATGGGCTGCTAC |
| | GAGATCTCCCTGGGGGACACCATTGGTGTGGGCACCCCAGGGATCATGAAAGACATGCTGTCTGCTG |
| | TCATGCAGGAAGTGCCTCTGGCTGCCCTGGCTGTCCACTGCCATGACACCTATGGTCAAGCCCTGGC |
| | CAACACCTTGATGGCCCTGCAGATGGGAGTGAGTGTCGTGGACTCTTCTGTGGCAGGACTTGGAGGC |
| | TGTCCCTACGCACAGGGGGCATCAGGAAACTTGGCCACAGAAGACCTGGTCTACATGCTAGAGGGCT |
| | TGGGCATTCACACGGGTGTGAATCTCCAGAAGCTTCTGGAAGCTGGAAACTTTATCTGTCAAGCCCT |
| | GAACAGAAAAACTAGCTCCAAAGTGGCTCAGGCTACCTGTAAACTCTGA |

| | | |
|---|---|---|
| ORF Start: at 64 | | ORF Stop: TGA at 985 |
| SEQ ID NO: 324 | 307 aa | MW at 32658.6 kD |

| | |
|---|---|
| NOV32c, | TMGHHHHHHTLPKRVKIVEVGPRDGLQNEKNIVSTPVKIKLIDMLSEAGLSVIETTSFVSPKWVPQM |
| 223316987 | |
| Protein Sequence | GDHTEVLKGIQKFPGINYPVLTPNLKGFEAAVAAGAKEVVIFGAASELFTKKNINCSIEESFQRFDA |
| | ILKAAQSANISVRGYVSCALGCPYEGKISPAKVAEVTKKFYSMGCYEISLGDTIGVGTPGIMKDMLS |
| | AVMQEVPLAALAVHCHDTYGQALANTLMALQMGVSVVDSSVAGLGGCPYAQGASGNLATEDLVYMLE |
| | GLGIHTGVNLQKLLEAGNFICQALNRKTSSKVAQATCKL |

| | |
|---|---|
| SEQ ID NO: 325 | 1568 bp |

| | |
|---|---|
| NOV32d, | GAATTCCGGCCAAGATGGCAGCAATGAGGAAGGCGCTTCCGCGGCGACTGGTGGGCTTGGCGTCCCT |
| CG96859-01 | |
| DNA Sequence | CCGGGCTGTCAGCACCTCATCTATGGGCACTTTACCAAAGCGGGTGAAAATTGTGGAAGTTGGTCCC |
| | CGAGATGGACTACAAAATGAAAAGAATATCGTATCTACTCCAGTGAAAATCAAGCTGATAGACATGC |
| | TTTCTGAAGCAGGACTCTCTGTTATAGAAACCACCAGCTTTGTGTCTCCTAAGTGGGTTCCCCAGAT |
| | GGGTGACCACACTGAAGTCTTGAAGGGCATTCAGAAGTTTCCTGGCATCAACTACCCAGTCCTGACC |
| | CCAAATTTGAAAGGCTTCGAGGCAGCGGTTGCTGCTGGAGCCAAGGAAGTAGTCATCTTTGGAGCTG |
| | CCTCAGAGCTCTTCACCAAGAAGAACATCAATTGTTCCATAGAGGAGAGTTTTCAGAGGTTTGACGC |
| | AATCCTGAAGGCAGCGCAGTCAGCCAATATTTCTGTGCGGGGGTACGTCTCCTGTGCTCTTGGCTGC |
| | CCTTATGAAGGGAAGATCTCCCCAGCTAAAGTAGCTGAGGTCACCAAGAAGTTCTACTCAATGGGCT |
| | GCTACGAGATCTCCCTGGGGGACACCATTGGTGTGGGCACCCCAGGGATCATGAAAGACATGCTATC |
| | TGCTGTCATGCAGGAAGTGCCTCTGGCTGCCCTGGCTGTCCACTGCCATGACACCTATGGTCAAGCC |
| | CTGACCAACACCTTGATGGCCCTGCAGATGGGAGTGAGTGTCGTGGACTCTTCTGTGGCAGGACTTG |
| | GAGGCTGTCCCTACGCACAGGGGGCATCAGGAAACTTGGCCACAGAAGACCTGGTCTACATGCTAGA |
| | GGGCTTGGGCATTCACACGGGTGTGAATCTCCAGAAGCTTCTGGAAGCTGGAAACTTTATCTGTCAA |
| | GCCCTGAACAGAAAAACTAGCTCCAAAGTGGCTCAGGCTACCTGTAAACTCTGAGCCCCTTGCCCAC |
| | CTGAAGCCCTGGGGATGATGTGGAAATAGGGGCACACACAGATGATTCATGGATGGGGACATGGAAA |
| | TGAGAATAGGTTAAATGGTGCAGGTACCTCATAGCCAGCTCTACACAGAGGTCTCTCCTGGCAGAAA |
| | GCAGGCGAAGGGCAGGAGGAGCTGCTTGGCAGAAGGACCTCCTGCCCAGACCTGAGGAGTGAGAGGC |
| | TTTGAGGGCTGAAGTCTCCCTTTGTTACGGACCCTGGCCCAGGAGTTGAATGCCTGAGGACGTGTGG |
| | GAACCCCGTTCCCTACTTAGCATGATCCTTGAGTCTCCTCTCTGGATGGAATCCGCGAGCTGGCCAC |
| | CTGGCCACCCTCTACACGGCTCCACCCTGCCATGGCCGTGGGGCCCTTGCTCTCTGACTTCTCAGGA |
| | CACAGGTCATGGAGGTTCTTCCCAAGCTGGCAGAGGCCATTTGTGGAAAGTGGAGAGCTACGTGGTG |
| | GCCGTCTGCCAACTCCAGCATCTCTGGAAAATCTCCACGCTGAATGTGATTTTTGAAAACAGCTTAT |
| | GTAATTAAAGGTTGAATGGCACATCAT |

| | | |
|---|---|---|
| ORF Start: ATG at 15 | | ORF Stop: TGA at 990 |
| SEQ ID NO: 326 | 325 aa | MW at 34389.8 kD |

| | |
|---|---|
| NOV32d, | MAAMRKALPRRLVGLASLRAVSTSSMGTLPKRVKIVEVGPRDGLQNEKNIVSTPVKIKLIDMLSEAG |
| CG96859-01 | |
| Protein Sequence | LSVIETTSFVSPKWVPQMGDHTEVLKGIQKFPGINYPVLTPNLKGFEAAVAAGAKEVVIFGAASELF |
| | TKKNINCSIEESFQRFDAILKAAQSANISVRGYVSCALGCPYEGKISPAKVAEVTKKFYSMGCYEIS |
| | LGDTIGVGTPGIMKDMLSAVMQEVPLAALAVHCHDTYGQALTNTLMALQMGVSVVDSSVAGLGGCPY |
| | AQGASGNLATEDLVYMLEGLGIHTGVNLQKLLEAGNFICQALNRKTSSKVAQATCKL |

| | |
|---|---|
| SEQ ID NO: 327 | 1355 bp |

| | |
|---|---|
| NOV32e, | GAATTCCGGCCAAGATGGCAGCAATGAGGAAGGCGCTTCCGCGGCGACTGGTGGGCTTGGCGTCCCT |
| CG96859-02 | |
| DNA Sequence | CCGGGCTGTCAGCACCTCATCTATGGGCACTTTACCAAAGCGGGTGAAAATTGTGGAAGTTGGTCCC |
| | CGAGATGGACTACAAAATGAAAAGAATATCGTATCTACTCCAGTGAAAATCAAGCTGATAGACATGC |
| | TTTCTGAAGCAGGACTCTCTGTTATAGAAACCACCAGCTTTGTGTCTCCTAAGTGGGTTCCCCAGAT |
| | GGGTGACCACACTGAAGTCTTGAAGGGCATTCAGAAGTTTCCTGGCATCAACTACCCAGTCCTGACC |
| | CCAAATTTGAAAGGCTTCGAGGCAGCGGTCACCAAGAAGTTCTACTCAATGGGCTGCTACGAGATCT |
| | CCCTGGGGGACACCATTGGTGTGGGCACCCCAGGGATCATGAAAGACATGCTGTCTGCTGTCATGCA |
| | GGAAGTGCCTCTGGCTGCCCTGGCTGTCCACTGCCATGACACCTATGGTCAAGCCCTGGCCAACACC |
| | TTGATGGCCCTGCAGATGGGAGTGAGTGTCGTGGACTCTTCTGTGGCAGGACTTGGAGGCTGTCCCT |
| | ACGCACAGGGGGCATCAGGAAACTTGGCCACAGAAGACCTGGTCTACATGCTAGAGGGCTTGGGCAT |
| | TCACACGGGTGTGAATCTCCAGAAGCTTCTGGAAGCTGGAAACTTTATCTGTCAAGCCCTGAACAGA |
| | AAAACTAGCTCCAAAGTGGCTCAGGCTACCTGTAAACTCTGAGCCCCTTGCCCACCTGAAGCCCTGG |
| | GGATGATGTGGAAATAGGGGCACACACAGATGATTCATGGATGGGGACATGGAAATGAGAATAGGTT |
| | AAATGGTGCAGGTACCTCATAGCCAGCTCTACACAGAGGTCTCTCCTGGCAGAAAGCAGGCGAAGGG |
| | CAGGAGGAGCTGCTTGGCAGAAGGACCTCCTGCCCAGACCTGAGGAGTGAGAGGCTTTGAGGGCTGA |
| | AGTCTCCCTTTGTTACGGACCCTGGCCCAGGAGTTGAATGCCTGAGGACGTGTGGGAACCCCGTTCC |
| | CTACTTAGCATGATCCTTGAGTCTCCTCTCTGGATGGAATCCGCGAGCTGGCCACCTGGCCACCCTC |
| | TACACGGCTCCACCCTGCCATGGCCGTGGGGCCCTTGCTCTCTGACTTCTCAGGACACAGGTCATGG |
| | AGGTTCTTCCCAAGCTGGCAGAGGCCATTTGTGGAAAGTGGAGAGCTACGTGGTGGCCGTCTGCCAA |
| | CTCCAGCATCTCTGGAAAATCTCCACGCTGAATGTGATTTTTGAAAACAGCTTATGTAATTAAAGGT |
| | TGAATGGCACATCAT |

| | | |
|---|---|---|
| ORF Start: ATG at 15 | | ORF Stop: TGA at 777 |
| SEQ ID NO: 328 | 254 aa | MW at 26909.3 kD |

| | |
|---|---|
| NOV32e, | MAAMRKALPRRLVGLASLRAVSTSSMGTLPKRVKIVEVGPRDGLQNEKNIVSTPVKIKLIDMLSEAG |
| CG96859-02 | |
| Protein Sequence | LSVIETTSFVSPKWVPQMGDHTEVLKGIQKFPGINYPVLTPNLKGFEAAVTKKFYSMGCYEISLGDT |
| | IGVGTPGIMKDMLSAVMQEVPLAALAVHCHDTYGQALANTLMALQMGVSVVDSSVAGLGGCPYAQGA |
| | SGNLATEDLVYMLEGLGIHTGVNLQKLLEAGNFICQALNRKTSSKVAQATCKL |

| | |
|---|---|
| SEQ ID NO: 329 | 788 bp |

| | |
|---|---|
| NOV32f, | GATGGCAGCAATGAGGAAGGCGCTTCCGCGGCGACTGGTGGGCTTGGCGTCCCTCCGGGCTGTCAGC |
| CG96859-04 | |
| DNA Sequence | ACCTCATCTATGGGCACTTTACCAAAGCGGGTGAAAATTGTGGAAGTTGGTCCCCGAGATGGACTAC |
| | AAAATGAAAGGAATATCGTATCTACTCCAGTGAAAATCAAGCTGATAGACATGCTTTCTGAAGCAGG |
| | ACTCTCTGTTATAGAAACCACCAGCTTTGTGTCTCCTAAGTGGGTTCCCCAGATGGGTGACCACACT |
| | GAAGTCTTGAAGGGCATTCAGAAGTTTCCTGGCATCAACTACCCAGTCCTGACCCCAAATTTGAAAG |
| | GCTTCGAGGCAGCGGTCACCAAGAAGTTCTACTCAATGGGCTGCTACGAGATCTCCCTGGGGGACAC |
| | CATTGGTGTGGGCACCCCAGGGATCATGAAAGACATGCTGTCTGCTGTCATGCAGGAAGTGCCTCTG |
| | GCTGCCCTGGCTGTCCACTGCCATGACACCTATGGTCAAGCCCTGGCCAACACCTTGATGGCCCTGC |
| | AGATGGGAGTGAGTGTCGTGGACTCTTCTGTGGCAGGACTTGGAGGCTGTCCCTACGCACAGGGGGC |
| | ATCAGGAAACTTGGCCACAGAAGACCTGGTCTACATGCTAGAGGGCTTGGGCATTCACACGGGTGTG |
| | AATCTCCAGAAGCTTCTGGAAGCTGGAAACTTTATCTGTCAAGCCCTGAACAGAAAAACTAGCTCCA |
| | AAGTGGCTCAGGCTACCTGTAAACTCTGAGCCCCTTGCCCACCTGAAGCCC |

| | | |
|---|---|---|
| ORF Start: ATG at 2 | | ORF Stop: TGA at 764 |
| SEQ ID NO: 330 | 254 aa | MW at 26937.3 kD |

| | |
|---|---|
| NOV32f, | MAAMRKALPRRLVGLASLRAVSTSSMGTLPKRVKIVEVGPRDGLQNERNIVSTPVKIKLIDMLSEAG |
| CG96859-04 | |
| Protein | LSVIETTSFVSPKWVPQMGDHTEVLKGLQKFPGINYPVLTPNLKGFEAAVTKKFYSMGCYEISLGDT |
| Sequence | |
| | IGVGTPGIMKDMLSAVMQEVPLAALAVHCHDTYGQALANTLMALQMGVSVVDSSVAGLGGCPYAQGA |
| | SGNLATEDLVYMLEGLGIHTGVNLQKLLEAGNFICQALNRKTSSKVAQATCKL |

| | |
|---|---|
| SEQ ID NO: 331 | 893 bp |

| | |
|---|---|
| NOV32g, | GATGGCAGCAATGAGGAAGGCGCTTCCGCGGCGACTGGTGGGCTTGGCGTCCCTCCGGGCTGTCAGC |
| CG96859-05 | |
| DNA Sequence | ACCTTATCTATGGGCACTTTACCAAAGCGGGTGAAAATTGTGGAAGTTGGTCCCCGAGATGGACTAC |
| | AAAATGAAAAGAATATCGTATCTACTCCAGTGAAAATCAAGCTGATAGACATGCTTTCTGAAGCAGG |
| | ACTCTCTGTTATAGAAACCACCAGCTTTGTGTCTCCTAAGTGGGTTCCCCAGATGGGTGACCACACT |
| | GAAGTCTTGAAGGGCATTCAGAAGTTTCCTGGCATCAACTACCCAGTCCTGACCCCAAATTTGAAAG |
| | GCTTCGAGGCAGCGGTTGCTGCTGGAGCCAAGGAAGTAGTCATCTTTGGAGCTGCCTCAGAGCTCTT |
| | CACCAAGAAGAACATCAATTGTTCCATAGAGGAGAGTTTTCAGAGGTTTGACGCAATCCTGAAGGCA |
| | GCGCAGTCAGCCAATATTTCTGTGCGGGGGTACGTCTCCTGTGCTCTTGGCTGCCCTTATGAAGGGA |
| | AGATCTCCCCAGCTAAAGTAGCTGAGGAAGTGCCTCTGGCTGCCCTGGCTGTCCACTGCCATGACAC |
| | CTATGGTCAAGCCCTGGCCAACACCTTGATGGCCCTGCAGATGGGAGTGAGTGTCGTGGACTCTTCT |
| | GTGGCAGGACTTGGAGGCTGTCCCTACGCACAGGGGGCATCAGGAAACTTGGCCACAGAAGACCTGG |
| | TCTACATGCTAGAGGGCTTGGGCATTCACACGGGTGTGAATCTCCAGAAGCTTCTGGAAGCTGGAAA |
| | CTTTATCTGTCAAGCCCTGAACAGAAAAACTAGCTCCAAAGTGGCTCAGGCTACCTGTAAACTCTGA |
| | GCCCCTTGCCCACCTGAAGCCC |

| | | |
|---|---|---|
| ORF Start: ATG at 2 | | ORF Stop: TGA at 869 |
| SEQ ID NO: 332 | 289 aa | MW at 30531.3 kD |

| | |
|---|---|
| NOV32g, | MAAMRKALPRRLVGLASLRAVSTLSMGTLPKRVKIVEVGPRDGLQNEKNIVSTPVKIKLIDMLSEAG |
| CG96859-05 | |
| Protein Sequence | LSVIETTSFVSPKWVPQMGDHTEVLKGIQKFPGINYPVLTPNLKGFEAAVAAGAKEVVIFGAASELF |
| | TKKNINCSIEESFQRFDAILKAAQSANISVRGYVSCALGCPYEGKISPAKVAEEVPLAALAVHCHDT |
| | YGQALANTLMALQMGVSVVDSSVAGLGGCPYAQGASGNLATEDLVYMLEGLGIHTGVNLQKLLEAGN |
| | FICQALNRKTSSKVAQATCKL |

| | |
|---|---|
| SEQ ID NO: 333 | 1353 bp |

| | |
|---|---|
| NOV32h, | CCCCAAAATTCGTAACAACTCCGCCCCATTGACGCAAATGGGCGGTAGGCGTGTACGGTGGGAGGTC |
| CG96859-06 | |
| DNA Sequence | TATATAAGCAGAGCTCTCTGGCTAACTAGAGAACCCACTGCTTACTGGCTTATCGAAATTAATACGA |
| | CTCACTATAGGGAGACCCAAGCTGGCTAGCGTTTAAACTTAAGCTTGGTACCGAGCTCGGATCCACC |
| | ATGGCAGCAATGAGGAAGGCGCTTCCGCGGCGACTGGTGGGCTTGGCGTCCCTCCGGGCTGTCAGCA |
| | CCTCATCTATGGGCACTTTACCAAAGCGGGTGAAAATTGTGGAAGTTGGTCCCCGAGATGGACTACA |
| | AAATGAAAAGAATATCGTATCTACTCCAGTGAAAATCAAGCTGATAGACATGCTTTCTGAAGCAGGA |
| | CTCTCTGTTATAGAAACCACCAGCTTTGTGTCTCCTAAGTGGGTTCCCCAGATGGGTGACCACACTG |
| | AAGTCTTGAAGGGCATTCAGAAGTTTCCTGGCATCAACTACCCAGTCCTGACCCCAAATTTGAAAGG |
| | CTTCGAGGCAGCGGTTGCTGCTGGAGCCAAGGAAGTAGTCATCTTTGGAGCTGCCTCAGAGCTCTTC |
| | ACCAAGAAGAACATCAATTGTTCCATAGAGGAGAGTTTTCAGAGGTTTGACGCAATCCTGAAGGCAG |
| | CGCAGTCAGCCAATATTTCTGTGCGGGGGTACGTCTCCTGTGCTCTTGGCTGCCCTTATGAAGGGAA |
| | GATCTCCCCAGCTAAAGTAGCTGAGGTCACCAAGAAGTTCTACTCAATGGGCTGCTACGAGATCTCC |
| | CTGGGGGACACCATTGGTGTGGGCACCCCAGGGATCATGAAAGACATGCTGTCTGCTGTCATGCAGG |
| | AAGTGCCTCTGGCTGCCCTGGCTGTCCACTGCCATGACACCTATGGTCAAGCCCTGGCCAACACCTT |
| | GATGGCCCTGCAGATGGGAGTGAGTGTCGTGGACTCTTCTGTGGCAGGACTTGGAGGCTGTCCCCAC |
| | GCACAGGGGGCATCAGGAAACTTGGCCACAGAAGACCTGGTCTACATGCTAGAGGGCTTGGGCATTC |
| | ACACGGGTGTGAATCTCCAGAAGCTTCTGGAAGCTGGAAACTTTATCTGTCAAGCCCTGAACAGAAA |
| | AACTAGCTCCAAAGTGGCTCAGGCTACCTGTAAACTCTGAGCGGCCGCTCGAGTCTAGAGGGCCCGT |
| | TTAAACCCGCTGATCAGCCTCGACTGTGCCTTCTAGTTGCCAGCCATCTGTTGTTTGCCCCTCCCCC |
| | GTGCCTTCCTTGACCCTGGAAGGTGCCACTCCCACTGTCCTTTCCTAATAAAATGAGGAAATTGCAT |
| | CGCATTGTCTGAG |

| | | |
|---|---|---|
| ORF Start: | ATG at 202 | ORF Stop: at 1171 |
| SEQ ID NO: 334 | 323 aa | MW at 34118.4 kD |

| | |
|---|---|
| NOV32h, | MAAMRKALPRRLVGLASLRAVSTSSMGTLPKRVKIVEVGPRDGLQNEKNIVSTPVKIKLIDMLSEAG |
| CG96859-06 | |
| Protein Sequence | LSVIETTSFVSPKWVPQMGDHTEVLKGIQKFPGINYPVLTPNLKGFEAAVAAGAKEVVIFGAASELF |
| | TKKNINCSIEESFQRFDAILKAAQSANISVRGYVSCALGCPYEGKISPAKVAEVTKKFYSMGCYEIS |
| | LGDTIGVGTPGIMKDMLSAVMQEVPLAALAVHCHDTYGQALANTLMALQMGVSVVDSSVAGLGGCPY |
| | AQGASGNLATEDLVYMLEGLGIHTGVNLQKLLEAGNFICQALNRKTSSKVAQATC |

| | |
|---|---|
| SEQ ID NO: 335 | 969 bp |

| | |
|---|---|
| NOV32i, | TAACTTTATTATTAAAAATTAAAGAGGTATATATTAATGTATCGATTAAATAAGGAGGAATAAACCA |
| CG96859-07 | |
| DNA Sequence | TGGGCACTTTACCAAAGCGGGTGAAAATTGTGGAAGTTGGTCCCCGAGATGGACTACAAAATGAAAA |
| | GAATATCGTATCTACTCCAGTGAAAATCAAGCTGATAGACATGCTTTCTGAAGCAGGACTCTCTGTT |
| | ATAGAAACCACCAGCTTTGTGTCTCCTAAGTGGGTTCCCCAGATGGGTGACCACACTGAAGTCTTGA |
| | AGGGCATTCAGAAGTTTCCTGGCATCAACTACCCAGTCCTGACCCCAAATTTGAAAGGCTTCGAGGC |
| | AGCGGTTGCTGCTGGAGCCAAGGAAGTAGTCATCTTTGGAGCTGCCTCAGAGCTCTTCACCAAGAAG |
| | AACATCAATTGTTCCATAGAGGAGAGTTTTCAGAGGTTTGACGCAATCCTGAAGGCAGCGCAGTCAG |
| | CCAATATTTCTGTGCGGGGGTACGTCTCCTGTGCTCTTGGCTGCCCTTATGAAGGGAAGATCTCCCC |
| | AGCTAAAGTAGCTGAGGTCACCAAGAAGTTCTACTCAATGGGCTGCTACGAGATCTCCCTGGGGGAC |
| | ACCATTGGTGTGGGCACCCCAGGGATCATGAAAGACATGCTGTCTGCTGTCATGCAGGAAGTGCCTC |
| | TGGCTGCCCTGGCTGTCCACTGCCATGACACCTATGGTCAAGCCCTGGCCAACACCTTGATGGCCCT |
| | GCAGATGGGAGTGAGTGTCGTGGACTCTTCTGTGGCAGGACTTGGAGGCTGTCCCTACGCACAGGGG |
| | GCATCAGGAAACTTGGCCACAGAAGACCTGGTCTACATGCTAGAGGGCTTGGGCATTCACACGGGTG |
| | TGAATCTCCAGAAGCTTCTGGAAGCTGGAAACTTTATCTGTCAAGCCCTGAACAGAAAAACTAGCTC |
| CAAAGTGGCTCAGGCTACCTGTAAACTCTGA | |

| | | |
|---|---|---|
| ORF Start: at 64 | | ORF Stop: TGA at 967 |
| SEQ ID NO: 336 | 301 aa | MW at 31835.7 kD |

| | |
|---|---|
| NOV32i, | TMGTLPKRVKIVEVGPRDGLQNEKNIVSTPVKIKLIDMLSEAGLSVIETTSFVSPKWVPQMGDHTEV |
| CG96859-07 | |
| Protein | LKGIQKFPGINYPVLTPNLKGFEAAVAAGAKEVVIFGAASELFTKKNINCSIEESFQRFDAILKAAQ |
| Sequence | |
| | SANISVRGYVSCALGCPYEGKISPAKVAEVTKKFYSMGCYEISLGDTIGVGTPGIMKDMLSAVMQEV |
| | PLAALAVHCHDTYGQALANTLMALQMGVSVVDSSVAGLGGCPYAQGASGNLATEDLVYMLEGLGIHT |
| | GVNLQKLLEAGNFICQALNRKTSSKVAQATCKL |

| | |
|---|---|
| SEQ ID NO: 337 | 969 bp |

| | |
|---|---|
| NOV32j, | TAACTTTATTATTAAAAATTAAAGAGGTATATATTAATGTATCGATTAAATAAGGAGGAATAAACCA |
| CG96859-08 | |
| DNA Sequence | TGGGCACTTTACCAAAGCGGGTGAAAATTGTGGAAGTTGGTCCCCGAGATGGACTACAAAATGAAAA |
| | GAATATCGTATCTACTCCAGTGAAAATCAAGCTGATAGACATGCTTTCTGAAGCAGGACTCTCTGTT |
| | ATAGAAACCACCAGCTTTGTGTCTCCTAAGTGGGTTCCCCAGATGGGTGACCACACTGAAGTCTTGA |
| | AGGGCATTCAGAAGTTTCCTGGCATCAACTACCCAGTCCTGACCCCAAATTTGAAAGGCTTCGAGGC |
| | AGCGGTTGCTGCTGGAGCCAAGGAAGTAGTCATCTTTGGAGCTGCCTCAGAGCTCTTCACCAAGAAG |
| | AACATCAATTGTTCCATAGAGGAGAGTTTTCAGAGGTTTGACGCAATCCTGAAGGCAGCGCAGTCAG |
| | CCAATATTTCTGTGCGGGGGTACGTCTCCTGTGCTCTTGGCTGCCCTTATGAAGGGAAGATCTCCCC |
| | AGCTAAAGTAGCTGAGGTCACCAAGAAGTTCTACTCAATGGGCTGCTACGAGATCTCCCTGGGGGAC |
| | ACCATTGGTGTGGGCACCCCAGGGATCATGAAAGACATGCTGTCTGCTGTCATGCAGGAAGTGCCTC |
| | TGGCTGCCCTGGCTGTCCACTGCCATGACACCTATGGTCAAGCCCTGGCCAACACCTTGATGGCCCT |
| | GCAGATGGGAGTGAGTGTCGTGGACTCTTCTGTGGCAGGACTTGGAGGCTGTCCCTACGCACAGGGG |
| | GCATCAGGAAACTTGGCCACAGAAGACCTGGTCTACATGCTAGAGGGCTTGGGCATTCACACGGGTG |
| | TGAATCTCCAGAAGCTTCTGGAAGCTGGAAACTTTATCTGTCAAGCCCTGAACAGAAAAACTAGCTC |
| | CAAAGTGGCTCAGGCTACCTGTAAACTCTGA |

| | | | |
|---|---|---|---|
| ORF Start: at 64 | | | ORF Stop: TGA at 967 |
| SEQ ID NO: 338 | 301 aa | MW at 31835.7 kD | |

| | |
|---|---|
| NOV32j, | TMGTLPKRVKIVEVGPRDGLQNEKNIVSTPVKIKLIDMLSEAGLSVIETTSFVSPKWVPQMGDHTEV |
| CG96859-08 | |
| Protein | LKGIQKFPGINYPVLTPNLKGFEAAVAAGAKEVVIFGAASELFTKKNINCSIEESFQRFDAILKAAQ |
| Sequence | |
| | SANISVRGYVSCALGCPYEGKISPAKVAEVTKKFYSMGCYEISLGDTIGVGTPGIMKDMLSAVMQEV |
| | PLAALAVHCHDTYGQALANTLMALQMGVSVVDSSVAGLGGCPYAQGASGNLATEDLVYMLEGLGIHT |
| | GVNLQKLLEAGNFICQALNRKTSSKVAQATCKL |

| | |
|---|---|
| SEQ ID NO: 339 | 987 bp |

| | |
|---|---|
| NOV32k, | TAACTTTATTATTAAAAATTAAAGAGGTATATATTAATGTATCGATTAAATAAGGAGGAATAAACCA |
| GG96859-09 | |
| DNA Sequence | TGGGCCACCATCACCACCATCACACTTTACCAAAGCGGGTGAAAATTGTGGAAGTTGGTCCCCGAGA |
| | TGGACTACAAAATGAAAAGAATATCGTATCTACTCCAGTGAAAATCAAGCTGATAGACATGCTTTCT |
| | GAAGCAGGACTCTCTGTTATAGAAACCACCAGCTTTGTGTCTCCTAAGTGGGTTCCCCAGATGGGTG |
| | ACCACACTGAAGTCTTGAAGGGCATTCAGAAGTTTCCTGGCATCAACTACCCAGTCCTGACCCCAAA |
| | TTTGAAAGGCTTCGAGGCAGCGGTTGCTGCTGGAGCCAAGGAAGTAGTCATCTTTGGAGCTGCCTCA |
| | GAGCTCTTCACCAAGAAGAACATCAATTGTTCCATAGAGGAGAGTTTTCAGAGGTTTGACGCAATCC |
| | TGAAGGCAGCGCAGTCAGCCAATATTTCTGTGCGGGGGTACGTCTCCTGTGCTCTTGGCTGCCCTTA |
| | TGAAGGGAAGATCTCCCCAGCTAAAGTAGCTGAGGTCACCAAGAAGTTCTACTCAATGGGCTGCTAC |
| | GAGATCTCCCTGGGGGACACCATTGGTGTGGGCACCCCAGGGATCATGAAAGACATGCTGTCTGCTG |
| | TCATGCAGGAAGTGCCTCTGGCTGCCCTGGCTGTCCACTGCCATGACACCTATGGTCAAGCCCTGGC |
| | CAACACCTTGATGGCCCTGCAGATGGGAGTGAGTGTCGTGGACTCTTCTGTGGCAGGACTTGGAGGC |
| | TGTCCCTACGCACAGGGGGCATCAGGAAACTTGGCCACAGAAGACCTGGTCTACATGCTAGAGGGCT |
| | TGGGCATTCACACGGGTGTGAATCTCCAGAAGCTTCTGGAAGCTGGAAACTTTATCTGTCAAGCCCT |
| | GAACAGAAAAACTAGCTCCAAAGTGGCTCAGGCTACCTGTAAACTCTGA |

| | | | |
|---|---|---|---|
| ORF Start: at 64 | | | ORF Stop: TGA at 985 |
| SEQ ID NO: 340 | 307 aa | MW at 32658.6 kD | |

| | |
|---|---|
| NOV32k, | TMGHHHHHHTLPKRVKIVEVGPRDGLQNEKNIVSTPVKIKLIDMLSEAGLSVIETTSFVSPKWVPQM |
| CG96859-09 | |
| Protein Sequence GDHTEVLKGIQKFPGINYPVLTPNLKGFEAAVAAGAKEVVIFGAASELFTKKNINCSIEESFQRFDA | |
| | ILKAAQSANISVRGYVSCALGCPYEGKISPAKVAEVTKKFYSMGCYEISLGDTIGVGTPGIMKDMLS |
| | AVMQEVPLAALAVHCHDTYGQALANTLMALQMGVSVVDSSVAGLGGCPYAQGASGNLATEDLVYMLE |
| | GLGIHTGVNLQKLLEAGNFICQALNRKTSSKVAQATCKL |

Sequence comparison of the above protein sequences yields the following sequence relationships shown in Table 32B.

TABLE 32B
Comparison of NOV32a against NOV32b through NOV32k.

| | NOV32a Residues/ | Identities/Similarities |
|---|---|---|
| Protein Sequence | Match Residues | for the Matched Region |
| NOV32b | 25 . . . 325 | 300/301 (99%) |
| | 1 . . . 301 | 301/301 (99%) |
| NOV32c | 28 . . . 325 | 298/298 (100%) |
| | 10 . . . 307 | 298/298 (100%) |
| NOV32d | 1 . . . 325 | 324/325 (99%) |
| | 1 . . . 325 | 324/325 (99%) |
| NOV32e | 186 . . . 325 | 139/140 (99%) |
| | 115 . . . 254 | 139/140 (99%) |
| NOV32f | 186 . . . 325 | 139/140 (99%) |
| | 115 . . . 254 | 139/140 (99%) |
| NOV32g | 1 . . . 325 | 288/325 (88%) |
| | 1 . . . 289 | 288/325 (88%) |
| NOV32h | 1 . . . 323 | 323/323 (100%) |
| | 1 . . . 323 | 323/323 (100%) |
| NOV32i | 25 . . . 325 | 300/301 (99%) |
| | 1 . . . 301 | 301/301 (99%) |
| NOV32j | 25 . . . 325 | 300/301 (99%) |
| | 1 . . . 301 | 301/301 (99%) |
| NOV32k | 28 . . . 325 | 298/298 (100%) |
| | 10 . . . 307 | 298/298 (100%) |

Further analysis of the NOV32a protein yielded the following properties shown in Table 32C.

TABLE 32C
Protein Sequence Properties NOV32a

| SignalP | | |
|---|---|---|
| analysis: | Cleavage site between residues 25 and 26 | |
| PSORT II | PSG: | a new signal peptide prediction method |
| analysis: | | N-region: length 11; pos. chg 4; neg. chg 0 |
| | | H-region: length 7; peak value 0.99 |
| | | PSG score: −3.41 |
| | GvH: | von Heijne's method for signal seq. recognition |
| | | GvH score (threshold: −2.1): −2.69 |
| | | possible cleavage site: between 23 and 24 |
| | >>> Seems to have no N-terminal signal peptide | |
| | ALOM: | Klein et al's method for TM region allocation |
| | | Init position for calculation: 1 |
| | | Tentative number of TMS(s) for the threshold 0.5: 0 |
| | | number of TMS(s) . . . fixed |
| | | PERIPHERAL Likelihood = 1.80 (at 115) |
| | | ALOM score: 1.80 (number of TMSs: 0) |

MITDISC: discrimination of mitochondrial targeting seq

| | | | |
|---|---|---|---|
| R content: | 5 | Hyd Moment | 10.64 |
| Hyd Moment (95): | 9.40 | (75): | |
| D/E content: | 1 | G content: | 2 |
| Score: | 1.44 | S/T content: | 6 |

| Gavel: | prediction of cleavage sites for mitochondrial preseq |
|---|---|
| | R-2 motif at 42 KRV\|KI |
| NUCDISC: discrimination of nuclear localization signals | |
| | pat4: none |
| | pat7: PKRVKIV (5) at 30 |
| | bipartite: none |
| | content of basic residues: 9.8% |
| | NLS Score: −0.04 |
| KDEL: | ER retention motif in the C-terminus: none |
| ER Membrane Retention Signals: | |
| | XXRR-like motif in the N-terminus: AAMR |
| | KKXX-like motif in the C-terminus: ATCK |
| SKL: | peroxisomal targeting signal in the C-terminus: CKL |
| PTS2: | 2nd peroxisomal targeting signal: none |
| VAC: | possible vacuolar targeting motif: found |
| | TLPK at 28 |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: cytoplasmic |
| | Reliability: 94.1 |
| COIL: | Lupas's algorithm to detect coiled-coil regions |
| | total: 0 residues |

----------------------------------
Final Results (k = 9/23):

```
87.0%: mitochondrial
  4.3%: Golgi
  4.3%: cytoplasmic
  4.3%: nuclear
```

>> prediction for CG96859-03 is mit (k = 23)

A search of the NOV32a protein against the Geneseq database, a proprietary database that contains sequences published in patents and patent publication, yielded several homologous proteins shown in Table 32D.

TABLE 32D
Geneseq Results for NOV32a

| | | | Identities/ | |
|---|---|---|---|---|
| | | NOV32a | Similarities | |
| | Protein/Organism/ | Residues/ | for the | |
| Geneseq | Length | Match | Matched | Expect |
| Identifier | [Patent #, Date] | Residues | Region | Value |
| AAU75774 | Human 3-hydroxy- | 1 . . . 325 | 324/325 | 0.0 |
| | 3-methylglutaryl | | (99%) | |
| | coenzyme A lyase | 1 . . . 325 | 324/325 | |
| | (HMGCL) protein - | | (99%) | |
| | Homo sapiens, | | | |
| | 325 aa. | | | |
| | [WO200198315-A2, | | | |
| | 27-DEC-2001] | | | |
| AAU01613 | Gene #24 human | 30 . . . 321 | 234/292 | e−138 |
| | secreted protein | | (80%) | |
| | homologous amino | 1 . . . 292 | 266/292 | |
| | acid sequence - | | (90%) | |
| | Homo sapiens, | | | |
| | 293 aa. | | | |
| | [WO200123547-A1, | | | |
| | 05-APR-2001] | | | |
| AAU01614 | Human secreted | 30 . . . 322 | 212/293 | e−125 |
| | protein encoded by | | (72%) | |
| | gene #24 - Homo | 1 . . . 293 | 254/293 | |
| | sapiens, 293 aa. | | (86%) | |
| | [WO200123547-A1, | | | |
| | 05-APR-2001] | | | |
| AAE19936 | Soybean HMG-CoA | 26 . . . 321 | 195/296 | e−108 |
| | lyase #1 - Glycine | | (65%) | |
| | max, 310 aa. | 15 . . . 310 | 231/296 | |
| | [US6348339-B1, | | (77%) | |
| | 19-FEB-2002] | | | |
| AAE19935 | Rice HMG-CoA | 29 . . . 321 | 192/293 | e−108 |
| | lyase #2 - Oryza | | (65%) | |
| | sativa, 459 aa. | 154 . . . 446 | 231/293 | |
| | [US6348339-B1, | | (78%) | |
| | 19-FEB-2002] | | | |

In a BLAST search of public sequence datbases, the NOV32a protein was found to have homology to the proteins shown in the BLASTP data in Table 32E.

TABLE 32E
Public BLASTP Results for NOV32a

| | | | Identities/ | |
|---|---|---|---|---|
| | | NOV32a | Similarities | |
| Protein | | Residues/ | for the | |
| Accession | Protein/Organism/ | Match | Matched | Expect |
| Number | Length | Residues | Portion | Value |
| P35914 | Hydroxymethyl- | 1 . . . 325 | 325/325 | 0.0 |
| | glutaryl-CoA lyase, | | (100%) | |
| | mitochondrial | 1 . . . 325 | 325/325 | |
| | precursor | | (100%) | |
| | (EC 4.1.3.4) | | | |
| | (HMG-CoA lyase) | | | |
| | (HL) (3-hydroxy-3- | | | |
| | methylglutarate- | | | |
| | CoA lyase) - | | | |
| | Homo sapiens | | | |
| | (Human), 325 aa. | | | |
| A45470 | hydroxymethyl- | 1 . . . 325 | 324/325 | 0.0 |
| | glutaryl-CoA lyase | | (99%) | |
| | (EC 4.1.3.4) - | 1 . . . 325 | 324/325 | |
| | human, 325 aa. | | (99%) | |
| BAC20595 | 3-hydroxymethyl-3- | 1 . . . 325 | 313/325 | e−176 |
| | methylglutaryl- | | (96%) | |
| | Coenzyme A lyase - | 1 . . . 325 | 315/325 | |
| | Macaca fascicularis | | (96%) | |
| | (Crab eating | | | |
| | macaque) | | | |
| | (Cynomolgus | | | |
| | monkey), 325 aa. | | | |
| Q96TG6 | DJ886K2.2 | 21 . . . 325 | 305/305 | e−172 |
| | (EC 4.1.3.4) | | (100%) | |
| | (HMGCL(hydroxy- | 1 . . . 305 | 305/305 | |
| | methylglutaryl-CoA | | (100%) | |
| | lyase)) (HMG-CoA | | | |
| | lyase) (HL) | | | |
| | (3-hydroxy-3- | | | |
| | methylglutarate- | | | |
| | CoA lyase) - Homo | | | |
| | sapiens (Human), | | | |
| | 305 aa (fragment). | | | |
| P97519 | Hydroxymethyl- | 1 . . . 325 | 289/325 | e−167 |
| | glutaryl-CoA lyase, | | (88%) | |
| | mitochondrial | 1 . . . 325 | 311/325 | |
| | precursor | | (94%) | |
| | (EC 4.1.3.4) | | | |
| | (HMG-CoA lyase) | | | |
| | (HL) (3-hydroxy-3- | | | |
| | methylglutarate- | | | |
| | CoA lyase) - | | | |
| | Rattus norvegicus | | | |
| | (Rat), 325 aa. | | | |

PFam analysis predicts that the NOV32a protein contains the domains shown in the Table 32F.

TABLE 32F
Domain Analysis of NOV32a

| | NOV32a Match | Identities/Similarities | Expect |
|---|---|---|---|
| Pfam Domain | Region | for the Matched Region | Value |
| HMGL-like | 41 . . . 318 | 103/307 (34%) | 2e−118 |
| | | 250/307 (81%) | |

### Example 33

The NOV33 clone was analyzed, and the nucleotide and encoded polypeptide sequences are shown in Table 33A.

TABLE 33A
NOV33 Sequence Analysis

| | |
|---|---|
| SEQ ID NO: 341 | 2551 bp |

| | |
|---|---|
| NOV33a, | CACCATGAACAGCAGCAGCGCCAACATCACCTACGCCAGTCGCAAGCGGCGGAAGCCGGTGCAGAAAA |
| CG105355-03 | |
| DNA Sequence | CAGTAAAGCCAATCCCAGCTGAAGGAATCAAGTCAAATCCTTCCAAGCGGCATAGAGACCGACTTAAT |
| | ACAGAGTTGGACCGTTTGGCTAGCCTGCTGCCTTTCCCACAAGATGTTATTAATAAGTTGGACAAACT |
| | TTCAGTTCTTAGGCTCAGCGTCAGTTACCTGAGAGCCAAGAGCTTCTTTGATGTTGCATTAAAATCCT |
| | CCCCTACTGAAAGAAACGGAGGCCAGGATAACTGTAGAGCAGCAAATTTCAGAGAAGGCCTGAACTTA |
| | CAAGAAGGAGAATTCTTATTACAGGCTCTGAATGGCTTTGTATTAGTTGTCACTACAGATGCTTTGGT |
| | CTTTTATGCTTCTTCTACTATACAAGATTATCTAGGGTTTCAGCAGTCTGATGTCATACATCAGAGTG |
| | TATATGAACTTATCCATACCGAAGACCGAGCTGAATTTCAGCGTCAGCTACACTGGGCATTAAATCCT |
| | TCTCAGTGTACAGAGTCTGGACAAGGAATTGAAGAAGCCACTGGTCTCCCCCAGACAGTAGTCTGTTA |
| | TAACCCAGACCAGATTCCTCCAGAAAACTCTCCTTTAATGGAGAGGTGCTTCATATGTCGTCTAAGGT |
| | GTCTGCTGGATAATTCATCTGGTTTTCTGGCAATGAATTTCCAAGGGAAGTTAAAGTATCTTCATGGA |
| | CAGAAAAAGAAAGGGAAAGATGGATCAATACTTCCACCTCAGTTGGCTTTGTTTGCGATAGCTACTCC |
| | ACTTCAGCCACCATCCATACTTGAAATCCGGACCAAAAATTTTATCTTTAGAACCAAACACAAACTAG |
| | ACTTCACACCTATTGGTTGTGATGCCAAAGGAAGAATTGTTTTAGGATATACTGAAGCAGAGCTGTGC |
| | ACGAGAGGCTCAGGTTATCAGTTTATTCATGCAGCTGATATGCTTTATTGTGCCGAGTCCCATATCCG |
| | AATGATTAAGACTGGAGAAAGTGGCATGATAGTTTTCCGGCTTCTTACAAAAAACAACCGATGGACTT |
| | GGGTCCAGTCTAATGCACGCCTGCTTTATAAAAATGGAAGACCAGATTATATCATTGTAACTCAGAGA |
| | CCACTAACAGATGAGGAAGGAACAGAGCATTTACGAAAACGAAATACGAAGTTGCCTTTTATGTTTAC |
| | CACTGGAGAAGCTGTGTTGTATGAGGCAACCAACCCTTTTCCTGCCATAATGGATCCCTTACCACTAA |
| | GGACTAAAAATGGCACTAGTGGAAAAGACTCTGCTACCACATCCACTCTAAGCAAGGACTCTCTCAAT |
| | CCTAGTTCCCTCCTGGCTGCCATGATGCAACAAGATGAGTCTATTTATCTCTATCCTGCTTCAAGTAC |
| | TTCAAGTACTGCACCTTTTGAAAACAACTTTTTCAACGAATCTATGAATGAATGCAGAAATTGGCAAG |
| | ATAATACTGCACCGATGGGAAATGATACTATCCTGAAACATGAGCAAATTGACCAGCCTCAGGATGTG |
| | AACTCATTTGCTGGAGGTCACCCAGGGCTCTTTCAAGATAGTAAAAACAGTGACTTGTACAGCATAAT |
| | GAAAAACCTAGGCATTGATTTTGAAGACATCAGACACATGCAGAATGAAAAATTTTTCAGAAATGATT |
| | TTTCTGGTGAGGTTGACTTCAGAGACATTGACTTAACGGATGAAATCCTGACGTATGTCCAAGATTCT |
| | TTAAGTAAGTCTCCCTTCATACCTTCAGATTATCAACAGCAACAGTCCTTGGCTCTGAACTCAAGCTG |
| | TATGGTACAGGAACACCTACATCTAGAACAGCAACAGCAACATCACCAAAAGCAAGTAGTAGTGGAGC |
| | CACAGCAACAGCTGTGTCAGAAGATGAAGCACATGCAAGTTAATGGCATGTTTGAAAATTGGAACTCT |
| | AACCAATTCGTGCCTTTCAATTGTCCACAGCAAGACCCACAACAATATAATGTCTTTACAGACTTACA |
| | TGGGATCAGTCAAGAGTTCCCCTACAAATCTGAAATGGATTCTATGCCTTATACACAGAACTTTATTT |
| | CCTGTAATCAGCCTGTATTACCACAACATTCCAAATGTACAGAGCTGGACTACCCTATGGGGAGTTTT |
| | GAACCATCCCCATACCCCACTACTTCTAGTTTAGAAGATTTTGTCACTTGTTTACAACTTCCTGAAAA |
| | CCAAAAGCATGGATTAAATCCACAGTCAGCCATAATAACTCCTCAGACATGTTATGCTGGGGCCGTGT |
| | CGATGTATCAGTGCCAGCCAGAACCTCAGCACACCCACGTGGGTCAGATGCAGTACAATCCAGTACTG |
| | CCAGGCCAACAGGCATTTTTAAACAAGTTTCAGAATGGAGTTTTAAATGAAACATATCCAGCTGAATT |
| | AAATAACATAAATAACACTCAGACTACCACACATCTTCAGCCACTTCATCATCCGTCAGAAGCCAGAC |
| | CTTTTCCTGATTTGACATCCAGTGGATTCCTGTAA |

| | | |
|---|---|---|
| ORF Start: at 2 | | ORF Stop: TAA at 2549 |
| SEQ ID NO: 342 | 849 aa | MW at 96247.6 kD |

| | |
|---|---|
| NOV33a, | TMNSSSANITYASRKRRKPVQKTVKPIPAEGIKSNPSKRHRDRLNTELDRLASLLPFPQDVINKLDKL |
| CG105355-03 | |
| Protein | SVLRLSVSYLRAKSFFDVALKSSPTERNGGQDNCRAANFREGLNLQEGEFLLQALNGFVLVVTTDALV |
| Sequence | |
| | FYASSTIQDYLGFQQSDVIHQSVYELIHTEDRAEFQRQLHWALNPSQCTESGQGIEEATGLPQTVVCY |
| | NPDQIPPENSPLMERCFICRLRCLLDNSSGFLAMNFQGKLKYLHGQKKKGKDGSILPPQLALFAIATP |
| | LQPPSILEIRTKNFIFRTKHKLDFTPIGCDAKGRIVLGYTEAELCTRGSGYQFIHAADMLYCAESHIR |
| | MIKTGESGMIVFRLLTKNNRWTWVQSNARLLYKNGRPDYIIVTQRPLTDEEGTEHLRKRNTKLPFMFT |
| | TGEAVLYEATNPFPAIMDPLPLRTKNGTSGKDSATTSTLSKDSLNPSSLLAAMMQQDESIYLYPASST |
| | SSTAPFENNFFNESMNECRNWQDNTAPMGNDTILKHEQIDQPQDVNSFAGGHPGLFQDSKNSDLYSIM |
| | KNLGIDFEDIRHMQNEKFFRNDFSGEVDFRDIDLTDEILTYVQDSLSKSPFIPSDYQQQQSLALNSSC |
| | MVQEHLHLEQQQQHHQKQVVVEPQQQLCQKMKHMQVNGMFENWNSNQFVPFNCPQQDPQQYNVFTDLH |
| | GISQEFPYKSEMDSMPYTQNFISCNQPVLPQHSKCTELDYPMGSFEPSPYPTTSSLEDFVTCLQLPEN |
| | QKHGLNPQSAIITPQTCYAGAVSMYQCQPEPQHTHVGQMQYNPVLPGQQAFLNKFQNGVLNETYPAEL |
| | NNINNTQTTTHLQPLHHPSEARPFPDLTSSGFL |

| | |
|---|---|
| SEQ ID NO: 343 | 5864 bp |

| | |
|---|---|
| NOV33b, | CAGTGGCTGGGGAGTCCCGTCGACGCTCTGTTCCGAGAGCGTGCCCCGGACCGCCAGCTCAGAACAGG |
| CG105355-01 | |
| DNA Sequence | GGCAGCCGTGTAGCCGAACGGAAGCTGGGAGCAGCCGGGACTGGTGGCCCGCGCCCGAGCTCCGCAGG |
| | CGGGAAGCACCCTGGATTTGGGAAGTCCCGGGAGCAGCGCGGCGGCACCTCCCTCACCCAAGGGGCCG |
| | CGGCGACGGTCACGGGGCGCGGCGCCACCGTGAGCGACCCAGGCCAGGATTCTAAATAGACGGCCCAG |
| | GCTCCTCCTCCGCCCGGGCCGCCTCACCTGCGGGCATTGCCGCGCCGCCTCCGCCGGTGTAGACGGCA |
| | CCTGCGCCGCCTTGCTCGCGGGTCTCCGCCCCTCGCCCACCCTCACTGCGCCAGGCCCAGGCAGCTCA |
| | CCTGTACTGGCGCGGGCTGCGGAAGCCTGCGTGAGCCGAGGCGTTGAGGCGCGGCGCCCACGCCACTG |
| | TCCCGAGAGGACGCAGGTGGAGCGGGCGCGGCTTCGCGGAACCCGGCGCCGGCCGCCGCAGTGGTCCC |
| | AGCCTACACCGGGTTCCGGGGACCCGGCCGCCAGTGCCCGGGGAGTAGCCGCCGCCGTCGGCTGGGCA |
| | CCATGAACAGCAGCAGCGCCAACATCACCTACGCCAGTCGCAAGCGGCGGAAGCCGGTGCAGAAAACA |
| | GTAAAGCCAATCCCAGCTGAAGGAATCAAGTCAAATCCTTCCAAGCGGCATAGAGACCGACTTAATAC |
| | AGAGTTGGACCGTTTGGCTAGCCTGCTGCCTTTCCCACAAGATGTTATTAATAAGTTGGACAAACTTT |
| | CAGTTCTTAGGCTCAGCGTCAGTTACCTGAGAGCCAAGAGCTTCTTTGATGTTGCATTAAAATCCTCC |
| | CCTACTGAAAGAAACGGAGGCCAGGATAACTGTAGAGCAGCAAATTTCAGAGAAGGCCTGAACTTACA |
| | AGAAGGAGAATTCTTATTACAGGCTCTGAATGGCTTTGTATTAGTTGTCACTACAGATGCTTTGGTCT |
| | TTTATGCTTCTTCTACTATACAAGATTATCTAGGGTTTCAGCAGTCTGATGTCATACATCAGAGTGTA |
| | TATGAACTTATCCATACCGAAGACCGAGCTGAATTTCAGCGTCAGCTACACTGGGCATTAAATCCTTC |
| | TCAGTGTACAGAGTCTGGACAAGGAATTGAAGAAGCCACTGGTCTCCCCCAGACAGTAGTCTGTTATA |
| | ACCCAGACCAGATTCCTCCAGAAAACTCTCCTTTAATGGAGAGGTGCTTCATATGTCGTCTAAGGTGT |
| | CTGCTGGATAATTCATCTGGTTTTCTGGCAATGAATTTCCAAGGGAAGTTAAAGTATCTTCATGGACA |
| | GAAAAAGAAAGGGAAAGATGGATCAATACTTCCACCTCAGTTGGCTTTGTTTGCGATAGCTACTCCAC |
| | TTCAGCCACCATCCATACTTGAAATCCGGACCAAAAATTTTATCTTTAGAACCAAACACAAACTAGAC |
| | TTCACACCTATTGGTTGTGATGCCAAAGGAAGAATTGTTTTAGGATATACTGAAGCAGAGCTGTGCAC |
| | GAGAGGCTCAGGTTATCAGTTTATTCATGCAGCTGATATGCTTTATTGTGCCGAGTCCCATATCCGAA |
| | TGATTAAGACTGGAGAAAGTGGCATGATAGTTTTCCGGCTTCTTACAAAAAACAACCGATGGACTTGG |
| | GTCCAGTCTAATGCACGCCTGCTTTATAAAAATGGAAGACCAGATTATATCATTGTAACTCAGAGACC |
| | ACTAACAGATGAGGAAGGAACAGAGCATTTACGAAAACGAAATACGAAGTTGCCTTTTATGTTTACCA |
| | CTGGAGAAGCTGTGTTGTATGAGGCAACCAACCCTTTTCCTGCCATAATGGATCCCTTACCACTAAGG |
| | ACTAAAAATGGCACTAGTGGAAAAGACTCTGCTACCACATCCACTCTAAGCAAGGACTCTCTCAATCC |
| | TAGTTCCCTCCTGGCTGCCATGATGCAACAAGATGAGTCTATTTATCTCTATCCTGCTTCAAGTACTT |
| | CAAGTACTGCACCTTTTGAAAACAACTTTTTCAACGAATCTATGAATGAATGCAGAAATTGGCAAGAT |
| | AATACTGCACCGATGGGAAATGATACTATCCTGAAACATGAGCAAATTGACCAGCCTCAGGATGTGAA |
| | CTCATTTGCTGGAGGTCACCCAGGGCTCTTTCAAGATAGTAAAAACAGTGACTTGTACAGCATAATGA |
| | AAAACCTAGGCATTGATTTTGAAGACATCAGACACATGCAGAATGAAAAATTTTTCAGAAATGATTTT |
| | TCTGGTGAGGTTGACTTCAGAGACATTGACTTAACGGATGAAATCCTGACGTATGTCCAAGATTCTTT |
| | AAGTAAGTCTCCCTTCATACCTTCAGATTATCAACAGCAACAGTCCTTGGCTCTGAACTCAAGCTGTA |
| | TGGTACAGGAACACCTACATCTAGAACAGCAACAGCAACATCACCAAAAGCAAGTAGTAGTGGAGCCA |
| | CAGCAACAGCTGTGTCAGAAGATGAAGCACATGCAAGTTAATGGCATGTTTGAAAATTGGAACTCTAA |
| | CCAATTCGTGCCTTTCAATTGTCCACAGCAAGACCCACAACAATATAATGTCTTTACAGACTTACATG |
| | GGATCAGTCAAGAGTTCCCCTACAAATCTGAAATGGATTCTATGCCTTATACACAGAACTTTATTTCC |
| | TGTAATCAGCCTGTATTACCACAACATTCCAAATGTACAGAGCTGGACTACCCTATGGGGAGTTTTGA |
| | ACCATCCCCATACCCCACTACTTCTAGTTTAGAAGATTTTGTCACTTGTTTACAACTTCCTGAAAACC |
| | AAAAGCATGGATTAAATCCACAGTCAGCCATAATAACTCCTCAGACATGTTATGCTGGGGCCGTGTCG |
| | ATGTATCAGTGCCAGCCAGAACCTCAGCACACCCACGTGGGTCAGATGCAGTACAATCCAGTACTGCC |
| | AGGCCAACAGGCATTTTTAAACAAGTTTCAGAATGGAGTTTTAAATGAAACATATCCAGCTGAATTAA |
| | ATAACATAAATAACACTCAGACTACCACACATCTTCAGCCACTTCATCATCCGTCAGAAGCCAGACCT |
| | TTTCCTGATTTGACATCCAGTGGATTCCTGTAATTCCAAGCCCAATTTTGACCCTGGTTTTTGGATTA |
| | AATTAGTTTGTGAAGGATTATGGAAAAATAAAACTGTCACTGTTGGACGTCAGCAAGTTCACATGGAG |
| | GCATTGATGCATGCTATTCACAATTATTCCAAACCAAATTTTAATTTTTGCTTTTAGAAAAGGGAGTT |
| | TAAAAATGGTATCAAAATTACATATACTACAGTCAAGATAGAAAGGGTGCTGCCACGGAGTGGTGAGG |
| | TACCGTCTACATTTCACATTATTCTGGGCACCACAAAATATACAAAACTTTATCAGGGAAACTAAGAT |
| | TCTTTTAAATTAGAAAATATTCTCTATTTGAATTATTTCTGTCACAGTAAAAATAAAATACTTTGAGT |
| | TTTGAGCTACTGGATTCTTATTAGTTCCCCAAATACAAAGTTAGAGAACTAAACTAGTTTTTCCTATC |
| | ATGTTAACCTCTGCTTTTATCTCAGATGTTAAAATAAATGGTTTGGTGCTTTTTATAAAAAGATAATC |
| | TCAGTGCTTTCCTCCTTCACTGTTTCATCTAAGTGCCTCACATTTTTTTCTACCTATAACACTCTAGG |
| | ATGTATATTTTATATAAAGTATTCTTTTTCTTTTTTAAATTAATATCTTTCTGCACACAAATATTATT |
| | TGTGTTTCCTAAATCCAACCATTTTCATTAATTCAGGCATATTTTAACTCCACTGCTTACCTACTTTC |
| | TTCAGGTAAAGGGCAAATAATGATCGAAAAAATAATTATTTATTACATAATTTAGTTGTTTCTAGACT |
| | ATAAATGTTGCTATGTGCCTTATGTTGAAAAAATTTAAAAGTAAAATGTCTTTCCAAATTATTTCTTA |
| | ATTATTATAAAAATATTAAGACAATAGCACTTAAATTCCTCAACAGTGTTTTCAGAAGAAATAAATAT |
| | ACCACTCTTTACCTTTATTGATATCTCCATGATGATAGTTGAATGTTGCAATGTGAAAAATCTGCTGT |
| | TAACTGCAACCTTGTGTATTAAATTGCAAGAAGCTTTATTTCTAGCTTTTTAATTAAGCAAAGCACCC |
| | ATTTCAATGTGTATAAATTGTCTTTAAAAACTGTTTTAGACCTATAATCCTTGATAATATATTGTGTT |
| | GACTTTATAAATTTCGCTTCTTAGAACAGTGGAAACTATGTGTTTTTCTCATATTTGAGGAGTGTTAA |
| | GATTGCAGATAGCAAGGTTTGGTGCAAAGTATTGTAATGAGTGAATTGAATGGTGCATTGTATAGATA |
| | TAATGAACAAAATTATTTGTAAGATATTTGCAGTTTTTCATTTTAAAAAGTCCATACCTTATATATGC |
| | ACTTAATTTGTTGGGGCTTTACATACTTTATCAATGTGTCTTTCTAAGAAATCAAGTAATGAATCCAA |
| | CTGCTTAAAGTTGGTATTAATAAAAAGACAACCACATAGTTCGTTTACCTTCAAACTTTAGGTTTTTT |
| | TAATGATATACTGATCTTCATTACCAATAGGCAAATTAATCACCCTACCAACTTTACTGTCCTAACAT |
| | GGTTTAAAAGAAAAAATGACACCATCTTTTATTCTTTTTTTTTTTTTTTTTGAGAGAGAGTCTTACTC |
| | TGCCGCCCAAACTGGAGTGCAGTGGCACAATCTTGGCTCACTGCAACCTCTACCTCCTGGGTTCAAGT |
| | GATTCTCTTGCCTCAGCCTCCCGAGTTGCTGGGATTGCGGGCATGGTGGCGTGAGCCTGTAGTCCTAG |
| | CTACTCGGGAGGCTGAGGCAGGAGAATAGCCTGAACCTGGGAATCGGAGGTTGCAGGGCCAAGATCGC |
| | CCCACTGCACTCCAGCCTGGCAATAGACCGAGACTCCGTCTCCAAAAAAAAAAAAAATACAATTTTTA |
| | TTTCTTTTACTTTTTTTAGTAAGTTAATGTATATAAAAATGGCTTCGGACAAAATATCTCTGAGTTCT |
| | GTGTATTTTCAGTCAAAACTTTAAACCTGTAGAATCAATTTAAGTGTTGGAAAAAATTTGTCTGAAAC |
| | ATTTCATAATTTGTTTCCAGCATGAGGTATCTAAGGATTTAGACCAGAGGTCTAGATTAATACTCTAT |
| | TTTTACATTTAAACCTTTTATTATAAGTCTTACATAAACCATTTTTGTTACTCTCTTCCACATGTTAC |
| | TGGATAAATTGTTTAGTGGAAAATAGGCTTTTTAATCATGAATATGATGACAATCAGTTATACAGTTA |
| | TAAAATTAAAAGTTTGAAAAGCAATATTGTATATTTTTATCTATATAAAATAACTAAAATGTATCTAA |
| | GAATAATAAAATCACGTTAAACCAAATACACGTTTGTCTGTATTGTTAAGTGCCAAACAAAGGATACT |
| | TAGTGCACTGCTACATTGTGGGATTTATTTCTAGATGATGTGCACATCTAAGGATATGGATGTGTCTA |
| | ATTTTAGTCTTTTCCTGTACCAGGTTTTTCTTACAATACCTGAAGACTTACCAGTATTCTAGTGTATT |
| | ATGAAGCTTTCAACATTACTATGCACAAACTAGTGTTTTTCGATGTTACTAAATTTTAGGTAAATGCT |
| | TTCATGGCTTTTTTCTTCAAAATGTTACTGCTTACATATATCATGCATAGATTTTTGCTTAAAGTATG |
| | ATTTATAATATCCTCATTATCAAAGTTGTATACAATAATATATAATAAAATAACAAATATGAATAATA |
| | AAAAAAAAAAAAAAAA |

| | | |
|---|---|---|
| ORF Start: ATG at 615 | | ORF Stop: TAA at 3159 |
| SEQ ID NO: 344 | 848 aa | MW at 96146.5 kD |

| | |
|---|---|
| NOV33b, | MNSSSANITYASRKRRKPVQKTVKPIPAEGIKSNPSKRHRDRLNTELDRLASLLPFPQDVINKLDLKS |
| CG105355-01 | |
| Protein | VLRLSVSYLRAKSFFDVALKSSPTERNGGQDNCRAANFREGLNLQEGEFLLQALNGFVLVVTTDALVF |
| Sequence | |
| | YASSTIQDYLGFQQSDVIHQSVYELIHTEDRAEFQRQLHWALNPSQCTESGQGIEEATGLPQTVVCYN |
| | PDQIPPENSPLMERCFICRLRCLLDNSSGFLAMNFQGKLKYLHGQKKKGKDGSILPPQLALFAIATPL |
| | QPPSILEIRTKNFIFRTKHKLDFTPIGCDAKGRIVLGYTEAELCTRGSGYQFIHAADMLYCAESHIRM |
| | IKTGESGMIVFRLLTKNNRWTWVQSNARLLYKNGRPDYIIVTQRPLTDEEGTEHLRKRNTKLPFMFTT |
| | GEAVLYEATNPFPAIMDPLPLRTKNGTSGKDSATTSTLSKDSLNPSSLLAAMMQQDESIYLYPASSTS |
| | STAPFENNFFNESMNECRNWQDNTAPMGNDTILKHEQIDQPQDVNSFAGGHPGLFQDSKNSDLYSIMK |
| | NLGIDFEDIRHMQNEKFFRNDFSGEVDFRDIDLTDEILTYVQDSLSKSPFIPSDYQQQQSLALNSSCM |
| | VQEHLHLEQQQQHHQKQVVVEPQQQLCQKMKHMQVNGMFENWNSNQFVPFNCPQQDPQQYNVFTDLHG |
| | ISQEFPYKSEMDSMPYTQNFISCNQPVLPQHSKCTELDYPMGSFEPSPYPTTSSLEDFVTCLQLPENQ |
| | KHGLNPQSAIITPQTCYAGAVSMYQCQPEPQHTHVGQMQYNPVLPGQQAFLNKFQNGVLNETYPAELN |
| | NINNTQTTTHLQPLHHPSEARPFPDLTSSGFL |

| | |
|---|---|
| SEQ ID NO: 345 | 2677 bp |

| | |
|---|---|
| NOV33c, | CCAGTGCCCGGGGAGTAGCCGCCGCCGTCGGCTGGGCACCATGAACAGCAGCAGCGCCAACATCACCT |
| CG105355-02 | |
| DNA Sequence | ACGCCAGTCGCAAGCGGCGGAAGCCGGTGCAGAAAACAGTAAAGCCAATCCCAGCTGAAGGAATCAAG |
| | TCAAATCCTTCCAAGCGGCATAGAGACCGACTTAATACAGAGTTGGACCGTTTGGCTAGCCTGCTGCC |
| | TTTCCCACAAGATGTTATTAATAAGTTGGACAAACTTTCAGTTCTTAGGCTCAGCGTCAGTTACCTGA |
| | GAGCCAAGAGCTTCTTTGATGTTGCATTAAAATCCTCCCCTACTGAAAGAAACGGAGGCCAGGATAAC |
| | TGTAGAGCAGCAAATTTCAGAGAAGGCCTGAACTTACAAGAAGGAGAATTCTTATTACAGGCTCTGAA |
| | TGGCTTTGTATTAGTTGTCACTACAGATGCTTTGGTCTTTTATGCTTCTTCTACTATACAAGATTATC |
| | TAGGGTTTCAGCAGTCTGATGTCATACATCAGAGTGTATATGAACTTATCCATACCGAAGACCGAGCT |
| | GAATTTCAGCGTCAGCTACACTGGGCATTAAATCCTTCTCAGTGTACAGAGTCTGGACAAGGAATTGA |
| | AGAAGCCACTGGTCTCCCCCAGACAGTAGTCTGTTATAACCCAGACCAGATTCCTCCAGAAAACTCTC |
| | CTTTAATGGAGAGGTGCTTCATATGTCGTCTAAGGTGTCTGCTGGATAATTCATCTGGTTTTCTGGCA |
| | ATGAATTTCCAAGGGAAGTTAAAGTATCTTCATGGACAGAAAAAGAAAGGGAAAGATGGATCAATACT |
| | TCCACCTCAGTTGGCTTTGTTTGCGATAGCTACTCCACTTCAGCCACCATCCATACTTGAAATCCGGA |
| | CCAAAAATTTTATCTTTAGAACCAAACACAAACTAGACTTCACACCTATTGGTTGTGATGCCAAAGGA |
| | AGAATTGTTTTAGGATATACTGAAGCAGAGCTGTGCACGAGAGGCTCAGGTTATCAGTTTATTCATGC |
| | AGCTGATATGCTTTATTGTGCCGAGTCCCATATCCGAATGATTAAGACTGGAGAAAGTGGCATGATAG |
| | TTTTCCGGCTTCTTACAAAAAACAACCGATGGACTTGGGTCCAGTCTAATGCACGCCTGCTTTATAAA |
| | AATGGAAGACCAGATTATATCATTGTAACTCAGAGACCACTAACAGATGAGGAAGGAACAGAGCATTT |
| | ACGAAAACGAAATACGAAGTTGCCTTTTATGTTTACCACTGGAGAAGCTGTGTTGTATGAGGCAACCA |
| | ACCCTTTTCCTGCCATAATGGATCCCTTACCACTAAGGACTAAAAATGGCACTAGTGGAAAAGACTCT |
| | GCTACCACATCCACTCTAAGCAAGGACTCTCTCAATCCTAGTTCCCTCCTGGCTGCCATGATGCAACA |
| | AGATGAGTCTATTTATCTCTATCCTGCTTCAAGTACTTCAAGTACTGCACCTTTTGAAAACAACTTTT |
| | TCAACGAATCTATGAATGAATGCAGAAATTGGCAAGATAATACTGCACCGATGGGAAATGATACTATC |
| | CTGAAACATGAGCAAATTGACCAGCCTCAGGATGTGAACTCATTTGCTGGAGGTCACCCAGGGCTCTT |
| | TCAAGATAGTAAAAACAGTGACTTGTACAGCATAATGAAAAACCTAGGCATTGATTTTGAAGACATCA |
| | GACACATGCAGAATGAAAAATTTTTCAGAAATGATTTTTCTGGTGAGGTTGACTTCAGAGACATTGAC |
| | TTAACGGATGAAATCCTGACGTATGTCCAAGATTCTTTAAGTAAGTCTCCCTTCATACCTTCAGATTA |
| | TCAACAGCAACAGTCCTTGGCTCTGAACTCAAGCTGTATGGTACAGGAACACCTACATCTAGAACAGC |
| | AACAGCAACATCACCAAAAGCAAGTAGTAGTGGAGCCACAGCAACAGCTGTGTCAGAAGATGAAGCAC |
| | ATGCAAGTTAATGGCATGTTTGAAAATTGGAACTCTAACCAATTCGTGCCTTTCAATTGTCCACAGCA |
| | AGACCCACAACAATATAATGTCTTTACAGACTTACATGGGATCAGTCAAGAGTTCCCCTACAAATCTG |
| | AAATGGATTCTATGCCTTATACACAGAACTTTATTTCCTGTAATCAGCCTGTATTACCACAACATTCC |
| | AAATGTACAGAGCTGGACTACCCTATGGGGAGTTTTGAACCATCCCCATACCCCACTACTTCTAGTTT |
| | AGAAGATTTTGTCACTTGTTTACAACTTCCTGAAAACCAAAAGCATGGATTAAATCCACAGTCAGCCA |
| | TAATAACTCCTCAGACATGTTATGCTGGGGCCGTGTCGATGTATCAGTGCCAGCCAGAACCTCAGCAC |
| | ACCCACGTGGGTCAGATGCAGTACAATCCAGTACTGCCAGGCCAACAGGCATTTTTAAACAAGTTTCA |
| | GAATGGAGTTTTAAATGAAACATATCCAGCTGAATTAAATAACATAAATAACACTCAGACTACCACAC |
| | ATCTTCAGCCACTTCATCATCCGTCAGAAGCCAGACCTTTTCCTGATTTGACATCCAGTGGATTCCTG |
| | TAATTCCAAGCCCAATTTTGAGCCTGGTTTTTGGATTAAATTAGTTTGTGAAGGATTATGGAAAAATA |
| | AAACTGTCACTGTTGGACGTCAGCA |

| | | |
|---|---|---|
| Start: ATG at 41 | | ORF Stop: TAA at 2585 |
| SEQ ID NO: 346 | 848 aa | MW at 96146.5 kD |

| | |
|---|---|
| NOV33c, | MNSSSANITYASRKRRKPVQKTVKPIPAEGIKSNPSKRHRDRLNTELDRLASLLPFPQDVINKLDKLS |
| CG105355-02 | |
| Protein | VLRLSVSYLRAKSFFDVALKSSPTERNGGQDNCRAANFREGLNLQEGEFLLQALNGFVLVVTTDALVF |
| Sequence | |
| | YASSTIQDYLGFQQSDVIHQSVYELIHTEDRAEFQRQLHWALNPSQCTESGQGIEEATGLPQTVVCYN |
| | PDQIPPENSPLMERCFICRLRCLLDNSSGFLAMNFQGKLKYLHGQKKKGKDGSILPPQLALFAIATPL |
| | QPPSILEIRTKNFIFRTKHKLDFTPIGCDAKGRIVLGYTEAELCTRGSGYQFIHAADMLYCAESHIRM |
| | IKTGESGMIVFRLLTKNNRWTWVQSNARLLYKNGRPDYIIVTQRPLTDEEGTEHLRKRNTKLPFMFTT |
| | GEAVLYEATNPFPAIMDPLPLRTKNGTSGKDSATTSTLSKDSLNPSSLLAAMMQQDESIYLYPASSTS |
| | STAPFENNFENESMNECRNWQDNTAPMGNDTILKHEQIDQPQDVNSFAGGHPGLFQDSKNSDLYSIMK |
| | NLGIDFEDIRHMQNEKFFRNDFSGEVDFRDIDLTDEILTYVQDSLSKSPFIPSDYQQQQSLALNSSCM |
| | VQEHLHLEQQQQHHQKQVVVEPQQQLCQKMKHMQVNGMFENWNSNQFVPFNCPQQDPQQYNVFTDLHG |
| | ISQEFPYKSEMDSMPYTQNFISCNQPVLPQHSKCTELDYPMGSFEPSPYPTTSSLEDFVTCLQLPENQ |
| | KHGLNPQSAIITPQTCYAGAVSMYQCQPEPQHTHVGQMQYNPVLPGQQAFLNKFQNGVLNETYPAELN |
| | NINNTQTTTHLQPLHHPSEARPFPDLTSSGFL |

| | |
|---|---|
| SEQ ID NO: 347 | 579 bp |

| | |
|---|---|
| NOV33d, | ATGAATTTCCAAGGGAAGTTAAAGTATCTTCATGGACAGAAAAAGAAAGGGAAAGATGGATCAATACT |
| CG105355-04 | |
| DNA Sequence | TCCACCTCAGTTGGCTTTGTTTGCGATAGCTACTCCACTTCAGCCACCATCCATACTTGAAATCCGGA |
| | CCAAAAATTTTATCTTTAGAACCAAACACAAACTAGACTTCACACCTATTGGTTGTGATGCCAAAGGA |
| | AGAATTGTTTTAGGATATACTGAAGCAGAGCTGTGCACGAGAGGCTCAGGTTATCAGTTTATTTATGC |
| | AGCTGATATGCTTTATTGTGCCGAGTCCCATATCCGAATGATTAAGACTGGAGAAAGTGGCATGATAG |
| | TTTTCCGGCTTCTTACAAAAAACAACCGATGGACTTGGGTCCAGTCTAATGCACGCCTGCTTTATAAA |
| | AATGGAAGACCAGATTATATCATTGTAACTCAGAGACCACTAACAGATGAGGAAGGAACAGAGCATTT |
| | ACGAAAACGAAATACGAAGTTGCCTTTTATGTTTACCACTGGAGAAGCTGTGTTGTATGAGGCAACCA |
| | ACCCTTTTCCTGCCATAATGGATCCCTTACCATAA |

| | | |
|---|---|---|
| ORF Start: ATG at 1 | | ORF Stop: TAA at 577 |
| SEQ ID NO: 348 | 192 aa | MW at 21913.3 kD |

| | |
|---|---|
| NOV33d, | MNFQGKLKYLHGQKKKGKDGSILPPQLALFAIATPLQPPSILEIRTKNFIFRTKHKLDFTPIGCDAKG |
| CG105355-04 | |
| Protein | RIVLGYTEAELCTRGSGYQFIYAADMLYCAESHIRMIKTGESGMIVFRLLTKNNRWTWVQSNARLLYK |
| Sequence | |
| | NGRPDYIIVTQRPLTDEEGTEHLRKRNTKLPFMFTTGEAVLYEATNPFPAIMDPLP |

Sequence comparison of the above protein sequences yields the following sequence relationships shown in Table 33B.

TABLE 33B
Comparison of NOV33a against NOV33b through NOV33d.

| | NOV33a Residues/ | Identities/Similarities |
|---|---|---|
| Protein Sequence | Match Residues | for the Matched Region |
| NOV33b | 2 . . . 849 | 848/848 (100%) |
| | 1 . . . 848 | 848/848 (100%) |
| NOV33c | 2 . . . 849 | 848/848 (100%) |
| | 1 . . . 848 | 848/848 (100%) |
| NOV33d | 238 . . . 429 | 191/192 (99%) |
| | 1 . . . 192 | 192/192 (99%) |

Further analysis of the NOV33a protein yielded the following properties shown in Table 33C.

TABLE 33C
Protein Sequence Properties NOV33a

| SignalP | | |
|---|---|---|
| analysis: | No Known Signal Sequence Predicted | |
| PSORT II | PSG: | a new signal peptide prediction method |
| analysis: | | N-region: length 0; pos. chg 0; neg. chg 0 |
| | | H-region: length 13; peak value 1.67 |
| | | PSG score: −2.73 |
| | GvH: | von Heijne's method for signal seq. recognition |
| | | GvH score (threshold: −2.1): −10.76 |
| | | possible cleavage site: between 56 and 57 |
| | >>> Seems to have no N-terminal signal peptide | |
| | ALOM: | Klein et al's method for TM region allocation |
| | | Init position for calculation: 1 |
| | | Tentative number of TMS(s) for the threshold 0.5: 1 |
| | | Number of TMS(s) for threshold 0.5: 0 |
| | | PERIPHERAL Likelihood = 2.49 (at 257) |
| | | ALOM score: −0.22 (number of TMSs: 0) |

MITDISC: discrimination of mitochondrial targeting seq

| | | | |
|---|---|---|---|
| R content: | 3 | Hyd Moment | 5.93 |
| Hyd Moment (95): | 4.06 | (75): | |
| D/E content: | 1 | G content: | 0 |
| Score: | −0.41 | S/T content: | 7 |

| Gavel: | prediction of cleavage sites for mitochondrial preseq |
|---|---|
| | R-2 motif at 27 RRK\|PV |
| NUCDISC: discrimination of nuclear localization signals | |
| | pat4: RKRR (5) at 14 |
| | pat4: KRRK (5) at 15 |
| | pat4: RRKP (4) at 16 |
| | pat4: KRHR (3) at 38 |
| | pat7: PSKRHRD (4) at 36 |
| | bipartite: none |
| | content of basic residues: 8.8% |
| | NLS Score: 0.94 |
| KDEL: | ER retention motif in the C-terminus: none |
| ER Membrane Retention Signals: none | |
| SKL: | peroxisomal targeting signal in the C-terminus: none |
| PTS2: | 2nd peroxisomal targeting signal: none |
| VAC: | possible vacuolar targeting motif: none |
| RNA-binding motif: none | |
| Actinin-type actin-binding motif: | |
| | type 1: none |
| | type 2: none |
| NMYR: | N-myristoylation pattern: none |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: | Reinhardt's method for Cytoplasmic/Nuclear |
| | discrimination |
| | Prediction: nuclear |
| | Reliability: 94.1 |
| COIL: | Lupas's algorithm to detect coiled-coil regions |
| | total: 0 residues |

----------------------------------
Final Results (k = 9/23):

```
60.9%: nuclear
26.1%: mitochondrial
  8.7%: peroxisomal
  4.3%: cytoplasmic
```

>> prediction for CG105355-03 is nuc (k = 23)

A search of the NOV33a protein against the Geneseq database, a proprietary database that contains sequences published in patents and patent publication, yielded several homologous proteins shown in Table 33D.

TABLE 33D
Geneseq Results for NOV33a

| | | NOV33a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAW25668 | Human Ah-receptor - Homo sapiens, | 2 . . . 849 | 847/848 (99%) | 0.0 |
| | 848 aa. [US5650283-A, | 1 . . . 848 | 847/848 (99%) | |
| | 22-JUL-1997] | | | |
| AAR80551 | Human Ah receptor protein - Homo | 2 . . . 849 | 847/848 (99%) | 0.0 |
| | sapiens, 848 aa. [US5378822-A, | 1 . . . 848 | 847/848 (99%) | |
| | 03-JAN-1995] | | | |
| AAB73957 | Guinea pig dioxin receptor - Cavia | 2 . . . 849 | 661/852 (77%) | 0.0 |
| | porcellus, 846 aa. [JP2000354494-A, | 1 . . . 846 | 734/852 (85%) | |
| | 26-DEC-2000] | | | |
| AAR80561 | Murine Ah receptor protein - Mus | 4 . . . 805 | 590/814 (72%) | 0.0 |
| | musculus, 805 aa. [US5378822-A, | 2 . . . 805 | 675/814 (82%) | |
| | 03-JAN-1995] | | | |
| ABB08868 | Cricetulus griseus dioxin receptor | 4 . . . 849 | 573/960 (59%) | 0.0 |
| | SEQ ID NO 1 - Cricetulus griseus, | 2 . . . 941 | 663/960 (68%) | |
| | 941 aa. [JP2002045188-A, | | | |
| | 12-FEB-2002] | | | |

In a BLAST search of public sequence datbases, the NOV33a protein was found to have homology to the proteins shown in the BLASTP data in Table 33E.

TABLE 33E
Public BLASTP Results for NOV33a

| | | NOV33a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P35869 | Ah receptor (Aryl hydrocarbon | 2 . . . 849 | 848/848 (100%) | 0.0 |
| | receptor) (AhR) - Homo sapiens | 1 . . . 848 | 848/848 (100%) | |
| | (Human), 848 aa. | | | |
| Q95LD9 | Aryl hydrocarbon receptor - | 2 . . . 849 | 713/854 (83%) | 0.0 |
| | Delphinapterus leucas (Beluga | 1 . . . 845 | 767/854 (89%) | |
| | whale), 845 aa. | | | |
| Q8MKI7 | Aryl hydrocarbon receptor - Phoca | 2 . . . 849 | 679/851 (79%) | 0.0 |
| | sibirica (Baikal seal), 843 aa. | 1 . . . 843 | 740/851 (86%) | |
| O02747 | Ah receptor (Aryl hydrocarbon | 2 . . . 849 | 669/852 (78%) | 0.0 |
| | receptor) (AhR) - Oryctolagus | 1 . . . 847 | 734/852 (85%) | |
| | cuniculus (Rabbit), 847 aa. | | | |
| Q95M15 | Aryl hydrocarbon receptor - Phoca | 2 . . . 849 | 676/851 (79%) | 0.0 |
| | vitulina (Harbor seal), 843 aa. | 1 . . . 843 | 740/851 (86%) | |

PFam analysis predicts that the NOV33a protein contains the domains shown in the Table 33F.

TABLE 33F
Domain Analysis of NOV33a

| | | Identities/ | |
|---|---|---|---|
| | | Similarities | |
| Pfam | NOV33a | for the Matched | Expect |
| Domain | Match Region | Region | Value |
| PAS | 114 . . . 178 | 20/69 (29%) | 1.6e−13 |
| | | 54/69 (78%) | |
| PAC | 349 . . . 390 | 10/43 (23%) | 1.3e−08 |
| | | 37/43 (86%) | |

### Example 34

The NOV34 clone was analyzed, and the nucleotide and encoded polypeptide sequences are shown in Table 34A.

TABLE 34A
NOV34 Sequence Analysis

| | |
|---|---|
| SEQ ID NO: 349 | 2017 bp |

| | |
|---|---|
| NOV34a, | CGTACAACTCCGCCCCATTGACGCAAATGGGCGGTAGGCGTGTACGGTGGGAGGTCTATATAAGCAGAG |
| CG96736-02 | |
| DNA | CTCTCTGGCTAACTAGAGAACCCACTGCTTACTGGCTTATCGAAATTAATACGACTCACTATAGGGAGA |
| Sequence | |
| | CCCAAGCTGGCTAGCGTTTAAACTTAAGCTTGGTACCGAGCTCGGATCCACTAGTCCAGTGTGGTGGAA |
| | TTCCACCATGGTGGCCGATCCTCCTCGAGACTCCAAGGGGCTCGCAGCGGCGGAGCCCACCGCCAACGG |
| | GGGCCTGGCGCTGGCCTCCATCGAGGACCAAGGCGCGGCAGCAGGCGGCTACTGCGGTTCCCGGGACCA |
| | GGTGCGCCGCTGCCTTCGAGCCAACCTGCTTGTGCTGCTGACAGTGGTGGCCGTGGTGGCCGGCGTGGC |
| | GCTGGGACTGGGGGTGTCGGGGGCCGGGGGTGCGCTGGCGTTGGGCCCGGAGCGCTTGAGCGCCTTCGT |
| | CTTCCCGGGCGAGCTGCTGCTGCGTCTGCTGCGGATGATCATCTTGCCGCTGGTGGTGTGCAGCTTGAT |
| | CGGCGGCGCCGCCAGCCTGGACCCCGGCGCGCTCGGCCGTCTGGGCGCCTGGGCGCTGCTCTTTTTCCT |
| | GGTCACCACGCTGCTGGCGTCGGCGCTCGGAGTGGGCTTGGCGCTGGCTCTGCAGCCGGGCGCCGCCTC |
| | CGCCGCCATCAACGCCTCCGTGGGAGCCGCGGGCAGTGCCGAAAATGCCCCCAGCAAGGAGGTGCTCGA |
| | TTCGTTCCTGGATCTTGCGAGAAATATCTTCCCTTCCAACCTGGTGTCAGCAGCCTTTCGCTCATACTC |
| | TACCACCTATGAAGAGAGGAATATCACCGGAACCAGGGTGAAGGTGCCCGTGGGGCAGGAGGTGGAGGG |
| | GATGAACATCCTGGGCTTGGTAGTGTTTGCCATCGTCTTTGGTGTGGCGCTGCGGAAGCTGGGGCCTGA |
| | AGGGGAGCTGCTTATCCGCTTCTTCAACTCCTTCAATGAGGCCACCATGGTTCTGGTCTCCTGGATCAT |
| | GTGGTATGCCCCTGTGGGCATCATGTTCCTGGTGGCTGGCAAGATCGTGGAGATGGAGGATGTGGGTTT |
| | ACTCTTTGCCCGCCTTGGCAAGTACATTCTGTGCTGCCTGCTGGGTCACGCCATCCATGGGCTCCTGGT |
| | ACTGCCCCTCATCTACTTCCTCTTCACCCGCAAAAACCCCTACCGCTTCCTGTGGGGCATCGTGACGCC |
| | GCTGGCCACTGCCTTTGGGACCTCTTCCAGTTCCGCCACGCTGCCGCTGATGATGAAGTGCGTGGAGGA |
| | GAATAATGGCGTGGCCAAGCACATCAGCCGTTTCATCCTGCCCATCGGCGCCACCGTCAACATGGACGG |
| | TGCCGCGCTCTTCCAGTGCGTGGCCGCAGTGTTCATTGCACAGCTCAGCCAGCAGTCCTTGGACTTCGT |
| | AAAGATCATCACCATCCTGGTCACGGCCACAGCGTCCAGCGTGGGGGCAGCGGGCATCCCTGCTGGAGG |
| | TGTCCTCACTCTGGCCATCATCCTCGAAGCAGTCAACCTCCCGGTCGACCATATCTCCTTGATCCTGGC |
| | TGTGGACTGGCTAGTCGACCGGTCCTGTACCGTCCTCAATGTAGAAGGTGACGCTCTGGGGGCAGGACT |
| | CCTCCAAAATTACGTGGACCGTACGGAGTCGAGAAGCACAGAGCCTGAGTTGATACAAGTGAAGAGTGA |
| | GCTGCCCCTGGATCCGCTGCCAGTCCCCACTGAGGAAGGAAACCCCCTCCTCAAACACTATCGGGGGCC |
| | CGCAGGGGATGCCACGGTCGCCTCTGAGAAGGAATCAGTCATGTAAGCGGCCGCTCGAGTCTAGAGGGC |
| | CCGTTTAAACCCGCTGATCAGCCTCGACTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATAAAATGAGGAAATTGCAT |
| | CGCATTGTCTGAGTAG |

| | | |
|---|---|---|
| ORF Start: at 134 | | ORF Stop: TAA at 1838 |
| SEQ ID NO: 350 | 568 aa | MW at 59557.8 kD |

| | |
|---|---|
| NOV34a, | GDPSWLAFKLKLGTELGSTSPVWWNSTMVADPPRDSKGLAAAEPTANGGLALASIEDQGAAAGGYCGSR |
| CG96736-02 | |
| Protein | DQVRRCLRANLLVLLTVVAVVAGVALGLGVSGAGGALALGPERLSAFVFPGELLLRLLRMIILPLVVCS |
| Sequence | |
| | LIGGAASLDPGALGRLGAWALLFFLVTTLLASALGVGLALALQPGAASAAINASVGAAGSAENAPSKEV |
| | LDSFLDLARNIFPSNLVSAAFRSYSTTYEERNITGTRVKVPVGQEVEGMNILGLVVFAIVFGVALRKLG |
| | PEGELLIRFFNSFNEATMVLVSWIMWYAPVGIMFLVAGKIVEMEDVGLLFARLGKYILCCLLGHAIHGL |
| | LVLPLIYFLFTRKNPYRFLWGIVTPLATAFGTSSSSATLPLMMKCVEENNGVAKHISRFILPIGATVNM |
| | DGAALFQCVAAVFIAQLSQQSLDFVKIITILVTATASSVGAAGIPAGGVLTLAIILEAVNLPVDHISLI |
| | LAVDWLVDRSCTVLNVEGDALGAGLLQNYVDRTESRSTEPELIQVKSELPLDPLPVPTEEGNPLLKHYR |
| | GPAGDATVASEKESVM |

| | |
|---|---|
| SEQ ID NO: 351 | 2885 bp |

| | |
|---|---|
| NOV34b, | CGGCACGCCCGGGAGGCTTTCTCTGGCTGGTAACCGCTACTCCCGGACACCAGACCACCGCCTTCCGTA |
| CG96736-01 | |
| DNA | CACAGGGGCCCGCATCCCACCCTCCCGGACCTAAGAGCCTGGGTCCCCTGTTTCCGGAGTCCGCTTCCC |
| Sequence | |
| | GGCCCCCAGATTCTGGCATCCCAGCCCTCAGTGTCCAAGACCCAGGCAGCCCGGGTCCCCGCCTCCCGG |
| | ATCCAGGCGTCCGGGATCTGCGCCACCAGAACCTAGCCTCCTGCAGACCTCCGCCATCTGGGGGCACTC |
| | AACCTCCTGGAGCCAAGGGCCCCACGTCCCACCCAGAGAAACTCTCGTATTCCCAGCTCCTAGGGCCAA |
| | GGAACCCGGGCGCTCCGAACTCCCAGCTTTCGGACATCTGGCACACGGGGCAGAGCAGAGAAGCCTCAG |
| | CGCCCAGCCTGGGGAATTTAAACACTCCAGCTTCCAAGAGCCAAGGAACTTCAGTGCTGTGAACTCACA |
| | ACTCTAAGGAGCCCTCCAAAGTTCCAGTCTCCAGGTGCTGTTACTCAACTCAGTCCTAGGAACGTCGGG |
| | TCCTGGGAAGGAGCCCAAGCGCTCCCAGCCAGCTTCCAGGCGCTAAGAAACCCCGGTGCTTCCCATCAT |
| | GGTGGCCGATCCTCCTCGAGACTCCAAGGGGCTCGCAGCGGCGGAGCCACCGCCAACGGGGGCCTGGCA |
| | GCTGGCCTCCATCGAGGACCAAGGCGCGGCAGCAGGCGGCTACTGCGGTTCCCGGGACCTGGTGCGCCG |
| | CTGCCTTCGAGCCAACCTGCTTGTGCTGCTGACAGTGGTGGCCGTGGTGGCCGGCGTGGCGCTGGGACT |
| | GGGGGTGTCGGGGGCCGGGGGTGCGCTGGCGTTGGGCCCGGGAGCGCTTGAGGCCTTCGTCTTCCCGGG |
| | CGAGCTGCTGCTGCGTCTGCTGCGGATGATCATCTTGCCGCTGGTGGTGTGCAGCTTGATCGGCGGCGC |
| | CGCCAGCCTGGACCCCGGCGCGCTCGGCCGTCTGGGCGCCTGGGCGCTGCTCTTTTTCCTGGTCACCAC |
| | GCTGCTGGCGTCGGCGCTCGGAGTGGGCTTGGCGCTGGCTCTGCAGCCGGGCGCCGCCTCCGCCGCCAT |
| | CAACGCCTCCGTGGGAGCCGCGGGCAGTGCCGAAAATGCCCCCAGCAAGGAGGTGCTCGATTCGTTCCT |
| | GGATCTTGCGAGAAATATCTTCCCTTCCAACCTGGTGTCAGCAGCCTTTCGCTCATACTCTACCACCTA |
| | TGAAGAGAGGAATATCACCGGAACCAGGGTGAAGGTGCCCGTGGGGCAGGAGGTGGAGGGGATGAACAT |
| | CCTGGGCTTGGTAGTGTTTGCCATCGTCTTTGGTGTGGCGCTGCGGAAGCTGGGGCCTGAAGGGGAGCT |
| | GCTTATCCGCTTCTTCAACTCCTTCAATGAGGCCACCATGGTTCTGGTCTCCTGGATCATGTGGTACGC |
| | CCCTGTGGGCATCATGTTCCTGGTGGCTGGCAAGATCGTGGAGATGGAGGATGTGGGTTTACTCTTTGC |
| | CCGCCTTGGCAAGTACATTCTGTGCTGCCTGCTGGGTCACGCCATCCATGGGCTCCTGGTACTGCCCCT |
| | CATCTACTTCCTCTTCACCCGCAAAAACCCCTACCGCTTCCTGTGGGGCATCGTGACGCCGCTGGCCAC |
| | TGCCTTTGGGACCTCTTCCAGTTCCGCCACGCTGCCGCTGATGATGAAGTGCGTGGAGGAGAATAATGG |
| | CGTGGCCAAGCACATCAGCCGTTTCATCCTGCCCATCGGCGCCACCGTCAACATGGACGGTGCCGCGCT |
| | CTTCCAGTGCGTGGCCGCAGTGTTCATTGCACAGCTCAGCCAGCAGTCCTTGGACTTCGTAAAGATCAT |
| | CACCATCCTGGTCACGGCCACAGCGTCCAGCGTGGGGGCAGCGGGCATCCCTGCTGGAGGTGTCCTCAC |
| | TCTGGCCATCATCCTCGAAGCAGTCAACCTCCCGGTCGACCATATCTCCTTGATCCTGGCTGTGGACTG |
| | GCTAGTCGACCGGTCCTGTACCGTCCTCAATGTAGAAGGTGACGCTCTGGGGGCAGGACTCCTCCAAAA |
| | TTATGTGGACCGTACGGAGTCGAGAAGCACAGAGCCTGAGTTGATACAAGTGAAGAGTGAGCTGCCCCT |
| | GGATCCGCTGCCAGTCCCCACTGAGGAAGGAAACCCCCTCCTCAAACACTATCGGGGGCCCGCAGGGGA |
| | TGCCACGGTCGCCTCTGAGAAGGAATCAGTCATGTAAACCCCGGGAGGGACCTTCCCTGCCCTGCTGGG |
| | GGTGCTCTTTGGACACTGGATTATGAGGAATGGATAAATGGATGAGCTAGGGCTCTGGGGGTCTGCCTG |
| | CACACTCTGGGGAGCCAGGGGCCCCAGCACCCTCCAGGACAGGAGATCTGGGATGCCTGGCTGCTGGAG |
| | TACATGTGTTCACAAGGGTTACTCCTCAAAACCCCCAGTTCTCACTCATGTCCCCAACTCAAGGCTAGA |
| | AAACAGCAAGATGGAGAAATAATGTTCTGCTGCGTCCCCACCGTGACCTGCCTGGCCTCCCCTGTCTCA |
| | GGGAGCAGGTCACAGGTCACCATGGGGAATTCTAGCCCCCACTGGGGGGATGTTACAACACCATGCTGG |
| | TTATTTTGGCGGCTGTAGTTGTGGGGGGATGTGTGTGTGCACGTGTGTGTGTGTGTGTGTGTGTGTGTG |
| | TGTGTGTGTTCTGTGACCTCCTGTCCCCATGGTACGTCCCACCCTGTCCCCAGATCCCCTATTCCCTCC |
| | ACAATAACAGAAACACTCCCAGGGACTCTGGGGAGAGGCTGAGGACAAATACCTGCTGTCACTCCAGAG |
| | GACATTTTTTTTAGCAATAAAATTGAGTGTCAACTATTAAAAAAAAAAAAAAAAAA |

| | | |
|---|---|---|
| ORF Start: ATG at 620 | | ORF Stop: TAA at 2243 |
| SEQ ID NO: 352 | 541 aa | MW at 56620.6 kD |

| | |
|---|---|
| NOV34b, | MVADPPRDSKGLAAAEPPPTGAWQLASIEDQGAAAGGYCGSRDLVRRCLRANLLVLLTVVAVVAGVALG |
| CG96736-01 | |
| Protein | LGVSGAGGALALGPGALEAFVFPGELLLRLLRMIILPLVVCSLIGGAASLDPGALGRLGAWALLFFLVT |
| Sequence | |
| | TLLASALGVGLALALQPGAASAAINASVGAAGSAENAPSKEVLDSFLDLARNIFPSNLVSAAFRSYSTT |
| | YEERNITGTRVKVPVGQEVEGMNILGLVVFAIVFGVALRKLGPEGELLIRFFNSFNEATMVLVSWIMWY |
| | APVGIMFLVAGKIVEMEDVGLLFARLGKYILCCLLGHAIHGLLVLPLIYFLFTRKNPYRFLWGIVTPLA |
| | TAFGTSSSSATLPLMMKCVEENNGVAKHISRFILPIGATVNMDGAALFQCVAAVFIAQLSQQSLDFVKI |
| | ITILVTATASSVGAAGIPAGGVLTLAIILEAVNLPVDHISLILAVDWLVDRSCTVLNVEGDALGAGLLQ |
| | NYVDRTESRSTEPELIQVKSELPLDPLPVPTEEGNPLLKHYRGPAGDATVASEKESVM |

| | |
|---|---|
| SEQ ID NO: 353 | 2017 bp |

| | |
|---|---|
| NOV34c, | CGTACAACTCCGCCCCATTGACGCAAATGGGCGGTAGGCGTGTACGGTGGGAGGTCTATATAAGCAGAG |
| 210203253 | |
| DNA | CTCTCTGGCTAACTAGAGAACCCACTGCTTACTGGCTTATCGAAATTAATACGACTCACTATAGGGAGA |
| Sequence | |
| | CCCAAGCTGGCTAGCGTTTAAACTTAAGCTTGGTACCGAGCTCGGATCCACTAGTCCAGTGTGGTGGAA |
| | TTCCACCATGGTGGCCGATCCTCCTCGAGACTCCAAGGGGCTCGCAGCGGCGGAGCCCACCGCCAACGG |
| | GGGCCTGGCGCTGGCCTCCATCGAGGACCAAGGCGCGGCAGCAGGCGGCTACTGCGGTTCCCGGGACCA |
| | GGTGCGCCGCTGCCTTCGAGCCAACCTGCTTGTGCTGCTGACAGTGGTGGCCGTGGTGGCCGGCGTGGC |
| | GCTGGGACTGGGGGTGTCGGGGGCCGGGGGTGCGCTGGCGTTGGGCCCGGAGCGCTTGAGCGCCTTCGT |
| | CTTCCCGGGCGAGCTGCTGCTGCGTCTGCTGCGGATGATCATCTTGCCGCTGGTGGTGTGCAGCTTGAT |
| | CGGCGGCGCCGCCAGCCTGGACCCCGGCGCGCTCGGCCGTCTGGGCGCCTGGGCGCTGCTCTTTTTCCT |
| | GGTCACCACGCTGCTGGCGTCGGCGCTCGGAGTGGGCTTGGCGCTGGCTCTGCAGCCGGGCGCCGCCTC |
| | CGCCGCCATCAACGCCTCCGTGGGAGCCGCGGGCAGTGCCGAAAATGCCCCCAGCAAGGAGGTGCTCGA |
| | TTCGTTCCTGGATCTTGCGAGAAATATCTTCCCTTCCAACCTGGTGTCAGCAGCCTTTCGCTCATACTC |
| | TACCACCTATGAAGAGAGGAATATCACCGGAACCAGGGTGAAGGTGCCCGTGGGGCAGGAGGTGGAGGG |
| | GATGAACATCCTGGGCTTGGTAGTGTTTGCCATCGTCTTTGGTGTGGCGCTGCGGAAGCTGGGGCCTGA |
| | AGGGGAGCTGCTTATCCGCTTCTTCAACTCCTTCAATGAGGCCACCATGGTTCTGGTCTCCTGGATCAT |
| | GTGGTATGCCCCTGTGGGCATCATGTTCCTGGTGGCTGGCAAGATCGTGGAGATGGAGGATGTGGGTTT |
| | ACTCTTTGCCCGCCTTGGCAAGTACATTCTGTGCTGCCTGCTGGGTCACGCCATCCATGGGCTCCTGGT |
| | ACTGCCCCTCATCTACTTCCTCTTCACCCGCAAAAACCCCTACCGCTTCCTGTGGGGCATCGTGACGCC |
| | GCTGGCCACTGCCTTTGGGACCTCTTCCAGTTCCGCCACGCTGCCGCTGATGATGAAGTGCGTGGAGGA |
| | GAATAATGGCGTGGCCAAGCACATCAGCCGTTTCATCCTGCCCATCGGCGCCACCGTCAACATGGACGG |
| | TGCCGCGCTCTTCCAGTGCGTGGCCGCAGTGTTCATTGCACAGCTCAGCCAGCAGTCCTTGGACTTCGT |
| | AAAGATCATCACCATCCTGGTCACGGCCACAGCGTCCAGCGTGGGGGCAGCGGGCATCCCTGCTGGAGG |
| | TGTCCTCACTCTGGCCATCATCCTCGAAGCAGTCAACCTCCCGGTCGACCATATCTCCTTGATCCTGGC |
| | TGTGGACTGGCTAGTCGACCGGTCCTGTACCGTCCTCAATGTAGAAGGTGACGCTCTGGGGGCAGGACT |
| | CCTCCAAAATTACGTGGACCGTACGGAGTCGAGAAGCACAGAGCCTGAGTTGATACAAGTGAAGAGTGA |
| | GCTGCCCCTGGATCCGCTGCCAGTCCCCACTGAGGAAGGAAACCCCCTCCTCAAACACTATCGGGGGCC |
| | CGCAGGGGATGCCACGGTCGCCTCTGAGAAGGAATCAGTCATGTAAGCGGCCGCTCGAGTCTAGAGGGC |
| | CCGTTTAAACCCGCTGATCAGCCTCGACTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATAAAATGAGGAAATTGCAT |
| | CGCATTGTCTGAGTAG |

| | | |
|---|---|---|
| ORF Start: at 134 | | ORF Stop: TAA at 1838 |
| SEQ ID NO: 354 | 568 aa | MW at 59557.8 kD |

| | |
|---|---|
| NOV34c, | GDPSWLAFKLKLGTELGSTSPVWWNSTMVADPPRDSKGLAAAEPTANGGLALASIEDQGAAAGGYCGSR |
| 210203253 | |
| Protein | DQVRRCLRANLLVLLTVVAVVAGVALGLGVSGAGGALALGPERLSAFVFPGELLLRLLRMIILPLVVCS |
| Sequence | |
| | LIGGAASLDPGALGRLGAWALLFFLVTTLLASALGVGLALALQPGAASAAINASVGAAGSAENAPSKEV |
| | LDSFLDLARNIFPSNLVSAAFRSYSTTYEERNITGTRVKVPVGQEVEGMNILGLVVFAIVFGVALRKLG |
| | PEGELLIRFFNSFNEATMVLVSWIMWYAPVGIMFLVAGKIVEMEDVGLLFARLGKYILCCLLGHAIHGL |
| | LVLPLIYFLFTRKNPYRFLWGIVTPLATAFGTSSSSATLPLMMKCVEENNGVAKHISRFILPIGATVNM |
| | DGAALFQCVAAVFIAQLSQQSLDFVKIITILVTATASSVGAAGIPAGGVLTLAIILEAVNLPVDHISLI |
| | LAVDWLVDRSCTVLNVEGDALGAGLLQNYVDRTESRSTEPELIQVKSELPLDPLPVPTEEGNPLLKHYR |
| | GPAGDATVASEKESVM |

| | |
|---|---|
| SEQ ID NO: 355 | 2022 bp |

| | |
|---|---|
| NOV34d, | ATGTGTAACAACTCCGCCCCATTGACGCAAATGGGCGGTAGGCGTGTACGGTGGGAGGTCTATATAAGC |
| 210203261 | |
| DNA | AGAGCTCTCTGGCTAACTAGAGAACCCACTGCTTACTGGCTTATCGAAATTAATACGACTCACTATAGG |
| Sequence | |
| | GAGACCCAAGCTGGCTAGCGTTTAAACTTAAGCTTGGTACCGAGCTCGGATCCACTAGTCCAGTGTGGT |
| | GGAATTCCACCATGGTGGCCGATCCTCCTCGAGACTCCAAGGGGCTCGCAGCGGCGGAGCCCACCGCCA |
| | ACGGGGGCCTGGCGCTGGCCTCCATCGAGGACCAAGGCGCGGCAGCAGGCGGCTACTGCGGTTCCCGGG |
| | ACCAGGTGCGCCGCTGCCTTCGAGCCAACCTGCTTGTGCTGCTGACAGTGGTGGCCGTGGTGGCCGGCG |
| | TGGCGCTGGGACTGGGGGTGTCGGGGGCCGGGGGTGCGCTGGCGTTGGGCCCGGAGCGCTTGAGCGCCT |
| | TCGTCTTCCCGGGCGAGCTGCTGCTGCGTCTGCTGCGGATGATCATCTTGCCGCTGGTGGTGTGCAGCT |
| | TGATCGGCGGCGCCGCCAGCCTGGACCCCGGCGCGCTCGGCCGTCTGGGCGCCTGGGCGCTGCTCTTTT |
| | TCCTGGTCACCACGCTGCTGGCGTCGGCGCTCGGAGTGGGCTTGGCGCTGGCTCTGCAGCCGGGCGCCG |
| | CCTCCGCCGCCATCAACGCCTCCGTGGGAGCCGCGGGCAGTGCCGAAAATGCCCCCAGCAAGGAGGTGC |
| | TCGATTCGTTCCTGGATCTTGCGAGAAATATCTTCCCTTCCAACCTGGTGTCAGCAGCCTTTCGCTCAT |
| | ACTCTACCACCTATGAAGAGAGGAATATCACCGGAACCAGGGTGAAGGTGCCCGTGGGGCAGGAGGTGG |
| | AGGGGATGAACATCCTGGGCTTGGTAGTGTTTGCCATCGTCTTTGGTGTGGCGCTGCGGAAGCTGGGGC |
| | CTGAAGGGGAGCTGCTTATCCGCTTCTTCAACTCCTTCAATGAGGCCACCATGGTTCTGGTCTCCTGGA |
| | TCATGTGGTACGCCCCTGTGGGCATCATGTTCCTGGTGGCTGGCAAGATCGTGGAGATGGAGGATGTGG |
| | GTTTACTCTTTGCCCGCCTTGGCAAGTACATTCTGTGCTGCCTGCTGGGTCACGCCATCCATGGGCTCC |
| | TGGTACTGCCCCTCATCTACTTCCTCTTCACCCGCAAAAACCCCTACCGCTTCCTGTGGGGCATCGTGA |
| | CGCCGCTGGCCACTGCCTTTGGGACCTCTTCCAGTTCCGCCACGCTGCCGCTGATGATGAAGTGCGTGG |
| | AGGAGAATAATGGCGTGGCCAAGCACATCAGCCGTTTCATCCTGCCCATCGGCGCCACCGTCAACATGG |
| | ACGGTGCCGCGCTCTTCCAGTGCGTGGCCGCAGTGTTCATTGCACAGCTCAGCCAGCAGTCCTTGGACT |
| | TCGTAAAGATCATCACCATCCTGGTCACGGCCACAGCGTCCAGCGTGGGGGCAGCGGGCATCCCTGCTG |
| | GAGGTGTCCTCACTCTGGCCATCATCCTCGAAGCAGTCAACCTCCCGGTCGACCATATCTCCTTGATCC |
| | TGGCTGTGGACTGGCTAGTCGACCGGTCCTGTACCGTCCTCAATGTAGAAGGTGACGCTCTGGGGGCAG |
| | GACTCCTCCAAAATTACGTGGACCGTACGGAGTCGAGAAGCACAGAGCCTGAGTTGATACAAGTGAAGA |
| | GTGAGCTGCCCCTGGATCCGCTGCCACTCCCCACTGAGGAAGGAAACCCCCTCCTCAAACACTATCGGG |
| | GGCCCGCAGGGGATGCCACGGTCGCCTCTGAGAAGGAATCAGTCATGTAAGCGGCCGCTCGAGTCTAGA |
| | GGGCCCGTTTAAACCCGCTGATCAGCCTCGACTGTGCCTTCTAGTTGCCAGCCATCTGTTGTTTGCCCC |
| | TCCCCCGTGCCTTCCTTGACCCTGGAAGGTGCCACTCCCACTGTCCTTTCCTAATAAAATGAGGAAATT |
| | GCATCGCATTGTCTGAGTAGG |

| | | |
|---|---|---|
| ORF Start: at 138 | | ORF Stop: TAA at 1842 |
| SEQ ID NO: 356 | 568 aa | MW at 59571.8 kD |

| | |
|---|---|
| NOV34d, | GDPSWLAFKLKLGTELGSTSPVWWNSTMVADPPRDSKGLAAAEPTANGGLALASIEDQGAAAGGYCGSR |
| 210203261 | |
| Protein | DQVRRCLRANLLVLLTVVAVVAGVALGLGVSGAGGALALGPERLSAFVFPGELLLRLLRMIILPLVVCS |
| Sequence | |
| | LIGGAASLDPGALGRLGAWALLFFLVTTLLASALGVGLALALQPGAASAAINASVGAAGSAENAPSKEV |
| | LDSFLDLARNIFPSNLVSAAFRSYSTTYEERNITGTRVKVPVGQEVEGMNILGLVVFAIVFGVALRKLG |
| | PEGELLIRFFNSFNEATMVLVSWIMWYAPVGIMFLVAGKIVEMEDVGLLFARLGKYILCCLLGHAIHGL |
| | LVLPLIYFLFTRKNPYRFLWGIVTPLATAFGTSSSSATLPLMMKCVEENNGVAKHISRFILPIGATVNM |
| | DGAALFQCVAAVFIAQLSQQSLDFVKIITILVTATASSVGAAGIPAGGVLTLAIILEAVNLPVDHISLI |
| | LAVDWLVDRSCTVLNVEGDALGAGLLQNYVDRTESRSTEPELIQVKSELPLDPLPLPTEEGNPLLKHYR |
| | GPAGDATVASEKESVM |

Sequence comparison of the above protein sequences yields the following sequence relationships shown in Table 34B.

TABLE 34B
Comparison of NOV34a against NOV34b through NOV34d.

| | | Identities/ |
|---|---|---|
| | NOV34a Residues/ | Similarities for the |
| Protein Sequence | Match Residues | Matched Region |
| NOV34b | 28 . . . 568 | 531/541 (98%) |
| | 1 . . . 541 | 531/541 (98%) |
| NOV34c | 1 . . . 568 | 568/568 (100%) |
| | 1 . . . 568 | 568/568 (100%) |
| NOV34d | 1 . . . 568 | 567/568 (99%) |
| | 1 . . . 568 | 568/568 (99%) |

Further analysis of the NOV34a protein yielded the following properties shown in Table 34C.

TABLE 34C
Protein Sequence Properties NOV34a

| | |
|---|---|
| SignalP analysis: | No Known Signal Sequence Predicted |
| PSORT II analysis: | PSG: a new signal peptide prediction method |
| | N-region: length 11; pos.chg 2; neg.chg 1 |
| | H-region: length 3; peak value 1.25 |
| | PSG score: −3.15 |
| | GvH: von Heijne's method for signal seq. recognition |
| | GvH score (threshold: −2.1): −8.49 |
| | possible cleavage site: between 61 and 62 |
| | >>> Seems to have no N-terminal signal peptide |
| | ALOM: Klein et al's method for TM region allocation |
| | Init position for calculation: 1 |
| | Tentative number of TMS(s) for the threshold 0.5: 8 |
| | INTEGRAL Likelihood = −11.25 Transmembrane 80-96 |
| | INTEGRAL Likelihood = −6.53 Transmembrane 130-146 |
| | INTEGRAL Likelihood = −6.74 Transmembrane 158-174 |
| | INTEGRAL Likelihood = −9.08 Transmembrane 256-272 |
| | INTEGRAL Likelihood = −2.55 Transmembrane 295-311 |
| | INTEGRAL Likelihood = −3.93 Transmembrane 332-348 |
| | INTEGRAL Likelihood = −1.33 Transmembrane 416-432 |
| | INTEGRAL Likelihood = −3.13 Transmembrane 453-469 |
| | PERIPHERAL Likelihood = 1.11 (at 435) |
| | ALOM score: −11.25 (number of TMSs: 8) |
| | MTOP: Prediction of membrane topology (Hartmann et al.) |
| | Center position for calculation: 87 |
| | Charge difference: −3.0 C(0.0) − N(3.0) |
| | N >= C: N-terminal side will be inside |
| | >>> membrane topology: type 3a |
| | MITDISC: discrimination of mitochondrial targeting seq |

| | | | |
|---|---|---|---|
| R content: | 0 | Hyd Moment(75): | 4.14 |
| Hyd Moment(95): | 5.72 | G content: | 2 |
| D/E content: | 2 | S/T content: | 2 |
| Score: | −7.71 | | |

Gavel: prediction of cleavage sites for mitochondrial preseq
cleavage site motif not found
NUCDISC: discrimination of nuclear localization signals
pat4: none
pat7: none
bipartite: none
content of basic residues: 6.7%
NLS Score: −0.47
KDEL: ER retention motif in the C-terminus: none
ER Membrane Retention Signals:
KKXX-like motif in the C-terminus: KESV
SKL: peroxisomal targeting signal in the C-terminus: none
PTS2: 2nd peroxisomal targeting signal: none
VAC: possible vacuolar targeting motif: found
ILPI at 405
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
66.7%: endoplasmic reticulum
22.2%: mitochondrial
11.1%: vesicles of secretory system
>> prediction for CG96736-02 is end (k = 9)

A search of the NOV34a protein against the Geneseq database, a proprietary database that contains sequences published in patents and patent publication, yielded several homologous proteins shown in Table 34D.

TABLE 34D
Geneseq Results for NOV34a

| | | NOV34a | | |
|---|---|---|---|---|
| | | Residues/ | Identities/ | |
| Geneseq | Protein/Organism/Length [Patent | Match | Similarities for the | Expect |
| Identifier | #, Date] | Residues | Matched Region | Value |
| ABG61858 | Prostate cancer-associated protein | 28 . . . 568 | 541/541 (100%) | 0.0 |
| | #59 - Mammalia, 541 aa. | 1 . . . 541 | 541/541 (100%) | |
| | [WO200230268-A2, 18-APR-2002] | | | |
| AAR95044 | Apoptosis participating protein - | 28 . . . 540 | 509/513 (99%) | 0.0 |
| | Homo sapiens, 514 aa. | 1 . . . 513 | 509/513 (99%) | |
| | [JP08089257-A, 09-APR-1996] | | | |
| AAU80097 | Human solute carrier family 1, | 59 . . . 568 | 315/521 (60%) | e−162 |
| | SLC1A4 - Homo sapiens, 532 aa. | 26 . . . 532 | 382/521 (72%) | |
| | [WO200244198-A2, 06-JUN-2002] | | | |
| AAY78144 | Human neutral amino acid | 61 . . . 568 | 311/516 (60%) | e−161 |
| | transporter ASCT1 - Homo sapiens, | 24 . . . 532 | 378/516 (72%) | |
| | 532 aa. [US6020479-A, | | | |
| | 01-FEB-2000] | | | |
| AAY99961 | Human amino acid transporter | 61 . . . 568 | 311/516 (60%) | e−161 |
| | ASCT1 protein - Homo sapiens, 532 | 24 . . . 532 | 378/516 (72%) | |
| | aa. [US6074828-A, 13-JUN-2000] | | | |

In a BLAST search of public sequence datbases, the NOV34a protein was found to have homology to the proteins shown in the BLASTP data in Table 34E.

TABLE 34E
Public BLASTP Results for NOV34a

| | | NOV34a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q15758 | Neutral amino acid transporter B(0) | 28 . . . 568 | 541/541 (100%) | 0.0 |
| | (ATB(0)) (Sodium-dependent neutral | 1 . . . 541 | 541/541 (100%) | |
| | amino acid transporter type 2) | | | |
| | (RD114/simian type D retrovirus | | | |
| | receptor) (Baboon M7 virus receptor) - | | | |
| | Homo sapiens (Human), 541 aa. | | | |
| AAD09814 | Neutral amino acid transporter - | 28 . . . 568 | 540/541 (99%) | 0.0 |
| | Homo sapiens (Human), 541 aa. | 1 . . . 541 | 540/541 (99%) | |
| O19105 | Neutral amino acid transporter B(0) | 28 . . . 568 | 464/542 (85%) | 0.0 |
| | (ATB(0)) (Sodium-dependent neutral | 1 . . . 541 | 490/542 (89%) | |
| | amino acid transporter type 2) - | | | |
| | Oryctolagus cuniculus (Rabbit), 541 | | | |
| | aa. | | | |
| Q95JC7 | Neutral amino acid transporter B(0) | 28 . . . 568 | 469/542 (86%) | 0.0 |
| | (ATB(0)) (Sodium-dependent neutral | 1 . . . 539 | 490/542 (89%) | |
| | amino acid transporter type 2) - Bos | | | |
| | taurus (Bovine), 539 aa. | | | |
| Q8K3F0 | Na+-dependent amino acid | 28 . . . 568 | 451/553 (81%) | 0.0 |
| | transporter ASCT2 - Rattus | 1 . . . 551 | 478/553 (85%) | |
| | norvegicus (Rat), 551 aa. | | | |

PFam analysis predicts that the NOV34a protein contains the domains shown in the Table 34F.

TABLE 34F
Domain Analysis of NOV34a

| | | Identities/ | |
|---|---|---|---|
| | | Similarities | |
| Pfam | NOV34a Match | for the Matched | Expect |
| Domain | Region | Region | Value |
| SDF | 81 . . . 512 | 194/465 (42%) | 2e−178 |
| | | 371/465 (80%) | |

### Example 35

The NOV35 clone was analyzed, and the nucleotide and encoded polypeptide sequences are shown in Table 35A.

TABLE 35A
NOV35 Sequence Analysis

| | |
|---|---|
| SEQ ID NO: 357 | 1612 bp |

| | |
|---|---|
| NOV35a, | ACATCATCACCACCATCACCCTGGATCACTTCCTTTGAATGCAGAAGCTTGCTGGCCAAAAGATGTGG |
| CG97025-04 | |
| DNA | AATTGTTGCCCTTGAGATCTATTTTCCTTCTCAATATGTTGATCAAGCAGAGTTGGAAAAATATGATGG |
| Sequence | |
| | TGTAGATGCTGGAAAGTATACCATTGGCTTGGGCCAGGCCAAGATGGGCTTCTGCACAGATAGAGAAGA |
| | TATTAACTCTCTTTGCATGACTGTGGTTCAGAATCTTATGGAGAGAAATAACCTTTCCTATGATTGCAT |
| | TGGGCGGCTGGAAGTTGGAACAGAGACAATCATCGACAAATCAAAGTCTGTGAAGACTAATTTGATGCA |
| | GCTGTTTGAAGAGTCTGGGAATACAGATATAGAAGGAATCGACACAACTAATGCATGCTATGGAGGCAC |
| | AGCTGCTGTCTTCAATGCTGTTAACTGGATTGAGTCCAGCTCTTGGGATGGACGGTATGCCCTGGTAGT |
| | TGCAGGAGATATTGCTGTATATGCCACAGGAAATGCTAGACCTACAGGTGGAGTTGGAGCAGTAGCTCT |
| | GCTAATTGGGCCAAATGCTCCTTTAATTTTTGAACGAGGGCTTCGTGGGACACATATGCAACATGCCTA |
| | TGATTTTTACAAGCCTGATATGCTATCTGAATATCCTATAGTAGATGGAAAACTCTCCATACAGTGCTA |
| | CCTCAGTGCATTAGACCGCTGCTACTCTGTCTACTGCAAAAAGATCCATGCCCAGTGGCAGAAAGAGGG |
| | AAATGATAAAGATTTTACCTTGAATGATTTTGGCTTCATGATCTTTCACTCACCATATTGTAAACTGGT |
| | TCAGAAATCTCTAGCTCGGATGTTGCTGAATGACTTCCTTAATGACCAGAATAGAGATAAAAATAGTAT |
| | CTATAGTGGCCTGGAAGCCTTTGGGGATGTTAAATTAGAAGACACCTACTTTGATAGAGATGTGGAGAA |
| | GGCATTTATGAAGGCTAGCTCTGAACTCTTCAGTCAGAAAACAAAGGCATCTTTACTTGTATCAAATCA |
| | AAATGGAAATATGTACACATCTTCAGTATATGGTTCCCTTGCATCTGTTCTAGCACAGTACTCACCTCA |
| | GCAATTAGCAGGGAAGAGAATTGGAGTGTTTTCTTATGGTTCTGGTTTGGCTGCCACTCTGTACTCTCT |
| | TAAAGTCACACAAGATGCTACACCGGGGTCTGCTCTTGATAAAATAACAGCAAGTTTATGTGATCTTAA |
| | ATCAAGGCTTGATTCAAGAACTGGTGTGGCACCAGATGTCTTCGCTGAAAACATGAAGCTCAGAGAGGA |
| | CACCCATCATTTGGTCAACTATATTCCCCAGGGTTCAATAGATTCACTCTTTGAAGGAACGTGGTACTT |
| | AGTTAGGGTGGATGAAAAGCACAGAAGAACTTACGCTCGGCGTCCCACTCCAAATGATGACACTTTGGA |
| | TGAAGGAGTAGGACTTGTGCATTCAAACATAGCAACTGAGCATATTCCAAGCCCTGCCAAGAAAGTACC |
| | AAGACTCCCTGCCACAGCAGCAGAACCTGAAGCAGCTGTCATTAGTAATGGGGAACATTAAGCGGCCGC |
| | ACTCGAGCACCACCACCACCACCAC |

| | | |
|---|---|---|
| ORF Start: at 2 | | ORF Stop: TAA at 1577 |
| SEQ ID NO: 358 | 525 aa | MW at 57984.6kD |

| | |
|---|---|
| NOV35a, | HHHHHHPGSLPLNAEACWPKDVGIVALEIYFPSQYVDQAELEKYDGVDAGKYTIGLGQAKMGFCTDRED |
| CG97025-04 | |
| Protein | INSLCMTVVQNLMERNNLSYDCIGRLEVGTETIIDKSKSVKTNLMQLFEESGNTDIEGIDTTNACYGGT |
| Sequence | |
| | AAVFNAVNWIESSSWDGRYALVVAGDIAVYATGNARPTGGVGAVALLIGPNAPLIFERGLRGTHMQHAY |
| | DFYKPDMLSEYPIVDGKLSIQCYLSALDRCYSVYCKKIHAQWQKEGNDKDFTLNDFGFMIFHSPYCKLV |
| | QKSLARMLLNDFLNDQNRDKNSIYSGLEAFGDVKLEDTYFDRDVEKAFMKASSELFSQKTKASLLVSNQ |
| | NGNMYTSSVYGSLASVLAQYSPQQLAGKRIGVFSYGSGLAATLYSLKVTQDATPGSALDKITASLCDLK |
| | SRLDSRTGVAPDVFAENMKLREDTHHLVNYIPQGSIDSLFEGTWYLVRVDEKHRRTYARRPTPNDDTLD |
| | EGVGLVHSNIATEHIPSPAKKVPRLPATAAEPEAAVISNGEH |

| | |
|---|---|
| SEQ ID NO: 359 | 1650 bp |

| | |
|---|---|
| NOV35b, | CCTTCACACAGCTCTTTCACCATGCCTGGATCACTTCCTTTGAATGCAGAAGCTTGCTGGCCAAAAGAT |
| CG97025-01 | |
| DNA | GTTGGGATTGTTGCCCTTGAGATCTATTTTCCTTCTCAATATGTTGATCAAGCAGAGTTGGAAAAATAT |
| Sequence | |
| | GATGGTGTAGATGCTGGGAAGTATACCATTGGCTTGGGCCAGGCCAAGATGGGCTTCTGCACAGATAGA |
| | GAAGATATTAACTCTCTTTGCATGACTGTGGTTCAGAATCTTATGGAGAGAAATAACCTTTCCTATGAT |
| | TGCATTGGGCGGCTGGAAGTTGGAACAGAGACAATCATCGACAAATCAAAGTCTGTGAAGACTAATTTG |
| | ATGCAGCTGTTTGAAGAGTCTGGGAATACAGATATAGAAGGAATCGACACAACTAATGCATGCTATGGA |
| | GGCACAGCTGCTGTCTTCAATGCTGTTAACTGGATTGAGTCCAGCTCTTGGGATGGACGGTATGCCCTG |
| | GTAGTTGCAGGAGATATTGCTGTATATGCCACAGGAAATGCTAGACCTACAGGTGGAGTTGGAGCAGTA |
| | GCTCTGCTAATTGGGCCAAATGCTCCTTTAATTTTTGAACGAGGGCTTCGTGGGACACATATGCAACAT |
| | GCCTATGATTTTTACAAGCCTGATATGCTATCTGAATATCCTATAGTAGATGGGAAACTCTCCATACAG |
| | TGCTACCTCAGTGCATTAGACCGCTGCTATTCTGTCTACTGCAAAAAGATCCATGCCCAGTGGCAGAAA |
| | GAGGGAAATGATAAAGATTTTACCTTGAATGATTTTGGCTTCATGATCTTTCACTCACCATATTGTAAA |
| | CTGGTTCAGAAATCTCTAGCTCGGATGTTGCTGAATGACTTCCTTAATGACCAGAATAGAGATAAAAAT |
| | AGTATCTATAGTGGCCTGGAAGCCTTTGGGGATGTTAAATTAGAAGACACCTACTTTGATAGAGATGTG |
| | GAGAAGGCATTTATGAAGGCTAGCTCTGAACTCTTCAGTCAGAAAACAAAGGCATCTTTACTTGTATCA |
| | AATCAAAATGGAAATATGTACACATCTTCAGTATATGGTTCCCTTGCATCTGTTCTAGCACAGTACTCA |
| | CCTCAGCAATTAGCAGGGAAGAGAATTGGAGTGTTTTCTTATGGTTCTGGTTTGGCTGCCACTCTGTAC |
| | TCTCTTAAAGTCACACAAGATCCTACACCGGGGTCTGCTCTTGATAAAATAACAGCAAGTTTATGTGAT |
| | CTTAAATCAAGGCTTGATTCAAGAACTGGTGTCGCACCAGATGTCTTCGCTGAAAACATGAAGCTCAGA |
| | GAGGACACCCATCATTTGGTCAACTATATTCCCCAGGGTTCAATAGATTCACTCTTTGAAGGAACGTGG |
| | TACTTAGTTAGGGTGGATGAAAAGCACAGAAGAACTTACGCTCGGCGTCCCACTCCAAATGATGACACT |
| | TTGGATGAAGGAGTAGGACTTGTGCATTCAAACATAGCAACTGAGCATATTCCAAGCCCTGCCAAGAAA |
| | GTACCAAGACTCCCTGCCACAGCAGCAGAACCTGAAGCAGCTGTCATTAGTAATGGGGAACATTAAGAT |
| | ACTCTGTGAGGTGCAAGACTTCAGGGTGGGGTGGGCATGGGGTGGGGGTATGGGAACAGTTGG |

| | | |
|---|---|---|
| ORF Start: ATG at 22 | | ORF Stop: TAA at 1582 |
| SEQ ID NO: 360 | 1520 aa | MW at 57293.0kD |

| | |
|---|---|
| NOV35b, | MPGSLPLNAEACWPKDVGIVALEIYFPSQYVDQAELEKYDGVDAGKYTIGLGQAKMGFCTDREDINSLC |
| CG97025-01 | |
| Protein | MTVVQNLMERNNLSYDCIGRLEVGTETIIDKSKSVKTNLMQLFEESGNTDIEGIDTTNACYGGTAAVFN |
| Sequence | |
| | AVNWIESSSWDGRYALVVAGDIAVYATGNARPTGGVGAVALLIGPNAPLIFERGLRGTHMQHAYDFYKP |
| | DMLSEYPIVDGKLSIQCYLSALDRCYSVYCKKIHAQWQKEGNDKDFTLNDFGFMIFHSPYCKLVQKSLA |
| | RMLLNDFLNDQNRDKNSIYSGLEAFGDVKLEDTYFDRDVEKAFMKASSELFSQKTKASLLVSNQNGNMY |
| | TSSVYGSLASVLAQYSPQQLAGKRIGVFSYGSGLAATLYSLKVTQDATPGSALDKITASLCDLKSRLDS |
| | RTGVAPDVFAENMKLREDTHHLVNYIPQGSIDSLFEGTWYLVRVDEKHRRTYARRPTPNDDTLDEGVGL |
| | VHSNIATEHIPSPAKKVPRLPATAAEPEAAVISNGEH |

| | |
|---|---|
| SEQ ID NO: 361 | 1593 bp |

| | |
|---|---|
| NOV35c, | CCTGGATCACTTCCTTTGAATGCAGAAGCTTGCTGGCCAAAAGATGTGGGAATTGTTGCCCTTGAGATC |
| 254869578 | |
| DNA | TATTTTCCTTCTCAATATGTTGATCAAGCAGAGTTGGAAAAATATGATGGTGTAGATGCTGGAAAGTAT |
| Sequence | |
| | ACCATTGGCTTGGGCCAGGCCAAGATGGGCTTCTGCACAGATAGAGAAGATATTAACTCTCTTTGCATG |
| | ACTGTGGTTCAGAATCTTATGGAGAGAAATAACCTTTCCTATGATTGCATTGGGCGGCTGGAAGTTGGA |
| | ACAGAGACAATCATCGACAAATCAAAGTCTCTGAAGACTAATTTGATGCAGCTGTTTGAAGAGTCTGGG |
| | AATACAGATATAGAAGGAATCGACACAACTAATGCATGCTATGGAGGCACAGCTGCTGTCTTCAATGCT |
| | GTTAACTGGATTGAGTCCAGCTCTTGGGATGGACGGTATGCCCTGGTAGTTGCAGGAGATATTGCTGTA |
| | TATGCCACAGGAAATGCTAGACCTACAGGTGGAGTTGGAGCAGTAGCTCTGCTAATTGGGCCAAATGCT |
| | CCTTTAATTTTTGAACGAGGGCTTCGTGGGACACATATGCAACATGCCTATGATTTTTACAAGCCTGAT |
| | ATGCTATCTGAATATCCTATAGTAGATGGAAAACTCTCCATACAGTGCTACCTCAGTGCATTAGACCGC |
| | TGCTACTCTGTCTACTGCAAAAAGATCCATGCCCAGTGGCAGAAAGAGGGAAATGATAAAGATTTTACC |
| | TTGAATGATTTTGGCTTCATGATCTTTCACTCACCATATTGTAAACTGGTTCAGAAATCTCTAGCTCGG |
| | ATGTTGCTGAATGACTTCCTTAATGACCAGAATAGAGATAAAAATAGTATCTATAGTGGCCTGGAAGCC |
| | TTTGGGGATGTTAAATTAGAAGACACCTACTTTGATAGAGATGTGGAGAAGGCATTTATGAAGGCTAGC |
| | TCTGAACTCTTCAGTCAGAAAACAAAGGCATCTTTACTTGTATCAAATCAAAATGGAAATATGTACACA |
| | TCTTCAGTATATGGTTCCCTTGCATCTGTTCTAGCACAGTACTCACCTCAGCAATTAGCAGGGAAGAGA |
| | ATTGGAGTGTTTTCTTATGGTTCTGGTTTGGCTGCCACTCTGTACTCTCTTAAAGTCACACAAGATGCT |
| | ACACCGGGGTCTGCTCTTGATAAAATAACAGCAAGTTTATGTGATCTTAAATCAAGGCTTGATTCAAGA |
| | ACTGGTGTGGCACCAGATGTCTTCGCTGAAAACATGAAGCTCAGAGAGGACACCCATCATTTGGTCAAC |
| | TATATTCCCCAGGGTTCAATAGATTCACTCTTTGAAGGAACGTGGTACTTAGTTAGGGTGGATGAAAAG |
| | CACAGAAGAACTTACGCTCGGCGTCCCACTCCAAATGATGACACTTTGGATGAAGGAGTAGGACTTGTG |
| | CATTCAAACATAGCAACTGAGCATATTCCAAGCCCTGCCAAGAAAGTACCAACACTCCCTGCCACAGCA |
| | GCAGAACCTGAAGCAGCTGTCATTAGTAATGGGGAACATTAAGCGGCCGCACTCGAGCACCACCACCAC |
| | CACCAC |

| | | |
|---|---|---|
| ORF Start: at 1 | | ORF Stop: TAA at 1558 |
| SEQ ID NO: 362 | 519 aa | MW at 57161.8kD |

| | |
|---|---|
| NOV35c, | PGSLPLNAEACWPKDVGIVALEIYFPSQYVDQAELEKYDGVDAGKYTIGLGQAKMGFCTDREDINSLCM |
| 254869578 | |
| Protein | TVVQNLMERNNLSYDCIGRLEVGTETIIDKSKSVKTNLMQLFEESGNTDIEGIDTTNACYGGTAAVFNA |
| Sequence | |
| | VNWIESSSWDGRYALVVAGDIAVYATGNARPTGGVGAVALLIGPNAPLIFERGLRGTHMQHAYDFYKPD |
| | MLSEYPIVDGKLSIQCYLSALDRCYSVYCKKIHAQWQKEGNDKDFTLNDFGFMIFHSPYCKLVQKSLAR |
| | MLLNDFLNDQNRDKNSIYSGLEAFGDVKLEDTYFDRDVEKAFMKASSELFSQKTKASLLVSNQNGNMYT |
| | SSVYGSLASVLAQYSPQQLAGKRIGVFSYGSGLAATLYSLKVTQDATPGSALDKITASLCDLKSRLDSR |
| | TGVAPDVFAENMKLREDTHHLVNYIPQGSIDSLFEGTWYLVRVDEKHRRTYARRPTPNDDTLDEGVGLV |
| | HSNIATEHIPSPAKKVPRLPATAAEPEAAVISNGEH |

| | |
|---|---|
| SEQ ID NO: 363 | 1601 bp |

| | |
|---|---|
| NOV35d, | CACCGGTCTCACATGCCTGGATCACTTCCTTTGAATGCAGAAGCTTGCTGGCCAAAAGATGTGGGAATT |
| 253174237 | |
| DNA | GTTGCCCTTGAGATCTATTTTCCTTCTCAATATGTTGATCAAGCAGAGTTGGAAAAATATGATGGTGTA |
| Sequence | |
| | GATGCTGGAAAGTATACCATTGGCTTGGGCCAGGCCAAGATGGGCTTCTGCACAGATAGAGAAGATATT |
| | AACTCTCTTTGCATGACTGTGGTTCAGAATCTTATGGAGAGAAATAACCTTTCCTATGATTGCATTGGG |
| | CGGCTGGAAGTTGGAACAGAGACAATCATCGACAAATCAAAGTCTGTGAAGACTAATTTGATGCAGCTG |
| | TTTGAAGAGTCTGGGAATACAGATATAGAAGGAATCGACACAACTAATGCATGCTATGGAGGCACAGCT |
| | CCTGTCTTCAATGCTGTTAACTGGATTGAGTCCAGCTCTTCGGATGGACGGTATGCCCTGGTAGTTGCA |
| | GGAGATATTGCTGTATATGCCACAGGAAATGCTAGACCTACAGGTGGAGTTGGAGCAGTAGCTCTGCTA |
| | ATTGGGCCAAATGCTCCTTTAATTTTTGAACGAGGGCTTCGTGGGACACATATGCAACATGCCTATGAT |
| | TTTTACAAGCCTGATATGCTATCTGAATATCCTATAGTAGATGGAAAACTCTCCATACAGTGCTACCTC |
| | AGTGCATTAGACCGCTGCTACTCTGTCTACTGCAAAAAGATCCATGCCCAGTGGCAGAAAGAGGGAAAT |
| | GATAAACATTTTACCTTGAATGATTTTGGCTTCATGATCTTTCACTCACCATATTGTAAACTCGTTCAG |
| | AAATCTCTAGCTCGGATGTTGCTGAATGACTTCCTTAATGACCAGAATAGAGATAAAAATAGTATCTAT |
| | AGTGGCCTGGAAGCCTTTGGGGATGTTAAATTAGAAGACACCTACTTTGATAGAGATGTGGAGAAGGCA |
| | TTTATGAAGGCTAGCTCTGAACTCTTCAGTCAGAAAACAAAGGCATCTTTACTTGTATCAAATCAAAAT |
| | GGAAATATGTACACATCTTCAGTATATGGTTCCCTTGCATCTGTTCTAGCACAGTACTCACCTCAGCAA |
| | TTAGCAGGGAAGAGAATTGGAGTGTTTTCTTATGGTTCTGGTTTGGCTGCCACTCTGTACTCTCTTAAA |
| | GTCACACAAGATGCTACACCGGGGTCTGCTCTTGATAAAATAACAGCAAGTTTATGTGATCTTAAATCA |
| | AGGCTTGATTCAAGAACTGGTGTGGCACCAGATGTCTTCGCTGAAAACATGAAGCTCAGAGAGGACACC |
| | CATCATTTGGTCAACTATATTCCCCAGGGTTCAATAGATTCACTCTTTGAAGGAACGTGGTACTTAGTT |
| | AGGGTGGATGAAAAGCACAGAAGAACTTACGCTCGGCGTCCCACTCCAAATGATGACACTTTGGATGAA |
| | GGAGTAGGACTTGTGCATTCAAACATAGCAACTGAGCATATTCCAAGCCCTGCCAAGAAAGTACCAAGA |
| | CTCCCTGCCACAGCAGCAGAACCTGAAGCAGCTGTCATTAGTAATGGGGAACATCATCACCACCATCAC |
| | TAAGCGGCCGGAAG |

| | | |
|---|---|---|
| ORF Start: at 1 | | ORF Stop: TAA at 1588 |
| SEQ ID NO: 364 | 529 aa | MW at 58496.2kD |

| | |
|---|---|
| NOV35d, | HRSHMPGSLPLNAEACWPKDVGIVALEIYFPSQYVDQAELEKYDGVDAGKYTIGLGQAKMGFCTDREDI |
| 253174237 | |
| Protein | NSLCMTVVQNLMERNNLSYDCIGRLEVGTETIIDKSKSVKTNLMQLFEESGNTDIEGIDTTNACYGGTA |
| Sequence | |
| | AVFNAVNWIESSSWDGRYALVVAGDIAVYATGNARPTGGVGAVALLIGPNAPLIFERGLRGTHMQHAYD |
| | FYKPDMLSEYPIVDGKLSIQCYLSALDRCYSVYCKKIHAQWQKEGNDKDFTLNDFGFMIFHSPYCKLVQ |
| | KSLARMLLNDFLNDQNRDKNSIYSGLEAFGDVKLEDTYFDRDVEKAFMKASSELFSQKTKASLLVSNQN |
| | GNMYTSSVYGSLASVLAQYSPQQLAGKRIGVFSYGSGLAATLYSLKVTQDATPGSALDKITASLCDLKS |
| | RLDSRTGVAPDVFAENMKLREDTHHLVNYIPQGSIDSLFEGTWYLVRVDEKHRRTYARRPTPNDDTLDE |
| | GVGLVHSNIATEHIPSPAKKVPRLPATAAEPEAAVISNGEHHHHHH |

| | |
|---|---|
| SEQ ID NO: 365 | 1608 bp |

| | |
|---|---|
| NOV35e, | CCTGGATCACTTCCTTTGAATGCAGAAGCTTGCTGGCCAAAAGATGTGCGAATTCTTCCCCTTGAGATC |
| 256420363 | |
| DNA | TATTTTCCTTCTCAATATGTTGATCAAGCAGAGTTGGAAAAATATGATGGTGTAGATGCTGGAAAGTAT |
| Sequence | |
| | ACCATTGGCTTGGGCCAGGCCAAGATGGGCTTCTGCACAGATAGAGAAGATATTAACTCTCTTTGCATG |
| | ACTGTGGTTCAGAATCTTATGGAGAGAAATAACCTTTCCTATGATTGCATTGGGCGGCTGGAAGTTGGA |
| | ACAGAGACAATCATCGACAAATCAAAGTCTGTGAAGACTAATTTGATGCAGCTGTTTGAAGAGTCTGGG |
| | AATACAGATATAGAAGGAATCGACACAACTAATGCATGCTATGGAGGCACAGCTGCTGTCTTCAATGCT |
| | GTTAACTGGATTGAGTCCAGCTCTTGGGATGGACGGTATGCCCTGGTAGTTGCAGGAGATATTGCTGTA |
| | TATGCCACAGGAAATGCTAGACCTACAGGTGGAGTTGGAGCAGTAGCTCTGCTAATTGGGCCAAATGCT |
| | CCTTTAATTTTTGAACGAGGGCTTCGTGGGACACATATGCAACATGCCTATGATTTTTACAAGCCTGAT |
| | ATGCTATCTGAATATCCTATAGTAGATGGAAAACTCTCCATACAGTGCTACCTCAGTGCATTAGACCGC |
| | TGCTACTCTGTCTACTGCAAAAAGATCCATGCCCAGTGGCAGAAAGAGGGAAATGATAAAGATTTTACC |
| | TTGAATGATTTTGGCTTCATGATCTTTCACTCACCATATTGTAAACTGGTTCAGAAATCTCTAGCTCGG |
| | ATGTTGCTGAATGACTTCCTTAATGACCAGAATAGAGATAAAAATAGTATCTATAGTGGCCTGGAAGCC |
| | TTTGGGGATGTTAAATTAGAAGACACCTACTTTGATAGAGATGTGGAGAAGGCATTTATGAAGGCTAGC |
| | TCTGAACTCTTCAGTCAGAAAACAAAGGCATCTTTACTTGTATCAAATCAAAATGGAAATATGTACACA |
| | TCTTCAGTATATGGTTCCCTTGCATCTGTTCTAGCACAGTACTCACCTCAGCAATTAGCAGGGAAGAGA |
| | ATTGGAGTGTTTTCTTATGGTTCTGGTTTGGCTGCCACTCTGTACTCTCTTAAAGTCACACAAGATGCT |
| | ACACCGGGGTCTGCTCTTGATAAAATAACAGCAAGTTTATGTGATCTTAAATCAAGGCTTGATTCAAGA |
| | ACTGGTGTGGCACCAGATGTCTTCGCTGAAAACATGAAGCTCAGAGAGGACACCCATCATTTGGTCAAC |
| | TATATTCCCCAGGGTTCAATAGATTCACTCTTTGAAGGAACGTCGTACTTAGTTAGGGTGGATGAAAAG |
| | CACAGAAGAACTTACGCTCGGCGTCCCACTCCAAATGATGACACTTTGGATGAAGGAGTAGGACTTGTG |
| | CATTCAAACATAGCAACTGAGCATATTCCAAGCCCTGCCAAGAAAGTACCAAGACTCCCTGCCACAGCA |
| | GCAGAACCTGAAGCAGCTGTCATTAGTAATGGGGAACATCATCACCACCATCACTAAGCGGCCGCACTC |
| | GAGCACCACCACCACCACCAC |

| | | |
|---|---|---|
| ORF Start: at 1 | | ORF Stop: TAA at 1573 |
| SEQ ID NO: 366 | 524 aa | MW at 57847.5kD |

| | |
|---|---|
| NOV35e, | PGSLPLNAEACWPKDVGIVALEIYFPSQYVDQAELEKYDGVDAGKYTIGLGQAKMGFCTDREDINSLCM |
| 256420363 | |
| Protein | TVVQNLMERNNLSYDCIGRLEVGTETIIDKSKSVKTNLMQLFEESGNTDIEGIDTTNACYGGTAAVFNA |
| Sequence | |
| | VNWIESSSWDGRYALVVAGDIAVYATGNARPTGGVGAVALLIGPNAPLIFERGLRGTHMQHAYDFYKPD |
| | MLSEYPIVDGKLSIQCYLSALDRCYSVYCKKIHAQWQKEGNDKDFTLNDFGFMIFHSPYCKLVQKSLAR |
| | MLLNDFLNDQNRDKNSIYSGLEAFGDVKLEDTYFDRDVEKAFMKASSELFSQKTKASLLVSNQNGNMYT |
| | SSVYGSLASVLAQYSPQQLAGKRIGVFSYGSGLAATLYSLKVTQDATPGSALDKITASLCDLKSRLDSR |
| | TGVAPDVFAENMKLREDTHHLVNYIPQGSIDSLFEGTWYLVRVDEKHRRTYARRPTPNDDTLDEGVGLV |
| | HSNIATEHIPSPAKKVPRLPATAAEPEAAVISNGEHHHHHH |

| | |
|---|---|
| SEQ ID NO: 367 | 1612 bp |

| | |
|---|---|
| NOV35f, | ACATCATCACCACCATCACCCTGGATCACTTCCTTTGAATGCAGAAGCTTGCTGGCCAAAAGATGTGGG |
| 255667064 | |
| DNA | AATTGTTGCCCTTGAGATCTATTTTCCTTCTCAATATGTTGATCAAGCAGAGTTGGAAAAATATGATGG |
| Sequence | |
| | TGTAGATGCTGGAAAGTATACCATTGGCTTGGGCCAGGCCAAGATGGGCTTCTGCACAGATAGAGAAGA |
| | TATTAACTCTCTTTGCATGACTGTGGTTCAGAATCTTATGGAGAGAAATAACCTTTCCTATGATTGCAT |
| | TGGGCGGCTGGAAGTTGGAACAGAGACAATCATCGACAAATCAAAGTCTGTGAAGACTAATTTGATGCA |
| | GCTGTTTGAAGAGTCTGGGAATACAGATATAGAAGGAATCGACACAACTAATGCATGCTATGGAGGCAC |
| | AGCTGCTGTCTTCAATGCTGTTAACTGGATTGAGTCCAGCTCTTGGGATGGACGGTATGCCCTGGTAGT |
| | TGCAGGAGATATTGCTCTATATGCCACAGGAAATGCTAGACCTACAGGTGGAGTTGGAGCAGTAGCTCT |
| | GCTAATTGGGCCAAATGCTCCTTTAATTTTTGAACGAGGGCTTCGTGGGACACATATGCAACATGCCTA |
| | TGATTTTTACAAGCCTGATATGCTATCTGAATATCCTATAGTAGATGGAAAACTCTCCATACAGTGCTA |
| | CCTCAGTGCATTAGACCGCTGCTACTCTGTCTACTGCAAAAAGATCCATGCCCAGTGGCAGAAAGAGGG |
| | AAATGATAAAGATTTTACCTTGAATGATTTTGGCTTCATGATCTTTCACTCACCATATTGTAAACTGGT |
| | TCAGAAATCTCTAGCTCGGATGTTGCTGAATGACTTCCTTAATGACCAGAATAGAGATAAAAATAGTAT |
| | CTATAGTGGCCTGGAAGCCTTTGGGGATGTTAAATTAGAAGACACCTACTTTGATAGAGATGTGGAGAA |
| | GGCATTTATGAAGGCTAGCTCTGAACTCTTCAGTCAGAAAACAAAGGCATCTTTACTTGTATCAAATCA |
| | AAATGGAAATATGTACACATCTTCAGTATATGGTTCCCTTGCATCTGTTCTAGCACAGTACTCACCTCA |
| | GCAATTAGCAGGGAAGAGAATTGGAGTGTTTTCTTATGGTTCTGGTTTGGCTGCCACTCTGTACTCTCT |
| | TAAAGTCACACAAGATGCTACACCGGGGTCTGCTCTTGATAAAATAACAGCAAGTTTATGTGATCTTAA |
| | ATCAAGGCTTGATTCAAGAACTGGTGTGGCACCAGATGTCTTCGCTGAAAACATGAAGCTCAGAGAGGA |
| | CACCCATCATTTGGTCAACTATATTCCCCAGGGTTCAATAGATTCACTCTTTGAAGGAACGTGGTACTT |
| | AGTTAGGGTGGATGAAAAGCACAGAAGAACTTACGCTCGGCGTCCCACTCCAAATGATGACACTTTGGA |
| | TGAAGGAGTAGGACTTGTGCATTCAAACATAGCAACTGAGCATATTCCAAGCCCTGCCAAGAAAGTACC |
| | AAGACTCCCTGCCACAGCAGCAGAACCTGAAGCAGCTGTCATTAGTAATGGGGAACATTAAGCGGCCGC |
| | ACTCGAGCACCACCACCACCACCAC |

| | | |
|---|---|---|
| ORF Start: at 2 | | ORF Stop: TAA at 1577 |
| SEQ ID NO: 368 | 525 aa | MW at 57984.6kD |

| | |
|---|---|
| NOV35f, | HHHHHHPGSLPLNAEACWPKDVGIVALEIYFPSQYVDQAELEKYDGVDAGKYTIGLGQAKMGFCTDRED |
| 255667064 | |
| Protein | INSLCMTVVQNLMERNNLSYDCIGRLEVGTETIIDKSKSVKTNLMQLFEESGNTDIEGIDTTNACYGGT |
| Sequence | |
| | AAVFNAVNWIESSSWDGRYALVVAGDIAVYATGNARPTGGVGAVALLIGPNAPLIFERGLRGTHMQHAY |
| | DFYKPDMLSEYPIVDGKLSIQCYLSALDRCYSVYCKKIHAQWQKEGNDKDFTLNDFGFMIFHSPYCKLV |
| | QKSLARMLLNDFLNDQNRDKNSIYSGLEAFGDVKLEDTYFDRDVEKAFMKASSELFSQKTKASLLVSNQ |
| | NGNMYTSSVYGSLASVLAQYSPQQLAGKRIGVFSYGSGLAATLYSLKVTQDATPGSALDKITASLCDLK |
| | SRLDSRTGVAPDVFAENMKLREDTHHLVNYIPQGSIDSLEFGTWYLVRVDEKHRRTYARRPTPNDDTLD |
| | EGVGLVHSNIATEHIPSPAKKVPRLPATAAEPEAAVISNGEH |

| | |
|---|---|
| SEQ ID NO: 369 | 1564 bp |

| | |
|---|---|
| NOV35g, | CATGCCTGGATCACTTCCTTTGAATGCAGAAGCTTGCTGGCCCAAAAGATGTGGGAATTGTTGCCCTTGA |
| 228832739 | |
| DNA | GATCTATTTTCCTTCTCAATATGTTGATCAAGCAGAGTTGGAAAAATATGATGGTGTAGATGCTGGAAA |
| Sequence | |
| | GTATACCATTGGCTTGGGCCAGGCCAAGATGGGCTTCTGCACAGATAGAGAAGATATTAACTCTCTTTG |
| | CATGACTGTGGTTCAGAATCTTATGGAGAGAAATAACCTTTCCTATGATTGCATTGGGCGGCTGGAAGT |
| | TGGAACAGAGACAATCATCGACAAATCAAAGTCTGTGAAGACTAATTTGATGCAGCTGTTTGAAGAGTC |
| | TGGGAATACAGATATAGAAGGAATCGACACAACTAATGCATGCTATGGAGGCACAGCTGCTGTCTTCAA |
| | TGCTGTTAACTGGATTGAGTCCAGCTCTTGGGATGGACGGTATGCCCTGGTAGTTGCAGGAGATATTGC |
| | TGTATATGCCACAGGAAATGCTAGACCTACAGGTGGAGTTGGAGCAGTAGCTCTGCTAATTGGGCCAAA |
| | TGCTCCTTTAATTTTTGAACGAGGGCTTCGTGGGACACATATGCAACATGCCTATGATTTTTACAAGCC |
| | TGATATGCTATCTGAAATATCCTATAGTAGATGGAAACTCTCCATACAGTGCTACCTCAGTGCATTAGA |
| | CCGCTGCTACTCTGTCTACTGCAAAAAGATCCATGCCCAGTGGCAGAAAGAGGGAAATGATAAAGATTT |
| | TACCTTGAATGATTTTGGCTTCATGATCTTTCACTCACCATATTGTAAACTGGTTCAGAAATCTCTAGC |
| | TCGGATGTTGCTGAATGACTTCCTTAATGACCAGAATAGAGATAAAAATAGTATCTATAGTGGCCTGGA |
| | AGCCTTTGGGGATGTTAAATTAGAAGACACCTACTTTGATAGAGATGTGGAGAAGGCATTTATGAAGGC |
| | TAGCTCTGAACTCTTCAGTCAGAAAACAAAGGCATCTTTACTTGTATCAAATCAAAATGGAAATATGTA |
| | CACATCTTCAGTATATGGTTCCCTTGCATCTGTTCTAGCACAGTACTCACCTCAGCAATTAGCAGGGAA |
| | GAGAATTGGAGTGTTTTCTTATGGTTCTGGTTTGGCTGCCACTCTGTACTCTCTTAAAGTCACACAAGA |
| | TGCTACACCGGGGTCTGCTCTTGATAAAATAACAGCAAGTTTATGTGATCTTAAATCAAGGCTTGATTC |
| | AAGAACTGGTGTGGCACCAGATGTCTTCGCTGAAAACATGAAGCTCAGAGAGGACACCCATCATTTGGT |
| | CAACTATATTCCCCAGGGTTCAATAGATTCACTCTTTGAAGGAACGTGGTACTTAGTTAGGGTGGATGA |
| | AAAGCACAGAAGAACTTACGCTCGGCGTCCCACTCCAAATGATGACACTTTGGATGAAGGAGTAGGACT |
| | TGTGCATTCAAACATAGCAACTGAGCATATTCCAAGCCCTGCCAAGAAAGTACCAAGACTCCCTGCCAC |
| | AGCAGCAGAACCTGAAGCAGCTGTCATTAGTAATGGGGAACATTAA |

| | | |
|---|---|---|
| ORF Start: ATG at 2 | | ORF Stop: TAA at 1562 |
| SEQ ID NO: 370 | 520 aa | MW at 57293.0kD |

| | |
|---|---|
| NOV35g, | MPGSLPLNAEACWPKDVGIVALEIYFPSQYVDQAELEKYDGVDAGKYTIGLGQAKMGFCTDREDINSLC |
| 228832739 | |
| Protein | MTVVQNLMERNNLSYDCIGRLEVGTETIIDKSKSVKTNLMQLFEESGNTDIEGIDTTNACYGGTAAVFN |
| Sequence | |
| | AVNWIESSSWDGRYALVVAGDIAVYATGNARPTGGVGAVALLIGPNAPLIFERGLRGTHMQHAYDFYKP |
| | DMLSEYPIVDGKLSIQCYLSALDRCYSVYCKKIHAQWQKEGNDKDFTLNDFGFMIFHSPYCKLVQKSLA |
| | RMLLNDFLNDQNRDKNSIYSGLEAFGDVKLEDTYFDRDVEKAFMKASSELFSQKTKASLLVSNQNGNMY |
| | TSSVYGSLASVLAQYSPQQLAGKRIGVFSYGSGLAATLYSLKVTQDATPGSALDKITASLCDLKSRLDS |
| | RTGVAPDVFAENMKLREDTHHLVNYIPQGSIDSLFEGTWYLVRVDEKHRRTYARRPTPNDDTLDEGVGL |
| | VHSNIATEHIPSPAKKVPRLPATAAEPEAAVISNGEH |

| | |
|---|---|
| SEQ ID NO: 371 | 1650 bp |

| | |
|---|---|
| NOV35h, | CCTTCACACAGCTCTTTCACCATGCCTGGATCACTTCCTTTGAATGCAGAAGCTTGCTGGCCAAAAGAT |
| CG97025-02 | |
| DNA | GTTGGGATTGTTGCCCTTGAGATCTATTTTCCTTCTCAATATGTTGATCAAGCAGAGTTGGAAAAATAT |
| Sequence | |
| | GATGGTGTAGATGCTGGGAAGTATACCATTGGCTTGGGCCAGGCCAAGATGGGCTTCTGCACAGATAGA |
| | GAAGATATTAACTCTCTTTGCATGACTGTGGTTCAGAATCTTATGGAGAGAAATAACCTTTCCTATGAT |
| | TGCATTGGGCGGCTGGAAGTTGGAACAGAGACAATCATCGACAAATCAAAGTCTGTGAAGACTAATTTG |
| | ATGCAGCTGTTTGAAGAGTCTGGGAATACAGATATAGAAGGAATCGACACAACTAATGCATGCTATGGA |
| | GGCACAGCTGCTGTCTTCAATGCTGTTAACTGGATTGAGTCCAGCTCTTGGGATGGACGGTATGCCCTG |
| | GTAGTTGCAGGACATATTGCTGTATATGCCACAGGAAATGCTAGACCTACAGGTGGAGTTGGAGCAGTA |
| | GCTCTGCTAATTGGGCCAAATGCTCCTTTAATTTTTGAACGAGGGCTTCGTGGGACACATATGCAACAT |
| | GCCTATGATTTTTACAAGCCTGATATGCTATCTGAATATCCTATAGTAGATGGGAAACTCTCCATACAG |
| | TGCTACCTCAGTGCATTAGACCGCTGCTATTCTGTCTACTGCAAAAAGATCCATGCCCAGTGGCAGAAA |
| | GAGGGAAATGATAAAGATTTTACCTTGAATGATTTTGGCTTCATGATCTTTCACTCACCATATTGTAAA |
| | CTGGTTCAGAAATCTCTAGCTCGGATGTTGCTGAATGACTTCCTTAATGACCAGAATAGAGATAAAAAT |
| | AGTATCTATAGTGGCCTGGAAGCCTTTGGGGATGTTAAATTAGAAGACACCTACTTTGATAGAGATGTG |
| | GAGAAGGCATTTATGAAGGCTAGCTCTGAACTCTTCAGTCAGAAAACAAAGGCATCTTTACTTGTATCA |
| | AATCAAAATGGAAATATGTACACATCTTCAGTATATGGTTCCCTTGCATCTGTTCTAGCACAGTACTCA |
| | CCTCAGCAATTAGCAGGGAAGAGAATTGGAGTGTTTTCTTATGGTTCTGGTTTGGCTGCCACTCTGTAC |
| | TCTCTTAAAGTCACACAAGATGCTACACCGGGGTCTGCTCTTGATAAAATAACAGCAAGTTTATGTGAT |
| | CTTAAATCAAGGCTTGATTCAAGAACTGGTGTGGCACCAGATGTCTTCGCTGAAAACATGAAGCTCAGA |
| | GAGGACACCCATCATTTGGTCAACTATATTCCCCAGGGTTCAATAGATTCACTCTTTGAAGGAACGTGG |
| | TACTTAGTTAGGGTGGATGAAAAGCACAGAAGAACTTACGCTCGGCGTCCCACTCCAAATGATGACACT |
| | TTGGATGAAGGAGTAGGACTTGTGCATTCAAACATAGCAACTGAGCATATTCCAAGCCCTGCCAAGAAA |
| | GTACCAAGACTCCCTGCCACAGCAGCAGAACCTGAAGCAGCTGTCATTAGTAATGGGGAACATTAAGAT |
| | ACTCTGTGAGGTGCAAGACTTCAGGGTGGGGTGGGCATGGGGTGGGGGTATGGGAACAGTTGG |

| | | |
|---|---|---|
| ORF Start: ATG at 22 | | ORF Stop: TAA at 1582 |
| SEQ ID NO: 372 | 520 aa | MW at 57293.0kD |

| | |
|---|---|
| NOV35h, | MPGSLPLNAEACWPKDVGIVALEIYFPSQYVDQAELEKYDGVDAGKYTIGLGQAKMGFCTDREDINSLC |
| CG97025-02 | |
| Protein | MTVVQNLMERNNLSYDCIGRLEVGTETIIDKSKSVKTNLMQLFEESGNTDIEGIDTTNACYGGTAAVFN |
| Sequence | |
| | AVNWIESSSWDGRYALVVAGDIAVYATGNARPTGGVGAVALLIGPNAPLIFERGLRGTHMQHAYDFYKP |
| | DMLSEYPIVDGKLSIQCYLSALDRCYSVYCKKIHAQWQKEGNDKDFTLNDFGFMIFHSPYCKLVQKSLA |
| | RMLLNDFLNDQNRDKNSIYSGLEAFGDVKLEDTYFDRDVEKAFMKASSELFSQKTKASLLVSNQNCHNY |
| | TSSVYGSLASVLAQYSPQQLAGKRIGVFSYGSGLAATLYSLKVTQDATPGSALDKITASLCDLKSRLDS |
| | RTGVAPDVFAENMKLREDTHHLVNYIPQGSIDSLFEGTWYLVRVDEKHRRTYARRPTPNDDTLDEGVGL |
| | VHSNIATEHIPSPAKKVPRLPATAAEPEAAVISNGEH |

| | |
|---|---|
| SEQ ID NO:373 | 1564 bp |

| | |
|---|---|
| NOV35i, | CATGCCTGGATCACTTCCTTTGAATGCAGAAGCTTGCTGGCCAAAAGATGTGGGAATTGTTGCCCTTGA |
| CG97025-03 | |
| DNA | GATCTATTTTCCTTCTCAATATGTTGATCAAGCAGAGTTGGAAAAATATGATGGTGTAGATGCTGGAAA |
| Sequence | |
| | GTATACCATTGGCTTGGGCCAGGCCAAGATGGGCTTCTGCACAGATAGAGAAGATATTAACTCTCTTTG |
| | CATGACTGTGGTTCAGAATCTTATGGAGAGAAATAACCTTTCCTATGATTGCATTGGGCGGCTGGAAGT |
| | TGGAACAGAGACAATCATCGACAAATCAAAGTCTGTGAAGACTAATTTGATGCAGCTGTTTGAAGAGTC |
| | TGGGAATACAGATATAGAAGGAATCGACACAACTAATGCATGCTATGGAGGCACAGCTGCTGTCTTCAA |
| | TGCTGTTAACTGGATTGAGTCCAGCTCTTGGGATGGACGGTATGCCCTGGTAGTTGCAGGAGATATTGC |
| | TGTATATGCCACAGGAAATGCTAGACCTACAGGTGGAGTTGGAGCAGTAGCTCTGCTAATTGGGCCAAA |
| | TGCTCCTTTAATTTTTGAACGAGGGCTTCGTGGGACACATATGCAACATGCCTATGATTTTTACAAGCC |
| | TGATATGCTATCTGAATATCCTATAGTAGATGGAAAACTCTCCATACAGTGCTACCTCAGTGCATTAGA |
| | CCGCTGCTACTCTGTCTACTGCAAAAAGATCCATGCCCAGTGGCAGAAAGAGGGAAATGATAAAGATTT |
| | TACCTTGAATGATTTTGGCTTCATGATCTTTCACTCACCATATTGTAAACTGGTTCAGAAATCTCTAGC |
| | TCGGATGTTGCTGAATGACTTCCTTAATGACCAGAATAGAGATAAAAATAGTATCTATAGTGGCCTGGA |
| | AGCCTTTGGGGATGTTAAATTAGAAGACACCTACTTTGATAGAGATGTGGAGAAGGCATTTATGAAGGC |
| | TAGCTCTGAACTCTTCAGTCAGAAAACAAAGGCATCTTTACTTGTATCAAATCAAAATGGAAATATGTA |
| | CACATCTTCAGTATATGGTTCCCTTGCATCTGTTCTAGCACAGTACTCACCTCAGCAATTAGCAGGGAA |
| | GAGAATTGGAGTGTTTTCTTATGGTTCTGGTTTGGCTGCCACTCTGTACTCTCTTAAAGTCACACAAGA |
| | TGCTACACCGGGGTCTGCTCTTGATAAAATAACAGCAAGTTTATGTGATCTTAAATCAAGGCTTGATTC |
| | AAGAACTGGTGTGGCACCAGATGTCTTCGCTGAAAACATGAAGCTCAGAGAGGACACCCATCATTTGGT |
| | CAACTATATTCCCCAGGGTTCAATAGATTCACTCTTTGAAGGAACGTGGTACTTAGTTAGGGTGGATGA |
| | AAAGCACAGAAGAACTTACGCTCGGCGTCCCACTCCAAATGATGACACTTTGGATGAAGGAGTAGGACT |
| | TGTGCATTCAAACATAGCAACTGAGCATATTCCAAGCCCTGCCAAGAAAGTACCAAGACTCCCTGCCAC |
| | AGCAGCAGAACCTGAAGCAGCTGTCATTAGTAATGGGGAACATTAA |

| | | |
|---|---|---|
| ORF Start: ATG at 2 | | ORF Stop: TAA at 1562 |
| SEQ ID NO: 374 | 520 aa | MW at 57293.0kD |

| | |
|---|---|
| NOV35i | MPGSLPLNAEACWPKDVGIVALEIYFPSQYVDQAELEKYDGVDAGKYTIGLGQAKMGFCTDREDINSLC |
| CG97025-03 | |
| Protein | MTVVQNLMERNNLSYDCIGRLEVGTETIIDKSKSVKTNLMQLFEESGNTDIEGIDTTNACYGGTAAVFN |
| Sequence | |
| | AVNWIESSSWDGRYALVVAGDIAVYATGNARPTGGVGAVALLIGPNAPLIFERGLRGTHMQHAYDFYKP |
| | DMLSEYPIVDGKLSIQCYLSALDRCYSVYCKKIHAQWQKEGNDKDFTLNDFGFMIFHSPYCKLVQKSLA |
| | RMLLNDFLNDQNRDKNSIYSGLEAFGDVKLEDTYFDRDVEKAFMKASSELFSQKTKASLLVSNQNGNMY |
| | TSSVYGSLASVLAQYSPQQLAGKRIGVFSYGSGLAATLYSLKVTQDATPGSALDKITASLCDLKSRLDS |
| | RTGVAPDVFAENMKLREDTHHLVNYIPQGSIDSLEFGTWYLVRVDEKHRRTYARRPTPNDDTLDEGVGL |
| | VHSNIATEHIPSPAKKVPRLPATAAEPEAAVISNGEH |

| | |
|---|---|
| SEQ ID NO:375 | 1608 bp |

| | |
|---|---|
| NOV35j, | CCTGGATCACTTCCTTTGAATGCAGAAGCTTGCTGGCCAAAAGATGTGGGAATTGTTGCCCTTGAGATC |
| CG97025-05 | |
| DNA | TATTTTCCTTCTCAATATGTTGATCAAGCAGAGTTGGAAAAATATGATGGTGTAGATGCTGGAAAGTAT |
| Sequence | |
| | ACCATTGGCTTGGGCCAGGCCAAGATGGGCTTCTGCACAGATAGAGAAGATATTAACTCTCTTTGCATG |
| | ACTGTGGTTCAGAATCTTATGGAGAGAAATAACCTTTCCTATGATTGCATTGGGCGGCTGGAAGTTGGA |
| | ACAGAGACAATCATCGACAAATCAAAGTCTGTGAAGACTAATTTGATGCAGCTGTTTGAAGAGTCTGGG |
| | AATACAGATATAGAAGGAATCGACACAACTAATGCATGCTATGGAGGCACAGCTGCTGTCTTCAATGCT |
| | GTTAACTGGATTGAGTCCAGCTCTTGGGATGGACGGTATGCCCTGGTAGTTGCAGGAGATATTGCTGTA |
| | TATGCCACAGGAAATGCTAGACCTACAGGTGGAGTTGGAGCAGTAGCTCTGCTAATTGGGCCAAATGCT |
| | CCTTTAATTTTTGAACGAGGGCTTCGTGGGACACATATGCAACATGCCTATGATTTTTACAAGCCTGAT |
| | ATGCTATCTGAATATCCTATAGTAGATGGAAAACTCTCCATACAGTGCTACCTCAGTGCATTAGACCGC |
| | TGCTACTCTGTCTACTGCAAAAAGATCCATGCCCAGTGGCAGAAAGAGGGAAATGATAAAGATTTTACC |
| | TTGAATGATTTTGCCTTCATGATCTTTCACTCACCATATTGTAAACTGGTTCAGAAATCTCTAGCTCCG |
| | ATGTTGCTGAATGACTTCCTTAATGACCAGAATAGAGATAAAAATAGTATCTATAGTGGCCTGGAAGCC |
| | TTTGGGGATGTTAAATTAGAAGACACCTACTTTGATAGAGATGTGGAGAAGGCATTTATGAAGGCTAGC |
| | TCTGAACTCTTCAGTCAGAAAACAAAGGCATCTTTACTTGTATCAAATCAAAATGGAAATATGTACACA |
| | TCTTCAGTATATGGTTCCCTTGCATCTGTTCTAGCACAGTACTCACCTCAGCAATTAGCAGGGAAGAGA |
| | ATTGGAGTGTTTTCTTATGGTTCTGGTTTGGCTGCCACTCTGTACTCTCTTAAAGTCACACAAGATGCT |
| | ACACCGGGGTCTGCTCTTGATAAAATAACAGCAAGTTTATGTGATCTTAAATCAAGGCTTGATTCAAGA |
| | ACTGGTGTGGCACCAGATGTCTTCGCTGAAAACATGAAGCTCACAGAOGACACCCATCATTTGGTCAAC |
| | TATATTCCCCAGGGTTCAATACATTCACTCTTTGAAGGAACGTGGTACTTAGTTAGGGTGGATGAAAAG |
| | CACAGAAGAACTTACGCTCGGCGTCCCACTCCAAATGATGACACTTTGGATGAAGGAGTAGGACTTGTG |
| | CATTCAAACATAGCAACTGAGCATATTCCAAGCCCTGCCAAGAAAGTACCAAGACTCCCTGCCACAGCA |
| | GCAGAACCTGAAGCAGCTGTCATTAGTAATGGGGAACATCATCACCACCATCACTAAGCGGCCGCACTC |
| | GACCACCACCACCACCACCAC |

| | | |
|---|---|---|
| ORF Start: at 1 | | ORF Stop: TAA at 1573 |
| SEQ ID NO: 376 | 524 aa | MW at 57847.5kD |

| | |
|---|---|
| NOV35j, | PGSLPLNAEACWPKDVGIVALEIYPPSQYVDQAELEKYDGVDACKYTIGLGQAKMGFCTDREDINSLCM |
| CG97025-05 | |
| Protein | TVVQNLMERNNLSYDCIGRLEVGTETIIDKSKSVKTNLMQLFEESGNTDIEGIDTTNACYGGTAAVFNA |
| Sequence | |
| | VNWIESSSWDGRYALVVAGDIAVYATGNARPTGGVGAVALLIGPNAPLIFERGLRGTHMQHAYDPYKPD |
| | MLSEYPIVDGKLSIQCYLSALDRCYSVYCKKIHAQWQKSGNOKDFTLNDPGFMIFHSPYCKLVQKSLAR |
| | MLLNDFLNDQNRDKNSIYSGLEAFGDVKLEDTYFDRDVEKAFMKASSELFSQKTKASLLVSNQNQNMYT |
| | SSVYGSLASVLAQYSPQQLAGKRIGVFSYGSGLAATLYSLKVTQDATPGSALDKITASLCDLKSRLDSR |
| | TGVAPDVFAENMKLREDTHHLVNYIPQGSIDSLFEGTWYLVRVDEKHRRTYARRPTPNDDTLDEGVGLV |
| | HSNIATEHIPSPAKKVPRLPATAAEPEAAVISNGEHHHHH |

Sequence comparison of the above protein sequences yields the following sequence relationships shown in Table 35B.

TABLE 35B
Comparison of NOV35a against NOV35b through NOV35j.

| | | Identities/ |
|---|---|---|
| | NOV35a Residues/ | Similarities for the |
| Protein Sequence | Match Residues | Matched Region |
| NOV35b | 7 . . . 525 | 519/519 (100%) |
| | 2 . . . 520 | 519/519 (100%) |
| NOV35c | 7 . . . 525 | 519/519 (100%) |
| | 1 . . . 519 | 519/519 (100%) |
| NOV35d | 2 . . . 525 | 521/524 (99%) |
| | 1 . . . 524 | 521/524 (99%) |
| NOV35e | 7 . . . 525 | 519/519 (100%) |
| | 1 . . . 519 | 519/519 (100%) |
| NOV35f | 1 . . . 525 | 525/525 (100%) |
| | 1 . . . 525 | 525/525 (100%) |
| NOV35g | 7 . . . 525 | 519/519 (100%) |
| | 2 . . . 520 | 519/519 (100%) |
| NOV35h | 7 . . . 525 | 519/519 (100%) |
| | 2 . . . 520 | 519/519 (100%) |
| NOV35i | 7 . . . 525 | 519/519 (100%) |
| | 2 . . . 520 | 519/519 (100%) |
| NOV35j | 7 . . . 525 | 519/519 (100%) |
| | 1 . . . 519 | 519/519 (100%) |

Further analysis of the NOV35a protein yielded the following properties shown in Table 35C.

TABLE 35C
Protein Sequence Properties NOV35a

| | |
|---|---|
| SignalP analysis: | No Known Signal Sequence Predicted |
| PSORT II analysis: | PSG: a new signal peptide prediction method |
| | N-region: length 0; pos.chg 0; neg.chg 0 |
| | H-region: length 14; peak value 1.89 |
| | PSG score: −2.51 |
| | GvH: von Heijne's method for signal seq. recognition |
| | GvH score (threshold: −2.1): −9.72 |
| | possible cleavage site: between 19 and 20 |
| | >>> Seems to have no N-terminal signal peptide |
| | ALOM: Klein et al's method for TM region allocation |
| | Init position for calculation: 1 |
| | Tentative number of TMS(s) for the threshold 0.5: 1 |
| | Number of TMS(s) for threshold 0.5: 0 |
| | PERIPHERAL Likelihood = 3.87 (at 375) |
| | ALOM score: −1.17 (number of TMSs: 0) |
| | MITDISC: discrimination of mitochondrial targeting seq |

| | | | |
|---|---|---|---|
| R content: | 0 | Hyd Moment(75): | 2.33 |
| Hyd Moment(95): | 2.24 | G content: | 1 |
| D/E content: | 2 | S/T content: | 1 |
| Score: | −8.20 | | |

Gavel: prediction of cleavage sites for mitochondrial preseq
cleavage site motif not found
NUCDISC: discrimination of nuclear localization signals
pat4: KHRR (3) at 466
pat7: none
bipartite: none
content of basic residues: 9.5%
NLS Score: −0.29
KDEL: ER retention motif in the C-terminus: none
ER Membrane Retention Signals: none
SKL: peroxisomal targeting signal in the C-terminus: none
PTS2: 2nd peroxisomal targeting signal: found
KLREDTHHL at 433
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
47.8%: cytoplasmic
34.8%: nuclear
17.4%: mitochondrial
>> prediction for CG97025-04 is cyt (k = 23)

A search of the NOV35a protein against the Geneseq database, a proprietary database that contains sequences published in patents and patent publication, yielded several homologous proteins shown in Table 35D.

TABLE 35D
Geneseq Results for NOV35a

| Geneseq | Protein/Organism/Length | NOV35a Residues/ | Identities Similarities | Expect |
|---|---|---|---|---|
| Identifier | [Patent #, Date] | Match Residues | for the Matched Region | Value |
| AAW32222 | Avian | 7 . . . 525 | 437/521 (83%) | 0.0 |
| | 3-hydroxy-2-methylglutaryl-CoA | 2 . . . 522 | 475/521 (90%) | |
| | synthase - Aves, 522 aa. | | | |
| | [US5668001-A, 16-SEP-1997] | | | |
| AAM79853 | Human protein SEQ ID NO 3499 - | 1 . . . 475 | 316/475 (66%) | 0.0 |
| | Homo sapiens, 518 aa. | 43 . . . 517 | 388/475 (81%) | |
| | [WO200157190-A2, 09-AUG-2001] | | | |
| AAM78869 | Human protein SEQ ID NO 1531 - | 1 . . . 475 | 316/475 (66%) | 0.0 |
| | Homo sapiens, 508 aa. | 33 . . . 507 | 388/475 (81%) | |
| | [WO200157190-A2, 09-AUG-2001] | | | |
| ABB66034 | Drosophila melanogaster polypeptide | 18 . . . 476 | 294/459 (64%) | e−170 |
| | SEQ ID NO 24894 - Drosophila | 5 . . . 459 | 353/459 (76%) | |
| | melanogaster, 465 aa. | | | |
| | [WO200171042-A2, 27-SEP-2001] | | | |
| ABB60545 | Drosophila melanogaster polypeptide | 18 . . . 476 | 294/459 (64%) | e−170 |
| | SEQ ID NO 8427 - Drosophila | 5 . . . 459 | 353/459 (76%) | |
| | melanogaster, 465 aa. | | | |
| | [WO200171042-A2, 27-SEP-2001] | | | |

In a BLAST search of public sequence datbases, the NOV35a protein was found to have homology to the proteins shown in the BLASTP data in Table 35E.

TABLE 35E
Public BLASTP Results for NOV35a

| Protein | | | | |
|---|---|---|---|---|
| Accession | | NOV35a Residues/ | Identities/Similarities | Expect |
| Number | Protein/Organism/Length | Match Residues | for the Matched Portion | Value |
| Q01581 | Hydroxymethylglutaryl-CoA synthase, | 7 . . . 525 | 519/519 (100%) | 0.0 |
| | cytoplasmic (EC 4.1.3.5) (HMG-CoA | 2 . . . 520 | 519/519 (100%) | |
| | synthase) (3-hydroxy-3-methylglutaryl | | | |
| | coenzyme A synthase) - Homo sapiens | | | |
| | (Human), 520 aa | | | |
| S27197 | hydroxymethylglutaryl-CoA synthase | 7 . . . 523 | 512/517 (99%) | 0.0 |
| | (EC 4.1.3.5), cytosolic, fibroblast | 2 . . . 518 | 513/517 (99%) | |
| | isoform - human, 520 aa. | | | |
| Q8N995 | Hypothetical protein FLJ38173 - | 7 . . . 525 | 508/519 (97%) | 0.0 |
| | Homo sapiens (Human), 509 aa. | 2 . . . 509 | 508/519 (97%) | |
| P17425 | Hydroxymethylglutaryl-CoA synthase, | 7 . . . 525 | 492/519 (94%) | 0.0 |
| | cytoplasmic (EC 4.1.3.5) (HMG-CoA | 2 . . . 520 | 507/519 (96%) | |
| | synthase) (3-hydroxy-3-methylglutaryl | | | |
| | coenzyme A synthase) - Rattus | | | |
| | norvegicus (Rat), 520 aa. | | | |
| P13704 | Hydroxymethylglutaryl-CoA synthase, | 7 . . . 525 | 494/519 (95%) | 0.0 |
| | cytoplasmic (EC 4.1.3.5) (HMG-CoA | 2 . . . 520 | 505/519 (97%) | |
| | synthase) (3-hydroxy-3-methylglutaryl | | | |
| | coenzyme A synthase) - Cricetulus | | | |
| | griseus (Chinese hamster), 520 aa. | | | |

PFam analysis predicts that the NOV35a protein contains the domains shown in the Table 35F.

TABLE 35F
Domain Analysis of NOV35a

| | NOV35a | Identities/Similarities | Expect |
|---|---|---|---|
| Pfam Domain | Match Region | for the Matched Region | Value |
| HMG_CoA_synt | 18 . . . 474 | 334/461 (72%) | 0 |
| | | 434/461 (94%) | |

### Example B: Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C: Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune/autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4, v1.5 and v1.6

The plates for Panels 1.4, v1.5 and v1.6 include two control wells (genomic DNA control and chemistry control) and 94 wells containing cDNA from various samples. The samples in Panels 1.4, v1.5 and v1.6 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, v1.5 and v1.6 are widely available through the American Type Culture Collection (ATCC), a repository for cultured cell lines, and were cultured using the conditions recommended by the ATCC. The normal tissues found on Panels 1.4, v1.5 and v1.6 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include two control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics. The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen. General oncology screening panel_v_{—}2.4 is an updated version of Panel 2D.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

Panels 3D and 3.1

The plates of Panels 3D and 3.1 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Oncology_cell_line_screening_panel_v3.2 is an updated version of Panel 3. The cell lines in panel 3D, 3.1, 1.3D and oncology_cell_line_screening_panel_v3.2 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI>30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures.

All samples were processed at CuraGen to produce single stranded cDNA. RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG101719-04 and CG101719-05: Fibroblast Growth Factor Receptor 1 IIIb-Like Protein.

Expression of gene CG101719-04 was assessed using the primer-probe sets Ag4049 and Ag5848, described in Tables AA and AB. Results of the RTQ-PCR runs are shown in Tables AC, AD, AE, AF, AG, AH, AI, AJ and AK.

TABLE AA

Probe Name Ag4049

| | | | Start | SEQ ID |
|---|---|---|---|---|
| Primers | | Length | Position | No |
| Forward | 5′-gccaagacagtgaagttcaaat-3′ | 22 | 626 | 482 |
| Probe | TET-5′-agtgggaccccaaaccccacact-3′-TAMRA | 23 | 656 | 483 |
| Reverse | 5′-aggtttgaattctttgccattt-3′ | 22 | 691 | 484 |

TABLE AB

Probe Name Ag5848

| | | | | |
|---|---|---|---|---|
| | | | Start | SEQ ID |
| Primers | Sequence | Length | Position | No |

| | | | | |
|---|---|---|---|---|
| Forward | 5′-ctaaagcacatcgaggtgaatg-3′ | 22 | 983 | 485 |
| Probe | TET-5′-agattggcccagacaacctgccttat-3′-TAMRA | 26 | 1011 | 486 |
| Reverse | 5′-agctattaatccccgaatgct-3′ | 21 | 1050 | 487 |

TABLE AC
AI.05 chondrosarcoma

| | Rel. Exp.(%) |
|---|---|
| | Ag5848, Run |
| Tissue Name | 306518773 |
| 138353_PMA (18hrs) | 21.5 |
| 138352_IL-1beta + Oncostatin M | 41.8 |
| 138351_IL-1beta+TNFa (18hrs) | 93.3 |
| 138350_IL-1beta (18hrs) | 86.5 |
| 138354_Untreated-complete | 7.7 |
| medium (18hrs) | |
| 138347_PMA (6hrs) | 39.8 |
| 138346_IL-1beta + Oncostatin M | 76.3 |
| 138345_IL-1beta+TNFa (6hrs) | 62.0 |
| 138344_IL-1beta (6hrs) | 40.3 |
| 138348_Untreated-complete | 36.1 |
| medium (6hrs) | |
| 138349_Untreated-serum starved | 100.0 |
| (6hrs) | |

TABLE AD
AI_comprehensive_panel_v1.0

| | Rel. | Rel. |
|---|---|---|
| | Exp. (%) | Exp. (%) |
| | Ag4049, | Ag5848, |
| | Run | Run |
| Tissue Name | 257315370 | 257315389 |
| 110967 COPD-F | 27.7 | 2.2 |
| 110980 COPD-F | 17.2 | 1.0 |
| 110968 COPD-M | 14.1 | 0.0 |
| 110977 COPD-M | 44.4 | 6.3 |
| 110989 Emphysema-F | 22.1 | 6.8 |
| 110992 Emphysema-F | 7.2 | 3.7 |
| 110993 Emphysema-F | 21.6 | 0.9 |
| 110994 Emphysema-F | 12.7 | 2.3 |
| 110995 Emphysema-F | 15.5 | 5.8 |
| 110996 Emphysema-F | 3.2 | 1.1 |
| 110997 Asthma-M | 6.7 | 1.0 |
| 111001 Asthma-F | 17.9 | 0.0 |
| 111002 Asthma-F | 16.7 | 5.6 |
| 111003 Atopic Asthma-F | 12.7 | 0.0 |
| 111004 Atopic Asthma-F | 13.0 | 0.0 |
| 111005 Atopic Asthma-F | 7.9 | 1.1 |
| 111006 Atopic Asthma-F | 2.4 | 0.0 |
| 111417 Allergy-M | 9.7 | 1.5 |
| 112347 Allergy-M | 0.4 | 0.0 |
| 112349 Normal Lung-F | 0.2 | 0.0 |
| 112357 Normal Lung-F | 28.9 | 5.4 |
| 112354 Normal Lung-M | 12.9 | 1.6 |
| 112374 Crohns-F | 31.9 | 5.3 |
| 112389 Match Control Crohns-F | 25.0 | 2.3 |
| 112375 Crohns-F | 28.9 | 5.4 |
| 112732 Match Control Crohns-F | 3.1 | 1.1 |
| 112725 Crohns-M | 3.7 | 1.0 |
| 112387 Match Control Crohns-M | 25.0 | 2.3 |
| 112378 Crohns-M | 0.2 | 0.0 |
| 112390 Match Control Crohns-M | 28.3 | 6.6 |
| 112726 Crohns-M | 12.1 | 1.3 |
| 112731 Match Control Crohns-M | 10.7 | 3.5 |
| 112380 Ulcer Col-F | 13.3 | 2.0 |
| 112734 Match Control Ulcer Col-F | 8.3 | 6.2 |
| 112384 Ulcer Col-F | 27.9 | 6.0 |
| 112737 Match Control Ulcer Col-F | 6.4 | 0.0 |
| 112386 Ulcer Col-F | 16.2 | 1.5 |
| 112738 Match Control Ulcer Col-F | 3.6 | 0.0 |
| 112381 Ulcer Col-M | 1.6 | 0.0 |
| 112735 Match Control Ulcer Col-M | 16.4 | 0.0 |
| 112382 Ulcer Col-M | 16.6 | 2.1 |
| 112394 Match Control Ulcer Col-M | 6.8 | 0.0 |
| 112383 Ulcer Col-M | 7.7 | 3.2 |
| 112736 Match Control Ulcer Col-M | 10.5 | 1.3 |
| 112423 Psoriasis-F | 13.2 | 0.0 |
| 112427 Match Control Psoriasis-F | 38.4 | 6.7 |
| 112418 Psoriasis-M | 16.5 | 1.8 |
| 112723 Match Control Psoriasis-M | 13.1 | 0.0 |
| 112419 Psoriasis-M | 31.4 | 3.3 |
| 112424 Match Control Psoriasis-M | 13.0 | 1.4 |
| 112420 Psoriasis-M | 30.6 | 3.2 |
| 112425 Match Control Psoriasis-M | 30.1 | 5.6 |
| 104689 (MF) OA Bone-Backus | 79.6 | 31.6 |
| 104690 (MF) Adj “Normal” Bone-Backus | 53.2 | 5.1 |
| 104691 (MF) OA Synovium-Backus | 43.8 | 10.2 |
| 104692 (BA) OA Cartilage-Backus | 63.3 | 9.4 |
| 104694 (BA) OA Bone-Backus | 64.6 | 20.7 |
| 104695 (BA) Adj “Normal” Bone-Backus | 87.1 | 18.7 |
| 104696 (BA) OA Synovium-Backus | 36.9 | 7.0 |
| 104700 (SS) OA Bone-Backus | 56.3 | 14.4 |
| 104701 (SS) Adj “Normal” Bone-Backus | 100.0 | 25.0 |
| 104702 (SS) OA Synovium-Backus | 62.9 | 13.2 |
| 117093 OA Cartilage Rep7 | 12.6 | 3.2 |
| 112672 OA Bone5 | 27.9 | 5.0 |
| 112673 OA Synovium5 | 8.4 | 2.0 |
| 112674 OA Synovial Fluid cells5 | 8.8 | 0.0 |
| 117100 OA Cartilage Rep14 | 7.2 | 0.0 |
| 112756 OA Bone9 | 51.1 | 100.0 |
| 112757 OA Synovium9 | 4.3 | 1.4 |
| 112758 OA Synovial Fluid Cells9 | 10.8 | 0.0 |
| 117125 RA Cartilage Rep2 | 45.4 | 2.1 |
| 113492 Bone2 RA | 10.8 | 2.9 |
| 113493 Synovium2 RA | 2.7 | 2.0 |
| 113494 Syn Fluid Cells RA | 7.0 | 2.4 |
| 113499 Cartilage4 RA | 7.0 | 4.2 |
| 113500 Bone4 RA | 7.1 | 1.9 |
| 113501 Synovium4 RA | 4.2 | 3.2 |
| 113502 Syn Fluid Cells4 RA | 2.5 | 2.3 |
| 113495 Cartilage3 RA | 5.7 | 4.9 |
| 113496 Bone3 RA | 7.7 | 3.7 |
| 113497 Synovium3 RA | 3.7 | 3.0 |
| 113498 Syn Fluid Cells3 RA | 9.0 | 4.9 |
| 117106 Normal Cartilage Rep20 | 14.4 | 0.0 |
| 113663 Bone3 Normal | 1.3 | 0.0 |
| 113664 Synovium3 Normal | 0.1 | 0.0 |
| 113665 Syn Fluid Cells3 Normal | 1.0 | 0.0 |
| 117107 Normal Cartilage Rep22 | 8.9 | 1.3 |
| 113667 Bone4 Normal | 7.1 | 2.7 |
| 113668 Synovium4 Normal | 7.7 | 2.5 |
| 113669 Syn Fluid Cells4 Normal | 13.8 | 1.3 |

TABLE AE
CNS neurodegeneration v1.0

| | Rel. Exp.(%) |
|---|---|
| | Ag4049, Run |
| Tissue Name | 214292263 |
| AD 1 Hippo | 16.5 |
| AD 2 Hippo | 25.5 |
| AD 3 Hippo | 10.3 |
| AD 4 Hippo | 13.0 |
| AD 5 hippo | 54.3 |
| AD 6 Hippo | 100.0 |
| Control 2 Hippo | 25.9 |
| Control 4 Hippo | 22.7 |
| Control (Path) 3 Hippo | 13.4 |
| AD 1 Temporal Ctx | 15.1 |
| AD 2 Temporal Ctx | 25.3 |
| AD 3 Temporal Ctx | 8.4 |
| AD 4 Temporal Ctx | 18.9 |
| AD 5 Inf Temporal Ctx | 58.6 |
| AD 5 Sup Temporal Ctx | 57.4 |
| AD 6 Inf Temporal Ctx | 82.9 |
| AD 6 Sup Temporal Ctx | 56.6 |
| Control 1 Temporal Ctx | 10.4 |
| Control 2 Temporal Ctx | 16.4 |
| Control 3 Temporal Ctx | 10.8 |
| Control 4 Temporal Ctx | 14.1 |
| Control (Path) 1 Temporal Ctx | 33.0 |
| Control (Path) 2 Temporal Ctx | 23.5 |
| Control (Path) 3 Temporal Ctx | 8.0 |
| Control (Path) 4 Temporal Ctx | 19.9 |
| AD 1 Occipital Ctx | 12.4 |
| AD 2 Occipital Ctx (Missing) | 0.0 |
| AD 3 Occipital Ctx | 9.9 |
| AD 4 Occipital Ctx | 16.2 |
| AD 5 Occipital Ctx | 29.3 |
| AD 6 Occipital Ctx | 27.0 |
| Control 1 Occipital Ctx | 7.6 |
| Control 2 Occipital Ctx | 29.5 |
| Control 3 Occipital Ctx | 17.6 |
| Control 4 Occipital Ctx | 12.7 |
| Control (Path) 1 Occipital Ctx | 33.7 |
| Control (Path) 2 Occipital Ctx | 8.3 |
| Control (Path) 3 Occipital Ctx | 4.5 |
| Control (Path) 4 Occipital Ctx | 11.6 |
| Control 1 Parietal Ctx | 8.4 |
| Control 2 Parietal Ctx | 33.2 |
| Control 3 Parietal Ctx | 13.7 |
| Control (Path) 1 Parietal Ctx | 37.6 |
| Control (Path) 2 Parietal Ctx | 19.5 |
| Control (Path) 3 Parietal Ctx | 8.2 |
| Control (Path) 4 Parietal Ctx | 26.1 |

TABLE AF
General screening panel v1.4

| | Rel. Exp.(%) |
|---|---|
| | Ag4049, Run |
| Tissue Name | 218535058 |
| Adipose | 12.2 |
| Melanoma* Hs688(A).T | 27.0 |
| Melanoma* Hs688(B).T | 29.5 |
| Melanoma* M14 | 21.0 |
| Melanoma* LOXIMVI | 17.4 |
| Melanoma* SK-MEL-5 | 8.8 |
| Squamous cell carcinoma SCC-4 | 0.1 |
| Testis Pool | 10.5 |
| Prostate ca.* (bone met) PC-3 | 10.4 |
| Prostate Pool | 7.9 |
| Placenta | 12.6 |
| Uterus Pool | 5.6 |
| Ovarian ca. OVCAR-3 | 5.8 |
| Ovarian ca. SK-OV-3 | 19.3 |
| Ovarian ca. OVCAR-4 | 4.0 |
| Ovarian ca. OVCAR-5 | 5.4 |
| Ovarian ca. IGROV-1 | 8.2 |
| Ovarian ca. OVCAR-8 | 9.3 |
| Ovary | 10.7 |
| Breast ca. MCF-7 | 1.5 |
| Breast ca. MDA-MB-231 | 29.1 |
| Breast ca. BT 549 | 52.5 |
| Breast ca. T47D | 12.7 |
| Breast ca. MDA-N | 0.0 |
| Breast Pool | 12.1 |
| Trachea | 12.7 |
| Lung | 3.2 |
| Fetal Lung | 38.7 |
| Lung ca. NCI-N417 | 0.1 |
| Lung ca. LX-1 | 10.4 |
| Lung ca. NCI-H146 | 9.1 |
| Lung ca. SHP-77 | 0.2 |
| Lung ca. A549 | 16.8 |
| Lung ca. NCI-H526 | 0.2 |
| Lung ca. NCI-H23 | 6.6 |
| Lung ca. NCI-H460 | 2.3 |
| Lung ca. HOP-62 | 7.9 |
| Lung ca. NCI-H522 | 9.5 |
| Liver | 0.3 |
| Fetal Liver | 2.0 |
| Liver ca. HepG2 | 3.3 |
| Kidney Pool | 23.3 |
| Fetal Kidney | 20.2 |
| Renal ca. 786-0 | 5.9 |
| Renal ca. A498 | 9.1 |
| Renal ca. ACHN | 27.9 |
| Renal ca. UO-31 | 3.4 |
| Renal ca. TK-10 | 9.7 |
| Bladder | 12.0 |
| Gastric ca. (liver met.) NCI-N87 | 0.0 |
| Gastric ca. KATO III | 0.0 |
| Colon ca. SW-948 | 0.0 |
| Colon ca. SW480 | 8.2 |
| Colon ca.* (SW480 met) SW620 | 10.5 |
| Colon ca. HT29 | 0.0 |
| Colon ca. HCT-116 | 13.4 |
| Colon ca. CaCo-2 | 5.7 |
| Colon cancer tissue | 5.6 |
| Colon ca. SW1116 | 0.0 |
| Colon ca. Colo-205 | 0.0 |
| Colon ca. SW-48 | 0.6 |
| Colon Pool | 12.8 |
| Small Intestine Pool | 15.9 |
| Stomach Pool | 6.9 |
| Bone Marrow Pool | 10.9 |
| Fetal Heart | 12.0 |
| Heart Pool | 7.5 |
| Lymph Node Pool | 17.3 |
| Fetal Skeletal Muscle | 19.1 |
| Skeletal Muscle Pool | 6.7 |
| Spleen Pool | 1.9 |
| Thymus Pool | 9.1 |
| CNS cancer (glio/astro) U87-MG | 31.6 |
| CNS cancer (glio/astro) U-118-MG | 63.7 |
| CNS cancer (neuro;met) SK-N-AS | 17.3 |
| CNS cancer (astro) SF-539 | 33.9 |
| CNS cancer (astro) SNB-75 | 51.1 |
| GNS cancer (glio) SNB-19 | 7.6 |
| CNS cancer (glio) SF-295 | 100.0 |
| Brain (Amygdala) Pool | 3.4 |
| Brain (cerebellum) | 82.4 |
| Brain (fetal) | 6.1 |
| Brain (Hippocampus) Pool | 6.5 |
| Cerebral Cortex Pool | 5.4 |
| Brain (Substantia nigra) Pool | 5.0 |
| Brain (Thalamus) Pool | 6.7 |
| Brain (whole) | 6.7 |
| Spinal Cord Pool | 8.7 |
| Adrenal Gland | 5.0 |
| Pituitary gland Pool | 4.1 |
| Salivary Gland | 3.3 |
| Thyroid (female) | 2.0 |
| Pancreatic ca CAPAN2 | 0.8 |
| Pancreas Pool | 15.6 |

TABLE AG
General screening panel v1.5

| | Rel. Exp.(%) |
|---|---|
| | Ag5848, Run |
| Tissue Name | 246273485 |
| Adipose | 1.4 |
| Melanoma* Hs688(A).T | 4.5 |
| Melanoma* Hs688(B).T | 3.6 |
| Melanoma* M14 | 4.1 |
| Melanoma* LOXIMVI | 9.9 |
| Melanoma* SK-MEL-5 | 5.5 |
| Squamous cell carcinoma SCC-4 | 1.4 |
| Testis Pool | 0.6 |
| Prostate ca.* (bone met) PC-3 | 2.5 |
| Prostate Pool | 4.0 |
| Placenta | 2.6 |
| Uterus Pool | 0.1 |
| Ovarian ca. OVCAR-3 | 11.8 |
| Ovarian ca. SK-OV-3 | 11.3 |
| Ovarian ca. OVCAR-4 | 2.2 |
| Ovarian ca. OVCAR-5 | 23.5 |
| Ovarian ca. IGROV-1 | 8.0 |
| Ovarian ca. OVCAR-8 | 3.6 |
| Ovary | 2.0 |
| Breast ca. MCF-7 | 3.6 |
| Breast ca. MDA-MB-231 | 14.0 |
| Breast ca. BT 549 | 4.0 |
| Breast ca. T47D | 8.8 |
| Breast ca. MDA-N | 0.0 |
| Breast Pool | 1.3 |
| Trachea | 1.6 |
| Lung | 0.3 |
| Fetal Lung | 8.2 |
| Lung ca. NCI-N417 | 0.0 |
| Lung ca. LX-1 | 15.4 |
| Lung ca. NCI-H146 | 6.4 |
| Lung ca. SHP-77 | 0.4 |
| Lung ca. A549 | 13.7 |
| Lung ca. NCI-H526 | 0.0 |
| Lung ca. NCI-H23 | 0.9 |
| Lung ca. NCI-H460 | 0.6 |
| Lung ca. HOP-62 | 2.7 |
| Lung ca. NCI-H522 | 26.8 |
| Liver | 0.0 |
| Fetal Liver | 0.3 |
| Liver ca. HepG2 | 6.3 |
| Kidney Pool | 3.8 |
| Fetal Kidney | 3.4 |
| Renal ca. 786-0 | 2.0 |
| Renal ca. A498 | 4.5 |
| Renal ca. ACHN | 12.6 |
| Renal ca. UO-31 | 0.8 |
| Renal ca. TK-10 | 27.9 |
| Bladder | 6.0 |
| Gastric ca. (liver met.) NCI-N87 | 0.0 |
| Gastric ca. KATO III | 0.0 |
| Colon ca. SW-948 | 0.0 |
| Colon ca. SW480 | 71.2 |
| Colon ca.* (SW480 met) SW620 | 11.2 |
| Colon ca. HT29 | 0.0 |
| Colon ca. HCT-116 | 53.6 |
| Colon ca. CaCo-2 | 6.9 |
| Colon cancer tissue | 1.1 |
| Colon ca. SW1116 | 0.0 |
| Colon ca. Colo-205 | 0.0 |
| Colon ca. SW-48 | 0.1 |
| Colon Pool | 1.4 |
| Small Intestine Pool | 0.8 |
| Stomach Pool | 2.0 |
| Bone Marrow Pool | 1.1 |
| Fetal Heart | 0.8 |
| Heart Pool | 0.6 |
| Lymph Node Pool | 2.2 |
| Fetal Skeletal Muscle | 1.5 |
| Skeletal Muscle Pool | 6.7 |
| Spleen Pool | 0.1 |
| Thymus Pool | 4.9 |
| CNS cancer (glio/astro) U87-MG | 18.4 |
| CNS cancer (glio/astro) U-118-MG | 88.9 |
| CNS cancer (neuro;met) SK-N-AS | 15.5 |
| CNS cancer (astro) SF-539 | 5.9 |
| CNS cancer (astro) SNB-75 | 17.4 |
| CNS cancer (glio) SNB-19 | 4.9 |
| CNS cancer (glio) SF-295 | 100.0 |
| Brain (Amygdala) Pool | 0.2 |
| Brain (cerebellum) | 22.2 |
| Brain (fetal) | 1.4 |
| Brain (Hippocampus) Pool | 0.2 |
| Cerebral Cortex Pool | 0.1 |
| Brain (Substantia nigra) Pool | 1.0 |
| Brain (Thalamus) Pool | 0.8 |
| Brain (whole) | 0.8 |
| Spinal Cord Pool | 1.3 |
| Adrenal Gland | 1.4 |
| Pituitary gland Pool | 1.3 |
| Salivary Gland | 2.9 |
| Thyroid (female) | 0.5 |
| Pancreatic ca. CAPAN2 | 0.6 |
| Pancreas Pool | 6.5 |

TABLE AH
Oncology cell line screening panel v3.2

| | Rel. Exp.(%) |
|---|---|
| | Ag4049, Run |
| Tissue Name | 258170122 |
| 94905_Daoy_Medulloblastoma/Cerebellum_sscDNA | 2.2 |
| 94906_TE671_Medulloblastom/Cerebellum_sscDNA | 11.8 |
| 94907_D283 | 2.6 |
| Med_Medulloblastoma/Cerebellum_sscDNA | |
| 94908_PFSK-1_Primitive | 33.0 |
| Neuroectodermal/Cerebellum_sscDNA | |
| 94909_XF-498_CNS_sscDNA | 3.3 |
| 94910_SNB-78_CNS/glioma_sscDNA | 21.2 |
| 94911_SF-268_CNS/glioblastoma_sscDNA | 19.2 |
| 94912_T98G_Glioblastoma_sscDNA | 9.5 |
| 96776_SK-N-SH_Neuroblastoma | 5.6 |
| (metastasis)_sscDNA | |
| 94913_SF-295_CNS/glioblastoma_sscDNA | 13.4 |
| 132565_NT2 pool_sscDNA | 45.1 |
| 94914_Cerebellum_sscDNA | 25.3 |
| 96777_Cerebellum_sscDNA | 27.9 |
| 94916_NCI-H292_Mucoepidermoid lung | 0.4 |
| carcinoma_sscDNA | |
| 94917_DMS-114_Small cell lung cancer_sscDNA | 16.8 |
| 94918_DMS-79_Small cell lung | 1.4 |
| cancer/neuroendocrine_sscDNA | |
| 94919_NCI-H146_Small cell lung | 22.8 |
| cancer/neuroendocrine_sscDNA | |
| 94920_NCI-H526_Small cell lung | 0.9 |
| cancer/neuroendocrine_sscDNA | |
| 94921_NCI-N417_Small cell lung | 0.5 |
| cancer/neuroendocrine_sscDNA | |
| 94923_NCI-H82_Small cell lung | 6.0 |
| cancer/neuroendocrine_sscDNA | |
| 94924_NCI-H157_Squamous cell lung | 4.4 |
| cancer (metastasis)_sscDNA | |
| 94925_NCI-H1155_Large cell lung | 3.2 |
| cancer/neuroendocrine_sscDNA | |
| 94926_NCI-H1299_Large cell lung | 13.5 |
| cancer/neuroendocrine_sscDNA | |
| 94927_NCI-H727_Lung carcinoid_sscDNA | 4.9 |
| 94928_NCI-UMC-11_Lung carcinoid_sscDNA | 22.1 |
| 94929_LX-1_Small cell lung cancer_sscDNA | 6.3 |
| 94930_Colo-205_Colon cancer_sscDNA | 0.0 |
| 94931_KM12_Colon cancer_sscDNA | 0.0 |
| 94932_KM20L2_Colon cancer_sscDNA | 0.0 |
| 94933_NCI-H716_Colon cancer_sscDNA | 0.1 |
| 94935_SW-48_Colon adenocarcinoma_sscDNA | 1.3 |
| 94936_SW1116_Colon adenocarcinoma_sscDNA | 0.0 |
| 94937_LS 174T_Colon adenocarcinoma_sscDNA | 0.5 |
| 94938_SW-948_Colon adenocarcinoma_sscDNA | 0.0 |
| 94939_SW-480_Colon adenocarcinoma_sscDNA | 0.0 |
| 94940_NCI-SNU-5_Gastric carcinoma_sscDNA | 7.6 |
| 112197_KATO III_Stomach_sscDNA | 0.0 |
| 94943_NCI-SNU-16_Gastric carcinoma_sscDNA | 11.0 |
| 94944_NCI-SNU-1_Gastric carcinoma_sscDNA | 0.0 |
| 94946_RF-1_Gastric adenocarcinoma_sscDNA | 1.8 |
| 94947_RF-48_Gastric adenocarcinoma_sscDNA | 2.1 |
| 96778_MKN-45_Gastric carcinoma_sscDNA | 4.4 |
| 94949_NCI-N87_Gastric carcinoma_sscDNA | 0.0 |
| 94951_OVCAR-5_Ovarian carcinoma_sscDNA | 3.4 |
| 94952_RL95-2_Uterine carcinoma_sscDNA | 0.0 |
| 94953_HelaS3_Cervical adenocarcinoma_sscDNA | 5.0 |
| 94954_Ca Ski_Cervical epidermoid | 3.1 |
| carcinoma (metastasis)_sscDNA | |
| 94955_ES-2_Ovarian clear cell carcinoma_sscDNA | 9.2 |
| 94957_Ramos/6h stim_Stimulated | 0.0 |
| with PMA/ionomycin 6h_sscDNA | |
| 94958_Ramos/14h stim_Stimulated | 0.2 |
| with PMA/ionomycin 14h_sscDNA | |
| 94962_MEG-01_Chronic myelogenous leukemia | 6.3 |
| (megokaryoblast)_sscDNA | |
| 94963_Raji_Burkitt's lymphoma_sscDNA | 0.0 |
| 94964_Daudi_Burkitt's lymphoma_sscDNA | 0.6 |
| 94965_U266_B-cell plasmacytoma/myeloma_sscDNA | 0.3 |
| 94968_CA46_Burkitt's lymphoma_sscDNA | 0.0 |
| 94970_RL_non-Hodgkin's B-cell lymphoma_sscDNA | 0.0 |
| 94972_JM1_pre-B-cell lymphoma/leukemia_sscDNA | 0.2 |
| 94973_Jurkat_T cell leukemia_sscDNA | 3.7 |
| 94974_TF-1_Erythroleukemia_sscDNA | 1.8 |
| 94975_HUT 78_T-cell lymphoma_sscDNA | 11.7 |
| 94977_U937_Histiocytic lymphoma_sscDNA | 0.4 |
| 94980_KU-812_Myelogenous leukemia_sscDNA | 0.0 |
| 94981_769-P_Clear cell renal carcinoma_sscDNA | 18.7 |
| 94983_Caki-2_Clear cell renal carcinoma_sscDNA | 6.0 |
| 94984_SW 839_Clear cell renal carcinoma_sscDNA | 34.4 |
| 94986_G401_Wilms′ tumor_sscDNA | 11.4 |
| 126768_293 cells_sscDNA | 2.9 |
| 94987_Hs766T_Pancreatic | 13.0 |
| carcinoma (LN metastasis)_sscDNA | |
| 94988_CAPAN-1_Pancreatic adenocarcinoma | 0.0 |
| (liver metastasis)_sscDNA | |
| 94989_SU86.86_Pancreatic carcinoma (liver | 1.4 |
| metastasis)_sscDNA | |
| 94990_BxPC-3_Pancreatic adenocarcinoma_sscDNA | 0.0 |
| 94991_HPAC_Pancreatic adenocarcinoma_sscDNA | 0.4 |
| 94992_MIA PaCa-2_Pancreatic carcinoma_sscDNA | 0.8 |
| 94993_CFPAC-1_Pancreatic ductal | 4.5 |
| adenocarcinoma_sscDNA | |
| 94994_PANC-1_Pancreatic epithelioid ductal | 7.0 |
| carcinoma_sscDNA | |
| 94996_T24_Bladder carcinma | 1.8 |
| (transitional cell)_sscDNA | |
| 94997_5637_Bladder carcinoma_sscDNA | 1.7 |
| 94998_HT-1197_Bladder carcinoma_sscDNA | 4.6 |
| 94999_UM-UC-3_Bladder carcinma | 6.1 |
| (transitional cell)_sscDNA | |
| 95000_A204_Rhabdomyosarcoma_sscDNA | 100.0 |
| 95001_HT-1080_Fibrosarcoma_sscDNA | 9.2 |
| 95002_MG-63_Osteosarcoma (bone)_sscDNA | 22.7 |
| 95003_SK-LMS-1_Leiomyosarcom a (vulva)_sscDNA | 23.5 |
| 95004_SJRH30_Rhabdomyosarcom | 2.7 |
| a (met to bone marrow)_sscDNA | |
| 95005_A431_Epidermoid carcinoma_sscDNA | 0.0 |
| 95007_WM266-4_Melanoma_sscDNA | 4.0 |
| 112195_DU 145_Prostate_sscDNA | 11.3 |
| 95012_MDA-MB-468_Breast | 1.5 |
| adenocarcinoma_sscDNA | |
| 112196_SSC-4_Tongue_sscDNA | 0.4 |
| 112194_SSC-9_Tongue_sscDNA | 0.1 |
| 112191_SSC-15_Tongue_sscDNA | 0.0 |
| 95017_CAL 27_Squamous cell | 0.0 |
| carcinoma of tongue_sscDNA | |

TABLE AI
Panel 4.1D

| | Rel. | Rel. |
|---|---|---|
| | Exp. (%) | Exp. (%) |
| | Ag4049, | Ag5848, |
| | Run | Run |
| Tissue Name | 171619851 | 246921472 |
| Secondary Th1 act | 8.2 | 39.8 |
| Secondary Th2 act | 4.7 | 19.5 |
| Secondary Tr1 act | 4.0 | 2.8 |
| Secondary Th1 rest | 2.0 | 0.0 |
| Secondary Th2 rest | 3.4 | 7.1 |
| Secondary Tr1 rest | 5.9 | 0.0 |
| Primary Th1 act | 5.8 | 0.0 |
| Primary Th2 act | 3.5 | 19.9 |
| Primary Tr1 act | 4.7 | 14.2 |
| Primary Th1 rest | 5.3 | 1.5 |
| Primary Th2 rest | 3.9 | 8.1 |
| Primary Tr1 rest | 2.0 | 0.0 |
| CD45RA CD4 lymphocyte act | 28.1 | 31.0 |
| CD45RO CD4 lymphocyte act | 3.5 | 6.6 |
| CD8 lymphocyte act | 5.4 | 3.1 |
| Secondary CD8 lymphocyte rest | 0.5 | 0.0 |
| Secondary CD8 lymphocyte act | 6.7 | 6.0 |
| CD4 lymphocyte none | 0.8 | 0.0 |
| 2ry Th1/Th2/Tr1_anti-CD95 CH11 | 6.3 | 0.0 |
| LAK cells rest | 2.7 | 3.5 |
| LAK cells IL-2 | 2.5 | 6.8 |
| LAK cells IL-2 + IL-12 | 2.1 | 0.0 |
| LAK cells IL-2 + IFN gamma | 1.9 | 0.0 |
| LAK cells IL-2 + IL-18 | 3.2 | 0.0 |
| LAK cells PMA/ionomycin | 10.6 | 46.0 |
| NK Cells IL-2 rest | 4.7 | 18.0 |
| Two Way MLR 3 day | 0.9 | 0.0 |
| Two Way MLR 5 day | 4.5 | 3.7 |
| Two Way MLR 7 day | 15.1 | 0.0 |
| PBMC rest | 0.8 | 2.0 |
| PBMC PWM | 1.7 | 3.9 |
| PBMC PHA-L | 6.6 | 6.4 |
| Ramos (B cell) none | 0.0 | 0.0 |
| Ramos (B cell) ionomycin | 0.0 | 0.0 |
| B lymphocytes PWM | 2.9 | 6.4 |
| B lymphocytes CD40L and IL-4 | 2.0 | 9.9 |
| EOL-1 dbcAMP | 1.8 | 2.3 |
| EOL-1 dbcAMP PMA/ionomycin | 1.6 | 0.0 |
| Dendritic cells none | 2.5 | 1.6 |
| Dendritic cells LPS | 1.9 | 0.0 |
| Dendritic cells anti-CD40 | 6.5 | 0.0 |
| Monocytes rest | 0.2 | 0.0 |
| Monocytes LPS | 0.8 | 4.3 |
| Macrophages rest | 12.6 | 5.8 |
| Macrophages LPS | 1.9 | 4.4 |
| HUVEC none | 13.3 | 7.0 |
| HUVEC starved | 21.6 | 29.1 |
| HUVEC IL-1beta | 13.2 | 8.1 |
| HUVEC IFN gamma | 16.4 | 39.0 |
| HUVEC TNF alpha + IFN gamma | 8.2 | 0.0 |
| HUVEC TNF alpha + IL4 | 8.1 | 0.0 |
| HUVEC IL-11 | 12.9 | 7.9 |
| Lung Microvascular EC none | 14.7 | 22.5 |
| Lung Microvascular EC TNFalpha + | 5.4 | 3.1 |
| IL-1beta | | |
| Microvascular Dermal EC none | 12.3 | 0.0 |
| Microsvasular Dermal EC TNFalpha + | 4.3 | 6.7 |
| IL-1beta | | |
| Bronchial epithelium TNFalpha + | 0.9 | 29.1 |
| IL1beta | | |
| Small airway epithelium none | 1.3 | 13.6 |
| Small airway epithelium TNFalpha + | 0.3 | 10.3 |
| IL-1beta | | |
| Coronery artery SMC rest | 9.7 | 10.8 |
| Coronery artery SMC TNFalpha + | 11.0 | 4.3 |
| IL-1beta | | |
| Astrocytes rest | 26.4 | 19.1 |
| Astrocytes TNFalpha + IL-1beta | 28.5 | 25.9 |
| KU-812 (Basophil) rest | 0.0 | 2.6 |
| KU-812 (Basophil) PMA/ionomycin | 0.3 | 1.7 |
| CCD1106 (Keratinocytes) none | 1.1 | 12.1 |
| CCD1106 (Keratinocytes) TNFalpha + | 0.2 | 6.7 |
| IL-1beta | | |
| Liver cirrhosis | 7.2 | 0.0 |
| NCI-H292 none | 0.1 | 0.0 |
| NCI-H292 IL-4 | 0.3 | 0.0 |
| NCI-H292 IL-9 | 0.4 | 0.0 |
| NCI-H292 IL-13 | 1.1 | 0.0 |
| NCI-H292 IFN gamma | 0.4 | 2.1 |
| HPAEC none | 18.0 | 5.2 |
| HPAEC TNF alpha + IL-1 beta | 9.9 | 25.5 |
| Lung fibroblast none | 64.2 | 48.6 |
| Lung fibroblast TNF alpha + IL-1 | 62.9 | 69.3 |
| beta | | |
| Lung fibroblast IL-4 | 52.5 | 16.8 |
| Lung fibroblast IL-9 | 100.0 | 39.2 |
| Lung fibroblast IL-13 | 57.8 | 5.9 |
| Lung fibroblast IFN gamma | 76.3 | 51.8 |
| Dermal fibroblast CCD1070 rest | 42.3 | 51.4 |
| Dermal fibroblast CCD1070 TNF alpha | 49.3 | 94.6 |
| Dermal fibroblast CCD1070 IL-1 beta | 34.6 | 27.4 |
| Dermal fibroblast IFN gamma | 46.3 | 47.6 |
| Dermal fibroblast IL-4 | 63.3 | 100.0 |
| Dermal Fibroblasts rest | 43.2 | 30.4 |
| Neutrophils TNFa + LPS | 3.3 | 0.0 |
| Neutrophils rest | 6.0 | 0.0 |
| Colon | 6.3 | 0.0 |
| Lung | 9.5 | 0.0 |
| Thymus | 12.0 | 1.9 |
| Kidney | 8.7 | 12.1 |

TABLE AJ
Panel 5D

| | Rel. | Rel. |
|---|---|---|
| | Exp. (%) | Exp. (%) |
| | Ag449, | Ag5848, |
| | Run | Run |
| Tissue Name | 257488285 | 257488388 |
| 97457_Patient-02go_adipose | 29.9 | 17.1 |
| 97476_Patient-07sk_skeletal muscle | 17.3 | 19.5 |
| 97477_Patient-07ut_uterus | 33.7 | 5.2 |
| 97478_Patient-07pl_placenta | 18.8 | 0.0 |
| 97481_Patient-08sk_skeletal muscle | 18.0 | 15.8 |
| 97482_Patient-08ut_uterus | 22.5 | 0.0 |
| 97483_Patient-08pl_placenta | 10.2 | 0.0 |
| 97486_Patient-09sk_skeletal muscle | 4.1 | 4.7 |
| 97487_Patient-09ut_uterus | 36.3 | 8.7 |
| 97488_Patient-09pl_placenta | 12.4 | 13.5 |
| 97492_Patient-10ut_uterus | 42.9 | 19.9 |
| 97493_Patient-10pl_placenta | 16.8 | 17.0 |
| 97495_Patient-11go_adipose | 22.5 | 16.5 |
| 97496_Patient-11sk_skeletal muscle | 11.6 | 0.0 |
| 97497_Patient-11ut_uterus | 67.4 | 8.8 |
| 97498_Patient-11pl_placenta | 13.3 | 9.5 |
| 97500_Patient-12go_adipose | 33.0 | 16.5 |
| 97501_Patient-12sk_skeletal muscle | 29.3 | 12.3 |
| 97502_Patient-12ut_uterus | 59.0 | 18.8 |
| 97503_Patient-12pl_placenta | 7.8 | 0.0 |
| 94721_Donor 2 U - A_Mesenchymal Stem | 39.0 | 12.3 |
| Cells | | |
| 94722_Donor 2 U - B_Mesenchymal Stem | 33.7 | 21.6 |
| Cells | | |
| 94723_Donor 2 U - C_Mesenchymal Stem | 33.2 | 19.9 |
| Cells | | |
| 94709_Donor 2 AM - A_adipose | 53.2 | 30.8 |
| 94710_Donor 2 AM - B_adipose | 33.4 | 3.2 |
| 94711_Donor 2 AM - C_adipose | 26.8 | 21.2 |
| 94712_Donor 2 AD - A_adipose | 66.4 | 88.9 |
| 94713_Donor 2 AD - B_adipose | 100.0 | 87.7 |
| 94714_Donor 2 AD - C_adipose | 88.9 | 50.3 |
| 94742_Donor 3 U - A_Mesenchymal Stem | 26.2 | 19.6 |
| Cells | | |
| 94743_Donor 3 U - B_Mesenchymal Stem | 24.1 | 19.8 |
| Cells | | |
| 94730_Donor 3 AM - A_adipose | 62.9 | 35.4 |
| 94731_Donor 3 AM - B_adipose | 32.5 | 25.7 |
| 94732_Donor 3 AM - C_adipose | 33.7 | 41.2 |
| 94733_Donor 3 AD - A_adipose | 77.9 | 49.7 |
| 94734_Donor 3 AD - B_adipose | 49.0 | 45.1 |
| 94735_Donor 3 AD - C_adipose | 58.2 | 64.6 |
| 77138_Liver_HepG2 untreated | 12.2 | 16.2 |
| 73556_Heart_Cardiac stromal cells | 14.8 | 0.0 |
| (primary) | | |
| 81735_Small Intestine | 29.3 | 6.2 |
| 72409_Kidney_Proximal Convoluted | 11.4 | 23.0 |
| Tubule | | |
| 82685_Small intestine_Duodenum | 2.2 | 0.0 |
| 90650_Adrenal_Adrenocortical adenoma | 1.7 | 0.0 |
| 72410_Kidney_HRCE | 75.8 | 100.0 |
| 72411_Kidney_HRE | 27.4 | 31.2 |
| 73139_Uterus_Uterine smooth muscle | 17.3 | 0.0 |
| cells | | |

TABLE AK
general oncology screening panel v 2.4

| | Rel. Exp.(%) |
|---|---|
| | Ag4049, Run |
| Tissue Name | 268362940 |
| Colon cancer 1 | 6.7 |
| Colon NAT 1 | 11.1 |
| Colon cancer 2 | 6.7 |
| Colon NAT 2 | 3.5 |
| Colon cancer 3 | 8.5 |
| Colon NAT 3 | 14.8 |
| Colon malignant cancer 4 | 10.5 |
| Colon NAT 4 | 3.4 |
| Lung cancer 1 | 9.0 |
| Lung NAT 1 | 1.7 |
| Lung cancer 2 | 47.3 |
| Lung NAT 2 | 3.0 |
| Squamous cell carcinoma 3 | 16.6 |
| Lung NAT 3 | 1.3 |
| Metastatic melanoma 1 | 23.7 |
| Melanoma 2 | 7.5 |
| Melanoma 3 | 4.4 |
| Metastatic melanoma 4 | 59.9 |
| Metastatic melanoma 5 | 80.7 |
| Bladder cancer 1 | 2.4 |
| Bladder NAT 1 | 0.0 |
| Bladder cancer 2 | 3.0 |
| Bladder NAT 2 | 1.6 |
| Bladder NAT 3 | 0.7 |
| Bladder NAT 4 | 12.9 |
| Prostate adenocarcinoma 1 | 43.5 |
| Prostate adenocarcinoma 2 | 3.4 |
| Prostate adenocarcinoma 3 | 6.0 |
| Prostate adenocarcinoma 4 | 6.7 |
| Prostate NAT 5 | 7.1 |
| Prostate adenocarcinoma 6 | 1.7 |
| Prostate adenocarcinoma 7 | 5.7 |
| Prostate adenocarcinoma 8 | 1.5 |
| Prostate adenocarcinoma 9 | 25.5 |
| Prostate NAT 10 | 2.6 |
| Kidney cancer 1 | 12.9 |
| Kidney NAT 1 | 9.4 |
| Kidney cancer 2 | 100.0 |
| Kidney NAT 2 | 10.3 |
| Kidney cancer 3 | 12.8 |
| Kidney NAT 3 | 3.7 |
| Kidney cancer 4 | 14.2 |
| Kidney NAT 4 | 2.9 |

AI.05 chondrosarcoma Summary: Ag5848 Highest expression of this gene is detected in untreated serum starved chondrosarcoma cell line (SW1353) (CT=31.9). Interestingly, expression of this gene appears to be somewhat down regulated upon PMA treatment for 18 hrs. Moderate to low levels of expression of this gene is seen in untreated and IL-1 treated chondrosarcoma cells. Modulation of the expression of this transcript in chondrocytes by either small molecules or antisense might be important for preventing the degeneration of cartilage observed in OA.

In addition, repair of osteoarthritis and rheumatoid arthritis tissue is envisioned by the application of FGF's that activate this receptor splice variant. Furthermore, small molecule ligands or agonist therapeutic antibodies may also result in beneficial effects in patients expressing this FGF-receptor splice variant on cells in arthritic lesions.

AI_Comprehensive panel_v1.0 Summary: Ag4049 This gene shows a ubiquitous expression with highest expression in normal bone. Moderate to high expression of this gene are detected in samples derived from normal and orthoarthitis/rheumatoid arthritis bone and adjacent bone, cartilage, synovium and synovial fluid samples, from normal lung, COPD lung, emphysema, atopic asthma, asthma, allergy, Crohn's disease (normal matched control and diseased), ulcerative colitis (normal matched control and diseased), and psoriasis (normal matched control and diseased). Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis.

Ag5848 Low expression of this gene is seen exclusively in OA bone (CT=33). Therefore, expression of this gene may be used as diagnostic marker to detect OA bone and furthermore, therapeutic modulation of this gene may be useful in the treatment of orthoarthritis.

CNS_neurodegeneration_v1.0 Summary: Ag4049 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of this gene in treatment of central nervous system disorders.

General_screening_panel_v1.4 Summary: Ag4049 Highest expression of this gene is detected in CNS cancer (glio) SF-295 cell line (CT=23). High to moderate expression of this gene is also seen in cluster of cancer cell lines derived from pancreatic, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate to high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag5848 Highest expression of this gene is detected in CNS cancer (glio) SF-295 cell line (CT=28.9). Moderate to low expression of this gene is also seen in number of cancer cell lines derived from colon, lung, liver, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, melanoma and brain cancers.

In addition, moderate to low expression of this gene is also seen in pancreas, salivary gland, cerebellum, bladder, kidney, thymus, skeletal muscle, fetal lung, prostate and placenta. Therefore, therapeutic modulation of this gene may be useful in the treatment of diseases related to these tissues.

Interestingly, this gene is expressed at much higher levels in fetal (CT=32.5) when compared to adult lung (CT=37). This observation suggests that expression of this gene can be used to distinguish fetal from adult lung. In addition, the relative overexpression of this gene in fetal tissue suggests that the protein product may enhance lung growth or development in the fetus and thus may also act in a regenerative capacity in the adult. Therefore, therapeutic modulation of the protein encoded by this gene could be useful in treatment of lung related diseases.

Oncology cell_line_screening_panel_v3.2 Summary: Ag4049 Highest expression of this gene is detected in rhabdomyosarcoma sample (CT=26.1). Significant expression of this gene is detected in cerebellum and number of cancer cell lines derived from prostate, melanoma, bone, vulva, bladder, pancreatic, renal, T cell lymphoma and leukemia, erythroleukemia, cervical, ovarian, gastric, colon, lung and brain cancers. Therefore, therapeutic modulation of this gene may be useful in the treatment of these cancers.

Panel 4.1D Summary: Ag4049 Highest expression of this gene is detected in IL-9 treated lung fibroblasts (CT=25.4). This gene shows ubiquitous expression with higher expression in resting and activated lung and dermal fibroblasts. Moderate to low expression of this gene is seen in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

In another experiment using probe-primer set Ag5848, this gene shows low expression in resting and activated lung and dermal fibroblasts (CTs=33.5-34.5).

Panel 5D Summary: Ag4049 This gene shows ubiquitous expression with highest expression seen in differentiated adipose (CT=27.5). This gene is not differentially expressed when comparing tissue (adipose and skeletal muscle) from gestationally diabetic women with varying BMI. Please see panel 1.4 for further discussion of this gene.

In another experiment using probe-primer set Ag5848, this gene shows low expression in kidney and differentiated adipose tissue (CTs=33.9-34).

general oncology screening panel_v_{—}2.4 Summary: Ag4049 Highest expression of this gene is detected in kidney cancer (CT=25.2). High to moderate expression of this gene is seen in normal adjacent and cancer samples derived from kidney, prostate, bladder, melanoma, lung and colon. Expression of this gene is higher in metastic melanoma, lung and kidney cancer compared to corresponding normal tissue. Therefore, expression of this gene may be used as diagnostic marker to detect the presence of metastatic melanoma, kidney and lung cancer. Furthermore, therapeutic modulation of this gene or its protein product may be useful in the treatment of melanoma, kidney, prostate, bladder, lung and colon cancers.

B. CG102006-02: Human Peroxiredoxin 2-Like Protein.

Expression of gene CG102006-02 was assessed using the primer-probe set Ag6536, described in Table BA.

TABLE BA

Probe Name Ag6536

| | | | Start | SEQ ID |
|---|---|---|---|---|
| Primers | Sequencs | Length | Position | No |
| Forward | 5′-gttgcctggtatatagagttgca-3′ | 23 | 513 | 488 |
| Probe | TET-5′-tgcaactcagatgcaactctatctactc-3′-TAMRA | 28 | 538 | 489 |
| Reverse | 5′-ccctcctgggaactaagtaca-3′ | 21 | 568 | 490 |

C. CG127322-01, CG127322-02, CG127322-03 and CG127322-04:Human Kynurenine Hydroxylase-Like Protein.

Expression of gene CG127322-01, CG127322-02, CG127322-03 and CG127322-04 was assessed using the primer-probe sets Ag4744, Ag6981 and Ag6998, described in Tables CA, CB and CC. Results of the RTQ-PCR runs are shown in Tables CD and CE. Please note that CG127322-02 represents a full-length physical clone of the CG127322-01 gene, validating the prediction of the gene sequence. In addition, CG127322-03 and CG127322-04 also represents a full-length physical clones. Also, Ag6998 is specific for CG127322-03 and CG127322-04, while Ag6981 is specific for CG127322-04.

TABLE CA

Probe Name Ag4744

| | | | Start | SEQ ID |
|---|---|---|---|---|
| Primers | Sequencs | Length | Position | No |
| Forward | 5′-cagtgcttggatctgacaaagt-3′ | 22 | 452 | 491 |
| Probe | TET-5′-tcccaaagatgtcacttgtgacctca-3′-TAMRA | 26 | 474 | 492 |
| Reverse | 5′-gacagttgaataggctccatca-3′ | 22 | 510 | 493 |

TABLE CB

Probe Name Ag6981

| | | | Start | SEQ ID |
|---|---|---|---|---|
| Primers | Sequencs | Length | Position | No |
| Forward | 5′-ctttgattacagtcagcagtacattc-3′ | 26 | 558 | 494 |
| Probe | TET-5′-tggaatagtcaactccatgtacccatga-3′-TAMRA | 28 | 585 | 495 |
| Reverse | 5′-gaatgatttgttatctccgttcttag-3′ | 26 | 614 | 496 |

TABLE CC

Probe Name Ag6998

| | | | Start | SEQ ID |
|---|---|---|---|---|
| Primers | Sequence | Length | Position | No |
| Forward | 5′-tacatagagaagaacatggagaga-3′ | 24 | 1030 | 497 |
| Probe | TET-5′-tgcgattatgccatcgacctttatccc-3′-TAMRA | 27 | 1062 | 498 |
| Reverse | 5′-cctcatggtatcttattctgga-3 | 22 | 1111 | 499 |

TABLE CD
General_screening_panel_v1.4

| | Rel. | | Rel. |
|---|---|---|---|
| | Exp. (%) | | Exp. (%) |
| | Ag4744, | | Ag4744, |
| | Run | | Run |
| Tissue Name | 213829150 | Tissue Name | 213829150 |
| Adipose | 2.0 | Renal ca. TK-10 | 0.2 |
| Melanoma* Hs688(A).T | 0.0 | Bladder | 2.1 |
| Melanoma* Hs688(B).T | 0.0 | Gastric ca. (liver met.) NCI-N87 | 0.4 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 1.0 | Colon ca. SW480 | 0.0 |
| Squamous cell carcinoma SCC-4 | 1.1 | Colon ca.* (SW480 met) SW620 | 0.0 |
| Testis Pool | 0.8 | Colon ca. HT29 | 0.0 |
| Prostate ca.* (bone met) PC-3 | 1.0 | Colon ca. HCT-116 | 0.0 |
| Prostate Pool | 0.3 | Colon ca. CaCo-2 | 0.1 |
| Placenta | 6.3 | Colon cancer tissue | 3.0 |
| Uterus Pool | 0.0 | Colon ca. SW1116 | 0.0 |
| Ovarian ca. OVCAR-3 | 2.1 | Colon ca. Colo-205 | 0.0 |
| Ovarian ca. SK-OV-3 | 0.2 | Colon ca. SW-48 | 0.0 |
| Ovarian ca. OVCAR-4 | 0.0 | Colon Pool | 4.4 |
| Ovarian ca. OVCAR-5 | 2.8 | Small Intestine Pool | 0.4 |
| Ovarian ca. IGROV-1 | 0.9 | Stomach Pool | 4.9 |
| Ovarian ca. OVCAR-8 | 0.0 | Bone Marrow Pool | 0.4 |
| Ovary | 0.5 | Fetal Heart | 0.1 |
| Breast ca. MCF-7 | 1.2 | Heart Pool | 0.0 |
| Breast ca. MDA-MB-231 | 0.0 | Lymph Node Pool | 13.6 |
| Breast ca. BT 549 | 52.9 | Fetal Skeletal Muscle | 0.0 |
| Breast ca. T47D | 6.1 | Skeletal Muscle Pool | 0.3 |
| Breast ca. MDA-N | 0.1 | Spleen Pool | 7.1 |
| Breast Pool | 21.2 | Thymus Pool | 21.5 |
| Trachea | 0.6 | CNS cancer (glio/astro) U87-MG | 0.0 |
| Lung | 0.0 | CNS cancer (glio/astro) U-118-MG | 0.2 |
| Fetal Lung | 1.8 | CNS cancer (neuro; met) SK-N-AS | 0.0 |
| Lung ca. NCI-N417 | 0.0 | CNS cancer (astro) SF-539 | 0.1 |
| Lung ca. LX-1 | 0.0 | CNS cancer (astro) SNB-75 | 0.0 |
| Lung ca. NCI-H146 | 0.2 | CNS cancer (glio) SNB-19 | 0.1 |
| Lung ca. SHP-77 | 0.0 | CNS cancer (glio) SF-295 | 0.9 |
| Lung ca. A549 | 0.7 | Brain (Amygdala) Pool | 0.3 |
| Lung ca. NCI-H526 | 0.0 | Brain (cerebellum) | 0.0 |
| Lung ca. NCI-H23 | 0.1 | Brain (fetal) | 0.4 |
| Lung ca. NCI-H460 | 0.6 | Brain (Hippocampus) Pool | 0.4 |
| Lung ca. HOP-62 | 0.0 | Cerebral Cortex Pool | 0.2 |
| Lung ca. NCI-H522 | 0.0 | Brain (Substantia nigra) Pool | 0.0 |
| Liver | 9.3 | Brain (Thalamus) Pool | 0.0 |
| Fetal Liver | 47.3 | Brain (whole) | 0.5 |
| Liver ca. HepG2 | 0.0 | Spinal Cord Pool | 0.5 |
| Kidney Pool | 0.5 | Adrenal Gland | 0.3 |
| Fetal Kidney | 9.9 | Pituitary gland Pool | 0.0 |
| Renal ca. 786-0 | 100.0 | Salivary Gland | 0.2 |
| Renal ca. A498 | 11.0 | Thyroid (female) | 0.7 |
| Renal ca. ACHN | 1.4 | Pancreatic ca. CAPAN2 | 0.0 |
| Renal ca UO-31 | 1.5 | Pancreas Pool | 18.7 |

TABLE CE
Panel 5 Islet

| | Rel. | Rel. |
|---|---|---|
| | Exp. (%) | Exp. (%) |
| | Ag4744 | Ag6998, |
| | Run | Run |
| Tissue Name | 204244613 | 284710396 |
| 97457_Patient-02go_adipose | 3.5 | 0.6 |
| 97476_Patient-07sk_skeletal muscle | 0.0 | 0.0 |
| 97477_Patient-07ut_uterus | 0.0 | 0.0 |
| 97478_Patient-07pl_placenta | 43.2 | 43.5 |
| 99167_Bayer Patient 1 | 6.2 | 0.0 |
| 97482_Patient-08ut_uterus | 1.0 | 0.6 |
| 97483_Patient-08pl_placenta | 60.7 | 40.6 |
| 97486_Patient-09sk_skeletal muscle | 0.0 | 0.0 |
| 97487_Patient-09ut_uterus | 1.9 | 0.0 |
| 97488_Patient-09pl_placenta | 47.3 | 27.4 |
| 97492_Patient-10ut_uterus | 0.0 | 0.0 |
| 97493_Patient-10pl_placenta | 71.7 | 66.0 |
| 97495_Patient-11go_adipose | 2.5 | 0.0 |
| 97496_Patient-11sk_skeletal muscle | 0.0 | 0.0 |
| 97497_Patient-11ut_uterus | 1.8 | 0.7 |
| 97498_Patient-11pl_placenta | 17.9 | 9.7 |
| 97500_Patient-12go_adipose | 4.4 | 1.2 |
| 97501_Patient-12sk_skeletal muscle | 0.0 | 0.0 |
| 97502_Patient-12ut_uterus | 1.2 | 1.7 |
| 97503_Patient-12pl_placenta | 100.0 | 100.0 |
| 94721_Donor 2 U - A_Mesenchymal Stem | 0.0 | 0.0 |
| Cells | | |
| 94722_Donor 2 U - B_Mesenchymal Stem | 0.0 | 0.0 |
| Cells | | |
| 94723_Donor 2 U - C_Mesenchymal Stem | 0.0 | 0.0 |
| Cells | | |
| 94709_Donor 2 AM - A_adipose | 0.0 | 0.0 |
| 94710_Donor 2 AM - B_adipose | 0.0 | 0.0 |
| 94711_Donor 2 AM - C_adipose | 0.0 | 0.7 |
| 94712_Donor 2 AD - A_adipose | 0.0 | 0.0 |
| 94713_Donor 2 AD - B_adipose | 0.0 | 0.0 |
| 94714_Donor 2 AD - C_adipose | 0.0 | 0.0 |
| 94742_Donor 3 U - A_Mesenchymal Stem | 0.0 | 0.0 |
| Cells | | |
| 94743_Donor 3 U - B_Mesenchymal Stem | 0.0 | 0.0 |
| Cells | | |
| 94730_Donor 3 AM - A_adipose | 0.0 | 0.0 |
| 94731_Donor 3 AM - B_adipose | 0.0 | 0.0 |
| 94732_Donor 3 AM - C_adipose | 0.0 | 0.0 |
| 94733_Donor 3 AD - A_adipose | 0.0 | 0.0 |
| 94734_Donor 3 AD - B_adipose | 0.0 | 0.0 |
| 94735_Donor 3 AD - C_adipose | 0.0 | 0.0 |
| 77138_Liver_HepG2 untreated | 0.0 | 0.0 |
| 73556_Heart_Cardiac stromal cells | 0.0 | 0.0 |
| (primary) | | |
| 81735_Small Intestine | 2.5 | 1.3 |
| 72409_Kidney_Proximal Convoluted | 1.2 | 4.0 |
| Tubule | | |
| 82685_Small intestine Duodenum | 0.0 | 1.1 |
| 90650_Adrenal_Adrenocortical adenoma | 4.6 | 1.2 |
| 72410_Kidney_HRCE | 2.3 | 1.4 |
| 72411_Kidney_HRE | 0.0 | 0.6 |
| 73139_Uterus_Uterine smooth muscle | 0.0 | 0.0 |
| cells | | |

General_screening_panel_v1.4 Summary: Ag4744 Highest expression of this gene is detected in a renal cancer 786-0 cell line (CT=30.5). Moderate to low expression of this gene is also seen in renal cancer A498 cell line, breast cancer BT 549 and T47D cell lines. Therefore, expression of this gene may be used as diagnostic marker to detect the presence of these cancers and also therapeutic modulation of this gene may be useful in the treatment of renal and breast cancers.

In addition, moderate to low levels of expression of this gene is also seen in fetal and adult liver, colon, stomach, pancreas, thymus, spleen, lymph node, and placenta. This gene codes for kynurenine hydroxylase, an enzyme in the tryptophan catabolism pathway. Tryptophan dioxygenase catalyzes the first step in the oxidative degradation of tryptophan, the dominant pathway for tryptophan catabolism. At Curagen, using GeneCalling studies it has been found that tryptophan dioxygenase was up-regulated in insulin-resistant (pre-diabetic) SHR vs normal WKY liver suggests Catabolic cleavage of the side chain of tryptophan yields the major gluconeogenic amino acid alanine. Increased intracellular levels of alanine could promote gluconeogenesis, increasing hepatic glucose production and blood glucose levels. Therefore, therapeutic inhibition of Kynurenine Hydroxylase, an enzyme in tryptophan catabolism pathway, would lead to 1) inhibit the excess production of glucose, thus ameliorating hyperglycemia in Type 2 diabetes, and 2) inhibit the synthesis of triglycerides, thus preventing excess weight gain.

Panel 5 Islet Summary: Ag4744/Ag6998 Low expression of this gene is restricted to placenta from diabetic and obese patients (CTs=32-33.9). Please see panel 1.4 for further discussion of this gene.

D. CG140122-03 and CG140122-04:Human Polyamine Oxidase-Like Protein.

Expression of gene CG140122-03 and CG140122-04 was assessed using the primer-probe sets Ag4986 and Ag5031, described in Tables DA and DB. Results of the RTQ-PCR runs are shown in Tables DC, DD and DE. Please note that probe-primer set is specific for CG140122-03. Also, CG140122-03 and CG140122-04 represent full length physical clone.

TABLE DA

Probe Name Ag4986

| | | | Start | SEQ ID |
|---|---|---|---|---|
| Primers | | Length | Position | No |
| Forward | 5′-gtgcagagtgtgaaacttgga-3′ | 21 | 194 | 500 |
| Probe | TET-5′-catggctcccatgggaaccctat-3′-TAMRA | 23 | 248 | 501 |
| Reverse | 5′-cgttggcttctgctagatgata-3′ | 22 | 272 | 502 |

TABLE DB

Probe Name Ag5031

| | | | Start | SEQ ID |
|---|---|---|---|---|
| Primers | | Length | Position | No |
| Forward | 5′-cggggtgtgctaaagag-3′ | 17 | 845 | 503 |
| Probe | TET-5′-cagtacaccagtttcttccggcca-3′-TAMRA | 24 | 863 | 504 |
| Reverse | 5′-accttctctgtgggcag-3′ | 17 | 890 | 505 |

TABLE DC
CNS_neurodegeneration_v1.0

| | Rel. | | Rel. |
|---|---|---|---|
| | Exp. (%) | | Exp. (%) |
| | Ag5031, | | Ag5031, |
| | Run | | Run |
| Tissue Name | 249286337 | Tissue Name | 249286337 |
| AD 1 Hippo | 37.1 | Control (Path) 3 Temporal Ctx | 21.9 |
| AD 2 Hippo | 53.2 | Control (Path) 4 Temporal Ctx | 24.7 |
| AD 3 Hippo | 15.0 | AD 1 Occipital Ctx | 27.7 |
| AD 4 Hippo | 24.5 | AD 2 Occipital Ctx (Missing) | 0.0 |
| AD 5 hippo | 59.5 | AD 3 Occipital Ctx | 24.0 |
| AD 6 Hippo | 100.0 | AD 4 Occipital Ctx | 18.6 |
| Control 2 Hippo | 31.0 | AD 5 Occipital Ctx | 20.7 |
| Control 4 Hippo | 42.3 | AD 6 Occipital Ctx | 24.1 |
| Control (Path) 3 Hippo | 12.5 | Control 1 Occipital Ctx | 12.5 |
| AD 1 Temporal Ctx | 35.4 | Control 2 Occipital Ctx | 39.2 |
| AD 2 Temporal Ctx | 36.9 | Control 3 Occipital Ctx | 26.2 |
| AD 3 Temporal Ctx | 22.5 | Control 4 Occipital Ctx | 19.8 |
| AD 4 Temporal Ctx | 24.3 | Control (Path) 1 Occipital Ctx | 40.9 |
| AD 5 Inf Temporal Ctx | 66.9 | Control (Path) 2 Occipital Ctx | 11.3 |
| AD 5 SupTemporal Ctx | 58.6 | Control (Path) 3 Occipital Ctx | 16.2 |
| AD 6 Inf Temporal Ctx | 75.3 | Control (Path) 4 Occipital Ctx | 12.7 |
| AD 6 Sup Temporal Ctx | 41.5 | Control 1 Parietal Ctx | 14.3 |
| Control 1 Temporal Ctx | 18.2 | Control 2 Parietal Ctx | 62.9 |
| Control 2 Temporal Ctx | 31.4 | Control 3 Parietal Ctx | 18.2 |
| Control 3 Temporal Ctx | 19.5 | Control (Path) 1 Parietal Ctx | 32.3 |
| Control 4 Temporal Ctx | 18.2 | Control (Path) 2 Parietal Ctx | 18.0 |
| Control (Path) 1 Temporal Ctx | 26.6 | Control (Path) 3 Parietal Ctx | 17.6 |
| Control (Path) 2 Temporal Ctx | 22.8 | Control (Path) 4 Parietal Ctx | 31.4 |

TABLE DD
General_screening_panel_v1.5

| | Rel. | Rel. |
|---|---|---|
| | Exp. (%) | Exp. (%) |
| | Ag5031, | Ag5031, |
| | Run | Run |
| Tissue Name | 228727243 | 228959437 |
| Adipose | 1.4 | 1.2 |
| Melanoma* Hs688(A).T | 3.0 | 3.1 |
| Melanoma* Hs688(B).T | 2.6 | 2.5 |
| Melanoma* M14 | 2.5 | 2.4 |
| Melanoma* LOXIMVI | 9.6 | 7.5 |
| Melanoma* SK-MEL-5 | 5.1 | 6.0 |
| Squamous cell carcinoma SCC-4 | 2.9 | 2.8 |
| Testis Pool | 1.3 | 1.3 |
| Prostate ca.* (bone met) PC-3 | 50.0 | 43.2 |
| Prostate Pool | 1.4 | 2.1 |
| Placenta | 0.4 | 0.8 |
| Uterus Pool | 1.0 | 0.7 |
| Ovarian ca. OVCAR-3 | 2.4 | 2.0 |
| Ovarian ca. SK-OV-3 | 7.1 | 9.5 |
| Ovarian ca. OVCAR-4 | 2.2 | 1.7 |
| Ovarian ca. OVCAR-5 | 14.8 | 14.8 |
| Ovarian ca. IGROV-1 | 7.3 | 0.9 |
| Ovarian ca. OVCAR-8 | 5.8 | 5.5 |
| Ovary | 1.0 | 1.6 |
| Breast ca. MCF-7 | 1.8 | 2.3 |
| Breast ca. MDA-MB-231 | 5.8 | 3.9 |
| Breast ca. BT 549 | 15.8 | 17.6 |
| Breast ca. T47D | 0.1 | 0.0 |
| Breast ca. MDA-N | 1.8 | 2.3 |
| Breast Pool | 2.0 | 2.1 |
| Trachea | 2.2 | 1.6 |
| Lung | 0.4 | 0.4 |
| Fetal Lung | 2.9 | 3.8 |
| Lung ca. NCI-N417 | 0.2 | 0.1 |
| Lung ca. LX-1 | 17.0 | 21.0 |
| Lung ca. NCI-H146 | 0.0 | 0.0 |
| Lung ca. SHP-77 | 1.2 | 0.6 |
| Lung ca. A549 | 46.0 | 29.1 |
| Lung ca. NCI-H526 | 1.9 | 1.9 |
| Lung ca. NCI-H23 | 2.8 | 3.3 |
| Lung ca. NCI-H460 | 100.0 | 100.0 |
| Lung ca. HOP-62 | 8.1 | 5.5 |
| Lung ca. NCI-H522 | 5.0 | 4.5 |
| Liver | 0.1 | 0.2 |
| Fetal Liver | 3.4 | 3.2 |
| Liver ca. HepG2 | 6.3 | 6.6 |
| Kidney Pool | 2.6 | 3.0 |
| Fetal Kidney | 1.7 | 2.8 |
| Renal ca. 786-0 | 12.9 | 14.5 |
| Renal ca. A498 | 2.0 | 2.7 |
| Renal ca. ACHN | 5.3 | 5.3 |
| Renal ca. UO-31 | 5.5 | 5.3 |
| Renal ca. TK-10 | 26.2 | 34.4 |
| Bladder | 3.1 | 2.9 |
| Gastric ca. (liver met.) NCI-N87 | 12.9 | 13.8 |
| Gastric ca. KATO III | 12.0 | 16.2 |
| Colon ca. SW-948 | 4.0 | 3.5 |
| Colon ca. SW480 | 12.7 | 11.0 |
| Colon ca.* (SW480 met) SW620 | 21.9 | 21.3 |
| Colon ca. HT29 | 2.4 | 5.8 |
| Colon ca. HCT-116 | 9.5 | 10.9 |
| Colon ca. CaCo-2 | 11.1 | 18.9 |
| Colon cancer tissue | 9.2 | 9.5 |
| Colon ca. SW1116 | 0.9 | 1.5 |
| Colon ca. Colo-205 | 5.5 | 5.8 |
| Colon ca. SW-48 | 4.8 | 4.6 |
| Colon Pool | 2.1 | 1.5 |
| Small Intestine Pool | 1.8 | 3.1 |
| Stomach Pool | 2.5 | 2.2 |
| Bone Marrow Pool | 1.0 | 1.1 |
| Fetal Heart | 1.1 | 1.0 |
| Heart Pool | 0.6 | 0.6 |
| Lymph Node Pool | 3.3 | 2.6 |
| Fetal Skeletal Muscle | 0.9 | 0.8 |
| Skeletal Muscle Pool | 1.2 | 1.5 |
| Spleen Pool | 0.9 | 0.9 |
| Thymus Pool | 2.3 | 2.7 |
| CNS cancer (glio/astro) U87-MG | 4.6 | 6.2 |
| CNS cancer (glio/astro) U-118-MG | 11.2 | 11.4 |
| CNS cancer (neuro; met) SK-N-AS | 1.4 | 1.8 |
| CNS cancer (astro) SF-539 | 1.8 | 1.8 |
| CNS cancer (astro) SNB-75 | 18.6 | 17.3 |
| CNS cancer (glio) SNB-19 | 11.8 | 16.3 |
| CNS cancer (glio) SF-295 | 16.5 | 18.0 |
| Brain (Amygdala) Pool | 6.1 | 5.6 |
| Brain (cerebellum) | 9.4 | 7.7 |
| Brain (fetal) | 5.8 | 6.2 |
| Brain (Hippocampus) Pool | 6.7 | 8.4 |
| Cerebral Cortex Pool | 7.1 | 6.2 |
| Brain (Substantia nigra) Pool | 7.2 | 8.6 |
| Brain (Thalamus) Pool | 8.9 | 7.9 |
| Brain (whole) | 5.7 | 5.8 |
| Spinal Cord Pool | 13.8 | 17.3 |
| Adrenal Gland | 1.1 | 0.8 |
| Pituitary gland Pool | 0.6 | 0.4 |
| Salivary Gland | 1.2 | 1.5 |
| Thyroid (female) | 0.7 | 0.7 |
| Pancreatic ca. CAPAN2 | 13.4 | 12.9 |
| Pancreas Pool | 2.8 | 2.9 |

TABLE DE
Panel 5D

| | Rel. | | Rel. |
|---|---|---|---|
| | Exp. (%) | | Exp. (%) |
| | Ag031, | | Ag5031, |
| | Run | | Run |
| Tissue Name | 223784820 | Tissue Name | 223784820 |
| 97457_Patient-02go_adipose | 24.5 | 94709_Donor 2 AM - A_adipose | 9.9 |
| 97476_Patient-07sk_skeletal | 2.1 | 94710_Donor 2 AM - B_adipose | 9.2 |
| muscle | | | |
| 97477_Patient-07ut_uterus | 3.5 | 94711_Donor 2 AM - C_adipose | 9.3 |
| 97478_Patient-07pl_placenta | 4.2 | 94712_Donor 2 AD - A_adipose | 12.2 |
| 97481_Patient-08sk_skeletal | 2.4 | 94713_Donor 2 AD - B_adipose | 16.0 |
| muscle | | | |
| 97482_Patient-08ut_uterus | 4.4 | 94714_Donor 2 AD - C_adipose | 18.6 |
| 97483_Patient-08pl_placenta | 1.4 | 94742_Donor 3 U - A_Mesenchymal | 14.6 |
| | | Stem Cells | |
| 97486_Patient-09sk_skeletal | 2.1 | 94743_Donor 3 U - B_Mesenchymal | 9.9 |
| muscle | | Stem Cells | |
| 97487_Patient-09ut_uterus | 3.6 | 94730_Donor 3 AM - A_adipose | 40.6 |
| 97488_Patient-09pl_placenta | 2.2 | 94731_Donor 3 AM - B_adipose | 24.1 |
| 97492_Patient-10ut_uterus | 10.0 | 94732_Donor 3 AM - C_adipose | 23.8 |
| 97493_Patient-10pl_placenta | 2.3 | 94733_Donor 3 AD - A_adipose | 31.0 |
| 97495_Patient-11go_adipose | 10.1 | 94734_Donor 3 AD - B_adipose | 16.5 |
| 97496_Patient-11sk_skeletal | 2.1 | 94735_Donor 3 AD - C_adipose | 24.5 |
| muscle | | | |
| 97497_Patient-11ut_uterus | 8.8 | 77138_Liver_HepG2untreated | 34.9 |
| 97498_Patient-11pl_placenta | 2.1 | 73556_Heart_Cardiac stromal cells | 4.8 |
| | | (primary) | |
| 97500_Patient-12go_adipose | 21.3 | 81735_Small Intestine | 3.1 |
| 97501_Patient-12sk_skeletal | 4.3 | 72409_Kidney_Proximal Convoluted | 17.6 |
| muscle | | Tubule | |
| 97502_Patient-12ut_uterus | 8.1 | 82685_Small intestine_Duodenum | 9.0 |
| 97503_Patient-12pl_placenta | 1.4 | 90650_Adrenal_Adrenocortical | 1.3 |
| | | adenoma | |
| 94721_Donor 2 U - | 21.2 | 72410_Kidney_HRCE | 100.0 |
| A_Mesenchymal Stem Cells | | | |
| 94722_Donor 2 U - | 18.3 | 72411_Kidney_HRE | 63.3 |
| B_Mesenchymal Stem Cells | | | |
| 94723_Donor 2 U - | 17.7 | 73139_Uterus_Uterine smooth | 8.7 |
| C_Mesenchymal Stem Cells | | muscle cells | |

CNS_neurodegeneration_v1.0 Summary: Ag5031 This panel confirms the expression of this gene at low levels in the brain in an independent group of individuals. This gene is found to be slighltly upregulated in the temporal cortex of Alzheimer's disease patients. Therefore, therapeutic modulation of the expression or function of this gene may decrease neuronal death and be of use in the treatment of this disease.

General_screening_panel_v1.5 Summary: Ag5031 Two experiments with same probe-primer sets are in good agreement with highest expression of this gene seen in a lung cancer NCI-H460 cell line (CTs=24-26). Moderate to high expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, fetal liver and the gastrointestinal tract. Therefore, therapeutic modulation of the activity of this gene may prove useful in the treatment of endocrine/metabolically related diseases, such as obesity and diabetes. This gene codes for polyamine oxidase (PAO, CG140122-01; BP24 obesity: CT021), an enzyme in the polyamine pathway. At Curagen, multiple enzymes in this pathway have been found to be up-regulated in GeneCalling studies upon adipose differentiation and are induced in obese mice versus obesity resistant mice on a high fat diet. Inhibiting polyamine catabolism and the synthesis of H2O2 through an inhibitor of PAO may abolish the insulin-like antilipolytic effects of polyamines and therefore be beneficial in the treatment of obesity.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CTs=31) when compared to adult lung and liver (CTs=34-35). This observation suggests that expression of this gene can be used to distinguish fetal from adult lung and liver. In addition, the relative overexpression of this gene in fetal tissue suggests that the protein product may enhance lung and liver growth or development in the fetus and thus may also act in a regenerative capacity in the adult. Therefore, therapeutic modulation of the protein encoded by this gene could be useful in treatment of lung and liver related diseases.

Panel 5D Summary: Ag5031 Highest expression of this gene is detected in kidney (CT=29.8). Moderate to low expression of this gene is seen mainly in undifferentiated and differentiated adipose, kidney, uterus and small intestine. Please see panel 1.5 for further discussion of this gene.

E. CG141051-01:Human Glyceraldehyde-Phosphate Deydrogenase-Like Protein.

Expression of gene CG141051-01 was assessed using the primer-probe set Ag5040, described in Table EA. Results of the RTQ-PCR runs are shown in Tables EB, EC, ED and EE.

TABLE EA

Probe Name AG5040

| | | | | |
|---|---|---|---|---|
| | | | Start | SEQ ID |
| Primers | Sequence | Length | Position | No |

| | | | | |
|---|---|---|---|---|
| Forward | 5′-cactcttccaccttcaatgct-3′ | 21 | 928 | 506 |
| Probe | TET-5′-ttgccctcaacaaccactttgtgaag-3′-TAMRA | 26 | 959 | 507 |
| Reverse | 5′-ctgttgctgtagccaaattca-3′ | 21 | 1005 | 508 |

TABLE EB
CNS_neurodegeneration_v1.0

| | Rel. | | Rel. |
|---|---|---|---|
| | Exp. (%) | | Exp. (%) |
| | Ag5040, | | Ag5040, |
| | Run | | Run |
| Tissue Name | 224062762 | Tissue Name | 224062762 |
| AD 1 Hippo | 22.1 | Control (Path) 3 Temporal Ctx | 8.7 |
| AD 2 Hippo | 44.8 | Control (Path) 4 Temporal Ctx | 87.7 |
| AD 3 Hippo | 9.2 | AD 1 Occipital Ctx | 24.3 |
| AD 4 Hippo | 20.2 | AD 2 Occipital Ctx (Missing) | 0.0 |
| AD 5 hippo | 81.2 | AD 3 Occipital Ctx | 3.1 |
| AD 6 Hippo | 90.8 | AD 4 Occipital Ctx | 29.9 |
| Control 2 Hippo | 31.6 | AD 5 Occipital Ctx | 10.4 |
| Control 4 Hippo | 16.8 | AD 6 Occipital Ctx | 29.7 |
| Control (Path) 3 Hippo | 9.8 | Control 1 Occipital Ctx | 1.7 |
| AD 1 Temporal Ctx | 28.1 | Control 2 Occipital Ctx | 34.2 |
| AD 2 Temporal Ctx | 53.2 | Control 3 Occipital Ctx | 40.1 |
| AD 3 Temporal Ctx | 13.1 | Control 4 Occipital Ctx | 5.4 |
| AD 4 Temporal Ctx | 43.8 | Control (Path) 1 Occipital Ctx | 82.4 |
| AD 5 Inf Temporal Ctx | 100.0 | Control (Path) 2 Occipital Ctx | 20.3 |
| AD 5 SupTemporal Ctx | 82.9 | Control (Path) 3 Occipital Ctx | 0.0 |
| AD 6 Inf Temporal Ctx | 71.7 | Control (Path) 4 Occipital Ctx | 45.7 |
| AD 6 Sup Temporal Ctx | 79.6 | Control 1 Parietal Ctx | 13.1 |
| Control 1 Temporal Ctx | 16.2 | Control 2 Parietal Ctx | 77.9 |
| Control 2 Temporal Ctx | 46.0 | Control 3 Parietal Ctx | 28.3 |
| Control 3 Temporal Ctx | 44.8 | Control (Path) 1 Parietal Ctx | 82.4 |
| Control 4 Temporal Ctx | 15.7 | Control (Path) 2 Parietal Ctx | 36.9 |
| Control (Path) 1 Temporal Ctx | 78.5 | Control (Path) 3 Parietal Ctx | 8.9 |
| Control (Path) 2 Temporal Ctx | 62.9 | Control (Path) 4 Parietal Ctx | 57.4 |

TABLE EC
General_screening_panel_v1.5

| | Rel. | | Rel. |
|---|---|---|---|
| | Exp. (%) | | Exp. (%) |
| | Ag5040, | | Ag5040, |
| | Run | | Run |
| Tissue Name | 228967325 | Tissue Name | 228967325 |
| Adipose | 1.8 | Renal ca. TK-10 | 16.3 |
| Melanoma* Hs688(A).T | 3.5 | Bladder | 9.6 |
| Melanoma* Hs688(B).T | 1.9 | Gastric ca. (liver met.) NCI-N87 | 18.4 |
| Melanoma* M14 | 10.6 | Gastric ca. KATO III | 10.2 |
| Melanoma* LOXIMVI | 2.4 | Colon ca. SW-948 | 3.8 |
| Melanoma* SK-MEL-5 | 50.3 | Colon ca. SW480 | 17.8 |
| Squamous cell carcinoma SCC-4 | 3.9 | Colon ca.* (SW480 met) SW620 | 28.3 |
| Testis Pool | 8.8 | Colon ca. HT29 | 5.6 |
| Prostate ca.* (bone met) PC-3 | 6.3 | Colon ca. HCT-116 | 19.8 |
| Prostate Pool | 24.0 | Colon ca. CaCo-2 | 26.2 |
| Placenta | 2.4 | Colon cancer tissue | 7.9 |
| Uterus Pool | 4.9 | Colon ca. SW1116 | 5.6 |
| Ovarian ca. OVCAR-3 | 9.2 | Colon ca. Colo-205 | 4.0 |
| Ovarian ca. SK-OV-3 | 19.5 | Colon ca. SW-48 | 1.8 |
| Ovarian ca. OVCAR-4 | 2.6 | Colon Pool | 16.6 |
| Ovarian ca. OVCAR-5 | 21.5 | Small Intestine Pool | 12.5 |
| Ovarian ca. IGROV-1 | 6.0 | Stomach Pool | 14.7 |
| Ovarian ca. OVCAR-8 | 3.1 | Bone Marrow Pool | 6.1 |
| Ovary | 5.8 | Fetal Heart | 1.8 |
| Breast ca. MCF-7 | 9.8 | Heart Pool | 6.0 |
| Breast ca. MDA-MB-231 | 12.5 | Lymph Node Pool | 24.5 |
| Breast ca. BT 549 | 9.5 | Fetal Skeletal Muscle | 3.8 |
| Breast ca. T47D | 3.4 | Skeletal Muscle Pool | 26.1 |
| Breast ca. MDA-N | 9.2 | Spleen Pool | 6.6 |
| Breast Pool | 16.6 | Thymus Pool | 16.5 |
| Trachea | 12.7 | CNS cancer (glio/astro) U87-MG | 34.9 |
| Lung | 6.1 | CNS cancer (glio/astro) U-118-MG | 21.3 |
| Fetal Lung | 28.5 | CNS cancer (neuro; met) SK-N-AS | 19.6 |
| Lung ca. NCI-N417 | 10.4 | CNS cancer (astro) SF-539 | 4.8 |
| Lung ca. LX-1 | 15.6 | CNS cancer (astro) SNB-75 | 21.3 |
| Lung ca. NCI-H146 | 1.5 | CNS cancer (glio) SNB-19 | 6.3 |
| Lung ca. SHP-77 | 13.5 | CNS cancer (glio) SF-295 | 22.8 |
| Lung ca. A549 | 11.2 | Brain (Amygdala) Pool | 6.4 |
| Lung ca. NCI-H526 | 2.4 | Brain (cerebellum) | 51.1 |
| Lung ca. NCI-H23 | 39.2 | Brain (fetal) | 100.0 |
| Lung ca. NCI-H460 | 22.4 | Brain (Hippocampus) Pool | 13.5 |
| Lung ca. HOP-62 | 0.0 | Cerebral Cortex Pool | 13.7 |
| Lung ca. NCI-H522 | 9.0 | Brain (Substantia nigra) Pool | 19.5 |
| Liver | 0.0 | Brain (Thalamus) Pool | 21.3 |
| Fetal Liver | 6.0 | Brain (whole) | 10.2 |
| Liver ca. HepG2 | 11.9 | Spinal Cord Pool | 5.4 |
| Kidney Pool | 18.7 | Adrenal Gland | 10.5 |
| Fetal Kidney | 20.3 | Pituitary gland Pool | 2.5 |
| Renal ca. 786-0 | 14.0 | Salivary Gland | 3.6 |
| Renal ca. A498 | 5.6 | Thyroid (female) | 3.0 |
| Renal ca. ACHN | 5.9 | Pancreatic ca. CAPAN2 | 8.5 |
| Renal ca. UO-31 | 14.8 | Pancreas Pool | 18.8 |

TABLE ED
Panel 4.1D

| | Rel. | | Rel. |
|---|---|---|---|
| | Exp. () | | Exp. (%) |
| | Ag5040, | | Ag5040, |
| | Run | | Run |
| Tissue Name | 223743486 | Tissue Name | 223743486 |
| Secondary Th1 act | 77.9 | HUVEC IL-1beta | 13.8 |
| Secondary Th2 act | 92.7 | HUVEC IFN gamma | 35.6 |
| Secondary Tr1 act | 54.0 | HUVEC TNF alpha + IFN gamma | 11.0 |
| Secondary Th1 rest | 6.2 | HUVEC TNF alpha + IL4 | 22.7 |
| Secondary Th2 rest | 27.2 | HUVEC IL-11 | 22.5 |
| Secondary Tr1 rest | 25.3 | Lung Microvascular EC none | 94.6 |
| Primary Th1 act | 38.4 | Lung Microvascular EC TNF alpha + IL-1beta | 40.6 |
| Primary Th2 act | 65.1 | Microvascular Dermal EC none | 15.5 |
| Primary Tr1 act | 62.4 | Microsvasular Dermal EC | 25.5 |
| | | TNF alpha + IL-1beta | |
| Primary Th1 rest | 23.3 | Bronchial epithelium TNF alpha + IL1beta | 27.5 |
| Primary Th2 rest | 15.9 | Small airway epithelium none | 8.0 |
| Primary Tr1 rest | 19.9 | Small airway epithelium TNF alpha + IL-1beta | 17.0 |
| CD45RA CD4 lymphocyte act | 31.0 | Coronery artery SMC rest | 11.0 |
| CD45RO CD4 lymphocyte act | 59.9 | Coronery artery SMC TNF alpha + IL-1beta | 10.8 |
| CD8 lymphocyte act | 50.0 | Astrocytes rest | 10.4 |
| Secondary CD8 lymphocyte rest | 30.8 | Astrocytes TNF alpha + IL-1beta | 18.4 |
| Secondary CD8 lymphocyte act | 15.4 | KU-812 (Basophil) rest | 56.3 |
| CD4 lymphocyte none | 14.7 | KU-812 (Basophil) | 57.4 |
| | | PMA/ionomycin | |
| 2ry Th1/Th2/Tr1_anti-CD95 | 16.0 | CCD1106 (Keratinocytes) none | 24.7 |
| CH11 | | | |
| LAK cells rest | 24.8 | CCD1106 (Keratinocytes) | 21.3 |
| | | TNF alpha + IL-1beta | |
| LAK cells IL-2 | 40.6 | Liver cirrhosis | 5.1 |
| LAK cells IL-2 + IL-12 | 29.7 | NCI-H292 none | 35.4 |
| LAK cells IL-2 + IFN gamma | 23.8 | NCI-H292 IL-4 | 31.9 |
| LAK cells IL-2 + IL-18 | 56.6 | NCI-H292 IL-9 | 61.6 |
| LAK cells PMA/ionomycin | 18.7 | NCI-H292 IL-13 | 24.7 |
| NK Cells IL-2 rest | 71.7 | NCI-H292 IFN gamma | 25.0 |
| Two Way MLR 3 day | 51.8 | HPAEC none | 14.8 |
| Two Way MLR 5 day | 25.3 | HPAEC TNF alpha + IL-1beta | 32.5 |
| Two Way MLR 7 day | 25.9 | Lung fibroblast none | 20.7 |
| PBMC rest | 8.6 | Lung fibroblast TNF alpha + | 7.2 |
| | | IL-1beta | |
| PBMC PWM | 25.3 | Lung fibroblast IL-4 | 14.1 |
| PBMC PHA-L | 28.1 | Lung fibroblast IL-9 | 13.9 |
| Ramos (B cell) none | 57.0 | Lung fibroblast IL-13 | 17.6 |
| Ramos (B cell) ionomycin | 61.1 | Lung fibroblast IFN gamma | 15.8 |
| B lymphocytes PWM | 22.1 | Dermal fibroblast CCD1070 rest | 71.2 |
| B lymphocytes CD40L and IL-4 | 47.3 | Dermal fibroblast CCD1070 | 60.3 |
| | | TNF alpha | |
| EOL-1 dbcAMP | 100.0 | Dermal fibroblast CCD1070 | 15.3 |
| | | IL-1beta | |
| EOL-1 dbcAMP | 69.7 | Dermal fibroblast IFN gamma | 10.2 |
| PMA/ionomycin | | | |
| Dendritic cells none | 46.3 | Dermal fibroblast IL-4 | 23.0 |
| Dendritic cells LPS | 16.4 | Dermal Fibroblasts rest | 9.9 |
| Dendritic cells anti-CD40 | 24.7 | Neutrophils TNF a + LPS | 0.0 |
| Monocytes rest | 31.2 | Neutrophils rest | 16.6 |
| Monocytes LPS | 45.1 | Colon | 4.2 |
| Macrophages rest | 41.8 | Lung | 12.0 |
| Macrophages LPS | 10.4 | Thymus | 52.9 |
| HUVEC none | 29.3 | Kidney | 57.0 |
| HUVEC starved | 21.2 | | |

TABLE EE
Panel 5 Islet

| | Rel. | | Rel. |
|---|---|---|---|
| | Exp. () | | Exp. (%) |
| | Ag5040, | | Ag5040, |
| | Run | | Run |
| Tissue Name | 240189534 | Tissue Name | 240189534 |
| 97457_Patient-02go_adipose | 11.6 | 94709_Donor 2 AM - A_adipose | 0.0 |
| 97476_Patient-07sk_skeletal | 16.3 | 94710_Donor 2 AM - B_adipose | 4.1 |
| muscle | | | |
| 97477_Patient-07ut_uterus | 5.7 | 94711_Donor 2 AM - C_adipose | 0.0 |
| 97478_Patient-07pl_placenta | 17.6 | 94712_Donor 2 AD - A_adipose | 6.5 |
| 99167_Bayer Patient 1 | 100.0 | 94713_Donor 2 AD - B_adipose | 6.8 |
| 97482_Patient-08ut_uterus | 0.0 | 94714_Donor 2 AD - C_adipose | 2.5 |
| 97483_Patient-08pl_placenta | 23.5 | 94742_Donor 3 U - A_Mesenchymal | 0.0 |
| | | Stem Cells | |
| 97486_Patient-09sk_skeletal | 2.8 | 94743_Donor 3 U - B_Mesenchymal | 0.0 |
| muscle | | Stem Cells | |
| 97487_Patient-09ut_uterus | 9.2 | 94730_Donor 3 AM - A_adipose | 3.1 |
| 97488_Patient-09pl_placenta | 6.9 | 94731_Donor 3 AM - B_adipose | 6.5 |
| 97492_Patient-10ut_uterus | 11.4 | 94732_Donor 3 AM - C_adipose | 0.0 |
| 97493_Patient-10pl_placenta | 12.2 | 94733_Donor 3 AD - A_adipose | 0.0 |
| 97495_Patient-11go_adipose | 15.2 | 94734_Donor 3 AD - B_adipose | 3.6 |
| 97496_Patient-11sk_skeletal | 4.4 | 94735_Donor 3 AD - C_adipose | 7.7 |
| muscle | | | |
| 97497_Patient-11ut_uterus | 10.1 | 77138_Liver_HepG2untreated | 12.7 |
| 97498_Patient-11pl_placenta | 0.0 | 73556_Heart_Cardiac stromal cells | 6.8 |
| | | (primary) | |
| 97500_Patient-12go_adipose | 10.7 | 81735_Small Intestine | 13.4 |
| 97501_Patient-12sk_skeletal | 21.6 | 72409_Kidney_Proximal Convoluted | 9.9 |
| muscle | | Tubule | |
| 97502_Patient-12ut_uterus | 0.0 | 82685_Small intestine_Duodenum | 0.0 |
| 97503_Patient-12pl_placenta | 2.4 | 90650_Adrenal_Adrenocortical | 0.0 |
| | | adenoma | |
| 94721_Donor 2 U - | 3.7 | 72410_Kidney_HRCE | 20.2 |
| A_Mesenchymal Stem Cells | | | |
| 94722_Donor 2 U - | 0.0 | 72411_Kidney_HRE | 0.0 |
| B_Mesenchymal Stem Cells | | | |
| 94723_Donor 2 U - | 0.0 | 73139_Uterus_Uterine smooth | 4.6 |
| C_Mesenchymal Stem Cells | | muscle cells | |

CNS_neurodegeneration_v1.0 Summary: Ag5040 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of this gene in treatment of central nervous system disorders.

General_screening_panel_v1.5 Summary: Ag5040 Highest expression of this gene is detected in fetal brain (CT=30.8). This gene is expressed at low levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Among tissues with metabolic or endocrine function, this gene is expressed at low levels in pancreas, adrenal gland, skeletal muscle, heart, fetal liver and the gastrointestinal tract. Therefore, therapeutic modulation of the activity of this gene may prove useful in the treatment of endocrine/metabolically related diseases, such as obesity and diabetes.

In addition, low expression of this gene is also seen in a number of cancer cell lines derived from brain, pancreatic, colon, renal, liver, lung, melanoma, breast, ovarian and prostate cancers. Therefore, therapeutic modulation of the expression or function of this gene may be effective in the treatment of pancreatic, gastric, colon, lung, liver, renal, breast, ovarian, prostate, melanoma and brain cancers.

Panel 4.1D Summary: Ag5040 Highest expression of this gene is detected in eosinophils (CT=33.4). Low expression of this gene is detected in activated polarized T cells, memory T cells, activated LAK cells, IL-2 treated resting NK cells, monocytes, macrophage, lung microvascular endothelial cells, basophils, dermal fibroblast and normal tissues represented by thymus and kidney. Therefore, therapeutic modulation of this gene may be useful in the treatment of inflammatory and autoimmune diseases including asthma, allergies, inflammatory bowel disease, lupus erythematosus, psoriasis, rheumatoid arthritis, and osteoarthritis.

Panel 5 Islet Summary: Ag5040 Low expression of this gene is restricted to islet cells. Therefore, therapeutic modulation of this gene may be useful in the treatment of obesity and diabetes especially Type II diabetes.

F. CG142427-03 and CG142427-04: Human ATP-Citrate (pro-S-)-Lyase-Like Protein

Expression of gene CG142427-03 and CG142427-04 was assessed using the primer-probe sets Ag6008, Ag6980 and Ag7002, described in Tables FA, FB and FC. Results of the RTQ-PCR runs are shown in Tables FD, FE and FF. Please note that Ag6980 is specific for CG142427-03. Also, CG142427-03 and CG142427-04 represent full length physical clone.

TABLE FA

Probe Name Ag6008

| | | | Start | SEQ ID |
|---|---|---|---|---|
| Primers | Sequence | Length | Position | No |
| Forward | 5′-agattacgtcaggcagcactt-3′ | 21 | 1939 | 509 |
| Probe | TET-5′-cactcctctgctcgattatgcactgg-+-TAMRA | 26 | 1966 | 510 |
| Reverse | 5′-gcttcttcgaggtggtaatctt-3′ | 22 | 2000 | 511 |

TABLE FB

Probe Name Ag6980

| | | | Start | SEQ ID |
|---|---|---|---|---|
| Primers | Sequence | Length | Position | No |
| Forward | 5′-aagatgaacgtgtgtggtaacag-3′ | 23 | 314 | 512 |
| Probe | TET-5′-ccttgccaacctgaaggtgaccatat-3′-TAMRA | 26 | 343 | 513 |
| Reverse | 5′-cgatcagaaagttcttgaggaa-3′ | 22 | 377 | 514 |

TABLE FC

Probe Name Ag7002

| | | | Start | SEQ ID |
|---|---|---|---|---|
| Primers | Sequence | Length | Position | No |
| Forward | 5′-ccatgccacaaggaaagag-3′ | 19 | 1494 | 515 |
| Probe | TET-5′-tcaaagtccagcatgccttgcacgg-3′-TAMRA | 25 | 1569 | 516 |
| Reverse | 5′-cgtctcgggagcagacata-3′ | 19 | 1595 | 517 |

TABLE FD
General_screening_panel_v1.5

| | Rel. | | Rel. |
|---|---|---|---|
| | Exp. (%) | | Exp. (%) |
| | Ag6008, | | Ag6008, |
| | Run | | Run |
| Tissue Name | 228763479 | Tissue Name | 228763479 |
| Adipose | 6.2 | Renal ca. TK-10 | 64.2 |
| Melanoma* Hs688(A).T | 37.6 | Bladder | 12.4 |
| Melanoma* Hs688(B).T | 59.0 | Gastric ca. (liver met.) NCI-N87 | 65.1 |
| Melanoma* M14 | 55.9 | Gastric ca. KATO III | 59.5 |
| Melanoma* LOXIMVI | 59.0 | Colon ca. SW-948 | 14.5 |
| Melanoma* SK-MEL-5 | 41.8 | Colon ca. SW480 | 62.4 |
| Squamous cell carcinoma SCC-4 | 24.1 | Colon ca.* (SW480 met) SW620 | 32.3 |
| Testis Pool | 6.0 | Colon ca. HT29 | 27.4 |
| Prostate ca.* (bone met) PC-3 | 32.8 | Colon ca. HCT-116 | 45.7 |
| Prostate Pool | 13.0 | Colon ca. CaCo-2 | 66.0 |
| Placenta | 6.1 | Colon cancer tissue | 8.3 |
| Uterus Pool | 6.6 | Colon ca. SW1116 | 4.0 |
| Ovarian ca. OVCAR-3 | 12.9 | Colon ca. Colo-205 | 11.1 |
| Ovarian ca. SK-OV-3 | 47.3 | Colon ca. SW-48 | 14.9 |
| Ovarian ca. OVCAR-4 | 17.2 | Colon Pool | 13.3 |
| Ovarian ca. OVCAR-5 | 35.1 | Small Intestine Pool | 5.6 |
| Ovarian ca. IGROV-1 | 22.2 | Stomach Pool | 4.0 |
| Ovarian ca. OVCAR-8 | 8.2 | Bone Marrow Pool | 3.8 |
| Ovary | 8.0 | Fetal Heart | 3.5 |
| Breast ca. MCF-7 | 23.7 | Heart Pool | 2.5 |
| Breast ca. MDA-MB-231 | 46.7 | Lymph Node Pool | 8.4 |
| Breast ca. BT 549 | 60.7 | Fetal Skeletal Muscle | 3.7 |
| Breast ca. T47D | 29.1 | Skeletal Muscle Pool | 3.4 |
| Breast ca. MDA-N | 12.9 | Spleen Pool | 5.3 |
| Breast Pool | 8.0 | Thymus Pool | 6.8 |
| Trachea | 9.3 | CNS cancer (glio/astro) U87-MG | 60.7 |
| Lung | 1.4 | CNS cancer (glio/astro) U-118-MG | 59.0 |
| Fetal Lung | 16.3 | CNS cancer (neuro; met) SK-N-AS | 60.7 |
| Lung ca. NCI-N417 | 30.1 | CNS cancer (astro) SF-539 | 24.8 |
| Lung ca. LX-1 | 28.1 | CNS cancer (astro) SNB-75 | 32.5 |
| Lung ca. NCI-H146 | 23.5 | CNS cancer (glio) SNB-19 | 25.2 |
| Lung ca. SHP-77 | 46.7 | CNS cancer (glio) SF-295 | 76.8 |
| Lung ca. A549 | 100.0 | Brain (Amygdala) Pool | 4.8 |
| Lung ca. NCI-H526 | 10.0 | Brain (cerebellum) | 28.3 |
| Lung ca. NCI-H23 | 23.5 | Brain (fetal) | 16.5 |
| Lung ca. NCI-H460 | 25.5 | Brain (Hippocampus) Pool | 8.6 |
| Lung ca. HOP-62 | 29.5 | Cerebral Cortex Pool | 10.5 |
| Lung ca. NCI-H522 | 57.4 | Brain (Substantia nigra) Pool | 6.3 |
| Liver | 0.8 | Brain (Thalamus) Pool | 10.7 |
| Fetal Liver | 22.4 | Brain (whole) | 12.2 |
| Liver ca. HepG2 | 23.0 | Spinal Cord Pool | 7.4 |
| Kidney Pool | 7.5 | Adrenal Gland | 13.2 |
| Fetal Kidney | 5.4 | Pituitary gland Pool | 1.9 |
| Renal ca. 786-0 | 36.3 | Salivary Gland | 4.0 |
| Renal ca. A498 | 33.0 | Thyroid (female) | 2.7 |
| Renal ca. ACHN | 80.7 | Pancreatic ca. CAPAN2 | 36.3 |
| Renal ca. UO-31 | 31.9 | Pancreas Pool | 11.2 |

TABLE FE
General_screening_panel_v1.6

| | Rel. | Rel. |
|---|---|---|
| | Exp. (%) | Exp. (%) |
| | Ag6980, | Ag7002, |
| | Run | Run |
| Tissue Name | 279065836 | 279066491 |
| Adipose | 3.7 | 1.4 |
| Melanoma* Hs688(A).T | 4.3 | 42.9 |
| Melanoma* Hs688(B).T | 16.3 | 56.3 |
| Melanoma* M14 | 5.6 | 29.1 |
| Melanoma* LOXIMVI | 21.3 | 29.3 |
| Melanoma* SK-MEL-5 | 26.1 | 13.7 |
| Squamous cell carcinoma SCC-4 | 4.5 | 15.5 |
| Testis Pool | 4.0 | 3.0 |
| Prostate ca.* (bone met) PC-3 | 8.4 | 11.0 |
| Prostate Pool | 0.7 | 10.7 |
| Placenta | 0.8 | 6.8 |
| Uterus Pool | 0.0 | 0.9 |
| Ovarian ca. OVCAR-3 | 0.9 | 4.2 |
| Ovarian ca. SK-OV-3 | 41.5 | 43.8 |
| Ovarian ca. OVCAR-4 | 7.7 | 1.8 |
| Ovarian ca. OVCAR-5 | 52.9 | 9.0 |
| Ovarian ca. IGROV-1 | 19.3 | 22.4 |
| Ovarian ca. OVCAR-8 | 0.9 | 7.6 |
| Ovary | 6.0 | 5.3 |
| Breast ca. MCF-7 | 13.7 | 6.4 |
| Breast ca. MDA-MB-231 | 27.5 | 24.7 |
| Breast ca. BT 549 | 15.1 | 51.4 |
| Breast ca. T47D | 13.6 | 2.3 |
| Breast ca. MDA-N | 2.6 | 10.1 |
| Breast Pool | 2.8 | 5.3 |
| Trachea | 3.9 | 6.2 |
| Lung | 0.0 | 0.2 |
| Fetal Lung | 4.4 | 6.3 |
| Lung ca. NCI-N417 | 3.4 | 13.3 |
| Lung ca. LX-1 | 19.1 | 6.1 |
| Lung ca. NCI-H146 | 6.0 | 3.9 |
| Lung ca. SHP-77 | 62.4 | 25.9 |
| Lung ca. A549 | 100.0 | 100.0 |
| Lung ca. NCI-H526 | 8.8 | 1.8 |
| Lung ca. NCI-H23 | 2.5 | 18.4 |
| Lung ca. NCI-H460 | 8.8 | 4.9 |
| Lung ca. HOP-62 | 3.8 | 24.7 |
| Lung ca. NCI-H522 | 8.2 | 14.4 |
| Liver | 0.0 | 0.7 |
| Fetal Liver | 7.5 | 13.8 |
| Liver ca. HepG2 | 20.0 | 10.7 |
| Kidney Pool | 0.5 | 5.2 |
| Fetal Kidney | 1.3 | 1.9 |
| Renal ca. 786-0 | 6.7 | 31.0 |
| Renal ca. A498 | 6.3 | 21.3 |
| Renal ca. ACHN | 55.9 | 27.9 |
| Renal ca. UO-31 | 18.4 | 31.0 |
| Renal ca. TK-10 | 52.1 | 33.9 |
| Bladder | 1.6 | 6.0 |
| Gastric ca. (liver met.) NCI-N87 | 9.4 | 16.6 |
| Gastric ca. KATO III | 25.3 | 25.9 |
| Colon ca. SW-948 | 5.3 | 7.9 |
| Colon ca. SW480 | 43.2 | 20.3 |
| Colon ca.* (SW480 met) SW620 | 21.3 | 9.3 |
| Colon ca. HT29 | 14.5 | 9.3 |
| Colon ca. HCT-116 | 34.9 | 43.8 |
| Colon ca. CaCo-2 | 40.6 | 28.9 |
| Colon cancer tissue | 1.1 | 6.2 |
| Colon ca. SW1116 | 5.2 | 0.9 |
| Colon ca. Colo-205 | 14.1 | 2.0 |
| Colon ca. SW-48 | 7.9 | 6.4 |
| Colon Pool | 2.2 | 4.5 |
| Small Intestine Pool | 0.8 | 1.9 |
| Stomach Pool | 2.3 | 2.4 |
| Bone Marrow Pool | 0.0 | 1.3 |
| Fetal Heart | 0.0 | 1.4 |
| Heart Pool | 3.2 | 1.5 |
| Lymph Node Pool | 3.2 | 5.5 |
| Fetal Skeletal Muscle | 2.5 | 0.9 |
| Skeletal Muscle Pool | 0.0 | 0.2 |
| Spleen Pool | 2.7 | 3.3 |
| Thymus Pool | 0.5 | 2.7 |
| CNS cancer (glio/ astro) U87-MG | 13.7 | 29.9 |
| CNS cancer (glio/ astro) U-118-MG | 34.9 | 31.2 |
| CNS cancer (neuro; met) SK-N-AS | 57.0 | 48.6 |
| CNS cancer (astro) SF-539 | 18.0 | 20.2 |
| CNS cancer (astro) SNB-75 | 22.4 | 40.9 |
| CNS cancer (glio) SNB-19 | 10.7 | 22.4 |
| CNS cancer (glio) SF-295 | 30.1 | 38.2 |
| Brain (Amygdala) Pool | 1.0 | 4.7 |
| Brain (cerebellum) | 18.6 | 13.3 |
| Brain (fetal) | 18.0 | 12.2 |
| Brain (Hippocampus) Pool | 0.5 | 3.6 |
| Cerebral Cortex Pool | 3.8 | 4.0 |
| Brain (Substantia nigra) Pool | 3.9 | 3.9 |
| Brain (Thalamus) Pool | 4.3 | 5.5 |
| Brain (whole) | 5.9 | 4.9 |
| Spinal Cord Pool | 4.4 | 13.8 |
| Adrenal Gland | 3.9 | 9.3 |
| Pituitary gland Pool | 0.3 | 0.8 |
| Salivary Gland | 2.1 | 3.4 |
| Thyroid (female) | 0.0 | 3.0 |
| Pancreatic ca. CAPAN2 | 15.5 | 12.0 |
| Pancreas Pool | 3.6 | 3.0 |

TABLE FF
Panel 5 Islet

| | Rel. | Rel. | Rel. |
|---|---|---|---|
| | Exp. (%) | Exp. (%) | Exp. (%) |
| | Ag6008, | Ag6980, | Ag7002, |
| | Run | Run | Run |
| Tissue Name | 245239907 | 284710391 | 284710397 |
| 97457_Patient-02go_{—} | 12.6 | 9.2 | 1.4 |
| adipose | | | |
| 97476_Patient-07sk_{—} | 9.5 | 0.0 | 0.0 |
| skeletal muscle | | | |
| 97477_Patient-07ut_{—} | 8.4 | 0.0 | 3.5 |
| uterus | | | |
| 97478_Patient-07pl_{—} | 16.4 | 0.0 | 2.5 |
| placenta | | | |
| 99167_Bayer Patient 1 | 70.7 | 0.0 | 0.0 |
| 97482_Patient-08ut_{—} | 7.9 | 0.0 | 1.1 |
| uterus | | | |
| 97483_Patient-08pl_{—} | 15.6 | 2.4 | 0.6 |
| placenta | | | |
| 97486_Patient-09sk_{—} | 0.6 | 0.0 | 0.9 |
| skeletal muscle | | | |
| 97487_Patient-09ut_{—} | 3.6 | 0.0 | 1.2 |
| uterus | | | |
| 97488_Patient-09pl_{—} | 9.6 | 0.0 | 1.5 |
| placenta | | | |
| 97492_Patient-10ut_{—} | 9.9 | 0.0 | 2.2 |
| uterus | | | |
| 97493_Patient-10pl_{—} | 18.3 | 0.0 | 2.4 |
| placenta | | | |
| 97495_Patient-11go_{—} | 5.5 | 0.0 | 1.2 |
| adipose | | | |
| 97496_Patient-11sk_{—} | 0.4 | 0.0 | 0.7 |
| skeletal muscle | | | |
| 97497_Patient-11ut_{—} | 3.5 | 2.5 | 2.3 |
| uterus | | | |
| 97498_Patient-11pl_{—} | 11.0 | 4.5 | 1.4 |
| placenta | | | |
| 97500_Patient-12go_{—} | 7.4 | 0.0 | 3.3 |
| adipose | | | |
| 97501_Patient-12sk_{—} | 6.9 | 4.1 | 1.3 |
| skeletal muscle | | | |
| 97502_Patient-12ut_{—} | 9.3 | 0.0 | 4.5 |
| uterus | | | |
| 97503_Patient-12pl_{—} | 6.1 | 6.7 | 4.9 |
| placenta | | | |
| 94721_Donor 2 U - A_{—} | 6.7 | 2.6 | 25.5 |
| Mesenchymal Stem Cells | | | |
| 94722_Donor 2 U - B_{—} | 13.6 | 0.0 | 21.5 |
| Mesenchymal Stem Cells | | | |
| 94723_Donor 2 U - C_{—} | 8.9 | 11.9 | 29.1 |
| Mesenchymal Stem Cells | | | |
| 94709_Donor 2 AM - A_{—} | 26.8 | 3.9 | 25.7 |
| adipose | | | |
| 94710_Donor 2 AM - B_{—} | 26.4 | 0.0 | 23.2 |
| adipose | | | |
| 94711_Donor 2 AM - C_{—} | 8.4 | 3.4 | 14.4 |
| adipose | | | |
| 94712_Donor 2 AD - A_{—} | 37.6 | 4.6 | 36.6 |
| adipose | | | |
| 94713_Donor 2 AD - B_{—} | 31.0 | 19.8 | 68.3 |
| adipose | | | |
| 94714_Donor 2 AD - C_{—} | 59.0 | 7.3 | 35.4 |
| adipose | | | |
| 94742_Donor 3 U - A_{—} | 11.0 | 5.6 | 10.9 |
| Mesenchymal Stem Cells | | | |
| 94743_Donor 3 U - B_{—} | 34.2 | 9.3 | 11.3 |
| Mesenchymal Stem Cells | | | |
| 94730_Donor 3 AM - A_{—} | 60.3 | 37.6 | 43.2 |
| adipose | | | |
| 94731_Donor 3 AM - B_{—} | 27.4 | 38.4 | 71.7 |
| adipose | | | |
| 94732_Donor 3 AM - C_{—} | 42.3 | 22.7 | 50.7 |
| adipose | | | |
| 94733_Donor 3 AD - A_{—} | 100.0 | 100.0 | 100.0 |
| adipose | | | |
| 94734_Donor 3 AD - B_{—} | 44.1 | 54.3 | 90.8 |
| adipose | | | |
| 94735_Donor 3 AD - C_{—} | 84.1 | 25.3 | 30.1 |
| adipose | | | |
| 77138_Liver_HepG2 | 0.0 | 61.1 | 20.9 |
| untreated | | | |
| 73556_Heart_Cardiac | 14.8 | 4.9 | 3.2 |
| stromal cells (primary) | | | |
| 81735_Small Intestine | 9.5 | 0.0 | 2.8 |
| 72409_Kidney_Proximal | 24.5 | 4.8 | 8.1 |
| Convoluted Tubule | | | |
| 82685_Small intestine_{—} | 7.1 | 0.0 | 2.9 |
| Duodenum | | | |
| 90650_Adrenal_Adreno | 2.4 | 0.0 | 0.6 |
| cortical adenoma | | | |
| 72410_Kidney_HRCE | 65.5 | 16.7 | 20.3 |
| 72411_Kidney_HRE | 46.0 | 4.8 | 9.4 |
| 73139_Uterus_Uterine | 30.4 | 3.5 | 11.6 |
| smooth muscle cells | | | |

General_screening_panel_v1.5 Summary: Ag6008 Highest expression of this gene is detected in a lung cancer A549 cell line (CT=22.4). High expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at high levels in pancreas, adipose, adrenal gland, thyroid, pituitary gland, skeletal muscle, heart, liver and the gastrointestinal tract. Therefore, therapeutic modulation of the activity of this gene through the use of small molecule drug may prove useful in the treatment of endocrine/metabolically related diseases, such as obesity and diabetes.

Interestingly, this gene is expressed at much higher levels in fetal (CTs=24-25), when compared to adult liver and lung (CTs=28-29). This observation suggests that expression of this gene can be used to distinguish fetal from adult lung and liver. In addition, the relative overexpression of this gene in fetal tissue suggests that the protein product may enhance lung and liver growth or development in the fetus and thus may also act in a regenerative capacity in the adult. Therefore, therapeutic modulation of the protein encoded by this gene could be useful in treatment of lung and liver related diseases.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.6 Summary: Ag6980/Ag7002 Highest expression of this gene is detected in a lung cancer A549 cell line (CT=24.3). The expression profile in this panel correlates with the pattern seen in panel 1.5. Please see panel 1.5 for further discussion of this gene.

Panel 5 Islet Summary: Ag6008/Ag6980/Ag7002 Highest expression of this gene is detected in differentiated adipose (CTs=27-33.7). Expression of this gene is higher in undifferentiated, midway differentiated and differentiated adipose tissue. Moderate to low expression of this gene is detected in the tissues with metabolic/endocrine functions including islet cells, adipose, skeletal muscle, and gastrointestinal tracts.

This gene codes for ATP-citrate lyase. It is a major source of acetyl CoA that is the building block of lipid biosynthesis and provides substrate for the production of cholesterol. Reduced flux of acetyl CoA through the cholesterol biosynthetic pathway will prevent excess production of LXR alpha ligands. LXR alpha is a nuclear hormone receptor that is abundantly expressed in tissues associated with lipid metabolism. Activation of LXR alpha leads to the up-regulation of fatty acid synthesis. Thus, ATP-citrate lyase may be a target for the treatment and/or prevention of obesity because its inhibition will decrease the availability of acetyl CoA for the synthesis of LXR alpha ligands, fatty acids, and triglycerides.

Chawla A, Repa J J, Evans R M, Mangelsdorf D J. Nuclear receptors and lipid physiology: opening the X-files. Science. Nov. 30, 2001;294(5548):1866-70. Review. PMID: 11729302; Moon Y A, Lee J J, Park S W, Ahn Y H, Kim K S. The roles of sterol regulatory element-binding proteins in the transactivation of the rat ATP citrate-lyase promoter. J Biol Chem. Sep. 29, 2000;275(39):30280-6. PMID: 10801800; Sato R, Okamoto A, Inoue J, Miyamoto W, Sakai Y, Emoto N, Shimano H, Maeda M. Transcriptional regulation of the ATP citrate-lyase gene by sterol regulatory element-binding proteins. J Biol Chem. Apr. 28, 2000;275(17): 12497-502. PMID: 10777536.

G. CG148010-01: Human Dacylglycerol Acyltransferase 2-Like Protein.

Expression of gene CG148010-01 was assessed using the primer-probe set Ag6056, described in Table GA. Results of the RTQ-PCR runs are shown in Tables GB and GC.

TABLE GA

Probe Name Ag6056

| | | | Start | SEQ ID |
|---|---|---|---|---|
| Primers | Sequence | Length | Position | No |
| Forward | 5′-ccagaagaagttccagaaataca-3′ | 24 | 0 | 377 |
| Probe | TET-5′-atcttccatggtcgaggcctcttctcc-3′-TAMRA | 28 | 0 | 378 |
| Reverse | 5′-gtggtgatgggcttggagta-3′ | 21 | 0 | 379 |

TABLE GB
General_screening_panel_v1.5

| | Rel. | | Rel. |
|---|---|---|---|
| | Exp. (%) | | Exp. (%) |
| | Ag6056, | | Ag6056, |
| | Run | | Run |
| Tissue Name | 229514475 | Tissue Name | 229514475 |
| Adipose | 40.1 | Renal ca. TK-10 | 19.6 |
| Melanoma* Hs688(A).T | 0.7 | Bladder | 2.9 |
| Melanoma* Hs688(B).T | 3.2 | Gastric ca. (liver met.) NCI-N87 | 28.9 |
| Melanoma* M14 | 3.1 | Gastric ca. KATO III | 37.1 |
| Melanoma* LOXIMVI | 0.2 | Colon ca. SW-948 | 12.2 |
| Melanoma* SK-MEL-5 | 4.7 | Colon ca. SW480 | 6.2 |
| Squamous cell carcinoma SCC-4 | 0.2 | Colon ca.* (SW480 met) SW620 | 10.4 |
| Testis Pool | 7.7 | Colon ca. HT29 | 11.0 |
| Prostate ca.* (bone met) PC-3 | 6.8 | Colon ca. HCT-116 | 3.3 |
| Prostate Pool | 0.9 | Colon ca. CaCo-2 | 100.0 |
| Placenta | 0.3 | Colon cancer tissue | 12.8 |
| Uterus Pool | 0.3 | Colon ca. SW1116 | 3.3 |
| Ovarian ca. OVCAR-3 | 16.3 | Colon ca. Colo-205 | 9.5 |
| Ovarian ca. SK-OV-3 | 3.5 | Colon ca. SW-48 | 23.3 |
| Ovarian ca. OVCAR-4 | 3.0 | Colon Pool | 0.3 |
| Ovarian ca. OVCAR-5 | 6.1 | Small Intestine Pool | 0.8 |
| Ovarian ca. IGROV-1 | 4.5 | Stomach Pool | 0.6 |
| Ovarian ca. OVCAR-8 | 8.4 | Bone Marrow Pool | 0.9 |
| Ovary | 1.7 | Fetal Heart | 3.1 |
| Breast ca. MCF-7 | 20.4 | Heart Pool | 1.1 |
| Breast ca. MDA-MB-231 | 9.7 | Lymph Node Pool | 0.1 |
| Breast ca. BT 549 | 18.7 | Fetal Skeletal Muscle | 7.0 |
| Breast ca. T47D | 0.8 | Skeletal Muscle Pool | 2.3 |
| Breast ca. MDA-N | 7.2 | Spleen Pool | 1.0 |
| Breast Pool | 0.4 | Thymus pool | 3.0 |
| Trachea | 7.3 | CNS cancer (glio/astro) U87-MG | 6.2 |
| Lung | 0.2 | CNS cancer (glio/astro) U-118-MG | 12.9 |
| Fetal Lung | 2.4 | CNS cancer (neuro; met) SK-N-AS | 0.4 |
| Lung ca. NCI-N417 | 6.0 | CNS cancer (astro) SF-539 | 0.2 |
| Lung ca. LX-1 | 10.6 | CNS cancer (astro) SNB-75 | 15.2 |
| Lung ca. NCI-H146 | 3.2 | CNS cancer (glio) SNB-19 | 6.3 |
| Lung ca. SHP-77 | 0.3 | CNS cancer (glio) SF-295 | 15.8 |
| Lung ca. A549 | 0.8 | Brain (Amygdala) Pool | 3.2 |
| Lung ca. NCI-H526 | 7.1 | Brain (cerebellum) | 1.5 |
| Lung ca. NCI-H23 | 48.6 | Brain (fetal) | 3.1 |
| Lung ca. NCI-H460 | 2.8 | Brain (Hippocampus) Pool | 3.2 |
| Lung ca. HOP-62 | 1.0 | Cerebral Cortex Pool | 4.2 |
| Lung ca. NCI-H522 | 9.2 | Brain (Substantia nigra) Pool | 3.5 |
| Liver | 42.6 | Brain (Thalamus) Pool | 4.4 |
| Fetal Liver | 71.7 | Brain (whole) | 6.1 |
| Liver ca. HepG2 | 34.2 | Spinal Cord Pool | 1.5 |
| Kidney Pool | 0.9 | Adrenal Gland | 7.6 |
| Fetal Kidney | 1.0 | Pituitary gland Pool | 0.2 |
| Renal ca. 786-0 | 0.7 | Salivary Gland | 2.9 |
| Renal ca. A498 | 1.6 | Thyroid (female) | 4.1 |
| Renal ca. ACHN | 0.6 | Pancreatic ca. CAPAN2 | 7.6 |
| Renal ca. UO-31 | 0.6 | Pancreas Pool | 0.7 |

TABLE GC
Panel 5 Islet

| | Rel. | | Rel. |
|---|---|---|---|
| | Exp. (%) | | Exp. (%) |
| | Ag6056, | | Ag6056, |
| | Run | | Run |
| Tissue Name | 230294205 | Tissue Name | 230294205 |
| 97457_Patient-02go_adipose | 11.5 | 94709_Donor 2 AM - A_adipose | 24.5 |
| 97476_Patient-07sk_skeletal | 3.8 | 94710_Donor 2 AM - B_adipose | 12.8 |
| muscle | | | |
| 97477_Patient-07ut_uterus | 0.2 | 94711_Donor 2 AM - C_adipose | 6.9 |
| 97478_Patient-07pl_placenta | 0.1 | 94712_Donor 2 AD - A_adipose | 100.0 |
| 99167_Bayer Patient 1 | 4.7 | 94713_Donor 2 AD - B_adipose | 79.6 |
| 97482_Patient-08ut_uterus | 0.1 | 94714_Donor 2 AD - C_adipose | 92.7 |
| 97483_Patient-08pl_placenta | 0.1 | 94742_Donor 3 U - A_Mesenchymal | 0.4 |
| | | Stem Cells | |
| 97486_Patient-09sk_skeletal | 0.4 | 94743_Donor 3 U - B_Mesenchymal | 0.4 |
| muscle | | Stem Cells | |
| 97487_Patient-09ut_uterus | 0.2 | 94730_Donor 3 AM - A_adipose | 8.9 |
| 97488_Patient-09pl_placenta | 0.2 | 94731_Donor 3 AM - B_adipose | 5.4 |
| 97492_Patient-10ut_uterus | 0.3 | 94732_Donor 3 AM - C_adipose | 3.6 |
| 97493_Patient-10pl_placenta | 0.2 | 94733_Donor 3 AD - A_adipose | 66.4 |
| 97495_Patient-11go_adipose | 7.6 | 94734_Donor 3 AD - B_adipose | 21.6 |
| 97496_Patient-11sk_skeletal | 0.7 | 94735_Donor 3 AD - C_adipose | 8.3 |
| muscle | | | |
| 97497_Patient-11ut_uterus | 0.3 | 77138_Liver_HepG2untreated | 14.8 |
| 97498_Patient-11pl_placenta | 0.2 | 73556_Heart_Cardiac stromal cells | 0.1 |
| | | (primary) | |
| 97500_Patient-12go_adipose | 21.0 | 81735_Small Intestine | 1.7 |
| 97501_Patient-12sk_skeletal | 1.8 | 72409_Kidney_Proximal Convoluted | 0.2 |
| muscle | | Tubule | |
| 97502_Patient-12ut_uterus | 0.3 | 82685_Small intestine_Duodenum | 1.5 |
| 97503_Patient-12pl_placenta | 0.7 | 90650_Adrenal_Adrenocortical | 0.0 |
| | | adenoma | |
| 94721_Donor 2 U - | 0.1 | 72410_Kidney_HRCE | 0.6 |
| A_Mesenchymal Stem Cells | | | |
| 94722_Donor 2 U - | 0.4 | 72411_Kidney_HRE | 0.0 |
| B_Mesenchymal Stem Cells | | | |
| 94723_Donor 2 U - | 0.1 | 73139_Uterus_Uterine smooth | 0.0 |
| C_Mesenchymal Stem Cells | | muscle cells | |

General_screening_panel_v1.5 Summary: Ag6056 Highest expression of this gene is detected in colon cancer CaCo-2 cell line (CT=26.3). Moderate to high expression of this gene is also seen in number of cancer cell lines derived from pancreatic, gastric, colon, lung, liver, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, melanoma and brain cancers.

Among tissues with metabolic or endocrine function, this gene is expressed at high to moderate levels in pancreas, adipose, adrenal gland, thyroid, skeletal muscle, heart, liver and the gastrointestinal tract. Therefore, therapeutic modulation of the activity of this gene may prove useful in the treatment of endocrine/metabolically related diseases, such as obesity and diabetes.

This gene codes for Diacylglycerol acyltransferase 2 (DGAT2). DGAT2 catalyzes a reaction in which diacylglycerol is covalently joined to long chain fatty acyl-CoAs. At Curagen using GeneCalling studies expression of DGAT2 was found to be dysregulated in two distinct models of obesity. In a model of genetic obesity DGAT2 expression was increased 2.1 fold in AKR/J (obese) versus C57L/J (normal) mice. DGAT2 expression was also found to be decreased 1.5 fold in a model diet-induced obesity when comparing brown adipose between obese hyperglycemic versus control chow fed mice. These studies indicate that DGAT2 is an excellent molecule for small molecule therapy for the treatment of obesity and prevention of type II diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 5 Islet Summary: Ag6056 Highest expression of this gene is detected in differentiated adipose tissue (CT=26.4). Moderate to high expression of this gene is also seen in adipose, skeletal muscle, small intestine and pancreatic islet cells from diabetic and obese patient. Interestingly, expression of this gene is higher in differentiated adipose compared to undifferentiated and midway differentiated tissue. Thus therapeutic modulation of this gene through the use of small molecule drug may be useful in the treatment of obesity and diabetes, especially type II diabetes.

H. CG148278-01: Human Longchain Acyl CoA Synthetase 1-Like Protein.

Expression of gene CG148278-01 was assessed using the primer-probe sets Ag5215 and Ag5820, described in Tables HA and HB.

TABLE HA

Probe Name Ag5215

| | | | Start | SEQ ID |
|---|---|---|---|---|
| Primers | Sequence | Length | Position | No |
| Forward | 5′-ccagacgactcaccaccttctg-3′ | 22 | 180 | 380 |
| Probe | TET-5′-cggccacgccacccaaaaccc-3′-TAMRA | 21 | 203 | 381 |
| Reverse | 5′-actgcatggagaggtgccat-3′ | 20 | 235 | 382 |

TABLE HR

Probe Name Ag5820

| | | | Start | SEQ ID |
|---|---|---|---|---|
| Primers | Sequence | Length | Position | No |
| Forward | 5′-ctgccttacagtcacctcag-3′ | 22 | 0 | 383 |
| Probe | TET-5′-tgttcagaccatgtttatggtaatacacacttcc-3′-TAMRA | 34 | 2362 | 384 |
| Reverse | 5′-tctcaaataattagcacatttatagtat-3′ | 28 | 2423 | 385 |

I. CG152981-01 and CG152981-02: Corticosteroid 11-Beta Dehydrogenase, Isozyme 1-Like Protein.

Expression of gene CG152981-01 and CG152981-02 was assessed using the primer-probe sets Ag3951 and Ag5951, described in Table IA and IB. Results of the RTQ-PCR runs are shown in Tables IC and ID. Please note that probe-primer set Ag3951 is specific for CG152981-01 and Ag5951 is specific for CG152981-02.

TABLE IA

Probe Name Ag3951

| | | | Start | SEQ ID |
|---|---|---|---|---|
| Primers | Sequence | Length | Position | No |
| Forward | 5′-cttcggagcttttgcagca-3′ | 19 | 108 | 386 |
| Probe | TET-5′-ctcaccaccttctggtacgccacgaga-3′-TAMRA | 27 | 127 | 387 |
| Reverse | 5′-agaggtcgcatggcggctt-3′ | 19 | 166 | 388 |

TABLE TB

Probe Name Ag5951

| | | | Start | SEQ ID |
|---|---|---|---|---|
| Primers | Sequence | Length | Position | No |
| Forward | 5′-aagcagagcaatggaagcatt-3′ | 21 | 557 | 389 |
| Probe | TET-5′-ctctggctgaaacagccatgaaggca-3′-TAMRA | 26 | 591 | 390 |
| Reverse | 5′-ggagctgcttgcatatggact-3′ | 21 | 628 | 391 |

TABLE IC
General_screening_panel_v1.6

| | Rel. |
|---|---|
| | Exp. (%) |
| | Ag3951, |
| | Run |
| Tissue Name | 277231320 |
| Adipose | 38.7 |
| Melanoma* Hs688(A).T | 6.8 |
| Melanoma* Hs688(B).T | 5.9 |
| Melanoma* M14 | 66.4 |
| Melanoma* LOXIMVI | 0.5 |
| Melanoma* SK-MEL-5 | 47.6 |
| Squamous cell carcinoma SCC-4 | 2.1 |
| Testis Pool | 9.0 |
| Prostate ca.* (bone met) PC-3 | 3.1 |
| Prostate Pool | 17.6 |
| Placenta | 2.3 |
| Uterus Pool | 2.3 |
| Ovarian ca. OVCAR-3 | 4.8 |
| Ovarian ca. SK-OV-3 | 12.2 |
| Ovarian ca. OVCAR-4 | 7.5 |
| Ovarian ca. OVCAR-5 | 6.2 |
| Ovarian ca. IGROV-1 | 5.7 |
| Ovarian ca. OVCAR-8 | 0.6 |
| Ovary | 1.9 |
| Breast ca. MCF-7 | 2.1 |
| Breast ca. MDA-MB-231 | 6.8 |
| Breast ca. BT 549 | 8.0 |
| Breast ca. T47D | 5.4 |
| Breast ca. MDA-N | 10.7 |
| Breast Pool | 4.8 |
| Trachea | 12.6 |
| Lung | 0.4 |
| Fetal Lung | 8.9 |
| Lung ca. NCI-N417 | 5.9 |
| Lung ca. LX-1 | 1.4 |
| Lung ca. NCI-H146 | 1.7 |
| Lung ca. SHP-77 | 8.5 |
| Lung ca. A549 | 3.3 |
| Lung ca. NCI-H526 | 2.6 |
| Lung ca. NCI-H23 | 2.7 |
| Lung ca. NCI-H460 | 4.2 |
| Lung ca. HOP-62 | 1.3 |
| Lung ca. NCI-H522 | 2.9 |
| Liver | 85.3 |
| Fetal Liver | 49.3 |
| Liver ca. HepG2 | 0.4 |
| Kidney Pool | 7.6 |
| Fetal Kidney | 4.2 |
| Renal ca. 786-0 | 8.4 |
| Renal ca. A498 | 5.0 |
| Renal ca. ACHN | 50.7 |
| Renal ca. UO-11 | 10.7 |
| Renal ca. TK-10 | 1.6 |
| Bladder | 13.7 |
| Gastric ca. (liver met.) NCI-N87 | 100.0 |
| Gastric ca. KATO III | 48.3 |
| Colon ca. SW-948 | 6.5 |
| Colon ca. SW480 | 17.2 |
| Colon ca.* (SW480 met) SW620 | 2.0 |
| Colon ca. HT29 | 4.4 |
| Colon ca. HCT-116 | 4.5 |
| Colon ca. CaCo-2 | 9.2 |
| Colon cancer tissue | 11.5 |
| Colon ca. SW1116 | 3.6 |
| Colon ca. Colo-205 | 8.4 |
| Colon ca. SW-48 | 4.4 |
| Colon Pool | 5.8 |
| Small Intestine Pool | 3.9 |
| Stomach Pool | 6.2 |
| Bone Marrow Pool | 3.1 |
| Fetal Heart | 4.0 |
| Heart Pool | 12.9 |
| Lymph Node Pool | 7.1 |
| Fetal Skeletal Muscle | 7.8 |
| Skeletal Muscle Pool | 16.8 |
| Spleen Pool | 10.4 |
| Thymus Pool | 9.3 |
| CNS cancer (glio/astro) U87-MG | 17.4 |
| CNS cancer (glio/astro) U-118-MG | 8.7 |
| CNS cancer (neuro; met) SK-N-AS | 3.1 |
| CNS cancer (astro) SF-539 | 4.3 |
| CNS cancer (astro) SNB-75 | 14.8 |
| CNS cancer (glio) SNB-19 | 5.3 |
| CNS cancer (glio) SF-295 | 8.6 |
| Brain (Amygdala) Pool | 7.1 |
| Brain (cerebellum) | 7.8 |
| Brain (fetal) | 5.9 |
| Brain (Hippocampus) Pool | 8.4 |
| Cerebral Cortex Pool | 6.6 |
| Brain (Substantia nigra) Pool | 6.5 |
| Brain (Thalamus) Pool | 10.5 |
| Brain (whole) | 10.4 |
| Spinal Cord Pool | 15.1 |
| Adrenal Gland | 13.9 |
| Pituitary gland Pool | 2.0 |
| Salivary Gland | 12.9 |
| Thyroid (female) | 4.2 |
| Pancreatic ca. CAPAN2 | 1.2 |
| Pancreas Pool | 3.3 |

TABLE ID
Panel 5 Islet

| | Rel. |
|---|---|
| | Exp. (%) |
| | Ag395, |
| | Run |
| Tissue Name | 304686272 |
| 97457_Patient-02go_adipose | 12.9 |
| 97476_Patient-07sk_skeletal muscle | 0.0 |
| 97477_Patient-07ut_uterus | 1.6 |
| 97478_Patient-07pl_placenta | 0.9 |
| 99167_Bayer Patient 1 | 0.0 |
| 97482_Patient-08ut_uterus | 2.1 |
| 97483_Patient-08pl_placenta | 0.7 |
| 97486_Patient-09sk_skeletal muscle | 14.0 |
| 97487_Patient-09ut_uterus | 3.0 |
| 97488_Patient-09pl_placenta | 0.4 |
| 97492_Patient-10ut_uterus | 3.9 |
| 97493_Patient-10pl_placenta | 1.8 |
| 97495_Patient-11go_adipose | 6.7 |
| 97496_Patient-11sk_skeletal muscle | 17.0 |
| 97497_Patient-11ut_uterus | 3.7 |
| 97498_Patient-11pl_placenta | 0.5 |
| 97500_Patient-12go_adipose | 17.4 |
| 97501_Patient-12sk_skeletal muscle | 43.8 |
| 97502_Patient-12ut_uterus | 4.9 |
| 97503_Patient-12pl_placenta | 1.6 |
| 94721_Donor 2 U - A_Mesenchymal Stem Cells | 3.6 |
| 94722_Donor 2 U - B_Mesenchymal Stem Cells | 2.9 |
| 94723_Donor 2 U - C_Mesenchymal Stem Cells | 3.6 |
| 94709_Donor 2 AM - A_adipose | 18.0 |
| 94710_Donor 2 AM - B_adipose | 14.1 |
| 94711_Donor 2 AM - C_adipose | 12.0 |
| 94712_Donor 2 AD - A_adipose | 74.2 |
| 94713_Donor 2 AD - B_adipose | 94.0 |
| 94714_Donor 2 AD - C_adipose | 80.7 |
| 94742_Donor 3 U - A_Mesenchymal Stem Cells | 1.6 |
| 94743_Donor 3 U - B_Mesenchymal Stem Cells | 1.4 |
| 94730_Donor 3 AM - A adipose | 23.2 |
| 94731_Donor 3 AM - B_adipose | 27.5 |
| 94732_Donor 3 AM - C_adipose | 24.3 |
| 94733_Donor 3 AD - A_adipose | 100.0 |
| 94734_Donor 3 AD - B_adipose | 67.8 |
| 94735_Donor 3 AD - C_adipose | 19.8 |
| 77138_Liver_HepG2untreated | 6.2 |
| 73556_Heart_Cardiac stromal cells (primary) | 0.2 |
| 81735 Small Intestine | 5.8 |
| 72409_Kidney_Proximal Convoluted Tubule | 40.3 |
| 82685_Small intestine_Duodenum | 1.6 |
| 90650_Adrenal_Adrenocortical adenoma | 1.7 |
| 72410_Kidney_HRCE | 16.7 |
| 72411_Kidney_HRE | 5.7 |
| 73139_Uterus_Uterine smooth muscle cells | 3.7 |

General_screening_panel_v1.6 Summary: Ag3951 Highest expression of this gene is seen in gastric cancer NCI-N87 cell line (CTs=23.5). High expression of this gene is detected in number of cancer cell lines derived from melanoma, pancreatic, brain, colon, lung, breast, renal, ovarian and prostate cancer. Therefore, therapeutic modulation of this gene may be useful in the treatment of these cancers.

High levels of expression of this gene is also seen in tissues with metabolic/endocrine functions including adipose, and liver. Moderate to low expression are also seen in pancreas, thyroid, adrenal gland, pituitary, smooth muscle, heart and gastrointestinal tract. This gene codes for a variant of long chain acyl-CoA synthetase 2 (LACS2). It is a microsomal enzyme involved in fatty acid esterification. Using CuraGen's GeneCalling™ method of differential gene expression, the rat orthologue of LACS2 was found to be up-regulated in liver in response to troglitazone (TZD) treatment; the mouse orthologue LACS2 was found to be down-regulated in brown adipose tissue, but not in white adipose tissue of obese mice on a high fat diet as compared to chow-fed mice. These data suggest that human LACS2 may contribute to the obese phenotype induced by TZD treatment and may become selectively down-regulated in brown adipose tissue to inhibit fatty acid esterification and promote beta-oxidation. Therefore, an antagonist for LACS2 may be beneficial in the treatment of obesity. In addition, therapeutic modulation of LACS2 encoded by this gene through the use of small molecule drug may be beneficial in the treatment of other metabolic related diseases such as diabetes.

Panel 5 Islet Summary: Ag3951 Highest expression of this gene is detected in differentiated adipose tissue (CT=25.2). This gene shows ubiquitous expression with high expression in adipose tissue. Expression of this gene is higher in differentiated adipose tissues as compared to the mesenchymal stem cells and midway differentiated adipose tissues. Thus, LACS2 protein encoded by this gene may play a role in adipose differentiation. Please see panel 1.6 for further discussion of this gene.

J. CGI59035-01: Glucuronosyltransferase-Like Protein.

Expression of gene CG159035-01 was assessed using the primer-probe set Ag5541, described in Table JA.

TABLE JA

Probe Name Ag5541

| | | | Start | SEQ ID |
|---|---|---|---|---|
| Primers | Sequence | Length | Position | No |
| Forward | 5′-tccacttctggattcaggatt-3′ | 21 | 683 | 392 |
| Probe | TET-5′-aaggcattaggaagacccactacctt-3′-TAMRA | 26 | 736 | 393 |
| Reverse | 5′-gctttccccattgtctcaa-3′ | 19 | 764 | 394 |

K. CG159232-01: Human cAMP-Specific Phosphodiesterase 8 B1-Like Protein.

Expression of gene CG159232-01 was assessed using the primer-probe set Ag5542, described in Table KA. Results of the RTQ-PCR runs are shown in Tables KB, KC and KD.

TABLE KA

Probe Name Ag5542

| | | | Start | SEQ ID |
|---|---|---|---|---|
| Primers | Sequence | Length | Position | No |
| Forward | 5′-agcgtgaagcaggtgtctt-3′ | 19 | 376 | 395 |
| Probe | TET-5′-ccatgagactgacgcaggaccctatt-3′TAMRA | 26 | 416 | 396 |
| Reverse | 5′-ttgcaaagatcagcaaaacct-3′ | 21 | 443 | 397 |

TABLE KB
AI_comprehensive_panel_v1.0

| | Rel. |
|---|---|
| | Exp. (%) |
| | Ag5542, |
| | Run |
| Tissue Name | 245062270 |
| 110967 COPD-F | 38.7 |
| 110980 COPD-F | 11.2 |
| 110968 COPD-M | 25.5 |
| 110977 COPD-M | 18.8 |
| 110989 Emphysema-F | 47.6 |
| 110992 Emphysema-F | 4.9 |
| 110993 Emphysema-F | 36.3 |
| 110994 Emphysema-F | 42.0 |
| 110995 Emphysema-F | 12.3 |
| 110996 Emphysema-F | 0.8 |
| 110997 Asthma-M | 2.9 |
| 111001 Asthma-F | 0.7 |
| 111002 Asthma-F | 51.1 |
| 111003 Atopic Asthma-F | 36.6 |
| 111004 Atopic Asthma-F | 20.6 |
| 111005 Atopic Asthma-F | 21.8 |
| 111006 Atopic Asthma-F | 6.0 |
| 111417 Allergy-M | 22.1 |
| 112347 Allergy-M | 3.2 |
| 112349 Normal Lung-F | 2.5 |
| 112357 Normal Lung-F | 2.4 |
| 112354 Normal Lung-M | 6.4 |
| 112374 Crohns-F | 76.8 |
| 112389 Match Control Crohns-F | 2.9 |
| 112375 Crohns-F | 61.6 |
| 112732 Match Control Crohns-F | 1.0 |
| 112725 Crohns-M | 17.8 |
| 112387 Match Control Crohns-M | 36.9 |
| 112378 Crohns-M | 0.0 |
| 112390 Match Control Crohns-M | 55.5 |
| 112726 Crohns-M | 37.4 |
| 112731 Match Control Crohns-M | 17.4 |
| 112380 Ulcer Col-F | 46.0 |
| 112734 Match Control Ulcer Col-F | 1.9 |
| 112384 Ulcer Col-F | 22.7 |
| 112737 Match Control Ulcer Col-F | 14.6 |
| 112386 Ulcer Col-F | 14.5 |
| 112738 Match Control Ulcer Col-F | 0.9 |
| 112381 Ulcer Col-M | 2.3 |
| 112735 Match Control Ulcer Col-M | 40.3 |
| 112382 Ulcer Col-M | 4.2 |
| 112394 Match Control Ulcer Col-M | 23.3 |
| 112383 Ulcer Col-M | 2.7 |
| 112736 Match Control Ulcer Col-M | 2.6 |
| 112423 Psoriasis-F | 17.6 |
| 112427 Match Control Psoriasis-F | 89.5 |
| 112418 Psoriasis-M | 48.0 |
| 112723 Match Control Psoriasis-M | 0.5 |
| 112419 Psoriasis-M | 55.5 |
| 112424 Match Control Psoriasis-M | 26.6 |
| 112420 Psoriasis-M | 100.0 |
| 112425 Match Control Psoriasis-M | 72.7 |
| 104689 (MF) OA Bone-Backus | 5.6 |
| 104690 (MF) Adj “Normal” Bone-Backus | 14.0 |
| 104691 (MF) OA Synovium-Backus | 3.3 |
| 104692 (BA) OA Cartilage-Backus | 0.0 |
| 104694 (BA) OA Bone-Backus | 1.7 |
| 104695 (BA) Adj “Normal” Bone-Backus | 11.2 |
| 104696 (BA) OA Synovium-Backus | 1.2 |
| 104700 (SS) OA Bone-Backus | 1.7 |
| 104701 (SS) Adj “Normal” Bone-Backus | 4.4 |
| 104702 (SS) OA Synovium-Backus | 12.4 |
| 117093 OA Cartilage Rep7 | 24.3 |
| 112672 OA Bone5 | 15.6 |
| 112673 OA Synovium5 | 8.1 |
| 112674 OA Synovial Fluid cellsS | 8.1 |
| 117100 OA Cartilage Rep14 | 9.9 |
| 112756 OA Bone9 | 1.2 |
| 112757 OA Synovium9 | 0.0 |
| 112758 OA Synovial Fluid Cells9 | 18.6 |
| 117125 RA Cartilage Rep2 | 45.1 |
| 113492 Bone2 RA | 5.3 |
| 113493 Synovium2 RA | 0.3 |
| 113494 Syn Fluid Cells RA | 3.7 |
| 113499 Cartilage4 RA | 0.7 |
| 113500 Bone4 RA | 3.5 |
| 113501 Synovium4 RA | 3.0 |
| 113502 Syn Fluid Cells4 RA | 2.4 |
| 113495 Cartilage3 RA | 2.0 |
| 113496 Bone3 RA | 1.8 |
| 113497 Synovium3 RA | 1.9 |
| 113498 Syn Fluid Cells3 RA | 2.8 |
| 117106 Normal Cartilage Rep20 | 8.7 |
| 113663 Bone3 Normal | 4.3 |
| 113664 Synovium3 Normal | 1.2 |
| 113665 Syn Fluid Cells3 Normal | 2.0 |
| 117107 Normal Cartilage Rep22 | 12.2 |
| 113667 Bone4 Normal | 26.6 |
| 113668 Synovium4 Normal | 23.3 |
| 113669 Syn Fluid Cells4 Normal | 21.0 |

TABLE KC
Panel 5 Islet

| | Rel. |
|---|---|
| | Exp. (%) |
| | Ag5542, |
| | Run |
| Tissue Name | 277224359 |
| 97457_Patient-02go_adipose | 40.3 |
| 97476_Patient-07sk_skeletal muscle | 1.9 |
| 97477_Patient-07ut_uterus | 19.6 |
| 97478_Patient-07pl_placenta | 18.8 |
| 99167_Bayer Patient 1 | 2.7 |
| 97482_Patient-08ut_uterus | 31.6 |
| 97483_Patient-08pl_placenta | 18.0 |
| 97486_Patient-09sk_skeletal muscle | 0.0 |
| 97487_Patient-09ut_uterus | 100.0 |
| 97488_Patient-09pl_placenta | 8.4 |
| 97492_Patient-10ut_uterus | 18.7 |
| 97493_Patient-10pl_placenta | 8.2 |
| 97495_Patient-11go_adipose | 30.6 |
| 97496_Patient-11sk_skeletal muscle | 0.0 |
| 97497_Patient-11ut_uterus | 92.7 |
| 97498_Patient-11pl_placenta | 9.5 |
| 97500_Patient-12go_adipose | 20.6 |
| 97501_Patient-12sk_skeletal muscle | 0.0 |
| 97502_Patient-12ut_uterus | 68.8 |
| 97503_Patient-12pl_placenta | 6.6 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.3 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.9 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.9 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 13.6 |
| 72409_Kidney_Proximal Convoluted Tubule | 0.0 |
| 82685_Small intestine Duodenum | 0.8 |
| 90650_Adrenal_Adrenocortical adenoma | 2.2 |
| 72410_Kidney_HRCE | 0.0 |
| 72411_Kidney_HRE | 0.0 |
| 73139_Uterus_Uterine smooth muscle cells | 0.0 |

TABLE KD
general_oncology_screening_panel_v_2.4

| | Rel. Exp. (%) |
|---|---|
| | Ag5542, |
| | Run |
| Tissue Name | 260268947 |
| Colon cancer 1 | 4.5 |
| Colon NAT 1 | 4.0 |
| Colon cancer 2 | 0.6 |
| Colon NAT 2 | 2.3 |
| Colon cancer 3 | 3.8 |
| Colon NAT 3 | 6.6 |
| Colon malignant cancer 4 | 0.0 |
| Colon NAT 4 | 1.5 |
| Lung cancer 1 | 0.4 |
| Lung NAT 1 | 2.3 |
| Lung cancer 2 | 6.0 |
| Lung NAT 2 | 7.6 |
| Squamous cell carcinoma 3 | 2.3 |
| Lung NAT 3 | 1.0 |
| Metastatic melanoma 1 | 30.8 |
| Melanoma 2 | 0.5 |
| Melanoma 3 | 0.3 |
| Metastatic melanoma 4 | 11.9 |
| Metastatic melanoma 5 | 15.8 |
| Bladder cancer 1 | 0.3 |
| Bladder NAT 1 | 0.0 |
| Bladder cancer 2 | 2.0 |
| Bladder NAT 2 | 0.0 |
| Bladder NAT 3 | 0.6 |
| Bladder NAT 4 | 1.7 |
| Prostate adenocarcinoma 1 | 100.0 |
| Prostate adenocarcinoma 2 | 9.2 |
| Prostate adenocarcinoma 3 | 81.2 |
| Prostate adenocarcinoma 4 | 8.6 |
| Prostate NAT 5 | 11.7 |
| Prostate adenocarcinoma 6 | 28.9 |
| Prostate adenocarcinoma 7 | 24.7 |
| Prostate adenocarcinoma 8 | 7.4 |
| Prostate adenocarcinoma 9 | 77.4 |
| Prostate NAT 10 | 8.5 |
| Kidney cancer 1 | 1.5 |
| Kidney NAT 1 | 3.9 |
| Kidney cancer 2 | 12.4 |
| Kidney NAT 2 | 52.9 |
| Kidney cancer 3 | 1.4 |
| Kidney NAT 3 | 7.5 |
| Kidney cancer 4 | 0.6 |
| Kidney NAT 4 | 3.4 |

AI_comprehensive panel_v1.0 Summary: Ag5542 Highest expression of this gene is detected in psoriasis sample (CT=30). Moderate to low expression of this gene is also seen in samples derived from normal and orthoarthitis arthritis bone, cartilage, synovium and synovial fluid samples, from COPD lung, emphysema, atopic asthma, asthma, allergy, Crohn's disease (normal matched control and diseased), ulcerative colitis (normal matched control and diseased), and psoriasis (normal matched control and diseased). Therefore, therapeutic modulation of this gene product may ameliorate symptoms/conditions associated with autoimmune and inflammatory disorders including psoriasis, allergy, asthma, inflammatory bowel disease including Crohns and ulcerative colitis and osteoarthritis.

Panel 5 Islet Summary: Ag5542 Highest expression of this gene is detected in uterus of non-diabetic but obese patient (CT=31.6). Moderate to low expression of this gene is detected in adipose, uterus, and small intestine. Therefore, therapeutic modulation of this gene may be useful in the treatment of metabolic/endocrine diseases including diabetes and obesity.

general oncology screening panel_v_{—}2.4 Summary: Ag5542 Highest expression of this gene is detected in prostate adenocarcinoma sample (CT=30.3). Moderate to low expression of this gene is also seen in metastatic melanoma, normal and cancer sample from lung and kidney. Interestingly, expression of this gene is higher in metastatic melanoma and prostate cancer. Therefore, expression of this gene can used as diagnostic marker to detect the presence of metastic melanoma and prostate cancer. In addition, therapeutic modulation of this gene may be useful in the treatment of metastatic melanoma, prostate cancer, lung and kidney cancers.

L. CG160563-01: Monocarboxylate Transporter 7-Like Protein.

Expression of gene CG160563-01 was assessed using the primer-probe set Ag3575, described in Table LA. Results of the RTQ-PCR runs are shown in Tables LB, LC, LD, LE, LF and LG. Please note that CG160563-01 represents a full length physical clone.

TABLE LA

Probe Name Ag3575

| | | | | |
|---|---|---|---|---|
| | | | | SEQ ID |
| Primers | Sequence | Length | Start Position | No |

| | | | | |
|---|---|---|---|---|
| Forward | 5′-tgctttagttttctcccaactg-3′ | 22 | 1209 | 398 |
| Probe | TET-5′-ccatcctatcacaatattttggcaaa-3′-TAMRA | 26 | 1180 | 399 |
| Reverse | 5′-aactgcagtgactatggaacgt-3′ | 22 | 1156 | 400 |

TABLE LB
CNS_neurodegeneration_v1.0

| | Rel. |
|---|---|
| | Exp. (%) |
| | Ag3575, |
| | Run |
| Tissue Name | 210629744 |
| AD 1 Hippo | 3.8 |
| AD 2 Hippo | 12.0 |
| AD 3 Hippo | 2.8 |
| AD 4 Hippo | 2.2 |
| AD 5 hippo | 82.4 |
| AD 6 Hippo | 32.3 |
| Control 2 Hippo | 20.6 |
| Control 4 Hippo | 74.7 |
| Control (Path) 3 Hippo | 90.1 |
| AD 1 Temporal Ctx | 2.6 |
| AD 2 Temporal Ctx | 19.1 |
| AD 3 Temporal Ctx | 1.4 |
| AD 4 Temporal Ctx | 9.0 |
| AD 5 Inf Temporal Ctx | 41.2 |
| AD 5 Sup Temporal Ctx | 100.0 |
| AD 6 Inf Temporal Ctx | 50.3 |
| AD 6 Sup Temporal Ctx | 49.3 |
| Control 1 Temporal Ctx | 6.9 |
| Control 2 Temporal Ctx | 25.9 |
| Control 3 Temporal Ctx | 12.8 |
| Control 4 Temporal Ctx | 5.8 |
| Control (Path) 1 Temporal Ctx | 42.9 |
| Control (Path) 2 Temporal Ctx | 32.1 |
| Control (Path) 3 Temporal Ctx | 5.8 |
| Control (Path) 4 Temporal Ctx | 27.2 |
| AD 1 Occipital Ctx | 6.0 |
| AD 2 Occipital Ctx (Missing) | 0.0 |
| AD 3 Occipital Ctx | 2.4 |
| AD 4 Occipital Ctx | 15.1 |
| AD 5 Occipital Ctx | 28.3 |
| AD 6 Occipital Ctx | 54.7 |
| Control 1 Occipital Ctx | 9.4 |
| Control 2 Occipital Ctx | 72.2 |
| Control 3 Occipital Ctx | 23.8 |
| Control 4 Occipital Ctx | 12.4 |
| Control (Path) 1 Occipital Ctx | 65.5 |
| Control (Path) 2 Occipital Ctx | 11.7 |
| Control (Path) 3 Occipital Ctx | 5.9 |
| Control (Path) 4 Occipital Ctx | 17.8 |
| Control 1 Parietal Ctx | 6.8 |
| Control 2 Parietal Ctx | 13.4 |
| Control 3 Parietal Ctx | 33.4 |
| Control (Path) 1 Parietal Ctx | 76.8 |
| Control (Path) 2 Parietal Ctx | 25.3 |
| Control (Path) 3 Parietal Ctx | 9.8 |
| Control (Path) 4 Parietal Ctx | 42.9 |

TABLE LC
General_screening_panel_v1.4

| | Rel. |
|---|---|
| | Exp. (%) |
| | Ag3575, |
| | Run |
| Tissue Name | 217343280 |
| Adipose | 0.1 |
| Melanoma* Hs688(A).T | 0.0 |
| Melanoma* Hs688(B).T | 0.0 |
| Melanoma* M14 | 17.4 |
| Melanoma* LOXIMVI | 0.0 |
| Melanoma* SK-MEL-5 | 100.0 |
| Squamous cell carcinoma SCC-4 | 0.1 |
| Testis Pool | 0.8 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.4 |
| Ovarian ca. SK-OV-3 | 0.0 |
| Ovarian ca. OVCAR-4 | 0.0 |
| Ovarian ca. OVCAR-5 | 0.5 |
| Ovarian ca. IGROV-1 | 0.0 |
| Ovarian ca. OVCAR-8 | 0.0 |
| Ovary | 0.1 |
| Breast ca. MCF-7 | 3.0 |
| Breast ca. MDA-MB-231 | 0.0 |
| Breast ca. BT 549 | 0.3 |
| Breast ca. T47D | 1.2 |
| Breast ca. MDA-N | 0.0 |
| Breast Pool | 0.1 |
| Trachea | 0.2 |
| Lung | 0.0 |
| Fetal Lung | 0.2 |
| Lung ca. NCI-N417 | 0.1 |
| Lung ca. LX-1 | 2.7 |
| Lung ca. NCI-H146 | 0.1 |
| Lung ca. SHP-77 | 0.0 |
| Lung ca. A549 | 0.0 |
| Lung ca. NCI-H526 | 0.1 |
| Lung ca. NCI-H23 | 0.2 |
| Lung ca. NCI-H460 | 6.3 |
| Lung ca. HOP-62 | 0.0 |
| Lung ca. NCI-H522 | 0.6 |
| Liver | 0.0 |
| Fetal Liver | 0.1 |
| Liver ca. HepG2 | 0.5 |
| Kidney Pool | 0.1 |
| Fetal Kidney | 0.1 |
| Renal ca. 786-0 | 0.1 |
| Renal ca. A498 | 0.0 |
| Renal ca. ACHN | 0.1 |
| Renal ca. UO-31 | 0.3 |
| Renal ca. TK-10 | 0.4 |
| Bladder | 0.2 |
| Gastric ca. (liver met.) NCI-N87 | 1.1 |
| Gastric ca. KATO III | 0.0 |
| Colon ca. SW-948 | 0.0 |
| Colon ca. SW480 | 1.8 |
| Colon ca.* (SW480 met) SW620 | 2.6 |
| Colon ca. HT29 | 0.0 |
| Colon ca. HCT-116 | 0.7 |
| Colon ca. CaCo-2 | 0.1 |
| Colon cancer tissue | 0.2 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.1 |
| Stomach Pool | 0.1 |
| Bone Marrow Pool | 0.1 |
| Fetal Heart | 0.0 |
| Heart Pool | 0.0 |
| Lymph Node Pool | 0.1 |
| Fetal Skeletal Muscle | 0.0 |
| Skeletal Muscle Pool | 0.2 |
| Spleen Pool | 0.3 |
| Thymus Pool | 0.2 |
| CNS cancer (glio/astro) U87-MG | 2.5 |
| CNS cancer (glio/astro) U-118-MG | 1.3 |
| CNS cancer (neuro; met) SK-N-AS | 0.0 |
| CNS cancer (astro) SF-539 | 0.0 |
| CNS cancer (astro) SNB-75 | 0.1 |
| CNS cancer (glio) SNB-19 | 0.0 |
| CNS cancer (glio) SF-295 | 0.1 |
| Brain (Amygdala) Pool | 0.1 |
| Brain (cerebellum) | 0.2 |
| Brain (fetal) | 0.2 |
| Brain (Hippocampus) Pool | 0.2 |
| Cerebral Cortex Pool | 0.3 |
| Brain (Substantia nigra) Pool | 0.2 |
| Brain (Thalamus) Pool | 0.4 |
| Brain (whole) | 0.2 |
| Spinal Cord Pool | 0.1 |
| Adrenal Gland | 0.7 |
| Pituitary gland Pool | 0.0 |
| Salivary Gland | 0.0 |
| Thyroid (female) | 0.1 |
| Pancreatic ca. CAPAN2 | 0.1 |
| Pancreas Pool | 0.2 |

TABLE LD
General_screening_panel_v1.6

| | Rel. |
|---|---|
| | Exp. (%) |
| | Ag3575, |
| | Run |
| Tissue Name | 277230936 |
| Adipose | 0.1 |
| Melanoma* Hs688(A).T | 0.0 |
| Melanoma* Hs688(B).T | 0.0 |
| Melanoma* M14 | 14.7 |
| Melanoma* LOXIMVI | 0.0 |
| Melanoma* SK-MEL-5 | 100.0 |
| Squamous cell carcinoma SCC-4 | 0.1 |
| Testis Pool | 0.9 |
| Prostate ca.* (bone met) PC-3 | 0.0 |
| Prostate Pool | 0.0 |
| Placenta | 0.0 |
| Uterus Pool | 0.1 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.6 |
| Ovarian ca. IGROV-1 | 0.0 |
| Ovarian ca. OVCAR-8 | 0.0 |
| Ovary | 0.1 |
| Breast ca. MCF-7 | 2.2 |
| Breast ca. MDA-MB-231 | 0.1 |
| Breast ca. BT 549 | 0.2 |
| Breast ca. T47D | 0.7 |
| Breast ca. MDA-N | 0.0 |
| Breast Pool | 0.1 |
| Trachea | 0.1 |
| Lung | 0.0 |
| Fetal Lung | 0.1 |
| Lung ca. NCI-N417 | 0.1 |
| Lung ca. LX-1 | 2.0 |
| Lung ca. NCI-H146 | 0.1 |
| Lung ca. SHP-77 | 0.0 |
| Lung ca. A549 | 0.2 |
| Lung ca. NCI-H526 | 0.1 |
| Lung ca. NCI-H23 | 0.2 |
| Lung ca. NCI-H460 | 6.0 |
| Lung ca. HOP-62 | 0.0 |
| Lung ca. NCI-H522 | 0.4 |
| Liver | 0.0 |
| Fetal Liver | 0.1 |
| Liver ca. HepG2 | 0.4 |
| Kidney Pool | 0.1 |
| Fetal Kidney | 0.1 |
| Renal ca. 786-0 | 0.1 |
| Renal ca. A498 | 0.0 |
| Renal ca. ACHN | 0.1 |
| Renal ca. UO-31 | 0.2 |
| Renal ca. TK-10 | 0.3 |
| Bladder | 0.2 |
| Gastric ca. (liver met.) NCI-N87 | 0.8 |
| Gastric ca. KATO III | 0.0 |
| Colon ca. SW-948 | 0.0 |
| Colon ca. SW480 | 1.5 |
| Colon ca.* (SW480 met) SW620 | 1.4 |
| Colon ca. HT29 | 0.0 |
| Colon ca. HCT-116 | 0.6 |
| Colon ca. CaCo-2 | 0.1 |
| Colon cancer tissue | 0.1 |
| Colon ca. SW1116 | 0.0 |
| Colon ca. Colo-205 | 0.0 |
| Colon ca. SW-48 | 0.0 |
| Colon Pool | 0.1 |
| Small Intestine Pool | 0.1 |
| Stomach Pool | 0.0 |
| Bone Marrow Pool | 0.1 |
| Fetal Heart | 0.0 |
| Heart Pool | 0.0 |
| Lymph Node Pool | 0.1 |
| Fetal Skeletal Muscle | 0.0 |
| Skeletal Muscle Pool | 0.1 |
| Spleen Pool | 0.2 |
| Thymus Pool | 0.2 |
| CNS cancer (glio/astro) U87-MG | 2.2 |
| CNS cancer (glio/astro) U-118-MG | 1.0 |
| CNS cancer (neuro; met) SK-N-AS | 0.0 |
| CNS cancer (astro) SF-539 | 0.0 |
| CNS cancer (astro) SNB-75 | 0.1 |
| CNS cancer (glio) SNB-19 | 0.0 |
| CNS cancer (glio) SF-295 | 0.1 |
| Brain (Amygdala) Pool | 0.1 |
| Brain (cerebellum) | 0.4 |
| Brain (fetal) | 0.2 |
| Brain (Hippocampus) Pool | 0.2 |
| Cerebral Cortex Pool | 0.3 |
| Brain (Substantia nigra) Pool | 0.2 |
| Brain (Thalamus) Pool | 0.3 |
| Brain (whole) | 0.2 |
| Spinal Cord Pool | 0.1 |
| Adrenal Gland | 0.6 |
| Pituitary gland Pool | 0.1 |
| Salivary Gland | 0.0 |
| Thyroid (female) | 0.1 |
| Pancreatic ca. CAPAN2 | 0.1 |
| Pancreas Pool | 0.0 |

TABLE LE
General_screening_panel_v1.7

| | Rel. |
|---|---|
| | Exp. (%) |
| | Ag3575, |
| | Run |
| Tissue Name | 318345840 |
| Adipose | 0.8 |
| HUVEC | 0.3 |
| Melanoma* Hs688(A).T | 0.0 |
| Melanoma* Hs688(B).T | 0.1 |
| Melanoma (met) SK-MEL-5 | 100.0 |
| Testis | 2.0 |
| Prostate ca. (bone met) PC-3 | 0.0 |
| Prostate ca. DU145 | 0.1 |
| Prostate pool | 0.1 |
| Uterus pool | 0.0 |
| Ovarian ca. OVCAR-3 | 0.1 |
| Ovarian ca. (ascites) SK-OV-3 | 0.0 |
| Ovarian ca. OVCAR-4 | 0.4 |
| Ovarian ca. OVCAR-5 | 1.1 |
| Ovarian ca. IGROV-1 | 0.2 |
| Ovarian ca. OVCAR-8 | 0.3 |
| Ovary | 0.2 |
| Breast ca. MCF-7 | 3.8 |
| Breast ca. MDA-MB-231 | 0.1 |
| Breast ca. BT-549 | 0.3 |
| Breast ca. T47D | 3.3 |
| Breast pool | 0.1 |
| Trachea | 0.8 |
| Lung | 1.9 |
| Fetal Lung | 0.5 |
| Lung ca. NCI-N417 | 0.3 |
| Lung ca. LX-1 | 0.7 |
| Lung ca. NCI-H146 | 0.6 |
| Lung ca. SHP-77 | 0.1 |
| Lung ca. NCI-H23 | 0.6 |
| Lung ca. NCI-H460 | 2.3 |
| Lung ca. HOP-62 | 0.3 |
| Lung ca. NCI-H522 | 0.3 |
| Lung ca. DMS-114 | 0.1 |
| Liver | 0.0 |
| Fetal Liver | 0.2 |
| Kidney pool | 0.3 |
| Fetal Kidney | 0.0 |
| Renal ca. 786-0 | 0.0 |
| Renal ca. A498 | 0.0 |
| Renal ca. ACHN | 0.3 |
| Renal ca. UO-31 | 0.7 |
| Renal ca. TK-10 | 0.3 |
| Bladder | 0.1 |
| Gastric ca. (liver met.) NCI-N87 | 0.1 |
| Stomach | 0.0 |
| Colon ca. SW-948 | 0.0 |
| Colon ca. SW480 | 0.1 |
| Colon ca. (SW480 met) SW620 | 9.5 |
| Colon ca. HT29 | 0.8 |
| Colon ca. HCT-116 | 1.9 |
| Colon cancer tissue | 0.0 |
| Colon ca. SW1116 | 0.1 |
| Colon ca. Colo-205 | 0.0 |
| Colon ca. SW-48 | 0.0 |
| Colon | 0.1 |
| Small Intestine | 0.1 |
| Fetal Heart | 0.2 |
| Heart | 0.0 |
| Lymph Node pool 1 | 0.0 |
| Lymph Node pool 2 | 2.0 |
| Fetal Skeletal Muscle | 0.0 |
| Skeletal Muscle pool | 0.1 |
| Skeletal Muscle | 0.2 |
| Spleen | 0.7 |
| Thymus | 0.3 |
| CNS cancer (glio/astro) SF-268 | 0.0 |
| CNS cancer (glio/astro) T98G | 0.8 |
| CNS cancer (neuro; met) SK-N-AS | 0.0 |
| CNS cancer (astro) SF-539 | 0.2 |
| CNS cancer (astro) SNB-75 | 0.2 |
| CNS cancer (glio) SNB-19 | 0.0 |
| CNS cancer (glio) SF-295 | 0.1 |
| Brain (Amygdala) | 2.0 |
| Brain (Cerebellum) | 1.0 |
| Brain (Fetal) | 0.7 |
| Brain (Hippocampus) | 1.4 |
| Cerebral Cortex pool | 0.7 |
| Brain (Substantia nigra) | 0.1 |
| Brain (Thalamus) | 0.9 |
| Brain (Whole) | 3.2 |
| Spinal Cord | 0.2 |
| Adrenal Gland | 3.8 |
| Pituitary Gland | 0.4 |
| Salivary Gland | 0.1 |
| Thyroid | 1.1 |
| Pancreatic ca. PANC-1 | 0.0 |
| Pancreas pool | 0.0 |

TABLE LF
Panel 4.1D

| | Rel. |
|---|---|
| | Exp. (%) |
| | Ag3575, |
| | Run |
| Tissue Name | 169851846 |
| Secondary Th1 act | 3.1 |
| Secondary Th2 act | 7.3 |
| Secondary Tr1 act | 9.3 |
| Secondary Th1 rest | 1.4 |
| Secondary Th2 rest | 5.0 |
| Secondary Tr1 rest | 2.5 |
| Primary Th1 act | 2.9 |
| Primary Th2 act | 3.0 |
| Primary Tr1 act | 3.0 |
| Primary Th1 rest | 1.5 |
| Primary Th2 rest | 2.5 |
| Primary Tr1 rest | 2.5 |
| CD45RA CD4 lymphocyte act | 3.8 |
| CD45RO CD4 lymphocyte act | 9.0 |
| CD8 lymphocyte act | 6.3 |
| Secondary CD8 lymphocyte rest | 6.3 |
| Secondary CD8 lymphocyte act | 6.2 |
| CD4 lymphocyte none | 2.5 |
| 2ry Th1/Th2/Tr1_anti-CD95 CH11 | 4.0 |
| LAK cells rest | 20.7 |
| LAK cells IL-2 | 11.1 |
| LAK cells IL-2 + IL-12 | 9.9 |
| LAK cells IL-2 + IFN gamma | 9.7 |
| LAK cells IL-2 + IL-18 | 8.7 |
| LAK cells PMA/ionomycin | 69.3 |
| NK Cells IL-2 rest | 16.7 |
| Two Way MLR 3 day | 12.3 |
| Two Way MLR 5 day | 6.0 |
| Two Way MLR 7 day | 2.6 |
| PBMC rest | 7.7 |
| PBMC PWM | 3.8 |
| PBMC PHA-L | 3.7 |
| Ramos (B cell) none | 11.2 |
| Ramos (B cell) ionomycin | 7.3 |
| B lymphocytes PWM | 3.9 |
| B lymphocytes CD40L and IL-4 | 5.4 |
| EOL-1 dbcAMP | 9.7 |
| EOL-1 dbcAMP PMA/ionomycin | 26.1 |
| Dendritic cells none | 28.1 |
| Dendritic cells LPS | 8.6 |
| Dendritic cells anti-CD40 | 22.2 |
| Monocytes rest | 26.6 |
| Monocytes LPS | 9.9 |
| Macrophages rest | 11.7 |
| Macrophages LPS | 8.3 |
| HUVEC none | 0.7 |
| HUVEC starved | 0.2 |
| HUVEC IL-1beta | 1.2 |
| HUVEC IFN gamma | 1.3 |
| HUVEC TNF alpha + IFN gamma | 0.0 |
| HUVEC TNF alpha + IL4 | 0.5 |
| HUVEC IL-11 | 0.8 |
| Lung Microvascular EC none | 2.2 |
| Lung Microvascular EC TNFalpha + IL-1beta | 1.2 |
| Microvascular Dermal EC none | 0.6 |
| Microsvasular Dermal EC TNFalpha + IL-1beta | 0.6 |
| Bronchial epithelium TNFalpha + IL1beta | 0.3 |
| Small airway epithelium none | 6.5 |
| Small airway epithelium TNFalpha + IL-1beta | 0.7 |
| Coronery artery SMC rest | 0.3 |
| Coronery artery SMC TNFalpha + IL-1beta | 0.8 |
| Astrocytes rest | 0.7 |
| Astrocytes TNFalpha + IL-1beta | 1.0 |
| KU-812 (Basophil) rest | 0.3 |
| KU-812 (Basophil) PMA/ionomycin | 0.5 |
| CCD1106 (Keratinocytes) none | 0.8 |
| CCD1106 (Keratinocytes) TNFalpha + IL-1beta | 1.1 |
| Liver cirrhosis | 6.2 |
| NCI-H292 none | 1.3 |
| NCI-H292 IL-4 | 17.6 |
| NCI-H292 IL-9 | 4.7 |
| NCI-H292 IL-13 | 13.1 |
| NCI-H292 IFN gamma | 6.1 |
| HPAEC none | 1.0 |
| HPAEC TNF alpha + IL-1 beta | 1.7 |
| Lung fibroblast none | 20.6 |
| Lung fibroblast TNF alpha + IL-1 beta | 100.0 |
| Lung fibroblast IL-4 | 39.0 |
| Lung fibroblast IL-9 | 89.5 |
| Lung fibroblast IL-13 | 40.6 |
| Lung fibroblast IFN gamma | 60.7 |
| Dermal fibroblast CCD1070 rest | 2.3 |
| Dermal fibroblast CCD1070 TNF alpha | 10.7 |
| Dermal fibroblast CCD1070 IL-1 beta | 4.5 |
| Dermal fibroblast IFN gamma | 2.5 |
| Dermal fibroblast IL-4 | 3.1 |
| Dermal Fibroblasts rest | 1.4 |
| Neutrophils TNFa + LPS | 6.2 |
| Neutrophils rest | 17.3 |
| Colon | 0.7 |
| Lung | 6.2 |
| Thymus | 4.7 |
| Kidney | 1.5 |

TABLE LG
Panel 5 Islet

| | Rel. |
|---|---|
| | Exp. (%) |
| | Ag357, |
| | Run |
| Tissue Name | 279370904 |
| 97457_Patient-02go_adipose | 4.4 |
| 97476_Patient-07sk_skeletal muscle | 0.0 |
| 97477_Patient-07ut_uterus | 6.6 |
| 97478_Patient-07pl_placenta | 16.0 |
| 99167_Bayer Patient 1 | 45.1 |
| 97482_Patient-08ut_uterus | 6.5 |
| 97483_Patient-08pl_placenta | 6.8 |
| 97486_Patient-09sk_skeletal muscle | 3.3 |
| 97487_Patient-09ut_uterus | 5.9 |
| 97488_Patient-09pl_placenta | 12.9 |
| 97492_Patient-10ut_uterus | 5.3 |
| 97493_Patient-10pl_placenta | 27.4 |
| 97495_Patient-11go_adipose | 5.3 |
| 97496_Patient-11sk_skeletal muscle | 3.2 |
| 97497_Patient-11ut_uterus | 12.7 |
| 97498_Patient-11pl_placenta | 13.2 |
| 97500_Patient-12go_adipose | 6.3 |
| 97501_Patient-12sk_skeletal muscle | 6.1 |
| 97502_Patient-12ut_uterus | 7.5 |
| 97503_Patient-12pl_placenta | 33.9 |
| 94721_Donor 2 U - A_Mesenchymal Stem Cells | 84.7 |
| 94722_Donor 2 U - B_Mesenchymal Stem Cells | 72.2 |
| 94723_Donor 2 U - C_Mesenchymal Stem Cells | 81.8 |
| 94709_Donor 2 AM - A_adipose | 49.0 |
| 94710_Donor 2 AM - B_adipose | 38.4 |
| 94711_Donor 2 AM - C_adipose | 25.7 |
| 94712_Donor 2 AD - A_adipose | 58.2 |
| 94713_Donor 2 AD - B_adipose | 86.5 |
| 94714_Donor 2 AD - C_adipose | 54.0 |
| 94742_Donor 3 U - A_Mesenchymal Stem Cells | 21.9 |
| 94743_Donor 3 U - B_Mesenchymal Stem Cells | 26.8 |
| 94730_Donor 3 AM - A_adipose | 44.1 |
| 94731_Donor 3 AM - B_adipose | 67.8 |
| 94732_Donor 3 AM - C_adipose | 61.1 |
| 94733_Donor 3 AD - A_adipose | 100.0 |
| 94734_Donor 3 AD - B_adipose | 92.7 |
| 94735_Donor 3 AD - C_adipose | 34.4 |
| 77138_Liver_HepG2untreated | 51.8 |
| 73556_Heart_Cardiac stromal cells (primary) | 5.9 |
| 81735_Small Intestine | 9.2 |
| 72409_Kidney_Proximal Convoluted Tubule | 12.3 |
| 82685_Small_intestine_Duodenum | 13.6 |
| 90650_Adrenal_Adrenocortical adenoma | 6.0 |
| 72410_Kidney_HRCE | 17.7 |
| 72411_Kidney_HRE | 9.9 |
| 73139_Uterus_Uterine smooth muscle cells | 33.0 |

CNS_neurodegeneration_v1.0 Summary: Ag3575 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of this gene in treatment of central nervous system disorders.

General_screening_panel_v1.4 Summary: Ag3575 Highest expression of this gene is detected in melanoma SK-MEL-5 cell line (CT=24.3). Therefore, expression of this gene may be used to distinguish this cell line from other samples in this panel. In addition, expression of this gene can be used as marker for melanoma.

Moderate to low expression of this gene is also seen in number of cancer cell line derived from melanoma, ovarian, breast, lung, renal, gastric, colon and pancreatic cancer. Therefore, therapeutic modulation of this gene or its protein product may be useful in the treatment of melanoma, ovarian, breast, lung, renal, gastric, colon and pancreatic cancers.

Among tissues with metabolic or endocrine function, this gene is expressed at low levels in pancreas, adipose, adrenal gland, thyroid, skeletal muscle, and the gastrointestinal tract. Therefore, therapeutic modulation of the activity of this gene may prove useful in the treatment of endocrine/metabolically related diseases, such as obesity and diabetes.

In addition, this gene is expressed at low levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.6 Summary: Ag3575 Highest expression of this gene is detected in melanoma SK-MEL-5 cell line (CT=23.7). The expression profile in this panel correlates with that of panel 1.4. Please see panel 1.4 for further discussion on the utility of this gene.

General_screening_panel_v1.7 Summary: Ag3575 Highest expression of this gene is detected in melanoma SK-MEL-5 cell line (CT=23.9). The expression profile in this panel correlates with that of panel 1.4. Please see panel 1.4 for further discussion on the utility of this gene.

Panel 4.1D Summary: Ag3575 Highest expression of this gene is detected in TNF alpha+IL-1 beta activated lung fibroblasts (CT=28). Expression of this gene is higher in cytokine activated compared to resting lung fibroblasts and other samples used in this panel. Therefore, therapeutic modulation of this gene or its protein product may be useful in the treatment of pathological and inflammatory lung disorders that include chronic obstructive pulmonary disease, asthma, allergy and emphysema.

This gene is also expressed at moderate to low levels in cell types of significance in the immune response in health and disease. These cells include members of the T-cell, B-cell, macrophage/monocyte, and peripheral blood mononuclear cell family, as well as epithelial and fibroblast cell types from lung and skin, and normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag3575 Highest expression of this gene is detected in differentiated adipose tissue (CT=25.2). High expression of this gene is seen in undifferentiated, midway differentiated and differentiated adipose. Moderate expression of this gene is also seen in other tissues with metabolic/endocrine functions including pancreatic islet cells, adipose, skeletal muscle, small intestine, uterus, placenta, heart and kidney. Please see panel 1.4 for further discussion on the utility of this gene.

M. CG161527-01: Sodium/Potassium-Transporting ATPase Alpha-4 Chain-Like Protein.

Expression of gene CG161527-01 was assessed using the primer-probe set Ag5740, described in Table MA.

TABLE MA

Probe Name Ag5740

| | | | | |
|---|---|---|---|---|
| | | | | SEQ ID |
| Primers | Sequenes | Length | Start Position | No |

| | | | | |
|---|---|---|---|---|
| Forward | 5′-ctctgctttgtggcctacag-3′ | 20 | 829 | 401 |
| Probe | TET-5′-tccagatatatttcaatgaggagcctacca-3′-TAMRA | 30 | 851 | 402 |
| Reverse | 5′-cgatgctcaggtagaggttgt-3′ | 21 | 884 | 403 |

N. CG161579-01: Dimethylaniline Monooxygenase (N-Oxide-Forming)-Like Protein.

Expression of gene CG161579-01 was assessed using the primer-probe set Ag5741, described in Table NA.

TABLE NA

Probe Name Ag5741

| | | | | |
|---|---|---|---|---|
| | | | | SEQ ID |
| Primers | Sequenes | Length | Start Position | No |

| | | | | |
|---|---|---|---|---|
| Forward | 5′-gttcacagaaacagatgccatt-3′ | 22 | 970 404 | |
| Probe | TET-5′-tcaatattctcctctacagtgctgtcttca-3′-TAMRA | 30 | 994 | 405 |
| Reverse | 5′-tcctgtagcaaagatgacaaca-3′ | 22 | 1024 | 406 |

O. CG161650-01: Cytochrome c Oxidase Polypeptide VIc-Like Protein.

Expression of gene CG161650-01 was assessed using the primer-probe set Ag5744, described in Table OA.

TABLE OA

Probe Name Ag5744

| | | | | |
|---|---|---|---|---|
| | | | | SEQ ID |
| Primers | Sequencs | Length | Start Position | No |

| | | | | |
|---|---|---|---|---|
| Forward | 5′-aggttgacatacctataaaggacagtaac-3′ | 29 | 28 | 407 |
| Probe | TET-5′-ccatggcttccaccgctttgg-3′-TAMRA | 21 | 59 | 408 |
| Reverse | 5′-tttgtattctgaaagataccagcctt-3′ | 26 | 259 | 409 |

P. CG161733-01: Axonemal Dynein Heavy Chain-Like Protein.

Expression of gene CG161733-01 was assessed using the primer-probe set Ag5755, described in Table PA. Results of the RTQ-PCR runs are shown in Tables PB, PC and PD.

TABLE PA

Probe Name Ag5755

| | | | | |
|---|---|---|---|---|
| | | | | SEQ ID |
| Primers | Sequenes | Length | Start Position | No |

| | | | | |
|---|---|---|---|---|
| Forward | 5′-tcatttcaaccatcagaacatg-3′ | 22 | 2814 | 410 |
| Probe | TET-5′-tggcctctagtgtattccttgtaactttca-3′-TAMRA | 30 | 2846 | 411 |
| Reverse | 5′-tgagaggaatgaatacgtttgc-3′ | 22 | 2879 | 412 |

TABLE PB
CNS_neurodegeneration_v1.0

| | Rel. |
|---|---|
| | Exp. (%) |
| | Ag5755, |
| | Run |
| Tissue Name | 247026782 |
| AD 1 Hippo | 7.4 |
| AD 2 Hippo | 54.7 |
| AD 3 Hippo | 16.3 |
| AD 4 Hippo | 21.0 |
| AD 5 hippo | 51.1 |
| AD 6 Hippo | 42.9 |
| Control 2 Hippo | 40.9 |
| Control 4 Hippo | 98.6 |
| Control (Path) 3 Hippo | 75.3 |
| AD 1 Temporal Ctx | 18.6 |
| AD 2 Temporal Ctx | 37.6 |
| AD 3 Temporal Ctx | 4.3 |
| AD 4 Temporal Ctx | 43.5 |
| AD 5 Inf Temporal Ctx | 27.4 |
| AD 5 Sup Temporal Ctx | 80.1 |
| AD 6 Inf Temporal Ctx | 0.0 |
| AD 6 Sup Temporal Ctx | 29.5 |
| Control 1 Temporal Ctx | 14.0 |
| Control 2 Temporal Ctx | 29.5 |
| Control 3 Temporal Ctx | 20.0 |
| Control 4 Temporal Ctx | 19.9 |
| Control (Path) 1 Temporal Ctx | 81.8 |
| Control (Path) 2 Temporal Ctx | 40.6 |
| Control (Path) 3 Temporal Ctx | 14.9 |
| Control (Path) 4 Temporal Ctx | 40.3 |
| AD 1 Occipital Ctx | 9.4 |
| AD 2 Occipital Ctx (Missing) | 0.0 |
| AD 3 Occipital Ctx | 7.0 |
| AD 4 Occipital Ctx | 24.5 |
| AD 5 Occipital Ctx | 10.4 |
| AD 6 Occipital Ctx | 33.4 |
| Control 1 Occipital Ctx | 2.9 |
| Control 2 Occipital Ctx | 20.3 |
| Control 3 Occipital Ctx | 17.2 |
| Control 4 Occipital Ctx | 24.5 |
| Control (Path) 1 Occipital Ctx | 100.0 |
| Control (Path) 2 Occipital Ctx | 15.6 |
| Control (Path) 3 Occipital Ctx | 4.8 |
| Control (Path) 4 Occipital Ctx | 18.8 |
| Control 1 Parietal Ctx | 3.8 |
| Control 2 Parietal Ctx | 30.6 |
| Control 3 Parietal Ctx | 8.2 |
| Control (Path) 1 Parietal Ctx | 84.1 |
| Control (Path) 2 Parietal Ctx | 28.3 |
| Control (Path) 3 Parietal Ctx | 7.2 |
| Control (Path) 4 Parietal Ctx | 35.4 |

TABLE PC
General_screening_panel_v1.5

| | Rel. |
|---|---|
| | Exp. (%) |
| | Ag5755, |
| | Run |
| Tissue Name | 246263907 |
| Adipose | 0.0 |
| Melanoma* Hs688(A).T | 3.6 |
| Melanoma* Hs688(B).T | 3.2 |
| Melanoma* M14 | 0.0 |
| Melanoma* LOXIMVI | 4.6 |
| Melanoma* SK-MEL-5 | 0.2 |
| Squamous cell carcinoma SCC-4 | 2.7 |
| Testis Pool | 5.0 |
| Prostate ca.* (bone met) PC-3 | 1.5 |
| Prostate Pool | 9.0 |
| Placenta | 0.0 |
| Uterus Pool | 0.3 |
| Ovarian ca. OVCAR-3 | 6.5 |
| Ovarian ca. SK-OV-3 | 2.5 |
| Ovarian ca. OVCAR-4 | 0.3 |
| Ovarian ca. OVCAR-5 | 24.7 |
| Ovarian ca. IGROV-1 | 48.6 |
| Ovarian ca. OVCAR-8 | 3.3 |
| Ovary | 0.6 |
| Breast ca. MCF-7 | 0.0 |
| Breast ca. MDA-MB-231 | 9.3 |
| Breast ca. BT 549 | 16.3 |
| Breast ca. T47D | 0.8 |
| Breast ca. MDA-N | 0.0 |
| Breast Pool | 1.0 |
| Trachea | 47.3 |
| Lung | 0.0 |
| Fetal Lung | 72.2 |
| Lung ca. NCI-N417 | 0.0 |
| Lung ca. LX-1 | 2.2 |
| Lung ca. NCI-H146 | 0.0 |
| Lung ca. SHP-77 | 18.0 |
| Lung ca. A549 | 6.6 |
| Lung ca. NCI-H526 | 0.0 |
| Lung ca. NCI-H23 | 2.7 |
| Lung ca. NCI-H460 | 3.3 |
| Lung ca. HOP-62 | 23.2 |
| Lung ca. NCI-H522 | 1.8 |
| Liver | 0.0 |
| Fetal Liver | 0.0 |
| Liver ca. HepG2 | 0.0 |
| Kidney Pool | 1.6 |
| Fetal Kidney | 14.9 |
| Renal ca. 786-0 | 5.3 |
| Renal ca. A498 | 5.7 |
| Renal ca. ACHN | 13.0 |
| Renal ca. UO-31 | 8.2 |
| Renal ca. TK-10 | 17.0 |
| Bladder | 30.4 |
| Gastric ca. (liver met.) NCI-N87 | 23.8 |
| Gastric ca. KATO III | 0.3 |
| Colon ca. SW-948 | 1.3 |
| Colon ca. SW480 | 0.3 |
| Colon ca.* (SW480 met) SW620 | 0.2 |
| Colon ca. HT29 | 1.9 |
| Colon ca. HCT-116 | 0.0 |
| Colon ca. CaCo-2 | 0.0 |
| Colon cancer tissue | 0.3 |
| Colon ca. SW1116 | 0.0 |
| Colon ca. Colo-205 | 0.0 |
| Colon ca. SW-48 | 0.0 |
| Colon Pool | 1.5 |
| Small Intestine Pool | 2.1 |
| Stomach Pool | 0.3 |
| Bone Marrow Pool | 0.5 |
| Fetal Heart | 0.0 |
| Heart Pool | 0.0 |
| Lymph Node Pool | 0.8 |
| Fetal Skeletal Muscle | 0.7 |
| Skeletal Muscle Pool | 0.6 |
| Spleen Pool | 0.9 |
| Thymus Pool | 2.2 |
| CNS cancer (glio/astro) U87-MG | 4.2 |
| CNS cancer (glio/astro) U-118-MG | 6.1 |
| CNS cancer (neuro; met) SK-N-AS | 0.0 |
| CNS cancer (astro) SF-539 | 0.0 |
| CNS cancer (astro) SNB-75 | 1.0 |
| CNS cancer (glio) SNB-19 | 55.9 |
| CNS cancer (glio) SF-295 | 100.0 |
| Brain (Amygdala) Pool | 2.1 |
| Brain (cerebellum) | 2.2 |
| Brain (fetal) | 9.2 |
| Brain (Hippocampus) Pool | 0.7 |
| Cerebral Cortex Pool | 2.0 |
| Brain (Substantia nigra) Pool | 3.1 |
| Brain (Thalamus) Pool | 2.9 |
| Brain (whole) | 0.9 |
| Spinal Cord Pool | 2.8 |
| Adrenal Gland | 0.8 |
| Pituitary gland Pool | 13.1 |
| Salivary Gland | 0.0 |
| Thyroid (female) | 0.7 |
| Pancreatic ca. CAPAN2 | 10.4 |
| Pancreas Pool | 14.8 |

TABLE PD
Panel 4.1D

| | Rel. |
|---|---|
| | Exp. (%) |
| | Ag5755, |
| | Run |
| Tissue Name | 247283915 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 10.9 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 2.3 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 2.6 |
| HUVEC starved | 0.0 |
| HUVEC IL-1beta | 6.4 |
| HUVEC IFN gamma | 1.4 |
| HUVEC TNF alpha + IFN gamma | 0.0 |
| HUVEC TNF alpha + IL4 | 0.0 |
| HUVEC IL-11 | 7.1 |
| Lung Microvascular EC none | 46.7 |
| Lung Microvascular EC TNFalpha + IL-1beta | 5.8 |
| Microvascular Dermal EC none | 1.1 |
| Microsvasular Dermal EC TNFalpha + IL-1beta | 4.4 |
| Bronchial epithelium TNFalpha + IL1beta | 1.6 |
| Small airway epithelium none | 12.9 |
| Small airway epithelium TNFalpha + IL-1beta | 11.3 |
| Coronery artery SMC rest | 8.5 |
| Coronery artery SMC TNFalpha + IL-1beta | 10.3 |
| Astrocytes rest | 5.8 |
| Astrocytes TNFalpha + IL-1beta | 6.3 |
| KU-812 (Basophil) rest | 0.0 |
| KU-812 (Basophil) PMA/ionomycin | 1.1 |
| CCD1106 (Keratinocytes) none | 20.6 |
| CCD1106 (Keratinocytes) TNFalpha + IL-1beta | 7.5 |
| Liver cirrhosis | 19.3 |
| NCI-H292 none | 8.8 |
| NCI-H292 IL-4 | 9.6 |
| NCI-H292 IL-9 | 17.2 |
| NCI-H292 IL-13 | 5.4 |
| NCI-H292 IFN gamma | 12.6 |
| HPAEC none | 0.0 |
| HPAEC TNF alpha + IL-1 beta | 15.4 |
| Lung fibroblast none | 73.2 |
| Lung fibroblast TNF alpha + IL-1 beta | 90.1 |
| Lung fibroblast IL-4 | 30.1 |
| Lung fibroblast IL-9 | 100.0 |
| Lung fibroblast IL-13 | 9.5 |
| Lung fibroblast IFN gamma | 72.2 |
| Dermal fibroblast CCD1070 rest | 9.7 |
| Dermal fibroblast CCD1070 TNF alpha | 15.6 |
| Dermal fibroblast CCD1070 IL-1 beta | 10.7 |
| Dermal fibroblast IFN gamma | 12.1 |
| Dermal fibroblast IL-4 | 1.3 |
| Dermal Fibroblasts rest | 6.6 |
| Neutrophils TNFa + LPS | 0.0 |
| Neutrophils rest | 0.0 |
| Colon | 0.0 |
| Lung | 0.0 |
| Thymus | 0.0 |
| Kidney | 20.4 |

CNS_neurodegeneration_v1.0 Summary: Ag5755 No differential expression of this gene was detected between Alzheimer's diseased postmortem brains and those of non-demented controls in this experiment. However, this panel confirms the expression of this gene at low levels in the brains of an independent group of individuals. Therefore, therapeutic modulation of this gene product may be useful in the treatment of central nervous system disorders such as Parkinson's disease, epilepsy, multiple sclerosis, schizophrenia and depression.

General_screening_panel_v1.5 Summary: Ag5755 Highest expression of this gene is detected in a brain cancer SF-295 cell line (CT=30.6). Moderate to low expression of this gene is also seen in number of cell lines derived from brain, pancreatic, gastric, renal, lung, breast, and ovarian cancers. Thus, expression of this gene could be used as a marker to detect the presence of these cancers. Furthermore, therapeutic modulation of the expression or function of this gene may be effective in the treatment of brain, pancreatic, gastric, renal, lung, breast, and ovarian cancers.

Low expression of this gene is also detected in pituatary gland and pancreas. Therefore, therapeutic modulation of the activity of this gene may prove useful in the treatment of endocrine/metabolically related diseases, such as obesity and diabetes.

Moderate to low expression of this gene is also seen in fetal lung and kidney. Interestingly, this gene is expressed at much higher levels in fetal (CTs=31-33) when compared to adult lung and kidney (CTs=36-40). This observation suggests that expression of this gene can be used to distinguish fetal from adult lung and kidney. In addition, the relative overexpression of this gene in fetal tissue suggests that the protein product may enhance lung and kidney growth or development in the fetus and thus may also act in a regenerative capacity in the adult. Therefore, therapeutic modulation of the protein encoded by this gene could be useful in treatment of lung and kidney related diseases.

Panel 4.1D Summary: Ag5755 Highest expression of this gene is detected in IL-9 activated lung fibroblast (CT=33). Low expression of this gene is restricted to resting and activated lung fibroblasts and resting lung microvascular endothelial cells. The expression of this gene in cells derived from or within the lung suggests that this gene may be involved in normal conditions as well as pathological and inflammatory lung disorders that include chronic obstructive pulmonary disease, asthma, allergy and emphysema. Therefore, therapeutic modulation of this gene or its protein product may be useful in the treatment of chronic obstructive pulmonary disease, asthma, allergy and emphysema.

Q. CG161762-01: Voltage-Dependent Anion-Selective Channel Protein 3-Like Protein.

Expression of gene CG161762-01 was assessed using the primer-probe set Ag7848, described in Table QA. Results of the RTQ-PCR runs are shown in Tables QB, QC and QD. Please note that CG161762-01 represents a full length physical clone.

TABLE QA

Probe Name Ag7848

| | | | | |
|---|---|---|---|---|
| | | | | SEQ ID |
| Primers | Sequeces | Length | Start Position | No |

| | | | | |
|---|---|---|---|---|
| Forward | 5′-acttccagctgcacacacat-3′ | 20 | 548 | 413 |
| Probe | TET-5′-ctccaaattcagtgccatcgttcac-3′-TAMRA | 25 | 568 | 414 |
| Reverse | 5′-attatttactttagccttctggtagatagaac-4′ | 32 | 593 | 415 |

TABLE QB
CNS_neurodegeneration_v1.0

| | Rel. |
|---|---|
| | Exp. (%) |
| | Ag7848, |
| | Run |
| Tissue Name | 316264622 |
| AD 1 Hippo | 13.5 |
| AD 2 Hippo | 31.0 |
| AD 3 Hippo | 6.1 |
| AD 4 Hippo | 5.8 |
| AD 5 Hippo | 98.6 |
| AD 6 Hippo | 62.9 |
| Control 2 Hippo | 31.2 |
| Control 4 Hippo | 9.5 |
| Control (Path) 3 Hippo | 8.4 |
| AD 1 Temporal Ctx | 8.8 |
| AD 2 Temporal Ctx | 30.4 |
| AD 3 Temporal Ctx | 3.9 |
| AD 4 Temporal Ctx | 12.1 |
| AD 5 Inf Temporal Ctx | 100.0 |
| AD 5 Sup Temporal Ctx | 38.2 |
| AD 6 Inf Temporal Ctx | 55.1 |
| AD 6 Sup Temporal Ctx | 58.2 |
| Control 1 Temporal Ctx | 5.6 |
| Control 2 Temporal Ctx | 57.8 |
| Control 3 Temporal Ctx | 13.7 |
| Control 3 Temporal Ctx | 7.0 |
| Control (Path) 1 Temporal Ctx | 68.3 |
| Control (Path) 2 Temporal Ctx | 37.9 |
| Control (Path) 3 Temporal Ctx | 5.1 |
| Control (Path) 4 Temporal Ctx | 30.1 |
| AD 1 Occipital Ctx | 14.6 |
| AD 2 Occipital Ctx (Missing) | 0.0 |
| AD 3 Occipital Ctx | 6.4 |
| AD 4 Occipital Ctx | 16.4 |
| AD 5 Occipital Ctx | 61.1 |
| AD 6 Occipital Ctx | 28.9 |
| Control 1 Occipital Ctx | 4.3 |
| Control 2 Occipital Ctx | 84.1 |
| Control 3 Occipital Ctx | 12.2 |
| Control 4 Occipital Ctx | 4.1 |
| Control (Path) 1 Occipital Ctx | 98.6 |
| Control (Path) 2 Occipital Ctx | 9.8 |
| Control (Path) 3 Occipital Ctx | 4.2 |
| Control (Path) 4 Occipital Ctx | 9.4 |
| Control 1 Parietal Ctx | 5.0 |
| Control 2 Parietal Ctx | 36.9 |
| Control 3 Parietal Ctx | 22.1 |
| Control (Path) 1 Parietal Ctx | 34.6 |
| Control (Path) 2 Parietal Ctx | 22.8 |
| Control (Path) 3 Parietal Ctx | 4.9 |
| Control (Path) 4 Parietal Ctx | 37.4 |

TABLE QC
General_screening_panel_v1.7

| | Rel. |
|---|---|
| | Exp. (%) |
| | Ag7848, |
| | Run |
| Tissue Name | 318010160 |
| Adipose | 19.8 |
| HUVEC | 17.0 |
| Melanoma* Hs688(A).T | 0.0 |
| Melanoma* Hs688(B).T | 11.4 |
| Melanoma (met) SK-MEL-5 | 32.3 |
| Testis | 9.2 |
| Prostate ca. (bone met) PC-3 | 0.5 |
| Prostate ca. DU145 | 18.3 |
| Prostate pool | 1.9 |
| Uterus pool | 0.5 |
| Ovarian ca. OVCAR-3 | 16.7 |
| Ovarian ca. (ascites) SK-OV-3 | 9.3 |
| Ovarian ca. OVCAR-4 | 27.5 |
| Ovarian ca. OVCAR-5 | 31.0 |
| Ovarian ca. IGROV-1 | 93.3 |
| Ovarian ca. OVCAR-8 | 100.0 |
| Ovary | 7.2 |
| Breast ca. MCF-7 | 7.8 |
| Breast ca. MDA-MB-231 | 32.1 |
| Breast ca. BT-549 | 42.6 |
| Breast ca. T47D | 25.3 |
| Breast pool | 0.8 |
| Trachea | 8.4 |
| Lung | 11.0 |
| Fetal Lung | 15.2 |
| Lung ca. NCI-N417 | 5.8 |
| Lung ca. LX-1 | 6.6 |
| Lung ca. NCI-H146 | 17.6 |
| Lung ca. SHP-77 | 64.6 |
| Lung ca. NCI-H23 | 41.2 |
| Lung ca. NCI-H460 | 37.6 |
| Lung ca. HOP-62 | 40.1 |
| Lung ca. NCI-H522 | 24.0 |
| Lung ca. DMS-114 | 26.8 |
| Liver | 2.9 |
| Fetal Liver | 6.0 |
| Kidney pool | 24.5 |
| Fetal Kidney | 7.7 |
| Renal ca. 786-0 | 70.7 |
| Renal ca. A498 | 3.3 |
| Renal ca. ACHN | 14.8 |
| Renal ca. UO-31 | 16.4 |
| Renal ca. TK-10 | 22.5 |
| Bladder | 10.3 |
| Gastric ca. (liver met.) NCI-N87 | 40.3 |
| Stomach | 0.3 |
| Colon ca. SW-948 | 51.8 |
| Colon ca. SW480 | 46.3 |
| Colon ca. (SW480 met) SW620 | 35.1 |
| Colon ca. HT29 | 37.9 |
| Colon ca. HCT-116 | 69.7 |
| Colon cancer tissue | 0.5 |
| Colon ca. SW1116 | 8.3 |
| Colon ca. Colo-205 | 5.8 |
| Colon ca. SW-48 | 11.0 |
| Colon | 11.3 |
| Small Intestine | 0.8 |
| Fetal Heart | 16.4 |
| Heart | 3.8 |
| Lymph Node pool 1 | 0.7 |
| Lymph Node pool 2 | 14.1 |
| Fetal Skeletal Muscle | 12.6 |
| Skeletal Muscle pool | 4.6 |
| Skeletal Muscle | 80.1 |
| Spleen | 2.7 |
| Thymus | 2.0 |
| CNS cancer (glio/astro) SF-268 | 8.2 |
| CNS cancer (glio/astro) T98G | 11.1 |
| CNS cancer (neuro; met) SK-N-AS | 18.3 |
| CNS cancer (astro) SF-539 | 37.4 |
| CNS cancer (astro) SNB-75 | 13.7 |
| CNS cancer (glio) SNB-19 | 23.0 |
| CNS cancer (glio) SF-295 | 8.0 |
| Brain (Amygdala) | 10.9 |
| Brain (Cerebellum) | 22.7 |
| Brain (Fetal) | 25.7 |
| Brain (Hippocampus) | 8.0 |
| Cerebral Cortex pool | 8.8 |
| Brain (Substantia nigra) | 3.7 |
| Brain (Thalamus) | 8.8 |
| Brain (Whole) | 26.2 |
| Spinal Cord | 4.4 |
| Adrenal Gland | 5.3 |
| Pituitary Gland | 6.6 |
| Salivary Gland | 3.2 |
| Thyroid | 24.3 |
| Pancreatic ca. PANC-1 | 4.2 |
| Pancreas pool | 1.6 |

TABLE QD
Panel 4.1D

| | Rel. |
|---|---|
| | Exp. (%) |
| | Ag7848, |
| | Run |
| Tissue Name | 313918253 |
| Secondary Th1 act | 58.6 |
| Secondary Th2 act | 100.0 |
| Secondary Tr1 act | 27.4 |
| Secondary Th1 rest | 2.9 |
| Secondary Th2 rest | 4.4 |
| Secondary Tr1 rest | 2.7 |
| Primary Th1 act | 11.8 |
| Primary Th2 act | 49.0 |
| Primary Tr1 act | 57.4 |
| Primary Th1 rest | 4.0 |
| Primary Th2 rest | 3.3 |
| Primary Tr1 rest | 2.3 |
| CD45RA CD4 lymphocyte act | 41.2 |
| CD45RO CD4 lymphocyte act | 60.3 |
| CD8 lymphocyte act | 20.6 |
| Secondary CD8 lymphocyte rest | 13.5 |
| Secondary CD8 lymphocyte act | 6.3 |
| CD4 lymphocyte none | 1.8 |
| 2ry Th1/Th2/Tr1_anti-CD95 CH11 | 6.6 |
| LAK cells rest | 14.7 |
| LAK cells IL-2 | 12.7 |
| LAK cells IL-2 + IL-12 | 2.1 |
| LAK cells IL-2 + IFN gamma | 8.5 |
| LAK cells IL-2 + IL-18 | 10.9 |
| LAK cells PMA/ionomycin | 16.2 |
| NK Cells IL-2 rest | 37.1 |
| Two Way MLR 3 day | 9.2 |
| Two Way MLR 5 day | 6.9 |
| Two Way MLR 7 day | 7.9 |
| PBMC rest | 2.4 |
| PBMC PWM | 14.7 |
| PBMC PHA-L | 28.9 |
| Ramos (B cell) none | 7.4 |
| Ramos (B cell) ionomycin | 25.7 |
| B lymphocytes PWM | 23.0 |
| B lymphocytes CD40L and IL-4 | 34.4 |
| EOL-1 dbcAMP | 65.5 |
| EOL-1 dbcAMP PMA/ionomycin | 8.6 |
| Dendritic cells none | 16.5 |
| Dendritic cells LPS | 16.3 |
| Dendritic cells anti-CD40 | 8.8 |
| Monocytes rest | 2.9 |
| Monocytes LPS | 13.9 |
| Macrophages rest | 7.4 |
| Macrophages LPS | 7.3 |
| HUVEC none | 28.7 |
| HUVEC starved | 42.0 |
| HUVEC IL-1beta | 48.3 |
| HUVEC IFN gamma | 28.1 |
| HUVEC TNF alpha + IFN gamma | 9.5 |
| HUVEC TNF alpha + IL4 | 22.8 |
| HUVEC IL-11 | 15.0 |
| Lung Microvascular EC none | 45.7 |
| Lung Microvascular EC TNFalpha + IL-1beta | 8.9 |
| Microvascular Dermal EC none | 9.7 |
| Microsvasular Dermal EC TNFalpha + IL-1beta | 4.5 |
| Bronchial epithelium TNFalpha + IL1beta | 8.0 |
| Small airway epithelium none | 10.7 |
| Small airway epithelium TNFalpha + IL-1beta | 29.7 |
| Coronery artery SMC rest | 20.7 |
| Coronery artery SMC TNFalpha + IL-1beta | 14.3 |
| Astrocytes rest | 7.7 |
| Astrocytes TNFalpha + IL-1beta | 1.8 |
| KU-812 (Basophil) rest | 42.0 |
| KU-812 (Basophil) PMA/ionomycin | 59.0 |
| CCD1106 (Keratinocytes) none | 27.2 |
| CCD1106 (Keratinocytes) TNFalpha + IL-1beta | 7.1 |
| Liver cirrhosis | 3.4 |
| NCI-H292 none | 33.7 |
| NCI-H292 IL-4 | 33.4 |
| NCI-H292 IL-9 | 68.8 |
| NCI-H292 IL-13 | 35.6 |
| NCI-H292 IFN gamma | 15.5 |
| HPAEC none | 11.7 |
| HPAEC TNF alpha + IL-1 beta | 25.2 |
| Lung fibroblast none | 11.0 |
| Lung fibroblast TNF alpha + IL-1 beta | 11.4 |
| Lung fibroblast IL-4 | 10.9 |
| Lung fibroblast IL-9 | 11.9 |
| Lung fibroblast IL-13 | 6.8 |
| Lung fibroblast IFN gamma | 22.5 |
| Dermal fibroblast CCD1070 rest | 40.1 |
| Dermal fibroblast CCD1070 TNF alpha | 75.8 |
| Dermal fibroblast CCD1070 IL-1 beta | 23.8 |
| Dermal fibroblast IFN gamma | 15.0 |
| Dermal fibroblast IL-4 | 21.6 |
| Dermal Fibroblasts rest | 16.7 |
| Neutrophils TNFa + LPS | 0.7 |
| Neutrophils rest | 4.3 |
| Colon | 2.7 |
| Lung | 1.8 |
| Thymus | 2.5 |
| Kidney | 15.3 |

CNS_neurodegeneration_v1.0 Summary: Ag7848 This panel confirms the expression of this gene at low levels in the brains of an independent group of individuals. However, no differential expression of this gene was detected between Alzheimer's diseased postmortem brains and those of non-demented controls in this experiment. Please see Panel 1.7 for a discussion of this gene in treatment of central nervous system disorders.

General_screening_panel_v1.7 Summary: Ag7848 Highest expression of this gene is detected in OVCAR-8 (CT=23.4). High expression of this gene is also seen in cluster of cancer cell lines derived from pancreatic, gastric, colon, lung, liver, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, melanoma and brain cancers.

Among tissues with metabolic or endocrine function, this gene is expressed at high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 4.1D Summary: Ag7848 Highest expression of this gene is detected in activated secondary Th2 cells (CT=28.4). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.7 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

R. CG163937-01: Diamine N-acetyltransferase Like Protein.

Expression of gene CG163937-01 was assessed using the primer-probe sets Ag4716 and Ag5877, described in Tables RA and RB. Results of the RTQ-PCR runs are shown in Tables RC, RD, RE, RF and RG.

TABLE RA

Probe Name Ag4716

| | | | | |
|---|---|---|---|---|
| | | | | SEQ ID |
| Primers | Sequence | Length | Start Position | No |

| | | | | |
|---|---|---|---|---|
| Forward | 5′-tgccaaagcctctataatcact-3′ | 22 | 623 | 416 |
| Probe | TET-5′-catcacgaagaagtcctcaagatacaa-3′-TAMRA | 27 | 596 | 417 |
| Reverse | 5′-attttacctatgacccgtggat-3′ | 22 | 564 | 418 |

TABLE RB

Probe Name Ag5877

| | | | | |
|---|---|---|---|---|
| | | | | SEQ ID |
| Primers | Sequenes | Length | Start Position | No |

| | | | | |
|---|---|---|---|---|
| Forward | 5′-aagaggtgcttctgatctgtcc-3′ | 22 | 757 | 419 |
| Probe | TET-5′-tgaagagggttggagactgttcaagatcg-3′-TAMRA | 29 | 781 | 420 |
| Reverse | 5′-catctacagcagcactcctcac-3′ | 22 | 844 | 421 |

TABLE RC
AI_comprehensive_panel_v1.0

| | Rel. |
|---|---|
| | Exp. (%) |
| | Ag4716, |
| | Run |
| Tissue Name | 244333632 |
| 110967 COPD-F | 12.1 |
| 110980 COPD-F | 2.6 |
| 110968 COPD-M | 22.8 |
| 110977 COPD-M | 8.7 |
| 110989 Emphysema-F | 36.9 |
| 110992 Emphysema-F | 20.9 |
| 110993 Emphysema-F | 16.3 |
| 110994 Emphysema-F | 4.7 |
| 110995 Emphysema-F | 57.8 |
| 110996 Emphysema-F | 9.4 |
| 110997 Asthma-M | 6.0 |
| 111001 Asthma-F | 5.4 |
| 111002 Asthma-F | 19.3 |
| 111003 Atopic Asthma-F | 11.5 |
| 111004 Atopic Asthma-F | 31.0 |
| 111005 Atopic Asthma-F | 17.1 |
| 111006 Atopic Asthma-F | 2.2 |
| 111417 Allergy-M | 9.3 |
| 112347 Allergy-M | 0.7 |
| 112349 Normal Lung-F | 0.3 |
| 112357 Normal Lung-F | 15.4 |
| 112354 Normal Lung-M | 9.0 |
| 112374 Crohns-F | 16.3 |
| 112389 Match Control Crohns-F | 2.1 |
| 112375 Crohns-F | 9.7 |
| 112732 Match Control Crohns-F | 25.9 |
| 112725 Crohns-M | 6.4 |
| 112387 Match Control Crohns-M | 7.6 |
| 112378 Crohns-M | 0.5 |
| 112390 Match Control Crohns-M | 10.6 |
| 112726 Crohns-M | 14.2 |
| 112731 Match Control Crohns-M | 7.4 |
| 112380 Ulcer Col-F | 7.1 |
| 112734 Match Control Ulcer Col-F | 71.2 |
| 112384 Ulcer Col-F | 44.1 |
| 112737 Match Control Ulcer Col-F | 15.5 |
| 112386 Ulcer Col-F | 6.6 |
| 112738 Match Control Ulcer Col-F | 11.1 |
| 112381 Ulcer Col-M | 0.5 |
| 112735 Match Control Ulcer Col-M | 10.2 |
| 112382 Ulcer Col-M | 6.6 |
| 112394 Match Control Ulcer Col-M | 6.6 |
| 112383 Ulcer Col-M | 30.1 |
| 112736 Match Control Ulcer Col-M | 2.9 |
| 112423 Psoriasis-F | 11.2 |
| 112427 Match Control Psoriasis-F | 31.2 |
| 112418 Psoriasis-M | 25.9 |
| 112723 Match Control Psoriasis-M | 1.4 |
| 112419 Psoriasis-M | 37.6 |
| 112424 Match Control Psoriasis-M | 21.9 |
| 112420 Psoriasis-M | 60.3 |
| 112425 Match Control Psoriasis-M | 10.7 |
| 104689 (MF) OA Bone-Backus | 23.8 |
| 104690 (MF) Adj “Normal” Bone-Backus | 8.4 |
| 104691 (MF) OA Synovium-Backus | 27.9 |
| 104692 (BA) OA Cartilage-Backus | 15.2 |
| 104694 (BA) OA Bone-Backus | 27.7 |
| 104695 (BA) Adj “Normal” Bone-Backus | 20.3 |
| 104696 (BA) OA Synovium-Backus | 34.2 |
| 104700 (SS) OA Bone-Backus | 12.9 |
| 104701 (SS) Adj “Normal” Bone-Backus | 15.8 |
| 104702 (SS) OA Synovium-Backus | 25.5 |
| 117093 OA Cartilage Rep7 | 8.7 |
| 112672 OA Bone5 | 64.6 |
| 112673 OA Synovium5 | 31.4 |
| 112674 OA Synovial Fluid cells5 | 37.1 |
| 117100 OA Cartilage Rep14 | 8.1 |
| 112756 OA Bone9 | 100.0 |
| 112757 OA Synovium9 | 1.8 |
| 112758 OA Synovial Fluid Cells9 | 8.3 |
| 117125 RA Cartilage Rep2 | 5.8 |
| 113492 Bone2 RA | 37.9 |
| 113493 Synovium2 RA | 15.7 |
| 113494 Syn Fluid Cells RA | 26.1 |
| 113499 Cartilage4 RA | 58.2 |
| 113500 Bone4 RA | 63.7 |
| 113501 Synovium4 RA | 57.0 |
| 113502 Syn Fluid Cells4 RA | 33.4 |
| 113495 Cartilage3 RA | 22.4 |
| 113496 Bone3 RA | 19.6 |
| 113497 Synovium3 RA | 11.8 |
| 113498 Syn Fluid Cells3 RA | 30.8 |
| 117106 Normal Cartilage Rep20 | 1.1 |
| 113663 Bone3 Normal | 1.2 |
| 113664 Synovium3 Normal | 0.2 |
| 113665 Syn Fluid Cells3 Normal | 1.1 |
| 117107 Normal Cartilage Rep22 | 3.2 |
| 113667 Bone4 Normal | 27.9 |
| 113668 Synovium4 Normal | 42.3 |
| 113669 Syn Fluid Cells4 Normal | 39.2 |

TABLE RD
General_screening_panel_v1.4

| | Rel. |
|---|---|
| | Exp. (%) |
| | Ag4716, |
| | Run |
| Tissue Name | 214237609 |
| Adipose | 28.5 |
| Melanoma* Hs688(A).T | 3.1 |
| Melanoma* Hs688(B).T | 4.5 |
| Melanoma* M14 | 36.9 |
| Melanoma* LOXIMVI | 1.3 |
| Melanoma* SK-MEL-5 | 19.5 |
| Squamous cell carcinoma SCC-4 | 5.1 |
| Testis Pool | 13.8 |
| Prostate ca.* (bone met) PC-3 | 16.4 |
| Prostate Pool | 4.6 |
| Placenta | 30.6 |
| Uterus Pool | 2.2 |
| Ovarian ca. OVCAR-3 | 6.2 |
| Ovarian ca. SK-OV-3 | 7.8 |
| Ovarian ca. OVCAR-4 | 5.7 |
| Ovarian ca. OVCAR-5 | 31.9 |
| Ovarian ca. IGROV-1 | 63.7 |
| Ovarian ca. OVCAR-8 | 25.3 |
| Ovary | 5.8 |
| Breast ca. MCF-7 | 2.8 |
| Breast ca. MDA-MB-231 | 6.2 |
| Breast ca. BT 549 | 21.2 |
| Breast ca. T47D | 47.3 |
| Breast ca. MDA-N | 41.8 |
| Breast Pool | 7.1 |
| Trachea | 30.8 |
| Lung | 3.0 |
| Fetal Lung | 56.6 |
| Lung ca. NCI-N417 | 0.3 |
| Lung ca. LX-1 | 100.0 |
| Lung ca. NCI-H146 | 0.4 |
| Lung ca. SHP-77 | 1.4 |
| Lung ca. A549 | 54.0 |
| Lung ca. NCI-H526 | 0.8 |
| Lung ca. NCI-H23 | 84.7 |
| Lung ca. NCI-H460 | 9.5 |
| Lung ca. HOP-62 | 6.3 |
| Lung ca. NCI-H522 | 6.9 |
| Liver | 3.1 |
| Fetal Liver | 21.2 |
| Liver ca. HepG2 | 27.4 |
| Kidney Pool | 8.7 |
| Fetal Kidney | 4.8 |
| Renal ca. 786-0 | 7.9 |
| Renal ca. A498 | 5.3 |
| Renal ca. ACHN | 4.0 |
| Renal ca. UO-31 | 31.0 |
| Renal ca. TK-10 | 13.6 |
| Bladder | 74.7 |
| Gastric ca. (liver met.) NCI-N87 | 15.2 |
| Gastric ca. KATO III | 76.8 |
| Colon ca. SW-948 | 5.0 |
| Colon ca. SW480 | 28.9 |
| Colon ca.* (SW480 met) SW620 | 51.8 |
| Colon ca. HT29 | 4.5 |
| Colon ca. HCT-116 | 28.7 |
| Colon ca. CaCo-2 | 14.0 |
| Colon cancer tissue | 52.1 |
| Colon ca. SW1116 | 1.2 |
| Colon ca. Colo-205 | 9.3 |
| Colon ca. SW-48 | 3.5 |
| Colon Pool | 4.8 |
| Small Intestine Pool | 3.3 |
| Stomach Pool | 14.3 |
| Bone Marrow Pool | 3.8 |
| Fetal Heart | 1.6 |
| Heart Pool | 2.6 |
| Lymph Node Pool | 7.4 |
| Fetal Skeletal Muscle | 1.4 |
| Skeletal Muscle Pool | 1.6 |
| Spleen Pool | 13.9 |
| Thymus Pool | 12.9 |
| CNS cancer (glio/astro) U87-MG | 24.8 |
| CNS cancer (glio/astro) U-118-MG | 19.5 |
| CNS cancer (neuro; met) SK-N-AS | 2.2 |
| CNS cancer (astro) SF-539 | 2.7 |
| CNS cancer (astro) SNB-75 | 57.8 |
| CNS cancer (glio) SNB-19 | 62.0 |
| CNS cancer (glio) SF-295 | 72.2 |
| Brain (Amygdala) Pool | 2.9 |
| Brain (cerebellum) | 1.7 |
| Brain (fetal) | 5.1 |
| Brain (Hippocampus) Pool | 6.3 |
| Cerebral Cortex Pool | 5.8 |
| Brain (Substantia nigra) Pool | 6.6 |
| Brain (Thalamus) Pool | 6.1 |
| Brain (whole) | 4.7 |
| Spinal Cord Pool | 6.7 |
| Adrenal Gland | 11.7 |
| Pituitary gland Pool | 2.6 |
| Salivary Gland | 5.0 |
| Thyroid (female) | 23.8 |
| Pancreatic ca. CAPAN2 | 10.8 |
| Pancreas Pool | 13.4 |

TABLE RE
General_screening_panel_v1.5

| | Rel. |
|---|---|
| | Exp. (%) |
| | Ag5877, |
| | Run |
| Tissue Name | 248204736 |
| Adipose | 41.2 |
| Melanoma* Hs688(A).T | 3.9 |
| Melanoma* Hs688(B).T | 5.5 |
| Melanoma* M14 | 40.3 |
| Melanoma* LOXIMVI | 1.8 |
| Melanoma* SK-MEL-5 | 20.6 |
| Squamous cell carcinoma SCC-4 | 7.1 |
| Testis Pool | 7.5 |
| Prostate ca.* (bone met) PC-3 | 16.4 |
| Prostate Pool | 17.0 |
| Placenta | 38.2 |
| Uterus Pool | 7.4 |
| Ovarian ca. OVCAR-3 | 6.0 |
| Ovarian ca. SK-OV-3 | 8.8 |
| Ovarian ca. OVCAR-4 | 3.2 |
| Ovarian ca. OVCAR-5 | 22.5 |
| Ovarian ca. IGROV-1 | 67.8 |
| Ovarian ca. OVCAR-8 | 22.1 |
| Ovary | 10.7 |
| Breast ca. MCF-7 | 3.3 |
| Breast ca. MDA-MB-231 | 9.0 |
| Breast ca. BT 549 | 18.3 |
| Breast ca. T47D | 14.2 |
| Breast ca. MDA-N | 33.0 |
| Breast Pool | 13.8 |
| Trachea | 38.2 |
| Lung | 4.1 |
| Fetal Lung | 95.9 |
| Lung ca. NCI-N417 | 0.3 |
| Lung ca. LX-1 | 84.1 |
| Lung ca. NCI-H146 | 0.5 |
| Lung ca. SHP-77 | 1.9 |
| Lung ca. A549 | 43.8 |
| Lung ca. NCI-H526 | 0.7 |
| Lung ca. NCI-H23 | 77.9 |
| Lung ca. NCI-H460 | 9.9 |
| Lung ca. HOP-62 | 5.8 |
| Lung ca. NCI-H522 | 8.6 |
| Liver | 3.3 |
| Fetal Liver | 17.0 |
| Liver ca. HepG2 | 21.3 |
| Kidney Pool | 15.3 |
| Fetal Kidney | 8.5 |
| Renal ca. 786-0 | 8.1 |
| Renal ca. A498 | 6.3 |
| Renal ca. ACHN | 2.6 |
| Renal ca. UO-31 | 32.1 |
| Renal ca. TK-10 | 15.7 |
| Bladder | 100.0 |
| Gastric ca. (liver met.) NCI-N87 | 17.1 |
| Gastric ca. KATO III | 58.2 |
| Colon ca. SW-948 | 6.6 |
| Colon ca. SW480 | 30.8 |
| Colon ca.* (SW480 met) SW620 | 62.4 |
| Colon ca. HT29 | 4.3 |
| Colon ca. HCT-116 | 34.9 |
| Colon ca. CaCo-2 | 12.4 |
| Colon cancer tissue | 59.0 |
| Colon ca. SW1116 | 1.5 |
| Colon ca. Colo-205 | 6.3 |
| Colon ca. SW-48 | 4.2 |
| Colon Pool | 8.4 |
| Small Intestine Pool | 2.4 |
| Stomach Pool | 22.1 |
| Bone Marrow Pool | 6.4 |
| Fetal Heart | 3.4 |
| Heart Pool | 4.5 |
| Lymph Node Pool | 12.7 |
| Fetal Skeletal Muscle | 1.5 |
| Skeletal Muscle Pool | 2.7 |
| Spleen Pool | 20.6 |
| Thymus Pool | 21.0 |
| CNS cancer (glio/astro) U87-MG | 20.9 |
| CNS cancer (glio/astro) U-118-MG | 15.5 |
| CNS cancer (neuro; met) SK-N-AS | 1.5 |
| CNS cancer (astro) SF-539 | 0.9 |
| CNS cancer (astro) SNB-75 | 74.2 |
| CNS cancer (glio) SNB-19 | 80.7 |
| CNS cancer (glio) SF-295 | 66.0 |
| Brain (Amygdala) Pool | 4.9 |
| Brain (cerebellum) | 3.4 |
| Brain (fetal) | 6.4 |
| Brain (Hippocampus) Pool | 8.3 |
| Cerebral Cortex Pool | 6.0 |
| Brain (Substantia nigra) Pool | 5.4 |
| Brain (Thalamus) Pool | 7.5 |
| Brain (whole) | 5.8 |
| Spinal Cord Pool | 9.2 |
| Adrenal Gland | 15.9 |
| Pituitary gland Pool | 5.6 |
| Salivary Gland | 4.3 |
| Thyroid (female) | 28.1 |
| Pancreatic ca. CAPAN2 | 13.7 |
| Pancreas Pool | 22.8 |

TABLE RF
Panel 4.1D

| | Rel. |
|---|---|
| | Exp. (%) |
| | Ag4716, |
| | Run |
| Tissue Name | 244337062 |
| Secondary Th1 act | 0.2 |
| Secondary Th2 act | 4.7 |
| Secondary Tr1 act | 1.0 |
| Secondary Th1 rest | 0.0 |
| Secondary Th2 rest | 0.2 |
| Secondary Tr1 rest | 0.1 |
| Primary Th1 act | 0.0 |
| Primary Th2 act | 2.1 |
| Primary Tr1 act | 1.3 |
| Primary Th1 rest | 0.1 |
| Primary Th2 rest | 0.5 |
| Primary Tr1 rest | 0.0 |
| CD45RA CD4 lymphocyte act | 4.0 |
| CD45RO CD4 lymphocyte act | 6.3 |
| CD8 lymphocyte act | 0.3 |
| Secondary CD8 lymphocyte rest | 2.1 |
| Secondary CD8 lymphocyte act | 0.3 |
| CD4 lymphocyte none | 0.1 |
| 2ry Th1/Th2/Tr1_anti-CD95 CH11 | 0.3 |
| LAK cells rest | 5.8 |
| LAK cells IL-2 | 0.8 |
| LAK cells IL-2 + IL-12 | 0.4 |
| LAK cells IL-2 + IFN gamma | 1.3 |
| LAK cells IL-2 + IL-18 | 0.4 |
| LAK cells PMA/ionomycin | 58.6 |
| NK Cells IL-2 rest | 3.8 |
| Two Way MLR 3 day | 5.3 |
| Two Way MLR 5 day | 0.7 |
| Two Way MLR 7 day | 2.0 |
| PBMC rest | 1.6 |
| PBMC PWM | 1.0 |
| PBMC PHA-L | 1.3 |
| Ramos (B cell) none | 0.2 |
| Ramos (B cell) ionomycin | 1.5 |
| B lymphocytes PWM | 2.6 |
| B lymphocytes CD40L and IL-4 | 4.2 |
| EOL-1 dbcAMP | 6.6 |
| EOL-1 dbcAMP PMA/ionomycin | 0.9 |
| Dendritic cells none | 6.7 |
| Dendritic cells LPS | 7.5 |
| Dendritic cells anti-CD40 | 0.8 |
| Monocytes rest | 2.0 |
| Monocytes LPS | 100.0 |
| Macrophages rest | 27.5 |
| Macrophages LPS | 20.2 |
| HUVEC none | 1.4 |
| HUVEC starved | 3.1 |
| HUVEC IL-1beta | 16.7 |
| HUVEC IFN gamma | 13.0 |
| HUVEC TNF alpha + IFN gamma | 3.5 |
| HUVEC TNF alpha + IL4 | 2.1 |
| HUVEC IL-11 | 3.6 |
| Lung Microvascular EC none | 4.0 |
| Lung Microvascular EC TNFalpha + IL-1beta | 4.9 |
| Microvascular Dermal EC none | 0.3 |
| Microsvasular Dermal EC TNFalpha + IL-1beta | 6.3 |
| Bronchial epithelium TNFalpha + IL1beta | 53.2 |
| Small airway epithelium none | 19.6 |
| Small airway epithelium TNFalpha + IL-1beta | 53.6 |
| Coronery artery SMC rest | 2.8 |
| Coronery artery SMC TNFalpha + IL-1beta | 10.0 |
| Astrocytes rest | 1.2 |
| Astrocytes TNFalpha + IL-1beta | 2.0 |
| KU-812 (Basophil) rest | 3.0 |
| KU-812 (Basophil) PMA/ionomycin | 5.0 |
| CCD1106 (Keratinocytes) none | 20.4 |
| CCD1106 (Keratinocytes) TNFalpha + IL-1beta | 14.0 |
| Liver cirrhosis | 6.0 |
| NCI-H292 none | 73.7 |
| NCI-H292 IL-4 | 71.2 |
| NCI-H292 IL-9 | 67.8 |
| NCI-H292 IL-13 | 74.2 |
| NCI-H292 IFN gamma | 20.6 |
| HPAEC none | 1.8 |
| HPAEC TNF alpha + IL-1 beta | 80.7 |
| Lung fibroblast none | 2.9 |
| Lung fibroblast TNF alpha + IL-1 beta | 17.2 |
| Lung fibroblast IL-4 | 1.1 |
| Lung fibroblast IL-9 | 2.1 |
| Lung fibroblast IL-13 | 0.2 |
| Lung fibroblast IFN gamma | 9.0 |
| Dermal fibroblast CCD1070 rest | 1.2 |
| Dermal fibroblast CCD1070 TNF alpha | 4.6 |
| Dermal fibroblast CCD1070 IL-1 beta | 4.4 |
| Dermal fibroblast IFN gamma | 2.9 |
| Dermal fibroblast IL-4 | 1.1 |
| Dermal Fibroblasts rest | 1.2 |
| Neutrophils TNFa + LPS | 95.3 |
| Neutrophils rest | 26.4 |
| Colon | 0.7 |
| Lung | 0.9 |
| Thymus | 1.1 |
| Kidney | 18.3 |

TABLE RG
Panel 5D

| | Rel. |
|---|---|
| | Exp. (%) |
| | Ag4716, |
| | Run |
| Tissue Name | 204245093 |
| 97457_Patient-02go_adipose | 33.7 |
| 97476_Patient-07sk_skeletal muscle | 12.7 |
| 97477_Patient-07ut_uterus | 11.6 |
| 97478_Patient-07pl_placenta | 54.3 |
| 97481_Patient-08sk_skeletal muscle | 4.2 |
| 97482_Patient-08ut_uterus | 8.2 |
| 97483_Patient-08pl_placenta | 41.2 |
| 97486_Patient-09sk_skeletal muscle | 1.3 |
| 97487_Patient-09ut_uterus | 6.7 |
| 97488_Patient-09pl_placenta | 49.3 |
| 97492_Patient-10ut_uterus | 12.8 |
| 97493_Patient-10pl_placenta | 100.0 |
| 97495_Patient-11go_adipose | 17.7 |
| 97496_Patient-11sk_skeletal muscle | 1.8 |
| 97497_Patient-11ut_uterus | 15.0 |
| 97498_Patient-11pl_placenta | 73.7 |
| 97500_Patient-12go_adipose | 27.5 |
| 97501_Patient-12sk_skeletal muscle | 8.4 |
| 97502_Patient-12ut_uterus | 27.7 |
| 97503_Patient-12pl_placenta | 75.8 |
| 94721_Donor 2 U - A_Mesenchymal Stem Cells | 5.1 |
| 94722_Donor 2 U - B_Mesenchymal Stem Cells | 3.5 |
| 94723_Donor 2 U - C_Mesenchymal Stem Cells | 3.7 |
| 94709_Donor 2 AM - A_adipose | 9.9 |
| 94710_Donor 2 AM - B_adipose | 8.7 |
| 94711_Donor 2 AM - C_adipose | 4.0 |
| 94712_Donor 2 AD - A_adipose | 11.6 |
| 94713_Donor 2 AD - B_adipose | 17.9 |
| 94714_Donor 2 AD - C_adipose | 12.5 |
| 94742_Donor 3 U - A_Mesenchymal Stem Cells | 2.3 |
| 94743_Donor 3 U - B_Mesenchymal Stem Cells | 2.0 |
| 94730_Donor 3 AM - A_adipose | 13.9 |
| 94731_Donor 3 AM - B_adipose | 8.1 |
| 94732_Donor 3 AM - C_adipose | 8.8 |
| 94733_Donor 3 AD - A_adipose | 19.1 |
| 94734_Donor 3 AD - B_adipose | 10.6 |
| 94735_Donor 3 AD - C_adipose | 13.5 |
| 77138_Liver_HepG2untreated | 20.9 |
| 73556_Heart_Cardiac stromal cells (primary) | 4.4 |
| 81735_Small Intestine | 23.0 |
| 72409_Kidney_Proximal Convoluted Tubule | 8.1 |
| 82685_Small intestine_Duodenum | 28.3 |
| 90650_Adrenal_Adrenocortical adenoma | 8.0 |
| 72410_Kidney_HRCE | 28.1 |
| 72411_Kidney_HRE | 26.1 |
| 73139_Uterus_Uterine smooth muscle cells | 1.8 |

AI_comprehensive panel_v1.0 Summary: Ag4716 This gene is expressed at moderate to high levels in the majority of tissues on this panel, with highest expression in an osteoarthritic bone sample (CT=26.6). Clusters of higher expression of this gene are associated with samples from osteoarthritis and rheumatoid arthritis patients. Therefore, therapeutic modulation of the activity of this gene or its protein product, through the use of small molecule drugs, protein therapeutics or antibodies, might be beneficial in the treatment of arthritis. Please see Panel 4.1D for additional discussion of the potential relevance of this gene in immune response.

General_screening_panel_v1.4 Summary: Ag4716 This gene is expressed at moderate to high levels in all of the tissues on this panel, with highest expression in a lung cancer cell line (CT=24.2). Interestingly, expression of this gene is higher in fetal lung and lung cancer cell lines when compared to adult lung. Expression of this gene is also upregulated in colon, brain, breast and ovarian cancer cell lines when compared to normal colon, brain, breast and ovary. Therefore, therapeutic modulation of the activity of this gene or its protein product, through the use of small molecule drugs, protein therapeutics or antibodies, might be beneficial in the treatment of lung, colon, brain, ovarian and breast cancers.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

General_screening_panel_v1.5 Summary: Ag5877 Expression of this gene is highest in bladder (CT=23.6). This gene is expressed at moderate to high levels in all of the tissues on this panel, consistent with what is observed in Panel 1.4. Interestingly, expression of this gene is higher in fetal lung (CT=23.7)and a subset of lung cancer cell lines (CTs=24) when compared to adult lung (CT=28.2). Expression of this gene is also upregulated in colon cancer cell lines (CTs=24) when compared to normal colon (CT=27.2). Therefore, therapeutic modulation of the activity of this gene or its protein product, through the use of small molecule drugs, protein therapeutics or antibodies, might be beneficial in the treatment of lung and colon cancer. Please see Panel 1.4 for additional discussion of the potential relevance of this gene in human disease.

Panel 4.1D Summary: Ag4716 Expression of this gene is highest in LPS-treated monocytes (CT=25.8), with lower expression in resting monocytes (CT=31.4). Therefore, expression of this gene could be used to distinguish resting and activated monocytes. The expression of this transcript in LPS-treated monocytes, cells that play a crucial role in linking innate immunity to adaptive immunity, suggests a role for this gene product in initiating inflammatory reactions. Thus, therapeutic modulation of the activity of this gene or its protein product may reduce or prevent early stages of inflammation and reduce the severity of inflammatory diseases such as psoriasis, asthma, inflammatory bowel disease, rheumatoid arthritis, osteoarthritis and other lung inflammatory diseases.

Expression of this gene is also upregulated in TNF-alpha/LPS-treated neutrophils (CT=25.8) compared to resting neutrophils (CT=27.7). Thus, the gene product may increase activation of these inflammatory cells and therapeutic modulation of the activity of this gene may be of benefit in the treatment of Crohn's disease, ulcerative colitis, multiple sclerosis, chronic obstructive pulmonary disease, asthma, emphysema, rheumatoid arthritis, lupus erythematosus, or psoriasis.

This gene is also highly expressed in a cluster of treated and untreated samples derived from the NCI-H292 cell line, a human airway epithelial cell line that produces mucins. Mucus overproduction is an important feature of bronchial asthma and chronic obstructive pulmonary disease. The transcript is also expressed at lower but still significant levels in small airway epithelium treated with IL-1 beta and TNF-alpha. The expression of the transcript in this mucoepidermoid cell line that is often used as a model for airway epithelium (NCI-H292 cells) suggests that this transcript may be important in the proliferation or activation of airway epithelium. Therefore, therapeutics designed with the protein encoded by the transcript may reduce or eliminate symptoms caused by inflammation in lung epithelia in chronic obstructive pulmonary disease, asthma, allergy, and emphysema.

This gene encodes a splice variant of diamine acetyltransferase, also known as spermidine/spermine N(1)-acetyltransferase (SPD/SPM acetyltransferase). Diamine acetyltransferase is a rate-limiting enzyme in the catabolic pathway of polyamine metabolism. It catalyzes the N(1)-acetylation of spermidine and spermine and, by the successive activity of polyamine oxidase, spermine can be converted to spermidine and spermidine to putrescine. The role of spermine in inflammation was reviewed by Zhang et al. [Crit Care Med. April 2000;28(4 Suppl):N60-6, PMID: 10807317]. Regenerating tissues produce higher levels of spermine, and injured or dying cells release spermine into the extracellular milieu, so that tissue levels increase significantly at inflammatory sites of infection or injury. Recent research has focused on delineating the significance of spermine accumulation in the inflammatory process. The discovery that spermine is a negative regulator of macrophage activation provided a mechanism by which spermine influences the biology of inflammation. Mechanistic studies indicate that spermine is incorporated into macrophages and restrains the innate immune response.

Panel 5D Summary: Ag4716 This gene is expressed at moderate to high levels in the majority of metabolic tissues on this panel, with highest expression in a placenta sample from a diabetic patient (CTs=23-25).

Spermine has been demonstrated to enhance insulin receptor binding in a dose dependent manner [Pedersen et al., Mol Cell Endocrinol., April 1989;62(2): 161-6]. Thus, it was proposed that polyamines may act as intracellular or intercellular (autocrine) regulators to modulate insulin binding. It has also been shown that the insulin-like effects elicited by polyamines in fat cells (e.g. enhancement of glucose transport and inhibition of cAMP-mediated lipolysis) are dependent on H2O2 production (Livingston et al., J. Biol. Chem., Jan. 25, 1977;252(2):560-2). Inhibiting polyamine catabolism through an inhibitor of this rate-limiting enzyme may abolish the insulin-like antilipolytic effects of polyamines. Therefore, therapeutic inhibition of the activity of this gene using small molecule drugs may be beneficial in the treatment of obesity.

S. CG164449-01: Granzyme H Precursor-Like Protein.

Expression of gene CG164449-01 was assessed using the primer-probe set Ag7846, described in Table SA. Results of the RTQ-PCR runs are shown in Table SB.

TABLE SA

Probe Name Ag7846

| | | | |
|---|---|---|---|
| | | | SEQ ID |
| Primers | Length | Start Position | No |

| | | | | |
|---|---|---|---|---|
| Forward | 5′-aggccaagtggaccacag-3′ | 18 | 376 | 422 |
| Probe | TET-5′-ctacctagcagcaaggcccagg-3′-TAMRA | 22 | 411 | 423 |
| Reverse | 5′-ggctacgtccttacacacga-3′ | 20 | 451 | 424 |

TABLE SB
General_screening_panel_v1.7

| | Rel. |
|---|---|
| | Exp. (%) |
| | Ag7846, |
| | Run |
| Tissue Name | 318010064 |
| Adipose | 6.9 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 0.0 |
| Prostate ca. (bone met) PC-3 | 0.0 |
| Prostate ca. DU145 | 0.0 |
| Prostate pool | 0.3 |
| Uterus pool | 0.0 |
| Ovarian ca. OVCAR-3 | 0.0 |
| Ovarian ca. (ascites) SK-OV-3 | 0.0 |
| Ovarian ca. OVCAR-4 | 0.0 |
| Ovarian ca. OVCAR-5 | 1.5 |
| Ovarian ca. IGROV-1 | 0.0 |
| Ovarian ca. OVCAR-8 | 0.0 |
| Ovary | 3.7 |
| Breast ca. MCF-7 | 0.0 |
| Breast ca. MDA-MB-231 | 0.0 |
| Breast ca. BT-549 | 0.0 |
| Breast ca. T47D | 0.0 |
| Breast pool | 0.0 |
| Trachea | 13.9 |
| Lung | 100.0 |
| Fetal Lung | 2.6 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. NCI-H23 | 0.0 |
| Lung ca. NCI-H460 | 0.0 |
| Lung ca. HOP-62 | 2.8 |
| Lung ca. NCI-H522 | 0.0 |
| Lung ca. DMS-114 | 0.0 |
| Liver | 0.5 |
| Fetal Liver | 0.4 |
| Kidney pool | 3.3 |
| Fetal Kidney | 1.7 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 4.7 |
| Gastric ca. (liver met.) NCI-N87 | 0.0 |
| Stomach | 0.0 |
| Colon ca. SW-948 | 0.2 |
| Colon ca. SW480 | 0.0 |
| Colon ca. (SW480 met) SW620 | 0.0 |
| Colon ca. HT29 | 0.0 |
| Colon ca. HCT-116 | 0.0 |
| Colon cancer tissue | 0.1 |
| Colon ca. SW1116 | 0.0 |
| Colon ca. Colo-205 | 1.9 |
| Colon ca. SW-48 | 0.0 |
| Colon | 0.7 |
| Small Intestine | 0.0 |
| Fetal Heart | 0.0 |
| Heart | 2.4 |
| Lymph Node pool 1 | 0.0 |
| Lymph Node pool 2 | 21.3 |
| Fetal Skeletal Muscle | 0.3 |
| Skeletal Muscle pool | 0.0 |
| Skeletal Muscle | 0.0 |
| Spleen | 7.1 |
| Thymus | 0.5 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 0.3 |
| Brain (Cerebellum) | 0.3 |
| Brain (Fetal) | 0.0 |
| Brain (Hippocampus) | 0.6 |
| Cerebral Cortex pool | 0.2 |
| Brain (Substantia nigra) | 0.0 |
| Brain (Thalamus) | 0.1 |
| Brain (Whole) | 0.7 |
| Spinal Cord | 0.2 |
| Adrenal Gland | 6.2 |
| Pituitary Gland | 1.4 |
| Salivary Gland | 4.0 |
| Thyroid | 6.4 |
| Pancreatic ca. PANC-1 | 0.0 |
| Pancreas pool | 0.0 |

General_screening_panel_v1.7 Summary: Ag7846 Highest expression of this gene is detected in lung (CT=30.2). Therefore, expression of this gene may be used to distinguish lung from other samples in the is panel. Furthermore, therapeutic modulation of this gene or its protein product may be useful in the treatment of lung related disorders.

In addition, low expression of this gene is also seen in Lymph Node pool 2 and trachea. Therefore, expression of this gene may be used as marker to detect lymph node and trachea and also therapeutic modulation of this gene may be useful in the treatment of lymph node related or trachea related disorders.

T. CG54007-04 and CG54007-06: Carboxypeptidase X Precursor-Like Protein.

Expression of gene CG54007-04 and CG54007-06 were assessed using the primer-probe sets Ag874, Ag86, Ag544 and Ag5121, described in Tables TA, TB, TC and TD. Results of the RTQ-PCR runs are shown in Tables TE, TF, TG, TH, TI, TJ, TK and TL. Please note that probe-primer set Ag5121 is specific for Cg CG54007-04. Also, please note that CG54007-06 represents a full length physical clone.

TABLE TA

Probe Name Ag874

| | | | | |
|---|---|---|---|---|
| | | | | SEQ ID |
| Primers | Sequenes | Length | Start Position | No |

| | | | | |
|---|---|---|---|---|
| Forward | 5′-acagggcaggaactctgtct-3′ | 20 | 567 | 425 |
| Probe | TET-5′-tgactgggtcacatcatacaaggtcca-3′-TAMRA | 27 | 594 | 426 |
| Reverse | 5′-gtccgactgtcattgctgaa-3′ | 20 | 622 | 427 |

TABLE TB

Probe Name Ag86

| | | | | |
|---|---|---|---|---|
| | | | | SEQ ID |
| Primers | Sequenes | Length | Start Position | No |

| | | | | |
|---|---|---|---|---|
| Forward | 5′-gtctggagtccctgcgagttt-3′ | 21 | 356 | 428 |
| Probe | TET-5′-cttgaggcatccagcagccagtcc-3′-TAMRA | 24 | 388 | 429 |
| Reverse | 5′-cggtgtggtccaagaccaa-3′ | 19 | 413 | 430 |

TABLE TC

Probe Name Ag544

| | | | | |
|---|---|---|---|---|
| | | | | SEQ ID |
| Primers | Seqences | Length | Start Position | No |

| | | | | |
|---|---|---|---|---|
| Forward | 5′-cctgcgtcgggatcctct-3′ | 18 | 859 | 431 |
| Probe | TET-5′-cctctagactttcagcatcacaattacaaggcc-3′-TAMRA | 33 | 880 | 432 |
| Reverse | 5′-cctgcttcatcagcttcctca-3′ | 21 | 914 | 433 |

TABLE TD

Probe Name Ag5121

| | | | | |
|---|---|---|---|---|
| | | | | SEQ ID |
| Primers | Sequencs | Length | Start Position | No |

| | | | | |
|---|---|---|---|---|
| Forward | 5′-acccattcgacatggtga-3′ | 18 | 1517 | 434 |
| Probe | TET-5′-ctaccattcagtgacacggaactgtcg-3′-TAMRA | 27 | 1551 | 435 |
| Reverse | 5′-ggccctcttcaaaggtga-3′ | 18 | 1580 | 436 |

TABLE TE
AI_comprehensive_panel_v1.0

| | Rel. | Rel. |
|---|---|---|
| | Exp. (%) | Exp. (%) |
| | Ag5121, | Ag874, |
| | Run | Run |
| Tissue Name | 275481195 | 220260120 |
| 110967 COPD-F | 7.0 | 20.0 |
| 110980 COPD-F | 0.0 | 0.9 |
| 110968 COPD-M | 11.0 | 19.1 |
| 110977 COPD-M | 1.2 | 0.0 |
| 110989 Emphysema-F | 31.0 | 88.9 |
| 110992 Emphysema-F | 20.0 | 39.5 |
| 110993 Emphysema-F | 6.4 | 14.8 |
| 110994 Emphysema-F | 2.0 | 5.7 |
| 110995 Emphysema-F | 28.7 | 42.3 |
| 110996 Emphysema-F | 15.6 | 20.6 |
| 110997 Asthma-M | 0.0 | 1.5 |
| 111001 Asthma-F | 2.7 | 9.5 |
| 111002 Asthma-F | 12.2 | 31.2 |
| 111003 Atopic Asthma-F | 20.9 | 59.0 |
| 111004 Atopic Asthma-F | 43.8 | 79.0 |
| 111005 Atopic Asthma-F | 34.4 | 53.2 |
| 111006 Atopic Asthma-F | 10.5 | 11.9 |
| 111417 Allergy-M | 9.9 | 31.0 |
| 112347 Allergy-M | 0.0 | 0.4 |
| 112349 Normal Lung-F | 0.0 | 0.7 |
| 112357 Normal Lung-F | 20.0 | 8.7 |
| 112354 Normal Lung-M | 3.7 | 3.8 |
| 112374 Crohns-F | 36.1 | 7.6 |
| 112389 Match Control Crohns-F | 0.0 | 1.9 |
| 112375 Crohns-F | 38.2 | 23.7 |
| 112732 Match Control Crohns-F | 0.0 | 0.4 |
| 112725 Crohns-M | 2.8 | 1.2 |
| 112387 Match Control Crohns-M | 3.8 | 16.7 |
| 112378 Crohns-M | 0.0 | 0.8 |
| 112390 Match Control Crohns-M | 12.4 | 22.8 |
| 112726 Crohns-M | 27.4 | 16.7 |
| 112731 Match Control Crohns-M | 8.0 | 5.5 |
| 112380 Ulcer Col-F | 25.3 | 21.3 |
| 112734 Match Control Ulcer Col-F | 0.0 | 0.4 |
| 112384 Ulcer Col-F | 19.9 | 15.0 |
| 112737 Match Control Ulcer Col-F | 10.8 | 5.0 |
| 112386 Ulcer Col-F | 0.0 | 7.6 |
| 112738 Match Control Ulcer Col-F | 0.0 | 1.3 |
| 112381 Ulcer Col-M | 0.0 | 3.0 |
| 112735 Match Control Ulcer Col-M | 0.0 | 8.5 |
| 112382 Ulcer Col-M | 0.0 | 3.3 |
| 112394 Match Control Ulcer Col-M | 0.0 | 2.3 |
| 112383 Ulcer Col-M | 100.0 | 100.0 |
| 112736 Match Control Ulcer Col-M | 3.5 | 3.1 |
| 112423 Psoriasis-F | 3.4 | 3.7 |
| 112427 Match Control Psoriasis-F | 10.5 | 10.4 |
| 112418 Psoriasis-M | 1.7 | 4.6 |
| 112723 Match Control Psoriasis-M | 60.7 | 40.9 |
| 112419 Psoriasis-M | 0.0 | 14.4 |
| 112424 Match Control Psoriasis-M | 3.7 | 6.9 |
| 112420 Psoriasis-M | 27.7 | 77.9 |
| 112425 Match Control Psoriasis-M | 4.2 | 26.1 |
| 104689 (MF) OA Bone-Backus | 12.6 | 13.0 |
| 104690 (MF) Adj “Normal” Bone-Backus | 0.0 | 0.2 |
| 104691 (MF) OA Synovium-Backus | 0.0 | 0.4 |
| 104692 (BA) OA Cartilage-Backus | 0.0 | 0.1 |
| 104694 (BA) OA Bone-Backus | 6.1 | 8.5 |
| 104695 (BA) Adj “Normal” Bone-Backus | 0.0 | 0.5 |
| 104696 (BA) OA Synovium-Backus | 3.0 | 3.9 |
| 104700 (SS) OA Bone-Backus | 1.2 | 1.8 |
| 104701 (SS) Adj “Normal” Bone-Backus | 0.0 | 7.4 |
| 104702 (SS) OA Synovium-Backus | 5.8 | 7.6 |
| 117093 OA Cartilage Rep7 | 4.9 | 59.5 |
| 112672 OA Bone5 | 3.3 | 35.1 |
| 112673 OA Synovium5 | 4.7 | 16.4 |
| 112674 OA Synovial Fluid cells5 | 2.0 | 15.8 |
| 117100 OA Cartilage Rep14 | 10.2 | 11.4 |
| 112756 OA Bone9 | 0.0 | 1.2 |
| 112757 OA Synovium9 | 0.0 | 0.1 |
| 112758 OA Synovial Fluid Cells9 | 3.7 | 4.5 |
| 117125 RA Cartilage Rep2 | 2.1 | 9.9 |
| 113492 Bone2 RA | 1.0 | 0.2 |
| 113493 Synovium2 RA | 0.0 | 0.0 |
| 113494 Syn Fluid Cells RA | 0.0 | 0.1 |
| 113499 Cartilage4 RA | 0.0 | 0.2 |
| 113500 Bone4 RA | 0.0 | 0.4 |
| 113501 Synovium4 RA | 0.0 | 0.4 |
| 113502 Syn Fluid Cells4 RA | 0.0 | 0.2 |
| 113495 Cartilage3 RA | 0.0 | 0.2 |
| 113496 Bone3 RA | 0.0 | 0.1 |
| 113497 Synovium3 RA | 0.0 | 0.0 |
| 113498 Syn Fluid Cells3 RA | 0.0 | 0.1 |
| 117106 Normal Cartilage Rep20 | 3.5 | 15.6 |
| 113663 Bone3 Normal | 0.0 | 0.5 |
| 113664 Synovium3 Normal | 0.0 | 0.8 |
| 113665 Syn Fluid Cells3 Normal | 0.0 | 0.3 |
| 117107 Normal Cartilage Rep22 | 0.0 | 8.1 |
| 113667 Bone4 Normal | 4.2 | 23.7 |
| 113668 Synovium4 Normal | 4.5 | 27.4 |
| 113669 Syn Fluid Cells4 Normal | 17.3 | 37.4 |

TABLE TF
CNS_neurodegeneration_v1.0

| | Rel. |
|---|---|
| | Exp. (%) |
| | Ag874, |
| | Run |
| Tissue Name | 271695187 |
| AD 1 Hippo | 10.1 |
| AD 2 Hippo | 54.0 |
| AD 3 Hippo | 9.3 |
| AD 4 Hippo | 13.4 |
| AD 5 hippo | 25.5 |
| AD 6 Hippo | 100.0 |
| Control 2 Hippo | 18.8 |
| Control 4 Hippo | 28.3 |
| Control (Path) 3 Hippo | 12.9 |
| AD 1 Temporal Ctx | 5.0 |
| AD 2 Temporal Ctx | 32.3 |
| AD 3 Temporal Ctx | 0.0 |
| AD 4 Temporal Ctx | 7.8 |
| AD 5 Inf Temporal Ctx | 8.3 |
| AD 5 Sup Temporal Ctx | 39.8 |
| AD 6 Inf Temporal Ctx | 27.9 |
| AD 6 Sup Temporal Ctx | 28.1 |
| Control 1 Temporal Ctx | 68.3 |
| Control 2 Temporal Ctx | 28.5 |
| Control 3 Temporal Ctx | 24.7 |
| Control 4 Temporal Ctx | 13.5 |
| Control (Path) 1 Temporal Ctx | 31.6 |
| Control (Path) 2 Temporal Ctx | 30.1 |
| Control (Path) 3 Temporal Ctx | 4.6 |
| Control (Path) 4 Temporal Ctx | 9.9 |
| AD 1 Occipital Ctx | 12.2 |
| AD 2 Occipital Ctx (Missing) | 0.0 |
| AD 3 Occipital Ctx | 1.8 |
| AD 4 Occipital Ctx | 13.6 |
| AD 5 Occipital Ctx | 36.6 |
| AD 6 Occipital Ctx | 55.9 |
| Control 1 Occipital Ctx | 40.1 |
| Control 2 Occipital Ctx | 27.0 |
| Control 3 Occipital Ctx | 12.2 |
| Control 4 Occipital Ctx | 4.6 |
| Control (Path) 1 Occipital Ctx | 31.6 |
| Control (Path) 2 Occipital Ctx | 0.0 |
| Control (Path) 3 Occipital Ctx | 0.0 |
| Control (Path) 4 Occipital Ctx | 7.9 |
| Control 1 Parietal Ctx | 48.3 |
| Control 2 Parietal Ctx | 23.0 |
| Control 3 Parietal Ctx | 17.8 |
| Control (Path) 1 Parietal Ctx | 34.6 |
| Control (Path) 2 Parietal Ctx | 49.0 |
| Control (Path) 3 Parietal Ctx | 0.0 |
| Control (Path) 4 Parietal Ctx | 18.6 |

TABLE TG
Panel 1

| | Rel. | Rel. |
|---|---|---|
| | Exp. (%) | Exp. (%) |
| | Ag86, | Ag86, |
| | Run | Run |
| Tissue Name | 87584059 | 87589776 |
| Endothelial cells | 0.2 | 0.0 |
| Endothelial cells (treated) | 0.9 | 0.0 |
| Pancreas | 1.1 | 0.1 |
| Pancreatic ca. CAPAN2 | 0.0 | 0.0 |
| Adrenal gland | 3.4 | 3.2 |
| Thyroid | 22.1 | 27.7 |
| Salivary gland | 3.0 | 1.9 |
| Pituitary gland | 16.2 | 27.7 |
| Brain (fetal) | 4.1 | 4.8 |
| Brain (whole) | 0.8 | 0.0 |
| Brain (amygdala) | 0.7 | 0.1 |
| Brain (cerebellum) | 1.0 | 0.1 |
| Brain (hippocampus) | 2.0 | 0.2 |
| Brain (substantia nigra) | 0.2 | 0.0 |
| Brain (thalamus) | 0.3 | 0.0 |
| Brain (hypothalamus) | 1.8 | 0.9 |
| Spinal cord | 4.4 | 6.1 |
| glio/astro U87-MG | 0.0 | 0.0 |
| glio/astro U-118-MG | 0.0 | 0.0 |
| astrocytoma SW1783 | 0.1 | 0.0 |
| neuro*; met SK-N-AS | 6.7 | 17.2 |
| astrocytoma SF-539 | 0.1 | 0.0 |
| astrocytoma SNB-75 | 0.1 | 0.0 |
| glioma SNB-19 | 0.1 | 0.0 |
| glioma U251 | 0.0 | 0.0 |
| glioma SF-295 | 0.0 | 0.0 |
| Heart | 2.5 | 2.4 |
| Skeletal muscle | 0.1 | 0.0 |
| Bone marrow | 3.9 | 0.0 |
| Thymus | 14.6 | 24.7 |
| Spleen | 0.5 | 0.1 |
| Lymph node | 3.5 | 5.2 |
| Colon (ascending) | 0.9 | 0.6 |
| Stomach | 3.0 | 3.8 |
| Small intestine | 1.8 | 1.8 |
| Colon ca. SW480 | 0.6 | 0.0 |
| Colon ca.* SW620 (SW480 met) | 0.0 | 0.0 |
| Colon ca. HT29 | 0.1 | 0.0 |
| Colon ca. HCT-116 | 0.0 | 0.0 |
| Colon ca. CaCo-2 | 0.0 | 0.0 |
| Colon ca. HCT-15 | 0.2 | 0.1 |
| Colon ca. HCC-2998 | 0.0 | 0.0 |
| Gastric ca.* (liver met) NCI-N87 | 0.0 | 0.0 |
| Bladder | 4.2 | 15.5 |
| Trachea | 2.5 | 4.6 |
| Kidney | 3.5 | 4.2 |
| Kidney (fetal) | 90.8 | 92.7 |
| Renal ca. 786-0 | 0.0 | 0.0 |
| Renal ca. A498 | 0.1 | 0.0 |
| Renal ca. RXF 393 | 0.0 | 0.0 |
| Renal ca. ACHN | 0.0 | 0.0 |
| Renal ca. UO-31 | 0.1 | 0.0 |
| Renal ca. TK-10 | 0.0 | 0.0 |
| Liver | 0.7 | 0.1 |
| Liver (fetal) | 3.0 | 3.0 |
| Liver ca. (hepatoblast) HepG2 | 0.0 | 0.0 |
| Lung | 0.5 | 2.8 |
| Lung (fetal) | 19.2 | 17.3 |
| Lung ca. (small cell) LX-1 | 0.0 | 0.0 |
| Lung ca. (small cell) NCI-H69 | 0.3 | 0.0 |
| Lung ca. (s. cell var.) SHP-77 | 0.0 | 0.0 |
| Lung ca. (large cell) NCI-H460 | 0.0 | 0.0 |
| Lung ca. (non-sm. cell) A549 | 0.1 | 0.0 |
| Lung ca. (non-s. cell) NCI-H23 | 1.8 | 2.4 |
| Lung ca. (non-s. cell) HOP-62 | 1.8 | 1.2 |
| Lung ca. (non-s. cl) NCI-H522 | 0.1 | 0.0 |
| Lung ca. (squam.) SW 900 | 0.0 | 0.0 |
| Lung ca. (squam.) NCI-H596 | 0.5 | 0.1 |
| Mammary gland | 46.3 | 55.9 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.1 | 0.0 |
| Breast ca. BT-549 | 0.0 | 11.4 |
| Breast ca. MDA-N | 0.1 | 0.0 |
| Ovary | 100.0 | 100.0 |
| Ovarian ca. OVCAR-3 | 0.2 | 0.0 |
| Ovarian ca. OVCAR-4 | 0.0 | 0.0 |
| Ovarian ca. OVCAR-5 | 0.2 | 0.0 |
| Ovarian ca. OVCAR-8 | 1.7 | 0.8 |
| Ovarian ca. IGROV-1 | 0.0 | 0.0 |
| Ovarian ca. (ascites) SK-OV-3 | 0.1 | 0.0 |
| Uterus | 4.2 | 8.4 |
| Placenta | 55.1 | 64.2 |
| Prostate | 4.7 | 8.5 |
| Prostate ca.* (bone met) PC-3 | 0.0 | 0.0 |
| Testis | 13.2 | 15.4 |
| Melanoma Hs688(A).T | 0.3 | 0.0 |
| Melanoma* (met) Hs688(B).T | 0.0 | 0.0 |
| Melanoma UACC-62 | 0.0 | 0.0 |
| Melanoma M14 | 0.1 | 0.0 |
| Melanoma LOX IMVI | 0.0 | 0.0 |
| Melanoma* (met) SK-MEL-5 | 0.1 | 0.0 |
| Melanoma SK-MEL-28 | 0.0 | 0.0 |

TABLE TH
Panel 1.1

| | Rel. |
|---|---|
| | Exp. (%) |
| | Ag544, |
| | Run |
| Tissue Name | 111164655 |
| Adrenal gland | 4.8 |
| Bladder | 24.3 |
| Brain (amygdala) | 0.3 |
| Brain (cerebellum) | 0.3 |
| Brain (hippocampus) | 0.6 |
| Brain (substantia nigra) | 0.9 |
| Brain (thalamus) | 0.2 |
| Cerebral Cortex | 0.1 |
| Brain (fetal) | 3.8 |
| Brain (whole) | 0.3 |
| glio/astro U-118-MG | 0.0 |
| astrocytoma SF-539 | 0.0 |
| astrocytoma SNB-75 | 0.0 |
| astrocytoma SW1783 | 0.1 |
| glioma U251 | 0.0 |
| glioma SF-295 | 0.0 |
| glioma SNB-19 | 0.0 |
| glio/astro U87-MG | 0.0 |
| neuro*; met SK-N-AS | 26.2 |
| Mammary gland | 39.5 |
| Breast ca. BT-549 | 4.0 |
| Breast ca. MDA-N | 0.0 |
| Breast ca.* (pl. ef) T47D | 0.0 |
| Breast ca.* (pl. ef) MCF-7 | 0.0 |
| Breast ca.* (pl. ef) MDA-MB-231 | 0.0 |
| Small intestine | 2.5 |
| Colorectal | 1.0 |
| Colon ca. HT29 | 0.0 |
| Colon ca. CaCo-2 | 0.0 |
| Colon ca. HCT-15 | 0.0 |
| Colon ca. HCT-116 | 0.0 |
| Colon ca. HCC-2998 | 0.0 |
| Colon ca. SW480 | 0.9 |
| Colon ca.* SW620 (SW480 met) | 0.0 |
| Stomach | 3.3 |
| Gastric ca. (liver met) NCI-N87 | 0.0 |
| Heart | 11.4 |
| Skeletal muscle (Fetal) | 18.2 |
| Skeletal muscle | 0.9 |
| Endothelial cells | 2.0 |
| Heart (Fetal) | 17.7 |
| Kidney | 5.4 |
| Kidney (fetal) | 55.5 |
| Renal ca. 786-0 | 0.0 |
| Renal ca. A498 | 0.0 |
| Renal ca. ACHN | 0.0 |
| Renal ca. TK-10 | 0.0 |
| Renal ca. UO-31 | 0.0 |
| Renal ca. RXF 393 | 0.0 |
| Liver | 2.3 |
| Liver (fetal) | 1.2 |
| Liver ca. (hepatoblast) HepG2 | 0.0 |
| Lung | 0.7 |
| Lung (fetal) | 15.1 |
| Lung ca. (non-s. cell) HOP-62 | 15.9 |
| Lung ca. (large cell) NCI-H460 | 0.0 |
| Lung ca. (non-s. cell) NCI-H23 | 4.0 |
| Lung ca. (non-s. cl) NCI-H522 | 0.4 |
| Lung ca. (non-sm. cell) A549 | 0.0 |
| Lung ca. (s. cell var.) SHP-77 | 0.0 |
| Lung ca. (small cell) LX-1 | 0.0 |
| Lung ca. (small cell) NCI-H69 | 0.0 |
| Lung ca. (squam.) SW 900 | 0.0 |
| Lung ca. (squam.) NCI-H596 | 0.4 |
| Lymph node | 2.5 |
| Spleen | 0.1 |
| Thymus | 4.5 |
| Ovary | 100.0 |
| Ovarian ca. IGROV-1 | 0.0 |
| Ovarian ca. OVCAR-3 | 0.5 |
| Ovarian ca. OVCAR-4 | 0.0 |
| Ovarian ca. OVCAR-5 | 0.0 |
| Ovarian ca. OVCAR-8 | 1.9 |
| Ovarian ca.* (ascites) SK-OV-3 | 0.0 |
| Pancreas | 2.5 |
| Pancreatic ca. CAPAN 2 | 0.0 |
| Pituitary gland | 2.5 |
| Placenta | 29.5 |
| Prostate | 4.5 |
| Prostate ca.* (bone met) PC-3 | 0.0 |
| Salivary gland | 8.5 |
| Trachea | 1.7 |
| Spinal cord | 4.0 |
| Testis | 1.8 |
| Thyroid | 22.7 |
| Uterus | 17.4 |
| Melanoma M14 | 0.0 |
| Melanoma LOX IMVI | 0.0 |
| Melanoma UACC-62 | 0.0 |
| Melanoma SK-MEL-28 | 0.0 |
| Melanoma* (met) SK-MEL-5 | 0.0 |
| Melanoma Hs688(A).T | 0.4 |
| Melanoma* (met) Hs688(B).T | 0.1 |

TABLE TI
Panel 1.3D

| | Rel. | Rel. |
|---|---|---|
| | Exp. (%) | Exp. (%) |
| | Ag544, | Ag874, |
| | Run | Run |
| Tissue Name | 165702011 | 152932054 |
| Liver adenocarcinoma | 0.0 | 0.0 |
| Pancreas | 0.3 | 0.1 |
| Pancreatic ca. CAPAN2 | 0.0 | 0.0 |
| Adrenal gland | 3.2 | 1.1 |
| Thyroid | 16.3 | 4.2 |
| Salivary gland | 3.6 | 0.5 |
| Pituitary gland | 2.5 | 0.3 |
| Brain (fetal) | 5.1 | 0.7 |
| Brain (whole) | 1.2 | 0.2 |
| Brain (amygdala) | 1.1 | 0.2 |
| Brain (cerebellum) | 0.3 | 0.0 |
| Brain (hippocampus) | 1.3 | 0.8 |
| Brain (substantia nigra) | 1.2 | 0.1 |
| Brain (thalamus) | 0.3 | 0.0 |
| Cerebral Cortex | 0.4 | 0.3 |
| Spinal cord | 6.7 | 0.7 |
| glio/astro U87-MG | 0.0 | 0.0 |
| glio/astro U-118-MG | 0.0 | 0.0 |
| astrocytoma SW1783 | 0.8 | 0.1 |
| neuro*; met SK-N-AS | 43.2 | 7.1 |
| astrocytoma SF-539 | 0.0 | 0.0 |
| astrocytoma SNB-75 | 0.7 | 0.1 |
| glioma SNB-19 | 0.0 | 0.0 |
| glioma U251 | 0.0 | 0.0 |
| glioma SF-295 | 0.0 | 0.0 |
| Heart (fetal) | 11.3 | 10.2 |
| Heart | 1.8 | 0.3 |
| Skeletal muscle (fetal) | 26.4 | 43.2 |
| Skeletal muscle | 1.2 | 0.1 |
| Bone marrow | 10.6 | 1.4 |
| Thymus | 5.1 | 1.3 |
| Spleen | 1.2 | 0.2 |
| Lymph node | 8.4 | 1.0 |
| Colorectal | 0.5 | 0.5 |
| Stomach | 4.2 | 0.7 |
| Small intestine | 4.7 | 0.6 |
| Colon ca. SW480 | 1.4 | 0.8 |
| Colon ca.* SW620 (SW480 met) | 0.0 | 0.0 |
| Colon ca. HT29 | 0.0 | 0.1 |
| Colon ca. HCT-116 | 0.0 | 0.0 |
| Colon ca. CaCo-2 | 0.0 | 0.0 |
| Colon ca. tissue (ODO3866) | 17.2 | 2.3 |
| Colon ca. HCC-2998 | 0.0 | 0.0 |
| Gastric ca.* (liver met) NCI-N87 | 0.2 | 0.0 |
| Bladder | 5.8 | 0.7 |
| Trachea | 3.0 | 0.7 |
| Kidney | 0.9 | 0.2 |
| Kidney (fetal) | 44.1 | 9.8 |
| Renal ca. 786-0 | 0.0 | 0.0 |
| Renal ca. A498 | 0.7 | 0.0 |
| Renal ca. RXF 393 | 0.8 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 9.1 | 1.9 |
| Liver ca. (hepatoblast) HepG2 | 0.0 | 0.0 |
| Lung | 1.7 | 0.2 |
| Lung (fetal) | 37.6 | 9.3 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 0.0 |
| Lung ca. (large cell) NCI-H460 | 0.2 | 0.0 |
| Lung ca. (non-sm. cell) A549 | 0.0 | 0.0 |
| Lung ca. (non-s. cell) NCI-H23 | 2.6 | 1.4 |
| Lung ca. (non-s. cell) HOP-62 | 2.1 | 0.6 |
| Lung ca. (non-s. cl) NCI-H522 | 0.0 | 0.1 |
| Lung ca. (squam.) SW 900 | 0.0 | 0.0 |
| Lung ca. (squam.) NCI-H596 | 0.7 | 0.1 |
| Mammary gland | 50.7 | 13.1 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 12.2 | 1.7 |
| Breast ca. MDA-N | 0.0 | 0.0 |
| Ovary | 100.0 | 100.0 |
| Ovarian ca. OVCAR-3 | 1.6 | 0.1 |
| Ovarian ca. OVCAR-4 | 0.0 | 0.0 |
| Ovarian ca. OVCAR-5 | 0.0 | 0.0 |
| Ovarian ca. OVCAR-8 | 3.6 | 0.3 |
| Ovarian ca. IGROV-1 | 0.0 | 0.0 |
| Ovarian ca.* (ascites) SK-OV-3 | 0.0 | 0.0 |
| Uterus | 80.1 | 9.5 |
| Placenta | 28.5 | 7.6 |
| Prostate | 5.3 | 1.3 |
| Prostate ca.* (bone met) PC-3 | 0.0 | 0.0 |
| Testis | 6.2 | 1.1 |
| Melanoma Hs688(A).T | 0.7 | 0.1 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 32.1 | 7.6 |

TABLE TJ
Panel 2D

| | Rel. |
|---|---|
| | Exp. (%) |
| | Ag874, |
| | Run |
| Tissue Name | 152932207 |
| Normal Colon | 16.0 |
| CC Well to Mod Diff (ODO3866) | 8.1 |
| CC Margin (ODO3866) | 0.6 |
| CC Gr. 2 rectosigmoid (ODO3868) | 3.7 |
| CC Margin (ODO3868) | 1.3 |
| CC Mod Diff (ODO3920) | 2.3 |
| CC Margin (ODO3920) | 1.7 |
| CC Gr. 2 ascend colon (ODO3921) | 9.9 |
| CC Margin (ODO3921) | 2.4 |
| CC from Partial Hepatectomy (ODO4309) Mets | 2.8 |
| Liver Margin (ODO4309) | 0.3 |
| Colon mets to lung (OD04451-01) | 2.4 |
| Lung Margin (OD04451-02) | 0.2 |
| Normal Prostate 6546-1 | 10.0 |
| Prostate Cancer (OD04410) | 9.7 |
| Prostate Margin (OD04410) | 10.2 |
| Prostate Cancer (OD04720-01) | 5.4 |
| Prostate Margin (OD04720-02) | 15.7 |
| Normal Lung 061010 | 3.2 |
| Lung Met to Muscle (ODO4286) | 1.8 |
| Muscle Margin (ODO4286) | 8.1 |
| Lung Malignant Cancer (OD03126) | 6.9 |
| Lung Margin (OD03126) | 1.0 |
| Lung Cancer (OD04404) | 18.2 |
| Lung Margin (OD04404) | 12.4 |
| Lung Cancer (OD04565) | 7.1 |
| Lung Margin (OD04565) | 0.2 |
| Lung Cancer (OD04237-01) | 6.2 |
| Lung Margin (OD04237-02) | 2.8 |
| Ocular Mel Met to Liver (ODO4310) | 0.0 |
| Liver Margin (ODO4310) | 0.2 |
| Melanoma Mets to Lung (OD04321) | 4.6 |
| Lung Margin (OD04321) | 0.4 |
| Normal Kidney | 5.1 |
| Kidney Ca, Nuclear grade 2 (OD04338) | 1.0 |
| Kidney Margin (OD04338) | 1.8 |
| Kidney Ca Nuclear grade 1/2 (OD04339) | 0.1 |
| Kidney Margin (OD04339) | 2.6 |
| Kidney Ca, Clear cell type (OD04340) | 0.4 |
| Kidney Margin (OD04340) | 4.5 |
| Kidney Ca, Nuclear grade 3 (OD04348) | 10.9 |
| Kidney Margin (OD04348) | 3.2 |
| Kidney Cancer (OD04622-01) | 6.3 |
| Kidney Margin (OD04622-03) | 1.3 |
| Kidney Cancer (OD04450-01) | 0.0 |
| Kidney Margin (OD04450-03) | 3.5 |
| Kidney Cancer 8120607 | 1.8 |
| Kidney Margin 8120608 | 0.9 |
| Kidney Cancer 8120613 | 0.3 |
| Kidney Margin 8120614 | 2.8 |
| Kidney Cancer 9010320 | 32.5 |
| Kidney Margin 9010321 | 5.2 |
| Normal Uterus | 13.0 |
| Uterus Cancer 064011 | 11.7 |
| Normal Thyroid | 18.7 |
| Thyroid Cancer 064010 | 0.9 |
| Thyroid Cancer A302152 | 1.4 |
| Thyroid Margin A302153 | 20.0 |
| Normal Breast | 22.4 |
| Breast Cancer (OD04566) | 1.7 |
| Breast Cancer (OD04590-01) | 8.4 |
| Breast Cancer Mets (OD04590-03) | 5.6 |
| Breast Cancer Metastasis (OD04655-05) | 2.1 |
| Breast Cancer 064006 | 10.2 |
| Breast Cancer 1024 | 38.4 |
| Breast Cancer 9100266 | 13.4 |
| Breast Margin 9100265 | 36.3 |
| Breast Cancer A209073 | 21.6 |
| Breast Margin A209073 | 16.7 |
| Normal Liver | 0.0 |
| Liver Cancer 064003 | 0.3 |
| Liver Cancer 1025 | 0.2 |
| Liver Cancer 1026 | 4.9 |
| Liver Cancer 6004-T | 0.1 |
| Liver Tissue 6004-N | 1.5 |
| Liver Cancer 6005-T | 5.2 |
| Liver Tissue 6005-N | 0.4 |
| Normal Bladder | 12.5 |
| Bladder Cancer 1023 | 8.9 |
| Bladder Cancer A302173 | 3.4 |
| Bladder Cancer (OD04718-01) | 13.0 |
| Bladder Normal Adjacent (OD04718-03) | 87.7 |
| Normal Ovary | 100.0 |
| Ovarian Cancer 064008 | 61.6 |
| Ovarian Cancer (OD04768-07) | 1.2 |
| Ovary Margin (OD04768-08) | 24.3 |
| Normal Stomach | 1.4 |
| Gastric Cancer 9060358 | 3.2 |
| Stomach Margin 9060359 | 3.1 |
| Gastric Cancer 9060395 | 7.0 |
| Stomach Margin 9060394 | 11.2 |
| Gastric Cancer 9060397 | 12.0 |
| Stomach Margin 9060396 | 1.1 |
| Gastric Cancer 064005 | 7.3 |

TABLE TK
Panel 4D

| | Rel. | Rel. | Rel. |
|---|---|---|---|
| | Exp. (%) | Exp. (%) | Exp. (%) |
| | Ag544, | Ag874, | Ag874, |
| | Run | Run | Run |
| Tissue Name | 145644930 | 138642062 | 144170545 |
| Secondary Th1 act | 0.0 | 0.7 | 1.1 |
| Secondary Th2 act | 0.5 | 25.2 | 0.4 |
| Secondary Tr1 act | 0.3 | 1.5 | 1.7 |
| Secondary Th1 rest | 0.0 | 0.0 | 0.0 |
| Secondary Th2 rest | 0.0 | 3.7 | 0.5 |
| Secondary Tr1 rest | 0.0 | 0.0 | 0.0 |
| Primary Th1 act | 0.9 | 1.0 | 0.6 |
| Primary Th2 act | 1.1 | 2.5 | 2.8 |
| Primary Tr1 act | 3.4 | 2.5 | 0.0 |
| Primary Th1 rest | 5.8 | 6.4 | 5.2 |
| Primary Th2 rest | 2.5 | 6.5 | 2.9 |
| Primary Tr1 rest | 0.7 | 1.0 | 1.4 |
| CD45RA CD4 lymphocyte | 4.2 | 6.8 | 7.6 |
| act | | | |
| CD45RO CD4 lymphocyte | 2.3 | 2.4 | 4.1 |
| act | | | |
| CD8 lymphocyte act | 1.2 | 1.3 | 0.7 |
| Secondary CD8 | 5.4 | 6.7 | 11.2 |
| lymphocyte rest | | | |
| Secondary CD8 | 2.5 | 3.2 | 1.7 |
| lymphocyte act | | | |
| CD4 lymphocyte none | 0.0 | 0.0 | 0.0 |
| 2ry Th1/Th2/Tr1_anti- | 1.0 | 0.7 | 0.0 |
| CD95 CH11 | | | |
| LAK cells rest | 0.8 | 0.0 | 0.4 |
| LAK cells IL-2 | 0.0 | 3.0 | 1.2 |
| LAK cells IL-2 + IL-12 | 6.6 | 15.9 | 10.5 |
| LAK cells IL-2 + IFN | 5.1 | 7.6 | 3.8 |
| gamma | | | |
| LAK cells IL-2 + IL-18 | 7.3 | 8.4 | 4.3 |
| LAK cells PMA/ionomycin | 3.9 | 3.4 | 3.1 |
| NK Cells IL-2 rest | 2.1 | 0.5 | 0.5 |
| Two Way MLR 3 day | 0.6 | 0.3 | 1.2 |
| Two Way MLR 5 day | 0.7 | 2.5 | 1.1 |
| Two Way MLR 7 day | 10.3 | 8.4 | 9.9 |
| PBMC rest | 0.5 | 0.0 | 0.0 |
| PBMC PWM | 15.9 | 25.9 | 14.8 |
| PBMC PHA-L | 32.5 | 44.4 | 26.1 |
| Ramos (B cell) none | 0.0 | 0.0 | 0.0 |
| Ramos (B cell) | 0.0 | 0.0 | 0.0 |
| ionomycin | | | |
| B lymphocytes PWM | 20.3 | 33.2 | 13.7 |
| B lymphocytes CD40L | 20.4 | 34.2 | 12.6 |
| and IL-4 | | | |
| EOL-1 dbcAMP | 0.6 | 0.3 | 1.7 |
| EOL-1 dbcAMP | 1.4 | 2.1 | 0.5 |
| PMA/ionomycin | | | |
| Dendritic cells none | 0.0 | 0.0 | 0.0 |
| Dendritic cells LPS | 0.9 | 0.6 | 0.3 |
| Dendritic cells anti- | 0.0 | 0.3 | 0.0 |
| CD40 | | | |
| Monocytes rest | 0.0 | 0.0 | 0.4 |
| Monocytes LPS | 1.9 | 4.3 | 6.0 |
| Macrophages rest | 1.0 | 0.6 | 0.0 |
| Macrophages LPS | 4.9 | 5.6 | 2.9 |
| HUVEC none | 5.9 | 6.2 | 5.2 |
| HUVEC starved | 10.9 | 14.1 | 10.9 |
| HUVEC IL-1beta | 3.3 | 6.5 | 4.2 |
| HUVEC IFN gamma | 20.7 | 18.6 | 18.9 |
| HUVEC TNF alpha + IFN | 2.0 | 2.2 | 2.1 |
| gamma | | | |
| HUVEC TNF alpha + IL4 | 7.1 | 4.3 | 4.6 |
| HUVEC IL-11 | 7.7 | 4.3 | 3.6 |
| Lung Microvascular | 4.6 | 1.5 | 2.5 |
| EC none | | | |
| Lung Microvascular | 1.0 | 3.0 | 1.7 |
| EC TNFalpha + IL- | | | |
| 1beta | | | |
| Microvascular Dermal | 0.4 | 1.5 | 0.3 |
| EC none | | | |
| Microsvasular Dermal | 1.5 | 1.0 | 3.6 |
| EC TNFalpha + IL- | | | |
| 1beta | | | |
| Bronchial epithelium | 0.0 | 0.2 | 0.0 |
| TNFalpha + IL1beta | | | |
| Small airway | 0.0 | 0.0 | 0.0 |
| epithelium none | | | |
| Small airway | 0.6 | 0.8 | 0.0 |
| epithelium TNFalpha + | | | |
| IL-1beta | | | |
| Coronery artery SMC | 1.8 | 0.8 | 1.5 |
| rest | | | |
| Coronery artery SMC | 1.0 | 0.3 | 0.7 |
| TNFalpha + IL-1beta | | | |
| Astrocytes rest | 7.1 | 10.2 | 9.9 |
| Astrocytes TNFalpha + | 3.2 | 2.7 | 5.8 |
| IL-1beta | | | |
| KU-812 (Basophil) rest | 16.0 | 24.5 | 23.7 |
| KU-812 (Basophil) | 30.4 | 35.6 | 44.4 |
| PMA/ionomycin | | | |
| CCD1106 (Keratinocytes) | 0.0 | 0.0 | 0.0 |
| none | | | |
| CCD1106 (Keratinocytes) | 0.0 | 1.8 | 0.0 |
| TNFalpha + IL-1beta | | | |
| Liver cirrhosis | 10.9 | 8.8 | 6.8 |
| Lupus kidney | 8.9 | 7.7 | 4.1 |
| NCI-H292 none | 0.0 | 0.0 | 0.0 |
| NCI-H292 IL-4 | 0.7 | 0.0 | 0.0 |
| NCI-H292 IL-9 | 0.0 | 0.0 | 0.0 |
| NCI-H292 IL-13 | 0.0 | 0.0 | 0.0 |
| NCI-H292 IFN gamma | 0.0 | 0.3 | 0.4 |
| HPAEC none | 2.3 | 2.8 | 1.6 |
| HPAEC TNF alpha + | 4.7 | 10.0 | 7.5 |
| IL-1 beta | | | |
| Lung fibroblast none | 0.0 | 0.6 | 1.3 |
| Lung fibroblast TNF | 1.7 | 4.5 | 2.4 |
| alpha + IL-1 beta | | | |
| Lung fibroblast IL-4 | 0.6 | 0.3 | 0.6 |
| Lung fibroblast IL-9 | 0.0 | 0.0 | 0.4 |
| Lung fibroblast IL-13 | 1.4 | 1.8 | 0.0 |
| Lung fibroblast IFN | 2.7 | 2.5 | 1.7 |
| gamma | | | |
| Dermal fibroblast | 20.9 | 27.2 | 13.7 |
| CCD1070 rest | | | |
| Dermal fibroblast | 7.6 | 10.9 | 10.7 |
| CCD1070 TNF alpha | | | |
| Dermal fibroblast | 20.6 | 15.4 | 20.0 |
| CCD1070 IL-1 beta | | | |
| Dermal fibroblast IFN | 47.3 | 48.6 | 35.6 |
| gamma | | | |
| Dermal fibroblast IL-4 | 40.3 | 39.5 | 43.2 |
| IBD Colitis 2 | 1.0 | 1.2 | 1.2 |
| IBD Crohn's | 4.9 | 4.3 | 4.5 |
| Colon | 2.9 | 7.0 | 5.9 |
| Lung | 100.0 | 100.0 | 100.0 |
| Thymus | 24.0 | 15.2 | 18.0 |
| Kidney | 27.2 | 53.2 | 42.6 |

TABLE TL
Panel 5D

| | Rel. | Rel. |
|---|---|---|
| | Exp. (%) | Exp. (%) |
| | Ag544, | Ag874, |
| | Run | Run |
| Tissue Name | 247855022 | 166667617 |
| 97457_Patient-02go_adipose | 100.0 | 100.0 |
| 97476_Patient-07sk_skeletal | 10.2 | 12.7 |
| muscle | | |
| 97477_Patient-07ut_uterus | 6.0 | 4.4 |
| 97478_Patient-07pl_placenta | 8.9 | 4.4 |
| 97481_Patient-08sk_skeletal | 6.4 | 2.7 |
| muscle | | |
| 97482_Patient-08ut_uterus | 3.7 | 2.4 |
| 97483_Patient-08pl_placenta | 2.0 | 4.7 |
| 97486_Patient-09sk_skeletal | 0.1 | 0.2 |
| muscle | | |
| 97487_Patient-09ut_uterus | 6.3 | 3.0 |
| 97488_Patient-09pl_placenta | 3.4 | 1.7 |
| 97492_Patient-10ut_uterus | 9.9 | 5.7 |
| 97493_Patient-10pl_placenta | 6.4 | 10.8 |
| 97495_Patient-11go_adipose | 0.0 | 12.9 |
| 97496_Patient-11sk_skeletal | 0.1 | 0.1 |
| muscle | | |
| 97497_Patient-11ut_uterus | 2.9 | 1.9 |
| 97498_Patient-11pl_placenta | 0.4 | 1.8 |
| 97500_Patient-12go_adipose | 41.5 | 26.8 |
| 97501_Patient-12sk_skeletal | 0.7 | 0.4 |
| muscle | | |
| 97502_Patient-12ut_uterus | 1.8 | 3.4 |
| 97503_Patient-12pl_placenta | 1.0 | 1.4 |
| 94721_Donor 2 U - A_Mesenchymal | 0.0 | 0.0 |
| Stem Cells | | |
| 94722_Donor 2 U - B_Mesenchymal | 0.0 | 0.2 |
| Stem Cells | | |
| 94723_Donor 2 U - C_Mesenchymal | 0.0 | 0.0 |
| Stem Cells | | |
| 94709_Donor 2 AM - A_adipose | 0.0 | 0.0 |
| 94710_Donor 2 AM - B_adipose | 0.0 | 0.0 |
| 94711_Donor 2 AM - C_adipose | 0.0 | 0.0 |
| 94712_Donor 2 AD - A_adipose | 0.0 | 0.0 |
| 94713_Donor 2 AD - B_adipose | 0.0 | 0.0 |
| 94714_Donor 2 AD - C_adipose | 0.0 | 0.0 |
| 94742_Donor 3 U - A_Mesenchymal | 0.0 | 0.0 |
| Stem Cells | | |
| 94743_Donor 3 U - B_Mesenchymal | 0.0 | 0.0 |
| Stem Cells | | |
| 94730_Donor 3 AM - A_adipose | 0.0 | 0.2 |
| 94731_Donor 3 AM - B_adipose | 0.0 | 0.0 |
| 94732_Donor 3 AM - C_adipose | 0.0 | 0.0 |
| 94733_Donor 3 AD - A_adipose | 0.0 | 0.0 |
| 94734_Donor 3 AD - B_adipose | 0.1 | 0.0 |
| 94735_Donor 3 AD - C_adipose | 0.1 | 0.0 |
| 77138_Liver_HepG2untreated | 0.1 | 0.0 |
| 73556_Heart_Cardiac stromal | 0.0 | 0.3 |
| cells (primary) | | |
| 81735_Small Intestine | 0.7 | 0.8 |
| 72409_Kidney_Proximal Convoluted | 0.2 | 0.3 |
| Tubule | | |
| 82685_Small intestine_Duodenum | 0.0 | 0.3 |
| 90650_Adrenal_Adrenocortical | 0.3 | 0.7 |
| adenoma | | |
| 72410_Kidney_HRCE | 0.2 | 0.2 |
| 72411_Kidney_HRE | 2.2 | 2.1 |
| 73139_Uterus_Uterine smooth | 0.5 | 0.4 |
| muscle cells | | |

AI_comprehensive panel_v1.0 Summary: Ag5121/Ag874 Two experiments with different probe-primer sets are in good agreement. Highest expression of this gene is detected in ulcerative colitis sample (CT=28-33). Interestingly, expression of this gene is higher in colitis compared the matched control sample. Therefore, expression of this may be used as marker for ulcerative colitis and therapeutic modulation of this gene may be useful in the treatment of ulcerative colitis.

In addition, moderate to low expression of this gene is also seen in in samples derived from normal and orthoarthitis bone, cartilage, synovium and synovial fluid samples, RA cartilage REP2, from normal lung, COPD lung, emphysema, atopic asthma, asthma, allergy, Crohn's disease (normal matched control and diseased), ulcerative colitis (normal matched control and diseased), and psoriasis (normal matched control and diseased). Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis

CNS_neurodegeneration_v1.0 Summary: Ag874 Low expression of this gene is restricted to hippocampus from an Alzheimer's patient (CT=33.99). Therefore, therapeutic modulation of this gene may be useful in the treatment of seizure.

Panel 1 Summary: Ag86 Two experiments with same probe-primer sets are in good agreement. Highest expression of this gene is detected in ovary (CT=21-24). High expression of this gene is detected in normal tissues including testis, placenta, prostate, uterus, mammary gland, kidney, trachea, bladder, brain, and tissues with metabolic/endocrine functions including pancreas, heart and gastrointestinal tract.

This gene codes for metallocarboxypeptidase CPX-1. It is a member of a family of enzymatically inactive carboxypeptidases including CPX-2 and AEBP-1/ACLP [1]. These enzymes lack several putative active site residues but retain binding activity to substrate proteins. They also contain a domain related to discoidin. Carboxypeptidases can act as binding proteins, perhaps blocking the function of other carboxypeptidases or mediating cell-cell interactions. Carboxypeptidases have been shown to play important roles in metabolic disorders including obesity and diabetes. Several of these enzymes are involved in propeptide processing of prohormone peptides to active hormones. Mutation of carboxypeptidase E in mice results in the fat/fat phenotype, demonstrating hyperproinsulinemia, and late onset diabetes and obesity [2]. ACLP has been shown to associate with the extracellular matrix and deficiency of ACLP results in impaired wound healing and abdominal wall development [3]. In addition, ACLP protein and mRNA are downregulated during adipocyte differentiation [4]. Therefore, CPX-1 encoded by this gene can be used as potential protein therapeutic for obesity.

Interestingly, this gene is expressed at much higher levels in fetal (CTs=21-29.4) when compared to adult liver, lung and kidney (CTs=26.8-32.5). This observation suggests that expression of this gene can be used to distinguish fetal from adult liver. In addition, the relative overexpression of this gene in fetal tissue suggests that the protein product may enhance liver, lung, and kidney growth or development in the fetus and thus may also act in a regenerative capacity in the adult. Therefore, therapeutic modulation of the protein encoded by this gene could be useful in treatment of liver, lung and kidney related diseases.

Moderate to low expression of this gene is also seen in number of cell lines derived from ovarian, breast, lung, and brain cancers. Therefore, therapeutic modulation of this gene may be useful in the treatment of, breast, lung, and brain cancers.

Fricker L D, Leiter E H. Peptides, enzymes and obesity: new insights from a ‘dead’ enzyme. Trends Biochem Sci October 1999;24(10):390-3; Naggert J K, Fricker L D, Varlamov O, Nishina P M, Rouille Y, Steiner D F, Carroll R J, Paigen B J, Leiter E H. Hyperproinsulinaemia in obese fat/fat mice associated with a carboxypeptidase E mutation which reduces enzyme activity. Nat Genet June 1995;10(2):135-42; Layne M D, Yet S F, Maemura K, Hsieh C M, Bernfield M, Perrella M A, Lee M E. Impaired abdominal wall development and deficient wound healing in mice lacking aortic carboxypeptidase-like protein. Mol Cell Biol August 2001;21(15):5256-61; Gagnon A, Abaiian K J, Crapper T, Layne M D, Sorisky A. Down-Regulation of Aortic Carboxypeptidase-Like Protein during the Early Phase of 3T3-L1 Adipogenesis. Endocrinology July 2002;143(7):2478-85.

Panel 1.1 Summary: Ag544 Highest expression of this gene is detected in ovary (CT=22.5). This gene shows high expression in normal tissues, which correlates with the expression seen in panel 1. Please see panel 1 for further discussion of this gene.

Panel 1.3D Summary: Ag544/Ag874 Two experiments with different probe-primer sets are in good agreement. Highest expression of this gene is detected in ovary (CTs=27-29). This gene shows significant expression in normal tissues and number of cancer cell lines, which correlates with the expression seen in panel 1. Please see panel 1 for further discussion on the utility of this gene.

Panel 2D Summary: Ag874 Highest expression of this gene is seen in normal ovary (CT=27.9). Moderate to low expression of this gene is seen in normal and cancer samples derived from stomach, ovary, bladder, liver, breast, thyroid, uterus, kidney, lung, prostate and colon. Therefore, therapeutic modulation of this gene or its protein product may be useful in the treatment of stomach, ovary, bladder, liver, breast, thyroid, uterus, kidney, lung, prostate and colon cancers.

Panel 4D Summary: Ag544/Ag874 Three experiments with two different probe-primer sets are in good agreement. Highest expression of this gene is detected in lung (CTs=30-31.4). Moderate to low expression of this gene is also seen in resting and activated dermal fibroblasts, basophils, HUVEC, activated PBMC and B lymphocytes and normal tissues represented by thymus and kidney. Therefore, therapeutic modulation of this gene or its protein product may be useful in the treatment of asthma, allergies, inflammatory bowel disease, lupus erythematosus, psoriasis, rheumatoid arthritis, and osteoarthritis.

Panel 5D Summary: Ag544/Ag874 Two experiments with two different probe-primer sets are in good agreement. Highest expression of this gene is detected in adipose from a diabetic patient not on insulin (CTs=28-29). Moderate to low expression of this gene is also seen in adipose, skeletal muscle, uterus, and placenta from diabetic anc non-diabetic patients. Therefore, therapeutic modulation of this gene through the use of small molecule drug could be useful in the treatment of obesity and diabetes including Type II diabetes.

U. CG55078-01 and CG55078-03: Serine Carboxypeptidase 1 Precursor-Like Protein.

Expression of gene CG55078-01 and CG55078-01 was assessed using the primer-probe set Ag3450, described in Table UA. Results of the RTQ-PCR runs are shown in Tables UB, UC, UD, UE and UF.

TABLE UA

Probe Name Ag3450

| | | | | |
|---|---|---|---|---|
| | | | | SEQ ID |
| Primers | Sequence | Length | Start Position | No |

| | | | | |
|---|---|---|---|---|
| Forward | 5′-ctttggaaacatctgcttttgt-3′ | 22 | 1256 | 437 |
| Probe | TET-5′-tcctacaagaaccttgctttctactgg-3′-TAMRA | 27 | 1282 | 438 |
| Reverse | 5′-ccatatgaccagctttcagaat-3′ | 22 | 1309 | 439 |

TABLE UB
CNS_neurodegeneration_v1.0

| | Rel. |
|---|---|
| | Exp. (%) |
| | Ag3450, |
| | Run |
| Tissue Name | 269217277 |
| AD 1 Hippo | 24.0 |
| AD 2 Hippo | 70.2 |
| AD 3 Hippo | 9.4 |
| AD 4 Hippo | 23.0 |
| AD 5 hippo | 79.0 |
| AD 6 Hippo | 95.3 |
| Control 2 Hippo | 62.9 |
| Control 4 Hippo | 34.9 |
| Control (Path) 3 Hippo | 13.2 |
| AD 1 Temporal Ctx | 21.9 |
| AD 2 Temporal Ctx | 53.6 |
| AD 3 Temporal Ctx | 1.3 |
| AD 4 Temporal Ctx | 32.1 |
| AD 5 Inf Temporal Ctx | 95.9 |
| AD 5 Sup Temporal Ctx | 74.2 |
| AD 6 Inf Temporal Ctx | 41.5 |
| AD 6 Sup Temporal Ctx | 67.4 |
| Control 1 Temporal Ctx | 8.4 |
| Control 2 Temporal Ctx | 55.5 |
| Control 3 Temporal Ctx | 20.9 |
| Control 4 Temporal Ctx | 15.6 |
| Control (Path) 1 Temporal Ctx | 85.3 |
| Control (Path) 2 Temporal Ctx | 92.0 |
| Control (Path) 3 Temporal Ctx | 8.3 |
| Control (Path) 4 Temporal Ctx | 47.0 |
| AD 1 Occipital Ctx | 27.0 |
| AD 2 Occipital Ctx (Missing) | 0.0 |
| AD 3 Occipital Ctx | 7.1 |
| AD 4 Occipital Ctx | 27.4 |
| AD 5 Occipital Ctx | 18.8 |
| AD 6 Occipital Ctx | 57.4 |
| Control 1 Occipital Ctx | 5.7 |
| Control 2 Occipital Ctx | 47.3 |
| Control 3 Occipital Ctx | 24.1 |
| Control 4 Occipital Ctx | 84.1 |
| Control (Path) 1 Occipital Ctx | 84.1 |
| Control (Path) 2 Occipital Ctx | 14.2 |
| Control (Path) 3 Occipital Ctx | 2.7 |
| Control (Path) 4 Occipital Ctx | 45.1 |
| Control 1 Parietal Ctx | 11.2 |
| Control 2 Parietal Ctx | 63.7 |
| Control 3 Parietal Ctx | 27.5 |
| Control (Path) 1 Parietal Ctx | 100.0 |
| Control (Path) 2 Parietal Ctx | 30.8 |
| Control (Path) 3 Parietal Ctx | 5.7 |
| Control (Path) 4 Parietal Ctx | 54.3 |

TABLE UC
Panel 1.3D

| | Rel. |
|---|---|
| | Exp (%) |
| | Ag3450, |
| | Run |
| Tissue Name | 167819116 |
| Liver adenocarcinoma | 11.3 |
| Pancreas | 6.7 |
| Pancreatic ca. CAPAN 2 | 6.8 |
| Adrenal gland | 58.2 |
| Thyroid | 64.2 |
| Salivary gland | 14.2 |
| Pituitary gland | 22.1 |
| Brain (fetal) | 3.4 |
| Brain (whole) | 28.9 |
| Brain (amygdala) | 31.2 |
| Brain (cerebellum) | 11.1 |
| Brain (hippocampus) | 23.7 |
| Brain (substantia nigra) | 46.7 |
| Brain (thalamus) | 13.1 |
| Cerebral Cortex | 12.8 |
| Spinal cord | 23.2 |
| glio/astro U87-MG | 19.8 |
| glio/astro U-118-MG | 24.5 |
| astrocytoma SW1783 | 17.7 |
| neuro*; met SK-N-AS | 15.4 |
| astrocytoma SF-539 | 32.1 |
| astrocytoma SNB-75 | 77.4 |
| glioma SNB-19 | 7.2 |
| glioma U251 | 52.1 |
| glioma SF-295 | 71.7 |
| Heart (fetal) | 8.7 |
| Heart | 27.7 |
| Skeletal muscle (fetal) | 3.0 |
| Skeletal muscle | 18.6 |
| Bone marrow | 23.5 |
| Thymus | 10.4 |
| Spleen | 22.4 |
| Lymph node | 22.5 |
| Colorectal | 5.6 |
| Stomach | 16.8 |
| Small intestine | 14.4 |
| Colon ca. SW480 | 8.1 |
| Colon ca.* SW620 (SW480 met) | 23.0 |
| Colon ca. HT29 | 1.7 |
| Colon ca. HCT-116 | 6.0 |
| Colon ca. CaCo-2 | 25.3 |
| Colon ca. tissue (ODO3866) | 9.4 |
| Colon ca. HCC-2998 | 8.6 |
| Gastric ca.* (liver met) NCI-N87 | 8.8 |
| Bladder | 11.7 |
| Trachea | 22.1 |
| Kidney | 93.3 |
| Kidney (fetal) | 89.5 |
| Renal ca. 786-0 | 7.2 |
| Renal ca. A498 | 42.3 |
| Renal ca. RXF 393 | 19.2 |
| Renal ca. ACHN | 6.8 |
| Renal ca. UO-31 | 9.0 |
| Renal ca. TK-10 | 2.1 |
| Liver | 18.9 |
| Liver (fetal) | 9.8 |
| Liver ca. (hepatoblast) HepG2 | 1.5 |
| Lung | 30.4 |
| Lung (fetal) | 36.9 |
| Lung ca. (small cell) LX-1 | 4.2 |
| Lung ca. (small cell) NCI-H69 | 6.0 |
| Lung ca. (s. cell var.) SHP-77 | 30.8 |
| Lung ca. (large cell) NCI-H460 | 6.1 |
| Lung ca. (non-sm. cell) A549 | 18.6 |
| Lung ca. (non-s. cell) NCI-H23 | 13.0 |
| Lung ca. (non-s. cell) HOP-62 | 42.0 |
| Lung ca. (non-s. cl) NCI-H522 | 14.6 |
| Lung ca. (squam.) SW 900 | 68.8 |
| Lung ca. (squam.) NCI-H596 | 22.2 |
| Mammary gland | 100.0 |
| Breast ca.* (pl. ef) MCF-7 | 14.2 |
| Breast ca.* (pl. ef) MDA-MB-231 | 6.6 |
| Breast ca.* (pl. ef) T47D | 74.2 |
| Breast ca. BT-549 | 13.5 |
| Breast ca. MDA-N | 9.7 |
| Ovary | 29.9 |
| Ovarian ca. OVCAR-3 | 20.3 |
| Ovarian ca. OVCAR-4 | 26.1 |
| Ovarian ca. OVCAR-5 | 98.6 |
| Ovarian ca. OVCAR-8 | 3.3 |
| Ovarian ca. IGROV-1 | 1.1 |
| Ovarian ca.* (ascites) SK-OV-3 | 19.1 |
| Uterus | 26.1 |
| Placenta | 1.1 |
| Prostate | 34.6 |
| Prostate ca.* (bone met) PC-3 | 22.4 |
| Testis | 4.2 |
| Melanoma Hs688(A).T | 11.0 |
| Melanoma* (met) Hs688(B).T | 13.4 |
| Melanoma UACC-62 | 20.0 |
| Melanoma M14 | 12.0 |
| Melanoma LOX IMVI | 10.4 |
| Melanoma* (met) SK-MEL-5 | 22.5 |
| Adipose | 53.6 |

TABLE UP
Panel 4.1D

| | Rel. |
|---|---|
| | Exp. (%) |
| | Ag3450, |
| | Run |
| Tissue Name | 268719219 |
| Secondary Th1 act | 8.7 |
| Secondary Th2 act | 7.0 |
| Secondary Tr1 act | 4.1 |
| Secondary Th1 rest | 0.9 |
| Secondary Th2 rest | 2.5 |
| Secondary Tr1 rest | 1.6 |
| Primary Th1 act | 0.5 |
| Primary Th2 act | 4.2 |
| Primary Tr1 act | 3.0 |
| Primary Th1 rest | 0.0 |
| Primary Th2 rest | 0.4 |
| Primary Tr1 rest | 0.1 |
| CD45RA CD4 lymphocyte act | 7.3 |
| CD45RO CD4 lymphocyte act | 11.2 |
| CD8 lymphocyte act | 1.8 |
| Secondary CD8 lymphocyte rest | 4.5 |
| Secondary CD8 lymphocyte act | 3.8 |
| CD4 lymphocyte none | 0.6 |
| 2ry Th1/Th2/Tr1_anti-CD95 CH11 | 2.2 |
| LAK cells rest | 40.9 |
| LAK cells IL-2 | 4.5 |
| LAK cells IL-2 + IL-12 | 0.1 |
| LAK cells IL-2 + IFN gamma | 3.0 |
| LAK cells IL-2 + IL-18 | 1.6 |
| LAK cells PMA/ionomycin | 94.0 |
| NK Cells IL-2 rest | 12.9 |
| Two Way MLR 3 day | 23.8 |
| Two Way MLR 5 day | 6.2 |
| Two Way MLR 7 day | 7.7 |
| PBMC rest | 4.9 |
| PBMC PWM | 1.8 |
| PBMC PHA-L | 3.1 |
| Ramos (B cell) none | 4.9 |
| Ramos (B cell) ionomycin | 6.3 |
| B lymphocytes PWM | 3.2 |
| B lymphocytes CD40L and IL-4 | 24.0 |
| EOL-1 dbcAMP | 10.2 |
| EOL-1 dbcAMP PMA/ionomycin | 12.3 |
| Dendritic cells none | 65.5 |
| Dendritic cells LPS | 27.4 |
| Dendritic cells anti-CD40 | 27.4 |
| Monocytes rest | 31.0 |
| Monocytes LPS | 48.0 |
| Macrophages rest | 28.7 |
| Macrophages LPS | 58.6 |
| HUVEC none | 12.2 |
| HUVEC starved | 19.2 |
| HUVEC IL-1beta | 14.6 |
| HUVEC IFN gamma | 15.7 |
| HUVEC TNF alpha + IFN gamma | 5.1 |
| HUVEC TNF alpha + IL4 | 3.1 |
| HUVEC IL-11 | 3.0 |
| Lung Microvascular EC none | 24.7 |
| Lung Microvascular EC TNFalpha + IL-1beta | 8.1 |
| Microvascular Dermal EC none | 4.4 |
| Microsvasular Dermal EC TNFalpha + IL-1beta | 2.8 |
| Bronchial epithelium TNFalpha + IL1beta | 14.1 |
| Small airway epithelium none | 13.6 |
| Small airway epithelium TNFalpha + IL-1beta | 25.3 |
| Coronery artery SMC rest | 25.9 |
| Coronery artery SMC TNFalpha + IL-1beta | 15.4 |
| Astrocytes rest | 2.8 |
| Astrocytes TNFalpha + IL-1beta | 2.6 |
| KU-812 (Basophil) rest | 26.1 |
| KU-812 (Basophil) PMA/ionomycin | 16.3 |
| CCD1106 (Keratinocytes) none | 25.5 |
| CCD1106 (Keratinocytes) TNFalpha + IL-1beta | 8.3 |
| Liver cirrhosis | 4.3 |
| NCI-H292 none | 100.0 |
| NCI-H292 IL-4 | 84.1 |
| NCI-H292 IL-9 | 97.9 |
| NCI-H292 IL-13 | 82.4 |
| NCI-H292 IFN gamma | 25.9 |
| HPAEC none | 8.7 |
| HPAEC TNF alpha + IL-1 beta | 18.8 |
| Lung fibroblast none | 25.5 |
| Lung fibroblast TNF alpha + IL-1 beta | 32.3 |
| Lung fibroblast IL-4 | 13.3 |
| Lung fibroblast IL-9 | 28.5 |
| Lung fibroblast IL-13 | 6.7 |
| Lung fibroblast IFN gamma | 34.9 |
| Dermal fibroblast CCD1070 rest | 13.9 |
| Dermal fibroblast CCD1070 TNF alpha | 33.2 |
| Dermal fibroblast CCD1070 IL-1 beta | 9.6 |
| Dermal fibroblast IFN gamma | 44.8 |
| Dermal fibroblast IL-4 | 32.3 |
| Dermal Fibroblasts rest | 32.5 |
| Neutrophils TNFa + LPS | 3.4 |
| Neutrophils rest | 14.6 |
| Colon | 4.3 |
| Lung | 4.9 |
| Thymus | 1.2 |
| Kidney | 29.1 |

TABLE UE
Panel 5D

| | Rel. |
|---|---|
| | Exp. (%) |
| | Ag450, |
| | Run |
| Tissue Name | 168095531 |
| 97457_Patient-02go_adipose | 42.3 |
| 97476_Patient-07sk_skeletal muscle | 45.4 |
| 97477_Patient-07ut_uterus | 51.1 |
| 97478_Patient-07pl_placenta | 11.1 |
| 97481_Patient-08sk_skeletal muscle | 33.9 |
| 97482_Patient-08ut_uterus | 24.7 |
| 97483_Patient-08pl_placenta | 5.4 |
| 97486_Patient-09sk_skeletal muscle | 11.7 |
| 97487_Patient-09ut_uterus | 15.8 |
| 97488_Patient-09pl_placenta | 5.3 |
| 97492_Patient-10ut_uterus | 28.9 |
| 97493_Patient-10pl_placenta | 15.6 |
| 97495_Patient-11go_adipose | 17.2 |
| 97496_Patient-11sk_skeletal muscle | 13.3 |
| 97497_Patient-11ut_uterus | 51.8 |
| 97498_Patient-11pl_placenta | 6.3 |
| 97500_Patient-12go_adipose | 57.4 |
| 97501_Patient-12sk_skeletal muscle | 37.1 |
| 97502_Patient-12ut_uterus | 54.3 |
| 97503_Patient-12pl_placenta | 11.2 |
| 94721_Donor 2 U - A_Mesenchymal Stem Cells | 84.7 |
| 94722_Donor 2 U - B_Mesenchymal Stem Cells | 55.5 |
| 94723_Donor 2 U- C_Mesenchymal Stem Cells | 52.5 |
| 94709_Donor 2 AM - A_adipose | 77.9 |
| 94710_Donor 2 AM - B_adipose | 44.4 |
| 94711_Donor 2 AM - C_adipose | 37.4 |
| 94712_Donor 2 AD - A_adipose | 74.7 |
| 94713_Donor 2 AD - B_adipose | 83.5 |
| 94714_Donor 2 AD - C_adipose | 85.3 |
| 94742_Donor 3 U - A_Mesenchymal Stem Cells | 40.1 |
| 94743_Donor 3 U - B_Mesenchymal Stem Cells | 84.7 |
| 94730_Donor 3 AM - A_adipose | 85.3 |
| 94731_Donor 3 AM - B_adipose | 56.3 |
| 94732_Donor 3 AM - C_adipose | 54.7 |
| 94733_Donor 3 AD - A_adipose | 81.8 |
| 94734_Donor 3 AD - B_adipose | 52.9 |
| 94735_Donor 3 AD - C_adipose | 51.8 |
| 77138_Liver HepG2untreated | 9.9 |
| 73556_Heart_Cardiac stromal cells (primary) | 15.5 |
| 81735_Small Intestine | 91.4 |
| 72409_Kidney_Proximal Convoluted Tubule | 4.1 |
| 82685_Small intestine_Duodenum | 39.8 |
| 90650_Adrenal_Adrenocortical adenoma | 100.0 |
| 72410_Kidney_HRCE | 24.1 |
| 72411_Kidney_HRE | 17.3 |
| 73139_Uterus_Uterine smooth muscle cells | 37.4 |

TABLE UF
general_oncology_screening_panel_v2.4

| | Rel. | | Rel. |
|---|---|---|---|
| | Exp. (%) | | Exp. (%) |
| | Ag3450, | | Ag3450, |
| | Run | | Run |
| Tissue Name | 267145071 | Tissue Name | 267145071 |
| Colon cancer 1 | 16.8 | Bladder cancer NAT 2 | 2.3 |
| Colon cancer | 12.1 | Bladder cancer NAT 3 | 0.5 |
| NAT 1 | | | |
| Colon cancer 2 | 15.3 | Bladder cancer NAT 4 | 4.7 |
| Colon cancer | 8.5 | Prostate adenocarcinoma | 34.6 |
| NAT 2 | | 1 | |
| Colon cancer 3 | 26.2 | Prostate adenocarcinoma | 3.9 |
| | | 2 | |
| Colon cancer | 22.5 | Prostate adenocarcinoma | 27.2 |
| NAT 3 | | 3 | |
| Colon | 31.2 | Prostate adenocarcinoma | 12.1 |
| malignant | | 4 | |
| cancer 4 | | | |
| Colon normal | 5.6 | Prostate cancer NAT 5 | 17.9 |
| adjacent | | | |
| tissue 4 | | | |
| Lung cancer 1 | 20.7 | Prostate adenocarcinoma | 9.3 |
| | | 6 | |
| Lung NAT 1 | 4.0 | Prostate adenocarcinoma | 12.9 |
| | | 7 | |
| Lung cancer 2 | 59.0 | Prostate adenocarcinoma | 3.3 |
| | | 8 | |
| Lung NAT 2 | 5.6 | Prostate adenocarcinoma | 29.7 |
| | | 9 | |
| Squamous cell | 72.2 | Prostate cancer NAT 10 | 2.8 |
| carcinoma 3 | | | |
| Lung NAT 3 | 6.3 | Kidney cancer 1 | 27.4 |
| metastatic | 24.3 | KidneyNAT 1 | 15.9 |
| melanoma 1 | | | |
| Melanoma 2 | 12.2 | Kidney cancer 2 | 100.0 |
| Melanoma 3 | 16.3 | Kidney NAT 2 | 50.0 |
| metastatic | 42.0 | Kidney cancer 3 | 13.2 |
| melanoma 4 | | | |
| metastatic | 69.7 | Kidney NAT 3 | 10.8 |
| melanoma 5 | | | |
| Bladder cancer | 10.7 | Kidney cancer 4 | 12.4 |
| 1 | | | |
| Bladder cancer | 0.0 | Kidney NAT 4 | 25.0 |
| NAT 1 | | | |
| Bladder cancer | 11.4 | | |
| 2 | | | |

CNS_neurodegeneration_v1.0 Summary: Ag3450 This panel does not show differential expression of this gene in Alzheimer's disease. However, this profile confirms the expression of this gene at moderate levels in the brain. Please see Panel 1.3D for discussion of this gene in the central nervous system.

Panel 1.3D Summary: Ag3450 Highest expression of this gene is seen in mammary gland (CT=28). This gene is widely expressed in this panel, with moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag3450 Highest expression of this gene is seen in untreated NCI-H292 cells (CT=29.3). The gene is also expressed in a cluster of cytokine activated samples derived from the NCI-H292 cell line, a human airway epithelial cell line that produces mucins. Mucus overproduction is an important feature of bronchial asthma and chronic obstructive pulmonary disease samples. The transcript is also expressed at lower but still significant levels in small airway epithelium, bronchial epithelium, and lung microvascular endothelial cells. The expression of the transcript in this mucoepidermoid cell line that is often used as a model for airway epithelium (NCI-H292 cells) suggests that this transcript may be important in the proliferation or activation of airway epithelium. Therefore, therapeutics designed with the protein encoded by the transcript may reduce or eliminate symptoms caused by inflammation in lung epithelia in chronic obstructive pulmonary disease, asthma, allergy, and emphysema.

Panel 5D Summary: Ag3450 Panel 5I shows that the target is widely expressed in metabolic tissues, specifically in adipose, which is in line with the data from panel 1.3.

general oncology screening panel_v_{—}2.4 Summary: Ag3450 Highest expression is seen in a kidney cancer (CT=28). In addition, this gene is more highly expressed in lung cancer than in the corresponding normal adjacent tissue, with prominent expression also detected in melanoma and prostate cancers. Thus, expression of this gene could be used as a marker of these cancers. Furthemore, therapeutic modulation of the expression or function of this gene product may be useful in the treatment of lung cancer.

W. CG56149-03: Nardilysin 1-Like Protein.

Expression of gene CG56149-03 was assessed using the primer-probe sets Ag1672 and Ag1673, described in Tables WA and WB. Results of the RTQ-PCR runs are shown in Table WC.

TABLE WA

Probe Name Ag1672

| | | | |
|---|---|---|---|
| | | | SEQ ID |
| Primers | Length | Start Position | No |

| | | | | |
|---|---|---|---|---|
| Forward | 5′-gaccaaactttggccatttaa-3′ | 21 | 1364 | 446 |
| Probe | TET-5′-cggatccatttgacacaccagcattt-3′-TAMRA | 26 | 1385 | 447 |
| Reverse | 5′-gtgatggtcagagcatgaattt-3′ | 22 | 1442 | 448 |

TABLE WB

Probe Name Ag1673

| | | | |
|---|---|---|---|
| | | | SEQ ID |
| Primers | Length | Start Position | No |

| | | | | |
|---|---|---|---|---|
| Forward | 5′-gaccaaactttggccatttaa-3′ | 21 | 1364 | 449 |
| Probe | TET-5′-cggatccatttgacacaccagcattt-3′-TAMRA | 26 | 1385 | 450 |
| Reverse | 5′-gtgatggtcagagcatgaattt-3′ | 22 | 1442 | 451 |

TABLE WC
Panel 1.3D

| | Rel. | Rel. |
|---|---|---|
| | Exp. (%) | Exp. (%) |
| | Ag1672, | Ag1673, |
| | Run | Run |
| Tissue Name | 147227540 | 146581465 |
| Liver adenocarcinoma | 41.8 | 36.6 |
| Pancreas | 5.3 | 7.4 |
| Pancreatic ca. CAPAN 2 | 9.5 | 9.1 |
| Adrenal gland | 13.3 | 16.6 |
| Thyroid | 16.8 | 18.6 |
| Salivary gland | 12.3 | 10.7 |
| Pituitary gland | 24.5 | 31.6 |
| Brain (fetal) | 8.2 | 9.7 |
| Brain (whole) | 25.7 | 26.8 |
| Brain (amygdala) | 20.0 | 21.5 |
| Brain (cerebellum) | 8.7 | 9.2 |
| Brain (hippocampus) | 41.2 | 37.4 |
| Brain (substantia nigra) | 7.2 | 9.0 |
| Brain (thalamus) | 9.3 | 20.7 |
| Cerebral Cortex | 33.7 | 39.2 |
| Spinal cord | 15.5 | 19.2 |
| glio/astro U87-MG | 44.8 | 52.1 |
| glio/astro U-118-MG | 100.0 | 89.5 |
| astrocytoma SW1783 | 29.5 | 45.1 |
| neuro*; met SK-N-AS | 64.6 | 67.4 |
| astrocytoma SF-539 | 33.2 | 34.4 |
| astrocytoma SNB-75 | 84.7 | 80.7 |
| glioma SNB-19 | 30.1 | 43.2 |
| glioma U251 | 32.8 | 41.5 |
| glioma SF-295 | 35.8 | 43.5 |
| Heart (fetal) | 17.0 | 18.3 |
| Heart | 10.7 | 11.6 |
| Skeletal muscle (fetal) | 49.0 | 44.4 |
| Skeletal muscle | 55.1 | 57.8 |
| Bone marrow | 18.3 | 23.5 |
| Thymus | 21.3 | 21.0 |
| Spleen | 14.8 | 20.0 |
| Lymph node | 18.7 | 21.2 |
| Colorectal | 7.0 | 10.4 |
| Stomach | 25.9 | 28.9 |
| Small intestine | 13.5 | 15.7 |
| Colon ca. SW480 | 52.5 | 50.0 |
| Colon ca.* SW620(SW480 met) | 19.3 | 21.8 |
| Colon ca. HT29 | 21.9 | 33.2 |
| Colon ca. HCT-116 | 35.4 | 29.9 |
| Colon ca. CaCo-2 | 32.3 | 35.4 |
| Colon ca. tissue(ODO3866) | 25.7 | 29.3 |
| Colon ca. HCC-2998 | 44.1 | 44.8 |
| Gastric ca.* (liver met) NCI-N87 | 95.3 | 100.0 |
| Bladder | 9.9 | 11.8 |
| Trachea | 23.5 | 30.8 |
| Kidney | 5.6 | 3.8 |
| Kidney (fetal) | 12.2 | 14.9 |
| Renal ca. 786-0 | 18.2 | 18.3 |
| Renal ca. A498 | 47.6 | 55.1 |
| Renal ca. RXF 393 | 6.8 | 9.9 |
| Renal ca. ACHN | 37.1 | 41.2 |
| Renal ca. UO-31 | 37.1 | 39.0 |
| Renal ca. TK-10 | 27.7 | 43.8 |
| Liver | 0.0 | 5.6 |
| Liver (fetal) | 24.7 | 31.2 |
| Liver ca. (hepatoblast) HepG2 | 27.9 | 31.6 |
| Lung | 12.2 | 14.6 |
| Lung (fetal) | 32.3 | 32.3 |
| Lung ca. (small cell) LX-1 | 21.5 | 34.6 |
| Lung ca. (small cell) NCI-H69 | 29.5 | 35.6 |
| Lung ca. (s. cell var.) SHP-77 | 61.6 | 61.6 |
| Lung ca. (large cell) NCI-H460 | 29.9 | 33.9 |
| Lung ca. (non-sm. cell) A549 | 16.8 | 15.2 |
| Lung ca. (non-s. cell) NCI-H23 | 79.0 | 92.7 |
| Lung ca. (non-s. cell) HOP-62 | 36.6 | 41.2 |
| Lung ca. (non-s. cl) NCI-H522 | 30.8 | 37.9 |
| Lung ca. (squam.) SW900 | 15.7 | 19.5 |
| Lung ca. (squam.) NCI-H596 | 15.0 | 15.8 |
| Mammary gland | 27.5 | 40.6 |
| Breast ca.* (pl. ef) MCF-7 | 46.7 | 42.9 |
| Breast ca.* (pl. ef) MDA-MB-231 | 84.7 | 86.5 |
| Breast ca.* (pl. ef) T47D | 36.3 | 34.4 |
| Breast ca. BT-549 | 94.0 | 80.1 |
| Breast ca. MDA-N | 27.7 | 29.5 |
| Ovary | 9.6 | 11.2 |
| Ovarian ca. OVCAR-3 | 21.6 | 23.0 |
| Ovarian ca. OVCAR-4 | 9.3 | 9.2 |
| Ovarian ca. OVCAR-5 | 37.1 | 34.6 |
| Ovarian ca. OVCAR-8 | 45.4 | 44.8 |
| Ovarian ca. IGROV-1 | 13.2 | 16.4 |
| Ovarian ca.* (ascites) SK-OV-3 | 66.4 | 63.7 |
| Uterus | 17.3 | 18.6 |
| Placenta | 37.9 | 37.1 |
| Prostate | 9.3 | 11.5 |
| Prostate ca.* (bone met)PC-3 | 23.8 | 35.8 |
| Testis | 87.1 | 84.1 |
| Melanoma Hs688(A).T | 55.5 | 57.8 |
| Melanoma* (met) Hs688(B).T | 74.7 | 88.9 |
| Melanoma UACC-62 | 3.0 | 3.8 |
| Melanoma M14 | 7.4 | 11.9 |
| Melanoma LOX IMVI | 3.3 | 4.4 |
| Melanoma* (met) SK-MEL-5 | 13.4 | 18.8 |
| Adipose | 12.4 | 13.8 |

Panel 1.3D Summary: Ag1672/Ag1673 Two experiments with the same probe and primer set produce results that are in excellent agreement with highest expression of the CG56149-01 gene in a gastric cancer cell line (NCI-N87) or a brain cancer cell line (U-118-MG)(CTs=26-27). Thus, the expression of this gene could be used to distinguish these samples from other samples in the panel.

This gene encodes a protein that is homologous to nardilysin, an N-arginine (R) dibasic (NRD) convertase metalloendopeptidase of the M16 family, that specifically cleaves peptide substrates at the N-terminus of arginines in dibasic motifs in vitro. The peptidase M16 family is also known as the insulinase family and nardilysin is the closest homolog of the insulin degrading enzyme, insulinase. The ability of nardilysin to degrade insulin has not been proven. However, the high levels of expression in metabolic tissues in this panel, including adipose, fetal and adult skeletal muscle, pancreas, adrenal, thyroid and pituitary glands suggest that this gene product may have a profound effect on limiting the degradation of insulin in tissues relevant to type II diabetes (e.g. adipose, skeletal muscle).

There is also a significant level of difference between expression in adult(CTs=31-40) and fetal liver tissue(CTs=28), making this gene and/or gene-product a good candidate for distinguishing both forms. A putative role for this gene-product is in the post-translational processing of bioactive peptides from their inactive precursors.

This gene is also highly expressed in the testis. Nardilysis has been implicated in spermiogenesis. Thus, expression of this gene could be used as a marker for testis tissue. Furthermore, therapeutic modulation of the expression or function of this gene may be useful in the treatment of male reproductive disorders.

Hospital V, Chesneau V, Balogh A, Joulie C, Seidah N G, Cohen P, Prat A. N-arginine dibasic convertase (nardilysin) isoforms are soluble dibasic-specific metalloendopeptidases that localize in the cytoplasm and at the cell surface. Biochem J Jul. 15, 2000;349(Pt 2):587-97, PMID: 10880358; Hospital V, Prat A, Joulie C, Cherif D, Day R, Cohen P. Human and rat testis express two mRNA species encoding variants of NRD convertase, a metalloendopeptidase of the insulinase family. Biochem J Nov. 1, 1997;327 (Pt 3):773-9. PMID: 9581555; Chesneau V, Prat A, Segretain D, Hospital V, Dupaix A, Foulon T, Jegou B, Cohen P. NRD convertase: a putative processing endoprotease associated with the axoneme and the manchette in late spermatids. J Cell Sci November 1996;109 (Pt 11):273745, PMID: 8937991.

X. CG56216-01 and CG56216-02: SERCA3-Like Protein.

Expression of gene CG56216-01 and CG56216-02 was assessed using the primer-probe sets Ag1800 and Ag3265, described in Tables XA and XB. Results of the RTQ-PCR runs are shown in Tables XC, XD, XE, XF, XG, XH, XI, XJ, XK, XL and XM.

TABLE XA

Probe Name Ag1800

| | | | |
|---|---|---|---|
| | | | SEQ ID |
| Primers | Length | Start Position | No |

| | | | | |
|---|---|---|---|---|
| Forward | 5′-atcaagactcacatccctttcc-3′ | 22 | 4259 | 452 |
| Probe | TET-5′-cacatccaaagcccctcagcctg-3′-TAMRA | 23 | 4289 | 453 |
| Reverse | 5′-ctacagaacatggagcccatt-3′ | 21 | 4323 | 454 |

TABLE XB

Probe Name Ag3265

| | | | |
|---|---|---|---|
| | | | SEQ ID |
| Primers | Length | Start Position | No |

| | | | | |
|---|---|---|---|---|
| Forward | 5′-cccaaatcacgagtgcagct-3′ | 20 | 3344 | 455 |
| Probe | TET-5′-agcttgctcccccttgttcggaag-3′-TAMRA | 24 | 3366 | 456 |
| Reverse | 5′-agaggcaccagtcagtcaccaagtg-3′ | 21 | 3399 | 457 |

TABLE XC
CNS neurodegeneration v1.0

| | Rel. | Rel. |
|---|---|---|
| | Exp. (%) | Exp. (%) |
| | Ag1800, | Ag3265, |
| | Run | Run |
| Tissue Name | 207742286 | 210038341 |
| AD 1 Hippo | 20.0 | 8.1 |
| AD 2 Hippo | 17.2 | 11.0 |
| AD 3 Hippo | 0.0 | 0.0 |
| AD 4 Hippo | 39.0 | 15.8 |
| AD 5 Hippo | 55.9 | 11.4 |
| AD 6 Hippo | 94.0 | 12.4 |
| Control 2 Hippo | 37.4 | 10.3 |
| Control 4 Hippo | 40.9 | 35.4 |
| Control (Path) 3 Hippo | 65.1 | 18.8 |
| AD 1 Temporal Ctx | 10.4 | 0.0 |
| AD 2 Temporal Ctx | 22.7 | 63.3 |
| AD 3 Temporal Ctx | 10.8 | 0.0 |
| AD 4 Temporal Ctx | 14.4 | 24.0 |
| AD 5 Inf Temporal Ctx | 69.7 | 10.0 |
| AD 5 Sup Temporal Ctx | 85.3 | 24.0 |
| AD 6 Inf Temporal Ctx | 67.4 | 50.7 |
| AD 6 Sup Temporal Ctx | 30.8 | 11.4 |
| Control 1 Temporal Ctx | 41.8 | 31.9 |
| Control 2 Temporal Ctx | 23.5 | 25.7 |
| Control 3 Temporal Ctx | 20.9 | 0.0 |
| Control 3 Temporal Ctx | 17.7 | 0.0 |
| Control (Path) 1 Temporal Ctx | 29.1 | 100.0 |
| Control (Path) 2 Temporal Ctx | 21.8 | 19.1 |
| Control (Path) 3 Temporal Ctx | 19.2 | 0.0 |
| Control (Path) 4 Temporal Ctx | 23.8 | 16.4 |
| AD 1 Occipital Ctx | 15.5 | 9.9 |
| AD 2 Occipital Ctx (Missing) | 0.0 | 0.0 |
| AD 3 Occipital Ctx | 13.4 | 0.0 |
| AD 4 Occipital Ctx | 24.3 | 0.0 |
| AD 5 Occipital Ctx | 62.9 | 8.8 |
| AD 6 Occipital Ctx | 50.0 | 26.4 |
| Control 1 Occipital Ctx | 62.4 | 31.6 |
| Control 2 Occipital Ctx | 7.7 | 31.4 |
| Control 3 Occipital Ctx | 33.9 | 0.0 |
| Control 4 Occipital Ctx | 27.7 | 8.7 |
| Control (Path) 1 Occipital Ctx | 100.0 | 18.0 |
| Control (Path) 2 Occipital Ctx | 29.5 | 24.8 |
| Control (Path) 3 Occipital Ctx | 14.6 | 0.0 |
| Control (Path) 4 Occipital Ctx | 42.9 | 87.1 |
| Control 1 Parietal Ctx | 42.0 | 4.1 |
| Control 2 Parietal Ctx | 42.9 | 21.2 |
| Control 3 Parietal Ctx | 8.2 | 0.0 |
| Control (Path) 1 Parietal Ctx | 67.8 | 22.5 |
| Control (Path) 2 Parietal Ctx | 43.8 | 11.2 |
| Control (Path) 3 Parietal Ctx | 11.9 | 4.6 |
| Control (Path) 4 Parietal Ctx | 58.2 | 31.6 |

TABLE XD
General_screening_panel_v1.4

| | Rel. | | Rel. |
|---|---|---|---|
| | Exp. (%) | | Exp. (%) |
| | Ag1800, | | Ag1800, |
| | Run | | Run |
| Tissue Name | 212650191 | Tissue Name | 212650191 |
| Adipose | 1.9 | Renal ca. TK-10 | 1.4 |
| Melanoma* Hs688(A).T | 0.2 | Bladder | 11.5 |
| Melanoma* Hs688(B).T | 0.0 | Gastric ca. (liver met.) NCI-N87 | 2.5 |
| Melanoma* M14 | 0.8 | Gastric ca. KATO III | 0.0 |
| Melanoma* LOXIMVI | 0.0 | Colon ca. SW-948 | 0.6 |
| Melanoma* SK-MEL-5 | 1.4 | Colon ca. SW480 | 2.1 |
| Squamous cell carcinoma SCC-4 | 0.0 | Colon ca.* (SW480 met) SW620 | 0.1 |
| Testis Pool | 0.8 | Colon ca. HT29 | 2.8 |
| Prostate ca.* (bone met) PC-3 | 0.1 | Colon ca. HCT-116 | 5.2 |
| Prostate Pool | 5.0 | Colon ca. CaCo-2 | 2.9 |
| Placenta | 0.4 | Colon cancer tissue | 4.2 |
| Uterus Pool | 2.3 | Colon ca. SW1116 | 0.5 |
| Ovarian ca. OVCAR-3 | 1.3 | Colon ca. Colo-205 | 11.9 |
| Ovarian ca. SK-OV-3 | 1.0 | Colon ca. SW-48 | 5.0 |
| Ovarian ca. OVCAR-4 | 0.2 | Colon Pool | 6.9 |
| Ovarian ca. OVCAR-5 | 10.1 | Small Intestine Pool | 5.7 |
| Ovarian ca. IGROV-1 | 0.9 | Stomach Pool | 3.5 |
| Ovarian ca. OVCAR-8 | 14.9 | Bone Marrow Pool | 4.0 |
| Ovary | 1.4 | Fetal Heart | 2.2 |
| Breast ca. MCF-7 | 64.2 | Heart Pool | 2.6 |
| Breast ca. MDA-MB-231 | 0.2 | Lymph Node Pool | 6.3 |
| Breast ca. BT 549 | 0.0 | Fetal Skeletal Muscle | 0.2 |
| Breast ca. T47D | 31.0 | Skeletal Muscle Pool | 1.1 |
| Breast ca. MDA-N | 0.1 | Spleen Pool | 17.3 |
| Breast Pool | 5.6 | Thymus Pool | 34.6 |
| Trachea | 44.8 | CNS cancer (glio/astro) U87-MG | 1.3 |
| Lung | 4.0 | CNS cancer (glio/astro) U-118-MG | 0.0 |
| Fetal Lung | 3.1 | CNS cancer (neuro; met) SK-N-AS | 0.4 |
| Lung ca. NCI-N417 | 0.7 | CNS cancer (astro) SF-539 | 0.1 |
| Lung ca. LX-1 | 1.8 | CNS cancer (astro) SNB-75 | 0.2 |
| Lung ca. NCI-H146 | 100.0 | CNS cancer (glio) SNB-19 | 1.6 |
| Lung ca. SHP-77 | 23.7 | CNS cancer (glio) SF-295 | 0.1 |
| Lung ca. A549 | 1.9 | Brain (Amygdala) Pool | 0.6 |
| Lung ca. NCI-H526 | 33.4 | Brain (cerebellum) | 33.2 |
| Lung ca. NCI-H23 | 4.4 | Brain (fetal) | 1.3 |
| Lung ca. NCI-H460 | 2.6 | Brain (Hippocampus) Pool | 1.1 |
| Lung ca. HOP-62 | 4.0 | Cerebral Cortex Pool | 0.8 |
| Lung ca. NCI-H522 | 0.2 | Brain (Substantia nigra) Pool | 1.3 |
| Liver | 0.1 | Brain (Thalamus) Pool | 4.9 |
| Fetal Liver | 2.8 | Brain (whole) | 0.9 |
| Liver ca. HepG2 | 0.0 | Spinal Cord Pool | 0.9 |
| Kidney Pool | 8.3 | Adrenal Gland | 2.2 |
| Fetal Kidney | 0.3 | Pituitary gland Pool | 0.4 |
| Renal ca. 786-0 | 0.0 | Salivary Gland | 29.5 |
| Renal ca. A498 | 0.4 | Thyroid (female) | 0.8 |
| Renal ca. ACHN | 0.5 | Pancreatic ca. CAPAN2 | 0.1 |
| Renal ca. UO-31 | 0.1 | Pancreas Pool | 4.4 |

TABLE XE
Oncology_cell_line_screening_panel_v3.1

| | Rel. | | Rel. |
|---|---|---|---|
| | Exp. (%) | | Exp. (%) |
| | Ag1800, | | Ag1800, |
| | Run | | Run |
| Tissue Name | 223128987 | Tissue Name | 223128987 |
| Daoy Medulloblastoma/Cerebellum | 0.3 | Ca Ski_Cervical epidermoid | 0.0 |
| | | carcinoma (metastasis) | |
| TE671 Medulloblastom/Cerebellum | 0.8 | ES-2_Ovarian clear cell carcinoma | 0.0 |
| D283 Med | 1.6 | Ramos/6 h stim_Stimulated with | 9.4 |
| Medulloblastoma/Cerebellum | | PMA/ionomycin 6 h | |
| PFSK-1 Primitive | 0.2 | Ramos/14 h stim_Stimulated with | 11.1 |
| Neuroectodermal/Cerebellum | | PMA/ionomycin 14 h | |
| XF-498_CNS | 1.3 | MEG-01_Chronic myelogenous | 16.8 |
| | | leukemia (megokaryoblast) | |
| SNB-78_CNS/glioma | 0.7 | Raji_Burkitt's lymphoma | 13.5 |
| SF-268_CNS/glioblastoma | 0.0 | Daudi_Burkitt's lymphoma | 38.2 |
| T98G_Glioblastoma | 0.0 | U266_B-cell | 4.3 |
| | | plasmacytoma/myeloma | |
| SK-N-SH_Neuroblastoma | 0.3 | CA46_Burkitt's lymphoma | 16.7 |
| (metastasis) | | | |
| SF-295_CNS/glioblastoma | 0.0 | RL_non-Hodgkin's B-cell lymphoma | 9.2 |
| Cerebellum | 4.6 | JM1_pre-B-cell lymphoma/leukemia | 23.2 |
| Cerebellum | 8.8 | Jurkat_T cell leukemia | 27.2 |
| NCI-H292_Mucoepidermoid lung | 0.3 | TF-1_Erythroleukemia | 24.8 |
| ca. | | | |
| DMS-114_Small cell lung cancer | 1.1 | HUT 78_T-cell lymphoma | 32.5 |
| DMS-79_Small cell lung | 15.7 | U937_Histiocytic lymphoma | 26.2 |
| cancer/neuroendocrine | | | |
| NCI-H146_Small cell lung | 71.2 | KU-812_Myelogenous leukemia | 4.7 |
| cancer/neuroendocrine | | | |
| NCI-H526_Small cell lung | 39.0 | 769-P_Clear cell renal ca. | 0.0 |
| cancer/neuroendocrine | | | |
| NCI-N417_Small cell lung | 0.8 | Caki-2_Clear cell renal ca. | 1.1 |
| cancer/neuroendocrine | | | |
| NCI-H82_Small cell lung | 0.2 | SW 839_Clear cell renal ca. | 0.4 |
| cancer/neuroendocrine | | | |
| NCI-H157_Squamous cell lung | 1.0 | G401_Wilms' tumor | 0.4 |
| cancer (metastasis) | | | |
| NCI-H1155_Large cell lung | 0.2 | Hs766T_Pancreatic ca. (LN | 0.3 |
| cancer/neuroendocrine | | metastasis) | |
| NCI-H1299_Large cell lung | 0.1 | CAPAN-1_Pancreatic | 0.7 |
| cancer/neuroendocrine | | adenocarcinoma (liver metastasis) | |
| NCI-H727_Lung carcinoid | 50.0 | SU86.86_Pancreatic carcinoma | 2.8 |
| | | (liver metastasis) | |
| NCI-UMC-11_Lung carcinoid | 100.0 | BxPC-3_Pancreatic adenocarcinoma | 0.0 |
| LX-1_Small cell lung cancer | 1.2 | HPAC_Pancreatic adenocarcinoma | 0.3 |
| Colo-205_Colon cancer | 9.0 | MIA PaCa-2_Pancreatic ca. | 0.5 |
| KM12_Colon cancer | 0.1 | CFPAC-1_Pancreatic ductal | 2.8 |
| | | adenocarcinoma | |
| KM20L2_Colon cancer | 2.2 | PANC-1_Pancreatic epithelioid | 1.2 |
| | | ductal ca. | |
| NCI-H716_Colon cancer | 5.7 | T24_Bladder ca. (transitional cell) | 0.0 |
| SW-48_Colon adenocarcinoma | 5.8 | 5637_Bladder ca. | 0.0 |
| SW1116_Colon adenocarcinoma | 0.1 | HT-1197_Bladder ca. | 0.0 |
| LS 174T_Colon adenocarcinoma | 1.4 | UM-UC-3_Bladder ca. (transitional | 0.1 |
| | | cell) | |
| SW-948_Colon adenocarcinoma | 0.4 | A204_Rhabdomyosarcoma | 0.2 |
| SW-480_Colon adenocarcinoma | 2.2 | HT-1080_Fibrosarcoma | 0.1 |
| NCI-SNU-5_Gastric ca. | 0.8 | MG-63_Osteosarcoma (bone) | 0.4 |
| KATO III_Stomach | 0.2 | SK-LMS-1_Leiomyosarcoma | 0.0 |
| | | (vulva) | |
| NCI-SNU-16_Gastric ca. | 0.0 | SJRH30_Rhabdomyosarcoma (met | 0.3 |
| | | to bone marrow) | |
| NCI-SNU-1_Gastric ca. | 7.1 | A431_Epidermoid ca. | 6.6 |
| RF-1_Gastric adenocarcinoma | 6.0 | WM266-4_Melanoma | 0.1 |
| RF-48_Gastric adenocarcinoma | 5.3 | DU 145_Prostate | 0.4 |
| MKN-45_Gastric ca. | 2.3 | MDA-MB-468_Breast | 0.3 |
| | | adenocarcinoma | |
| NCI-N87_Gastric ca. | 0.7 | SSC-4_Tongue | 0.1 |
| OVCAR-5_Ovarian ca. | 0.7 | SSC-9_Tongue | 0.0 |
| RL95-2_Uterine carcinoma | 0.0 | SSC-15_Tongue | 0.0 |
| HelaS3_Cervical adenocarcinoma | 0.3 | CAL 27_Squamous cell ca. of | 0.0 |
| | | tongue | |

TABLE XF
Panel 1.3D

| | Rel. | Rel. |
|---|---|---|
| | Exp. (%) | Exp. (%) |
| | Ag1800, | Ag3265, |
| | Run | Run |
| Tissue Name | 156420145 | 165296299 |
| Liver adenocarcinoma | 0.1 | 0.2 |
| Pancreas | 10.4 | 17.4 |
| Pancreatic ca. CAPAN 2 | 0.0 | 0.8 |
| Adrenal gland | 4.4 | 6.9 |
| Thyroid | 5.0 | 4.8 |
| Salivary gland | 24.8 | 57.8 |
| Pituitary gland | 0.6 | 1.6 |
| Brain (fetal) | 0.5 | 2.3 |
| Brain (whole) | 0.7 | 5.6 |
| Brain (amygdala) | 0.9 | 2.3 |
| Brain (cerebellum) | 3.4 | 97.3 |
| Brain (hippocampus) | 4.3 | 5.4 |
| Brain (substantia nigra) | 7.3 | 5.9 |
| Brain (thalamus) | 5.6 | 19.6 |
| Cerebral Cortex | 0.5 | 0.2 |
| Spinal cord | 0.1 | 1.4 |
| glio/astro U87-MG | 0.4 | 0.2 |
| glio/astro U-118-MG | 0.0 | 0.0 |
| astrocytoma SW1783 | 0.0 | 0.0 |
| neuro*; met SK-N-AS | 0.1 | 0.0 |
| astrocytoma SF-539 | 0.1 | 0.0 |
| astrocytoma SNB-75 | 0.4 | 1.5 |
| glioma SNB-19 | 0.2 | 0.2 |
| glioma U251 | 0.1 | 0.2 |
| glioma SF-295 | 0.2 | 0.0 |
| Heart (fetal) | 5.3 | 2.9 |
| Heart | 0.2 | 2.3 |
| Skeletal muscle (fetal) | 3.2 | 0.4 |
| Skeletal muscle | 0.1 | 1.2 |
| Bone marrow | 19.9 | 52.1 |
| Thymus | 100.0 | 100.0 |
| Spleen | 51.4 | 46.7 |
| Lymph node | 17.4 | 86.5 |
| Colorectal | 26.2 | 11.5 |
| Stomach | 21.6 | 27.2 |
| Small intestine | 16.7 | 54.7 |
| Colon ca. SW480 | 1.5 | 0.4 |
| Colon ca.* SW620(SW480 met) | 0.0 | 0.0 |
| Colon ca. HT29 | 0.5 | 0.0 |
| Colon ca. HCT-116 | 0.3 | 1.4 |
| Colon ca. CaCo-2 | 0.4 | 1.9 |
| Colon ca. tissue(ODO3866) | 1.5 | 1.1 |
| Colon ca. HCC-2998 | 2.5 | 2.6 |
| Gastric ca.* (liver met) NCI-N87 | 1.6 | 3.1 |
| Bladder | 0.7 | 1.5 |
| Trachea | 98.6 | 85.3 |
| Kidney | 1.0 | 3.3 |
| Kidney (fetal) | 2.2 | 1.2 |
| Renal ca. 786-0 | 0.0 | 0.0 |
| Renal ca. A498 | 2.1 | 1.7 |
| Renal ca. RXF 393 | 0.1 | 0.0 |
| Renal ca. ACHN | 0.2 | 0.7 |
| Renal ca. UO-31 | 0.0 | 0.0 |
| Renal ca. TK-10 | 0.1 | 0.2 |
| Liver | 0.7 | 1.8 |
| Liver (fetal) | 3.3 | 12.5 |
| Liver ca. (hepatoblast) HepG2 | 0.0 | 0.0 |
| Lung | 7.7 | 13.5 |
| Lung (fetal) | 1.6 | 3.1 |
| Lung ca. (small cell) LX-1 | 0.3 | 0.7 |
| Lung ca. (small cell) NCI-H69 | 9.5 | 9.7 |
| Lung ca. (s. cell var.) SHP-77 | 5.0 | 9.7 |
| Lung ca. (large cell) NCI-H460 | 0.8 | 5.1 |
| Lung ca. (non-sm. cell) A549 | 1.1 | 2.9 |
| Lung ca. (non-s. cell) NCI-H23 | 0.7 | 1.9 |
| Lung ca. (non-s. cell) HOP-62 | 0.5 | 4.8 |
| Lung ca. (non-s. cl) NCI-H522 | 0.2 | 0.0 |
| Lung ca. (squam.) SW 900 | 0.3 | 1.6 |
| Lung ca. (squam.) NCI-H596 | 2.0 | 9.9 |
| Mammary gland | 2.0 | 3.9 |
| Breast ca.* (pl. ef) MCF-7 | 5.9 | 15.5 |
| Breast ca.* (pl. ef) MDA-MB-231 | 0.2 | 0.0 |
| Breast ca.* (pl. ef) T47D | 0.5 | 0.5 |
| Breast ca. BT-549 | 0.0 | 0.0 |
| Breast ca. MDA-N | 0.0 | 0.0 |
| Ovary | 2.4 | 1.3 |
| Ovarian ca. OVCAR-3 | 0.1 | 1.1 |
| Ovarian ca. OVCAR-4 | 0.0 | 0.0 |
| Ovarian ca. OVCAR-5 | 1.6 | 1.0 |
| Ovarian ca. OVCAR-8 | 2.1 | 2.0 |
| Ovarian ca. IGROV-1 | 0.2 | 0.0 |
| Ovarian ca.* (ascites) SK-OV-3 | 0.1 | 0.6 |
| Uterus | 2.4 | 7.6 |
| Placenta | 1.5 | 2.2 |
| Prostate | 6.5 | 24.8 |
| Prostate ca.* (bone met)PC-3 | 0.0 | 0.2 |
| Testis | 1.7 | 3.5 |
| Melanoma Hs688(A).T | 0.0 | 0.0 |
| Melanoma* (met) Hs688(B).T | 0.0 | 0.3 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.1 | 1.3 |
| Adipose | 1.8 | 1.6 |

TABLE XG
Panel 2.2

| | Rel. | | Rel. |
|---|---|---|---|
| | Ex. (%) | | Exp. (%) |
| | Ag3265, | | Ag3265, |
| | Run | | Run |
| Tissue Name | 173762634 | Tissue Name | 173762634 |
| Normal Colon | 49.0 | Kidney Margin (OD04348) | 12.9 |
| Colon cancer (OD06064) | 18.2 | Kidney malignant cancer | 6.4 |
| | | (OD06204B) | |
| Colon Margin (OD06064) | 55.5 | Kidney normal adjacent tissue | 1.8 |
| | | (OD06204E) | |
| Colon cancer (OD06159) | 10.7 | Kidney Cancer (OD04450-01) | 1.3 |
| Colon Margin (OD06159) | 31.0 | Kidney Margin (OD04450-03) | 2.2 |
| Colon cancer (OD06297-04) | 7.9 | Kidney Cancer 8120613 | 1.2 |
| Colon Margin (OD06297-05) | 42.9 | Kidney Margin 8120614 | 4.9 |
| CC Gr.2 ascend colon (ODO3921) | 26.1 | Kidney Cancer 9010320 | 14.2 |
| CC Margin (ODO3921) | 31.0 | Kidney Margin 9010321 | 4.2 |
| Colon cancer metastasis | 10.0 | Kidney Cancer 8120607 | 4.0 |
| (OD06104) | | | |
| Lung Margin (OD06104) | 13.0 | Kidney Margin 8120608 | 8.9 |
| Colon mets to lung (OD04451-01) | 35.1 | Normal Uterus | 6.3 |
| Lung Margin (OD04451-02) | 15.5 | Uterine Cancer 064011 | 3.6 |
| Normal Prostate | 19.1 | Normal Thyroid | 0.0 |
| Prostate Cancer (OD04410) | 4.9 | Thyroid Cancer 064010 | 2.2 |
| Prostate Margin (OD04410) | 2.8 | Thyroid Cancer A302152 | 4.5 |
| Normal Ovary | 10.3 | Thyroid Margin A302153 | 0.0 |
| Ovarian cancer (OD06283-03) | 3.9 | Normal Breast | 5.3 |
| Ovarian Margin (OD06283-07) | 11.3 | Breast Cancer (OD04566) | 1.8 |
| Ovarian Cancer 064008 | 19.5 | Breast Cancer 1024 | 17.0 |
| Ovarian cancer (OD06145) | 9.3 | Breast Cancer (OD04590-01) | 21.6 |
| Ovarian Margin (OD06145) | 9.4 | Breast Cancer Mets | 52.9 |
| | | (OD04590-03) | |
| Ovarian cancer (OD06455-03) | 0.0 | Breast Cancer Metastasis | 84.7 |
| | | (OD04655-05) | |
| Ovarian Margin (OD06455-07) | 2.6 | Breast Cancer 064006 | 13.1 |
| Normal Lung | 18.2 | Breast Cancer 9100266 | 26.6 |
| Invasive poor diff. lung adeno | 19.3 | Breast Margin 9100265 | 5.4 |
| (ODO4945-01 | | | |
| Lung Margin (ODO4945-03) | 18.2 | Breast Cancer A209073 | 4.8 |
| Lung Malignant Cancer | 66.0 | Breast Margin A2090734 | 13.4 |
| (OD03126) | | | |
| Lung Margin (OD03126) | 12.3 | Breast cancer (OD06083) | 32.8 |
| Lung Cancer (OD05014A) | 20.4 | Breast cancer node metastasis | 21.9 |
| | | (OD06083) | |
| Lung Margin (OD05014B) | 8.4 | Normal Liver | 5.7 |
| Lung cancer (OD06081) | 3.2 | Liver Cancer 1026 | 6.9 |
| Lung Margin (OD06081) | 9.0 | Liver Cancer 1025 | 8.7 |
| Lung Cancer (OD04237-01) | 17.1 | Liver Cancer 6004-T | 10.3 |
| Lung Margin (OD04237-02) | 19.9 | Liver Tissue 6004-N | 8.1 |
| Ocular Melanoma Metastasis | 2.3 | Liver Cancer 6005-T | 8.2 |
| Ocular Melanoma Margin (Liver) | 0.9 | Liver Tissue 6005-N | 14.3 |
| Melanoma Metastasis | 1.2 | Liver Cancer 064003 | 7.2 |
| Melanoma Margin (Lung) | 6.5 | Normal Bladder | 17.8 |
| Normal Kidney | 6.8 | Bladder Cancer 1023 | 12.9 |
| Kidney Ca, Nuclear grade 2 | 9.0 | Bladder Cancer A302173 | 12.8 |
| (OD04338) | | | |
| Kidney Margin (OD04338) | 2.8 | Normal Stomach | 100.0 |
| Kidney Ca Nuclear grade 1/2 | 11.3 | Gastric Cancer 9060397 | 36.9 |
| (OD04339) | | | |
| Kidney Margin (OD04339) | 3.1 | Stomach Margin 9060396 | 62.4 |
| Kidney Ca, Clear cell type | 4.5 | Gastric Cancer 9060395 | 59.0 |
| (OD04340) | | | |
| Kidney Margin (OD04340) | 9.1 | Stomach Margin 9060394 | 99.3 |
| Kidney Ca, Nuclear grade 3 | 2.8 | Gastric Cancer 064005 | 43.2 |
| (OD04348) | | | |

TABLE XH
Panel 2D

| | Rel. | | Rel. |
|---|---|---|---|
| | Ep. (%) | | Exp. (%) |
| | Ag1800, | | Ag1800, |
| | Run | | Run |
| Tissue Name | 156420700 | Tissue Name | 156420700 |
| Normal Colon | 26.6 | Kidney Margin 8120608 | 2.9 |
| CC Well to Mod Diff (ODO3866) | 2.8 | Kidney Cancer 8120613 | 1.4 |
| CC Margin (ODO3866) | 20.7 | Kidney Margin 8120614 | 3.6 |
| CC Gr.2 rectosigmoid (ODO3868) | 6.3 | Kidney Cancer 9010320 | 7.6 |
| CC Margin (ODO3868) | 4.2 | Kidney Margin 9010321 | 2.2 |
| CC Mod Diff (ODO3920) | 20.2 | Normal Uterus | 1.8 |
| CC Margin (ODO3920) | 20.4 | Uterus Cancer 064011 | 2.4 |
| CC Gr.2 ascend colon (ODO3921) | 14.8 | Normal Thyroid | 2.5 |
| CC Margin (ODO3921) | 9.0 | Thyroid Cancer 064010 | 2.7 |
| CC from Partial Hepatectomy | 10.6 | Thyroid Cancer A302152 | 3.1 |
| (ODO4309) Mets | | | |
| Liver Margin (ODO4309) | 1.7 | Thyroid Margin A302153 | 4.1 |
| Colon mets to lung (OD04451-01) | 7.5 | Normal Breast | 11.6 |
| Lung Margin (OD04451-02) | 5.8 | Breast Cancer (OD04566) | 7.3 |
| Normal Prostate 6546-1 | 1.5 | Breast Cancer (OD04590-01) | 9.7 |
| Prostate Cancer (OD04410) | 13.7 | Breast Cancer Mets | 85.9 |
| | | (OD04590-03) | |
| Prostate Margin (OD04410) | 10.0 | Breast Cancer Metastasis | 32.5 |
| | | (OD04655-05) | |
| Prostate Cancer (OD04720-01) | 5.7 | Breast Cancer 064006 | 11.3 |
| Prostate Margin (OD04720-02) | 5.2 | Breast Cancer 1024 | 12.1 |
| Normal Lung 061010 | 21.3 | Breast Cancer 9100266 | 25.9 |
| Lung Met to Muscle (ODO4286) | 1.9 | Breast Margin 9100265 | 4.3 |
| Muscle Margin (ODO4286) | 0.5 | Breast Cancer A209073 | 7.5 |
| Lung Malignant Cancer (OD03126) | 100.0 | Breast Margin A209073 | 4.3 |
| Lung Margin (OD03126) | 16.5 | Normal Liver | 1.4 |
| Lung Cancer (OD04404) | 6.4 | Liver Cancer 064003 | 0.6 |
| Lung Margin (OD04404) | 7.1 | Liver Cancer 1025 | 1.7 |
| Lung Cancer (OD04565) | 1.8 | Liver Cancer 1026 | 2.0 |
| Lung Margin (OD04565) | 7.4 | Liver Cancer 6004-T | 1.8 |
| Lung Cancer (OD04237-01) | 17.2 | Liver Tissue 6004-N | 2.0 |
| Lung Margin (OD04237-02) | 9.0 | Liver Cancer 6005-T | 1.9 |
| Ocular Mel Met to Liver | 0.3 | Liver Tissue 6005-N | 0.7 |
| (ODO4310) | | | |
| Liver Margin (ODO4310) | 0.6 | Normal Bladder | 11.6 |
| Melanoma Mets to Lung (OD04321) | 0.5 | Bladder Cancer 1023 | 2.0 |
| Lung Margin (OD04321) | 12.1 | Bladder Cancer A302173 | 2.2 |
| Normal Kidney | 2.0 | Bladder Cancer (OD04718-01) | 2.5 |
| Kidney Ca, Nuclear grade 2 | 6.3 | Bladder Normal Adjacent | 6.6 |
| (OD04338) | | (OD04718-03) | |
| Kidney Margin (OD04338) | 6.4 | Normal Ovary | 1.9 |
| Kidney Ca Nuclear grade 1/2 | 3.7 | Ovarian Cancer 064008 | 9.3 |
| (OD04339) | | | |
| Kidney Margin (OD04339) | 2.5 | Ovarian Cancer (OD04768-07) | 0.8 |
| Kidney Ca, Clear cell type | 8.0 | Ovary Margin (OD04768-08) | 2.9 |
| (OD04340) | | | |
| Kidney Margin (OD04340) | 3.0 | Normal Stomach | 17.0 |
| Kidney Ca, Nuclear grade 3 | 1.5 | Gastric Cancer 9060358 | 2.8 |
| (OD04348) | | | |
| Kidney Margin (OD04348) | 1.9 | Stomach Margin 9060359 | 19.2 |
| Kidney Cancer (OD04622-01) | 6.5 | Gastric Cancer 9060395 | 8.4 |
| Kidney Margin (OD04622-03) | 2.8 | Stomach Margin 9060394 | 28.7 |
| Kidney Cancer (OD04450-01) | 0.2 | Gastric Cancer 9060397 | 56.6 |
| Kidney Margin (OD04450-03) | 3.1 | Stomach Margin 9060396 | 44.8 |
| Kidney Cancer 8120607 | 0.7 | Gastric Cancer 064005 | 16.0 |

TABLE XI
Panel 3D

| | Rel. | | Rel. |
|---|---|---|---|
| | Ex. (%) | | Exp. (%) |
| | Ag3265, | | Ag3265, |
| | Run | | Run |
| Tissue Name | 165468234 | Tissue Name | 165468234 |
| Daoy-Medulloblastoma | 0.5 | Ca Ski-Cervical epidermoid | 0.5 |
| | | carcinoma (metastasis) | |
| TE671-Medulloblastoma | 0.3 | ES-2-Ovarian clear cell carcinoma | 0.0 |
| D283 Med-Medulloblastoma | 0.3 | Ramos-Stimulated with | 7.0 |
| | | PMA/ionomycin 6 h | |
| PFSK-1-Primitive | 0.0 | Ramos-Stimulated with | 15.9 |
| Neuroectodermal | | PMA/ionomycin 14 h | |
| XF-498-CNS | 0.9 | MEG-01-Chronic myelogenous | 30.4 |
| | | leukemia (megokaryoblast) | |
| SNB-78-Glioma | 0.2 | Raji-Burkitt's lymphoma | 11.8 |
| SF-268-Glioblastoma | 0.0 | Daudi-Burkitt's lymphoma | 45.4 |
| T98G-Glioblastoma | 0.0 | U266-B-cell plasmacytoma | 10.5 |
| SK-N-SH-Neuroblastoma | 0.9 | CA46-Burkitt's lymphoma | 9.7 |
| (metastasis) | | | |
| SF-295-Glioblastoma | 0.0 | RL-non-Hodgkin's B-cell | 2.4 |
| | | lymphoma | |
| Cerebellum | 7.2 | JM1-pre-B-cell lymphoma | 17.3 |
| Cerebellum | 16.0 | Jurkat-T cell leukemia | 47.0 |
| NCI-H292-Mucoepidermoid | 0.5 | TF-1-Erythroleukemia | 18.8 |
| lung carcinoma | | | |
| DMS-114-Small cell lung | 1.8 | HUT 78-T-cell lymphoma | 12.8 |
| cancer | | | |
| DMS-79-Small cell lung cancer | 97.3 | U937-Histiocytic lymphoma | 17.2 |
| NCI-H146-Small cell lung | 97.9 | KU-812-Myelogenous leukemia | 7.8 |
| cancer | | | |
| NCI-H526-Small cell lung | 50.3 | 769-P-Clear cell renal carcinoma | 0.0 |
| cancer | | | |
| NCI-N417-Small cell lung | 0.5 | Caki-2-Clear cell renal carcinoma | 1.2 |
| cancer | | | |
| NCI-H82-Small cell lung cancer | 0.4 | SW 839-Clear cell renal carcinoma | 0.0 |
| NCI-H157-Squamous cell lung | 0.0 | Rhabdoid kidney tumor | 0.1 |
| cancer (metastasis) | | | |
| NCI-H1155-Large cell lung | 0.4 | Hs766T-Pancreatic carcinoma (LN | 0.7 |
| cancer | | metastasis) | |
| NCI-H1299-Large cell lung | 0.2 | CAPAN-1-Pancreatic | 0.6 |
| cancer | | adenocarcinoma (liver metastasis) | |
| NCI-H727-Lung carcinoid | 100.0 | SU86.86-Pancreatic carcinoma | 0.9 |
| | | (liver metastasis) | |
| NCI-UMC-11-Lung carcinoid | 78.5 | BxPC-3-Pancreatic | 0.0 |
| | | adenocarcinoma | |
| LX-1-Small cell lung cancer | 2.7 | HPAC-Pancreatic adenocarcinoma | 0.2 |
| Colo-205-Colon cancer | 12.6 | MIA PaCa-2-Pancreatic carcinoma | 0.4 |
| KM12-Colon cancer | 0.1 | CFPAC-1-Pancreatic ductal | 0.6 |
| | | adenocarcinoma | |
| KM20L2-Colon cancer | 2.6 | PANC-1-Pancreatic epithelioid | 2.6 |
| | | ductal carcinoma | |
| NCI-H716-Colon cancer | 1.5 | T24-Bladder carcinma (transitional | 0.1 |
| | | cell) | |
| SW-48-Colon adenocarcinoma | 7.3 | 5637-Bladder carcinoma | 0.1 |
| SW1116-Colon adenocarcinoma | 1.1 | HT-1197-Bladder carcinoma | 0.0 |
| LS 174T-Colon adenocarcinoma | 1.8 | UM-UC-3-Bladder carcinma | 0.0 |
| | | (transitional cell) | |
| SW-948-Colon adenocarcinoma | 0.0 | A204-Rhabdomyosarcoma | 0.1 |
| SW-480-Colon adenocarcnioma | 2.8 | HT-1080-Fibrosarcoma | 0.2 |
| NCI-SNU-5-Gastric carcinoma | 1.0 | MG-63-Osteosarcoma | 0.3 |
| KATO III-Gastric carcinoma | 0.0 | SK-LMS-1-Leiomyosarcoma | 0.1 |
| | | (vulva) | |
| NCI-SNU-16-Gastric carcinoma | 0.0 | SJRH30-Rhabdomyosarcoma (met | 0.1 |
| | | to bone marrow) | |
| NCI-SNU-1-Gastric carcinoma | 10.3 | A431-Epidermoid carcinoma | 0.4 |
| RF-1-Gastric adenocarcinoma | 10.3 | WM266-4-Melanoma | 0.0 |
| RF-48-Gastric adenocarcinoma | 5.6 | DU 145-Prostate carcinoma (brain | 0.0 |
| | | metastasis) | |
| MKN-45-Gastric carcinoma | 2.6 | MDA-MB-468-Breast | 0.2 |
| | | adenocarcinoma | |
| NCI-N87-Gastric carcinoma | 0.2 | SCC-4-Squamous cell carcinoma of | 0.0 |
| | | tongue | |
| OVCAR-5-Ovarian carcinoma | 0.6 | SCC-9-Squamous cell carcinoma of | 0.0 |
| | | tongue | |
| RL95-2-Uterine carcinoma | 0.0 | SCC-15-Squamous cell carcinoma | 0.0 |
| | | of tongue | |
| HelaS3-Cervical | 0.0 | CAL 27-Squamous cell carcinoma | 0.0 |
| adenocarcinoma | | of tongue | |

TABLE XJ
Panel 4.1D

| | Rel. | | Rel. |
|---|---|---|---|
| | Exp. () | | Exp. (%) |
| | Ag3265, | | Ag3265, |
| | Run | | Run |
| Tissue Name | 169827303 | Tissue Name | 169827303 |
| Secondary Th1 act | 50.3 | HUVEC IL-1beta | 0.4 |
| Secondary Th2 act | 83.5 | HUVEC IFN gamma | 0.0 |
| Secondary Tr1 act | 96.6 | HUVEC TNF alpha + IFN gamma | 0.0 |
| Secondary Th1 rest | 47.0 | HUVEC TNF alpha + IL4 | 0.0 |
| Secondary Th2 rest | 100.0 | HUVEC IL-11 | 0.7 |
| Secondary Tr1 rest | 76.8 | Lung Microvascular EC none | 0.4 |
| Primary Th1 act | 36.1 | Lung Microvascular EC TNF alpha + IL-1beta | 0.0 |
| Primary Th2 act | 66.4 | Microvascular Dermal EC none | 1.6 |
| Primary Tr1 act | 55.9 | Microsvasular Dermal EC | 1.0 |
| | | TNF alpha + IL-1beta | |
| Primary Th1 rest | 56.6 | Bronchial epithelium TNF alpha + IL1beta | 0.0 |
| Primary Th2 rest | 56.6 | Small airway epithelium none | 0.7 |
| Primary Tr1 rest | 79.6 | Small airway epithelium TNF alpha + IL-1beta | 0.4 |
| CD45RA CD4 lymphocyte act | 24.7 | Coronery artery SMC rest | 0.0 |
| CD45RO CD4 lymphocyte act | 55.9 | Coronery artery SMC TNF alpha + IL-1beta | 0.7 |
| CD8 lymphocyte act | 88.3 | Astrocytes rest | 0.0 |
| Secondary CD8 lymphocyte rest | 54.3 | Astrocytes TNF alpha + IL-1beta | 0.0 |
| Secondary CD8 lymphocyte act | 48.3 | KU-812 (Basophil) rest | 16.2 |
| CD4 lymphocyte none | 42.9 | KU-812 (Basophil) | 23.0 |
| | | PMA/ionomycin | |
| 2ry Th1/Th2/Tr1_anti-CD95 | 71.2 | CCD1106 (Keratinocytes) none | 0.5 |
| CH11 | | | |
| LAK cells rest | 49.3 | CCD1106 (Keratinocytes) | 1.3 |
| | | TNF alpha + IL-1beta | |
| LAK cells IL-2 | 68.3 | Liver cirrhosis | 6.2 |
| LAK cells IL-2 + IL-12 | 34.9 | NCI-H292 none | 0.4 |
| LAK cells IL-2 + IFN gamma | 62.0 | NCI-H292 IL-4 | 0.0 |
| LAK cells IL-2 + IL-18 | 40.9 | NCI-H292 IL-9 | 0.6 |
| LAK cells PMA/ionomycin | 17.9 | NCI-H292 IL-13 | 0.5 |
| NK Cells IL-2 rest | 98.6 | NCI-H292 IFN gamma | 0.0 |
| Two Way MLR 3 day | 46.0 | HPAEC none | 0.0 |
| Two Way MLR 5 day | 55.1 | HPAEC TNF alpha + IL-1beta | 0.4 |
| Two Way MLR 7 day | 55.5 | Lung fibroblast none | 2.0 |
| PBMC rest | 45.7 | Lung fibroblast TNF alpha + IL-1beta | 4.3 |
| PBMC PWM | 49.3 | Lung fibroblast IL-4 | 3.2 |
| PBMC PHA-L | 63.3 | Lung fibroblast IL-9 | 4.1 |
| Ramos (B cell) none | 48.3 | Lung fibroblast IL-13 | 2.2 |
| Ramos (B cell) ionomycin | 42.9 | Lung fibroblast IFN gamma | 1.4 |
| B lymphocytes PWM | 29.5 | Dermal fibroblast CCD1070 rest | 0.0 |
| B lymphocytes CD40L and IL-4 | 85.9 | Dermal fibroblast CCD1070 TNF | 69.3 |
| | | alpha | 0.0 |
| EOL-1 dbcAMP | 67.4 | Dermal fibroblast CCD1070 IL-1beta | 0.3 |
| EOL-1 dbcAMP | 51.8 | Dermal fibroblast IFN gamma | 12.0 |
| PMA/ionomycin | | | |
| Dendritic cells none | 54.3 | Dermal fibroblast IL-4 | 20.2 |
| Dendritic cells LPS | 17.3 | Dermal Fibroblast rest | 10.4 |
| Dendritic cells anti-CD40 | 55.9 | Neutrophils TNFa + LPS | 12.9 |
| Monocytes rest | 19.8 | Neutrophils rest | 73.2 |
| Monocytes LPS | 25.9 | Colon | 21.6 |
| Macrophages rest | 43.5 | Lung | 6.1 |
| Macrophages LPS | 13.8 | Thymus | 88.3 |
| HUVEC none | 0.0 | Kidney | 4.2 |
| HUVEC starved | 2.0 | | |

TABLE XK
Panel 4D

| | Rel. | | Rel. |
|---|---|---|---|
| | Exp (%) | | Exp. (%) |
| | Ag1800, | | Ag1800, |
| | Run | | Run |
| Tissue Name | 156420767 | Tissue Name | 156420767 |
| Secondary Th1 act | 23.0 | HUVEC IL-1beta | 0.0 |
| Secondary Th2 act | 38.2 | HUVEC IFN gamma | 0.1 |
| Secondary Tr1 act | 59.0 | HUVEC TNF alpha + IFN gamma | 0.0 |
| Secondary Th1 rest | 14.0 | HUVEC TNF alpha + IL4 | 0.0 |
| Secondary Th2 rest | 30.1 | HUVEC IL-11 | 0.1 |
| Secondary Tr1 rest | 23.5 | Lung Microvascular EC none | 0.0 |
| Primary Th1 act | 8.8 | Lung Microvascular EC TNF alpha + IL-1beta | 0.1 |
| Primary Th2 act | 15.6 | Microvascular Dermal EC none | 0.5 |
| Primary Tr1 act | 18.0 | Microsvasular Dermal EC | 0.1 |
| | | TNF alpha + IL-1beta | |
| Primary Th1 rest | 57.8 | Bronchial epithelium TNF alpha + IL1beta | 0.0 |
| Primary Th2 rest | 29.1 | Small airway epithelium none | 0.0 |
| Primary Tr1 rest | 34.4 | Small airway epithelium TNF alpha + IL-1beta | 0.0 |
| CD45RA CD4 lymphocyte act | 15.2 | Coronery artery SMC rest | 0.4 |
| CD45RO CD4 lymphocyte act | 24.1 | Coronery artery SMC TNF alpha + IL-1beta | 0.0 |
| CD8 lymphocyte act | 13.5 | Astrocytes rest | 0.0 |
| Secondary CD8 lymphocyte rest | 18.9 | Astrocytes TNF alpha + IL-1beta | 0.0 |
| Secondary CD8 lymphocyte act | 12.1 | KU-812 (Basophil) rest | 9.8 |
| CD4 lymphocyte none | 16.6 | KU-812 (Basophil) | 11.7 |
| | | PMA/ionomycin | |
| 2ry Th1/Th2/Tr1_anti-CD95 | 35.6 | CCD1106 (Keratinocytes) none | 0.2 |
| CH11 | | | |
| LAK cells rest | 12.9 | CCD1106 (Keratinocytes) | 0.0 |
| | | TNF alpha + IL-1beta | |
| LAK cells IL-2 | 15.7 | Liver cirrhosis | 3.0 |
| LAK cells IL-2 + IL-12 | 13.5 | Lupus kidney | 0.1 |
| LAK cells IL-2 + IFN gamma | 25.3 | NCI-H292 none | 0.1 |
| LAK cells IL-2 + IL-18 | 23.7 | NCI-H292 IL-4 | 0.3 |
| LAK cells PMA/ionomycin | 8.9 | NCI-H292 IL-9 | 0.3 |
| NK Cells IL-2 rest | 37.4 | NCI-H292 IL-13 | 0.2 |
| Two Way MLR 3 day | 19.8 | NCI-H292 IFN gamma | 0.3 |
| Two Way MLR 5 day | 15.1 | HPAEC none | 0.3 |
| Two Way MLR 7 day | 17.8 | HPAEC TNF alpha + IL-1beta | 0.0 |
| PBMC rest | 12.8 | Lung fibroblast none | 1.8 |
| PBMC PWM | 17.0 | Lung fibroblast TNF alpha + IL-1beta | 0.5 |
| PBMC PHA-L | 19.6 | Lung fibroblast IL-4 | 0.9 |
| Ramos (B cell) none | 13.4 | Lung fibroblast IL-9 | 1.7 |
| Ramos (B cell) ionomycin | 12.9 | Lung fibroblast IL-13 | 1.2 |
| B lymphocytes PWM | 55.5 | Lung fibroblast IFN gamma | 0.3 |
| B lymphocytes CD40L and IL-4 | 100.0 | Dermal fibroblast CCD1070 rest | 1.0 |
| EOL-1 dbcAMP | 40.6 | Dermal fibroblast CCD1070 TNF | 55.1 |
| | | alpha | |
| EOL-1 dbcAMP | 25.5 | Dermal fibroblast CCD1070 IL-1beta | 0.1 |
| PMA/ionomycin | | | |
| Dendritic cells none | 18.2 | Dermal fibroblast IFN gamma | 3.3 |
| Dendritic cells LPS | 3.2 | Dermal fibroblast IL-4 | 6.4 |
| Dendritic cells anti-CD40 | 14.2 | IBD Colitis 2 | 4.2 |
| Monocytes rest | 3.4 | IBD Crohn's | 0.9 |
| Monocytes LPS | 5.5 | Colon | 11.8 |
| Macrophages rest | 14.5 | Lung | 3.6 |
| Macrophages LPS | 5.1 | Thymus | 1.1 |
| HUVEC none | 0.0 | Kidney | 64.2 |
| HUVEC starved | 0.1 | | |

TABLE XL
Panel 5 Islet

| | Rel. | Rel. |
|---|---|---|
| | Exp. (%) | Exp. (%) |
| | Ag1800 | Ag3265, |
| | Run | Run |
| Tissue Name | 279370792 | 172203502 |
| 97457_Patient-02go_adipose | 11.3 | 4.2 |
| 97476_Patient-07sk_skeletal | 0.0 | 6.5 |
| muscle | | |
| 97477_Patient-07ut_uterus | 4.1 | 5.2 |
| 97478_Patient-07pl_placenta | 2.1 | 1.5 |
| 99167_Bayer Patient 1 | 100.0 | 100.0 |
| 97482_Patient-08ut_uterus | 0.7 | 0.0 |
| 97483_Patient-08pl_placenta | 3.9 | 0.0 |
| 97486_Patient-09sk_skeletal | 3.3 | 0.0 |
| muscle | | |
| 97487_Patient-09ut_uterus | 2.8 | 2.0 |
| 97488_Patient-09pl_placenta | 0.0 | 0.0 |
| 97492_Patient-10ut_uterus | 0.0 | 3.6 |
| 97493_Patient-10pl_placenta | 0.9 | 1.1 |
| 97495_Patient-11go_adipose | 0.7 | 3.1 |
| 97496_Patient-11sk_skeletal | 2.8 | 1.1 |
| muscle | | |
| 97497_Patient-11ut_uterus | 3.9 | 3.1 |
| 97498_Patient-11pl_placenta | 1.4 | 0.0 |
| 97500_Patient-12go_adipose | 5.0 | 2.4 |
| 97501_Patient-12sk_skeletal | 9.0 | 0.7 |
| muscle | | |
| 97502_Patient-12ut_uterus | 5.8 | 5.6 |
| 97503_Patient-12pl_placenta | 0.8 | 1.6 |
| 94721_Donor 2 U - A_Mesenchymal | 0.0 | 0.6 |
| Stem Cells | | |
| 94722_Donor 2 U - B_Mesenchymal | 1.0 | 0.4 |
| Stem Cells | | |
| 94723_Donor 2 U - C_Mesenchymal | 0.0 | 0.0 |
| Stem Cells | | |
| 94709_Donor 2 AM - A_adipose | 0.0 | 1.6 |
| 94710_Donor 2 AM - B_adipose | 0.0 | 0.0 |
| 94711_Donor 2 AM - C_adipose | 0.0 | 0.0 |
| 94712_Donor 2 AD - A_adipose | 0.0 | 0.0 |
| 94713_Donor 2 AD - B_adipose | 0.0 | 0.0 |
| 94714_Donor 2 AD - C_adipose | 0.0 | 0.0 |
| 94742_Donor 3 U - A_Mesenchymal | 0.9 | 0.0 |
| Stem Cells | | |
| 94743_Donor 3 U - B_Mesenchymal | 0.8 | 0.0 |
| Stem Cells | | |
| 94730_Donor 3 AM - A_adipose | 0.0 | 0.0 |
| 94731_Donor 3 AM - B_adipose | 0.0 | 0.0 |
| 94732_Donor 3 AM - C_adipose | 0.0 | 0.0 |
| 94733_Donor 3 AD - A_adipose | 1.0 | 0.0 |
| 94734_Donor 3 AD - B_adipose | 0.9 | 0.0 |
| 94735_Donor 3 AD - C_adipose | 0.0 | 0.7 |
| 77138_Liver_HepG2untreated | 0.0 | 0.0 |
| 73556_Heart_Cardiac stromal | 0.0 | 0.0 |
| cells (primary) | | |
| 81735_Small Intestine | 61.6 | 14.0 |
| 72409_Kidney_Proximal Convoluted | 0.0 | 0.0 |
| Tubule | | |
| 82685_Small intestine_Duodenum | 7.4 | 6.7 |
| 90650_Adrenal_Adrenocortical | 1.1 | 1.0 |
| adenoma | | |
| 72410_Kidney_HRCE | 0.0 | 0.0 |
| 72411_Kidney_HRE | 0.0 | 0.0 |
| 73139_Uterus_Uterine smooth | 0.0 | 0.6 |
| muscle cells | | |

TABLE XM
Panel 5D

| | Rel. | | Rel. |
|---|---|---|---|
| | Exp. (%) | | Exp. (%) |
| | Ag365, | | Ag3265, |
| | Run | | Run |
| Tissue Name | 166510703 | Tissue Name | 166510703 |
| 97457_Patient-02go_adipose | 20.3 | 94709_Donor 2 AM - A_adipose | 0.0 |
| 97476_Patient-07sk_skeletal | 15.2 | 94710_Donor 2 AM - B_adipose | 0.0 |
| muscle | | | |
| 97477_Patient-07ut_uterus | 14.8 | 94711_Donor 2 AM - C_adipose | 0.0 |
| 97478_Patient-07pl_placenta | 0.0 | 94712_Donor 2 AD - A_adipose | 0.0 |
| 97481_Patient-08sk_skeletal | 7.3 | 94713_Donor 2 AD - B_adipose | 0.0 |
| muscle | | | |
| 97482_Patient-08ut_uterus | 15.2 | 94714_Donor 2 AD - C_adipose | 0.0 |
| 97483_Patient-08pl_placenta | 5.0 | 94742_Donor 3 U - A_Mesenchymal | 0.0 |
| | | Stem Cells | |
| 97486_Patient-09sk_skeletal | 8.3 | 94743_Donor 3 U - B_Mesenchymal | 0.0 |
| muscle | | Stem Cells | |
| 97487_Patient-09ut_uterus | 0.0 | 94730_Donor 3 AM - A_adipose | 0.0 |
| 97488_Patient-09pl_placenta | 7.5 | 94731_Donor 3 AM - B_adipose | 0.0 |
| 97492_Patient-10ut_uterus | 0.0 | 94732_Donor 3 AM - C_adipose | 0.0 |
| 97493_Patient-10pl_placenta | 5.0 | 94733_Donor 3 AD - A_adipose | 0.0 |
| 97495_Patient-11go_adipose | 6.8 | 94734_Donor 3 AD - B_adipose | 0.0 |
| 97496_Patient-11sk_skeletal | 6.7 | 94735_Donor 3 AD - C_adipose | 5.2 |
| muscle | | | |
| 97497_Patient-11ut_uterus | 0.0 | 77138_Liver_HepG2untreated | 0.0 |
| 97498_Patient-11pl_placenta | 14.6 | 73556_Heart_Cardiac stromal cells | 0.0 |
| | | (primary) | |
| 97500_Patient-12go_adipose | 15.5 | 81735_Small Intestine | 100.0 |
| 97501_Patient-12sk_skeletal | 14.0 | 72409_Kidney_Proximal Convoluted | 0.0 |
| muscle | | Tubule | |
| 97502_Patient-12ut_uterus | 12.7 | 82685_Small intestine_Duodenum | 63.3 |
| 97503_Patient-12pl_placenta | 5.6 | 90650_Adrenal_Adrenocortical | 22.7 |
| | | adenoma | |
| 94721_Donor 2 U - | 0.0 | 72410_Kidney_HRCE | 0.0 |
| A_Mesenchymal Stem Cells | | | |
| 94722_Donor 2 U - | 0.0 | 72411_Kidney_HRE | 0.0 |
| B_Mesenchymal Stem Cells | | | |
| 94723_Donor 2 U - | 0.0 | 73139_Uterus_Uterine smooth | 18.3 |
| C_Mesenchymal Stem Cells | | muscle cells | |

CNS_neurodegeneration_v1.0 Summary: Ag1800 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of this gene in treatment of central nervous system disorders.

General_screening_panel_v1.4 Summary: Ag1800 Highest expression of this gene is detected in lung cancer NCI-H146 cell line (CT=27.5). Moderate to low expression of this gene is also seen in number of cancer cell lines derived from melanoma, brain, colon, renal, lung, breast and ovarian cancers. Therefore, expression of this gene may be used as diagnostic marker to detect the presence of these cancers and also, therapeutic modulation of this gene may be useful in the treatment of these cancers.

Among tissues with metabolic or endocrine function, this gene is expressed at high to moderate to low levels in pancreas, adipose, adrenal gland, thyroid, skeletal muscle, heart, fetal liver and the gastrointestinal tract. Therefore, therapeutic modulation of the activity of this gene may prove useful in the treatment of endocrine/metabolically related diseases, such as obesity and diabetes.

In addition, this gene is expressed at moderate to low levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CT=32.7) when compared to adult liver (CT=37.8). This observation suggests that expression of this gene can be used to distinguish fetal from adult liver. In addition, the relative overexpression of this gene in fetal tissue suggests that the protein product may enhance liver growth or development in the fetus and thus may also act in a regenerative capacity in the adult. Therefore, therapeutic modulation of the protein encoded by this gene could be useful in treatment of liver related diseases.

Oncology_cell_line_screening_panel_v3.1 Summary: Ag1800 Highest expression of this gene is detected in lung cancer NCI-UMC-11 cell line (CT=27.2). Moderate to low expression of this gene is seen in number of cancer cell lines derived from epidermoid carcinoma, T and B cells lymphoma/leukemia, pancreatic, lung, brain and colon cancers. Therefore, therapeutic modulation of this gene may be useful in the treatment of these cancers.

Panel 1.3D Summary: Ag1800/Ag3265 Two experiment with different probe-primer sets are in good agreement. Highest expression of this gene is detected in thymus (CT=27-29). Moderate to low expression of this gene is also seen in all the regions of brain, tissues with metabolic/endocrine functions and number of cancer cell lines derived from ovarian, breast, renal, and lung cancers, which is consistent with the expression profile seen in panel 1.4. Please panel 1.4 for further discussion on the utility of this gene.

Panel 2.2 Summary: Ag3265 Highest expression of this gene is detected in normal stomach (CT=30.7). Moderate to low expression of this gene is seen in normal and cancer samples derived from stomach, bladder, liver, breast, kidney, lung, ovary, and colon. Therefore, therapeutic modulation of this gene may be useful in the treatment of stomach, bladder, liver, breast, kidney, lung, ovary, and colon cancers.

Panel 2D Summary: Ag1800 Highest expression of this gene is detected in malignant lung cancer (CT=26.3). Interestingly, expression of this gene is higher in lung cancer compared to the adjacent normal sample. Therefore, expression of this gene may be used as a diagnostic marker to detect the presence of malignant lung cancer. Similar to expression seen in panel 2.2, expression of this gene is seen in both normal and cancer samples derived from stomach, bladder, liver, breast, kidney, lung, ovary, prostate and colon. Therefore, therapeutic modulation of this gene may be useful in the treatment of stomach, bladder, liver, breast, kidney, lung, ovary, prostate and colon cancers.

Panel 3D Summary: Ag3265 Highest expression of this gene is detected in lung carcinoid (CT=27.4). Moderate to low expression of this gene is seen in number of cancer cell lines derived from T and B cells lymphoma/leukemia, pancreatic, lung, gastric and colon cancers. Therefore, therapeutic modulation of this gene may be useful in the treatment of these cancers.

Panel 4.1D Summary: Ag3265 Highest expression of this gene is detected in resting secondary Th2 cells (CT=30.2). This gene is expressed at moderate to low levels in T lymphocytes prepared under a number of conditions, treated and untreated dendritic cells, monocytes, macrophages, LAK cells, B cells, basophils, activated dermal fibroblasts and normal tissues represented by colon, lung, thymus and kidney. Dendritic cells and macrophages are powerful antigen-presenting cells (APC) whose function is pivotal in the initiation and maintenance of normal immune responses. Autoimmunity and inflammation may also be reduced by suppression of this function. Therefore, small molecule drugs that antagonzie the function of this gene product may reduce or eliminate the symptoms in patients with several types of autoimmune and inflammatory diseases, such as lupus erythematosus, Crohn's disease, ulcerative colitis, multiple sclerosis, chronic obstructive pulmonary disease, asthma, emphysema, rheumatoid arthritis, or psoriasis.

Panel 4D Summary: Ag1800 Highest expression of this gene is detected in CD40L and IL-4 treated B lymphocytes (CT=27.6). Moderate to low expression of this gene is detected in T lymphocytes prepared under a number of conditions, treated and untreated dendritic cells, monocytes, macrophages, LAK cells, B cells, basophils, activated dermal fibroblasts and normal tissues represented by colon, lung, thymus and kidney. Expression profile of this gene in this panel is similar to that in panel 4.1D. Please see panel 4.1D for further discussion of this gene.

Panel 5 Islet Summary: Ag1800/Ag3265 Two experiments with different probe primer sets are in good agreement. Highest expression of this gene is seen in pancreatic islet cells (CTs=30.9-32). Low expression of this gene is also seen in small intestine. This gene codes for SERCA3. SERCA3 is a magnesium dependent enzyme that catalyzes the hydrolysis of ATP coupled with the transport of the calcium. This enzyme transports calcium ions from the cytosol into the sarcoplasmic/endoplasmic reticulum and has a central role in intracellular calcium signaling. Using Curagen GeneCalling studies SERCA3 was found to be up-regulated 7-fold in good insulin-secreting insulinoma cell lines vs poor insulin-secreting insulinoma cell lines. It is known that insulin secretagogues that stimulate intracellular calcium influx also elevate calcium levels in the ER. Thus, SERCA3-mediated calcium uptake into the ER may optimize both beta cell calcium homeostasis and the insulin secretory process. Moreover, literature data have shown that SERCA3 is down-regulated in islet tissue of the diabetic GK rat, further supporting an important role for SERCA3 in insulin secretion The combined data suggest that activation of SERCA3 through the use of small molecule drug may promote beta cell insulin secretion and be an effective treatment for the beta cell secretory defect in Type 2 diabetes.

Varadi A, Lebel L, Hashim Y, Mehta Z, Ashcroft S J, Turner R. Sequence variants of the sarco(endo)plasmic reticulum Ca(2+)-transport ATPase 3 gene SERCA3) in Caucasian type II diabetic patients (UK Prospective Diabetes Study 48).Diabetologia. October 1999;42(10):1240-3. PMID: 10525666; Poch E, Leach S, Snape S, Cacic T, MacLennan D H, Lytton J. Functional characterization of alternatively spliced human SERCA3 transcripts.Am J Physiol. December 1998;275(6 Pt 1):C1449-58. PMID: 9843705; Maechler P, Kennedy E D, Sebo E, Valeva A, Pozzan T, Wollheim C B. Secretagogues modulate the calcium concentration in the endoplasmic reticulum of insulin-secreting cells. Studies in aequorin-expressing intact and permeabilized ins-1 cells.J Biol Chem. Apr. 30, 1999;274(18):12583-92. PMID: 10212237; Varadi A, Molnar E, Ostenson C G, Ashcroft S J. Isoforms of endoplasmic reticulum Ca(2+)-ATPase are differentially expressed in normal and diabetic islets of Langerhans. Biochem J. Oct. 15, 1996;319 (Pt 2):521-7. PMID: 8912690.

Panel 5D Summary: Ag3265 Low expression of this gene is restricted to small intestine. Please see panel 5I and panel 1.4 for further discussion of this gene.

Y. CG56246-01 and CG56246-02: Human Carboxypeptidase A2-Like Protein.

Expression of gene CG56246-01 and CG56246-02 was assessed using the primer-probe set Ag1757, described in Table YA. Results of the RTQ-PCR runs are shown in Tables YB, YC, YD and YE.

TABLE YA

Probe Name Ag1757

| | | | | |
|---|---|---|---|---|
| | | | | SEQ ID |
| Primers | Sequence | Length | Start Position | No |

| | | | | |
|---|---|---|---|---|
| Forward | 5′-aggagaagagaacggagtggta-3′ | 22 | 960 | 458 |
| Probe | TET-5′-ttcaattttggggcctaccataccct-3′-TAMRA | 26 | 932 | 459 |
| Reverse | 5′-aggttatccatttcttgggaaa-3′ | 22 | 902 | 460 |

TABLE YB
Panel 1.3D

| | Rel. | | Rel. |
|---|---|---|---|
| | Exp. %) | | Exp. (%) |
| | Ag1757, | | Ag1757, |
| | Run | | Run |
| Tissue Name | 156016239 | Tissue Name | 156016239 |
| Liver adenocarcinoma | 0.0 | Kidney (fetal) | 0.0 |
| Pancreas | 100.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.0 | Lung | 0.1 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 0.1 | Lung ca. (large cell)NCI-H460 | 0.0 |
| glio/astro U87-MG | 0.0 | Lung ca. (non-sm.cell) A549 | 0.0 |
| glio/astro U-118-MG | 0.0 | Lung ca. (non-s.cell) NCI-H23 | 0.0 |
| astrocytoma SW1783 | 0.0 | Lung ca. (non-s.cell) HOP-62 | 0.0 |
| neuro*; met SK-N-AS | 0.0 | Lung ca. (non-s.cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) MDA-MB-231 | 0.0 |
| Heart (fetal) | 0.0 | Breast ca.* (pl.ef) T47D | 0.0 |
| Heart | 0.0 | Breast ca. BT-549 | 0.0 |
| Skeletal muscle (fetal) | 0.1 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 2.5 | Ovarian ca.* (ascites) SK-OV-3 | 0.0 |
| Small intestine | 0.1 | Uterus | 0.0 |
| Colon ca. SW480 | 0.0 | Placenta | 0.0 |
| Colon ca.* SW620(SW480 met) | 0.0 | Prostate | 0.0 |
| Colon ca. HT29 | 0.0 | Prostate ca.* (bone met)PC-3 | 0.0 |
| Colon ca. HCT-116 | 0.0 | Testis | 0.0 |
| Colon ca. CaCo-2 | 0.2 | Melanoma Hs688(A).T | 0.0 |
| Colon ca. tissue(ODO3866) | 0.0 | Melanoma* (met) Hs688(B).T | 0.0 |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 14.9 | Melanoma LOX IMVI | 0.0 |
| Trachea | 0.0 | Melanoma* (met) SK-MEL-5 | 0.0 |
| Kidney | 0.0 | Adipose | 0.0 |

TABLE YC
Panel 2D

| | Rel. | | Rel. |
|---|---|---|---|
| | Ep. (%) | | Exp. (%) |
| | Ag1757, | | Ag1757, |
| | Run | | Run |
| Tissue Name | 156016293 | Tissue Name | 156016293 |
| Normal Colon | 0.0 | Kidney Margin 8120608 | 0.0 |
| CC Well to Mod Diff (ODO3866) | 0.1 | Kidney Cancer 8120613 | 0.0 |
| CC Margin (ODO3866) | 0.0 | Kidney Margin 8120614 | 0.0 |
| CC Gr.2 rectosigmoid (ODO3868) | 0.0 | Kidney Cancer 9010320 | 0.0 |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon (ODO3921) | 0.0 | Normal Thyroid | 0.0 |
| CC Margin (ODO3921) | 0.0 | Thyroid Cancer 064010 | 0.0 |
| CC from Partial Hepatectomy | 0.1 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451-01) | 0.0 | Normal Breast | 0.0 |
| Lung Margin (OD04451-02) | 0.0 | Breast Cancer (OD04566) | 0.0 |
| Normal Prostate 6546-1 | 0.0 | Breast Cancer (OD04590-01) | 0.0 |
| Prostate Cancer (OD04410) | 0.0 | Breast Cancer Mets | 0.0 |
| | | (OD04590-03) | |
| Prostate Margin (OD04410) | 0.0 | Breast Cancer Metastasis | 0.0 |
| | | (OD04655-05) | |
| Prostate Cancer (OD04720-01) | 0.0 | Breast Cancer 064006 | 0.0 |
| Prostate Margin (OD04720-02) | 0.0 | Breast Cancer 1024 | 0.0 |
| Normal Lung 061010 | 0.0 | Breast Cancer 9100266 | 0.0 |
| Lung Met to Muscle (ODO4286) | 0.0 | Breast Margin 9100265 | 0.0 |
| Muscle Margin (ODO4286) | 0.0 | Breast Cancer A209073 | 0.0 |
| Lung Malignant Cancer (OD03126) | 0.0 | Breast Margin A209073 | 0.0 |
| Lung Margin (OD03126) | 0.0 | Normal Liver | 0.0 |
| Lung Cancer (OD04404) | 0.0 | Liver Cancer 064003 | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.0 | Liver Tissue 6004-N | 0.0 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 100.0 |
| Melanoma Mets to Lung | 0.0 | Bladder Cancer 1023 | 0.0 |
| (OD04321) | | | |
| Lung Margin (OD04321) | 0.0 | Bladder Cancer A302173 | 0.0 |
| Normal Kidney | 0.0 | Bladder Cancer (OD04718-01) | 0.0 |
| Kidney Ca, Nuclear grade 2 | 0.0 | Bladder Normal Adjacent | 0.0 |
| (OD04338) | | (OD04718-03) | |
| Kidney Margin (OD04338) | 0.0 | Normal Ovary | 0.0 |
| Kidney Ca Nuclear grade 1/2 | 0.0 | Ovarian Cancer 064008 | 0.0 |
| (OD04339) | | | |
| Kidney Margin (OD04339) | 0.0 | Ovarian Cancer (OD04768-07) | 0.0 |
| Kidney Ca, Clear cell type | 0.0 | Ovary Margin (OD04768-08) | 0.0 |
| (OD04340) | | | |
| Kidney Margin (OD04340) | 0.0 | Normal Stomach | 3.6 |
| Kidney Ca, Nuclear grade 3 | 0.0 | Gastric Cancer 9060358 | 0.0 |
| (OD04348) | | | |
| Kidney Margin (OD04348) | 0.0 | Stomach Margin 9060359 | 6.6 |
| Kidney Cancer (OD04622-01) | 0.0 | Gastric Cancer 9060395 | 0.0 |
| Kidney Margin (OD04622-03) | 0.0 | Stomach Margin 9060394 | 1.1 |
| Kidney Cancer (OD04450-01) | 0.0 | Gastric Cancer 9060397 | 0.0 |
| Kidney Margin (OD04450-03) | 0.0 | Stomach Margin 9060396 | 1.3 |
| Kidney Cancer 8120607 | 0.0 | Gastric Cancer 064005 | 0.3 |

TABLE YD
Panel 4D

| | Rel. | | Rel. |
|---|---|---|---|
| | xp. (%) | | Exp. (%) |
| | Ag1757, | | Ag1757, |
| | Run | | Run |
| Tissue Name | 156016294 | Tissue Name | 156016294 |
| Secondary Th1 act | 5.2 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + | 0.0 |
| | | IL-1beta | |
| Primary Th2 rest | 0.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte act | 0.0 | Coronery artery SMC rest | 2.6 |
| CD45RO CD4 lymphocyte act | 0.0 | Coronery artery SMC TNF alpha + | 0.0 |
| | | IL-1beta | |
| CD8 lymphocyte act | 0.0 | Astrocytes rest | 0.0 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 4.8 |
| Two Way MLR 3 day | 0.0 | NCI-H292 IFN gamma | 6.9 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 | 0.0 |
| PMA/ionomycin | | IL-1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 100.0 |
| Monocytes LPS | 0.0 | Colon | 96.6 |
| Macrophages rest | 0.0 | Lung | 0.0 |
| Macrophages LPS | 0.0 | Thymus | 0.0 |
| HUVEC none | 0.0 | Kidney | 54.0 |
| HUVEC starved | 0.0 | | |

TABLE YE
Panel 5 Islet

| | Rel. | Rel. |
|---|---|---|
| | Exp. (%) | Exp. (%) |
| | Ag1757 | Ag1757, |
| | Run | Run |
| Tissue Name | 172213986 | 242726292 |
| 97457_Patient-02go_adipose | 0.0 | 0.0 |
| 97476_Patient-07sk_skeletal | 0.0 | 0.0 |
| muscle | | |
| 97477_Patient-07ut_uterus | 0.0 | 0.0 |
| 97478_Patient-07pl_placenta | 0.0 | 0.0 |
| 99167_Bayer Patient 1 | 100.0 | 100.0 |
| 97482_Patient-08ut_uterus | 0.0 | 0.0 |
| 97483_Patient-08pl_placenta | 0.0 | 0.0 |
| 97486_Patient-09sk_skeletal | 0.0 | 0.0 |
| muscle | | |
| 97487_Patient-09ut_uterus | 0.0 | 0.0 |
| 97488_Patient-09pl_placenta | 0.0 | 0.0 |
| 97492_Patient-10ut_uterus | 0.0 | 0.0 |
| 97493_Patient-10pl_placenta | 0.0 | 0.0 |
| 97495_Patient-11go_adipose | 0.0 | 0.0 |
| 97496_Patient-11sk_skeletal | 0.0 | 0.0 |
| muscle | | |
| 97497_Patient-11ut_uterus | 0.0 | 0.0 |
| 97498_Patient-11pl_placenta | 0.0 | 0.0 |
| 97500_Patient-12go_adipose | 0.0 | 0.0 |
| 97501_Patient-12sk_skeletal | 0.0 | 0.0 |
| muscle | | |
| 97502_Patient-12ut_uterus | 0.0 | 0.0 |
| 97503_Patient-12pl_placenta | 0.0 | 0.0 |
| 94721_Donor 2 U - A_Mesenchymal | 0.0 | 0.0 |
| Stem Cells | | |
| 94722_Donor 2 U - B_Mesenchymal | 0.0 | 0.0 |
| Stem Cells | | |
| 94723_Donor 2 U - C_Mesenchymal | 0.0 | 0.0 |
| Stem Cells | | |
| 94709_Donor 2 AM - A_adipose | 0.0 | 0.0 |
| 94710_Donor 2 AM - B_adipose | 0.0 | 0.0 |
| 94711_Donor 2 AM - C_adipose | 0.0 | 0.0 |
| 94712_Donor 2 AD - A_adipose | 0.0 | 0.0 |
| 94713_Donor 2 AD - B_adipose | 0.0 | 0.0 |
| 94714_Donor 2 AD - C_adipose | 0.0 | 0.0 |
| 94742_Donor 3 U - A_Mesenchymal | 0.0 | 0.0 |
| Stem Cells | | |
| 94743_Donor 3 U - B_Mesenchymal | 0.0 | 0.0 |
| Stem Cells | | |
| 94730_Donor 3 AM - A_adipose | 0.0 | 0.0 |
| 94731_Donor 3 AM - B_adipose | 0.0 | 0.0 |
| 94732_Donor 3 AM - C_adipose | 0.0 | 0.0 |
| 94733_Donor 3 AD - A_adipose | 0.0 | 0.0 |
| 94734_Donor 3 AD - B_adipose | 0.0 | 0.0 |
| 94735_Donor 3 AD - C_adipose | 0.0 | 0.0 |
| 77138_Liver_HepG2untreated | 0.0 | 0.0 |
| 73556_Heart_Cardiac stromal cells | 0.0 | 0.0 |
| (primary) | | |
| 81735_Small Intestine | 0.0 | 0.0 |
| 72409_Kidney_Proximal Convoluted | 0.0 | 0.0 |
| Tubule | | |
| 82685_Small intestine_Duodenum | 0.0 | 0.1 |
| 90650_Adrenal_Adrenocortical | 0.0 | 0.0 |
| adenoma | | |
| 72410_Kidney_HRCE | 0.0 | 0.0 |
| 72411_Kidney_HRE | 0.0 | 0.0 |
| 73139_Uterus_Uterine smooth muscle | 0.0 | 0.0 |
| cells | | |

Panel 1.3D Summary: Ag1757 Highest expression of this gene is detected in pancrease (CT=22.5). High expression of this gene is also seen in bladder and stomach, while low expression was detected in lung, and fetal skeletal muscle. This gene codes for carboxypeptidase A2 (CPA2). CPA2 was found to be up-regulated in the GeneCalling studies in the spontaneous hypertensive rat, a model for hyperlipidemia, diabetes, and cardiovascular disease, and was down-regulated after treatment with troglitazone. These data suggest that down-regulation of CPA2 and decreased proteolysis may be beneficial for insulin sensitivity. At the same time, down-regulation of enzymes involved in hormone maturation have been implicated in the development of the obese phenotype, suggesting that down-regulation of CPA2 via small molecule drug may be an effective treatment for obesity.

Panel 2D Summary: Ag1757 High expression of this gene is seen only normal bladder. Hence expression of this gene may be used to distinguish bladder from other samples in this panel.

Panel 4D Summary: Ag1757 Low expression of this gene is seen exclusively in IBD Crohn's and colon sample. Therefore, therapeutic modulation of this gene may be useful in the treatment of Crohn's disease.

Panel 5 Islet Summary: Ag1757 Two experiments with same probe-primer sets are in good agreement. High expression of this gene is restricted to pancreatic islet cells (CTs=25.9-26.7), which supports the finding on panel 1.3. This gene product may be related to the fed state and may thus be a satiety signal. Therapeutic modulation of this gene or its protein product produced by islet cells may induce satiety and be a treatment for obesity.

Z. CG57417-01: Human SERCA 1-Like Protein.

Expression of gene CG57417-01 was assessed using the primer-probe set Ag3267, described in Table ZA. Results of the RTQ-PCR runs are shown in Tables ZB, ZC, ZD, ZE, ZF, ZG, ZH, ZI and ZJ.

TABLE ZA

Probe Name Ag3267

| | | | |
|---|---|---|---|
| | | | SEQ ID |
| Primers | Length | Start Position | No |

| | | | | |
|---|---|---|---|---|
| Forward | 5′-ccctctcaaccttgtaaattccc-3′ | 23 | 3313 | 461 |
| Probe | TET-5′-ttgcagggacaaggcgaccga-3′-TAMRA | 21 | 3355 | 462 |
| Reverse | 5′-aataaataagcagctcagcgca-3′ | 22 | 3377 | 463 |

TABLE ZB
CNS_neurodegeneration_v1.0

| | Rel. | | Rel. |
|---|---|---|---|
| | Exp. (%) | | Exp. (%) |
| | Ag3267, | | Ag3267, |
| | Run | | Run |
| Tissue Name | 210038490 | Tissue Name | 210038490 |
| AD 1 Hippo | 16.0 | Control (Path) 3 Temporal Ctx | 10.4 |
| AD 2 Hippo | 10.8 | Control (Path) 4 Temporal Ctx | 43.8 |
| AD 3 Hippo | 59.0 | AD 1 Occipital Ctx | 17.6 |
| AD 4 Hippo | 5.8 | AD 2 Occipital Ctx (Missing) | 0.0 |
| AD 5 Hippo | 29.3 | AD 3 Occipital Ctx | 16.3 |
| AD 6 Hippo | 66.0 | AD 4 Occipital Ctx | 17.4 |
| Control 2 Hippo | 23.8 | AD 5 Occipital Ctx | 10.3 |
| Control 4 Hippo | 16.2 | AD 6 Occipital Ctx | 5.7 |
| Control (Path) 3 Hippo | 4.8 | Control 1 Occipital Ctx | 0.0 |
| AD 1 Temporal Ctx | 56.3 | Control 2 Occipital Ctx | 21.9 |
| AD 2 Temporal Ctx | 24.7 | Control 3 Occipital Ctx | 50.0 |
| AD 3 Temporal Ctx | 43.8 | Control 4 Occipital Ctx | 0.0 |
| AD 4 Temporal Ctx | 30.1 | Control (Path) 1 Occipital Ctx | 100.0 |
| AD 5 Inf Temporal Ctx | 74.7 | Control (Path) 2 Occipital Ctx | 36.1 |
| AD 5 Sup Temporal Ctx | 83.5 | Control (Path) 3 Occipital Ctx | 11.2 |
| AD 6 Inf Temporal Ctx | 58.2 | Control (Path) 4 Occipital Ctx | 80.7 |
| AD 6 Sup Temporal Ctx | 65.5 | Control 1 Parietal Ctx | 10.5 |
| Control 1 Temporal Ctx | 8.4 | Control 2 Parietal Ctx | 62.9 |
| Control 2 Temporal Ctx | 22.5 | Control 3 Parietal Ctx | 11.0 |
| Control 3 Temporal Ctx | 33.7 | Control (Path) 1 Parietal Ctx | 39.2 |
| Control 3 Temporal Ctx | 17.8 | Control (Path) 2 Parietal Ctx | 53.6 |
| Control (Path) 1 Temporal Ctx | 62.0 | Control (Path) 3 Parietal Ctx | 2.8 |
| Control (Path) 2 Temporal Ctx | 31.2 | Control (Path) 4 Parietal Ctx | 42.6 |

TABLE ZC
General screening panel v1.4

| | Rel. | Rel. |
|---|---|---|
| | Exp. (%) | Exp. (%) |
| | Ag3267, | Ag3267, |
| | Run | Run |
| Tissue Name | 208010012 | 212650192 |
| Adipose | 2.5 | 0.3 |
| Melanoma* Hs688(A).T | 0.0 | 0.1 |
| Melanoma* Hs688(B).T | 0.0 | 0.0 |
| Melanoma* M14 | 0.1 | 0.1 |
| Melanoma* LOXIMVI | 0.0 | 0.0 |
| Melanoma* SK-MEL-5 | 0.1 | 0.1 |
| Squamous cell carcinoma SCC-4 | 0.0 | 0.0 |
| Testis Pool | 0.2 | 0.1 |
| Prostate ca.* (bone met) PC-3 | 0.1 | 0.1 |
| Prostate Pool | 0.1 | 0.2 |
| Placenta | 0.1 | 0.1 |
| Uterus Pool | 0.0 | 0.0 |
| Ovarian ca. OVCAR-3 | 0.1 | 0.2 |
| Ovarian ca. SK-OV-3 | 0.2 | 0.3 |
| Ovarian ca. OVCAR-4 | 0.0 | 0.1 |
| Ovarian ca. OVCAR-5 | 0.3 | 0.3 |
| Ovarian ca. IGROV-1 | 0.0 | 0.0 |
| Ovarian ca. OVCAR-8 | 0.0 | 0.1 |
| Ovary | 0.0 | 0.1 |
| Breast ca. MCF-7 | 0.1 | 0.2 |
| Breast ca. MDA-MB-231 | 0.1 | 0.2 |
| Breast ca. BT 549 | 0.1 | 0.2 |
| Breast ca. T47D | 0.2 | 0.4 |
| Breast ca. MDA-N | 0.1 | 0.1 |
| Breast Pool | 0.1 | 0.1 |
| Trachea | 0.2 | 0.2 |
| Lung | 0.1 | 0.1 |
| Fetal Lung | 0.1 | 0.2 |
| Lung ca. NCI-N417 | 0.0 | 0.0 |
| Lung ca. LX-1 | 0.4 | 0.4 |
| Lung ca. NCI-H146 | 0.1 | 0.1 |
| Lung ca. SHP-77 | 0.2 | 0.3 |
| Lung ca. A549 | 0.1 | 0.2 |
| Lung ca. NCI-H526 | 0.0 | 0.0 |
| Lung ca. NCI-H23 | 0.2 | 0.3 |
| Lung ca. NCI-H460 | 0.1 | 0.1 |
| Lung ca. HOP-62 | 0.1 | 0.1 |
| Lung ca. NCI-H522 | 0.3 | 0.3 |
| Liver | 0.0 | 0.0 |
| Fetal Liver | 0.0 | 0.1 |
| Liver ca. HepG2 | 0.4 | 0.4 |
| Kidney Pool | 0.1 | 0.2 |
| Fetal Kidney | 0.3 | 0.2 |
| Renal ca. 786-0 | 0.1 | 0.1 |
| Renal ca. A498 | 0.1 | 0.1 |
| Renal ca. ACHN | 0.1 | 0.2 |
| Renal ca. UO-31 | 0.1 | 0.1 |
| Renal ca. TK-10 | 0.5 | 0.7 |
| Bladder | 0.3 | 0.3 |
| Gastric ca. (liver met.) NCI-N87 | 1.4 | 1.4 |
| Gastric ca. KATO III | 0.3 | 0.3 |
| Colon ca. SW-948 | 0.0 | 0.0 |
| Colon ca. SW480 | 0.1 | 0.2 |
| Colon ca.* (SW480 met) SW620 | 0.1 | 0.2 |
| Colon ca. HT29 | 0.1 | 0.1 |
| Colon ca. HCT-116 | 0.3 | 0.4 |
| Colon ca. CaCo-2 | 0.2 | 0.2 |
| Colon cancer tissue | 0.1 | 0.1 |
| Colon ca. SW1116 | 0.1 | 0.1 |
| Colon ca. Colo-205 | 0.0 | 0.1 |
| Colon ca. SW-48 | 0.0 | 0.1 |
| Colon Pool | 0.1 | 0.6 |
| Small Intestine Pool | 0.2 | 0.2 |
| Stomach Pool | 0.1 | 0.2 |
| Bone Marrow Pool | 0.0 | 0.0 |
| Fetal Heart | 0.1 | 0.1 |
| Heart Pool | 0.0 | 0.0 |
| Lymph Node Pool | 0.1 | 0.2 |
| Fetal Skeletal Muscle | 16.7 | 17.0 |
| Skeletal Muscle Pool | 100.0 | 100.0 |
| Spleen Pool | 0.2 | 0.2 |
| Thymus Pool | 0.1 | 0.2 |
| CNS cancer (glio/astro) U87-MG | 0.2 | 0.2 |
| CNS cancer (glio/astro) U-118-MG | 0.2 | 0.3 |
| CNS cancer (neuro; met) SK-N-AS | 0.2 | 0.4 |
| CNS cancer (astro) SF-539 | 0.1 | 0.1 |
| CNS cancer (astro) SNB-75 | 0.1 | 0.1 |
| CNS cancer (glio) SNB-19 | 0.0 | 0.0 |
| CNS cancer (glio) SF-295 | 0.3 | 0.6 |
| Brain (Amygdala) Pool | 0.0 | 0.0 |
| Brain (cerebellum) | 0.1 | 0.1 |
| Brain (fetal) | 0.2 | 0.2 |
| Brain (Hippocampus) Pool | 0.0 | 0.1 |
| Cerebral Cortex Pool | 0.0 | 0.1 |
| Brain (Substantia nigra) Pool | 0.0 | 0.0 |
| Brain (Thalamus) Pool | 0.1 | 0.1 |
| Brain (whole) | 0.1 | 0.1 |
| Spinal Cord Pool | 0.2 | 0.3 |
| Adrenal Gland | 0.1 | 0.1 |
| Pituitary gland Pool | 0.0 | 0.1 |
| Salivary Gland | 0.2 | 0.3 |
| Thyroid (female) | 0.0 | 0.0 |
| Pancreatic ca. CAPAN2 | 0.1 | 0.1 |
| Pancreas Pool | 0.2 | 0.1 |

TABLE ZD
General_screening_panel_v1.7

| | Rel. | | Rel. |
|---|---|---|---|
| | Exp. (%) | | Exp. (%) |
| | Ag3267, | | Ag3267, |
| | Run | | Run |
| Tissue Name | 317617205 | Tissue Name | 317617205 |
| Adipose | 6.9 | Gastric ca. (liver met.) NCI-N87 | 0.0 |
| HUVEC | 0.0 | Stomach | 0.0 |
| Melanoma* Hs688(A).T | 0.0 | Colon ca. SW-948 | 0.0 |
| Melanoma* Hs688(B).T | 0.0 | Colon ca. SW480 | 0.0 |
| Melanoma (met) SK-MEL-5 | 0.0 | Colon ca. (SW480 met) SW620 | 0.0 |
| Testis | 0.1 | Colon ca. HT29 | 0.1 |
| Prostate ca. (bone met) PC-3 | 0.0 | Colon ca. HCT-116 | 0.1 |
| Prostate ca. DU145 | 0.0 | Colon cancer tissue | 0.0 |
| Prostate pool | 0.0 | Colon ca. SW1116 | 0.1 |
| Uterus pool | 0.0 | Colon ca. Colo-205 | 0.0 |
| Ovarian ca. OVCAR-3 | 0.0 | Colon ca. SW-48 | 0.0 |
| Ovarian ca. (ascites) SK-OV-3 | 0.0 | Colon | 0.1 |
| Ovarian ca. OVCAR-4 | 0.0 | Small Intestine | 0.0 |
| Ovarian ca. OVCAR-5 | 0.0 | Fetal Heart | 0.0 |
| Ovarian ca. IGROV-1 | 0.0 | Heart | 0.0 |
| Ovarian ca. OVCAR-8 | 0.0 | Lymph Node pool 1 | 0.0 |
| Ovary | 0.0 | Lymph Node pool 2 | 0.3 |
| Breast ca. MCF-7 | 0.0 | Fetal Skeletal Muscle | 8.5 |
| Breast ca. MDA-MB-231 | 0.1 | Skeletal Muscle pool | 7.2 |
| Breast ca. BT-549 | 0.0 | Skeletal Muscle | 100.0 |
| Breast ca. T47D | 0.1 | Spleen | 0.1 |
| Breast pool | 0.0 | Thymus | 0.0 |
| Trachea | 0.3 | CNS cancer (glio/astro) SF-268 | 0.0 |
| Lung | 0.1 | CNS cancer (glio/astro) T98G | 0.0 |
| Fetal Lung | 0.1 | CNS cancer (neuro; met) SK-N-AS | 0.0 |
| Lung ca. NCI-N417 | 0.0 | CNS cancer (astro) SF-539 | 0.1 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.1 | CNS cancer (glio) SF-295 | 0.0 |
| Lung ca. NCI-H23 | 0.0 | Brain (Amygdala) | 0.0 |
| Lung ca. NCI-H460 | 0.1 | Brain (Cerebellum) | 0.0 |
| Lung ca. HOP-62 | 0.0 | Brain (Fetal) | 0.2 |
| Lung ca. NCI-H522 | 0.1 | Brain (Hippocampus) | 0.0 |
| Lung ca. DMS-114 | 0.0 | Cerebral Cortex pool | 0.0 |
| Liver | 0.0 | Brain (Substantia nigra) | 0.0 |
| Fetal Liver | 0.0 | Brain (Thalamus) | 0.0 |
| Kidney pool | 0.1 | Brain (Whole) | 0.2 |
| Fetal Kidney | 0.0 | Spinal Cord | 0.0 |
| Renal ca. 786-0 | 0.1 | Adrenal Gland | 0.1 |
| Renal ca. A498 | 0.0 | Pituitary Gland | 0.1 |
| Renal ca. ACHN | 0.1 | Salivary Gland | 0.2 |
| Renal ca. UO-31 | 0.0 | Thyroid | 1.2 |
| Renal ca. TK-10 | 0.1 | Pancreatic ca. PANC-1 | 0.0 |
| Bladder | 0.0 | Pancreas pool | 0.0 |

TABLE ZE
Panel 1.3D

| | Rel. | | Rel. |
|---|---|---|---|
| | Exp. %) | | Exp. (%) |
| | Ag3267, | | Ag3267, |
| | Run | | Run |
| Tissue Name | 165296240 | Tissue Name | 165296240 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 1.4 | Renal ca. ACHN | 0.0 |
| Salivary gland | 0.3 | Renal ca. UO-31 | 0.0 |
| Pituitary gland | 0.0 | Renal ca. TK-10 | 0.0 |
| Brain (fetal) | 0.1 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.1 | Lung | 0.0 |
| Brain (hippocampus) | 0.1 | Lung (fetal) | 0.0 |
| Brain (substantia nigra) | 0.0 | Lung ca. (small cell) LX-1 | 0.0 |
| Brain (thalamus) | 0.1 | Lung ca. (small cell) NCI-H69 | 0.0 |
| Cerebral Cortex | 0.0 | Lung ca. (s.cell var.) SHP-77 | 0.0 |
| Spinal cord | 0.1 | Lung ca. (large cell) NCI-H460 | 0.0 |
| glio/astro U87-MG | 0.0 | Lung ca. (non-sm. cell) A549 | 0.0 |
| glio/astro U-118-MG | 0.0 | Lung ca. (non-s.cell) NCI-H23 | 0.0 |
| astrocytoma SW1783 | 0.0 | Lung ca. (non-s.cell) HOP-62 | 0.0 |
| neuro*; met SK-N-AS | 0.0 | Lung ca. (non-s.cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) MDA-MB-231 | 0.0 |
| Heart (fetal) | 0.0 | Breast ca.* (pl.ef) T47D | 0.0 |
| Heart | 0.0 | Breast ca. BT-549 | 0.0 |
| Skeletal muscle (fetal) | 18.6 | Breast ca. MDA-N | 0.0 |
| Skeletal muscle | 100.0 | Ovary | 0.0 |
| Bone marrow | 0.3 | Ovarian ca. OVCAR-3 | 0.0 |
| Thymus | 0.0 | Ovarian ca. OVCAR-4 | 0.0 |
| Spleen | 0.1 | Ovarian ca. OVCAR-5 | 0.0 |
| Lymph node | 0.2 | Ovarian ca. OVCAR-8 | 0.0 |
| Colorectal | 0.0 | Ovarian ca. IGROV-1 | 0.0 |
| Stomach | 0.1 | Ovarian ca.* (ascites) SK-OV-3 | 0.0 |
| Small intestine | 0.0 | Uterus | 0.0 |
| Colon ca. SW480 | 0.0 | Placenta | 0.0 |
| Colon ca.* SW620 (SW480 met) | 0.0 | Prostate | 0.2 |
| Colon ca. HT29 | 0.0 | Prostate ca.* (bone met) PC-3 | 0.0 |
| Colon ca. HCT-116 | 0.0 | Testis | 0.5 |
| Colon ca. CaCo-2 | 0.0 | Melanoma Hs688(A).T | 0.0 |
| Colon ca. tissue (ODO3866) | 0.0 | Melanoma* (met) Hs688(B).T | 0.0 |
| Colon ca. HCC-2998 | 0.0 | Melanoma UACC-62 | 0.0 |
| Gastric ca.* (liver met) NCI-N87 | 0.1 | Melanoma M14 | 0.0 |
| Bladder | 0.0 | Melanoma LOX IMVI | 0.0 |
| Trachea | 0.1 | Melanoma* (met) SK-MEL-5 | 0.0 |
| Kidney | 0.0 | Adipose | 0.4 |

TABLE ZF
Panel 2.2

| | Rel. | | Rel. |
|---|---|---|---|
| | Ep. (%) | | Exp. (%) |
| | Ag3267, | | Ag3267, |
| | Run | | Run |
| Tissue Name | 173762691 | Tissue Name | 173762691 |
| Normal Colon | 4.7 | Kidney Margin (OD04348) | 7.3 |
| Colon cancer (OD06064) | 0.0 | Kidney malignant cancer | 6.8 |
| | | (OD06204B) | |
| Colon Margin (OD06064) | 0.0 | Kidney normal adjacent tissue | 1.5 |
| | | (OD06204E) | |
| Colon cancer (OD06159) | 0.0 | Kidney Cancer (OD04450-01) | 8.5 |
| Colon Margin (OD06159) | 3.2 | Kidney Margin (OD04450-03) | 4.6 |
| Colon cancer (OD06297-04) | 0.0 | Kidney Cancer 8120613 | 5.4 |
| Colon Margin (OD06297-05) | 4.4 | Kidney Margin 8120614 | 2.0 |
| CC Gr.2 ascend colon (ODO3921) | 1.8 | Kidney Cancer 9010320 | 3.3 |
| CC Margin (ODO3921) | 0.0 | Kidney Margin 9010321 | 2.7 |
| Colon cancer metastasis | 1.6 | Kidney Cancer 8120607 | 7.6 |
| (OD06104) | | | |
| Lung Margin (OD06104) | 1.6 | Kidney Margin 8120608 | 0.0 |
| Colon mets to lung (OD04451-01) | 4.1 | Normal Uterus | 3.4 |
| Lung Margin (OD04451-02) | 0.0 | Uterine Cancer 064011 | 0.0 |
| Normal Prostate | 9.2 | Normal Thyroid | 100.0 |
| Prostate Cancer (OD04410) | 2.1 | Thyroid Cancer 064010 | 0.0 |
| Prostate Margin (OD04410) | 3.7 | Thyroid Cancer A302152 | 7.3 |
| Normal Ovary | 3.8 | Thyroid Margin A302153 | 0.0 |
| Ovarian cancer (OD06283-03) | 3.4 | Normal Breast | 2.6 |
| Ovarian Margin (OD06283-07) | 3.3 | Breast Cancer (OD04566) | 8.5 |
| Ovarian Cancer 064008 | 17.4 | Breast Cancer 1024 | 36.3 |
| Ovarian cancer (OD06145) | 1.4 | Breast Cancer (OD04590-01) | 10.2 |
| Ovarian Margin (OD06145) | 0.0 | Breast Cancer Mets | 3.1 |
| | | (OD04590-03) | |
| Ovarian cancer (OD06455-03) | 2.3 | Breast Cancer Metastasis | 10.6 |
| | | (OD04655-05) | |
| Ovarian Margin (OD06455-07) | 3.0 | Breast Cancer 064006 | 12.3 |
| Normal Lung | 1.6 | Breast Cancer 9100266 | 7.4 |
| Invasive poor diff. lung adeno | 8.8 | Breast Margin 9100265 | 4.9 |
| (ODO4945-01 | | | |
| Lung Margin (ODO4945-03) | 4.5 | Breast Cancer A209073 | 0.0 |
| Lung Malignant Cancer | 0.0 | Breast Margin A2090734 | 8.0 |
| (OD03126) | | | |
| Lung Margin (OD03126) | 0.0 | Breast cancer (OD06083) | 16.2 |
| Lung Cancer (OD05014A) | 1.6 | Breast cancer node metastasis | 15.7 |
| | | (OD06083) | |
| Lung Margin (OD05014B) | 1.5 | Normal Liver | 5.1 |
| Lung cancer (OD06081) | 1.4 | Liver Cancer 1026 | 1.4 |
| Lung Margin (OD06081) | 1.6 | Liver Cancer 1025 | 4.7 |
| Lung Cancer (OD04237-01) | 0.2 | Liver Cancer 6004-T | 2.1 |
| Lung Margin (OD04237-02) | 1.7 | Liver Tissue 6004-N | 11.8 |
| Ocular Melanoma Metastasis | 1.4 | Liver Cancer 6005-T | 0.0 |
| Ocular Melanoma Margin (Liver) | 0.0 | Liver Tissue 6005-N | 0.0 |
| Melanoma Metastasis | 0.0 | Liver Cancer 064003 | 0.0 |
| Melanoma Margin (Lung) | 0.0 | Normal Bladder | 6.7 |
| Normal Kidney | 4.6 | Bladder Cancer 1023 | 6.4 |
| Kidney Ca, Nuclear grade 2 | 10.9 | Bladder Cancer A302173 | 3.0 |
| (OD04338) | | | |
| Kidney Margin (OD04338) | 1.4 | Normal Stomach | 9.3 |
| Kidney Ca Nuclear grade 1/2 | 11.0 | Gastric Cancer 9060397 | 0.0 |
| (OD04339) | | | |
| Kidney Margin (OD04339) | 3.8 | Stomach Margin 9060396 | 0.7 |
| Kidney Ca, Clear cell type | 0.0 | Gastric Cancer 9060395 | 7.4 |
| (OD04340) | | | |
| Kidney Margin (OD04340) | 3.9 | Stomach Margin 9060394 | 3.5 |
| Kidney Ca, Nuclear grade 3 | 0.0 | Gastric Cancer 064005 | 3.5 |
| (OD04348) | | | |

TABLE ZG
Panel 3D

| | Rel. | | Rel. |
|---|---|---|---|
| | Ex. (%) | | Exp. (%) |
| | Ag3267, | | Ag3267, |
| | Run | | Run |
| Tissue Name | 165465032 | Tissue Name | 165465032 |
| Daoy- Medulloblastoma | 0.0 | Ca Ski- Cervical epidermoid | 0.0 |
| | | carcinoma (metastasis) | |
| TE671-Medulloblastoma | 100.0 | ES-2- Ovarian clear cell carcinoma | 0.0 |
| D283 Med- Medulloblastoma | 4.8 | Ramos- Stimulated with | 0.0 |
| | | PMA/ionomycin 6 h | |
| PFSK-1- Primitive | 1.7 | Ramos- Stimulated with | 2.7 |
| Neuroectodermal | | PMA/ionomycin 14 h | |
| XF-498- CNS | 0.6 | MEG-01- Chronic myelogenous | 1.8 |
| | | leukemia (megokaryoblast) | |
| SNB-78- Glioma | 1.5 | Raji- Burkitt's lymphoma | 8.5 |
| SF-268- Glioblastoma | 0.0 | Daudi- Burkitt's lymphoma | 7.3 |
| T98G- Glioblastoma | 0.0 | U266- B-cell plasmacytoma | 8.3 |
| SK-N-SH- Neuroblastoma | 1.8 | CA46- Burkitt's lymphoma | 1.7 |
| (metastasis) | | | |
| SF-295- Glioblastoma | 3.7 | RL-non-Hodgkin's B-cell | 0.6 |
| | | lymphoma | |
| Cerebellum | 0.7 | JM1- pre-B-cell lymphoma | 2.7 |
| Cerebellum | 2.7 | Jurkat- T cell leukemia | 2.2 |
| NCI-H292- Mucoepidermoid | 1.8 | TF-1- Erythroleukemia | 1.2 |
| lung carcinoma | | | |
| DMS-114- Small cell lung | 1.8 | HUT 78- T-cell lymphoma | 2.0 |
| cancer | | | |
| DMS-79- Small cell lung cancer | 6.6 | U937- Histiocytic lymphoma | 0.0 |
| NCI-H146- Small cell lung | 9.0 | KU-812- Myelogenous leukemia | 0.6 |
| cancer | | | |
| NCI-H526- Small cell lung | 2.1 | 769-P- Clear cell renal carcinoma | 0.0 |
| cancer | | | |
| NCI-N417- Small cell lung | 0.0 | Caki-2- Clear cell renal carcinoma | 2.2 |
| cancer | | | |
| NCI-H82- Small cell lung cancer | 5.3 | SW 839- Clear cell renal carcinoma | 0.5 |
| NCI-H157- Squamous cell lung | 0.0 | Rhabdoid kidney tumor | 4.0 |
| cancer (metastasis) | | | |
| NCI-H1155- Large cell lung | 7.2 | Hs766T- Pancreatic carcinoma(LN | 0.5 |
| cancer | | metastasis) | |
| NCI-H1299- Large cell lung | 1.3 | CAPAN-1- Pancreatic | 1.4 |
| cancer | | adenocarcinoma (liver metastasis) | |
| NCI-H727- Lung carcinoid | 4.6 | SU86.86- Pancreatic carcinoma | 1.5 |
| | | (liver metastasis) | |
| NCI-UMC-11- Lung carcinoid | 7.0 | BxPC-3- Pancreatic | 1.7 |
| | | adenocarcinoma | |
| LX-1- Small cell lung cancer | 1.6 | HPAC- Pancreatic adenocarcinoma | 0.6 |
| Colo-205- Colon cancer | 5.0 | MIA PaCa-2- Pancreatic carcinoma | 0.0 |
| KM12- Colon cancer | 0.0 | CFPAC-1- Pancreatic ductal | 5.7 |
| | | adenocarcinoma | |
| KM20L2- Colon cancer | 1.1 | PANC-1- Pancreatic epithelioid | 0.1 |
| | | ductal carcinoma | |
| NCI-H716- Colon cancer | 1.7 | T24- Bladder carcinma (transitional | 1.7 |
| | | cell) | |
| SW-48- Colon adenocarcinoma | 2.0 | 5637- Bladder carcinoma | 0.0 |
| SW1116- Colon adenocarcinoma | 3.1 | HT-1197- Bladder carcinoma | 1.4 |
| LS 174T- Colon adenocarcinoma | 1.3 | UM-UC-3- Bladder carcinma | 0.6 |
| | | (transitional cell) | |
| SW-948- Colon adenocarcinoma | 0.0 | A204- Rhabdomyosarcoma | 0.4 |
| SW-480- Colon adenocarcinoma | 1.6 | HT-1080- Fibrosarcoma | 2.7 |
| NCI-SNU-5- Gastric carcinoma | 0.3 | MG-63- Osteosarcoma | 0.0 |
| KATO III- Gastric carcinoma | 1.4 | SK-LMS-1- Leiomyosarcoma | 4.6 |
| | | (vulva) | |
| NCI-SNU-16- Gastric carcinoma | 4.1 | SJRH30- Rhabdomyosarcoma (met | 22.8 |
| | | to bone marrow) | |
| NCI-SNU-1- Gastric carcinoma | 2.3 | A431- Epidermoid carcinoma | 0.8 |
| RF-1- Gastric adenocarcinoma | 3.1 | WM266-4- Melanoma | 0.0 |
| RF-48- Gastric adenocarcinoma | 3.8 | DU 145- Prostate carcinoma (brain | 0.0 |
| | | metastasis) | |
| MKN-45- Gastric carcinoma | 6.3 | MDA-MB-468- Breast | 1.1 |
| | | adenocarcinoma | |
| NCI-N87- Gastric carcinoma | 2.0 | SCC-4- Squamous cell carcinoma of | 0.0 |
| | | tongue | |
| OVCAR-5- Ovarian carcinoma | 0.0 | SCC-9- Squamous cell carcinoma of | 0.0 |
| | | tongue | |
| RL95-2- Uterine carcinoma | 0.6 | SCC-15- Squamous cell carcinoma | 0.0 |
| | | of tongue | |
| HelaS3- Cervical | 1.3 | CAL 27- Squamous cell carcinoma | 3.1 |
| adenocarcinoma | | of tongue | |

TABLE ZH
Panel 4.1D

| | Rel. | | Rel. |
|---|---|---|---|
| | Exp. () | | Exp. (%) |
| | Ag3267, | | Ag3267, |
| | Run | | Run |
| Tissue Name | 169828983 | Tissue Name | 169828983 |
| Secondary Th1 act | 20.0 | HUVEC IL-1beta | 9.0 |
| Secondary Th2 act | 70.7 | HUVEC IFN gamma | 19.5 |
| Secondary Tr1 act | 53.2 | HUVEC TNF alpha + IFN gamma | 9.3 |
| Secondary Th1 rest | 8.0 | HUVEC TNF alpha + IL4 | 28.1 |
| Secondary Th2 rest | 27.9 | HUVEC IL-11 | 13.0 |
| Secondary Tr1 rest | 20.9 | Lung Microvascular EC none | 41.8 |
| PrimaryTh1 act | 33.4 | Lung Microvascular EC TNF alpha + | 16.0 |
| | | IL-1beta | |
| Primary Th2 act | 20.7 | Microvascular Dermal EC none | 13.8 |
| Primary Tr1 act | 22.5 | Microsvasular Dermal EC | 10.7 |
| | | TNF alpha + IL-1beta | |
| Primary Th1 rest | 14.2 | Bronchial epithelium TNF alpha + | 21.9 |
| | | IL-1beta | |
| Primary Th2 rest | 17.2 | Small airway epithelium none | 3.9 |
| Primary Tr1 rest | 23.0 | Small airway epithelium TNF alpha + | 8.7 |
| | | IL-1beta | |
| CD45RA CD4 lymphocyte act | 38.7 | Coronery artery SMC rest | 16.6 |
| CD45RO CD4 lymphocyte act | 42.6 | Coronery artery SMC TNF alpha + | 0.0 |
| | | IL-1beta | |
| CD8 lymphocyte act | 77.4 | Astrocytes rest | 5.6 |
| Secondary CD8 lymphocyte rest | 46.7 | Astrocytes TNF alpha + IL-1beta | 9.9 |
| Secondary CD8 lymphocyte act | 37.4 | KU-812 (Basophil) rest | 19.5 |
| CD4 lymphocyte none | 7.3 | KU-812 (Basophil) | 19.3 |
| | | PMA/ionomycin | |
| 2ry Th1/Th2/Tr1_anti-CD95 | 23.8 | CCD1106 (Keratinocytes) none | 41.2 |
| CH11 | | | |
| LAK cells rest | 41.2 | CCD1106 (Keratinocytes) | 0.0 |
| | | TNF alpha + IL-1beta | |
| LAK cells IL-2 | 46.0 | Liver cirrhosis | 16.3 |
| LAK cells IL-2 + IL-12 | 29.9 | NCI-H292 none | 41.8 |
| LAK cells IL-2 + IFN gamma | 45.4 | NCI-H292 IL-4 | 33.7 |
| LAK cells IL-2 + IL-18 | 52.5 | NCI-H292 IL-9 | 59.0 |
| LAK cells PMA/ionomycin | 37.6 | NCI-H292 IL-13 | 17.8 |
| NK Cells IL-2 rest | 31.2 | NCI-H292 IFN gamma | 41.5 |
| Two Way MLR 3 day | 36.6 | HPAEC none | 21.2 |
| Two Way MLR 5 day | 10.6 | HPAEC TNF alpha + IL-1beta | 14.7 |
| Two Way MLR 7 day | 25.7 | Lung fibroblast none | 19.2 |
| PBMC rest | 3.7 | Lung fibroblast TNF alpha + | 19.8 |
| | | IL-1beta | |
| PBMC PWM | 38.7 | Lung fibroblast IL-4 | 17.9 |
| PBMC PHA-L | 38.7 | Lung fibroblast IL-9 | 17.0 |
| Ramos (B cell) none | 49.7 | Lung fibroblast IL-13 | 14.0 |
| Ramos (B cell) ionomycin | 38.7 | Lung fibroblast IFN gamma | 0.0 |
| B lymphocytes PWM | 16.6 | Dermal fibroblast CCD1070 rest | 24.7 |
| B lymphocytes CD40L and IL-4 | 100.0 | Dermal fibroblast CCD1070 TNF | 44.4 |
| | | alpha | |
| EOL-1 dbcAMP | 40.6 | Dermal fibroblast CCD1070 | 9.2 |
| | | IL-1beta | |
| EOL-1 dbcAMP | 19.3 | Dermal fibroblast IFN gamma | 6.7 |
| PMA/ionomycin | | | |
| Dendritic cells none | 31.6 | Dermal fibroblast IL-4 | 17.2 |
| Dendritic cells LPS | 15.0 | Dermal Fibroblasts rest | 12.3 |
| Dendritic cells anti-CD40 | 34.2 | Neutrophils TNF a + LPS | 3.1 |
| Monocytes rest | 23.5 | Neutrophils rest | 0.0 |
| Monocytes LPS | 20.9 | Colon | 46.0 |
| Macrophages rest | 18.4 | Lung | 5.3 |
| Macrophages LPS | 0.0 | Thymus | 62.0 |
| HUVEC none | 12.5 | Kidney | 48.3 |
| HUVEC starved | 18.3 | | |

TABLE ZI
Panel 5 Islet

| | Rel. | | Rel. |
|---|---|---|---|
| | Exp. () | | Exp. (%) |
| | Ag3267, | | Ag3267, |
| | Run | | Run |
| Tissue Name | 172203503 | Tissue Name | 172203503 |
| 97457_Patient-02go_adipose | 0.0 | 94709_Donor 2 AM - A_adipose | 0.0 |
| 97476_Patient-07sk_skeletal | 6.9 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placenta | 0.0 | 94712_Donor 2 AD - A_adipose | 0.0 |
| 99167_Bayer Patient 1 | 0.0 | 94713_Donor 2 AD - B_adipose | 0.0 |
| 97482_Patient-08ut_uterus | 0.0 | 94714_Donor 2 AD - C_adipose | 0.0 |
| 97483_Patient-08pl_placenta | 0.0 | 94742_Donor 3 U - A_Mesenchymal | 0.0 |
| | | Stem Cells | |
| 97486_Patient-09sk_skeletal | 6.2 | 94743_Donor 3 U - B_Mesenchymal | 0.0 |
| muscle | | Stem Cells | |
| 97487_Patient-09ut_uterus | 0.0 | 94730_Donor 3 AM - A_adipose | 0.0 |
| 97488_Patient-09pl_placenta | 0.0 | 94731_Donor 3 AM - B_adipose | 0.0 |
| 97492_Patient-10ut_uterus | 0.0 | 94732_Donor 3 AM - C_adipose | 0.0 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal | 21.0 | 94735_Donor 3 AD - C_adipose | 0.0 |
| muscle | | | |
| 97497_Patient-11ut_uterus | 0.0 | 77138_Liver_HepG2untreated | 0.1 |
| 97498_Patient-11pl_placenta | 0.0 | 73556_Heart_Cardiac stromal cells | 0.0 |
| | | (primary) | |
| 97500_Patient-12go_adipose | 0.1 | 81735_Small Intestine | 0.0 |
| 97501_Patient-12sk_skeletal | 100.0 | 72409_Kidney_Proximal Convoluted | 0.0 |
| muscle | | Tubule | |
| 97502_Patient-12ut_uterus | 0.0 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2 U - | 0.0 | 72410_Kidney_HRCE | 0.1 |
| A_Mesenchymal Stem Cells | | | |
| 94722_Donor 2 U - | 0.0 | 72411_Kidney_HRE | 0.0 |
| B_Mesenchymal Stem Cells | | | |
| 94723_Donor 2 U - | 0.0 | 73139_Uterus_Uterine smooth | 0.0 |
| C_Mesenchymal Stem Cells | | muscle cells | |

TABLE ZJ
Panel 5D

| | Rel. | | Rel. |
|---|---|---|---|
| | Ex. (%) | | Exp. (%) |
| | Ag3267, | | Ag3267, |
| | Run | | Run |
| Tissue Name | 166510707 | Tissue Name | 166510707 |
| 97457_Patient-02go_adipose | 0.0 | 94709_Donor 2 AM - A_adipose | 0.0 |
| 97476_Patient-07sk_skeletal | 6.1 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placenta | 0.0 | 94712_Donor 2 AD - A_adipose | 0.0 |
| 97481_Patient-08sk_skeletal | 5.3 | 94713_Donor 2 AD - B_adipose | 0.0 |
| muscle | | | |
| 97482_Patient-08ut_uterus | 0.0 | 94714_Donor 2 AD - C_adipose | 0.0 |
| 97483_Patient-08pl_placenta | 0.0 | 94742_Donor 3 U - A_Mesenchymal | 0.0 |
| | | Stem Cells | |
| 97486_Patient-09sk_skeletal | 7.4 | 94743_Donor 3 U - B_Mesenchymal | 0.1 |
| muscle | | Stem Cells | |
| 97487_Patient-09ut_uterus | 0.0 | 94730_Donor 3 AM - A_adipose | 0.0 |
| 97488_Patient-09pl_placenta | 0.1 | 94731_Donor 3 AM - B_adipose | 0.1 |
| 97492_Patient-10ut_uterus | 0.0 | 94732_Donor 3 AM - C_adipose | 0.1 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal | 20.6 | 94735_Donor 3 AD - C_adipose | 0.0 |
| muscle | | | |
| 97497_Patient-11ut_uterus | 0.2 | 77138_Liver_HepG2untreated | 0.2 |
| 97498_Patient-11pl_placenta | 0.0 | 73556_Heart_Cardiac stromal cells | 0.0 |
| | | (primary) | |
| 97500_Patient-12go_adipose | 0.0 | 81735_Small Intestine | 0.1 |
| 97501_Patient-12sk_skeletal | 100.0 | 72409_Kidney_Proximal Convoluted | 0.0 |
| muscle | | Tubule | |
| 97502_Patient-12ut_uterus | 0.0 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2 U - | 0.0 | 72410_Kidney_HRCE | 0.0 |
| A_Mesenchymal Stem Cells | | | |
| 94722_Donor 2 U - | 0.0 | 72411_Kidney_HRE | 0.1 |
| B_Mesenchymal Stem Cells | | | |
| 94723_Donor 2 U - | 0.0 | 73139_Uterus_Uterine smooth | 0.0 |
| C_Mesenchymal Stem Cells | | muscle cells | |

CNS_neurodegeneration_v1.0 Summary: Ag3267 This panel confirms the expression of this gene at low levels in the brains of an independent group of individuals. Please see Panel 1.4 for a discussion of this gene in treatment of central nervous system disorders.

General_screening_panel_v1.4 Summary: Ag3267 Two experiments with same probe-primer sets are in excellent agreement. Highest expression of this gene is detected in skeletal muscle (CTs=21-22.7). Interestingly, expression of this gene is higher in adult compared to to fetal skeletal muscle. Therefore, expression of this gene may be used to distinguish adult skeletal muscle from fetal tissue and also other samples used in this panel.

This gene codes for SERCA1, a magnesium dependent enzyme that catalyzes the hydrolysis of ATP coupled with the translocation of calcium from the cytosol to the sarcoplasmic reticulum lumen. It contributes to calcium sequestration involved in muscular excitation/contraction. SERCA 1 is an integral membrane protein of the sarcoplasmic and endoplasmic reticulum and has 2 alternative spliced isoforms, serca1a/atp2a1a/adult and serca1b/atp2a1b/neonatal. The SERCA1 adult isoform accounts for more than 99% of serca1 expressed in adult, while isoform serca1b predominates in neo-natal fibers. Defects in atp2a1 are associated with some forms of the autosomal recessive inheritance of the Brody disease (bd), characterized by increasing impairment of relaxation of fast twist skeletal muscle during exercise. In addition, at Curagen it was found that in the muscle of the lean Cast/Ei mouse there was a mutation in SERCA1 which ablates its ATPase activity. The presence of a nonfunctional SERCA1 may lead to increased futile cycling of calcium, which may result in a leaner phenotype of these animals. Thus, an antagonist for SERCA1 may increase futile cycling and energy expenditure and could be beneficial in the treatment of obesity. On the other hand, increased activity of SERCA1 will replenish the calcium pool for adequate excitation-contraction coupling, leading to a better exercise-dependent insulin sensitivity of the muscle. Therefore, an agonist of SERCA1 could be beneficial for the treatment of diabetes.

This gene also shows low but ubiquitous expression in this panel, with moderate to low expression also seen in all the regions of brain, including including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Missiaen L, Robberecht W, van den Bosch L, Callewaert G, Parys J B, Wuytack F, Raeymaekers L, Nilius B, Eggermont J, De Smedt H. Abnormal intracellular ca(2+)homeostasis and disease. Cell Calcium. July 2000;28(1):1-21. Review.PMID: 10942700; Odermatt A, Barton K, Khanna V K, Mathieu J, Escolar D, Kuntzer T, Karpati G, MacLennan D H. The mutation of Pro789 to Leu reduces the activity of the fast-twitch skeletal muscle sarco(endo)plasmic reticulum Ca2+ ATPase (SERCA1) and is associated with Brody disease. HumGenet.May 2000;106(5):482-91. PMID: 10914677; Algenstaedt P, Antonetti D A, Yaffe M B, Kahn C R. Insulin receptor substrate proteins create a link between the tyrosine phosphorylation cascade and the Ca2+-ATPases in muscle and heart. J Biol Chem. Sep. 19, 1997;272(38):23696-702. PMID: 9295312; Thelen M H, Muller A, Zuidwijk M J, van der Linden G C, Simonides W S, van Hardeveld C. Differential regulation of the expression of fast-type sarcoplasmic-reticulum Ca(2+)-ATPase by thyroid hormone and insulin-like growth factor-I in the L6 muscle cell line.Biochem J. Oct. 15, 1994;303 (Pt 2):467-74. PMID: 7980406.

General_screening_panel_v1.7 Summary: Ag3267 Highest expression of this gene is detected in skeletal muscle (CTs=20). The expression profile in this panel correlates with that seen in panel 1.4. Please see panel 1.4 for further discussion of this gene.

Panel 1.3D Summary: Ag3267 Highest expression of this gene is detected in skeletal muscle (CTs=21.4). The expression profile in this panel correlates with that seen in panel 1.4. Please see panel 1.4 for further discussion on the utility of this gene.

Panel 2.2 Summary: Ag3267 Highest expression of this gene is detected in normal thyroid (CT=30.6). Low expression of this gene is also seen in gastric, bladder, breast, thyroid, kidney and ovarian cancers. Therefore, therapeutic modulation of this gene may be useful in the treatment of gastric, bladder, breast, thyroid, kidney and ovarian cancers.

Panel 3D Summary: Ag3267 Highest expression of this gene is detected in medulloblastoma cell line (CT=29). Moderate to low expression of this gene is seen in number of cell lines derived from tongue, bone, pancreatic, lymphoma, renal, gastric, colon, lung and brain cancers. Therefore, therapeutic modulation of this gene or its protein product may be useful in the treatment of tongue, bone, pancreatic, lymphoma, renal, gastric, colon, lung and brain cancers.

Panel 4.1D Summary: Ag3267 Highest expression of this gene is detected in CD40L and IL-4 treated B lymphocytes (CT=32.8). This gene show low expression in a wide range of cell types of significance in the immune response in health and disease. These cells include members of the T-cell, B-cell, endothelial cell, monocyte, and activated peripheral blood mononuclear cell family, as well as normal tissues represented by colon, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag3267 Moderate to high expression of this gene is restricted to skeletal muscle from diabetic and non-diabetic patients (CTs=26-30.3). Please see panel 1.4 for further discussion of this gene.

Panel 5D Summary: Ag3267 Moderate to high expression of this gene is restricted to skeletal muscle from diabetic and non-diabetic patients (CTs=25-29.3). Please see panel 1.4 for further discussion of this gene.

AA. CG93541-01: Human Autotaxin-t-Like (atx-t) Protein.

Expression of gene CG93541-01 was assessed using the primer-probe set Ag3857, described in Table AAA. Results of the RTQ-PCR runs are shown in Tables AAB, AAC, AAD, AAE, AAF and AAG.

TABLE AAA

Probe Name AG3857

| | | | |
|---|---|---|---|
| | | | SEQ ID |
| Primers | Length | Start Position | No |

| | | | | |
|---|---|---|---|---|
| Forward | 5′-tgcctggaactctaggaagaat-3′ | 22 | 1216 | 464 |
| Probe | TET-5′-tcgatccaaatttagcaacaatgcta-3′-TAMRA | 26 | 1238 | 465 |
| Reverse | 5′-agattggcaataatggctttg-3′ | 21 | 1274 | 466 |

TABLE AAB
CNS neurodegeneration v1.0

| | Rel. Exp.(%) |
|---|---|
| | Ag3857, Run |
| Tissue Name | 212187599 |
| AD 1 Hippo | 16.8 |
| AD 2 Hippo | 28.3 |
| AD 3 Hippo | 9.5 |
| AD 4 Hippo | 8.1 |
| AD 5 hippo | 36.9 |
| AD 6 Hippo | 59.5 |
| Control 2 Hippo | 49.3 |
| Control 4 Hippo | 33.9 |
| Control (Path) 3 Hippo | 46.3 |
| AD 1 Temporal Ctx | 31.2 |
| AD 2 Temporal Ctx | 26.4 |
| AD 3 Temporal Ctx | 5.1 |
| AD 4 Temporal Ctx | 26.4 |
| AD 5 Inf Temporal Ctx | 81.8 |
| AD 5 Sup Temporal Ctx | 90.1 |
| AD 6 Inf Temporal Ctx | 59.0 |
| AD 6 Sup Temporal Ctx | 36.6 |
| Control 1 Temporal Ctx | 3.9 |
| Control 2 Temporal Ctx | 40.1 |
| Control 3 Temporal Ctx | 11.6 |
| Control 4 Temporal Ctx | 6.4 |
| Control (Path) 1 Temporal Ctx | 30.6 |
| Control (Path) 2 Temporal Ctx | 12.9 |
| Control (Path) 3 Temporal Ctx | 1.6 |
| Control (Path) 4 Temporal Ctx | 11.3 |
| AD 1 Occipital Ctx | 19.1 |
| AD 2 Occipital Ctx (Missing) | 0.0 |
| AD 3 Occipital Ctx | 8.7 |
| AD 4 Occipital Ctx | 26.1 |
| AD 5 Occipital Ctx | 14.7 |
| AD 6 Occipital Ctx | 47.3 |
| Control 1 Occipital Ctx | 4.1 |
| Control 2 Occipital Ctx | 62.9 |
| Control 3 Occipital Ctx | 10.4 |
| Control 4 Occipital Ctx | 13.7 |
| Control (Path) 1 Occipital Ctx | 100.0 |
| Control (Path) 2 Occipital Ctx | 15.1 |
| Control (Path) 3 Occipital Ctx | 4.0 |
| Control (Path) 4 Occipital Ctx | 10.4 |
| Control 1 Parietal Ctx | 10.2 |
| Control 2 Parietal Ctx | 38.4 |
| Control 3 Parietal Ctx | 19.6 |
| Control (Path) 1 Parietal Ctx | 38.4 |
| Control (Path) 2 Parietal Ctx | 20.3 |
| Control (Path) 3 Parietal Ctx | 1.5 |
| Control (Path) 4 Parietal Ctx | 21.0 |

TABLE AAC
General screening panel v1.5

| | Rel. Exp.(%) |
|---|---|
| | Ag3857, Run |
| Tissue Name | 244371052 |
| Adipose | 15.5 |
| Melanoma* Hs688(A).T | 44.4 |
| Melanoma* Hs688(B).T | 8.7 |
| Melanoma* M14 | 5.9 |
| Melanoma* LOXIMVI | 0.2 |
| Melanoma* SK-MEL-5 | 0.1 |
| Squamous Cell carcinoma SCC-4 | 0.0 |
| Testis Pool | 6.4 |
| Prostate ca.* (bone met) PC-3 | 0.2 |
| Prostate Pool | 8.5 |
| Placenta | 9.6 |
| Uterus Pool | 28.3 |
| Ovarian ca. OVCAR-3 | 0.1 |
| Ovarian ca. SK-OV-3 | 4.2 |
| Ovarian ca. OVCAR-4 | 0.1 |
| Ovarian ca. OVCAR-5 | 0.0 |
| Ovarian ca. IGROV-1 | 0.0 |
| Ovarian ca. OVCAR-8 | 0.0 |
| Ovary | 12.3 |
| Breast ca. MCF-7 | 0.1 |
| Breast ca. MDA-MB-231 | 0.2 |
| Breast ca. BT 549 | 3.5 |
| Breast ca. T47D | 0.0 |
| Breast ca. MDA-N | 6.7 |
| Breast Pool | 40.1 |
| Trachea | 2.4 |
| Lung | 1.7 |
| Fetal Lung | 41.8 |
| Lung ca. NCI-N417 | 0.0 |
| Lung ca. LX-1 | 0.6 |
| Lung ca. NCI-H146 | 0.0 |
| Lung ca. SHP-77 | 0.1 |
| Lung ca. A549 | 0.0 |
| Lung ca. NCI-H526 | 0.0 |
| Lung ca. NCI-H23 | 0.1 |
| Lung ca. NCI-H460 | 0.2 |
| Lung ca. HOP-62 | 0.7 |
| Lung ca. NCI-H522 | 0.1 |
| Liver | 0.4 |
| Fetal Liver | 17.6 |
| Liver ca. HepG2 | 0.2 |
| Kidney Pool | 21.0 |
| Fetal Kidney | 8.8 |
| Renal ca. 786-0 | 0.0 |
| Renal ca. A498 | 1.5 |
| Renal ca. ACHN | 0.3 |
| Renal ca. UO-31 | 0.0 |
| Renal ca. TK-10 | 0.2 |
| Bladder | 7.0 |
| Gastric ca. (liver met.) NCI-N87 | 0.2 |
| Gastric ca. KATO III | 0.0 |
| Colon ca. SW-948 | 0.0 |
| Colon ca. SW480 | 0.1 |
| Colon ca* (SW480 met) SW620 | 0.0 |
| Colon ca. HT29 | 0.1 |
| Colon ca. HCT-116 | 0.0 |
| Colon ca. CaCo-2 | 0.1 |
| Colon cancer tissue | 7.9 |
| Colon ca. SDW1116 | 0.1 |
| Colon ca. Colo-205 | 0.0 |
| Colon ca. SW-48 | 0.0 |
| Colon Pool | 39.0 |
| Small Intestine Pool | 20.7 |
| Stomach Pool | 20.9 |
| Bone Marrow Pool | 4.4 |
| Fetal Heart | 0.8 |
| Heart Pool | 6.2 |
| Lymph Node Pool | 15.7 |
| Fetal Skeletal Muscle | 3.6 |
| Skeletal Muscle Pool | 3.7 |
| Spleen Pool | 6.4 |
| Thymus Pool | 15.0 |
| CNS cancer (glio/astro) U87-MG | 26.2 |
| CNS cancer (glio/astro) U-118-MG | 12.2 |
| CNS cancer (neuro;met) SK-N-AS | 0.6 |
| CNS cancer (astro) SF-539 | 3.0 |
| CNS cancer (astro) SNB-75 | 57.4 |
| CNS cancer (glio) SNB-19 | 0.0 |
| CNS cancer (glio) SF-295 | 8.1 |
| Brain (Amygdala) Pool | 53.6 |
| Brain (cerebellum) | 31.0 |
| Brain (fetal) | 1.2 |
| Brain (Hippocampus) Pool | 51.4 |
| Cerebral Cortex Pool | 51.8 |
| Brain (Substantia nigra) Pool | 47.0 |
| Brain (Thalamus) Pool | 87.7 |
| Brain (whole) | 44.4 |
| Spinal Cord Pool | 100.0 |
| Adrenal Gland | 15.7 |
| Pituitary gland Pool | 5.4 |
| Salivary Gland | 0.7 |
| Thyroid (female) | 2.4 |
| Pancreatic ca. CAPAN2 | 0.0 |
| Pancreas Pool | 28.1 |

TABLE AAD
Panel 2.2

| | Rel. Exp.(%) |
|---|---|
| | Ag3857, Run |
| Tissue Name | 173762112 |
| Normal Colon | 5.8 |
| Colon cancer (OD06064) | 7.9 |
| Colon Margin (OD06064) | 12.2 |
| Colon cancer (OD06159) | 0.7 |
| Colon Margin (OD06159) | 6.3 |
| Colon cancer (OD06297-04) | 0.9 |
| Colon Margin (OD06297-05) | 7.2 |
| CC Gr.2 ascend colon (ODO3921) | 3.6 |
| CC Margin (ODO3921) | 2.4 |
| Colon cancer metastasis | 2.7 |
| (OD06104) | |
| Lung Margin (OD06104) | 9.9 |
| Colon mets to lung (OD04451-01) | 0.8 |
| Lung Margin (OD04451-02) | 7.9 |
| Normal Prostate | 3.1 |
| Prostate Cancer (OD04410) | 0.8 |
| Prostate Margin (OD04410) | 3.8 |
| Normal Ovary | 3.5 |
| Ovarian cancer (OD06283-03) | 0.3 |
| Ovarian Margin (OD06283-07) | 16.3 |
| Ovarian Cancer 064008 | 1.8 |
| Ovarian cancer (OD06145) | 11.7 |
| Ovarian Margin (OD06145) | 12.8 |
| Ovarian cancer (OD06455-03) | 0.1 |
| Ovarian Margin (OD06455-07) | 6.7 |
| Normal Lung | 7.1 |
| Invasive poor diff. lung adeno | 3.2 |
| (ODO4945-01 | |
| Lung Margin (ODO4945-03) | 5.7 |
| Lung Malignant Cancer | 6.8 |
| (OD03126) | |
| Lung Margin (OD03126) | 3.5 |
| Lung Cancer (OD05014A) | 6.4 |
| Lung Margin (OD05014B) | 33.9 |
| Lung cancer (OD06081) | 0.7 |
| Lung Margin (OD06081) | 2.9 |
| Lung Cancer (OD04237-01) | 2.8 |
| Lung Margin (OD04237-02) | 29.1 |
| Ocular Melanoma Metastasis | 34.2 |
| Ocular Melanoma Margin (Liver) | 10.2 |
| Melanoma Metastasis | 12.7 |
| Melanoma Margin (Lung) | 13.6 |
| Normal Kidney | 3.7 |
| Kidney Ca, Nuclear grade 2 | 16.6 |
| (OD04338) | |
| Kidney Margin (OD04338) | 6.7 |
| Kidney Ca Nuclear grade 1/2 | 24.0 |
| (OD04339) | |
| Kidney Margin (OD04339) | 14.6 |
| Kidney Ca, Clear cell type | 100.0 |
| (OD04340) | |
| Kidney Margin (OD04340) | 8.1 |
| Kidney Ca, Nuclear grade 3 | 13.0 |
| (OD04348) | |
| Kidney Margin (OD04348) | 47.6 |
| Kidney malignant cancer | 1.9 |
| (OD06204B) | |
| Kidney normal adjacent tissue | 12.4 |
| (OD06204E) | |
| Kidney Cancer (OD04450-01) | 5.2 |
| Kidney Margin (OD04450-03) | 8.0 |
| Kidney Cancer 8120613 | 0.6 |
| Kidney Margin 8120614 | 3.4 |
| Kidney Cancer 9010320 | 2.6 |
| Kidney Margin 9010321 | 4.1 |
| Kidney Cancer 8120607 | 7.2 |
| Kidney Margin 8120608 | 6.5 |
| Normal Uterus | 23.8 |
| Uterine Cancer 064011 | 8.5 |
| Normal Thyroid | 0.4 |
| Thyroid Cancer 064010 | 1.6 |
| Thyroid Cancer A302152 | 13.8 |
| Thyroid Margin A302153 | 0.8 |
| Normal Breast | 18.8 |
| Breast Cancer (OD04566) | 1.1 |
| Breast Cancer 1024 | 1.3 |
| Breast Cancer (OD04590-01) | 0.8 |
| Breast Cancer Mets | 7.4 |
| (OD04590-03) | |
| Breast Cancer Metastasis | 7.4 |
| (0D04655-05) | |
| Breast Cancer 064006 | 2.3 |
| Breast Cancer 9100266 | 1.5 |
| Breast Margin 9100265 | 6.9 |
| Breast Cancer A209073 | 1.8 |
| Breast Margin A2090734 | 6.3 |
| Breast cancer (OD06083) | 6.1 |
| Breast cancer node metastasis | 6.3 |
| (OD06083) | |
| Normal Liver | 1.6 |
| Liver Cancer 1026 | 5.5 |
| Liver Cancer 1025 | 2.1 |
| Liver Cancer 6004-T | 3.7 |
| Liver Tissue 6004-N | 3.6 |
| Liver Cancer 6005-T | 10.9 |
| Liver Tissue 6005-N | 4.1 |
| Liver Cancer 064003 | 5.7 |
| Normal Bladder | 3.2 |
| Bladder Cancer 1023 | 2.2 |
| Bladder Cancer A302173 | 15.2 |
| Normal Stomach | 14.1 |
| Gastric Cancer 9060397 | 0.8 |
| Stomach Margin 9060396 | 3.1 |
| Gastric Cancer 9060395 | 2.5 |
| Stomach Margin 9060394 | 8.2 |
| Gastric Cancer 064005 | 3.8 |

TABLE AAE
Panel 4.1D

| | Rel. Ex.(%) |
|---|---|
| | Ag3857, Run |
| Tissue Name | 170120945 |
| Secondary Th1 act | 1.9 |
| Secondary Th2 act | 0.4 |
| Secondary Tr1 act | 0.2 |
| Secondary Th1 rest | 1.0 |
| Secondary Th2 rest | 0.1 |
| Secondary Tr1 rest | 0.2 |
| Primary Th1 act | 0.9 |
| Primary Th2 act | 0.1 |
| Primary Tr1 act | 0.3 |
| Primary Th1 rest | 0.3 |
| Primary Th2 rest | 0.1 |
| Primary Tr1 rest | 0.1 |
| CD45RA CD4 lymphocyte act | 9.3 |
| CD45RO CD4 lymphocyte act | 0.4 |
| CD8 lymphocyte act | 0.1 |
| Secondary CD8 lymphocyte rest | 0.4 |
| Secondary CD8 lymphocyte act | 0.0 |
| CD4 lymphocyte none | 0.1 |
| 2ry Th1/Th2/Tr1_anti-CD95 | 0.4 |
| CH11 | |
| LAK cells rest | 6.0 |
| LAK cells IL-2 | 0.2 |
| LAK cells IL-2 + IL-12 | 0.2 |
| LAK cells IL-2 + IFN gamma | 0.4 |
| LAK cells IL-2 + IL-18 | 0.4 |
| LAK cells PMA/ionomycin | 4.1 |
| NK Cells IL-2 rest | 0.0 |
| Two Way MLR 3 day | 2.1 |
| Two Way MLR 5 day | 0.5 |
| Two Way MLR 7 day | 0.4 |
| PBMC rest | 0.1 |
| PBMC PWM | 0.4 |
| PBMC PHA-L | 0.3 |
| Ramos (B cell) none | 0.0 |
| Ramos (B cell) ionomycin | 0.0 |
| B lymphocytes PWM | 0.2 |
| B lymphocytes CD40L and IL-4 | 0.0 |
| EOL-1 dbcAMP | 3.2 |
| EOL-1 dbcAMP | 0.7 |
| PMA/ionomycin | |
| Dendritic cells none | 19.6 |
| Dendritic cells LPS | 9.3 |
| Dendritic cells anti-CD40 | 13.5 |
| Monocytes rest | 0.1 |
| Monocytes LPS | 8.9 |
| Macrophages rest | 0.0 |
| Macrophages LPS | 0.6 |
| HUVEC none | 0.0 |
| HUVEC starved | 0.0 |
| HUVEC IL-1beta | 0.0 |
| HUVEC IFN gamma | 0.1 |
| HUVEC TNF alpha + IFN gamma | 0.0 |
| HUVEC TNF alpha + IL4 | 0.0 |
| HUVEC IL-11 | 0.1 |
| Lung Microvascular EC none | 0.0 |
| Lung Microvascular EC TNFalpha + | 0.0 |
| IL-1beta | |
| Microvascular Dermal EC none | 0.0 |
| Microsvasular Dermal EC | 0.0 |
| TNFalpha + IL-1beta | |
| Bronchial epithelium TNFalpha + | 0.0 |
| IL1beta | |
| Small airway epithelium none | 0.1 |
| Small airway epithelium TNFalpha + | 0.0 |
| IL-1beta | |
| Coronery artery SMC rest | 0.0 |
| Coronery artery SMG TNFalpha + | 0.0 |
| IL-1beta | |
| Astrocytes rest | 0.6 |
| Astrocytes TNFalpha + IL-1beta | 1.6 |
| KU-812 (Basophil) rest | 0.0 |
| KU-812 (Basophil) | 0.1 |
| PMA/ionomycin | |
| CCD1106 (Keratinocytes) none | 0.0 |
| CCD1106 (Keratinocytes) | 0.0 |
| TNFalpha + IL-1beta | |
| Liver cirrhosis | 2.2 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1beta | 0.0 |
| Lung fibroblast none | 1.9 |
| Lung fibroblast TNF alpha + | 1.2 |
| IL-1beta | |
| Lung fibroblast IL-4 | 1.3 |
| Lung fibroblast IL-9 | 1.8 |
| Lung fibroblast IL-13 | 0.9 |
| Lung fibroblast IFN gamma | 1.0 |
| Dermal fibroblast CCD1070 rest | 26.1 |
| Dermal fibroblast CCD1070 TNF | 17.7 |
| alpha | |
| Dermal fibroblast CCD1070 | 18.0 |
| IL-1beta | |
| Dermal fibroblast IFN gamma | 62.4 |
| Dermal fibroblast IL-4 | 100.0 |
| Dermal Fibroblasts rest | 49.0 |
| Neutrophils TNFa + LPS | 0.1 |
| Neutrophils rest | 0.1 |
| Colon | 2.7 |
| Lung | 50.7 |
| Thymus | 3.2 |
| Kidney | 13.0 |

TABLE AAF
Panel 5 Islet

| | Rel. Exp.(%) |
|---|---|
| | Ag3857, Run |
| Tissue Name | 172213998 |
| 97457_Patient-02go_adipose | 59.5 |
| 97476_Patient-07sk_skeletal muscle | 18.0 |
| 97477_Patient-07ut_uterus | 25.5 |
| 97478_Patient-07pl_placenta | 52.1 |
| 99167_Bayer Patient 1 | 53.2 |
| 97482_Patient-08ut_uterus | 21.5 |
| 97483_Patient-08pl_placenta | 34.4 |
| 97486_Patient-09sk_skeletal muscle | 1.9 |
| 97487_Patient-09ut_uterus | 22 2 |
| 97488_Patient-09pl_placenta | 27.9 |
| 97492_Patient-10ut_uterus | 41.2 |
| 97493_Patient-10pl_placenta | 75.8 |
| 97495_Patient-11go_adipose | 13.8 |
| 97496_Patient-11sk_skeletal muscle | 2.8 |
| 97497_Patient-11ut_uterus | 36.9 |
| 97498_Patient-11pl_placenta | 13.4 |
| 97500_Patient-12go_adipose | 42.0 |
| 97501_Patient-12sk_skeletal muscle | 8.8 |
| 97502_Patient-12ut_uterus | 38.2 |
| 97503_Patient-12pl_placenta | 22.7 |
| 94721_Donor 2 U - | 22.5 |
| A_Mesenchymal Stem Cells | |
| 94722_Donor 2 U - | 12.9 |
| A_Mesenchymal Stem Cells | |
| 94723_Donor 2 U - | 25.5 |
| C_Mesenchymal Stem Cells | |
| 94709_Donor 2 AM - A_adipose | 46.0 |
| 94710_Donor 2 AM - B_adipose | 22.1 |
| 94711_Donor 2 AM - C_adipose | 17.6 |
| 94712_Donor 2 AD - A_adipose | 78.5 |
| 94713_Donor 2 AD - B_adipose | 80.1 |
| 94714_Donor 2 AD - C_adipose | 100.0 |
| 94742_Donor 3 U - A_Mesenchymal | 12.9 |
| Stem Cells | |
| 94743_Donor 3 U - B_Mesenchymal | 30.1 |
| Stem Cells | |
| 94730_Donor 3 AM - A_adipose | 50.7 |
| 94731_Donor 3 AM - B_adipose | 25.2 |
| 94732_Donor 3 AM - C_adipose | 23.5 |
| 94733_Donor 3 AD - A_adipose | 90.8 |
| 94734_Donor 3 AD - B_adipose | 27.2 |
| 94735_Donor 3 AD - C_adipose | 77.9 |
| 77138_Liver_HepG2untreated | 3.9 |
| 73556_Heart_Cardiac stromal cells | 0.1 |
| (primary) | |
| 81735_Small Intestine | 11.4 |
| 72409_Kidney_Proximal Convoluted | 1.4 |
| 82685_Small intestine_Duodenum | 4.1 |
| 90650_Adrenal_Adrenocortical | 5.1 |
| adenoma | |
| 72410_Kidney_HRCE | 1.7 |
| 72411_Kidney_HRE | 0.2 |
| 73139_Uterus_Uterine smooth | 2.5 |
| muscle cells | |

TABLE AAG
Panel 5D

| | Rel. Exp.(%) |
|---|---|
| | Ag3857, Run |
| Tissue Name | 170222682 |
| 97457_Patient-02go_adipose | 54.7 |
| 97476_Patient-07sk_skeletal muscle | 16.7 |
| 97477_Patient-07ut_uterus | 21.6 |
| 97478_Patient-07pl_placenta | 52.1 |
| 97481_Patient-08sk_skeletal muscle | 22.5 |
| 97482_Patient-08ut_uterus | 21.8 |
| 97483_Patient-08pl_placenta | 39.5 |
| 97486_Patient-09sk_skeletal muscle | 2.9 |
| 97487_Patient-09ut_uterus | 14.6 |
| 97488_Patient-09pl_placenta | 29.3 |
| 97492_Patient-10ut_uterus | 31.2 |
| 97493_Patient-10pl_placenta | 62.4 |
| 97495_Patient-11go_adipose | 16.3 |
| 97496_Patient-11sk_skeletal muscle | 1.9 |
| 97497_Patient-11ut_uterus | 33.7 |
| 97498_Patient-11pl_placenta | 20.2 |
| 97500_Patient-12go_adipose | 32.1 |
| 97501_Patient-12sk_skeletal muscle | 6.7 |
| 97502_Patient-12ut_uterus | 35.6 |
| 97503_Patient-12pl_placenta | 25.0 |
| 94721_Donor 2 U - | 27.2 |
| A_Mesenchymal Stem Cells | |
| 94722_Donor 2 U - | 15.7 |
| B_Mesenchymal Stem Cells | |
| 94723_Donor 2 U - | 18.3 |
| C_Mesenchymal Stem Cells | |
| 94709_Donor 2 AM - A_adipose | 39.8 |
| 94710_Donor 2 AM - B_adipose | 29.9 |
| 94711_Donor 2 AM - C_adipose | 19.9 |
| 94712_Donor 2 AD - A_adipose | 76.3 |
| 94713_Donor 2 AD - B_adipose | 100.0 |
| 94714_Donor 2 AD - C_adipose | 93.3 |
| 94742_Donor 3 U - A_Mesenchymal | 18.7 |
| Stem Cells | |
| 94743_Donor 3 U - B_Mesenchymal | 27.9 |
| Stem Cells | |
| 94730_Donor 3 AM - A_adipose | 52.9 |
| 94731_Donor 3 AM - B_adipose | 28.1 |
| 94732_Donor 3 AM - C_adipose | 32.1 |
| 94733_Donor 3 AD - A_adipose | 77.9 |
| 94734_Donor 3 AD - B_adipose | 41.2 |
| 94735_Donor 3 AD - C_adipose | 67.8 |
| 77138_Liver_HepG2untreated | 4.6 |
| 73556_Heart_Cardiac stromal cells | 0.2 |
| (primary) | |
| 81735_Small Intestine | 9.4 |
| 72409_Kidney_Proximal Convoluted | 2.2 |
| Tubule | |
| 82685_Small intestine_Duodenum | 25.0 |
| 90650_Adrenal_Adrenocortical | 3.8 |
| adenoma | |
| 72410_Kidney_HRCE | 2.2 |
| 72411_Kidney_HRE | 0.3 |
| 73139_Uterus_Uterine smooth | 2.6 |
| muscle cells | |

CNS_neurodegeneration_v1.0 Summary: Ag3857 This panel does not show differential expression of this gene in Alzheimer's disease. However, this expression profile confirms the presence of this gene in the brain. Please see Panel 1.5 for discussion of this gene in the central nervous system.

General_screening_panel_v1.5 Summary: Ag3857 Highest expression of the CG93541-01 gene is seen in spinal cord (CT=25.1). This gene is also expressed at high regions throughout the CNS. Thus, expression of this gene may be used to differentiate between brain derived samples and other samples on this panel and as a marker of brain tissue. This gene is homologous to autotaxin, a gene that is enriched in the spinal cord and brain of rats and may be involved in oligodendrocyte function (Fuss B. J Neurosci Dec. 1, 1997;17(23):9095-103). Therefore, the strong association of this gene with the CNS and its homology to autotaxin suggest that therapeutic modulation of this gene or gene product may be useful in the treatment of neurologic disease and specifically demyelinating diseases such as multiple sclerosis.

In addition, this gene is expressed at much higher levels in fetal lung and liver tissue (CTs=26.5-27.5) when compared to expression in the adult counterpart (CTs=31-33). Thus, expression of this gene may be used to differentiate between the fetal and adult sources of these tissues. The relative overexpression of this gene in these fetal tissues also suggests that the protein product may enhance growth or development of these organs in the fetus and thus may also act in a regenerative capacity in the adult. Therefore, therapeutic modulation of the protein encoded by this gene may be useful in the treatment of diseases that affect these organs.

Among metabolic tissues, this gene is highly expressed in pancreas, adrenal, fetal liver, and adipose. It is expressed at moderate levels in pituitary, thyroid, heart and fetal and adult skeletal muscle, with low but significant expression in liver and fetal heart. Please see panel 5I for further discussion of this gene.

Panel 2.2 Summary: Ag3857 Highest expression is seen in kidney cancer (CT=28.2). In addition, this gene is more highly expressed in kidney cancer than in the corresponding normal adjacent tissue. Thus, expression of this gene could be used as a marker of this cancer. Furthemore, therapeutic modulation of the expression or function of this gene product may be useful in the treatment of kidney cancer.

Panel 4.1D Summary: Ag3857 Highest expression of this gene is seen in dermal fibroblasts treated with IL-4 (CT=25.3). In addition, high levels of expression are seen in a cluster of samples derived from dermal fibroblasts. Thus, expression of this gene may be used as a marker of this cell. In addition, therapeutic modulation of the expression or function of this gene may be useful in the treatment of skin disorders, including psoriasis.

Panel 5 Islet Summary: Ag3857 Highest expression of this gene is detected in differentiated adipose (CT=29.2). This gene shows widespread expression in this panel, with signifacant expression in human islets (CT=30). This gene codes for Autotaxin-t (ATX). ATX is a bifunctional enzyme with phosphodiesterase I and nucleotide pyrophosphatase activities. ATX is expressed in pancreatic islets and at CuraGen using GeneCalling studies it was found that the rat orthologue (PDE1) is down-regulated in good insulin-secreting versus poor-secreting cell lines. Therefore, inhibition of ATX would lead to elevation of extracellular ATP resulting in activation of purinergic receptors, thus increasing insulin secretion. Therefore, an antagonist of ATX can improve insulin secretion in Type 2 diabetes.

Kawagoe, H.; Soma, O.; Goji, J.; Nishimura, N.; Narita, M.; Inazawa, J.; Nakamura, H.; Sano, K. Molecular cloning and chromosomal assignment of the human brain-type phosphodiesterase I/nucleotide pyrophosphatase gene (PDNP2). Genomics 30: 380-384, 1995. PubMed ID: 8586446; Murata, J.; Lee, H. Y.; Clair, T.; Krutzsch, H. C.; Arestad, A. A.; Sobel, M. E.; Liotta, L. A.; Stracke, M. L. cDNA cloning of human tumor motility-stimulating protein, autotaxin, reveals a homology with phosphodiesterases. J. Biol. Chem. 269: 30479-30484, 1994. PubMed ID: 7982964; Narita, M.; Goji, J.; Nakamura, H.; Sano, K. Molecular cloning, expression, and localization of a brain-specific phosphodiesterase I/nucleotide (PD-I-alpha) from rat brain. J. Biol. Chem. 269: 28235-28242, 1994. PubMed ID: 7961762; Piao, J.-H.; Matsuda, Y.; Nakamura, H.; Sano, K. Assignment of Pdnp2, the gene encoding phosphodiesterase I/nucleotide pyrophosphatase 2, to mouse chromosome 15D2. Cytogenet. Cell Genet. 87: 172-174, 1999. PubMed ID: 10702660

Panel 5D Summary: Ag 3857 Highest expression of this gene is seen in adipose (CT=28.7). Moderate levels of expression are seen in other metabolic tissues on this panel, including skeletal muscle. Overall, these results are in agreement with the results in Panel 5I. Please see that panel for further discussion of the role of this gene in metabolic disease.

AC. CG93735-01: Human Adenylate Kinase 3 Alpha-Like Protein.

Expression of gene CG93735-01 was assessed using the primer-probe set Ag3926, described in Table ACA. Results of the RTQ-PCR runs are shown in Tables ACB, ACC and ACD.

TABLE ACA

Probe Name Ag3926

| | | | |
|---|---|---|---|
| | | | SEQ ID |
| Primers | Length | Start Position | No |

| | | | | |
|---|---|---|---|---|
| Forward | 5′-gtatagctggctgttggatg-3′ | 20 | 392 | 473 |
| Probe | TET-5′-ttttccaaggacacttccacaggcagaa-3′-TAMRA | 29 | 0 | 474 |
| Reverse | 5′-cgatctgataagctctatctag-3′ | 22 | 444 | 475 |

TABLE ACB
General screening panel v1.5

| | Rel. Exp.(%) |
|---|---|
| | Ag3926, Run |
| Tissue Name | 244371054 |
| Adipose | 15.5 |
| Melanoma* Hs688(A).T | 25.3 |
| Melanoma* Hs688(B).T | 23.8 |
| Melanoma* M14 | 18.9 |
| Melanoma* LOXIMVI | 15.1 |
| Melanoma* SK-MEL-5 | 19.9 |
| Squamous cell carcinoma SCC-4 | 11.2 |
| Testis Pool | 7.5 |
| Prostate ca.* (bone met) PC-3 | 18.6 |
| Prostate Pool | 16.7 |
| Placenta | 6.9 |
| Uterus Pool | 8.5 |
| Ovarian ca. OVCAR-3 | 36.9 |
| Ovarian ca. SK-OV-3 | 13.8 |
| Ovarian ca. OVCAR-4 | 2.6 |
| Ovarian ca. OVCAR-5 | 43.8 |
| Ovarian ca. IGROV-1 | 16.6 |
| Ovarian ca. OVCAR-8 | 11.4 |
| Ovary | 20.0 |
| Breast ca. MCF-7 | 24.1 |
| Breast ca. MDA-MB-231 | 17.0 |
| Breast ca. BT 549 | 42.3 |
| Breast ca. T47D | 6.5 |
| Breast ca. MDA-N | 11.3 |
| Breast Pool | 21.2 |
| Trachea | 12.6 |
| Lung | 7.1 |
| Fetal Lung | 31.6 |
| Lung ca. NCI-N417 | 6.9 |
| Lung ca. LX-1 | 23.2 |
| Lung ca. NCI-H146 | 7.3 |
| Lung ca. SHP-77 | 5.7 |
| Lung ca. A549 | 27.2 |
| Lung ca. NCI-H526 | 10.7 |
| Lung ca. NCI-H23 | 14.6 |
| Lung ca. NCI-H460 | 11.6 |
| Lung ca. HOP-62 | 32.5 |
| Lung ca. NCI-H522 | 23.7 |
| Liver | 8.7 |
| Fetal Liver | 40.6 |
| Liver ca. HepG2 | 31.4 |
| Kidney Pool | 27.2 |
| Fetal Kidney | 35.4 |
| Renal ca. 786-0 | 26.4 |
| Renal ca. A498 | 9.3 |
| Renal ca. ACHN | 18.3 |
| Renal ca. UO-31 | 25.7 |
| Renal ca. TK-10 | 27.2 |
| Bladder | 20.6 |
| Gastric ca. (liver met.) NCI-N87 | 37.9 |
| Gastric ca. KATO III | 66.4 |
| Colon ca. SW-948 | 12.5 |
| Colon ca. SW480 | 53.6 |
| Colon ca.* (SW480 met) SW620 | 22.5 |
| Colon ca. HT29 | 16.5 |
| Colon ca. HCT-116 | 21.2 |
| Colon ca. CaCo-2 | 27.0 |
| Colon cancer tissue | 24.3 |
| Colon ca. SW1116 | 4.8 |
| Colon ca. Colo-205 | 5.1 |
| Colon ca. SW-48 | 10.9 |
| Colon Pool | 22.5 |
| Small Intestine Pool | 19.2 |
| Stomach Pool | 11.3 |
| Bone Marrow Pool | 11.7 |
| Fetal Heart | 13.7 |
| Heart Pool | 21.6 |
| Lymph Node Pool | 25.3 |
| Fetal Skeletal Muscle | 7.7 |
| Skeletal Muscle Pool | 100.0 |
| Spleen Pool | 11.0 |
| Thymus Pool | 19.8 |
| CNS cancer (glio/astro) U87-MG | 24.1 |
| CNS cancer (glio/astro) U-118-MG | 27.5 |
| CNS cancer (neuro;met) SK-N-AS | 13.3 |
| CNS cancer (astro) SF-539 | 10.7 |
| CNS cancer (astro) SNB-75 | 43.5 |
| CNS cancer (glio) SNB-19 | 14.2 |
| CNS cancer (glio) SF-295 | 54.0 |
| Brain (Amygdala) Pool | 8.3 |
| Brain (cerebellum) | 19.9 |
| Brain (fetal) | 9.9 |
| Brain (Hippocampus) Pool | 12.2 |
| Cerebral Cortex Pool | 11.0 |
| Brain (Substantia nigra) Pool | 11.7 |
| Brain (Thalamus) Pool | 16.4 |
| Brain (whole) | 8.2 |
| Spinal Cord Pool | 12.9 |
| Adrenal Gland | 26.1 |
| Pituitary gland Pool | 2.9 |
| Salivary Gland | 6.7 |
| Thyroid (female) | 5.9 |
| Pancreatic ca. CAPAN2 | 17.4 |
| Pancreas Pool | 18.2 |

TABLE ACC
General screening panel v1.6

| | Rel. Exp.(%) |
|---|---|
| | Ag3926, Run |
| Tissue Name | 277230942 |
| Adipose | 24.1 |
| Melanoma* Hs688(A).T | 42.3 |
| Melanoma* Hs688(B).T | 31.2 |
| Melanoma* M14 | 41.2 |
| Melanoma* LOXIMVI | 0.0 |
| Melanoma* SK-MEL-5 | 41.2 |
| Squamous cell carcinoma SCC-4 | 14.4 |
| Testis Pool | 14.3 |
| Prostate ca.* (bone met) PC-3 | 26.6 |
| Prostate Pool | 23.0 |
| Placenta | 13.1 |
| Uterus Pool | 8.5 |
| Ovarian ca. OVCAR-3 | 0.0 |
| Ovarian ca. SK-OV-3 | 26.1 |
| Ovarian ca. OVCAR-4 | 25.9 |
| Ovarian ca. OVCAR-5 | 84.1 |
| Ovarian ca. IGROV-1 | 26.2 |
| Ovarian ca. OVCAR-8 | 22.1 |
| Ovary | 25.7 |
| Breast ca. MCF-7 | 30.6 |
| Breast ca. MDA-MB-231 | 24.7 |
| Breast ca. BT 549 | 72.2 |
| Breast ca. T47D | 12.6 |
| Breast ca. MDA-N | 14.8 |
| Breast Pool | 36.1 |
| Trachea | 18.6 |
| Lung | 11.0 |
| Fetal Lung | 43.8 |
| Lung ca. NCI-N417 | 12.8 |
| Lung ca. LX-1 | 31.4 |
| Lung ca. NCI-H146 | 12.2 |
| Lung ca. SHP-77 | 10.7 |
| Lung ca. A549 | 50.3 |
| Lung ca. NCI-H526 | 21.0 |
| Lung ca. NCI-H23 | 17.4 |
| Lung ca. NCI-H460 | 14.9 |
| Lung ca. HOP-62 | 48.6 |
| Lung ca. NCI-H522 | 30.6 |
| Liver | 0.0 |
| Fetal Liver | 57.4 |
| Liver ca. HepG2 | 39.8 |
| Kidney Pool | 45.7 |
| Fetal Kidney | 46.3 |
| Renal ca. 786-0 | 25.0 |
| Renal ca. A498 | 20.6 |
| Renal ca. ACHN | 27.5 |
| Renal ca. UO-31 | 43.5 |
| Renal ca. TK-10 | 60.7 |
| Bladder | 39.2 |
| Gastric ca. (liver met.) NCI-N87 | 76.8 |
| Gastric ca. KATO III | 95.9 |
| Colon ca. SW-948 | 20.3 |
| Colon ca. SW480 | 7.8 |
| Colon ca.* (SW480 met) SW620 | 30.6 |
| Colon ca. HT29 | 26.6 |
| Colon ca. HCT-116 | 46.0 |
| Colon ca. CaCo-2 | 47.6 |
| Colon cancer tissue | 34.6 |
| Colon ca. SW1116 | 7.4 |
| Colon ca. Colo-205 | 10.1 |
| Colon ca. SW-48 | 14.3 |
| Colon Pool | 35.8 |
| Small Intestine Pool | 25.0 |
| Stomach Pool | 18.0 |
| Bone Marrow Pool | 21.0 |
| Fetal Heart | 26.6 |
| Heart Pool | 29.5 |
| Lymph Node Pool | 46.0 |
| Fetal Skeletal Muscle | 13.7 |
| Skeletal Muscle Pool | 28.5 |
| Spleen Pool | 14.9 |
| Thymus Pool | 26.4 |
| CNS cancer (glio/astro) U87-MG | 51.1 |
| CNS cancer (glio/astro) U-118-MG | 40.3 |
| CNs cancer (neuro;met) SK-N-AS | 22.5 |
| CNS cancer (astro) SF-539 | 18.0 |
| CNS cancer (astro) SNB-75 | 56.3 |
| CNS cancer (glio) SNB-19 | 24.7 |
| CNS cancer (glio) SF-295 | 100.0 |
| Brain (Amygdala) Pool | 12.6 |
| Brain (cerebellum) | 34.4 |
| Brain (fetal) | 15.1 |
| Brain (Hippocampus) Pool | 20.0 |
| Cerebral Cortex Pool | 18.9 |
| Brain (Substantia nigra) Pool | 13.2 |
| Brain (Thalamus) Pool | 22.1 |
| Brain (whole) | 11.8 |
| Spinal Cord Pool | 20.9 |
| Adrenal Gland | 50.3 |
| Pituitary gland Pool | 5.4 |
| Salivary Gland | 8.2 |
| Thyroid (female) | 10.2 |
| Pancreatic ca. CAPAN2 | 27.0 |
| Pancreas Pool | 23.5 |

TABLE ACD
Panel 5 Islet

| | Rel. Exp.(%) |
|---|---|
| | Ag3926, Run |
| Tissue Name | 227742519 |
| 97457_Patient-02go_adipose | 29.7 |
| 97476_Patient-07sk_skeletal muscle | 22.1 |
| 97477_Patient-07ut_uterus | 16.3 |
| 97478_Patient-07pl_placenta | 29.3 |
| 99167_Bayer Patient 1 | 18.4 |
| 97482_Patient-08ut_uterus | 12.5 |
| 97483_Patient-08pl_placenta | 38.2 |
| 97486_Patient-09sk_skeletal muscle | 16.5 |
| 97487_Patient-09ut_uterus | 36.1 |
| 97488_Patient-09pl_placenta | 13.0 |
| 97492_Patient-10ut_uterus | 25.0 |
| 97493_Patient-10pl_placenta | 39.5 |
| 97495_Patient-11go_adipose | 28.5 |
| 97496_Patient-11sk_skeletal muscle | |
| 97497_Patient-11ut_uterus | 31.4 |
| 97498_Patient-11pl_placenta | 17.7 |
| 97500_Patient-12go_adipose | 24.1 |
| 97501_Patient-12sk_skeletal muscle | 78.5 |
| 97502_Patient-12ut_uterus | 26.8 |
| 97503_Patient-12pl_placenta | 18.7 |
| 94721_Donor 2 U - | 17.6 |
| A_Mesenchymal Stem Cells | |
| 94722_Donor 2 U - | 12.1 |
| B_Mesenchymal Stem Cells | |
| 94723_Donor 2 U - | 18.6 |
| C_Mesenchymal Stem Cells | |
| 94709_Donor 2 AM - A_adipose | 26.4 |
| 94710_Donor 2 AM - B_adipose | 17.4 |
| 94711_Donor 2 AM - C_adipose | 14.4 |
| 94712_Donor 2 AD - A_adipose | 40.1 |
| 94713_Donor 2 AD - B_adipose | 42.3 |
| 94714_Donor 2 AD - C_adipose | 36.3 |
| 94742_Donor 3 U - A_Mesenchymal | 4.7 |
| Stem Cells | |
| 94743_Donor 3 U - B_Mesenchymal | 12.0 |
| Stem Cells | |
| 94730_Donor 3 AM - A_adipose | 24.7 |
| 94731_Donor 3 AM - B_adipose | 10.8 |
| 94732_Donor 3 AM - C_adipose | 14.2 |
| 94733_Donor 3 AD - A_adipose | 36.1 |
| 94734_Donor 3 AD - B_adipose | 13.0 |
| 94735_Donor 3 AD - C_adipose | 33.2 |
| 77138_Liver_HepG2untreated | 100.0 |
| 73556_Heart_Cardiac stromal cells | 15.7 |
| (primary) | |
| 81735_Small Intestine | 41.5 |
| 72409_Kidney_Proximal Convoluted | 21.3 |
| Tubule | |
| 82685_Small intestine_Duodenum | 29.1 |
| 90650_Adrenal_Adrenocortical | 15.3 |
| adenoma | |
| 72410_Kidney_HRCE | 40.6 |
| 72411_Kidney_HRE | 39.8 |
| 73139_Uterus_Uterine smooth | 19.3 |
| muscle cells | |

General_screening_panel_v1.5 Summary: Ag3926 Highest expression of this gene is detected in skeletal muscle (CT=24.1). High expression of this gene is also seen tissues with metabolic/endocrine function including pancreas, adipose, adrenal gland, thyroid, pituitary gland, skeletal muscle, heart, liver and the gastrointestinal tract. This gene codes for adenylate kinase 3 alpha (AK3 alpha). In the GeneCalling studies at Curagen AK3 alpha was found to be up-regulated in adipose of diabetic GK rats. The over-expression of the phosphotransferase AK3 alpha in the adipocytes of the diabetic GK rat suggests a shift in mitochondrial energy production, and is suggestive for lower levels of cAMP in the diabetic state. cAMP levels have an impact on the insulin responsiveness of tissues, since it activates one of the important mediators of the insulin signaling pathway, AMP kinase. Therefore, inhibition of AK3 alpha may be an effective way to enhance insulin sensitivity in the metabolic tissues and may be used for therapy against diabetes. In addition, AMP kinase can also phosphorylate and inactivate acetyl-CoA carboxylase (ACC), which results in a decrease in malonyl-CoA production and, as a consequence, causes an increase in fatty acid oxidation in adipose tissue. Knock-outs of ACC2, for example, have decreased body weight even though they have increased food intake (Abu-Elheiga et al., Science 291: 2613-2626; 2001). Therefore, inhibitors of AK3 alpha may be effective therapeutics against obesity.

Moderate to high expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.6 Summary: Ag3926 Highest expression of this gene is detected in brain cancer SF-295 cell line (CT=25.3). This gene shows ubiquitous expression which correlates with expression seen in panel 1.5. Please see panel 1.5 for further discussion of this gene.

Panel 5 Islet Summary: Ag3926 Highest expression of this gene is detected in a liver cancer HepG2 cell line (CT=28.9). This panel confirms the findings of panel 1.5 that the target is highly expressed in metabolic tissues including muscle and adipose.

AD. CG93817-01: GPCR Olfacotry Receptor-Like Protein.

Expression of gene CG93817-01 was assessed using the primer-probe set Ag1653, described in Table ADA. Results of the RTQ-PCR runs are shown in Tables ADB and ADC.

TABLE ADA

Probe Name Ag1653

| | | | |
|---|---|---|---|
| | | | SEQ ID |
| Primers | Length | Start Position | No |

| | | | | |
|---|---|---|---|---|
| Forward | 5′-tctcctttctggacatctggta-3′ | 22 | 218 | 476 |
| Probe | TET-5′-tccaatgctggcaaactttgtttcag-3′-TAMRA | 26 | 258 | 477 |
| Reverse | 5′gcaccctgagaatgaaatagtg-3′ | 22 | 291 | 478 |

TABLE ADB
General screening panel v1.6

| | Rel. Exp.(%) |
|---|---|
| | Ag1653, Run |
| Tissue Name | 277227134 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 1.9 |
| Melanoma* SK-MEL-5 | 0.0 |
| Squamous cell carcinoma SCC-4 | 0.0 |
| Testis Pool | 5.9 |
| Prostate ca.* (bone met) PC-3 | 6.9 |
| Prostate Pool | 1.0 |
| Placenta | 0.0 |
| Uterus Pool | 0.0 |
| Ovarian ca. OVCAR-3 | 28.9 |
| Ovarian ca. SK-OV-3 | 1.5 |
| Ovarian ca. OVCAR-4 | 2.7 |
| Ovarian ca. OVCAR-5 | 5.3 |
| Ovarian ca. IGROV-1 | 3.5 |
| Ovarian ca. OVCAR-8 | 11.1 |
| Ovary | 20.7 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 1.0 |
| Lung | 0.0 |
| Fetal Lung | 1.5 |
| Lung ca. NCI-N417 | 2.2 |
| Lung ca. LX-1 | 0.0 |
| Lung ca. NCI-H146 | 1.7 |
| Lung ca. SHP-77 | 0.0 |
| Lung ca. A549 | 1.1 |
| Lung ca. NCI-H526 | 0.0 |
| Lung ca. NCI-H23 | 0.0 |
| Lung ca. NCI-H460 | 0.0 |
| Lung ca. HOP-62 | 9.5 |
| Lung ca. NCI-H522 | 0.0 |
| Liver | 0.0 |
| Fetal Liver | 1.2 |
| Liver ca. HepG2 | 40.1 |
| Kidney Pool | 0.9 |
| Fetal Kidney | 2.5 |
| Renal ca. 786-0 | 0.0 |
| Renal ca. A498 | 0.0 |
| Renal ca. ACHN | 0.0 |
| Renal ca. UO-31 | 43.8 |
| Renal ca. TK-10 | 44.8 |
| Bladder | 16.0 |
| Gastric ca. (liver met.) NCI-N87 | 0.0 |
| Gastric ca. KATO III | 3.2 |
| Colon ca. SW-948 | 0.0 |
| Colon ca. SW480 | 13.3 |
| Colon ca.* (SW480 met) SW620 | 35.6 |
| Colon ca. HT29 | 15.0 |
| Colon ca. HCT-1116 | 0.0 |
| Colon ca. CaCo-2 | 5.7 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 3.6 |
| Small Intestine Pool | 0.0 |
| Stomach Pool | 0.0 |
| Bone Marrow Pool | 1.8 |
| Fetal Heart | 9.3 |
| Heart Pool | 6.6 |
| Lymph Node Pool | 0.0 |
| Fetal Skeletal Muscle | 2.4 |
| Skeletal Muscle Pool | 0.0 |
| Spleen Pool | 5.3 |
| Thymus Pool | 11.4 |
| CNS cancer (glio/astro) U87-MG | 0.0 |
| CNS cancer (glio/astro) U-118-MG | 0.0 |
| CNS cancer (neuro;met) SK-N-AS | 2.4 |
| CNS cancer (astro) SF-539 | 1.9 |
| CNS cancer (astro) SNB-75 | 0.0 |
| CNS cancer (glio) SNB-19 | 0.0 |
| CNS cancer (glio) SF-295 | 11.3 |
| Brain (Amygdala) Pool | 4.4 |
| Brain (cerebellum) | 0.0 |
| Brain (fetal) | 5.3 |
| Brain (Hippocampus) Pool | 4.1 |
| Cerebral Cortex Pool | 4.7 |
| Brain (Substantia nigra) Pool | 4.5 |
| Brain (Thalamus) Pool | 4.6 |
| Brain (whole) | 1.5 |
| spinal Cord Pool | 7.5 |
| Adrenal Gland | 1.7 |
| Pituitary gland Pool | 11.3 |
| Salivary Gland | 0.0 |
| Thyroid (female) | 2.0 |
| Pancreatic ca. CAPAN2 | 100.0 |
| Pancreas Pool | 27.4 |

TABLE ADC
Panel 4D

| | Rel. | | Rel. |
|---|---|---|---|
| | Ep. (%) | | Exp. (%) |
| | Ag1653, | | Ag1653, |
| | Run | | Run |
| Tissue Name | 165762957 | Tissue Name | 165762957 |
| Secondary Th1 act | 0.0 | HUVEC IL-1beta | 0.0 |
| Secondary Th2 act | 10.8 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 5.7 |
| Primary Th1 act | 0.0 | Lung Microvascular EC TNF alpha + IL-1beta | 4.0 |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 3.6 | Bronchial epithelium TNF alpha + IL1beta | 0.0 |
| Primary Th2 rest | 0.0 | Small airway epithelium none | 3.7 |
| Primary Tr1 rest | 0.0 | Small airway epithelium TNF alpha + IL-1beta | 11.3 |
| CD45RA CD4 lymphocyte act | 0.0 | Coronery artery SMC rest | 0.0 |
| CD45RO CD4 lymphocyte act | 0.0 | Coronery artery SMC TNF alpha + IL-1beta | 0.0 |
| CD8 lymphocyte act | 0.0 | Astrocytes rest | 0.0 |
| Secondary CD8 lymphocyte rest | 0.0 | Astrocytes TNF alpha + IL-1beta | 6.3 |
| Secondary CD8 lymphocyte act | 0.0 | KU-812 (Basophil) rest | 3.0 |
| CD4 lymphocyte none | 0.0 | KU-812 (Basophil) | 4.2 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 5.2 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 8.3 |
| LAK cells IL-2 + IFN gamma | 11.6 | NCI-H292 none | 0.0 |
| LAK cells IL-2 + IL-18 | 3.8 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + IL-1beta | 0.0 |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 6.2 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1beta | 0.0 |
| PMA/ionomycin | | | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 17.4 |
| Monocytes rest | 0.0 | IBD Crohn's | 7.1 |
| Monocytes LPS | 2.8 | Colon | 6.9 |
| Macrophages rest | 0.0 | Lung | 0.0 |
| Macrophages LPS | 0.0 | Thymus | 4.2 |
| HUVEC none | 0.0 | Kidney | 0.0 |
| HUVEC starved | 0.0 | | |

General_screening_panel_v1.6 Summary: Ag1653 Highest expression of this gene is seen in a pancreatic cancer CAPAN2 cell line (CT=32.9). Low expression of this gene is also seen in a few cancer cell line derived from colon, renal, liver and ovarian cancers. Therefore, expression of this gene may be used as diagnostic marker to dectect the presence of these cancers and also therapeutic modulation of this gene or the GPCR encoded by this gene via antibodies or small molecule drug may be useful in the treatment of pancreatic, colon, renal, liver and ovarian cancers.

Panel 4D Summary: Ag1653 Expression of this gene is detected in IBD colitis 1 (CT=29.1) and in liver cirrhosis (CT=32.7). Therefore, antibodies that block the function of the putative GPCR encoded by this gene may be useful therapeutics in the treatment of colitis or cirrhosis.

AE. CG96859-03: HMG-COA Lyase Precursor-Like Protein.

Expression of gene CG96859-03 was assessed using the primer-probe set Ag4080, described in Table AEA.

TABLE AEA

Probe Name Ag4O8O

| | | | |
|---|---|---|---|
| | | | SEQ ID |
| Primers | Length | Start Position | No |

| | | | | |
|---|---|---|---|---|
| Forward | 5′-gccaaggaagtagtcatctttg-3′ | 22 | 646 | 479 |
| Probe | TET-5′-tgcctcagagctcttcaccaagaaga-3′-TAMRA | 26 | 616 | 480 |
| Reverse | 5′-gcgtcaaacctctgaaaactct-3′ | 22 | 573 | 481 |

### Example D: Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV1b SNP Data (CG101719-04)

Twenty-four polymorphic variants of NOV1b have been identified and are shown in Table 33A

TABLE 33A
Variants of NOV1b

| | Nucleotides | | | Amino Acids | | |
|---|---|---|---|---|---|---|
| Variant | Position | Initial | Modified | Position | Initial | Modified |
| 13379547 | 264 | T | C | 50 | Leu | Pro |
| 13375003 | 554 | A | G | 147 | Asn | Asp |
| 13374994 | 748 | C | T | 211 | Ala | Ala |
| 13374995 | 752 | T | C | 213 | Trp | Arg |
| 13374996 | 765 | T | C | 217 | Met | Thr |
| 13379549 | 788 | A | G | 225 | Lys | Glu |
| 13378470 | 877 | G | C | 254 | Arg | Arg |
| 13374549 | 999 | T | C | 295 | Val | Ala |
| 13374991 | 1026 | A | G | 304 | Asn | Ser |
| 13378403 | 1128 | T | C | 338 | Val | Ala |
| 13374998 | 1232 | T | C | 373 | Ser | Pro |
| 13374547 | 1285 | C | A | 390 | Ser | Ser |
| 13377992 | 1293 | T | C | 393 | Val | Ala |
| 13374997 | 1353 | A | G | 413 | Gln | Arg |
| 13381611 | 1462 | A | G | 449 | Ser | Ser |
| 13381561 | 1512 | A | G | 466 | Glu | Gly |
| 13381610 | 1550 | A | G | 479 | Arg | Gly |
| 13375006 | 1728 | A | G | 538 | Lys | Arg |
| 13375007 | 1730 | A | G | 539 | Met | Val |
| 13375784 | 2030 | A | G | 639 | Met | Val |
| 13381609 | 2034 | A | G | 640 | Lys | Arg |
| 13374546 | 2269 | T | C | 718 | Gly | Gly |
| 13374531 | 2383 | G | A | 756 | Leu | Leu |
| 13378618 | 2478 | C | A | 788 | Ser | Tyr |

NOV3b SNP Data (CG127322-01)

One polymorphic variants of NOV3b have been identified and is shown in Table 33B.

TABLE 33B
Variant of NOV3b

| | Nucleotides | | | Amino Acids | | |
|---|---|---|---|---|---|---|
| Variant | Position | Initial | Modified | Position | Initial | Modified |
| 13381583 | 106 | C | T | 20 | Gly | Gly |

NOV8b SNP Data (CG148278-01)

Four polymorphic variants of NOV8b have been identified and are shown in Table 33C. Variant 13375589 is a C to T SNP at 1642 bp of the nucleotide sequence that results in no change in the protein sequence (silent), variant 13380083 is a C to T SNP at 2785 bp of the nucleotide sequence that results in no change in the protein sequence since the SNP is not in the amino acid coding region, variant 13380084 is a G to A SNP at 2794 bp of the nucleotide sequence that results in no change in the protein sequence since the SNP is not in the amino acid coding region, and variant 13380085 is a G to A SNP at 2803 bp of the nucleotide sequence that results in no change in the protein sequence since the SNP is not in the amino acid coding region.

TABLE 33C
Variants of NOV8b

| | Nucleotides | | | Amino Acids | | |
|---|---|---|---|---|---|---|
| Variant | Position | Initial | Modified | Position | Initial | Modified |
| 13375589 | 1642 | C | T | 523 | Asp | Asp |
| 13380083 | 2785 | C | T | 0 | | |
| 13380084 | 2794 | G | A | 0 | | |
| 13380085 | 2803 | G | A | 0 | | |

NOV19a SNP Data (CG162855-01)

One polymorphic variants of NOV19a has been identified and is shown in Table 33D.

TABLE 33D
Variant of NOV19a

| | Nucleotides | | | Amino Acids | | |
|---|---|---|---|---|---|---|
| Variant | Position | Initial | Modified | Position | Initial | Modified |
| 13381580 | 1549 | G | A | 485 | Arg | Lys |

NOV20a SNP Data (CG163937-01)

Two polymorphic variants of NOV20a have been identified and are shown in Table 33E.

TABLE 33E
Variants of NOV20a

| | Nucleotides | | | Amino Acids | | |
|---|---|---|---|---|---|---|
| Variant | Position | Initial | Modified | Position | Initial | Modified |
| 13381480 | 570 | C | T | 115 | Thr | Ile |
| 13381481 | 621 | T | C | 132 | Met | Thr |

NOV22b SNP Data (CG54007-04)

Seven polymorphic variants of NOV22b have been identified and are shown in Table 33F.

TABLE 33F
Variants of NOV22b

| | Nucleotides | | | Amino Acids | | |
|---|---|---|---|---|---|---|
| Variant | Position | Initial | Modified | Position | Initial | Modified |
| 13377622 | 201 | A | T | 67 | Arg | Arg |
| 13375239 | 503 | A | G | 168 | Gln | Arg |
| 13379751 | 737 | C | T | 246 | Pro | Leu |
| 13375242 | 1075 | A | G | 359 | Met | Val |
| 13375243 | 1079 | A | G | 360 | His | Arg |
| 13375244 | 1126 | T | C | 376 | Phe | Leu |
| 13375245 | 1187 | G | A | 396 | Arg | His |

NOV23b SNP Data (CG55078-01)

Four polymorphic variants of NOV23b have been identified and are shown in Table 33G.

TABLE 33G
Variants of NOV23b

| | Nucleotides | | | Amino Acids | | |
|---|---|---|---|---|---|---|
| Variant | Position | Initial | Modified | Position | Initial | Modified |
| 13379604 | 1208 | C | T | 392 | Pro | Leu |
| 13379603 | 1218 | T | C | 395 | Ser | Ser |
| 13374975 | 1258 | T | C | 409 | Leu | Leu |
| 13379602 | 1276 | G | T | 415 | Val | Phe |

NOV24b SNP Data (CG56149-03)

One polymorphic variant of NOV24b have been identified and is shown in Table 33H.

TABLE 33H
Variant of NOV24b

| | Nucleotides | | | Amino Acids | | |
|---|---|---|---|---|---|---|
| Variant | Position | Initial | Modified | Position | Initial | Modified |
| 13381586 | 2562 | T | C | 809 | Pro | Pro |

NOV25a SNP Data (CG56216-01)

Twelve polymorphic variants of NOV25a have been identified and are shown in Table 33I.

TABLE 33I
Variants of NOV25a

| | Nucleotides | | | Amino Acids | | |
|---|---|---|---|---|---|---|
| Variant | Position | Initial | Modified | Position | Initial | Modified |
| 13376204 | 806 | T | C | 268 | Cys | Cys |
| 13381588 | 1040 | A | T | 346 | Ser | Ser |
| 13381594 | 1581 | T | C | 527 | Ser | Pro |
| 13376203 | 1746 | G | A | 582 | Gly | Ser |
| 13376202 | 1817 | C | T | 605 | Pro | Pro |
| 13375561 | 1984 | T | C | 661 | Leu | Pro |
| 13376201 | 2108 | T | C | 702 | Gly | Gly |
| 13381590 | 2182 | C | T | 727 | Ala | Val |
| 13375412 | 2193 | G | A | 731 | Ala | Thr |
| 13375563 | 2668 | A | G | 889 | Glu | Gly |
| 13375562 | 2685 | T | C | 895 | Phe | Leu |
| 13376225 | 2935 | T | C | 978 | Ile | Thr |

NOV26a SNP Data (CG56246-01)

Thirteen polymorphic variants of NOV26a have been identified and are shown in Table 33J.

TABLE 33J
Variants of NOV26a

| | Nucleotides | | | Amino Acids | | |
|---|---|---|---|---|---|---|
| Variant | Position | Initial | Modified | Position | Initial | Modified |
| 13375372 | 140 | C | T | 47 | Thr | Ile |
| 13375373 | 144 | A | T | 48 | Pro | Pro |
| 13375374 | 547 | A | G | 183 | Ile | Val |
| 13375375 | 594 | C | T | 198 | Asp | Asp |
| 13374775 | 733 | G | A | 245 | Asp | Asn |
| 13375376 | 738 | A | G | 246 | Ala | Ala |
| 13375377 | 763 | A | G | 255 | Ser | Gly |
| 13375378 | 777 | T | C | 259 | Ser | Ser |
| 13375379 | 830 | T | C | 277 | Val | Ala |
| 13375380 | 925 | A | G | 309 | Lys | Glu |
| 13375381 | 935 | A | G | 312 | Asp | Gly |
| 13375382 | 1171 | T | A | 391 | Trp | Arg |
| 13374779 | 1201 | G | A | 401 | Val | Met |

NOV28d SNP Data (CG57417-01)

Nine polymorphic variants of NOV28d have been identified and are shown in Table 33K.

TABLE 33K
Variants of NOV28d

| | Nucleotides | | | Amino Acids | | |
|---|---|---|---|---|---|---|
| Variant | Position | Initial | Modified | Position | Initial | Modified |
| 13375096 | 1371 | T | C | 420 | Cys | Arg |
| 13375098 | 1738 | A | G | 542 | Lys | Arg |
| 13375097 | 2056 | T | C | 648 | Val | Ala |
| 13374986 | 2312 | G | A | 733 | Met | Ile |
| 13374987 | 2326 | A | G | 738 | Asp | Gly |
| 13381595 | 2345 | A | G | 744 | Val | Val |
| 13374988 | 2382 | A | G | 757 | Met | Val |
| 13374989 | 2397 | C | T | 762 | Arg | Cys |
| 13381598 | 3162 | G | A | 0 | | |

NOV29b SNP Data (CG93541-01)

Two polymorphic variants of NOV29b have been identified and are shown in Table 33L.

TABLE 33L
Variants of NOV29b

| | Nucleotides | | | Amino Acids | | |
|---|---|---|---|---|---|---|
| Variant | Position | Initial | Modified | Position | Initial | Modified |
| 13379691 | 2393 | C | T | 778 | Phe | Phe |
| 13379261 | 2535 | A | C | 826 | Arg | Arg |

NOV30a SNP Data (CG93735-01)

Six polymorphic variants of NOV30a have been identified and are shown in Table 33M.

TABLE 33M
Variants of NOV30a

| | Nucleotides | | | Amino Acids | | |
|---|---|---|---|---|---|---|
| Variant | Position | Initial | Modified | Position | Initial | Modified |
| 13376144 | 332 | A | G | 64 | Lys | Lys |
| 13376142 | 436 | C | T | 99 | Ala | Val |
| 13376141 | 443 | C | T | 101 | Ala | Ala |
| 13376140 | 522 | C | T | 128 | Arg | Cys |
| 13376139 | 616 | T | C | 159 | Ile | Thr |
| 13374782 | 625 | A | G | 162 | Glu | Gly |

NOV31a SNP Data (CG93817-01)

Three polymorphic variants of NOV31a have been identified and are shown in Table 33N.

TABLE 33N
Variants of NOV31a

| | Nucleotides | | | Amino Acids | | |
|---|---|---|---|---|---|---|
| Variant | Position | Initial | Modified | Position | Initial | Modified |
| 13379394 | 74 | T | C | 18 | Phe | Ser |
| 13379395 | 322 | A | G | 101 | Met | Val |
| 13379396 | 830 | T | C | 270 | Ile | Thr |

NOV32a SNP Data (CG96859-03)

Four polymorphic variants of NOV32a have been identified and are shown in Table 33O.

TABLE 33O
Variants of NOV32a

| | Nucleotides | | | Amino Acids | | |
|---|---|---|---|---|---|---|
| Variant | Position | Initial | Modified | Position | Initial | Modified |
| 13381592 | 668 | G | A | 218 | Leu | Leu |
| 13381591 | 725 | C | T | 237 | Thr | Thr |

### Example E: SAGE Data NOV22e CarboxypeptidaseX Precursor-Like Protein

Construction of the mammalian expression vector pCEP4/Sec. The oligonucleotide primers, pSec-V5-His Forward (5′-CTCGT CCTCG AGGGT AAGCC TATCC CTAAC-3′; SEQ ID NO:518) and the pSec-V5-His Reverse (5′-CTCGT CGGGC CCCTG ATCAG CGGGT TTAAA C-3′: SEQ ID NO:519), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

Expression of CG54007-03 in human embryonic kidney 293 cells. A 2.1 kb BgIII-XhoI fragment containing the CG57004-03 sequence was subcloned into BglII-XhoI digested pCEP4/Sec to generate plasmid 356. The resulting plasmid 356 was transfected into 293 cells using the LipofectaminePlus reagent following the manufacturer's instructions (Gibco/BRL). The cell pellet and supernatant were harvested 72 h post transfection and examined for CG57004-03 expression by Western blot (reducing conditions) using an anti-V5 antibody. Table EI shows that CG57004-03 is expressed as about 95 kDa protein secreted by 293 cells.

[image]

### Example F. Method of Use

The present invention is based on the identification of biological macromolecules differentially modulated in a pathologic state, disease, or an abnormal condition or state. Among the pathologies or diseases of present interest include metabolic diseases including those related to endocrinologic disorders, cancers, various tumors and neoplasias, inflammatory disorders, central nervous system disorders, and similar abnormal conditions or states. Important metabolic disorders with which the biological macromolecules are associated include obesity and diabetes mellitus, especially obesity and Type II diabetes. It is believed that obesity predisposes a subject to Type II diabetes. In very significant embodiments of the present invention, the biological macromolecules implicated in these pathologies and conditions are proteins and polypeptides, and in such cases the present invention is related as well to the nucleic acids that encode them. Methods that may be employed to identify relevant biological macromolecules include any procedures that detect differential expression of nucleic acids encoding proteins and polypeptides associated with the disorder, as well as procedures that detect the respective proteins and polypeptides themselves. Significant methods that have been employed by the present inventors, include GeneCalling® technology and SeqCalling™ technology, disclosed respectively, in U.S. Pat. No. 5,871,697, and in U.S. Ser. No. 09/417,386, filed Oct. 13, 1999, each of which is incorporated herein by reference in its entirety. GeneCalling® is also described in Shimkets, et al., “Gene expression analysis by transcript profiling coupled to a gene database query” Nature Biotechnology 17:198-803 (1999).

The invention provides polypeptides and nucleotides encoded thereby that have been identified as having novel associations with a disease or pathology, or an abnormal state or condition, in a mammal. Included in the invention are nucleic acid sequences and their encoded polypeptides. The sequences are collectively referred to as “INDICATION nucleic acids” or “INDICATION polynucleotides” and the corresponding encoded polypeptide is referred to as a “diabetes and/or obesity polypeptide” or “diabetes and/or obesity protein”. For example, a diabetes and/or obesity nucleic acid according to the invention is a nucleic acid including a diabetes and/or obesity nucleic acid, and a diabetes and/or obesity polypeptide according to the invention is a polypeptide that includes the amino acid sequence of a diabetes and/or obesity polypeptide. Unless indicated otherwise, “diabetes and/or obesity” is meant to refer to any of the sequences having novel associations disclosed herein.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

As used herein, a “chemical composition” relates to a composition including at least one compound that is either synthesized or extracted from a natural source. A chemical compound may be the product of a defined synthetic procedure. Such a synthesized compound is understood herein to have defined properties in terms of molecular formula, molecular structure relating the association of bonded atoms to each other, physical properties such as electropherogramatic or spectroscopic characterizations, and the like. A compound extracted from a natural source is advantageously analyzed by chemical and physical methods in order to provide a representation of its defined properties, including its molecular formula, molecular structure relating the association of bonded atoms to each other, physical properties such as electropherogramatic or spectroscopic characterizations, and the like.

As used herein, a “candidate therapeutic agent” is a chemical compound that includes at least one substance shown to bind to a target biopolymer. In important embodiments of the invention, the target biopolymer is a protein or polypeptide, a nucleic acid, a polysaccharide or proteoglycan, or a lipid such as a complex lipid. The method of identifying compounds that bind to the target effectively eliminates compounds with little or no binding affinity, thereby increasing the potential that the identified chemical compound may have beneficial therapeutic applications. In cases where the “candidate therapeutic agent” is a mixture of more than one chemical compound, subsequent screening procedures may be carried out to identify the particular substance in the mixture that is the binding compound, and that is to be identified as a candidate therapeutic agent.

As used herein, a “pharmaceutical agent” is provided by screening a candidate therapeutic agent using models for a disease state or pathology in order to identify a candidate exerting a desired or beneficial therapeutic effect with relation to the disease or pathology. Such a candidate that successfully provides such an effect is termed a pharmaceutical agent herein. Nonlimiting examples of model systems that may be used in such screens include particular cell lines, cultured cells, tissue preparations, whole tissues, organ preparations, intact organs, and nonhuman mammals. Screens employing at least one system, and preferably more than one system, may be employed in order to identify a pharmaceutical agent. Any pharmaceutical agent so identified may be pursued in further investigation using human subjects.

Methods of Use of the Compositions of the Invention

The protein similarity information, expression pattern, cellular localization, and map location for the protein and nucleic acid disclosed herein suggest that this protein may have important structural and/or physiological functions characteristic of each designated protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: obesity and/or diabetes.

These materials are further useful in the generation of antibodies that bind immunospecifically to the substances of the invention for use in diagnostic and/or therapeutic methods.

The quantitative expression of various clones was assessed using microtiter plates containing RNA samples from a variety of normal and pathology-derived cells, cell lines and tissues using real time quantitative PCR (RTQ PCR). RTQ PCR was performed as described in Example C. Collections of samples in additional to those listed in Example C were assembled on plates, referred to as panels, and are described below.

Panel 1.4

The plates for panel 1.4 include 2 control wells (genomic DNA control and chemistry control) and 94 wells containing cDNA from various samples. The samples in panel 1.4 are broken into 2 classes; samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in panel 1.4 are widely available through the American Type Culture Collection, a repository for cultured cell lines. The normal tissues found on panel 1.4 are comprised of pools of samples from 2 to 5 different adult individuals derived from all major organ systems. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

A. NOV3b—Human Kynurenine Hydroxlase-Like Proteins—CG12732-01

Discovery Process—The following sections describe the study design(s) and the techniques used to identify the Kynurenine Hydroxylase-encoded protein and any variants, thereof, as being suitable as diagnostic markers, targets for an antibody therapeutic and targets for a small molecule drugs for Obesity and Diabetes.

| | |
|---|---|
| Studies: | MB.04 Genetic Models of Body Weight Regulation |
| | MB.01 SHR Rat Model of Insulin Resistance and CD36 |

Mutation

Study Statements: MB.04—A number of genetic models of obesity have been studied, most prominently in mouse and rat, but only a few causative genes have been identified. Polygenic mouse models of obesity have been evaluated by GeneCalling in order to identify the set of genes differentially expressed in obese vs. lean animals. This strategy should lead to the discovery of drug targets for the prevention and/or treatment of obesity.

MB.01—The spontaneously hypertensive rat (SHR) is a strain exhibiting features of the human Metabolic Syndrome X. The phenotypic features include visceral obesity, hypertension, increased circulating free fatty acids, hyperinsulinemia and insulin resistance. SHR rats have a mutated form of the CD36 fatty acid transporter. Decreased fatty acid transport into cells underlies the increase in circulating free fatty acids and insulin resistance. The pathophysiologic basis for hypertension is unknown but appears to be unrelated to CD36 function. Tissues were removed from adult male rats and a control strain (WKY) to identify the gene expression differences that underlie the pathologic state in the SHR rat. Tissues included subcutaneous and visceral adipose and liver.

Species #1 MB.04—C57BL (normal levels of body fat (˜18%)) and Cast/Ei (very low levels of body fat (˜8%))mouse strains

Species #2 MB.01—SHR and WKY (control) strains of rat

Kynurenine Hydroxylase: This NADPH-dependent flavin monooxygenase is a part of the pathway for oxidative degradation of tryptophan. It is the third enzyme of 4 on the dominant catabolic pathway from tryptophan to alanine (Table ##). A role for this enzyme in obesity or diabetes has not been previously reported.

In this invention, tryptophan 2,3-dioxygenase, the first enzyme in the tryptophan catabolic pathway, is dysregulated. However, the dioxygenase is not an enzyme that is amenable to high throughput screening for identification of potential inhibitors. We have therefore chosen to focus on the most screenable enzyme in the pathway, kynurenine hydroxylase.

SPECIES #1: As shown in Tables A11a, A11b, A12a and A12b, this mouse tryptophan 2,3-dioxygenase differentially expressed gene fragment from Discovery Study MB.04 was initially found to be up-regulated by 18 fold in the liver tissue of mice with normal levels of body fat (C57BL) relative to mice with very low levels of body fat (˜8%)(Cast/Ei) using CuraGen's GeneCalling™ method of differential gene expression (in the graphs, the abscissa is measured in lengths of nucleotides and the ordinate is measured as signal response). A differentially expressed mouse gene fragment migrating, at approximately 173 nucleotides in length (Table A11a—vertical line) was definitively identified as a component of the mouse tryptophan 2,3-dioxygenase cDNA. A second mouse gene fragment migrating, at approximately 331 nucleotides in length (Table A11b—vertical line) was definitively identified as a component of the mouse tryptophan 2,3-dioxygenase cDNA. The method of competitive PCR was used for conformation of the gene assessment. The chromatographic peaks corresponding to the gene fragment of the mouse tryptophan 2,3-dioxygenase are ablated when a gene-specific primer (see below) competes with primers in the linker-adaptors during the PCR amplification. The peaks at 173 nt and 331 nt in length are ablated (gray trace) in the sample from both the C57BL and Cast/Ei mice.

SPECIES #2: As shown in Tables A13a and A13b, rat differentially expressed gene fragment from Discovery Study MB.01 was found to be up-regulated by 100 fold in the liver tissue SHR rats (exhibit features of human metabolic syndrome X) relative to WKY (control strain of rat) using CuraGen's GeneCalling™ method of differential gene expression (in the graphs, the abscissa is measured in lengths of nucleotides and the ordinate is measured as signal response). A differentially expressed rat gene fragment migrating at approximately 270.6 nucleotides in length (Table A13a—vertical line) was definitively identified as a component of the tryptophan 2,3-dioxygenase cDNA. The method of competitive PCR was used for conformation of the gene assessment. The chromatographic peaks corresponding to the gene fragment of the rat tryptophan 2,3-dioxygenase are ablated when a gene-specific primer (see below) competes with primers in the linker-adaptors during the PCR amplification. The peaks at 270.6 nt in length are ablated (gray trace) in the sample from both the SHR and WKY rats.

The direct sequences of the above mouse and rat gene fragments and the gene-specific primers used for competitive PCR are indicated on the cDNA sequence of tryptophan 2,3-dioxygenase and shown below in bold. The gene-specific primers at the 5′ and 3′ ends of the fragment are in bold italics.

TABLE A1

SEQ ID NO:520 Gene Sequence
(mouse fragment from 1459 to 1631 in bold. band size: 173)

(SEQ ID NO:520)

| | |
|---|---|
| 978 | TACAGGGAGG AGCCTCGATT CCAGGTCCCT TTCCAGCTGC TGACCTCACT TATGGACATT |
| 1038 | GACACGCTCA TGACCAAATG GAGATATAAT CATGTGTGCA TGGTGCACAG AATGCTGGGC |
| 1098 | ACCAAGGCTG GCACTGGGGG ATCCTCAGGC TATCATTACC TGCGTTCAAC TGTGAGCGAC |
| 1158 | AGGTACAAGG TTTTTGTGGA TTTATTTAAC CTCTCAACAT ATCTGGTTCC CAGACACTGG |
| 1218 | GTACCAAAGA TGAATCCGAT CATTCACAAA TTCCTTTACA CAGCCGAGTA CAGTGACAGC |
| 1278 | TCTTACTTCA GCAGCGATGA ATCGGATTGA GTTCTTCTGA ACATCAGTGA AAACTACAGG |
| 1338 | ATTCTCAGTC GGCTTTTTAT AAATTTTTAT GAATACATGA TTGGTGTAAT CTATTTATAT |
| 1398 | GTGTAGTTCA GTGTTATGAT GTTTTGGTCC AATCCTGGAA AAAAGTTTAT GATCTTGCAT |
| 1458 | ATCATGATGG TGAGCGATTA GGAGGATTAA GCATTATGAT AACTGATATA GTAAAATGTT |
| 1518 | AGCATCATCG TACATATGAT AAATTCTTGC TACAACTCAA TTTACCCTGA CATTTACCTC |
| 1578 | TGTAGAACCA TTTCATATAA TTATTACCTT ATTGCTTCAT ACTTTATAAA GCTTGTTGAG |
| 1638 | CAGTTACTTT GTATTATAGA TACAATAAAT ACTACCCTTC TGTACAAAAT TTATTGAAAC |
| 1698 | AAATGTTTGA GTAATAAATT TAGTGGTTGG CTGTTCATTG CTTGTAAAAA CTCGGGAATC |
| 1758 | TTATATTTTA TGGGCGTCTT GATGAGCAGA AATCTGTCTG AAGCTTAGCC TGAGGGATAC |
| 1818 | TATAATATTA TTGCACCTGC TCCCAGAACT CAAAGGCACT TAAGAAATAT ATCACAGCAA |
| 1878 | CCCAGTTCAG CTCCCAGAAT ATTTTAACCC TGATACATTT TGAAGATGCT CTTCTTTCTT |
| 1938 | CTGTGTCAGT GTTGGCTGTC GTAAAGCATT TTGTTCCTTA CACTGTGGAC TCTGACACTT |
| 1998 | TTTCCTGCTC AAATGCTAAA GACGTGGTCA GTGCTAACAC AGGGGAATGC TGCAGAAATA |
| 2058 | AGTAGCGCTT CTATTGGAAA TTAAAAAAAA AAAAAAAAGA TCTTCCCAAC CCAA |
| | (gene length is 3021, only region from 978 to 2111 shown) |

TABLE A2

SEQ ID NO:521 Gene Sequence
(mouse fragment from 2434 to 2764 in bold. band size: 331)

(SEQ ID NO:521)

| | |
|---|---|
| 1953 | CTGTCGTAAA GCATTTTGTT CCTTACACTG TGGACTCTGA CACTTTTTCC TGCTCAAATG |
| 2013 | CTAAAGACGT GGTCAGTGCT AACACAGGGG AATGCTGCAG AAATAAGTAG CGCTTCTATT |
| 2073 | GGAAATTAAA AAAAAAAAAA AAAGATCTTC CCAACCCAAT AAACAGGTCA ACTGATTAAA |
| 2133 | CAGAAACCAT GTCCATTTGC AACAAGTACA TGATGCCTAC AGTTTATATC AGATTTGAGT |
| 2193 | CTTAGTCTTT GTTTTCTAGC TTGTTTTTTG GCTGTTGACA GCATTTAGCT GAGTTGCTGA |
| 2253 | TATGGGAAAG ACTACAAAAT ACTGGTAAAT TTTCTAAAGA TTCAAAATTA GAATTAAGAA |
| 2313 | GTTATTTTGA AGAAACAGGA AGTTCTTGAA AGAAGCACAC TATAAATCAG TCTCAACGAA |
| 2373 | ACACCATAAG TATCAGTCTT CGCTGCACTG TAATACGCAT GTAAAGTGGG ACCATCTGTT |
| 2433 | CGCTAGCTTC CACATCTTGG ATCTATCGAC TTTCCAATGT TTAATATGTA AAGGAAGAAA |
| 2493 | TACAGTATTT TTTGCAGACT TTTTGTCAGT ATTCTCTACA CAATAATAGC ATACATTGTG |
| 2553 | TTATTTTATC ACAGCTAACC TAGAAATGAC TTAAGAGTAT AAAGATGCCA GATTATATCA |
| 2613 | AAATAAATGA CACCTCACAT GTAAGAACTG ATCGTCCATG GATTTTTGGA CCTTTGGTGC |
| 2673 | TCCTGGAACT GGTATTACAG TGTTATAGAA GGAAGATGAC CATGGATTTC AACTGCACCA |
| 2733 | CTTGTGTGTA TGTAAGGTGT GCATATGTGC ACACTCACAC ATGTACTTAC ATAACACACA |
| 2793 | GTGAGGGTTA AACAGATATA AATACAGAGG AAAATACCAT GGGCTAACAG CAAAATCTCA |
| 2853 | GAAATCAGTA GGCTAAATGG TAAATGCTGA AATGGTCCTT TGTAACTATC TGTGTGGTAC |
| 2913 | ACTTCTAAGC AAACACCAGT TCCTATTTAA ATGGGGAATA CCTATTTTGT AAGCTTCATT |
| 2973 | TTCTCTCATC ATATAATAAA AAAGGCTTGT AAATAAAAAA AAAAAAAAA |
| | (gene length is 3021, only region from 1953 to 3021 shown) |

TABLE A3

SEQ ID NO:522 Gene Sequence
(rat fragment from 1 to 271 in bold. band size: 271)

(SEQ ID NO:522)

| | |
|---|---|
| 1 | GTGCACAGGA TGCTACGCAG CAAGGCTGGC ACTGGGGGAT CCTCAGGCTA TTATTATCTG |
| 61 | CGCTCAACTG TGAGCGACAG GTACAAGGTG TTCGTGGATT TATTTAACCT CTCATCGTAC |
| 121 | CTGGTTCCCC GACACTGGAT ACCAAAGATG AATCCAATCA TTCACAAGTT CCTTTACACA |
| 181 | GCTGAGTACA GCGACAGCTC CTACTTCAGC AGCGATGAAT CAGATTGAGT TTTTCTGAAC |
| 241 | ATCAGTCCAG GCTACAGGAT TCCCAGTCCA C |
| | (gene length is 271, only region from 1 to 271 shown) |

TABLE A4
Human Kynurenine Hydroxylase Gene Sequence

>CG127322-01 5000

(SEQ ID NO:523)

ntGGCACGAGCAGAAGCAACAATAATTGTGAAAAATACTTCAGCAGTTATGGACTCATCTGTCATTCAAAGGAAAAAAGT
AGCTGTCATTGGTGGTGGCTTGGTTGGCTCATTACAAGCATGCTTTCTTGCAAAGAGGAATTTCCAGATTGATGTATATG
AAGCTAGGGAAGATACTCGAGTGGCTACCTTCACACGTGGAAGAAGCATTAACTTAGCCCTTTCTCATAGAGGACGACAA
GCCTTGAAAGCTGTTGGCCTGGAAGATCAGATTGTATCCCAAGGTATTCCCATGAGAGCAAGAATGATCCACTCTCTTTC
AGGAAAAAAGTCTGCAATTCCCTATGGGACAAAGTCTCAGTATATTCTTTCTGTAAGCAGAGAAAATCTAAACAAGGATC
TATTGACTGCTGCTGAGAAATACCCCAATGTGAAAATGCACTTTAACCACAGGCTGTTGAAATGTAATCCAGAGGAAGGA
ATGATCACAGTGCTTGGATCTGACAAAGTTCCCAAAGATGTCACTTGTGACCTCATTGTAGGATGTGATGGAGCCTATTC
AACTGTCAGATCTCACCTGATGAAGAAACCTCGCTTTGATTACAGTCAGCAGTACATTCCTCATGGGTACATGGAGTTGA
CTATTCCACCTAAGAACGGAGATTATGCCATGGAACCTAATTATCTGCATATTTGGCCTAGAAATACCTTTATGATGATT
GCACTTCCTAACATGAACAAATCATTCACATGTACTTTGTTCATGCCCTTTGAAGAGTTTGAAAAACTTCTAACCAGTAA
TGATGTGGTAGATTTCTTCCAGAAATACTTTCCGGATGCCATCCCTCTAATTGGAGAGAAACTCCTAGTGCAAGATTTCT
TCCTGTTGCCTGCCCAGCCCATGATATCTGTAAAGTGCTCTTCATTTCACTTTAAATCTCACTGTGTACTGCTGGGAGAT
GCAGCTCATGCTATAGTGCCGTTTTTTGGGCAAGGAATGAATGCGGGCTTTGAAGACTGCTTGGTATTTGATCAGTTAAT
GGATAAATTCAGTAACGACCTTAGTTTGTGTCTTCCTGTGTTCTCAAGATTGAGAATCCCAGATGATCACGCCATTTCAG
ACCTATCCATGTACAATTACATAGAGATGCGAGCACATGTCAACTCAAGCTGGTTCATTTTTCAGAAGAACATGGAGAGA
TTTCTTCATGCGATTATGCCATCGACCTTTATCCCTCTCTATACAATGGTCACTTTTTCCAGAATAAGATACCATGAGGC
TGTGCAGCGTTGGCATTGGCAAAAAAAGGTGATAAACAAAGGACTCTTTTTCTTGGGATCACTGATAGCCATCAGCAGTA
CCTACCTACTTATACACTACATGTCACCACGATCTTTCCTCTGCTTGAGAAGACCATGGAACTGGATAGCTCACTTCCGG
AATACAACATGTTTCCCCGCAAAGGCCGTGGACTCCCTAGAACAAATTTCCAATCTCATTAGCAGGTGATAGAAAGGTTT
TGTGGTAGCAAATGCATGATTTCTCTGTGACCAAAATTAAGCATGAAAAAAATGTTTCCATTGCCATATTTGATTCACTA
GTGGAAGATAGTGTTCTGCTTATAATTAAACTGAATGTAGAGTATCTCTGTATGTTAATTGCAATTACTGGTTGGGGGGT
GCATTTTAAAAGATGAAACATGCAGCTTCCCTACATTACACACACTCAGGTTGAGTCATTCTAACTATAAAAGTGCAATG
ACTAAGATCCTTCACTTCTCTGAAAGTAAGGCCCTAGATGCCTCAGGGAAGACAGTAATCATGCCTTTTCTTTAAAAGAC
ACAATAGGACTCGCAACAGCATTGACTCAACACCTAGGACTAAAAATCACAACTTAACTAGCATGTTAACTGCACTTTTC
ATTACGTGAATGGAACTTACCTAACCACAGGGCTCAGACTTACTAGATAAAACCAGAAATGGAAATAAGGAATTCAGGGG
AGTTCCAGAGACTTACAAAATGAACTCATTTTATTTTCCCACCTTCAAATATAAGTATTATCATCTATCTGTTTATCGTC
TATCTATCTATCATCTATCTATCTATCTATCATCTATCTATCTATCTATCTATCTATCTATCTATCTATCTATCTATCTA
TCTCTATTTATTTATGTATTTAGAGATCAGGTCTCACTCTGTTGACCAGGCTGGAGTGCAGTGGTGAGATCTGGGTTCAC
TGCAACCTCTGCCTCCTGGGCTCAAGCAATCCTCCCACTTCAGCCTCCCAAATAGCTGGGGCTACCATGGTATTTTTCAG
TAGAGACCGGGTCTTGCCATGCTGCCCAGGCCAGTCTCAAACTCCTGGCCTCATGATGATCTGCCCACCTCAGCCTCCAA
AGTACAGGGATTAGAGTTGTGAGCCACCGCTGCCAGCCCAGAGTTACCCTCTAAAGATAAGAAAAAGGCTATTAATATCA
TACTAAGTGAAGGACAGGAAAGGGTTTTATTCATAAATTAAATGTCTACATGTGCCAGAATGGAAAGGAAACAAGGGGAG
ACAACTTTTATAGAAATACAAAGCCATTACTTTATTCAATTTCAGACCCTCAGAAGCAATTTACTAATTTATTCTTCGAC
TACATACTGCAGCAGAACCAGCAATACACTTGATTTTTAAAAGCACATTTAGTGAAATGTTTTCTTTGCTTCATCCTTCT
TTAACAGGCTGCTGAGTCACTCAGAAATCCTTCAAACATGATTAATTATGAAGATGAAACACTAGAGTCATATAAGAAAT
AAAAATTGGGCAATAAAATAAAATGATTCAGTGTTTCTTTTCTATATTGTCAATGAAAACCTTGAGTTCTAATAATCCAT
GTTCAGTTTGTAGGGAAAGAAAAAATAATTTTTTCCTTCTACCACTTTAGGTTCCTTGGCTGGGGCCCCTATAACAAAAG
ACAGATTGACAAGAGAAAAACAAACATAAATTTATTAGCGGGTATATGTAATATATATGTGGGAAATACAGGGGAATGAG
CAAATCTCAAAGAGCTGGCGTCTTAGAACTCCCTGGCTTATATAGCATCGACAAAGAACAGTAAATTTTTAGAGAAACAA
CAAAACAAAGAAAAAGAGCTTTGAGTCTGTAGGGGCAGCAATTTGGGGGAAGCAAATATATGGGAGTTTGCCTTGTAGAT
TCCTCTGGTGGTGGTCTCCAGGCTGACAAGGATTCAAAGTTGTCTCTGAAACTCCTCTTTGTCATACTGCACATATAAAA
CGTCTTTTGTTTCCAACAAGAGGATTTCTTTTTCATTCTAGAATTATCTCCTTGATAACTTGATCAGATATAGGACATGA
CACTGAATAGAGTCCAACAGTACAAAAAAAATTCAGTATGTTCTAGCTACTTCACACATGTGTACGCGACAGTTATTTTT
ACAGTAAGGTATTTTCGAGAAAAATGCATTACGTGTTTTGGAAAATAGAGTAATTTAAAAAATATATTTGAAATGAAAAT
CTCCAACACATTAGAAGATGATGATGTTAGATGCCCATCGTGTGCCACAAGTGGTTTTTTCATTATGTAAAGCACCCGTT
GAATTAAAAGAATTTGTTTTTGTTCAACCTCTTCCTGAGGCCCAAGAGCATATGGGCAATTCGGATTTCCTGCTGGACCA
CAAGGTTCTGTTGATATTACATAGAAACGGGTATTCCAGACACTTCTTATGATGAAAGTCCAAAAGTGGCATCCAATTTA
AGGCCCCATCTTTCGTTGCCATTCTTCATTCCTACAAAGGACGAACTTGGATTACATCAACTTTGGACCCATTGGTTTTG
TCGCTGTCGTCAACTGACAGTGATTCACCACTGGTGATGATAAAAATGATGGAAGAAGAGTTGAAAGTCACTTTTTTCTT
TGGCCTGTCCCCATCTTTCTGTGACATCACAATGGGTCTGATCTGCATTTCACTTCCAGCTGCTGGTAGGTCTTTAGCAG
GCCTCTGGCACCTCAGCAGTCGGAGGCACAGAAGCTGCAAAAGGGATCTTCGAAACTGGGCAGAGAAAAAATAAAGTGGA
ATATTAAGTAAAAGTTGGGCACTAATCTGGATTAACATTCGAGGAAATCAGTTGAGCTGATTTAAGTTGTTTTTTGTTTG
TTAGCAGGTGTGGATGTGGGGTTATGTGGTCATGCTCAGATCTACCTAAATCACCCCAGAGCTTTATGTCTTTTATTCAT
TCTAAATCTTATTAACCGGAATATGTAGGACCATTTCAATACCTTGTAATCCTCCAAGCTTCAATCTGCACACACTTTCT
ATGAGGGCAGGTACAACTATTAAGAGATTTTGAACATTAAGTTAGTCCACAAATATTCAGTGGGCATCTACTAGGTGACA
GCCACTGTGCTATAATTAGAGACTTTTTACTATAAGCATCAAAAACAGATAAGGCTCTTCCTGGCAGAGTTTACAGCCTG
GTGTACTTGCTAATGTCTCTTTAATTAGGTGAAGAATTTTTTTTTTCTATCGAAATTACTAATCAGTTGGGGAAAAAAAT
ACTATAGCAGACAGCACTAATGTCATCAACAAACATTGTTCTTCTCCGTGTCCTGGGTACAACATCGAATAATATTTCTT
GGCCTCCTTTCCGCTTCTCCTCTCTGCTGTTCCTCTCTACAAGAACCTGGGAGGCCAACGCCTAAAGATCATAATATCAC
AATGGAAGGAACCTAGATTCCTAAATGACTGCATAGGACAGATCCCATCTCCTCCACCCAATACATTATTAGACTGAACT
GTGACCTGAAATGAGCAATAAACTCTGTATTAATTCACTGAAATGTTGGGGTTGCTTGTTATAGTAGTCGGTCCATCATG
ACCAGTAAAACATAAATCAAAAGTTAATGTAATTGTTATCCCATTATTTAGAGCGAAATAAATGTTGAATATATGGACTT
TCTCAGATTAGGAAATACCAATTAAAAATATAATAAATAGCT

TABLE A5
Human Kynurenine Hydroxylase Protein Sequence

ORF Start: 47 ORF Stop: 1505 Frame:2
>CG127322-01-prot 486 aa

(SEQ ID NO;524)

MDSSVIQRKKVAVIGGGLVGSLQACFLAKRNFQIDVYEAREDTRVATFTRGRSINLALSHRGRQALKAVGLEDQIVSQGI
PMRARMIHSLSGKKSAIPYGTKSQYILSVSRENLNKDLLTAAEKYPNVKMHFNHRLLKCNPEEGMITVLGSDKVPKDVTC
DLIVGCDGAYSTVRSHLMKKPRFDYSQQYIPHGYMELTIPPKNGDYAMEPNYLHIWPRNTFMMIALPNMNKSFTCTLFMP
FEEFEKLLTSNDVVDFFQKYFPDAIPLIGEKLLVQDFFLLPAQPMISVKCSSFHFKSHCVLLGDAAHAIVPFFGQGMNAG
FEDCLVFDELMDKFSNDLSLCLPVFSRLRIPDDHAISDLSMYNYIEMRAHVNSSWFIPQKNMERFLHAIMPSTFIPLYTM
VTFSRIRYHEAVQRWHWQKKVINKGLFFLGSLIAISSTYLLIHYMSPRSFLCLRRPWNWIAHFRNTTCFPAKAVDSLEQI
SNLISR

The following is an alignment of the protein sequences of the human (CG127322-01), and rat (genbank accession AF056031) versions of Kynurenine Hydroxylase. Overall homology is 78%.

[image]

Kynurenine Hydroxylase; 486 Amino Acids; Locus: 1q42-q44; Intracellular Domains: Monooxygenase, Amino Acids 159-361

In addition to the human version of the Kynurenine Hydroxylase, no other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG127322-01.

TABLE A7
The variants of the Kynurenine Hydroxylase obtained from
direct cloning and/or public databases

| DNA position | strand | Alleles | AA position | AA change |
|---|---|---|---|---|
| 1090 | Plus | G:T | 348 | Leu=>Phe |
| 1105 | Plus | T:C | 353 | Asp=>Asp |
| 1126 | Plus | C:T | 360 | Ser=>Ser |
| 1153 | Plus | A:G | 369 | Ala=>Ala |
| 1161 | Plus | A:T | 372 | Asn=>Ile |
| 1243 | Plus | A:C | 399 | Thr=>Thr |

Quantitative expression analysis of clones in various cells and tissues was determined as described in Example C.

CG127322-01: Kynurenine Hydroxylase-Isoform1

Expression of gene CG127322-01 was assessed using the primer-probe set Ag4744, described in Table A8. Results of the RTQ-PCR runs are shown in Tables A9 and A10.

TABLE AS

Probe Name Ag4744

| | | | |
|---|---|---|---|
| | | | SEQ ID |
| Primers | Length | Start Position | NO: |

| | | | | |
|---|---|---|---|---|
| Forward | 5′-cagtgcttggatctgacaaagt-3′ | 22 | 486 | 527 |
| Probe | TET-5′-tcccaaagatgtcacttgtgacctca-3′-TAMRA | 26 | 508 | 528 |
| Reverse | 5′-gacagttgaataggctccatca-3′ | 22 | 544 | 529 |

TABLE A9
General_screening_panel_v1.4

| | Rel. | | Rel. |
|---|---|---|---|
| | Exp. (%) | | Exp. (%) |
| | Ag4744, | | Ag4744, |
| | Run | | Run |
| Tissue Name | 213829150 | Tissue Name | 213829150 |
| Adipose | 2.0 | Renal ca. TK-10 | 0.2 |
| Melanoma* Hs688(A).T | 0.0 | Bladder | 2.1 |
| Melanoma* Hs688(B).T | 0.0 | Gastric ca. (liver met.) NCI-N87 | 0.4 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 1.0 | Colon ca. SW480 | 0.0 |
| Squamous cell carcinoma SCC-4 | 1.1 | Colon ca.* (SW480 met) SW620 | 0.0 |
| Testis Pool | 0.8 | Colon ca. HT29 | 0.0 |
| Prostate ca.* (bone met) PC-3 | 1.0 | Colon ca. HCT-116 | 0.0 |
| Prostate Pool | 0.3 | Colon ca. CaCo-2 | 0.1 |
| Placenta | 6.3 | Colon cancer tissue | 3.0 |
| Uterus Pool | 0.0 | Colon ca. SW1116 | 0.0 |
| Ovarian ca. OVCAR-3 | 2.1 | Colon ca. Colo-205 | 0.0 |
| Ovarian ca. SK-OV-3 | 0.2 | Colon ca. SW-48 | 0.0 |
| Ovarian ca. OVCAR-4 | 0.0 | Colon Pool | 4.4 |
| Ovarian ca. OVCAR-5 | 2.8 | Small Intestine Pool | 0.4 |
| Ovarian ca. IGROV-1 | 0.9 | Stomach Pool | 4.9 |
| Ovarian ca. OVCAR-8 | 0.0 | Bone Marrow Pool | 0.4 |
| Ovary | 0.5 | Fetal Heart | 0.1 |
| Breast ca. MCF-7 | 1.2 | Heart Pool | 0.0 |
| Breast ca. MDA-MB-231 | 0.0 | Lymph Node Pool | 13.6 |
| Breast ca. BT 549 | 52.9 | Fetal Skeletal Muscle | 0.0 |
| Breast ca. T47D | 6.1 | Skeletal Muscle Pool | 0.3 |
| Breast ca. MDA-N | 0.1 | Spleen Pool | 7.1 |
| Breast Pool | 21.2 | Thymus Pool | 21.5 |
| Trachea | 0.6 | CNS cancer (glio/astro) U87-MG | 0.0 |
| Lung | 0.0 | CNS cancer (glio/astro) U-118-MG | 0.2 |
| Fetal Lung | 1.8 | CNS cancer (neuro; met) SK-N-AS | 0.0 |
| Lung ca. NCI-N417 | 0.0 | CNS cancer (astro) SF-539 | 0.1 |
| Lung ca. LX-1 | 0.0 | CNS cancer (astro) SNB-75 | 0.0 |
| Lung ca. NCI-H146 | 0.2 | CNS cancer (glio) SNB-19 | 0.1 |
| Lung ca. SHP-77 | 0.0 | CNS cancer (glio) SF-295 | 0.9 |
| Lung ca. A549 | 0.7 | Brain (Amygdala) Pool | 0.3 |
| Lung ca. NCI-H526 | 0.0 | Brain (cerebellum) | 0.0 |
| Lung ca. NCI-H23 | 0.1 | Brain (fetal) | 0.4 |
| Lung ca. NCI-H460 | 0.6 | Brain (Hippocampus) Pool | 0.4 |
| Lung ca. HOP-62 | 0.0 | Cerebral Cortex Pool | 0.2 |
| Lung ca. NCI-H522 | 0.0 | Brain (Substantia nigra) Pool | 0.0 |
| Liver | 9.3 | Brain (Thalamus) Pool | 0.0 |
| Fetal Liver | 47.3 | Brain (whole) | 0.5 |
| Liver ca. HepG2 | 0.0 | Spinal Cord Pool | 0.5 |
| Kidney Pool | 0.5 | Adrenal Gland | 0.3 |
| Fetal Kidney | 9.9 | Pituitary gland Pool | 0.0 |
| Renal ca. 786-0 | 100.0 | Salivary Gland | 0.2 |
| Renal ca. A498 | 11.0 | Thyroid (female) | 0.7 |
| Renal ca. ACHN | 1.4 | Pancreatic ca. CAPAN2 | 0.0 |
| Renal ca UO-31 | 1.5 | Pancreas Pool | 18.7 |

TABLE A10
Panel 5 Islet

| | Rel. | | Rel. |
|---|---|---|---|
| | Exp. (%) | | Exp. (%) |
| | Ag4744, | | Ag4744, |
| | Run | | Run |
| Tissue Name | 204244613 | Tissue Name | 204244613 |
| 97457_Patient-02go_adipose | 3.5 | 94709_Donor 2 AM - A_adipose | 0.0 |
| 97476_Patient-07sk_skeletal | 0.0 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placenta | 43.2 | 94712_Donor 2 AD - A_adipose | 0.0 |
| 99167_Bayer Patient 1 | 6.2 | 94713_Donor 2 AD - B_adipose | 0.0 |
| 97482_Patient-08ut_uterus | 1.0 | 94714_Donor 2 AD - C_adipose | 0.0 |
| 97483_Patient-08pl_placenta | 60.7 | 94742_Donor 3 U - A_Mesenchymal | 0.0 |
| | | Stem Cells | |
| 97486_Patient-09sk_skeletal | 0.0 | 94743_Donor 3 U - B_Mesenchymal | 0.0 |
| muscle | | Stem Cells | |
| 97487_Patient-09ut_uterus | 1.9 | 94730_Donor 3 AM - A_adipose | 0.0 |
| 97488_Patient-09pl_placenta | 47.3 | 94731_Donor 3 AM - B_adipose | 0.0 |
| 97492_Patient-10ut_uterus | 0.0 | 94732_Donor 3 AM - C_adipose | 0.0 |
| 97493_Patient-10pl_placenta | 71.7 | 94733_Donor 3 AD - A_adipose | 0.0 |
| 97495_Patient-11go_adipose | 2.5 | 94734_Donor 3 AD - B_adipose | 0.0 |
| 97496_Patient-11sk_skeletal | 0.0 | 94735_Donor 3 AD - C_adipose | 0.0 |
| muscle | | | |
| 97497_Patient-11ut_uterus | 1.8 | 77138_Liver_HepG2untreated | 0.0 |
| 97498_Patient-11pl_placenta | 17.9 | 73556_Heart_Cardiac stromal cells | 0.0 |
| | | (primary) | |
| 97500_Patient-12go_adipose | 4.4 | 81735_Small Intestine | 2.5 |
| 97501_Patient-12sk_skeletal | 0.0 | 72409_Kidney_Proximal Convoluted | 1.2 |
| muscle | | Tubule | |
| 97502_Patient-12ut_uterus | 1.2 | 82685_Small intestine_Duodenum | 0.0 |
| 97503_Patient-12pl_placenta | 100.0 | 90650_Adrenal_Adrenocortical | 4.6 |
| | | adenoma | |
| 94721_Donor 2 U - | 0.0 | 72410_Kidney_HRCE | 2.3 |
| A_Mesenchymal Stem Cells | | | |
| 94722_Donor 2 U - | 0.0 | 72411_Kidney_HRE | 0.0 |
| B_Mesenchymal Stem Cells | | | |
| 94723_Donor 2 U - | 0.0 | 73139_Uterus_Uterine smooth | 0.0 |
| C_Mesenchymal Stem Cells | | muscle cells | |

General_screening_panel_v1.4 and Panel 5 Islet: Method of Use module Summary is provided above.

Rationale for Use as a Diagnostic and/or Target for Small Molecule Drugs and Antibody Therapeutics

Alterations in expression of kynurenine hydroxylase and associated gene products function in the etiology and pathogenesis of obesity and/or diabetes. The scheme seen in Table 4 incorporates the unique findings of these discovery studies in conjunction with what has been reported in the literature. The outcome of inhibiting the action of Kynurenine Hydroxylase would be to 1) inhibit the excess production of glucose, thus ameliorating hyperglycemia in Type 2 diabetes, and 2) inhibit the synthesis of triglycerides, thus preventing excess weight gain.

Taken in total, the data indicates that an inhibitor/antagonist of Kynurenine Hydroxylase would be beneficial in the treatment of obesity and/or diabetes:

1. The carbon skeleton of tryptophan yields acetyl CoA which can be used for fatty acid synthesis.

2. SHR rats have a defective form of the CD36 membrane fatty acid transporter which prevents the intracellular transport of medium and long-chain fatty acids (see below for references): the 100-fold up-regulation of tryptophan 2,3-dioxygenase in our discovery studies may be an adaptive response in which tryptophan catabolism provides the substrate for intracellular fatty acid synthesis.

3. The side chain of tryptophan can be cleaved to yield alanine, the primary gluconeogenic amino acid.

4. Inhibitors of kynurenine hydroxylase could be an effective adjunct therapy for the treatment of obesity or the prevention of excess glucose production in Type 2 diabetes.

[image]

[image]

TABLE A14
Pathways relevant to obesity and/or diabetes etiology and
pathogenesis.

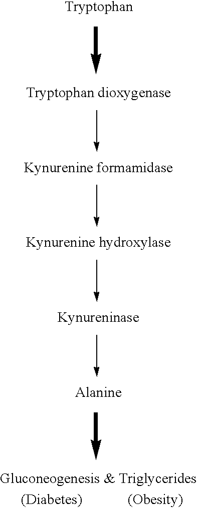

B. NOV8b (CG148278-01)—Human Long-Chain Acyl CoA Synthetase I-Like Protein

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The LONG-CHAIN ACYL COA SYNTHETASE I—encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the LONG-CHAIN ACYL COA SYNTHETASE I—encoded protein and any variants, thereof, as being suitable as diagnostic markers, targets for an antibody therapeutic and targets for a small molecule drugs for Obesity and Diabetes.

| | |
|---|---|
| Studies: | BP24.2. Diet induced obesity |

Study Statements:

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity.

| | |
|---|---|
| Studies: | MB.01 Insulin Resistance |

Study Statements:

The spontaneously hypertensive rat (SHR) is a strain exhibiting features of the human Metabolic Syndrome X. The phenotypic features include obesity, hyperglycemia, hypertension, dyslipidemia and dysfibrinolysis. Tissues were removed from adult male rats and a control strain (Wistar-Kyoto) to identify the gene expression differences that underlie the pathologic state in the SHR and in animals treated with various anti-hyperglycemic agents such as troglitizone. Tissues included sub-cutaneous adipose, visceral adipose and liver. Table B6 shows body weight distribution for diet induced obesity in spontaneously hypertensive rats.

Species #1 mouse C56B1/6

Species #2 rat WKY

Long-Chain Acyl CoA Synthetase I:

We have found long chain acyl-CoA synthetase (ACS) 2 dysregulated in multiple studies including the Diet-induced obesity study. ACS is involved in both the elongation of fatty acids, which occurs in the midrosomes, as well as beta-oxidation of fatty acids, which occurs in the mitochondria. In the genetically obese ob/ob mice the majority of the ACS activity is associated with microsomes, while in control mice it is associated with the mitochondria, suggesting that elongation of fatty acids is more predominant then beta-oxidation in the ob/obmice. Indeed, our Genecalling suggest a role for ACS in the diet-induced obesity model in fatty acid elongation.

While in mouse only one ACS is known, in human two enzymes are known, long chain acyl CoA synthetase I and long chain acyl CoA synthetase II, which are both highly homologous to the mouse ACS. Human long chain acyl CoA synthetase I is known to have a microsomal localization, and therefore is most likely the mouse orthologue involved in fatty acid elongation. Therefore, we nominate the enzyme long-chain acyl CoA synthetase I as a valuable tool to inhibit fatty acid elongation and promote beta-oxidation.

SPECIES #1 mouse (C57B1/6 obese euglycemic sd7 brown adipose tissue versus chow brown adipose tissue)

A gene fragment of the mouse long chain acyl-CoA synthetase II was initially found to be downregulated by 1.6 fold in the brown fat pad of the obese euglycemic sd7 mice relative to the chow-fed mice using CuraGen's GeneCalling™ method of differential gene expression. A differentially expressed rat gene fragment migrating at approximately 293 nucleotides in length (Table B7a.—vertical line) was definitively identified as a component of the mouse long chain acyl-CoA synthetase II cDNA in the obese euglycemic sd7 brown adipose and chow brown adipose tissue (in the graphs, the abscissa is measured in lengths of nucleotides and the ordinate is measured as signal response). The method of competitive PCR was used for conformation of the gene assessment. The chromatographic peaks corresponding to the gene fragment of the mouse long chain acyl-CoA synthetase II are ablated when a gene-specific primer (see below) which competes with primers in the linker-adaptors during the PCR amplification. The peaks at 293 nt in length are ablated in the sample from both the obese euglycemic sd7 brown adipose and chow brown adipose tissue. The altered expression in of these genes in the animal model support the role of long chain acyl-CoA synthetase I in the pathogenesis of obesity and/or diabetes.

SPECIES #2 rat (WKY Troglitazone LD10/72 h liver tissue vs. 0.02% DMSO/72 h liver tissueA gene fragment of the rat long chain acyl-CoA synthetase was initially found to be upregulated by 6.5 fold in the Troglitazone LD10/72 h treated liver tissue compared to the 0.02% DMSO/72 h untreated liver tissue of WKY rats using CuraGen's GeneCalling™ method of differential gene expression. A differentially expressed human gene fragment migrating at approximately 431 nucleotides in length (Table B8a—vertical line) was definitively identified as a component of the rat long chain acyl-CoA synthetase cDNA in the Troglitazone LD10/72 h treated liver tissue and the 0.02% DMSO/72 h untreated liver tissue of WKY rats (in the graphs, the abscissa is measured in lengths of nucleotides and the ordinate is measured as signal response). The method of competitive PCR was used for conformation of the gene assessment. The chromatographic peaks corresponding to the gene fragment of the rat long chain acyl-CoA synthetase are ablated when a gene-specific primer (see below) which competes with primers in the linker-adaptors during the PCR amplification. The peaks at 431 nt in length are ablated in the sample from both the Troglitazone LD10/72 h treated liver tissue and the 0.02% DMSO/72 h untreated liver tissue (in the graphs, the abscissa is measured in lengths of nucleotides and the ordinate is measured as signal response). The altered expression of these genes in the human cellular model support the role of long chain acyl-CoA synthetase I in the pathogenesis of obesity and/or diabetes.

Tables B7a and B7b show a differentially expressed mouse long chain acyl-CoA synthetase II gene fragment in Discovery Study BP24.02, C57B1/6 obese euglycemic sd7 brown adipose tissue versus chow brown adipose tissue.

TABLE B1
Long chain acyl-CoA synthetase II+HZ,45

Gene Sequence identified in C57B1/6 obese euglycemic sd7 brown adipose
tissue versus chow brown adipose tissue
(Identified fragment from 1806 to 2098 in bold. band size: 293).
(gene length is 2100, only region from 1325 to 2100 shown)

(SEQ ID NO:530)

| | |
|---|---|
| 1325 | CAGTGCTGAC GTTTCTGAGG ACAGCGCTCG GCTGCCAGTT CTATGAAGGC TACGGACAGA |
| 1385 | CCGAGTGCAC TGCTGGTTGC TGCCTGAGCT TGCCCGGAGA CTGGACGGCA GGCCATGTTG |
| 1445 | GAGCCCCCAT GCCTTGCAAT TATGTAAAGC TTGTGGATGT GGAAGAAATG AATTACCTGG |
| 1505 | CATCCAAGGG CGAGGGTGAG GTGTGTGTGA AAGGGGCAAA TGTGTTCAAA GGCTACTTGA |
| 1565 | AAGACCCAGC AAGAACAGCT GAAGCCCTGG ATAAAGATGG CTGGTTACAC ACGGGGGACA |
| 1625 | TTGGAAAATG GCTGCCAAAT GGCACCTTGA AGATTATCGA CAGGAAAAAG CACATATTTA |
| 1685 | AACTAGCCCA AGGAGAGTAC ATAGCACCAG AAAAGATTGA AAATATCTAC CTGCGGAGTG |
| 1745 | AAGCCGTGGC CCAGGTGTTT GTCCACGGAG AAAGCTTGCA GGCCTTTCTC ATAGCAGTTG |
| 1805 | TGGTACCCGA CGTTGAGAGC CTACCGTCCT GGGCACAGAA GAGAGGCTTA CAAGGGTCCT |
| 1865 | TCGAAGAACT GTGCAGGAAC AAGGATATCA ATAAAGCTAT CCTGGACGAC TTGTTGAAAC |
| 1925 | TTGGGAAGGA AGCCGGTCTG AAGCCATTTG AACAGGTCAA AGGCATTGCT GTGCACCCGG |
| 1985 | AATTATTTTC TATTGACAAC GGCCTTCTGA CTCCAACACT GAAGGCGAAG AGGCCAGAGC |
| 2045 | TACGGAACTA TTTCAGGTCG CAGATAGATG AACTGTACGC CACCATCAAG ATCTAA |

TABLE B2
Long chain acyl-CoA synthetase+HZ,45

gene sequence identified in WKY Troglitazone LD10/72 h liver tissue
vs. 0.02% DMSO/72 h liver tissue
(Identified fragment from 347 to 777 in bold. band size: 431)
(gene length is 3657, only region from 1 to 1257 shown)

(SEQ ID NO:531)

| | |
|---|---|
| 1 | CCAACACAGA ACTATGGAAG TCCACGAATT GTTCCGGTAT TTTCGAATGC CAGAGCTGAT |
| 61 | TGACATTCGG CAGTACGTGC GTACCCTTCC AACCAACACA CTCATGGGCT TCGGGGCTTT |
| 121 | TGCAGCGCTC ACCACCTTCT GGTATGCCAC CCGGCCGAAG GCCCTGAAGC CACCATGTGA |
| 181 | TCTGTCCATG CAGTCTGTGG AAGTAACGGG TACTACTGAG GGTGTCCGAA GATCAGCAGT |
| 241 | CCTTGAGGAC GACAAGCTCT TGCTGTACTA CTACGACGAT GTCAGAACGA TGTACGATGG |
| 301 | CTTCCAGAGG GGGATTCAGG TGTCAAATGA TGGCCCTTGT TTAGGTTCTA GAAAGCCAAA |
| 361 | CCAGCCATAT GAGTGGATTT CTTACAAACA GGTTGCAGAA ATGGCTGAGT GCATAGGCTC |
| 421 | GGCGCTGATC CAGAAGGGTT TCAAACCTTG CTCAGAGCAG TTCATCGGCA TCTTTTCTCA |
| 481 | GAACAGACCT GAGTGGGTGA CCATCGAGCA GGGGTGCTTC ACTTACTCCA TGGTGGTTGT |
| 541 | TCCGCTCTAT GACACGCTTG GAACCGACGC CATCACCTAC ATAGTGAACA AAGCTGAACT |
| 601 | CTCTGTGATT TTTGCTGACA AGCCAGAAAA AGCCAAACTC TTATTAGAAG GTGTAGAAAA |
| 661 | TAAGTTAACA CCATGCCTTA AAATCATAGT CATCATGGAC TCCTACGACA ATGATCTGGT |
| 721 | GGAACGCGGC CAGAAGTGTG GGGTGGAAAT CATCGGCCTA AAAGCTCTGG AGGATCTTGG |
| 781 | AAGAGTGAAC AGAACGAAAC CCAAGCCTCC AGAACCTGAA GATCTTGCGA TAATCTGTTT |
| 841 | CACAAGTGGA ACTACAGGCA ACCCCAAAGG AGCAATGGTC ACCCACCAAA ACATTATGAA |
| 901 | CGATTGCTCC GGTTTTATAA AAGCGACGGA GAGTGCATTC ATCGCTTCCC CAGAGGATGT |
| 961 | TCTGATATCT TTCTTGCCTC TCGCCCATAT GTTTGAGACC GTTGTAGAGT GTGTAATGCT |
| 1021 | ATGTCATGGA GCTAAGATAG GATTTTTCCA AGGAGACATC AGGCTGCTTA TGGATGACCT |
| 1081 | CAAGGTGCTT CAGCCTACCA TCTTCCCTGT GGTTCCGAGA CTGCTAAACC GGATGTTTGA |
| 1141 | CAGAATTTTT GGACAAGCAA ACACGTCAGT GAAGCGATGG CTGTTGGATT TTGCCTCCAA |
| 1201 | AAGGAAAGAG GCGGAGCTTC GCAGTGGCAT CGTCAGAAAC AACAGCCTGT GGGATAA |

Tables B8A and B8B show a differentially expressed rat long chain acyl-CoA synthetase gene fragment in Discovery Study MB.01 identified in WKY Troglitazone LD10/72 h liver tissue vs. 0.02% DMSO/72 h liver tissue.

TABLE B3
Human long chain acyl-CoA synthetase I DNA and Protein Sequence

CG148278-01.

(SEQ ID NO:532)

CGGGCAGTGACAGCCGGCGCGGATCGCGCGTCCACGGAGGAGAATCAGCTTAGAGAACTATCAACACAGGACAATGCAAG
CCCATGAGCTGTTCCGGTATTTTCGAATGCCAGAGCTGGTTGACTTCCGACAGTGCGTGACTCTTCCGACCAACACGCTT
ATGGGCTTCGGAGCTTTTTCCAGACGACTCACCACCTTCTGGCGGCCACGCCACCCAAAACCCCTGAAGCCGCCATGGCA
CCTCTCCATGCAGTCAGTGCAAGTGGCGGGTAGTGGTGGTGCACGAAGATCCGCACTACTTGACAGCGACGAGCCCTTGG
TGTATTTCTATGATGATGTTACAACATTATACGAAGGTTTCCAGAGAGGGATACAGGTGTCAAATAATGGCCCTTGTTTA
GGCTCTCGGAAACCAGACCAACCCTATGAATGGCTTTCATATAAACAGGTTGCAGAATTGTCGGAGTGCATAGGCTCAGC
ACTGATCCAGAAGGGCTTCAAGACTGCCCCAGATCAGTTCATTGGCATCTTTGCTCAAAATAGACCTGAGTGGGTGATTA
TTGAACAAGGATGCTTTGCTTATTCGATGGTGATCGTTCCACTTTATGATACCCTTGGAAATGAAGCCATCACGTACATA
GTCAACAAAGCTGAACTCTCTCTGGTTTTTGTTGACAAGCCAGAGAAGGCCAAACTCTTATTAGAGGGTGTAGAAAATAA
GTTAATACCAGGCCTTAAAATCATAGTTGTCATGGACTCGTACGGCAGTGAACTGGTGGAACGAGGCCAGAGGTGTGGGG
TGGAAGTCACCAGCATGAAGGCGATGGAGGACCTGGGAAGAGCCAACAGACGGAAGCCCAAGCCTCCAGCACCTGAAGAT
CTTGCAGTAATTTGTTTCACAAGTGGAACTACAGGCAACCCCAAAGGAGCAATGGTCACTCACCGAAACATAGTGAGCGA
TTGTTCAGCTTTTGTGAAAGCAACAGAGAATACAGTCAATCCTTGCCCAGATGATACTTTGATATCTTTCTTGCCTCTCG
CCCATATGTTTGAGAGAGTTGTAGAGTGTGTAATGCTGTGTCATGGAGCTAAAATCGGATTTTTCCAAGGAGATATCAGG
CTGCTCATGGATGACCTCAAGGTGCTTCAACCCACTGTCTTCCCCGTGGTTCCAAGACTGCTGAACCGGATGTTTGACCG
AATTTTCGGACAAGCAAACACCACCGTGAAGCGATGGCTCTTGGACTTTGCCTCCAAGAGGAAAGAAGCAGACGTTCGCA
GCGGCATCATCAGAAACAACAGCCTGTGGGACCGGCTGATCTTCCACAAAGTACAGTCGAGCCTGGGCGGAAGAGTCCGG
CTGATGGTGACAGGAGCCGCCCCGGTGTCTGCCACTGTGCTGACGTTCCTCAGAGCAGCCCTGGGCTGTCAGTTTTATGA
AGGATACGGACAGACAGAGTGCACTGCCGGGTGCTGCCTAACCATGCCTGGAGACTGGACCACAGGCCATGTTGGGGCCC
CGATGCCGTGCAATTTGATAAAACTTGGTTGGCAGTTGGAAGAAATGAATTACATGGCGTCCGAGGGCGAGGGCGAGGTG
TGTGTGAAAGGGCCAAATGTATTTCAGGGCTACTTGAAGGACCCAGCGAAAACAGCAGAAGCTTTGGACAAAGACGGCTG
GTTACACACAGGGGACATCGGAAAATGGTTACCAAATGGCACCTTGAAAATTATCGACCGGAAAAAGCACATATTTAAGC
TGGCACAAGGAGAATACATAGCCCCTGAAAAGATTGAAAATATCTACATGCGAAGTGAGCCTGTTGCTCAGGTGTTTGTC
CACGGAGAAAGCCTGCAGGCATTTCTCATTGCAATTGTGGTACCAGATGTTGAGACATTATGTTCCTGGGCCCAAAAGAG
AGGATTTGAAGGGTCGTTTGAGGAACTGTGCAGAAATAAGGATGTCAAAAAAGCTATCCTCGAAGATATGGTGAGACTTG
GGAAGGATTCTGGTCTGAAACCATTTGAACAGGTCAAAGGCATCACATTGCACCCTGAATTATTTTCTATCGACAATGGC
CTTCTGACTCCAACAATGAAGGCGAAAAGGCCAGAGCTGCGGAACTATTTCAGGTCGCAGATAGATGACCTCTATTCCAT
CATCAAGGTTTAGTGTGAAGAAGAAAGCTCAGAGGAAATGGCACAGTTCCACAATCTCTTCTCCTGCTGATGGCCTTCAT
GTTGTTAATTTTGAATACAGCAAGTGTAGGGAAGGAAGCGTTCTGTGTTTGACTTGTCCATTCGGGGTTCTTCTCATAGG
AATGCTAGAGGAAACAGAACACTGCCTTACAGTCACCTCAGTGTTCAGACCATGTTTATGGTAATACACACTTCCAAAAG
TAGCCTTAAAAATTGTAAAGGGATACTATAAATGTGCTAATTATTTGAGACTTCCTCAGTTTAAAAAGTGGGTTTTAAAT
CTTCTGTCTCCCTGTTTTTCTAATCAAGGGGTTAGGACTTTGCTATCTCTGAGATGTCTGCTACTTCGTCGAAATTCTGC
AGCTGTCTGCTGCTCTAAAGAGTACAGTGCTCTAGAGGGAAGTGTTCCCTTTAAAAATAAGAACAACTGTCCTGGCTGGA
GATCTCACAAGCGGACCAGAGATCTTTTTAAATCCCTGCTACTGTCCCTTCTCACAGGCATTCACAGAACCCTTCTGATT
CGAAGGGTTACGAAACTCATGTTCTTCTCCAGTCCCCTGTGGTTTCTGTTGGAGCATAAGGTTTCCAGTAAGCGGGAGGG
CAGATCCAACTCAGAACCATGCAGATAAGGAGCCTCTGGCAAATGGGTGCTGCATCAGAACGCGTGGATTCTCTTTCATG
GCAGATGCTCTTGGACTCGGTTCTCCAGGCCTGATTCCCCGACTCCATCCTTTTTCAGGGTTATTTAAAAATCTGCCTTA
GATTCTATAGTGAAGACAAGCATTTCAAGAAAGAGTTACCTGGATCAGCCATGCTCAGCTGTGACGCCTGATAACTGTCT
ACTTTATCTTCACTGAACCACTCACTCTGTGTAAAGGCCAACGGATTTTTAATGTGGTTTTCATATCAAAAGATCATGTT
GGGATTAACTTGCCTTTTTCCCCAAAAAATAAACTCTCAGGCAAGGCATTTCTTTTAAAGCTATTCCG

TABLE B4

>CG148278-01-prot 699 aa

(SEQ ID NO:533)

MQAHELFRYFRMPELVDFRQCVTLPTNTLMGFGAFSRRLTTFWRPRHPKPLKPPWHLSMQSVEVAGSGGARRSALLDSDE
PLVYFYDDVTTLYEGFQRGIQVSNNGPCLGSRKPDQPYEWLSYKQVAELSECIGSALIQKGFKTAPDQFIGIFAQNRPEW
VIIEQGCFAYSMVIVPLYDTLGNEAITYIVNKAELSLVFVDKPEKAKLLLEGVENKLIPGLKIIVVMDSYGSELVERGQR
CGVEVTSMKAMEDLGRANRRKPKPPAPEDLAVICFTSGTTGNPKGAMVTHRNIVSDCSAFVKATENTVNPCPDDTLISFL
PLAHMFERVVECVMLCHGAKIGFFQGDIRLLMDDLKVLQPTVFPVVPRLLNRMFDRIFGQANTTVKRWLLDFASKRKEAD
VRSGIIRNNSLWDRLIFHKVQSSLGGRVRLMVTGAAPVSATVLTFLRAALGCQFYEGYGQTECTAGCCLTMPGDWTTGHV
GAPMPCNLIKLGWQLEEMNYMASEGEGEVCVKGPNVFQGYLKDPAKTAEALDKDGWLHTGDIGKWLPNGTLKIIDRKKHI
FKLAQGEYIAPEKIENIYMRSEPVAQVFVHGESLQAFLIAIVVPDVETLCSWAQKRGFEGSFEELCRNKDVKKAILEDMV
RLGKDSGLKPFEQVKGITLHPELFSIDNGLLTPTMKAKRPELRNYFRSQIDDLYSIIKV

The following is an alignment of the protein sequences of CG148278-01 and other ACS family members ACS2 (CG93648-01), ACS 4 and ACS5. ACS2 is highly identical but is a different gene with a different chromosomal localization.

[image]

Biochemistry and Cell Line Expression

The following illustrations summarize the biochemistry surrounding the human long chain acyl-CoA synthetase I and potential assays that may be used to screen for antibody therapeutics or small molecule drugs to treat obesity and/or diabetes. Cell lines expressing the long chain acyl-CoA synthetase I can be obtained from the RTQ-PCR results shown above. These and other long chain acyl-CoA synthetase I expressing cell lines could be used for screening purposes:

ATP+a long-chain carboxylic acid+CoA=>

AMP+pyrophosphate+acyl-CoA

Subunit: monomer; Co-factor: magnesium; Inhibitor: Triacsin C (Muoio et al., 2000). Inhibits also ACS4.

Rationale for Use as a Diagnostic and/or Target for Small Molecule Drugs and Antibody Therapeutics:

The following is a summary of the findings from the discovery studies, supplementary investigations and assays that also incorporates knowledge in the scientific literature. Taken in total, the data indicates that an inhibitor/antagonist of the human LONG-CHAIN ACYL COA SYNTHETASE I would be beneficial in the treatment of obesity and/or diabetes.

In multiple genecalling studies we have found the enzyme long chain acyl-CoA synthetase (ACS) I to be dysregulated in various disease models (see below). ACS (rats) is up-regulated in liver in response to TZD treatment, while in the diet-induced obesity study ACS2 was found to be upregulated in brown adipose of DIO mice. These data suggest that the microsomal ACSs are involved in fatty acid esterification and may contribute to the obese phenotype. In human, two ACS genes exist which are 97% identical on the amino acid level. A specific inhibitor of microsomal ACS1-2 may prevent Acyl-CoA from becoming re-esterified in adipose and liver and promote beta-oxidation. This should be beneficial for the treatment of obesity.

Physical cDNA Clone Available for Expression & Screening Purposes

CG148278-01 is a full length physical clone which is the preferred cDNA, among the variants listed above, that encompasses the coding portion of the human LONG-CHAIN ACYL COA SYNTHETASE I for expression of recombinant protein from any number of plasmid, phage or phagemid vectors in a variety of cellular systems for screening purposes. The corresponding amino acid sequence is listed above (see Table B1b). Although the sequence below is the preferred isoform, any of the other isoforms may be used for similar purposes. Furthermore, under varying assay conditions, conditions may dictate that another isoform may supplant the listed isoform.

[image]

[image]

Table B9 illustrates how alterations in the expression of the human long-chain acyl CoA synthetase I and associated gene products function in the etiology and pathogenesis of obesity and/or diabetes. The scheme incorporates the unique findings of these discovery studies in conjunction with what has been reported in the literature. The outcome of inhibiting the action of the human long-chain acyl CoA synthetase I would be a way to increase lypolysis by inhibiting anti-lypolytic effects of hydrogen peroxide.

TABLE B9
Human Long-Chain Acyl CoA Synthetase I and Associated Gene
Product Pathway Relevant to the Etiology and Pathogenesis
of Obesity and/or Diabetes:

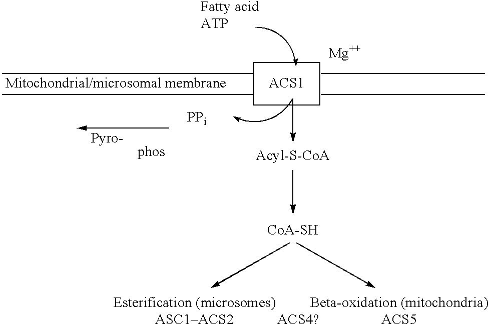

C. NOV7b (CG148010-01)—Human Diacylglycerol Acyltransferase 2-Like Protein

The present invention discloses novel associations of proteins and polypeptides and the nucleic acids that encode them with various diseases or pathologies. The proteins and related proteins that are similar to them are encoded by a cDNA and/or by genomic DNA. CuraGen Corporation identified the proteins, polypeptides and their cognate nucleic acids in certain cases. The Diacylglycerol acyltransferase 2-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the Diacylglycerol acyltransferase 2-encoded protein and any variants, thereof, as being suitable as diagnostic markers, targets for an antibody therapeutic and targets for a small molecule drugs for Obesity and/or Diabetes.

| | |
|---|---|
| Studies: | MB.04: Lean vs. Obese Genetic mouse model |
| | Diet-Induced Obesity |

Study Statements:

MB.04: A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of mouse were chosen for differential gene expression analysis because quantitative trait loci (QTL) for body weight and related traits had been reported in published genetic studies. Tissues included whole brain, skeletal muscle, visceral adipose, and liver.

DIO: The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Table C7 shows the results of this study. Tissues examined included hypothalamus, brainstem, liver, retro peritoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity.

Diacylglycerol Acyltransferase 2:

Catalyzes the reaction of 1,2-diacylglycerol+Acyl-CoA giving triacylglyceride and CoA as it's products. Palmitoyl-CoA and other long-chain acyl-CoA's can act as donors in this reaction.

SPECIES #1 Tables C6a and C6b show that two gene fragments of the mouse Diacylglycerol acyltransferase 2 were found in two different studies. The first fragment was found in the MB04 study and was up-regulated by 2.1 fold in the Adipose of the AKR/J mouse relative to the C57L/J mouse strain. The second fragment was down-regulated −1.5 fold in the Brown Adipose tissue of mice found in the Diet-Induced Obesity model comparing hyperglycemic mice of the 7 standard deviation group versus the control Chow-fed group using CuraGen's GeneCalling™ method of differential gene expression. The two differentially expressed mouse gene fragments migrated, at approximately 116.9 (MB04) and 311.6 (DIO) nucleotides in length (Tables C6a and C6b—vertical line) was definitively identified as components of the mouse Diacylglycerol acyltransferase 2 cDNA (in the graphs, the abscissa is measured in lengths of nucleotides and the ordinate is measured as signal response). The method of competitive PCR was used for conformation of the gene assessment. The electropheragramatic peaks corresponding to the gene fragment of the mouse Diacylglycerol acyltransferase 2 are ablated when a gene-specific primer (see below) competes with primers in the linker-adaptors during the PCR amplification. The peaks at 312.1 nt in length are ablated in the sample from both the NZB and SMJ mice.

Confirmatory Result—Human Diacylglycerol Acyltransferase 2 (Discovery Studies MB.04 and DIO):

The direct sequence of the 116.9 and 311.6 nucleotide-long gene fragments and the gene-specific primers used for competitive PC are indicated in italic. The gene-specific primers at the 5′ and 3′ ends of the fragment are in bold.

TABLE C1
Human Diacylglycerol acyltransferase 2 Gene Sequences

MB04: (Identified fragment from 545 to 660 in italic. band size: 116)

(SEQ ID NO:538)

TGGCATGGTACAGGTCGATGTCTTTCTGGGTCGGGTGCTCCAGCTTGGGGACAGTGATGGGCTCCCCCACGACGGTGGTG
ATGGGCTTGGAGTAGGGCACCAGCCCCCAGGTGTCAGAGGAGAAGAGGCCTCGGCCATGGAAGATGCAGGGGGCGAAACC
AATATACTTCTGGAACTTCTTCTGGACCCATCGGCCCCAGGAACCCTCCTCAAAGATCACCTGCTTGTATACCTCATTCT
CTCCAAAGGAATAAGTGGGAACCAGATCAGCTCCATGGCGCAGGGCCAGCTTCACAAAGCCTTTGCGGTTCTTCAGGGTG
ACTGCGTTCTTGCCAGGCATGGAGCTCAGGGACTCAGCTGCACCTCCCACCACGATGATGATAGCATTGCCACTCCCATT
CTTGGAGAGCAAGTAGTCTATGGTGTCTCGGTTGACAGGGCAGATGCCTCCAGACATCAGGTACTCGCGAAGCACAGGCA
TCCGGAAGTTACCAGCCAACGTAGCCAAATAGGGCCTTATGCCAGGAAACTTCTTGCTGACTTCAGTAGCCTCTGTGCTG
AAGTTACAGAAGGCACCCAGGCCCATGATGCCATGGGGGTGGTATCCAAAGATATAGTTCCTGGTGGTCAGCAGGTTGTG
TGTCTTCACCAGCTGGATGGGAAAGTAGTCTCGGAAGTAGCGCCACACGGCCCAGTTTCGCACCCACTGCGATCTCCTGC
CACCTTTCTTCGGCGTGTTCCAGTCAAATGCCAGCCAGGTGAAGTAGAGCACAGCTATCAGCCAGCAGTCTGTGCAGAAG
GTGTACATGAGGATGACACTGCAGGCCACTCCTAGCACCAGGAAGGATAGGACCCATTGTAGTACTGAGATGACCTGCAG
CTGTTTTTCCACCTTAGATCTGTTGAGCCAGGTGACAGAGAAGATGTCTTGGAGGGCTGAGAGGATGCTGGAGCCAGTGC
CCCATCGCCCAGACCCCTCGCGTGACAGGGCAGATCCTTTATTCTTGTTTTCGCTGCGGGCAGCTTCCGCCCGACGCTCA
CCCCGCAGGACCCCGGAGTAGGCGGCGATGAGGGTCT

TABLE C2
Human Diacylglycerol acyltransferase 2 Gene Sequences

DIO: (Identified fragment from 240 to 550 in italic. band Size: 311)

(SEQ ID NO:539)

GTGTCAGAGGAGAAGAGGCCTCGGCCATGGAAGATGCAGGGGGCGAAACCAATATACTTCTGGAACTTCTTCTGGACCCA
TCGGCCCCAGGAACCCTCCTCAAAGATCACCTGCTTGTATACCTCATTCTCTCCAAAGGAATAAGTGGGAACCAGATCAG
CTCCATGGCGCAGGGCCAGCTTCACAAAGCCTTTGCGGTTCTTCAGGGTGACTGCGTTCTTGCCAGGCATGGAGCTCAGG
GACTCAGCTGCACCTCCCACCACGATGATGATAGCATTGCCACTCCCATTCTTGGAGAGCAAGTAGTCTATGGTGTCTCG
GTTGACAGGGCAGATGCCTCCAGACATCAGGTACTCGCGAAGCACAGGCATCCGGAAGTTACCAGCCAACGTAGCCAAAT
AGGGCCTTATGCCAGGAAACTTCTTGCTGACTTCAGTAGCCTCTGTGCTGAAGTTACAGAAGGCACCCAGGCCCATGATG
CCATGGGGGTGGTATCCAAAGATATAGTTCCTGGTGGTCAGCAGGTTGTGTGTCTTCACCAGCTGGATGGGAAAGTAGTC
TCGGAAGTAGCGCCACACGGCCCAGTTTCGCACCCACTGCGATCTCCTGCCACCTTTCTTGGGCGTGTTCCAGTCAAATG
CCAGCCAGGTGAAGTAGAGCACAGCTATCAGCCAGCAGTCTGTGCAGAAGGTGTACATGAGGATGACACTGCAGGCCACT
CCTAGCACCAGGAAGGATAGGACCCATTGTAGTACTGAGATGACCTGCAGCTGTTTTTCCACCTTAGATCTGTTGAGCCA
GGTGACAGAGAAGATGTCTTGGAGGGCTGAGAGGATGCTGGAGCCAGTGCCCCATCGCCCAGACCCCTCGCGTGACAGGG
CAGATCCTTTATTCTTGTTTTCGCTGCGGGCAGCTTCCGCCCGACGCTCACCCCGCAGGACCCCGGAGTAGGCGGCGATG
AGGGTCTTCATGCTGAAGCCAATGCACGTCACGGCCGTGCAGAAAGCCGCCTCACGCCGCGCCCCTGACC

TABLE C3
Nucleotide and protein sequence of Human Diacylglycerol acyltransferase 2

CG148010-01

(SEQ ID NO:540)

TTCAGCCATGAAGACCCTCATAGCCGCCTACTCCGGGGTCCTGCGCGGCGAGCGTCAGGCCGAGGCTGACCGGAGCCAGC
GCTCTCACGGAGGACCCGTGTCGCGCGAGGGGTCTGGGAGATGGGGCACTGGATCCAGCATCCTCTCCGCCCTCCAGGAC
CTCTTCTCTGTCACCTGGCTCAATAGGTCCAAGGTGGAAAAGCAGCTACAGGTCATCTCAGTGCTCCAGTGGGTCCTGTC
CTTCCTTGTACTGGGAGTGGCCTGCAGTGCCATCCTCATGTACATATTCTGCACTGATTGCTGGCTCATCGCTGTGCTCT
ACTTCACTTGGCTGGTGTTTGACTGGAACACACCCAAGAAAGGTGGCAGGAGGTCACAGTGGGTCCGAAACTGGGCTGTG
TGGCGCTACTTTCGAGACTACTTTCCCATCCAGCTGGTGAAGACACACAACCTGCTGACCACCAGGAACTATATCTTTGG
ATACCACCCCCATGGTATCATGGGCCTGGGTGCCTTCTGCAACTTCAGCACAGAGGCCACAGAAGTGAGCAAGAAGTTCC
CAGGCATACGGCCTTACCTGGCTACACTGGCAGGCAACTTCCGAATGCCTGTGTTGAGGGAGTACCTGATGTCTGGAGGT
ATCTGCCCTGTCAGCCGGGACACCATAGACTATTTGCTTTCAAAGAATGGGAGTGGCAATGCTATCATCATCGTGGTCGG
GGGTGCGGCTGAGTCTCTGAGCTCCATGCCTGGCAAGAATGCAGTCACCCTGCGGAACCGCAAGGGCTTTGTGAAACTGG
CCCTGCGTCATGGAGCTGACCTGGTTCCCATCTACTCCTTTGGAGAGAATGAAGTGTACAAGCAGGTGATCTTCGAGGAG
GGCTCCTGGGGCCGATGGGTCCAGAAGAAGTTCCAGAAATACATTGGTTTCGCCCCATGCATCTTCCATGGTCGAGGCCT
CTTCTCCTCCGACACCTGGGGGCTGGTGCCCTACTCCAAGCCCATCACCACTGTTGTGGGAGAGCCCATCACCATCCCCA
AGCTGGAGCACCCAACCCAGCAAGACATCGACCTGTACCACACCATGTACATGGAGGCCCTGGTGAAGCTCTTCGACAAG
CACAAGACCAAGTTCGGCCTCCCGGAGACTGAGGTCCTGGAGGTGAACTGAGCCAGCCTTCGGGGCCAATTCCCTGGAGG
AACCAGCTGCAAATCACTTTTTTGCTCTGT

TABLE C4
ORF Start: 8 ORF Stop: 1169 Frame: 2

Human Diacylglycerol acyltransferase 2 Protein Sequence:
CG148010-01 387 aa

(SEQ ID NO:541)

MKTLIAAYSGVLRGERQAEADRSQRSHGGPVSREGSGRWGTGSSILSALQDLFSVTWLNRSKVEKQLQVI
SVLQWVLSFLVLGVACSAILMYIFCTDCWLIAVLYFTWLVFDWNTPKKGGRRSQWVRNWAVWRYFRDYFP
IQLVKTHNLLTTRNYIFGYHPHGIMGLGAFCNFSTEATEVSKKFPGIRPYLATLAGNFRMPVLREYLMSG
GICPVSRDTIDYLLSKNGSGNAIIIVVGGAAESLSSMPGKNAVTLRNRKGFVKLALRHGADLVPIYSFGE
NEVYKQVIFEEGSWGRWVQKKFQKYIGFAPCIFHGRGLFSSDTWGLVPYSKPITTVVGEPITIPKLEHPT
QQDIDLYHTMYMEALVKLFDKHKTKFGLPETEVLEVN

The following is an alignment of the protein sequences of the human DGAT2 (CG148010-01) and mouse (AF384160) versions, also included is the protein sequence for human DGAT1 (NM012079).

[image]

In addition to the human version of the Diacylglycerol acyltransferase 2 identified as being differentially expressed in the experimental study, no other variants have been identified by direct sequencing of cDNAs derived from many different human tissues and from sequences in public databases. No splice-form variants have been identified at CuraGen. The preferred variant to all those identified, to be used for screening purposes, is CG148010-01.

Biochemistry and Cell Line Expression

Table 8 summarizes the biochemistry surrounding the human diacylglycerol acyltransferase 2 enzyme. Cell lines expressing the diacylglycerol acyltransferase 2 enzyme can be obtained from the RTQ-PCR results shown above. These and other diacylglycerol acyltransferase 2 enzyme expressing cell lines could be used for screening purposes.

Findings: Diacylglycerol Acyltransferase 2 (DGAT2) is an important enzyme in the synthesis of triglycerides in both adipose, liver and muscle. DGAT2 is upregulated in the genetically obese mouse models while down regulated in brown adipose tissue in a mouse Diet Induced Obesity model. An inhibitor of DGAT2 would lead to a decrease in triacylglycerol storage. By decreasing lipid storage in particular tissues the complications associated with obesity, such as insulin resistance and Type II diabetes, may be ameliorated.

Taken in total, the data indicates that an inhibitor of the human Diacylglycerol acyltransferase 2 enzyme would be beneficial in the treatment of obesity and/or diabetes.

The sequence of Acc. No. CG148010-01 is an In silico prediction based on sequences available in CuraGen's proprietary sequence databases or in the public human sequence databases, and provided either the full length DNA sequence, or some portion thereof.

[image]

[image]

D. NOV23b—Human Secreted Carboxypeptidase HSCP1-Like Protein—CG55078-01

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The Secreted Carboxypeptidase HSCP1-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the Secreted Carboxypeptidase HSCP1-encoded protein and any variants, thereof, as being suitable as diagnostic markers, targets for an antibody therapeutic and targets for a small molecule drugs for Obesity and Diabetes.

Studies: MB.03: Type II Diabetes in Rat

Study Statements: MB.03

The GK rat was developed from the non-diabetic Wistar rat and selected over many generations on the basis of abnormal glucose tolerance. The GK rat shows mild basal hyperglycemia, marked glucose intolerance and both hepatic and peripheral insulin resistance. GK rats also demonstrate basal hyperinsulinemia and impaired insulin response to glucose. GK rats develop many of the late-term complications associated with Type 2 diabetes, including vascular disorders, nephropathy and neuropathy. Tissues were removed from adult male rats and three control strains (Wistar, Brown Norway and Fischer 344) to identify the gene expression differences that underlie the pathologic state in the GK rat model of Type II Diabetes. These specific strains of rat were chosen for differential gene expression analysis because quantitative trait loci (QTL) for diabetic traits had been reported in published genetic studies. Tissues included whole brain, skeletal muscle, visceral adipose, and liver.

Species #1: Rat Strains GK vs Brown Norway

Secreted Carboxypeptidase HSCP1:

Secreted Carboxypeptidase HSCP 1 is a new poor-characterized member of carboxypeptidase family. This class of peptidase has been implicated in hormone maturation and/or degradation of secreted peptides such as insulin, GLP-1, PACAP, the latter has a major role in metabolic processes. Some carboxypeptidases, like CPE or PC1, have been shown to be involved in development of diabetes and obesity.

SPECIES #1 (GK vs Brown Norway adipose) Tables D6a and D6b show that a gene fragment of the rat Secreted Carboxypeptidase HSCP1 was initially found to be up-regulated by 11.6 fold in the adipose tissue of the GK non-obese diabetic rat relative to normal control rat strain (Brown Norway) using CuraGen's GeneCalling™ method of differential gene expression. A differentially expressed rat gene fragment migrating, at approximately 211.7 nucleotides in length (Tables D6a and D6b—vertical line) was definitively identified as a component of the rat Secreted Carboxypeptidase HSCP1 cDNA (in the graphs, the abscissa is measured in lengths of nucleotides and the ordinate is measured as signal response). The method of competitive PCR was used for conformation of the gene assessment. The chromatographic peaks corresponding to the gene fragment of the rat Secreted Carboxypeptidase HSCP1 are ablated when a gene-specific primer (see below) competes with primers in the linker-adaptors during the PCR amplification. The peaks at 145 nt in length are ablated (gray trace) in the sample from both the GK and control rats.

The direct sequence of the nucleotide-long gene fragment and the gene-specific primers used for competitive PCR are indicated on the cDNA sequence of the Secreted Carboxypeptidase HSCP1 and are shown below in bold.

Competitive PCR Primer for the Human Secreted Carboxypeptidase HSCP1:

Physical cDNA Clone Availability for Expression and Screening Purposes: the following sequence identification is the preferred cDNA that encompasses the coding portion of human secreted carboxypeptidase HSCP1 for expression of recombinant protein from any number of plasmid, phage or phagemid vectors in a variety of cellular systems for screening purposes. Although this sequence is the preferred isoform, any of the other isoforms may be used for similar purposes. Furthermore, under varying assay conditions, those conditions may dictate that another isoform may supplant the listed isoform.

TABLE D1
Gene Sequence

(fragment from 1 to 212 in bold. band Size: 212)

(SEQ ID NO:545)

TGTACACCAATCCTAAGTCTTCAGAAACATCTGCGTTTGTCAAGTCCTATGAGAACTTAGCGTTCTACTGGATCCTAAAG
GCGGGTCACATGGTTCCTGCTGACCAAGGGGACATGGCTCTGAAGATGATGAGGCTGGTTACTCAGCAGGAGTAGCTGAG
CTGAGCTGGCCCTGGAGGCCCTGGAGGCCCTGGAGGCCCTGGAGTAGGGCCC
(gene length is 212, only region from 1 to 212 shown)

TABLE D2
Human Secreted Carboxypeptidase HSCP1 Gene Sequence

>CG55078-01 1650 nt

(SEQ ID NO:546)

GCCTGTTGCTGATGCTGCCGTGCGGTACTTGTCATGGAGCTGGCACTGCGGCGCTCTCCCGTCCCGCGGTGGTTGCTGCT
GCTGCCGCTGCTGCTGGGCCTGAACGCAGGAGCTGTCATTGACTGGCCCACAGAGGAGGGCAAGGAAGTATGGGATTATG
TGACGGTCCGCAAGGATGCCTACATGTTCTGGTGGCTCTATTATGCCACCAACTCCTGCAAGAACTTCTCAGAACTGCCC
CTGGTCATGTGGCTTCAGGGCGGTCCAGGCGGTTCTAGCACTGGATTTGGAAACTTTGAGGAAATTGGGCCCCTTGACAG
TGATCTCAAACCACGGAAAACCACCTGGCTCCAGGCTGCCAGTCTCCTATTTGTGGATAATCCCGTGGGCACTGGGTTCA
GTTATGTGAATGGTAGTGGTGCCTATGCCAAGGACCTGGCTATGGTGGCTTCAGACATGATGGTTCTCCTGAAGACCTTC
TTCAGTTGCCACAAAGAATTCCAGACAGTTCCATTCTACATTTTCTCAGAGTCCTATGGAGGAAAAATGGCAGCTGGCAT
TGGTCTAGAGCTTTATAAGGCCATTCAGCGAGGGACCATCAAGTGCAACTTTGCGGGGGTTGCCTTGGGTGATTCCTGGA
TCTCCCCTGTTGATTCGGTGCTCTCCTGGGGACCTTACCTGTACAGCATGTCTCTTCTCGAAGACAAAGGTCTGGCAGAG
GTGTCTAAGGTTGCAGAGCAAGTACTGAATGCCGTAAATAAGGGGCTCTACAGAGAGGCCACAGAGCTGTGGGGGAAAGC
AGAAATGATCATTGAACAGAACACAGATGGGGTGAACTTCTATAACATCTTAACTAAAAGCACTCCCACGTCTACAATGG
AGTCGAGTCTAGAATTCACACAGAGCCACCTAGTTTGTCTTTGTCAGCGCCACGTGAGACACCTACAACGAGATGCCTTA
AGCCAGCTCATGAATGGCCCCATCAGAAAGAAGCTCAAAATTATTCCTGAGGATCAATCCTGGGGAGGCCAGGCTACCAA
CGTCTTTGTGAACATGGAGGAGGACTTCATGAAGCCAGTCATTAGCATTGTGGACGAGTTGCTGGAGGCAGGGATCAACG
TGACGGTGTATAATGGACAGCTGGATCTCATCGTAGATACCATGGGTCAGGAGGCCTGGGTGCGGAAACTGAAGTGGCCA
GAACTGCCTAAATTCAGTCAGCTGAAGTGGAAGGCCCTGTACAGTGACCCTAAATCTTTGGAAACATCTGCTTTTGTCAA
GTCCTACAAGAACCTTGCTTTCTACTGGATTCTGAAAGCTGGTCATATGGTTCCTTCTGACCAAGGGGACATGGCTCTGA
AGATGATGAGACTGGTGACTCAGCAAGAATAGGATGGATGGGGCTGGAGATGAGCTGGTTTGGCCTTGGGGCACAGAGCT
GAGCTGAGGCCGCTGAAGCTGTAGGAAGCGCCATTCTTCCCTGTATCTAACTGGGGCTGTGATCAAGAAGGTTCTGACCA
GCTTCTGCAGAGGATAAAATCATTGTCTCTGGAGGCAATTTGGAAATTATTTCTGCTTCTTAAAAAAACCTAAGATTTTT
TAAAAAATTGATTTGTTTTGATCAAAATAAAGGATGATAATAGATATTAA

TABLE D3
Human Secreted Carboxypeptidase HSCP1 Protein Sequence

ORF Start: 34 ORF Stop: 1390 Frame: 1
>CG55078-01-prot 452 aa

(SEQ ID NO;547)

MELALRRSPVPRWLLLLPLLLGLNAGAVIDWPTEEGKEVWDYVTVRKDAYMFWWLYYATNSCKNFSELPLVMWLQGGPGG
SSTGFGNFEEIGPLDSDLKPRKTTWLQAASLLFVDNPVGTGFSYVNGSCAYAKDLAMVASDMMVLLKTFFSCHKEFQTVP
FYIFSESYGGKMAAGIGLELYKAIQRGTIKCNFAGVALGDSWISPVDSVLSWGPYLYSMSLLEDKGLAEVSKVAEQVLNA
VNKGLYREATELWGKAEMIIEQNTDGVNFYNILTKSTPTSTMESSLEFTQSHLVCLCQRHVRHLQRDALSQLMNGPIRKK
LKIIPEDQSMGGQATNVFVNMEEDFMKPVISIVDELLEAGINVTVYNGQLDLIVDTMGQEAWVRKLKWPELPKFSQLKWK
ALYSDPKSLETSAFVKSYKNLAFYWILKAGHMVPSDQGDMALKMMRLVTQQE

The following is an alignment of the protein sequences of the human (CG55078-01), rat (RISC_rat) and mouse (RISC_mouse) versions of the Secreted Carboxypeptidase HSCP1.

[image]

In addition to the human version of the Secreted Carboxypeptidase HSCP1 identified as being differentially expressed in the experimental study, no other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG55078-01.

TABLE D5

The variants of the human Secreted
Carboxypeptidase HSCP1 obtained
from direct cloning and/or public databases

| | | | | | |
|---|---|---|---|---|---|
| DNA | | | AA | AA | public |
| Position | Strand | Alleles | Position | Change | SNP # |

| | | | | | |
|---|---|---|---|---|---|
| 600 | Plus | C:T | 189 | Ile => Ile | — |
| 889 | Plus | C:T | 286 | Leu => Leu | — |
| 1258 | Plus | T:C | 409 | Leu => Leu | — |

Biochemistry and Cell Line Expression

Enzymatic activity of human Secreted Carboxypeptidase HSCP1 may be assayed by measurement the cleavage of fluorescent artificial peptide substrates, like Mca-A-P-K-(Bnp)-COOH; McA-A-G-pNF-COOH; Ac-F-ThiaF-COOH. Cell lines expressing the human Secreted Carboxypeptidase HSCP1 can be obtained from the RTQ-PCR results shown above. These and other human Secreted Carboxypeptidase HSCP1 expressing cell lines could be used for screening purposes.

Findings and Rationale for Use as a Diagnostic and/or Target for Small Molecule Drug and Antibody Therapeutics.

The following is a summary of the findings from the discovery studies, supplementary investigations and assays that also incorporates knowledge in the scientific literature. Taken in total, the data indicates that an inhibitor/antagonist of the human Secreted Carboxypeptidase HSCP1 would be beneficial in the treatment of obesity and/or diabetes.

1. Secreted Carboxypeptidase HSCP1 is up-regulated 11.7 fold in adipose of the GK diabetic rat relative to the adipose of the control strain Brown Norway rat.

2. Carboxypeptidases process, activate and/or inactivate prohormones, hormones and bio-peptides.

3. Enzymes involved in hormone maturation (e.g. CPE, PC1) have been implicated in the development of an obese phenotype.

4. Inhibition of this up-regulated carboxypeptidase may be beneficial in treating obesity.

[image]

E. NOV24b—Human Nardilysin 1-Like Protein—CG56149-03

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The NARDILYSIN 1-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the NARDILYSIN 1-encoded protein and any variants, thereof, as being suitable as diagnostic markers, targets for an antibody therapeutic and targets for a small molecule drugs for Obesity and Diabetes.

Studies: MB.03 (GK rat model for NIDDM)

Study Statements: MB.03 NIDDM is a major public health problem in Westernized nations and an increasing problem in developing countries. There are nearly 100 million people affected worldwide. Untreated NIDDM presents severe long term morbidity by not only proving to be a major risk factor for Coronary Artery Disease but also being a major contributor to kidney failure and vascular disease. The GK rat was developed from selective breeding over many generations of the non-diabetic Wistar rat colony on the basis of glucose tolerance. The GK rat shows mild basal hyperglycemia, marked glucose intolerance and both hepatic and peripheral insulin resistance. GK rats demonstrate basal hyperinsulinemia and impaired insulin response to glucose. The rat also develops many of the late-term complications associated with NIDDM including vascular disorders, nephropathy and neuropathy. Importantly, the GK rat is non-obese. Outcrosses of the GK rat with the brown Norway strain have demonstrated the evidence for possibly 7 QTL's associated with the development of NIDDM in the GK rat. A separate experiment crossing the rat to Fischer 344 rats indicated 3 QTL's associated with NIDDM, one QTL associated with body weight but not NIDDM on chromosome 7, and have weak linkage to 10 other potentially relevant loci. The purpose of our experiment is to perform QEA analysis on GK rats and compare these to each of: Wistar, Fischer 344, and Brown Norway to try to determine candidate genes for each of these QTL's. It is expected that through identification of these, (as well as through characterization of differences between other gene expression levels) we can expand our understanding of NIDDM.

NARDILYSIN 1: NRD convertase (EC 3.4.24.61) is an endopeptidase that cleaves at the N-terminus of Arg residues in dibasic sites of preproteins and propeptides; is in the insulinase family of metallopeptidases. Identified intracellularly and at the cell surface.

TABLE E1
Partial RAT NARDILYSIN 1 Gene Sequence

(fragment from 1 to 159. band size: 159)

(SEQ ID NO:551)

CGGCCGGGTTGCTCGTCTAGGAGCGGATGAATCTGAGGAGGAGGGACGGT
CTCTCAGTAATGTCGGGGACCCTGAGATCATCAAGTCTCCCAGCGATCCC
AAGCAGTACCGATACATCAAATTACAGAATGGCTTGCAGGCTCTTTTGAT
TTCAGATCT

SPECIES #1 rat (GK vs Fischer-344 Adipose)

Tables E6a and E6b show that a gene fragment of the Rat NARDILYSIN 1 was initially found to be up-regulated by 10.5 fold in the adipose tissue of GK rats relative to Fischer rats using CuraGen's GeneCalling™ method of differential gene expression. A differentially expressed rat gene fragment migrating at approximately 159 nucleotides in length (Tables E6a and E6b—vertical line) was definitively identified as a component of the rat NARDILYSIN 1 cDNA in the GK and Fischer adipose (in the graphs, the abscissa is measured in lengths of nucleotides and the ordinate is measured as signal response). The method of competitive PCR was used for conformation of the gene assessment. The chromatographic peaks corresponding to the gene fragment of the rat NARDILYSIN 1 are ablated when a gene-specific primer (see below) which competes with primers in the linker-adaptors during the PCR amplification. The peaks at 159 nt in length are ablated (gray trace) in the sample from both the GK and Fischer rats. The altered expression in of these genes in the animal model support the role of NARDILYSIN 1 in the pathogenesis of obesity and/or diabetes.

TABLE E2
Human NARDILYSIN 1 DNA and Protein Sequence

>CG56149-03 3647 nt

(SEQ ID NO:552)

AGACTGGGGTGGGGGAGGGGTTCAGGCCTGTTCCCCGCGGCTGCGGCAGCACCAGGGCCGGCCGCCACCGCCTCTAGAAC
GCGGAGGAGGTGGGTCCTGGGAAGCGGGATGTCCATCGCTCCAGCTTGGTGGTGAATGCTGAGGAGAGTCACTGTTGCTG
CAGTCTGTGCCACCCGGAGGAAGTTGTGTGAGGCCGGGCGGGACGTCGCGGCGCTCTGGGGAATCGAAACGCGGGGTCGG
TGCGAAGACTCTGCTGCTGCCAGACCCTTTCCTATTCTGGCCATGCCTGGAAGGAACAAGGCGAAGTCTACCTGCAGCTG
CCCTGACCTGCAGCCCAATGGACAGGATCTGGGCGAGAACAGCCGGGTTGCCCGTCTAGGAGCGGATGAATCTGAGGAAG
AGGGACGGAGGGGGTCTCTCAGTAATGCTGGGGACCCTGAGATCGTCAAGTCTCCCAGCGACCCCAAGCAATACCGATAC
ATCAAATTACAGAATGGCCTACAGGCACTTCTGATTTCAGACCTAAGTAATATGGAAGGTAAAACAGGAAATACAACAGA
TGATGAAGAAGAAGAGGAGGTGGAGGAAGAAGAAGAAGATGATGATGAAGATTCTGGAGCTGAAATAGAAGATGACGATG
AAGAGGGTTTTGATGATGAAGATGAGTTTGATGATGAACATGATGATGATCTTGATACTGAGGATAATGAATTGGAAGAA
TTAGAAGAGAGAGCAGAAGCTAGAAAAAAAACTACTGAAAAACAGTCTGCAGCGGCTCTTTGTGTTGGAGTTGGGAGTTT
CGCTGATCCAGATGACCTGCCGGGGCTGGCACACTTTTTGGAGCACATGGTATTCATGGGTAGTTTGAAATATCCAGATG
AGAATGGATTTGATGCCTTCCTGAAGAAGCATGGGGGTAGTGATAATGCCTCAACTGATTGTGAACGCACTGTCTTTCAG
TTTGATGTCCAGAGGAAGTACTTCAAGGAAGCTCTTGATAGATGGGCGCAGTTCTTCATCCACCCACTAATGATCAGAGA
TGCAATTGACCGTGAAGTTGAAGCTGTTGATAGTGAATATCAACTTGCAAGGCCTTCTGATGCAAACAGAAAGGAAATGT
TGTTTGGAAGCCTTGCTAGACCTGGCCATCCTATGGGAAAATTTTTTTGGGGAAATGCTGAGACGCTCAAGCATGAGCCA
AGAAAGAATAATATTGATACACATGCTAGATTGAGAGAATTCTGGATGCGTTACTACTCTTCTCATTACATGACTTTAGT
GGTTCAATCCAAAGAAACACTGGATACTTTGGAAAAGTGGGTGACTGAAATCTTCTCTCAGATACCAAACAATGGGTTAC
CCAGACCAAACTTTGGCCATTTAACGGATCCATTTGACACACCAGCATTTAACAAACTTTATAGAGTTGTTCCAATCAGA
AAAATTCATGCTCTGACCATCACATGGGCACTTCCTCCTCAACAGCAACATTACAGGGTGAAGCCACTTCATTATATATC
CTGGCTGGTTGGACATGAAGGCAAAGGCAGCATTCTTTCTTTCCTTAGGAAAAAATGCTGGGCTCTTGCACTGTTTGGTG
GAAATGGTGAGACAGGATTTGAGCAAAATTCTACTTATTCAGTGTTCAGCATTTCTATTACATTGACTGATGAGGGTTAT
GAACATTTTTATGACGTTGCTTACACTGTCTTTCTGTATTTAAAAATGCTGCAGAAGCTAGGCCCAGAAAAAAGAATTTT
TGAAGAGATTCGGAAAATTGAGGATAATGAATTTCATTACCAAGAACAGACAGATCCAGTTGAGTATGTGGAAAACATGT
GTGAGAACATGCAGCTGTACCCATTGCAGGACATTCTCACTGGAGATCAGCTTCTTTTTGAATACAAGCCAGAAGTCATT
GGTGAAGCCTTGAATCAGCTAGTTCCTCAAAAAGCAAATCTTGTTTTACTGTCTGGTGCTAATGAGGGAAAATGTGACCT
CAAGGAGAAATGGTTTGGAACTCAATATAGTATAGAAGATATTGAAAACTCTTGGGCTGAACTGTGGAATAGTAATTTCG
AATTAAATCCAGATCTTCATCTTCCAGCTGAAAACAAGTACATAGCCACGGACTTTACGTTGAAGGCTTTCGATTGCCCG
GAAACAGAATACCCAGTTAAAATTGTGAATACTCCACAAGGTTGCCTGTGGTATAAGAAAGACAACAAATTCAAAATCCC
CAAAGCATATATACGTTTCCATCTAATTTCACCGTTGATACAGAAATCTGCAGCAAATGTGGTCCTCTTTGATATCTTTG
TCAATATCCTTACGCATAACCTTGCGGAACCAGCTTATGAAGCAGATGTGGCACAGCTGGAGTATAAACTGGCAGCTGGA
GAACATGGTTTAATTATTCGAGTGAAAGGATTTAACCACAAACTACCTCTACTGTTTCAGCTCATTATTGACTACTTAGC
TGAGTTCAATTCCACACCAGCTGTCTTTACAATGATAACTGAGCAGTTGAAGAAGACCTACTTTAACATCCTCATCAAGC
CTGAGACTTTGGCCAAAGATGTACGGCTTTTAATCTTGGAATATGCCCGTTGGTCTATGATTGACAAGTACCAGGCTTTG
ATGGACGGCCTTTCCCTTGAGTCTCTGCTGAGCTTCGTCAAAGAATTCAAATCCCAGCTCTTTGTGGAGGGCCTGGTACA
AGGGAATGTCACAAGCACAGAATCTATGGATTTCCTGAAATATGTTGTTGACAAACTAAACTTCAAGCCTCTGGAGCAGG
AGATGCCTGTGCAGTTCCAGGTGGTAGAGCTGCCCAGTGGCCACCATCTATGCAAAGTGAAAGCTCTGAACAAGGGTGAT
GCCAACTCTGAAGTCACTGTGTACTACCAGTCAGGTACCAGGAGTCTAAGAGAATATACGCTTATGGAGCTGCTTGTGAT
GCACATGGAAGAACCTTGTTTTGACTTCCTTCGAACCAAGCAGACCCTTGGGTACCATGTCTACCCTACGTGTAGGAACA
CATCCGGGATTCTAGGATTTTCTGTCACTGTGGGGACTCAGGCAACCAAATACAATTCTGAAGTTGTTGATAAGAAGATA
GAAGAGTTTCTTTCTAGCTTTGACGAGAAGATTGAGAACCTCACTGAAGAGGCATTCAACACCCAGGTCACAGCTCTCAT
CAAGCTGAAGGAGTGTGAGGATACCCACCTTGGGGAGGAGGTGGATAGGAACTGGAATGAAGTGGTTACACAGCAGTACC
TCTTTGACCGCCTTGCCCACGAGATTGAAGCACTGAAGTCATTCTCAAAATCAGACCTGGTCAACTGGTTCAAGGCTCAT
AGAGGGCCAGGAAGTAAAATGCTCAGCGTTCATGTTGTTGGGTATGGGAAGTATGGACTGGAAGAGGATGGATCCCCTTC
TAGTGAGGATTCAAATTCTTCTTGTGAAGTGATGCAGCTGACCTACCTGCCAACCTCTCCTCTGCTGGCAGATTGTATCA
TCCCCATTACTGATATCAGGGCTTTCACAACAACACTCAACCTTCTCCCCTACCATAAAATAGTCAAATAAATAAACTGC
AGTCACGTTGGCCTGAAAAAAAAAAAAAAAAAAAAAAAAAAAAAAAA

TABLE E3
CG56149-03 ORF

Start: 136 ORF Stop: 3588 Frame: 1
>CG56149-03-prot 1151 aa

(SEQ ID NO:553)

MLRRVTVAAVCATRRLKCEAGRDVAALWGIETRGRCEDSAAARPFPILAMPGRNKAKSTCACPDLQPNGQDLGENSRVAR
LGADESEEEGRRGSLSNAGDPEIVKSPSDPKQYRYIKLQNGLQALLISDLSNMEGKTGNTTDDEEEEEVEEEEEDDDEDS
GAEIEDDDEEGFDDEDEFDDEHDDDLDTEDNELEELEERAEARKKTTEKQSAAALCVGVGSFADPDDLPGLAHFLEHMVF
MGSLKYPDENGFDAFLKKHGGSDNASTDCERTVFQFDVQRKYFKEALDRWAQFFIHPLMIRDAIDREVEAVDSEYQLARP
SDANRKEMLFGSLARPGHPMGKFFWGNAETLKHEPRKNNIDTHARLREFWMRYYSSHYMTLVVQSKETLDTLEKWVTEIF
SQIPNNGLPRPNFGHLTDPFDTPAFNKLYRVVPIRKIHALTITWALPPQQQHYRVKPLHYISWLVGHEGKGSILSFLRKK
CWALALFGGNGETGFEQNSTYSVFSISITLTDEGYEHFYEVAYTVFLYLKMLQKLGPEKRIFEEIRKIEDNEFHYQEQTD
PVEYVENMCENMQLYPLQDILTGDQLLFEYKPEVIGEALNQLVPQKANLVLLSGANEGKCDLKEKWFGTQYSIEDIENSW
AELWNSNFELNPDLHLPAENKYIATDFTLKAFDCPETEYPVKIVNTPQGCLWYKKDNKFKIPKAYIRFHLISPLIQKSAA
NVVLFDIFVNILTHNLAEPAYEADVAQLEYKLAAGEHGLIIRVKGFNHKLPLLFQLIIDYLAEFNSTPAVFTMITEQLKK
TYFNILIKPETLAKDVRLLILEYARWSMIDKYQALMDGLSLESLLSFVKEFKSQLFVEGLVQGNVTSTESMDFLKYVVDK
LNFKPLEQEMPVQFQVVELPSGHHLCKVKALNKGDANSEVTVYYQSGTRSLREYTLMELLVMHMEEPCFDFLRTKQTLGY
HVYPTCRNTSGILGFSVTVGTQATKYNSEVVDKKIEEFLSSFEEKIENLTEEAFNTQVTALIKLKECEDTHLGEEVDRNW
NEVVTQQYLFDRLAHEIEALKSFSKSDLVNWFKAHRGPGSKMLSVHVVGYGKYELEEDGSPSSEDSNSSCEVMOLTYLPT
SPLLADCIIPITDIRAFTTTLNLLPYHKIVK

The following is an alignment of the protein sequences of NARDILYSIN 1 and the rat version of NARDILYSIN 1. For the rat there is only a partial public sequence available.

[image]

The variants of the human NARDILYSIN 1 obtained from direct cloning and/or public databases.

In addition to the human version of the NARDILYSIN 1 identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG56149-03.

TABLE E5
Variants of human NARDILYSIN 1

| | DNA | | | | AA | |
|---|---|---|---|---|---|---|
| | Posi- | | | | Posi- | |
| SNP ID | tion | E-Value | Strand | Alleles | tion | AA Change |
| 13375411 | 590 | 2.30E−05 | Plus | A:G | 152 | Glu => Gly |
| 13375144 | 1146 | 2.30E−05 | Minus | A:C | 337 | Gly => Gly |
| 13375143 | 3048 | 9.20E−06 | Minus | G:A | 971 | Gly => Gly |

Nardilysin 1 expression results in a decreased level of active insulin.

[image]

F. NOV25a—Human SERCA 3-Like Protein—CG56216-01

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The Serca 3-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process

The following sections describe the study design(s) and the techniques used to identify the Serca 3-encoded protein and any variants, thereof, as being suitable as diagnostic markers, targets for an antibody therapeutic and targets for a small molecule drugs for obesity and diabetes.

| | | |
|---|---|---|
| Studies: | MB.11 | Rat Insulin Secretion |

Study Statements: MB.11—The regulation of insulin secretion is critical to the control of serum glucose concentrations. Alterations in the secretion of insulin are central to the etiology, pathogenesis and consequences of both Type I and Type II diabetes. This study was designed to determine the role of gene expression in regulating insulin secretion from rat pancreatic beta cell lines derived from the heterogeneous rat INS-1 insulinoma. The rat insulinoma cell line INS-1 was transfected with the plasmid pCMV8/INS/IRES/Neo. The plasmid expresses the human insulin gene and the neo selectable marker under the control of the CMV promoter. Stable clones expressing these genes were isolated and described in Hohmeier, H E, Mulder, H., Chen, G., Prentki, M., Newgard, C B: Isolation of INS-1 derived cell lines with robust K ATP channel-dependent and independent glucose stimulated insulin secretion. Diabetes 49: 424-430, 2000.

TABLE F1

| | | Poor Insulin | Good Insulin |
|---|---|---|---|
| Phenotypes Of The Cell Lines | | Secretion | Secretion |
| Glucagon Expression | Negative | 832/1 | 832/13 |
| | | 832/2 | 833/15 |
| | Positive | 834/105 | |
| | | 834/112 | |

Species #1: Rat Insulinoma Cell Line INS-1

Serca 3: Serca3 is a sarcoplasmic/endoplasmic reticulum calcium ATPase 3. It is a magnesium dependent enzyme that catalyzes the hydrolysis of ATP coupled with the transport of the calcium. This enzyme transports calcium ions from the cytosol into the sarcoplasmic/endoplasmic reticulum and has a central role in intracellular calcium signaling.

SPECIES #1 A gene fragment of the rat Serca 3 was initially found to be up-regulated by 9 fold in the glucagon negative/good insulin secreting rat INS-1 cell line relative to glucagon negative/poor insulin secreting rat INS-1 cell line using CuraGen's GeneCalling™ method of differential gene expression. A differentially expressed rat gene fragment migrating, at approximately 51.9 nucleotides in length (Table F8a—vertical line) was definitively identified as a component of the rat Serca 3 cDNA (in the graphs, the abscissa is measured in lengths of nucleotides and the ordinate is measured as signal response). The method of competitive PCR was used for conformation of the gene assessment. The electropherogram peaks corresponding to the gene fragment of the rat Serca 3 are ablated when a gene-specific primer (see below) competes with primers in the linker-adaptors during the PCR amplification. The peaks at 51.9 nt in length are ablated in the sample from both the good and poor insulin secreting rat insulinoma cell lines.

Competitive PCR Primer for the Rat Serca 3

The direct sequence of the 51.9 nucleotide-long gene fragment and the gene-specific primers used for competitive PCR are indicated on the complete cDNA sequence of the Serca 3 and shown below in bold.

TABLE F2
Human Serca 3 Gene Sequence

Gene Sequence (fragment from 3766 to 3817 in bold, band size: 52)

(SEQ ID NO:556)

| | |
|---|---|
| 3285 | CCAGGGTCCT GTCTCCATTC CTGTCCTCCC TACAGCTCTG GCCCAGAAGT TGAGCCCAGG |
| 3345 | AGGGGAAGCT GAGAAGCCAG AGCTGCCTGA AGGGCCACCA CCCATTTCCC CCACAGATCT |
| 3405 | GCAGCCTTCC CCTGCTCGGA GGTGGGCATT TGCTTGGTAG CTAGTGCCTC TGCCCTGATG |
| 3465 | GAGGGCTCAT GGGGGCCGTC TTACTGACTC TGACCTCTTG CTTAGTTTGG GTCTGGAGCC |
| 3525 | TGGTCAGCTC TGGGAAGGAG GAGTCCGAGG GGACCATCTG GGTCCAGCTG TGAACATGAG |
| 3585 | GGGCAGCCCC TTCCACTTGG CTCAGCTTCC ACCAAGTCCA CTCCTGTGTC TGTTTATGTA |
| 3645 | TCTGCTGGCC CCAGGGAGTT GAAGGATCAC AGACAGATAG GAACATAAGG AGCAGCGGGG |
| 3705 | GCAGGCCTGG ACAAGGACTC CTTTCCCAGG AGTCAGCCTC CACTGGCTGG CTGGGCTCAG |
| 3765 | CACTAGTCCC ACCTTGAGGC TCACTTCCTC GGCTCAGGTT GGCTCAGGGA TCCTAACTTT |
| 3825 | ACAGTCCATG CCCCTGGTGC CTGAGACTCC AGGCATCCCT GGGTCCATGT CAGCTTCTCC |
| 3885 | TGCCACGAAG CCTGGGGTGA TACCGTGTCA CTTGCTGCAG GGCTGGGTGA TTCTAAACCT |
| 3945 | CCTGGACCCC TGGCATTACT CTTTGCCCTC TTTTCCTATC ATGCATGTCT GAGTCAGAGA |
| 4005 | GATGTCACTA GGGAGTGACT CCACAATCCT CCCCTACCTC CCCACTGAAA GGAAGCATCT |
| 4065 | GATGGGGGTC TATCAGATGA ATGTGTATTG GTCTTTGGGA TCTTTTTTGC CTCTTAACCC |
| 4125 | TGCTGTTGCT CCTTTGACAA AAGCTAGCTA AGCATCATGG GAAACGGAGA AAGCGCCTGT |
| 4185 | CAGTGTGACT TAGCTCTTCC CTGACTGTGT ACAATATGAT TATTTTATAT GTAAATCAAG |
| 4245 | GTTCACATCA CTGTCCTGAC ACCTGGTAGC AAAAGTCCCC TCAGCCTACC CAG |
| | (gene length is 4497, only region from 3285 to 4297 shown) |

TABLE F3
Human Serca 3 Gene Sequence

>CG56216-01 3147 nt

(SEQ ID NO:557)

GCATGGAGGCCGCGCATCTGCTCCCGGCCGCCGACGTGCTGCGCCACTTCTCGGTGACAGCCGAGGGCGGCCTGAGCCCG
GCGCAGGTGACCGGCGCGCGGGAGCGCTACGGCCCCAACGAGCTCCCGAGTGAGGAAGGGAAGTCCCTGTGGGAGCTGGT
GCTGGAACAGTTTGAGGACCTCCTGGTGCGCATCCTGCTGCTGGCTGCCCTTGTCTCCTTTGTCCTGGCCTGGTTCGAGG
AGGGCGAGGAGACCACGACCGCCTTCGTGGAGCCCCTGGTCATCATGCTGATCCTCGTGGCCAACGCCATTGTGGGCGTG
TGGCAGGAACGCAACGCCGAGAGTGCCATCGAGGCCCTGAAGGAGTATGAGCCTGAGATGGGCAAGGTGATCCGCTCGGA
CCGCAAGGGCGTCCAGAGGATCCGTGCCCGGGACATCGTCCCAGGGGACATTGTAGAAGTGGCAGTGGGGGACAAAGTGC
CTGCTGACCTCCGCCTCATCGAGATCAACTCCACCACGCTGCGAGTGGACCAGTCCATCCTCACGGGTGAATCTGTGTCC
GTGACCAAGCACACAGAGGCCATCCCAGACCCCAGAGCTGTGAACCAGGACAAGAAGAACATGCTGTTTTCTGGCACCAA
TATCACATCGGGCAAAGCGGTGGGTGTGGCCGTGGCCACCGGCCTGCACACGGAGCTGGGCAAGATCCGGAGCCAGATGG
CGGCAGTCGAGCCCGAGCGGACGCCGCTGCAGCGCAAGCTGGACGAGTTTGGACGGCAGCTGTCCCACGCCATCTCTGTG
ATCTGTGTGGCCGTGTGGGTCATCAACATCGGCCACTTCGCCGACCCGGCCCACGGTGGCTCCTCGCTGCGTCGCGCTGT
CTACTACTTCAAGATCGCCGTGGCCCTGGCGGTGGCGGCCATCCCCGAGGGCCTCCCGGCTGTCATCACTACATGCCTGG
CACTGGGCACGCGCCGCATGGCACGCAAGAACCCCATCGTGCGAAGCCTGCCGTCCGTGGAGACCCTGGGCTGCACCTCA
GTCATCTGCTCCCACAAGACGCGCACGCTCACCACCAATCAGATGTCTGTCTGCCGGATGTTCGTGGTAGCCCACGCCGA
TGCGGGCTCCTGCCTTTTGCACGAGTTCACCATCTCGGGTACCACGTATACCCCCGAGCGCGAAGTGCGGCAGGGGGATC
AGCCTGTGCGCTGCGGCCAGTTCGACGGGCTGGTGGAGCTGGCCACCATCTGCGCCCTGTGCAACGACTCGGCTCTGGAC
TACAACGAGGCCAACGGTGTGTATGAGAAGGTCGGAGAGGCCACGGAGACAGCTCTGACTTGCCTGGTGGAGAAGATGAA
CGTGTTCGACACCGACCTGCACGCTCTGTCCCGGGTGGAGCGAGCTGGCGCCTCTAACACGGTCATCAAGCAGCTGATGC
GGAAGGAGTTCACCCTGGAGTTCTCCCGAGACCGGAAATCCATGTCCCTGTACTGCACGCCCACCCGCCCTCACCCTACT
GGCCAGGGCAGCAAGATGTTTGTGAAGGGGGCTCCTGAGAGTGTGATCGAGCGCTGTAGCTCAGTCCGCGTCGGGAGCCG
CACAGCACCCCTGACCCCCACCTCCAGGGAGCAGATCCTGGCAAAGATCCGGGATTGGGGCTCACGCTCACACACGCTGC
GCTGCCTGGCACTGGCCACCCGGGACGCGCCCCCAAGGAAGGACGACATGGAGCTGGACGACTGCGGCAAGTTTGTGCAG
TACGAGACGGACCTGACCTTCGTGGOCTGCGTACCCATGCTGGACCCGCCGCGACCCGAGGTGGCTGCCTGCATCACACG
CTGCTACCACGCGGGCATCCGCGTGGTCATGATCACGGGGGATAACAAAGGCACTGCCGTGGCCATCTGCCGCACGCTTG
GCATCTTTGGGGACACGGAAGACGTGGCGGGCAAGGCCTACACGGGCCGCGAGTTTGATGACCTCAGCCCCGAGCAGCAG
CGCCAGGCCTGCCGCACCGCCCGCTGCTTCGCCCGCGTGGAGCCCGCACACAAGTCCCGCATCGTGGAGAACCTGCAGTC
CTTTAACGAGATCACTGCTATGACTGGTGATGGAGTGAACGACGCACCAGCCCTGAAGAAAGCAGAGATCGGCATCGCCA
TGGGCTCAGGCACGGCCGTGGCCAACTCGGCGGCAGAGATGGTGCTGTCAGATGACAACTTTGCCTCCATCGTGGCTGCG
GTGGAGGAGGGCCGGGCCATCTACAGCAACATGAAGCAATTCATCCGCTACCTCATCTCCTCCAATGTTGGCGAGGTCGT
CTGCATCTTCCTCACGGCAATTCTGGGCCTGCCCGAAGCCCTGATCCCTGTGCAGCTGCTCTGGGTGAACCTGGTGACAG
ACGGCCTACCTGCCACCGCTCTGCGCTTCAACCCGCCAGACCTGGACATCATGGAGAAGCTGCCCCGGAGCCCCCGAGAA
GCCCTCATCAGTGGCTGGCTCTTCTTCCGATACCTGGCTATCGGAGTGTACGTAGGCCTGGCCACAGTGGCTGCCGCCAC
CTGGTCGTTTGTGTATCACGCCGAGGGACCTCACATCAACTTCTACCAGCTGAGGAACTTCCTGAAGTGCTCCGAAGACA
ACCCGCTCTTTGCCGGCATCGACTGTGAGGTGTTCGAGTCACGCTTCCCCACCACCATGGCCTTGTCCGTGCTCGTGACC
ATTGAAATGTGCAATGCCCTCAACAGCGTCTCGGAGAACCAGTCGCTGCTGCGGATGCCGCCCTGGATGAACCCCTGGCT
GCTGGTGGCTGTGGCCATGTCCATGGCCCTGCACTTCCTCATCCTGCTCGTGCCGCCCCTGCCTCTCATTTTCCAGGTGA
CCCCACTGAGCCGGCGCCAGTCGGTGGTGGTGCTCCAGATATCTCTGCCTGTCATCCTGCTGGATGAGGCCCTCAAGTAC
CTGTCCCGGAACCACATGCACGAAGAAATGAGCCAGAAGTGAGCGCTGCGAACAGACTGGAGTCTCCGGTGTGTACCTCA
GACTGATGGTGCCCATGTGTTCGCCTCCGCCCCCCACCCTTGCCACCACACTCGCCCACTTGCCCACCGGGTCCCGCCGG
ATAAATGACAGGCCCGAGGTCAGAATG

TABLE F4
Human Serca 3 Protein Sequence

ORF Start: 3 ORF Stop: 3000 Frame: 3
>CG56216-01-prot 999 aa

(SEQ ID NO:558)

MEAAHLLPAADVLRHFSVTAEGGLSPAQVTGARERYGPNELPSEEGKSLWELVLEQPEDLLVRILLLAALVSFVLAWFEE
GEETTTAFVEPLVIMLILVANAIVGVWQERNAESAIEALKEYEPEMGKVIRSDRKGVQRIRARDIVPGDIVEVAVGDKVP
ADLRLIEIKSTTLRVDQSILTGESVSVTKHTEAIPDPRAVNQDKKNMLFSGTNITSGKAVGVAVATGLHTELGKIRSQMA
AVEPERTPLQRKLDEFGRQLSHAISVICVAVWVINIGHFADPAHGGSWLRGAVYYFKIAVALAVAAIPEGLPAVITTCLA
LGTRRMARKNAIVRSLPSVETLGCTSVICSDKTGTLTTNQMSVCRMFVVAEADAGSCLLHEFTISGTTYTPEGEVRQGDQ
PVRCGQFDOLVELATICALCNDSALDYNEAKGVYEKVGEATETALTCLVEKMNVFDTDLQALSRVERAGACNTVIKQLMR
KEFTLEFSRDRKSMSVYCTPTRPHPTGQGSKMFVKGAPESVIERCSSVRVGSRTAPLTPTSREQILAKIRDWGSGSDTLR
CLALATRDAPPRKEDMELDDCGKEVQYETDLTFVGCVGMLDPPRPEVAACITRCYQAGIRVVMITGDNKGTAVAICRRLG
IFGDTEDVAGKAYTCREFDDLSPEQQRQACRTARCFARVEPAHKSRIVENLQSFNEITAMTGDGVNDAPALKKAEIGIAM
GSGTAVARSAAEMVLSDDNFASIVAAVEEGRAIYSNMKQFIRYLISSNVGEVVCIFLTAILGLPEALIPVQLLWVNLVTD
GLPATALGFNPPDLDIMEKLPRSPREALISGWLFFRYLAIGVYVGLATVAAATWWFVYDAEGPHINFYQLRNFLKCSEDN
PLFAGIDCEVFESRFPTTMALSVLVTIEMCNALNSVSENQSLLRMPPWMWNPWLLAVAMSMALHFLILLVPPLPLIFQVT
PLSGRQWVVVLQISLPVILLDEALKYLSRNHMHEEMSQK

The following is an alignment of the protein sequences of the human (CG56216-01), rat and mouse versions of the Serca 3.

[image]

In addition to the human version of the Serca 3 identified as being differentially expressed in the experimental study, no other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG56216-01.

TABLE F6

The variants of the human Serca 3 obtained
from direct cloning and/or public databases

| | | | | | |
|---|---|---|---|---|---|
| DNA | | | AA | AA | public |
| Position | Strand | Alleles | Position | Change | SNP # |

| | | | | | |
|---|---|---|---|---|---|
| 1513 | Plus | A:G | 504 | His => Arg | — |
| 1984 | Plus | T:C | 661 | Leu => Pro | — |
| 2193 | Minus | G:A | 731 | Ala => Thr | — |
| 2623 | Minus | T:C | 874 | Leu => Pro | — |
| 2668 | Minus | A:G | 889 | Glu => Gly | — |
| 2685 | Minus | T:C | 895 | Phe => Leu | — |

The probe and primers were designed on the 3′ untranslated region of SERCA3, which is not included in CG56216-01. Below is a clustalW (Table F7) of the sequence submitted for the development of RTQ-PCR (“human SERCA3 submitted for RTQ-PCR”) and CG56216-01. The positions of the primers and probe in table AA correspond to the positions in the RTQ-PCR sequence. Since it both concerns the gene of SERCA3 the primers will recognize the gene encoding CG56216-01.

[image]

[image]

[image]

Rationale for Use as a Diagnostic and/or Target for Small Molecule Drugs and Antibody Therapeutics.

The following is a summary of the findings from the discovery studies, supplementary investigations and assays that also incorporates knowledge in the scientific literature. Taken in total, the data indicates that an inhibitor/antagonist of the human Serca3 would be beneficial in the treatment of obesity and/or diabetes.

Insulin secretion by the pancreatic beta cell is acutely stimulated by an influx of calcium through voltage-gated calcium channels in the plasma membrane. Restoration of intracellular calcium homeostasis is accomplished, in part, by uptake into calcium storage sites, including the endoplasmic reticulum (ER). SERCA3 is an ATPase that mediates calcium transport into the ER. It is upregulated 7-fold in good insulin-secreting insulinoma cell lines versus poor insulin-secreting insulinoma cell lines (MB.11). Insulin secretagogues that stimulate intracellular calcium influx, also elevate calcium levels in the ER (Maechler, P. et al. Secretagogues modulate the calcium concentration in the endoplasmic reticulum of insulin-secreting cells. J Biol Chem 274:12583-12592, 1999). Thus, SERCA3-mediated calcium uptake into the ER optimizes both beta cell calcium homeostasis and the insulin secretory process. Finally, SERCA3 is downregulated in islet tissue of the diabetic GK rat, further supporting an important role for SERCA3 in insulin secretion (Varadi, A. et al. Isoforms of endoplasmic reticulum Ca++-ATPase are differentially expressed in normal and diabetic islets of Langerhans. Biochem J 319:521-527, 1996). The combined data suggest that activation of SERCA3 will promote beta cell insulin secretion and be an effective treatment for the beta cell secretory defect in Type 2 diabetes.

[image]

G. NOV26a—Olfactory Receptor-Like Protein—CG56230

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by Curagen Corporation in certain cases. The Human Neutral Amino Acid Transporter B-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

TABLE G1
Consensus DNA Sequence, CG56230-01

CG56230-01: Olfactory Receptor Isoform 1
Acc. No.: GMAC072059_H
>CG56230-01 911 nt

(SEQ ID NO:564)

ATCATTTCCTCCAGAACCAGGGTCTACTCATTAATTTCCTCATAGCTCCATGCACCTCTGTATTCCTCAGGCTGTAGATG
ATAGGATTGGGCATGGGGGTGACTATGCCATAGAACAGGGCAATCAGTTTGTCAAAAGCAGAGTCTTTGGACTTTGCCTT
CATGTACATGAAGAGGATTGTCCCATAAAACACAATCACCACTGTCATGTGGGCTGAGCAGGTGGAAAAGGCCTTTTTCC
TTCCTTCAGCTGAATTGATTCTTAGTACAGTAGAAAGGATAAAGATGTAGGAGATACAAATCAGCAGTAATGGAGAAAAC
AAAAATATTACATTGCCCAACATTATAATAATCTCATTCAAGGAAGTATCTGTGCAAGCCAGCTTGACAAAGGCCAATAT
TTCACAAACAAAATGATTGATGACATTTTTTCCACAGAAGGGTAACCGTATTGCAAGAACAGTTTCTGTCAATGAGTTGA
GAAAGCCTAGTCCCCAAGAGACAGCCACCATCTGAATACAAAGTGCCTTGCCCATGATGATGGGATATCTCAGAGGGTTG
CAGATGGCTACATAACGGTCATATGCCATCACTGCTAGAAGCACACACTTGGTGGATCCCATAGTGTAAGAGACAGACAT
TTGAATCACACATCTAGTGAAGGAAATGGTTTTCTTCTCTGATGGGAAGTGTATCAGCATTGAGGGGATGGAGGAGGATG
TGTACCAAATGTCTAGGAAGGAGAGATTCCCAAGGAAGAAGTACATGGGTGTGTGGAGACGAGCATCCAGGAGTGTCAGA
ATGATCAAGGTGCCATTCCCTAGGAGAATCACCAGGTACATCACTAAGCACATCACGAAAAGGAATTTTTCAGCTCTTGG
GTACCCTGAAAGTCCTTGCAGAATGAACTCT

TABLE G2
Protein Sequence:

ORF Start: 2 ORF Stop: 908 Frame: −2
>CG56230-01-prot 302 aa

(SEQ ID NO:565)

EFILQGLSGYPRAEKFLFVMCLVMYLVILLGNGTLIILTLLDARLHTPMY
FFLGNLSFLDIWYTSSSIPSMLIHFPSEKKTISFTRCVIQMSVSYTMGST
KCVLLAVMAYDRYVAICNPLRYPIIMGKALCIQMVAVSWGLGFLNSLTET
VLAIRLPFCGKNVINHFVCEILAFVKLACTDTSLNEIIIMLGNVIFLFSP
LLLICISYIFILSTVLRINSAEGRKKAFSTCSAHMTVVIVFYGTILFMYM
KAKSKDSAFDKLIALFYGIVTPMPNPIIYSLRNTEVHGAMRKLMSRPWFW
RK

TABLE G3
DNA Sense Strand Sequence, CG56230-01

CG56230-01: Olfactory Receptor Isoform 1
Acc. No.: GMAC072059_H
>CG56230-01 911 nt

(SEQ ID NO:566)

TAGTAAAGGAGGTCTTGGTCCCAGATGAGTAATTAAAGGAGTATCGAGGTACGTGGAGACATAAGGAGTCCGACATCTAC
TATCCTAACCCGTACCCCCACTGATACGGTATCTTGTCCCGTTAGTCAAACAGTTTTCGTCTCAGAAACCTGAAACGGAA
GTACATGTACTTCTCCTAACAGGGTATTTTGTGTTAGTGGTGACAGTACACCCGACTCGTCCACCTTTTCCGGAAAAAGG
AAGGAAGTCGACTTAACTAAGAATCATGTCATCTTTCCTATTTCTACATCCTCTATGTTTAGTCGTCATTACCTCTTTTG
TTTTTATAATGTAACGGGTTGTAATATTATTAGAGTAAGTTCCTTCATAGACACGTTCGGTCGAACTGTTTCCGGTTATA
AAGTGTTTGTTTTACTAACTACTGTAAAAAAGGTGTCTTCCCATTGGCATAACGTTCTTGTCAAAGACAGTTACTCAACT
CTTTCGGATCAGGGGTTCTCTGTCGGTGGTAGACTTATGTTTCACGGAACGGGTACTACTACCCTATAGAGTCTCCCAAC
GTCTACCGATGTATTGCCAGTATACGGTAGTGACGATCTTCGTGTGTGAACCACCTAGGGTATCACATTCTCTGTCTGTA
AACTTAGTGTGTAGATCACTTCCTTTACCAAAAGAAGAGACTACCCTTCACATAGTCGTAACTCCCCTACCTCCTCCTAC
ACATGGTTTACAGATCCTTCCTCTCTAAGGGTTCCTTCTTCATGTACCCACACACCTCTGCTCGTAGGTCCTCACAGTCT
TACTAGTTCCACGGTAAGGGATCCTCTTAGTGGTCCATGTAGTGATTCGTGTAGTGCTTTTCCTTAAAAAGTCGAGAACC
CATGGGACTTTCAGGAACGTCTTACTTGAGA

RTQ-PCR Human Expression Profiles: Quantitative Expression Analysis of Clones in Various Cells and Tissues

Expression analysis was performed as described in Example C.

CG56230-01: GPCR Olfactory Receptor, Isoform 1

Expression of gene CG56230-01 was assessed using the primer-probe set Ag1652, described in Table G4 and. Results of the RTQ-PCR runs are shown in Tables G5, G6 and G7.

TABLE G4

Probe Name Ag1652

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-CCTCAATGCTGATACACTTCCT-3′ | 58.3 | 22 | 250 | 567 |
| Probe | FAM-5′-CCATCTCCTT CACTAGATGT | 65.6 | 28 | 286 | 568 |
| | GTGATTCA-3′-TAMRA | | | | |
| Reverse | 5′-CGGTGGATCCCATAGTGTAAG-3′ | 59.3 | 21 | 325 | 569 |

TABLE G5
CG56230-01 Panel 1.3D

| | Rel. |
|---|---|
| | Expr., % |
| Tissue Name | 1.3dx4tm5398_ag1652_a1 |
| Adipose | 0 |
| Adrenal gland | 0 |
| Bladder | 0 |
| Bone marrow | 0 |
| Brain (amygdala) | 0 |
| Brain (cerebellum) | 0 |
| Brain (fetal) | 0 |
| Brain (hippocampus) | 0 |
| Cerebral Cortex | 0 |
| Brain (substantia nigra) | 0 |
| Brain (thalamus) | 0 |
| Brain (whole) | 0 |
| Colorectal | 0 |
| Heart (fetal) | 0 |
| Liver adenocarcinoma | 0 |
| Heart | 0 |
| Kidney | 0 |
| Kidney (fetal) | 0 |
| Liver | 0 |
| Liver (fetal) | 0 |
| Lung | 0 |
| Lung (fetal) | 0 |
| Lymph node | 0 |
| Mammary gland | 0 |
| Fetal Skeletal | 0 |
| Ovary | 0 |
| Pancreas | 0 |
| Pituitary gland | 0 |
| Placenta | 0 |
| Prostate | 0 |
| Salivary gland | 0 |
| Skeletal muscle | 0 |
| Small intestine | 0 |
| Spinal cord | 0 |
| Spleen | 0 |
| Stomach | 0 |
| Testis | 0 |
| Thymus | 0 |
| Thyroid | 0 |
| Trachea | 0 |
| Uterus | 0 |
| genomic DNA control | 64.8 |
| Chemistry Control | 100 |

TABLE G6
CG56230-01 Panel 2.2

| | Rel. |
|---|---|
| | Expr., % |
| Tissue Name | 2.2x4tm6360f_ag1652_b1 |
| Normal Colon GENPAK 061003 | 0 |
| 97759 Colon cancer (OD06064) | 0 |
| 97760 Colon cancer NAT (OD06064) | 0 |
| 97778 Colon cancer (OD06159) | 0 |
| 97779 Colon cancer NAT (OD06159) | 0 |
| 98861 Colon cancer (OD06297-04) | 0 |
| 98862 Colon cancer NAT (OD06297-015) | 0 |
| 83237 CC Gr.2 ascend colon (ODO3921) | 0 |
| 83238 CC NAT (ODO3921) | 0 |
| 97766 Colon cancer metastasis (OD06104) | 0 |
| 97767 Lung NAT (OD06104) | 0 |
| 87472 Colon mets to lung (OD04451-01) | 0 |
| 87473 Lung NAT (OD04451-02) | 0 |
| Normal Prostate Clontech A+ 6546-1 | 0 |
| (8090438) | |
| 84140 Prostate Cancer (OD04410) | 0 |
| 84141 Prostate NAT (OD04410) | 0 |
| Normal Ovary Res. Gen. | 0 |
| 98863 Ovarian cancer (OD06283-03) | 0 |
| 98865 Ovarian cancer NAT/fallopian tube | 0 |
| (OD06283-07) | |
| Ovarian Cancer GENPAK 064008 | 83.9 |
| 97773 Ovarian cancer (OD06145) | 0 |
| 97775 Ovarian cancer NAT (OD06145) | 0 |
| 98853 Ovarian cancer (OD06455-03) | 0 |
| 98854 Ovarian NAT (OD06455-07) | 0 |
| Fallopian tube | |
| Normal Lung GENPAK 061010 | 0 |
| 92337 Invasive poor diff. lung adeno | 0 |
| (ODO4945-01 | |
| 92338 Lung NAT (ODO4945-03) | 0 |
| 84136 Lung Malignant Cancer (OD03126) | 0 |
| 84137 Lung NAT (OD03126) | 0 |
| 90372 Lung Cancer (OD05014A) | 0 |
| 90373 Lung NAT (OD05014B) | 0 |
| 97761 Lung cancer (OD06081) | 0 |
| 97762 Lung cancer NAT (OD06081) | 0 |
| 85950 Lung Cancer (OD04237-01) | 0 |
| 85970 Lung NAT (OD04237-02) | 0 |
| 83255 Ocular Mel Met to Liver (ODO4310) | 0 |
| 83256 Liver NAT (ODO4310) | 37.8 |
| 84139 Melanoma Mets to Lung (OD04321) | 0 |
| 84138 Lung NAT (OD04321) | 0 |
| Normal Kidney GENPAK 061008 | 0 |
| 83786 Kidney Ca, Nuclear grade 2 | 0 |
| (OD04338) | |
| 83787 Kidney NAT (OD04338) | 0 |
| 83788 Kidney Ca Nuclear grade 1/2 | 0 |
| (OD04339) | |
| 83789 Kidney NAT (OD04339) | 0 |
| 83790 Kidne Ca, Clear cell type (OD04340) | 0 |
| 83791 Kidney NAT (OD04340) | 50.4 |
| 83792 Kidney Ca, Nuclear grade 3 | 0 |
| (OD04348) | |
| 83793 Kidney NAT (OD04348) | 0 |
| 98938 Kidney malignant cancer | 45.8 |
| (OD06204B) | |
| 98939 Kidney normal adjacent tissue | 0 |
| (OD06204E) | |
| 85973 Kidney Cancer (OD04450-01) | 0 |
| 85974 Kidney NAT (OD04450-03) | 33.8 |
| Kidney Cancer Clontech 8120613 | 0 |
| Kidney NAT Clontech 8120614 | 0 |
| Kidney Cancer Clontech 9010320 | 0 |
| Kidney NAT Clontech 9010321 | 0 |
| Kidney Cancer Clontech 8120607 | 0 |
| Kidney NAT Clontech 8120608 | 0 |
| Normal Uterus GENPAK 061018 | 0 |
| Uterus Cancer GENPAK 064011 | 0 |
| Normal Thyroid Clontech A+ 6570-1 | 0 |
| (7080817) | |
| Thyroid Cancer GENPAK 064010 | 0 |
| Thyroid Cancer INVITROGEN A302152 | 33.2 |
| Thyroid NAT INVITROGEN A302153 | 100 |
| Normal Breast GENPAK 061019 | 0 |
| 84877 Breast Cancer (OD04566) | 0 |
| Breast Cancer Res. Gen. 1024 | 0 |
| 85975 Breast Cancer (OD04590-01) | 0 |
| 85976 Breast Cancer Mets (OD04590-03) | 0 |
| 87070 Breast Cancer Metastasis | 52.6 |
| (OD04655-05) | |
| GENPAK Breast Cancer 064006 | 0 |
| Breast Cancer Clontech 9100266 | 0 |
| Breast NAT Clontech 9100265 | 0 |
| Breast Cancer INVITROGEN A209073 | 0 |
| Breast NAT INVITROGEN A2090734 | 0 |
| 97763 Breast cancer (OD06083) | 0 |
| 97764 Breast cancer node metastasis | 0 |
| (OD06083) | |
| Normal Liver GENPAK 061009 | 45.2 |
| Liver Cancer Research Genetics RNA 1026 | 0 |
| Liver Cancer Research Genetics RNA 1025 | 67.3 |
| Paired Liver Cancer Tissue Research | 0 |
| Genetics RNA 6004-T | |
| Paired Liver Tissue Research Genetics | 0 |
| RNA 6004-N | |
| Paired Liver Cancer Tissue Research | 0 |
| Genetics RNA 6005-T | |
| Paired Liver Tissue Research Genetics | 0 |
| RNA 6005-N | |
| Liver Cancer GENPAK 064003 | 0 |
| Normal Bladder GENPAK 061001 | 0 |
| Bladder Cancer Research Genetics RNA | 0 |
| 1023 | |
| Bladder Cancer INVITROGEN A302173 | 0 |
| Normal Stomach GENPAK 061017 | 0 |
| Gastric Cancer Clontech 9060397 | 0 |
| NAT Stomach Clontech 9060396 | 0 |
| Gastric Cancer Clontech 9060395 | 0 |
| NAT Stomach Clontech 9060394 | 0 |
| Gastric Cancer GENPAK 064005 | 0 |

TABLE G7
CG56230-01 Panel 4D

| | Rel. Expr., % |
|---|---|
| | 4dx4tm5100f_{—} |
| Tissue Name | ag1652_a2 |
| 93768_Secondary Th1_anti-CD28/anti-CD3 | 0 |
| 93769_Secondary Th2_anti-CD28/anti-CD3 | 0 |
| 93770_Secondary Tr1_anti-CD28/anti-CD3 | 0 |
| 93573_Secondary Th1_resting day 4-6 in IL-2 | 0 |
| 93572_Secondary Th2_resting day 4-6 in IL-2 | 0 |
| 93571_Secondary Tr1_resting day 4-6 in IL-2 | 0 |
| 93568_primary Th1_anti-CD28/anti-CD3 | 0 |
| 93569_primary Th2_anti-CD28/anti-CD3 | 0 |
| 93570_primary Tr1_anti-CD28/anti-CD3 | 0 |
| 93565_primary Th1_resting dy 4-6 in IL-2 | 0 |
| 93566_primary Th2_resting dy 4-6 in IL-2 | 0 |
| 93567_primary Tr1_resting dy 4-6 in IL-2 | 50.7 |
| 93351_CD45RA CD4 lymphocyte_anti-CD28/anti-CD3 | 0 |
| 93352_CD45RO CD4 lymphocyte_anti-CD28/anti-CD3 | 0 |
| 93251_CD8 Lymphocytes_anti-CD28/anti-CD3 | 0 |
| 93353_chronic CD8 Lymphocytes 2ry_resting dy | 0 |
| 4-6 in IL-2 | |
| 93574_chronic CD8 Lymphocytes 2ry_activated | 18 |
| CD3/CD28 | |
| 93354_CD4_none | 0 |
| 93252_Secondary Th1/Th2/Tr1_anti-CD95 CH11 | 0 |
| 93103_LAK cells_resting | 0 |
| 93788_LAK cells_IL-2 | 0 |
| 93787_LAK cells_IL-2 + IL-12 | 0 |
| 93789_LAK cells_IL-2 + IFN gamma | 14.5 |
| 93790_LAK cells_IL-2 + IL-18 | 0 |
| 93104_LAK cells_PMA/ionomycin and IL-18 | 0 |
| 93578_NK Cells IL-2_resting | 0 |
| 93109_Mixed Lymphocyte Reaction_Two Way MLR | 0 |
| 93110_Mixed Lymphocyte Reaction_Two Way MLR | 0 |
| 93111_Mixed Lymphocyte Reaction_Two Way MLR | 0 |
| 93112_Mononuclear Cells (PBMCs)_resting | 0 |
| 93113_Mononuclear Cells (PBMCs)_PWM | 30.9 |
| 93114_Mononuclear Cells (PBMCs)_PHA-L | 0 |
| 93249_Ramos (B cell)_none | 0 |
| 93250_Ramos (B cell)_ionomycin | 0 |
| 93349_B lymphocytes_PWM | 0 |
| 93350_B lymphoytes_CD40L and IL-4 | 0 |
| 92665_EOL-1 (Eosinophil)_dbcAMP differentiated | 0 |
| 93248_EOL-1 (Eosinophil)_dbcAMP/PMAionomycin | 0 |
| 93356_Dendritic Cells_none | 0 |
| 93355_Dendritic Cells_LPS 100 ng/ml | 0 |
| 93775_Dendritic Cells_anti-CD40 | 0 |
| 93774_Monocytes_resting | 0 |
| 93776_Monocytes_LPS 50 ng/ml | 0 |
| 93581_Macrophages_resting | 0 |
| 93582_Macrophages_LPS 100 ng/ml | 0 |
| 93098_HUVEC (Endothelial)_none | 0 |
| 93099_HUVEC (Endothelial)_starved | 0 |
| 93100_HUVEC (Endothelial)_IL-1b | 0 |
| 93779_HUVEC (Endothelial)_IFN gamma | 0 |
| 93102_HUVEC (Endothelial)_TNF alpha + IFN gamma | 0 |
| 93101_HUVEC (Endothelial)_TNF alpha + IL4 | 0 |
| 93781_HUVEC (Endothelial)_IL-11 | 0 |
| 93583_Lung Microvascular Endothelial Cells_none | 0 |
| 93584_Lung Microvascular Endothelial Cells_{—} | 0 |
| TNF a (4 ng/ml) and IL1b (1 ng/ml) | |
| 92662_Microvascular Dermal endothelium_none | 0 |
| 92663_Microsvasular Dermal endothelium_{—} | 0 |
| TNF a (4 ng/ml) and IL1b (1 ng/ml) | |
| 93773_Bronchial epithelium_TNF a (4 ng/ml) | 0 |
| and IL1b (1 ng/ml)** | |
| 93347_Small Airway Epithelium_none | 0 |
| 93348_Small Airway Epithelium_{—} | 0 |
| TNF a (4 ng/ml) and IL1b (1 ng/ml) | |
| 92668_Coronery Artery SMC_resting | 0 |
| 92669_Coronery Artery SMC_TNF a | 0 |
| (4 ng/ml) and IL1b (1 ng/ml) | |
| 93107_astrocytes_resting | 0 |
| 93108_astrocytes_TNF a (4 ng/ml) and IL1b (1 ng/ml) | 0 |
| 92666_KU-812 (Basophil)_resting | 0 |
| 92667_KU-812 (Basophil)_PMA/ionoycin | 0 |
| 93579_CCD1106 (Keratinocytes)_none | 0 |
| 93580_CCD1106 (Keratinocytes)_TNF a and IFNg** | 0 |
| 93791_Liver Cirrhosis | 34.2 |
| 93792_Lupus Kidney | 0 |
| 93577_NCI-H292 | 0 |
| 93358_NCI-H292_IL-4 | 0 |
| 93360_NCI-H292_IL-9 | 0 |
| 93359_NCI-H292_IL-13 | 0 |
| 93357_NCI-H292_IFN gamma | 0 |
| 93777_HPAEC_- | 0 |
| 93778_HPAEC_IL-1 beta/TNA alpha | 0 |
| 93254_Normal Human Lung Fibroblast_none | 0 |
| 93253_Normal Human Lung Fibroblast_{—} | 0 |
| TNF a (4 ng/ml) and IL-1b (1 ng/ml) | |
| 93257_Normal Human Lung Fibroblast_IL-4 | 0 |
| 93256_Normal Human Lung Fibroblast_IL-9 | 21.8 |
| 93255_Normal Human Lung Fibroblast_IL-13 | 0 |
| 93258_Normal Human Lung Fibroblast_IFN gamma | 0 |
| 93106_Dermal Fibroblasts CCD1070_resting | 0 |
| 93361_Dermal Fibroblasts CCD1070_TNF alpha | 0 |
| 4 ng/ml | |
| 93105_Dermal Fibroblasts CCD1070_IL-1 beta 1 ng/ml | 0 |
| 93772_dermal fibroblast_IFN gamma | 0 |
| 93771_dermal fibroblast_IL-4 | 0 |
| 93259_IBD Colitis 1** | 0 |
| 93260_IBD Colitis 2 | 0 |
| 93261_IBD Crohns | 0 |
| 735010_Colon_normal | 0 |
| 735019_Lung_none | 0 |
| 64028-1_Thymus_none | 0 |
| 64030-1_Kidney_none | 100 |

H. NOV27b—Human Carboxypeptidase A2-Like Protein—CG56246-02

Rationale for Use as a Diagnostic and/or Target for Small Molecule Drugs and Antibody Therapeutics.

Endocrine balance within the body is maintained by a variety of peptides and peptide hormones, such as insulin, glucagon-like peptide, proopiomelanocortin etc. Several of these agents are subject to activation by proteolytic cleavage and the expression or non-expression of relevant proteases can be expected to have dramatic effects on pathophysiology. For example, carboxypeptidase E-deficient mice show deficiencies in hormone maturation, leading to obesity with mild diabetes (Friis-Hansen et a;., J Endocrinol June 2001;169(3):595-602). Therefore, there is precedence for the role of proteases in both obesity and diabetes. In the GeneCalling® studies described, the upregulation of the carboxypeptidase A2 in the liver of the spontaneous hypertensive rat, which can be abolished by thiazolidinedione treatment, suggest that insulin insensitivity in this animal model may be coupled to increased proteolysis. Therefore, the inhibition of CPA2 may be an effective way to reduce insulin resistance in the liver.

Besides for a role in insulin sensitivity, our GeneCalling and Pathcalling data show that CPA2 may be involved in satiety. Firstly, GeneCalling indicate that in the duodenum of fasted and subsequently refed rats this gene is 45 fold downregulated when compared with fasted rats. This indicated that CPA2 expression is linked to hunger signals initiated in the gut, which are high in the fasted state and low in the refed state. Pathcalling confirm the influence of CPA2 on satiety mechanisms, by showing that CPA2 interacts with cholecystokinin, a gut hormone which has been clearly suggested to be a physiological satiety factor. Our data show that most likely CPA2 is involved in the degradation of CCK, and thereby induces hunger. The downregulation of CPA2 therefore may be an effective therapeutic for obesity since it may decrease hunger.

Rat Dietary-Induced Obesity Fast-Re-feed Study (BP24.06)

This study was designed to examine the chronic gene expression changes in response to dietary-induced obesity (DIO), as well as the acute gene expression changes associated with fasting and re-feeding. The sample groups for the study were selected from male Wistar rats and were either chow-fed, or placed on a high fat (45%) diet. The rats on the high-fat diet were further sub-divided into rats resistant to DIO (<1 standard deviation above the weight of chow-fed control rats) and DIO rats (4 standard deviations above the weight of chow-fed control rats. Changes in gene expression in the three sample groups were examined under normal feeding conditions, after 24 hr fasting, and after 24 hr fasting followed by a 4-hr re-feeding period. The clinical data obtained from each animal included body weight, food intake, glucose levels, insulin levels, free fatty acid levels and blood chemistry. A variety of tissues were harvested, including hypothalamus, brainstem, striatum, epididymal fat pads, subcutaneous fat pad, brown adipose tissue (BAT), gastrocnemius muscle (fast twitch skeletal muscle), soleus muscle (slow twitch skeletal muscle,), proximal small intestine, distal small intestine, pituitary, kidneys, adrenal gland, and heart. The differential gene expression profiles for these tissues should reveal genes and pathways that can be used as therapeutic targets for obesity.

Species #3 Rat Strains: Wistar

Results of Rat Dietary-Induced Obesity Fast-Re-feed Study (BP24.06)

A gene fragment of the rat Carboxypeptidase A2 was also found to be downregulated by 45 fold in the duodenum of fasted and refed rats when compared to rats that were fasted using CuraGen's GeneCalling® method of differential gene expression. A differentially expressed rat gene fragment migrating, at approximately 290.8 nucleotides in length (Table H1a—vertical line) was definitively identified as a component of the rat Carboxypeptidase A2 cDNA. The method of direct sequencing was used for confirmation of gene assessment and revealed that this fragment belonged to the rat carboxypeptidase A2 gene. Competitive PCR was then performed using this sequence to ablate the peak (grey trace).

TABLE H2

The direct sequence of the 267.1 nucleotide-long gene
fragment is shown below.

(SEQ ID NO:570)

GATCTGCTTG GCTGGCAGGA GGAAGCCATA GAAACCTGTG TCCCTCAGTT CAAAGGCAAA TGAGTATTTG
ATGCCAAGGT CGTAAGCCCA GTCGATGCTT CCACCACTCG CCTGGTAGAT GACAGAACAG ATGGGTCCCA
CTTTATAACT GGTGCCGTGC AGTCTTTTCA AAGCCTGGGC AGCCTTCTGG GCCACTTCAT CCAGCTCATT
AAAGTCATCT GGCTTGGTAC ATTTATAGCC ATAGGGGAAC ATAAGCAGTT GGGAATAGCT GTGAAGGGTA
ATAAAAGCT

Gene-specific primers were designed to the above sequence and used for competitive PCR.

[image]

This differentially expressed gene fragment in Discovery Study BP24.06 is from the rat Carboxypeptidase A2 gene.

Findings: The following illustration suggests how alterations in expression of the human CPA2 and associated gene products function in the etiology and pathogenesis of obesity and/or diabetes (Table H4). The scheme shows the unique findings of these discovery studies in conjunction with what has been reported in the literature. The outcome of inhibiting the action of the human CPA2 would lead to a reduction in food intake.

[image]

PathCalling screening identified the interaction between Carboxypeptidase A2 and cholecystokinin (CCK). Cholecystokinin is a gut hormone and a neuropeptide that has the capacity to stimulate insulin secretion. Administration of CCK has been proposed as a potential treatment for type II diabetes. Results from PathCalling suggest that Carboxypeptidase A2 may be involved in degradation of CCK. Thus, an antagonist of Carboxypeptidase A2 may be beneficial for stimulation of insulin secretion in type II Diabetes.

In Frame Cloning: In frame cloning is a process designed to insert DNA sequences into expression vectors such that the encoded proteins can be produced. The expressed proteins were either full length or corresponding to specific domains of interest. The PCR template was based on a previously identified plasmid (the PCR product derived by exon linking, covering the entire open reading frame) when available, or on human cDNA(s). The human cDNA pool was composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus. For downstream cloning purposes, the forward and reverse primers included in-frame EcoRI and NotI restriction sites. The amplified product was detected by agarose gel electrophoresis. The fragment was gel-purified and ligated into the pcDNA3.1+, and pFastBac1 (Invitrogen, Carlsbad, Calif.) following the manufacturer's recommendation. Twenty four clones per transformation were picked and a quality control step was performed to verify that these clones contain an insert of the anticipated size. Subsequently, eight of these clones were sequenced, and assembled in a fashion similar to the SeqCalling process. In addition to analysis of the entire sequence assembly, sequence traces were evaluated manually.

Findings: Table H5 depicts the preferred cDNA(s), among the variants listed above, that encompass the coding portion of the human CPA2 for expression of recombinant protein from any number of plasmid, phage or phagemid vectors in a variety of cellular systems for screening purposes. The corresponding amino acid sequence(s) is also listed. Although the sequences below are the preferred isoforms, any of the other isoforms may be used for similar purposes. Furthermore, under varying assay conditions, conditions may dictate that another isoform may supplant the listed isoforms. As shown in Table H2B, the open reading frame of the working representatives of CG56246-02 have 1 aa difference when compared to CG56246-04. CG56246-04 contains an N-terminal histidine tag and CG56246-05 contains a C-terminal Histidine tag used for protein purification (not visible in ClustalW).

Tables H5a-H5f disclose physical cDNA clones available for expression and screening purposes.

TABLE H5a
CG56246-03

>CG56246-03, 1258 nt

(SEQ ID NO:571)

TAATAGGGGTGGTCTCGCACATGCTCCATGATTGCCTTCAAGCCAAGCCAGGTCTCCTCGGCTGTGGGCAGGATCTGACG
GGCTGGCAAGAGGAAGCCGTAGCGCCCTGTGTCTCTCAGTTCAAAGGCAAATGAGTACTTGATGCCATAATCATAGGACC
AGTCAATGCTTCCTCCACTGGCTTGGTAGATGACAGAGCAGATTGGTCCCACTTTGTACTTGGTGCCATGCAGGCTTCTC
AGAGATTGGGCAGCCTTTTGGGCCACTTCACTCAGCTCATCAAAGTCATCTAACTTGGTACATTTGTACCCATAGGGGAA
CATCAGCAGCTGGGAATAGCTGTGGAGGGTAATGAAGGCCTTGACTTTTCCATGACTCTTGATGAAGTCCACTATGGATT
TCACTTCAACTTCAGAGTTGGCACTGGGTCCGTGGTATGAATCAGAGCAAGGGTTGCTGCTGGCTCCAGGTCCTCCAAAA
CCTGCATCCCAGTTCCGGTTAGGATCCACACCAACACAGAGGCTTCCAGATACCTTGGACCGGGTCTTCCGCCACATACG
ATTTTTGGTTTGAGAGAACACGTATCCATCAGGGTTTGTGACTGGCAGGAGGAAGATATCCAGGGCGTCCAGAATGGAAG
TGATGGATGGGTCCTTTCCATAATCAGAAACAATCTTATTTGCTGTCCAAAGTGCCGTAGCTTGTGTAACCCACTCTCGA
GCATGGATCCCAGCATCCAGCCAGATAGCTGGCTTGTCTCCTCCGGTGCTGAACTTGAGCACGTTCATAGGCCGGTTCTC
AAAAGAAGAGCCAATATTCACTTTGCTCACTAGACCAGGGTGCTCAGCCACGAGGTTATCCATTTCTTGGGAAATCTCTT
CCAGGGTATGGTAGGCCCCAAAATTGAAGTTACCACTCCGTTCTCTTCTCCTATTAAAAAGCATTTCTTCATTCTCTTTG
TCCAACAGGACCTGCACGTCTTCAATCATGATGGAATAGGCAATTCCCTGGGACTCCAAGAACACTTTGACTGCCTGGAC
GTTGACGAAGGGAACTCGGACGTGGGCTGTCTCCCCTGGGGTGGTGGGTGATTTCCAAAAATCAAGCTGGAGATGTTCTT
GAGCCTCCAATTGTAGCAGATTTTTAATTTGTTCTTCATTGCTTGGTACAATCTCAAGAACTTGGTCTCCCACAAATGTT
TCTAGACAGTAGATATGCCCAAAAAGGGCACCAAAAAACAGGATCAACCTCATGGTGG

TABLE H5b
CG56246-03

>CG56246-03-prot 418 aa

(SEQ ID NO:572)

TMRLILFFGALFGHIYCLETFVGDQVLEIVPSNEEQIKNLLQLEAQEHLQLDFWKSPTTPGETAHVRVPFVNVQAVKVFL
ESQGIAYSIMIEDVQVLLDKENEEMLFNRRRERSGNFNFGAYHTLEEISQEMDNLVAEHPGLVSKVNIGSSFENRPMNVL
KFSTGGDKPAIWLDAGIHAREWVTQATALWTANKIVSDYGKDPSITSILDALDIFLLPVTNPDGYVFSQTKNRMWRKTRS
KVSGSLCVGVDPNRNWDAGFGGPGASSNPCSDSYHGPSANSEVEVKSIVDFIKSHGKVKAFITLHSYSQLLMFPYGYKCT
KLDDFDELSEVAQKAAQSLRSLHGTKYKVGPICSVIYQASGGSIDWSYDYGIKYSFAFELRDTGRYGFLLPARQILPTAE
ETWLGLKAIMEHVRDHPY

TABLE H5c
CG56246-04

>CG56246-04, 1279 nt

(SEQ ID NO:573)

CCACCATGGGCCACCATCACCACCATCACAGGTTGATCCTGTTTTTTGGTGCCCTTTTTGGGCATATCTACTGTCTAGAA
ACATTTGTGGGAGACCAAGTTCTTGAGATTGTACCAAGCAATGAAGAACAAATTAAAAATCTGCTACAATTGGAGGCTCA
AGAACATCTCCAGCTTGATTTTTGGAAATCACCCACCACCCCAGGGGAGACAGCCCACGTCCGAGTTCCCTTCGTCAACG
TCCAGGCAGTCAAAGTGTTCTTGGAGTCCCAGGGAATTGCCTATTCCATCATGATTGAAGACGTGCAGGTCCTGTTGGAC
AAAGAGAATGAAGAAATGCTTTTTAATAGGAGAAGAGAACGGAGTGGTAACTTCAATTTTGGGGCCTACCATACCCTGGA
AGAGATTTCCCAAGAAATGGATAACCTCGTGGCTGAGCACCCTGGTCTAGTGAGCAAAGTGAATATTGGCTCTTCTTTTG
AGAACCGGCCTATGAACGTGCTCAAGTTCAGCACCGGAGGAGACAAGCCAGCTATCTGGCTGGATGCTGGGATCCATGCT
CGAGAGTGGGTTACACAAGCTACGGCACTTTGGACAGCAAATAAGATTGTTTCTGATTATGGAAAGGACCCATCCATCAC
TTCCATTCTGGACGCCCTGGATATCTTCCTCCTGCCAGTCACAAACCCTGATGGATACGTGTTCTCTCAAACCAAAAATC
GTATGTGGCGGAAGACCCGGTCCAAGGTATCTGGAAGCCTCTGTGTTGGTGTGGATCCTAACCGGAACTGGGATGCAGGT
TTTGGAGGACCTGGAGCCAGCAGCAACCCTTGCTCTGATTCATACCACGGACCCAGTGCCAACTCTGAAGTTGAAGTGAA
ATCCATAGTGGACTTCATCAAGAGTCATGGAAAAGTCAAGGCCTTCATTACCCTCCACAGCTATTCCCAGCTGCTGATGT
TCCCCTATGGGTACAAATGTACCAAGTTAGATGACTTTGATGAGCTGAGTGAAGTGGCCCAAAAGGCTGCCCAATCTCTG
AGAAGCCTGCATGGCACCAAGTACAAAGTGGGACCAATCTGCTCTGTCATCTACCAAGCCAGTGGAGGAAGCATTGACTG
GTCCTATGATTATGGCATCAAGTACTCATTTGCCTTTGAACTGAGAGACACAGGGCGCTACGGCTTCCTCTTGCCAGCCC
GTCAGATCCTGCCCACAGCCGAGGAGACCTGGCTTGGCTTGAAGGCAATCATGGAGCATGTGCGAGACCACCCCTATTA

TABLE H5d
CG56246-04

>CG56246-04-prot 425 aa

(SEQ ID NO:574)

TMGHHHHHHRLILFFGALFGHIYCLETFVGDQVLEIVPSNEEQIKNLLQLEAQEHLQLDFWKSPTTPGETAHVRVPFVNV
QAVKVFLESQGIAYSIMIEDVQVLLDKENEEMLFNRRRERSGNFNFGAYHTLEEISQEMDNLVAEHPGLVSKVNIGSSFE
NRPMNVLKFSTGGDKPAIWLDAGIHAREWVTQATALWTANKIVSDYGKDPSITSILDALDIFLLPVTNPDGYVFSQTKNR
MWRKTRSKVSGSLCVGVDPNRNWDAGFGGPGASSNPCSDSYHGPSANSEVEVKSIVDFIKSHGKVKAFITLHSYSQLLMF
PYGYKCTKLDDFDELSEVAQKAAQSLRSLHGTKYKVGPICSVIYQASGGSIDWSYDYGIKYSFAFELRDTGRYGFLLPAR
QILPTAEETWLGLKAIMEHVRDHPY

TABLE H5e
CG56246-05

>CG56246-05, 1276 nt

(SEQ ID NO:575)

CCACCATGAGGTTGATCCTGTTTTTTGGTGCCCTTTTTGGGCATATCTACTGTCTAGAAACATTTGTGGGAGACCAAGTT
CTTGAGATTGTACCAAGCAATGAAGAACAAATTAAAAATCTGCTACAATTGGAGGCTCAAGAACATCTCCAGCTTGATTT
TTGGAAATCACCCACCACCCCAGGGGAGACAGCCCACGTCCGAGTTCCCTTCGTCAACGTCCAGGCAGTCAAAGTGTTCT
TGGAGTCCCAGGGAATTGCCTATTCCATCATGATTGAAGACGTGCAGGTCCTGTTGGACAAAGAGAATGAAGAAATGCTT
TTTAATAGGAGAAGAGAACGGAGTGGTAACTTCAATTTTGGGGCCTACCATACCCTGGAAGAGATTTCCCAAGAAATGGA
TAACCTCGTGGCTGAGCACCCTGGTCTAGTGAGCAAAGTGAATATTGGCTCTTCTTTTGAGAACCGGCCTATGAACGTGC
TCAAGTTCAGCACCGGAGGAGACAAGCCAGCTATCTGGCTGGATGCTGGGATCCATGCTCGAGAGTGGGTTACACAAGCT
ACGGCACTTTGGACAGCAAATAAGATTGTTTCTGATTATGGAAAGGACCCATCCATCACTTCCATTCTGGACGCCCTGGA
TATCTTCCTCCTGCCAGTCACAAACCCTGATGGATACGTGTTCTCTCAAACCAAAAATCGTATGTGGCGGAAGACCCGGT
CCAAGGTATCTGGAAGCCTCTGTGTTGGTGTGGATCCTAACCGGAACTGGGATGCAGGTTTTGGAGGACCTGGAGCCAGC
AGCAACCCTTGCTCTGATTCATACCACGGACCCAGTGCCAACTCTGAAGTTGAAGTGAAATCCATAGTGGACTTCATCAA
GAGTCATGGAAAAGTCAAGGCCTTCATTACCCTCCACAGCTATTCCCAGCTGCTGATGTTCCCCTATGGGTACAAATGTA
CCAAGTTAGATGACTTTGATGAGCTGAGTGAAGTGGCCCAAAAGGCTGCCCAATCTCTGAGAAGCCTGCATGGCACCAAG
TACAAAGTGGGACCAATCTGCTCTGTCATCTACCAAGCCAGTGGAGGAAGCATTGACTGGTCCTATGATTATGGCATCAA
GTACTCATTTGCCTTTGAACTGAGAGACACAGGGCGCTACGGCTTCCTCTTGCCAGCCCGTCAGATCCTGCCCACAGCCG
AGGAGACCTGGCTTGGCTTGAAGGCAATCATGGAGCATGTGCGAGACCACCCCTATCACCATCACCACCATCACTA

TABLE H5f
CG56246-05

>CG56246-05-prot 417 aa

(SEQ ID NO:576)

MRLILFFGALFGHIYCLETFVGDQVLEIVPSNEEQIKNLLQLEAQEHLQLDFWKSPTTPGETAHVRVPFVNVQAVKVFLE
SQGIAYSIMIEDVQVLLDKENEEMLFNRRRERSGNFNFGAYHTLEEISQEMDNLVAEHPGLVSKVNIGSSFENRPMNVLK
FSTGGDKPAIWLDAGIHAREWVTQATALWTANKIVSDYGKDPSITSILDALDIFLLPVTNPDGYVFSQTKNRMWRKTRSK
VSGSLCVGVDPNRNWDAGFGGPGASSNPCSDSYHGPSANSEVEVKSIVDFIKSHGKVKAFITLHSYSQLLMFPYGYKCTK
LDDFDELSEVAQKAAQSLRSLHGTKYKVGPICSVIYQASGGSIDWSYDYGIKYSFAFELRDTGRYGFLLPARQILPTAEE
TWLGLKAIMEHVRDNPY

[image]

I. NOV28b—Human SERCA1-Like Protein—CG57417-03

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by CuraGen Corporation in certain cases. The SERCA1 adult isoform-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

Discovery Process: The following sections describe the study design(s) and the techniques used to identify the SERCA1 adult isoform-encoded protein and any variants, thereof, as being suitable as diagnostic markers, targets for an antibody therapeutic and targets for a small molecule drugs for Obesity and Diabetes.

Studies: MB.04 Obese versus Lean Mice (Genetic)

Study Statements: MB04. Large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

Species #1 MOUSE Strains NZB, SM/J, C57B1/6, Cast/Ei

SERCA1 Adult Isoform:

The SERCA1 adult isoform is a magnesium dependent enzyme that catalyzes the hydrolysis of ATP coupled with the translocation of calcium from the cytosol to the sarcoplasmic reticulum lumen. It contributes to calcium sequestration involved in muscular excitation/contraction. SERCA 1 is an integral membrane protein of the sarcoplasmic and endoplasmic reticulum and has 2 alternative spliced isoforms, serca1a/atp2a1a/adult and serca1b/atp2a1b/neonatal. The SERCA1 adult isoform accounts for more than 99% of serca1 isoforms expressed in adult, while isoform serca1b predominates in neo-natal fibers. Defects in atp2a1 are associated with some forms of the autosomal recessive inheritance of the brody disease (bd), characterized by increasing impairment of relaxation of fast twist skeletal muscle during exercise.

SPECIES #1 mouse (NZB vs SM/J) FIGS. 1A and 1B show that a gene fragment of the mouse SERCA1 was initially found to be down-regulated by 16.4 fold in the adipose tissue of NZB mice relative to SM/J mice using CuraGen's GeneCalling™ method of differential gene expression. A differentially expressed mouse gene fragment migrating at approximately 277 nucleotides in length (Tables I1A and I1B—vertical line) was definitively identified as a component of the mouse SERCA1 cDNA in the NZB and SM/J adipose (in the graphs, the abscissa is measured in lengths of nucleotides and the ordinate is measured as signal response). The method of competitive PCR was used for conformation of the gene assessment. The electropherogramatic peaks corresponding to the gene fragment of the mouse SERCA1 are ablated when a gene-specific primer (see below) which competes with primers in the linker-adaptors during the PCR amplification. The peaks at 277 nt in length are ablated in the sample from both the NZB and SM/J mice. The altered expression in of these genes in the animal model support the role of SERCA1 in the pathogenesis of obesity and/or diabetes.

SPECIES #1 mouse (C57B1/6 vs Cast/Ei) The public partial sequence of mouse SERCA1 of 1045 nucleotides was amplified by PCR from the Cast/Ei and C57B1/6 mouse strains and directly sequenced. Comparison of the two obtained sequences of the C57B1/6 and Cast/Ei strain shows a mutation in the form of a deletion of a cytosine in the SERCA1 coding sequence in Cast/Ei leading to a stopcodon in the open reading frame (For alignment, see Table I3). The mutation occurs in between the 6^{th} and the 7^{th} transmembrane region of the ATPase and leads to the ablation of the calcium transporting function of SERCA1 in the Cast/Ei.

TABLE I1
Partial Mouse SERCA1 Gene Sequence

Identified fragment from 372 to 648 in bold. band size: 277

(SEQ ID NO:581)

TTGACTTTTCTGTCATTTATTTTCAATAAATAAGCAATCAGCTAGTCAGTTGCCTTGTGCCTGCAAGCCCCGTGAGTTCG
GGAAGGGGATTTACAAGGTTCGGAGGGAGAGCGGGTTGCTGAAGGGGACGAGGGTGGAGGACTTTATTTATAAACAGAAT
TGAGGGGGAAGAAGGGTCAGTGCCTCAGCTTTGGCTGAAGATGCATGGCTATTGGGGTGGGGAACACAGGGCACAAGGGC
TGGTTACTTCCTTCTCTCGTCTTCTGGATCTGTGACACGGTTCAAAGACATGGAGGAGGGGGGTGGTTATCCCTCCAGAT
AGTTCCGAGCAATGAACTTGAGAAGCTCATCCAGCCCGATGACTGGCAGTGAGATCTTGAGGACCATGAGCCACTGGGTA
AAGTCCAGGGCCCGGAGCTTGAAGATCATCGGCAGGGGGTCGACATAGAGGATGAGGAAGTGGAGGGACATGGACAGGCA
GATGGAACCCAGCAGCCAGATGTTCACCCAGGGTGGCATCCGCAGTAGGGACTGGTTCTCAGACAGGCTGTTGAGAGCAT
TGCACATCTCGATGGTCACCAACACAGACAAGGCCATGGTCATGGGCTCGGGGGCCTCAAAGACCTCACAGTCCAGGCCA
TCGAATTCAGGGTTGTGCTCAGTGCACTGCATGAAATGAGTCAGCTGATGGTAGCTGACATGAGGCCCGTCCTCTGCATA
CAAAAACCACCAGGCAGCTGCTCCTACAGTGGCTGCACCCACATAGCCCCCAATTGCCATGTAGCGGAAAAAGAGCCAGC
CACTGATAAGAGGCTCCTTGGGACTCCTGGGGGGGCGGTCCATGATGTCCAGGTCAGGTGGGTTGAATCCCAGGGCAGTA
GCCGGGAGCCCATCAGTCACCAAGTTCACCCAGAGCAGCTGCACAGGGATCAGAGCCTCAGCGAGCCCCAAGGCTGCTGT
CAAGAAGATACAGACCACCTCGCCCACATTGGAGGAGATGAGGTAGCGGATGAACTGCTTCATGTTGTTGTAGATGGCGC
GGCCC

[image] [image] TABLE I4
Human SERCA1 adult isoform DNA and Protein Sequence CG57417-03

SEQ ID NO:586

| | |
|---|---|
| 1 | ATGGAGGCCGCTCATGCTAAAACCACGGAGGAATGTTTGGCCTATTTTGGGGTGAGTGAGACCACGGGCCTCACCCCGGA |
| 81 | CCAAGTTAAGCGGAATCTGGAGAAATACGGCCTCAATGAGCTCCCTGCTGAGGAAGGGAAGACCCTGTGGGAGCTGGTGA |
| 161 | TAGAGCAGTTTGAAGACCTCCTGGTGCGGATTCTCCTCCTGGCCGCATGCATTTCCTTCGTGCTGGCCTGGTTTGAGGAA |
| 241 | GGTGAAGAGACCATCACTGCCTTTGTTGAACCCTTTGTCATCCTCTTGATCCTCATTGCCAATGCCATCGTGGGGGTTTG |
| 321 | GCAGGAGCGGAACGCAGAGAACGCCATCGAGGCCCTGAAGGAGTATGAGCCAGAGATGGGGAAGGTCTACCGGGCTGACC |
| 401 | GCAAGTCAGTGCAAAGGATCAAGGCTCGGGACATCGTCCCTGGGGACATCGTGGAGGTGGCTGTGGGGGACAAAGTCCCT |
| 481 | GCAGACATCCGAATCCTCGCCATCAAATCCACCACGCTGCGGGTTGACCAGTCCATCCTGACAGGCGAGTCTGTATCTGT |
| 561 | CATCAAACACACGGAGCCCGTTCCTGACCCCCGAGCTGTCAACCAGGACAAGAAGAACATGCTTTTCTCGGGCACCAACA |
| 641 | TTGCAGCCGGCAAGGCCTTGGGCATCGTGGCCACCACCGGTGTGGGCACCGAGATTGGGAAGATCCGAGACCAAATGGCT |
| 721 | GCCACAGAACAGGACAAGACCCCCTTGCAGCAGAAGCTGGATGAGTTTGGGGAGCAGCTCTCCAAGGTCATCTCCCTCAT |
| 801 | CTGTGTGGCTGTCTGGCTTATCAACATTGGCCACTTCAACGACCCCGTCCATGGGGGCTCCTGGTTCCGCGGGGCCATCT |
| 881 | ACTACTTTAAGATTGCCGTGGCCTTGGCTGTGGCTGCCATCCCCGAAGGTCTTCCTGCAGTCATCACCACCTGCCTGGCC |
| 961 | CTGGGTACCCGTCGGATGGCAAAGAAGAATGCCATTGTAAGAAGCTTGCCCTCCGTAGAGACCCTGGGCTGCACCTCTGT |
| 1041 | CATCTGTTCCGACAAGACAGGCACCCTCACCACCAACCAGATGTCTGTCTGCAAGATGTTTATCATTGACAAGGTGGATG |
| 1121 | GGGACATCTGCCTCCTGAATGAGTTCTCCATCACCGGCTCCACTTACGCTCCAGAGGGAGAGGTCTTGAAGAATGATAAG |
| 1201 | CCAGTCCGGCCAGGGCAGTATGACGGGCTGGTGGAGCTGGCCACCATCTGTGCCCTCTGCAATGACTCCTCCTTGGACTT |
| 1201 | CCAGTCCGGCCAGGGCAGTATGACGGGCTGGTGGAGCTGGCCACCATCTGTGCCCTCTGCAATGACTCCTCCTTGGACTT |
| 1361 | TGTTCAACACGGATGTGAGAAGCCTCTCGAAGGTGGAGAGAGCCAACGCCTGCAACTCGGTGATCCGCCAGCTAATGAAG |
| 1441 | AAGGAATTCACCCTGGAGTTCTCCCGAGACAGAAAGTCCATGTCTGTCTATTGCTCCCCAGCCAAATCTTCCCGGGCTGC |
| 1521 | TGTGGGCAACAAGATGTTTGTCAAGGGTGCCCCTGAGGGCGTCATCGACCGCTGTAACTATGTGCGAGTTGGCACCACCC |
| 1601 | GGGTGCCACTGACGGGGCCGGTGAAGGAAAAGATCATGGCGGTGATCAAGGAGTGGGGCACTGGCCGGGACACCCTGCGC |
| 1681 | TGCTTGGCCCTGGCCACCCGGGACACCCCCCCGAAGCGAGAGGAAATGGTCCTGGATGACTCTGCCAGGTTCCTGGAGTA |
| 1761 | TGAGACGGACCTGACATTCGTGGGTGTAGTGGGCATGCTGGACCCTCCGCGCAAGGAGGTCACGGGCTCCATCCAGCTGT |
| 1841 | GCCGTGACGCCGGGATCCGGGTGATCATGATCACTGGGGACAACAAGGGCACAGCCATTGCCATCTGCCGGCGAATTGGC |
| 1921 | ATCTTTGGGGAGAACGAGGAGGTCGCCGATCGCGCCTACACGGGCCGAGAGTTCGACGACCTGCCCCTGGCTGAACAGCG |
| 2001 | GGAAGCCTGCCGACGTGCCTGCTGCTTCGCCCGTGTGGAGCCCTCGCACAAGTCCAAGATTGTGGAGTACCTGCAGTCCT |
| 2081 | ACGATGAGATCACAGCCATGACAGGTGATGGCGTCAATGACGCCCCTGCCCTGAAGAAGGCTGAGATTGGCATTGCCATG |
| 2161 | GGATCTGGCACTGCCGTGGCCAAGACTGCCTCTGAGATGGTGCTGGCTGACGACAACTTCTCCACCATCGTAGCTGCTGT |
| 2241 | GGAGGAGGGCCGCGCCATCTACAACAACATGAAGCAGTTCATCCGCTACCTCATTTCCTCCAACGTGGGCGAGGTGGTCT |
| 2321 | GTATCTTCCTGACCGCTGCCCTGGGGCTGCCTGAGGCCCTGATCCCGGTGCAGCTGCTATGGGTGAACTTGGTGACCGAC |
| 2401 | GGGCTCCCAGCCACAGCCCTGGGCTTCAACCCACCAGACCTGGACATCATGGACCGCCCCCCCCGGAGCCCCAAGGAGCC |
| 2481 | CCTCATCAGTGGCTGGCTCTTCTTCCGCTACATGGCAATCGGGGGCTATGTGGGTGCAGCCACCGTGGGAGCAGCTGCCT |
| 2561 | GGTGGTTCCTGTACGCTGAGGATGGGCCTCATGTCAACTACAGCCAGCTGACTCACTTCATGCAGTGCACCGAGGACAAC |
| 2641 | ACCCACTTTGAGGGCATAGACTGTGAGGTCTTCGAGGCCCCCGAGCCCATGACCATGGCCCTGTCCGTGCTGGTGACCAT |
| 2721 | CGAGATGTGCAATGCACTGAACAGCCTGTCCGAGAACCAGTCCCTGCTGCGGATGCCACCCTGGGTGAACATCTGGCTGC |
| 2801 | TGGGCTCCATCTGCCTCTCCATGTCCCTGCACTTCCTCATCCTCTATGTTGACCCCCTGCCGATGATCTTCAAGCTCCGG |
| 2881 | GCCCTGGACCTCACCCAGTGGCTCATGGTCCTCAAGATCTCACTGCCAGTCATTGGGCTCGACGAAATCCTCAAGTTCGT |
| 2961 | TGCTCGGAACTACCTAGAGGATCCAGAAGATGAAAGAAGGAAGTGAGCATCCTTTTGCTCTGTCCTCCCCACCCCGATAG |

TABLE I5
>CG57417-03-prot 1001 aa

(SEQ ID NO:587)

MEAAHAKTTEECLAYFGVSETTGLTPDQVKRNLEKYGLNELPAEEGKTLWELVIEQFEDLLVRILLLAACISFVLAWFEE
GEETITAFVEPFVILLILIANAIVGVWQERNAENAIEALKEYEPEMGKVYRADRKSVQRIKARDIVPGDIVEVAVGDKVP
ADIRILAIKSTTLRVDQSILTGESVSVIKHTEPVPDPRAVNQDKKNMLFSGTNIAAGKALGIVATTGVGTEIGKIRDQMA
ATEQDKTPLQQKLDEFGEQLSKVISLICVAVWLINIGHFNDPVHGGSWFRGAIYYFKIAVALAVAAIPEGLPAVITTCLA
LGTRRMAKKNAIVRSLPSVETLGCTSVICSDKTGTLTTNQMSVCKMFIIDKVDGDICLLNEFSITGSTYAPEGEVLKNDK
PVRPGQYDGLVELATICALCNDSSLDFNEAKGVYEKVGEATETALTTLVEKMNVFNTDVRSLSKVERANACNSVIRQLMK
KEFTLEFSRDRKSMSVYCSPAKSSRAAVGNKMFVKGAPEGVIDRCNYVRVGTTRVPLTGPVKEKIMAVIKEWGTGRDTLR
CLALATRDTPPKREEMVLDDSARFLEYETDLTFVGVVGMLDPPRKEVTGSIQLCRDAGIRVIMITGDNKGTAIAICRRIG
IFGENEEVADRAYTGREFDDLPLAEQREACRRACCFARVEPSHKSKIVEYLQSYDEITAMTGDGVNDAPALKKAEIGIAM
GSGTAVAKTASEMVLADDNFSTIVAAVEEGRAIYNNMKQFIRYLISSNVGEVVCIFLTAALGLPEALIPVQLLWVNLVTD
GLPATALGFNPPDLDIMDRPPRSPKEPLISGWLFFRYMAIGGYVGAATVGAAAWWFLYAEDGPHVNYSQLTHFMQCTEDN
THFEGIDCEVFEAPEPMTMALSVLVTIEMCNALNSLSENQSLLRMPPWVNIWLLGSICLSMSLHFLILYVDPLPMIFKLR
ALDLTQWLMVLKISLPVIGLDEILKFVARNYLEDPEDERRK

Human SERCA1 Adult Isoform: 1001 Amino Acids; 110 kd; Locus: 12q24.1; Integral Membrane Protein SR

The following is an alignment of the protein sequences of the human adult and neonatal form of SERCA1 and the rat and mouse versions of the SERCA1. For the mouse there is only a partial public sequence available.

[image]

In addition to the human version of the SERCA1 adult isoform identified as being differentially expressed in the experimental study, one variants have been identified by direct sequencing of cDNAs derived from many different human tissues and from sequences in public databases. This is the splice variant known in the public database as the neonatal isoform of SERCA1 (see above for clustalW). No splice-form variants have been identified at CuraGen whereas several amino acid-changing cSNPs were identified. These are found below. The preferred variant of all those identified, to be used for screening purposes, is CG57417-03.

TABLE I7

The variants of the human SERCA1 adult isoform
obtained from direct cloning and/or public databases

| | | | | | | |
|---|---|---|---|---|---|---|
| | DNA | | | | AA | AA |
| SNP ID | Position | E−Value | Strand | Alleles | Position | Change |

| | | | | | | |
|---|---|---|---|---|---|---|
| 13375096 | 1258 | 7.40E−06 | Plus | T:C | 420 | Cys => Arg |
| 13375098 | 1625 | 7.40E−06 | Minus | A:G | 542 | Lys => Arg |
| 13375097 | 1943 | 7.40E−06 | Plus | T:C | 648 | Val => Ala |
| 13374986 | 2199 | 7.40E−06 | Plus | G:A | 733 | Met => Ile |
| 13374987 | 2213 | 7.40E−06 | Plus | A:G | 738 | Asp => Gly |
| 13374988 | 2269 | 7.40E−06 | Plus | A:G | 757 | Met => Val |
| 13374989 | 2284 | 7.40E−06 | Plus | C:T | 762 | Arg => Cys |

Expression of gene CG57417-03 was assessed using the primer-probe set Ag3267, described in Table I8a. Results of the RTQ-PCR runs are shown in Tables I8b and I8C.

CG57417-03: SERCA1—isoform1 (neonatal), clone status=FIS; novelty=Public; ORF start=3, ORF stop=3096, frame=3; 3454 bp.

The probe and primers were designed on the neonatal isoform of the human SERCA1 gene in the non-coding region. This noncoding region is not included in CG57417-03 (see clustalW below) but is considered the same for the two alternative spliced forms of the gene. Primers both recognize the adult and neonatal SERCA1 isoforms.

[image]

[image]

[image]

[image]

Expression data was analyzed as described in Example C.

TABLE I8a
Primers and probe for Ag3267

| | | | | |
|---|---|---|---|---|
| | | | Start | SEQ ID |
| Primers | Sequences | Length | Position | NO: |

| | | | | |
|---|---|---|---|---|
| Forward | 5′-ccctctcaaccttgtaaattccc-3′ | 23 | 3313 | 595 |
| Probe | TET-5′-ttgcagggacaaggcgaccga-3′-TAMRA | 21 | 3355 | 596 |
| Reverse | 5′-aataaataagcagctcagcgca-3′ | 22 | 3377 | 597 |

TABLE I8b
General_screening_panel_v1.4

| | Rel. | Rel. |
|---|---|---|
| | Exp. (%) | Exp. (%) |
| | Ag3267, | Ag3267, |
| | Run | Run |
| Tissue Name | 208010012 | 212650192 |
| Adipose | 2.5 | 0.3 |
| Melanoma* Hs688(A).T | 0.0 | 0.1 |
| Melanoma* Hs688(B).T | 0.0 | 0.0 |
| Melanoma* M14 | 0.1 | 0.1 |
| Melanoma* LOXIMVI | 0.0 | 0.0 |
| Melanoma* SK-MEL-5 | 0.1 | 0.1 |
| Squamous cell carcinoma SCC-4 | 0.0 | 0.0 |
| Testis Pool | 0.2 | 0.1 |
| Prostate ca.* (bone met) PC-3 | 0.1 | 0.1 |
| Prostate Pool | 0.1 | 0.2 |
| Placenta | 0.1 | 0.1 |
| Uterus Pool | 0.0 | 0.0 |
| Ovarian ca. OVCAR-3 | 0.1 | 0.2 |
| Ovarian ca. SK-OV-3 | 0.2 | 0.3 |
| Ovarian ca. OVCAR-4 | 0.0 | 0.1 |
| Ovarian ca. OVCAR-5 | 0.3 | 0.3 |
| Ovarian ca. IGROV-1 | 0.0 | 0.0 |
| Ovarian ca. OVCAR-8 | 0.0 | 0.1 |
| Ovary | 0.0 | 0.1 |
| Breast ca. MCF-7 | 0.1 | 0.2 |
| Breast ca. MDA-MB-231 | 0.1 | 0.2 |
| Breast ca. BT 549 | 0.1 | 0.2 |
| Breast ca. T47D | 0.2 | 0.4 |
| Breast ca. MDA-N | 0.1 | 0.1 |
| Breast Pool | 0.1 | 0.1 |
| Trachea | 0.2 | 0.2 |
| Lung | 0.1 | 0.1 |
| Fetal Lung | 0.1 | 0.2 |
| Lung ca. NCI-N417 | 0.0 | 0.0 |
| Lung ca. LX-1 | 0.4 | 0.4 |
| Lung ca. NCI-H146 | 0.1 | 0.1 |
| Lung ca. SHP-77 | 0.2 | 0.3 |
| Lung ca. A549 | 0.1 | 0.2 |
| Lung ca. NCI-H526 | 0.0 | 0.0 |
| Lung ca. NCI-H23 | 0.2 | 0.3 |
| Lung ca. NCI-H460 | 0.1 | 0.1 |
| Lung ca. HOP-62 | 0.1 | 0.1 |
| Lung ca. NCI-H522 | 0.3 | 0.3 |
| Liver | 0.0 | 0.0 |
| Fetal Liver | 0.0 | 0.1 |
| Liver ca. HepG2 | 0.4 | 0.4 |
| Kidney Pool | 0.1 | 0.2 |
| Fetal Kidney | 0.3 | 0.2 |
| Renal ca. 786-0 | 0.1 | 0.1 |
| Renal ca. A498 | 0.1 | 0.1 |
| Renal ca. ACHN | 0.1 | 0.2 |
| Renal ca. UO-31 | 0.1 | 0.1 |
| Renal ca. TK-10 | 0.5 | 0.7 |
| Bladder | 0.3 | 0.3 |
| Gastric ca. (liver met.) NCI-N87 | 1.4 | 1.4 |
| Gastric ca. KATO III | 0.3 | 0.3 |
| Colon ca. SW-948 | 0.0 | 0.0 |
| Colon ca. SW480 | 0.1 | 0.2 |
| Colon ca.* (SW480 met) SW620 | 0.1 | 0.2 |
| Colon ca. HT29 | 0.1 | 0.1 |
| Colon ca. HCT-116 | 0.3 | 0.4 |
| Colon ca. CaCo-2 | 0.2 | 0.2 |
| Colon cancer tissue | 0.1 | 0.1 |
| Colon ca. SW1116 | 0.1 | 0.1 |
| Colon ca. Colo-205 | 0.0 | 0.1 |
| Colon ca. SW-48 | 0.0 | 0.1 |
| Colon Pool | 0.1 | 0.6 |
| Small Intestine Pool | 0.2 | 0.2 |
| Stomach Pool | 0.1 | 0.2 |
| Bone Marrow Pool | 0.0 | 0.0 |
| Fetal Heart | 0.1 | 0.1 |
| Heart Pool | 0.0 | 0.0 |
| Lymph Node Pool | 0.1 | 0.2 |
| Fetal Skeletal Muscle | 16.7 | 17.0 |
| Skeletal Muscle Pool | 100.0 | 100.0 |
| Spleen Pool | 0.2 | 0.2 |
| Thymus Pool | 0.1 | 0.2 |
| CNS cancer (glio/astro) U87-MG | 0.2 | 0.2 |
| CNS cancer (glio/astro) U-118-MG | 0.2 | 0.3 |
| CNS cancer (neuro; met) SK-N-AS | 0.2 | 0.4 |
| CNS cancer (astro) SF-539 | 0.1 | 0.1 |
| CNS cancer (astro) SNB-75 | 0.1 | 0.1 |
| CNS cancer (glio) SNB-19 | 0.0 | 0.0 |
| CNS cancer (glio) SF-295 | 0.3 | 0.6 |
| Brain (Amygdala) Pool | 0.0 | 0.0 |
| Brain (cerebellum) | 0.1 | 0.1 |
| Brain (fetal) | 0.2 | 0.2 |
| Brain (Hippocampus) Pool | 0.0 | 0.1 |
| Cerebral Cortex Pool | 0.0 | 0.1 |
| Brain (Substantia nigra) Pool | 0.0 | 0.0 |
| Brain (Thalamus) Pool | 0.1 | 0.1 |
| Brain (whole) | 0.1 | 0.1 |
| Spinal Cord Pool | 0.2 | 0.3 |
| Adrenal Gland | 0.1 | 0.1 |
| Pituitary gland Pool | 0.0 | 0.1 |
| Salivary Gland | 0.2 | 0.3 |
| Thyroid (female) | 0.0 | 0.0 |
| Pancreatic ca. CAPAN2 | 0.1 | 0.1 |
| Pancreas Pool | 0.2 | 0.1 |

TABLE I8c
Panel 5D

| | Rel. | | Rel. |
|---|---|---|---|
| | Exp. (%) | | Exp. (%) |
| | Ag3267, | | Ag3267, |
| | Run | | Run |
| Tissue Name | 166510707 | Tissue Name | 166510707 |
| 97457_Patient-02go_adipose | 0.0 | 94709_Donor 2 AM - A_adipose | 0.0 |
| 97476_Patient-07sk_skeletal | 6.1 | 94710_Donor 2 AM - B _adipose | 0.0 |
| muscle | | | |
| 97477_Patient-07ut_uterus | 0.0 | 94711_Donor 2 AM - C_adipose | 0.0 |
| 97478 _Patient-07pl_placenta | 0.0 | 94712_Donor 2 AD - A_adipose | 0.0 |
| 97481_Patient-08sk_skeletal | 5.3 | 94713_Donor 2 AD - B _adipose | 0.0 |
| muscle | | | |
| 97482_Patient-08ut_uterus | 0.0 | 94714_Donor 2 AD - C_adipose | 0.0 |
| 97483_Patient-08pl_placenta | 0.0 | 94742_Donor 3 U - A_Mesenchymal | 0.0 |
| | | Stem Cells | |
| 97486_Patient-09sk_skeletal | 7.4 | 94743_Donor 3 U - B_Mesenchymal | 0.1 |
| muscle | | Stem Cells | |
| 97487_Patient-09ut_uterus | 0.0 | 94730_Donor 3 AM - A_adipose | 0.0 |
| 97488_Patient-09pl_placenta | 0.1 | 94731_Donor 3 AM - B_adipose | 0.1 |
| 97492_Patient-10ut_uterus | 0.0 | 94732_Donor 3 AM - C_adipose | 0.1 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal | 20.6 | 94735_Donor 3 AD - C_adipose | 0.0 |
| muscle | | | |
| 97497_Patient-11ut_uterus | 0.2 | 77138_Liver_HepG2untreated | 0.2 |
| 97498_Patient-11pl_placenta | 0.0 | 73556_Heart_Cardiac stromal cells | 0.0 |
| | | (primary) | |
| 97500_Patient-12go_adipose | 0.0 | 81735_Small Intestine | 0.1 |
| 97501_Patient-12sk_skeletal | 100.0 | 72409_Kidney_Proximal Convoluted | 0.0 |
| muscle | | Tubule | |
| 97502_Patient-12ut_uterus | 0.0 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2 U - | 0.0 | 72410_Kidney_HRCE | 0.0 |
| A_Mesenchymal Stem Cells | | | |
| 94722_Donor 2 U - | 0.0 | 72411_Kidney_HRE | 0.1 |
| B_Mesenchymal Stem Cells | | | |
| 94723_Donor 2 U - | 0.0 | 73139_Uterus_Uterine smooth | 0.0 |
| C_Mesenchymal Stem Cells | | muscle cells | |

General_screening_panel_v1.4 Summary: Ag3267 Primers Specific for SERCA1

Biochemistry and Cell Line Expression

The following illustrations summarize the biochemistry surrounding the human SERCA1 adult isoform and potential assays that may be used to screen for antibody therapeutics or small molecule drugs to treat obesity and/or diabetes. Cell lines expressing the SERCA1 adult isoform can be obtained from the RTQ-PCR results shown above. These and other SERCA1 adult isoform expressing cell lines could be used for screening purposes.

The function of SERCA1 can be measured directly in a calcium flux assay using whole cells as well as subcellular fractionations as described in the Wheatly et al., Smith et al., and Thrower et al. references.

Alternatively, the ATPase activity of SERCA can be measured with measuring radioactive free phosphate.

Rationale for Use as a Diagnostic and/or Target for Small Molecule Drugs and Antibody Therapeutics.

The following is a summary of the findings from the discovery studies, supplementary investigations and assays that also incorporates knowledge in the scientific literature. Taken in total, the data indicates that an inhibitor/antagonist of the human SERCA1 adult isoform would be beneficial in the treatment of obesity and/or diabetes.

Table I11 shows that SERCA1 and the RYR1 have antagonistic functions in calcium signaling in the sarcoplasmic reticulum. SERCA1 catalyzes the hydrolysis of ATP coupled with the translocation of calcium from the cytosol to the lumen of the SR/ER.

In the muscle, the lean Cast/Ei mouse was found to have a mutation in SERCA1 which ablates its ATPase activity. The presence of a nonfunctional SERCA1 may lead to increased futile cycling of calcium, which may result in a leaner phenotype of these animals. Thus, an antagonist for SERCA1 may increase futile cycling and energy expenditure and could be beneficial in the treatment of obesity.

On the other hand, increased activity of SERCA1 will replenish the calcium pool for adequate excitation-contraction coupling, leading to a better exercise-dependent insulin sensitivity of the muscle. Therefore, an agonist of SERCA1 could be beneficial for the treatment of diabetes.

[image]

[image]

J. NOV29b—Human Autotaxin-t-Like Protein—CG93541-01

Discovery Process The following sections describe the study design(s) and the techniques used to identify the Autotaxin-t-encoded protein and any variants, thereof, as being suitable as diagnostic markers, targets for an antibody therapeutic and targets for a small molecule drugs for Obesity and Diabetes.

Studies:

A. Identification of Genes Expressed in Human Pancreas

B. Insulin Secretion From Clonal INS-1 Cell Lines (MB.11)

Study Statements:

The regulation of insulin secretion is critical to the control of serum glucose concentrations. Alterations in the secretion of insulin are central to the etiology, pathogenesis and consequences of both Type I and Type II diabetes. This study was designed to determine the role of gene expression in regulating insulin secretion from rat pancreatic beta cell lines derived from the heterogeneous rat INS-1 insulinoma. The rat insulinoma cell line INS-1 was transfected with the plasmid pCMV8/INS/IRES/Neo. The plasmid expresses the human insulin gene and the neo selectable marker under the control of the CMV promoter. Stable clones expressing these genes were isolated and described in Hohmeier, H E, Mulder, H., Chen, G., Prentki, M., Newgard, C B: Isolation of INS-1 derived cell lines with robust K ATP channel-dependent and independent glucose stimulated insulin secretion. Diabetes 49: 424-430, 2000.

TABLE J1
INS-1 Derived Cell Lines

| | | | Good |
|---|---|---|---|
| | | Poor Insulin | Insulin |
| Phenotypes Of The Cell Lines | | Secretion | Secretion |
| Glucagon Expression | Negative | 832/1 | 832/13 |
| | | 832/2 | 833/15 |
| | Positive | 834/105 | |
| | | 834/112 | |
| Species #1 | Humans | Strains | N/A |
| Species #2 | Rat | Strains INS-1 | Derived Cell Lines |
| Species #3 | N/A | Strains | N/A |

The bifunctional enzyme phosphodiesterase I (EC 3.1.4.1)/nucleotide pyrophosphatase (EC 3.6.1.9) was cloned from rat brain by Narita et al. (1994) and designated PD-I(alpha). Kawagoe et al. (1995) obtained the human cDNA which codes for a predicted 863-amino acid protein with 89% identity to the rat protein. Northern blot analysis detected a 3-kb transcript in brain, placenta, kidney and lung. See, Online Mendelian Inheritance in Man (“OMIM”), accession no. 601060.

Phosphodiesterase I (EC 3.1.4.1)/nucleotide pyrophosphatase (EC 3.6.1.9) enzymes are a family of type II transmembrane proteins that catalyze the cleavage of phosphodiester and phosphosulfate bonds of a variety of molecules, including deoxynucleotides, NAD, and nucleotide sugars. Two previously cloned genes for 2 members of this family were designated PC-1 (PDNP1; 173335) and PD-I-alpha/autotaxin (PDNP2; 601060). Jin-Hua et al. (1997) cloned the third member of this family from a human prostate cDNA library and designated it phosphodiesterase-I-beta (PD-I-beta). The gene is symbolized PDNP3. See, OMIM 602182.

An apparent splice variant lacking 52 amino acids, but otherwise identical, has been described (Murata et al., 1994). Kawagoe et al. (1995) obtained a genomic clone for the 5-prime end of the gene which contained a variety of potential DNA-binding sites as well as intron 1.

Method of Identifying the Differentially Expressed Gene and Gene Product

It was determined by a directed mining approach utilizing CuraGen proprietary (SeqCalling) and public databases of expressed sequences that the human Autotaxin-t (PDE1 isoform) is expressed in human pancreas.

Subsequently, a gene fragment of the rat PDE1 was initially found to be down-regulated by 3.2 fold in good insulin-secreting INS-1-derived cell lines compared to poor insulin-secreting INS-1-derived cell lines using CuraGen's GeneCalling™ method of differential gene expression.

The GeneCalling™ method makes a comparison between experimental samples in the amount of each cDNA fragment generated by digestion with a unique pair of restriction endonucleases, after linker-adaptor ligation, PCR amplification and electropherogramatic separation. Computer analysis is employed to assign potential identity to the gene fragment. Seven of 10 expected gene fragments from the rat PDE1 cDNA were identified as being down-regulated in the good versus poor secretors.

A differentially expressed rat gene fragment migrating, at approximately 419 nucleotides in length (FIGS. 1A and 1B—vertical line) was definitively identified as a component of the rat Autotaxin-t cDNA by competitive PCR as well as by PCR with Perfect or Mismatched 3′ Nucleotides (in the graphs, the abscissa is measured in lengths of nucleotides and the ordinate is measured as signal response). Four additional gene fragments were also identified by PCR with Perfect or Mismatched 3′ Nucleotides (See Below).

Three methods are routinely used in the identification of a gene fragment found to have altered expression in models of or patients with obesity and/or diabetes.

1) Direct Sequencing

The differentially expressed gene fragment is isolated, cloned into a plasmid and sequenced. Afterwards the sequence information is used to design an oligonucleotide corresponding to either or both termini of the gene fragment. This oligonucleotide, when used in a competitive PCR reaction, will ablate the chromatographic band from which the sequence is derived.

2) Competitive PCR

In competitive PCR, the electropherogramatic peaks corresponding to the gene fragment of the human Autotaxin-t are ablated when a gene-specific primer (designed from the sequenced band or available databases) competes with primers in the linker-adaptors during the PCR amplification. The peaks at 419 nt in length are ablated in the sample from both the gestational diabetics and normal patients.

3) PCR with Perfect or Mismatched 3′ Nucleotides (Trapping)

This method utilizes a competitive PCR approach using a degenerate set of primers that extend one or two nucleotides into the gene-specific region of the fragment beyond the flanking restriction sites. As in the competitive PCR approach, primers that lead to the ablation of the electropherogramatic band add additional sequence information. In conjunction with the size of the gene fragment and the 12 nucleotides of sequence derived from the restriction sites, this additional sequence data can uniquely define the gene after database analysis.

The direct sequence of the 419 nucleotide-long gene fragment and the gene-specific primers used for competitive PC are indicated on the complete cDNA sequence of the Autotaxin-t and shown below in bold.

Tables J2A and 2B show a differentially expressed rat PDE1 gene fragment from Discovery Study MB.11. The electropherograms represent the competitive PCR results for the Rat Autotaxin-t and provide confirmation of differential expression. The electropherogramatic peaks corresponding to the gene fragment of the Rat Autotaxin-t are ablated when a gene-specific primer (designed from the sequenced band or available databases; see below) competes with primers in the linker-adaptors during the PCR amplification. The peaks at 419 nt in length are ablated in the sample from the good (top) versus poor (bottom) secretors. In the graphs, the abscissa is measured in lengths of nucleotides and the ordinate is measured as signal response.

[image]

The sequence shown below is the Rat PDE1 cDNA. The gene fragment (band size: 418 nucleotides in length (migrating as 419 nt) identified as being differentially expressed corresponds to nucleotides 1257 to 1674 (bold) in the cDNA. The gene-specific primer used in the competitive PCR reaction is underlined.

TABLE J3
Rat Sequence # A55453

(fragment from 1257 to 1674 in bold. band size: 418)

(SEQ ID NO:598)

GGTACCCAAC AGCCTGAACT CAGAGCCCCG AGAGCAGAGC ATTCAGGGCA AGCAGAAACA CCCTGCAGAG
GCTTTCCAAG AATCCCTCGG CATGGCAAGA CAAGGCTGTC TCGGGTCATT CCAGGTAATA TCCTTGTTCA
CTTTTGCCAT CAGTGTCAAT ATCTGCTTAG GATTCACAGC AAGTCGAATT AAGAGGGCAG AATGGGATGA
AGGACCTCCC ACAGTGCTGT CTGACTCTCC ATGGACCAAC ACCTCTGGAT CCTGCAAAGG TAGATGCTTT
GAGCTTCAAG AGGTTGGCCC TCCAGACTGT CGGTGTGACA ACCTGTGTAA GAGCTACAGC AGCTGCTGCC
ACGATTTCGA TGAGCTCTGT TTGAAAACAG TCCGAGGCTG GGAGTGCACC AAAGACAGAA GTGGGGAAGT
ACGAAACGAG GAAAATGCCT GTCACTGCCC AGAAGACTGC TTGTCCAGGG GAGACTGCTG TACCAACTAC
CAAGTGGTCT GCAAAGGAGA ATCACACTGG GTAGATGATG CTGCGAGAAA TCAAAGTTCC GAATGCCTGC
AGGTTTGTCC GCCTCCGTTA ATCATCTTCT CTGTGGATGG TTTCCGTGCA TCATACATGA AGAAAGGCAG
CAAGGTTATG CCCAACATTG AGAAACTGCG GTCCTGTGGC ACCCATGTCC CCTACACGAG GCCTGTGTAC
CCCACAAAAA CCTTCCCTAA TCTATATACG CTGGCCACTG GTTTATATCC GGAATCCCAT GGAATTGTCG
GTAATTCAAT GTATGATCCT GTCTTTGATG CTTCGTTCCA TCTACGAGGG CGAGAGAAGT TTAATCATAG
GTGGTGGGGA GGCCAACCGC TATGGATTAC AGCCACCAAG CAAGGGGTGA GAGCTGGAAC ATTCTTTTGG
TCTGTGAGCA TCCCTCATGA ACGGAGGATC CTAACCATTC TTCAGTGGCT TTCTCTGCCA GACAACGAGA
GGCCTTCAGT TTATGCCTTC TACTCAGAGC AGCCTGATTT TTCTGGACAC AAGTACGGCC CTTTTGGCCC
TGAGATGACA AATCCTCTGA GGGAGATTGA CAAGACCGTG GGGCAGTTAA TGGATGGACT GAAACAACTC
AGGCTGCATC GCTGTGTGAA CGTTATCTTT GTTGGAGACC ATGGAATGGA AGATGTGACA TGTGACAGAA
CTGAGTTCTT GAGCAACTAT CTGACTAATG TGGATGACAT TACTTTAGTG CCTGGAACTC TGGGAAGAAT
TCGAGCCAAA TCTATCAATA ATTCTAAATA TGACCCTAAA ACCATTATTG CTAACCTCAC GTGCAAAAAA
CCGGATCAGC ACTTTAAGCC TTACATGAAA CAGCACCTTC CCAAACGGTT GCACTATGCC AACAACAGAA
GAATTGAAGA CATCCATTTA TTGGTCGATC GAAGATGGCA TGTTGCAAGG AAACCTTTGG ACGTTTATAA
GAAACCATCA GGAAAATGTT TTTTCCAGGG TGACCACGGC TTTGATAACA AGGTCAATAG CATGCAGACT
GTTTTCGTAG GTTATGGCCC AACTTTTAAG TACAGGACTA AAGTGCCTCC ATTTGAAAAC ATTGAACTTT
ACAATGTTAT GTGCGATCTC CTAGGCTTGA AGCCCGCTCC CAATAATGGA ACTCATGGAA GCTTGAATCA
CCTACTGCGT ACAAATACCT TTAGGCCAAC CATGCCAGAC GAAGTCAGCC GACCTAACTA CCCAGGGATT
ATGTACCTTC AGTCCGAGTT TGACCTGGGC TGCACCTGTG ACGATAAGGT AGAGCCAAAG AACAAATTGG
AAGAACTCAA TAAACGTCTT CATACCAAAG GATCAACAGA AGCTGAAACC GGGAAATTCA GAGGCAGCAA
ACATGAAAAC AAGAAAAACC TTAATGGAAG TGTTGAACCT AGAAAAGAGA GACATCTCCT GTATGGACGG
CCTGCAGTGC TCTATCGGAC TAGCTATGAT ATCTTATACC ATACGGACTT TGAAAGTGGT TATAGTGAAA
TATTCTTAAT GCCTCTCTGG ACATCGTATA CCATTTCTAA GCAGGCTGAG GTCTCCAGCA TCCCAGAACA
CCTGACCAAC TGTGTTCGTC CTGATGTCCG TGTGTCTCCA GGATTCAGTC AGAACTGTTT AGCTTATAAA
AATGATAAAC AGATGTCATA TGGATTCCTT TTTCCTCCCT ACCTGAGCTC CTCCCCAGAA GCTAAGTATG
ATGCATTCCT CGTAACCAAC ATGGTTCCAA TGTACCCCGC CTTCAAACGT GTTTGGGCTT ATTTCCAAAG
GGTTTTGGTG AAGAAATATG CTTCAGAAAG GAATGGAGTC AACGTAATAA GTGGACCGAT TTTTGACTAC
AATTACGATG GCCTACGTGA CACTGAAGAT GAAATTAAAC AGTATGTGGA AGGCAGCTCT ATACCTGTCC
CCACCCACTA CTACAGCATC ATCACCAGCT GCCTGGACTT CACTCAGCCT GCAGACAAGT GTGACGGTCC
CCTCTCTGTG TCTTCCTTCA TCCTTCCTCA CCGACCCGAC AATGATGAGA GCTGTAATAG CTCCGAGGAT
GAGTCGAAGT GGGTAGAGGA ACTCATGAAG ATGCACACAG CTCGGGTGCG GGACATTGAG CACCTCACTG
GTCTGGATTT CTACCGGAAG ACTAGCCGTA GCTATTCGGA AATTCTGACC CTCAAGACAT ACCTGCATAC
ATATGAGAGC GAGATTTAAC TTTCTGGGCC TGGGCAGTGT AGTCTTAGCA ACTGGTGTAT ATTTTTATAT
TGTGTTTGTA TTTATTAATT TGAACCAGGA CACAAACAAA CAAAGAAACA AACAAATAAA AAAAAAAACC
ACTTAGTATT TTAATCCTGT ACCAAATCTG ACATATTAAG CTGAATGACT GTGCTATTTT TTTTCCTTAA
TTCTTGATTT AGACAGAGTT GTGTTCTGAG CAGAGTTTAT AGTGAACACT GAGGCTCACA ATCCAAGTAG
AAGCTACGTG GATCTACAAG GTGCTGCAGG TTGAAAATTT GCATTGAGGA AATATTAGTT TTCCAGGGCA
CAGTCACCAC GTGTAGTTCT GTTCTGTTTT GAAAGACTGA TTTTGTAAAG GTGCATTCAT CTGCTGTTAA
CTTTGACAGA CATATTTATG CCTTATAGAC CAAGCTTAAA TATAATAAAT CACACATTCA GATTT

The following are alignments of the cDNA and protein sequences of the human, rat and mouse versions of PDE1/Autotaxin-t.

[image]

[image]

[image]

[image]

[image]

Intracellular—Variants of the Human Autotaxin-t are Obtained from Direct Cloning and/or Public Databases.

In addition to the human version of the Autotaxin-t identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG93541-01.

Analysis of CuraGen proprietary and public human sequence databases have permitted the identification of the single nucleotide polymorphisms listed in Table J6 below:

TABLE J6
Single Nucleotide Polymorphisms of Human Autotaxin-t

| DNA ID | Protein ID | ORF Start | ORF Stop | E-Value Cutoff |
|---|---|---|---|---|
| CG93541-01 | CG93541-01 | 60 | 2648 | 0.5 |
| SNP ID | DNA Position | Strand | Alleles | AA Position |
| c100.2799 | 903 | Plus | C:T | 282 |

One splice Variant, CG93541-02, protein has the first 175 amino acids and last 23 amino acids of Autotaxin-T (Q13822). The remaining 660 amino acids of the coding region is missing. Alignment of the cDNA sequences is shown below:

[image]

J8. Expression of the Human Autotaxin-t (CG93541-01)

Tissue expression for the human Autotaxin-t was assessed using the primer-probe set Ag4285, described in Table JAA. Results of the RTQ-PCR runs are shown in Tables JAB, JAC and JAD.

Table JAA. Probe Name Ag 3857

TABLE JAA

Probe Name Ag3857

| | | | | |
|---|---|---|---|---|
| | | | | SEQ ID |
| Primers | Sequenes | Length | Start Position | NO |

| | | | | |
|---|---|---|---|---|
| Forward | 5′-tgcctggaactctaggaagaat-3′ | 22 | 1216 | 607 |
| Probe | TET-5′-tcgatccaaatttagcaacaatgcta-3′-TAMRA | 26 | 1238 | 608 |
| Reverse | 5′-agattggcaataatggctttg-3′ | 21 | 1274 | 609 |

The highest level of expression in normal, adlt tissue is in stomach.

TABLE JAB
General_screening_panel_v1.4

| | | Rel. | Rel. |
|---|---|---|---|
| | Tissue Name | Exp. (%) | Exp. (%) |
| 1. | Adipose | 27.29 | 1.1% |
| 2. | Melanoma* Hs688(A).T | 25.88 | 3% |
| 3. | Melanoma* Hs688(B).T | 28.05 | 0.7% |
| 4. | Melanoma* M14 | 28.12 | 0.6% |
| 5. | Melanoma* LOXIMVI | 40 | 0% |
| 6. | Melanoma* SK-MEL-5 | 35.45 | 0% |
| 7. | Squamous cell carcinoma SCC-4 | 37.28 | 0% |
| 8. | Testis Pool | 28.23 | .6% |
| 9. | Prostate ca.* (bone met) PC-3 | 40 | 0% |
| 10. | Prostate Pool | 29.1 | .3% |
| 11. | Placenta | 28.07 | .7% |
| 12. | Uterus Pool | 27.09 | 1.3% |
| 13. | Ovarian ca. OVCAR-3 | 34.14 | 0% |
| 14. | Ovarian ca. SK-OV-3 | 29.02 | .3% |
| 15. | Ovarian ca. OVCAR-4 | 35.02 | 0% |
| 16. | Ovarian ca. OVCAR-5 | 40 | 0% |
| 17. | Ovarian ca. IGROV-1 | 36.57 | 0% |
| 18. | Ovarian ca. OVCAR-8 | 35.01 | 0% |
| 19. | Ovary | 27.64 | .9% |
| 20. | Breast ca. MCF-7 | 35.71 | 0% |
| 21. | Breast ca. MDA-MB-231 | 33.3 | 0% |
| 22. | Breast ca. BT 549 | 29.16 | .3% |
| 23. | Breast ca. T47D | 37.3 | 0% |
| 24. | Breast ca. MDA-N | 28.03 | .7% |
| 25. | Breast Pool | 26 | 2.8% |
| 26. | Trachea | 30.06 | .2% |
| 27. | Lung | 30.56 | .1% |
| 28. | Fetal Lung | 26.02 | 2.8% |
| 29. | Lung ca. NCI-N417 | 35.41 | 0% |
| 30. | Lung ca. X-1 | 32.01 | 0% |
| 31. | Lung ca. NCI-H146 | 40 | 0% |
| 32. | Lung ca. SHP-77 | 34.66 | 0% |
| 33. | Lung ca. A549 | 40 | 0% |
| 34. | Lung ca. NCI-H526 | 37.01 | 0% |
| 35. | Lung ca. NCI-H23 | 35.97 | 0% |
| 36. | Lung ca. NCI-H460 | 34.71 | 0% |
| 37. | Lung ca. HOP-62 | 31.79 | .1% |
| 38. | Lung ca. NCI-H522 | 34.7 | 0% |
| 39. | Liver | 32.1 | 0% |
| 40. | Fetal Liver | 27.36 | 1.1% |
| 41. | Liver ca. HepG2 | 33.74 | 0% |
| 42. | Kidney Pool | 26.99 | 1.4% |
| 43. | Fetal Kidney | 28.29 | .6% |
| 44. | Renal ca. 786-0 | 36.54 | 0% |
| 45. | Renal ca. A498 | 30.46 | .1% |
| 46. | Renal ca. ACHN | 33.84 | 0% |
| 47. | Renal ca. UO-31 | 40 | 0% |
| 48. | Renal ca. TK-10 | 34.28 | 0% |
| 49. | Bladder | 28.39 | .5% |
| 50. | Gastric ca. (liver met.) NCI-N87 | 33.27 | 0% |
| 51. | Gastric ca. KATO III | 37.42 | 0% |
| 52. | Colon ca. SW-948 | 40 | 0% |
| 53. | Colon ca. SW480 | 34.71 | 0% |
| 54. | Colon ca.* (SW480 met) SW620 | 37.32 | 0% |
| 55. | Colon ca. HT29 | 33.58 | 0% |
| 56. | Colon ca. HCT-116 | 40 | 0% |
| 57. | Colon ca. CaCo-2 | 33.89 | 0% |
| 58. | Colon cancer tissue | 28.28 | .6% |
| 59. | Colon ca. SW1116 | 35.69 | 0% |
| 60. | Colon ca. Colo-205 | 37.4 | 0% |
| 61. | Colon ca. SW-48 | 40 | 0% |
| 62. | Colon Pool | 26.08 | 2.6% |
| 63. | Small Intestine Pool | 26.74 | 1.7% |
| 64. | Stomach Pool | 20.84 | 100% |
| 65. | Bone Marrow Pool | 29.33 | .3% |
| 66. | Fetal Heart | 31.12 | .1% |
| 67. | Heart Pool | 28.56 | .5% |
| 68. | Lymph Node Pool | 27.4 | 1.1% |
| 69. | Fetal Skeletal Muscle | 29.04 | .3% |
| 70. | Skeletal Muscle Pool | 29.89 | .2% |
| 71. | Spleen Pool | 28.46 | .5% |
| 72. | Thymus Pool | 27.42 | 1% |
| 73. | CNS cancer (glio/astro) U87-MG | 26.1 | 2.6% |
| 74. | CNS cancer (glio/astro) U-118-MG | 27.39 | 1.1% |
| 75. | CNS cancer (neuro; met) SK-N-AS | 32.04 | 0% |
| 76. | CNS cancer (astro) SF-539 | 29.57 | .2% |
| 77. | CNS cancer (astro) SNB-75 | 25.88 | 3% |
| 78. | CNS cancer (glio) SNB-19 | 38.07 | 0% |
| 79. | CNS cancer (glio) SF-295 | 28.28 | .6% |
| 80. | Brain (Amygdala) Pool | 25.53 | 3.9% |
| 81. | Brain (cerebellum) | 27.1 | 1.3% |
| 82. | Brain (fetal) | 30.89 | .1% |
| 83. | Brain (Hippocampus) Pool | 25.45 | 4.1% |
| 84. | Cerebral Cortex Pool | 25.45 | 4.1% |
| 85. | Brain (Substantia nigra) Pool | 25.37 | 4.3% |
| 86. | Brain (Thalamus) Pool | 24.86 | 6.2% |
| 87. | Brain (whole) | 25.78 | 3.3% |
| 88. | Spinal Cord Pool | 24.55 | 7.6% |
| 89. | Adrenal Gland | 27.36 | 1.1% |
| 90. | Pituitary gland Pool | 29.15 | .3% |
| 91. | Salivary Gland | 31.41 | .1% |
| 92. | Thyroid (female) | 30.83 | .1% |
| 93. | Pancreatic ca. CAPAN2 | 40 | 0% |
| 94. | Pancreas Pool | 26.28 | 2.3% |

The highest level of expression in tissue relevant to obesity and/or diabetes is adipose. There is also significant expression in pancreatic islets (Sample 5).

TABLE JAC
Panel 5 Islet

| | | Rel. | Rel. |
|---|---|---|---|
| | Tissue Name | Exp.(%) | Exp.(%) |
| 1. | 97457_Patient-02go_adipose | 29.9 | 59.5% |
| 2. | 97476_Patient-O7sk_skeletal muscle | 31.62 | 18% |
| 3. | 97477_Patient-07ut_uterus | 31.12 | 25.5% |
| 4. | 97478_Patient-O7pl_placenta | 30.9 | 52.1% |
| 5. | 99167_Bayer Patient 1 | 30.06 | 53.2% |
| 6. | 97482_Patient-08ut_uterus | 31.37 | 21.5% |
| 7. | 97483_Patient-08pl_placenta | 30.69 | 34.4% |
| 8. | 97486_Patient-09sk_skeletal muscle | 34.85 | 1.9% |
| 9. | 97487_Patient-09ut_uterus | 31.32 | 22.2% |
| 10. | 97488_Patient-09pl_placenta | 30.99 | 27.9% |
| 11. | 97492_Patient-10ut_uterus | 30.43 | 41.2% |
| 12. | 97493_Patient-10pl_placenta | 29.55 | 75.8% |
| 13. | 97495_Patient-11go_adipose | 32.01 | 13.8% |
| 14. | 97496_Patient-11sk_skeletal muscle | 34.29 | 2.8% |
| 15. | 97497_Patient-11ut_uterus | 30.59 | 36.9% |
| 16. | 97498_Patient-11pl_placenta | 32.05 | 13.4% |
| 17. | 97500_Patient-12go_adipose | 30.4 | 42% |
| 18. | 97501_Patient-12sk_skeletal muscle | 32.66 | 8.8% |
| 19. | 97502_Patient-12ut_uterus | 30.54 | 38.2% |
| 20. | 97503_Patient-12pl_placenta | 31.29 | 22.7% |
| 21. | 94721_Donor 2 U - A_Mesenchymal Stem Cells | 31.3 | 22.5% |
| 22. | 94722_Donor 2 U - B_Mesenchymal Stem Cells | 32.1 | 12.9% |
| 23. | 94723_Donor 2 U - C_Mesenchymal Stem Cells | 31.12 | 25.5% |
| 24. | 94709_Donor 2 AM - A_adipose | 30.27 | 46% |
| 25. | 94710_Donor 2 AM - B_adipose | 31.33 | 22.1% |
| 26. | 94711_Donor 2 AM - C_adipose | 31.66 | 17.6% |
| 27. | 94712_Donor 2 AD - A_adipose | 29.5 | 78.5% |
| 28. | 94713_Donor 2 AD - B_adipose | 29.47 | 80.1% |
| 29. | 94714_Donor 2 AD - C_adipose | 29.15 | 100% |
| 30. | 94742_Donor 3 U - A_Mesenchymal Stem Cells | 32.1 | 12.9% |
| 31. | 94743_Donor 3 U - B_Mesenchymal Stem Cells | 30.88 | 30.1% |
| 32. | 94730_Donor 3 AM - A_adipose | 30.13 | 50.7% |
| 33. | 94731_Donor 3 AM - B_adipose | 31.14 | 25.2% |
| 34. | 94732_Donor 3 AM - C_adipose | 31.24 | 23.5% |
| 35. | 94733_Donor 3 AD - A_adipose | 29.29 | 90.8% |
| 36. | 94734_Donor 3 AD - B_adipose | 31.03 | 27.2% |
| 37. | 94735_Donor 3 AD - C_adipose | 29.51 | 77.9% |
| 38. | 77138_Liver_HepG2untreated | 33.84 | 3.9% |
| 39. | 73556_Heart_Cardiac stromal cells (primary) | 39.27 | .1% |
| 40. | 81735_Small Intestine | 32.28 | 11.4% |
| 41. | 72409_Kidney Proximal Convoluted Tubule | 35.36 | 1.4% |
| 42. | 82685_Small intestine_Duodenum | 33.75 | 4.1% |
| 43. | 90650_Adrenal_Adrenocortical adenoma | 33.45 | 5.1% |
| 44. | 72410_Kidney_HRCE | 35 | 1.7% |
| 45. | 72411_Kidney_HRE | 38.23 | .2% |
| 46. | 73139_Uterus_Uterine smooth muscle cells | 34.5 | 2.5% |

TABLE J9
Autotaxin_CG93541-01

View DNA Sequence Analysis of Autotaxin_CG93541-01
Translated Protein—Frame: 3—Nucleotide 60 to 2648
Printed 80 characters to a line

(SEQ ID NO:610)

```
AGTGCACTCCGTGAAGGCAAAGAGAACACGCTGCAAAAGGCTTTCCAATAATCCTCGACATGGCAAGGAGGAGCTCGTTC
                                                                                                                      M A R R S S F
CAGTCGTGTCAGATAATATCCCTGTTCACTTTTGCCGTTGGAGTCAATATCTGCTTAGGATTCACTGCACATCGAATTAA
Q S C Q I I S L F T F A V G V N I C L G F T A H R I K
GAGAGCAGAAGGATGGGAGGAAGGTCCTCCTACAGTGCTATCAGACTCCCCCTGGACCAACATCTCCGGATCTTGCAAGG
  R A E G W E E G P P T V L S D S P W T N I S G S C K G
GCAGGTGCTTTGAACTTCAAGAGGCTGGACCTCCTGATTGTCGCTGTGACAACTTGTGTAAGAGCTATACCAGTTGCTGC
    R C F E L Q E A G P P D C R C D N L C K S Y T S C C
CATGACTTTGATGAGCTGTGTTTGAAGACAGCCCGTGCGTGGGAGTGTACTAAGGACAGATGTGGGGAAGTCAGAAATGA
H D F D E L C L K T A R A W E C T K D R C G E V R N E
AGAAAATGCCTGTCACTGCTCAGAGGACTGCTTGGCCAGGGGAGACTGCTGTACCAATTACCAAGTGGTTTGCAAAGGAG
  E N A C H C S E D C L A R G D C C T N Y Q V V C K G E
AGTCGCATTGGGTTGATGATGACTGTGAGGAAATAAAGGCCGCAGAATGCCCTGCAGGGTTTGTTCGCCCTCCATTAATC
    S H W V D D D C E E I K A A E C P A G F V R P P L I
ATCTTCTCCGTGGATGGCTTCCGTGCATCATACATGAAGAAAGGCAGCAAAGTCATGCCTAATATTGAAAAACTAAGGTC
I F S V D G F R A S Y M K K G S K V M P N I E K L R S
TTGTGGCACACACTCTCCCTACATGAGGCCGGTGTACCCAACTAAAACCTTTCCTAACTTATACACTTTGGCCACTGGGC
  C G T H S P Y M R P V Y P T K T F P N L Y T L A T G L
TATATCCAGAATCACATGGAATTGTTGGCAATTCAATGTATGATCCTGTATTTGATGCCACTTTTCATCTGCGAGGGCGA
    Y P E S H G I V G N S M Y D P V F D A T F H L R G R
GAGAAATTTAATCATAGATGGTGGGGAGGTCAACCGCTATGGATTACAGCCACCAAGCAAGGGGTGAAAGCTGGAACATT
E K F N H R W W G G Q P L W I T A T K Q G V K A G T F
CTTTTGGTCTGTTGTCATCCCTCACGAGCGGAGAATATTAACCATATTGCAGTGGCTCACCCTGCCAGATCATGAGAGGC
  F W S V V I P H E R R I L T I L Q W L T L P D H E R P
CTTCGGTCTATGCCTTCTATTCTGAGCAACCTGATTTCTCTGGACACAAATATGGCCCTTTCGGCCCTGAGATGACAAAT
    S V Y A F Y S E Q P D F S G H K Y G P F G P E M T N
CCTCTGAGGGAAATCGACAAAATTGTGGGGCAATTAATGGATGGACTGAAACAACTAAAACTGCATCGGTGTGTCAACGT
P L R E I D K I V G Q L M D G L K Q L K L H R C V N V
CATCTTTGTCGGAGACCATGGAATGGAAGATGTCACATGTGATAGAACTGAGTTCTTGAGTAATTACCTAACTAATGTGG
  I F V G D H G M E D V T C D R T E F L S N Y L T N V D
ATGATATTACTTTAGTGCCTGGAACTCTAGGAAGAATTCGATCCAAATTTAGCAACAATGCTAAATATGACCCCAAAGCC
    D I T L V P G T L G R I R S K F S N N A K Y D P K A
ATTATTGCCAATCTCACGTGTAAAAAACCAGATCAGCACTTTAAGCCTTACTTGAAACAGCACCTTCCCAAACGTTTGCA
I I A N L T C K K P D Q H F K P Y L K Q H L P K R L H
CTATGCCAACAACAGAAGAATTGAGGATATCCATTTATTGGTGGAACGCAGATGGCATGTTGCAAGGAAACCTTTGGATG
  Y A N N R R I E D I H L L V E R R W H V A R K P L D V
TTTATAAGAAACCATCAGGAAAATGCTTTTTCCAGGGAGACCACGGATTTGATAACAAGGTCAACAGCATGCAGACTGTT
    Y K K P S G K C F F Q G D H G F D N K V N S M Q T V
TTTGTAGGTTATGGCCCAACATTTAAGTACAAGACTAAAGTGCCTCCATTTGAAAACATTGAACTTTACAATGTTATGTG
F V G Y G P T F K Y K T K V P P F E N I E L Y N V M C
TGATCTCCTGGGATTGAAGCCAGCTCCTAATAATGGGACCCATGGAAGTTTGAATCATCTCCTGCGCACTAATACCTTCA
  D L L G L K P A P N N G T H G S L N H L L R T N T F R
GGCCAACCATGCCAGAGGAAGTTACCAGACCCAATTATCCAGGGATTATGTACCTTCAGTCTGATTTTGACCTGGGCTGC
    P T M P E E V T R P N Y P G I M Y L Q S D F D L G C
ACTTGTGATGATAAGGTAGAGCCAAAGAACAAGTTGGATGAACTCAACAAACGGCTTCATACAAAAGGGTCTACAGAAGA
T C D D K V E P K N K L D E L N K R L H T K G S T E E
GAGACACCTCCTCTATGGGCGACCTGCAGTGCTTTATCGGACTAGATATGATATCTTATATCACACTGACTTTGAAAGTG
  R H L L Y G R P A V L Y R T R Y D I L Y H T D F E S G
GTTATAGTGAAATATTCCTAATGCCACTCTGGACATCATATACTGTTTCCAAACAGGCTGAGGTTTCCAGCGTTCCTGAC
    Y S E I F L M P L W T S Y T V S K Q A E V S S V P D
CATCTGACCAGTTGCGTCCGGCCTGATGTCCGTGTTTCTCCGAGTTTCAGTCAGAACTGTTTGGCCTACAAAAATGATAA
H L T S C V R P D V R V S P S F S Q N C L A Y K N D K
GCAGATGTCCTACGGATTCCTCTTTCCTCCTTATCTGAGCTCTTCACCAGAGGCTAAATATGATGCATTCCTTGTAACCA
  Q M S Y G F L F P P Y L S S S P E A K Y D A F L V T N
ATATGGTTCCAATGTATCCTGCTTTCAAACGGGTCTGGAATTATTTCCAAAGGGTATTGGTGAAGAAATATGCTTCGGAA
    M V P M Y P A F K R V W N Y F Q R V L V K K Y A S E
AGAAATGGAGTTAACGTGATAAGTGGACCAATCTTCGACTATGACTATGATGGCTTACATGACACAGAAGACAAAATAAA
R N G V N V I S G P I F D Y D Y D G L H D T E D K I K
ACAGTACGTGGAAGGCAGTTCCATTCCTGTTCCAACTCACTACTACAGCATCATCACCAGCTGTCTGGATTTCACTCAGC
  Q Y V E G S S I P V P T H Y Y S I I T S C L D F T Q P
CTGCCGACAAGTGTGACGGCCCTCTCTCTGTGTCCTCCTTCATCCTGCCTCACCGGCCTGACAACGAGGAGAGCTGCAAT
    A D K C D G P L S V S S F I L P H R P D N E E S C N
AGCTCAGAGGACGAATCAAAATOCGTACAAOAACTCATOAACATCCACACAOCTAOOCTCCCTOACATTOAACATCTCAC
S S E D E S K W V E E L M K M H T A R V R D I E H L T
CAGCCTGGACTTCTTCCGAAAGACCAGCCGCAGCTACCCAGAAATCCTGACACTCAAGACATACCTGCATACATATGAGA
  S L D F F R K T S R S Y P E I L T L K T Y L H T Y E S
GCGAGATTTAACTTTCTGAGCATCTGCAGTACAGTCTTATCAACTGGTTGTATATTTTTATATTGTTTTTGTATTTATTA
    E I
```

(SEQ ID NO:611)

ATTTGAAACCAGGACATTAAAAATGTTAGTATTTTAATCCTGTACCAAATCTGACATATTATGCCTGAATGACTCCACTG
TTTTTCTCTAATGCTTGATTTAGGTAGCCTTGTGTTCTGAGTAGAGCTTGTAATAAATACTGCAGCTTGAGTTTTTAGTG
GAAGCTTCTAAATGGTGCTGCAGATTTGATATTTGCATTGAGGAAATATTAATTTTCCAATGCACAGTTGCCACATTTAG
TCCTGTACTGTATGGAAACACTGATTTTGTAAAGTTGCCTTTATTTGCTGTTAACTGTTAACTATGACAGATATATTTAA
GCCTTATAAACCAATCTTAAACATAATAAATCACACATTCAGTTTTTTCTGGTAAAAAAAAAAAAAAAAA

Table J10a shows ExPASy table for phosphodiesterase 1 and Table J10b shows ExPASy table for nucleotide pyrophosphatase. Additional cell lines expressing the Autotaxin-t can be obtained from the RTQ-PCR results shown above. These and other Autotaxin-t expressing cell lines could be used for screening purposes.

[image]

Table J11 is a schematic of pathways relevant to obesity and/or diabetes, and suggests how alterations in expression of the human Autotaxin-t and associated gene products may function in the etiology and pathogenesis of obesity and/or diabetes. The scheme incorporates the unique findings of these discovery studies in conjunction with what has been reported in the literature. The outcome of inhibiting the action of the human Autotaxin-t would be a reduction of Insulin Resistance, a major problem in obesity and/or diabetes.

[image]

Rationale for Use as a Diagnostic and/or Target for Small Molecule Drugs and Antibody Therapeutics.

Table J12 is a summary of the findings from the discovery studies, supplementary investigations and assays that also incorporates knowledge in the scientific literature. Taken in total, the data indicates that an inhibitor/antagonist of the human Autotaxin-t would be beneficial in the treatment of obesity and/or diabetes.

TABLE J12
Indications for Use of Autotaxin-t Inhibitors/Antagonists
in Obesity and/or Diabetes.

Autotaxin-t is a gene expressed in human islets.
Autotaxin-t, like PC-1, was found to hydrolyze the type I
phosphodiesterase substrate p-nitrophenyl thymidine-5′-
monophosphate (J Biol Chem 1994 Dec 2; 269(48): 30479-84).
The rat orthologue (PDE1) was found to be down-regulated in good
insulin secreting cell lines.
An antagonist for Autotaxin-t should therefore improve insulin
secretion in diabetes.

K. NOV30b and NOV30I-Human Adenylate Kinase 3 Alpha-Like Protein—CG93735-01

Discovery Process: The following sections describe the study design(s) and the techniques used to identify the Adenylate Kinase 3 Alpha-encoded protein and any variants, thereof, as being suitable as diagnostic markers, targets for an antibody therapeutic and targets for a small molecule drugs for Obesity and Diabetes.

| | | |
|---|---|---|
| Studies: | MB.03 | Rat Type II Diabetes |
| | MB.11 | Insulin Secretion |

Study Statements: MB.03—The GK rat was developed from the non-diabetic Wistar rat and selected over many generations on the basis of abnormal glucose tolerance. The GK rat shows mild basal hyperglycemia, marked glucose intolerance and both hepatic and peripheral insulin resistance. GK rats also demonstrate basal hyperinsulinemia and impaired insulin response to glucose. GK rats develop many of the late-term complications associated with Type 2 diabetes, including vascular disorders, nephropathy and neuropathy. Tissues were removed from adult male rats and three control strains (Wistar, Brown Norway and Fischer 344) to identify the gene expression differences that underlie the pathologic state in the GK rat model of Type II Diabetes. These specific strains of rat were chosen for differential gene expression analysis because quantitative trait loci (QTL) for diabetic traits had been reported in published genetic studies. Tissues included whole brain, skeletal muscle, visceral adipose, and liver.

MB.11 The regulation of insulin secretion is critical to the control of serum glucose concentrations. Alterations in the secretion of insulin are central to the etiology, pathogenesis and consequences of both Type I and Type II diabetes. This study was designed to determine the role of gene expression in regulating insulin secretion from rat pancreatic beta cell lines derived from the heterogeneous rat INS-1 insulinoma. The rat insulinoma cell line INS-1 was transfected with the plasmid pCMV8/INS/IRES/Neo. The plasmid expresses the human insulin gene and the neo selectable marker under the control of the CMV promoter. Stable clones expressing these genes were isolated and described in Hohmeier, H E, Mulder, H., Chen, G., Prentki, M., Newgard, C B: Isolation of INS-1 derived cell lines with robust K ATP channel-dependent and independent glucose stimulated insulin secretion. Diabetes 49: 424-430, 2000.

TABLE K1
Insulin Expression of Stable Clone Lines

| | | Poor | Good |
|---|---|---|---|
| Phenotypes Of The Cell Lines | | Insulin Secretion | Insulin Secretion |
| Glucagon Expression | Negative | 832/1 | 832/13 |
| | | 832/2 | 833/15 |
| | Positive | 834/105 | |
| | | 834/112 | |

Species #1 Rat Strains GK, Brown Norway (BN), Wistar, Fischer 344
Species #2 Rat

Adenylate Kinase 3 Alpha: This enzyme is also known as guanosine triphosphate-adenylate kinase; nucleoside triphosphate-adenosine monophosphate transphosphorylase; GTP:AMP phosphotransferase. It catalyzes the following reaction in the mitochondrial matrix:

GTP+AMP→GDP+ADP

It was initially purified from beef heart mitochondria (Albrecht G J, Biochemistry 9 (1970) 2462-2770). Bovine AK3 was cloned and sequenced (Yamada M, Shahjahan M, Tanabe T, Kishi F, Nakazawa A.; J Biol Chem Nov. 15, 1998;264(32):19192-9) and found to complement an AK3 mutation in E. coli when expressed in these cells. The X-ray crystallographic structure of the bovine enzyme has been deduced (Diederichs K, Schulz G E.; Biochemistry Sep. 4, 1990;29(35):8138-44).

SPECIES #1 (GK vs. BN adipose): A gene fragment of the rat Adenylate Kinase 3 Alpha was initially found to be up-regulated by 23.1 fold in the adipose of GK rats (which are rat models of type II diabetes) relative to the control BN rats using CuraGen's GeneCalling® method of differential gene expression. A differentially expressed rat gene fragment migrating, at approximately 217.4 nucleotides in length (FIGS. 1A and 1B, panel 1—vertical line) was definitively identified as a component of the rat Adenylate Kinase 3 Alpha cDNA (in the graphs, the abscissa is measured in lengths of nucleotides and the ordinate is measured as signal response). The method of direct sequencing followed by competitive PCR to the sequence obtained was used for confirmation of gene and the trace in FIGS. 1C and 1D, panel 2, represents the ablated peak. In addition, a differentially expressed fragment migrating at approximately 217.9 nucleotides in length was found to be up-regulated 2.2 fold in the adipose of GK rats relative to the control Wistar rats, as seen in FIGS. 1E and 1F, panel 3.

The direct sequence of the 217.4 nucleotide-long gene fragment and the gene-specific primers used for competitive PCR are indicated on the cDNA sequence of the rat adenylate kinase 3 and shown below in bold.

TABLE K2
Gene Sequence

(fragment from 1 to 218 in bold. band size: 218)

(SEQ ID NO:612)

TCATGACTCGGCTGGCCCTCCATGAGCTGAAAAACCTTACCCAGTGTAGC
TGGCTGTTGGACGGATTTCCAAGGACACTTCCACAGGCAGAAGCCCTGGA
TAGAGTTTATCAGATAGACACAGTGATAAATCTCAACGTGCCCTTTGAGG
TCATTAAACAACGCCTTACTGCTCGCTGGATTCATCCTGCCAGTGGCCGA
GTTTACAACATTGAATTC

SPECIES #2 (Glucagon negative good responders vs. glucagons negative poor responders): A gene fragment of the rat Adenylate Kinase 3 Alpha was also found to be up-regulated by 2.1 fold in the glucagon negative good insulin-secreting cells relative to the poor insulin secretors using CuraGen's GeneCalling® method of differential gene expression. A differentially expressed rat gene fragment migrating, at approximately 386.4 nucleotides in length (FIGS. 1A and 1B—vertical line) was definitively identified as a component of the rat Adenylate Kinase 3 Alpha cDNA (in the graphs, the abscissa is measured in lengths of nucleotides and the ordinate is measured as signal response). The method of competitive PCR was used to ablate the peak and to confirm gene assessment which revealed that this fragment belonged to the rat Adenylate Kinase 3 Alpha gene.

The gene-specific primers used for competitive PCR are indicated on the cDNA sequence of the rat adenylate kinase 3 and shown below in bold.

TABLE K3
Gene Sequence

(fragment from 336 to 721 in bold. band size: 386)

(SEQ ID NO:613)

GCGGGAGAGCCGGGCGCCCTGGCCACCGCCCGCTTGCAGTTGCCAGCGGGCCAGGGCCTCAGAGCCTTTGAGCGCCCAGG
CCAGGCCGCAGTTCAGCGTCTGCGCAGCTTCGGCCACCGTTGCCACCATGGGGGCATCGGGGCGGCTGCTGCGCGCCGTG
ATCATGGGGGCCCCGGGCTCCGGTAAGGGCACCGTGTCGTCACGCATCACCAAACACTTCGAGCTGAAGCACCTCTCCAG
CGGGGACCTGCTCCGCCAGAACATGCTGCAGGGCACAGAAATCGGTGTGTTGGCCAAGACTTTCATTGACCAAGGAAAGC
TCATCCCGGATGATGTCATGACTCGGCTGGCCCTCCATGAGCTGAAAAACCTTACCCAGTGTAGCTGGCTGTTGGACGGA
TTTCCAAGGACACTTCCACAGGCAGAAGCCCTGGATAGAGTTTATCAGATAGACACAGTGATAAATCTCAACGTGCCCTT
TGAGGTCATTAAACAACGCCTTACTGCTCGCTGGATTCATCCTGCCAGTGGCCGAGTTTACAACATTGAATTCAACCCTC
CCAAGACTGTGGGCATCGATGACCTAACGGGAGAACCTCTGATTCAGCGTGAGGACGACAAACCAGAGACGGTGATCAAG
AGATTGAAGGCGTATGAAGCCCAGACAGAGCCGGTCCTGCAGTATTACCAGAAAAAAGGGGTGTTGGAAACATTCTCCGG
AACAGAAACCAACAAGATCTGGCCCCACGTATACTCCTTCCTGCAAATGAAAGTTCCAGAAACCATCCAAAAAGCCTCTG
TTACTCCCTGAGGAAGGCACTTGGCGGGATGAAGCAGGGCCTCCTCCACTCCTCCCCTCGCCTCTGTATTTCGAAGCTCT
TTTCCTAAGACTTCTCTGAAAATTATGATTTAGTCCTAATGGCTCTGCCTAATGAGTCAGAAACTAAGGCTGACCATGTG
TTTATCTAGTTGTCTTCCATGGATGTGCAATTCAAAACGTCAGACATGTTGAAACAAACAAACTCAGAGCACAATTAAGA
GAGCAACTGGTGGGGTTGGGGATTTAGCTCAGTGGTAGAGCGCTTGCCTAGGAAGTACAAGGCCCTGGGTTCGGTCCCCA
GCTCCGAAAAAACAAGAAAAAACAAAACAAAACAAAACAAAACAAAAAAACACATTAGGGAGAATCCTTTACTAAAGCAG
C
(gene length is 1315, only region from 1 to 1201 shown)

TABLE K 4
Human Adenylate Kinase 3 Alpha Gene Sequence

>CG93735-01 1021 nt

(SEQ ID NO:614)

ACTTCCGGGAACGCCGGGGAACCGCAGTAGCCGCCTGCTAGTGGCGCTGCTAGCCGGCCGGCGCAGGCTGCCGAGCGGGT
GAGCGCGCAGGCCAGGCCAAAGCCCTGGTACCCGCGCGGTGCGGGCCTCAGTCTGCGGCCATGGGGGCGTCGGGGCGGCT
GCTGCGAGCGGTGATCATGGGGGCCCCGGGCTCGGGCAAGGGCACCGTGTCGTCCCGCATCACTACACACTTCGAGCTGA
AGCACCTCTCCCGCGGGGACCTGCTCCGGGACAACATGCTGCGGGGCACAGAAATTGGCGTGTTAGCCCAGGCTTTCATT
GACCAAGGGAAACTCATCCCAGATTATGTCACGACTCGGCTGGCCCTTCATGAGCTGAAAAACCTCACCCAGTATAGCTG
GCTGTTGGATGGTTTTCCAAGGACACTTCCACAGGCAGAAGCCCTAGATAGAGCTTATCAGATCGACACAGTGATTAACC
TGAATGTGCCCTTTGAGGTCATTAAACAACGCCTTACTGCTCGCTGGATTCATCCCGCCAGTGGCCGAGTCTATAACATT
GAATTCAACCCTCCCAAAACTGTGGGCATTGATGACCTGACTGGGGAGCCTCTCATTCAGCGTGAGGATGATAAACCAGA
GACGGTTATCAAGAGACTAAAGGCTTATGAAGACCAAACAAAGCCAGTCCTGGAATATTACCAGAAAAAAGGGGTGTTGG
AAACATTCTCCGGAACAGAAACCAACAAGATTTGGCCCTATGTATATGCTTTCCTACAAACTAAAGTTCCACAAAGAAGC
CAGAAAGCTTCAGTTACTCCATGAGGAGAAATGTGTGTAACTATTAATAGTAAGATGGGCAAACCTCCTAGTCCTTGCAT
TTAGAAGCTGCTTTTCCTAAGACTTCTAGTATGTATGAATTCTTTGAAAATTATATTACTTTTATTTCTACTGATTTTAT
TTTGGATACTAAGGATGTGCCAAATGATTCGGATACTAAGATGCATCGTTTGAAATCATCT

TABLE K 5
Human Adenylate Kinase 3 Alpha Protein Sequence:

ORF Start: 141 ORF Stop: 822 Frame: 3
>CG93735-01-prot 227 aa

(SEQ ID NO:615)

MGASGRLLRAVIMGAPGSGKGTVSSRITTHFELKHLSRGDLLRDNMLRGT
EIGVLAQAFIDQGKLIPDYVTTRLALHELKNLTQYSWLLDGFPRTLPQAE
ALDRAYQIDTVINLNVPFEVIKQRLTARWIHPASGRVYNIEFNPPKTVGI
DDLTGEPLIQREDDKPETVIKRLKAYEDQTKPVLEYYQKKGVLETFSGTE
TNKIWPYVYAFLQTKVPQRSQKASVTP

The following is an alignment of the protein sequences of the human (CG93735-01; SEQ ID NO:616), mouse (AK3_MOUSE; SEQ ID NO:617) and rat (AK3_RAT; SEQ ID NO:618) versions of the Adenylate Kinase 3 Alpha protein. Also included are a protein annotated as similar to the human adenylate kinase 3 (Q9NPB4; SEQ ID NO:619) and a novel human protein with significant homology to adenylate kinase 3 alpha (CG56785-01; SEQ ID NO:620).

[image]

[image]

In addition to the human version of the Adenylate Kinase 3 Alpha identified as being differentially expressed in the experimental study, two other variants have been identified by direct sequencing of cDNAs derived from many different human tissues and from sequences in public databases. No splice-form variants have been identified at CuraGen. One SNP was identified and is presented in the table below. The preferred variant of all those identified, to be used for screening purposes, is CG93735-02 (SEQ ID NO:621).

[image]

TABLE K8

SNP of CG93735-01

| SNP ID | DNA Position | E−Value | Strand | Alleles | AA Position | AA Change |
|---|---|---|---|---|---|---|
| 13374782 | 625 | 1.70E−06 | Plus | A:G | 162 | Glu=> Gly |

CG93735-01: human adenylate kinase 3 alpha—isoform1; clone status = FIS; novelty = Public; ORF start = 141, ORF stop = 822, frame = 3; 1021 bp.

Human RTQ-PCR results were obtained as described in Example C. Expression of gene CG93735-01 was assessed using the primer-probe set Ag3926, described in Table KAA. Results of the RTQ-PCR runs are shown in Tables KAB and KAC.

TABLE KAA

Probe Name Ag3926

| | | | | |
|---|---|---|---|---|
| | | | Start | SEQ ID |
| Primers | Sequences | Length | Position | NO |

| | | | | |
|---|---|---|---|---|
| Forward | 5′-gtatagctggctgttggatg-3′ | 20 | 392 | 624 |
| | (SEQ ID NO:XX) | | | |
| Probe | TET-5′-ttttccaaggacacttccacaggcagaa-3′-TAMRA | 29 | 0 | 625 |
| | (SEQ ID NO:XX) | | | |
| Reverse | 5′-cgatctgataagctctatctag-3′ | 22 | 444 | 626 |
| | (SEQ Id NO:XX) | | | |

TABLE KAB
General_screening_panel_v1.4

| | Rel. Exp.(%) |
|---|---|
| | Ag3926, Run |
| Tissue Name | 214146654 |
| Adipose | 4.6 |
| Melanoma* Hs688(A).T | 6.8 |
| Melanoma* Hs688(B).T | 5.2 |
| Melanoma* M14 | 6.1 |
| Melanoma* LOXIMVI | 4.2 |
| Melanoma* SK-MEL-5 | 4.9 |
| Squamous cell carcinoma SCC-4 | 1.7 |
| Testis Pool | 1.2 |
| Prostate ca.* (bone met) PC-3 | 5.5 |
| Prostate Pool | 3.0 |
| Placenta | 1.9 |
| Uterus Pool | 1.7 |
| Ovarian ca. OVCAR-3 | 25.5 |
| Ovarian ca. SK-OV-3 | 2.9 |
| Ovarian ca. OVCAR-4 | 2.4 |
| Ovarian ca. OVCAR-5 | 11.0 |
| Ovarian ca. IGROV-1 | 4.0 |
| Ovarian ca. OVCAR-8 | 3.0 |
| Ovary | 3.0 |
| Breast ca. MCF-7 | 4.8 |
| Breast ca. MDA-MB-231 | 3.0 |
| Breast ca. BT 549 | 13.6 |
| Breast ca. T47D | 17.1 |
| Breast ca. MDA-N | 2.3 |
| Breast Pool | 4.0 |
| Trachea | 3.0 |
| Lung | 1.4 |
| Fetal Lung | 3.5 |
| Lung ca. NCI-N417 | 1.3 |
| Lung ca. LX-1 | 5.2 |
| Lung ca. NCI-H146 | 1.8 |
| Lung ca. SHP-77 | 1.4 |
| Lung ca. A549 | 7.2 |
| Lung ca. NCI-H526 | 2.8 |
| Lung ca. NCI-H23 | 2.7 |
| Lung ca. NCI-H460 | 2.8 |
| Lung ca. HOP-62 | 6.5 |
| Lung ca. HCI-H522 | 7.0 |
| Liver | 1.4 |
| Fetal Liver | 4.0 |
| Liver ca. HepG2 | 10.0 |
| Kidney Pool | 100.0 |
| Fetal Kidney | 3.2 |
| Renal ca. 786-0 | 4.3 |
| Renal ca. A498 | 2.5 |
| Renal ca. ACHN | 3.6 |
| Renal ca. UO-31 | 5.6 |
| Renal ca. TK-10 | 6.2 |
| Bladder | 5.0 |
| Gastric ca. (liver met.) NCI-N87 | 12.0 |
| Gastric ca. KATO III | 10.7 |
| Colon ca. SW-948 | 1.3 |
| Colon ca. SW480 | 24.5 |
| Colon ca.* (SW480 met) SW620 | 6.7 |
| Colon ca. HT29 | 4.9 |
| Colon ca. HCT-116 | 6.1 |
| Colon ca. CaCo-2 | 8.7 |
| Colon cancer tissue | 3.5 |
| Colon ca. SW1116 | 1.2 |
| Colon ca. Colo-205 | 1.4 |
| Colon ca. SW-48 | 2.4 |
| Colon Pool | 100.0 |
| Small Intestine Pool | 4.9 |
| Stomach Pool | 2.7 |
| Bone Marrow Pool | 2.8 |
| Fetal Heart | 1.6 |
| Heart Pool | 63.3 |
| Lymph Node Pool | 5.9 |
| Fetal Skeletal Muscle | 2.4 |
| Skeletal Muscle Pool | 12.2 |
| Spleen Pool | 2.3 |
| Thymus Pool | 3.5 |
| CNS cancer (glio/astro) U87-MG | 5.7 |
| CNS cancer (glio/astro) U-118-MG | 5.6 |
| CNS cancer (neuro;met) SK-N-AS | 3.0 |
| CNS cancer (astro) SF-539 | 2.4 |
| CNS cancer (astro) SNB-75 | 6.7 |
| CNS cancer (glio) SNB-19 | 3.4 |
| CNS cancer (glio) SF-295 | 17.3 |
| Brain (Amygdala) Pool | 2.0 |
| Brain (cerebellum) | 2.3 |
| Brain (fetal) | 4.1 |
| Brain (Hippocampus) Pool | 3.0 |
| Cerebral Cortex Pool | 2.5 |
| Brain (Substantia nigra) Pool | 2.3 |
| Brain (Thalamus) Pool | 3.6 |
| Brain (whole) | 1.7 |
| Spinal Cord Pool | 3.0 |
| Adrenal Gland | 5.9 |
| Pituitary gland Pool | 0.7 |
| Salivary Gland | 1.4 |
| Thyroid (female) | 1.4 |
| Pancreatic ca. CAPAN2 | 4.5 |
| Pancreas Pool | 4.7 |

TABLE KAC
Panel 5 Islet

| | Rel. Exp.(%) |
|---|---|
| | Ag3926, Run |
| Tissue Name | 227742519 |
| 97457_Patient-02go_adipose | 29.7 |
| 97476_Patient-07sk_skeletal muscle | 22.1 |
| 97477_Patient-07ut_uterus | 16.3 |
| 97478_Patient-07pl_placenta | 29.3 |
| 99167_Bayer Patient 1 | 18.4 |
| 97482_Patient-08ut_uterus | 12.5 |
| 97483_Patient-08pl_placenta | 38.2 |
| 97486_Patient-09sk_skeletal muscle | 16.5 |
| 97487_Patient-09ut_uterus | 36.1 |
| 97488_Patient-09pl_placenta | 13.0 |
| 97492_Patient-10ut_uterus | 25.0 |
| 97493_Patient-10pl_placenta | 39.5 |
| 97495_Patient-11go_adipose | 28.5 |
| 97496_Patient-11sk_skeletal muscle | 49.3 |
| 97497_Patient-11ut_uterus | 31.4 |
| 97498_Patient-11pl_placenta | 17.7 |
| 97500_Patient-12go_adipose | 24.1 |
| 97501_Patient-12sk_skeletal muscle | 78.5 |
| 97502_Patient-12ut_uterus | 26.8 |
| 97503_Patient-12pl_placenta | 18.7 |
| 94721_Donor 2 U - | 17.6 |
| A_Mesenchymal Stem Cells | |
| 94722_Donor 2 U - | 12.1 |
| B_Mesenchymal Stem Cells | |
| 94723_Donor 2 U - | 18.6 |
| C_Mesenchymal Stem Cells | |
| 94709_Donor 2 AM - A_adipose | 26.4 |
| 94710_Donor 2 AM - B_adipose | 17.4 |
| 94711_Donor 2 AM - C_adipose | 14.4 |
| 94712_Donor 2 AD - A_adipose | 40.1 |
| 94713_Donor 2 AD - B_adipose | 42.3 |
| 94714_Donor 2 AD - C_adipose | 36.3 |
| 94742_Donor 3 U - A_Mesenchymal | 4.7 |
| Stem Cells | |
| 94743_Donor 3 U - B_Mesenchymal | 12.0 |
| Stem Cells | |
| 94730_Donor 3 AM - A_adipose | 24.7 |
| 94731_Donor 3 AM - B_adipose | 10.8 |
| 94732_Donor 3 AM - C_adipose | 14.2 |
| 94733_Donor 3 AD - A_adipose | 36.1 |
| 94734_Donor 3 AD - B_adipose | 13.0 |
| 94735_Donor 3 AD - C_adipose | 33.2 |
| 77138_Liver_HepG2untreated | 100.0 |
| 73556_Heart_Cardiac stromal cells (primary) | 15.7 |
| 81735_Small Intestine | 41.5 |
| 72409_Kidney_Proximal Convoluted Tubule | 21.3 |
| 82685_Small intestine_Duodenum | 29.1 |
| 90650_Adrenal_Adrenocortical adenoma | 15.3 |
| 72410_Kidney_HRCE | 40.6 |
| 72411_Kidney_HRE | 39.8 |
| 73139_Uterus_Uterine smooth muscle cells | 19.3 |

General_screening_panel_v1.4 Summary: The expression of the adenylate kinase 3 alpha gene is ubiquitous, showing high levels of expression in both normal and disease tissues. However, it is especially high in the kidney and colon pools, with lesser amounts in the heart and skeletal muscle. Among cancer cell lines, highest expression is seen in the ovarian cancer cell line OVCAR-3, with lower levels in colon cancer, breast cancer and glioblastoma cell lines.

Panel 5 Islet Summary: Expression of the adenylate kinase 3 alpha gene is ubiquitous in panel 5i, consistent with expression in panel 1.4. Highest expression is seen in the HepG2 cell line. Among human tissue samples, the highest expression is seen in skeletal muscle from patient 12.

Biochemistry and Cell Line Expression. The reaction that Adenylate Kinase 3 Alpha catalyzes is: GTP+AMP→GDP+ADP

The enzyme can be overexpressed using tagged expression constructs in E. coli, mammalian or baculovirus systems and be purified using affinity chromatography to the tag. Alternatively, conventional chromatographic techniques can be used. Successful expression in E. coli has been previously demonstrated (Yamada et al.) A well-defined AMP-binding site has been defined in X-ray crystallographic studies (Diederichs et al.) and should be clearly amenable to high-throughput screening assays. The assays can be coupled to detection systems monitoring ADP production, for example, by utilizing loss of NADH coupled through pyruvate kinase and lactate dehydrogenase.

Rationale for Use as a Diagnostic and/or Target for Small Molecule Drugs and Antibody Therapeutics.

The following is a summary of the findings from the discovery studies, supplementary investigations and assays that also incorporates knowledge in the scientific literature. Taken in total, the data indicates that an inhibitor/antagonist of the human Adenylate Kinase 3 Alpha would be beneficial in the treatment of obesity and/or diabetes.

The overexpression of the phosphotransferase AK3 in the adipocytes of the diabetic GK rat suggests a shift in mitochondrial energy production. In parallel, levels of AK3 are increased in the subset of INS-1 cells that are good secretors. Inhibition of AK3 can cause an increase in AMP levels, which could result in activation of AMP kinase, one of the key intracellular mediators of insulin signaling. Inhibition of this enzyme in pancreatic islets may, therefore, result in altered insulin secretion and may be an effective therapeutic for diabetes. AMP kinase can also phosphorylate and inactivate acetyl-CoA carboxylase (ACC), which results in a decrease in malonyl-CoA production and, as a consequence, causes an increase in fatty acid oxidation in adipose tissue. Knock-outs of ACC2, for example, have decreased body weight even though they have increased food intake (Abu-Elheiga et al). Therefore, inhibitors of AK3 may be effective therapeutics against obesity.

Methods of Use for the Compositions of the Invention

The protein similarity information, expression pattern, cellular localization, and map location for the protein and nucleic acid disclosed herein suggest that this protein may have important structural and/or physiological functions characteristic of the Adenylate Kinase 3 Alpha family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed.

The nucleic acids and proteins of the invention have applications in the diagnosis and/or treatment of various diseases and disorders. For example, the compositions of the present invention will have efficacy for the treatment of patients suffering from: obesity and/or diabetes.

These materials are further useful in the generation of antibodies that bind immunospecifically to the substances of the invention for use in diagnostic and/or therapeutic methods.

Table K10A, 10B, 10C, 10D, 10E and 10F show results disclosing differentially expressed rat adenylate kinase 3 alpha gene fragment from discovery study MB.03, species #1.

[image]

[image]

[image]

Physical cDNA Clone Available for Expression and Screening Purposes

Materials and Methods were performed as describe in Example B with exon linking and in-frame cloning.

In Frame Cloning: In frame cloning is a process designed to insert DNA sequences into expression vectors such that the encoded proteins can be produced. The expressed proteins were either full length or corresponding to specific domains of interest. The PCR template was based on a previously identified plasmid (the PCR product derived by exon linking, covering the entire open reading frame) when available, or on human cDNA(s). The human cDNA pool was composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus. For downstream cloning purposes, the forward and reverse primers included in-frame BamHI and NotI restriction sites. The amplified product was detected by agarose gel electrophoresis. The fragment was gel-purified and ligated into the pGEX-6P-1, pFastBac1, pcDNA3.1+ and pET-28a (+) (Invitrogen, Carlsbad, Calif.) following the manufacturer's recommendation. Twenty four clones per transformation were picked and a quality control step was performed to verify that these clones contain an insert of the anticipated size. Subsequently, eight of these clones were sequenced, and assembled in a fashion similar to the SeqCalling process. In addition to analysis of the entire sequence assembly, sequence traces were evaluated manually.

Results and Discussion: Table K12 depicts the preferred cDNA(s) that encompass the coding portion of the human AK3 alpha for expression of recombinant protein from any number of plasmid, phage or phagemid vectors in a variety of cellular systems for screening purposes. The corresponding amino acid sequence(s) are also listed. Although the sequences below are the preferred isoforms, any of the other isoforms may be used for similar purposes. Furthermore, under varying assay conditions, conditions may dictate that another isoform may supplant the listed isoforms. As seen in Table K13 the open reading frame of the working representatives of CG93735-01 differs with a few amino acids from CG93735-01. The CG93735-03 and CG93735-04 working representatives have N-terminal and C-terminal Histidine tags used for protein purification.

TABLE K12
Physical cDNA Clone Available for Expression & Screening Purposes

>CG93735-02 688

(SEQ ID NO:627)

ntCACCATGGGGGCGTCGGGGCGGCTGCTGCGAGCGGTGATCATGGGGGCCCCGGGCTCGGGCAAGGGCACCGTGTCGTC
GCGCATCACTACACACTTCGAGCTGAAGCACCTCTCCAGCGGGGACCTGCTCCGGGACAACATGCTGCGGGGCACAGAAA
TTGGCGTGTTAGCCAAGGCTTTCATTGACCAAGGGAAACTCATCCCAGATGATGTCATGACTCGGCTGGCCCTTCATGAG
CTGAAAAATCTCACCCAGTATAGCTGGCTGTTGGATGGTTTTCCAAGGACACTTCCACAGGCAGAAGCCCTAGATAGAGC
TTATCAGATCGACACAGTGATTAACCTGAATGTGCCCTTTGAGGTCATTAAACAACGCCTTACTGCTCGCTGGATTCATC
CCGCCAGTGGCCGAGTCTATAACATTGAATTCAACCCTCCCAAAACTGTGGGCATTGATGACCTGACTGGGGAGCCTCTC
ATTCAGCGTGAGGATGATAAACCAGAGACGGTTATCAAGAGACTAAAGGCTTATGAAGACCAAACAAAGCCAGTCCTGGA
ATATTACCAGAAAAAAGGGGTGCTGGAAACATTCTCCGGAACAGAAACCAACAAGATTTGGCCCTATGTATATGCTTTCC
TACAAACTAAAGTTCCACAAAGAAGCCAGAAAGCTTCAGTTACTCCATGA
>CG93735-02-prot 227

(SEQ ID NO:628)

aaMGASGRLLRAVIMGAPGSGKGTVSSRITTHFELKHLSSGDLLRDNMLRGTEIGVLAKAFIDQGKLIPDDVMTRLALHE
LKNLTQYSWLLDGFPRTLPQAEALDRAYQIDTVINLNVPFEVIKQRLTARWIHPASGRVYNIEFNPPKTVGIDDLTGEPL
IQREDDKPETVIKRLKAYEDQTKPVLEYYQKKGVLETFSGTETNKIWPYVYAFLQTKVPQRSQKASVTP
>CG93735-03, 709

(SEQ ID NO:629)

ntCCACCATGGGCCACCATCACCACCATCACGGGGCGTCGGGGCGGCTGCTGCGAGCGGTGATCATGGGGGCCCCGGGCT
CGGGCAAGGGCACCGTGTCGTCGCGCATCACTACACACTTCGAGCTGAAGCACCTCTCCAGCGGGGACCTGCTCCGGGAC
AACATGCTGCGGGGCACAGAAATTGGCGTGTTAGCCAAGGCTTTCATTGACCAAGGGAAACTCATCCCAGATGATGTCAT
GACTCGGCTGGCCCTTCATGAGCTGAAAAATCTCACCCAGTATAGCTGGCTGTTGGATGGTTTTCCAAGGACACTTCCAC
AGGCAGAAGCCCTAGATAGAGCTTATCAGATCGACACAGTGATTAACCTGAATGTGCCCTTTGAGGTCATTAAACAACGC
CTTACTGCTCGCTGGATTCATCCCGCCAGTGGCCGAGTCTATAACATTGAATTCAACCCTCCCAAAACTGTGGGCATTGA
TGACCTGACTGGGGAGCCTCTCATTCAGCGTGAGGATGATAAACCAGAGACGGTTATCAAGAGACTAAAGGCTTATGAAG
ACCAAACAAAGCCAGTCCTGGAATATTACCAGAAAAAAGGGGTGCTGGAAACATTCTCCGGAACAGAAACCAACAAGATT
TGGCCCTATGTATATGCTTTCCTACAAACTAAAGTTCCACAAAGAAGCCAGAAAGCTTCAGTTACTCCATA
>CG93735-03-prot 235

(SEQ ID NO:630)

aaTMGHHHHHHGASGRLLRAVIMGAPGSGKGTVSSRITTHFELKHLSSGDLLRDNMLRGTEIGVLAKAFIDQGKLIPDDV
MTRLALHELKNLTQYSWLLDGFPRTLPQAEALDRAYQIDTVINLNVPFEVIKQRLTARWIHPASGRVYNIEFNPPKTVGI
DDLTGEPLIQREDDKPETVIKRLKAYEDQTKPVLEYYQKKGVLETFSGTETNKIWPYVYAFLQTKVPQRSQKASVTP
>CG93735-04, 707

(SEQ ID NO:631)

ntTCAGTGATGGTGGTGATGGTGTGGAGTAACTGAAGCTTTCTGGCTTCTTTGTGGAACTTTAGTTTGTAGGAAAGCATA
TACATAGGGCCAAATCTTGTTGGTTTCTGTTCCGGAGAATGTTTCCAGCACCCCTTTTTTCTGGTAATATTCCAGGACTG
GCTTTGTTTGGTCTTCATAAGCCTTTAGTCTCTTGATAACCGTCTCTGGTTTATCATCCTCACGCTGAATGAGAGGCTCC
CCAGTCAGGTCATCAATGCCCACAGTTTTGGGAGGGTTGAATTCAATGTTATAGACTCGGCCACTGGCGGGATGAATCCA
GCGAGCAGTAAGGCGTTGTTTAATGACCTCAAAGGGCACATTCAGGTTAATCACTGTGTCGATCTGATAAGCTCTATCTA
GGGCTTCTGCCTGTGGAAGTGTCCTTGGAAAACCATCCAACAGCCAGCTATACTGGGTGAGATTTTTCAGCTCATGAAGG
GCCAGCCGAGTCATGACATCATCTGGGATGAGTTTCCCTTGGTCAATGAAAGCCTTGGCTAACACGCCAATTTCTGTGCC
CCGCAGCATGTTGTCCCGGAGCAGGTCCCCGCTGGAGAGGTGCTTCAGCTCGAAGTGTGTAGTGATGCGCGACGACACGG
TGCCCTTGCCCGAGCCCGGGGCCCCCATGATCACCGCTCGCAGCAGCCGCCCCGACGCCCCCATGGTGG
>CG93735-04-prot 234

(SEQ ID NO:632)

aaTMGASGRLLRAVIMGAPGSGKGTVSSRITTHFELKHLSSGDLLRDNMLRGTEIGVLAKAFIDQGKLIPDDVMTRLALH
ELKNLTQYSWLLDGFPRTLPQAEALDRAYQIDTVINLNVPFEVIKQRLTARWIHPASGRVYNIEFNPPKTVGIDDLTGEP
LIQREDDKPETVIKRLKAYEDQTKPVLEYYQKKGVLETFSGTETNKIWPYVYAFLQTKVPQRSQKASVTPHHHHHH

[image]

L. NOV31a—GPCR Olfactory Receptor-Like Protein AdEn-GPCR1-isoform 1—CG93817-01:

CG93817-01 was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof.

Methods of Use for the Compositions of the Invention

The protein similarity information, expression pattern, cellular localization, and map location for the protein and nucleic acid disclosed herein suggest that this protein may have important structural and/or physiological functions characteristic of the Human Neutral Amino Acid Transporter B family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. The nucleic acids and proteins of the invention have applications in the diagnosis and/or treatment of various diseases and disorders. For example, the compositions of the present invention will have efficacy for the treatment of patients suffering from: obesity and/or diabetes.

TABLE L1

CG93817-01 DNA Sequence
Full Length Clone Acc. No.: GMAC072059_G; also known as AdEn-GPCR1-isoform1.

>CG93817-01 999 nt

(SEQ ID NO:637)

AGGTGAACATAACATAAAAAAATGTTCCCGGCAAATTGGACATCTGTAAAAGTATTTTTCTTCCTGGGATTTTTTCACTA
CCCCAAAGTTCAGGTCATCATATTTGCGGTGTGCTTGCTGATGTACCTGATCACCTTGCTGGGCAACATTTTTCTGATCT
CCATCACCATTCTAGATTCCCACCTGCACACCCCTATGTACCTCTTCCTCAGCAATCTCTCCTTTCTGGACATCTGGTAC
TCCTCTTCTGCCCTCTCTCCAATGCTGGCAAACTTTGTTTCAGGGAGAAACACTATTTCATTCTCAGGGTGCGCCACTCA
GATGTACCTCTCCCTTGCCATGGGCTCCACTGAGTGTGTGCTCCTGCCCATGATGGCATATGACCGGTATGTGGCCATCT
GCAACCCCCTGAGATACCCTGTCATCATGAATAGGAGAACCTGTGTGCAGATTGCAGCTGGCTCCTGGATGACAGGCTGT
CTCACTGCCATGGTGGAAATGATGTCTGTGCTGCCACTGTCTCTCTGTGGTAATAGCATCATCAATCATTTCACTTGTGA
AATTCTGGCCATCTTGAAATTGGTTTGTGTGGACACCTCCCTGGTGCAGTTAATCATGCTGGTGATCAGTGTACTTCTTC
TCCCCATGCCAATGCTACTCATTTGTATCTCTTATGCATTTATCCTCGCCAGTATCCTGAGAATCAGCTCAGTGGAAGGT
CGAAGTAAAGCCTTTTCAACGTGCACAGCCCACCTGATGGTGGTAGTTTTGTTCTATGGGACGGCTCTCTCCATGCACCT
GAAGCCCTCCGCTGTAGATTCACAGGAAATAGACAAATTTATGGCTTTGGTGTATGCCGGACAAACCCCCATGTTGAATC
CTATCATCTATAGTCTACGGAACAAAGAGGTGAAAGTGGCCTTGAAAAAATTGCTGATTAGAAATCATTTTAATACTGCC
TTCATTTCCATCCTCAAATAACAATCACACTCATATAGA

TABLE L2
CG93817-01 Protein Sequence

Start: 22 ORF Stop: 979 Frame: 1
>CG93817-01-prot 319 aa

(SEQ ID NO:638)

MFPANWTSVKVFFFLGFFHYPKVQVIIFAVCLLMYLITLLGNIFLISITI
LDSHLHTPMYLFLSNLSFLDIWYSSSALSPMLANFVSGRVTISFSGCATQ
MYLSLAMGSTECVLLPMMAYDRYVAICNPLRYPVIMNRRTCVQIAAGSWM
TGCLTAMVEMMSVLPLSLCGNSIINHFTCEILAILKLVCVDTSLVQLIML
VISVLLLPMPMLLICISYAFILASILRISSVEGRSKAFSTCTAHLMVVVL
FYGTALSMHLKPSAVDSQEIDKFMALVYAGQTPMLNPIIYSLRNKEVKVA
LKKLLIRNHFNTAFISILK

L3. RTQ-PCR.

Quantitative expression analysis of clones in various cells and tissues were performed as described in Example C. Expression of gene CG93817-01 was assessed using the primer-probe set Ag1653, described in Tables LAB and. Results of the RTQ-PCR runs are shown in Tables LAC, LAD, LAE and LAF.

TABLE LAB

Probe Name: Ag1653

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-TCTCCTTTCTGGACATCTGGTA-3′ | 58.8 | 22 | 218 | 639 |
| Probe | TET-5′-TCCAATGCTGGCAAACTTTGTTTCAG-3′-TAMRA | 69 | 26 | 258 | 640 |
| Reverse | 5′-GCACCCTGAGAATGAAATAGTG-3′ | 58.7 | 22 | 291 | 641 |

TABLE LAC
Panel 1.3D

| | Rel. Expr., % |
|---|---|
| Tissue Name | 1.3dx4tm5594t_ag1653_b2 |
| Adipose | 23.3 |
| Adrenal gland | 0 |
| Bladder | 0 |
| Bone marrow | 0 |
| Brain (amygdala) | 0 |
| Brain (cerebellum) | 0 |
| Brain (fetal) | 0 |
| Brain (hippocampus) | 0 |
| Cerebral Cortex | 0 |
| Brain (substantia nigra) | 0 |
| Brain (thalamus) | 0 |
| Brain (whole) | 0 |
| Colorectal | 0 |
| Heart (fetal) | 0 |
| Liver adenocarcinoma | 0 |
| Heart | 0 |
| Kidney | 0 |
| Kidney (fetal) | 0 |
| Liver | 0 |
| Liver (fetal) | 0 |
| Lung | 0 |
| Lung (fetal) | 0 |
| Lymph node | 0 |
| Mammary gland | 0 |
| Fetal Skeletal | 0 |
| Ovary | 0 |
| Pancreas | 0 |
| Pituitary gland | 0 |
| Placenta | 0 |
| Prostate | 0 |
| Salivary gland | 0 |
| Skeletal muscle | 0 |
| Small intestine | 0 |
| Spinal cord | 0 |
| Spleen | 0.2 |
| Stomach | 0 |
| Testis | 0 |
| Thymus | 0 |
| Thyroid | 0 |
| Trachea | 0 |
| Uterus | 0 |
| genomic DNA control | 18.9 |
| Chemistry Control | 100 |

Panel 2.2. The data generated using panel 2.2 in sub-optimal. During the attempt to normalize the panel, some samples were diluted too much. It was also established that the quality of some of the RNAs was sub-optimal. This result in too many false negative and a reduce delta between diseased and normal tissues. The suggestion is to use this data to prioritize further analysis with TaqMan.

TABLE LAD
Panel 2.2

| | Rel. Expr., % |
|---|---|
| Tissue Name | 2.2x4tm6363t_ag1653_a1 |
| Normal Colon GENPAK 061003 | 0.0 |
| 97759 Colon cancer (OD06064) | 0.0 |
| 97760 Colon cancer NAT (OD06064) | 0.0 |
| 97778 Colon cancer (OD06159) | 0.0 |
| 97779 Colon cancer NAT (OD06159) | 0.0 |
| 98861 Colon cancer (OD06297-04) | 0.0 |
| 98862 Colon cancer NAT (OD06297-015) | 0.0 |
| 83237 CC Gr.2 ascend colon (ODO3921) | 0.0 |
| 83238 CC NAT (ODO3921) | 0.0 |
| 97766 Colon cancer metastasis (OD06104) | 0.0 |
| 97767 Lung NAT (OD06104) | 0.0 |
| 87472 Colon mets to lung (OD04451-01) | 0.0 |
| 87473 Lung NAT (OD04451-02) | 0.0 |
| Normal Prostate Clontech A+ 6546-1 | 0.0 |
| (8090438) | |
| 84140 Prostate Cancer (OD04410) | 0.0 |
| 84141 Prostate NAT (OD04410) | 0.0 |
| Normal Ovary Res. Gen. | 0.0 |
| 98863 Ovarian cancer (OD06283-03) | 0.0 |
| 98865 Ovarian cancer NAT/fallopian tube | 0.0 |
| (OD06283-07) | |
| Ovarian Cancer GENPAK 064008 | 0.0 |
| 97773 Ovarian cancer (OD06145) | 0.0 |
| 97775 Ovarian cancer NAT (OD06145) | 16.0 |
| 98853 Ovarian cancer (OD06455-03) | 0.0 |
| 98854 Ovarian NAT (OD06455-07) | 0.0 |
| Fallopian tube | |
| Normal Lung GENPAK 061010 | 0.0 |
| 92337 Invasive poor diff. lung adeno | 30.5 |
| (ODO4945-01 | |
| 92338 Lung NAT (ODO4945-03) | 0.0 |
| 84136 Lung Malignant Cancer (OD03126) | 0.0 |
| 84137 Lung NAT (OD03126) | 0.0 |
| 90372 Lung Cancer (OD05014A) | 0.0 |
| 90373 Lung NAT (OD05014B) | 0.0 |
| 97761 Lung cancer (OD06081) | 0.0 |
| 97762 Lung cancer NAT (OD06081) | 0.0 |
| 85950 Lung Cancer (OD04237-01) | 6.8 |
| 85970 Lung NAT (OD04237-02) | 6.7 |
| 83255 Ocular Mel Met to Liver (ODO4310) | 0.0 |
| 83256 Liver NAT (ODO4310) | 0.0 |
| 84139 Melanoma Mets to Lung (OD04321) | 0.0 |
| 84138 Lung NAT (OD04321) | 0.0 |
| Normal Kidney GENPAK 061008 | 0.0 |
| 83786 Kidney Ca, Nuclear grade 2 | 0.0 |
| (OD04338) | |
| 83787 Kidney NAT (OD04338) | 0.0 |
| 83788 Kidney Ca Nuclear grade 1/2 | 12.8 |
| (OD04339) | |
| 83789 Kidney NAT OD04339) | 0.0 |
| 83790 Kidney Ca, Clear cell type | 0.0 |
| (OD04340) | |
| 83791 Kidney NAT (OD04340) | 0.0 |
| 83792 Kidney Ca, Nuclear grade 3 | 7.8 |
| (OD04348) | |
| 83793 Kidney NAT (OD04348) | 0.0 |
| 98938 Kidney malignant cancer | 0.0 |
| (OD06204B) | |
| 98939 Kidney normal adjacent tissue | 0.0 |
| (OD06204E) | |
| 85973 Kidney Cancer (OD04450-01) | 0.0 |
| 85974 Kidney NAT (OD04450-03) | 0.0 |
| Kidney Cancer Clontech 8120613 | 0.0 |
| Kidney NAT Clontech 8120614 | 0.0 |
| Kidney Cancer Clontech 9010320 | 0.0 |
| Kidney NAT Clontech 9010321 | 0.0 |
| Kidney Cancer Clontech 8120607 | 0.0 |
| Kidney NAT Clontech 8120608 | 0.0 |
| Normal Uterus GENPAK 061018 | 0.0 |
| Uterus Cancer GENPAK 064011 | 0.0 |
| Normal Thyroid Clontech A+ 6570-1 | 0.0 |
| (7080817) | |
| Thyroid Cancer GENPAK 064010 | 0.0 |
| Thyroid Cancer INVITROGEN A302152 | 0.0 |
| Thyroid NAT INVITROGEN A302153 | 100.0 |
| Normal Breast GENPAK 061019 | 0.0 |
| 84877 Breast Cancer (OD04566) | 0.0 |
| Breast Cancer Res. Gen. 1024 | 0.0 |
| 85975 Breast Cancer (OD04590-01) | 0.0 |
| 85976 Breast Cancer Mets (OD04590-03) | 0.0 |
| 87070 Breast Cancer Metastasis | 0.0 |
| (OD04655-05) | |
| GENPAK Breast Cancer 064006 | 0.0 |
| Breast Cancer Clontech 9100266 | 0.0 |
| Breast NAT Clontech 9100265 | 0.0 |
| Breast Cancer INVITROGEN A209073 | 0.0 |
| Breast NAT INVITROGEN A2090734 | 0.0 |
| 97763 Breast cancer (OD06083) | 0.0 |
| 97764 Breast cancer node metastasis | 19.7 |
| (OD06083) | |
| Normal Liver GENPAK 061009 | 48.8 |
| Liver Cancer Research Genetics RNA 1026 | 0.0 |
| Liver Cancer Research Genetics RNA 1025 | 0.0 |
| Paired Liver Cancer Tissue Research | 0.0 |
| Genetics RNA 6004-T | |
| Paired Liver Tissue Research Genetics | 0.0 |
| RNA 6004-N | |
| Paired Liver Cancer Tissue Research | 0.0 |
| Genetics RNA 6005-T | |
| Paired Liver Tissue Research Genetics | 0.0 |
| RNA 6005-N | |
| Liver Cancer GENPAK 064003 | 0.0 |
| Normal Bladder GENPAK 061001 | 0.0 |
| Bladder Cancer Research Genetics RNA | 0.0 |
| 1023 | |
| Bladder Cancer INVITROGEN A302173 | 0.0 |
| Normal Stomach GENPAK 061017 | 0.0 |
| Gastric Cancer Clontech 9060397 | 0.0 |
| NAT Stomach Clontech 9060396 | 0.0 |
| Gastric Cancer Clontech 9060395 | 0.0 |
| NAT Stomach Clontech 9060394 | 0.0 |
| Gastric Cancer GENPAK 064005 | 0.0 |

TABLE LAE
Panel 4D

| Tissue Name | Rel. Expr., |
|---|---|
| Liver cirrhosis | −100% (CT=32.7) |

TABLE LAF
Panel 5D

| | Rel. Expr., % |
|---|---|
| Tissue Name | 5dtm5883t_ag1653_s1 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 97481_Patient-08sk_skeletal muscle | 0.0 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 0.0 |
| 97501_Patient-12sk_skeletal muscle | 0.0 |
| 97502_Patient-12ut_uterus | 0.0 |
| 97503_Patient-12pl_placenta | 0.0 |
| 94721_Donor 2 U - A_Mesenchymal Stem | 0.0 |
| Cells | |
| 94722_Donor 2 U - B_Mesenchymal Stem | 0.0 |
| Cells | |
| 94723_Donor 2 U - C_Mesenchymal Stem | 0.0 |
| Cells | |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem | 0.0 |
| Cells | |
| 94743_Donor 3 U - B_Mesenchymal Stem | 0.0 |
| Cells | |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 0.0 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |
| genomic_DNA | 63.7 |
| Chemistry Control | 100.0 |

M. NOV32a—Human HMG-COA LYASE Precursor-Like Protein

Discovery Process: The following sections describe the study design(s) and the techniques used to identify the HMG-COA LYASE-encoded protein and any variants, thereof, as being suitable as diagnostic markers, targets for an antibody therapeutic and targets for a small molecule drugs for Obesity and Diabetes.

Studies: MB.01 Insulin Resistance

Study Statements: The spontaneously hypertensive rat (SHR) is a strain exhibiting features of the human Metabolic Syndrome X. The phenotypic features include obesity, hyperglycemia, hypertension, dyslipidemia and dysfibrinolysis. Tissues were removed from adult male rats and a control strain (Wistar-Kyoto) to identify the gene expression differences that underlie the pathologic state in the SHR and in animals treated with various anti-hyperglycemic agents such as troglitizone. Tissues included sub-cutaneous adipose, visceral adipose and liver.

Species #1 rat—Strains—WKY, SHR, treatment with 0.02% DMSO, treatment with Troglitazone LD10

HMG-COA LYASE: 3-Hydroxy-3-methylglutaryl coenzyme A lyase (HL) catalyzes the final step of ketogenesis, an important pathway of mammalian energy metabolism. HL deficiency known as hydroxymethylglutaricaciduria is an autosomal recessive inborn error in man leading to episodes of hypoglycemia and coma.

SPECIES #1 rat (WKY strain treated with Troglitazone LD10 vs. 0.02% DMSO)

A gene fragment of the rat HMG-COA LYASE was initially found to be upregulated by 1.6 fold in the liver of WKY rats treated with Troglitazone LD10 relative to WKY rats treated with 0.02% DMSO as control using CuraGen's GeneCalling™ method of differential gene expression. A differentially expressed rat gene fragment migrating at approximately 426.4 nucleotides in length (Table M2A.—vertical line) was definitively identified as a component of the rat HMG-COA LYASE cDNA in the Troglitazone treated and the untreated WKY control rats (in the graphs, the abscissa is measured in lengths of nucleotides and the ordinate is measured as signal response). The method of competitive PCR was used for conformation of the gene assessment. The chromatographic peaks corresponding to the gene fragment of the rat HMG-COA LYASE are ablated when a gene-specific primer (see below) which competes with primers in the linker-adaptors during the PCR amplification. The peaks at 426.4 nt in length are ablated in the sample from both the Troglitazone treated and the untreated WKY control rats. The altered expression in of these genes in the animal model support the role of HMG-COA LYASE in the pathogenesis of obesity and/or diabetes.

SPECIES #1 rat (SHR strain treated with Troglitazone LD10 vs. 0.02% DMSO)

A gene fragment of the rat HMG-COA LYASE was initially found to be upregulated by 2.6 fold in the liver of SHR rats treated with Troglitazone LD10 relative to SHR rats treated with 0.02% DMSO as control using CuraGen's GeneCalling™ method of differential gene expression. A differentially expressed rat gene fragment migrating at approximately 48.2 nucleotides in length (Table M2A—vertical line) was definitively identified as a component of the rat HMG-COA LYASE cDNA in the Troglitazone treated and the untreated SHR control rats (in the graphs, the abscissa is measured in lengths of nucleotides and the ordinate is measured as signal response). The method of competitive PCR was used for conformation of the gene assessment. The chromatographic peaks corresponding to the gene fragment of the rat HMG-COA LYASE are ablated when a gene-specific primer (see below) which competes with primers in the linker-adaptors during the PCR amplification. The peaks at 48.2 nt in length are ablated in the sample from both the Troglitazone treated and the untreated WKY control rats. The altered expression in of these genes in the animal model support the role of HMG-COA LYASE in the pathogenesis of obesity and/or diabetes.

TABLE M1
Partial rat HMG-COA LYASE Gene Sequence

Gene Sequence identified in WKY Troglitazone LD10 vs. 0.02% DMSO
(Identified fragment from 612 to 1038 in bold, band size: 427)

(SEQ ID NO:642)

GGCCCCCGAG ATGGTCTGCA GAATGAAAAG AGTATCGTGC CGACGCCAGT GAAAATCAAA CTGATAGACA
TGCTATCCGA AGCAGGGCTC CCGGTCATCG AGGCCACCAG CTTTGTCTCT CCCAAGTGGG TGCCGCAGAT
GGCTGACCAC TCTGACGTCT TGAAGGGCAT TCAGAAGTTT CCCGGCATCA ACTACCCGGT CCTGACACCA
AACATGAAAG GCTTTGAGGA AGCGGTAGCT GCAGGTGCCA AGGAAGTGAG CATCTTTGGG GCTGCGTCCG
AGCTCTTCAC CCGGAAGAAT GTGAACTGCT CTATAGAGGA GAGTTTCCAG CGCTTTGATG GGGTCATGCA
GGCCGCGAGG GCTGCCAGCA TCTCTGTGAG AGGGTATGTC TCCTGTGCCC TCGGATGTCC CTACGAGGGG
AAGGTCTCCC CGGCTAAAGT TGCTGAGGTC GCCAAGAAGT TGTACTCAAT GGGCTGCTAT GAGATCTCCC
TTGGGGACAC CATTGGCGTA GGCACGCCAG GACTCATGAA AGACATGCTG ACTGCTGTCC TGCATGAAGT
GCCTGTGGCC GCATTGGCTG TCCACTGCCA TGACACCTAT GGCCAAGCTC TGGCCAACAC GTTGGTGGCC
CTGCAGATGG GAGTGAGCGT TGTGGACTCC TCGGTGGCAG GACTCGGAGG CTGTCCCTAT GCAAAGGGGG
CGTCAGGAAA CTTGGGTACC GAGGACCTGG TCTACATGCT GACTGGCTTA GGGATTCACA CGGGTGTGAA
CCTCCAGAAG CTCCTAGAAG CCGGGGACTT CATCTGTCAA GCCCTGAACA GAAAAACCAG TTCCAAAGTG
GCACAGGCCA CCTGCAAACT CTGAGCCCCT TGTTCACCTA AACCGGAACT GTGGGAGTTG GGTGTACACA
ATGATTCCTG GATGGGGAAA TGGAATGAAG GCAAATGAGC CGGCCTCACA GAGGTCCCTC TCCTACATAG
AAGGGCTAGA GCTGCCAGCA CGCCCGGACC AGCTCCCCAG AGCTGCGTGC CTAAGCACTG CTTGGCTGGC
CCTGGGTGAG TCCACTAGCC AGCAGAGCTG ACATCCATGT GCCACGACCG CGGGTCCCAT GTTCTACCTC
TGAGGACAGC AGCGCCTTTG CTGAAATGGT GGGCTCAATC TACTGCGGTG GCCGACTGCC AACTCCAGCG
TCTCTGGGAA ATCTCTGTAC GTGATTCTTG AAAACAGCTT ATGTAATTAA AGGTTTAATT TTCTAATATC

Table M2A shows the differential regulation of HMG-CoA lyase by the differentially expressed rat HMB-CoA Lyase gene fragment from Discovery Study MB.01 identified in WKY Troglitazone LD10 vs. 0.02% DMSO.

[image]

[image]

TABLE M3
Partial rat HMG-COA LYASE Gene Sequence

Gene Sequence identified in SHR Troglitazone LD10 vs. 0.02% DMSO
(Identified fragment from 612 to 659 in bold. band size: 48)

(SEQ ID NO:643)

GGCCCCCGAGATGGTCTGCAGAATGAAAAGAGTATCGTGCCGACGCCAGTGAAAATCAAACTGATAGACATGCTATCCGA
AGCAGGGCTCCCGGTCATCGAGGCCACCAGCTTTGTCTCTCCCAAGTGGGTGCCGCAGATGGCTGACCACTCTGACGTCT
TGAAGGGCATTCAGAAGTTTCCCGGCATCAACTACCCGGTCCTGACACCAAACATGAAAGGCTTTGAGGAAGCGGTAGCT
GCAGGTGCCAAGGAAGTGAGCATCTTTGGGGCTGCGTCCGAGCTCTTCACCCGGAAGAATGTGAACTGCTCTATAGAGGA
GAGTTTCCAGCGCTTTGATGGGGTCATGCAGGCCGCGAGGGCTGCCAGCATCTCTGTGACAGGGTATGTCTCCTGTGCCC
TCGGATGTCCCTACGAGGGGAAGGTCTCCCCGGCTAAAGTTGCTGAGGTCGCCAAGAAGTTGTACTCAATGGGCTGCTAT
GAGATCTCCCTTGGGGACACCATTGGCGTAGGCACGCCAGGACTCATGAAAGACATGCTGACTGCTGTCCTGCATGAAGT
GCCTGTGGCCGCATTGGCTGTCCACTGCCATGACACCTATGGCCAAGCTCTGGCCAACACGTTGGTGGCCCTGCAGATGG
GAGTGAGCGTTGTGGACTCCTCGGTGGCAGGACTCGGAGGCTGTCCCTATGCAAAGGGGGCGTCAGGAAACTTGGCTACC
GAGGACCTGGTCTACATGCTGACTGGCTTAGGGATTCACACGGGTGTGAACCTCCAGAAGCTCCTAGAAGCCGGGGACTT
CATCTGTCAAGCCCTGAACAGAAAAACCAGTTCCAAAGTGGCACAGGCCACCTGCAAACTCTGAGCCCCTTGTTCACCTA
AACCGGAACTGTGGGAGTTGGGTGTACACAATGATTCCTGGATGGGGAAATGGAATGAAGGCAAATGAGCCGGCCTCACA
GAGGTCCCTCTCCTACATAGAAGGGCTAGAGCTGCCAGCACGCCCGGAC

The sequence of Acc. No CG96859-03 was derived by laboratory screening of cDNA library by the two-hybrid approach. cDNA fragments covering either the full length of the DNA sequence, or part of the sequence, or both, were sequenced. In silico prediction was based on sequences available in CuraGen Corporation's proprietary sequence databases or in the public human sequence databases, and provided either the full-length DNA sequence, or some portion thereof.

TABLE M4
Human HMG-CoA Lyase-like CG96859-03 DNA and Protein Sequence

(SEQ ID NO:644)

ATGCCCCTTATTTCCACATCATCCCCAGGCCTTCAGGTGGGCAAGGGGCTCAGAGTTTACAGGTAGCCTGAGCCACTTTG
GAGCTAGTTTTTCTGTTCAGGGCTTGACAGATAAAGTTTCCAGCTTCCAGAAGCTTCTGGAGATTCACACCCGTGTGAAT
GCCCAAGCCCTCTAGCATGTAGACCAGGTCTTCTGTGGCCAAGTTTCCTGATGCCCCCTGTGCGTAGGGACAGCCTCCAA
GTCCTGCCACAGAAGAGTCCACGACACTCACTCCCATCTGCAGGGCCATCAAGGTGTTGGCCAGGGCTTGACCATAGGTG
TCATGGCAGTGGACAGCCAGGGCAGCCAGAGGCACTTCCTGCATGACAGCAGACAGCATGTCTTTCATGATCCCTGGGGT
GCCCACACCAATGGTGTCCCCCAGGGAGATCTCGTAGCAGCCCATTGAGTAGAACTTCTTGGTGACCTCAGCTACTTTAG
CTGGGGAGATCTTCCCTTCATAAGGGCAGCCAAGAGCACAGGAGACGTACCCCCGCACAGAAATATTGGCTGACTGCGCT
GCCTTCAGGATTGCGTCAAACCTCTGAAAACTCTCCTCTATGGAACAATTGATGTTCTTCTTGGTGAAGAGCTCTGAGGC
AGCTCCAAAGATGACTACTTCCTTGGCTCCAGCAGCAACCGCTGCCTCGAAGCCTTTCAAATTTGGGGTCAGGACTGGGT
AGTTGATGCCAGGAAACTTCTGAATGCCCTTCAAGACTTCAGTGTGGTCACCCATCTGGGGAACCCACTTAGGAGACACA
AAGCTGGTGGTTTCTATAACAGAGAGTCCTGCTTCAGAAAGCATGTCTATCAGCTTGATTTTCACTGGAGTAGATACGAT
ATTCTTTTCATTTTGTAGTCCATCTCGGGCACCAACTTCCACAATTTTCACCCGCTTTGGTAAAGTGCCCATAGATGAGG
TGCTGACAGCCCGGAGGGACGCCAAGCCCACCAGTCGCCGCGGAAGCGCCTTCCTCATTGCTGCCATCTTGGCCGGAATT
T

TABLE M5
>CG96859-03 Protein

325 aa

(SEQ ID NO:645)

MAAMRKALPRRLVGLASLRAVSTSSMGTLPKRVKIVEVGPRDGLQNEKNI
VSTPVKIKLIDMLSEAGLSVIETTSFVSPKWVPQMGDHTEVLKGIQKFPG
INYPVLTPNLKGFEAAVAAGAKEVVIFGAASELFTKKNINCSIEESFQRF
DAILKAAQSANISVRGYVSCALGCPYEGKISPAKVAEVTKKFYSMGCYEI
SLGDTIGVGTPGIMKDMLSAVMQEVPLAALAVHCHDTYGQALANTLMALQ
MGVSVVDSSVAGLGGCPYAQGASGNLATEDLVYMLEGLGIHTGVNLQKLL
EAGNFICQALNRKTSSKVAQATCKL

The following is an alignment of the protein sequences of CG96859-03, another public form of HMG CoA lyase with one aa difference (P35914), a novel splice form of HMG CoA lyase (CG96859-02), and the rat and mouse orthologues of HMG-COA LYASE.

[image]

[image]

In addition to the human version of the HMG-COA LYASE identified as being differentially expressed in the experimental study, other variants have been identified by direct sequencing of cDNAs derived from many different human tissues and from sequences in public databases. Two splice-form variants have been identified at CuraGen. Below is a clustalW of the CG96859-03 (SEQ ID NO:651) and the two alternative spliced forms, CG96859-02 (SEQ ID NO:652) and CG96859-05 (SEQ IDNO:653). No amino acid-changing cSNPs were identified. The preferred variant of all those identified, to be used for screening purposes, is CG96859-03.

Expression Profiles: Hydroxymethylglutaryl-CoA Lyase CG96859-03 Expression

Quantitative expression analysis of clones in various cells and tissues was performed as described in Example C. Expression of gene CG96859-02 was assessed using the primer-probe set Ag4735, described in Table MAA. Results of the RTQ-PCR runs are shown in Tables MAB and MC. This primer set recognizes all three isoforms of HMG-CoA lyase (CG96859-02/03/05).

TABLE MAA

Probe Name Ag4735

| | | | | |
|---|---|---|---|---|
| | | | Start | SEQ ID |
| Primers | Sequences | Length | Position | NO |

| | | | | |
|---|---|---|---|---|
| Forward | 5′-tgaccgctgcctcga-3′ | 15 | 990 | 654 |
| Probe | TET-5′-atgccaggaaacttctgaatgccc-3′-TAMRA | 24 | 1040 | 655 |
| Reverse | 5′-gtgtctcctaagtgggttcc-3′ | 20 | 1094 | 656 |

TABLE MAB
General_screening_panel_v1.4

| | Rel. Exp.(%) |
|---|---|
| | Ag4735, Run |
| Tissue Name | 222262773 |
| Adipose | 3.1 |
| Melanoma* Hs688(A).T | 23.2 |
| Melanoma* Hs688(B).T | 18.7 |
| Melanoma* M14 | 19.2 |
| Melanoma* LOXIMVI | 4.5 |
| Melanoma* SK-MEL-5 | 13.1 |
| Squamous cell carcinoma SCC-4 | 7.9 |
| Testis Pool | 5.1 |
| Prostate ca.* (bone met) PC-3 | 31.6 |
| Prostate Pool | 3.4 |
| Placenta | 5.8 |
| Uterus Pool | 1.2 |
| Ovarian ca. OVCAR-3 | 15.0 |
| Ovarian ca. SK-OV-3 | 26.2 |
| Ovarian ca. OVCAR-4 | 17.2 |
| Ovarian ca. OVCAR-5 | 35.1 |
| Ovarian ca. IGROV-1 | 20.0 |
| Ovarian ca. OVCAR-8 | 15.5 |
| Ovary | 4.7 |
| Breast ca. MCF-7 | 35.4 |
| Breast ca. MDA-MB-231 | 20.0 |
| Breast ca. BT 549 | 44.4 |
| Breast ca. T47D | 100.0 |
| Breast ca. MDA-N | 2.9 |
| Breast Pool | 5.7 |
| Trachea | 8.6 |
| Lung | 2.7 |
| Fetal Lung | 11.8 |
| Lung ca. NCI-N417 | 4.4 |
| Lung ca. LX-1 | 24.0 |
| Lung ca. NCI-H146 | 2.0 |
| Lung ca. SHP-77 | 8.7 |
| Lung ca. A549 | 12.9 |
| Lung ca. NCI-H526 | 4.5 |
| Lung ca. NCI-H23 | 18.3 |
| Lung ca. NCI-H460 | 11.0 |
| Lung ca. HOP-62 | 15.8 |
| Lung ca. NCI-H522 | 27.4 |
| Liver | 17.0 |
| Fetal Liver | 26.1 |
| Liver ca. HepG2 | 41.5 |
| Kidney Pool | 12.5 |
| Fetal Kidney | 7.7 |
| Renal ca. 786-0 | 29.7 |
| Renal ca. A498 | 5.5 |
| Renal ca. ACHN | 19.1 |
| Renal ca. UO-31 | 7.5 |
| Renal ca. TK-10 | 34.9 |
| Bladder | 11.4 |
| Gastric ca. (liver met.) NCI-N87 | 51.4 |
| Gastric ca. KATO III | 42.6 |
| Colon ca. SW-948 | 9.2 |
| Colon ca. SW480 | 19.9 |
| Colon ca.* (SW480 met) SW620 | 20.3 |
| Colon ca. HT29 | 20.2 |
| Colon ca. HCT-116 | 25.0 |
| Colon ca. CaCo-2 | 18.7 |
| Colon cancer tissue | 12.0 |
| Colon ca. SW1116 | 8.4 |
| Colon ca. Colo-205 | 10.3 |
| Colon ca. SW-48 | 6.5 |
| Colon Pool | 6.2 |
| Small Intestine Pool | 4.7 |
| Stomach Pool | 3.6 |
| Bone Marrow Pool | 2.1 |
| Fetal Heart | 4.0 |
| Heart Pool | 6.2 |
| Lymph Node Pool | 6.1 |
| Fetal Skeletal Muscle | 4.5 |
| Skeletal Muscle Pool | 19.9 |
| Spleen Pool | 5.1 |
| Thymus Pool | 5.2 |
| CNS cancer (glio/astro) U87-MG | 42.3 |
| CNS cancer (glio/astro) U-118-MG | 25.5 |
| CNS cancer (neuro;met) SK-N-AS | 10.9 |
| CNS cancer (astro) SF-539 | 23.8 |
| CNS cancer (astro) SNB-75 | 63.3 |
| CNS cancer (glio) SNB-19 | 23.2 |
| CNS cancer (glio) SF-295 | 41.5 |
| Brain (Amygdala) Pool | 7.0 |
| Brain (cerebellum) | 8.5 |
| Brain (fetal) | 5.0 |
| Brain (Hippocampus) Pool | 9.5 |
| Cerebral Cortex Pool | 7.3 |
| Brain (Substantia nigra) Pool | 10.0 |
| Brain (Thalamus) Pool | 12.1 |
| Brain (whole) | 7.5 |
| Spinal Cord Pool | 15.8 |
| Adrenal Gland | 12.3 |
| Pituitary gland Pool | 2.5 |
| Salivary Gland | 5.1 |
| Thyroid (female) | 10.2 |
| Pancreatic ca. CAPAN2 | 30.4 |
| Pancreas Pool | 8.0 |

TABLE MAC
Panel 5D

| | Rel. Exp.(%) |
|---|---|
| | Ag4735, Run |
| Tissue Name | 204263058 |
| 97457_Patient-02go_adipose | 12.0 |
| 97476_Patient-07sk_skeletal muscle | 9.8 |
| 97477_Patient-07ut_uterus | 12.4 |
| 97478_Patient-07pl_placenta | 17.8 |
| 97481_Patient-08sk_skeletal muscle | 5.6 |
| 97482_Patient-08ut_uterus | 11.6 |
| 97483_Patient-08pl_placenta | 8.8 |
| 97486_Patient-09sk_skeletal muscle | 5.4 |
| 97487_Patient-09ut_uterus | 10.8 |
| 97488_Patient-09pl_placenta | 10.8 |
| 97492_Patient-10ut_uterus | 7.3 |
| 97493_Patient-10pl_placenta | 40.9 |
| 97495_Patient-11go_adipose | 11.9 |
| 97496_Patient-11sk_skeletal muscle | 41.2 |
| 97497_Patient-11ut_uterus | 20.2 |
| 97498_Patient-11pl_placenta | 17.6 |
| 97500_Patient-12go_adipose | 15.6 |
| 97501_Patient-12sk_skeletal muscle | 88.9 |
| 97502_Patient-12ut_uterus | 19.6 |
| 97503_Patient-12pl_placenta | 18.2 |
| 94721_Donor 2 U - | 22.7 |
| A_Mesenchymal Stem Cells | |
| 94722_Donor 2 U - | 18.4 |
| B_Mesenchymal Stem Cells | |
| 94723_Donor 2 U - | 16.4 |
| C_Mesenchymal Stem Cells | |
| 94709_Donor 2 AM - A_adipose | 37.1 |
| 94710_Donor 2 AM - B_adipose | 14.9 |
| 94711_Donor 2 AM - C_adipose | 12.0 |
| 94712_Donor 2 AD - A_adipose | 36.1 |
| 94713_Donor 2 AD - B_adipose | 40.3 |
| 94714_Donor 2 AD - C_adipose | 24.8 |
| 94742_Donor 3 U - A_Mesenchymal | 18.8 |
| Stem Cells | |
| 94743_Donor 3 U - B_Mesenchymal | 18.7 |
| Stem Cells | |
| 94730_Donor 3 AM - A_adipose | 16.0 |
| 94731_Donor 3 AM - B_adipose | 16.2 |
| 94732_Donor 3 AM - C_adipose | 11.4 |
| 94733_Donor 3 AD - A_adipose | 46.0 |
| 94734_Donor 3 AD - B_adipose | 19.9 |
| 94735_Donor 3 AD - C_adipose | 19.5 |
| 77138_Liver_HepG2untreated | 100.0 |
| 73556_Heart_Cardiac stromal cells | 4.3 |
| (primary) | |
| 81735_Small Intestine | 23.5 |
| 72409_Kidney_Proximal | 11.8 |
| Convoluted Tubule | |
| 82685_Small intestine_Duodenum | 27.4 |
| 90650_Adrenal_Adrenocortical | 8.2 |
| adenoma | |
| 72410_Kidney_HRCE | 57.4 |
| 72411_Kidney_HRE | 25.2 |
| 73139_Uterus_Uterine smooth | 12.5 |
| muscle cells | |

General_screening_panel_v1.4 Summary: The primer set Ag4735 recognizes all three isoforms of the HMG-CoA lyase.

Panel 5D Summary: The primer set Ag4735 recognizes all three isoforms of the HMG-CoA lyase.

ZA. CG96859-03 Hydroxymethylglutaryl-CoA Lyase-Like Protein

Expression of gene CG96859-01 was assessed using the primer-probe set Ag4736, described in Table ZAA. Results of the RTQ-PCR runs are shown in Tables ZAB and ZAC. The primer set Ag4736 was developed to recognize only the CG96859-03 splice variant of the gene.

TABLE ZAA

Probe Name Ag4736

| | | | | |
|---|---|---|---|---|
| | | | Start | SEQ ID |
| Primers | Sequences | Length | Position | NO |

| | | | | |
|---|---|---|---|---|
| Forward | 5′-aagtagctgaggtcaccaaga-3′ | 21 | 565 | 657 |
| Probe | TET-5′-ttctactcaatgggctgctacgagatctcc-3′-TAMRA | 30 | 588 | 658 |
| Reverse | 5′-tagcatgtctttcatgatcc-3′ | 20 | 649 | 659 |

TABLE ZAB
General_screening_panel_v1.5

| | Rel. Exp.(%) |
|---|---|
| | Ag4736, Run |
| Tissue Name | 228714901 |
| Adipose | 2.7 |
| Melanoma* Hs688(A).T | 0.0 |
| Melanoma* Hs688(B).T | 0.0 |
| Melanoma* M14 | 10.7 |
| Melanoma* LOXIMVI | 0.6 |
| Melanoma* SK-MEL-5 | 100.0 |
| Squamous cell carcinoma SCC-4 | 5.1 |
| Testis Pool | 1.8 |
| Prostate ca.* (bone met) PC-3 | 0.0 |
| Prostate Pool | 0.9 |
| Placenta | 0.2 |
| Uterus Pool | 2.4 |
| Ovarian ca. OVCAR-3 | 6.2 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.9 |
| Ovarian ca. OVCAR-8 | 11.7 |
| Ovary | 1.5 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 1.2 |
| Trachea | 3.1 |
| Lung | 7.4 |
| Fetal Lung | 7.4 |
| Lung ca. NCI-N417 | 3.2 |
| Lung ca. LX-1 | 0.0 |
| Lung ca. NCI-H146 | 0.3 |
| Lung ca. SHP-77 | 0.1 |
| Lung ca. A549 | 0.0 |
| Lung ca. NCI-H526 | 0.0 |
| Lung ca. NCI-H23 | 3.4 |
| Lung ca. NCI-H460 | 0.0 |
| Lung ca. HOP-62 | 1.7 |
| Lung ca. NCI-H522 | 0.2 |
| Liver | 0.0 |
| Fetal Liver | 0.2 |
| Liver ca. HepG2 | 0.0 |
| Kidney Pool | 3.7 |
| Fetal Kidney | 0.6 |
| Renal ca. 786-0 | 0.0 |
| Renal ca. A498 | 0.0 |
| Renal ca. ACHN | 0.6 |
| Renal ca. UO-31 | 0.1 |
| Renal ca. TK-10 | 0.0 |
| Bladder | 1.0 |
| Gastric ca. (liver met.) NCI-N87 | 0.8 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.4 |
| Colon cancer tissue | 1.2 |
| Colon ca. SW1116 | 0.0 |
| Colon ca. Colo-205 | 0.1 |
| Colon ca. SW-48 | 0.0 |
| Colon Pool | 0.4 |
| Small Intestine Pool | 0.9 |
| Stomach Pool | 0.9 |
| Bone Marrow Pool | 2.9 |
| Fetal Heart | 0.3 |
| Heart Pool | 0.6 |
| Lymph Node Pool | 0.7 |
| Fetal Skeletal Muscle | 3.4 |
| Skeletal Muscle Pool | 0.6 |
| Spleen Pool | 0.8 |
| Thymus Pool | 1.9 |
| CNS cancer (glio/astro) U87-MG | 0.4 |
| CNS cancer (glio/astro) U-118-MG | 7.7 |
| CNS cancer (neuro;met) SK-N-AS | 0.4 |
| CNS cancer (astro) SF-539 | 20.3 |
| CNS cancer (astro) SNB-75 | 5.8 |
| CNS cancer (glio) SNB-19 | 1.9 |
| CNS cancer (glio) SF-295 | 7.8 |
| Brain (Amygdala) Pool | 3.8 |
| Brain (cerebellum) | 15.5 |
| Brain (fetal) | 4.5 |
| Brain (Hippocampus) Pool | 7.3 |
| Cerebral Cortex Pool | 3.2 |
| Brain (Substantia nigra) Pool | 3.3 |
| Brain (Thalamus) Pool | 5.8 |
| Brain (whole) | 3.2 |
| Spinal Cord Pool | 14.4 |
| Adrenal Gland | 1.3 |
| Pituitary gland Pool | 2.4 |
| Salivary Gland | 0.2 |
| Thyroid (female) | 0.1 |
| Pancreatic ca. CAPAN2 | 0.0 |
| Pancreas Pool | 0.7 |

TABLE ZAC
Panel 5D

| | Rel. | | Rel. |
|---|---|---|---|
| | Exp. (%) | | Exp. (%) |
| | Ag4736, | | Ag4736, |
| | Run | | Run |
| Tissue Name | 204266936 | Tissue Name | 204266936 |
| 97457_Patient-02go_adipose | 0.0 | 94709_Donor 2 AM - A_adipose | 38.2 |
| 97476_Patient-07sk_skeletal | 3.3 | 94710_Donor 2 AM - B_adipose | 28.9 |
| muscle | | | |
| 97477_Patient-07ut_uterus | 7.6 | 94711_Donor 2 AM - C_adipose | 18.6 |
| 97478_Patient-07pl_placenta | 11.1 | 94712_Donor 2 AD - A_adipose | 50.3 |
| 97481_Patient-08sk_skeletal | 4.0 | 94713_Donor 2 AD - B_adipose | 65.1 |
| muscle | | | |
| 97482_Patient-08ut uterus | 7.3 | 94714_Donor 2 AD - C_adipose | 56.6 |
| 97483_Patient-08pl_placenta | 6.3 | 94742_Donor 3 U - A_Mesenchymal | 27.7 |
| | | Stem Cells | |
| 97486_Patient-09sk_skeletal | 3.9 | 94743_Donor 3 U - B_Mesenchymal | 20.2 |
| muscle | | Stem Cells | |
| 97487_Patient-09ut_uterus | 13.7 | 94730_Donor 3 AM - A_adipose | 29.9 |
| 97488_Patient-09pl_placenta | 27.0 | 94731_Donor 3 AM - B_adipose | 16.5 |
| 97492_Patient-10ut_uterus | 12.9 | 94732_Donor 3 AM - C_adipose | 14.3 |
| 97493_Patient-10pl_placenta | 44.4 | 94733_Donor 3 AD - A_adipose | 53.2 |
| 97495_Patient-11go_adipose | 13.9 | 94734_Donor 3 AD - B_adipose | 32.1 |
| 97496_Patient-11sk_skeletal | 21.0 | 94735_Donor 3 AD - C_adipose | 21.5 |
| muscle | | | |
| 97497_Patient-11ut_uterus | 38.4 | 77138_Liver_HepG2untreated | 100.0 |
| 97498_Patient-11pl_placenta | 24.8 | 73556_Heart_Cardiac stromal cells | 4.8 |
| | | (primary) | |
| 97500_Patient-12go_adipose | 29.5 | 81735_Small Intestine | 27.0 |
| 97501_Patient-12sk_skeletal | 82.4 | 72409_Kidney_Proximal | 11.0 |
| muscle | | Convoluted Tubule | |
| 97502_Patient-12ut_uterus | 28.3 | 82685_Small intestine_Duodenum | 26.4 |
| 97503_Patient-12pl_placenta | 27.0 | 90650_Adrenal_Adrenocortical | 6.3 |
| | | adenoma | |
| 94721_Donor 2 U - | 34.6 | 72410_Kidney_HRCE | 36.6 |
| A_Mesenchymal Stem Cells | | | |
| 94722_Donor 2 U - | 31.0 | 72411_Kidney_HRE | 6.7 |
| B_Mesenchymal Stem Cells | | | |
| 94723_Donor 2 U - | 35.8 | 73139_Uterus_Uterine smooth | 7.9 |
| C_Mesenchymal Stem Cells | | muscle cells | |

General_screening_panel_v1.5 Summary: Primer set Ag4736 is specific for alternative spliced variant CG96859-03.

Panel 5D Summary: Primer set Ag4736 is specific for alternative spliced variant CG96859-03.

Biochemistry and Cell Line Expression

The following illustrations summarize the biochemistry surrounding the human HMG-COA LYASE and potential assays that may be used to screen for antibody therapeutics or small molecule drugs to treat obesity and/or diabetes. Cell lines expressing the HMG-COA LYASE can be obtained from the RTQ-PCR results shown above. These and other HMG-COA LYASE expressing cell lines could be used for screening purposes.

HMG-CoA Lyase has the following catalytic activity:

3-hydroxy-3-methylglutaryl-CoA=acetyl-CoA+acetoacetate

HMG-CoA affects biochemical pathways relevant to the etiology and pathogenesis of obesity and/or diabetes. The scheme incorporates the unique findings of these discovery studies in conjunction with what has been reported in the literature. The outcome of inhibiting the action of the human Aryl Hydrocarbon Receptor would be a reduction of Insulin Resistance, a major problem in obesity and/or diabetes. HMG-CoA lyase uses HMG-CoA as a substrate to produce acetoacetate and acetyl-CoA. This is the final step in ketogenesis and leucine metabolism. Importantly, acetyl-CoA from this reaction can be fed back into the TCA cycle but also into lipogenic pathways.

Physical cDNA Clone Available for Expression and Screening Purposes

Exon linking and In-Frame cloning was performed as described above. Table M10 depicts the preferred cDNA(s) that encompass the coding portion of the human HMG-CoA lyase for expression of recombinant protein from any number of plasmid, phage or phagemid vectors in a variety of cellular systems for screening purposes. The corresponding amino acid sequence(s) are also listed. Although the sequences below are the preferred isoforms, any of the other isoforms may be used for similar purposes. Furthermore, under varying assay conditions, conditions may dictate that another isoform may supplant the listed isoforms. Table M11 shows the clustalw CG96859-03 and its working representatives CG96859-08 and -09 analysis. The working representatives of CG96859-03 are partials of CG96859-03. The CG96859-09 variant has an N-terminal Histidine tag used for protein purification.

TABLE M10
Physical cDNA Clone Available for Expression & Screening Purposes

>CG96859-08. 969 nt

(SEQ ID NO:660)

TCAGAGTTTACAGGTAGCCTGAGCCACTTTGGAGCTAGTTTTTCTGTTCAGGGCTTGACAGATAAAGTTTCCAGCTTCCA
GAAGCTTCTGGAGATTCACACCCGTGTGAATGCCCAAGCCCTCTAGCATGTAGACCAGGTCTTCTGTGGCCAAGTTTCCT
GATGCCCCCTGTGCGTAGGGACAGCCTCCAAGTCCTGCCACAGAAGAGTCCACGACACTCACTCCCATCTGCAGGGCCAT
CAAGGTGTTGGCCAGGGCTTGACCATAGGTGTCATGGCAGTGGACAGCCAGGGCAGCCAGAGGCACTTCCTGCATGACAG
CAGACAGCATGTCTTTCATGATCCCTGGGGTGCCCACACCAATGGTGTCCCCCAGGGAGATCTCGTAGCAGCCCATTGAG
TAGAACTTCTTGGTGACCTCAGCTACTTTAGCTGGGGAGATCTTCCCTTCATAAGGGCAGCCAAGAGCACAGGAGACGTA
CCCCCGCACAGAAATATTGGCTGACTGCGCTGCCTTCAGGATTGCGTCAAACCTCTGAAAACTCTCCTCTATGGAACAAT
TGATGTTCTTCTTGGTGAAGAGCTCTGAGGCAGCTCCAAAGATGACTACTTCCTTGGCTCCAGCAGCAACCGCTGCCTCG
AAGCCTTTCAAATTTGGGGTCAGGACTGGGTAGTTGATGCCAGGAAACTTCTGAATGCCCTTCAAGACTTCAGTGTGGTC
ACCCATCTGGGGAACCCACTTAGGAGACACAAAGCTGGTGGTTTCTATAACAGAGAGTCCTGCTTCAGAAAGCATGTCTA
TCAGCTTGATTTTCACTGGAGTAGATACGATATTCTTTTCATTTTGTAGTCCATCTCGGGGACCAACTTCCACAATTTTC
ACCCGCTTTGGTAAAGTGCCCATGGTTTATTCCTCCTTATTTAATCGATACATTAATATATACCTCTTTAATTTTTAATA
ATAAAGTTA
>CG96859-08-prot 301 aa

(SEQ ID NO:661)

TMGTLPKRVKIVEVGPRDGLQNEKNIVSTPVKIKLIDMLSEAGLSVIETTSFVSPKWVPQMGDHTEVLKGIQKFPGINYP
VLTPNLKGFEAAVAAGAKEVVIFGAASELFTKKNINCSIEESFQRFDAILKAAQSANISVRGYVSCALGCPYEGKISPAK
VAEVTKKFYSMGCYEISLGDTIGVGTPGIMKDMLSAVMQEVPLAALAVHCHDTYGQALANTLMALQMGVSVVDSSVAGLG
GCPYAQGASGNLATEDLVYMLEGLGIHTGVNLQKLLEAGNFICQALNRKTSSKVAQATCKL
>CG96859-09, 987 nt

(SEQ ID NO:662)

TAACTTTATTATTAAAAATTAAAGAGGTATATATTAATGTATCGATTAAATAAGGAGGAATAAACCATGGGCCACCATCA
CCACCATCACACTTTACCAAAGCGGGTGAAAATTGTGGAAGTTGGTCCCCGAGATGGACTACAAAATGAAAAGAATATCG
TATCTACTCCAGTGAAAATCAAGCTGATAGACATGCTTTCTGAAGCAGGACTCTCTGTTATAGAAACCACCAGCTTTGTG
TCTCCTAAGTGGGTTCCCCAGATGGGTGACCACACTGAAGTCTTGAAGGGCATTCAGAAGTTTCCTGGCATCAACTACCC
AGTCCTGACCCCAAATTTGAAAGGCTTCGAGGCAGCGGTTGCTGCTGGAGCCAAGGAAGTAGTCATCTTTGGAGCTGCCT
CAGAGCTCTTCACCAAGAAGAACATCAATTGTTCCATAGAGGAGAGTTTTCAGAGGTTTGACGCAATCCTGAAGGCAGCG
CAGTCAGCCAATATTTCTGTGCGGGGGTACGTCTCCTGTGCTCTTGGCTGCCCTTATGAAGGGAAGATCTCCCCAGCTAA
AGTAGCTGAGGTCACCAAGAAGTTCTACTCAATGGGCTGCTACGAGATCTCCCTGGGGGACACCATTGGTGTGGGCACCC
CAGGGATCATGAAAGACATGCTGTCTGCTGTCATGCAGGAAGTGCCTCTGGCTGCCCTGGCTGTCCACTGCCATGACACC
TATGGTCAAGCCCTGGCCAACACCTTGATGGCCCTGCAGATGGGAGTGAGTGTCGTGGACTCTTCTGTGGCAGGACTTGG
AGGCTGTCCCTACGCACAGGGGGCATCAGGAAACTTGGCCACAGAAGACCTGGTCTACATGCTAGAGGGCTTGGGCATTC
ACACGGGTGTGAATCTCCAGAAGCTTCTGGAAGCTGGAAACTTTATCTGTCAAGCCCTGAACAGAAAAACTAGCTCCAAA
GTGGCTCAGGCTACCTGTAAACTCTGA
>CG96859-09-prot 307 aa

(SEQ ID NO:663)

TMGHHHHHHTLPKRVKIVEVGPRDGLQNEKNIVSTPVKIKLIDMLSEAGLSVIETTSFVSPKWVPQMGDHTEVLKGIQKF
PGINYPVLTPNLKGFEAAVAAGAKEVVIFGAASELFTKKNINCSIEESFQRFDAILKAAQSANISVRGYVSCALGCPYEG
KISPAKVAEVTKKFYSMGCYEISLGDTIGVGTPGIMKDMLSAVMQEVPLAALAVHCHDTYGQALANTLMALQMGVSVVDS
SVAGLGGCPYAQGASGNLATEDLVYMLEGLGIHTGVNLQKLLEAGNFICQALNRKTSSKVAQATCKL

[image]

Rationale for Use as a Diagnostic and/or Target for Small Molecule Drugs and Antibody Therapeutics.

The following is a summary of the findings from the discovery studies, supplementary investigations and assays that also incorporates knowledge in the scientific literature. Taken in total, the data indicates that an inhibitor/antagonist of the human HMG-COA LYASE would be beneficial in the treatment of obesity and/or diabetes.

Mitochondrial 3-Hydroxy-3methylglutaryl coenzyme A lyase (mHMG-CoA lyase) is upregulated in the liver of SHR and WKY rats after triglitazone treatment. mHMG-CoA lyase is the final step in ketogenesis and leucine catabolism which has 3-hydroxy-methylglutaryl-CoA as its substrate, and produces acetoacetate (ketone body) and acetyl-CoA. This process takes place in the liver especially during weight loss and the amount of acetyl-CoA produced during both fatty acid oxidation and ketogenesis often exceeds the capacity of the TCA cycle. Moreover, excess citrate shunts acetyl-CoA back into the cytoplasm where it is used for cholesterol and fatty acid biosynthesis. Therefore, inhibiting this enzyme during weight loss may slow down ketone body formation and the generation of acetyl-CoA, and thus prevent the saturation of the TCA cycle.

The sequence of Acc. No. CG96859-03 was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof.

N. Human Aryl Hydrocarbon Receptor-Like Protein—CG105355-01

The following is a summary of the findings from the discovery studies, supplementary investigations and assays that also incorporates knowledge in the scientific literature. Taken in total, the data indicates that an inhibitor/antagonist of the human Aryl Hydrocarbon Receptor would be beneficial in the treatment of obesity and/or diabetes.

Aryl Hydrocarbon was upregulated 1.9 fold in sub-cutaneous adipose from gestational diabetics. TCDD, an AHR agonist, suppresses PPAR-γ. Conversely TZDs activate PPAR-γ.

AHR activation decreases GLUT4 expression in adipose. The clinical rise may represent a compensatory response. No dysregulation of toxification genes (CYP1A1, CYP1A2, or CYP1B). Upregulated in obese, hyperglycemic mouse liver and adipose. AHR nuclear translocator (ARNT) is also upregulated. AHR interacting protein (AIP) is also upregulated. An AHR antagonist would be beneficial for obesity and diabetes.

Discovery Process: The following sections describe the study design(s) and the techniques used to identify the Aryl Hydrocarbon Receptor-encoded protein and any variants, thereof, as being suitable as diagnostic markers, targets for an antibody therapeutic and targets for a small molecule drugs for Obesity and Diabetes.

| | | |
|---|---|---|
| Studies: | MB.09: | Gestational Diabetes in Humans |
| | BP24.02 | Dietary Induced Obesity in Mice |

Study Statements: MB.09—Gestational diabetes complicates 4% of pregnancies and is a prognostic factor in the development of Type II diabetes. In addition, offspring of women who develop gestational diabetes are at increased risk of becoming obese and developing diabetes. Thus, the differences in gene expression from the metabolic tissues of gestational diabetics and non-diabetic should reveal underlying differences related to the pathophysiology of diabetes. Because many women deliver by C-section this patient population provides an opportunity to examine gene expression changes in surgical material from normals, gestational diabetics treated by diet alone and gestational diabetics treated with insulin. These patients, generally, do not suffer from confounding medical conditions and are not exposed to drugs that may influence gene expression. In this IRB-approved study, clinical information and samples were obtained from sub-cutaneous adipose, skeletal muscle, visceral adipose (omentum) and smooth muscle (uterus) from women giving birth by non-emergency C-section. Maternal and cord blood were also obtained for genotype analysis. The body mass index spanned a wide range in this patient population. Those patients meeting the diagnostic criteria for gestational diabetes were treated with either dietary modification and/or insulin therapy.

BP24.02—The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity.

| | | | |
|---|---|---|---|
| Species #1 | Humans | Strains | N/A |
| Species #2 | Mouse | Strains | C57BL/6J |

Aryl Hydrocarbon Receptor: The Aryl Hydrocarbon Receptor (AHR) is a ligand-dependent transcription factor. 2,3,7,8-tetrachlorodibenzo-p-dioxin (TCDD) is a known activating ligand that initiates expression of multiple genes, including CYP1B1 and glutathione S-transferase. The Aryl Hydrocarbon Receptor forms a heterodimer with ARNT, a nuclear translocator, to form an active complex that crosses the nuclear membrane and binds to DNA. As a result of activation of AHR, PPAR-gamma can become suppressed and GLUT4 expression becomes down regulated in adipose tissue. These actions are of biological importance in the development of insulin resistance and: of diabetes.

The Aryl Hydrocarbon Receptor is a member of the PAS (Per-Ahr-Sim) superfamily of transcription factors having functions in development and detoxification. Only recently has any member of this family been associated with obesity and diabetes.

SPECIES #1 A gene fragment of the human Aryl Hydrocarbon Receptor was initially found to be up-regulated by 1.9 fold in the adipose tissues of human gestational diabetics relative to normal pregnant females using CuraGen's GeneCalling™ method of differential gene expression. A differentially expressed human gene fragment migrating, at approximately 131 nucleotides in length (Table N1. black trace-vertical line) was identified as a component of the human Aryl Hydrocarbon Receptor cDNA. The method of competitive PCR was used for conformation of the gene assessment. The chromatographic peaks corresponding to the gene fragment of the human Aryl Hydrocarbon Receptor are ablated when a gene-specific primer (see below) competes with primers in the linker-adaptors during the PCR amplification. The peaks at 131 nt in length are ablated (gray trace) in the sample from both the gestational diabetics and normal patients.

SPECIES #2 Additionally, gene fragments corresponding to the mouse ortholog of AHR and two AHR-binding proteins, ARNT (AHR nuclear transporter) and AIP (AHR interacting protein) were found to have altered expression in a mouse model of dietary-induced obesity. The altered expression of these genes in the animal model support the role of the Aryl Hydrocarbon Receptor in the pathogenesis of obesity and/or diabetes.

[image]

The chromatograms below represent the competitive PCR result for the Human Aryl Hydrocarbon Receptor (Discovery Study MB.09). The chromatographic peaks corresponding to the gene fragment of the human Aryl Hydrocarbon Receptor (black trace) are ablated when a gene-specific primer (designed from the sequenced band or available databases; below) competes with primers in the linker-adaptors during the PCR amplification. The peaks at 131 nt in length are ablated (gray trace) in the sample from both the gestational diabetics (top chromatogram) and normal patients (bottom chromatogram). This is a confirmatory result. Related Result—Mouse AHR-Interacting protein [AIP] (Discovery Study BP24.02). Related Result—Mouse Aryl hydrocarbon receptor nuclear translocator protein [ARNT] (Discovery Study BP24.02)

The Sequence of CG105355-01 is the reverse complement of the Human Aryl Hydrocarbon Receptor cDNA. The gene fragment (band size: 131 nucleotides in length) identified as being differentially expressed corresponds to nucleotides 187 to 317 (bold) in the 3′ UTR of the cDNA. The gene-specific primer used in the competitive PCR reaction is underlined.

TABLE N2
CG105355-01 Sense and Antisense Sequence

(SEQ ID NO:667)

AGTCGCTGGGGAGTCCCGTCGACGCTCTGTTCCGAGAGCGTGCCCCGGACCGCCAGCTCAGAACAGGGGCAGCCGTGTAG
CCGAACGGAAGCTGGGAGCAGCCCGGACTGGTGGCCCGCGCCCGAGCTCCGCAGGCGGGAAGCACCCTGGATTTCGGAAG
TCCCGGGAGCAGCGCGGCGGCACCTCCCTCACCCAAGGGGCCGCGGCGACCGTCACGGGGCCCGGCGCCACCGTGAGCGA
CCCAGGCCAGGATTCTAAATAGACGGCCCAGGCTCCTCCTCCGCCCGGGCCGCCTCACCTGCGGGCATTGCCGCGCCGCC
TCCGCCGGTGTAGACGGCACCTGCGCCGCCTTGCTCGCGCGTCTCCGCCCCTCGCCCACCCTCACTGCGCCAGGCCCAGG
CAGCTCACCTGTACTGGCGCGGGCTGCGGAAGCCTGCGTGAGCCGAGGCGTTGAGGCGCGGCGCCCACGCCACTGTCCCG
AGAGGACGCAGGTGGAGCGGGCGCGGCTTCGCGGAACCCGGCGCCGGCCGCCGCAGTGGTCCCAGCCTACACCGGGTTCC
GGGGACCCGGCCGCCAGTCCCCGGGGAGTAGCCGCCGCCGTCGGCTGGGCACCATGAACAGCAGCAGCGCCAACATCACC
TACGCCAGTCGCAAGCGGCGGAAGCCGGTGCAGAAAACAGTAAAGCCAATCCCAGCTGAAGGAATCAAGTCAAATCCTTC
CAAGCGGCATAGAGACCGACTTAATACAGAGTTGGACCGTTTGGCTAGCCTGCTGCCTTTCCCACAAGATGTTATTAATA
AGTTGGACAAACTTTCAGTTCTTAGGCTCAGCGTCAGTTACCTGAGAGCCAAGAGCTTCTTTGATGTTGCATTAAAATCC
TCCCCTACTGAAAGAAACGGACGCCAGGATAACTGTAGAGCAGCAAATTTCAGAGAACGCCTGAACTTACAAGAAGGAGA
ATTCTTATTACAGGCTCTGAATGGCTTTGTATTAGTTGTCACTACAGATGCTTTGCTCTTTTATGCTTCTTCTACTATAC
AAGATTATCTAGGGTTTCAGCAGTCTGATGTCATACATCAGAGTGTATATGAACTTATCCATACCGAAGACCGAGCTGAA
TTTCAGCGTCAGCTACACTGGGCATTAAATCCTTCTCAGTGTACAGAGTCTGGACAAGGAATTGAAGAAGCCACTGGTCT
CCCCCAGACAGTAGTCTGTTATAACCCAGACCAGATTCCTCCAGAAAACTCTCCTTTAATGGAGAGGTGCTTCATATGTC
GTCTAAGGTGTCTGCTGGATAATTCATCTGGTTTTCTGGCAATGAATTTCCAAGGGAAGTTAAAGTATCTTCATGGACAG
AAAAAGAAAGGGAAACATCGATCAATACTTCCACCTCAGTTGGCTTTGTTTGCGATAGCTACTCCACTTCAGCCACCATC
CATACTTGAAATCCGGACCAAAAATTTTATCTTTAGAACCAAACACAAACTAGACTTCACACCTATTGGTTGTGATGCCA
AAGGAAGAATTGTTTTAGGATATACTGAAGCAGAGCTGTGCACGAGAGGCTCAGGTTATCAGTTTATTCATCCAGCTGAT
ATGCTTTATTGTGCCGAGTCCCATATCCGAATGATTAAGACTGGAGAAAGTGGCATGATAGTTTTCCGGCTTCTTACAAA
AAACAACCGATGGACTTGGGTGCAGTCTAATGCACGCCTGCTTTATAAAAATGGAAGACCAGATTATATCATTGTAACTC
AGAGACCACTAACAGATGAGGAAGGAACAGAGCATTTACGAAAACGAAATACGAAGTTGCCTTTTATGTTTACCACTCGA
GAAGCTGTGTTGTATGAGGCAACCAACCCTTTTCCTGCCATAATGGATCCCTTACCACTAAGGACTAAAAATGGCACTAG
TGGAAAAGACTCTGCTACCACATCCACTCTAAGCAAGGACTCTCTCAATCCTAGTTCCCTCCTGGCTGCCATGATGCAAC
AAGATGAGTCTATTTATCTCTATCCTGCTTCAAGTACTTCAAGTACTGCACCTTTTGAAAACAACTTTTTCAACGAATCT
ATGAATGAATGCAGAAATTGGCAAGATAATACTGCACCGATGGGAAATGATACTATCCTGAAACATGAGCAAATTGACCA
GCCTCAGGATGTGAACTCATTTGCTGGAGGTCACCCAGGGCTCTTTCAAGATAGTAAAAACAGTGACTTGTACAGCATAA
TGAAAAACCTAGGCATTGATTTTGAAGACATCAGACACATGCAGAATGAAAAATTTTTCAGAAATGATTTTTCTGCTGAG
GTTGACTTCAGAGACATTGACTTAACGGATGAAATCCTGACGTATGTCCAAGATTCTTTAAGTAAGTCTCCCTTCATACC
TTCAGATTATCAACAGCAACAGTCCTTGGCTCTGAACTCAAGCTGTATCGTACAGGAACACCTACATCTAGAACAGCAAC
AGCAACATCACCAAAAGCAAGTAGTAGTGGAGCCACAGCAACAGCTGTGTCAGAAGATGAAGCACATGCAAGTTAATGGC
ATGTTTGAAAATTGGAACTCTAACCAATTCGTGCCTTTCAATTGTCCACAGCAAGACCCACAACAATATAATGTCTTTAC
AGACTTACATGGGATCAGTCAAGAGTTCCCCTACAAATCTGAAATGGATTCTATGCCTTATACACAGAACTTTATTTCCT
GTAATCAGCCTGTATTACCACAACATTCCAAATGTACAGAGCTGGACTACCCTATGGGGAGTTTTGAACCATCCCCATAC
CCCACTACTTCTAGTTTAGAAGATTTTGTCACTTGTTTACAACTTCCTGAAAACCAAAAGCATGGATTAAATCCACAGTC
AGCCATAATAACTCCTCAGACATGTTATGCTGGGGCCGTGTCGATGTATCAGTGCCAGCCAGAACCTCAGCACACCCACG
TGGGTCAGATGCAGTACAATCCAGTACTGCCAGGCCAACAGGCATTTTTAAACAAGTTTCAGAATGGAGTTTTAAATGAA
ACATATCCAGCTGAATTAAATAACATAAATAACACTCAGACTACCACACATCTTCAGCCACTTCATCATCCGTCAGAAGC
CAGACCTTTTCCTGATTTGACATCCAGTGGATTCCTGTAATTCCAAGCCCAATTTTGACCCTGGTTTTTGGATTAAATTA
GTTTGTGAAGGATTATGGAAAAATAAAACTGTCACTGTTGGACGTCAGCAAGTTCACATGGAGGCATTGATGCATGCTAT
TCACAATTATTCCAAACCAAATTTTAATTTTTGCTTTTAGAAAAGGGAGTTTAAAAATGGTATCAAAATTACATATACTA
CAGTCAAGATAGAAAGGGTGCTGCCACGGAGTGGTGACGTACCGTCTACATTTCACATTATTCTGGGCACCACAAAATAT
ACAAAACTTTATCACCGAAACTAAGATTCTTTTAAATTAGAAAATATTCTCTATTTGAATTATTTCTGTCACAGTAAAAA
TAAAATACTTTGAGTTTTGAGCTACTGGATTCTTATTAGTTCCCCAAATACAAAGTTAGAGAACTAAACTAGTTTTTCCT
ATCATGTTAACCTCTGCTTTTATCTCAGATGTTAAAATAAATGGTTTGGTGCTTTTTATAAAAAGATAATCTCAGTGCTT
TCCTCCTTCACTGTTTCATCTAAGTGCCTCACATTTTTTTCTACCTATAACACTCTAGGATGTATATTTTATATAAACTA
TTCTTTTTCTTTTTTAAATTAATATCTTTCT~CACACAAATATTATTTGTGTTTCCTAAATCCAACCATTTTCATTAATT
CAGGCATATTTTAACTCCACTGCTTACCTACTTTCTTCAGGTAAAGGGCAAATAATGATCGAAAAAATAATTATTTATTA
CATAATTTAGTTGTTTCTAGACTATAAATGTTGCTATGTGCCTTATGTTGAAAAAATTTAAAAGTAAAATGTCTTTCCAA
ATTATTTCTTAATTATTATAAAAATATTAAGACAATAGCACTTAAATTCCTCAACAGTGTTTTCAGAAGAAATAAATATA
CCACTCTTTACCTTTATTGATATCTCCATGATGATAGTTGAATGTTGCAATGTGAAAAATCTGCTGTTAACTGCAACCTT
GTGTATTAAATTGCAAGAAGCTTTATTTCTAGCTTTTTAATTAAGCAAAGCACCCATTTCAATGTGTATAAATTGTCTTT
AAAAACTGTTTTAGACCTATAATCCTTGATAATATATTGTGTTGACTTTATAAATTTCGCTTCTTAGAACAGTGGAAACT
ATGTGTTTTTCTCATATTTGAGGAGTGTTAAGATTGCAGATAGCAAGGTTTCGTGCAAAGTATTGTAATGAGTGAATTGA
ATGGTGCATTGTATAGATATAATGAACAAAATTATTTGTAAGATATTTGCAGTTTTTCATTTTAAAAAGTCCATACCTTA
TATATGCACTTAATTTGTTGGGGCTTTACATACTTTATCAATGTGTCTTTCTAAGAAATCAAGTAATGAATCCAACTGCT
TAAAGTTGGTATTAATAAAAAGACAACCACATAGTTCGTTTACCTTCAAACTTTAGGTTTTTTTAATGATATACTGATCT
TCATTACCAATAGGCAAATTAATCACCCTACCAACTTTACTGTCCTAACATGGTTTAAAAGAAAAAATGACACCATCTTT
TATTCTTTTTTTTTTTTTTTTTGAGAGAGAGTCTTACTCTCCCCCCCAAACTCGAGTGCAGTGGCACAATCTTGGCTCAC
TGCAACCTCTACCTCCTGGGTTCAAGTGATTCTCTTGCCTCAGCCTCCCGAGTTGCTGGGATTGCGGGCATGGTGGCGTG
AGCCTGTAGTCCTAGCTACTCGGGAGGCTGACGCACGAGAATACCCTGAACCTGGGAATCGGAGGTTCCAGGGCCAAGAT
CGCCCCACTGACTCCAGCCTGGCAATAGACCGAGACTCCGTCTCCAAAAAAAAAAAAAAATACAATTTTTATTTCTTTTA
CTTTTTTTAGTAAGTTAATGTATATAAAAATGGCTTCGGACAAAATATCTCTGAGTTCTGTGTATTTTCAGTCAAAACTT
TAAACCTGTAGAATCAATTTAAGTGTTGGAAAAAATTTGTCTGAAACATTTCATAATTTGTTTCCAGCATGAGGTATCTA
AGGATTTAGACCAGACGTCTAGATTAATACTCTATTTTTACATTTAAACCTTTTATTATAAGTCTTACATAAACCATTTT
TGTTACTCTCTTCCACATGTTACTGGATAAATTGTTTAGTGGAAAATAGGCTTTTTAATCATGAATATGATGACAATCAG
TTATACAGTTATAAAATTAAAAGTTTGAAAAGCAATATTGTATATTTTTATCTATATAAAATAACTAAAATGTATCTAAG
AATAATAAAATCACGTTAAACCAAATACACGTTTGTCTGTATTGTTAAGTGCCAAACAAAGGATACTTAGTGCACTGCTA
CATTGTGGGATTTATTTCTAGATGATGTGCACATCTAAGGATATGGATGTGTCTAATTTTAGTCTTTTCCTGTACCAGGT
TTTTCTTACAATACCTGAAGACTTACCAGTATTCTAGTGTATTATGAAGCTTTCAACATTACTATGCACAAACTAGTGTT
TTTCGATGTTACTAAATTTTAGGTAAATGCTTTCATGGCTTTTTTCTTCAAAATGTTACTGCTTACATATATCATGCATA
GATTTTTGCTTAAAGTATGATTTATAATATCCTCATTATCAAAGTTGTATACAATAATATATAATAAAATAACAAATATG
AATAATAAAAAAAAAAAAAAAAA.

(SEQ ID NO:688)

TTTTTTTTTTTTTTTTTATTATTCATATTTGTTATTTTATTATATATThTTGTATACAACTTTGATAATGAGGATATTAT
AAATCATACTTTAAGCAAAAATCTATGCATGATATATGTAAGCAGTAACATTTTGAAGAAAAAACCCATGAAAGCATTTA
CCTAAAATTTAGTAACATCGAAAAACA [symbol] TTTGTGCATAGTAATGTTGAAAGCTTCATAATACACTAGAATACTGGTA
AGTCTTCAGGTATTGTAAGAAAAACCTGGTACACGAAAAGACTAAAATTAGACACATCCATATCCTTAGATGTGCACATC
ATCTAGAAATAAATCCCACAATGTAGCAGTGCACTAAGTATCCTTTGTTTGGCACTTAACAATACAGACAAACGTGTATT
TGGTTTAACGTGATTTTATTATTCTTAGATACATTTTAGTTATTTTATATAGATAAAAATATACAATATTGCTTTTCAAA
CTTTTAATTTTATAACTGTATAACTGATTGTCATCATATTCATGATTAAAAAGCCTATTTTCCACTAAACAATTTATCCA
GTAACATGTGGAAGAGAGTAACAAAAATGGTTTATGTAAGACTTATAATAAAAGGTTTAAATGTAAAAATAGAGTATTAA
TCTAGACCTCTGGTCTAAATCCTTAGATACCTCATGCTGGAAACAAATTATGAAATGTTTCAGACAAATTTTTTCCAACA
CTTAAATTGATTCTACAGGTTTAAAGTTTTGACTGAAAATACACAGAACTCAGAGATTTTTTGTCCGAAGCCATTTTTAT
ATACATTAACTTACTAAAAAAAGTAAAAGAAATAAAAATTGTATTTTTTTTTTTTTTGGAGACGGAGTCTCGGTCTATTG
CCAGGCTGGAGTGCAGTGGGGCCATCTTGGCCCTGCAACCTCCGATTCCCAGGTTCAGGCTATTCTCCTGCCTCAGCCTC
CCGAGTAGCTAGGACTACAGGCTCACGCCACCATGCCCGCAATCCCAGCAACTCGGGAGGCTGACGCAAGAGAATCACTT
GAACCCAGGAGGTAGAGGTTGCAGTGAGCCAAGATTGTGCCACTGCACTCCAGTTTGGGCGGCAGAGTAAGACTCTCTCT
CAAAAAAAAAAAAAAAAAGAATAAAAGATGGTGTCATTTTTTCTTTTAAACCATGTTAGGACAGTAAAGTTGGTAGGGTG
ATTAATTTGCCTATTGGTAATGAAGATCAGTATATCATTAAAAAAACCTAAAGTTTGAAGGTAAACGAACTATGTGGTTG
TCTTTTTATTAATACCAACTTTAAGCAGTTGGATTCATTACTTGATTTCTTACAAAGACACATTGATAAAGTATGTAAAG
CCCCAACAAATTAAGTGCATATATAAGGTATGGACTTTTTAAAATGAAAAACTGCAAATATCTTACAAATAATTTTGTTC
ATTATATCTATACAATGCACCATTCAATTCACTCATTACAATACTTTGCACCAAACCTTGCTATCTGCAATCTTAACACT
CCTCAAATATGAGAAAAACACATAGTTTCCACTCTTCTAAGAAGCGAAATTTATAAAGTCAACACAATATATTATCAAGG
ATTATAGGTCTAAAACAGTTTTTAAAGACAATTTATACACATTGAAATGGGTGCTTTGCTTAATTAAAAAGCTAGAAATA
AAGCTTCTTGCAATTTAATACACAAGGTTGCAGTTAACAGCAGATTTTTCACATTGCAACATTCAACTATCATCATGGAG
ATATCAATAAAGGTAAAGAGTGGTATATTTATTTCTTCTGAAAACACTGTTGAGGAATTTAAGTGCTATTGTCTTAATAT
TTTTATAATAATTAAGAAATAATTTGGAAAGACATTTTACTTTTAAATTTTTTCAACATAAGGCACATAGCAACATTTAT
AGTCTAGAAACAACTAAATTATGTAATAAATAATTATTTTTTCGATCATTATTTGCCCTTTACCTGAAGAAAGTAGGTAA
GCAGTGGAGTTAAAATATGCCTGAATTAATGAAAATGGTTGGATTTAGGAAACACAAATAATATTTGTGTGCAGAAAGAT
ATTAATTTAAAAAAGAAAAAGAATACTTTATATAAAATATACATCCTAGAGTGTTATAGGTAGAAAAAAATGTGAGGCAC
TTAGATGAAACAGTGAAGGAGGAAAGCACTGAGATTATCTTTTTATAAAAAGCACCAAACCATTTATTTTAACATCTGAG
ATAAAAGCAGAGGTTAACATGATAGGAAAAACTAGTTTAGTTCTCTAACTTTGTATTTCGGGAACTAATAAGAATCCAGT
AGCTCAAAACTCAAAGTATTTTATTTTTACTGTGACAGAAATAATTCAAATAGAGAATATTTTCTAATTTAAAAGAATCT
TAGTTTCCCTGATAAAGTTTTGTATATTTTGTGGTGCCCAGAATAATGTGAAATGTAGACGGTACCTCACCACTCCGTGG
CAGCACCCTTTCTATCTTGACTGTAGTATATGTAATTTTGATACCATTTTTAAACTCCCTTTTCTAAAAGCAAAAATTAA
AATTTCGTTTGGAATAATTGTGAATAGCATGCATCAATGCCTCCATGTGAACTTGCTGACGTCCAACAGTGACAGTTTTA
TTTTTCCATAATCCTTCACAAACTAATTTAATCCAAAAACCAGGGTCAAAATTGGGCTTGGAATTACAGGAATCCACTGG
ATCTCAAATCAGGAAAAGGTCTGGCTTCTGACGGATGATGAAGTGGCTGAAGATGTGTCGTACTCTGAGTGTTATTTATG
TTATTTAATTCAGCTGGATATGTTTCATTTAAAACTCCATTCTGAAACTTGTTTAAAAATGCCTGTTGGCCTGGCAGTAC
TGGATTGTACTGCATCTGACCCACGTGGGTGTGCTGAGGTTCTGGCTGGCACTGATACATCGACACGGCCCCAGCATAAC
ATGTCTCAGGAGTTATTATGGCTGACTGTGGATTTAATCCATGCTTTTGGTTTTCAGGAAGTTGTAAACAAGTGACAAAA
TCTTCTAAACTAGAAGTAGTGCGGTATGGGGATGGTTCAAAACTCCCCATAGGGTAGTCCAGCTCTGTACATTTGGAATG
TTGTGGTAATACAGGCTGATTACAGGAAATAAAGTTCTGTGTATAAGGCATAGAATCCATTTCAGATTTGTAGGGGAACT
CTTGACTGATCCCATGTAAGTCTGTAAAGACATTATATTGTTGTGGGTCTTGCTGTGGACAATTGAAAGGCACGAATTGG
TTAGAGTTCCAATTTTCAAACATGCCATTAACTTGCATGTGCTTCATCTTCTGACACAGCTGTTGCTGTGGCTCCACTAC
TACTTGCTTTTGGTGATGTTGCTGTTGCTGTTCTAGATGTAGGTGTTCCTGTACCATACAGCTTGAGTTCAGAGCCAAGG
ACTGTTGCTGTTGATAATCTGAAGGTATGAAGGGAGACTTACTTAAAGAATCTTGGACATACGTCAGGATTTCATCCGTT
AAGTCAATGTCTCTGAAGTCAACCTCACCAGAAAAATCATTTCTGAAAAATTTTTCATTCTGCATGTGTCTGATGTCTTC
AAAATCAATGCCTAGGTTTTTCATTATGCTGTACAAGTCACTGTTTTTACTATCTTGAAACAGCCCTGGGTCACCTCCAG
CAAATGAGTTCACATCCTGAGGCTCGTCAATTTGCTCATGTTTCAGGATAGTATCATTTCCCATCGGTGCAGTATTATCT
TGCCAATTTCTGCATTCATTCATAGATTCGTTGAAAAAGTTGTTTTCAAAACGTGCAGTACTTGAAGTACTTGAAGCAGG
ATAGAGATAAATAGACTCATCTTGTTGCATCATGGCAGCCAGGAGGGAACTAGGATTGAGAGAGTCCTTGCTTAGAGTGG
ATGTGGTAGCAGAGTCTTTTCCACTAGTGCCATTTTTAGTCCTTAGTGGTAAGGGATCCATTATGGCAGCAAAAGGGTTG
GTTGCCTCATACAACACAGCTTCTCCAGTGGTAAACATAAAAGGCAACTTCGTATTTCGTTTTCGTAAATGCTCTGTTCC
TTCCTCATCTGTTAGTGGTCTCTGAGTTACAATGATATAATCTGGTCTTCCATTTTTATAAAGCAGGCGTGCATTAGACT
GGACCCAAGTCCATCGGTTGTTTTTTGTAAGAAGCCGGAAAACTATCATGCCACTTTCTCCAGTCTTAATCATTCGGATA
TGGGACTCGGCACAATAAAGCATATCAGCTGCATGAATAAACTGATAACCTGAGCCTCTCGTGCACAGCTCTGCTTCAGT
ATATCCTAAAACAATTCTTCCTTTCGCATCACAACCAATAGGTGTGAAGTCTAGTTTGTGTTTGGTTCTAAAGATAAAAT
TTTTGGTCCGGATTTCAAGTATGGATGGTGGCTGAAGTGGAGTAGCTATCGCAAACAAAGCCAACTGAGGTGGAAGTATT
GATCCATCTTTCCCTTTCTTTTTCTGTCCATGAAGATACTTTAACTTCCCTTGGAAATTCATTGCCAGAAAACCAGATGA
ATTATCCAGCAGACACCTTAGACGACATATGAAGCACCTCTCCATTAAAGGAGAGTTTTCTGGAGGAATCTGGTCTGGGT
TATAACAGACTACTGTCTGGCGGAGACCAGTGGCTTCTTCAATTCCTTGTCCAGACTCTGTACACTGAGAACGATTTAAT
GCCCAGTGTAGCTGACGCTGAAATTCAGCTCGGTCTTCGGTATGGATAAGTTCATATACACTCTGATGTATGACATCAGA
CTGCTGAAACCCTAGATAATCTTGTATAGTAGAAGAAGCATAAAAGACCAAAGCATCTGTAGTGACAACTAATACAAAGC
CATTCAGAGCCTGTAATAACAATTCTCCTTCTTGTAAGTTCAGGCCTTCTCTGAAATTTGCTGCTCTACAGTTATCCTGG
CCTCCGTTTCTTTCAGTAGGGGAGGATTTTAATGCAACATCAAAGAAGCTCTTGGCTCTCAGGTAACTGACGCTGAGCCT
AAGAACTGAAAGTTTGTCCAACTTATTAATAACATCTTGTGGGAAAGGCAGCAGGCTAGCCAAACGGTCCAACTCTGTAT
TAAGTCGGTCTCTATGCCGCTTGGAAGGATTTGACTTGATTCCTTCAGCTGGGATTGGCTTTACTGTTTTCTGCACCGGC
TTCCGCCGCTTGCGACTGGCGTACGTGATGTTCGCGCTGCTGCTGTTCATCGTGCCCAGCCGACGGCGGCGGCTACTCCC
CGGGCACTGGCGGCCGGGTCCCCGGAACCCGGTGTAGGCTGGGACCACTGCGGCGGCCGGCGCCCGGTTCCGCGAAGCCG
CGCCCGCTCCACCTGCGTCCTCTCGGGACAGTGGCGTGGGCGCCGCGCCTCAACGCCTCCGCTCACGCAGGCTTCCGCAG
CCCGCGCCAGTACACGTGAGCTGCCTGGGCCTGGCCCAGTGACGGTGGGCGAGGGGCGGAGACCCGCGAGCAAGGCGGCG
CAGGTGCCGTCTACACCGGCGGAGGCGGCGCGGCAATGCCCGCAGGTGAGGCGGCCCGGGCGGAGGAGGAGCCTGGGCCG
TCTATTTTGAATCCTGGCCTGGGTCGCTCACGGTGGCGCCGCGCCCCCTGACCGTCGCCGCGGCCCCTTGGGTGAGGGAG
GTGCCGCCGCGCTGCTCCCGGGACTTCCCAAATCCACCGTGCTTCCCGCCTGCGGAGCTCGGGCGCGGGCCACCAGTCCC
GGCTGCTCCCAGCTTCCGTTCGGCTACACGGCTGCCCCTGTTCTGAGCTCGCGGTCCGGGGCACGCTCTCGGAACAGAGC
GTCGACGGGACTCCCCAGCCACT

TABLE N3
Human Aryl Hydrocarbon Receptor Protein Sequence:

ORF Start: 615```ORF Stop: 3159```Frame: -3
>CG105355-01-prot 848 aa

(SEQ ID NO:669)

MNSSSANITYASRKRRKPVQKTVKPIPAEGIKSNPSKRHRDRLNTELDRLASLLPFPQDVINKLDKLSVLRLSVSYLRAK
SFFDVALKSSPTERNGGQDNCRAANFREGLNLQEGEFLLQALNGFVLVVTTDALVFYASSTIQDYLGFQOSDVIHQSVYE
LIHTEDRAEFQRQLHWALNPSQCTESGQGIEEATGLPQTVVCYNPDQIPPENSPLMERCFICRLRCLLDNSSGFLAMNFQ
GKLKYLHGQKKKGKDGSILPPQLALFAIATPLQPPSILEIRTKNFIFRTKHKLDFTPIGCDAKGRIVLGTYEAELCTRGS
GYQFIHAADMLYCAESHIRMIKTGESGMIVFRLLTKNNRWTWVQSNARLLYKNGRPDYIIVTQRPLTDEEGTEHLRKRNT
KLPFMFTTGEAVLYEATNPFPAIMDPLPLRTKNGTSGKDSATTSTLSKDSLNPSSLLAAMMQQDESIYLYPASSTSSTAP
FENNFFNESMNECRNWQDWTAPMGNDTILKHEQIDQPQDVNSFAGGHPGLFQDSKNSDLYSIMKNLGIDFEDIRHMQNEK
FFRNDFSGEVDFRDIDLTDEILTYVQDSLSKSPFIPSDYQQQQSLALNSSCMVQEHLHLEQQQQHHQKQVVVEPQQQLCQ
KMKHMQVNGMFENWNSNQFVPFNCPQQDPQQYNVFTDLHGISQEFPYKSEMDSMPYTQNFISCNQPVLPQHSKCTELDYP
MGSFEPSPYPTTSSLEDFVTCLQLPENQKHGLNPQSAIITPQTCYAGAVSMYQCQPEPQHTHVGQMQYNPVLPGQQAFLN
KFQNGVLNETYPAELNNINNTQTTTHLQPLHHPSEARPFPDLTSSGFL

The following is an alignment of the protein sequences of the human, rat and mouse versions of the Aryl Hydrocarbon Receptor.

[image]

In addition to the human version of the Aryl Hydrocarbon Receptor identified as being differentially expressed in the experimental study, other variants have been identified by direct sequencing of cDNAs derived from many different human tissues and from sequences in public databases. No splice-form variants have been identified at CuraGen whereas several amino acid-changing cSNPs were identified. These are found below. The preferred variant of all those identified, to be used for screening purposes, is CG105355-01.

TABLE N5

The variants of the human Aryl Hydrocarbon
Receptor obtained from direct cloning and/or
public databases.

| | | | | | |
|---|---|---|---|---|---|
| DNA | | | | | |
| Posi- | | | AA | AA | public |
| tion | Strand | Alleles | Position | Change | SNP # |

| | | | | | |
|---|---|---|---|---|---|
| 757 | Plus | A:G | 48 | Asp => Gly | — |
| 869 | Plus | T:C | 85 | Val => Val | — |
| 1132 | Plus | A:G | 173 | Gln => Arg | — |
| 2028 | Plus | G:A | 472 | Ala => Thr | — |
| 2275 | plus | G:A | 554 | Arg => Lys | rs2066853 |

RTQ-PCR Relative Expression Levels of Human Aryl Hydrocarbon Receptor (CG105355-01).

Tissue expression for the human Aryl Hydrocarbon Receptor was assessed using the primer-probe set Ag4285, described in Table NAA. Results of the RTQ-PCR runs are shown in Tables NAB, NAC and NAD.

TABLE NAA

Probe Name Ag4285

| | | | | |
|---|---|---|---|---|
| | | | Start | SEQ ID |
| Primers | Sequences | Length | Position | NO |

| | | | | |
|---|---|---|---|---|
| Forward | 5′-caggatttcatccgttaagtca-3′ | 22 | 1765 | 673 |
| Probe | TET-5′-tgtctctgaagtcaacctcaccagaa-3′-TAMRA | 26 | 1738 | 674 |
| Reverse | 5′-acatcagacacatgcagaatga-3′ | 22 | 1695 | 675 |

The highest level of expression in normal, adult tissue is in adipose.

TABLE NAB
General_screening_panel_v1.4

| | | Rel. Exp. (%) | Rel. Exp. |
|---|---|---|---|
| | Tissue Name | Ag4285 | (%) Ag4285 |
| 1. | Adipose | 26.19 | 11.7% |
| 2. | Melanoma* Hs688(A).T | 27.69 | 4.2% |
| 3. | Melanoma* Hs688(B).T | 26.66 | 8.5% |
| 4. | Melanoma* M14 | 25.74 | 16% |
| 5. | Melanoma* LOXIMVI | 28.26 | 2.8% |
| 6. | Melanoma* SK-MEL-5 | 25.93 | 14.1% |
| 7. | Squamous cell carcinoma SCC-4 | 25.99 | 13.5% |
| 8. | Testis Pool | 28.95 | 1.7% |
| 9. | Prostate ca.* (bone met) PC-3 | 25.65 | 17.1% |
| 10. | Prostate Pool | 28.34 | 2.6% |
| 11. | Placenta | 27.54 | 4.6% |
| 12. | Uterus Pool | 27.81 | 3.8% |
| 13. | Ovarian ca. OVCAR-3 | 28.54 | 2.3% |
| 14. | Ovarian ca. SK-OV-3 | 27.67 | 4.2% |
| 15. | Ovarian ca. OVCAR-4 | 29.17 | 1.5% |
| 16. | Ovarian ca. OVCAR-5 | 25 | 26.8% |
| 17. | Ovarian ca. IGROV-1 | 28.39 | 2.6% |
| 18. | Ovarian ca. OVCAR-8 | 30.62 | .5% |
| 19. | Ovary | 27.79 | 3.9% |
| 20. | Breast ca. MCF-7 | 26.83 | 7.5% |
| 21. | Breast ca. MDA-MB-231 | 25.65 | 17.1% |
| 22. | Breast ca. BT 549 | 23.94 | 55.9% |
| 23. | Breast ca. T47D | 24.51 | 37.6% |
| 24. | Breast ca. MDA-N | 26.81 | 7.6% |
| 25. | Breast Pool | 27.3 | 5.4% |
| 26. | Trachea | 26.57 | 9% |
| 27. | Lung | 29.07 | 1.6% |
| 28. | Fetal Lung | 24.25 | 45.1% |
| 29. | Lung ca. NCI-N417 | 36.08 | 0% |
| 30. | Lung ca. LX-1 | 26.87 | 7.3% |
| 31. | Lung ca. NCI-H146 | 30.12 | .8% |
| 32. | Lung ca. SHP-77 | 27.72 | 4.1% |
| 33. | Lung ca. A549 | 26.37 | 10.4% |
| 34. | Lung ca. NCI-H526 | 34.54 | 0% |
| 35. | Lung ca. NCI-H23 | 25.02 | 26.4% |
| 36. | Lung ca. NCI-H460 | 26.81 | 7.6% |
| 37. | Lung ca. HOP-62 | 26.36 | 10.4% |
| 38. | Lung ca. NCI-H522 | 33.54 | .1% |
| 39. | Liver | 31.8 | .2% |
| 40. | Fetal Liver | 27.41 | 5% |
| 41. | Liver ca. HepG2 | 26.76 | 7.9% |
| 42. | Kidney Pool | 27.13 | 6.1% |
| 43. | Fetal Kidney | 26.34 | 10.6% |
| 44. | Renal ca. 786-0 | 26.66 | 8.5% |
| 45. | Renal ca. A498 | 27.77 | 3.9% |
| 46. | Renal ca. ACHN | 28.33 | 2.7% |
| 47. | Renal ca. UO-31 | 26.22 | 11.5% |
| 48. | Renal ca. TK-10 | 26.13 | 12.2% |
| 49. | Bladder | 26.18 | 11.8% |
| 50. | Gastric ca. (liver met.) NCI-N87 | 24.48 | 38.4% |
| 51. | Gastric ca. KATO III | 23.29 | 87.7% |
| 52. | Colon ca. SW-948 | 27.39 | 5.1% |
| 53. | Colon ca. SW480 | 27.16 | 6% |
| 54. | Colon ca.* (SW480 met) SW620 | 27.59 | 4.5% |
| 55. | Colon ca. HT29 | 27.31 | 5.4% |
| 56. | Colon ca. HCT-116 | 27.07 | 6.4% |
| 57. | Colon ca. CaCo-2 | 26.09 | 12.6% |
| 58. | Colon cancer tissue | 25.67 | 16.8% |
| 59. | Colon ca. SW1116 | 30.22 | .7% |
| 60. | Colon ca. Colo-205 | 30.17 | .7% |
| 61. | Colon ca. SW-48 | 28.35 | 2.6% |
| 62. | Colon Pool | 27.11 | 6.2% |
| 63. | Small Intestine Pool | 28.03 | 3.3% |
| 64. | Stomach Pool | 27.79 | 3.9% |
| 65. | Bone Marrow Pool | 28 | 3.3% |
| 66. | Fetal Heart | 28.14 | 3% |
| 67. | Heart Pool | 28.12 | 3.1% |
| 68. | Lymph Node Pool | 27.48 | 4.8% |
| 69. | Fetal Skeletal Muscle | 28.25 | 2.8% |
| 70. | Skeletal Muscle Pool | 30.01 | .8% |
| 71. | Spleen Pool | 27.5 | 4.7% |
| 72. | Thymus Pool | 28 | 3.3% |
| 73. | CNS cancer (glio/astro) U87-MG | 25.11 | 24.8% |
| 74. | CNS cancer (glio/astro) U-118-MG | 24.42 | 40.1% |
| 75. | CNS cancer (neuro; met) SK-N-AS | 27.51 | 4.7% |
| 76. | CNS cancer (astro) SF-539 | 28.74 | 2% |
| 77. | CNS cancer (astro) SNB-75 | 26 | 13.4% |
| 78. | CNS cancer (glio) SNB-19 | 28.4 | 2.5% |
| 79. | CNS cancer (glio) SF-295 | 23.1 | 100% |
| 80. | Brain (Amygdala) Pool | 29.62 | 1.1% |
| 81. | Brain (cerebellum) | 30.31 | .7% |
| 82. | Brain (fetal) | 30.3 | .7% |
| 83. | Brain (Hippocampus) Pool | 29.35 | 1.3% |
| 84. | Cerebral Cortex Pool | 29.3 | 1.4% |
| 85. | Brain (Substantia nigra) Pool | 30.13 | .8% |
| 86. | Brain (Thalamus) Pool | 28.91 | 1.8% |
| 87. | Brain (whole) | 30.18 | .7% |
| 88. | Spinal Cord Pool | 29.3 | 1.4% |
| 89. | Adrenal Gland | 28.43 | 2.5% |
| 90. | Pituitary gland Pool | 31.08 | .4% |
| 91. | Salivary Gland | 30.98 | .4% |
| 92. | Thyroid (female) | 28.28 | 2.8% |
| 93. | Pancreatic ca. CAPAN2 | 26.82 | 7.6% |
| 94. | Pancreas Pool | 27.11 | 6.2% |

The highest level of expression in normal (non-pregnant) adult tissue is adipose.

TABLE NAC
Panel 5 Islet

| | | Rel. Exp. | Rel. Exp. |
|---|---|---|---|
| | Tissue Name | (%) Ag4285 | (%) Ag4285 |
| 1. | 97457_Patient-02go_adipose | 33.78 | 1.8% |
| 2. | 97476_Patient-07sk_skeletal muscle | 30.6 | 15.9% |
| 3. | 97477_Patient-07ut_uterus | 32.87 | 3.3% |
| 4. | 97478_Patient-07pl_placenta | 28.14 | 87.7% |
| 5. | 99167_Bayer Patient 1 | 33.04 | 2.9% |
| 6. | 97482_Patient-08ut_uterus | 32.16 | 5.4% |
| 7. | 97483_Patient-08pl_placenta | 28.42 | 72.2% |
| 8. | 97486_Patient-09sk_skeletal muscle | 33.31 | 2.4% |
| 9. | 97487_Patient-09ut_uterus | 30.83 | 13.6% |
| 10. | 97488_Patient-09pl_placenta | 29.07 | 46% |
| 11. | 97492_Patient-10ut_uterus | 31.14 | 11% |
| 12. | 97493_Patient-10pl_placenta | 27.95 | 100% |
| 13. | 97495_Patient-11go_adipose | 30.15 | 21.8% |
| 14. | 97496_Patient-11sk_skeletal muscle | 32.37 | 4.7% |
| 15. | 97497_Patient-11ut_uterus | 30.81 | 13.8% |
| 16. | 97498_Patient-11pl_placenta | 30.78 | 14.1% |
| 17. | 97500_Patient-12go_adipose | 30.16 | 21.6% |
| 18. | 97501_Patient-12sk_skeletal muscle | 31.73 | 7.3% |
| 19. | 97502_Patient-12ut_uterus | 31.19 | 10.6% |
| 20. | 97503_Patient-12pl_placenta | 29.22 | 41.5% |
| 21. | 94721_Donor 2 U - A_Mesenchymal | 30.94 | 12.6% |
| | Stem Cells | | |
| 22. | 94722_Donor 2 U - B_Mesenchymal | 32.08 | 5.7% |
| | Stem Cells | | |
| 23. | 94723_Donor 2 U - C_Mesenchymal | 30.97 | 12.3% |
| | Stem Cells | | |
| 24. | 94709_Donor 2 AM - A_adipose | 30.72 | 14.7% |
| 25. | 94710_Donor 2 AM - B_adipose | 31.17 | 10.7% |
| 26. | 94711_Donor 2 AM - C_adipose | 31.89 | 6.5% |
| 27. | 94712_Donor 2 AD - A_adipose | 29.69 | 29.9% |
| 28. | 94713_Donor 2 AD - B_adipose | 29.72 | 29.3% |
| 29. | 94714_Donor 2 AD - C_adipose | 29.34 | 38.2% |
| 30. | 94742_Donor 3 U - A_Mesenchymal | 31.74 | 7.2% |
| | Stem Cells | | |
| 31. | 94743_Donor 3 U - B_Mesenchymal | 30.93 | 12.7% |
| | Stem Cells | | |
| 32. | 94730_Donor 3 AM - A_adipose | 29.89 | 26.1% |
| 33. | 94731_Donor 3 AM - B_adipose | 30.86 | 13.3% |
| 34. | 94732_Donor 3 AM - C_adipose | 30.81 | 13.8% |
| 35. | 94733_Donor 3 AD - A_adipose | 28.94 | 50.3% |
| 36. | 94734_Donor 3 AD - B_adipose | 31.01 | 12% |
| 37. | 94735_Donor 3 AD - C_adipose | 29.28 | 39.8% |
| 38. | 77138_Liver_HepG2untreated | 28.55 | 66% |
| 39. | 73556_Heart_Cardiac stromal cells | 40 | 0% |
| | (primary) | | |
| 40. | 81735_Small Intestine | 30.48 | 17.3% |
| 41. | 72409_Kidney_Proximal Convoluted | 30.31 | 19.5% |
| | Tubule | | |
| 42. | 82685_Small intestine_Duodenum | 34.39 | 1.2% |
| 43. | 90650_Adrenal_Adrenocortical | 32.43 | 4.5% |
| | adenoma | | |
| 44. | 72410_Kidney_HRCE | 29.75 | 28.7% |
| 45. | 72411_Kidney_HRE | 31.27 | 10% |
| 46. | 73139_Uterus_Uterine smooth muscle | 32.2 | 5.3% |
| | cells | | |

The protein associated with Ahr_CG105355-01 is encoded in a negative reading frame. The sequence shown below has been reverse-complemented and renumbered to allow reading of the protein in the expected N to C direction.

TABLE N7
cDNA Sequence of Translated Protein

Frame: −3—Nucleotide 615 to 3158) with RTQ-PCR Primer/Probe Positions Indicated.

| | |
|---|---|
| CAGTGGCTGGGGAGTCCCGTCGACGCTCTGTTCCGAGAGCGTGCCCCGGACCGCCAGCTCAGAACAGGGGCAGCCGTGTA | (SEQ ID NO:677) |
| GCCGAACGGAAGCTGGGAGCAGCCGGGACTGGTGGCCCGCGCCCGAGCTCCGCAGGCGGGAAGCACCCTGGATTTGGGAA | |
| GTCCCGGGAGCAGCGCGGCGGCACCTCCCTCACCCAAGGGGCCGCGGCGACGGTCACGGGGCGCGGCGCCACCGTGAGCG | |
| ACCCAGGCCAGGATTCTAAATAGACGGCCCAGGCTCCTCCTCCGCCCGGGCCGCCTCACCTGCGGGCATTGCCGCGCCGC | |
| CTCCGCCGGTGTAGACGGCACCTGCGCCGCCTTGCTCGCGGGTCTCCGCCCCTCGCCCACCCTCACTGCGCCAGGCCCAG | |
| GCAGCTCACCTGTACTGGCGCGGGCTGCGGAAGCCTGCGTGAGCCGAGGCGTTGAGGCGCGGCGCCCACGCCACTGTCCC | |
| GAGAGGACGCAGGTGGAGCGGGCGCGGCTTCGCGGAACCCGGCGCCGGCCGCCGCAGTGGTCCCAGCCTACACCGGGTTC | |
| CGGGGACCCGGCCGCCAGTGCCCGGGGAGTAGCCGCCGCCGTCGGCTGGGCACCATGAACAGCAGCAGCGCCAACATCAC | |
| M N S S S A N I T | (SEQ ID NO:676) |
| CTACGCCAGTCGCAAGCGGCGGAAGCCGGTGCAGAAAACAGTAAAGCCAATCCCAGCTGAAGGAATCAAGTCAAATCCTT | |
| Y A S R K R R K P V Q K T V K P I P A E G I K S N P S | |
| CCAAGCGGCATAGAGACCGACTTAATACAGAGTTGGACCGTTTGGCTAGCCTGCTGCCTTTCCCACAAGATGTTATTAAT | |
| K R H R D R L N T E L D R L A S L L P F P Q D V I N | |
| AAGTTGGACAAACTTTCAGTTCTTAGGCTCAGCGTCAGTTACCTGAGAGCCAAGAGCTTCTTTGATGTTGCATTAAAATC | |
| K L D K L S V L R L S V S Y L R A K S F F D V A L K S | |
| CTCCCCTACTGAAAGAAACGGAGGCCAGGATAACTGTAGAGCAGCAAATTTCAGAGAAGGCCTGAACTTACAAGAAGGAG | |
| S P T E R N G G Q D N C R A A N F R E G L N L Q E G E | |
| AATTCTTATTACAGGCTCTCAATGGCTTTGTATTAGTTGTCACTACAGATGCTTTGGTCTTTTATGCTTCTTCTACTATA | |
| F L L Q A L N G F V L V V T T D A L V F Y A S S T I | |
| CAAGATTATCTAGGGTTTCAGCAGTCTGATGTCATACATCAGAGTGTATATGAACTTATCCATACCGAAGACCGAGCTGA | |
| Q D Y L G F Q Q S D V I H Q S V Y E L I H T E D R A E | |
| ATTTCAGCGTCAGCTACACTGGGCATTAAATCCTTCTCAGTGTACAGAGTCTGGACAAGGAATTGAAGAAGCCACTGGTC | |
| F Q R Q L H W A L N P S Q C T E S G Q G I E E A T G L | |
| TCCCCCAGACAGTAGTCTGTTATAACCCAGACCAGATTCCTCCAGAAAACTCTCCTTTAATGGAGAGGTGCTTCATATGT | |
| P Q T V V C Y N P D Q I P P E N S P L M E R C F I C | |
| CGTCTAAGGTGTCTGCTGGATAATTCATCTGGTTTTCTGGCAATGAATTTCCAAGGGAAGTTAAAGTATCTTCATGGACA | |
| R L R C L L D N S S G F L A M N F Q G K L K Y L H G Q | |
| GAAAAAGAAAGGGAAAGATGGATCAATACTTCCACCTCAGTTGGCTTTGTTTGCGATAGCTACTCCACTTCAGCCACCAT | |
| K K K G K D G S I L P P Q L A L F A I A T P L Q P P S | |
| CCATACTTGAAATCCGGACCAAAAATTTTATCTTTAGAACCAAACACAAACTAGACTTCACACCTATTGGTTGTGATGCC | |
| I L E I R T K N F I F R T K H K L D F T P I G C D A | |
| AAAGGAAGAATTGTTTTAGGATATACTGAAGCAGAGCTGTGCACGAGAGGCTCAGGTTATCAGTTTATTCATGCAGCTGA | |
| K G R I V L G Y T E A E L C T R G S G Y Q F I H A A D | |
| TATGCTTTATTGTGCCGAGTCCCATATCCGAATGATTAAGACTGGAGAAAGTGGCATGATAGTTTTCCGGCTTCTTACAA | |
| M L Y C A E S H I R M I K T G E S G M I V F R L L T K | |
| AAAACAACCGATGGACTTGGGTCCAGTCTAATGCACGCCTGCTTTATAAAAATGGAAGACCAGATTATATCATTGTAACT | |
| N N R W T W V Q S N A R L L Y K N G R P D Y I I V T | |
| CAGAGACCACTAACAGATGAGGAAGGAACAGAGCATTTACGAAAACGAAATACGAAGTTGCCTTTTATGTTTACCACTGG | |
| Q R P L T D E E G T E H L R K R N T K L P F M F T T G | |
| AGAAGCTGTGTTGTATGAGGCAACCAACCCTTTTCCTGCCATAATGGATCCCTTACCACTAAGGACTAAAAATGGCACTA | |
| E A V L Y E A T N P F P A I M D P L P L R T K N G T S | |
| GTGGAAAAGACTCTGCTACCACATCCACTCTAAGCAAGGACTCTCTCAATCCTAGTTCCCTCCTGGCTGCCATGATGCAA | |
| G K D S A T T S T L S K D S L N P S S L L A A M M Q | |
| CAAGATGAGTCTATTTATCTCTATCCTGCTTCAAGTACTTCAAGTACTGCACCTTTTGAAAACAACTTTTTCAACGAATC | |
| Q D E S I Y L Y P A S S T S S T A P F E N N F F N E S | |
| TATGAATGAATGCAGAAATTGGCAAGATAATACTGCACCGATGGGAAATGATACTATCCTGAAACATGAGCAAATTGACC | |
| M N E C R N W Q D N T A P M G N D T I L K H E Q I D Q | |
| AGCCTCAGGATGTGAACTCATTTGCTGGAGGTCACCCAGGGCTCTTTCAAGATAGTAAAAACAGTGACTTGTACAGCATA | |
| P Q D V N S F A G G H P G L F Q D S K N S D L Y S I | |
| ATGAAAAACCTAGGCATTGATTTTGAAGACATCAGACACATGCAGAATGAAAAATTTTTCAGAAATGATTTTTCTGGTGA | |
| M K N L G I D F E D I R H M Q N E K F F R N D F S G E | |
| GGTTGACTTCAGAGACATTGACTTAACGGATGAAATCCTGACGTATGTCCAAGATTCTTTAAGTAAGTCTCCCTTCATAC | |
| V D F R D I D L T D E I L T Y V Q D S L S K S P F I P | |
| CTTCAGATTATCAACAGCAACAGTCCTTGGCTCTGAACTCAAGCTGTATGGTACAGGAACACCTACATCTAGAACAGCAA | |
| S D Y Q Q Q Q S L A L N S S C M V Q E H L H L E Q Q | |
| CAGCAACATCACCAAAAGCAAGTAGTAGTGGAGCCACAGCAACAGCTGTGTCAGAAGATGAAGCACATGCAAGTTAATGG | |
| Q Q H H Q K Q V V V E P Q Q Q L C Q K M K H M Q V N G | |
| CATGTTTGAAAATTGGAACTCTAACCAATTCGTGCCTTTCAATTGTCCACAGCAAGACCCACAACAATATAATGTCTTTA | |
| M F E N W N S N Q F V P F N C P Q Q D P Q Q Y N V F T | |
| CAGACTTACATGGGATCAGTCAAGAGTTCCCCTACAAATCTGAAATGGATTCTATGCCTTATACACAGAACTTTATTTCC | |
| D L H G I S Q E F P Y K S E M D S M P Y T Q N F I S | |
| TGTAATCAGCCTGTATTACCACAACATTCCAAATGTACAGAGCTGGACTACCCTATGGGGAGTTTTGAACCATCCCCATA | |
| C N Q P V L P Q H S K C T E L D Y P M G S F E P S P Y | |
| CCCCACTACTTCTAGTTTAGAAGATTTTGTCACTTGTTTACAACTTCCTGAAAACCAAAAGCATGGATTAAATCCACAGT | |
| P T T S S L E D F V T C L Q L P E N Q K H G L N P Q S | |
| CAGCCATAATAACTCCTCAGACATGTTATGCTGGGGCCGTGTCGATGTATCAGTGCCAGCCAGAACCTCAGCACACCCAC | |
| A I I T P Q T C Y A G A V S M Y Q C Q P E P Q H T H | |
| GTGGGTCAGATGCAGTACAATCCAGTACTGCCAGGCCAACAGGCATTTTTAAACAAGTTTCAGAATGGAGTTTTAAATGA | |
| V G Q M Q Y N P V L P G Q Q A F L N K F Q N G V L N E | |
| AACATATCCAGCTGAATTAAATAACATAAATAACACTCAGACTACCACACATCTTCAGCCACTTCATCATCCGTCAGAAG | |
| T Y P A E L N N I N N T Q T T T H L Q P L H H P S E A | |
| CCAGACCTTTTCCTGATTTGACATCCAGTGGATTCCTGTAATTCCAAGCCCAATTTTGACCCTGGTTTTTGGATTAAATT | |
| R P F P D L T S S G F L | |
| ACTTTGTGAAGGATTATGGAAAAATAAAACTGTCACTGTTGGACGTCAGCAAGTTCACATGGAGGCATTGATGCATGCTA | |
| TTCACAATTATTCCAAACCAAATTTTAATTTTTGCTTTTAGAAAAGGGAGTTTAAAAATGGTATCAAAATTACATATACT | |
| ACAGTCAAGATAGAAAGGGTGCTGCCACGGAGTGGTGAGGTACCGTCTACATTTCACATTATTCTGGGCACCACAAAATA | |
| TACAAAACTTTATCAGGGAAACTAAGATTCTTTTAAATTAGAAAATATTCTCTATTTGAATTATTTCTCTCACAGTAAAA | |
| Primer | |
| ATAAAATACTTTGAGTTTTGAGCTACTGGATTCTTATTAGTTCCCCAAATACAAAGTTAGAGAACTAAACTAGTTTTTCC | |
| Probe Primer | |
| TATCATGTTAACCTCTGCTTTTATCTCAGATGTTAAAATAAATGGTTTGGTGCTTTTTATAAAAAGATAATCTCAGTGCT | |
| TTCCTCCTTCACTGTTTCATCTAAGTGCCTCACATTTTTTTCTACCTATAACACTCTAGGATGTATATTTTATATAAAGT | |
| ATTCTTTTTCTTTTTTAAATTAATATCTTTCTGCACACAAATATTATTTGTGTTTCCTAAATCCAACCATTTTCATTAAT | |
| TCAGGCATATTTTAACTCCACTGCTTACCTACTTTCTTCAGGTAAAGGGCAAATAATGATCGAAAAAATAATTATTTATT | |
| ACATAATTTAGTTGTTTCTAGACTATAAATGTTGCTATGTGCCTTATGTTGAAAAAATTTAAAAGTAAAATGTCTTTCCA | |
| AATTATTTCTTAATTATTATAAAAATATTAAGACAATAGCACTTAAATTCCTCAACAGTGTTTTCAGAAGAAATAAATAT | |
| ACCACTCTTTACCTTTATTGATATCTCCATGATGATAGTTGAATGTTGCAATGTGAAAAATCTGCTGTTAACTGCAACCT | |
| TGTGTATTAAATTGCAAGAAGCTTTATTTCTAGCTTTTTAATTAAGCAAAGCACCCATTTCAATGTGTATAAATTGTCTT | |
| TAAAAACTGTTTTAGACCTATAATCCTTGATAATATATTGTGTTGACTTTATAAATTTCGCTTCTTAGAACAGTGGAAAC | |
| TATGTGTTTTTCTCATATTTGAGGAGTGTTAAGATTGCAGATAGCAAGGTTTGGTGCAAAGTATTGTAATGAGTGAATTG | |
| AATGGTGCATTGTATAGATATAATGAACAAAATTATTTGTAAGATATTTGCAGTTTTTCATTTTAAAAAGTCCATACCTT | |
| ATATATGCACTTAATTTGTTGGGGCTTTACATACTTTATCAATGTGTCTTTCTAAGAAATCAAGTAATGAATCCAACTGC | |
| TTAAAGTTGGTATTAATAAAAAGACAACCACATAGTTCGTTTACCTTCAAACTTTAGGTTTTTTTAATGATATACTGATC | |
| TTCATTACCAATAGGCAAATTAATCACCCTACCAACTTTACTGTCCTAACATGGTTTAAAAGAAAAAATGACACCATCTT | |
| TTATTCTTTTTTTTTTTTTTTTTGAGAGAGAGTCTTACTCTGCCGCCCAAACTGGAGTGCAGTGGCACAATCTTGGCTCA | |
| CTGCAACCTCTACCTCCTGGGTTCAAGTGATTCTCTTGCCTCAGCCTCCCGAGTTGCTGGGATTGCGGGCATGGTGGCGT | |
| GAGCCTGTAGTCCTAGCTACTCGGGAGGCTGAGGCAGGAGAATAGCCTGAACCTGGGAATCGGAGGTTGCAGGGCCAAGA | |
| TCGCCCCACTGCACTCCAGCCTGGCAATAGACCGAGACTCCGTCTCCAAAAAAAAAAAAAATACAATTTTTATTTCTTTT | |
| ACTTTTTTTAGTAAGTTAATGTATATAAAAATGGCTTCGGACAAAATATCTCTGAGTTCTGTGTATTTTCAGTCAAAACT | |
| TTAAACCTGTAGAATCAATTTAAGTGTTGGAAAAAATTTGTCTGAAACATTTCATAATTTGTTTCCAGCATGAGGTATCT | |
| AAGGATTTAGACCAGAGGTCTAGATTAATACTCTATTTTTACATTTAAACCTTTTATTATAAGTCTTACATAAACCATTT | |
| TTGTTACTCTCTTCCACATGTTACTGGATAAATTGTTTAGTGGAAAATAGGCTTTTTAATCATGAATATGATGACAATCA | |
| GTTATACAGTTATAAAATTAAAAGTTTGAAAAGCAATATTGTATATTTTTATCTATATAAAATAACTAAAATGTATCTAA | |
| GAATAATAAAATCACGTTAAACCAAATACACGTTTGTCTGTATTGTTAAGTGCCAAACAAAGGATACTTAGTGCACTGCT | |
| ACATTGTGGGATTTATTTCTAGATGATGTGCACATCTAAGGATATGGATGTGTCTAATTTTAGTCTTTTCCTGTACCAGG | |
| TTTTTCTTACAATACCTGAAGACTTACCAGTATTCTAGTGTATTATGAAGCTTTCAACATTACTATGCACAAACTAGTGT | |
| TTTTCGATGTTACTAAATTTTAGGTAAATGCTTTCATGGCTTTTTTCTTCAAAATGTTACTGCTTACATATATCATGCAT | |
| AGATTTTTGCTTAAAGTATGATTTATAATATCCTCATTATCAAAGTTGTATACAATAATATATAATAAAATAACAAATAT | |
| GAATAATAAAAAAAAAAAAAAAAA | |

Human Aryl Hydrocarbon Receptor and associated gene products function in the etiology and pathogenesis of obesity and/or diabetes. The scheme incorporates the unique findings of these discovery studies in conjunction with what has been reported in the literature. The outcome of inhibiting the action of the human Aryl Hydrocarbon Receptor would be a reduction of Insulin Resistance, a major problem in obesity and/or diabetes.

Rationale for Use as a Diagnostic and/or Target for Small Molecule Drugs and Antibody Therapeutics

The following is a summary of the findings from the discovery studies, supplementary investigations and assays that also incorporates knowledge in the scientific literature. Taken in total, the data indicates that an inhibitor/antagonist of the human Aryl Hydrocarbon Receptor would be beneficial in the treatment of obesity and/or diabetes:

a) Aryl Hydrocarbon was upregulated 1.9 fold in sub-cutaneous adipose from gestational diabetics. TCDD, an AHR agonist, suppresses PPAR-γ. Conversely TZDs activate PPAR-γ.

b) AHR activation decreases GLUT4 expression in adipose.

c) The clinical rise may represent a compensatory response.

d) No dysregulation of toxification genes (CYP1A1, CYP1A2, or CYP1B).

e) Upregulated in obese, hyperglycemic mouse liver and adipose. AHR nuclear translocator (ARNT) and AHR interacting protein (AIP) are also upregulated.

Expression analysis was performed as described in Example C.

O. Human Neutral Amino Acid Transporter B-Like Protein—CG96736-01 Discovery Process

The following sections describe the study design(s) and the techniques used to identify the Human Neutral Amino Acid Transporter B-encoded protein and any variants, thereof, as being suitable as diagnostic markers, targets for an antibody therapeutic and targets for a small molecule drugs for Obesity and Diabetes.

| | | |
|---|---|---|
| Studies: | MB.04 | Obese vs Lean Mice (Genetic) |

Study Statement: A number of genetic models of obesity have been studied, most prominently in mouse and rat, but only a few causative genes have been identified. Polygenic mouse models of obesity have been evaluated by GeneCalling in order to identify the set of genes differentially expressed in obese vs. lean animals. This strategy should lead to the discovery of drug targets for the prevention and/or treatment of obesity.

Species #1 Mouse—Strains: AKR and C57BL/6J

Human Neutral Amino Acid Transporter B: This is a Na+-dependent neutral amino acid transporter that exhibits high affinity electroneutral uptake of neutral amino acids such as L-alanine, L-serine, L-threonine, L-cysteine and L-glutamine. This transporter prefers neutral amino acids without bulky or branched side chains. It is localized to the plasma membrane and has eight putative transmembrane segments. It appears to be a Type IIIa membrane protein with an N-terminal cytoplasmic tail and a C-terminal extracellular segment. A connection between this transporter and obesity and/or diabetes has not previously been reported.

SPECIES #1—A gene fragment of the mouse Neutral Amino Acid Transporter B was initially found to be up-regulated by 6 fold in the adipose tissue of obese mice (AKR) relative to non-obese mice (C57BL/6J) using CuraGen's GeneCalling™ method of differential gene expression. Two differentially expressed mouse gene fragments migrating, at approximately 138 and 347 nucleotides in length (FIGS. 1A, 1B for Sequence 1A, and FIGS. 1C and 1D for Sequence 1B respectively—vertical line) were definitively identified as a component of the Mouse Neutral Amino Acid Transporter B cDNA (in the graphs, the abscissa is measured in lengths of nucleotides and the ordinate is measured as signal response). The method of competitive PCR was used for conformation of the gene assessment. The electropherogramatic peaks corresponding to the gene fragment of the mouse Neutral Amino Acid Transporter B are ablated when a gene-specific primer (see below) competes with primers in the linker-adaptors during the PCR amplification. The peaks at 138 nt length are ablated in the sample from both the obese and non-obese mice.

The direct sequences of the 138.4 and 346.7 nucleotide-long gene fragments and the gene-specific primers used for competitive PCR are indicated on the cDNA sequence of the Mouse Neutral Amino Acid Transporter B are shown below in bold. The gene-specific primers at the 5′ and 3′ ends of the fragment are in italics. Competitive PCR Primer for the Mouse Neutral Amino Acid Transporter B (peak at 138.4) is below.

TABLE O1
Sequence #1A Gene Sequence

(fragment from 564 to 700 in bold. band size: 137)

(SEQ ID NO:678)

CCAGAGAGGACCAGAGTGCGAAAGCAGGTGGTTGCTGCGGTTCCCGTGACCGGGTGCGCCGCTGCATTCGCGCCAACCTG
CTGGTGCTGCTCACGGTGGCTGCGGTGGTGGCTGGCGTGGGGCTGGGGCTGGGGGTCTCGGCGGCGGGCGGTGCTGACGC
GCTGGGTCCCGCGCGCTTGACCGCTTTCGCCTTCCCGGGAGAGCTGCTGCTGCGTCTGCTGAAGATGATCATCCTGCCGC
TCGTGGTGTGCAGCCTGATCGGAGGTGCAGCCAGCTTGGACCCTAGCGCGCTCGGTCGTGTGGGCGCCTGGGCGCTGCTC
TTTTTCCTGGTCACCACACTGCTCGCGTCGGCGCTCGGCGTGGGTTTGGCCCTGGCGCTGAAGCCGGGCGCCGCCGTTAC
CGCCATCACCTCCATCAACGACTCTGTTGTAGACCCCTGTGCCCGCAGTGCACCAACCAAAGAGGTGCTGGATTCCTTTC
TAGATCTCGTGAGGAATATTTTCCCCTCCAATCTGGTGTCTGCTGCCTTCCGCTCTTTTGCTACCTCATATGAACCCAAA
GACAACTCATGTAAAATACCGCAATCCTGTATCCAGCGGGAGATAAATTCAACCATGGTCCAGCTTCTCTGTCAGCTGGA
GGGAATGAACATCCTGGGCCTGGTGGTCTTCGCTATCGTCTTTCGTGTGGCTCTGCGGAAGCTGGGGCCCGAGGGTGAGC
TGCTCATTCGTTTCTTCAACTCCTTCAATGATGCCACCATGGTCCTGGTCTCCTGGATTATGTGGTACGCACCCGTTGGA
ATCCTGTTCCTGGTGGCCAGCAAGATTGTGGAGATGAAAGACGTCCGCCAGCTCTTCATCAGCCTCGGCAAATACATTCT
GTGCTGCCTGCTGGGCCACGCCATCCACGGGCTCCTGGTTCTGCCTCTCATCTACTTCCTCTTCACCCGCAAAAATCCCT
ATCGATTCCTGTGGGGCATCATGACACCCCTGOCCACTGCTTTCGCGACCTCTTCTAGCTCTGCCACCTTGCCTCTGATG
ATGAAGTGTGTAGAGGAGAAGAATCGTGTGGCCAAACACATCAGCCGGTTCATCCTAC
(gene length is 1668, only region from 83 to 1180 shown)

Competitive PCR Primer for the Mouse Neutral Amino Acid Transporter B (peak at 346.7): (The gene-specific primers at the 5′ and 3′ ends of the fragment are in italics.)

TABLE O2

Sequence #1B Gene Sequence
(fragment from 1 to 347 in italics, band size: 347)

(SEQ ID NO:679)

GGATCCCTGCCGCACCGACACTGGATGCTGTGGCTGTGACCCTGGGGAAGAGAAGAGCGGAGATGGCAGAATCATGGGGG
CGGGGCCTCCTGCCACAGCCCCTGGCACTCACACGATGGTGATGATCTTCACGAAGTCCAGGGACACCCCGTTTAGTTGT
GCGATGAACACTGCCGCCACACACTGGAACAGCGCCGCCCCGTCCATGTTGACCGTGGCGCCGATGGGTAGGATGAACCG
GCTGATGTGTTTGGCCACACCATTCTTCTCCTCTACACACTTCATCATCAGAGGCAAGGTGGCAGAGCTAGAAGAGGTCC
CGAAAGCAGTGGCCAGGGGTGTCATGA
(gene length is 347, only region from 1 to 347 shown)

Tables O3 shows differentially expressed mouse neutral amino acid transporter B gene fragment, Sequence #1A, from Discovery Study MB.04, and Table O4 shows differentially expressed mouse neutral amino acid transporter B gene fragment, Sequence #1B, from Discovery Study MB.04.

[image]

TABLE O5
Human Neutral Amino Acid Transporter B Gene Sequence

>CG96736-01 2885 nt

(SEQ ID NO:680)

CGGCACGCCCGGGAGGCTTTCTCTGGCTGGTAACCGCTACTCCCGGACACCAGACCACCGCCTTCCGTACACAGGGGCCC
GCATCCCACCCTCCCGGACCTAAGAGCCTGGGTCCCCTGTTTCCGGAGTCCGCTTCCCGGCCCCCAGATTCTGGCATCCC
AGCCCTCAGTGTCCAAGACCCAGGCAGCCCGGGTCCCCGCCTCCCGGATCCAGGCGTCCGGGATCTGCGCCACCAGAACC
TAGCCTCCTGCAGACCTCCGCCATCTGGGGGCACTCAACCTCCTGGAGCCAAGGGCCCCACGTCCCACCCAGAGAAACTC
TCGTATTCCCAGCTCCTAGGGCCAAGGAACCCGGGCGCTCCGAACTCCCAGCTTTCGGACATCTGGCACACGGGGCAGAG
CAGAGAAGCCTCAGCGCCCAGCCTGGGGAATTTAAACACTCCAGCTTCCAAGAGCCAAGGAACTTCAGTGCTGTGAACTC
ACAACTCTAAGGAGCCCTCCAAAGTTCCAGTCTCCAGGTGCTGTTACTCAACTCAGTCCTAGGAACGTCGGGTCCTGGGA
AGGAGCCCAAGCGCTCCCAGCCAGCTTCCAGGCGCTAAGAAACCCCGGTGCTTCCCATCATGGTGGCCGATCCTCCTCGA
GACTCCAAGGGGCTCGCAGCGGCGGAGCCACCGCCAACGGGGGCCTGGCAGCTGGCCTCCATCGAGGACCAAGGCGCGGC
AGCAGGCGGCTACTGCGGTTCCCGGGACCTGGTGCGCCGCTGCCTTCGAGCCAACCTGCTTGTGCTGCTGACAGTGGTGG
CCGTGGTGGCCGGCGTGGCGCTGGGACTGGGGGTGTCGGGGGCCGGGGGTGCGCTGGCGTTGGGCCCGGGAGCGCTTGAG
GCCTTCGTCTTCCCGGGCGAGCTGCTGCTGCGTCTGCTGCGGATGATCATCTTGCCGCTGGTGGTGTGCAGCTTGATCGG
CGGCGCCGCCAGCCTGGACCCCGGCGCGCTCGGCCGTCTGGGCGCCTGGGCGCTGCTCTTTTTCCTGGTCACCACGCTGC
TGGCGTCGGCGCTCGGAGTGGGCTTGGCGCTGGCTCTGCAGCCGGGCGCCGCCTCCGCCGCCATCAACGCCTCCGTGGGA
GCCGCGGGCAGTGCCGAAAATGCCCCCAGCAAGGAGGTGCTCGATTCGTTCCTGGATCTTGCGAGAAATATCTTCCCTTC
CAACCTGGTGTCAGCAGCCTTTCGCTCATACTCTACCACCTATGAAGAGAGGAATATCACCGGAACCAGGGTGAAGGTGC
CCGTGGGGCAGGAGGTGGAGGGGATGAACATCCTGGGCTTGGTAGTGTTTGCCATCGTCTTTGGTGTGGCGCTGCGGAAG
CTGGGGCCTGAAGGGGAGCTGCTTATCCGCTTCTTCAACTCCTTCAATGAGGCCACCATGGTTCTGGTCTCCTGGATCAT
GTGGTACGCCCCTGTGGGCATCATGTTCCTGGTGGCTGGCAAGATCGTGGAGATGGAGGATGTGGGTTTACTCTTTGCCC
GCCTTGGCAAGTACATTCTGTGCTGCCTGCTGGGTCACGCCATCCATGGGCTCCTGGTACTGCCCCTCATCTACTTCCTC
TTCACCCGCAAAAACCCCTACCGCTTCCTGTGGGGCATCGTGACGCCGCTGGCCACTGCCTTTGGGACCTCTTCCAGTTC
CGCCACGCTGCCGCTGATGATGAAGTGCGTGGAGGAGAATAATGGCGTGGCCAAGCACATCAGCCGTTTCATCCTGCCCA
TCGGCGCCACCGTCAACATGGACGGTGCCGCGCTCTTCCAGTGCGTGGCCGCAGTGTTCATTGCACAGCTCAGCCAGCAG
TCCTTGGACTTCGTAAAGATCATCACCATCCTGGTCACGGCCACAGCGTCCAGCGTGGGGGCAGCGGGCATCCCTGCTGG
AGGTGTCCTCACTCTGGCCATCATCCTCGAAGCAGTCAACCTCCCGGTCGACCATATCTCCTTGATCCTGGCTGTGGACT
GGCTAGTCGACCGGTCCTGTACCGTCCTCAATGTAGAAGGTGACGCTCTGGGGGCAGGACTCCTCCAAAATTATGTGGAC
CGTACGGAGTCGAGAAGCACAGAGCCTGAGTTGATACAAGTGAAGAGTGAGCTGCCCCTGGATCCGCTGCCAGTCCCCAC
TGAGGAAGGAAACCCCCTCCTCAAACACTATCGGGGGCCCGCAGGGGATGCCACGGTCGCCTCTGAGAAGGAATCAGTCA
TGTAAACCCCGGGAGGGACCTTCCCTGCCCTGCTGGGGGTGCTCTTTGGACACTGGATTATGAGGAATGGATAAATGGAT
GAGCTAGGGCTCTGGGGGTCTGCCTGCACACTCTGGGGAGCCAGGGGCCCCAGCACCCTCCAGGACAGGAGATCTGGGAT
GCCTGGCTGCTGGAGTACATGTGTTCACAAGGGTTACTCCTCAAAACCCCCAGTTCTCACTCATGTCCCCAACTCAAGGC
TAGAAAACAGCAAGATGGAGAAATAATGTTCTGCTGCGTCCCCACCGTGACCTGCCTGGCCTCCCCTGTCTCAGGGAGCA
GGTCACAGGTCACCATGGGGAATTCTAGCCCCCACTGGGGGGATGTTACAACACCATGCTGGTTATTTTGGCGGCTGTAG
TTGTGGGGGGATGTGTGTGTGCACGTGTGTGTGTGTGTGTGTGTGTGTGTGTGTGTGTGTTCTGTGACCTCCTGTCCCCA
TGGTACGTCCCACCCTGTCCCCAGATCCCCTATTCCTCCCACAATAACAGAAACACTCCCAGGGACTCTGGGGAGAGGCT
GAGGACAAATACCTGCTGTCACTCCAGAGGACATTTTTTTTAGCAATAAAATTGAGTGTCAACTATTAAAAAAAAAAAAA
AAAAA

TABLE O6
Human Neutral Amino Acid Transporter B Protein Sequence

ORF Start: 620 ORF Stop: 2243 Frame: 2
>CG96736-01-prot 541 aa

(SEQ ID NO:681)

MVADPPRDSKGLAAAEPPPTGAWQLASIEDQGAAAGGYCGSRDLVRRCLRANLLVLLTVVAVVAGVALGLGVSGAGGALA
LGPGALEAFVFPGELLLRLLEMIILPLVVCSLIGGAASLDPGALGRLGAWALLFFLVTTLLASALGVGLALALQPGAASA
AINASVGAAGSAENAPSKEVLDSFLDLARNIFPSNLVSAAFRSYSTTYEERNITGTRVKVPVGQEVEGMNILGLVVFAIV
FGVALRKLGPEGELLIRFFNSFNEATMVLVSWIMWYAPVGIMFLVAGKIVEMEDVGLLFARLGKYILCCLLGHAIHGLLV
LPLIYFLFTRKNPYRFLWGIVTPLATAFGTSSSSATLPLMMKCVEENNGVAKHISRFILPIGATVNMDGAALFQCVAAVF
IAQLSQQSLDFVKIITILVTATASSVGAAGIPAGGVLTLAIILEAVNLPVDHISLILAVDWLVDRSCTVLNVEGDALGAG
LLQNYVDRTESRSTEPELIQVKSELPLDPLPVPTEEGNPLLKHYRGPAGDATVASEKESVM

The following is an alignment of the protein sequences of the human (CG96736-01) and mouse versions of the Neutral Amino Acid Transporter B: 80% overall homology.

[image]

In addition to the human version of the neutral amino acid transporter B identified as being differentially expressed in the experimental study, no other variant has been identified by direct sequencing of cDNAs derived from many different human tissues and from sequences in public databases. No splice-form variants have been identified at CuraGen. See the table below for SNPs identified. The preferred variant of all those identified, to be used for screening purposes, is CG96736-01.

TABLE O8

The variants of the Human Neutral Amino Acid
Transporter B obtained from direct cloning
and/or public databases.

| | | | | |
|---|---|---|---|---|
| DNA Position | Strand | Alleles | AA Position | AA Change |

| | | | | |
|---|---|---|---|---|
| 272 | Plus | C:T | 0 | N/A => N/A |
| 281 | Plus | T:C | 0 | N/A => N/A |
| 1484 | Plus | A:G | 289 | Ile => Val |
| 2021 | Plus | A:G | 468 | Thr => Ala |
| 2036 | Plus | G:A | 473 | Glu => Lys |
| 2074 | Minus | C:T | 485 | Tyr => Tyr |
| 2074 | Minus | C:T | 485 | Tyr => Tyr |
| 2153 | Plus | G:C | 512 | Val => Leu |
| 2157 | Plus | C:T | 513 | Pro => Leu |
| 2160 | Plus | C:T | 514 | Thr => Ile |
| 2329 | Plus | G:A | 0 | N/A => N/A |

Table O9. RTQ-PCR Human Expression Profiles

Quantitative expression analysis of clones in various cells and tissues was performed as described in Example C.

CG96736-01: Neutral amino acid transporter B—isoform 1

Expression of gene CG96736-01 was assessed using the primer-probe set Ag4075, described in Tables OAA and. Results of the RTQ-PCR runs are shown in Tables OAC, OAD and OAF.

TABLE OAA

Probe Name Ag4075

| | | | | |
|---|---|---|---|---|
| | | | Start | SEQ ID |
| Primers | Sequences | Length | Position | NO |

| | | | | |
|---|---|---|---|---|
| Forward | 5′-cgagaaatatcttcccttccaa-3′ | 22 | 1182 | 441 |
| Probe | TET-5′-tgtcagcagcctttcgctcatactct-3′-TAMRA | 26 | 1209 | 442 |
| Reverse | 5′-ttccggtgatattcctctcttc-3′ | 22 | 1244 | 443 |

TABLE OAC
General_screening_panel_v1.4

| | Rel. | Rel. |
|---|---|---|
| | Exp. (%) | Exp. (%) |
| | Ag4075, | Ag4075, |
| | Run | Run |
| Tissue Name | 212696066 | 218525356 |
| Adipose | 0.0 | 1.3 |
| Melanoma* Hs688(A).T | 14.4 | 23.2 |
| Melanoma* Hs688(B).T | 19.1 | 29.9 |
| Melanoma* M14 | 9.5 | 12.7 |
| Melanoma* LOXIMVI | 8.1 | 12.9 |
| Melanoma* SK-MEL-5 | 5.9 | 14.2 |
| Squamous cell carcinoma SCC-4 | 5.1 | 10.2 |
| Testis Pool | 1.4 | 1.9 |
| Prostate ca.* (bone met) PC-3 | 9.5 | 13.6 |
| Prostate Pool | 1.1 | 1.5 |
| Placenta | 1.1 | 1.3 |
| Uterus Pool | 0.1 | 0.2 |
| Ovarian ca. OVCAR-3 | 6.5 | 8.0 |
| Ovarian ca. SK-OV-3 | 8.1 | 9.9 |
| Ovarian ca. OVCAR-4 | 9.2 | 16.4 |
| Ovarian ca. OVCAR-5 | 28.1 | 32.1 |
| Ovarian ca. IGROV-1 | 23.0 | 33.2 |
| Ovarian ca. OVCAR-8 | 10.3 | 16.4 |
| Ovary | 0.5 | 0.8 |
| Breast ca. MCF-7 | 15.7 | 17.2 |
| Breast ca. MDA-MB-231 | 10.4 | 15.6 |
| Breast ca. BT 549 | 9.9 | 18.7 |
| Breast ca. T47D | 53.2 | 51.8 |
| Breast ca. MDA-N | 4.7 | 6.3 |
| Breast Pool | 0.6 | 0.6 |
| Trachea | 3.6 | 5.3 |
| Lung | 0.1 | 0.1 |
| Fetal Lung | 2.4 | 4.0 |
| Lung ca. NCI-N417 | 1.6 | 0.0 |
| Lung ca. LX-1 | 81.8 | 82.4 |
| Lung ca. NCI-H146 | 0.4 | 0.8 |
| Lung ca. SHP-77 | 6.8 | 8.5 |
| Lung ca. A549 | 9.8 | 15.8 |
| Lung ca. NCI-H526 | 2.1 | 2.5 |
| Lung ca. NCI-H23 | 4.3 | 4.2 |
| Lung ca. NCI-H460 | 9.2 | 16.2 |
| Lung ca. HOP-62 | 4.4 | 4.5 |
| Lung ca. NCI-H522 | 9.5 | 10.0 |
| Liver | 0.0 | 0.1 |
| Fetal Liver | 2.9 | 4.3 |
| Liver ca. HepG2 | 6.7 | 7.9 |
| Kidney Pool | 1.1 | 1.2 |
| Fetal Kidney | 0.3 | 0.5 |
| Renal ca. 786-0 | 5.1 | 9.5 |
| Renal ca. A498 | 3.1 | 5.0 |
| Renal ca. ACHN | 5.1 | 5.9 |
| Renal ca. UO-31 | 2.6 | 4.2 |
| Renal ca. TK-10 | 9.7 | 14.8 |
| Bladder | 1.0 | 1.8 |
| Gastric ca. (liver met.) NCI-N87 | 41.5 | 42.0 |
| Gastric ca. KATO III | 25.5 | 22.8 |
| Colon ca. SW-948 | 4.4 | 5.6 |
| Colon ca. SW480 | 100.0 | 100.0 |
| Colon ca.* (SW480 met) SW620 | 41.5 | 50.0 |
| Colon ca. HT29 | 10.2 | 13.6 |
| Colon ca. HCT-116 | 13.0 | 20.9 |
| Colon ca. CaCo-2 | 12.0 | 14.5 |
| Colon cancer tissue | 5.0 | 8.4 |
| Colon ca. SW1116 | 14.7 | 15.9 |
| Colon ca. Colo-205 | 24.7 | 29.5 |
| Colon ca. SW-48 | 3.6 | 4.7 |
| Colon Pool | 0.7 | 1.1 |
| Small Intestine Pool | 0.5 | 0.6 |
| Stomach Pool | 0.8 | 0.8 |
| Bone Marrow Pool | 0.2 | 0.4 |
| Fetal Heart | 0.1 | 0.1 |
| Heart Pool | 0.2 | 0.3 |
| Lymph Node Pool | 1.2 | 1.0 |
| Fetal Skeletal Muscle | 0.2 | 0.2 |
| Skeletal Muscle Pool | 0.2 | 0.3 |
| Spleen Pool | 0.7 | 0.5 |
| Thymus Pool | 0.8 | 0.9 |
| CNS cancer (glio/astro) U87-MG | 20.0 | 20.3 |
| CNS cancer (glio/astro) U-118-MG | 11.2 | 12.9 |
| CNS cancer (neuro; met) SK-N-AS | 6.9 | 8.9 |
| CNS cancer (astro) SF-539 | 9.3 | 12.0 |
| CNS cancer (astro) SNB-75 | 36.1 | 55.5 |
| CNS cancer (glio) SNB-19 | 30.1 | 37.6 |
| CNS cancer (glio) SF-295 | 58.6 | 60.7 |
| Brain (Amygdala) Pool | 0.0 | 0.1 |
| Brain (cerebellum) | 0.1 | 0.2 |
| Brain (fetal) | 0.2 | 0.3 |
| Brain (Hippocampus) Pool | 0.1 | 0.1 |
| Cerebral Cortex Pool | 0.0 | 0.1 |
| Brain (Substantia nigra) Pool | 0.1 | 0.1 |
| Brain (Thalamus) Pool | 0.0 | 0.1 |
| Brain (whole) | 0.2 | 0.2 |
| Spinal Cord Pool | 0.2 | 0.3 |
| Adrenal Gland | 0.3 | 0.6 |
| Pituitary gland Pool | 0.1 | 0.3 |
| Salivary Gland | 3.0 | 2.8 |
| Thyroid (female) | 0.1 | 0.1 |
| Pancreatic ca. CAPAN2 | 7.9 | 12.2 |
| Pancreas Pool | 1.3 | 1.2 |

TABLE OAD
General_screening_panel_v1.5

| | Rel. | | Rel. |
|---|---|---|---|
| | Exp. (%) | | Exp. (%) |
| | Ag4075, | | Ag4075, |
| | Run | | Run |
| Tissue Name | 228714883 | Tissue Name | 228714883 |
| Adipose | 1.0 | Renal ca. TK-10 | 9.8 |
| Melanoma* Hs688(A).T | 18.0 | Bladder | 1.4 |
| Melanoma* Hs688(B).T | 17.4 | Gastric ca. (liver met.) NCI-N87 | 35.4 |
| Melanoma* M14 | 9.5 | Gastric ca. KATO III | 19.9 |
| Melanoma* LOXIMVI | 9.0 | Colon ca. SW-948 | 4.4 |
| Melanoma* SK-MEL-5 | 8.7 | Colon ca. SW480 | 100.0 |
| Squamous cell carcinoma SCC-4 | 5.8 | Colon ca.* (SW480 met) SW620 | 32.8 |
| Testis Pool | 1.2 | Colon ca. HT29 | 9.9 |
| Prostate ca.* (bone met) PC-3 | 10.8 | Colon ca. HCT-116 | 15.2 |
| Prostate Pool | 1.5 | Colon ca. CaCo-2 | 11.1 |
| Placenta | 1.1 | Colon cancer tissue | 5.1 |
| Uterus Pool | 0.3 | Colon ca. SW1116 | 7.2 |
| Ovarian ca. OVCAR-3 | 6.2 | Colon ca. Colo-205 | 23.7 |
| Ovarian ca. SK-OV-3 | 7.5 | Colon ca. SW-48 | 3.2 |
| Ovarian ca. OVCAR-4 | 12.5 | Colon Pool | 0.7 |
| Ovarian ca. OVCAR-5 | 20.2 | Small Intestine Pool | 0.4 |
| Ovarian ca. IGROV-1 | 23.8 | Stomach Pool | 0.7 |
| Ovarian ca. OVCAR-8 | 11.2 | Bone Marrow Pool | 0.2 |
| Ovary | 0.6 | Fetal Heart | 0.1 |
| Breast ca. MCF-7 | 14.4 | Heart Pool | 0.2 |
| Breast ca. MDA-MB-231 | 14.1 | Lymph Node Pool | 0.7 |
| Breast ca. BT 549 | 8.4 | Fetal Skeletal Muscle | 0.2 |
| Breast ca. T47D | 2.1 | Skeletal Muscle Pool | 0.4 |
| Breast ca. MDA-N | 3.6 | Spleen Pool | 0.3 |
| Breast Pool | 0.5 | Thymus Pool | 0.5 |
| Trachea | 4.6 | CNS cancer (glio/astro) U87-MG | 12.5 |
| Lung | 0.1 | CNS cancer (glio/astro) U-118-MG | 8.5 |
| Fetal Lung | 2.6 | CNS cancer (neuro; met) SK-N-AS | 5.5 |
| Lung ca. NCI-N417 | 1.9 | CNS cancer (astro) SF-539 | 8.4 |
| Lung ca. LX-1 | 81.8 | CNS cancer (astro) SNB-75 | 13.1 |
| Lung ca. NCI-H146 | 0.6 | CNS cancer (glio) SNB-19 | 27.2 |
| Lung ca. SHP-77 | 7.7 | CNS cancer (glio) SF-295 | 53.2 |
| Lung ca. A549 | 11.8 | Brain (Amygdala) Pool | 0.0 |
| Lung ca. NCI-H526 | 2.1 | Brain (cerebellum) | 0.1 |
| Lung ca. NCI-H23 | 3.5 | Brain (fetal) | 0.2 |
| Lung ca. NCI-H460 | 8.8 | Brain (Hippocampus) Pool | 0.0 |
| Lung ca. HOP-62 | 3.5 | Cerebral Cortex Pool | 0.1 |
| Lung ca. NCI-H522 | 7.5 | Brain (Substantia nigra) Pool | 0.1 |
| Liver | 0.0 | Brain (Thalamus) Pool | 0.1 |
| Fetal Liver | 2.9 | Brain (whole) | 0.2 |
| Liver ca. HepG2 | 6.2 | Spinal Cord Pool | 0.1 |
| Kidney Pool | 0.8 | Adrenal Gland | 0.4 |
| Fetal Kidney | 0.3 | Pituitary gland Pool | 0.2 |
| Renal ca. 786-0 | 5.6 | Salivary Gland | 2.7 |
| Renal ca. A498 | 3.4 | Thyroid (female) | 0.1 |
| Renal ca. ACHN | 4.9 | Pancreatic ca. CAPAN2 | 9.7 |
| Renal ca. UO-31 | 2.4 | Pancreas Pool | 0.8 |

TABLE OAE
Panel 5 Islet

| | Rel. | | Rel. |
|---|---|---|---|
| | Exp. (%) | | Exp. (%) |
| | Ag4075, | | Ag4075, |
| | Run | | Run |
| Tissue Name | 186511155 | Tissue Name | 186511155 |
| 97457_Patient-02go_adipose | 7.6 | 94709_Donor 2 AM - A_adipose | 45.7 |
| 97476_Patient-07sk_skeletal | 2.9 | 994710_Donor 2 AM - B_adipose | 27.4 |
| muscle | | | |
| 97477_Patient-07ut_uterus | 3.5 | 94711_Donor 2 AM - C_adipose | 15.2 |
| 97478_Patient-07pl_placenta | 5.0 | 94712_Donor 2 AD - A_adipose | 62.9 |
| 99167_Bayer_Patient 1 | 30.6 | 94713_Donor 2 AD - B_adipose | 66.4 |
| 97482_Patient-08ut_uterus | 4.6 | 94714_Donor 2 AD - C_adipose | 57.4 |
| 97483_Patient-08pl_placenta | 3.8 | 94742_Donor 3 U - A_Mesenchymal | 36.1 |
| | | Stem Cells | |
| 97486_Patient-09sk_skeletal | 0.3 | 94743_Donor 3 U - B_Mesenchymal | 62.4 |
| muscle | | Stem Cells | |
| 97487_Patient-09ut_uterus | 8.3 | 94730_Donor 3 AM - A_adipose | 34.9 |
| 97488_Patient-09pl_placenta | 3.4 | 94731_Donor 3 AM - B_adipose | 17.2 |
| 97492_Patient-10ut_uterus | 7.5 | 94732_Donor 3 AM - C_adipose | 22.4 |
| 97493_Patient-10pl_placenta | 5.1 | 94733_Donor 3 AD - A_adipose | 100.0 |
| 97495_Patient-11go_adipose | 6.4 | 94734_Donor 3 AD - B_adipose | 32.3 |
| 97496_Patient-11sk_skeletal | 1.3 | 94735_Donor 3 AD - C_adipose | 66.9 |
| muscle | | | |
| 97497_Patient-11ut_uterus | 11.6 | 77138_Liver_HepG2untreated | 31.4 |
| 97498_Patient-11pl_placenta | 3.9 | 73556_Heart_Cardiac stromal cells | 3.6 |
| | | (primary) | |
| 97500_Patient-12go_adipose | 8.5 | 81735_Small Intestine | 6.4 |
| 97501_Patient-12sk_skeletal | 2.7 | 72409_Kidney_Proximal Convoluted | 3.8 |
| muscle | | Tubule | |
| 97502_Patient-12ut_uterus | 8.7 | 82685_Small intestine_Duodenum | 1.9 |
| 97503_Patient-12pl_placenta | 3.1 | 90650_Adrenal_Adrenocortical | 1.4 |
| | | adenoma | |
| 94721_Donor 2 U - | 40.1 | 72410_Kidney_HRCE | 14.9 |
| A_Mesenchymal Stem Cells | | | |
| 94722_Donor 2 U - | 23.7 | 72411_Kidney_HRE | 11.1 |
| B_Mesenchymal Stem Cells | | | |
| 94723_Donor 2 U - | 52.5 | 73139_Uterus_Uterine smooth | 17.4 |
| C_Mesenchymal Stem Cells | | muscle cells | |

Rationale for Use as a Diagnostic and/or Target for Small Molecule Drugs and Antibody Therapeutics.

The following is a summary of the findings from the discovery studies, supplementary investigations and assays that also incorporates knowledge in the scientific literature. Taken in total, the data indicates that an inhibitor/antagonist of the Human Neutral Amino Acid Transporter B would be beneficial in the treatment of obesity and/or diabetes.

The pathophysiologic basis of obesity in the AKR mouse is not known. Neutral amino acid transporter B (NATB) is upregulated 6-fold in adipose tissue of obese AKR versus normal C57L mice. NATB transports the gluconeogenic amino acids L-alanine and L-glutamine across the plasma membrane into the cell. Phosphoenolpyruvate carboxykinase, the rate-limiting gluconeogenic enzyme, is also increased 3-fold in adipose tissue of AKR versus C57L mice. Thus, excess neutral amino acid transport and glucose production may lead to increased triglyceride synthesis in adipose tissue, resulting in obesity in the AKR mouse. The data from this genetic comparison indicates that inhibition of NATB may be an effective treatment for the prevention of obesity in human populations.

Methods of Use for the Compositions of the Invention

The protein similarity information, expression pattern, cellular localization, and map location for the protein and nucleic acid disclosed herein suggest that this protein may have important structural and/or physiological functions characteristic of the Human Neutral Amino Acid Transporter B family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed.

The nucleic acids and proteins of the invention have applications in the diagnosis and/or treatment of various diseases and disorders. For example, the compositions of the present invention will have efficacy for the treatment of patients suffering from: obesity and/or diabetes. These materials are further useful in the generation of antibodies that bind immunospecifically to the substances of the invention for use in diagnostic and/or therapeutic methods.

P. Human Cytosolic HMG CoA Synthase-Like Protein—CG97025-01

The following sections describe the study design(s) and the techniques used to identify the Cytosolic HMG CoA synthase-encoded protein and any variants, thereof, as being suitable as diagnostic markers, targets for an antibody therapeutic and targets for a small molecule drugs for obesity and/or diabetes.

Studies:MB.04: Mean vs. Obese Genetic mouse model

MB.04 A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

Species #1 Mouse Strains NZB vs SMJ

Cytoplasmic HMG CoA synthase mediates an early step in cholesterol biosynthesis. This enzyme condenses acetyl-CoA with acetoacetyl-CoA to form HMG-CoA, which is the substrate for HMG-CoA Reductase.

SPECIES #1—A gene fragment of the mouse cytosolic HMG CoA synthase was initially found to be up-regulated by 7 fold in the liver of the NZB mouse relative to the SMJ mouse strain using CuraGen's GeneCalling™ method of differential gene expression. A differentially expressed mouse gene fragment migrating, at approximately 312 nucleotides in length (FIGS. 1A and 1B.—vertical line) was definitively identified as a component of the mouse Cytosolic HMG CoA synthase cDNA (in the graphs, the abscissa is measured in lengths of nucleotides and the ordinate is measured as signal response). The method of competitive PCR was used for conformation of the gene assessment. The chromatographic peaks corresponding to the gene fragment of the rat Cytosolic HMG CoA synthase are ablated when a gene-specific primer (see below) competes with primers in the linker-adaptors during the PCR amplification. The peaks at 312 nt in length are ablated in the sample from both the NZB and SMJ mice.

Competitive PCR Primer for the Human Cytosolic HMG CoA Synthase

Confirmatory Result—Human Cytosolic HMG CoA synthase (Discovery Study MB.04): The direct sequence of the 312 nucleotide-long gene fragment and the gene-specific primers used for competitive PC are indicated in italic. The gene-specific primers at the 5′ and 3′ ends of the fragment are in bold.

TABLE P1
Human Cytosolic HMG CoA synthase Gene Sequence

(Identified fragment from 101 to 412 in italic. band size: 312)

(SEQ ID NO:444)

GTATTTCTGTGTTTTGTTTGTTTTTGTATCCGTTCGAAAATTTAACCCACATTTTCACATAGTGAAAATTTCACATGGTCTGATTA
GCCAAAAAAGAATAAGATCTAGAAGTAGAACTCACACCATTTTTTTTCTTAACTTTGATTTCTAAAACAACAAAAACTACCACATG
AGCTGAATAAGAAAATTCACTAGCAACTTCTCTCCATGATTTTTGGTGCTGAACAATCACATCACCCTCAGACTCTAAAATACAGG
TAGTTCCAACTAATGTACAGAACTAAATTTCTTAACCTTATTTCCGTTTAATTCTCTGAAGTTTCAGTTATCTAAAATAAATGTGT
AATGTTTCAGATTGCAAGGTGATAAGTAATGTAGCATTTGTAAGATACTCTTGTCAATATTAACTAGTAGGATTTTGATTTGTACA
GTTTTAATTGGTTAAAATGATCTCATTTTAACATCCACTGCTATAGATGAATAATGTAACTTCAGATTTAATGAATGGTGGGGAGA
TGGTGCATGTAATTTTTTTGCAAGTATTGAGAGTTCTGTATGTTTTGAAAAGAGTAATTTTAACCTTTGGGTGCCAAGAAGTGGGT
TTTCTCAGAGTCCATTGCCGGCAATGGGCAAGCCTGGCGGTACTGGCACGGAGCGTTAACCACACCTTTCTAATAGCAAGGCCAAT
AACTTTGAAATAAAGTTTTAGACAAATAAAAAAAAAAAAAAAAAAAAAAAAAAAAAAAAAAAAG

TABLE P2
Nucleotide and protein sequence of Human Cytosolic HMG CoA synthase:

C097025-01

(SEQ ID NO:445)

CCTTCACACAGCTCTTTCACCATGCCTGGATCACTTCCTTTGAATGCAGAAGCTTGCTGGCCAAAAGATGTTGGGATTGTTGC
CCTTGAGATCTATTTTCCTTCTCAATATGTTGATCAAGCAGAGTTGGAAAAATATGATGGTGTAGATGCTGCGAAGTATACCA
TTGGCTTGGGCCAGGCCAAGATGGGCTTCTGCACAGATAGAGAAGATATTAACTCTCTTTGCATGACTGTGGTTCAGAATCTT
ATGGAGAGAAATAACCTTTCCTATGATTGCATTGGGCGGCTGGAAGTTGGAACAGAGACAATCATCGACAAATCAAAGTCTGT
GAAGACTAATTTGATGCAGCTGTTTGAAGAGTCTGGGAATACAGATATAGAAGGAATCGACACAACTAATCCATGCTATGGAG
GCACAGCTGCTGTCTTCAATGCTGTTAACTGGATTGAGTCCAGCTCTTGGGATGGACGGTATGCCCTGGTAGTTGCAGGAGAT
ATTGCTGTATATGCCACAGGAAATGCTAGACCTACAGGTGGAGTTGGAGCAGTAGCTCTGCTAATTGGGCCAAATGCTCCTTT
AATTTTTGAACGAGGGCTTCGTGGGACACATATGCAACATGCCTATGATTTTTACAAGCCTGATATGCTATCTGAATATCCTA
TAGTAGATCAGGAAACTCTCCATACAGTGCTACCTCAGTGCATTAGACCGCTGCTATCTGTCTACTGCAAAAAGATCCATGCC
CAGTGGCAGAAAGAGGGAAATGATAAAGATTTTACCTTGAATGATTTTGGCTTCATGATCTTTCACTCACCATATTGTAAACT
GGTTCAGAAATCTCTAGCTCGGATGTTGCTGAATGACTTCCTTAATGACCAGAATAAAGATAAAAATAGTATCTATAGTGGCC
TGGAAGCCTTTGGGGATGTTAAATTAGAAGACACCTACTTTGATAGAGATGTGGAGAAGGCATTTATGAAGGCTAGCTCTGAA
CTCTTCAGTCAGAAAACAAAGGCATCTTTACTTGTATCAAATCAAAATGGAAATATGTACACATCTTCAGTATATGGTTCCCT
TGCATCTCTTCTAGCACAGTAOTCACCTCAGCAATTAGCAGGGAAGAGAATTGGAGTGTTTTCTTATGGTTCTGGTTTGGCTG
CCACTCTGTACTCTCTTAAAGTCACACAAGATGCTACACCCGCGTCTGCTCTTGATAAAATAACAGCAACTTTATGTGATCTT
AAATCAAGGCTTGATTCAAGAACTGGTGTGGCACCAGATCTCTTCGCTGAAAACATGAAGCTCAGAGAGGACACCCATCATTT
GGTCAACTATATTCCCCAGGGTTCAATAGATTCACTCTTTGAAGGAACGTGGTACTTAGTTAGGGTGGATGAAAAGCACAGAA
GAACTTACGCTCGGCGTCCCACTCCAAATGATGACACTTTGGATGAAGGAGTAGGACTTGTGCATTCAAACATAGCAACTGAG
CATATTCCAAGCCCTGCCAAGAAAGTACCAAGACTCCCTGCCACAGCAGCAGAACCTGAAGCAGCTGTCATTAGTAATGGGGA
ACATTAAGATACTCTGTGAGGTGCAAGACTTCAGCGTGGCGTGGGCATGGGGTGGGGGTATGGGAACAGTTCG

TABLE P3
Human Cytosolic HMG CoA synthase Protein Sequence

ORF Start: 22 ORF Stop: 1582 Frame: 1
CG97025-01-prot 520 aa

(SEQ ID NO:467)

MPGSLPLNAEACWPKDVGIVALEIYFPSQYVDQAELEKYDOVDAGKYTIGLCQAKMGFCTDREDINSLCMTVVQNLMERNN
LSYDCIGRLEVGTETITDKSKSVKTNLMQLFEESGNTDIEGIDTTNACYGQTAAVFNAVNWIESSSWDGRYALVVAGDIAV
YATGNARPTGGVGAVALLIGPNAPLIFERGLRGTHMQHAYDFYKPDMLSEYPIVDGKLSIQCYLSALDRCYSVYCKKIHAQ
WQKEGNDKDFTLNDFGFMIFHSPYCKLVQKSLARMLLNDFLNDQNRDKNSIYSGLEAFGDVKLEDTYFDRDVEKAFMKASS
ELFSQKTKASLLVSNQNGNMYTSSVYGSLASVLAQYSPQQLACKRIGVFSYGSGLAATLYSLKVTQDATPGSALDKITASL
CDLKSRLDSRTGVAPDVFAENMKLREDTHHLVNYIPQGSIDSLFEGTWYLVRVDEKHRRTYARRPTPNDDTLDEGVGLVHS
NIATEHIPSPAKKVPRLPATAAEPEAAVISNGEH

The following is an alignment of the protein sequences of the human (CG97025-01; SEQ ID NO:468), rat (J05210) and mouse (AF332052; SEQ ID NO:469) versions of the Cytosolic HMG CoA synthase.

[image]

The variants of the human Cytosolic HMG CoA synthase obtained from direct cloning and/or public databases. In addition to the human version of the Cytosolic HMG CoA synthase identified as being differentially expressed in the experimental study, no other variants have been identified by direct sequencing of cDNAs derived from many different human tissues and from sequences in public databases. No splice-form variants have been identified at CuraGen.

RTQ-PCR Results—Human Cytosolic HMG CoA synthase: The quantitative expression of various clones was assessed as described in Example C. Expression of gene CG97025-01 was assessed using the primer-probe set Ag4087, described in Table PAA. Results of the RTQ-PCR runs are shown in Tables PAB and PAC.

TABLE PAA

Probe Name Ag4087

| | | | |
|---|---|---|---|
| | | Start | SEQ ID |
| Primers | Length | Position | NO |

| | | | | |
|---|---|---|---|---|
| Forward | 5′-ttcagtatatggttcccttgca-3′ | 22 | 1062 | 470 |
| Probe | TET-5′-tgttctagcacagtactcacctcagca-3′-TAMRA | 27 | 1086 | 471 |
| Reverse | 5′-actccaattctcttccctgcta-3′ | 22 | 1115 | 472 |

TABLE PAB
General_screening_panel_v1.4

| | Reel. Exp. (%) | | Reel. Exp. (%) |
|---|---|---|---|
| | Ag4087, | | Ag4087, |
| | Run | | Run |
| Tissue Name | 219430028 | Tissue Name | 219430028 |
| Adipose | 2.3 | Renal ca. TK-10 | 24.7 |
| Melanoma* Hs688(A).T | 3.2 | Bladder | 17.6 |
| Melanoma* Hs688(B).T | 8.8 | Gastric ca. (liver met.) NCI-N87 | 23.3 |
| Melanoma* M14 | 18.6 | Gastric ca. KATO III | 79.6 |
| Melanoma* LOXIMVI | 4.4 | Colon ca. SW-948 | 14.2 |
| Melanoma* SK-MEL-5 | 21.6 | Colon ca. SW480 | 10.7 |
| Squamous cell carcinoma | 39.5 | Colon ca.* (SW480 met) SW620 | 9.5 |
| SCC-4 | | | |
| Testis Pool | 6.2 | Colon ca. HT29 | 20.4 |
| Prostate ca.* (bone met) PC-3 | 6.8 | Colon ca. HCT-116 | 24.8 |
| Prostate Pool | 0.6 | Colon ca. CaCo-2 | 63.3 |
| Placenta | 1.3 | Colon cancer tissue | 5.0 |
| Uterus Pool | 2.0 | Colon ca. SW1116 | 3.3 |
| Ovarian ca. OVCAR-3 | 80.7 | Colon ca. Colo-205 | 10.2 |
| Ovarian ca. SK-OV-3 | 26.6 | Colon ca. SW-48 | 7.9 |
| Ovarian ca. OVCAR-4 | 7.1 | Colon Pool | 2.8 |
| Ovarian ca. OVCAR-5 | 31.4 | Small Intestine Pool | 3.2 |
| Ovarian ca. IGROV-1 | 58.6 | Stomach Pool | 2.7 |
| Ovarian ca. OVCAR-8 | 3.5 | Bone Marrow Pool | 1.2 |
| Ovary | 11.4 | Fetal Heart | 4.1 |
| Breast ca. MCF-7 | 17.9 | Heart Pool | 1.5 |
| Breast ca. MDA-MB-231 | 12.9 | Lymph Node Pool | 2.9 |
| Breast ca. BT 549 | 38.7 | Fetal Skeletal Muscle | 0.2 |
| Breast ca. T47D | 55.9 | Skeletal Muscle Pool | 2.4 |
| Breast ca. MDA-N | 7.9 | Spleen Pool | 4.4 |
| Breast Pool | 2.4 | Thymus Pool | 3.3 |
| Trachea | 3.8 | CNS cancer (glio/astro) U87-MG | 10.4 |
| Lung | 1.2 | CNS cancer (glio/astro) | 8.7 |
| | | U-118-MG | |
| Fetal Lung | 9.9 | CNS cancer (neuro; met) | 19.3 |
| | | SK-N-AS | |
| Lung ca. NCI-N417 | 22.4 | CNS cancer (astro) SF-539 | 42.9 |
| Lung ca. LX-1 | 16.8 | CNS cancer (astro) SNB-75 | 26.1 |
| Lung ca. NCI-H146 | 28.5 | CNS cancer (glio) SNB-19 | 51.8 |
| Lung ca. SHP-77 | 36.6 | CNS cancer (glio) SF-295 | 11.4 |
| Lung ca. A549 | 25.2 | Brain (Amygdala) Pool | 11.3 |
| Lung ca. NCI-H526 | 25.7 | Brain (cerebellum) | 3.3 |
| Lung ca. NCI-H23 | 16.7 | Brain (fetal) | 52.5 |
| Lung ca. NCI-H460 | 4.5 | Brain (Hippocampus) Pool | 17.7 |
| Lung ca. HOP-62 | 23.0 | Cerebral Cortex Pool | 17.8 |
| Lung ca. NCI-H522 | 9.2 | Brain (Substantia nigra) Pool | 15.9 |
| Liver | 1.3 | Brain (Thalamus) Pool | 26.2 |
| Fetal Liver | 100.0 | Brain (whole) | 14.9 |
| Liver ca. HepG2 | 50.7 | Spinal Cord Pool | 13.2 |
| Kidney Pool | 6.0 | Adrenal Gland | 23.0 |
| Fetal Kidney | 8.8 | Pituitary gland Pool | 1.2 |
| Renal ca. 786-0 | 31.0 | Salivary Gland | 0.8 |
| Renal ca. A498 | 4.1 | Thyroid (female) | 2.1 |
| Renal ca. ACHN | 20.9 | Pancreatic ca. CAPAN2 | 56.6 |
| Renal ca. UO-31 | 18.6 | Pancreas Pool | 4.9 |

TABLE PAC
Panel 5 Islet

| | Rel. | | Rel. |
|---|---|---|---|
| | Exp. (%) | | Exp. (%) |
| | Ag4087, | | Ag4087, |
| | Run | | Run |
| Tissue Name | 186511156 | Tissue Name | 186511156 |
| 97457_Patient-02go_adipose | 1.8 | 94709_Donor 2 AM - A_adipose | 10.6 |
| 97476_Patient-07sk_skeletal | 2.3 | 94710_Donor 2 AM - B_adipose | 7.2 |
| muscle | | | |
| 97477_Patient-07ut_uterus | 3.6 | 94711_Donor 2 AM - C_adipose | 2.6 |
| 97478_Patient-07pl_placenta | 5.5 | 94712_Donor 2 AD - A_adipose | 14.0 |
| 99167_Bayer Patient 1 | 13.8 | 94713_Donor 2 AD - B_adipose | 13.7 |
| 97482_Patient-08ut_uterus | 1.3 | 94714_Donor 2 AD - C_adipose | 14.8 |
| 97483_Patient-08pl_placenta | 4.5 | 94742_Donor 3 U - A_Mesenchymal | 7.2 |
| | | Stem Cells | |
| 97486_Patient-09sk_skeletal | 0.4 | 94743_Donor 3 U - B_Mesenchymal | 8.5 |
| muscle | | Stem Cells | |
| 97487_Patient-09ut_uterus | 3.0 | 94730_Donor 3 AM - A_adipose | 12.9 |
| 97488_Patient-09pl_placenta | 3.5 | 94731_Donor 3 AM - B_adipose | 7.9 |
| 97492_Patient-10ut_uterus | 2.7 | 94732_Donor 3 AM - C_adipose | 7.7 |
| 97493_Patient-10pl_placenta | 12.6 | 94733_Donor 3 AD - A_adipose | 28.9 |
| 97495_Patient-11go_adipose | 2.2 | 94734_Donor 3 AD - B_adipose | 5.6 |
| 97496_Patient-11sk_skeletal | 2.9 | 94735_Donor 3 AD - C_adipose | 23.8 |
| muscle | | | |
| 97497_Patient-11ut_uterus | 4.5 | 77138_Liver_HepG2untreated | 100.0 |
| 97498_Patient-11pl_placenta | 3.3 | 73556_Heart_Cardiac stromal cells | 2.9 |
| | | (primary) | |
| 97500_Patient-12go_adipose | 5.2 | 81735_Small Intestine | 10.3 |
| 97501_Patient-12sk_skeletal | 6.2 | 72409_Kidney_Proximal Convoluted | 8.8 |
| muscle | | Tubule | |
| 97502_Patient-12ut_uterus | 4.7 | 82685_Small intestine_Duodenum | 1.8 |
| 97503_Patient-12pl_placenta | 6.2 | 90650_Adrenal_Adrenocortical | 10.2 |
| | | adenoma | |
| 94721_Donor 2 U - | 7.9 | 72410_Kidney_HRCE | 42.6 |
| A_Mesenchymal Stem Cells | | | |
| 94722_Donor 2 U - | 5.0 | 72411_Kidney_HRE | 38.2 |
| B_Mesenchymal Stem Cells | | | |
| 94723_Donor 2 U - | 9.5 | 73139_Uterus_Uterine smooth | 4.7 |
| C_Mesenchymal Stem Cells | | muscle cells | |

General_screening_panel_v1.4 Summary: Method of Use Panel

Panel 5 Islet Summary: Method of Use Panel

Biochemistry and Cell Line Expression

The following summarizes the biochemistry surrounding the human Cytosolic HMG CoA synthase enzyme. Cell lines expressing the Cytosolic HMG CoA synthase enzyme can be obtained from the RTQ-PCR results shown above. These and other Cytosolic HMG CoA synthase enzyme expressing cell lines could be used for screening purposes.

Biochemistry: Cytosolic HMG CoA synthase condenses acetyl-CoA with acetoacetyl-CoA to form HMG-CoA, which is the substrate for HMG-CoA Reductase. This condensation reaction occurs above the diversion point to farnesoic acid in the cholesterol biosynthetic pathway. The reaction proceeds as follows:

acetyl-CoA+H₂O+acetoacetyl-CoA=(S)-3-hydroxy-3-methylglutaryl-CoA+CoA

Rationale for Use as a Diagnostic and/or Target for Small Molecule Drugs and Antibody Therapeutics.

HMG CoA synthase is up-regulated 7-fold in a genetic model of obesity characterized by apparent LXR alpha activation (adipose induction of ApoE, malic enzyme, ATP citrate lyase, FAS, SCD), thus HMG CoA synthase provides the substrate for LXRa ligands. Inhibition of this enzyme may be a treatment for the prevention or treatment of obesity.

Taken in total, the data indicates that an inhibitor of the human Cytosolic HMG CoA synthase enzyme would be beneficial in the treatment of obesity and/or diabetes.

[image]

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO:2n, wherein n is an integer between 1 and 188.

2. An isolated polypeptide comprising an amino acid sequence selected from the group consisting of SEQ ID NO:2n, wherein n is an integer between 1 and 188.

3. An isolated polypeptide comprising an amino acid sequence which is at least 95% identical to an amino acid sequence selected from the group consisting of SEQ ID NO:2n, wherein n is an integer between 1 and 188.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 188.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 188 or a biologically active fragment thereof.

20. An isolated nucleic acid molecule comprising a nucleic acid sequence selected from the group consisting of SEQ ID NO:2n−1, wherein n is an integer between 1 and 188.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 188.

23. An isolated nucleic acid molecule encoding the mature form of a polypeptide having an amino acid sequence selected from the group consisting of SEQ ID NO:2n, wherein n is an integer between 1 and 188.

24. An isolated nucleic acid molecule comprising a nucleic acid selected from the group consisting of 2n−1, wherein n is an integer between 1 and 188.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 188, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 188.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 188.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
